(19) United States
(12) Patent Application Publication
(10) Pub. No.: US 2025/0170235 A1
(43) Pub. Date: May 29, 2025

(54) MESSENGER RIBONUCLEIC ACIDS FOR ENHANCING IMMUNE RESPONSES AND METHODS OF USE THEREOF

(71) Applicant: ModernaTX, Inc., Cambridge, MA (US)

(72) Inventors: Eric Yi-Chun HUANG, Cambridge, MA (US); Sze-Wah TSE, Cambridge, MA (US); Jared IACOVELLI, Cambridge, MA (US); Kristine MCKINNEY, Cambridge, MA (US); Kristen HOPSON, Cambridge, MA (US)

(21) Appl. No.: 18/756,934

(22) Filed: Jun. 27, 2024

Related U.S. Application Data
(63) Continuation of application No. 18/517,361, filed on Nov. 22, 2023, now abandoned.
Application No. 18/517,361 is a continuation of application No. 16/671,921, filed on Nov. 1, 2019, now abandoned.
Application No. 16/671,921 is a continuation of application No. 15/995,519, filed on Jun. 1, 2018, now abandoned.
Application No. 15/995,519 is a continuation of application No. PCT/US2017/058585, filed on Oct. 26, 2017.
(60) Provisional application No. 62/558,206, filed on Sep. 13, 2017.
(60) Provisional application No. 62/467,034, filed on Mar. 3, 2017.
(60) Provisional application No. 62/490,522, filed on Apr. 26, 2017.
(60) Provisional application No. 62/412,933, filed on Oct. 26, 2016.

(51) Int. Cl. A61K 39/39; A61K 9/51; A61K 31/7088; A61K 39/00; A61K 39/02; A61K 39/12; A61K 47/14; A61P 35/00; C07K 14/47; C12N 7/00

(52) U.S. Cl. CPC A61K 39/39; A61K 9/5123; A61K 31/7088; A61K 39/0011; A61K 39/001164; A61K 39/02; A61K 39/12; A61K 47/14; A61P 35/00; C07K 14/4705; C12N 7/00; A61K 2039/53; A61K 2039/545; A61K 2039/572; A61K 2039/575; A61K 2039/70; C12N 2710/20034; C12N 2710/20071

(57) ABSTRACT

The disclosure features isolated mRNAs encoding a polypeptide that enhances immune responses to an antigen(s) of interest, such as polypeptides that activate Type I interferon pathway signaling or NFkB signaling, including mRNAs comprising one or more modified nucleobase. The disclosure also features methods of using the same, for example, for enhancing immune responses when administered with an antigen(s) of interest, such as to stimulate anti-cancer immune responses or anti-pathogen immune responses.

### RELATED APPLICATIONS

This application is a continuation of U.S. application Ser. No. 18/517,361, filed Nov. 22, 2023, which is a continuation of U.S. application Ser. No. 16/671,921, filed Nov. 1, 2019, which is a continuation of U.S. application Ser. No. 15/995,519, filed Jun. 1, 2018, which is a continuation of International Application No. PCT/US2017/058585, filed Oct. 26, 2017, which claims the benefit of U.S. Provisional Application No. 62/412,933 filed on Oct. 26, 2016; U.S. Provisional Application No. 62/467,034 filed on Mar. 3, 2017; U.S. Provisional Application No. 62/490,522 filed on Apr. 26, 2017; and U.S. Provisional Application No. 62/558,206 filed on Sep. 13, 2017. The entire contents of the above-referenced applications are incorporated herein by this reference.

### STATEMENT REGARDING SEQUENCE LISTING

The Sequence Listing XML associated with this application is provided electronically in XML file format and is hereby incorporated by reference into the specification. The name of the XML file containing the Sequence Listing XML is “MRNA-105_C03US_SeqList.xml”. The XML file is 2,146,937 bytes, created on Nov. 21, 2023, and is being submitted electronically via USPTO Patent Center.

### BACKGROUND OF THE DISCLOSURE

The ability to modulate an immune response is beneficial in a variety of clinical situations, including the treatment of cancer and pathogenic infections, as well as in potentiating vaccine responses to provide protective immunity. A number of therapeutic tools exist for modulating the function of biological pathways and/or molecules that are involved in diseases such as cancer and pathogenic infections. These tools include, for example, small molecule inhibitors, cytokines and therapeutic antibodies. Some of these tools function through modulating immune responses in a subject, such as cytokines that modulate the activity of cells within the immune system or immune checkpoint inhibitor antibodies, such as anti-CTLA-4 or anti-PD-L1 that modulate the regulation of immune responses.

Additionally, vaccines have long been used to stimulate an immune response against antigens of pathogens to thereby provide protective immunity against later exposure to the pathogens. More recently, vaccines have been developed using antigens found on tumor cells to thereby enhance anti-tumor immunoresponsiveness. In addition to the antigen(s) used in the vaccine, other agents may be included in a vaccine preparation, or used in combination with the vaccine preparation, to further boost the immune response to the vaccine. Such agents that enhance vaccine responsiveness are referred to in the art as adjuvants. Examples of commonly used vaccine adjuvants include aluminum gels and salts, monophosphoryl lipid A, MF59 oil-in-water emulsion, Freund's complete adjuvant, Freund's incomplete adjuvant, detergents and plant saponins. These adjuvants typically are used with protein or peptide based vaccines. Alternative types of vaccines, such as RNA based vaccines, are now being developed.

There exists a need in the art for additional effective agents that enhance immune responses to an antigen of interest.

### SUMMARY OF THE DISCLOSURE

This disclosure provides messenger RNAs (mRNAs) encoding a polypeptide that enhances an immune response to an antigen(s) of interest, referred to herein as immune potentiator constructs. In certain embodiments, the messenger RNAs (mRNAs) are chemically modified, referred to herein as a modified mRNA (mmRNA), wherein the mmRNA comprises one or more modified nucleobases. Alternatively, the mRNA can entirely comprise unmodified nucleobases. In one embodiment, an immune potentiator construct pertains to a messenger RNA (mRNA) encoding a polypeptide that enhances an immune response to an antigen of interest in a subject (optionally wherein said mRNA comprises one or more modified nucleobases), and wherein the immune response comprises a cellular or humoral immune response characterized by:
(i) stimulating Type I interferon pathway signaling;
(ii) stimulating NFkB pathway signaling;
(iii) stimulating an inflammatory response;
(iv) stimulating cytokine production; or
(v) stimulating dendritic cell development, activity or mobilization; and
(vi) a combination of any of (i)-(vi).

In certain embodiments, the immune potentiator mRNA construct (or combination of immune potentiator mRNA constructs) enhances an immune response to an antigen of interest by a fold magnitude, e.g., relative to the immune response to the antigen in the absence of the immune potentiator, or relative to a small molecular agonist that enhances an immune response to the antigen. For example, in various embodiments, the immune potentiator mRNA construct enhances an immune response to an antigen of interest by 0.3-1000 fold, 1-750 fold, 5-500 fold, 7-250 fold, or 10-100 fold as compared to, for example, the immune response to the antigen in the absence of the immune potentiator mRNA construct or as compared to, for example, the immune response to the antigen in the presence of a small molecular agonist of an immune response to the antigen. In some embodiments, the immune potentiator mRNA construct enhances an immune response to an antigen of interest by at least 2-fold, 3-fold, 4-fold, 5-fold, 7.5-fold, 10-fold, 20-fold, 30-fold, 40-fold, 50-fold, 75-fold, or greater, as compared to, for example, the immune response to the antigen in the absence of the immune potentiator mRNA construct or as compared to, for example, the immune response to the antigen in the presence of a small molecular agonist of an immune response to the antigen.

The antigen of interest can be an endogenous antigen in a subject (e.g., an endogenous tumor antigen) or an exogenous antigen that is provided to the subject with the immune potentiator construct (e.g., an exogenous tumor antigen or pathogen antigen, including vaccine antigens). Thus, the immune potentiator mRNAs of the disclosure are useful to stimulate or potentiate an immune response in vivo against antigens of interest, such as tumor antigens in the treatment of cancer or pathogen antigens in the treatment of or vaccination against pathogenic diseases.

In one embodiment, the antigen of interest is an endogenous antigen, such as a tumor antigen and the mRNA immune potentiator construct is provided to a subject in need thereof to stimulate or potentiate an immune response against the tumor antigen. In certain embodiments, the mRNA immune potentiator construct is administered in combination with one or more additional agents, e.g., mRNA constructs, to promote the release of endogenous antigens, for example by inducing immunogenic cell death, such as by necroptosis or pyroptosis. Accordingly, in another aspect, the invention provides mRNA constructs (e.g., mmRNAs) that encode a polypeptide that induces immunogenic cell death, such as necroptosis or pyroptosis. In some aspects, the immunogenic cell death induced by the mRNAs results in release of cytosolic components from the cell (e.g., a tumor cell) such that an immune response against cellular antigens (e.g., endogenous tumor antigens) is stimulated in vivo.

In other embodiments, the antigen of interest is an exogenous antigen that is encoded by an mRNA, such as a chemically modified mRNA (mmRNA), provided on the same mRNA as the immune potentiator construct or provided on a different mRNA construct as the immune potentiator. The immune potentiator and antigen mRNAs are formulated (or coformulated) and administered (simultaneously or sequentially) to a subject in need thereof to stimulate an immune response against the antigen in the subject.

In some aspects, the disclosure provides an immune potentiator mRNA (e.g., mmRNA construct) which encodes a polypeptide that enhances an immune response by, for example, stimulating Type I interferon pathway signaling, stimulating NFkB pathway signaling, stimulating an inflammatory response, stimulating cytokine production or stimulating dendritic cell development, activity or mobilization. Enhancement of an immune response to an antigen of interest by an immune potentiator mRNA results in, for example, stimulation of cytokine production, stimulation of cellular immunity (T cell responses), such as antigen-specific CD8⁺ or CD4⁺ T cell responses and/or stimulation of humoral immunity (B cell responses), such as antigen-specific antibody responses, or any combination of the foregoing responses.

In some aspects, the disclosure provides an immune potentiator mRNA (e.g., mmRNA) encoding a polypeptide that functions downstream of at least one Toll-like receptor (TLR) to thereby enhance an immune response, examples of which are provided herein. In some aspects, the disclosure provides an immune potentiator mRNA (e.g., mmRNA) encoding a polypeptide that stimulates a Type I interferon response, examples of which are provided herein. In some aspects, the disclosure provides an immune potentiator mRNA (e.g., mmRNA) encoding a polypeptide that stimulates an NFkB-mediated proinflammatory response, examples of which are provided herein. In some aspects, the disclosure provides an immune potentiator mRNA (e.g., mmRNA) encoding a polypeptide that is an intracellular adaptor protein, examples of which are provided herein. In some aspects, the disclosure provides an immune potentiator mRNA (e.g., mmRNA) encoding a polypeptide that is an intracellular signaling protein, examples of which are provided herein. In some aspects, the disclosure provides an immune potentiator mRNA (e.g., mmRNA) encoding a polypeptide that is a transcription factor, examples of which are provided herein. In some aspects, the disclosure provides an immune potentiator mRNA (e.g., mmRNA) encoding a polypeptide that is involved in necroptosis or necroptosome formation, examples of which are provided herein. In some aspects, the disclosure provides an immune potentiator mRNA (e.g., mmRNA) encoding a polypeptide that is involved in pyroptosis or inflammasome formation, examples of which are provided herein. Compositions that comprise combinations of two or more immune potentiator mRNAs (of the same class type or of different class types) are also provided.

In some aspects, the disclosure provides an immune potentiator mRNA (e.g., mmRNA) encoding a constitutively active human STING polypeptide. In one aspect, the constitutively active human STING polypeptide comprises one or more mutations selected from the group consisting of V147L, N154S, V155M, R284M, R284K, R284T, E315Q, R375A, and combinations thereof. In some aspects, the constitutively active human STING polypeptide comprises a V155M mutation (e.g., having the amino acid sequence shown in SEQ ID NO: 1 or encoded by a nucleotide sequence shown in SEQ ID NO: 199, 1319 or 1320). In some aspects, the constitutively active human STING polypeptide comprises mutations V147L/N154S/V155M. In other aspects, the constitutively active human STING polypeptide comprises mutations R284M/V147L/N154S/V155M. In other aspects, the constitutively active human STING polypeptide comprises an amino acid sequence set forth in any one of SEQ ID NOs: 1-10 and 224. In another aspect, the constitutively active human STING polypeptide is encoded by a nucleotide sequence set forth in any one of SEQ ID NOs: 199-208, 225, 1319, 1320, 1442-1450 and 1466.

In other aspects, the disclosure provides an immune potentiator mRNA (e.g., mmRNA) encoding a constitutively active human IRF3 polypeptide. In one aspect, the constitutively active human IRF3 polypeptide comprises an S396D mutation. In one aspect, the constitutively active human IRF3 polypeptide comprises an amino acid sequence set forth in SEQ ID NO: 11 or is encoded by a nucleotide sequence set forth in SEQ ID NO: 210 or SEQ ID NO: 1452. In one aspect, the constitutively active IRF3 polypeptide is a mouse IRF3 polypeptide, for example comprising an amino acid sequence set forth in SEQ ID NO: 12 or encoded by the nucleotide sequence shown in SEQ ID NO: 211 or SEQ ID NO: 1453.

In yet other aspects, the disclosure provides an immune potentiator mRNA (e.g., mmRNA) encoding a constitutively active human IRF7 polypeptide. In one aspect, the constitutively active human IRF7 polypeptide comprises one or more mutations selected from the group consisting of S475D, S476D, S477D, S479D, L480D, S483D, S487D, and combinations thereof; deletion of amino acids 247-467; and combinations of the foregoing mutations and/or deletions. In one embodiment, the constitutively active human IRF7 polypeptide comprises an amino acid sequence set forth in any one of SEQ ID NOs: 14-18. In one embodiment, the constitutively active human IRF7 polypeptide is encoded by a nucleotide sequence set forth in any one of SEQ ID NOs: 213-217 and 1454-1459.

In yet other aspects, the disclosure provides an immune potentiator mRNA (e.g., mmRNA) encoding a polypeptide selected from the group consisting of MyD88, TRAM, IRF1, IRF8, IRF9, TBK1, IKKi, STAT1, STAT2, STAT4, STAT6, c-FLIP, IKKα, IKKβ, RIPK1, TAK-TAB1 fusion, DIABLO, Btk, self-activating caspase-1 and Flt3. In other aspects, the disclosure provides mRNA compositions (e.g., mmRNA compositions) comprising one or more mRNA constructs (e.g., mmRNA constructs), encoding an antigen(s) of interest and a polypeptide that enhances an immune response against the antigen(s) of interest, wherein the antigen(s) and the polypeptide are encoded either by the same mRNA (mmRNA) construct or separate mRNA (mmRNA) constructs that can be coformulated and administered, simultaneously or sequentially to a subject in need thereof. Any of the immune potentiator mRNAs (e.g., mmRNAs) described herein (alone or in combination) are useful in one or more compositions for enhancing an immune response to an antigen(s) of interest.

Accordingly, in some aspects, the disclosure provides a composition comprising a first mRNA (e.g., mmRNA) encoding a polypeptide that enhances an immune response and a second mRNA (e.g., mmRNA) encoding at least one antigen of interest, optionally wherein said first and second mRNAs comprise one or more modified nucleobases, and wherein the polypeptide enhances an immune response to the at least one antigen of interest when the composition is administered to a subject. In one aspect, the composition comprises a single mRNA construct (e.g., mmRNA) encoding both the at least one antigen of interest and the polypeptide that enhances an immune response to the at least one antigen of interest. In another aspect, the composition comprises two mRNA constructs (e.g., mmRNAs), one encoding the at least one antigen of interest and the other encoding the polypeptide that enhances an immune response to the at least one antigen of interest. In some aspects, when the composition comprises two mRNA constructs, the two mRNA constructs (e.g., mmRNAs) are coformulated in the same composition (such as, for example, a lipid nanoparticle) and coadministered to a subject. In other aspects when two or more mRNA constructs are provided, such mRNA constructs can be formulated in different compositions (such as, for example, two or more lipid nanoparticles) and administered (e.g., simultaneously or sequentially) to a subject in need thereof.

In other aspects, the disclosure provides a composition comprising a first mRNA (e.g., mmRNA) encoding a polypeptide that enhances an immune response and a second mRNA (e.g., mmRNA) encoding at least one antigen of interest, wherein the at least one antigen of interest is at least one tumor antigen. In one aspect, the at least one tumor antigen is at least one mutant KRAS antigen. In one aspect, the at least one mutant KRAS antigen comprises at least one mutation selected from the group consisting of G12D, G12V, G13D, G12C and combinations thereof. In one aspect, the at least one mutant human KRAS antigen comprises an amino acid sequence as set forth in any one of SEQ ID NOs: 95-106 and 131-132. In other aspects, the composition comprises an mRNA construct encoding at least one mutant human KRAS antigen and a constitutively active human STING polypeptide, for example wherein the mRNA encodes an amino acid sequence as set forth in any one of SEQ ID NOs: 107-130. Exemplary mRNA nucleotide sequences for constructs encoding at least one mutant human KRAS antigen and a constitutively active human STING polypeptide are shown in SEQ ID NOs: 220-223 and 1462-1465. In other aspects, the tumor antigen is an oncovirus antigen (e.g., a human papilloma virus (HPV) antigen, such as HPV16 E6 or HPV E7 antigen, or combination thereof).

In other aspects of the composition of the disclosure, the at least one antigen of interest is at least one pathogen antigen. In one aspect, the at least one pathogen antigen is from a pathogen selected from the group consisting of viruses, bacteria, protozoa, fungi and parasites. In one embodiment, the at least one pathogen antigen is at least one viral antigen. In one aspect, the at least one viral antigen is at least one human papillomavirus (HPV) antigen. In one aspect, the HPV antigen is an HPV16 E6 or HPV E7 antigen, or combination thereof. In one aspect, the HPV antigen comprises an amino acid sequence as set forth in in any one of SEQ ID NOs: 36-94. In other aspects of the composition of the disclosure, the at least one pathogen antigen is at least one bacterial antigen. In one embodiment, the at least one bacterial antigen is a multivalent antigen.

In one embodiment, the antigen of interest is one or more antigens of an oncogenic virus, such as human papilloma virus (HPV), Hepatitis B Virus (HBV), Hepatitis C Virus (HCV), Epstein Barr Virus (EBV), Human T-cell Lymphotropic Virus Type I (HTLV-I), Kaposi's sarcoma herpesvirus (KSHV) or Merkel cell polyomavirus (MCV). In one aspect, an antigen of interest of an oncogenic virus is encoded by an mRNA (e.g., a chemically modified mRNA), and provided on the same mRNA as the immune potentiator construct or provided on a different mRNA construct as the immune potentiator. In some aspects, the immune potentiator and viral antigen(s) mRNAs are formulated (or coformulated) and administered (concurrently or sequentially) to a subject in need thereof to stimulate an immune response against the oncogenic viral antigen(s) in the subject. Suitable oncogenic viral antigens for use with the immune potentiators are described herein.

In one embodiment, the antigen of interest is one or more tumor antigens that comprise a personalized cancer vaccine. In one aspect, the disclosure provides a vaccine preparation that includes mRNA (e.g., mmRNA) encoding for one or more cancer antigens specific for the cancer subject, referred to as neoepitopes, along with an immune potentiator construct, wherein the cancer antigens and the immune potentiator are encoded by the same or different mRNAs (e.g., mmRNAs). Methods of selecting cancer antigens specific for a cancer subject, and designing personalized cancer vaccines based thereon, are described herein. Accordingly, in one aspect, the disclosure provides a personalized cancer vaccine comprising one or more tumor antigens specific for a cancer subject (e.g., one or more neoepitopes), encoded by one or more mRNAs (e.g., chemically modified mRNAs), wherein the cancer neoepitopes are encoded by the same mRNA or different mRNAs (e.g., each cancer neoepitope is encoded on a separate mRNA construct). In some aspects, the cancer neoepitope(s) are encoded on the same mRNA construct as the immune potentiator construct or encoded on a different mRNA construct as the immune potentiator. The immune potentiator and cancer antigen(s) mRNAs can be formulated (or coformulated) and administered (concurrently or sequentially) to a subject in need thereof to stimulate an immune response against the cancer antigen(s) in the subject.

In one aspect, the mRNA construct encodes a personalized cancer antigen which is a concatemeric cancer antigen comprised of 2-100 peptide epitopes. In another aspect, the concatemeric cancer antigen comprises one or more of: a) the 2-100 peptide epitopes are interspersed by cleavage sensitive sites; b) the mRNA encoding each peptide epitope is linked directly to one another without a linker; c) the mRNA encoding each peptide epitope is linked to one or another with a single nucleotide linker; d) each peptide epitope comprises 25-35 amino acids and includes a centrally located SNP mutation; e) at least 30% of the peptide epitopes have a highest affinity for class I MHC molecules from a subject; f) at least 30% of the peptide epitopes have a highest affinity for class II MHC molecules from a subject; g) at least 50% of the peptide epitopes have a predicated binding affinity of IC >500 nM for HLA-A, HLA-B and/or DRB1; h) the mRNA encodes 20 peptide epitopes; i) 50% of the peptide epitopes have a binding affinity for class I MHC and 50% of the peptide epitopes have a binding affinity for class II MHC; and/or j) the mRNA encoding the peptide epitopes is arranged such that the peptide epitopes are ordered to minimize pseudo-epitopes.

In some aspects, the concatemeric cancer antigen comprises 2-100 peptide epitopes, wherein each peptide epitope comprises 31 amino acids and includes a centrally located SNP mutation with 15 flanking amino acids on each side of the SNP mutation. In some aspects, the peptide epitopes are T cell epitopes, B cell epitopes or a combination of T cell epitopes and B cell epitopes. In some aspects, the peptide epitopes comprise at least one MHC class I epitope and at least one MHC class II epitope. In some aspects, at least 30% of the epitopes are MHC class I epitopes or at least 30% of the epitopes are MHC class II epitopes.

In one embodiment, the antigen of interest is at least one bacterial antigen, for example a bacterial vaccine that comprises at least one bacterial antigen and an immune potentiator construct, encoded on the same or separate mRNAs (e.g., mmRNAs). In one aspect, the disclosure provides a bacterial vaccine that includes mRNA encoding for one or more bacterial antigens along with an immune potentiator construct, wherein the bacterial antigens and the immune potentiator are encoded by the same or different mRNAs. Accordingly, in one aspect, the disclosure provides a bacterial vaccine comprising one or more bacterial antigens (e.g., a multivalent vaccine), (e.g., encoded by one or more chemically modified mRNAs), wherein the bacterial antigens are encoded by the same mRNA or different mRNAs (e.g., each bacterial antigen is encoded on a separate mRNA construct). In some aspects, the bacterial antigens are encoded on the same mRNA construct as the immune potentiator construct or encoded on a different mRNA construct as the immune potentiator. The immune potentiator and bacterial antigen(s) mRNAs can be formulated (or coformulated) and administered (concurrently or sequentially) to a subject in need thereof to stimulate an immune response against the bacterial antigen(s) in the subject

In some embodiments, the bacterial vaccine is administered to a subject to provide prophylactic treatment (i.e., prevents infection). In some embodiments, the bacterial vaccine is administered to a subject to provide therapeutic treatment (i.e., treats infection). In some embodiments, the bacterial vaccine induces a humoral immune response in the subject (i.e., production of antibodies specific for the bacterial antigen of interest). In some embodiments, the bacterial vaccine induces an adaptive immune response in the subject. Non-limiting examples of suitable bacteria include Staphylococcus aureus.

In one embodiment, the antigen of interest is a multivalent antigen, (i.e., the antigen comprises multiple antigenic epitopes, such as multiple antigenic peptides comprising the same or different epitopes) to thereby enhance an immune response against the multivalent antigen. In one aspect, the multivalent antigen is a concatemeric antigen. In some embodiments, the mRNA vaccines described herein comprise an mRNA having an open reading frame encoding a concatemeric antigen comprised of 2-100 peptide epitopes (e.g., the same or different epitopes). In one embodiment, the multivalent antigen is a cancer antigen. In another embodiment, the multivalent antigen is a bacterial antigen. Non-limiting examples of multivalent antigens are described herein.

An mRNA (e.g., mmRNA) construct of the disclosure (e.g., an immune potentiator mRNA, antigen-encoding mRNA, or combination thereof) can comprise, for example, a 5′ UTR, a codon optimized open reading frame encoding the polypeptide, a 3′ UTR and a 3′ tailing region of linked nucleosides. In one embodiment, the mRNA further comprises one or more microRNA (miRNA) binding sites.

In one embodiment, a modified mRNA construct of the disclosure is fully modified. For example, in one embodiment, the mmRNA comprises pseudouridine (w), pseudouridine (ψ) and 5-methyl-cytidine (m⁵C), 1-methyl-pseudouridine (m¹ψ), 1-methyl-pseudouridine (m¹ψ) and 5-methyl-cytidine (m⁵C), 2-thiouridine (s²U), 2-thiouridine and 5-methyl-cytidine (m⁵C), 5-methoxy-uridine (mo⁵U), 5-methoxy-uridine (mo⁵U) and 5-methyl-cytidine (m⁵C), 2′-O-methyl uridine, 2′-O-methyl uridine and 5-methyl-cytidine (m⁵C), N6-methyl-adenosine (m⁶A) or N6-methyl-adenosine (m⁶A) and 5-methyl-cytidine (m⁵C). In another embodiment, the mmRNA comprises pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine, or combinations thereof. In yet another embodiment, the mmRNA comprises 1-methyl-pseudouridine (m¹ψ), 5-methoxy-uridine (mo⁵U), 5-methyl-cytidine (m⁵C), pseudouridine (ψ), α-thio-guanosine, or α-thio-adenosine, or combinations thereof.

In another aspect, the disclosure pertains to a lipid nanoparticle comprising an mRNA (e.g., modified mRNA) of the disclosure. In one embodiment, the lipid nanoparticle is a liposome. In another embodiment, the lipid nanoparticle comprises a cationic and/or ionizable lipid. In one embodiment, the cationic and/or ionizable lipid is 2,2-dilinoleyl-4-methylaminoethyl-[1,3]-dioxolane (DLin-KC2-DMA) or dilinoleyl-methyl-4-dimethylaminobutyrate (DLin-MC3-DMA). In one embodiment, the lipid nanoparticle further comprises a targeting moiety conjugated to the outer surface of the lipid nanoparticle.

In another aspect, the disclosure pertains to a pharmaceutical composition comprising an mRNA (e.g., mmRNA) of the disclosure or a lipid nanoparticle of the disclosure, and a pharmaceutically acceptable carrier, diluent or excipient.

In some aspects, the disclosure provides an immunomodulatory therapeutic composition of any one of the foregoing or related embodiments, wherein each mRNA is formulated in the same or different lipid nanoparticle carrier. In some aspects, each mRNA encoding an antigen(s) of interest (e.g., cancer antigen, viral antigen, bacterial antigen) is formulated in the same or different lipid nanoparticle carrier. In some aspects, each mRNA encoding the immune potentiator that enhances an immune response to the antigen(s) of interest is formulated in the same or different lipid nanoparticle carrier. In some aspects, each mRNA encoding an antigen(s) of interest is formulated in the same lipid nanoparticle carrier and each mRNA encoding an immune potentiator is formulated in a different lipid nanoparticle carrier. In some aspects, each mRNA encoding the antigen(s) of interest is formulated in the same lipid nanoparticle carrier and each mRNA encoding an immune potentiator is formulated in the same lipid nanoparticle carrier as each mRNA encoding the antigen(s) of interest. In some aspects, each mRNA encoding an antigen(s) of interest is formulated in a different lipid nanoparticle carrier and each mRNA encoding immune potentiator is formulated in the same lipid nanoparticle carrier as each mRNA encoding each antigen(s) of interest (e.g., cancer antigen, viral antigen, bacterial antigen).

In some aspects, the disclosure provides an immunomodulatory therapeutic composition of any one of the foregoing embodiments, wherein the immunomodulatory therapeutic composition is formulated in a lipid nanoparticle, wherein the lipid nanoparticle comprises a molar ratio of about 20-60% ionizable amino lipid: 5-25% phospholipid: 25-55% sterol; and 0.5-15% PEG-modified lipid. In some aspects, the ionizable amino lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di ((Z)-non-2-en-1-yl) 9-((4-(dimethylamino) butanoyl)oxy) heptadecanedioate (L319).

In some aspects, the disclosure provides an immunomodulatory therapeutic composition of any one of the foregoing or related embodiments, wherein each mRNA includes at least one chemical modification. In some aspects, the chemical modification is selected from the group consisting of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine.

In other aspects, the disclosure provides a lipid nanoparticle carrier comprising a pharmaceutical composition, wherein the pharmaceutical composition comprises:
(i) an mRNA having an open reading frame encoding an HPV antigen; or
an mRNA having an open reading frame encoding an HPV16 antigen; or
an mRNA having an open reading frame encoding an HPV18 antigen; or
an mRNA having an open reading frame encoding at least one HPV E6 antigen; or
an mRNA having an open reading frame encoding at least one HPV E7 antigen; or
an mRNA having an open reading frame encoding at least one HPV E6 antigen and at least one HPV E7 antigen; and
(ii) an mRNA having an open reading frame encoding a constitutively active human STING polypeptide; and
a pharmaceutically acceptable carrier or excipient.

In some aspects of the foregoing lipid nanoparticle carrier, the constitutively active human STING polypeptide comprises mutation V155M. In some aspects, the constitutively active human STING polypeptide comprises the amino acid sequence shown in SEQ ID NO: 1. In some aspects, the mRNA encoding the constitutively active human STING polypeptide comprises a 3′ UTR comprising at least one miR-122 microRNA binding site. In some aspects, the mRNA encoding the constitutively active human STING polypeptide comprises the nucleotide sequence shown in SEQ ID NO: 199, 1319 or 1320.

In some aspects, the disclosure provides a lipid nanoparticle of any one of the foregoing embodiments, wherein the lipid nanoparticle comprises a molar ratio of about 20-60% ionizable amino lipid: 5-25% phospholipid: 25-55% sterol; and 0.5-15% PEG-modified lipid. In some aspects, the ionizable amino lipid is selected from the group consisting of for example, 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di ((Z)-non-2-en-1-yl) 9-((4-(dimethylamino) butanoyl)oxy) heptadecanedioate (L319). In certain embodiments, the lipid nanoparticle comprises Compound 25 (as the ionizable amino lipid), DSPC (as the phospholipid), cholesterol (as the sterol) and PEG-DMG (as the PEG-modified lipid). In certain embodiments, the lipid nanoparticle comprises a molar ratio of about 20-60% Compound 25:5-25% DSPC: 25-55% cholesterol; and 0.5-15% PEG-DMG. In one embodiment, the lipid nanoparticle comprises a molar ratio of about 50% Compound 25: about 10% DSPC: about 38.5% cholesterol: about 1.5% PEG-DMG (i.e., Compound 25:DSPC:cholesterol:PEG-DMG at about a 50:10:38.5:1.5 ratio). In one embodiment, the lipid nanoparticle comprises a molar ratio of 50% Compound 25: 10% DSPC: 38.5% cholesterol: 1.5% PEG-DMG (i.e., Compound 25:DSPC:cholesterol:PEG-DMG at a 50:10:38.5:1.5 ratio).

In some aspects, the disclosure provides a drug product, such as a vaccine, comprising any of the foregoing or related lipid nanoparticle carriers for use in therapy, for example, prophylactic or therapeutic treatment (e.g., cancer therapy), optionally with instructions for use in such therapy.

In some aspects related to the foregoing drug product or vaccine, the disclosure provides a first lipid nanoparticle carrier comprising a pharmaceutical composition, wherein the pharmaceutical composition comprises: an mRNA having an open reading frame encoding at least one first antigen of interest (e.g., at least one cancer antigen, viral antigen, bacterial antigen); an mRNA having an open reading frame encoding a constitutively active human STING polypeptide; and a pharmaceutically acceptable carrier or excipient.

In some aspects, the disclosure provides a second lipid nanoparticle carrier comprising a pharmaceutical composition, wherein the pharmaceutical composition comprises: an mRNA having an open reading frame encoding at least one second antigen of interest (e.g., at least one cancer antigen, viral antigen, bacterial antigen); an mRNA having an open reading frame encoding a constitutively active human STING polypeptide; and a pharmaceutically acceptable carrier or excipient.

In some aspects, the disclosure provides a third lipid nanoparticle carrier comprising a pharmaceutical composition, wherein the pharmaceutical composition comprises: an mRNAs having an open reading frame encoding at least one third antigen of interest (e.g., at least one cancer antigen, viral antigen, bacterial antigen); an mRNA having an open reading frame encoding a constitutively active human STING polypeptide; and a pharmaceutically acceptable carrier or excipient.

In some aspects, the disclosure provides a fourth lipid nanoparticle carrier comprising a pharmaceutical composition, wherein the pharmaceutical composition comprises: an mRNAs having an open reading frame encoding at least one fourth antigen of interest (e.g., at least one (e.g., cancer antigen, viral antigen, bacterial antigen); an mRNA having an open reading frame encoding a constitutively active human STING polypeptide; and a pharmaceutically acceptable carrier or excipient.

In other aspects, the disclosure provides a first lipid nanoparticle carrier comprising a pharmaceutical composition, wherein the pharmaceutical composition comprises: an mRNA having an open reading frame encoding at least one HPV antigen (e.g., at least one E6 antigen and/or at least one E7 antigen); an mRNA having an open reading frame encoding a constitutively active human STING polypeptide; and a pharmaceutically acceptable carrier or excipient.

In some aspects, the disclosure provides a second lipid nanoparticle carrier comprising a pharmaceutical composition, wherein the pharmaceutical composition comprises: an mRNA having an open reading frame encoding at least one second HPV antigen (e.g., at least one E6 antigen and/or at least one E7 antigen); an mRNA having an open reading frame encoding a constitutively active human STING polypeptide; and a pharmaceutically acceptable carrier or excipient.

In some aspects, the disclosure provides a third lipid nanoparticle carrier comprising a pharmaceutical composition, wherein the pharmaceutical composition comprises: an mRNAs having an open reading frame encoding at least one third HPV antigen (e.g., at least one E6 antigen and/or at least one E7 antigen); an mRNA having an open reading frame encoding a constitutively active human STING polypeptide; and a pharmaceutically acceptable carrier or excipient.

In some aspects, the disclosure provides a fourth lipid nanoparticle carrier comprising a pharmaceutical composition, wherein the pharmaceutical composition comprises: an mRNAs having an open reading frame encoding at least one fourth HPV antigen (e.g., at least one E6 antigen and/or at least one E7 antigen); an mRNA having an open reading frame encoding a constitutively active human STING polypeptide; and a pharmaceutically acceptable carrier or excipient.

In some aspects of the foregoing drug product or vaccine, each of the first, second, third and fourth lipid nanoparticle carriers, comprises a peptide antigen comprising 20, 21, 22, 23, 24, or 25 amino acids in length. In some aspects, each peptide antigen comprises 25 amino acids in length.

In some aspects of the foregoing first, second, third and fourth lipid nanoparticle carriers, wherein the HPV antigen(s) comprises one or more of the amino acid sequences set forth in SEQ ID NOs: 36-72. In some aspects, the HPV antigen(s) comprises one or more of the amino acid sequences set forth in SEQ ID NOs: 73-94.

In some aspects of the foregoing first, second, third and fourth lipid nanoparticle carriers, the constitutively active human STING polypeptide comprises mutation V155M. In some aspects, the constitutively active human STING polypeptide comprises the amino acid sequence shown in SEQ ID NO: 1. In some aspects, the constitutively active human STING polypeptide comprises a 3′ UTR comprising at least one miR-122 microRNA binding site. In some aspects, the mRNA encoding the constitutively active human STING polypeptide comprises the nucleotide sequence shown in SEQ ID NO: 199, 1319 or 1320.

In some aspects, the disclosure provides a drug product, such as a vaccine, comprising any of the foregoing or related lipid nanoparticle carriers for use in prophylactic or therapeutic treatment (e.g., cancer therapy), optionally with instructions for use in therapy. In some aspects, the disclosure provides a drug product, such as a vaccine, comprising any of the foregoing first, second, third and fourth lipid nanoparticle carriers, for use in cancer therapy, optionally with instructions for use in cancer therapy.

In some aspects, the disclosure provides a drug product, such as a vaccine, comprising a first, second, third and fourth lipid nanoparticle carriers, for use in prophylactic or therapeutic treatment (e.g., cancer therapy), optionally with instructions for use in therapy, wherein:
(i) the first lipid nanoparticle carrier comprises a pharmaceutical composition, wherein the pharmaceutical composition comprises: an mRNA having an open reading frame encoding at least one first antigen of interest (e.g., at least one cancer antigen, viral antigen, bacterial antigen, for example, at least one E6 antigen and/or at least one E7 antigen); an mRNA having an open reading frame encoding a constitutively active human STING polypeptide; and a pharmaceutically acceptable carrier or excipient;
(ii) the second lipid nanoparticle carrier comprises a pharmaceutical composition, wherein the pharmaceutical composition comprises: an mRNA having an open reading frame encoding at least one second antigen of interest (e.g., cancer antigen, viral antigen, bacterial antigen, for example, at least one E6 antigen and/or at least one E7 antigen); an mRNA having an open reading frame encoding a constitutively active human STING polypeptide; and a pharmaceutically acceptable carrier or excipient;
(iii) the third lipid nanoparticle carrier comprises a pharmaceutical composition, wherein the pharmaceutical composition comprises: an mRNA having an open reading frame encoding at least one third antigen of interest (e.g., cancer antigen, viral antigen, bacterial antigen, for example, at least one E6 antigen and/or at least one E7 antigen); an mRNA having an open reading frame encoding a constitutively active human STING polypeptide; and a pharmaceutically acceptable carrier or excipient; and
(iv) the fourth lipid nanoparticle carrier comprises a pharmaceutical composition, wherein the pharmaceutical composition comprises: an mRNA having an open reading frame encoding at least one fourth antigen of interest (e.g., cancer antigen, viral antigen, bacterial antigen, for example, at least one E6 antigen and/or at least one E7 antigen); an mRNA having an open reading frame encoding a constitutively active human STING polypeptide; and a pharmaceutically acceptable carrier or excipient.

In any of the foregoing or related aspects, the disclosure provides a method for treating a subject, comprising: administering to a subject in need thereof any of the foregoing or related immunomodulatory therapeutic compositions or any of the foregoing or related lipid nanoparticle carriers. In some aspects, the immunomodulatory therapeutic composition or lipid nanoparticle carrier is administered in combination with another therapeutic agent (e.g., a cancer therapeutic agent). In some aspects, the immunomodulatory therapeutic composition or lipid nanoparticle carrier is administered in combination with an inhibitory checkpoint polypeptide. In some aspects, the inhibitory checkpoint polypeptide is an antibody or fragment thereof that specifically binds to a molecule selected from the group consisting of PD-1, PD-L1, TIM-3, VISTA, A2AR, B7-H3, B7-H4, BTLA, CTLA-4, IDO, KIR and LAG3.

In some aspects, the disclosure provides a composition (e.g., a vaccine) comprising an mRNA encoding an antigen of interest and an mRNA encoding a polypeptide that enhances an immune response to the antigen of interest (e.g., immune potentiator, e.g., STING polypeptide) wherein the mRNA encoding the antigen of interest (Ag) and the mRNA encoding the polypeptide that enhances an immune response to the antigen of interest (e.g., immune potentiator (IP), e.g., STING polypeptide) are formulated at an Ag:IP mass ratio of 1:1, 2:1, 3:1, 4:1, 5:1, 6:1, 7:1, 8:1, 9:1, 10:1 or 20:1. Alternatively, the IP:Ag mass ratio can be, for example: 1:1, 1:2, 1:3, 1:4, 1:5, 1:6, 1:7, 1:8, 1:9, 1:10 or 1:20. In some aspects, the composition is formulated at a mass ratio of 5:1 of mRNA encoding the antigen of interest to the mRNA encoding the polypeptide that enhances an immune to the antigen of interest (e.g., immune potentiator, e.g., STING polypeptide) (i.e., Ag:IP ratio of 5:1 or, alternatively, IP:Ag ratio of 1:5). In some aspects, the composition is formulated at a mass ratio of 10:1 of mRNA encoding the antigen of interest to the mRNA encoding the polypeptide that enhances an immune to the antigen of interest (e.g., immune potentiator, e.g., STING polypeptide) (i.e., Ag:IP ratio of 10:1 or, alternatively, IP:Ag ratio of 1:10).

In another aspect, the disclosure pertains to a method for enhancing an immune response to an antigen(s) of interest, the method comprising administering to a subject in need thereof a mmRNA composition of disclosure encoding an antigen(s) of interest and a polypeptide that enhances an immune response to the antigen(s) of interest, or lipid nanoparticle thereof, or pharmaceutical composition thereof, such that an immune response to the antigen of interest is enhanced in the subject. In one aspect, enhancing an immune response in a subject comprises stimulating cytokine production (e.g., IFN-γ or TNF-α). In another aspect, enhancing an immune response in a subject comprises stimulating antigen-specific CD8⁺ T cell activity, e.g., priming, proliferation and/or survival (e.g., increasing the effector/memory T cell population). In one aspect, enhancing an immune response in a subject comprises stimulating antigen-specific CD4⁺ T cell activity (e.g., increasing helper T cell activity). In other aspects, enhancing an immune response in a subject comprises stimulating B cell responses (e.g., increasing antibody production).

In some aspects, enhancing an immune response in a subject comprises stimulating cytokine production, stimulating antigen-specific CD8⁺ T cell responses, stimulating antigen-specific CD4⁺ helper cell responses, increasing the effector memory CD62L1^{lo} T cell population, stimulating B cell activity or stimulating antigen-specific antibody production, or any combination of the foregoing responses.

In some aspects, the enhanced immune response comprises stimulating cytokine production, wherein the cytokine is IFN-γ or TNF-α, or both. In some aspects, the enhanced immune response comprises stimulating antigen-specific CD8⁺ T cell responses, wherein the antigen-specific CD8⁺ T cell response comprises CD8⁺ T cell proliferation or CD8⁺ T cell cytokine production or both. In some aspects, CD8⁺ T cell cytokine production increases by at least 5% or at least 10% or at least 15% or at least 20% or at least 25% or at least 30% or at least 35% or at least 40% or at least 45% or at least 50%.

In some aspects, the enhanced immune response comprises an antigen-specific CD8⁺ T cell response, wherein the CD8⁺ T cell response comprises CD8⁺ T cell proliferation, and wherein the percentage of CD8⁺ T cells among the total T cell population increases by at least 5% or at least 10% or at least 15% or at least 20% or at least 25% or at least 30% or at least 35% or at least 40% or at least 45% or at least 50%.

In some aspects, the enhanced immune response comprises an antigen-specific CD8⁺ T cell response, wherein the CD8⁺ T cell response comprises an increase in the percentage of effector memory CD62L1^{lo} T cells among CD8⁺ T cells.

In another aspect, the disclosure pertains to a method for enhancing an immune response to an antigen(s) of interest, the method comprising administering to a subject in need thereof an mRNA composition of disclosure encoding an antigen(s) of interest and a polypeptide that enhances an immune response to the antigen(s) of interest, or lipid nanoparticle thereof, or pharmaceutical composition thereof, such that an immune response to the antigen of interest is enhanced in the subject, wherein the immune response to the antigen of interest is maintained for greater than 10 days, for greater than 15 days, for greater than 20 days, for greater than 25 days, for greater than 30 days, for greater than 40 days, for greater than 50 days, for greater than 60 days, for greater than 70 days, for greater than 80 days, for greater than 90 days, greater than 100, 120, 150, 200, 250, 300 days or 1 year or more.

In one aspect, the disclosure provides methods for enhancing an immune response to an antigen(s) of interest, wherein the subject is administered two different immune potentiator mRNA (e.g., mmRNA) constructs (wherein one or both constructs also encode, or are administered with an mRNA (e.g., mmRNA) construct that encodes, the antigen(s) of interest), either at the same time or sequentially. In one aspect, the subject is administered an immune potentiator mRNA composition that stimulates dendritic cell development or activity prior to administering to the subject an immune potentiator mmRNA composition that stimulates Type I interferon pathway signaling.

In other aspects, the disclosure provides methods of stimulating an immune response to a tumor in a subject in need thereof, wherein the method comprises administering to the subject an effective amount of a composition comprising at least one mRNA construct encoding a tumor antigen(s) and an mRNA construct encoding a polypeptide that enhances an immune response to the tumor antigen(s), or a lipid nanoparticle thereof, or a pharmaceutical composition thereof, such that an immune response to the tumor is stimulated in the subject. In one aspect, the tumor is a liver cancer, a colorectal cancer, a pancreatic cancer, a non-small cell lung cancer (NSCLC), a melanoma cancer, a cervical cancer or a head or neck cancer. In some aspects, the subject is a human.

In one embodiment, the disclosure provides a method of preventing or treating an Human Papilloma Virus (HPV)-associated cancer in a subject in need thereof, the method comprising administering to the subject a composition comprising at least one mRNA construct encoding:(i) at least one HPV antigen of interest and (ii) a polypeptide that enhances an immune response against the at least one HPV antigen of interest, such that an immune response to the at least one HPV antigen of interest is enhanced. In one embodiment, the polypeptide that enhances an immune response against the at least one HPV antigen(s) of interest is a STING polypeptide. In one embodiment, the at least one HPV antigen is at least one E6 antigen, at least one E7 antigen or a combination of at least one E6 antigen and at least one E7 antigen (e.g, soluble or intracellular forms of E6 and/or E7). In one embodiment, the at least one HPV antigen and the polypeptide are encoded on separate mRNAs and are coformulated in a lipid nanoparticular prior to administration to the subject. Alternatively, the HPV antigen(s) and polypeptide can be encoded on the same mRNA. In one embodiment, the subject is at risk for exposure to HPV and the composition is administered prior to exposure to HPV. In another embodiment, the subject is infected with HPV or has an HPV-associated cancer. In one embodiment, the HPV-associated cancer is selected from the group consisting of cervical, penile, vaginal, vulval, anal and oropharyngeal cancers. In one embodiment, the subject with cancer is also treated with an immune checkpoint inhibitor.

In another aspect, the disclosure provides methods of stimulating an immune response to a pathogen in a subject in need thereof, wherein the method comprises administering to the subject an effective amount of a composition comprising at least one mRNA construct encoding a pathogen antigen(s) and an mRNA construct encoding a polypeptide that enhances an immune response to the pathogen antigen(s), or a lipid nanoparticle thereof, or a pharmaceutical composition thereof, such that an immune response to the pathogen is stimulated in the subject. In one aspect, the pathogen is selected from the group consisting of viruses, bacteria, protozoa, fungi and parasites. In one aspect, the pathogen is a virus, such as a human papillomavirus (HPV). In one aspect, the pathogen is a bacteria. In one aspect, the subject is a human.

In any of the foregoing or related aspects, the disclosure provides a pharmaceutical composition comprising the lipid nanoparticle, and a pharmaceutically acceptable carrier. In some aspects, the pharmaceutical composition is formulated for intramuscular delivery.

In any of the foregoing or related aspects, the disclosure provides a lipid nanoparticle, and an optional pharmaceutically acceptable carrier, or a pharmaceutical composition for use in enhancing an immune response in an individual (e.g., treating or delaying progression of cancer in an individual), wherein the treatment comprises administration of the composition in combination with a second composition, wherein the second composition comprises a checkpoint inhibitor polypeptide and an optional pharmaceutically acceptable carrier.

In any of the foregoing or related aspects, the disclosure provides use of a lipid nanoparticle, and an optional pharmaceutically acceptable carrier, in the manufacture of a medicament for enhancing an immune response in an individual (e.g., treating or delaying progression of cancer in an individual), wherein the medicament comprises the lipid nanoparticle and an optional pharmaceutically acceptable carrier and wherein the treatment comprises administration of the medicament, optionally in combination with a composition comprising a checkpoint inhibitor polypeptide and an optional pharmaceutically acceptable carrier.

In any of the foregoing or related aspects, the disclosure provides a kit comprising a container comprising a lipid nanoparticle, and an optional pharmaceutically acceptable carrier, or a pharmaceutical composition, and a package insert comprising instructions for administration of the lipid nanoparticle or pharmaceutical composition for enhancing an immune response in an individual (e.g., treating or delaying progression of cancer in an individual). In some aspects, the package insert further comprises instructions for administration of the lipid nanoparticle or pharmaceutical composition alone, or in combination with a composition comprising a checkpoint inhibitor polypeptide and an optional pharmaceutically acceptable carrier for enhancing an immune response in an individual (e.g., treating or delaying progression of cancer in an individual).

In any of the foregoing or related aspects, the disclosure provides a kit comprising a medicament comprising a lipid nanoparticle, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier for enhancing an immune response in an individual (e.g., treating or delaying progression of cancer in an individual). In some aspects, the kit further comprises a package insert comprising instructions for administration of the first medicament prior to, current with, or subsequent to administration of the second medicament for enhancing an immune response in an individual (e.g., treating or delaying progression of cancer in an individual).

In any of the foregoing or related aspects, the disclosure provides a lipid nanoparticle, a composition, or the use thereof, or a kit comprising a lipid nanoparticle or a composition as described herein, wherein the checkpoint inhibitor polypeptide inhibits PD1, PD-L1, CTLA4, or a combination thereof. In some aspects, the checkpoint inhibitor polypeptide is an antibody. In some aspects, the checkpoint inhibitor polypeptide is an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, and a combination thereof. In some aspects, the checkpoint inhibitor polypeptide is an anti-PD-L1 antibody selected from atezolizumab, avelumab, or durvalumab. In some aspects, the checkpoint inhibitor polypeptide is an anti-CTLA-4 antibody selected from tremelimumab or ipilimumab. In some aspects, the checkpoint inhibitor polypeptide is an anti-PD1 antibody selected from nivolumab or pembrolizumab.

In related aspects, the disclosure provides a method of reducing or decreasing a size of a tumor or inhibiting a tumor growth in a subject in need thereof comprising administering to the subject any of the foregoing or related lipid nanoparticles of the disclosure, or any of the foregoing or related compositions of the disclosure.

In related aspects, the disclosure provides a method inducing an anti-tumor response in a subject with cancer comprising administering to the subject any of the foregoing or related lipid nanoparticles of the disclosure, or any of the foregoing or related compositions of the disclosure. In some aspects, the anti-tumor response comprises a T-cell response. In some aspects, the T-cell response comprises CD8+ T cells.

In some aspects of the foregoing methods, the composition is administered by intramuscular injection.

In some aspects of the foregoing methods, the method further comprises administering a second composition comprising a checkpoint inhibitor polypeptide, and an optional pharmaceutically acceptable carrier. In some aspects, the checkpoint inhibitor polypeptide inhibits PD1, PD-L1, CTLA4, or a combination thereof. In some aspects, the checkpoint inhibitor polypeptide is an antibody. In some aspects, the checkpoint inhibitor polypeptide is an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, and a combination thereof. In some aspects, the checkpoint inhibitor polypeptide is an anti-PD-L1 antibody selected from atezolizumab, avelumab, or durvalumab. In some aspects, the checkpoint inhibitor polypeptide is an anti-CTLA-4 antibody selected from tremelimumab or ipilimumab. In some aspects, the checkpoint inhibitor polypeptide is an anti-PD1 antibody selected from nivolumab or pembrolizumab.

In some aspects of any of the foregoing or related methods, the composition comprising the checkpoint inhibitor polypeptide is administered by intravenous injection. In some aspects, the composition comprising the checkpoint inhibitor polypeptide is administered once every 2 to 3 weeks. In some aspects, the composition comprising the checkpoint inhibitor polypeptide is administered once every 2 weeks or once every 3 weeks.

In some aspects, the composition comprising the checkpoint inhibitor polypeptide is administered prior to, concurrent with, or subsequent to administration of the lipid nanoparticle or pharmaceutical composition thereof.

### BRIEF DESCRIPTION OF THE DRAWINGS

FIG. 1 is a bar graph showing stimulation of IFN-β production in TF1a cells transfected with constitutively active STING mRNA constructs.

FIG. 2 is a bar graph showing activation of an interferon-sensitive response element (ISRE) by constitutively active STING constructs. STING variants 23a and 23b correspond to SEQ ID NO: 1, STING variant 42 corresponds to SEQ ID NO: 2, STING variants 19, 21a and 21b correspond to SEQ ID NO: 3, STING variant 41 corresponds to SEQ ID NO: 4, STING variant 43 corresponds to SEQ ID NO: 5, STING variant 45 corresponds to SEQ ID NO: 6, STING variant 46 corresponds to SEQ ID NO: 7, STING variant 47 corresponds to SEQ ID NO: 8, STING variant 56 corresponds to SEQ ID NO: 9 and STING variant 57 corresponds to SEQ ID NO: 10.

FIGS. 3A-3B are bar graphs showing activation of an interferon-sensitive response element (ISRE) by constitutively active IRF3 constructs (FIG. 3A) or constitutively active IRF7 constructs (FIG. 3B). IRF3 variants 1, 3 and 4 correspond to SEQ ID NO: 12 and IRF3 variants 2 and 5 correspond to SEQ ID NO: 11 (variants have different tags). IRF7 variant 36 corresponds to SEQ ID NO: 18 and variant 31 is the murine version of SEQ ID NO: 18. IRF7 variant 32 corresponds to SEQ ID NO: 17 and IRF7 variant 33 corresponds to SEQ ID NO: 14.

FIG. 4 is a bar graph showing activation of an NFκB-luciferase reporter gene by constitutively active cFLIP and IKKβ mRNA constructs.

FIG. 5 is a graph showing activation of an NFκB-luciferase reporter gene by constitutively active RIPK1 mRNA constructs.

FIG. 6 is a bar graph showing TNF-α induction in SKOV3 cells transfected with DIABLO mRNA constructs.

FIG. 7 is a bar graph showing interleukin 6 (IL-6) induction in SKOV3 cells transfected with DIABLO mRNA constructs.

FIGS. 8A-8B are graphs showing intracellular staining (ICS) of CD8⁺ splenocytes from mice immunized with HPV E6/E7 vaccine constructs coformulated with either a STING, IRF3 or IRF7 immune potentiator mRNA construct on day 21 post first immunization. FIG. 8A shows E7-specific responses for IFN-γ ICS. FIG. 8B shows E7-specific responses for TNF-α ICS.

FIGS. 9A-9B are graphs showing intracellular staining (ICS) of CD8⁺ splenocytes from mice immunized with HPV E6/E7 vaccine constructs coformulated with either a STING, IRF3 or IRF7 immune potentiator mRNA construct. FIG. 9A shows E6-specific responses for IFN-γ ICS. FIG. 9B shows 67-specific responses for TNF-α ICS.

FIGS. 10A-10B are graphs showing E7-specific responses for IFN-γ intracellular staining (ICS) of day 21 (FIG. 10A) or day 53 (FIG. 10B) CD8⁺ splenocytes from mice immunized with HPV E6/E7 vaccine constructs coformulated with either a STING, IRF3 or IRF7 immune potentiator mRNA construct.

FIGS. 11A-11B are graphs showing intracellular staining (ICS) of CD8⁺ splenocytes for IFN-γ on days 21 and 53 from mice immunized with HPV E6/E7 vaccine constructs coformulated with either a STING, IRF3 or IRF7 immune potentiator mRNA construct. FIG. 11A shows E7-specific responses from mice immunized with intracellular E6/E7. FIG. 11B shows E7-specific responses from mice immunized with soluble E6/E7.

FIGS. 12A-12B are graphs showing the percentage of CD8b⁺ cells among the live CD45+ cells for day 21 (FIG. 12A) or day 53 (FIG. 12B) spleen cells from mice immunized with HPV E6/E7 vaccine constructs coformulated with either a STING, IRF3 or IRF7 immune potentiator mRNA construct.

FIGS. 13A-13B are graphs showing E7-MHC1-tetramer (specific for the epitope RAHYNIVTF) staining of day 21 (FIG. 13A) or day 53 (FIG. 13B) CD8b⁺ splenocytes from mice immunized with HPV E6/E7 vaccine constructs coformulated with either a STING, IRF3 or IRF7 immune potentiator mRNA construct.

FIGS. 14A-14D are graphs showing that the majority of E7-tetramer⁺ CD8⁺ cells have an “effector memory” CD62L^{lo} phenotype, with comparison of day 21 versus day 53 E7-tetramer+ CD8 cells demonstrating that this “effector-memory” CD62L^{lo} phenotype was maintained throughout the study. FIGS. 14A (day 21) and 14B (day 53) show increased % of CD8 with effector memory CD62Llo phenotype. FIGS. 14C and 14D show increased % of E7-tetramer+CD8 are CD62Llo, when mice were immunized with HPV E6/E7 vaccine constructs coformulated with either a STING, IRF3 or IRF7 immune potentiator mRNA construct.

FIGS. 15A-15B are graphs showing MC38 neoantigen-specific responses by IFN-□ intracellular staining (ICS) of day 21 (FIG. 15A) or day 35 (FIG. 15B) CD8⁺ splenocytes from mice immunized with MC38 neo-antigen vaccine construct (ADRvax) coformulated with either a STING, IRF3 or IRF7 immune potentiator mRNA construct.

FIGS. 16A-16B are graphs showing the percentage of CD8b⁺ cells among live CD45⁺ cells in spleen or PBMCs (FIG. 16A) or the percentage of CD62L^{lo} cells among CD8b⁺ cell in spleen or PBMCs (FIG. 16B) from mice immunized with MC38 neoantigen vaccine construct (ADRvax) coformulated with either a STING, IRF3 or IRF7 immune potentiator mRNA construct.

FIG. 17 is a graph showing antibody titer comparisons from mice treated with the indicated bacterial antigen mRNA constructs alone (at 0.2 μg) or treated with the bacterial peptide mRNA construct coformulated with a STING immune potentiator mRNA construct.

FIG. 18 depicts NRAS and KRAS mutation frequency in colorectal cancer as identified using cBioPortal.

FIGS. 19A-19C are graphs showing tumor volume from mice treated prophylactically as indicated with HPV E6/E7 construct together with a STING immune potentiator mRNA construct (alone or in combination with anti-CTLA-4 or anti-PD1 treatment on day 6, 9, and 12), either prior to or at the time of challenge with a TC1 tumor that expresses HPV E7, showing inhibition of tumor growth by the HPV E6/E7+STING treatment. Certain mice were treated on days −14 and −7 with soluble E6/E7+STING (FIG. 19A) or with intracellular E6/E7+STING (FIG. 19B), with tumor challenge on day 1. Other mice were treated on days 1 and 8 with soluble E6/E7+STING (FIG. 19C), with tumor challenge on day 1.

FIGS. 20A-20I are graphs showing tumor volume from mice treated therapeutically as indicated with HPV E6/E7 construct together with a STING immune potentiator mRNA construct (FIG. 20A), alone or in combination with anti-CTLA-4 treatment on day 13, 16 and 19 (FIG. 20B) or anti-PD1 treatment on day 13, 16 and 19 (FIG. 20C), after challenge with a TC1 tumor that expresses HPV E7, showing inhibition of tumor growth by the HPV E6/E7+STING treatment. FIGS. 20D-20I show treatments with murine STING ligand DMXAA.

FIG. 21 provides graphs showing tumor volume from mice treated therapeutically as indicated with HPV E6/E7 construct together with a STING immune potentiator mRNA construct in mice bearing tumors of 200 mm³ volume size (upper graphs) or 300 mm³ volume size (lower graphs).

FIG. 22 is a graph showing intracellular staining (ICS) of CD8⁺ splenocytes for IFN-γ from mice immunized with an ADR vaccine construct coformulated with a STING immune potentiator at the indicated Ag:STING ratios on day 21 post first immunization. CD8+ cells were restimulated with either the mutant ADR antigen composition (comprising three peptides) or the wild-type ADR composition (as a control).

FIG. 23 is a graph showing intracellular staining (ICS) of CD8⁺ splenocytes for TNF-α from mice immunized with an ADR vaccine construct coformulated with a STING immune potentiator at the indicated Ag:STING ratios on day 21 post first immunization. CD8+ cells were restimulated with either the mutant ADR antigen composition (comprising three peptides) or the wild-type ADR composition (as a control).

FIGS. 24A-24C are graphs showing intracellular staining (ICS) of CD8⁺ splenocytes for IFN-γ from mice immunized with an ADR vaccine construct coformulated with a STING immune potentiator at the indicated Ag:STING ratios on day 21 post first immunization. CD8+ cells were restimulated with either a mutant or wild-type (as a control) peptide contained within the ADR antigen composition. FIG. 24A shows responses to the Adpk1 peptide within the ADR composition. FIG. 24B shows the response to the Reps1 peptide within the ADR composition. FIG. 24C shows the response to the Dpagt1 peptide within the ADR composition.

FIG. 25 is a graph showing antigen-specific T cell responses to MHC class I epitopes within the CA-132 vaccine, as measured by ELISpot analysis for IFN-γ, from mice treated with a coformulation of CA-132 and STING immune potentiator, at the indicated different Ag:STING ratios.

FIG. 26 is a bar graph showing antigen-specific T cell responses to MHC class I epitopes within the CA-132 vaccine, following restimulation with the CA-87 peptide, as measured by ELISpot analysis for IFN-γ, from mice treated with a coformulation of CA-132 and STING immune potentiator, at the indicated different Ag:STING ratios.

FIG. 27 is a graph showing intracellular staining (ICS) of CD8⁺ splenocytes for IFN-γ from mice immunized with an HPV16 E7 vaccine construct coformulated with a STING immune potentiator at the indicated Ag:STING ratios on day 21 post first immunization.

FIGS. 28A-28C are bar graphs showing TNF I intracellular staining (ICS) results for CD8+ T cells from cynomolgus monkeys treated with HPV vaccine+STING constructs, followed by ex vivo stimulation with either HPV16 E6 peptide pool (FIG. 28A), HPV16 E7 peptide pool (FIG. 28B) or medium (negative control) (FIG. 28C).

FIGS. 29A-29C are bar graphs showing IL-2 intracellular staining (ICS) results for CD8+ T cells from cynomolgus monkeys treated with HPV vaccine+STING constructs, followed by ex vivo stimulation with either HPV16 E6 peptides (FIG. 29A), HPV16 E7 peptides (FIG. 29B) or medium (negative control) (FIG. 29C).

FIG. 30 is a graph showing ELISA results for anti-E6 IgG in serum from cynomolgus monkeys treated with HPV vaccine+STING constructs.

FIG. 31 is a graph showing ELISA results for anti-E7 IgG in serum from cynomolgus monkeys treated with HPV vaccine+STING constructs.

FIG. 32 is a graph showing the intracellular staining (ICS) results for CD8+ splenocytes for IFN-γ from mice immunized with mutant KRAS vaccine+STING construct followed by ex vivo stimulation with KRAS-G12V peptide.

FIG. 33 is a graph showing the intracellular staining (ICS) results for CD8+ splenocytes for IFN-γ from mice immunized with mutant KRAS vaccine+STING construct followed by ex vivo stimulation with KRAS-G12D peptide.

FIG. 34 is a graph showing the intracellular staining (ICS) results or CD8+ splenocytes for IFN-γ from mice immunized with mutant KRAS vaccine+STING construct followed by ex vivo co-culture with Cos7-All cells pulsed with KRAS-G12V.

FIG. 35 is a graph showing the intracellular staining (ICS) results or CD8+ splenocytes for IFN-γ from mice immunized with mutant KRAS vaccine+STING construct followed by ex vivo co-culture with Cos7-All cells pulsed with KRAS-G12D.

FIG. 36 is a graph showing the intracellular staining (ICS) results or CD8+ splenocytes for IFN-γ from mice immunized with an All viral epitope concatemer with STING or with nontranslatable mRNA control (NTFIX) constructs followed by ex vivo stimulation with individual viral epitopes.

FIGS. 37A-37B are graphs showing intracellular staining (ICS) of CD8+ splenocytes from mice immunized with HPV vaccine constructs coformulated with either STING, IRF3/IRF7 or IRF3/IRF7/IKKβ immune potentiator mRNA constructs on day 21 post first immunization. FIG. 37A shows E7-specific responses for IFN—Y ICS. FIG. 37B shows E7-specific responses for TNF-α ICS.

FIGS. 38A-38C are graphs showing intracellular staining (ICS) of CD8+ splenocytes from mice immunized with OVA antigen coformulated with either STING, TAK1, TRAM or MyD88 immune potentiator mRNA constructs on day 25 post first immunization. FIG. 38A shows OVA-specific responses for IFN-γ ICS. FIG. 38B shows OVA-specific responses for TNF-α ICS. FIG. 38C shows OVA-specific responses for IL-2 ICS.

FIG. 39 is a bar graph showing intracellular staining (ICS) of CD8+ splenocytes for IFN-γ from mice immunized with OVA antigen coformulated with either STING, MAVS, IKKβ, Caspase 1+Caspase 4+IKKβ, MLKL or MLKL+STING immune potentiator mRNA constructs on day 21 post first immunization. DMXAA, a chemical activator of STING, was used as a comparator.

FIG. 40 is a bar graph showing intracellular staining (ICS) of CD8+ splenocytes for IFN-γ from mice immunized with OVA antigen coformulated with either STING, MAVS, IKKβ, Caspase 1+Caspase 4+IKKβ, MLKL or MLKL+STING immune potentiator mRNA constructs on day 50 post first immunization. DMXAA, a chemical activator of STING, was used as a comparator.

FIGS. 41A-41B are bar graphs showing intracellular staining (ICS) of CD8+ splenocytes for IFN-γ from mice immunized with OVA antigen coformulated or coadministered with the indicated constitutively active STING mutant constructs. FIG. 41A shows day 21 post immunization. FIG. 41B shows day 90 post first immunization.

FIGS. 42A-42B are bar graphs showing intracellular staining (ICS) of CD8+ splenocytes for IFN-γ from CD4-depleted mice immunized with HPV vaccine constructs coformulated with a STING immune potentiator mRNA construct. FIG. 42A shows day 21 post first immunization. FIG. 42B shows day 50 post first immunization.

FIG. 43 provides graphs showing tumor volume in mice bearing TC1 HPV tumors treated with an HPV-STING vaccine either alone or in combination with anti-CD4 (to deplete CD4 T cells) or anti-CD8 (to deplete CD8 T cells).

FIGS. 44A-44B are graphs showing the percentage of CD62L^{lo} cells among CD4^{hi}CD8⁺ cells from spleens of mice immunized with MC38 antigen vaccine construct coformulated with a STING immune potentiator mRNA construct at the indicated Ag and STING dosages. FIG. 44A shows results for day 21 spleen cells. FIG. 44B shows the results for day 54 spleen cells.

FIG. 45 is a bar graph showing antigen-specific IFN-γ T cell responses from mice immunized with mRNA encoding a concatemeric of 20 murine epitopes (CA-132) in combination with a STING immunopotentiator mRNA, as compared to standard adjuvants, or unformulated (not encapsulated in LNP). Data shown is for in vitro peptide restimulation with Class II epitopes (CA-82 and CA-83) encoded within the concatemer.

FIG. 46 is a bar graph showing antigen-specific IFN-γ T cell responses from mice immunized with mRNA encoding a concatemeric of 20 murine epitopes (CA-132) in combination with a STING immunopotentiator mRNA, as compared to standard adjuvants, or unformulated (not encapsulated in LNP). Data shown is for in vitro peptide restimulation with Class I epitopes (CA-87, CA-90 and CA-93) encoded within the concatemer.

FIG. 47 is a bar graph showing antigen-specific IFN-γ T cell responses from mice immunized with mRNA encoding a concatemeric of 20 murine epitopes (CA-132) in combination with a STING immunopotentiator mRNA, wherein the STING construct was administered either simultaneously with the vaccine, 24 hours later or 48 hours later. Data shown is for in vitro peptide restimulation with either Class II epitopes (CA-82 and CA-83) or Class I epitopes (CA-87, CA-90 and CA-93) encoded within the concatemer.

FIG. 48 shows antigen-specific responses from mice immunized with mRNA encoding a concatemeric of 52 murine epitopes in combination with a STING immunopotentiator mRNA at varying Ag and STING dosages and Ag:STING ratios. Data shown is for in vitro restimulation with the peptide sequence corresponding to the Class II epitope CA-82, encoded within the concatemer.

FIG. 49 shows antigen-specific responses from mice immunized with mRNA encoding a concatemeric of 52 murine epitopes in combination with a STING immunopotentiator mRNA at varying Ag and STING dosages and Ag:STING ratios. Data shown is for in vitro restimulation with the peptide sequence corresponding to the Class II epitope CA-83, encoded within the concatemer.

FIG. 50 shows antigen-specific responses from mice immunized with mRNA encoding a concatemeric of 52 murine epitopes in combination with a STING immunopotentiator mRNA at varying Ag and STING dosages and Ag:STING ratios. Data shown is for in vitro restimulation with the peptide sequence corresponding to Class I epitope CA-87, encoded within the concatemer.

FIG. 51 shows antigen-specific responses from mice immunized with mRNA encoding a concatemeric of 52 murine epitopes in combination with a STING immunopotentiator mRNA at varying Ag and STING dosages and Ag:STING ratios. Data shown is for in vitro restimulation with the peptide sequence corresponding to Class I epitope CA-93, encoded within the concatemer.

FIG. 52 shows antigen-specific responses from mice immunized with mRNA encoding a concatemeric of 52 murine epitopes in combination with a STING immunopotentiator mRNA at varying Ag and STING dosages and Ag:STING ratios. Data shown is for in vitro restimulation with the peptide sequence corresponding to Class I epitope CA-113, encoded within the concatemer.

FIG. 53 shows antigen-specific responses from mice immunized with mRNA encoding a concatemeric of 52 murine epitopes in combination with a STING immunopotentiator mRNA at varying Ag and STING dosages and Ag:STING ratios. Data shown is for in vitro restimulation with the peptide sequence corresponding to Class II epitope CA-90, encoded within the concatemer.

FIG. 54 is a bar graph showing cell viability of Hep3B cells transfected with MLKL 1-180 mRNA constructs, as measured using the CellTiter-Glo® Luminescent Cell Viability Assay.

FIG. 55 is a graph showing cell viability of Hep3B cells transfected with MLKL 1-180 mRNA constructs, as measured using the YOYO-3® cell viability read-out.

FIG. 56 is a graph showing ATP release from Hep3B cells transfected with MLKL 1-180 mRNA constructs, indicating necroptosis.

FIG. 57 is a graph showing HMGB1 release from HeLa cells transfected with MLKL 1-180 mRNA constructs, indicating necroptosis.

FIG. 58 is a graph showing cell surface staining of calreticulin on cells either mock transfected, transfected with an apoptosis-inducing construct (“PUMA”) or transfected with an MLKL construct, indicating necroptosis by the MLKL construct.

FIGS. 59A-59C are bar graphs showing cell viability of HeLa cells (FIG. 59A), B16F10 cells (FIG. 59B) or MC38 cells (FIG. 59C) transfected with MLKL, GSDMD or RIP3K mRNA constructs, as measured using the CellTiter-Glo® Luminescent Cell Viability Assay. *p<0.05; ***p<0.001 vs L2K ##p<0.01 vs HsMLKL (1-180).

FIG. 60 is a bar graph showing induction of death in NIH3T3 cells transfected with multimerizing RIPK3 mRNA constructs.

FIG. 61 is a bar graph showing induction of DAMP release (HMGB1 release) in B16F10 cells transfected with a multimerizing RIPK3 construct, indicating necroptosis.

FIG. 62 is a bar graph showing cell viability of SKOV3 cells transfected with DIABLO mRNA constructs, as measured using the CellTiter-Glo® Luminescent Cell Viability Assay.

FIG. 63 is a bar graph showing induction of cell death in HeLa cells transfected with caspase-4, caspase-5 or caspase-11 mRNA constructs. Results show mean±SEM from four independent experiments.

FIG. 64 is a bar graph showing induction of cell death in HeLa cells transfected with NLRP3, Pyrin or ASC mmRNA constructs. Results show mean±SEM from four independent experiments.

FIGS. 65A-65B are bar graphs showing activation of an interferon-sensitive response element (ISRE) by constitutively active IRF3 constructs (FIG. 65A) or IRF7 constructs (FIG. 65B).

FIG. 66 is a schematic illustration of the study design for the experimental results shown in FIG. 67.

FIG. 67 is a bar graph showing release of IL-18 by HeLa cells primed with an immune potentiator, as indicated, and transfected with a caspase-4, caspase-5 or caspase-11 construct, as indicated.

FIGS. 68A-68K are graphs showing the effect of treatment with the indicated executioner mRNA constructs, alone or in combination with the indicated immune checkpoint inhibitor, on growth of MC38 tumors in mice.

FIGS. 69A-69B are graphs showing the effect of treatment with the indicated executioner mRNA constructs, alone or in combination with the indicated immune potentiator and/or immune checkpoint inhibitor, on growth of MC38 tumors in mice (FIG. 69A) and on percent survival of mice (FIG. 69B).

FIGS. 70A-70B are graphs showing the effect of treatment with a STING mRNA construct in combination with anti-PD-1, as compared to vehicle alone or NT control +anti-PD-1, on growth of MC38 tumors in mice (FIG. 70A) and on percent survival of mice (FIG. 70B).

### DETAILED DESCRIPTION

The present disclosure provides compositions such as mRNAs constructs encoding a polypeptide that enhances immune responses to an antigen of interest, referred to herein as immune potentiator mRNA constructs or immune potentiator mRNAs, including chemically modified mRNAs (mmRNAs). The immune potentiator mRNAs of the disclosure enhance immune responses by, for example, activating Type I interferon pathway signaling, stimulating NFkB pathway signaling, or both, such that antigen-specific responses to an antigen of interest are stimulated. The immune potentiator mRNAs of the disclosure enhance immune responses to an endogenous antigen in a subject to which the immune potentiator mRNA is administered or enhance immune responses to an exogenous antigen that is administered to the subject with the immune potentiator mRNA (e.g., an mRNA construct encoding an antigen of interest that is coformulated and coadministered with the immune potentiator mRNA or an mRNA construct encoding an antigen of interest that is formulated and administered separately from the immune potentiator mRNA).

Surprisingly, it has been discovered that administration of an immune potentiator mRNA of the disclosure (e.g., an mRNA encoding a constitutively active STING polypeptide) or combination of immune potentiator mRNAs to a subject stimulates cytokine production (e.g., inflammatory cytokine production), stimulates antigen-specific CD8⁺ effector cell responses, stimulates antigen-specific CD4⁺ helper cell responses, increases the effector memory CD62L^{lo} T cell population and stimulates antigen-specific antibody production to an antigen of interest.

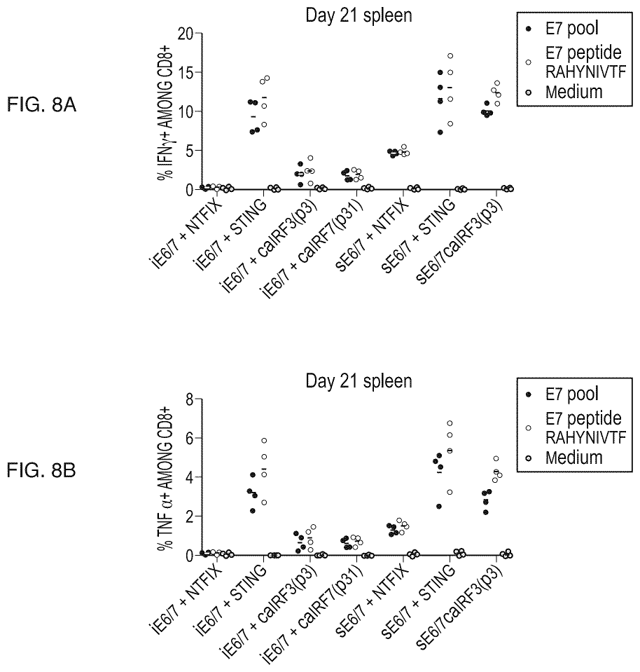

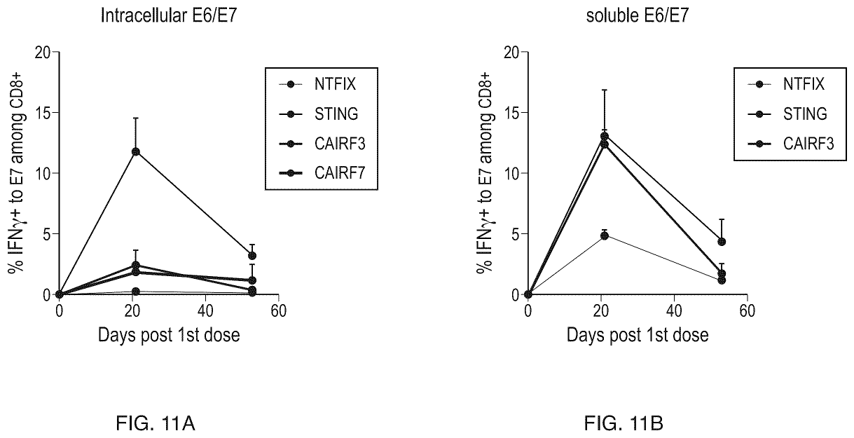

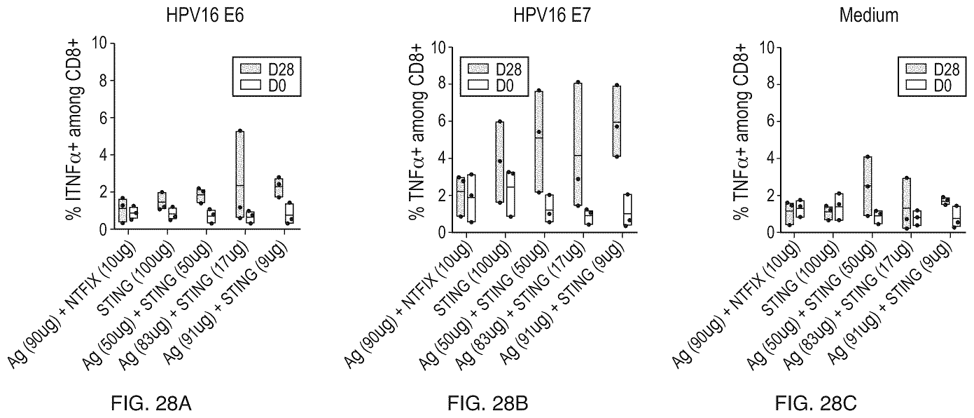

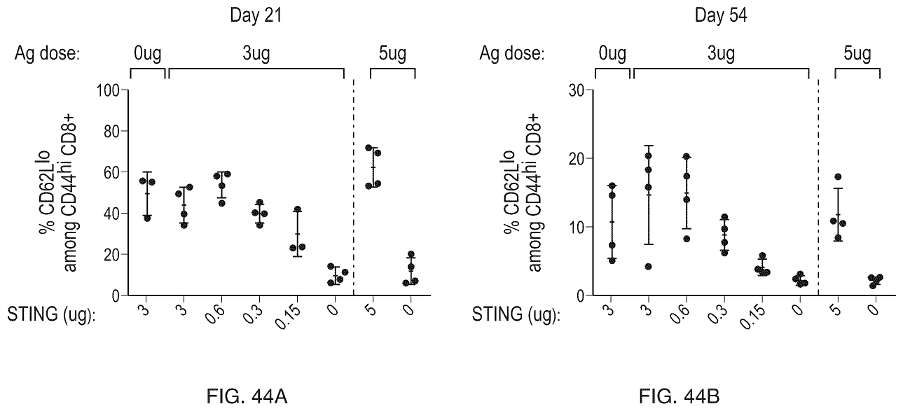

As described in detail in the examples, it has been found that administration of an immune potentiator mRNA construct (or combination of immune potentiator mRNAs) increases the percentage of CD8+ T cells that are positive by ICS for one or more cytokines (e.g., IFN-γ, TNFα and/or IL-2) in response to an antigen and increases the percentage of CD8+ T cells among the total T cell population (e.g., Example 5 and FIGS. 8-12). Remarkably, these effects were durable, as the higher percentage of antigen-specific CD8+ T cells positive by ICS for one or more cytokines was maintained for up to 7 weeks in vivo (FIG. 11). It was also found that administration of an immune potentiator mRNA construct (or combination of immune potentiator mRNAs) increases the effector memory CD62L^{lo} T cell population (e.g., Examples 5, 6, and Example 19), and that this effect is maintained over time (Example 19 and FIG. 44). Importantly, potentiation of antigen-specific T cell responses and antibody responses to an mRNA vaccine was also demonstrated in non-human primates (e.g., Example 12 and FIGS. 28-31).

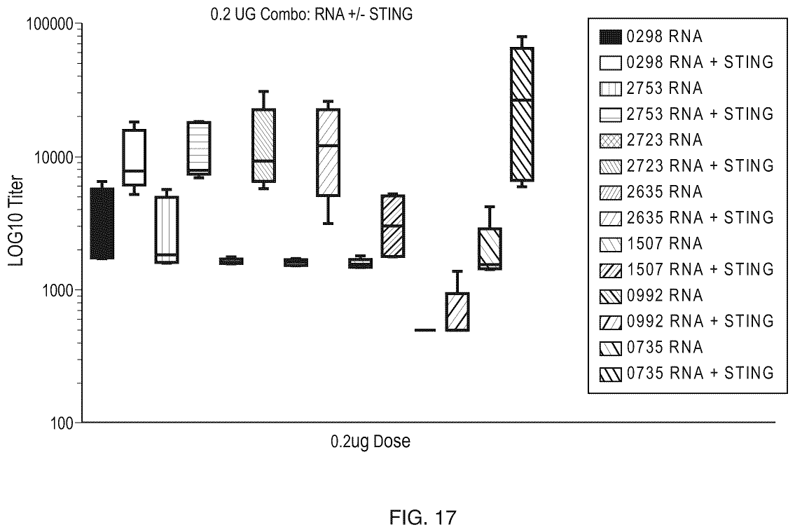

In the context of a bacterial vaccine, it has been shown that administration of an immune potentiator mRNA construct enhances humoral response to a bacterial vaccine by increasing antigen-specific antibody responses in vivo (e.g., Example 7 and FIG. 17).

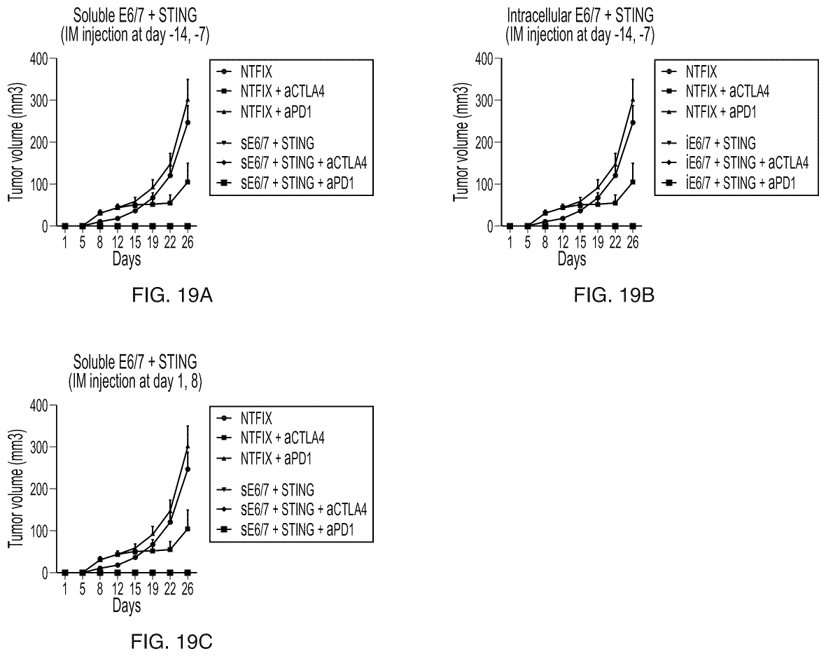

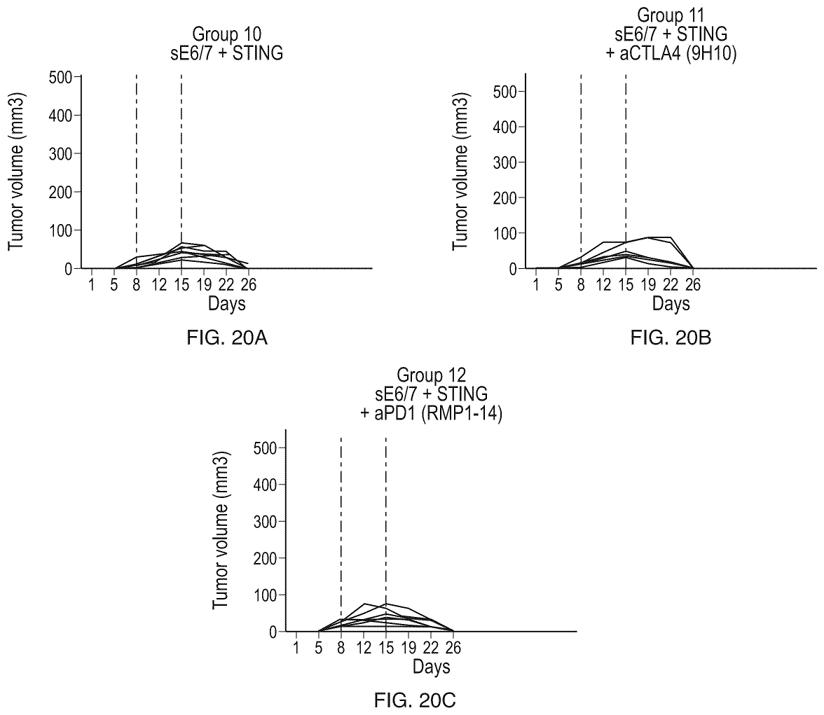

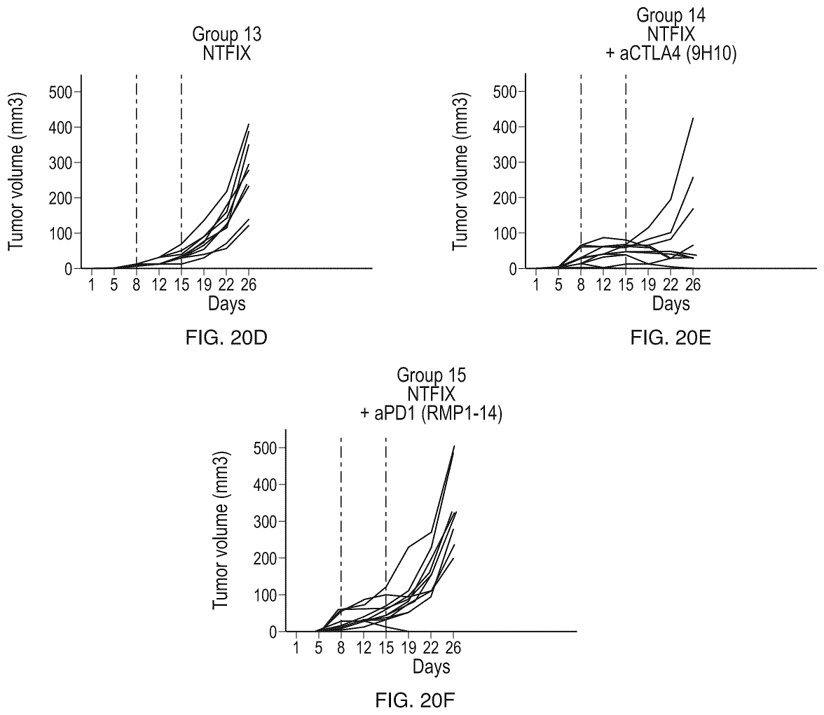

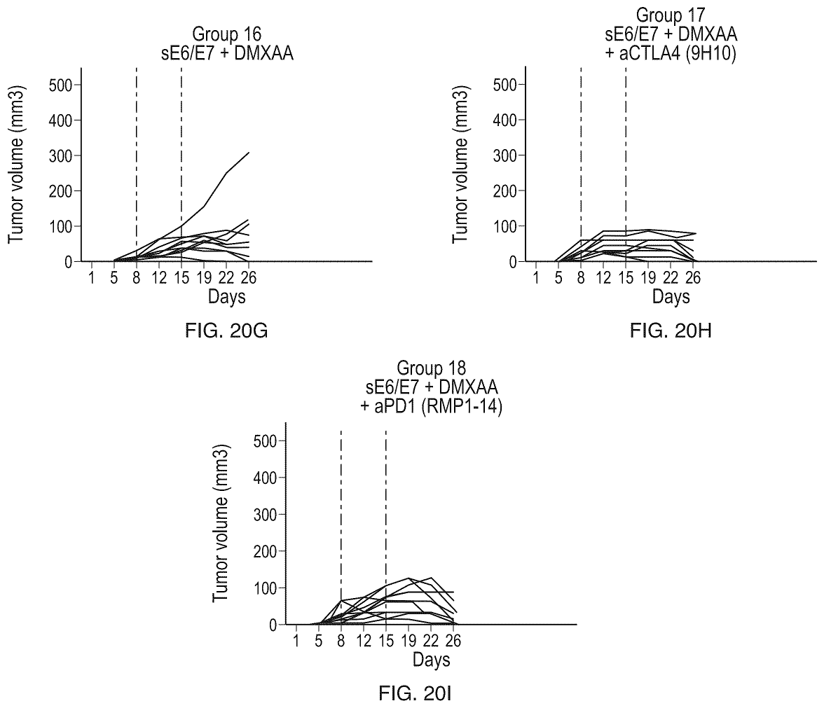

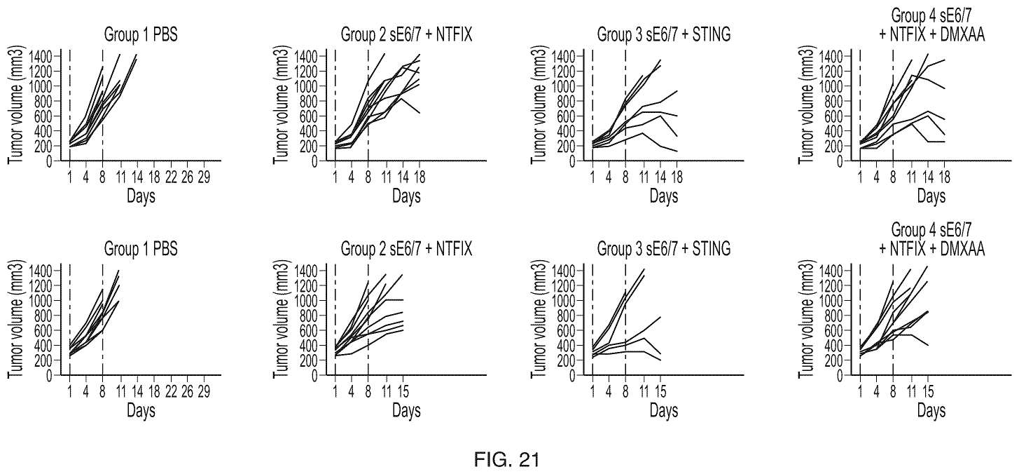

In the context of a cancer vaccine, administration of an immune potentiator mRNA construct was shown to result in a robust and durable immune response against cancer neoepitopes (Example 6) and was shown to potently inhibit tumor growth in prophylactic and therapeutic vaccination with an oncogenic viral vaccine (Example 10). For example, administration of an immune potentiator mRNA with an HPV vaccine was effective (alone or in combination with a checkpoint inhibitor) in preventing growth of HPV-expressing tumor cells in vivo (FIG. 19) and therapeutic vaccination (i.e., subsequent to tumor challenge) with the HPV vaccine together with the immune potentiator mRNA (alone or in combination with a checkpoint inhibitor) was effective in inducing regression of HPV-expressing tumors in vivo (FIG. 20). Notably, administration of an immune potentiator mRNA with the therapeutic vaccine also exhibited efficacy in inhibiting large, established tumors in vivo (FIG. 21).

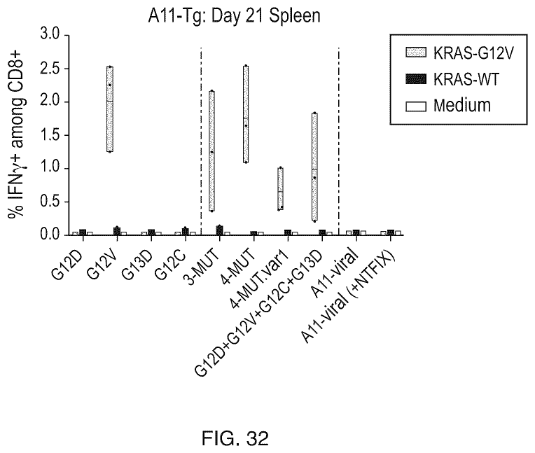

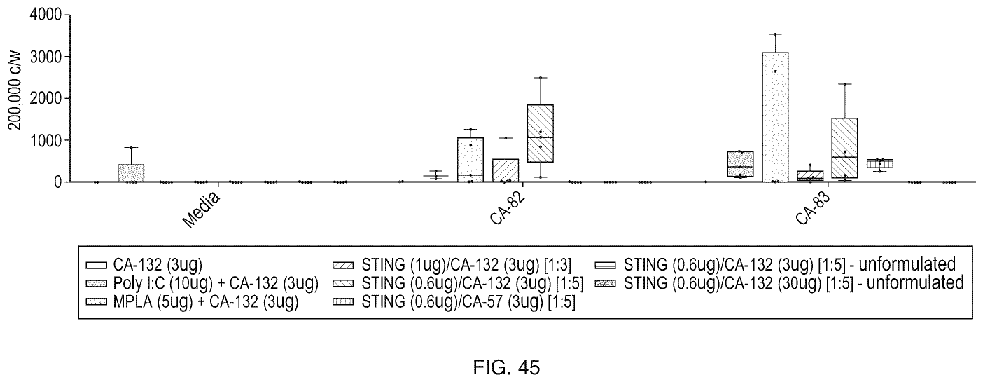

In the context of a personalized cancer vaccine, it has been shown that administration of an immune potentiator mRNA construct enhances antigen-specific T cell responses and antibody responses to an mRNA encoding a personalized cancer vaccine (a concatemer) inducing both Class I and Class II MCH responses (e.g., Example 20 and FIGS. 45-53). Administration of an immune potentiator mRNA was also found to potentiate immune responses to mRNA encoding KRAS cancer antigens in various formats (monomers and concatemer) (e.g., Example 13 and FIGS. 32-36).

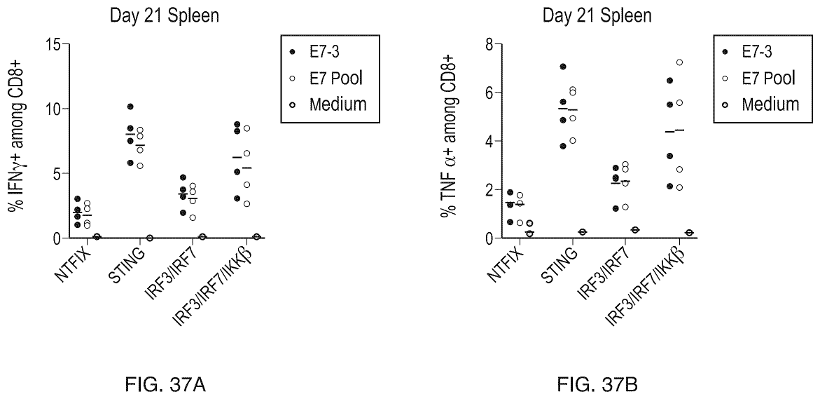

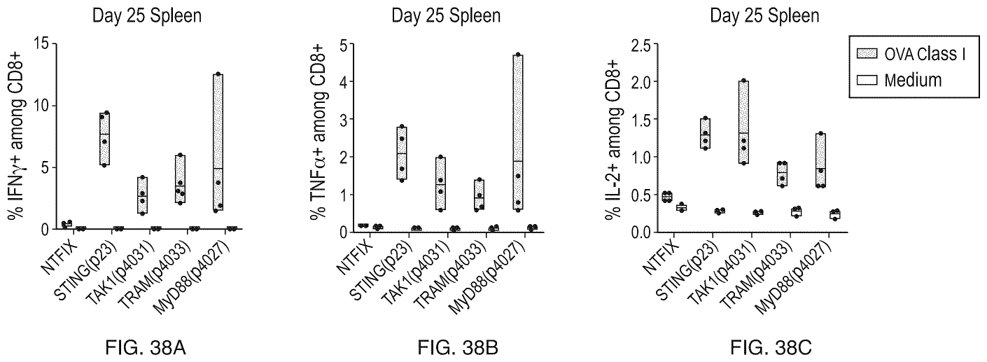

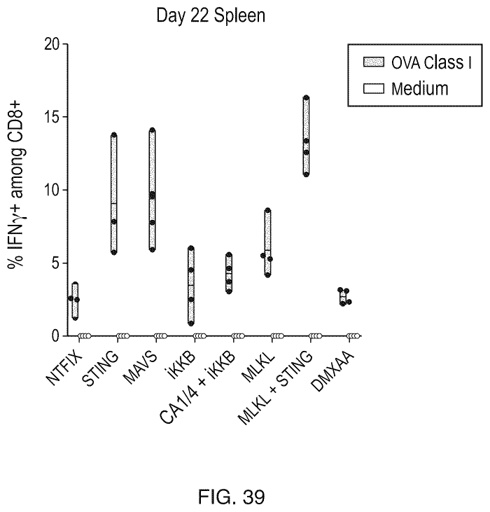

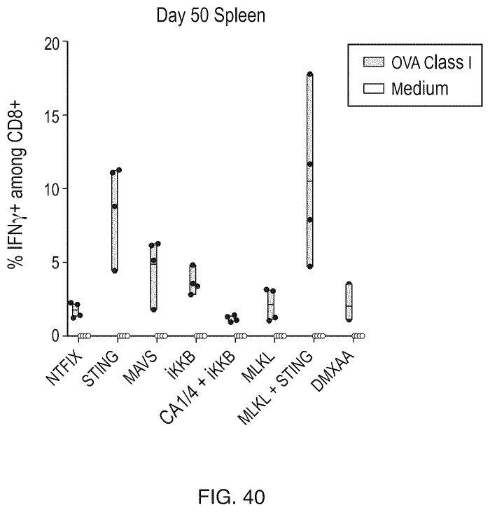

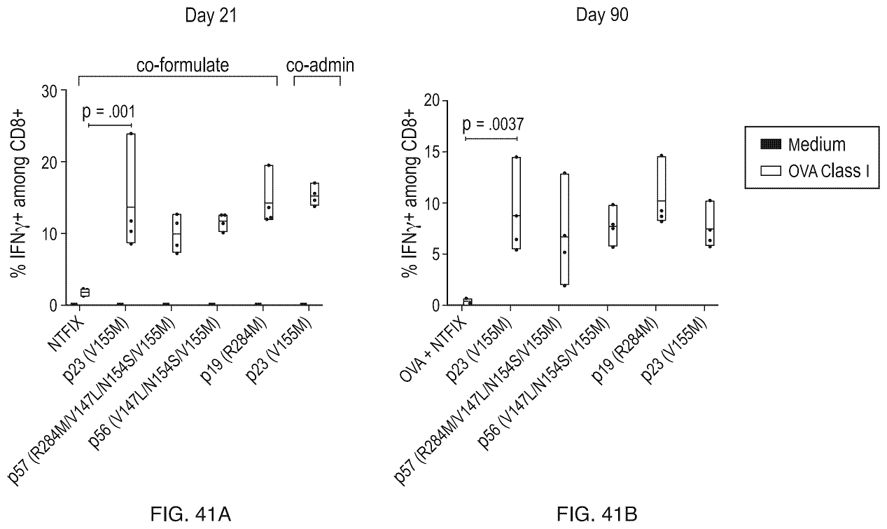

It has also been demonstrated that combinations of immune potentiator mRNAs encoding Type I interferon inducers and NFkB activators (e.g., Example 14 and FIG. 37), as well as immune potentiator mRNAs encoding components of intracellular signaling pathways that function downstream of TLRs (e.g., Example 15 and FIG. 38) potentiate antigen-specific T cell responses. Additional combinations of immune potentiator mRNAs encoding adaptor proteins (e.g., STING or MAVS), NFkB activators (e.g., IKKβ), inductors of inflammasome (e.g., caspases 1/4) and inductors of necroptosome (e.g., MLKL) were also shown to potentiate antigen-specific T cell responses. Surprisingly, the combination of an mRNA encoding an adaptor protein (e.g., STING) and an mRNA encoding an inducer of necroptosome (e.g., MLKL) exhibited enhanced activity as compared to an mRNA encoding MLKL alone (e.g., Example 16 and FIG. 39-40). The day 90 results demonstrate the immune potentiation effect was durable (e.g., Example 18 and FIG. 41).

Unexpectedly, it was found that the addition of an mRNA encoding an immune potentiator (e.g., STING) across a majority of antigen to immune potentiator (Ag:IP) ratios improved antigen-specific T cell responses compared to antigen alone (e.g., Example 20). The breadth of responsiveness was unexpected. For four of six antigens (epitopes) tested, the addition of an mRNA encoding an immune potentiator to antigen consistently produced higher T cell responses than antigen alone. Thus, it was discovered that there is a wide bell curve in the ratio of antigen to immune potentiator for improved immunogenicity.

It was also discovered that the addition of an mRNA encoding an immune potentiator (e.g., STING) across all antigens tested potentiates the immune response to the antigen relative to antigen alone. In most situations, at least a 2-fold increase in immune potentiation was found and, for certain antigens, an even greater enhancement of immune potentiation resulted (e.g., more than 5-fold, more than 10-fold, more than 20-fold, more than 30-fold, more than 50-fold, or more than 75-fold enhancement) (e.g., Example 21).

Accordingly, the present disclosure provides compositions comprising one or more mRNA constructs (e.g., one or more mmRNA constructs), wherein the one or more mRNA constructs encode an antigen(s) of interest and, in the same or a separate mRNA construct, encode a polypeptide that enhances an immune response to the antigen of interest. In some aspects, the disclosure provides nanoparticles, e.g., lipid nanoparticles, which include an immune potentiator mRNA that enhances an immune response, alone or in combination with mRNAs that encode an antigen of interest. The disclosure also provides pharmaceutical compositions comprising any of the mRNAs as described herein or nanoparticles, e.g., lipid nanoparticles comprising any of the mRNAs as described herein.

In another aspect, the disclosure provides compositions comprising one or more mRNA constructs (e.g., one or more mmRNA constructs) that encode a polypeptide that induces immunogenic cell death, such as necroptosis or pyroptosis. Such mRNA constructs can be used in combination with an immune potentiator mRNA construct of the disclosure to enhance the release of endogenous antigens in vivo to thereby stimulate an immune response against the endogenous antigens. In some aspects, the disclosure provides nanoparticles, e.g., lipid nanoparticles, which include an immunogenic cell death-inducing mRNA, alone or in combination with an immune potentiator mRNA. The disclosure also provides pharmaceutical compositions comprising any of the mRNAs as described herein or nanoparticles, e.g., lipid nanoparticles comprising any of the mRNAs as described herein.

In other aspects, the disclosure provides methods for enhancing an immune response to an antigen(s) of interest by administering to a subject an immune potentiator mRNA construct alone (for endogenous antigens) or by administering one or more mRNAs encoding an antigen(s) of interest and a mRNA encoding a polypeptide that enhances an immune response to the antigen(s) of interest, or lipid nanoparticle thereof, or pharmaceutical composition thereof, such that an immune response to the antigen of interest is enhanced in the subject. The methods of enhancing an immune response can be used, for example, to stimulate an immunogenic response to a tumor in a subject, to stimulate an immunogenic response to a pathogen in a subject or to enhance immune responses to a vaccine in a subject.

Immune Potentiator mRNAs

One aspect of the disclosure pertains to mRNAs that encode a polypeptide that stimulates or enhances an immune response against one or more antigens of interest. Such mRNAs that enhance immune responses to an antigen(s) of interest are referred to herein as immune potentiator mRNA constructs or immune potentiator mRNAs, including chemically modified mRNAs (mmRNAs). An immune potentiator of the disclosure enhances an immune response to an antigen of interest in a subject. The enhanced immune response can be a cellular response, a humoral response or both. As used herein, a “cellular” immune response is intended to encompass immune responses that involve or are mediated by T cells, whereas a “humoral” immune response is intended to encompass immune responses that involve or are mediated by B cells. An immune potentiator may enhance an immune response by, for example,
(i) stimulating Type I interferon pathway signaling;
(ii) stimulating NFkB pathway signaling;
(iii) stimulating an inflammatory response;
(iv) stimulating cytokine production; or
(v) stimulating dendritic cell development, activity or mobilization; and
(vi) a combination of any of (i)-(vi).

As used herein, “stimulating Type I interferon pathway signaling” is intended to encompass activating one or more components of the Type I interferon signaling pathway (e.g., modifying phosphorylation, dimerization or the like of such components to thereby activate the pathway), stimulating transcription from an interferon-sensitive response element (ISRE) and/or stimulating production or secretion of Type I interferon (e.g., IFN-α, IFN-β, IFN-∈, IFN-κ and/or IFN-ω). As used herein, “stimulating NFkB pathway signaling” is intended to encompass activating one or more components of the NFkB signaling pathway (e.g., modifying phosphorylation, dimerization or the like of such components to thereby activate the pathway), stimulating transcription from an NFkB site and/or stimulating production of a gene product whose expression is regulated by NFkB. As used herein, “stimulating an inflammatory response” is intended to encompass stimulating the production of inflammatory cytokines (including but not limited to Type I interferons, IL-6 and/or TNFα). As used herein, “stimulating dendritic cell development, activity or mobilization” is intended to encompass directly or indirectly stimulating dendritic cell maturation, proliferation and/or functional activity.

In certain embodiments, the immune potentiator mRNA construct enhances an immune response to an antigen of interest by a fold magnitude, e.g., relative to the immune response to the antigen in the absence of the immune potentiator, or relative to a small molecular agonist that enhances an immune response to the antigen. For example, in various embodiments, the immune potentiator mRNA construct enhances an immune response to an antigen of interest at least 2-fold, 3-fold, 4-fold, 5-fold, 7.5-fold, 10-fold, 20-fold, 30-fold, 40-fold, 50-fold, 75-fold, or greater, as compared to, for example, the immune response to the antigen in the absence of the immune potentiator mRNA construct or as compared to, for example, the immune response to the antigen in the presence of a small molecular agonist of an immune response to the antigen. In some embodiments, the immune potentiator mRNA construct enhance an immune response to an antigen of antigerest by 0.3-1000 fold, 1-750 fold, 5-500 fold, 7-250 fold, or 10-100 fold, as compared to, for example, the immune response to the antigen in the absence of the immune potentiator mRNA construct or as compared to, for example, the immune response to the antigen in the presence of a small molecular agonist of an immune response to the antigen. The fold magnitude enhancement of an immune potentiator construct can be measured using standard methods known in the art (e.g., as described in the Examples). For example, the level of antigen-specific T cells expressing inflammatory cytokines (e.g., IFN-γ and/or TNF-α) can be assessed by, e.g., intracellular staining (ICS) or by ELISpot analysis, as described in the Examples.

In some aspects, the disclosure provides an mRNA encoding a polypeptide that stimulates or enhances an immune response in a subject in need thereof (e.g., potentiates an immune response in the subject) by, for example, inducing adaptive immunity (e.g., by stimulating Type I interferon production), stimulating an inflammatory response, stimulating NFkB signaling and/or stimulating dendritic cell (DC) development, activity or mobilization in the subject. In some aspects, administration of an immune potentiator mRNA to a subject in need thereof enhances cellular immunity (e.g., T cell-mediated immunity), humoral immunity (e.g., B cell-mediated immunity) or both cellular and humoral immunity in the subject. In some aspects, administration of an immune potentiator mRNA stimulates cytokine production (e.g., inflammatory cytokine production), stimulates antigen-specific CD8⁺ effector cell responses, stimulates antigen-specific CD4⁺ helper cell responses, increases the effector memory CD62L^{lo} T cell population, stimulates B cell activity or stimulates antigen-specific antibody production, including combinations of the foregoing responses. In some aspects, administration of an immune potentiator mRNA stimulates cytokine production (e.g., inflammatory cytokine production) and stimulates antigen-specific CD8⁺ effector cell responses. In some aspects, administration of an immune potentiator mRNA stimulates cytokine production (e.g., inflammatory cytokine production), and stimulates antigen-specific CD4⁺ helper cell responses. In some aspects, administration of an immune potentiator mRNA stimulates cytokine production (e.g., inflammatory cytokine production), and increases the effector memory CD62L^{lo} T cell population. In some aspects, administration of an immune potentiator mRNA stimulates cytokine production (e.g., inflammatory cytokine production), and stimulates B cell activity or stimulates antigen-specific antibody production.

In one embodiment, an immune potentiator increases antigen-specific CD8⁺ effector cell responses (cellular immunity). For example, an immune potentiator can increase one or more indicators of antigen-specific CD8⁺ effector cell activity, including but not limited to CD8⁺ T cell proliferation and CD8⁺ T cell cytokine production. For example, in one embodiment, an immune potentiator increases production of IFN-γ, TNFα and/or IL-2 by antigen-specific CD8+ T cells. In various embodiments, an immune potentiator can increase CD8+ T cell cytokine production (e.g., IFN-γ, TNFα and/or IL-2 production) in response to an antigen (as compared to CD8+ T cell cytokine production in the absence of the immune potentiator) by at least 5% or at least 10% or at least 15% or at least 20% or at least 25% or at least 30% or at least 35% or at least 40% or at least 45% or at least 50%. For example, T cells obtained from a treated subject can be stimulated in vitro with the antigen of interest and CD8+ T cell cytokine production can be assessed in vitro. CD8+ T cell cytokine production can be determined by standard methods known in the art, including but not limited to measurement of secreted levels of cytokine production (e.g., by ELISA or other suitable method known in the art for determining the amount of a cytokine in supernatant) and/or determination of the percentage of CD8+ T cells that are positive for intracellular staining (ICS) for the cytokine. For example, intracellular staining (ICS) of CD8+ T cells for expression of IFN-γ, TNFα and/or IL-2 can be carried out by methods known in the art (see e.g., the Examples). In one embodiment, an immune potentiator increases the percentage of CD8+ T cells that are positive by ICS for one or more cytokines (e.g., IFN-γ, TNFα and/or IL-2) in response to an antigen (as compared to the percentage of CD8+ T cells that are positive by ICS for the cytokine(s) in the absence of the immune potentiator) by at least 5% or at least 10% or at least 15% or at least 20% or at least 25% or at least 30% or at least 35% or at least 40% or at least 45% or at least 50%.

In yet another embodiment, an immune potentiator increases the percentage of CD8+ T cells among the total T cell population (e.g., splenic T cells and/or PBMCs), as compared to the percentage of CD8+ T cells in the absence of the immune potentiator. For example, an immune potentiator can increase the percentage of CD8+ T cells among the total T cell population by at least 5% or at least 10% or at least 15% or at least 20% or at least 25% or at least 30% or at least 35% or at least 40% or at least 45% or at least 50%, as compared to the percentage of CD8+ T cells in the absence of the immune potentiator. The total percentage of CD8+ T cells among the total T cell population can be determined by standard methods known in the art, including but not limited to fluorescent activated cell sorting (FACS) or magnetic activated cell sorting (MACS).

In another embodiment, an immune potentiator increases a tumor-specific immune cell response, as determined by a decrease in tumor volume in vivo in the presence of the immune potentiator as compared to tumor volume in the absence of the immune potentiator. For example, an immune potentiator can decrease tumor volume by at least 5% or at least 10% or at least 15% or at least 20% or at least 25% or at least 30% or at least 35% or at least 40% or at least 45% or at least 50%, as compared to tumor volume in the absence of the immune potentiator. Measurement of tumor volume can be determined by methods well established in the art.

In another embodiment, an immune potentiator increases B cell activity (humoral immune response), for example by increasing the amount of antigen-specific antibody production, as compared to antigen-specific antibody production in the absence of the immune potentiator. For example, an immune potentiator can increase antigen-specific antibody production by at least 5% or at least 10% or at least 15% or at least 20% or at least 25% or at least 30% or at least 35% or at least 40% or at least 45% or at least 50%, as compared to antigen-specific antibody production in the absence of the immune potentiator. In one embodiment, antigen-specific IgG production is evaluated. Antigen-specific antibody production can be evaluated by methods well established in the art, including but not limited to ELISA, RIA and the like that measure the level of antigen-specific antibody (e.g., IgG) in a sample (e.g., a serum sample).

In another embodiment, an immune potentiator increases the effector memory CD62L^{lo} T cell population. For example, an immune potentiator can increase the total % of CD62L^{lo} T cells among CD8+ T cells. Among other functions, the effector memory CD62L10 T cell population has been shown to have an important function in lymphocyte trafficking (see e.g., Schenkel, J. M. and Masopust, D. (2014) Immunity 41:886-897). In various embodiments, an immune potentiator can increase the total percentage of effector memory CD62L^{lo} T cells among the CD8+ T cells in response to an antigen (as compared to the total percentage of CD62L^{lo} T cells among the CD8+ T cells population in the absence of the immune potentiator) by at least 5% or at least 10% or at least 15% or at least 20% or at least 25% or at least 30% or at least 35% or at least 40% or at least 45% or at least 50%. The total percentage of effector memory CD62L^{lo} T cells among the CD8+ T cells can be determined by standard methods known in the art, including but not limited to fluorescent activated cell sorting (FACS) or magnetic activated cell sorting (MACS).

The ability of an immune potentiator mRNA construct to enhance an immune response to an antigen of interest has been shown to be durable, with enhanced immunogenicity observed for extended periods of time, e.g., as long as 90 days. Accordingly, in various embodiments, an immune potentiator mRNA construct can enhance antigen-specific immune responses for at least 2 weeks, at least 3 weeks, at least 4 weeks, ate least one month, at least 5 weeks, at least 6 weeks, at least 7 weeks, at least 8 weeks, at least 9 weeks, at least 10 weeks, at least 11, weeks, at least 12 weeks, at least one month, at least 2 months or at least 3 months, or longer.

The ability of an immune potentiator mRNA construct to enhance an immune response to an antigen of interest can be evaluated in mouse model systems known in the art. In one embodiment, an immune competent mouse model system is used. In one embodiment, the mouse model system comprises C57/B16 mice (e.g., to evaluate antigen-specific CD8+ T cell responses to an antigen of interest, such as described in the Examples). In another embodiment, the mouse model system comprises BalbC mice or CD1 mice (e.g., to evaluate B cell responses, such an antigen-specific antibody responses).

In some embodiments, an immune potentiator polypeptide of the disclosure functions downstream of at least one Toll-like receptor (TLR) to thereby enhance an immune response. Accordingly, in one embodiment, the immune potentiator is not a TLR but is a molecule within a TLR signaling pathway downstream from the receptor itself. In some embodiments, the polypeptide stimulates a Type I interferon (IFN) response. Non-limiting examples of polypeptides that stimulate a Type I IFN response that are suitable for use as an immune potentiator include STING, MAVS, IRF1, IRF3, IRF5, IRF7, IRF8, IRF9, TBK1, IKKα, IKKi, MyD88, TRAM, TRAF3, TRAF6, IRAK1, IRAK4, TRIF, IPS-1, RIG-1, DAI and IFI16. Specific examples of polypeptides that stimulate a Type I interferon (IFN) response are described further below.

In another embodiment, the polypeptide stimulates an NFkB-mediated proinflammatory response. Non-limiting examples of polypeptides that stimulate an NFkB-mediated proinflammatory response include STING, c-FLIP, IKKβ, RIPK1, Btk, TAK1, TAK-TAB1, TBK1, MyD88, IRAK1, IRAK2, IRAK4, TAB2, TAB3, TRAF6, TRAM, MKK3, MKK4, MKK6 and MKK7. Specific examples of polypeptides that stimulate an NFκB-mediated proinflammatory response are described further below.

In another embodiment, the polypeptide is an intracellular adaptor protein. In one embodiment, the intracellular adaptor protein stimulates a Type I IFN response. In another embodiment, the intracellular adaptor protein stimulates an NFkB-mediated proinflammatory response. Non-limiting examples of intacellular adaptor proteins include STING, MAVS and MyD88. Specific examples of intracellular adaptor proteins are described further below.

In another embodiment, the polypeptide is an intracellular signaling protein. In one embodiment, the polypeptide is an intracellular signaling protein of a TLR signaling pathway. In one embodiment, the intracellular signalling protein stimulates a Type I IFN response. In another embodiment, the intracellular signalling protein stimulates an NFkB-mediated proinflammatory response. Non-limiting examples of intacellular signalling proteins include MyD88, IRAK 1, IRAK2, IRAK4, TRAF3, TRAF6, TAK1, TAB2, TAB3, TAK-TAB1, MKK3, MKK4, MKK6, MKK7, IKKα, IKKβ, TRAM, TRIF, RIPK1, and TBK1. Specific examples of intracellular signaling proteins are described further below.

In another embodiment, the polypeptide is a transcription factor. In one embodiment, the transcription factor stimulates a Type I IFN response. In another embodiment, the transcription factor stimulates an NFkB-mediated proinflammatory response. Non-limiting examples of transcription factors include IRF3 or IRF7. Specific examples of transcription factors are described further below.

In another embodiment, the polypeptide is involved in necroptosis or necroptosome formation. A polypeptide is “involved in” necroptosis or necroptosome formation if the protein mediates necroptosis itself or participates with additional molecules in mediating necroptosis and/or in necroptosome formation. Non-limiting examples of polypeptides involved in necroptosis or necroptosome formation include MLKL, RIPK1, RIPK3, DIABLO and FADD. Specific examples of polypeptides involved in necroptosis or necroptosome formation are described further below.

In another embodiment, the polypeptide is involved in pyroptosis or inflammasome formation. A polypeptide is “involved in” pyroptosis or inflammasome formation if the protein mediates pyroptosis itself or participates with additional molecules in mediating pyroptosis and/or in inflammasome formation. Non-limiting examples of polypeptides involved in pyroptosis or inflammasome formation include caspase 1, caspase 4, caspase 5, caspase 11, GSDMD, NLRP3, Pyrin domain and ASC/PYCARD. Specific examples of polypeptides involved in pyroptosis or inflammasome formation are described further below.

In some embodiments, an mRNA of the disclosure encoding an immune potentiator can comprises one or more modified nucleobases. Suitable modifications are discussed further below.

In some embodiments, an mRNA of the disclosure encoding an immune potentiator is formulated into a lipid nanoparticle. In one embodiment, the lipid nanoparticle further comprises an mRNA encoding an antigen of interest. In one embodiment, the lipid nanoparticle is administered to a subject to enhance an immune response against the antigen of interest in the subject. Suitable nanoparticles and methods of use are discussed further below.

In another embodiment, the disclosure provides compositions that comprise combinations of two or more immune potentiator mRNAs. The two or more immune potentiator mRNAs can be immune potentiators of the same type (e.g., two or more immune potentiators that stimulate a Type I interferon (IFN) response) or can be immune potentiators of different types. Accordingly, in one embodiment, the disclosure provides a composition comprising a first messenger RNA (mRNA) encoding a first polypeptide that enhances an immune response to an antigen of interest in a subject, a second mRNA encoding a second polypeptide that enhances an immune response to an antigen of interest in a subject and, optionally, a third mRNA encoding a third polypeptide that enhances an immune response to an antigen of interest in a subject (and optionally, fourth, fifth, sixth or more mRNAs encoding immune potentiators), wherein the immune response comprises a cellular or humoral immune response characterized by:

(i) stimulating Type I interferon pathway signaling;
(ii) stimulating NFkB pathway signaling;
(iii) stimulating an inflammatory response;
(iv) stimulating cytokine production; or
(v) stimulating dendritic cell development, activity or mobilization; and
(vi) a combination of any of (i)-(vi).

In some embodiments, the first, second and/or, optionally, third polypeptides (and optionally, fourth, fifth, sixth or more polypeptides) function downstream of at least one Toll-like receptor (TLR) to thereby enhance an immune response.

In various embodiments of the combination compositions:
(i) the first polypeptide stimulates a Type I interferon (IFN) response and the second polypeptide stimulates an NFkB-mediated proinflammatory response;
(ii) the first polypeptide stimulates a Type I interferon (IFN) response and the second polypeptide is involved in necroptosis or necroptosome formation;
(iii) the first polypeptide stimulates a Type I interferon (IFN) response and the second polypeptide is involved in pyroptosis or inflammasome formation;
(iv) the first polypeptide stimulates an NFkB-mediated proinflammatory response and the second polypeptide is involved in necroptosis or necroptosome formation;
(v) the first polypeptide stimulates an NFkB-mediated proinflammatory response and the second polypeptide is involved in pyroptosis or inflammasome formation;
(vii) the first polypeptide stimulates a Type I interferon (IFN) response, the second polypeptide stimulates an NFkB-mediated proinflammatory response and the third polypeptide is involved in necroptosis or necroptosome formation; or
(viii) the first polypeptide stimulates a Type I interferon (IFN) response, the second polypeptide stimulates an NFkB-mediated proinflammatory response and the third polypeptide is involved in pyroptosis or inflammasome formation.

Suitable non-limiting examples of each of these categories of immune potentiators are listed above and described in further detail below. All combinations of the listed immune potentiators are contemplated.

In some embodiments, the first polypeptide stimulates a Type I interferon (IFN) response and is selected from the group consisting of STING, MAVS, IRF1, IRF3, IRF5, IRF7, IRF8, IRF9, TBK1, IKKα, IKKi, MyD88, TRAM, TRAF3, TRAF6, IRAK1, IRAK4, TRIF, IPS-1, RIG-1, DAI and IFI16; and the second polypeptide stimulates an NFkB-mediated proinflammatory response and is selected from the group consisting of STING, c-FLIP, IKKβ, RIPK1, Btk, TAK1, TAK-TAB1, TBK1, MyD88, IRAK1, IRAK2, IRAK4, TAB2, TAB3, TRAF6, TRAM, MKK3, MKK4, MKK6 and MKK7. In some embodiments, the first polypeptide is a constitutively active IRF3 and the second polypeptide is a constitutively active IKKβ. In some embodiments, the composition further comprises an mRNA encoding a constitutively active IRF7 polypeptide (i.e., the composition comprises mRNAs encoding constitutively active IRF3, constitutively active IRF7 polypeptide and constitutively active IKKβ).

In some embodiments, the first polypeptide stimulates a Type I interferon (IFN) response and is selected from the group consisting of STING, MAVS, IRF1, IRF3, IRF5, IRF7, IRF8, IRF9, TBK1, IKKα, IKKi, MyD88, TRAM, TRAF3, TRAF6, IRAK1, IRAK4, TRIF, IPS-1, RIG-1, DAI and IFI16; and the second polypeptide is involved in necroptosis or necroptosome formation and is selected from the group consisting of MLKL, RIPK1, RIPK3, DIABLO and FADD. In some embodiments, the first polypeptide is a constitutively active STING and the second polypeptide is an MLKL polypeptide.

In some embodiments, the first polypeptide stimulates an NFkB-mediated proinflammatory response and is selected from the group consisting of STING, c-FLIP, IKKβ, RIPK1, Btk, TAK1, TAK-TAB1, TBK1, MyD88, IRAK1, IRAK2, IRAK4, TAB2, TAB3, TRAF6, TRAM, MKK3, MKK4, MKK6 and MKK7; and the second polypeptide is involved in pyroptosis or inflammasome formation and is selected from the group consisting of caspase 1, caspase 4, caspase 5, caspase 11, GSDMD, NLRP3, Pyrin domain and ASC/PYCARD. In some embodiments, the first polypeptide is a constitutively active IKKβ and the second polypeptide is a caspase-1 polypeptide. In some embodiments, the composition further comprises an mRNA encoding a caspase-4 polypeptide (i.e., the composition comprises mRNAs encoding a constitutively active IKKβ, a caspase-1 polypeptide and a caspase-4 polypeptide).

In some embodiments, a combination composition of the disclosure encoding two or more immune potentiators comprises one or more mRNAs that comprises one or more modified nucleobases. Suitable modifications are discussed further below.

In some embodiments, a combination composition of the disclosure encoding two or more immune potentiators is formulatined into a lipid nanoparticle. In some embodiments, the lipid nanoparticle further comprises an mRNA encoding an antigen of interest. In some embodiments, the lipid nanoparticle is administered to a subject to enhance an immune response against the antigen of interest in the subject. Suitable nanoparticles and methods of use are discussed further below.

Immune Potentiators mRNAs that Stimulate Type I Interferon

In some aspects, the disclosure provides an immune potentiator mRNA encoding a polypeptide that stimulates or enhances an immune response against an antigen of interest by simulating or enhancing Type I interferon pathway signaling, thereby stimulating or enhancing Type I interferon (IFN) production. It has been established that successful induction of anti-tumor or anti-microbial adaptive immunity requires Type I IFN signaling (see e.g., Fuertes, M. B. et al. (2013) Trends Immunol. 34:67-73). The production of Type I IFNs (including IFN-α, IFN-β, IFN-∈, IFN-κ and IFN-ω) plays a role in clearance of microbial infections, such as viral infections. It has also been appreciated that host cell DNA (for example derived from damaged or dying cells) is capable of inducing Type I interferon production and that the Type I IFN signaling pathway plays a role in the development of adaptive anti-tumor immunity. However, many pathogens and cancer cells have evolved mechanisms to reduce or inhibit Type I interferon responses. Thus, activation (including stimulation and/or enhancement) of the Type I IFN signaling pathway in a subject in need thereof, by providing an immune potentiator mRNA of the disclosure to the subject, stimulates or enhances an immune response in the subject in a wide variety of clinical situations, including treatment of cancer and pathogenic infections, as well as in potentiating vaccine responses to provide protective immunity.

Type I interferons (IFNs) are pro-inflammatory cytokines that are rapidly produced in multiple different cell types, typically upon viral infection, and known to have a wide variety of effects. The canonical consequences of type I IFN production in vivo is the activation of antimicrobial cellular programs and the development of innate and adaptive immune responses. Type I IFN induces a cell-intrinsic antimicrobial state in infected and neighboring cells that limits the spread of infectious agents, particularly viral pathogens. Type I IFN also modulates innate immune cell activation (e.g., maturation of dendritic cells) to promote antigen presentation and nature killer cell functions. Type I IFN also promotes the development of high-affinity antigen-specific T and B cell responses and immunological memory (Ivashkiv and Donlin (2014) Nat Rev Immunol 14(1):36-49)

Type I IFN activates dendritic cells (DCs) and promotes their T cell stimulatory capacity through autocrine signaling (Montoya et al., (2002) Blood 99:3263-3271). Type I IFN exposure facilitates maturation of DCs via increasing the expression of chemokine receptors and adhesion molecules (e.g., to promote DC migration into draining lymph nodes), co-stimulatory molecules, and MHC class I and class II antigen presentation. DCs that mature following type I IFN exposure can effectively prime protective T cell responses (Wijesundara et al., (2014) Front Immunol 29 (412) and references therein).

Type I IFN can either promote or inhibit T cell activation, proliferation, differentiation and survival depending largely on the timing of type I IFN signaling relative to T cell receptor signaling (Crouse et al., (2015) Nat Rev Immunol 15:231-242). Early studies revealed that MHC-I expression is upregulated in response to type I IFN in multiple cell types (Lindahl et al., (1976), J Infect Dis 133 (Suppl): A66-A68; Lindahl et al., (1976) Proc Natl Acad Sci USA 17:1284-1287) which is a requirement for optimal T cell stimulation, differentiation, expansion and cytolytic activity. Type I IFN can exert potent co-stimulatory effects on CD8 T cells, enhancing CD8 T cell proliferation and differentiation (Curtsinger et al., (2005) J Immunol 174:4465-4469; Kolumam et al., (2005) J Exp Med 202:637-650).

Similar to effects on T cells, type I IFN signaling has both positive and negative effects on B cell responses depending on the timing and context of exposure (Braun et al., (2002) Int Immunol 14(4):411-419; Lin et al, (1998) 187(1):79-87). The survival and maturation of immature B cells can be inhibited by type I IFN signaling. In contrast to immature B cells, type I IFN exposure has been shown to promote B cell activation, antibody production and isotype switch following viral infection or following experimental immunization (Le Bon et al., (2006) J Immunol 176:4:2074-2078; Swanson et al., (2010) J Exp Med 207:1485-1500).

A number of components involved in Type I IFN pathway signaling have been established, including STING, Interferon Regulatory Factors, such as IRF1, IRF3, IRF5, IRF7, IRF8, and IRF9, TBK1, IKKi, MyD88, MAVS and TRAM. Additional components involved in Type I IFN pathway signaling include IKKα, TRAF3, TRAF6, IRAK-1, IRAK-4, TRIF, IPS-1, TLR-3, TLR-4, TLR-7, TLR-8, TLR-9, RIG-1, DAI and IFI16.

Accordingly, in one embodiment, an immune potentiator mRNA encodes any of the foregoing components involved in Type I IFN pathway signaling.

Immune Potentiator mRNA Encoding STING

The present disclosure encompasses mRNA (including mmRNA) encoding STING, including constitutively active forms of STING, as immune potentiators. STING (STimulator of INterferon Genes; also known as transmembrane protein 173 (TMEM173), mediator of IRF3 activation (MITA), methionine-proline-tyrosine-serine (MPYS), and ER IFN stimulator (ERIS)) is a 379 amino acid, endoplasmic reticulum (ER) resident transmembrane protein that functions as a signaling molecule controlling the transcription of immune response genes, including type I IFNs and pro-inflammatory cytokines (Ishikawa & Barber, (2008) Nature 455:647-678; Ishikawa et al., (2009) Nature 461:788-792; Barber (2010) Nat Rev Immunol 15(12):760-770).

STING functions as a signaling adaptor linking the cytosolic detection of DNA to the TBK1/IRF3/Type I IFN signaling axis. The signaling adaptor functions of STING are activated through the direct sensing of cyclic dinucleotides (CDNs). Examples of CDNs include cyclic di-GMP (guanosine 5′-monophosphate), cyclic di-AMP (adenosine 5′-monophosphate) and cyclic GMP-AMP (cGAMP). Initially characterized as ubiquitous bacterial secondary messengers, CDNs are now known to constitute a class of pathogen-associated molecular pattern molecules (PAMPs) that activate the TBK1/IRF3/type I IFN signaling axis via direct interaction with STING. STING is capable of sensing aberrant DNA species and/or CDNs in the cytosol of the cell, including CDNs derived from bacteria, and/or from the host protein cyclic GMP-AMP synthase (cGAS). The cGAS protein is a DNA sensor that produces cGAMP in response to detection of DNA in the cytosol (Burdette et al., (2011) Nature 478:515-518; Sun et al., (2013) Science 339:786-791; Diner et al., (2013) Cell Rep 3:1355-1361; Ablasser et al., (2013) Nature 498:380-384).

Upon binding to a CDN, STING dimerizes and undergoes a conformational change that promotes formation of a complex with TANK-binding kinase 1 (TBK1) (Ouyang et al., (2012) Immunity 36(6):1073-1086). This complex translocates to the perinuclear Golgi, resulting in delivery of TBK1 to endolysosomal compartments where it phosphorylates IRF3 and NF-κB transcription factors (Zhong et al., (2008) Immunity 29:538-550). A recent study has shown that STING functions as a scaffold by binding to both TBK1 and IRF3 to specifically promote the phosphorylation of IRF3 by TBK1 (Tanaka & Chen, (2012) Sci Signal 5(214):ra20). Activation of the IRF3-, IRF7- and NF-κB-dependent signaling pathways induces the production of cytokines and other immune response-related proteins, such as type I IFNs, which promote anti-pathogen and/or anti-tumor activity.

A number of studies have investigated the use of CDN agonists of STING as potential vaccine adjuvants or immunomodulatory agents to elicit humoral and cellular immune responses (Dubensky et al., (2013) Ther Adv Vaccines 1(4):131-143 and references therein). Initial studies demonstrated that administration of the CDN c-di-GMP attenuated Staphylococcus aureus infection in vivo, reducing the number of recovered bacterial cells in a mouse infection model yet c-di-GMP had no observable inhibitory or bactericidal effect on bacterial cells in vitro suggesting the reduction in bacterial cells was due to an effect on the host immune system (Karaolis et al., (2005) Antimicrob Agents Chemother 49:1029-1038; Karaolis et al., (2007) Infect Immun 75:4942-4950). Recent studies have shown that synthetic CDN derivative molecules formulated with granulocyte-macrophage colony-stimulating factor (GM-CSF)-producing cancer vaccines (termed STINGVAX) elicit enhanced in vivo antitumor effects in therapeutic animal models of cancer as compared to immunization with GM-CSF vaccine alone (Fu et al., (2015) Sci Transl Med 7(283) 283ra52), suggesting that CDN are potent vaccine adjuvants.

Mutant STING proteins resulting from polymorphisms mapped to the human TMEM173 gene have been described exhibiting a gain-of function or constitutively active phenotype. When expressed in vitro, mutant STING alleles were shown to potently stimulate induction of type I IFN (Liu et al., (2014) N Engl J Med 371:507-518; Jeremiah et al., (2014) J Clin Invest 124:5516-5520; Dobbs et al., (2015) Cell Host Microbe 18 (2): 157-168; Tang & Wang, (2015) PLOS ONE 10 (3): e0120090; Melki et al., (2017) J Allergy Clin Immunol In Press; Konig et al., (2017) Ann Rheum Dis 76(2):468-472; Burdette et al. (2011) Nature 478:515-518).

Provided herein are mRNAs (including chemically modified mRNAs (mmRNAs)) encoding constitutively active forms of STING, including mutant human STING isoforms for use as immune potentiators as described herein. mRNAs encoding constitutively active forms of STING (e.g., mmRNAs), including mutant human STING isoforms are set forth in the Sequence Listing herein. The amino acid residue numbering for mutant human STING polypeptides used herein corresponds to that used for the 379 amino acid residue wild type human STING (isoform 1) available in the art as Genbank Accession Number NP_938023.

Accordingly, in one aspect, the disclosure provides a mRNA (e.g., mmRNA) encoding a mutant human STING protein having a mutation at amino acid residue 155, in particular an amino acid substitution, such as a V155M mutation. In one embodiment, the mRNA (e.g., mmRNA) encodes an amino acid sequence as set forth in SEQ ID NO:1. In one embodiment, the STING V155M mutant is encoded by a nucleotide sequence shown in SEQ ID NO: 199, 1319 or 1320. In one embodiment, the mRNA (e.g., mmRNA) comprises a 3′ UTR sequence as shown in SEQ ID NO: 209, which includes an miR122 binding site.

In other aspects, the disclosure provides a mRNA encoding a mutant human STING protein having a mutation at amino acid residue 284, such as an amino acid substitution. Non-limiting examples of residue 284 substitutions include R284T, R284M and R284K. In certain embodiments, the mutant human STING protein has as a R284T mutation, for example has the amino acid sequence set forth in SEQ ID NO: 2 or is encoded by an the nucleotide sequence shown in SEQ ID NO 200 or SEQ ID NO: 1442. In certain embodiments, the mutant human STING protein has a R284M mutation, for example has the amino acid sequence as set forth in SEQ ID NO: 3 or is encoded by the nucleotide sequence shown in SEQ ID NO: 201 or SEQ ID NO: 1443. In certain embodiments, the mutant human STING protein has a R284K mutation, for example has the amino acid sequence as set forth in SEQ ID NO: 4 or 224, or is encoded by the nucleotide sequence shown in SEQ ID NO: 202, 225, 1444 or 1466.

In other aspects, the disclosure provides a mRNA encoding a mutant human STING protein having a mutation at amino acid residue 154, such as an amino acid substitution, such as a N154S mutation. In certain embodiments, the mutant human STING protein has a N154S mutation, for example has the amino acid sequence as set forth in SEQ ID NO: 5 or is encoded by the nucleotide sequence shown in SEQ ID NO: 203 or SEQ ID NO: 1445.

In yet other aspects, the disclosure provides a mRNA encoding a mutant human STING protein having a mutation at amino acid residue 147, such as an amino acid substitution, such as a V147L mutation. In certain embodiments, the mutant human STING protein having a V147L mutation has the amino acid sequence as set forth in SEQ ID NO: 6 or is encoded by the nucleotide sequence shown in SEQ ID NO: 204 or SEQ ID NO: 1446.

In other aspects, the disclosure provides a mRNA encoding a mutant human STING protein having a mutation at amino acid residue 315, such as an amino acid substitution, such as a E315Q mutation. In certain embodiments, the mutant human STING protein having a E315Q mutation has the amino acid sequence as set forth in SEQ ID NO: 7 or is encoded by the nucleotide sequence shown in SEQ ID NO: 205 or SEQ ID NO: 1447.

In other aspects, the disclosure provides a mRNA encoding a mutant human STING protein having a mutation at amino acid residue 375, such as an amino acid substitution, such as a R375A mutation. In certain embodiments, the mutant human STING protein having a R375A mutation has the amino acid sequence as set forth in SEQ ID NO: 8 or is encoded by the nucleotide sequence shown in SEQ ID NO: 206 or SEQ ID NO: 1448.

In other aspects, the disclosure provides a mRNA encoding a mutant human STING protein having a one or more or a combination of two, three, four or more of the foregoing mutations. Accordingly, in one aspect the disclosure provides a mRNA encoding a mutant human STING protein having one or more mutations selected from the group consisting of: V147L, N154S, V155M, R284T, R284M, R284K, E315Q and R375A, and combinations thereof. In other aspects, the disclosure provides a mRNA encoding a mutant human STING protein having a combination of mutations selected from the group consisting of: V155M and R284T; V155M and R284M; V155M and R284K; V155M and V147L; V155M and N154S; V155M and E315Q; and V155M and R375A.

In other aspects, the disclosure provides a mRNA encoding a mutant human STING protein having a V155M and one, two, three or more of the following mutations: R284T; R284M; R284K; V147L; N154S; E315Q; and R375A. In other aspects, the disclosure provides a mRNA encoding a mutant human STING protein having V155M, V147L and N154S mutations. In other aspects, the disclosure provides a mRNA encoding a mutant human STING protein having V155M, V147L, N154S mutations, and, optionally, a mutation at amino acid 284. In yet other aspects, the disclosure provides a mRNA encoding a mutant human STING protein having V155M, V147L, N154S mutations, and a mutation at amino acid 284 selected from R284T, R284M and R284K. In other aspects, the disclosure provides a mRNA encoding a mutant human STING protein having V155M, V147L, N154S, and R284T mutations. In other aspects, the disclosure provides a mRNA encoding a mutant human STING protein having V155M, V147L, N154S, and R284M mutations. In other aspects, the disclosure provides a mRNA encoding a mutant human STING protein having V155M, V147L, N154S, and R284K mutations.

In other embodiments, the disclosure provides a mRNA encoding a mutant human STING protein having a combination of mutations at amino acid residue 147, 154, 155 and, optionally, 284, in particular amino acid substitutions, such as a V147L, N154S, V155M and, optionally, R284M. In certain embodiments, the mutant human STING protein has V147N, N154S and V155M mutations, such as the amino acid sequence as set forth in SEQ ID NO: 9 or encoded by the nucleotide sequence shown in SEQ ID NO: 207 or SEQ ID NO: 1449. In certain embodiments, the mutant human STING protein has R284M, V147N, N154S and V155M mutations, such as the amino acid sequence as set forth in SEQ ID NO: 10 or encoded by the nucleotide sequence shown in SEQ ID NO: 208 or SEQ ID NO: 1450.

In another embodiment, the disclosure provides a mRNA encoding a mutant human STING protein that is a constitutively active truncated form of the full-length 379 amino acid wild type protein, such as a constitutively active human STING polypeptide consisting of amino acids 137-379.

Immune Potentiator mRNA Encoding Immune Regulatory Factor (IRF)

The present disclosure provides mRNA (including mmRNA) encoding Interferon Regulatory Factors, such as IRF1, IRF3, IRF5, IRF7, IRF8, and IRF9 as immune potentiators. The IRF transcription factor family is involved in the regulation of gene expression leading to the production of type I interferons (IFNs) during innate immune responses. Nine human IRFs have been identified to date (IRF-1-IRF-9), with each family member sharing extensive sequence homology within their N-terminal binding domains (DBDs) (Mamane et al., (1999) Gene 237:1-14; Taniguchi et al., (2001) Annu Rev Immunol 19:623-655). Within the IRF family, IRF1, IRF3, IRF5, and IRF7 have been specifically implicated as positive regulators of type I IFN gene transcription (Honda et al., (2006) Immunity 25(3):349-360). IRF1 was the first family member discovered to activate type I IFN gene promoters (Miyamoto et al., (1988) Cell 54:903-913). Although studies show that IRF1 participates in type I IFN gene expression, normal induction of type I IFN was observed in virus-infected IRF1−/− murine fibroblasts, suggesting dispensability (Matsuyama et al., (1993) Cell 75:83-97). IRF5 was also shown to be dispensable for type I IFN induction by viruses or TLR agonists (Takaoka et al., (2005) Nature 434:243-249).

Accordingly, in some aspects, the disclosure provides mRNA encoding constitutively active forms of human IRF1, IRF3, IRF5, IRF7, IRF8, and IRF9 as immune potentiators. In some aspects, the disclosure provides mRNA encoding constitutively active forms of human IRF3 and/or IRF7.

During innate immune responses, IRF-3 plays a critical role in the early induction of type I IFNs. The IRF3 transcription factor is constitutively expressed and shuttles between the nucleus and cytoplasm of cells in latent form, with a predominantly cytosolic localization prior to phosphorylation (Hiscott (2007) J Biol Chem 282 (21): 15325-15329; Kumar et al., (2000) Mol Cell Biol 20(11):4159-4168). Upon phosphorylation of serine residues at the C-terminus by TBK-1 (TANK binding kinase 1; also known as T2K and NAK) and/or IKKε (inducible IκB kinase; also known as IKKi), IRF3 translocates from the cytoplasm into the nucleus (Fitzgerald et al., (2003) Nat Immuno 4(5):491-496; Sharma et al., (2003) Science 300:1148-1151; Hemmi et al., (2004) J Exp Med 199:1641-1650). The transcriptional activity of IRF3 is mediated by these phosphorylation and translocation events. A model for IRF3 activation proposes that C-terminal phosphorylation induces a conformational change in IRF3 that promotes homo- and/or heterodimerization (e.g. with IRF7; see Honda et al., (2006) Immunity 25(3):346-360), nuclear localization, and association with the transcriptional co-activators CBP and/or p300 (Lin et al., (1999) Mol Cell Biol 19(4):2465-2474). While inactive IRF3 constitutively shuttles into and out of the nucleus, phosphorylated IRF3 proteins remain associated with CBP and/or p300, are retained in the nucleus, and induce transcription of IFN and other genes (Kumar et al., (2000) Mol Cell Biol 20(11):4159-4168).

In contrast to IRF3, IRF7 exhibits a low expression level in most cells, but is strongly induced by type I IFN-mediated signaling, supporting the notion that IRF3 is primarily responsible for the early induction of IFN genes and that IRF7 is involved in the late induction phase (Sato et al., (2000) Immunity 13 (4): 539-548). Ligand-binding to the type I IFN receptor results in the activation of a heterotrimeric transcriptional activator, termed IFN-stimulated gene factor 3 (ISGF3), which consists of IRF9, STAT1, and STAT2, and is responsible for the induction of the IRF7 gene (Marie et al., (1998) EMBO J 17 (22): 6660-6669). Like IRF3, IRF7 can partition between cytoplasm and nucleus after serine phosphorylation of its C-terminal region, allowing its dimerization and nuclear translocation. IRF7 forms a homodimer or a heterodimer with IRF3, and each of these different dimers differentially acts on the type I IFN gene family members. IRF3 is more potent in activating the IFN-β gene than the IFN-α genes, whereas IRF7 efficiently activates both IFN-α and IFN-β genes (Marie et al., (1998) EMBO J 17 (22): 6660-6669).

Provided herein are mRNAs encoding constitutively active forms of IRF3 and IRF7 including mutant human IRF3 and mutant human IRF7 isoforms for use as immune potentiators as described herein. mRNAs encoding constitutively active forms of IRF3 and IRF7, including mutant human IRF3 and IRF7 isoforms are set forth in the Sequence Listing herein. The amino acid residue numbering for mutant human IRF3 polypeptides used herein corresponds to that used for the 427 amino acid residue wild type human IRF3 (isoform 1) available in the art as Genbank Accession Number NP_001562. The amino acid residue numbering for mutant human IRF7 polypeptides used herein corresponds to that used for the 503 amino acid residue wild type human IRF7 (isoform a) available in the art as Genbank Accession Number NP_001563.

Accordingly, in some aspects, the disclosure provides a mRNA encoding a mutant human IRF3 protein that is constitutively active, e.g., having a mutation at amino acid residue 396, such as an amino acid substitution, such as a S396D mutation, for example as set forth in the amino acid sequence of SEQ ID NO: 12 or encoded by the nucleotide sequence shown in SEQ ID NO: 211 or SEQ ID NO: 1463. In other aspects, the mRNA construct encodes a constitutively active mouse IRF3 polypeptide comprising an S396D mutation, for example as set forth in the amino acid sequence of SEQ ID NO: 11 or encoded by the nucleotide sequence shown in 210 or SEQ ID NO: 1452.

In other aspects, the disclosure provides a mRNA encoding a mutant human IRF7 protein that is constitutively active. In one aspect, the disclosure provides a mRNA encoding a constitutively active IR7 protein comprising one or more point mutations (amino acid substitutions compared to wild-type). In other aspects, the disclosure provides a mRNA encoding a constitutively active IR7 protein comprising a truncated form of the protein (amino acid deletions compared to wild-type). In yet other aspects, the disclosure provides a mRNA encoding a constitutively active IR7 protein comprising a truncated form of the protein that also includes one or more point mutations (a combination of amino acid deletions and amino acid substitutions compared to wild-type).

The wild-type amino acid sequence of human IRF7 (isoform a) is set forth in SEQ ID NO: 13, encoded by the nucleotide sequence shown in SEQ ID NO: 212 or SEQ ID NO: 1454. A series of constitutively active forms of human IRF7 were prepared comprising point mutations, deletions, or both, as compared to the wild-type sequence. In one aspect, the disclosure provides an immune potentiator mRNA construct encoding a constitutively active IRF7 polypeptide comprising one or more of the following mutations: S475D, S476D, S477D, S479D, L480D, S483D and S487D, and combinations thereof. In other aspects, the disclosure provides a mmRNA encoding a constitutively active IRF7 polypeptide comprising mutations S477D and S479D, as set forth in the amino acid sequence of SEQ ID NO: 14, encoded by the nucleotide sequence shown in SEQ ID NO: 213 or SEQ ID NO: 1455. In another aspect, the disclosure provides a mRNA encoding a constitutively active IRF7 polypeptide comprising mutations S475D, S477D and L480D, as set forth in the amino acid sequence of SEQ ID NO: 15, encoded by the nucleotide sequence shown in SEQ ID NO: 214 or SEQ ID NO: 1456. In other aspects, the disclosure provides a mRNA encoding a constitutively active IRF7 polypeptide comprising mutations S475D, S476D, S477D, S479D, S483D and S487D, as set forth in the amino acid sequence of SEQ ID NO: 16, encoded by the nucleotide sequence shown in SEQ ID NO: 215 or SEQ ID NO: 1457. In another aspect, the disclosure provides a mRNA encoding a constitutively active IRF7 polypeptide comprising a deletion of amino acid residues 247-467 (i.e., comprising amino acid residues 1-246 and 468-503), as set forth in the amino acid sequence of SEQ ID NO: 17, encoded by the nucleotide sequence shown in SEQ ID NO: 216 or SEQ ID NO: 1458. In yet other aspects, the disclosure provides a mRNA encoding a constitutively active IRF7 polypeptide comprising a deletion of amino acid residues 247-467 (i.e., comprising amino acid residues 1-246 and 468-503) and further comprising mutations S475D, S476D, S477D, S479D, S483D and S487D, as set forth in the amino acid sequence of SEQ ID NO: 18, encoded by the nucleotide sequence shown in SEQ ID NO: 217 or SEQ ID NO: 1459.

In other aspects, the disclosure provides a mRNA encoding a truncated IRF7 inactive “null” polypeptide construct comprising a deletion of residues 152-246 (i.e., comprising amino acid residues 1-151 and 247-503), as set forth in the amio acid sequence of SEQ ID NO: 19, encoded by the nucleotide sequence shown in SEQ ID NO: 218 or SEQ ID NO: 1460 (used, for example, for control purposes). In other aspects, the disclosure provides a mRNA encoding a truncated IRF7 inactive “null” polypeptide construct comprising a deletion of residues 1-151 (i.e., comprising amino acid residues 152-503), as set forth in the amino acid sequence of SEQ ID NO: 20, encoded by the nucleotide sequence shown in SEQ ID NO: 219 or SEQ ID NO: 1461 (used, for example, for control purposes).

Additional Immune Potentiator mRNAs that Activate Type I IFN

In addition to the STING and IRF mRNA constructs described above, the disclosure provides mRNA constructs encoding additional components of the Type I IFN signaling pathway that can be use as immune potentiators to enhance immune responses through activation of the Type I IFN signaling pathway. For example, in one embodiment, the immune potentiator mRNA construct encodes a MyD88 protein. MyD88 is known in the art to signal upstream of IRF7. In one aspect, the disclosure provides a mmRNA encoding a constitutively active MyD88 protein, such as mutant MyD88 protein having one or more point mutations. In one aspect, the disclosure provides a mRNA encoding a mutant human or mouse MyD88 protein having a L265P substitutions, as set forth in SEQ ID NOs: 134 (encoded by the nucleotide sequence shown in SEQ ID NO: 1409 or SEQ ID NO: 1480) and 135, respectively.

In another aspect, an immune potentiator mRNA construct encodes a MAVS (mitochondrial antiviral signaling) protein. MAVS is known in the art to signal upstream of IRF3/IRF7. MAVS has been demonstrated to be important in the protective interferon response to double-stranded RNA viruses. For example, rotavirus-infected mice lacking MAVS produce significantly less IFN-β and increased amounts of virus than mice with MAVS (Broquet, A. H. et al. (2011) J. Immunol. 186:1618-1626). Moreover, RIG-1 or MDA5 signaling through MAVS has been shown to be required for activation of IFN-β production by rotavirus-infected cells (Broquet et al., ibid). MAVS has also been shown to be critical for Type I interferon responses to Coxsackie B virus, mediated together with MDA5 (Wang, J. P. et al. (2010) J. Virol. 84:254-260). Still further, it has been shown that although distinct classes of receptors are responsible for RNA and DNA sensing in cells, the downstream signaling components are physically and functionally interconnected and there is cross-talk between RIG-1/MAVS RNA sensing and cGAS-STING DNA sensing pathways in potentiating efficient antiviral responses, including interferon responses (Zevini, A. et al. (2017) Trends Immunol. 38:194-205). In one aspect, the disclosure encompasses an mRNA encoding a constitutively active MAVS protein, such as mutant MAVS protein having one or more point mutations. In another aspect, the disclosure encompasses a wild-type MAVS protein that is overexpressed. In one aspect, the disclosure provides an mRNA encoding a MAVS protein as shown in SEQ ID NO: 1387. An exemplary nucleotide sequence encoding the MAVS protein of SEQ ID NO: 1387 is shown in SEQ ID NO: 1413 and SEQ ID NO: 1484.

In another aspect, an immune potentiator mRNA construct encodes a TRAM (TICAM2) protein. TRAM is known in the art to signal upstream of IRF3. In one aspect, the disclosure encompasses a mmRNA encoding a constitutively active TRAM protein, such as mutant TRAM protein having one or more point mutations. In another aspect, the disclosure encompasses a wild-type TRAM protein that is overexpressed. In one aspect, the disclosure provides an mRNA encoding a mouse TRAM protein as shown in SEQ ID NO: 136. An exemplary nucleotide sequence encoding the TRAM protein of SEQ ID NO: 136 is shown in SEQ ID NO: 1410 or SEQ ID NO: 1481.

In yet other aspects, the disclosure provides an immune potentiator mRNA construct encoding a TANK-binding kinase 1 (TBK1) or an inducible IκB kinase (IKKi, also known as IKKε), including constitutively active forms of TBK1 or IKKi, as immune potentiators. TBK1 and IKKi have been demonstrated to be components of the virus-activated kinase that phosphorylates IRF3 and IRF7, thus acting upstream from IRF3 and IRF7 in the Type I IFN signaling pathway (Sharma, S. et al. (2003) Science 300:1148-1151). TBK1 and IKKi are involved in the phosphorylation and activation of transcription factors (e.g. IRF3/7 & NF-κB) that induce expression of type I IFN genes as well as IFN-inducible genes (Fitzgerald, K. A. et al., (2003) Nat Immunol 4 (5): 491-496).

Accordingly, in one aspect, the disclosure provides an immune potentiator mRNA construct that encodes a TBK1 protein, including a constitutively active form of TBK1, including mutant human TBK1 isoforms. In yet other aspects, an immune potentiator mRNA construct encodes a IKKi protein, including a constitutively active form of IKKi, including mutant human IKKi isoforms.

Immune Potentiators mRNAs that Stimulate Inflammatory Responses

In other aspects, the disclosure provides immune potentiator mRNA constructs that enhance an immune response by stimulating an inflammatory response. Non-limiting examples of agents that stimulate an inflammatory response include STAT1, STAT2, STAT4 and STAT6. Accordingly, the disclosure provides an immune potentiator mRNA construct encoding one or a combination of these inflammation-inducing proteins, including a constitutively active form.

Provided herein are mRNAs encoding constitutively active forms of STAT6, including mutant human STAT6 isoforms for use as immune potentiators as described herein. mRNAs encoding constitutively active forms of STAT6, including mutant human STAT6 isoforms are set forth in the Sequence Listing herein. The amino acid residue numbering for mutant human STAT6 polypeptides used herein corresponds to that used for the 847 amino acid residue wild type human STAT6 (isoform 1) available in the art as Genbank Accession Number NP_001171550.1.

In one embodiment, the disclosure provides a mRNA construct encoding a constitutively active human STAT6 construct comprising one or more amino acid mutations selected from the group consisting of S407D, V547A, T548A, Y641F, and combinations thereof. In another embodiment, the mRNA construct encodes a constitutively active human STAT6 construct comprising V547A and T548A mutations, such as the sequence shown in SEQ ID NO: 137. In another embodiment, the mRNA construct encodes a constitutively active human STAT6 construct comprising a S407D mutation, such as the sequence shown in SEQ ID NO: 138. In another embodiment, the mRNA construct encodes a constitutively active human STAT6 construct comprising S407D, V547A and T548A mutations, such as the sequence shown in SEQ ID NO: 139. In another embodiment, the mRNA construct encodes a constitutively active human STAT6 construct comprising V547A, T548A and Y641F mutations, such as the sequence shown in SEQ ID NO: 140.

Immune Potentiator mRNAs that Stimulate NFkB Signaling

In other aspects, the disclosure provides immune potentiator mRNA constructs that enhance an immune response by stimulating NFkB signaling, which is known to be involved in stimulation of immune responses. Non-limiting examples of proteins that stimulate NFkB signaling include STING, c-FLIP, IKKβ, RIPK1, Btk, TAK1, TAK-TAB1, TBK1, MyD88, IRAK1, IRAK2, IRAK4, TAB2, TAB3, TRAF6, TRAM, MKK3, MKK4, MKK6 and MKK7. Accordingly, an immune potentiator mRNA construct of the present disclosure can encode any of these NFkB pathway-inducing proteins, for example in a constitutively active form.

Suitable STING constructs that can serve as immune potentiator mRNA constructs that enhance an immune response by stimulating NFkB signaling are described above in the subsection on immune potentiator mRNA constructs that activate Type I IFN.

Suitable MyD88 constructs that can serve as immune potentiator mRNA constructs that enhance an immune response by stimulating NFkB signaling are described above in the subsection on immune potentiator mRNA constructs that activate Type I IFN.

In one embodiment, the disclosure provides an immune potentiator mRNA construct that activates NFkB signaling encoding a c-FLIP (cellular caspase 8 (FLICE)-like inhibitory protein) protein (also known in the art as CASP8 and FADD-like apoptosis regulator), including a constitutively active c-FLIP. Provided herein are mmRNAs encoding constitutively active forms of c-FLIP, including mutant human c-FLIP isoforms for use as immune potentiators as described herein. mmRNAs encoding constitutively active forms of c-FLIP, including mutant human c-FLIP isoforms are set forth in the Sequence Listing herein. The amino acid residue numbering for mutant human c-FLIP polypeptides used herein corresponds to that used for the 480 amino acid residue wild type human c-FLIP (isoform 1) available in the art as Genbank Accession Number NP_003870.

In one embodiment, the mRNA encodes a c-FLIP long (L) isoform comprising two DED domains, a p20 domain and a p12 domain, such as having the sequence shown in SEQ ID NO: 141. In another embodiment, the mRNA encodes a c-FLIP short(S) isoform, encoding amino acids 1-227, comprising two DED domains, such as having the sequence shown in SEQ ID NO: 142. In another embodiment, the mRNA encodes a c-FLIP p22 cleavage product, encoding amino acids 1-198, such as having the sequence shown in SEQ ID NO: 143. In another embodiment, the mRNA encodes a c-FLIP p43 cleavage product, encoding amino acids 1-376, such as having the sequence shown in SEQ ID NO: 144. In another embodiment, the mRNA encodes a c-FLIP p12 cleavage product, encoding amino acids 377-480, such as having the sequence shown in SEQ ID NO: 145. Exemplary nucleotide sequences encoding the c-FLIP proteins discussed above are shown in SEQ ID NOs: 1398-1402 and 1469-1473.

In another embodiment, an immune potentiator mRNA construct that activates NFkB signaling encodes a constitutively active IKKα mRNA construct or a constitutively active IKKβ mRNA construct. In one embodiment, the constitutively active human IKKβ polypeptide comprises S177E and S181E mutations, such as the sequence shown in SEQ ID NO: 146. In another embodiment, the constitutively active human IKKβ polypeptide comprises S177A and S181A mutations, such as the sequence shown in SEQ ID NO: 147. In another embodiment, the mRNA construct encodes a constitutively active mouse IKKβ polypeptide. In one embodiment, the constitutively active mouse IKKβ polypeptide comprises S177E and S181E mutations, such as the sequence shown in SEQ ID NO: 148. In another embodiment, the constitutively active mouse IKKβ polypeptide comprises S177A and S181A mutations, such as the sequence shown in SEQ ID NO: 149. An exemplary nucleotide sequence encoding the protein of SEQ ID NO: 146 is shown in SEQ ID NO: 1414 and SEQ ID NO: 1485. In another embodiment, the mRNA construct encodes a constitutively active human or mouse IKKα polypeptide comprising a PEST mutation, such as having a sequence as shown in SEQ ID NOs: 150 (human) (encoded by the nucleotide sequence shown in SEQ ID NO: 151 or SEQ ID NO: 28) or 154 (mouse) (encoded by the nucleotide sequence shown in SEQ ID NO: 155 or SEQ ID NO: 1429). In another embodiment, the mRNA construct encodes a constitutively active human or mouse IKKβ polypeptide comprising a PEST mutation, such as having the sequence shown in SEQ ID NOs: 152 (human) (encoded by the nucleotide sequence shown in SEQ ID NO: 153 or SEQ ID NO: 1397) or 156 (mouse) (encoded by the nucleotide sequence shown in SEQ ID NO: 157 or SEQ ID NO: 1430).

In another embodiment, the disclosure provides an immune potentiator mRNA construct that activates NFkB signaling encoding a receptor-interacting protein kinase 1 (RIPK1) protein. Structure of DNA constructs encoding RIPK1 constructs that induce immunogenic cell death are described in the art, for example, Yatim, N. et al. (2015) Science 350:328-334 or Orozco, S. et al. (2014) Cell Death Differ. 21:1511-1521, and can be used in the design of suitable RNA constructs that are shown herein to also active NFkB signaling (see Examples). In one embodiment, the mRNA construct encodes RIPK1 amino acids 1-555 of a human or mouse RIPK1 polypeptide as well as an IZ domain, such as having the sequence shown in SEQ ID N: 158 (human) or 161 (mouse). In one embodiment, the mRNA construct encodes RIPK1 amino acids 1-555 of a human or mouse RIPK1 polypeptide as well as EE and DM domains, such as having the sequence shown in SEQ ID N: 159 (human) or 162 (mouse). In one embodiment, the mRNA construct encodes RIPK1 amino acids 1-555 of a human or mouse RIPK1 polypeptide as well as RR and DM domains, such as having the sequence shown in SEQ ID N: 160 (human) or 163 (mouse). Exemplary nucleotide sequences encoding the RIPK1 polypeptides described above are shown in SEQ ID NOs: 1403-1408 and 1474-1479.

In yet another embodiment, an immune potentiator mRNA construct that activates NFkB signaling encodes a Btk polypeptide, such as a mutant Btk polypeptide such as a Btk (E41K) polypeptide (e.g., encoding an ORF amino acid sequence shown in SEQ ID NO: 173).

In yet another embodiment, an immune potentiator mRNA construct that activates NFkB signaling encodes a TAK1 protein, such as a constitutively active TAK1.

In yet another embodiment, an immune potentiator mRNA construct that activates NFkB signaling encodes a TAK-TAB1 protein, such as a constitutively active TAK-TAB1. In one embodiment, an immune potentiator mRNA construct encodes a human TAK-TAB1 protein, such as having the sequence shown in SEQ ID NO: 164. An exemplary nucleotide sequence encoding the TAK-TAB1 protein of SEQ ID NO: 164 is shown in SEQ ID NO: 1411 or SEQ ID NO: 1482.

Immune Potentiator mRNAs Encoding Intracellular Adaptor Proteins

In one embodiment, the polypeptide encoded by the immune potentiator mRNA construct is an intracellular adaptor protein. Intracellular adaptors (also referred to as signal transducing adaptor proteins) are proteins that are accessories to main proteins in a signal transduction pathway. Adaptor proteins contain a variety of protein-binding modules that link protein-binding partners together and facilitate the creation of larger signaling complexes. These proteins tend to lack any intrinsic enzymatic activity themselves but instead mediate specific protein-protein interactions that drive the formation of protein complexes.

In one embodiment, the intracellular adaptor protein stimulates a Type I IFN response. In another embodiment, the intracellular adaptor protein stimulates an NFkB-mediated proinflammatory response.

In one embodiment, the intracellular adaptor protein is a STING protein, such as a constitutively active form of STING polypeptide, including mutant human STING isoforms. STING has been established in the art as an endoplasmic reticulum adaptor that facilitates innate immune signaling and has been shown to activate both NFkB-mediated and IRF3/IRF7-mediated transcription pathways to induce expression of Type I IFNs (see e.g., Ishikawa, H. and Barber, G. H. (2008) Nature 455:674-678). For example, STING acts as an adaptor protein in the activation of TBK1 (upstream of NFkB-mediated and IRF3/IRF-mediated transcription) following activation of cGAS and IFI16 by double-stranded DNA (e.g., viral DNA). Suitable mRNA constructs encoding STING are described in detail above in the section of immune potentiators that activate Type I interferon.

In another embodiment, the intracellular adaptor protein is a MAVS protein, such as a constitutively active form of MAVS polypeptide, including mutant human MAVS isoforms. MAVS is also known in the art as VISA (virus-induced signaling adaptor), IPS-1 or Cardif. MAVS has been established in the art to act as an intracellular adaptor protein in the activation of TBK1 (upstream of NFkB-mediated and IRF3/IRF-mediated transcription) following activation of the cytoplasmic RNA helicases RIG-1 and MDA5 by double stranded RNA (e.g., double-stranded RNA viruses). Suitable mRNA constructs encoding MAVS are described in detail above in the subsection of immune potentiators that activate Type I interferon.

In another embodiment, the intracellular adaptor protein is a MyD88 protein, such as a constitutively active form of MyD88 polypeptide, including mutant human MyD88 isoforms. MyD88 has been established in the art as an intracellular adaptor protein that is used by TLRs to activate Type I IFN responses and NFkB-mediated proinflammatory responses (see e.g., O'Neill, L. A. et al. (2003) J. Endotoxin Res. 9:55-59). Suitable mRNA constructs encoding MyD88 are described in detail above in the subsection on immune potentiators that activate Type I IFN responses.

Immune Potentiator mRNAs Encoding Intracellular Signalling Proteins

In another embodiment, the polypeptide encoded by the immune potentiator mRNA construct is an intracellular signaling protein. As used herein, an “intracellular signaling protein” refers to a protein involved in a signal transduction pathway and typically has enzymatic activity (e.g., kinase activity). In one embodiment, the polypeptide is an intracellular signaling protein of a TLR signaling pathway (i.e., the polypeptide is an intracellular molecule that functions in the transduction of TLR-mediated signaling but is not a TLR itself). In one embodiment, the intracellular signalling protein stimulates a Type I IFN response. In another embodiment, the intracellular signalling protein stimulates an NFkB-mediated proinflammatory response. Non-limiting examples of intacellular signalling proteins include MyD88, IRAK 1, IRAK2, IRAK4, TRAF3, TRAF6, TAK1, TAB2, TAB3, TAK-TAB1, MKK3, MKK4, MKK6, MKK7, IKKα, IKKβ, TRAM, TRIF, RIPK1, and TBK1. Specific examples of intracellular signaling proteins are described in the subsections on immune potentiators that activate Type I interferon or activate NFκB signaling.

Immune Potentiator mRNAs Encoding Transcription Factors

In another embodiment, the polypeptide encoded by the immune potentiator mRNA construct is a transcription factor. A transcription factor contains at least one sequence-specific DNA binding domain and functions to regulate the rate of transcription of a gene(s) to mRNA. In one embodiment, the transcription factor stimulates a Type I IFN response. In another embodiment, the transcription factor stimulates an NFκB-mediated proinflammatory response. Non-limiting examples of transcription factors include IRF3 or IRF7. Specific examples of IRF3 and IRF7 constructs are described in the subsection on immune potentiators that activate Type I interferon.

Immune Potentiator mRNAs Encoding Polypeptides Involved in Necroptosis or Necroptosome Formation

In another embodiment, the polypeptide encoded by the immune potentiator mRNA construct is involved in necroptosis or necroptosome formation. A polypeptide is “involved in” necroptosis or necroptosome formation if the protein mediates necroptosis itself or participates with additional molecules in mediating necroptosis and/or in necroptosome formation. Non-limiting examples of polypeptides involved in necroptosis or necroptosome formation include MLKL, RIPK1, RIPK3, DIABLO and FADD.

Suitable mRNA constructs encoding RIPK1 are described in detail above in the section of immune potentiators that activate NFκB signaling.

In one embodiment, the polypeptide encoded by the immune potentiator mRNA construct is mixed lineage kinase domain-like protein (MLKL). MLKL constructs induce necroptotic cell death, characterized by release of DAMPs. In one embodiment, the mRNA construct encodes amino acids 1-180 of human or mouse MLKL. Non-limiting examples of mRNA constructs encoding MLKL, or an immunogenic cell death-inducing fragment thereof, encode amino acids 1-180 of human or mouse MLKL comprising the amino sequences shown in SEQ ID NOs: 1327 and 1328, respectively. An exemplary nucleotide sequence encoding the MLKL protein of SEQ ID NO: 1327 is shown in SEQ ID NO: 1412 and SEQ ID NO: 1483.

In another embodiment, the polypeptide encoded by the immune potentiator mRNA construct is receptor-interacting protein kinase 3 (RIPK3). In one embodiment, the mRNA construct encodes a RIPK3 polypeptide that multimerize with itself (homo-oligomerization). In one embodiment, the mRNA construct encodes a RIPK3 polypeptide that dimerizes with RIPK1. In one embodiment, the mRNA construct encodes the kinase domain and the RHIM domain of RIPK3. In one embodiment, the mRNA construct encodes the kinase domain of RIPK3, the RHIM domain of RIPK3 and two FKBP(F>V) domains. In one embodiment, the mRNA construct encodes a RIPK3 polypeptide (e.g., comprising the kinase domain and the RHIM domain of RIPK3) and an IZ domain (e.g., an IZ trimer). In one embodiment, the mRNA construct encodes a RIPK3 polypeptide (e.g., comprising the kinase domain and the RHIM domain of RIPK3) and one or more EE or RR domains (e.g., 2xEE domains, or 2xRR domains). Additionally, the structure of DNA constructs encoding RIPK3 constructs that induce immunogenic cell death are described further in, for example, Yatim, N. et al. (2015) Science 350:328-334 or Orozco, S. et al. (2014) Cell Death Differ. 21:1511-1521, and can be used in the design of suitable RNA constructs. Non-limiting examples of mRNA constructs encoding RIPK3 comprise an ORF having any of the amino acid sequences shown in SEQ ID NOs: 1329-1344 and 1379. An exemplary nucleotide sequence encoding the RIPK3 polypeptide of SEQ ID NO: 1339 is shown in SEQ ID NO: 1415 and SEQ ID NO: 1486.

In another embodiment, an immune potentiator mRNA construct encodes direct IAP binding protein with low pI (DIABLO) (also known as SMAC/DIABLO). As described in the examples herein, DIABLO constructs induce release of cytokines. In one embodiment, the disclosure provides a mRNA construct encoding a wild-type human DIABLO Isoform 1 sequence, such as having the sequence shown in SEQ ID NO: 165 (corresponding to the 239 amino acid human DIABLO isoform 1 precursor disclosed in the art as Genbank Accession No. NP_063940.1). In another embodiment, the mRNA construct encodes a human DIABLO Isoform 1 sequence comprising an S126L mutation, such as having the sequence shown in SEQ ID NO: 166. In another embodiment, the mRNA construct encodes amino acids 56-239 of human DIABLO Isoform 1, such as having the sequence shown in SEQ ID N: 167. In another embodiment, the mRNA construct encodes amino acids 56-239 of human DIABLO Isoform 1 and comprises an S126L mutation, such as having the sequence shown in SEQ ID NO: 168. In another embodiment, the mRNA construct encodes a wild-type human DIABLO Isoform 3 sequence, such as having the sequence shown in SEQ ID NO: 169 (corresponding to the 195 amino acid human DIABLO isoform 3 disclosed in the art as Genbank Accession No. NP_001265271.1). In another embodiment, the mRNA construct encodes a human DIABLO Isoform 3 sequence comprising an S82L mutation, such as having the sequence shown in SEQ ID NO: 170. In another embodiment, the mRNA construct encodes amino acids 56-195 of human DIABLO Isoform 3, such as having the sequence shown in SEQ ID NO: 171. In another embodiment, the mRNA construct encodes amino acids 56-195 of human DIABLO Isoform 3 and comprises an S82L mutation, such as having the sequence shown in SEQ ID NO: 172. An exemplary nucleotide sequence encoding the DIABLO polypeptide of SEQ ID NO: 169 is shown in SEQ ID NO: 1416 and SEQ ID NO: 1487.

In another embodiment, the polypeptide encoded by the immune potentiator mRNA construct is FADD (Fas-associated protein with death domain). Non-limiting examples of mRNA constructs encoding FADD comprise an ORF having any of the amino acid sequences shown in SEQ ID NOs: 1345-1351. Exemplary nucleotide sequences encoding the FADD proteins are shown in SEQ ID NOs: 1417-1422 and 1488-1493.

Immune Potentiator mRNAs Encoding Polypeptides Involved in Pyroptosis or Inflammasome Formation

In another embodiment, the polypeptide encoded by the immune potentiator mRNA construct is involved in pyroptosis or inflammasome formation. A polypeptide is “involved in” pyroptosis or inflammasome formation if the protein mediates pyroptosis itself or participates with additional molecules in mediating pyroptosis and/or in inflammasome formation. Non-limiting examples of polypeptides involved in pyroptosis or inflammasome formation include caspase 1, caspase 4, caspase 5, caspase 11, GSDMD, NLRP3, Pyrin domain and ASC/PYCARD.

In on embodiment, the polypeptide encoded by the immune potentiator mRNA construct is caspase 1. In one embodiment, the caspase 1 polypeptide is a self-activating caspase-1 polypeptide (e.g, encoding any of the ORF amino acid sequences shown in SEQ ID NOs: 175-178), which can promote cleavage of pro-IL1β and pro-IL18 to their respective mature forms.

In another embodiment, the polypeptide encoded by the immune potentiator mRNA construct is caspase-4 or caspase-5 or caspase-11. In various embodiments, the caspase-4, -5 or -11 construct can encode (i) full-length wild-type caspase-4, caspase-5 or caspase-11; (ii) full-length caspase-4, -5 or -11 plus an IZ domain; (iii) N-terminally deleted caspase-4, -5 or -11 plus an IZ domain; (iv) full-length caspase-4, -5 or -11 plus a DM domain; or (v) N-terminally deleted caspase-4, -5 or -11 plus a DM domain. Examples of N-terminally deleted forms of caspase-4 and caspase-11 contain amino acid residues 81-377. An example of an N-terminally deleted form of caspase-5 contains amino acid residues 137-434. Non-limiting examples of mRNA constructs encoding caspase-4 comprise an ORF having any of the amino acid sequences shown in SEQ ID NOs: 1352-1356. Non-limiting examples of mRNA constructs encoding caspase-5 comprise an ORF having any of the amino acid sequences shown in SEQ ID NOs: 1357-1361. Non-limiting examples of mRNA constructs encoding caspase-11 comprise an ORF having any of the amino acid sequences shown in SEQ ID NOs: 1362-1366.

In one embodiment, the polypeptide encoded by the immune potentiator mRNA construct is gasdermin D (GSDMD). In one embodiment, the mRNA construct encodes a wild-type human GSDMD sequence. In another embodiment, the mRNA construct encodes amino acids 1-275 of human GSDMD. In another embodiment, the mRNA construct encodes amino acids 276-484 of human GSDMD. In another embodiment, the mRNA construct encodes wild-type mouse GSDMD. In another embodiment, the mRNA construct encodes amino acids 1-276 of mouse GSDMD. In another embodiment, the mRNA construct encodes amino acids 277-487 of mouse GSDMD. Non-limiting examples of mRNA constructs encoding GSDMD comprise an ORF having any of the amino acid sequences shown in SEQ ID NOs: 1367-1372.

In another embodiment, the polypeptide encoded by the immune potentiator mRNA construct is NLRP3. Non-limiting examples of mRNA constructs encoding NLRP3 encode the ORF amino acid sequences shown in SEQ ID NOs: 1373 or 1374.

In another embodiment, the polypeptide encoded by the immune potentiator mRNA construct is apoptosis-associated speck-like protein containing a CARD (ASC/PYCARD), or a fragment thereof, such as a domain. In one embodiment, the polypeptide is a Pyrin B30.2 domain. In another embodiment, the polypeptide is a Pyrin B30.2 domain comprising a V726A mutation. Non-limiting examples of mRNA constructs encoding a Pyrin B30.2 domain encode the ORF amino acid sequences shown in SEQ ID NOs: 1375 or 1376. Non-limiting examples of mRNA constructs encoding ASC encode the ORF amino acid sequences shown in SEQ ID NOs: 1377 or 1378.

Additional Immune Potentiator mRNAs

The present disclosure provides additional immune potentiator mRNA constructs. In some embodiments, the immune potentiator mRNA construct encodes a SOC3 polypeptide (e.g., encoding an ORF amino acid sequence shown in SEQ ID NO: 174).

In yet other embodiments, an immune potentiator mRNA construct encodes a protein that modulates dendritic cell (DC) activity, such as stimulating DC production, activity or mobilization. A non-limiting example of a protein that stimulates DC mobilization is FLT3. Accordingly, in one embodiment, the immune potentiator mRNA construct encodes a FLT3 protein.

An immune potentiator mRNA construct typically comprises, in addition to the polypeptide-encoding sequences, other structural properties as described herein for mRNA constructs (e.g., modified nucleobases, 5′ cap, 5′ UTR, 3′ UTR, miR binding site(s), polyA tail, as described herein). Suitable mRNA construct components are as described herein.

Antigens of Interest Including mRNAs

The immune potentiators mRNAs of the disclosure are useful in combination with any type of antigen for which enhancement of an immune response is desired, including with mRNA sequences encoding at least one antigen of interest (on either the same or a separate mRNA construct) to enhance immune responses against the antigen of interest, such as a tumor antigen or a pathogen antigen. Thus, the immune potentiator mRNAs of the disclosure enhance, for example, mRNA vaccine responses, thereby acting as genetic adjuvants. In one embodiment, the antigen(s) of interest is a tumor antigen. In another embodiment, the antigen(s) of interest is a pathogen antigen. In various embodiments, the pathogen antigen(s) can be from a pathogen selected from the group consisting of viruses, bacteria, protozoa, fungi and parasites.

In one embodiment, the antigen is an endogenous antigen, such as a tumor antigen or pathogen antigen released in situ. Alternatively, the antigen is an exogenous antigen. An exogenous antigen can be coadministered with the immune potentiator mRNA construct or, alternatively, can be administered before or after the immune potentiator mRNA construct. An exogenous antigen can be coformulated with an immune potentiator mRNA construct or, alternatively, can be separately formulated from the immune potentiator mRNA construct. In one embodiment, an exogenous antigen is encoded by an mRNA construct (e.g., mmRNA construct), either the same or a different mRNA construct as that encoding the immune potentiator. In other embodiments, the antigen can be, for example, a protein, a peptide, a glycoprotein, a polysaccharide or a lipid.

In one embodiment, the antigen(s) of interest is a tumor antigen. In one embodiment, the tumor antigen comprises a tumor neoepitope, e.g., mutant peptide from a tumor antigen. In one embodiment, the tumor antigen is a Ras antigen. A comprehensive survey of Ras mutations in cancer has been described in the art (Prior, I. A. et al. (2012) Cancer Res. 72:2457-2467). Accordingly, a Ras amino acid sequence comprising at least one mutation associated with cancer can be used as an antigen of interest. In one embodiment, the tumor antigen is a mutant KRAS antigen. Mutant KRAS antigens have been implicated in acquired resistance to certain therapeutic agents (see e.g., Misale, S. et al. (2012) Nature 486:532-536; Diaz, L. A. et al. (2012) Nature 486:537-540). Furthermore, anti-tumor vaccines comprising at least one mutant RAS peptide and an anti-metabolite chemotherapeutic agent have been described in the art (U.S. Pat. No. 9,757,439, the entire contents of which is expressly incorporated herein by reference). Accordingly, any of the mutant RAS peptides described in U.S. Pat. No. 9,757,439 can be used as an antigen of the disclosure, e.g., in combination with an immune potentiator of the disclosure to thereby enhance anti-tumore immune responses against a Ras tumor antigen.

In one embodiment, a mutant KRAS antigen comprises an amino acid sequence having one or more mutations selected from G12D, G12V, G13D and G12C, and combinations thereof. Non-limiting examples of mutant KRAS antigens include those comprising one or more of the amino acid sequences shown in SEQ ID NOs: 95-106 and 131-132. In one embodiment, the mutant KRAS antigen is one or more mutant KRAS 15mer peptides comprising a mutation selected from G12D, G12V, G13D and G12C, non-limiting examples of which are shown in SEQ ID NO: 95-97. In another embodiment, the mutant KRAS antigen is one or more mutant KRAS 25mer peptides comprising a mutation selected from G12D, G12V, G13D and G12C, non-limiting examples of which are shown in SEQ ID NO: 98-100 and 131. In another embodiment, the mutant KRAS antigen is one or more mutant KRAS 3×15mer peptides (3 copies of the 15mer peptide) comprising a mutation selected from G12D, G12V, G13D and G12C, non-limiting examples of which are shown in SEQ ID NO: 101-103. In another embodiment, the mutant KRAS antigen is one or more mutant KRAS 3×25mer peptides (three copies of the 25mer peptide) comprising a mutation selected from G12D, G12V, G13D and G12C, non-limiting examples of which are shown in SEQ ID NO: 104-106 and 132. In another embodiment, the mutant KRAS antigen is a 100mer concatemer peptide of the 25mer peptides containing the G12D, G12V, G13D and G12C mutations (i.e., a 100mer concatemer of SEQ ID NOs: 98, 99, 100 and 131). Accordingly, in one embodiment, the mutant KRAS antigen comprises an mRNA construct encoding SEQ ID NOs: 98, 99, 100 and 131. Further description of mutant KRAS antigens, amino acid sequences thereof, and mRNA sequences encoding therefor, are disclosed in U.S. Application Ser. No. 62/453,465, the entire contents of which is expressly incorporated herein by reference. In some embodiments, the mutant KRAS antigen is a 100mer concatemer peptide of the 25mer peptides containing the G12D, G12V, G13D and G12C mutations encoded by a nucleotide sequence shown in SEQ ID NO: 1321 or 1322.

In one embodiment, a tumor antigen is encoded by an mRNA construct that also comprises an immune potentiator (i.e., also encodes a polypeptide that enhances an immune response against the tumor antigen). Non-limiting examples of such constructs include the KRAS-STING constructs encoding one of the amino acid sequences shown in SEQ ID NOs: 107-130. Non-limiting examples of nucleotide sequences encoding the KRAS-STING constructs are shown in SEQ ID NOs: 220-223.

In yet another embodiment, the tumor antigen is an oncogenic virus antigen. In one embodiment, the oncogenic virus is human papillomavirus (HPV) and the HPV antigen(s) is an E6 and/or an E7 antigen. Non-limiting examples of HPV E6 antigens include those comprising an amino acid sequence shown in SEQ ID NOs: 36-72. Non-limiting examples of HPV E7 antigens include those comprising an amino acid sequence shown in SEQ ID NOs: 73-94. In other embodiments, the HPV antigen is an E1, E2, E4, E5, L1 or L2 protein, or antigenic peptide sequence thereof. Suitable HPV antigens are described further in PCT Application No. PCT/US2016/058314, the entire contents of which is expressly incorporated herein by reference.

In another embodiment, the tumor antigen is encoded by an mRNA cancer vaccine. Suitable mRNA cancer vaccines are described in detail in PCT Application No. PCT/US2016/044918, the entire contents of which is expressly incorporated herein by reference.

In yet another embodiment, the tumor antigen is an endogenous tumor antigen, such as a tumor antigen that is released upon destruction of tumor cells in situ. It has been established in the art that natural mechanisms exist that results in cell death in vivo leading to release of intracellular components such that an immune response may be stimulated against the intracellular components. Such mechanisms are referred to herein as immunogenic cell death and include necroptosis and pyroptosis. Accordingly, in one embodiment, an immune potentiator mRNA construct of the disclosure is administered to a tumor-bearing subject under conditions in which endogenous immunogenic cell death is occurring such that one or more endogenous tumor antigens are released, to thereby enhance an immune response against the tumor antigens. In one embodiment, the immune potentiator mRNA construct is administered to a tumor-bearing subject together with a second mRNA construct encoding an “executioner mRNA construct”, which stimulates immunogenic cell death of tumor cells in the subject. Examples of executioner mRNA constructs include those encoding MLKL, RIPK3, RIPK1, DIABLO, FADD, GSDMD, caspase-4, caspase-5, caspase-11, Pyrin, NLRP3 and ASC/PYCARD. Executioner mRNA constructs, and their use in combination with an immune potentiator mRNA construct, are described in further detail in U.S. Application Ser. No. 62/412,933, the entire contents of which is expressly incorporated herein by reference.

In one embodiment, the antigen(s) of interest is a pathogen antigen. In one embodiment, the pathogen antigen comprises a viral antigen. In one embodiment, the viral antigen is a human papillomavirus (HPV) antigen. In one embodiment, the HPV antigen is an E6 or an E7 antigen. Non-limiting examples of HPV E6 antigens include those comprising an amino acid sequence shown in SEQ ID NOs: 36-72. Non-limiting examples of HPV E7 antigens include those comprising an amino acid sequence shown in SEQ ID NOs: 73-94. In other embodiments, the HPV antigen is an E1, E2, E4, E5, L1 or L2 protein, or antigenic peptide sequence thereof. Suitable HPV antigens are described further in PCT Application No. PCT/US2016/058314, the entire contents of which is expressly incorporated herein by reference. In another embodiment, the viral antigen is a herpes simplex virus (HSV) antigen, such as an HSV-1 or HSV-2 antigen. For example, the viral antigen can be an HSV (HSV-1 or HSV-2) glycoprotein B, glycoprotein C, glycoprotein D, glycoprotein E, glycoprotein I, ICP4 or ICP0 antigen. Suitable HSV antigens are described further in PCT Application No. PCT/US2016/058314, the entire contents of which is expressly incorporated herein by reference.

In one embodiment, the pathogen antigen is a bacterial antigen. In one embodiment, the bacterial antigen is a multivalent antigen (i.e., the antigen comprises multiple antigenic epitopes, such as multiple antigenic peptides comprising different epitopes). In one embodiment, the bacterial antigen is a Chlamydia antigen, such as a MOMP, OmpA, OmpL, OmpF or OprF antigen. Suitable Chlamydia antigens are described further in PCT Application No. PCT/US2016/058314, the entire contents of which is expressly incorporated herein by reference.

In one embodiment, a pathogen antigen is encoded by an mRNA construct that also comprises an immune potentiator (i.e., also encodes a polypeptide that enhances an immune response against the tumor antigen).

An mRNA construct encoding an antigen(s) of interest typically comprises, in addition to the antigen-encoding sequences, other structural properties as described herein for mRNA constructs (e.g., modified nucleobases, 5′ cap, 5′ UTR, 3′ UTR, miR binding site(s), polyA tail, as described herein). Suitable mRNA construct components are as described herein.

### Oncoviruses

In one embodiment, an immune potentiator construct is used to enhance an immune response against one or more antigens from an oncogenic virus (oncovirus). Viral infections are the cause of a significant proportion of all human cancers. It has been estimated that approximately 12% of all human cancers worldwide have a viral etiology (Parkin (2006) Int J Cancer 118:3030-3044). The term “oncovirus” refers to any virus with a DNA and/or RNA genome capable of causing cancer and can be used synonymously with the terms “tumor virus” or “cancer virus”. The World Health Organization's International Agency for Research on Cancer (IARC) has recognized seven human oncoviruses as Group 1 Biological carcinogenic agents for which there is “sufficient evidence of carcinogenicity in humans”, including hepatitis B virus (HBV), hepatitis C virus (HCV), Epstein-Barr virus (EBV), high-risk human papillomaviruses (HPVs), human T cell lymphotropic virus type 1 (HTLV-1), human immunodeficiency virus (HIV), and Kaposi's sarcoma herpes virus (KSHV) (Bouvard et al., (2009) Lancet Oncol 10:321-322). Merkel cell polyomavirus (MCV) is a recently discovered oncovirus that is classified by the IARC as a Group 2A Biological carcinogenic agent (Feng et al., (2008) Science 319 (5866): 1096-1100).

The excellent record of safety, effectiveness, and ability to reach economically disadvantaged populations for vaccines targeting pathogenic viruses (e.g. polio, influenza) have prompted efforts to develop and implement prophylactic and therapeutic vaccination strategies targeting oncoviruses (Schiller and Lowy (2010) Ann Rev Microbiol 64:23-41). Accordingly, in one aspect, an immune potentiator construct can be used to enhance an immune response against one or more antigens of interest of an oncogenic virus. For example, an antigen(s) of interest from an oncogenic virus can be encoded by a chemically modified mRNA (mmRNA), provided on the same mmRNA as the immune potentiator construct or provided on a different construct mmRNA construct as the immune potentiator. The immune potentiator and antigen mmRNAs can be formulated (or coformulated) and administered (simultaneously or sequentially) to a subject in need thereof to stimulate an immune response against the oncogenic viral antigen(s) in the subject. Non-limiting examples of oncogenic viruses, and suitable antigens thereof for use in combination with an immune potentiator construct to thereby enhance an immune response against the oncogenic virus, are described further below.

### A. Human Papillomaviruses (HPVs)

In one embodiment, the oncoviral antigen is from human papilloma virus (HPV). Cervical cancer is the fourth most prevalent malignancy affecting women worldwide (Wakeham and Kavanagh (2014) Curr Oncol Rep 16 (9): 402). Infection with human papillomavirus (HPV) is associated with nearly all cases of cervical cancer and is responsible for causing several other cancers including: penile, vaginal, vulval, anal and oropharyngeal (Forman et al., (2012) Vaccine 30 Suppl 5: F12-23; Maxwell et al., (2016) Annu Rev Med 67:91-101). To date, more than 300 papillomaviruses have been identified and sequenced, including over 200 types of HPV, which are categorized according to their oncogenic potential. The association between the development of cervical cancer and infection with “high-risk” HPV types is well-established and provides the rationale for HPV DNA testing during cervical screening and for the development of prophylactic vaccines (Egawa et al., (2015) Viruses 7 (7): 3863-3890). Among high-risk HPV types, HPV16 and HPV18 are the major papillomavirus types responsible for about 70% of cervical cancer cases (Walboomers et al., (1999) J Pathol 189 (1): 12-19; Clifford et al., (2002) Bri J Cancer 88:63-73).

The identification of HPV as the etiological agent of cervical cancer and other orogenital malignancies provided the opportunity to mitigate the morbidity and mortality caused by HPV-associated cancers through vaccination and other therapeutic strategies targeting the HPV infection (zur Hausen (2002) Nat Rev Cancer 2 (5): 342-350). Prophylactic HPV vaccines exist targeting the major capsid protein L1 of the HPV viral particle (Harper et al., (2010) Discov Med 10 (50): 7-17; Kash et al., (2015) J Clin Med 4 (4): 614-633). These vaccines have prevented uninfected people from acquiring HPV infections as well as previously infected patients from being re-infected. However, currently available HPV vaccines are not able to treat or clear established HPV infections and HPV-associated lesions (Ma et al., (2012) Expert Opin Emerg Drugs 17 (4): 469-492). Therapeutic HPV vaccines represent a potential treatment approach to clear existing HPV infections and associated diseases. Unlike prophylactic HPV vaccines, which can generate neutralizing antibodies against viral particles, therapeutic HPV vaccines can stimulate cell-mediated immune responses to specifically target and kill infected cells.

Although many HPV infections remain asymptomatic and are cleared by the immune system, persistent HPV infections can develop, which may further develop into low or high-grade cervical intraepithelial neoplasia and/or cervical carcinoma (Ostor (1993) Int J Gynecol pathol 12 (2): 186-192; Ghittoni et al., (2015) Ecancermedicalscience 9:526). HPV viral DNA integrates into the host's genome in many HPV-associated lesions and cancers. This integration can lead to the deletion of early (E1, E2, E4, and E5) and late (L1 and L2) genes. The deletion of L1 and L2 during the integration process precludes the use of prophylactic vaccines against HPV-associated cancers. Furthermore, E2 is a negative regulator for the HPV oncogenes E6 and E7. The deletion of E2 during integration results in increased expression of E6 and E7 and is thought to contribute to HPV-associated carcinogensis. Oncoproteins E6 and E7 are required for the initiation and upkeep of HPV-associated malignancies and are expressed in transformed cells. Therapeutic HPV vaccines targeting E6 and E7 can circumvent the problem of immune tolerance against self-antigens because these virus encoded oncogenic proteins are foreign proteins to human bodies. For these reasons HPV oncoproteins E6 and E7 serve as an ideal target for therapeutic HPV vaccines.

Accordingly, in one aspect, an immune potentiator construct can be used to enhance an immune response against one or more HPV antigens of interest. For example, an antigen(s) of interest from HPV can be encoded by a chemically modified mRNA (mmRNA), provided on the same mmRNA as the immune potentiator construct or provided on a different construct mmRNA construct as the immune potentiator. The immune potentiator and HPV antigen mmRNAs can be formulated (or coformulated) and administered (simultaneously or sequentially) to a subject in need thereof to stimulate an immune response against the HPV antigen in the subject.

In some embodiments, a RNA (e.g., mRNA) vaccine (e.g., comprising an immune potentiator construct and an HPV antigen construct, on the same or different mRNAs) comprises at least one RNA (e.g., mRNA) polynucleotide having an open reading frame encoding at least one HPV antigenic polypeptide or an immunogenic fragment thereof (e.g., an immunogenic fragment capable of inducing an immune response to HPV). In some embodiments, at least one HPV antigenic polypeptide is selected from E1, E2, E4, E5, E6, E7, L1, and L2, and combinations thereof. In some embodiments, the at least one antigenic polypeptide is selected from E1, E2, E4, E5, E6, and E7. In some embodiments, the at least one antigenic polypeptide is E6, E7, or a combination of E6 and E7. In some embodiments, the at least one antigenic polypeptide is L1, L2, or a combination of L1 and L2.

In some embodiments, the at least one antigenic polypeptide is L1. In some embodiments, the L1 protein is obtained from HPV serotypes 6, 11, 16, 18, 31, 33, 35, 39, 30, 45, 51, 52, 56, 58, 59, 68, 73 or 82.

In some embodiments, the at least one antigenic polypeptide is L1, L2 or a combination of L1 and L2, and E6, E7, or a combination of E6 and E7.

In some embodiments, the at least one antigenic polypeptide is from HPV strain HPV type 16 (HPV16), HPV type 18 (HPV18), HPV type 26 (HPV26), HPV type 31 (HPV31), HPV type 33 (HPV33), HPV type 35 (HPV35), HPV type 45 (HPV45), HPV type 51, (HPV51), HPV type 52 (HPV52), HPV type 53 (HPV53), HPV type 56 (HPV56), HPV type 58 (HPV58), HPV type 59 (HPV59), HPV type 66 (HPV66), HPV type 68 (HPV68), HPV type 82 (HPV82), or a combination thereof. In some embodiments, the at least one antigenic polypeptide is from HPV strain HPV16, HPV18, or a combination thereof.

In some embodiments, the at least one antigenic polypeptide is from HPV strain HPV type 6 (HPV6), HPV type 11 (HPV11), HPV type 13 (HPV13), HPV type 40 (HPV40), HPV type 42 (HPV42), HPV type 43 (HPV43), HPV type 44 (HPV44), HPV type 54 (HPV54), HPV type 61 (HPV61), HPV type 70 (HPV70), HPV type 72 (HPV72), HPV type 81, (HPV81), HPV type 89 (HPV89), or a combination thereof.

In some embodiments, the at least one antigenic polypeptide is from HPV strain HPV type 30 (HPV30), HPV type 34 (HPV34), HPV type 55 (HPV55), HPV type 62 (HPV62), HPV type 64 (HPV64), HPV type 67 (HPV67), HPV type 69 (HPV69), HPV type 71 (HPV71), HPV type 73 (HPV73), HPV type 74 (HPV74), HPV type 83 (HPV83), HPV type 84 (HPV84), HPV type 85 (HPV85), or a combination thereof.

In some embodiments, a vaccine comprises at least one RNA (e.g., mRNA) polynucleotide having an open reading frame encoding at least one (e.g., one, two, three, four, five, six, seven, or eight) of E1, E2, E4, E5, E6, E7, L1, and L2 protein obtained from HPV, or a combination thereof. In some embodiments, a vaccine comprises at least one RNA (e.g., mRNA) polynucleotide having an open reading frame encoding at least one (e.g., one, two, three, four, five, or six) polypeptide selected from E1, E2, E4, E5, E6, and E7 protein obtained from HPV, or a combination thereof. In some embodiments a vaccine comprises at least one RNA (e.g., mRNA) polynucleotide having an open reading frame encoding at least one polypeptide selected from E6 and E7 protein obtained from HPV, or a combination thereof. In some embodiments, a vaccine comprises at least one RNA (e.g., mRNA) polynucleotide having an open reading frame encoding a polypeptide selected from L1 or L2 protein obtained from HPV, or a combination thereof.

In some embodiments, the at least one RNA polynucleotide encodes an antigenic polypeptide that structurally modifies an infected cell.

In some embodiments, the at least one RNA polynucleotide encodes an antigenic polypeptide that forms part or all of the HPV viral capsid.

In some embodiments, the at least one RNA polynucleotide encodes an antigenic polypeptide that is capable of self-assembling into virus-like particles.

In some embodiments, the at least one RNA polynucleotide encodes an antigenic polypeptide that is responsible for binding of the HPV to a cell being infected.

Some embodiments of the disclosure concern methods of treating and/or preventing HPV infection in humans, wherein one or more of the compositions described herein, which contain one or more immunomodulatory therapeutic nucleic acids encoding an immune potentiator construct and at least one HPV polypeptide or an immunogenic fragment thereof, that have been shown or are predicted by one skilled in the art to produce an immune response, is provided to a subject in need thereof (e.g. a person that is infected with or who is at risk of infection by HPV).

In some embodiments, the disclosure concerns methods of treating and/or preventing cancer resulting from and/or causally associated with HPV infection. In some embodiments, the disclosure provides a method to reduce the HPV infection or at least one symptom resulting from HPV infection. In some embodiments, the disclosure provides a method to reduce the risk of cervical, penile, vaginal, vulval, anal or oropharyngeal cancer in a subject. In each of these methods, one or more of the compositions described herein, which contain one or more immunomodulatory therapeutic nucleic acids encoding an immune potentiator construct and at least one HPV polypeptide or an immunogenic fragment thereof, that have been shown or are predicted by one skilled in the art to produce an immune response, is provided to a subject in need thereof (e.g. a person that is infected with or who is at risk of infection by HPV).

Optionally, a subject in need of a medicament that prevents and/or treats HPV infection is provided a medicament comprising an immune potentiator construct and one or more of the immunomodulatory therapeutic nucleic acids encoding at least one HPV polypeptide or an immunogenic fragment thereof, to produce an immune response directed toward HPV and/or to the subject's cells that are infected with HPV. In some embodiments, the immune response results in a reduction in HPV viral titer. In some embodiments, the immune response results in the production of neutralizing anti-HPV antibodies. In some embodiments, the immune response results in a cytotoxic T-cell response directed at HPV infected cells.

### B. Hepatitis B Virus (HBV)

In another embodiment, the oncoviral antigen is from the hepatitis B virus (HBV). The Hepatitis B Virus (HBV) is a double-stranded DNA virus belonging to the Hepadnaviridae family. Upon infection of humans, HBV causes the disease hepatitis B. In addition to causing hepatitis, infection with HBV can lead to the development of cirrhosis and hepatocellular carcinoma. Accordingly, in another aspect, an immune potentiator construct can be used to enhance an immune response against one or more Hepatitis B Virus (HBV) antigens of interest. For example, an antigen(s) of interest from HBV can be encoded by a chemically modified mRNA (mmRNA), provided on the same mmRNA as the immune potentiator construct or provided on a different construct mmRNA construct as the immune potentiator. The immune potentiator and HBV antigen mmRNAs can be formulated (or coformulated) and administered (simultaneously or sequentially) to a subject in need thereof to stimulate an immune response against the HBV antigen in the subject.

The HBV genome encodes four overlapping open reading frames (i.e. genes) demarcated by the letters S, C, P, and X (Ganem et al., (2001) Fields Virology 4th ed.; Hollinger et al., (2001) Fields Virology 4^{th} ed.). The S gene encodes the viral surface envelope proteins, the HBsAg, and can be structurally and functionally divided into the pre-S1, pre-S2, and S regions. There are three forms of HBSAG, small(S), middle (M), and large (L). The core or C gene has the precore and core regions. Multiple in-frame translation initiation codons are a feature of the S and C genes, which give rise to related but functionally distinct proteins. The C gene encodes either the viral nucleocapsid HBcAg or hepatitis B e antigen (HBeAg) depending on whether translation is initiated from the core or precore regions, respectively. The core protein self-assembles into a capsid-like structure. The precore ORF encodes a signal peptide that directs the translation product to the endoplasmic reticulum of the infected cell, where the protein is further processed to form the secreted HBeAg. The function of HBeAg is largely uncharacterized, although it has been implicated in immune tolerance, whose function is to promote persistent infection (Milich and Liang (2003) Hepatology 38:1075-1086. The polymerase (pol) is a large protein of approximately 800 amino acids and is encoded by the P ORF. Pol is functionally divided into three domains: the terminal protein domain, which is involved in encapsidation and initiation of minus-strand synthesis; the reverse transcriptase (RT) domain, which catalyzes genome synthesis; and the ribonuclease H domain, which degrades pregenomic RNA and facilitates replication. The HBV X ORF encodes a 16.5-kd protein (HBxAg) with multiple functions, including signal transduction, transcriptional activation, DNA repair, and inhibition of protein degradation (Cross et al., (1993) Proc Natl Acad Sci USA 90:8078-8082; Bouchard and Schneider (2004) J Virol 78:12725-12734). The mechanism of this activity and the biologic function of HBxAg in the viral life-cycle remain largely unknown. However, it is well-established that HBxAg is necessary for productive HBV infection in vivo and may contribute to the oncogenic potential of HBV (Liang (2009) Hepatology 49(Suppl S5):S13-S21).

Despite the availability of an effective prophylactic vaccine, over 240 million people remain chronically infected with HBV and more than 500,000 people die each year from the liver diseases that result from chronic infection (World Health Organization (2015) Hepatitis B Fact Sheet FS204). The currently available therapeutic options for HBV infection include nucleos(t)ide analogues and alpha interferon (IFN-α). However, these treatments have several limitations. Nucleos(t)ide analogues effectively suppress virus replication but do not eliminate the infection. Once treatment with nucleos(t)ide analogues is stopped, the virus rapidly rebounds in the infected person. Furthermore, long-term treatment with antivirals can result in the generation of drug-resistant mutant viruses. In contrast to nucleos(t)ide analogues, IFN-α, which has both antiviral and immunomodulatory activities, can produce more durable results in some patients. However, IFN-α treatment is often associated with a high incidence of side effects, which makes it a suboptimal treatment option. Therefore, the design of new effective treatments for HBV-associated infection and disease is essential (Reynolds et al., (2015) J Virol 89 (20): 10407-10415).

HBV infection and its treatment are typically monitored by the detection of viral antigens and/or antibodies against the antigens. Upon infection with HBV, the first detectable antigen is the hepatitis B surface antigen (HBsAg), followed by the hepatitis B “e” antigen (HBeAg). Clearance of the virus is indicated by the appearance of IgG antibodies in the serum against HBsAg and/or against the core antigen (HBcAg), also known as seroconversion. Numerous studies indicate that viral replication, the level of viremia and progression to the chronic state in HBV-infected individuals are influenced directly and indirectly by HBV-specific cellular immunity mediated by CD4+ helper (TR) and CD8+ cytotoxic T lymphocytes (CTLs). Patients progressing to chronic disease tend to have absent, weaker, or narrowly focused HBV-specific T cell responses as compared to patients who clear acute infection (see, e.g., Chisari, 1997, J Clin Invest 99:1472-1477; Maini et al, 1999, Gastroenterology 117:1386-1396; Rehermann et al, 2005, Nat Rev Immunol 2005; 5:215-229; Thimme et al, 2001, J Virol 75:3984-3987; Urbani et al, 2002, J Virol 76:12423-12434; Wieland and Chisari, 2005, J Virol 79:9369-9380; Webster et al, 2000, Hepatology 32:1117-1124; Penna et al, 1996, J Clin Invest 98:1185-1194; Sprengers et al, 2006, J Hepatol 2006; 45:182-189.)

In some embodiments, a RNA (e.g., mRNA) vaccine (e.g., comprising an immune potentiator construct and an HBV antigen construct, on the same or different mRNAs) comprises at least one RNA (e.g., mRNA) polynucleotide having an open reading frame encoding at least one HBV antigenic polypeptide or an immunogenic fragment thereof (e.g., an immunogenic fragment capable of inducing an immune response to HBV). In some embodiments, at least one HBV antigenic polypeptide is selected from HBsAg (S, M or L), HBcAg, HBeAg, HBxAg, Pol, and combinations thereof.

Based on intergroup divergence across sequenced genomes, HBV has been classified phylogenetically into 9 genotypes, A-I, with a putative 10th genotype, J, isolated from a single individual. The HBV genotypes are further classified into at least 35 subgenotypes. Genotype differences impact disease severity, disease course and likelihood of complications, response to treatment and possibly response to vaccination (Kramvis et al., (2005), Vaccine 23(19):2409-2423; Magnius and Norder, (1995), Intervirology 38(1-2):24-34).

HBV genotype A is further classified into subgenotypes A1, A2, A4, and the quasi-subgenotype A3, the latter group of sequences does not meet the criteria for a subgenotype classification. HBV genotype B is further classified into 6 subgenotypes B1, B2, B4-B6, and quasi-subgenotype B3. HBV genotype C, the oldest HBV genotype, is further classified into 16 subgenotypes C1-C16, reflecting the long duration of endemicity in the human population. HBV genotype D is further classified into 6 subgenotypes D1-D6. HBV genotype F is further classified into 4 subgenotypes F1-F4. Genotype I is further classified into 2 subgenotypes I1 and 12. Furthermore, HBV has been classified by serology into 4 major serotypes adr, adw, ayr, and ayw based on antigenic epitopes present on HBV's envelope proteins (Kramvis (2014) Intervirology 57:141-150).

In some embodiments, the at least one HBV antigenic polypeptide is from HBV genotype A (e.g., any of subgenotypes A1-A4), HBV genotype B (e.g, any of subgenotypes B1-B6), HBV genotype C (e.g., any of subgenotypes C1-C16), HBV genotype D (e.g., any of subgenotypes D1-D6), HBV genotype E, HBV genotype F (e.g, any of subgenotypes F1-F4), HBV genotype G or HBV genotype I (e.g., any of subgenotypes I1-12).

Some embodiments of the disclosure concern methods of treating and/or preventing HBV infection in humans, wherein one or more of the compositions described herein, which contain one or more immunomodulatory therapeutic nucleic acids encoding an immune potentiator construct and at least one HBV polypeptide or an immunogenic fragment thereof, that have been shown or are predicted by one skilled in the art to produce an immune response, is provided to a subject in need thereof (e.g. a person that is infected with or who is at risk of infection by HBV).

In some embodiments, the disclosure concerns methods of treating and/or preventing cancer resulting from and/or causally associated with HBV infection. In some embodiments, the disclosure provides a method to reduce the HBV infection or at least one symptom resulting from HBV infection. In some embodiments, the disclosure provides a method to reduce liver damage in a subject. In each of these methods, one or more of the compositions described herein, which contain one or more immunomodulatory therapeutic nucleic acids encoding an immune potentiator construct and at least one HBV polypeptide or an immunogenic fragment thereof, that have been shown or are predicted by one skilled in the art to produce an immune response, is provided to a subject in need thereof (e.g. a person that is infected with or who is at risk of infection by HBV).

Optionally, a subject in need of a medicament that prevents and/or treats HBV infection is provided a medicament comprising an immune potentiator construct and one or more of the immunomodulatory therapeutic nucleic acids encoding at least one HBV polypeptide or an immunogenic fragment thereof, to produce an immune response directed toward HBV and/or to the subject's cells that are infected with HBV. In some embodiments, the immune response results in a reduction in HBV viral titer. In some embodiments, the immune response results in the production of neutralizing anti-HBV antibodies. In some embodiments, the immune response results in a cytotoxic T-cell response directed at HBV infected cells.

In some embodiments, an immunomodulatory therapeutic nucleic acid (e.g., messenger RNA, mRNA) comprises at least one (e.g., mRNA) polynucleotide having an open reading frame encoding at least one HBV antigenic polypeptide or an immunogenic fragment thereof (e.g., an immunogenic fragment capable of inducing an immune response to HBV). In some embodiments, the at least one antigenic polypeptide or immunogenic fragment thereof is selected from HBsAg, HBcAg, HBeAg, HBxAg, or Pol.

In some embodiments, the at least one antigenic polypeptide or immunogenic fragment thereof is selected from provisional and/or confirmed HBV genotypes and/or subgenotypes. In some embodiments, the at least one antigenic polypeptide or immunogenic fragment thereof is selected from provisional or unassigned HBV genotypes or subgenotypes.

In some embodiments, the at least one RNA polynucleotide encodes an antigenic polypeptide that structurally modifies an infected cell.

In some embodiments, the at least one RNA polynucleotide encodes an antigenic polypeptide that forms part or all of the HBV viral capsid.

In some embodiments, the at least one RNA polynucleotide encodes an antigenic polypeptide that is capable of self-assembling into virus-like particles.

In some embodiments, the at least one RNA polynucleotide encodes an antigenic polypeptide that is responsible for binding of the HBV virus to a cell being infected.

### C. Hepatitis C Virus (HCV)

In another embodiment, the oncoviral antigen is from the hepatitis C virus (HCV). The hepatitis C virus (HCV) is a small, enveloped, positive-sense single-stranded RNA virus that causes hepatitis C, a viral infectious disease that primarily affects the liver. Accordingly, in another aspect, an immune potentiator construct can be used to enhance an immune response against one or more Hepatitis C Virus (HCV) antigens of interest. For example, an antigen(s) of interest from HCV can be encoded by a chemically modified mRNA (mmRNA), provided on the same mmRNA as the immune potentiator construct or provided on a different construct mmRNA construct as the immune potentiator. The immune potentiator and HCV antigen mmRNAs can be formulated (or coformulated) and administered (simultaneously or sequentially) to a subject in need thereof to stimulate an immune response against the HCV antigen in the subject.

The RNA genome of HCV encodes a large polyprotein of 3010 amino acids that is co- an post-translationally processed by cellular and virally encoded proteases and peptidases to produce the mature structural and non-structural (NS) proteins. The HCV structural proteins include Core (alternatively C or p22), and two envelope glycoproteins E1 and E2 (alternatively gp35 and gp70, respectively). The non-structural (NS) proteins include NS1 (alternatively p7), NS2 (alternatively p23), NS3 (alternatively p70), NS4A (alternatively p8), NS4B (alternatively p27), NS5A (alternatively p56/58), and NS5B (alternatively p68) (Ashfaq et al., (2011) Virol J 8:161).

On the basis of phylogenetic and sequence analyses of whole viral genomes, HCV variants are currently classified into 7 separate genotypes and more than 80 confirmed and provisional subtypes (Smith et al., (2014) Hepatology 59(1):318-327). The International Committee for Taxonomy of Viruses (ICTV) maintains and regularly updates tables of reference isolates, confirmed and provisional subtypes, unassigned HCV isolates, accession numbers, and annotated alignments (http://talk.ictvonline.org/links/hcv/hcv-classification.htm). HCV subtypes 1a, 1b, 2a, and 3a are considered “epidemic subtypes”, are globally distributed, and account for a large proportion of HCV infections in high-income countries. These subtypes are thought to have spread rapidly in the years prior to the discovery of HCV transmission by way of infected blood, blood products, intravenous drug use, and other routes (Smith et al., (2005) J Gen Virol 78(Pt2):321-328; Pybus et al., (2005) Infect Genet Evol 5:131-139; Magiorkinis et al., (2009) PLOS Med 6:e1000198). Other HCV subtypes are considered “endemic” strains, are comparatively rare, and have circulated for long periods of time in more restricted regions. Endemic strains from genotypes 1 and 2 are primarily localized to West Africa, 3 in south Asia, 4 in Central Africa and the Middle East, 5 in Southern Africa, and 6 in South East Asia (Simmonds (2001) J Gen Virol 82:693:712; Pybus et al., (2009) J Virol 83:1071-1082). To date, only one genotype 7 infection has been reported (Murphy et al., (2007) J Clin Microbiol 45:1102-1112).

HCV naturally infects only humans, although chimpanzees have been shown to be susceptible to experimental infection (Pfaender et al., (2014) Emerg Microbes Infect 3:e21). Chronic viral infection by HCV is a leading cause of cirrhosis, liver disease, portal hypertension, deteriorating liver function, and cancer (e.g. hepatocellular carcinoma, HCC) (Webster et al., (2015) Lancet 385(9973):1124-1135). Over 160-170 million people worldwide are estimated to have hepatitis C, which ultimately causes approximately 350,000 deaths per year (Zaltron et al., (2012) BMC Infect Dis 12 (Suppl 2): S2; Lavanchy (2011) Clin Microbiol Infect 17:107-115). Globally, approximately one quarter of all cirrhosis and HCC cases are attributed to HCV infection. However, in regions of high endemicity, HCV usually accounts for greater than 50% of HCC and cirrhosis cases (Perz et al., (2006) J Hepatol 45 (4): 529-538). Chronically infected people have a decreased quality of life compared to the general population (Bezemer et al., (2012) BMC Gastroenterol 12:11).

Blood and blood product transfusion was previously the major route of HCV transmission prior to the implementation of universal screening (Zou et al., (2010) Transfusion 50(7):1495-1504). Percutaneous transmission via intravenous drug use is now the major route of transmission in developed countries (Cornberg et al., (2011) Liver Int 31(Suppl 2):30-60; Nelson et al., (2011) Lancet 378(9791:571-583). Social services such as needle and syringe exchange programmes (NSPs) and opiate substitution therapy (OST) can effectively reduce HCV transmission among people who inject drugs (PWID), but these approaches may be insufficient for reducing HCV prevalence to low levels (Turner et al., (2011) Addiction 106(11)1978-1988; Vikermann et al., (2012) Addiction 107 (11): 1984-1995). Very recently, highly effective direct-acting antiviral therapies (DAAs) have been developed and used to treat HCV infections (e.g. boceprevir, telaprevir, simeprevir, sofosbuvir, ledipasvir, ombitasvir, paritaprevir, ritonavir, dasabuvir, daclatasvir, elbasvir, grazoprevir, velpatasvir). Since DAAs can lead to a sustained virologic response (SVR, alternatively “viral cure”) in many patients, these drugs demonstrate potential for a treatment-as-prevention approach to decrease HCV prevalence (Smith-Palmer et al., (2015) BMC Infect Dis 15:19). However, the high financial cost and challenges of payer reimbursement decisions regarding these treatments currently restricts their widespread use (Martin et al., (2011) J Hepatol 54(6):1137-1144; Martin et al., (2012) Hepatology 55 (1): 49-57; Brennan and Shrank (2014) JAMA 312(6):593-594).

HCV vaccination is an alternative treatment and/or prevention strategy to decrease HCV prevalence. Early HCV vaccine studies in experimentally-infected chimpanzees found that a subunit vaccine composed of viral envelope glycoproteins E1 (gp35) and E2 (gp72) elicited a high efficacy humoral response that effectively controlled and facilitated clearance of the homologous HCV genotype 1a virus (Choo et al., (1994) Proc Nat Acad Sci USA 91 (4): 1294-1298). Phase I studies conducted in humans demonstrated that a vaccine comprising glycoproteins E1 and E2 elicited broadly reactive neutralizing antibodies (Law et al., (2013) PLOS ONE 8(3):59776). An alternative vaccination approach designed to generate T-cell responses against HCV has also been tested in human phase 1 studies and was shown to be highly immunogenic (Barnes et al., (2012) Sci Trans Med 4 (115): 115ra1). These studies have demonstrated that both humoral, antibody-mediated immune responses and/or adaptive, T-cell-mediated responses are promising approaches for the development of a prophylactic and/or therapeutic HCV vaccine.

In some embodiments, a RNA (e.g., mRNA) vaccine (e.g., comprising an immune potentiator construct and an HCV antigen construct, on the same or different mRNAs) comprises at least one RNA (e.g., mRNA) polynucleotide having an open reading frame encoding at least one HCV antigenic polypeptide or an immunogenic fragment thereof (e.g., an immunogenic fragment capable of inducing an immune response to HCV). In some embodiments, at least one HCV antigenic polypeptide is selected from Core (C, p22), E1 (gp35), E2 (gp70), NS1 (p7), NS2 (p23), NS3 (p70), NS4A (p8), NS4B (p27), NS5A (p56/58), NS5B (p68), and combinations thereof.

Some embodiments of the disclosure concern methods of treating and/or preventing HCV infection in humans, wherein one or more of the compositions described herein, which contain one or more immunomodulatory therapeutic nucleic acids encoding an immune potentiator construct and at least one HCV polypeptide or an immunogenic fragment thereof, that have been shown or are predicted by one skilled in the art to produce an immune response, is provided to a subject in need thereof (e.g. a person that is infected with or who is at risk of infection by HCV). Optionally, a subject in need of a medicament that prevents and/or treats HCV infection is provided a medicament comprising one or more of the immunomodulatory therapeutic nucleic acids encoding an immune potentiator construct and at least one HCV polypeptide or an immunogenic fragment thereof, to produce an immune response directed toward HCV and/or to the subject's cells that are infected with HCV. In some embodiments, the immune response results in a reduction in HCV viral titer and/or the establishment of a sustained virologic response. In some embodiments, the immune response results in the production of neutralizing anti-HCV antibodies. In some embodiments, the immune response results in a cytotoxic T-cell response directed at HCV infected cells.

In some embodiments, an immunomodulatory therapeutic nucleic acid (e.g., messenger RNA, mRNA) comprises at least one (e.g., mRNA) polynucleotide having an open reading frame encoding at least one HCV antigenic polypeptide or an immunogenic fragment thereof (e.g., an immunogenic fragment capable of inducing an immune response to HCV). In some embodiments, the at least one antigenic polypeptide or immunogenic fragment thereof is selected from Core (C, p22), E1 (gp35), E2 (gp70), NS1 (p7), NS2 (p23), NS3 (p70), NS4A (p8), NS4B (p27), NS5A (p56/58), NS5B (p68), and combinations thereof.

In some embodiments, the at least one antigenic polypeptide or immunogenic fragment thereof is selected from confirmed HCV genotypes and/or subtypes 1, 1a, 1b, 1c, 1d, 1e, 1g, 1h, 1i, 1j, 1k, 1l, 1m, 1n, 2, 2a, 2b, 2c, 2d, 2e, 2f, 2i, 2j, 2k, 2l, 2m, 2q, 2r, 2t, 2u, 3, 3a, 3b, 3d, 3e, 3g, 3h, 3i, 3k, 4, 4a, 4b, 4c, 4d, 4f, 4g, 4k, 4l, 4m, 4n, 40, 4p, 4q, 4r, 4s, 4t, 4v, 4w, 5, 5a, 6, 6a, 6b, 6c, 6d, 6e, 6f, 6g, 6h, 6i, 6j, 6k, 61, 6m, 6n, 60, 6p, 6q, 6r, 6s, 6t, 6u, 6v, 6w, 6xa, 6xb, 6xc, 6xd, 6xe, 7, or 7a. In some embodiments, the at least one antigenic polypeptide or immunogenic fragment thereof is selected from provisional HCV genotypes and/or subtypes 1f, 2g, 2h, 2n, 20, 2p, 2s, 3c, 3f, 4e, 4h, 4i, or 4j. In some embodiments, the at least one antigenic polypeptide or immunogenic fragment thereof is selected from provisional or unassigned HCV isolates.

In some embodiments, the at least one RNA polynucleotide encodes an antigenic polypeptide that structurally modifies an infected cell.

In some embodiments, the at least one RNA polynucleotide encodes an antigenic polypeptide that forms part or all of the HCV viral capsid.

In some embodiments, the at least one RNA polynucleotide encodes an antigenic polypeptide that is capable of self-assembling into virus-like particles.

In some embodiments, the at least one RNA polynucleotide encodes an antigenic polypeptide that is responsible for binding of the HCV to a cell being infected.

### D. Epstein-Barr Virus (EBV)

In another embodiment, the oncoviral antigen is from the Epstein-Barr Virus (EBV). The Epstein-Barr virus (EBV), alternatively human herpesvirus 4 (HHV-4), is the etiological agent of infectious mononucleosis and is associated with a large number of benign and malignant diseases, including several human cancers (e.g. Hodgkin's lymphoma, non-Hodgkin's lymphoma, Burkitt's lymphoma, breast cancer, hepatocellular carcinomas, gastric/stomach carcinoma, post-transplant lymphoproliferative disease (PTLD), central nervous system lymphoma (CNS), nasopharyngeal carcinoma, multiple sclerosis, EBV-associated lymphomas, oral hairy leukoplakia, diffuse large B-cell lymphoma, AIDS-related lymphoma) (Jha et al., (2016) Front Microbiol 7 (1602) and references therein). EBV is an extremely prevalent virus infecting >95% of the world's adult population (Cohen (2000) N Engl J Med 343:481-492). Accordingly, in another aspect, an immune potentiator construct can be used to enhance an immune response against one or more Epstein-Barr Virus (EBV) antigens of interest. For example, an antigen(s) of interest from EBV can be encoded by a chemically modified mRNA (mmRNA), provided on the same mmRNA as the immune potentiator construct or provided on a different construct mmRNA construct as the immune potentiator. The immune potentiator and EBV antigen mmRNAs can be formulated (or coformulated) and administered (simultaneously or sequentially) to a subject in need thereof to stimulate an immune response against the EBV antigen in the subject.

The EBV genome is a linear double-stranded DNA (dsDNA) molecule, approximately 172 kb in length. The EBV genome has the coding potential for approximately 80 viral proteins, many whose function remains uncharacterized. Characterized EBV genes, including their corresponding gene products and proposed function, if known, include BKRF1 (EBNA1) [plasmid maintenance, DNA replication, transcriptional regulation], BYRF1 (EBNA2) [trans-activation], BLRF3/BERF1 (EBNA3A, alternatively EBNA3) [transcriptional regulation], BERF2a/b (EBNA3B, alternatively EBNA4), BERF3/4 (EBNA3C, alternatively EBNA6) [transcriptional regulation], BWRF1 (EBNA-LP, alternatively EBNA5) [trans-activation], BNLF1 (LMP1) [B-cell survival, anti-apoptosis], BNRF1 (LMP2A/B, alternatively TP1/2) [maintenance of latency], BARF0 (A73, RPMS1), EBER1/2 (small RNAs) [regulation of innate immunity], BZLF1 (ZEBRA/Zta/EB1) [trans-activation, initiation of lytic cycle], BRLF1 [trans-activation, initiation of lytic cycle], BILF4 [trans-activation, initiation of lytic cycle], BMRF1 [trans-activation], BALF2 [DNA binding], BALF5 [DNA polymerase], BORF2 [ribonucleotide reductase subunit], BARF1 [ribonucleotide reductase subunit], BXLF1 [thymidine kinase], BGLF5 [alkaline exonuclease], BSLF1 [primase], BBLF4 [helicase], BKRF3 [uracil DNA glycosylase], BLLF1 (gp350/220) [major envelope glycoprotein], BXLF2 (gp85, alternatively gH) [virus-host envelope fusion], BKRF2 (gp25, alternatively gL) [virus-host envelope fusion], BZLF2 (gp42) [virus-host envelope fusion, binds MHC class II], BALF4 (gp110, alternatively gB), BDLF3 (gp100-150), BILF2 (gp55-78), BCRF1 [viral interleukin-10], and BHRF1 [viral bcl-2 analogue] (Liebowitz and Kieff (1993) Epstein-Barr virus. In: The Human Herpesvirus. Roizman B, Whitley R J, Lopez C, editors, New York, pp. 107-172; Li et al., (1995) J Virol 69:3987-3994; Nolan and Morgan (1995) J Gen Virol 76:1381-1392; Thompson and Kurzrock (2004) Clin Cancer Res 10:803-821; Young and Murray (2003) Oncogene 22:5108-5121).

In some embodiments, a RNA (e.g., mRNA) vaccine (e.g., comprising an immune potentiator construct and an EBV antigen construct, on the same or different mRNAs) comprises at least one RNA (e.g., mRNA) polynucleotide having an open reading frame encoding at least one EBV antigenic polypeptide or an immunogenic fragment thereof (e.g., an immunogenic fragment capable of inducing an immune response to EBV). Any of the afore-mentioned EBV proteins can be used as the antigenic EBV polypeptide. Immunogenic EBV proteins and their epitopes have been described in the art (e.g., Rajcani J. et al. (2014) Recent Pat. Antiinfect. Drug Discover. 9:62-76). In certain embodiments, the antigenic EBV polypeptide is selected from the group consisting of BLLF1 (gp350/220), BZLF1/Zta, EBNA2, EBNA3, EBNA6, LMP1, LMP2A, and combinations thereof.

Two major EBV types are known to infect humans: EBV-1 and EBV-2 (alternatively known as types A and B or as the B95-8 strain and AG876 strain, respectively). The two EBV types differ in the sequence of genes that encode the EBV nuclear antigens EBNA-2, EBNA-3A/3, EBNA-3B/4, and EBNA-3C/6 (Sample et al., (1990) J Virol 64:4084-4092; Dambaugh et al., (1984) Proc Natl Acad Sci USA 81:7632-7636). Within the two major EBV types, extensive strain diversity is observed in EBVs isolated from clinical samples, which may play a role in disease type and severity. The first complete EBV genome sequence, B95-8, was published in 1984 (Baer et al., (1984) Nature 310:207-211). The genome sequences of 22 additional EBVs have been reported (AG876, GD1, GD2, HKNPC1, Akata, Mutu, C666-1, M81, Raji, K4123-Mi, and K4413-Mi), as well as eight EBV sequences derived from nasopharyngeal carcinoma clinical samples and three EBV genomes derived from the 1000 Genomes project (Tsai et al., (2013) Cell Rep 5:458-470; Dolan et al., (2006) Virology 350-164-170; Palser et al., (2015) J Virol 89 (10): 5222-5237 and references therein). A recent report analyzed the genomic sequences of 71 new EBV genomes, including the first EBV genome sequenced directly from saliva. These new EBV genomic sequences were analyzed in combination with the 12 previously published strains. This analysis revealed that the established gene map of the EBV genome (NC_007605) is representative of EBV isolates from different geographic locations and from different types of infection. The well-established EBV type 1 and type 2 classification was reexamined in this study and was found to remain the major form of variation, mostly accounted for by variation in EBNA2 and EBNA3A, —B, and —C(Palser et al., (2015) J Virol 89(10):5222-5237).

In some embodiments, the at least one EBV antigenic polypeptide is from EBV-1 or EBV-2.

Some embodiments of the disclosure concern methods of treating and/or preventing EBV infection in humans, wherein one or more of the compositions described herein, which contain one or more immunomodulatory therapeutic nucleic acids encoding an immune potentiator construct and at least one EBV polypeptide or an immunogenic fragment thereof, that have been shown or are predicted by one skilled in the art to produce an immune response, is provided to a subject in need thereof (e.g. a person that is infected with or who is at risk of infection by EBV). Optionally, a subject in need of a medicament that prevents and/or treats EBV infection is provided a medicament comprising one or more of the immunomodulatory therapeutic nucleic acids encoding an immune potentiator construct and at least one EBV polypeptide or an immunogenic fragment thereof, to produce an immune response directed toward EBV and/or to the subject's cells that are infected with EBV. In some embodiments, the immune response results in a reduction in EBV viral titer and/or the establishment of a sustained virologic response. In some embodiments, the immune response results in the production of neutralizing anti-EBV antibodies. In some embodiments, the immune response results in a cytotoxic T-cell response directed at EBV infected cells.

In some embodiments, the at least one RNA polynucleotide encodes an antigenic polypeptide that structurally modifies an infected cell.

In some embodiments, the at least one RNA polynucleotide encodes an antigenic polypeptide that forms part or all of the EBV viral capsid.

In some embodiments, the at least one RNA polynucleotide encodes an antigenic polypeptide that is capable of self-assembling into virus-like particles.

In some embodiments, the at least one RNA polynucleotide encodes an antigenic polypeptide that is responsible for binding of the EBV to a cell being infected.

### E. Human T-Cell Lymphotropic Virus Type 1 (HTLV-1)

In another embodiment, the oncoviral antigen is from Human T-cell lymphotropic virus type 1 (HTLV-1). The human T-cell lymphotropic virus type 1 (HTLV-1, alternatively human T-lymphotropic virus or human T-cell leukemia-lymphoma virus) is a retrovirus that is capable of establishing a persistent infection in humans. HTLV-1 infects an estimated 10-20 million people worldwide and while infection is asymptomatic in most people, 3%-5% of infected individuals develop a highly malignant and therapeutically intractable adult T-cell leukemia/lymphoma (ATL) (Gessain et al., (2012) Front Microbiol 3:388; Taylor et al., (2005) Oncogene 24:6047-6057). HTLV infection is also causatively associated with several inflammatory and immune-mediated disorders, most notably HTLV-associated myleopathy/tropical spastic paraparesis (HAM/TSP). Approximately 0.25%-3.8% of HTLV-1-infected people develop HAM/TSP (Yamano and Sato (2012) Front Microbiol 3:389). Human transmission of HTLV-1 requires transfer of virus-infected cells via breast-feeding, sexual intercourse, transfusion of cell-containing blood components, and sharing of needles and/or syringes (e.g. intravenous drug use). Accordingly, in another aspect, an immune potentiator construct can be used to enhance an immune response against one or more Human T-cell lymphotropic virus type 1 (HTLV-1) antigens of interest. For example, an antigen(s) of interest from HTLV-1 can be encoded by a chemically modified mRNA (mmRNA), provided on the same mmRNA as the immune potentiator construct or provided on a different construct mmRNA construct as the immune potentiator. The immune potentiator and HTLV-1 antigen mmRNAs can be formulated (or coformulated) and administered (simultaneously or sequentially) to a subject in need thereof to stimulate an immune response against the HTLV-1 antigen in the subject.

HTLV-1 is a complex retrovirus; in addition to the standard repertoire of structural proteins and enzymes shared by all retroviridae (gag, pol, pro and env), the 3′ region of the HTLV-1 genome (alternatively called the pX region) encodes accessory genes tax, rex, p12, p21, p13, p30 and HBZ. Tax and HBZ are indispensable in the oncogenic process of ATL (Giam and Semmes (2016) Viruses 8 (6): 161). Similar to other retroviruses, after transmission, viral reverse transcriptase generates proviral DNA from genomic viral RNA. The provirus is integrated into the host genome by viral integrase. Afterwards, HTLV-1 infection is thought to spread only through dividing cells, with minimal particle production. The quantification of provirus reflects the number of HTLV-1-infected cells, which defines the proviral load (Concalves et al., (2010) Clin Microbiol Rev 23(3):577-589).

In some embodiments, a RNA (e.g., mRNA) vaccine (e.g., comprising an immune potentiator construct and an HTLV-1 antigen construct, on the same or different mRNAs) comprises at least one RNA (e.g., mRNA) polynucleotide having an open reading frame encoding at least one HTLV-1 antigenic polypeptide or an immunogenic fragment thereof (e.g., an immunogenic fragment capable of inducing an immune response to HTLV-1). In certain embodiments, the antigenic HTLV-1 polypeptide is selected from the group consisting of gag, pol, pro, env, tax, rex, p12, p21, p13, p30, HBZ, and combinations thereof.

Some embodiments of the disclosure concern methods of treating and/or preventing HTLV-1 infection in humans, wherein one or more of the compositions described herein, which contain one or more immunomodulatory therapeutic nucleic acids encoding an immune potentiator construct and at least one HTLV-1 polypeptide or an immunogenic fragment thereof, that have been shown or are predicted by one skilled in the art to produce an immune response, is provided to a subject in need thereof (e.g. a person that is infected with or who is at risk of infection by HTLV-1). Optionally, a subject in need of a medicament that prevents and/or treats HTLV-1 infection is provided a medicament comprising one or more of the immunomodulatory therapeutic nucleic acids encoding an immune potentiator construct and at least one HTLV-1 polypeptide or an immunogenic fragment thereof, to produce an immune response directed toward HTLV-1 and/or to the subject's cells that are infected with HTLV-1. In some embodiments, the immune response results in a reduction in HTLV-1 viral titer and/or the establishment of a sustained virologic response. In some embodiments, the immune response results in the production of neutralizing anti-HTLV-1 antibodies. In some embodiments, the immune response results in a cytotoxic T-cell response directed at HTLV-1 infected cells.

In some embodiments, the at least one RNA polynucleotide encodes an antigenic polypeptide that structurally modifies an infected cell.

In some embodiments, the at least one RNA polynucleotide encodes an antigenic polypeptide that forms part or all of the HTLV-1 viral capsid.

In some embodiments, the at least one RNA polynucleotide encodes an antigenic polypeptide that is capable of self-assembling into virus-like particles.

In some embodiments, the at least one RNA polynucleotide encodes an antigenic polypeptide that is responsible for binding of the HTLV-1 to a cell being infected.

### F. Kaposi's Sarcoma Herpesvirus (KSHV)

In another embodiment, the oncoviral antigen is from Kaposi's Sarcoma Herpesvirus (KSHV). Kaposi's sarcoma-associated herpesvirus (KSHV; alternatively human herpesvirus-8, HHV-8) is a double-stranded DNA γ-herpesvirus belonging to the Rhadinovirus genus within the Herpesviridae family. KSHV is the etiologic agent of all forms of Kaposi's sarcoma, a cancer commonly occurring in AIDS patients, and is causally associated with primary effusion lymphoma (PEL; alternatively body cavity-based lymphoma, BCBL), some types of multicentric Castleman's disease (MCD; alternatively multicentric Castleman's disease (MCD)-linked plasmablastic lymphoma), and KSHV inflammatory cytokine syndrome (KICS) (Chang et al., (1994) Science 266:1865-1869; Dupin et al., (1999) Proc Natl Acad Sci USA 96:4546-4551; Boshoff & Weiss (2002) Nat Rev Cancer 2(5):373-382; Yarchoan et al., (2005) Nat Clin Pract Oncol 2 (8): 406-415; Cesarman et al., (1995) N Engl J Med 332(18):1186-1191; Staudt et al., (2004) Cancer Res 64(14):4790-4799; Soulier et al., (1995) Blood 86:1276-1280; Uldrick et al., (2010) Clin Infect Dis 51:350-358)). Accordingly, in another aspect, an immune potentiator construct can be used to enhance an immune response against one or more Kaposi's Sarcoma Herpesvirus (KSHV) antigens of interest. For example, an antigen(s) of interest from KSHV can be encoded by a chemically modified mRNA (mmRNA), provided on the same mmRNA as the immune potentiator construct or provided on a different construct mmRNA construct as the immune potentiator. The immune potentiator and KSHV antigen mmRNAs can be formulated (or coformulated) and administered (simultaneously or sequentially) to a subject in need thereof to stimulate an immune response against the KSHV antigen in the subject.

The KSHV genome comprises an approximately 165 kb dsDNA molecule and exhibits a high degree of sequence identity across the viral strains and isolates. Two major gene regions, K1/VIP (a variable immunoreceptor tyrosine-based activation motif protein, encoded by the 5′ terminus of the KSHV genome) and K15/LAMP (a latency-associated membrane protein, encoded by the 3′ terminus of the KSHV genome), located at the terminal ends of the viral genome, are highly variable compared to the central genomic region (Zong et al., (1999) J Virol 73:4156-4170; Poole et al., (1999) 73:6646-6660).

The sequence variability of the K1 gene has led to the determination of five major KSHV subtypes (A, B, C, D, and E), displaying up to 35% variability at the amino acid level across the viral strains. The sequence analysis of the K15 gene has led to the additional categorization of KSHV sequences, with variants designated as P, M, or N alleles, differing by up to 70% at the amino acid level (Hayward & Zong (2007) Curr Top Microbiol Immunol 312:1-42). Nine other viral genomic loci (approximately 5.6% of the genome) contain additional variability (T0.7/K12, K2, K3, ORF18/19, ORF26, K8, ORF73), as well as two loci within the ORF75 gene regions, within the central, more conserved region of the KSHV genome. Based on the K1/K15 variability, strain classification, and variability of nine ORFs, the known KSHV genomes are currently classified into 12 principal genotypes (Strahan et al., (2016) Viruses 8 (4): 92).

Essentially all cases of Kaposi's sarcoma carry KSHV and the continued presence of KSHV is required for tumorigenesis. The KSHV genome has the coding potential for approximately 90 proteins, many known to mediate viral replication, virus-host interactions, tumorigenesis, and immune suppression and evasion (Dittmer & Damania (2013) Curr Opin Virol 3:238-244), which can be considered potential therapeutic targets. Characterized KSHV genes, including their corresponding gene products and/or proposed function, if known, include ORFK1 (glycoprotein; KSHV ITAM signaling protein, KIS), ORF4 (Kaposi complement control protein, KCP; kaposica), ORF6 (ssDNA binding protein), ORF11 (dUTPase-related protein, DURP), ORFK2 (viral interleukin 6 homolog, vIL6), ORF70 (thymidylate synthase), ORFK4 (vCCL-2, vMIP-II, MIP-1b), ORFK4.1 (vCCL-3, vMIP-III, BCK), ORFK5 (modulator of immune response 2, MIR-2; E3 ubiquitin ligase), ORFK6 (vCCL-1, vMIP-I, MIP-1a), PAN (late gene expression), ORF16 (vBCL2, Bcl2 homolog), ORF17.5 (scaffold or assembly protein, SCAF), ORF18 (late gene regulation), ORF34 (binds to HIF-1a), ORF35 (required for efficient lytic virus reactivation), ORF36 (viral serine/threonine protein kinase), ORF37 (sox), ORF38 (tegument protein), ORF39 (glycoprotein M, gM), ORF45 (tegument protein; RSK activator), ORF46 (uracil deglycosylase), ORF47 (glycoprotein L, gL), ORF50 (RTA), ORFK8 (k-bZIP; replication associated protein, RAP), ORF57 (mRNA export/splicing), ORF58, ORF59 (processivity factor), ORF60 (ribonucleoprotein reductase), ORF61 (ribonucleoprotein reductase), ORFK12 (kaposin), ORF71 (vFLIP, ORFK13), ORF72 (vCyclin, vCYC), ORF73 (latency-associated nuclear antigen 1, LANA1), ORF8 (glycoprotein B, gB), ORF9 (DNA polymerase), ORF10 (regulator of interferon function), ORFK3 (modulator of immune response 1, MIR-1; E3 ubiquitin ligase), K5/6-AS, ORF17 (protease), ORF21 (thymidine kinase), ORF22 (glycoprotein H, gH), ORF23 (predicted glycoprotein), ORF24 (essential for replication), ORF25 (major capsid protein, MCP), ORF26 (minor capsid protein; triplex component 2, TRI-2), ORF27 (glycoprotein), ORF28 (BDLF3 EBV homolog), ORF29 (packaging protein), ORF30 (late gene regulation), ORF31 (nuclear and cytoplasmic), ORF32 (tegument protein), ORF33 (tegument protein), ORF40/41 (helicase-primase), ORF42 (tegument protein), ORF43 (portal capsid protein), ORF44 (helicase), ORF45.1, ORFK8.1A (glycoprotein, gp8.1A), ORFK8.1B (glycoprotein gp8.1B, ORF52 (tegument protein), ORF53 (glycoprotein N, gN), ORF54 (dUTPase/immunomodulatory), ORF55 (tegument protein), ORF56 (DNA replication), ORFK9 (vIRF1), ORFK10 (vIRF4), ORFK10.5 (vIRF3, LANA2), ORFK11 (vIRF2), ORF62 (triplex component 1, TRI-1), ORF65 (small capsid protein; small capsomer-interacting protein, SCIP), ORF66 (capsid), ORF67 (nuclear egress complex), ORF67.5, ORF68 (glycoprotein), ORF69 (BRLF2 nuclear egress), ORFK14 (vOX2), ORF74 (vGPCR), ORF75 (FGARAT), ORF2 (dihydrofolate reductase), ORF7 (virion protein, vGPCR), ORF48, ORF49 (activates JNK/p38), ORF63 (NLR homolog), ORF64 (deubiquitinase), ORFK15 (LMP1/2), and ORFK7 (viral inhibitor of apoptosis, vIAP).

In some embodiments, a RNA (e.g., mRNA) vaccine (e.g., comprising an immune potentiator construct and a KSHV antigen construct, on the same or different mRNAs) comprises at least one RNA (e.g., mRNA) polynucleotide having an open reading frame encoding at least one KSHV antigenic polypeptide or an immunogenic fragment thereof (e.g., an immunogenic fragment capable of inducing an immune response to KSHV). Any of the afore-mentioned KSHV proteins can be used as the antigenic KSHV polypeptide.

In some embodiments, the at least one KSHV antigenic polypeptide is from KSHV subtype A, KSHV subtype B, KSHV subtype C, KSHV subtype D or KSHV subtype E.

Some embodiments of the disclosure concern methods of treating and/or preventing KSHV infection in humans, wherein one or more of the compositions described herein, which contain one or more immunomodulatory therapeutic nucleic acids encoding an immune potentiator construct and at least one KSHV polypeptide or an immunogenic fragment thereof, that have been shown or are predicted by one skilled in the art to produce an immune response, is provided to a subject in need thereof (e.g. a person that is infected with or who is at risk of infection by KSHV). Optionally, a subject in need of a medicament that prevents and/or treats KSHV infection is provided a medicament comprising one or more of the immunomodulatory therapeutic nucleic acids encoding an immune potentiator construct and at least one KSHV polypeptide or an immunogenic fragment thereof, to produce an immune response directed toward KSHV and/or to the subject's cells that are infected with KSHV. In some embodiments, the immune response results in a reduction in KSHV viral titer and/or the establishment of a sustained virologic response. In some embodiments, the immune response results in the production of neutralizing anti-KSHV antibodies. In some embodiments, the immune response results in a cytotoxic T-cell response directed at KSHV infected cells.

In some embodiments, the at least one RNA polynucleotide encodes an antigenic polypeptide that structurally modifies an infected cell.

In some embodiments, the at least one RNA polynucleotide encodes an antigenic polypeptide that forms part or all of the KSHV viral capsid.

In some embodiments, the at least one RNA polynucleotide encodes an antigenic polypeptide that is capable of self-assembling into virus-like particles.

In some embodiments, the at least one RNA polynucleotide encodes an antigenic polypeptide that is responsible for binding of the KSHV to a cell being infected.

### G. Merkel Cell Polyomavirus (MCPyV)

In another embodiment, the oncoviral antigen is from Merkel Cell Polyomavirus (MCPyV). Merkel cell polyomavirus (MCPyV) is a non-enveloped, double-stranded DNA virus of the Polyomaviridae family and is an etiological agent of Merkel cell carcinoma (MCC). MCC is a rare, but aggressive, form of skin cancer, associated with advanced age, excessive UV exposure, immune deficiencies, and the presence of MCPyV. Approximately 1,500 new cases of MCC are diagnosed per year in the US, representing a relatively rare cancer; however, the incidence of MCC has tripled in the last two decades and annual diagnoses continue to climb by 5-10%. Despite its rarity, MCC is one of the most lethal and aggressive skin cancers with a mortality rate greater than 30% (Agelli and Clegg (2003) J Am Acad Dermatol 49:832-841; Becker et al., (2009) Cell Mol Life Sci 66:1-8; Calder and Smoller (2010) Adv Anat Pathol 17:155-161; Hodgson, (2005) J Sur Oncol 89:1-4; Lemos and Nghiem, (2007) J Invest Dermatol 127:2100-2103). Accordingly, in another aspect, an immune potentiator construct can be used to enhance an immune response against one or more Merkel Cell Polyomavirus (MCPyV) antigens of interest. For example, an antigen(s) of interest from MCPyV can be encoded by a chemically modified mRNA (mmRNA), provided on the same mmRNA as the immune potentiator construct or provided on a different construct mmRNA construct as the immune potentiator. The immune potentiator and MCPyV antigen mmRNAs can be formulated (or coformulated) and administered (simultaneously or sequentially) to a subject in need thereof to stimulate an immune response against the MCPyV antigen in the subject.

MCC is derived from malignant transformation of Merkel cells (alternatively Merkel-Ranvier cells or tactile epithelial cells), which are mechanoreceptive cells involved in touch and/or tactile sensation (Woo et al., (2016) Trends Cell Biol 25(2):74-81). MCPyV and is present in 80%-85% of clinical MCC tumor specimens (Feng et al., (2008) Science 319:1096-1100; Dalianis and Hirsch (2013) Virology 437:63-72, and references therein). MCPyV is considered the only human polyomavirus to date to cause tumors in its natural host (Arora et al., (2012) Curr. Opin. Virol 2:489-498; Spurgeon and Lambert (2013) Virology 435:118-130).

MCPyV viral DNA is clonally integrated in 80%-85% of MCC tumors. The prototype virus (MCV350) genome is a circular, double-stranded DNA molecule comprising 5387 base-pairs. The genomes of all MCPyV strains sequenced average ˜5.4 kilobases. The MCPyV genome contains early and late coding regions, expressed bidirectionally, and separated by a non-coding regulatory region that contains the viral origin of replication. The MCPyV early region (alternatively “T antigen locus”) is approximately 3 kb in size and encodes genes that are the first to be expressed upon infection (Feng et al., (2011) PLOS ONE 6:e22468; Feng et al., (2008) Science 319:1096-1100; Neumann et al., (2011) PLOS ONE 6:e29112). The MCPyV early region expresses three T antigens (proteins): large T antigen (LT), small T antigen (sT), and 57kT antigen (57kT) (Shuda et al., (2009) Int J Cancer 125(6):1243-9; Shuda et al., (2008) Proc Natl Acad Sci USA 105(42):16272-7). In addition to the three T antigens, the MCPyV early gene locus also encodes a fourth protein, the alternative T antigen open reading frame (ALTO). ALTO is transcribed from the 200 amino acid MUR region of LT, and seems to be evolutionarily related to the middle T antigen of the murine polyomavirus (Carter et al., (2013) Proc Natl Acad Sci USA 110:12744-12749).

The late region of the MCPyV encodes open reading frames for the major capsid protein viral protein 1 (VP1) and the minor capsid proteins 2 and 3 (VP2 and VP3). The MCPyV genome expresses a 22-nucleotide viral miRNA (MCV-miR-M1-5p) from the late strand that most likely autoregulates early viral gene expression during the late phase of infection (Lee et al., (2011) J Clin Virol 52 (3): 272-5; Seo et al., (2009) Virology 383 (2): 183-7). Studies support that constitutive expression of viral T antigens is required for virus-induced transformation (Spurgeon and Lambert (2013) Virology 435 (1): 118-130 and references therein).

In some embodiments, a RNA (e.g., mRNA) vaccine (e.g., comprising an immune potentiator construct and a MCPyV antigen construct, on the same or different mRNAs) comprises at least one RNA (e.g., mRNA) polynucleotide having an open reading frame encoding at least one MCPyV antigenic polypeptide or an immunogenic fragment thereof (e.g., an immunogenic fragment capable of inducing an immune response to MCPyV). In some embodiments, the at least one MCPyV antigenic polypeptide or immunogenic fragment thereof is selected from large T antigen (LT), small T antigen (sT), 57kT antigen (57kT), alternative T antigen (ALTO), major capsid protein viral protein 1 (VP1), the minor capsid viral proteins 2 or 3 (VP2 or VP3), and combinations thereof.

Some embodiments of the disclosure concern methods of treating and/or preventing MCPyV infection in humans, wherein one or more of the compositions described herein, which contain one or more immunomodulatory therapeutic nucleic acids encoding an immune potentiator construct and at least one MCPyV polypeptide or an immunogenic fragment thereof, that have been shown or are predicted by one skilled in the art to produce an immune response, is provided to a subject in need thereof (e.g. a person that is infected with or who is at risk of infection by MCPyV).

In some embodiments, the disclosure concerns methods of treating and/or preventing cancer resulting from and/or causally associated with MCPyV infection, wherein one or more of the compositions described herein, which contain one or more immunomodulatory therapeutic nucleic acids encoding an immune potentiator construct and at least one MCPyV polypeptide or an immunogenic fragment thereof, that have been shown or are predicted by one skilled in the art to produce an immune response, is provided to a subject in need thereof (e.g. a person that is infected with or who is at risk of infection by MCPyV).

Optionally, a subject in need of a medicament that prevents and/or treats MCPyV infection is provided a medicament comprising one or more of the immunomodulatory therapeutic nucleic acids encoding an immune potentiator construct and at least one MCPyV polypeptide or an immunogenic fragment thereof, to produce an immune response directed toward MCPyV and/or to the subject's cells that are infected with MCPyV. In some embodiments, the immune response results in a reduction in MCPyV viral titer. In some embodiments, the immune response results in the production of neutralizing anti-MCPyV antibodies. In some embodiments, the immune response results in a cytotoxic T-cell response directed at MCPyV infected cells.

In some embodiments, an immunomodulatory therapeutic nucleic acid (e.g., messenger RNA, mRNA) comprises at least one (e.g., mRNA) polynucleotide having an open reading frame encoding at least one MCPyV antigenic polypeptide or an immunogenic fragment thereof (e.g., an immunogenic fragment capable of inducing an immune response to MCPyV). In some embodiments, the at least one antigenic polypeptide or immunogenic fragment thereof is selected from large T antigen (LT), small T antigen (sT), 57kT antigen (57kT), alternative T antigen (ALTO), major capsid protein viral protein 1 (VP1), the minor capsid viral proteins 2 or 3 (VP2 or VP3), and combinations thereof.

In some embodiments, the at least one antigenic polypeptide or immunogenic fragment thereof is selected from provisional and/or confirmed MCPyV genotypes and/or subtypes (e.g. see Martel-Jantin et al., (2014) J Clin Microbiol 52 (5): 1687-1690; Hashida et al., 2014 J. Gen. Virol. 95:135-141; Matsushita et al., (2014) Virus Genes 48:233-242; Baez et al., (2016) Virus Res 221:1-7 herein incorporated in their entirety by reference). In some embodiments, the at least one antigenic polypeptide or immunogenic fragment thereof is selected from unassigned MCPyV isolates.

In some embodiments, the at least one RNA polynucleotide encodes an antigenic polypeptide that structurally modifies an infected cell.

In some embodiments, the at least one RNA polynucleotide encodes an antigenic polypeptide that forms part or all of the MCPyV viral capsid.

In some embodiments, the at least one RNA polynucleotide encodes an antigenic polypeptide that is capable of self-assembling into virus-like particles.

In some embodiments, the at least one RNA polynucleotide encodes an antigenic polypeptide that is responsible for binding of the MCPyV virus to a cell being infected.

### Personalized Cancer Vaccines

In some aspects, the present disclosure provides a personalized cancer vaccine comprising one or more mRNA constructs, wherein the one or more mRNA constructs encodes a polypeptide that enhances an immune response (i.e., immune potentiator) to a cancer antigen of interest. In some embodiments, the cancer antigen of interest is encoded by either the same or a separate mRNA construct. In some embodiments, the cancer antigen of interest is specific for a subject. For example, a cancer antigen of interest (e.g., selected and/or designed as described below) can be encoded by a chemically modified mRNA (mmRNA), provided on the same mmRNA as the immune potentiator construct or provided on a different mmRNA construct as the immune potentiator. The immune potentiator and cancer antigen mmRNAs can be formulated (or coformulated) and administered (simultaneously or sequentially) to a subject in need thereof to stimulate an immune response against the cancer antigen in the subject. Suitable cancer antigens, including personalized antigens specific for a cancer subject, for use with the immune potentiators are described herein.

For instance, the vaccine may include mRNA encoding for one or more cancer antigens specific for each subject, referred to as neoepitopes. Antigens that are expressed in or by tumor cells are referred to as “tumor associated antigens”. A particular tumor associated antigen may or may not also be expressed in non-cancerous cells. Many tumor mutations are well known in the art. Tumor associated antigens that are not expressed or rarely expressed in non-cancerous cells, or whose expression in non-cancerous cells is sufficiently reduced in comparison to that in cancerous cells and that induce an immune response induced upon vaccination, are referred to as neoepitopes. Neoepitopes are completely foreign to the body and thus would not produce an immune response against healthy tissue or be masked by the protective components of the immune system. In some embodiments personalized vaccines based on neoepitopes are desirable because such vaccine formulations will maximize specificity against a patient's specific tumor. Mutation-derived neoepitopes can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and translocations.

Methods for generating personalized cancer vaccines generally involve identification of mutations, e.g., using deep nucleic acid or protein sequencing techniques, identification of neoepitopes, e.g., using application of validated peptide-MHC binding prediction algorithms or other analytical techniques to generate a set of candidate T cell epitopes that may bind to patient HLA alleles and are based on mutations present in tumors, optional demonstration of antigen-specific T cells against selected neoepitopes or demonstration that a candidate neoepitope is bound to HLA proteins on the tumor surface and development of the vaccine.

Examples of techniques for identifying mutations include but are not limited to dynamic allele-specific hybridization (DASH), microplate array diagonal gel electrophoresis (MADGE), pyrosequencing, oligonucleotide-specific ligation, the TaqMan system as well as various DNA “chip” technologies i.e. Affymetrix SNP chips, and methods based on the generation of small signal molecules by invasive cleavage followed by mass spectrometry or immobilized padlock probes and rolling-circle amplification.

The deep nucleic acid or protein sequencing techniques are known in the art. Any type of sequence analysis method can be used. For instance nucleic acid sequencing may be performed on whole tumor genomes, tumor exomes (protein-encoding DNA) or tumor transcriptomes. Real-time single molecule sequencing-by-synthesis technologies rely on the detection of fluorescent nucleotides as they are incorporated into a nascent strand of DNA that is complementary to the template being sequenced. Other rapid high throughput sequencing methods also exist. Protein sequencing may be performed on tumor proteomes. Additionally, protein mass spectrometry may be used to identify or validate the presence of mutated peptides bound to MHC proteins on tumor cells. Peptides can be acid-eluted from tumor cells or from HLA molecules that are immunoprecipitated from tumor, and then identified using mass spectrometry. The results of the sequencing may be compared with known control sets or with sequencing analysis performed on normal tissue of the patient.

In some embodiments, these neoepitopes bind to class I HLA proteins with a greater affinity than the wild-type peptide and/or are capable of activating anti-tumor CD8 T-cells. Identical mutations in any particular gene are rarely found across tumors.

Proteins of MHC class I are present on the surface of almost all cells of the body, including most tumor cells. The proteins of MHC class I are loaded with antigens that usually originate from endogenous proteins or from pathogens present inside cells, and are then presented to cytotoxic T-lymphocytes (CTLs). T-Cell receptors are capable of recognizing and binding peptides complexed with the molecules of MHC class I. Each cytotoxic T-lymphocyte expresses a unique T-cell receptor which is capable of binding specific MHC/peptide complexes.

Using computer algorithms, it is possible to predict potential neoepitopes such as T-cell epitopes, i.e. peptide sequences, which are bound by the MHC molecules of class I or class II in the form of a peptide-presenting complex and then, in this form, recognized by the T-cell receptors of T-lymphocytes. Examples of programs useful for identifying peptides which will bind to MHC include for instance: Lonza Epibase, SYFPEITHI (Rammensee et al., Immunogenetics, 50 (1999), 213-219) and HLA_BIND (Parker et al., J. Immunol., 152 (1994), 163-175).

Once putative neoepitopes are selected, they can be further tested using in vitro and/or in vivo assays. Conventional in vitro lab assays, such as Elispot assays may be used with an isolate from each patient, to refine the list of neoepitopes selected based on the algorithm's predictions.

In some embodiments the mRNA cancer vaccines and vaccination methods include epitopes or antigens based on specific mutations (neoepitopes) and those expressed by cancer-germline genes (antigens common to tumors found in multiple patients, referred to herein as “traditional cancer antigens” or “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

In some embodiments, the vaccines may further include mRNA encoding for one or more non-mutated tumor antigens. In some embodiments, the vaccines may further include mRNA encoding for one or more mutated tumor antigens.

Many tumor antigens are known in the art. In some embodiments, the cancer or tumor antigen is one of the following antigens: CD2, CD19, CD20, CD22, CD27, CD33, CD37, CD38, CD40, CD44, CD47, CD52, CD56, CD70, CD79, CD137, 4—IBB, 5T4, AGS-5, AGS-16, Angiopoietin 2, B7.1, B7.2, B7DC, B7H1, B7H2, B7H3, BT-062, BTLA, CAIX, Carcinoembryonic antigen, CTLA4, Cripto, ED-B, ErbB1, ErbB2, ErbB3, ErbB4, EGFL7, EpCAM, EphA2, EphA3, EphB2, FAP, Fibronectin, Folate Receptor, Ganglioside GM3, GD2, glucocorticoid-induced tumor necrosis factor receptor (GITR), gp100, gpA33, GPNMB, ICOS, IGFIR, Integrin av, Integrin αvβ, LAG-3, Lewis Y, Mesothelin, c-MET, MN Carbonic anhydrase IX, MUC1, MUC16, Nectin-4, NKGD2, NOTCH, OX40, OX40L, PD-1, PDL1, PSCA, PSMA, RANKL, ROR1, ROR2, SLC44A4, Syndecan-1, TACI, TAG-72, Tenascin, TIM3, TRAILR1, TRAILR2, VEGFR-1, VEGFR-2, VEGFR-3, and variants thereof.

An epitope, also known as an antigenic determinant, as used herein is a portion of an antigen that is recognized by the immune system in the appropriate context, specifically by antibodies, B cells, or T cells. Epitopes include B cell epitopes and T cell epitopes. B-cell epitopes are peptide sequences which are required for recognition by specific antibody producing B-cells. B cell epitopes refer to a specific region of the antigen that is recognized by an antibody. The portion of an antibody that binds to the epitope is called a paratope. An epitope may be a conformational epitope or a linear epitope, based on the structure and interaction with the paratope. A linear, or continuous, epitope is defined by the primary amino acid sequence of a particular region of a protein. The sequences that interact with the antibody are situated next to each other sequentially on the protein, and the epitope can usually be mimicked by a single peptide. Conformational epitopes are epitopes that are defined by the conformational structure of the native protein. These epitopes may be continuous or discontinuous, i.e. components of the epitope can be situated on disparate parts of the protein, which are brought close to each other in the folded native protein structure.

T-cell epitopes are peptide sequences which, in association with proteins on APC, are required for recognition by specific T-cells. T cell epitopes are processed intracellularly and presented on the surface of APCs, where they are bound to MHC molecules including MHC class II and MHC class I.

In other aspects, the cancer vaccine of the invention comprises an mRNA vaccine encoding multiple peptide epitope antigens, arranged with one or more interspersed universal type II T-cell epitopes. The universal type II T-cell epitopes, include, but are not limited to ILMQYIKANSKFIGI (Tetanus toxin; SEQ ID NO: 226), FNNFTVSFWLRVPKVSASHLE, (Tetanus toxin; SEQ ID NO: 227), QYIKANSKFIGITE (Tetanus toxin; SEQ ID NO: 228) QSIALSSLMVAQAIP (Diptheria toxin; SEQ ID NO: 229), and AKFVAAWTLKAAA (pan-DR epitope (PADRE); SEQ ID NO: 230). In some embodiments, the mRNA vaccine comprises the same universal type II T-cell epitope. In other embodiments, the mRNA vaccine comprises 2, 3, 4, 5, 6, 7, 8, 9, 10, 15, or 20 different universal type II T-cell epitopes. In some embodiments, the one or more universal type II T-cell epitope(s) are interspersed between every cancer antigen. In other embodiments, the one or more universal type II T-cell epitope(s) are interspersed between every 2, 3, 4, 5, 6, 7, 8, 9, 10, 11, 12, 13, 14, 15, 16, 17, 18, 19, 20, 22, 24, 26, 28, 30, 35, 40, 45, 50, 55, 60, 65, 70, 75, 80, 85, 90, or 100 cancer antigens.

Epitopes can be identified using a free or commercial database (Lonza Epibase, antitope for example). Such tools are useful for predicting the most immunogenic epitopes within a target antigen protein. The selected peptides may then be synthesized and screened in human HLA panels, and the most immunogenic sequences are used to construct the mRNAs encoding the antigen(s). One strategy for mapping epitopes of Cytotoxic T-Cells based on generating equimolar mixtures of the four C-terminal peptides for each nominal 11-mer across a protein. This strategy would produce a library antigen containing all the possible active CTL epitopes.

The peptide epitope may be any length that is reasonable for an epitope. In some embodiments the peptide epitope is 9-30 amino acids. In other embodiments the length is 9-22, 9-29, 9-28, 9-27, 9-26, 9-25, 9-24, 9-23, 9-21, 9-20, 9-19, 9-18, 10-22, 10-21, 10-20, 11-22, 22-21, 11-20, 12-22, 12-21, 12-20,13-22, 13-21, 13-20, 14-19, 15-18, or 16-17 amino acids.

The personalized cancer vaccines include multiple epitopes. In some embodiments, the personalized cancer vaccines encode 48-54 personalized cancer antigens. In one embodiment, the personalized cancer vaccines encode 52 personalized cancer antigens. In some embodiments, each of the personalized cancer antigens is encoded by a separate open reading frame. In some embodiments the personalized cancer vaccines are composed of 45 or more, 46 or more, 47 or more, 48 or more, 49 or more, 50 or more, 51 or more, 52 or more, 53 or more, 54 or more, or 55 or more antigens. In other embodiments the personalized cancer vaccines are composed of 1000 or less, 900 or less, 500 or less, 100 or less, 75 or less, 50 or less, 40 or less, 30 or less, 20 or less or 100 or less epitopes. In yet other embodiments the personalized cancer vaccines have 3-100, 5-100, 10-100, 15-100, 20-100, 25-100, 30-100, 35-100, 40-100, 45-100, 50-100, 55-100, 60-100, 65-100, 70-100, 75-100, 80-100, 90-100, 5-50, 10-50, 15-50, 20-50, 25-50, 30-50, 35-50, 40-50, 45-50, 100-150, 100-200, 100-300, 100-400, 100-500, 50-500, 50-800, 50-1,000, or 100-1,000 cancer antigens.

In some embodiments, the optimal length of a peptide epitope may be obtained through the following procedure: synthesizing a V5 tag concatemer-test protease site, introducing it into DC cells (for example, using an RNA Squeeze procedure), lysing the cells, and then running an anti-V5 Western blot to assess the cleavage at protease sites.

The RNA Squeeze technique is an intracellular delivery method by which a variety of materials can be delivered to a broad range of live cells. Cells are subjected to microfluidic construction, which causes rapid mechanical deformation. The deformation results in temporary membrane disruption and the newly-formed transient pores. Material is then passively diffused into the cell cytosol via the transient pores. The technique can be used in a variety of cell types, including primary fibroblasts, embryonic stem cells, and a host of immune cells, and has been shown to have relatively high viability in most applications and does not damage sensitive materials, such as quantum dots or proteins, through its actions. Sharei et al., PNAS (2013); 110 (6): 2082-7.

The neoepitopes may be designed to optimally bind to MHC in order to promote a robust immune response. In some embodiments each peptide epitope comprises an antigenic region and a MHC stabilizing region. An MHC stabilizing region is a sequence which stabilizes the peptide in the MHC. The MHC stabilizing region may be 5-10, 5-15, 8-10, 1-5, 3-7, or 3-8 amino acids in length. In yet other embodiments the antigenic region is 5-100 amino acids in length. The peptides interact with the molecules of MHC class I by competitive affinity binding within the endoplasmic reticulum, before they are presented on the cell surface. The affinity of an individual peptide is directly linked to its amino acid sequence and the presence of specific binding motifs in defined positions within the amino acid sequence. The peptide being presented in the MHC is held by the floor of the peptide-binding groove, in the central region of the α1/α2 heterodimer (a molecule composed of two nonidentical subunits). The sequence of residues, of the peptide-binding groove's floor determines which particular peptide residues it binds.

Optimal binding regions may be identified by a computer assisted comparison of the affinity of a binding site (MHC pocket) for a particular amino acid at each amino acid in the binding site for each of the target epitopes to identify an ideal binder for all of the examined antigens. The MHC stabilization regions of the epitopes may be identified using amino acid prediction matrices of data points for a binding site. An amino acid prediction matrix is a table having a first and a second axis defining data points. Prediction matrices can be generated as shown in Singh, H. and Raghava, G.P.S. (2001), “ProPred: prediction of HLA-DR binding sites.” Bioinformatics, 17 (12), 1236-37).

In some embodiments the MHC stabilizing region is designed based on the subject's particular MHC. In that way the MHC stabilizing region can be optimized for each patient.

In some instances each epitope of an antigen may include a MHC stabilizing region. All of the MHC stabilizing regions within the epitopes may be the same or they may be different. The MHC stabilizing regions may be at the N terminal portion of the peptide or the C terminal portion of the peptide. Alternatively the MHC stabilizing regions may be in the central region of the peptide. The neoepitopes in some embodiments are 13 residues or less in length and usually consist of between about 8 and about 11 residues, particularly 9 or 10 residues. In other embodiments the neoepitopes may be designed to be longer. For instance, the neoepitopes may have extensions of 2-5 amino acids toward the N- and C-terminus of each corresponding gene product. The use of a longer peptide may allow endogenous processing by patient cells and may lead to more effective antigen presentation and induction of T cell responses.

The neoepitopes selected for inclusion in the vaccine typically will be high affinity binding peptides. In some aspect the neoepitope binds an HLA protein with greater affinity than a wild-type peptide. The neoepitope has an IC50 of at least less than 5000 nM, at least less than 500 nM, at least less than 250 nM, at least less than 200 nM, at least less than 150 nM, at least less than 100 nM, at least less than 50 nM or less in some embodiments. Typically, peptides with predicted IC50<50 nM, are generally considered medium to high affinity binding peptides and will be selected for testing their affinity empirically using biochemical assays of HLA-binding. Finally, it will be determined whether the human immune system can mount effective immune responses against these mutated tumor antigens and thus effectively kill tumor but not normal cells.

Neoepitopes having the desired activity may be modified as necessary to provide certain desired attributes, e.g. improved pharmacological characteristics, while increasing or at least retaining substantially all of the biological activity of the unmodified peptide to bind the desired MHC molecule and activate the appropriate T cell or B cell. For instance, the neoepitopes may be subject to various changes, such as substitutions, either conservative or non-conservative, where such changes might provide for certain advantages in their use, such as improved MHC binding. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, Ill., Pierce), 2d Ed. (1984).

The neoepitopes can also be modified by extending or decreasing the compound's amino acid sequence, e.g., by the addition or deletion of amino acids. The peptides, polypeptides or analogs can also be modified by altering the order or composition of certain residues, it being readily appreciated that certain amino acid residues essential for biological activity, e.g., those at critical contact sites or conserved residues, may generally not be altered without an adverse effect on biological activity.

Typically, a series of peptides with single amino acid substitutions are employed to determine the effect of electrostatic charge, hydrophobicity, etc. on binding. For instance, a series of positively charged (e.g., Lys or Arg) or negatively charged (e.g., Glu) amino acid substitutions are made along the length of the peptide revealing different patterns of sensitivity towards various MHC molecules and T cell or B cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an MHC molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding.

The neoepitopes may also comprise isosteres of two or more residues in the neoepitopes. An isostere as defined here is a sequence of two or more residues that can be substituted for a second sequence because the steric conformation of the first sequence fits a binding site specific for the second sequence. The term specifically includes peptide backbone modifications well known to those skilled in the art. Such modifications include modifications of the amide nitrogen, the .alpha.-carbon, amide carbonyl, complete replacement of the amide bond, extensions, deletions or backbone crosslinks. See, generally, Spatola, Chemistry and Biochemistry of Amino Acids, Peptides and Proteins, Vol. VII (Weinstein ed., 1983).

The consideration of the immunogenicity is an important component in the selection of optimal neoepitopes for inclusion in a vaccine. Immunogenicity may be assessed for instance, by analyzing the MHC binding capacity of a neoepitope, HLA promiscuity, mutation position, predicted T cell reactivity, actual T cell reactivity, structure leading to particular conformations and resultant solvent exposure, and representation of specific amino acids. Known algorithms such as the NetMHC prediction algorithm can be used to predict capacity of a peptide to bind to common HLA-A and —B alleles. Structural assessment of a MHC bound peptide may also be conducted by in silico 3-dimensional analysis and/or protein docking programs. Use of a predicted epitope structure when bound to a MHC molecule, such as acquired from a Rosetta algorithm, may be used to evaluate the degree of solvent exposure of an amino acid residues of an epitope when the epitope is bound to a MHC molecule. T cell reactivity may be assessed experimentally with epitopes and T cells in vitro. Alternatively T cell reactivity may be assessed using T cell response/sequence datasets.

An important component of a neoepitope included in a vaccine, is a lack of self-reactivity. The putative neoepitopes may be screened to confirm that the epitope is restricted to tumor tissue, for instance, arising as a result of genetic change within malignant cells. Ideally, the epitope should not be present in normal tissue of the patient and thus, self-similar epitopes are filtered out of the dataset.

In other aspects the disclosure provides a method for preparing a mRNA cancer vaccine, by isolating a sample from a subject, identifying a plurality of cancer antigens in the sample, determining T-cell epitopes from the plurality of cancer antigens, preparing a mRNA cancer vaccine having an open reading frame encoding an antigen and a polypeptide that enhances an immune response to the antigen, wherein the antigen comprises at least one of the T-cell epitopes. In some embodiments the method further involves determining binding strength of the T-cell epitopes to a MHC of a subject. In other embodiments the method further involves determining a T-cell receptor face (TCR face) for each epitope and selecting epitopes having a TCR face with low similarity to endogenous proteins. The T-cell epitopes may have been optimized for binding strength to a MHC of the subject is provided. In some embodiments a TCR face for each epitope has a low similarity to endogenous proteins.

For instance a technology referred to as JanusMatrix (Epivax), which examines cross-reactive T cell epitopes from both HLA binding and TCR-facing sides to allow comparison across large genome sequence databases can be used to identify epitopes having a desirable TCR face and binding strength to MHC. A suite of algorithms can be used alone or together with the JanusMatrix to optimize epitope selection. For example EpiMatrix takes overlapping 9-mer frames derived from the conserved target protein sequences and scores them for potential binding affinity against a panel of Class I or Class II HLA alleles; each frame-by-allele assessment that scores highly and is predicted to bind is a putative T cell epitope. ClustiMer takes EpiMatrix output and identifies clusters of 9-mers that contain large numbers of putative T cell epitopes. BlastiMer automates the process of submitting the previously identified sequences to BLAST to determine if any share similarities with the human genome; any such similar sequences would be likely to be tolerated or to elicit an unwanted autoimmune response. EpiAssembler takes the conserved, immunogenic sequences identified by Conservatrix and EpiMatrix and knits them together to form highly immunogenic consensus sequences. JanusMatrix can be used to screen out sequences which could potentially elicit an undesired autoimmune or regulatory T cell response due to homology with sequences encoded by the human genome. VaccineCAD can be used to link candidate epitopes into a string-of-beads design while minimizing nonspecific junctional epitopes that may be created in the linking process.

Methods for generating personalized cancer vaccines according to the disclosure involve identification of mutations using techniques such as deep nucleic acid or protein sequencing methods as described herein of tissue samples. In some embodiments an initial identification of mutations in a patient's transcriptome is performed. The data from the patient's transcriptome is compared with sequence information from the patients exome in order to identify patient specific and tumor specific mutations that are expressed. The comparison produces a dataset of putative neoepitopes, referred to as a mutanome. The mutanome may include approximately 100-10,000 candidate mutations per patients. The mutanome is subject to a data probing analysis using a set of inquiries or algorithms to identify an optimal mutation set for generation of a neoantigen vaccine. In some embodiments an mRNA neoantigen vaccine is designed and manufactured. The patient is then treated with the vaccine.

In some embodiments the entire method from the initiation of the mutation identification process to the start of patient treatment is achieved in less than 2 months. In other embodiments the whole process is achieved in 7 weeks or less, 6 weeks or less, 5 weeks or less, 4 weeks or less, 3 weeks or less, 2 weeks or less or less than 1 week. In some embodiments the whole method is performed in less than 30 days.

The mutation identification process may involve both transcriptome and exome analysis or only transcriptome or exome analysis. In some embodiments transcriptome analysis is performed first and exome analysis is performed second. The analysis is performed on a biological or tissue sample. In some embodiments a biological or tissue sample is a blood or serum sample. In other embodiments the sample is a tissue bank sample or EBV transformation of B-cells.

Once an mRNA vaccine is synthesized, it is administered to the patient. In some embodiments the vaccine is administered on a schedule for up to two months, up to three months, up to four month, up to five months, up to six months, up to seven months, up to eight months, up to nine months, up to ten months, up to eleven months, up to 1 year, up to 1 and ½ years, up to two years, up to three years, or up to four years. The schedule may be the same or varied. In some embodiments the schedule is weekly for the first 3 weeks and then monthly thereafter.

At any point in the treatment the patient may be examined to determine whether the mutations in the vaccine are still appropriate. Based on that analysis the vaccine may be adjusted or reconfigured to include one or more different mutations or to remove one or more mutations.

It has been recognized and appreciated that, by analyzing certain properties of cancer associated mutations, optimal neoepitopes may be assessed and/or selected for inclusion in an mRNA vaccine. A property of a neoepitope or set of neoepitopes may include, for instance, an assessment of gene or transcript-level expression in patient RNA-seq or other nucleic acid analysis, tissue-specific expression in available databases, known oncogenes/tumor suppressors, variant call confidence score, RNA-seq allele-specific expression, conservative vs. non-conservative AA substitution, position of point mutation (Centering Score for increased TCR engagement), position of point mutation (Anchoring Score for differential HLA binding), Selfness: <100% core epitope homology with patient WES data, HLA-A and —B IC50 for 8mers-11mers, HLA-DRB1 IC50 for 15mers-20mers, promiscuity Score (i.e. number of patient HLAs predicted to bind), HLA-C IC50 for 8mers-11mers, HLA-DRB3-5 IC50 for 15mers-20mers, HLA-DQB1/A1 IC50 for 15mers-20mers, HLA-DPB1/A1 IC50 for 15mers-20mers, Class I vs Class II proportion, Diversity of patient HLA-A, —B and DRB1 allotypes covered, proportion of point mutation vs complex epitopes (e.g. frameshifts), and/or pseudo-epitope HLA binding scores.

In some embodiments, the properties of cancer associated mutations used to identify optimal neoepitopes are properties related to the type of mutation, abundance of mutation in patient sample, immunogenicity, lack of self-reactivity, and nature of peptide composition.

The type of mutation should be determined and considered as a factor in determining whether a putative epitope should be included in a vaccine. The type of mutation may vary. In some instances it may be desirable to include multiple different types of mutations in a single vaccine. In other instances a single type of mutation may be more desirable. A value for particular mutation can be weighted and calculated.

The abundance of the mutation in a patient sample may also be scored and factored into the decision of whether a putative epitope should be included in a vaccine. Highly abundant mutations may promote a more robust immune response.

In some embodiments, the personalized mRNA cancer vaccines described herein may be used for treatment of cancer.

mRNA cancer vaccines may be administered prophylactically or therapeutically as part of an active immunization scheme to healthy individuals or early in cancer or late stage and/or metastatic cancer. In one embodiment, the effective amount of the mRNA cancer vaccine provided to a cell, a tissue or a subject may be enough for immune activation, and in particular antigen specific immune activation.

In some embodiments, the mRNA cancer vaccine may be administered with an anti-cancer therapeutic agent, including but not limited to, a traditional cancer vaccine. The mRNA cancer vaccine and anti-cancer therapeutic can be combined to enhance immune therapeutic responses even further. The mRNA cancer vaccine and other therapeutic agent may be administered simultaneously or sequentially. When the other therapeutic agents are administered simultaneously they can be administered in the same or separate formulations, but are administered at the same time. The other therapeutic agents are administered sequentially with one another and with the mRNA cancer vaccine, when the administration of the other therapeutic agents and the mRNA cancer vaccine is temporally separated. The separation in time between the administration of these compounds may be a matter of minutes or it may be longer, e.g. hours, days, weeks, months. Other therapeutic agents include but are not limited to anti-cancer therapeutic, adjuvants, cytokines, antibodies, antigens, etc.

In another embodiment, the peptide epitopes are in the form of a concatemeric cancer antigen comprised of 2-100 peptide epitopes. In some embodiments, the concatemeric cancer antigen comprises one or more of: a) the 2-100 peptide epitopes are interspersed by cleavage sensitive sites; b) the mRNA encoding each peptide epitope is linked directly to one another without a linker; c) the mRNA encoding each peptide epitope is linked to one or another with a single nucleotide linker; d) each peptide epitope comprises 25-35 amino acids and includes a centrally located SNP mutation; e) at least 30% of the peptide epitopes have a highest affinity for class I MHC molecules from a subject; f) at least 30% of the peptide epitopes have a highest affinity for class II MHC molecules from a subject; g) at least 50% of the peptide epitopes have a predicated binding affinity of IC >500 nM for HLA-A, HLA-B and/or DRB1; h) the mRNA encodes 45-55 peptide epitopes; i) the mRNA encodes 52 peptide epitopes; j) 50% of the peptide epitopes have a binding affinity for class I MHC and 50% of the peptide epitopes have a binding affinity for class II MHC; k) the mRNA encoding the peptide epitopes is arranged such that the peptide epitopes are ordered to minimize pseudo-epitopes, 1) at least 30% of the peptide epitopes are class I MHC binding peptides of 15 amino acids in length; and/or m) at least 30% of the peptide epitopes are class II MHC binding peptides of 21 amino acids in length.

### Bacterial Vaccines

In some aspects, the present disclosure provides a bacterial vaccine comprising one or more mRNA constructs, wherein the one or more mRNA constructs encodes a polypeptide that enhances an immune response (i.e., immune potentiator) to a bacterial antigen of interest. In some embodiments, the bacterial antigen of interest is encoded by either the same or separate mRNA construct. In some embodiments, the bacterial vaccine comprises one or more mRNA constructs encoding a polypeptide that enhances an immune response, and one or more mRNA constructs encoding at least one bacterial antigen of interest. For example, a bacterial antigen of interest can be encoded by a chemically modified mRNA (mmRNA), provided on the same mmRNA as the immune potentiator construct or provided on a different mmRNA construct as the immune potentiator. The immune potentiator and bacterial antigen mmRNAs can be formulated (or coformulated) and administered (simultaneously or sequentially) to a subject in need thereof to stimulate an immune response against the bacterial antigen in the subject. Suitable bacterial antigens for use with the immune potentiators are described herein.

In some embodiments, the bacterial vaccine is prophylactic (i.e., prevents infection). In some embodiments, the bacterial vaccine is therapeutic (i.e., treats infection). In some embodiments, the bacterial vaccine induces a humoral immune response (i.e., production of antibodies specific for the bacterial antigen of interest). In some embodiments, the bacterial vaccine induces an adaptive immune response. An adaptive immune response occurs in response to confrontation with an antigen or immunogen, where the immune response is specific for antigenic determinants of the antigen/immunogen. Examples of adaptive immune responses are induction of antigen specific antibody production or antigen specific induction/activation of T helper lymphocytes or cytotoxic lymphocytes.

In some embodiments, the bacterial vaccine induces a protective, adaptive immune response, wherein an antigen-specific immune response is induced in a subject as a reaction to immunization (artificial or natural) with an antigen, where the immune response is capable of protecting the subject against subsequent challenges with the antigen or a pathology-related agent that includes the antigen.

In some embodiments, the bacterial vaccine described herein is used to treat an infection by Staphylococcus aureus. In some embodiments, the bacterial vaccine described herein is used to treat an infection by antibiotic resistant Staphylococcus aureus. In some embodiments, the bacterial vaccine described herein is used to treat an infection by Methicillin Resistant Staphylococcus aureus (MRSA).

Nosocomial infections are one of the most common and costly problems for the U.S. healthcare system, with S. aureus being the second-leading cause of such infections. MRSA is responsible for 40-50% of all nosocomially-acquired S. aureus infection. Further, recent studies indicate that S. aureus is also the major mediator of prosthetic implant infection. One of the most important mechanisms utilized by S. aureus to thwart the host immune response and develop into a persistent infection is through the formation of a highly-developed biofilm. A biofilm is a microbe-derived community in which bacterial cells are attached to a hydrated surface and embedded in a polysaccharide matrix. Bacteria in a biofilm exhibit an altered phenotype in their growth, gene expression, and protein production.

Accordingly, in some embodiments, the bacterial vaccines described herein prevent the establishment of biofilm-mediated chronic infections by S. aureus. In some embodiments, the antigen of interest if found in biofilm produced by S. aureus. Examples of such antigens are described in U.S. Pat. No. 9,265,820, herein incorporated by reference in its entirety. In some embodiments, the bacterial vaccine comprises at least one polypeptide expressed by a planktonic form of the bacteria, and at least one polypeptide expressed by the biofilm form of the bacteria.

In some embodiments, the bacterial antigen of interest is derived from S. aureus. Drug resistant S. aureus expresses a number of surface exposed proteins which are candidates as vaccine targets, as well as candidates as immunizing agents for preparation of antibodies that target S. aureus. Examples of such antigens are described in PCT Publication Nos. WO 2012/136653 and WO 2015/082536, and in Ramussen, K. et al, Vaccine, Vol. 34:4602-4609 (2016), each of which are herein incorporated by reference in its entirety.

The skilled artisan will understand that the identity, number and size of the different S. aureus proteins that can be encoded by an mRNA for the bacterial vaccines described herein, may vary. For example, the vaccine may comprise mRNA encoding only portions of the full-length polypeptides. In some embodiments, the vaccine may comprise mRNA encoding a combination of portions and full-length polypeptides.

The identity of the planktonic- and biofilm-expressed polypeptides encoded by the mRNA included in the bacterial vaccines described herein is not particularly limited, but each is a polypeptide from a strain of S. aureus. In some embodiments, the polypeptide is exposed on the surface of the bacteria.

In one embodiment, the bacterial antigen is a multivalent antigen (i.e., the antigen comprises multiple antigenic epitopes, such as multiple antigenic peptides comprising different epitopes, such as a concatermeric antigen).

In another embodiment, the bacterial antigen is a Chlamydia antigen, such as a MOMP, OmpA, OmpL, OmpF or OprF antigen. Suitable Chlamydia antigens are described further in PCT Application No. PCT/US2016/058314, the entire contents of which is expressly incorporated herein by reference.

### Multivalent Vaccines

An immune potentiator construct can be used in combination with a multivalent antigen (i.e., the antigen comprises multiple antigenic epitopes, such as multiple antigenic peptides comprising different epitopes, such as a concatermeric antigen) to thereby enhance an immune response against the multivalent antigen. In one embodiment, the multivalent antigen is a cancer antigen. In another embodiment, the multivalent antigen is a bacterial antigen. For example, a multivalent antigen of interest (e.g., designed as described below) can be encoded by a chemically modified mRNA (mmRNA), provided on the same mmRNA as the immune potentiator construct or provided on a different mmRNA construct as the immune potentiator. The immune potentiator and multivalent antigen mmRNAs can be formulated (or coformulated) and administered (simultaneously or sequentially) to a subject in need thereof to stimulate an immune response against the multivalent antigen in the subject. Suitable multivalent antigens, including cancer antigens and bacterial antigens, for use with the immune potentiators are described herein.

In some embodiments, the mRNA vaccines described herein comprise an mRNA having an open reading frame encoding a concatemeric antigen comprised of 2-100 peptide epitopes.

In some embodiments, the concatemeric vaccines described herein may include multiple copies of a single neoepitope, multiple different neoepitopes based on a single type of mutation, i.e. point mutation, multiple different neoepitopes based on a variety of mutation types, neoepitopes and other antigens, such as tumor associated antigens or recall antigens.

In some embodiments the concatemeric antigen may include a recall antigen, also sometimes referred to as a memory antigen. A recall antigen is an antigen that has previously been encountered by an individual and for which there are pre-existent memory lymphocytes. In some embodiments the recall antigen may be an infectious disease antigen that the individual has likely encountered such as an influenza antigen. The recall antigen helps promote a more robust immune response.

In addition to peptide epitopes, the concatemeric antigen may have one or more targeting sequences. A targeting sequence, as used herein, refers to a peptide sequence that facilitates uptake of the peptide into intracellular compartments such as endosomes for processing and/or presentation within MHC class I or II determinants.

The targeting sequence may be present at the N-terminus and/or C-terminus of an epitope of the concatemeric antigen, either directly adjacent thereto or separated by a linker of a cleavage sensitive site. Targeting sequences have a variety of lengths, for instance 4-50 amino acids in length.

The targeting sequence may be, for instance, an endosomal targeting sequence. An endosomal targeting sequence is a sequence derived from an endosomal or lysosomal protein known to reside in MHC class II Ag processing compartments, such as invariant chain, lysosome-associated membrane proteins (LAMP1,4 LAMP2), and dendritic cell (DC)-LAMP or a sequence having at least 80% sequence identity thereto. Additionally, an exemplary nucleic acid encoding a MHC class I signal peptide fragment (78 bp, secretion signal (sec)) and the transmembrane and cytosolic domains including the stop-codon (MHC class I trafficking signal (MITD), 168 bp) both amplified from activated PBMC, may be used (sec sense, 5′-aag ctt agc ggc cgc acc atg egg gtc acg geg ccc cga acc-3′ (SEQ ID NO: 1314); sec antisense, 5′-ctg cag gga gcc ggc cca ggt ctc ggt cag-3′ (SEQ ID NO: 1315); MITD sense, 5′-gga tcc atc gtg ggc att gtt gct ggc ctg gct-3′ (SEQ ID NO: 1316); and MITD antisense, 5′-gaa ttc agt ctc gag tca agc tgt gag aga cac atc aga gcc-3′ (SEQ ID NO: 1317).

MHC Class I presentation is typically an inefficient process (only 1 peptide of 10,000 degraded molecules is actually presented). Priming of CD8 T cells with APCs provides insufficient densities of surface peptide/MHC I complexes results in weak responders exhibiting impaired cytokine secretion and a decreased memory pool. The methods described herein are capable of increasing the efficiency of MHC Class I presentation. MHC class I targeting sequences include MHC Class I trafficking signal (MITD) and PEST sequences (increase antigen-specific CD8 T cell responses presumably by targeting proteins for rapid degradation).

In some embodiments the mRNA vaccines can be combined with agents for promoting the production of antigen presenting cells (APCs), for instance, by converting non-APCs into Pseudo-APCs. Antigen presentation is a key step in the initiation, amplification and duration of an immune response. In this process fragments of antigens are presented through the Major Histocompatibility Complex (MHC) or Human Leukocyte Antigens (HLA) to T cells driving an antigen-specific immune response. For immune prophylaxis and therapy, enhancing this response is important for improved efficacy. The mRNA vaccines of the invention may be designed or enhanced to drive efficient antigen presentation. One method for enhancing APC processing and presentation, is to provide better targeting of the mRNA vaccines to antigen presenting cells (APC). Another approach involves activating the APC cells with immune-stimulatory formulations and/or components.

Alternatively, methods for reprograming non-APC into becoming APC may be used with the mRNA vaccines described herein. Importantly, most cells that take up mRNA formulations and are targets of their therapeutic actions are not APC. Therefore, designing a way to convert these cells into APC would be beneficial for efficacy. Methods and approaches for delivering RNA vaccines, e.g., mRNA vaccines to cells while also promoting the shift of a non-APC to an APC are provided herein. In some embodiments a mRNA encoding an APC reprograming molecule is included in the mRNA vaccine or coadministered with the mRNA vaccine.

An APC reprograming molecule, as used herein, is a molecule that promotes a transition in a non APC cell to an APC-like phenotype. An APC-like phenotype is property that enables MHC class II processing. Thus, an APC cell having an APC-like phenotype is a cell having one or more exogenous molecules (APC reprograming molecule) which has enhanced MHC class II processing capabilities in comparison to the same cell not having the one or more exogenous molecules. In some embodiments an APC reprograming molecule is a CIITA (a central regulator of MHC Class II expression); a chaperone protein such as CLIP, HLA-DO, HLA-DM etc. (enhancers of loading of antigen fragments into MHC Class II) and/or a costimulatory molecule like CD40, CD80, CD86 etc. (enhancers of T cell antigen recognition and T cell activation).

A CIITA protein is a transactivator that enhances activation of transcription of MHC Class II genes (Steimle et al., 1993, Cell 75:135-146) by interacting with a conserved set of DNA binding proteins that associate with the class II promoter region. The transcriptional activation function of CIITA has been mapped to an amino terminal acidic domain (amino acids 26-137). A nucleic acid molecule encoding a protein that interacts with CIITA, termed CIITA-interacting protein 104 (also referred to herein as CIP104). Both CITTA and CIP104 have been shown to enhance transcription from MHC class II promoters and thus are useful as APC reprograming molecule of the invention. In some embodiments the APC reprograming molecule are full length CIITA, CIP104 or other related molecules or active fragments thereof, such as amino acids 26-137 of CIITA, or amino acids having at least 80% sequence identity thereto and maintaining the ability to enhance activation of transcription of MHC Class II genes.

In some embodiments the APC reprograming molecule is delivered to a subject in the form of an mRNA encoding the APC reprograming molecule. As such the mRNA vaccines described herein may include an mRNA encoding an APC reprograming molecule. In some embodiments the mRNA in monocistronic. In other embodiments it is polycistronic. In some embodiments the mRNA encoding the one or more antigens is in a separate formulation from the mRNA encoding the APC reprograming molecule. In other embodiments the mRNA encoding the one or more antigens is in the same formulation as the mRNA encoding the APC reprograming molecule. In some embodiments the mRNA encoding the one or more antigens is administered to a subject at the same time as the mRNA encoding the APC reprograming molecule. In other embodiments the mRNA encoding the one or more antigens is administered to a subject at a different time than the mRNA encoding the APC reprograming molecule. For instance, the mRNA encoding the APC reprograming molecule may be administered prior to the mRNA encoding the one or more antigens. The mRNA encoding the APC reprograming molecule may be administered immediately prior to, at least 1 hour prior to, at least 1 day prior to, at least one week prior to, or at least one month prior to the mRNA encoding the antigens. Alternatively, the mRNA encoding the APC reprograming molecule may be administered after the mRNA encoding the one or more antigens. The mRNA encoding the APC reprograming molecule may be administered immediately after, at least 1 hour after, at least 1 day after, at least one week after, or at least one month after the mRNA encoding the antigens.

In other embodiments, the targeting sequence is a ubiquitination signal that is attached at either or both ends of the encoded peptide. In other embodiments, the targeting sequence is a ubiquitination signal that is attached at an internal site of the encoded peptide and/or to either end. Thus, the mRNA may include a nucleic acid sequence encoding a ubiquitination signal at either or both ends of the nucleotides encoding the concatemeric peptide. Ubiquitination, a post-translational modification, is the process of attaching ubiquitin to a substrate target protein. A ubiquitination signal is a peptide sequence which enables the targeting and processing of a peptide to one or more proteasomes. By targeting and processing the peptide through the use of a ubiquitination signal the intracellular processing of the peptide can more closely recapitulate antigen processing in Antigen Presenting Cells (APCs).

Ubiquitin is an 8.5 kDa regulatory protein that is found in nearly all tissues of eukaryotic organisms. In the human genome, there are four genes that produce ubiquitin: UBB, UBC, UBA52, and RPS27A. UBA52 and RPS27A code for a single copy of ubiquitin fused to the ribosomal proteins L40 and S27a, respectively. The UBB and UBC genes code for polyubiquitin precursor proteins. There are three steps to ubiquitination, performed by three enzymes. Ubiquitin-activating enzymes, also called E1 enzymes, modify the ubiquitin so that it is in a reactive state. The E1 binds to both ATP and ubiquitin, catalyzing the acyl-adenylation of ubiquitin's C-terminal. Then, the ubiquitin is transferred to an active site cysteine residue, releasing AMP. Ultimately, a thioester linkage is formed between the ubiquitin's C-terminal carboxyl group and the E1 cysteine sulfhydryl group. In the human genome, UBA1 and UBA6 are the two genes that code for the E1 enzymes.

The activated ubiquitin is then subjected to E2 ubiquitin-conjugating enzymes, which transfer the ubiquitin from E1 to the active site cysteine of the E2 via a trans (thio) esterification reaction. The E2 binds to both the activated ubiquitin and the E1 enzyme. Humans have 35 different E2 enzymes, characterized by their highly conserved structure, which is known as the ubiquitin-conjugating catalytic (UBC) fold. The E3 ubiquitin ligases facilitate the final step of the ubiquitination cascade. Generally, they create an isopeptide bond between a lysine of the target protein and the C-terminal glycine of ubiquitin. There are hundreds of E3 ligases; some also activate the E2 enzymes. E3 enzymes function as the substrate recognition modules of the system and interact with both the E2 and the substrate. The enzymes possess one of two domains: the homologous to the E6-AP carboxyl terminus (HECT) domain or the really interesting new gene (RING) domain (or the closely related, U-box domain). HECT domain E3 enzymes transiently bind ubiquitin when an obligate thioester intermediate is formed with the active-site cysteine of the E3, whereas RING domain E3 enzymes catalyze the direct transfer from the E2 enzyme to the substrate.

The number of ubiquitins added to the antigen can enhance the efficacy of the processing step. For instance, in polyubiquitination, additional ubiquitin molecules are added after the first has been attached to the peptide. The resulting ubiquitin chain is created by the linking of the glycine residue of the ubiquitin molecule to a lysine of the ubiquitin bound to the peptide. Each ubiquitin contains seven lysine residues and an N-terminal that can serve as sites for ubiquitination. When four or more ubiquitin molecules are attached to a lysine residue on the peptide antigen, the 26S proteasome recognizes the complex, internalizes it, and degrades the protein into small peptides.

Ubiquitin wild type has the following sequence (Homo sapiens):

(SEQ ID NO: 1318)
MQIFVKTLTGKTITLEVEPSDTIENVKAKIQDKEGIPPDQQRLIFAGKQLE
DGRTLSDYNIQKESTLHLVLRLRGG

The epitopes are connected in some embodiments by a cleavage sensitive site. A cleavage sensitive site is a peptide which is susceptible to cleavage by an enzyme or protease. These sites are also called protease cleavage sites. In some embodiments the protease is an intracellular enzyme. In some embodiments the protease is a protease found in an Antigen Presenting Cell (APC). Thus, protease cleavage sites correspond to high abundance (highly expressed) proteases in APCs. A cleavage sensitive site that is sensitive to an APC enzyme is referred to as an APC cleavage sensitive site. Proteases expressed in APCs include but are not limited to Cysteine proteases, such as: Cathepsin B, Cathepsin H, Cathepsin L, Cathepsin S, Cathepsin F, Cathepsin Z, Cathepsin V, Cathepsin O, Cathepsin C, and Cathepsin K, and Aspartic proteases such as Cathepsin D, Cathepsin E, and Asparaginyl endopeptidase.

The following are exemplary APC cleavage sensitive sites:
Cathepsin B: cleavage on the caboxyl side of Arg-Arg bonds
Cathepsin D has the following preferential cleavage sequences:

| P6 | P5 | P4 | P3 | P2 | P1 | ↓ | P1′ | P2′ | P3′ | P4′ |
|---|---|---|---|---|---|---|---|---|---|---|
| Xaa | Xaa | Xaa | Xaa | hydro | hydro | ↓ | hydro | Xaa | Xaa | Xaa |
| Xaa | Xaa | Xaa | Xaa | Glu | hydro | ↓ | hydro | Xaa | Xaa | Xaa, |

where Xaa=any amino acid residue, hydro=Ala, Val, Leu, Ile, Phe, Trp, or Tyr, and ↓=cleavage site
Cathepsin H: Arg-↓-NHMec; Bz-Arg-↓-NhNap; Bz-Arg-↓NHMec; Bz-Phe-Cal-Arg-↓-NHMec; Pro-Gly-↓-Phe
Cathepsin S and F: Xaa-Xaa-Val-Val-Arg-Xaa-Xaa
where Xaa=any amino acid residue
Cathepsin V: Z-Phe-Arg-NHMec; Z-Leu-Arg-NHMec; Z-Val-Arg-NHMec
Cathepsin O: Z-Phe-Arg-NHMec and Z-Arg-Arg-NHMec
Cathepsin C has the following preferential cleavage sequences:

| P2 | P1 | ↓ | P1′ | P2′ | P3′ | P4′ |
|---|---|---|---|---|---|---|
| not Arg | not Pro | ↓ | not Pro | Xaa | Xaa | Xaa |
| not Lys | not Pro | ↓ | not Pro | Xaa | Xaa | Xaa |

where Xaa=any amino acid residue and ↓=cleavage site
Cathepsin E: Arg-X, Glu-X, and Arg-Arg
Asparaginyl endopeptidase: after asparagine residues
Cathepsin L has the following preferential cleavage sequences:

| | P6 | P5 | P4 | P3 | P2 P1 | ↓ | P1′ | P2′ | P3′ | P4′ |
|---|---|---|---|---|---|---|---|---|---|---|
| | | Xaa | Xaa | Xaa | hydrophobic | Phe | Arg | ↓ | Xaa | Xaa |
| Xaa | Xaa | | | | | | | | | |
| | | Xaa | Xaa | Xaa | aromatic | Phe | Arg | ↓ | Xaa | Xaa |
| Xaa | Xaa | | | | | | | | | |
| | | Xaa | Xaa | Xaa | hydrophobic | Arg | Arg | ↓ | Xaa | Xaa |
| Xaa | Xaa | | | | | | | | | |
| | | Xaa | Xaa | Xaa | aromatic | Arg | Arg | ↓ | Xaa | Xaa |
| Xaa | Xaa, | | | | | | | | | |

where Xaa=any amino acid residue, hydrophobic=Ala, Val, Leu, Ile, Phe, Trp, or Tyr, aromatic=Phe, Trp, His, or Tyr, and ↓=cleavage site

In some embodiments the cleavage sensitive site is a cathepsin B or S sensitive sites. Exemplary cathepsin B sensitive sites include, but are not limited to, those set forth in SEQ ID Nos: 226-615. Exemplary cathepsin S sensitive sites include, but are not limited to, those set forth in SEQ ID Nos: 616-1313.

In some embodiments, the mRNA cancer vaccines and vaccination methods include an mRNA encoding a concatemeric cancer antigen comprised of one or more neoepitopes and one or more traditional, cancer antigens. In some embodiments, the mRNA encodes 1, 2, 3, 4, 5, 6, 7, 8, 9, 10, 11, 12, 13, 14, 15, 16, 17, 18, 19, 20, or more traditional, cancer antigens in addition to the encoded neoepitopes.

In some embodiments the concatemeric antigen encodes 5-10 cancer peptide epitopes. In yet other embodiments the concatemeric antigen encodes 25-100 cancer peptide epitopes. In some embodiments the mRNA cancer vaccines and vaccination methods include epitopes or antigens based on specific mutations (neoepitopes) and those expressed by cancer-germline genes (antigens common to tumors found in multiple patients). In some embodiments, the mRNA cancer vaccines and vaccination methods include one or more traditional epitopes or antigens, e.g., one or more epitopes or antigens found in a traditional cancer vaccine.

The neoepitopes selected for inclusion in the concatemeric antigen typically will be high affinity binding peptides. The neoepitopes in the concatemeric construct may be the same or different, e.g., they vary by length, amino acid sequence or both.

In some embodiments, the neoepitopes are interspersed by linkers.

In some embodiments, the vaccine may be a polycistronic vaccine including multiple neoepitopes or one or more single mRNA vaccines or a combination thereof.

In some embodiments, the mRNA bacterial vaccines and vaccination methods include an mRNA encoding a concatemeric bacterial antigen comprised of one or more bacterial antigens. In some embodiments, the mRNA encodes 1, 2, 3, 4, 5, 6, 7, 8, 9, 10, 11, 12, 13, 14, 15, 16, 17, 18, 19, 20, or more bacterial antigens.

Compositions of Immune Potentiator mRNAs and Antigens of Interest

In another aspect, the disclosure provides a composition comprising at least one chemically modified messenger RNA (mmRNA) encoding:(i) at least one antigen of interest; and (ii) at least one polypeptide that enhances an immune response against the at least one antigen of interest when the at least on mmRNA is administered to a subject, wherein said mmRNA comprises one or more modified nucleobases. Thus, the disclosure provides compositions comprising at least one immune potentiator mRNA and at least one mRNA encoding an antigen of interest, wherein a single mRNA construct can encode both the antigen(s) or interest and the polypeptide that enhances an immune response to the antigen(s) or, alternatively, the composition can comprise two or more separate mRNA constructs, a first mRNA and a second mRNA, wherein the first mRNA encodes the at least one antigen of interest and the second mRNA encodes the polypeptide that enhances an immune response to the antigen(s) (i.e., the second mRNA comprises the immune potentiator).

In those embodiments comprising a first mRNA encoding an antigen(s) of interest and a second mRNA encoding the polypeptide that enhances an immune response to the antigen(s) of interest, the first mRNA and the second mRNAs can be coformulated together (e.g., prior to coadministration), such as coformulated in the same lipid nanoparticle.

In those embodiments comprising a single mRNA encoding both the antigen(s) of interest and the polypeptide that enhances an immune response to the antigen(s) of interest, the sequences encoding the polypeptide can be positioned on the mRNA construct either upstream or downstream of the sequences encoding the antigen of interest. For example, non-limiting examples of mRNA constructs encoding both an antigen and an immunostimulatory polypeptide include those encoding at least one mutant KRAS antigen and a constitutively active STING polypeptide, e.g., encoding an amino acid sequence shown in any one of SEQ ID NOs: 107-130. In one embodiment, the constitutively active STING polypeptide is located at the N-terminal end of the construct (i.e., upstream of the antigen-encoding sequences), as shown in SEQ ID NOs: 107-118. In another embodiment, the constitutively active STING polypeptide is located at the C-terminal end of the construct (i.e., downstream of the antigen-encoding sequences), as shown in SEQ ID NOs: 119-130.

Various mRNAs encoding antigens of interest (e.g., mRNA vaccines) that can be used in combination with an immune potentiator mRNA of the disclosure are described in further detail below.

Immunogenic Cell Death-Inducing mRNA Constructs

In another aspect, the disclosure provides mRNA constructs (e.g., mmRNAs) encoding polypeptides that induce immunogenic cell death, such as necroptosis or pyroptosis. The immunogenic cell death induced by the mRNAs results in release of cytosolic components from the cell such that an immune response against the cell is stimulated in vivo. Thus, the mRNAs of the invention can be used to stimulate an immune response in vivo against cells of interest, such as tumors in the treatment of cancer. An mRNA encoding a polypeptide that induces immunogenic cell death can be used alone or, alternatively, can be used in combination with one or more additional agents that stimulate or enhance immune responsiveness. Such additional agents include agents that stimulate adaptive immunity, such as stimulation of Type I interferon production, agents that induce T cell activation or priming and/or agents that modulate one or more immune checkpoints. Such additional agents can also be mRNAs or, alternatively, can be a different type of agent, such as a protein, antibody or small molecule. In one embodiment, the additional agent is one or more immune potentiator mRNA constructs of the disclosure.

Immunogenic cell death is distinguishable from non-immunogenic cell death in that immunogenic cell death results in release of intracellular components from the cell into the surrounding environment such that those components are made available for stimulation of an immune response. A number of intracellular components have been identified that typically are released during immunogenic cell death, referred to as “damage-associated molecular patterns” or DAMPs, including ATP, HMGB1, IL-1a, uric acid, DNA fragments, histones and mitochondrial content. DAMPs may be released extracellularly or certain DAMPs are translocated from the interior of the cell to the cell surface (e.g., calreticulin, which translocates from the lumen of the endoplasmic reticulum to the cell surface). Thus, release of DAMPs serves as an indicator of immunogenic cell death. Immunogenic cell death is also characterized by stimulation of pro-inflammatory cytokines.

Two types of immunogenic cell death are necroptosis and pyroptosis. Each of these types of programmed cell death has characteristic features that distinguish them from each other and from apoptosis, which is a form of programmed non-immunogenic cell death. Distinguishing characteristics of apoptosis are that it is caspase-dependent (e.g., dependent on initiator caspases such as caspase-8 and -10 for death receptor-induced apoptosis or caspase-9 for intrinsically-triggered apoptosis) and leads to cytoplasmic concentration and cell shrinkage, plasma membrane blebbing (but not loss of plasma membrane integrity), increased intracellular calcium concentration and mitochondrial outer membrane permeabilization (MOMP). Importantly, apoptosis does not result in release of intracellular components into the surrounding environment and is considered to be immunologically tolerogenic. In contrast, necroptosis is not dependent on caspase activity but is dependent on the activity of a kinase, referred to as Receptor Interacting Protein Kinase 1 (RIPK1). In fact, activation of caspases inhibits necroptosis, since, for example, activated caspase-8 and -10 inactivate RIPK1. When RIPK1 is activated, it interacts with RIPK3, leading to formation of the necrosome complex. Cell death by necroptosis is also dependent on Mixed Lineage Kinase Domain-Like protein (MLKL). Necroptosis is characterized by cellular collapse and loss of plasma membrane integrity, including release of DAMPs. Pyroptosis is also characterized by release of DAMPs, but differs from necroptosis in that it is dependent on gasdermin D (GSDMD), NLR family pyrin domain containing-3 (NLRP3; encodes crypyrin) and caspase 1, as well as caspase-4 and caspase-5 in humans and caspase-11 in mice, leading to induction of the inflammasome. Additional forms of caspase-independent immunogenic cell death that lead to plasma membrane rupture and inflammation include mitochondrial permeability transition-mediated regulated necrosis (MPT-RN), ferroptosis, parthanatos and NETosis (for review, see e.g., Linkermann, A. et al. (2014) Nat. Rev. Immunol. 14:759-767).

In one embodiment, the invention provides an mRNA encoding a polypeptide that induces necroptosis. In another embodiment, the invention provides an mRNA encoding a polypeptide that induces pyroptosis. In yet other embodiments, the invention provides an mRNA encoding a polypeptide that induces MPT-RN, ferroptosis, parthanatos or NETosis.

In one embodiment, the polypeptide that induces necroptosis is mixed lineage kinase domain-like protein (MLKL), or an immunogenic cell death-inducing fragment thereof. As described further in Examples 22-23, MLKL constructs induce necroptotic cell death, characterized by release of DAMPs. In one embodiment, the mRNA construct encodes amino acids 1-180 of human or mouse MLKL. In one embodiment, the MLKL construct comprises one or more miR binding sites. In one embodiment, the MLKL construct comprises a miR 122 binding site, a miR142-3p binding site or both binding sites, for example in the 3′ UTR or in the 5′ UTR. Non-limiting examples of mRNA constructs encoding MLKL, or an immunogenic cell death-inducing fragment thereof, encode amino acids 1-180 of human or mouse MLKL comprising the amino sequences shown in SEQ ID NOs: 1327 and 1328, respectively.

In another embodiment, the polypeptide is receptor-interacting protein kinase 3 (RIPK3), or an immunogenic cell death-inducing fragment thereof. As described further in Example 24, RIPK3 constructs induce necroptotic cell death. In one embodiment, the mRNA construct encodes a RIPK3 polypeptide that multimerize with itself (homo-oligomerization). In one embodiment, the mRNA construct encodes a RIPK3 polypeptide that dimerizes with RIPK1. In one embodiment, the mRNA construct encodes the kinase domain and the RHIM domain of RIPK3. In one embodiment, the mRNA construct encodes the kinase domain of RIPK3, the RHIM domain of RIPK3 and two FKBP (F>V) domains. In one embodiment, the mRNA construct encodes a RIPK3 polypeptide (e.g., comprising the kinase domain and the RHIM domain of RIPK3) and an IZ domain (e.g., an IZ trimer). In one embodiment, the mRNA construct encodes a RIPK3 polypeptide (e.g., comprising the kinase domain and the RHIM domain of RIPK3) and one or more EE or RR domains (e.g., 2xEE domains, or 2xRR domains). Additionally, the structure of DNA constructs encoding RIPK3 constructs that induce immunogenic cell death are described further in, for example, Yatim, N. et al. (2015) Science 350:328-334 or Orozco, S. et al. (2014) Cell Death Differ. 21:1511-1521, and can be used in the design of suitable RNA constructs. In one embodiment, the RIPK3 construct comprises one or more miR binding sites. In one embodiment, the RIPK3 construct comprises a miR122 binding site, a miR142-3p binding site or both binding sites, e.g., in the 3′ UTR or the 5′ UTR. Non-limiting examples of mRNA constructs encoding RIPK3, or an immunogenic cell death-inducing fragment thereof, comprise an ORF having any of the amino acid sequences shown in SEQ ID NOs: 1329-1344.

In another embodiment, the polypeptide is receptor-interacting protein kinase 1 (RIPK1), or an immunogenic cell death-inducing fragment thereof. In one embodiment, the mRNA construct encodes amino acids 1-155 of a human or mouse RIPK1 polypeptide. In another embodiment, the mRNA construct encodes a RIPK1 polypeptide and an IZ domain. In another embodiment, the mRNA construct encodes a RIPK1 polypeptide and a DM domain. In one embodiment, the mRNA construct encodes a RIPK1 polypeptide and one or more EE or RR domains. Additionally, the structure of DNA constructs encoding RIPK1 constructs that induce immunogenic cell death are described further in, for example, Yatim, N. et al. (2015) Science 350:328-334 or Orozco, S. et al. (2014) Cell Death Differ. 21:1511-1521, and can be used in the design of suitable RNA constructs. In one embodiment, the RIPK1 construct comprises one or more miR binding sites. In one embodiment, the RIPK1 construct comprises a miR122 binding site, a miR142-3p binding site or both binding sites, e.g., in the 3′ UTR or in the 5′ UTR. Non-limiting examples of mRNA constructs encoding RIPK1, or an immunogenic cell death-inducing fragment thereof, comprise an ORF having any of the amino acid sequences shown in SEQ ID NOs: 158-163.

In another embodiment, the polypeptide is direct IAP binding protein with low pI (DIABLO) (also known as SMAC/DIABLO), or an immunogenic cell death-inducing fragment thereof. As described in the examples, DIABLO constructs induce cell death and release of cytokines. In one embodiment, the mRNA construct encodes a wild-type human DIABLO Isoform 1 sequence. In another embodiment, the mRNA construct encodes a human DIABLO Isoform 1 sequence comprising an S126L mutation. In another embodiment, the mRNA construct encodes amino acids 56-239 of human DIABLO Isoform 1. In another embodiment, the mRNA construct encodes amino acids 56-239 of human DIABLO Isoform 1 and comprises an S126L mutation. In another embodiment, the mRNA construct encodes a wild-type human DIABLO Isoform 3 sequence. In another embodiment, the mRNA construct encodes a human DIABLO Isoform 3 sequence comprising an S27L mutation. In another embodiment, the mRNA construct encodes amino acids 56-240 of human DIABLO Isoform 3. In another embodiment, the mRNA construct encodes amino acids 56-240 of human DIABLO Isoform 3 and comprises an S27L mutation. In one embodiment, the DIABLO construct comprises one or more miR binding sites. In one embodiment, the DIABLO construct comprises a miR122 binding site, a miR142-3p binding site or both binding sites, e.g., in the 3′ UTR or in the 5′ UTR. Non-limiting examples of mRNA constructs encoding DIABLO, or an immunogenic cell death-inducing fragment thereof, comprise an ORF having any of the amino acid sequences shown in SEQ ID NOs: 165-172.

In another embodiment, the polypeptide is FADD (Fas-associated protein with death domain), or an immunogenic cell death-inducing fragment thereof. In one embodiment, the FADD construct comprises one or more miR binding sites. In one embodiment, the FADD construct comprises a miR122 binding site, a miR142-3p binding site or both binding sites, e.g. in the 3′ UTR or in the 5′ UTR. Non-limiting examples of mRNA constructs encoding FADD, or an immunogenic cell death-inducing fragment thereof, comprise and ORF having any of the amino acid sequences shown in SEQ ID NOs: 1345-1351.

In another embodiment, the invention provides an mRNA encoding a polypeptide that induces pyroptosis. In one embodiment, the polypeptide is gasdermin D (GSDMD), or an immunogenic cell death-inducing fragment thereof. In one embodiment, the mRNA construct encodes a wild-type human GSDMD sequence. In another embodiment, the mRNA construct encodes amino acids 1-275 of human GSDMD. In another embodiment, the mRNA construct encodes amino acids 276-484 of human GSDMD. In another embodiment, the mRNA construct encodes wild-type mouse GSDMD. In another embodiment, the mRNA construct encodes amino acids 1-276 of mouse GSDMD. In another embodiment, the mRNA construct encodes amino acids 277-487 of mouse GSDMD. In one embodiment, the GSDMD construct comprises one or more miR binding sites. In one embodiment, the GSDMD construct comprises a miR122 binding site, a miR142-3p binding site or both binding sites, e.g., in the 3′ UTR or in the 5′ UTR. Non-limiting examples of mRNA constructs encoding GSDMD, or an immunogenic cell-death inducing fragment thereof, encode any of the amino acid sequences shown in SEQ ID NOs: 1367-1372.

In another embodiment, the polypeptide is caspase-4 or caspase-5 or caspase-11, or an immunogenic cell death-inducing fragment thereof. In various embodiments, the caspase-4, -5 or -11 construct can encode (i) full-length wild-type caspase-4, caspase-5 or caspase-11; (ii) full-length caspase-4, -5 or -11 plus an IZ domain; (iii) N-terminally deleted caspase-4, -5 or -11 plus an IZ domain; (iv) full-length caspase-4, -5 or -11 plus a DM domain; or (v) N-terminally deleted caspase-4, -5 or -11 plus a DM domain. Examples of N-terminally deleted forms of caspase-4 and caspase-11 contain amino acid residues 81-377. An example of an N-terminally deleted form of caspase-5 contains amino acid residues 137-434. In one embodiment, the caspase-4, -5 or -11 construct comprises one or more miR binding sites. In one embodiment, the caspase-4, -5 or -11 construct comprises a miR122 binding site, a miR 142-3p binding site or both binding sites, e.g., in the 3′ UTR or in the 5′ UTR. Non-limiting examples of mRNA constructs encoding caspase-4, or an immunogenic cell death-inducing fragment thereof, comprise an ORF having any of the amino acid sequences shown in SEQ ID NOs: 1352-1356. Non-limiting examples of mRNA constructs encoding caspase-5, or an immunogenic cell death-inducing fragment thereof, comprise an ORF having any of the amino acid sequences shown in SEQ ID NOs: 1357-1361. Non-limiting examples of mRNA constructs encoding caspase-11, or an immunogenic cell death-inducing fragment thereof, comprise an ORF having any of the amino acid sequences shown in SEQ ID NOs: 1362-1366.

In another embodiment, the polypeptide is NLRP3, or an immunogenic cell death-inducing fragment thereof. In one embodiment, the NLRP3 construct comprises one or more miR binding sites. In one embodiment, the NLRP3 construct comprises a miR122 binding site, a miR142-3p binding site or both binding sites, e.g., in the 3′ UTR or the 5′ UTR. Non-limiting examples of mRNA constructs encoding NLRP3, or an immunogenic cell death-inducing fragment thereof, encode the ORF amino acid sequences shown in SEQ ID NOs: 1373 or 1374.

In another embodiment, the polypeptide is apoptosis-associated speck-like protein containing a CARD (ASC/PYCARD), or an immunogenic cell death-inducing fragment thereof, such as a Pyrin domain. In one embodiment, the polypeptide is a Pyrin B30.2 domain. In another embodiment, the polypeptide is a Pyrin B30.2 domain comprising a V726A mutation. In one embodiment, the ASC/PYCARD or Pyrin construct comprises one or more miR binding sites. In one embodiment, the ASC/PYCARD or Pyrin construct comprises a miR122 binding site, a miR142-3p binding site or both binding sites, e.g., in the 3′ UTR or in the 5′ UTR. Non-limiting examples of mRNA constructs encoding a Pyrin B30.2 domain encode the ORF amino acid sequences shown in SEQ ID NOs: 1375 or 1376. Non-limiting examples of mRNA constructs encoding ASC encode the ORF amino acid sequences shown in SEQ ID NOs: 1377 or 1378.

The mRNAs of the invention encoding a polypeptide that induces immunogenic cell death can be used in combination with other agents that stimulate an immflammatory and/or immune reaction and/or regulate immunoresponsiveness. For an immune response against cancer cells to be effective in killing of the cancer cells, a number of events have been described that must occur in a stepwise fashion and be allowed to proceed and expand iteratively. This process has been referred to as the Cancer-Immunity Cycle (see e.g., Chen, D. S. and Mellman, I. (2013) Immunity, 39:1-10). These sequential events involve: (i) release of cancer cell antigens; (ii) cancer antigen presentation (e.g., by dendritic cells or other antigen presenting cells); (iii) priming and activation of T cells; (iv) trafficking of T cells (e.g., CTLs) to the tumor; (v) infiltration of T cells into the tumor; (vi) recognition of cancer cells by the T cells; and (vii) killing of the cancer cells.

Accordingly, another aspect of the invention pertains to additional agents that can be used in combination with an mRNA of the invention encoding a polypeptide that induces immunogenic cell death in order promote or enhance an immune response against cellular antigens of the cell targeted for killing. Such additional agents may stimulate or promote an inflammatory and/or immune response. Additionally or alternatively, such additional agents may regulate immune responsiveness, for example by acting as an immune checkpoint modulator. An additional agent can also be an mRNA, e.g., having structural properties as described herein for mRNA constructs (e.g., modified nucleobases, 5′ cap, 5′ UTR, 3′ UTR, miR binding site(s), polyA tail, as described herein). Alternatively, an additional agent can be a non-mRNA agent, such as a protein, antibody or small molecule.

In one embodiment, the additional agent potentiates an immune response, for example, induces adaptive immunity (e.g., by stimulating Type I interferon production), stimulates an inflammatory response, stimulates NFκB signaling and/or stimulates dendritic cell (DC) mobilization. In one embodiment, the agent that induces adaptive immunity is Type I interferon. For example, a pharmaceutical composition comprising Type I interferon can be used as the agent. Alternatively, in another embodiment, the additional agent that induces adaptive immunity is an agent that stimulates Type I interferon production. Non-limiting examples of agents that stimulate Type I interferon production include STING, IRF1, IRF3, IRF5, IRF6, IRF7 and IRF8. Non-limiting examples of agents that stimulate an inflammatory response include STAT1, STAT2, STAT4, STAT6, NFAT and C/EBPb. Non-limiting examples of agents that stimulate NFκB signaling include IKKβ, c-FLIP, RIPK1, IL-27, ApoF and PLP. A non-limiting example of an agent that stimulates DC mobilization is FLT3. Yet another agent that potentiates immune response is DIABLO (SMAC/DIABLO).

In one embodiment, the agent that potentiates an immune response is an immune potentiator mRNA construct of the disclosure, non-limiting examples of which include constructs encoding STING, IRF3, IRF7, STAT6, Myd88, Btk (E41K), TAK-TAB1, DIABLO (SMAC/DIABLO), TRAM (TICAM2) polypeptide or a self-activating caspase-1 polypeptide, constitutively active IKKβ, constitutively active IKKα, c-FLIP and RIPK1 mRNA constructs.

In another embodiment, the additional agent induces T cell activation or priming. For example, the additional agent that induces T cell activation or priming can be a cytokine or a chemokine. Non-limiting examples of cytokines or chemokines that induce T cell activation or priming include IL-12, IL36g, CCL2, CCL4, CCL20 and CCL21. In one embodiment, the agent is a pharmaceutical composition that comprises the cytokine or chemokine. In another embodiment, the agent is one that induces production of the cytokine or chemokine. In another embodiment the agent is an mRNA construct encoding the cytokine or chemokine. In another embodiment, the agent is an mRNA construct encoding a polypeptide that induces the chemokine or cytokine.

In another embodiment, the additional agent modulates an immune checkpoint. Various immune checkpoint inhibitors have been described in the art, including PD-1 inhibitors, PD-L1 inhibitors and CTLA-4 inhibitors. Other modulators of immune checkpoints may target OX-40, OX-40L or ICOS. In one embodiment, an agent that modulates an immune checkpoint is an antibody. In another embodiment, an agent that modulates an immune checkpoint is a protein or small molecule modulator. In another embodiment, the agent (such as an mRNA) encodes an antibody modulator of an immune checkpoint.

In one embodiment, the additional agent that modulates an immune checkpoint targets PD-1. Non-limiting examples of immunotherapeutic agents that target PD-1 include pembrolizumab, alemtuzumab, atezolizumab, nivolumab, ipilimumab, pidilizumab, ofatumumab, rituximab, MEDI0680 and PDR001, AMP-224, PF-06801591, BGB-A317, REGN2810, SHR-1210, TSR-042, avelumab, durvalumab and affimer.

In one embodiment, the additional agent that modulates an immune checkpoint targets PD-L1. Non-limiting examples of immunotherapeutic agents that target PD-L1 include avelumab (MSB0010718C), atezolizumab (MPDL3280A), durvalumab (MEDI4736) and BMS936559.

In one embodiment, the additional agent that modulates an immune checkpoint targets CTLA-4. Non-limiting examples of immunotherapeutic agents that target CTLA-4 include ipilimumab, tremelimumab and AGEN1884.

In one embodiment, the additional agent that modulates an immune checkpoint targets OX-40 or OX-40L. In one embodiment, the agent that targets OX-40 or OX-40L is an mRNA construct encoding an Fc-OX-40L polypeptide. In yet other embodiments, the agent that targets OX-40 or OX-40L is an immunostimulatory agonist anti-OX-40 or OX-40L antibody, examples of which known in the art include MEDI6469 (agonist anti-OX40 antibody) and MOXR0916 (agonist anti-OX40 antibody).

In yet another embodiment, the additional agent that modulates an immune checkpoint is an ICOS pathway agonist.

mRNA Construct Components

An mRNA may be a naturally or non-naturally occurring mRNA. An mRNA may include one or more modified nucleobases, nucleosides, or nucleotides, as described below, in which case it may be referred to as a “modified mRNA” or “mmRNA.” As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID NO: 21. Another exemplary 5′ UTR for use in the constructs is shown in SEQ ID NO: 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G (5′)ppp(5′) G, commonly written as m⁷GpppG. A cap species may also be an anti-reverse cap analog. A non-limiting list of possible cap species includes m⁷GpppG, m⁷Gpppm7G, m⁷3′dGpppG, m₂^{7,O3}′GpppG, m₂^{7,O3}′GppppG, m₂^{7,O2}′GppppG, m⁷Gpppm⁷G, m⁷3′dGpppG, m₂^{7,O3}′GpppG, m₂^{7,O3}′GppppG, and m₂^{7,O2}′GppppG.

An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA.

An mRNA may instead or additionally include a microRNA binding site.

In some embodiments, an mRNA is a bicistronic mRNA comprising a first coding region and a second coding region with an intervening sequence comprising an internal ribosome entry site (IRES) sequence that allows for internal translation initiation between the first and second coding regions, or with an intervening sequence encoding a self-cleaving peptide, such as a 2A peptide. IRES sequences and 2A peptides are typically used to enhance expression of multiple proteins from the same vector. A variety of IRES sequences are known and available in the art and may be used, including, e.g., the encephalomyocarditis virus IRES.

In one embodiment, the polynucleotides of the present disclosure may include a sequence encoding a self-cleaving peptide. The self-cleaving peptide may be, but is not limited to, a 2A peptide. A variety of 2A peptides are known and available in the art and may be used, including e.g., the foot and mouth disease virus (FMDV) 2A peptide, the equine rhinitis A virus 2A peptide, the Thosea asigna virus 2A peptide, and the porcine teschovirus-1 2A peptide. 2A peptides are used by several viruses to generate two proteins from one transcript by ribosome-skipping, such that a normal peptide bond is impaired at the 2A peptide sequence, resulting in two discontinuous proteins being produced from one translation event. As a non-limiting example, the 2A peptide may have the protein sequence: GSGATNFSLLKQAGDVEENPGP (SEQ ID NO: 24), fragments or variants thereof. In one embodiment, the 2A peptide cleaves between the last glycine and last proline. As another non-limiting example, the polynucleotides of the present disclosure may include a polynucleotide sequence encoding the 2A peptide having the protein sequence GSGATNFSLLKQAGDVEENPGP (SEQ ID NO: 24) fragments or variants thereof. One example of a polynucleotide sequence encoding the 2A peptide is: GGAAGCGGAGCTACTAACTTCAGCCTGCTGAAGCAGGCTGGAGACGTGGAGGAG AACCCTGGACCT (SEQ ID NO: 25). In one illustrative embodiment, a 2A peptide is encoded by the following sequence: 5′-TCCGGACTCAGATCCGGGGATCTCAAAATTGTCGCTCCTGTCAAACAAACTCTTA ACTTTGATTTACTCAAACTGGCTGGGGATGTAGAAAGCAATCCAGGTCCACTC-3′ (SEQ ID NO: 26). The polynucleotide sequence of the 2A peptide may be modified or codon optimized by the methods described herein and/or are known in the art.

In one embodiment, this sequence may be used to separate the coding regions of two or more polypeptides of interest. As a non-limiting example, the sequence encoding the F2A peptide may be between a first coding region A and a second coding region B (A-F2Apep-B). The presence of the F2A peptide results in the cleavage of the one long protein between the glycine and the proline at the end of the F2A peptide sequence (NPGP is cleaved to result in NPG and P) thus creating separate protein A (with 21 amino acids of the F2A peptide attached, ending with NPG) and separate protein B (with 1 amino acid, P, of the F2A peptide attached). Likewise, for other 2A peptides (P2A, T2A and E2A), the presence of the peptide in a long protein results in cleavage between the glycine and proline at the end of the 2A peptide sequence (NPGP is cleaved to result in NPG and P). Protein A and protein B may be the same or different peptides or polypeptides of interest. In particular embodiments, protein A is a polypeptide that induces immunogenic cell death and protein B is another polypeptide that stimulates an inflammatory and/or immune response and/or regulates immune responsiveness (as described further below).

Modified mRNAs

While in certain embodiments an mRNA of the disclosure entirely comprises unmodified nucleobases, nucleosides or nucleotides, in some embodiments, an mRNA of the disclosure comprises one or more modified nucleobases, nucleosides, or nucleotides (termed “modified mRNAs” or “mmRNAs”). In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (w), pyridin-4-one ribonucleoside, 5-aza-uridine, 6-aza-uridine, 2-thio-5-aza-uridine, 2-thio-uridine (s²U), 4-thio-uridine (s4U), 4-thio-pseudouridine, 2-thio-pseudouridine, 5-hydroxy-uridine (ho⁵U), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (mcmo⁵U), 5-carboxymethyl-uridine (cm⁵U), 1-carboxymethyl-pseudouridine, 5-carboxyhydroxymethyl-uridine (chm⁵U), 5-carboxyhydroxymethyl-uridine methyl ester (mchm⁵U), 5-methoxycarbonylmethyl-uridine (mcm⁵U), 5-methoxycarbonylmethyl-2-thio-uridine (mcm⁵s²U), 5-aminomethyl-2-thio-uridine (nm⁵s²U), 5-methylaminomethyl-uridine (mnm⁵U), 5-methylaminomethyl-2-thio-uridine (mnm⁵s²U), 5-methylaminomethyl-2-seleno-uridine (mnm⁵se²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm⁵U), 1-taurinomethyl-pseudouridine, 5-taurinomethyl-2-thio-uridine (m⁵s²U), 1-taurinomethyl-4-thio-pseudouridine, 5-methyl-uridine (m⁵U, i.e., having the nucleobase deoxythymine), 1-methyl-pseudouridine (ml), 5-methyl-2-thio-uridine (m⁵s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m⁵D), 2-thio-dihydrouridine, 2-thio-dihydropseudouridine, 2-methoxy-uridine, 2-methoxy-4-thio-uridine, 4-methoxy-pseudouridine, 4-methoxy-2-thio-pseudouridine, N1-methyl-pseudouridine, 3-(3-amino-3-carboxypropyl) uridine (acp³U), 1-methyl-3-(3-amino-3-carboxypropyl) pseudouridine (acp³ ψ), 5-(isopentenylaminomethyl) uridine (inm⁵U), 5-(isopentenylaminomethyl)-2-thio-uridine (inm⁵s²U), α-thio-uridine, 2′-O-methyl-uridine (Um), 5,2′-O-dimethyl-uridine (m⁵Um), 2′-O-methyl-pseudouridine (vm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)]uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m⁵Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴₂Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include «-thio-adenosine, 2-amino-purine, 2,6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino-purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms²m⁶A), N⁶-isopentenyl-adenosine (i⁶A), 2-methylthio-N6-isopentenyl-adenosine (ms²i⁶A), N6-(cis-hydroxyisopentenyl) adenosine (io⁶A), 2-methylthio-N6-(cis-hydroxyisopentenyl) adenosine (ms²io⁶A), N6-glycinylcarbamoyl-adenosine (g⁶A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6,N6-dimethyl-adenosine (m⁶₂A), N6-hydroxynorvalylcarbamoyl-adenosine (hn⁶A), 2-methylthio-N6-hydroxynorvalylcarbamoyl-adenosine (ms²hn⁶A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6,N6,2′-O-trimethyl-adenosine (m⁶²Am), 1,2′—O-dimethyl-adenosine (m¹Am), 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara-adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (I), 1-methyl-inosine (m¹I), wyosine (imG), methylwyosine (mimG), 4-demethyl-wyosine (imG-14), isowyosine (imG2), wybutosine (yW), peroxywybutosine (02yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQ₀), 7-aminomethyl-7-deaza-guanosine (preQ₁), archaeosine (G⁺), 7-deaza-8-aza-guanosine, 6-thio-guanosine, 6-thio-7-deaza-guanosine, 6-thio-7-deaza-8-aza-guanosine, 7-methyl-guanosine (m⁷G), 6-thio-7-methyl-guanosine, 7-methyl-inosine, 6-methoxy-guanosine, 1-methyl-guanosine (m¹G), N2-methyl-guanosine (m²G), N2,N2-dimethyl-guanosine (m²²G), N2,7-dimethyl-guanosine (m^{2,7}G), N2, N2,7-dimethyl-guanosine (m^{2,2,7}G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²²Gm), 1-methyl-2′-O-methyl-guanosine (m¹Gm), N2,7-dimethyl-2′-O-methyl-guanosine (m^{2,7}Gm), 2′-O-methyl-inosine (Im), 1,2′-O-dimethyl-inosine (m¹Im), 2′-O-ribosylguanosine (phosphate) (Gr (p)), 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is pseudouridine (w), N1-methylpseudouridine (mw), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases). In some embodiments, the modified nucleobase is N1-methylpseudouridine (m¹w) and the mRNA of the disclosure is fully modified with N1-methylpseudouridine (m¹w). In some embodiments, N1-methylpseudouridine (m¹w) represents from 75-100% of the uracils in the mRNA. In some embodiments, N1-methylpseudouridine (m¹w) represents 100% of the uracils in the mRNA.

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²° C.), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁶A). In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQ₀), 7-aminomethyl-7-deaza-guanosine (preQ₁), 7-methyl-guanosine (m⁷G), 1-methyl-guanosine (m¹G), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases).

In some embodiments, the modified nucleobase is 1-methyl-pseudouridine (m¹w), 5-methoxy-uridine (mo⁵U), 5-methyl-cytidine (m⁵C), pseudouridine (w), α-thio-guanosine, or α-thio-adenosine. In some embodiments, an mRNA of the disclosure includes a combination of one or more of the aforementioned modified nucleobases (e.g., a combination of 2, 3 or 4 of the aforementioned modified nucleobases).

In some embodiments, the mRNA comprises pseudouridine (ψ). In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (mo⁵U) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises comprises N6-methyl-adenosine (m⁶A). In some embodiments, the mRNA comprises N6-methyl-adenosine (m⁶A) and 5-methyl-cytidine (m⁵C).

In certain embodiments, an mRNA of the disclosure is uniformly modified (i.e., fully modified, modified through-out the entire sequence) for a particular modification. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region.

Examples of nucleoside modifications and combinations thereof that may be present in mmRNAs of the present disclosure include, but are not limited to, those described in PCT Patent Application Publications: WO2012045075, WO2014081507, WO2014093924, WO2014164253, and WO2014159813.

The mmRNAs of the disclosure can include a combination of modifications to the sugar, the nucleobase, and/or the internucleoside linkage. These combinations can include any one or more modifications described herein.

Examples of modified nucleosides and modified nucleoside combinations are provided below in Table 1 and Table 2. These combinations of modified nucleotides can be used to form the mmRNAs of the disclosure. In certain embodiments, the modified nucleosides may be partially or completely substituted for the natural nucleotides of the mRNAs of the disclosure. As a non-limiting example, the natural nucleotide uridine may be substituted with a modified nucleoside described herein. In another non-limiting example, the natural nucleoside uridine may be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9% of the natural uridines) with at least one of the modified nucleoside disclosed herein.

TABLE 1
Combinations of Nucleoside Modifications

| Modified Nucleotide | Modified Nucleotide Combination |
|---|---|
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1- |
| | methyl-pseudouridine and about 50% of uridines |
| | are pseudouridine |
| | pseudoisocytidine/about 25% of uridines are N1- |
| | methyl-pseudouridine and about 25% of uridines |
| | are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are 2- |
| | thio-uridine |
| | about 50% of uridines are 5-methyl-cytidine/about |
| | 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are N4-acetyl-cytidine/ |
| | about 50% of uridines are 2-thio-uridine |

TABLE 2
Modified Nucleosides and Combinations Thereof

1-(2,2,2-Trifluoroethyl)pseudo-UTP
1-Ethyl-pseudo-UTP
1-Methyl-pseudo-U-alpha-thio-TP
1-methyl-pseudouridine TP, ATP, GTP, CTP
1-methyl-pseudo-UTP/5-methyl-CTP/ATP/GTP
1-methyl-pseudo-UTP/CTP/ATP/GTP
1-Propyl-pseudo-UTP
25% 5-Aminoallyl-CTP + 75% CTP/25% 5-Methoxy-UTP + 75% UTP
25% 5-Aminoallyl-CTP + 75% CTP/75% 5-Methoxy-UTP + 25% UTP
25% 5-Bromo-CTP + 75% CTP/25% 5-Methoxy-UTP + 75% UTP
25% 5-Bromo-CTP + 75% CTP/75% 5-Methoxy-UTP + 25% UTP
25% 5-Bromo-CTP + 75% CTP/1-Methyl-pseudo-UTP
25% 5-Carboxy-CTP + 75% CTP/25% 5-Methoxy-UTP + 75% UTP
25% 5-Carboxy-CTP + 75% CTP/75% 5-Methoxy-UTP + 25% UTP
25% 5-Ethyl-CTP + 75% CTP/25% 5-Methoxy-UTP + 75% UTP
25% 5-Ethyl-CTP + 75% CTP/75% 5-Methoxy-UTP + 25% UTP
25% 5-Ethynyl-CTP + 75% CTP/25% 5-Methoxy-UTP + 75% UTP
25% 5-Ethynyl-CTP + 75% CTP/75% 5-Methoxy-UTP + 25% UTP
25% 5-Fluoro-CTP + 75% CTP/25% 5-Methoxy-UTP + 75% UTP
25% 5-Fluoro-CTP + 75% CTP/75% 5-Methoxy-UTP + 25% UTP
25% 5-Formyl-CTP + 75% CTP/25% 5-Methoxy-UTP + 75% UTP
25% 5-Formyl-CTP + 75% CTP/75% 5-Methoxy-UTP + 25% UTP
25% 5-Hydroxymethyl-CTP + 75% CTP/25% 5-Methoxy-UTP +
75% UTP
25% 5-Hydroxymethyl-CTP + 75% CTP/75% 5-Methoxy-UTP +
25% UTP
25% 5-Iodo-CTP + 75% CTP/25% 5-Methoxy-UTP + 75% UTP
25% 5-Iodo-CTP + 75% CTP/75% 5-Methoxy-UTP + 25% UTP
25% 5-Methoxy-CTP + 75% CTP/25% 5-Methoxy-UTP + 75% UTP
25% 5-Methoxy-CTP + 75% CTP/75% 5-Methoxy-UTP + 25% UTP
25% 5-Methyl-CTP + 75% CTP/25% 5-Methoxy-UTP + 75% 1-Methyl-
pseudo-UTP
25% 5-Methyl-CTP + 75% CTP/25% 5-Methoxy-UTP + 75% UTP
25% 5-Methyl-CTP + 75% CTP/50% 5-Methoxy-UTP + 50% 1-Methyl-
pseudo-UTP
25% 5-Methyl-CTP + 75% CTP/50% 5-Methoxy-UTP + 50% UTP
25% 5-Methyl-CTP + 75% CTP/5-Methoxy-UTP
25% 5-Methyl-CTP + 75% CTP/75% 5-Methoxy-UTP + 25% 1-Methyl-
pseudo-UTP
25% 5-Methyl-CTP + 75% CTP/75% 5-Methoxy-UTP + 25% UTP
25% 5-Phenyl-CTP + 75% CTP/25% 5-Methoxy-UTP + 75% UTP
25% 5-Phenyl-CTP + 75% CTP/75% 5-Methoxy-UTP + 25% UTP
25% 5-Trifluoromethyl-CTP + 75% CTP/25% 5-Methoxy-UTP +
75% UTP
25% 5-Trifluoromethyl-CTP + 75% CTP/75% 5-Methoxy-UTP +
25% UTP
25% 5-Trifluoromethyl-CTP + 75% CTP/1-Methyl-pseudo-UTP
25% N4-Ac-CTP + 75% CTP/25% 5-Methoxy-UTP + 75% UTP
25% N4-Ac-CTP + 75% CTP/75% 5-Methoxy-UTP + 25% UTP
25% N4-Bz-CTP + 75% CTP/25% 5-Methoxy-UTP + 75% UTP
25% N4-Bz-CTP + 75% CTP/75% 5-Methoxy-UTP + 25% UTP
25% N4-Methyl-CTP + 75% CTP/25% 5-Methoxy-UTP + 75% UTP
25% N4-Methyl-CTP + 75% CTP/75% 5-Methoxy-UTP + 25% UTP
25% Pseudo-iso-CTP + 75% CTP/25% 5-Methoxy-UTP + 75% UTP
25% Pseudo-iso-CTP + 75% CTP/75% 5-Methoxy-UTP + 25% UTP
25% 5-Bromo-CTP/75% CTP/Pseudo-UTP
25% 5-methoxy-UTP/25% 5-methyl-CTP/ATP/GTP
25% 5-methoxy-UTP/5-methyl-CTP/ATP/GTP
25% 5-methoxy-UTP/75% 5-methyl-CTP/ATP/GTP
25% 5-methoxy-UTP/CTP/ATP/GTP
25% 5-metoxy-UTP/50% 5-methyl-CTP/ATP/GTP
2-Amino-ATP
2-Thio-CTP
2-thio-pseudouridine TP, ATP, GTP, CTP
2-Thio-pseudo-UTP
2-Thio-UTP
3-Methyl-CTP
3-Methyl-pseudo-UTP
4-Thio-UTP
50% 5-Bromo-CTP + 50% CTP/1-Methyl-pseudo-UTP
50% 5-Hydroxymethyl-CTP + 50% CTP/1-Methyl-pseudo-UTP
50% 5-methoxy-UTP/5-methyl-CTP/ATP/GTP
50% 5-Methyl-CTP + 50% CTP/25% 5-Methoxy-UTP + 75% 1-Methyl-
pseudo-UTP
50% 5-Methyl-CTP + 50% CTP/25% 5-Methoxy-UTP + 75% UTP
50% 5-Methyl-CTP + 50% CTP/50% 5-Methoxy-UTP + 50% 1-Methyl-
pseudo-UTP
50% 5-Methyl-CTP + 50% CTP/50% 5-Methoxy-UTP + 50% UTP
50% 5-Methyl-CTP + 50% CTP/5-Methoxy-UTP
50% 5-Methyl-CTP + 50% CTP/75% 5-Methoxy-UTP + 25% 1-Methyl-
pseudo-UTP
50% 5-Methyl-CTP + 50% CTP/75% 5-Methoxy-UTP + 25% UTP
50% 5-Trifluoromethyl-CTP + 50% CTP/1-Methyl-pseudo-UTP
50% 5-Bromo-CTP/50% CTP/Pseudo-UTP
50% 5-methoxy-UTP/25% 5-methyl-CTP/ATP/GTP
50% 5-methoxy-UTP/50% 5-methyl-CTP/ATP/GTP
50% 5-methoxy-UTP/75% 5-methyl-CTP/ATP/GTP
50% 5-methoxy-UTP/CTP/ATP/GTP
5-Aminoallyl-CTP
5-Aminoallyl-CTP/5-Methoxy-UTP
5-Aminoallyl-UTP
5-Bromo-CTP
5-Bromo-CTP/5-Methoxy-UTP
5-Bromo-CTP/1-Methyl-pseudo-UTP
5-Bromo-CTP/Pseudo-UTP
5-bromocytidine TP, ATP, GTP, UTP
5-Bromo-UTP
5-Carboxy-CTP/5-Methoxy-UTP
5-Ethyl-CTP/5-Methoxy-UTP
5-Ethynyl-CTP/5-Methoxy-UTP
5-Fluoro-CTP/5-Methoxy-UTP
5-Formyl-CTP/5-Methoxy-UTP
5-Hydroxy-methyl-CTP/5-Methoxy-UTP
5-Hydroxymethyl-CTP
5-Hydroxymethyl-CTP/1-Methyl-pseudo-UTP
5-Hydroxymethyl-CTP/5-Methoxy-UTP
5-hydroxymethyl-cytidine TP, ATP, GTP, UTP
5-Iodo-CTP/5-Methoxy-UTP
5-Me-CTP/5-Methoxy-UTP
5-Methoxy carbonyl methyl-UTP
5-Methoxy-CTP/5-Methoxy-UTP
5-methoxy-uridine TP, ATP, GTP, UTP
5-methoxy-UTP
5-Methoxy-UTP
5-Methoxy-UTP/N6-Isopentenyl-ATP
5-methoxy-UTP/25% 5-methyl-CTP/ATP/GTP
5-methoxy-UTP/5-methyl-CTP/ATP/GTP
5-methoxy-UTP/75% 5-methyl-CTP/ATP/GTP
5-methoxy-UTP/CTP/ATP/GTP
5-Methyl-2-thio-UTP
5-Methylaminomethyl-UTP
5-Methyl-CTP/5-Methoxy-UTP
5-Methyl-CTP/5-Methoxy-UTP(cap 0)
5-Methyl-CTP/5-Methoxy-UTP(No cap)
5-Methyl-CTP/25% 5-Methoxy-UTP + 75% 1-Methyl-pseudo-UTP
5-Methyl-CTP/25% 5-Methoxy-UTP + 75% UTP
5-Methyl-CTP/50% 5-Methoxy-UTP + 50% 1-Methyl-pseudo-UTP
5-Methyl-CTP/50% 5-Methoxy-UTP + 50% UTP
5-Methyl-CTP/5-Methoxy-UTP/N6-Me-ATP
5-Methyl-CTP/75% 5-Methoxy-UTP + 25% 1-Methyl-pseudo-UTP
5-Methyl-CTP/75% 5-Methoxy-UTP + 25% UTP
5-Phenyl-CTP/5-Methoxy-UTP
5-Trifluoro-methyl-CTP/5-Methoxy-UTP
5-Trifluoromethyl-CTP
5-Trifluoromethyl-CTP/5-Methoxy-UTP
5-Trifluoromethyl-CTP/1-Methyl-pseudo-UTP
5-Trifluoromethyl-CTP/Pseudo-UTP
5-Trifluoromethyl-UTP
5-trifluromethylcytidine TP, ATP, GTP, UTP
75% 5-Aminoallyl-CTP + 25% CTP/25% 5-Methoxy-UTP + 75% UTP
75% 5-Aminoallyl-CTP + 25% CTP/75% 5-Methoxy-UTP + 25% UTP
75% 5-Bromo-CTP + 25% CTP/25% 5-Methoxy-UTP + 75% UTP
75% 5-Bromo-CTP + 25% CTP/75% 5-Methoxy-UTP + 25% UTP
75% 5-Carboxy-CTP + 25% CTP/25% 5-Methoxy-UTP + 75% UTP
75% 5-Carboxy-CTP + 25% CTP/75% 5-Methoxy-UTP + 25% UTP
75% 5-Ethyl-CTP + 25% CTP/25% 5-Methoxy-UTP + 75% UTP
75% 5-Ethyl-CTP + 25% CTP/75% 5-Methoxy-UTP + 25% UTP
75% 5-Ethynyl-CTP + 25% CTP/25% 5-Methoxy-UTP + 75% UTP
75% 5-Ethynyl-CTP + 25% CTP/75% 5-Methoxy-UTP + 25% UTP
75% 5-Fluoro-CTP + 25% CTP/25% 5-Methoxy-UTP + 75% UTP
75% 5-Fluoro-CTP + 25% CTP/75% 5-Methoxy-UTP + 25% UTP
75% 5-Formyl-CTP + 25% CTP/25% 5-Methoxy-UTP + 75% UTP
75% 5-Formyl-CTP + 25% CTP/75% 5-Methoxy-UTP + 25% UTP
75% 5-Hydroxymethyl-CTP + 25% CTP/25% 5-Methoxy-UTP +
75% UTP
75% 5-Hydroxymethyl-CTP + 25% CTP/75% 5-Methoxy-UTP +
25% UTP
75% 5-Iodo-CTP + 25% CTP/25% 5-Methoxy-UTP + 75% UTP
75% 5-Iodo-CTP + 25% CTP/75% 5-Methoxy-UTP + 25% UTP
75% 5-Methoxy-CTP + 25% CTP/25% 5-Methoxy-UTP + 75% UTP
75% 5-Methoxy-CTP + 25% CTP/75% 5-Methoxy-UTP + 25% UTP
75% 5-methoxy-UTP/5-methyl-CTP/ATP/GTP
75% 5-Methyl-CTP + 25% CTP/25% 5-Methoxy-UTP + 75% 1-Methyl-
pseudo-UTP
75% 5-Methyl-CTP + 25% CTP/25% 5-Methoxy-UTP + 75% UTP
75% 5-Methyl-CTP + 25% CTP/50% 5-Methoxy-UTP + 50% 1-Methyl-
pseudo-UTP
75% 5-Methyl-CTP + 25% CTP/50% 5-Methoxy-UTP + 50% UTP
75% 5-Methyl-CTP + 25% CTP/5-Methoxy-UTP
75% 5-Methyl-CTP + 25% CTP/75% 5-Methoxy-UTP + 25% 1-Methyl-
pseudo-UTP
75% 5-Methyl-CTP + 25% CTP/75% 5-Methoxy-UTP + 25% UTP
75% 5-Phenyl-CTP + 25% CTP/25% 5-Methoxy-UTP + 75% UTP
75% 5-Phenyl-CTP + 25% CTP/75% 5-Methoxy-UTP + 25% UTP
75% 5-Trifluoromethyl-CTP + 25% CTP/25% 5-Methoxy-UTP +
75% UTP
75% 5-Trifluoromethyl-CTP + 25% CTP/75% 5-Methoxy-UTP +
25% UTP
75% 5-Trifluoromethyl-CTP + 25% CTP/1-Methyl-pseudo-UTP
75% N4-Ac-CTP + 25% CTP/25% 5-Methoxy-UTP + 75% UTP
75% N4-Ac-CTP + 25% CTP/75% 5-Methoxy-UTP + 25% UTP
75% N4-Bz-CTP + 25% CTP/25% 5-Methoxy-UTP + 75% UTP
75% N4-Bz-CTP + 25% CTP/75% 5-Methoxy-UTP + 25% UTP
75% N4-Methyl-CTP + 25% CTP/25% 5-Methoxy-UTP + 75% UTP
75% N4-Methyl-CTP + 25% CTP/75% 5-Methoxy-UTP + 25% UTP
75% Pseudo-iso-CTP + 25% CTP/25% 5-Methoxy-UTP + 75% UTP
75% Pseudo-iso-CTP + 25% CTP/75% 5-Methoxy-UTP + 25% UTP
75% 5-Bromo-CTP/25% CTP/1-Methyl-pseudo-UTP
75% 5-Bromo-CTP/25% CTP/Pseudo-UTP
75% 5-methoxy-UTP/25% 5-methyl-CTP/ATP/GTP
75% 5-methoxy-UTP/50% 5-methyl-CTP/ATP/GTP
75% 5-methoxy-UTP/75% 5-methyl-CTP/ATP/GTP
75% 5-methoxy-UTP/CTP/ATP/GTP
8-Aza-ATP
Alpha-thio-CTP
CTP/25% 5-Methoxy-UTP + 75% 1-Methyl-pseudo-UTP
CTP/25% 5-Methoxy-UTP + 75% UTP
CTP/50% 5-Methoxy-UTP + 50% 1-Methyl-pseudo-UTP
CTP/50% 5-Methoxy-UTP + 50% UTP
CTP/5-Methoxy-UTP
CTP/5-Methoxy-UTP (cap 0)
CTP/5-Methoxy-UTP(No cap)
CTP/75% 5-Methoxy-UTP + 25% 1-Methyl-pseudo-UTP
CTP/75% 5-Methoxy-UTP + 25% UTP
CTP/UTP(No cap)
N1-Me-GTP
N4-Ac-CTP
N4Ac-CTP/1-Methyl-pseudo-UTP
N4Ac-CTP/5-Methoxy-UTP
N4-acetyl-cytidine TP, ATP, GTP, UTP
N4-Bz-CTP/5-Methoxy-UTP
N4-methyl CTP
N4-Methyl-CTP/5-Methoxy-UTP
Pseudo-iso-CTP/5-Methoxy-UTP
PseudoU-alpha-thio-TP
pseudouridine TP, ATP, GTP, CTP
pseudo-UTP/5-methyl-CTP/ATP/GTP
UTP-5-oxyacetic acid Me ester
Xanthosine

According to the disclosure, polynucleotides of the disclosure may be synthesized to comprise the combinations or single modifications of Table 1 or Table 2. Where a single modification is listed, the listed nucleoside or nucleotide represents 100 percent of that A, U, G or C nucleotide or nucleoside having been modified. Where percentages are listed, these represent the percentage of that particular A, U, G or C nucleobase triphosphate of the total amount of A, U, G, or C triphosphate present. For example, the combination: 25% 5-Aminoallyl-CTP+75% CTP/25% 5-Methoxy-UTP+75% UTP refers to a polynucleotide where 25% of the cytosine triphosphates are 5-Aminoallyl-CTP while 75% of the cytosines are CTP; whereas 25% of the uracils are 5-methoxy UTP while 75% of the uracils are UTP. Where no modified UTP is listed then the naturally occurring ATP, UTP, GTP and/or CTP is used at 100% of the sites of those nucleotides found in the polynucleotide. In this example all of the GTP and ATP nucleotides are left unmodified.

The mRNAs of the present disclosure, or regions thereof, may be codon optimized. Codon optimization methods are known in the art and may be useful for a variety of purposes: matching codon frequencies in host organisms to ensure proper folding, bias GC content to increase mRNA stability or reduce secondary structures, minimize tandem repeat codons or base runs that may impair gene construction or expression, customize transcriptional and translational control regions, insert or remove proteins trafficking sequences, remove/add post translation modification sites in encoded proteins (e.g., glycosylation sites), add, remove or shuffle protein domains, insert or delete restriction sites, modify ribosome binding sites and mRNA degradation sites, adjust translation rates to allow the various domains of the protein to fold properly, or to reduce or eliminate problem secondary structures within the polynucleotide. Codon optimization tools, algorithms and services are known in the art; non-limiting examples include services from GeneArt (Life Technologies), DNA2.0 (Menlo Park, CA) and/or proprietary methods. In one embodiment, the mRNA sequence is optimized using optimization algorithms, e.g., to optimize expression in mammalian cells or enhance mRNA stability.

In certain embodiments, the present disclosure includes polynucleotides having at least 80%, at least 85%, at least 90%, at least 95%, at least 98%, or at least 99% sequence identity to any of the polynucleotide sequences described herein.

mRNAs of the present disclosure may be produced by means available in the art, including but not limited to in vitro transcription (IVT) and synthetic methods. Enzymatic (IVT), solid-phase, liquid-phase, combined synthetic methods, small region synthesis, and ligation methods may be utilized. In one embodiment, mRNAs are made using IVT enzymatic synthesis methods. Methods of making polynucleotides by IVT are known in the art and are described in International Application PCT/US2013/30062, the contents of which are incorporated herein by reference in their entirety. Accordingly, the present disclosure also includes polynucleotides, e.g., DNA, constructs (e.g., plamids) and vectors (e.g., viral vectors) that may be used to in vitro transcribe an mRNA described herein.

Non-natural modified nucleobases may be introduced into polynucleotides, e.g., mRNA, during synthesis or post-synthesis. In certain embodiments, modifications may be on internucleoside linkages, purine or pyrimidine bases, or sugar. In particular embodiments, the modification may be introduced at the terminal of a polynucleotide chain or anywhere else in the polynucleotide chain; with chemical synthesis or with a polymerase enzyme. Examples of modified nucleic acids and their synthesis are disclosed in PCT application No. PCT/US2012/058519. Synthesis of modified polynucleotides is also described in Verma and Eckstein, Annual Review of Biochemistry, vol. 76, 99-134 (1998).

Either enzymatic or chemical ligation methods may be used to conjugate polynucleotides or their regions with different functional moieties, such as targeting or delivery agents, fluorescent labels, liquids, nanoparticles, etc. Conjugates of polynucleotides and modified polynucleotides are reviewed in Goodchild, Bioconjugate Chemistry, vol. 1 (3), 165-187 (1990).

MicroRNA (miRNA) Binding Sites

Polynucleotides of the disclosure can include regulatory elements, for example, microRNA (miRNA) binding sites, transcription factor binding sites, structured mRNA sequences and/or motifs, artificial binding sites engineered to act as pseudo-receptors for endogenous nucleic acid binding molecules, and combinations thereof. In some embodiments, polynucleotides including such regulatory elements are referred to as including “sensor sequences.” Non-limiting examples of sensor sequences are described in U.S. Publication 2014/0200261, the contents of which are incorporated herein by reference in their entirety.

In some embodiments, a polynucleotide (e.g., a ribonucleic acid (RNA), e.g., a messenger RNA (mRNA)) of the disclosure comprises an open reading frame (ORF) encoding a polypeptide of interest and further comprises one or more miRNA binding site(s). Inclusion or incorporation of miRNA binding site(s) provides for regulation of polynucleotides of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27 (1): 91-105. miRNA profiling of the target cells or tissues can be conducted to determine the presence or absence of miRNA in the cells or tissues. In some embodiments, a polynucleotide (e.g., a ribonucleic acid (RNA), e.g., a messenger RNA (mRNA)) of the disclosure comprises one or more microRNA binding sites, microRNA target sequences, microRNA complementary sequences, or microRNA seed complementary sequences. Such sequences can correspond to, e.g., have complementarity to, any known microRNA such as those taught in US Publication US2005/0261218 and US Publication US2005/0059005, the contents of each of which are incorporated herein by reference in their entirety.

As used herein, the term “microRNA (miRNA or miR) binding site” refers to a sequence within a polynucleotide, e.g., within a DNA or within an RNA transcript, including in the 5′UTR and/or 3′UTR, that has sufficient complementarity to all or a region of a miRNA to interact with, associate with or bind to the miRNA. In some embodiments, a polynucleotide of the disclosure comprising an ORF encoding a polypeptide of interest and further comprises one or more miRNA binding site(s). In exemplary embodiments, a 5′UTR and/or 3′UTR of the polynucleotide (e.g., a ribonucleic acid (RNA), e.g., a messenger RNA (mRNA)) comprises the one or more miRNA binding site(s).

A miRNA binding site having sufficient complementarity to a miRNA refers to a degree of complementarity sufficient to facilitate miRNA-mediated regulation of a polynucleotide, e.g., miRNA-mediated translational repression or degradation of the polynucleotide. In exemplary aspects of the disclosure, a miRNA binding site having sufficient complementarity to the miRNA refers to a degree of complementarity sufficient to facilitate miRNA-mediated degradation of the polynucleotide, e.g., miRNA-guided RNA-induced silencing complex (RISC)-mediated cleavage of mRNA. The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence. Full or complete complementarity (e.g., full complementarity or complete complementarity over all or a significant portion of the length of a naturally-occurring miRNA) is preferred when the desired regulation is mRNA degradation.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation.

In some embodiments, the miRNA binding site binds the corresponding mature miRNA that is part of an active RISC containing Dicer. In another embodiment, binding of the miRNA binding site to the corresponding miRNA in RISC degrades the mRNA containing the miRNA binding site or prevents the mRNA from being translated. In some embodiments, the miRNA binding site has sufficient complementarity to miRNA so that a RISC complex comprising the miRNA cleaves the polynucleotide comprising the miRNA binding site. In other embodiments, the miRNA binding site has imperfect complementarity so that a RISC complex comprising the miRNA induces instability in the polynucleotide comprising the miRNA binding site. In another embodiment, the miRNA binding site has imperfect complementarity so that a RISC complex comprising the miRNA represses transcription of the polynucleotide comprising the miRNA binding site.

In some embodiments, the miRNA binding site has one, two, three, four, five, six, seven, eight, nine, ten, eleven or twelve mismatch(es) from the corresponding miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

By engineering one or more miRNA binding sites into a polynucleotide of the disclosure, the polynucleotide can be targeted for degradation or reduced translation, provided the miRNA in question is available. This can reduce off-target effects upon delivery of the polynucleotide. For example, if a polynucleotide of the disclosure is not intended to be delivered to a tissue or cell but ends up is said tissue or cell, then a miRNA abundant in the tissue or cell can inhibit the expression of the gene of interest if one or multiple binding sites of the miRNA are engineered into the 5′UTR and/or 3′UTR of the polynucleotide.

Conversely, miRNA binding sites can be removed from polynucleotide sequences in which they naturally occur in order to increase protein expression in specific tissues. For example, a binding site for a specific miRNA can be removed from a polynucleotide to improve protein expression in tissues or cells containing the miRNA.

In one embodiment, a polynucleotide of the disclosure can include at least one miRNA-binding site in the 5′UTR and/or 3′UTR in order to regulate cytotoxic or cytoprotective mRNA therapeutics to specific cells such as, but not limited to, normal and/or cancerous cells. In another embodiment, a polynucleotide of the disclosure can include two, three, four, five, six, seven, eight, nine, ten, or more miRNA-binding sites in the 5′-UTR and/or 3′-UTR in order to regulate cytotoxic or cytoprotective mRNA therapeutics to specific cells such as, but not limited to, normal and/or cancerous cells.

Regulation of expression in multiple tissues can be accomplished through introduction or removal of one or more miRNA binding sites, e.g., one or more distinct miRNA binding sites. The decision whether to remove or insert a miRNA binding site can be made based on miRNA expression patterns and/or their profilings in tissues and/or cells in development and/or disease. Identification of miRNAs, miRNA binding sites, and their expression patterns and role in biology have been reported (e.g., Bonauer et al., Curr Drug Targets 2010 11:943-949; Anand and Cheresh Curr Opin Hematol 2011 18:171-176; Contreras and Rao Leukemia 2012 26:404-413 (2011 Dec. 20. doi: 10.1038/leu.2011.356); Bartel Cell 2009 136:215-233; Landgraf et al, Cell, 2007 129:1401-1414; Gentner and Naldini, Tissue Antigens. 2012 80:393-403 and all references therein; each of which is incorporated herein by reference in its entirety). miRNAs and miRNA binding sites can correspond to any known sequence, including non-limiting examples described in U.S. Publication Nos. 2014/0200261, 2005/0261218, and 2005/0059005, each of which are incorporated herein by reference in their entirety.

Examples of tissues where miRNA are known to regulate mRNA, and thereby protein expression, include, but are not limited to, liver (miR-122), muscle (miR-133, miR-206, miR-208), endothelial cells (miR-17-92, miR-126), myeloid cells (miR-142-3p, miR-142-5p, miR-16, miR-21, miR-223, miR-24, miR-27), adipose tissue (let-7, miR-30c), heart (miR-1d, miR-149), kidney (miR-192, miR-194, miR-204), and lung epithelial cells (let-7, miR-133, miR-126).

Specifically, miRNAs are known to be differentially expressed in immune cells (also called hematopoietic cells), such as antigen presenting cells (APCs) (e.g., dendritic cells and macrophages), macrophages, monocytes, B lymphocytes, T lymphocytes, granulocytes, natural killer cells, etc. Immune cell specific miRNAs are involved in immunogenicity, autoimmunity, the immune response to infection, inflammation, as well as unwanted immune response after gene therapy and tissue/organ transplantation. Immune cell specific miRNAs also regulate many aspects of development, proliferation, differentiation and apoptosis of hematopoietic cells (immune cells). For example, miR-142 and miR-146 are exclusively expressed in immune cells, particularly abundant in myeloid dendritic cells. It has been demonstrated that the immune response to a polynucleotide can be shut-off by adding miR-142 binding sites to the 3′-UTR of the polynucleotide, enabling more stable gene transfer in tissues and cells. miR-142 efficiently degrades exogenous polynucleotides in antigen presenting cells and suppresses cytotoxic elimination of transduced cells (e.g., Annoni A et al., blood, 2009, 114, 5152-5161; Brown B D, et al., Nat med. 2006, 12 (5), 585-591; Brown B D, et al., blood, 2007, 110(13):4144-4152, each of which is incorporated herein by reference in its entirety).

An antigen-mediated immune response can refer to an immune response triggered by foreign antigens, which, when entering an organism, are processed by the antigen presenting cells and displayed on the surface of the antigen presenting cells. T cells can recognize the presented antigen and induce a cytotoxic elimination of cells that express the antigen.

Introducing a miR-142 binding site into the 5′UTR and/or 3′UTR of a polynucleotide of the disclosure can selectively repress gene expression in antigen presenting cells through miR-142 mediated degradation, limiting antigen presentation in antigen presenting cells (e.g., dendritic cells) and thereby preventing antigen-mediated immune response after the delivery of the polynucleotide. The polynucleotide is then stably expressed in target tissues or cells without triggering cytotoxic elimination.

In one embodiment, binding sites for miRNAs that are known to be expressed in immune cells, in particular, antigen presenting cells, can be engineered into a polynucleotide of the disclosure to suppress the expression of the polynucleotide in antigen presenting cells through miRNA mediated RNA degradation, subduing the antigen-mediated immune response. Expression of the polynucleotide is maintained in non-immune cells where the immune cell specific miRNAs are not expressed. For example, in some embodiments, to prevent an immunogenic reaction against a liver specific protein, any miR-122 binding site can be removed and a miR-142 (and/or mirR-146) binding site can be engineered into the 5′UTR and/or 3′UTR of a polynucleotide of the disclosure.

To further drive the selective degradation and suppression in APCs and macrophage, a polynucleotide of the disclosure can include a further negative regulatory element in the 5′UTR and/or 3′UTR, either alone or in combination with miR-142 and/or miR-146 binding sites. As a non-limiting example, the further negative regulatory element is a Constitutive Decay Element (CDE).

Immune cell specific miRNAs include, but are not limited to, hsa-let-7a-2-3p, hsa-let-7a-3p, hsa-7a-5p, hsa-let-7c, hsa-let-7e-3p, hsa-let-7e-5p, hsa-let-7g-3p, hsa-let-7g-5p, hsa-let-7i-3p, hsa-let-7i-5p, miR-10a-3p, miR-10a-5p, miR-1184, hsa-let-7f-1-3p, hsa-let-7f-2-5p, hsa-let-7f-5p, miR-125b-1-3p, miR-125b-2-3p, miR-125b-5p, miR-1279, miR-130a-3p, miR-130a-5p, miR-132-3p, miR-132-5p, miR-142-3p, miR-142-5p, miR-143-3p, miR-143-5p, miR-146a-3p, miR-146a-5p, miR-146b-3p, miR-146b-5p, miR-147a, miR-147b, miR-148a-5p, miR-148a-3p, miR-150-3p, miR-150-5p, miR-151b, miR-155-3p, miR-155-5p, miR-15a-3p, miR-15a-5p, miR-15b-5p, miR-15b-3p, miR-16-1-3p, miR-16-2-3p, miR-16-5p, miR-17-5p, miR-181a-3p, miR-181a-5p, miR-181a-2-3p, miR-182-3p, miR-182-5p, miR-197-3p, miR-197-5p, miR-21-5p, miR-21-3p, miR-214-3p, miR-214-5p, miR-223-3p, miR-223-5p, miR-221-3p, miR-221-5p, miR-23b-3p, miR-23b-5p, miR-24-1-5p, miR-24-2-5p, miR-24-3p, miR-26a-1-3p, miR-26a-2-3p, miR-26a-5p, miR-26b-3p, miR-26b-5p, miR-27a-3p, miR-27a-5p, miR-27b-3p, miR-27b-5p, miR-28-3p, miR-28-5p, miR-2909, miR-29a-3p, miR-29a-5p, miR-29b-1-5p, miR-29b-2-5p, miR-29c-3p, miR-29c-5p, miR-30e-3p, miR-30e-5p, miR-331-5p, miR-339-3p, miR-339-5p, miR-345-3p, miR-345-5p, miR-346, miR-34a-3p, miR-34a-5p, miR-363-3p, miR-363-5p, miR-372, miR-377-3p, miR-377-5p, miR-493-3p, miR-493-5p, miR-542, miR-548b-5p, miR548c-5p, miR-548i, miR-548j, miR-548n, miR-574-3p, miR-598, miR-718, miR-935, miR-99a-3p, miR-99a-5p, miR-99b-3p, and miR-99b-5p. Furthermore, novel miRNAs can be identified in immune cell through micro-array hybridization and microtome analysis (e.g., Jima D D et al, Blood, 2010, 116:e118-e127; Vaz C et al., BMC Genomics, 2010, 11,288, the content of each of which is incorporated herein by reference in its entirety.)

miRNAs that are known to be expressed in the liver include, but are not limited to, miR-107, miR-122-3p, miR-122-5p, miR-1228-3p, miR-1228-5p, miR-1249, miR-129-5p, miR-1303, miR-151a-3p, miR-151a-5p, miR-152, miR-194-3p, miR-194-5p, miR-199a-3p, miR-199a-5p, miR-199b-3p, miR-199b-5p, miR-296-5p, miR-557, miR-581, miR-939-3p, and miR-939-5p. miRNA binding sites from any liver specific miRNA can be introduced to or removed from a polynucleotide of the disclosure to regulate expression of the polynucleotide in the liver. Liver specific miRNA binding sites can be engineered alone or further in combination with immune cell (e.g., APC) miRNA binding sites in a polynucleotide of the disclosure.

miRNAs that are known to be expressed in the lung include, but are not limited to, let-7a-2-3p, let-7a-3p, let-7a-5p, miR-126-3p, miR-126-5p, miR-127-3p, miR-127-5p, miR-130a-3p, miR-130a-5p, miR-130b-3p, miR-130b-5p, miR-133a, miR-133b, miR-134, miR-18a-3p, miR-18a-5p, miR-18b-3p, miR-18b-5p, miR-24-1-5p, miR-24-2-5p, miR-24-3p, miR-296-3p, miR-296-5p, miR-32-3p, miR-337-3p, miR-337-5p, miR-381-3p, and miR-381-5p. miRNA binding sites from any lung specific miRNA can be introduced to or removed from a polynucleotide of the disclosure to regulate expression of the polynucleotide in the lung. Lung specific miRNA binding sites can be engineered alone or further in combination with immune cell (e.g., APC) miRNA binding sites in a polynucleotide of the disclosure.

miRNAs that are known to be expressed in the heart include, but are not limited to, miR-1, miR-133a, miR-133b, miR-149-3p, miR-149-5p, miR-186-3p, miR-186-5p, miR-208a, miR-208b, miR-210, miR-296-3p, miR-320, miR-451a, miR-451b, miR-499a-3p, miR-499a-5p, miR-499b-3p, miR-499b-5p, miR-744-3p, miR-744-5p, miR-92b-3p, and miR-92b-5p. miRNA binding sites from any heart specific microRNA can be introduced to or removed from a polynucleotide of the disclosure to regulate expression of the polynucleotide in the heart. Heart specific miRNA binding sites can be engineered alone or further in combination with immune cell (e.g., APC) miRNA binding sites in a polynucleotide of the disclosure.

miRNAs that are known to be expressed in the nervous system include, but are not limited to, miR-124-5p, miR-125a-3p, miR-125a-5p, miR-125b-1-3p, miR-125b-2-3p, miR-125b-5p, miR-1271-3p, miR-1271-5p, miR-128, miR-132-5p, miR-135a-3p, miR-135a-5p, miR-135b-3p, miR-135b-5p, miR-137, miR-139-5p, miR-139-3p, miR-149-3p, miR-149-5p, miR-153, miR-181c-3p, miR-181c-5p, miR-183-3p, miR-183-5p, miR-190a, miR-190b, miR-212-3p, miR-212-5p, miR-219-1-3p, miR-219-2-3p, miR-23a-3p, miR-23a-5p, miR-30a-5p, miR-30b-3p, miR-30b-5p, miR-30c-1-3p, miR-30c-2-3p, miR-30c-5p, miR-30d-3p, miR-30d-5p, miR-329, miR-342-3p, miR-3665, miR-3666, miR-380-3p, miR-380-5p, miR-383, miR-410, miR-425-3p, miR-425-5p, miR-454-3p, miR-454-5p, miR-483, miR-510, miR-516a-3p, miR-548b-5p, miR-548c-5p, miR-571, miR-7-1-3p, miR-7-2-3p, miR-7-5p, miR-802, miR-922, miR-9-3p, and miR-9-5p. miRNAs enriched in the nervous system further include those specifically expressed in neurons, including, but not limited to, miR-132-3p, miR-132-3p, miR-148b-3p, miR-148b-5p, miR-151a-3p, miR-151a-5p, miR-212-3p, miR-212-5p, miR-320b, miR-320e, miR-323a-3p, miR-323a-5p, miR-324-5p, miR-325, miR-326, miR-328, miR-922 and those specifically expressed in glial cells, including, but not limited to, miR-1250, miR-219-1-3p, miR-219-2-3p, miR-219-5p, miR-23a-3p, miR-23a-5p, miR-3065-3p, miR-3065-5p, miR-30e-3p, miR-30e-5p, miR-32-5p, miR-338-5p, and miR-657. miRNA binding sites from any CNS specific miRNA can be introduced to or removed from a polynucleotide of the disclosure to regulate expression of the polynucleotide in the nervous system. Nervous system specific miRNA binding sites can be engineered alone or further in combination with immune cell (e.g., APC) miRNA binding sites in a polynucleotide of the disclosure.

miRNAs that are known to be expressed in the pancreas include, but are not limited to, miR-105-3p, miR-105-5p, miR-184, miR-195-3p, miR-195-5p, miR-196a-3p, miR-196a-5p, miR-214-3p, miR-214-5p, miR-216a-3p, miR-216a-5p, miR-30a-3p, miR-33a-3p, miR-33a-5p, miR-375, miR-7-1-3p, miR-7-2-3p, miR-493-3p, miR-493-5p, and miR-944. miRNA binding sites from any pancreas specific miRNA can be introduced to or removed from a polynucleotide of the disclosure to regulate expression of the polynucleotide in the pancreas. Pancreas specific miRNA binding sites can be engineered alone or further in combination with immune cell (e.g. APC) miRNA binding sites in a polynucleotide of the disclosure.

miRNAs that are known to be expressed in the kidney include, but are not limited to, miR-122-3p, miR-145-5p, miR-17-5p, miR-192-3p, miR-192-5p, miR-194-3p, miR-194-5p, miR-20a-3p, miR-20a-5p, miR-204-3p, miR-204-5p, miR-210, miR-216a-3p, miR-216a-5p, miR-296-3p, miR-30a-3p, miR-30a-5p, miR-30b-3p, miR-30b-5p, miR-30c-1-3p, miR-30c-2-3p, miR30c-5p, miR-324-3p, miR-335-3p, miR-335-5p, miR-363-3p, miR-363-5p, and miR-562. miRNA binding sites from any kidney specific miRNA can be introduced to or removed from a polynucleotide of the disclosure to regulate expression of the polynucleotide in the kidney. Kidney specific miRNA binding sites can be engineered alone or further in combination with immune cell (e.g., APC) miRNA binding sites in a polynucleotide of the disclosure.

miRNAs that are known to be expressed in the muscle include, but are not limited to, let-7g-3p, let-7g-5p, miR-1, miR-1286, miR-133a, miR-133b, miR-140-3p, miR-143-3p, miR-143-5p, miR-145-3p, miR-145-5p, miR-188-3p, miR-188-5p, miR-206, miR-208a, miR-208b, miR-25-3p, and miR-25-5p. miRNA binding sites from any muscle specific miRNA can be introduced to or removed from a polynucleotide of the disclosure to regulate expression of the polynucleotide in the muscle. Muscle specific miRNA binding sites can be engineered alone or further in combination with immune cell (e.g., APC) miRNA binding sites in a polynucleotide of the disclosure.

miRNAs are also differentially expressed in different types of cells, such as, but not limited to, endothelial cells, epithelial cells, and adipocytes.

miRNAs that are known to be expressed in endothelial cells include, but are not limited to, let-7b-3p, let-7b-5p, miR-100-3p, miR-100-5p, miR-101-3p, miR-101-5p, miR-126-3p, miR-126-5p, miR-1236-3p, miR-1236-5p, miR-130a-3p, miR-130a-5p, miR-17-5p, miR-17-3p, miR-18a-3p, miR-18a-5p, miR-19a-3p, miR-19a-5p, miR-19b-1-5p, miR-19b-2-5p, miR-19b-3p, miR-20a-3p, miR-20a-5p, miR-217, miR-210, miR-21-3p, miR-21-5p, miR-221-3p, miR-221-5p, miR-222-3p, miR-222-5p, miR-23a-3p, miR-23a-5p, miR-296-5p, miR-361-3p, miR-361-5p, miR-421, miR-424-3p, miR-424-5p, miR-513a-5p, miR-92a-1-5p, miR-92a-2-5p, miR-92a-3p, miR-92b-3p, and miR-92b-5p. Many novel miRNAs are discovered in endothelial cells from deep-sequencing analysis (e.g., Voellenkle C et al., RNA, 2012, 18, 472-484, herein incorporated by reference in its entirety). miRNA binding sites from any endothelial cell specific miRNA can be introduced to or removed from a polynucleotide of the disclosure to regulate expression of the polynucleotide in the endothelial cells.

miRNAs that are known to be expressed in epithelial cells include, but are not limited to, let-7b-3p, let-7b-5p, miR-1246, miR-200a-3p, miR-200a-5p, miR-200b-3p, miR-200b-5p, miR-200c-3p, miR-200c-5p, miR-338-3p, miR-429, miR-451a, miR-451b, miR-494, miR-802 and miR-34a, miR-34b-5p, miR-34c-5p, miR-449a, miR-449b-3p, miR-449b-5p specific in respiratory ciliated epithelial cells, let-7 family, miR-133a, miR-133b, miR-126 specific in lung epithelial cells, miR-382-3p, miR-382-5p specific in renal epithelial cells, and miR-762 specific in corneal epithelial cells. miRNA binding sites from any epithelial cell specific miRNA can be introduced to or removed from a polynucleotide of the disclosure to regulate expression of the polynucleotide in the epithelial cells.

In addition, a large group of miRNAs are enriched in embryonic stem cells, controlling stem cell self-renewal as well as the development and/or differentiation of various cell lineages, such as neural cells, cardiac, hematopoietic cells, skin cells, osteogenic cells and muscle cells (e.g., Kuppusamy K T et al., Curr. Mol Med, 2013, 13 (5), 757-764; Vidigal J A and Ventura A, Semin Cancer Biol. 2012, 22 (5-6), 428-436; Goff L A et al., PLOS One, 2009, 4:e7192; Morin R D et al., Genome Res, 2008,18, 610-621; Yoo J K et al., Stem Cells Dev. 2012, 21 (11), 2049-2057, each of which is herein incorporated by reference in its entirety). miRNAs abundant in embryonic stem cells include, but are not limited to, let-7a-2-3p, let-a-3p, let-7a-5p, let7d-3p, let-7d-5p, miR-103a-2-3p, miR-103a-5p, miR-106b-3p, miR-106b-5p, miR-1246, miR-1275, miR-138-1-3p, miR-138-2-3p, miR-138-5p, miR-154-3p, miR-154-5p, miR-200c-3p, miR-200c-5p, miR-290, miR-301a-3p, miR-301a-5p, miR-302a-3p, miR-302a-5p, miR-302b-3p, miR-302b-5p, miR-302c-3p, miR-302c-5p, miR-302d-3p, miR-302d-5p, miR-302e, miR-367-3p, miR-367-5p, miR-369-3p, miR-369-5p, miR-370, miR-371, miR-373, miR-380-5p, miR-423-3p, miR-423-5p, miR-486-5p, miR-520c-3p, miR-548e, miR-548f, miR-548g-3p, miR-548g-5p, miR-548i, miR-548k, miR-5481, miR-548m, miR-548n, miR-5480-3p, miR-5480-5p, miR-548p, miR-664a-3p, miR-664a-5p, miR-664b-3p, miR-664b-5p, miR-766-3p, miR-766-5p, miR-885-3p, miR-885-5p, miR-93-3p, miR-93-5p, miR-941,miR-96-3p, miR-96-5p, miR-99b-3p and miR-99b-5p. Many predicted novel miRNAs are discovered by deep sequencing in human embryonic stem cells (e.g., Morin R D et al., Genome Res, 2008,18, 610-621; Goff L A et al., PLOS One, 2009, 4:e7192; Bar M et al., Stem cells, 2008, 26, 2496-2505, the content of each of which is incorporated herein by reference in its entirety).

In one embodiment, the binding sites of embryonic stem cell specific miRNAs can be included in or removed from the 3′UTR of a polynucleotide of the disclosure to modulate the development and/or differentiation of embryonic stem cells, to inhibit the senescence of stem cells in a degenerative condition (e.g. degenerative diseases), or to stimulate the senescence and apoptosis of stem cells in a disease condition (e.g. cancer stem cells).

Many miRNA expression studies are conducted to profile the differential expression of miRNAs in various cancer cells/tissues and other diseases. Some miRNAs are abnormally over-expressed in certain cancer cells and others are under-expressed. For example, miRNAs are differentially expressed in cancer cells (WO2008/154098, US2013/0059015, US2013/0042333, WO2011/157294); cancer stem cells (US2012/0053224); pancreatic cancers and diseases (US2009/0131348, US2011/0171646, US2010/0286232, U.S. Pat. No. 8,389,210); asthma and inflammation (U.S. Pat. No. 8,415,096); prostate cancer (US2013/0053264); hepatocellular carcinoma (WO2012/151212, US2012/0329672, WO2008/054828, U.S. Pat. No. 8,252,538); lung cancer cells (WO2011/076143, WO2013/033640, WO2009/070653, US2010/0323357); cutaneous T cell lymphoma (WO2013/011378); colorectal cancer cells (WO2011/0281756, WO2011/076142); cancer positive lymph nodes (WO2009/100430, US2009/0263803); nasopharyngeal carcinoma (EP2112235); chronic obstructive pulmonary disease (US2012/0264626, US2013/0053263); thyroid cancer (WO2013/066678); ovarian cancer cells (US2012/0309645, WO2011/095623); breast cancer cells (WO2008/154098, WO2007/081740, US2012/0214699), leukemia and lymphoma (WO2008/073915, US2009/0092974, US2012/0316081, US2012/0283310, WO2010/018563), the content of each of which is incorporated herein by reference in its entirety.

As a non-limiting example, miRNA binding sites for miRNAs that are over-expressed in certain cancer and/or tumor cells can be removed from the 3′UTR of a polynucleotide of the disclosure, restoring the expression suppressed by the over-expressed miRNAs in cancer cells, thus ameliorating the corresponsive biological function, for instance, transcription stimulation and/or repression, cell cycle arrest, apoptosis and cell death. Normal cells and tissues, wherein miRNAs expression is not up-regulated, will remain unaffected.

miRNA can also regulate complex biological processes such as angiogenesis (e.g., miR-132) (Anand and Cheresh Curr Opin Hematol 2011 18:171-176). In the polynucleotides of the disclosure, miRNA binding sites that are involved in such processes can be removed or introduced, in order to tailor the expression of the polynucleotides to biologically relevant cell types or relevant biological processes. In this context, the polynucleotides of the disclosure are defined as auxotrophic polynucleotides.

In some embodiments, the therapeutic window and/or differential expression (e.g., tissue-specific expression) of a polypeptide of the disclosure may be altered by incorporation of a miRNA binding site into an mRNA encoding the polypeptide. In one example, an mRNA may include one or more miRNA binding sites that are bound by miRNAs that have higher expression in one tissue type as compared to another. In another example, an mRNA may include one or more miRNA binding sites that are bound by miRNAs that have lower expression in a cancer cell as compared to a non-cancerous cell of the same tissue of origin. When present in a cancer cell that expresses low levels of such an miRNA, the polypeptide encoded by the mRNA typically will show increased expression.

Liver cancer cells (e.g., hepatocellular carcinoma cells) typically express low levels of miR-122 as compared to normal liver cells. Therefore, an mRNA encoding a polypeptide that includes at least one miR-122 binding site (e.g., in the 3′-UTR of the mRNA) will typically express comparatively low levels of the polypeptide in normal liver cells and comparatively high levels of the polypeptide in liver cancer cells. If the polypeptide is able to induce immunogenic cell death, this can cause preferential immunogenic cell killing of liver cancer cells (e.g., hepatocellular carcinoma cells) as compared to normal liver cells.

In some embodiments, the mRNA includes at least one miR-122 binding site, at least two miR-122 binding sites, at least three miR-122 binding sites, at least four miR-122 binding sites, or at least five miR-122 binding sites. In one aspect, the miRNA binding site binds miR-122 or is complementary to miR-122. In another aspect, the miRNA binding site binds to miR-122-3p or miR-122-5p. In a particular aspect, the miRNA binding site comprises a nucleotide sequence at least 80%, at least 85%, at least 90%, at least 95%, or 100% identical to SEQ ID NO: 1326, wherein the miRNA binding site binds to miR-122. In another particular aspect, the miRNA binding site comprises a nucleotide sequence at least 80%, at least 85%, at least 90%, at least 95%, or 100% identical to SEQ ID NO: 26, wherein the miRNA binding site binds to miR-122. These sequences are shown below in Table 3.

In some embodiments, a polynucleotide of the disclosure comprises a miRNA binding site, wherein the miRNA binding site comprises one or more nucleotide sequences selected from Table 3, including one or more copies of any one or more of the miRNA binding site sequences. In some embodiments, a polynucleotide of the disclosure further comprises at least one, two, three, four, five, six, seven, eight, nine, ten, or more of the same or different miRNA binding sites selected from Table 3, including any combination thereof. In some embodiments, the miRNA binding site binds to miR-142 or is complementary to miR-142. In some embodiments, the miR-142 comprises SEQ ID NO: 27. In some embodiments, the miRNA binding site binds to miR-142-3p or miR-142-5p. In some embodiments, the miR-142-3p binding site comprises SEQ ID NO: 29. In some embodiments, the miR-142-5p binding site comprises SEQ ID NO: 31. In some embodiments, the miRNA binding site comprises a nucleotide sequence at least 80%, at least 85%, at least 90%, at least 95%, or 100% identical to SEQ ID NO: 29 or SEQ ID NO: 31.

TABLE 3
Representative microRNAs and microRNA
binding sites

| SEQ | | |
|---|---|---|
| ID | | |
| NO. | Description | Sequence |
| 27 | miR-142 | GACAGUGCAGUCACCCAUAAAGUAGAAAG |
| | | CACUACUAACAGCACUGGAGGGUGUAGUG |
| | | UUUCCUACUUUAUGGAUGAGUGUACUGUG |
| 28 | miR-142-3p | UGUAGUGUUUCCUACUUUAUGGA |
| 29 | miR-142-3p | uCCAUAAAGUAGGAAACACUACA |
| | binding site | |
| 30 | miR-142-5p | CAUAAAGUAGAAAGCACUACU |
| 31 | miR-142-5p | AGUAGUGCUUUCUACUUUAUG |
| | binding site | |
| 1324 | miR-122 | CCUUAGCAGAGCUGUGGAGUGUGACAAUG |
| | | GUGUUUGUGUCUAAACUAUCAAACGCCAU |
| | | UAUCACACUAAAUAGCUACUGCUAGGC |
| 32 | miR-122-3p | AACGCCAUUAUCACACUAAAUA |
| 1325 | miR-122-3p | UAUUUAGUGUGAUAAUGGCGUU |
| | binding site | |
| 33 | miR-122-5p | UGGAGUGUGACAAUGGUGUUUG |
| 1326 | miR-122-5p | CAAACACCAUUGUCACACUCCA |
| | binding site | |

In some embodiments, a miRNA binding site is inserted in the polynucleotide of the disclosure in any position of the polynucleotide (e.g., the 5′UTR and/or 3′UTR). In some embodiments, the 5′UTR comprises a miRNA binding site. In some embodiments, the 3′UTR comprises a miRNA binding site. In some embodiments, the 5′UTR and the 3′UTR comprise a miRNA binding site. The insertion site in the polynucleotide can be anywhere in the polynucleotide as long as the insertion of the miRNA binding site in the polynucleotide does not interfere with the translation of a functional polypeptide in the absence of the corresponding miRNA; and in the presence of the miRNA, the insertion of the miRNA binding site in the polynucleotide and the binding of the miRNA binding site to the corresponding miRNA are capable of degrading the polynucleotide or preventing the translation of the polynucleotide.

In some embodiments, a miRNA binding site is inserted in at least about 30 nucleotides downstream from the stop codon of an ORF in a polynucleotide of the disclosure comprising the ORF. In some embodiments, a miRNA binding site is inserted in at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, at least about 50 nucleotides, at least about 55 nucleotides, at least about 60 nucleotides, at least about 65 nucleotides, at least about 70 nucleotides, at least about 75 nucleotides, at least about 80 nucleotides, at least about 85 nucleotides, at least about 90 nucleotides, at least about 95 nucleotides, or at least about 100 nucleotides downstream from the stop codon of an ORF in a polynucleotide of the disclosure. In some embodiments, a miRNA binding site is inserted in about 10 nucleotides to about 100 nucleotides, about 20 nucleotides to about 90 nucleotides, about 30 nucleotides to about 80 nucleotides, about 40 nucleotides to about 70 nucleotides, about 50 nucleotides to about 60 nucleotides, about 45 nucleotides to about 65 nucleotides downstream from the stop codon of an ORF in a polynucleotide of the disclosure.

miRNA gene regulation can be influenced by the sequence surrounding the miRNA such as, but not limited to, the species of the surrounding sequence, the type of sequence (e.g., heterologous, homologous, exogenous, endogenous, or artificial), regulatory elements in the surrounding sequence and/or structural elements in the surrounding sequence. The miRNA can be influenced by the 5′UTR and/or 3′UTR. As a non-limiting example, a non-human 3′UTR can increase the regulatory effect of the miRNA sequence on the expression of a polypeptide of interest compared to a human 3′UTR of the same sequence type.

In one embodiment, other regulatory elements and/or structural elements of the 5′UTR can influence miRNA mediated gene regulation. One example of a regulatory element and/or structural element is a structured IRES (Internal Ribosome Entry Site) in the 5′UTR, which is necessary for the binding of translational elongation factors to initiate protein translation. EIF4A2 binding to this secondarily structured element in the 5′-UTR is necessary for miRNA mediated gene expression (Meijer H A et al., Science, 2013, 340, 82-85, herein incorporated by reference in its entirety). The polynucleotides of the disclosure can further include this structured 5′UTR in order to enhance microRNA mediated gene regulation.

At least one miRNA binding site can be engineered into the 3′UTR of a polynucleotide of the disclosure. In this context, at least two, at least three, at least four, at least five, at least six, at least seven, at least eight, at least nine, at least ten, or more miRNA binding sites can be engineered into a 3′UTR of a polynucleotide of the disclosure. For example, 1 to 10, 1 to 9, 1 to 8, 1 to 7, 1 to 6, 1 to 5, 1 to 4, 1 to 3, 2, or 1 miRNA binding sites can be engineered into the 3′UTR of a polynucleotide of the disclosure. In one embodiment, miRNA binding sites incorporated into a polynucleotide of the disclosure can be the same or can be different miRNA sites. A combination of different miRNA binding sites incorporated into a polynucleotide of the disclosure can include combinations in which more than one copy of any of the different miRNA sites are incorporated. In another embodiment, miRNA binding sites incorporated into a polynucleotide of the disclosure can target the same or different tissues in the body. As a non-limiting example, through the introduction of tissue-, cell-type-, or disease-specific miRNA binding sites in the 3′-UTR of a polynucleotide of the disclosure, the degree of expression in specific cell types (e.g., hepatocytes, myeloid cells, endothelial cells, cancer cells, etc.) can be reduced.

In one embodiment, a miRNA binding site can be engineered near the 5′ terminus of the 3′UTR, about halfway between the 5′ terminus and 3′ terminus of the 3′UTR and/or near the 3′ terminus of the 3′UTR in a polynucleotide of the disclosure. As a non-limiting example, a miRNA binding site can be engineered near the 5′ terminus of the 3′UTR and about halfway between the 5′ terminus and 3′ terminus of the 3′UTR. As another non-limiting example, a miRNA binding site can be engineered near the 3′ terminus of the 3′UTR and about halfway between the 5′ terminus and 3′ terminus of the 3′UTR. As yet another non-limiting example, a miRNA binding site can be engineered near the 5′ terminus of the 3′UTR and near the 3′ terminus of the 3′UTR.

In another embodiment, a 3′UTR can comprise 1, 2, 3, 4, 5, 6, 7, 8, 9, or 10 miRNA binding sites. The miRNA binding sites can be complementary to a miRNA, miRNA seed sequence, and/or miRNA sequences flanking the seed sequence.

In one embodiment, a polynucleotide of the disclosure can be engineered to include more than one miRNA site expressed in different tissues or different cell types of a subject. As a non-limiting example, a polynucleotide of the disclosure can be engineered to include miR-192 and miR-122 to regulate expression of the polynucleotide in the liver and kidneys of a subject. In another embodiment, a polynucleotide of the disclosure can be engineered to include more than one miRNA site for the same tissue.

In some embodiments, the therapeutic window and or differential expression associated with the polypeptide encoded by a polynucleotide of the disclosure can be altered with a miRNA binding site. For example, a polynucleotide encoding a polypeptide that provides a death signal can be designed to be more highly expressed in cancer cells by virtue of the miRNA signature of those cells. Where a cancer cell expresses a lower level of a particular miRNA, the polynucleotide encoding the binding site for that miRNA (or miRNAs) would be more highly expressed. Hence, the polypeptide that provides a death signal triggers or induces cell death in the cancer cell. Neighboring noncancer cells, harboring a higher expression of the same miRNA would be less affected by the encoded death signal as the polynucleotide would be expressed at a lower level due to the effects of the miRNA binding to the binding site or “sensor” encoded in the 3′UTR. Conversely, cell survival or cytoprotective signals can be delivered to tissues containing cancer and non-cancerous cells where a miRNA has a higher expression in the cancer cells—the result being a lower survival signal to the cancer cell and a larger survival signal to the normal cell. Multiple polynucleotides can be designed and administered having different signals based on the use of miRNA binding sites as described herein.

In some embodiments, the expression of a polynucleotide of the disclosure can be controlled by incorporating at least one sensor sequence in the polynucleotide and formulating the polynucleotide for administration. As a non-limiting example, a polynucleotide of the disclosure can be targeted to a tissue or cell by incorporating a miRNA binding site and formulating the polynucleotide in a lipid nanoparticle comprising a cationic lipid, including any of the lipids described herein.

A polynucleotide of the disclosure can be engineered for more targeted expression in specific tissues, cell types, or biological conditions based on the expression patterns of miRNAs in the different tissues, cell types, or biological conditions. Through introduction of tissue-specific miRNA binding sites, a polynucleotide of the disclosure can be designed for optimal protein expression in a tissue or cell, or in the context of a biological condition.

In some embodiments, a polynucleotide of the disclosure can be designed to incorporate miRNA binding sites that either have 100% identity to known miRNA seed sequences or have less than 100% identity to miRNA seed sequences. In some embodiments, a polynucleotide of the disclosure can be designed to incorporate miRNA binding sites that have at least: 60%, 65%, 70%, 75%, 80%, 85%, 90%, 95%, 96%, 97%, 98%, or 99% identity to known miRNA seed sequences. The miRNA seed sequence can be partially mutated to decrease miRNA binding affinity and as such result in reduced downmodulation of the polynucleotide. In essence, the degree of match or mis-match between the miRNA binding site and the miRNA seed can act as a rheostat to more finely tune the ability of the miRNA to modulate protein expression. In addition, mutation in the non-seed region of a miRNA binding site can also impact the ability of a miRNA to modulate protein expression.

In one embodiment, a miRNA sequence can be incorporated into the loop of a stem loop.

In another embodiment, a miRNA seed sequence can be incorporated in the loop of a stem loop and a miRNA binding site can be incorporated into the 5′ or 3′ stem of the stem loop.

In one embodiment, a translation enhancer element (TEE) can be incorporated on the 5′end of the stem of a stem loop and a miRNA seed can be incorporated into the stem of the stem loop. In another embodiment, a TEE can be incorporated on the 5′ end of the stem of a stem loop, a miRNA seed can be incorporated into the stem of the stem loop and a miRNA binding site can be incorporated into the 3′ end of the stem or the sequence after the stem loop. The miRNA seed and the miRNA binding site can be for the same and/or different miRNA sequences.

In one embodiment, the incorporation of a miRNA sequence and/or a TEE sequence changes the shape of the stem loop region which can increase and/or decrease translation. (see e.g, Kedde et al., “A Pumilio-induced RNA structure switch in p27-3′UTR controls miR-221 and miR-22 accessibility.” Nature Cell Biology. 2010, incorporated herein by reference in its entirety).

In one embodiment, the 5′-UTR of a polynucleotide of the disclosure can comprise at least one miRNA sequence. The miRNA sequence can be, but is not limited to, a 19 or 22 nucleotide sequence and/or a miRNA sequence without the seed.

In one embodiment the miRNA sequence in the 5′UTR can be used to stabilize a polynucleotide of the disclosure described herein.

In another embodiment, a miRNA sequence in the 5′UTR of a polynucleotide of the disclosure can be used to decrease the accessibility of the site of translation initiation such as, but not limited to a start codon. See, e.g., Matsuda et al., PLOS One. 2010 11 (5): e15057; incorporated herein by reference in its entirety, which used antisense locked nucleic acid (LNA) oligonucleotides and exon-junction complexes (EJCs) around a start codon (−4 to +37 where the A of the AUG codons is +1) in order to decrease the accessibility to the first start codon (AUG). Matsuda showed that altering the sequence around the start codon with an LNA or EJC affected the efficiency, length and structural stability of a polynucleotide. A polynucleotide of the disclosure can comprise a miRNA sequence, instead of the LNA or EJC sequence described by Matsuda et al, near the site of translation initiation in order to decrease the accessibility to the site of translation initiation. The site of translation initiation can be prior to, after or within the miRNA sequence. As a non-limiting example, the site of translation initiation can be located within a miRNA sequence such as a seed sequence or binding site. As another non-limiting example, the site of translation initiation can be located within a miR-122 sequence such as the seed sequence or the mir-122 binding site.

In some embodiments, a polynucleotide of the disclosure can include at least one miRNA in order to dampen the antigen presentation by antigen presenting cells. The miRNA can be the complete miRNA sequence, the miRNA seed sequence, the miRNA sequence without the seed, or a combination thereof. As a non-limiting example, a miRNA incorporated into a polynucleotide of the disclosure can be specific to the hematopoietic system. As another non-limiting example, a miRNA incorporated into a polynucleotide of the disclosure to dampen antigen presentation is miR-142-3p.

In some embodiments, a polynucleotide of the disclosure can include at least one miRNA in order to dampen expression of the encoded polypeptide in a tissue or cell of interest. As a non-limiting example, a polynucleotide of the disclosure can include at least one miR-122 binding site in order to dampen expression of an encoded polypeptide of interest in the liver. As another non-limiting example a polynucleotide of the disclosure can include at least one miR-142-3p binding site, miR-142-3p seed sequence, miR-142-3p binding site without the seed, miR-142-5p binding site, miR-142-5p seed sequence, miR-142-5p binding site without the seed, miR-146 binding site, miR-146 seed sequence and/or miR-146 binding site without the seed sequence.

In some embodiments, a polynucleotide of the disclosure can comprise at least one miRNA binding site in the 3′UTR in order to selectively degrade mRNA therapeutics in the immune cells to subdue unwanted immunogenic reactions caused by therapeutic delivery. As a non-limiting example, the miRNA binding site can make a polynucleotide of the disclosure more unstable in antigen presenting cells. Non-limiting examples of these miRNAs include mir-142-5p, mir-142-3p, mir-146a-5p, and mir-146-3p.

In one embodiment, a polynucleotide of the disclosure comprises at least one miRNA sequence in a region of the polynucleotide that can interact with a RNA binding protein.

In some embodiments, the polynucleotide of the disclosure (e.g., a RNA, e.g., a mRNA) comprising (i) a sequence-optimized nucleotide sequence (e.g., an ORF) and (ii) a miRNA binding site (e.g., a miRNA binding site that binds to miR-142).

In some embodiments, the polynucleotide of the disclosure comprises a uracil-modified sequence encoding a polypeptide disclosed herein and a miRNA binding site disclosed herein, e.g., a miRNA binding site that binds to miR-142. In some embodiments, the uracil-modified sequence encoding a polypeptide comprises at least one chemically modified nucleobase, e.g., 5-methoxyuracil. In some embodiments, at least 95% of a type of nucleobase (e.g., uracil) in a uracil-modified sequence encoding a polypeptide of the disclosure are modified nucleobases. In some embodiments, at least 95% of uricil in a uracil-modified sequence encoding a polypeptide is 5-methoxyuridine. In some embodiments, the polynucleotide comprising a nucleotide sequence encoding a polypeptide disclosed herein and a miRNA binding site is formulated with a delivery agent, e.g., a compound having the Formula (I), e.g., any of Compounds 1-147.

### Modified Polynucleotides Comprising Functional RNA Elements

The present disclosure provides synthetic polynucleotides comprising a modification (e.g., an RNA element), wherein the modification provides a desired translational regulatory activity. In some embodiments, the disclosure provides a polynucleotide comprising a 5′ untranslated region (UTR), an initiation codon, a full open reading frame encoding a polypeptide, a 3′ UTR, and at least one modification, wherein the at least one modification provides a desired translational regulatory activity, for example, a modification that promotes and/or enhances the translational fidelity of mRNA translation. In some embodiments, the desired translational regulatory activity is a cis-acting regulatory activity. In some embodiments, the desired translational regulatory activity is an increase in the residence time of the 43S pre-initiation complex (PIC) or ribosome at, or proximal to, the initiation codon. In some embodiments, the desired translational regulatory activity is an increase in the initiation of polypeptide synthesis at or from the initiation codon. In some embodiments, the desired translational regulatory activity is an increase in the amount of polypeptide translated from the full open reading frame. In some embodiments, the desired translational regulatory activity is an increase in the fidelity of initiation codon decoding by the PIC or ribosome. In some embodiments, the desired translational regulatory activity is inhibition or reduction of leaky scanning by the PIC or ribosome. In some embodiments, the desired translational regulatory activity is a decrease in the rate of decoding the initiation codon by the PIC or ribosome. In some embodiments, the desired translational regulatory activity is inhibition or reduction in the initiation of polypeptide synthesis at any codon within the mRNA other than the initiation codon. In some embodiments, the desired translational regulatory activity is inhibition or reduction of the amount of polypeptide translated from any open reading frame within the mRNA other than the full open reading frame. In some embodiments, the desired translational regulatory activity is inhibition or reduction in the production of aberrant translation products. In some embodiments, the desired translational regulatory activity is a combination of one or more of the foregoing translational regulatory activities.

Accordingly, the present disclosure provides a polynucleotide, e.g., an mRNA, comprising an RNA element that comprises a sequence and/or an RNA secondary structure(s) that provides a desired translational regulatory activity as described herein. In some aspects, the mRNA comprises an RNA element that comprises a sequence and/or an RNA secondary structure(s) that promotes and/or enhances the translational fidelity of mRNA translation. In some aspects, the mRNA comprises an RNA element that comprises a sequence and/or an RNA secondary structure(s) that provides a desired translational regulatory activity, such as inhibiting and/or reducing leaky scanning. In some aspects, the disclosure provides an mRNA that comprises an RNA element that comprises a sequence and/or an RNA secondary structure(s) that inhibits and/or reduces leaky scanning thereby promoting the translational fidelity of the mRNA.

In some embodiments, the RNA element comprises natural and/or modified nucleotides. In some embodiments, the RNA element comprises of a sequence of linked nucleotides, or derivatives or analogs thereof, that provides a desired translational regulatory activity as described herein. In some embodiments, the RNA element comprises a sequence of linked nucleotides, or derivatives or analogs thereof, that forms or folds into a stable RNA secondary structure, wherein the RNA secondary structure provides a desired translational regulatory activity as described herein. RNA elements can be identified and/or characterized based on the primary sequence of the element (e.g., GC-rich element), by RNA secondary structure formed by the element (e.g. stem-loop), by the location of the element within the RNA molecule (e.g., located within the 5′ UTR of an mRNA), by the biological function and/or activity of the element (e.g., “translational enhancer element”), and any combination thereof.

In some aspects, the disclosure provides an mRNA having one or more structural modifications that inhibits leaky scanning and/or promotes the translational fidelity of mRNA translation, wherein at least one of the structural modifications is a GC-rich RNA element. In some aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is 70-80% cytosine, 60-70% cytosine, 50%-60% cytosine, 40-50% cytosine, 30-40% cytosine bases. In any of the foregoing or related aspects, the disclosure provides a GC-rich RNA element which comprises a sequence of 3-30, 5-25, 10-20, 15-20, about 20, about 15, about 12, about 10, about 7, about 6 or about 3 nucleotides, derivatives or analogs thereof, linked in any order, wherein the sequence composition is about 80% cytosine, about 70% cytosine, about 60% cytosine, about 50% cytosine, about 40% cytosine, or about 30% cytosine.

In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is 70-80% cytosine, 60-70% cytosine, 50%-60% cytosine, 40-50% cytosine, or 30-40% cytosine. In any of the foregoing or related aspects, the disclosure provides a GC-rich RNA element which comprises a sequence of 20, 19, 18, 17, 16, 15, 14, 13, 12, 11, 10, 9, 8, 7, 6, 5, 4, or 3 nucleotides, or derivatives or analogs thereof, linked in any order, wherein the sequence composition is about 80% cytosine, about 70% cytosine, about 60% cytosine, about 50% cytosine, about 40% cytosine, or about 30% cytosine.

In some embodiments, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of 3, 4, 5, 6, 7, 8, 9, 10, 11, 12, 13, 14, 15, 16, 17, 18, 19, or 20 nucleotides, or derivatives or analogs thereof, linked in any order, wherein the sequence composition is >50% cytosine. In some embodiments, the sequence composition is >55% cytosine, >60% cytosine, >65% cytosine, >70% cytosine, >75% cytosine, >80% cytosine, >85% cytosine, or >90% cytosine.

In other aspects, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA, and wherein the GC-rich RNA element comprises a sequence of about 3-30, 5-25, 10-20, 15-20 or about 20, about 15, about 12, about 10, about 6 or about 3 nucleotides, or derivatives or analogues thereof, wherein the sequence comprises a repeating GC-motif, wherein the repeating GC-motif is [CCG]n, wherein n=1 to 10, n=2 to 8, n=3 to 6, or n=4 to 5. In some embodiments, the sequence comprises a repeating GC-motif [CCG]n, wherein n=1, 2, 3, 4 or 5. In some embodiments, the sequence comprises a repeating GC-motif [CCG]n, wherein n=1, 2, or 3. In some embodiments, the sequence comprises a repeating GC-motif [CCG]n, wherein n=1. In some embodiments, the sequence comprises a repeating GC-motif [CCG]n, wherein n=2. In some embodiments, the sequence comprises a repeating GC-motif [CCG]n, wherein n=3. In some embodiments, the sequence comprises a repeating GC-motif [CCG]n, wherein n=4 (SEQ ID NO: 1384). In some embodiments, the sequence comprises a repeating GC-motif [CCG]n, wherein n=5 (SEQ ID NO: 1382).

In another aspect, the disclosure provides a modified mRNA comprising at least one modification, wherein at least one modification is a GC-rich RNA element comprising a sequence of linked nucleotides, or derivatives or analogs thereof, preceding a Kozak consensus sequence in a 5′ UTR of the mRNA, wherein the GC-rich RNA element comprises any one of the sequences set forth in Table 4. In one embodiment, the GC-rich RNA element is located about 30, about 25, about 20, about 15, about 10, about 5, about 4, about 3, about 2, or about 1 nucleotide(s) upstream of a Kozak consensus sequence in the 5′ UTR of the mRNA. In another embodiment, the GC-rich RNA element is located about 15-30, 15-20, 15-25, 10-15, or 5-10 nucleotides upstream of a Kozak consensus sequence. In another embodiment, the GC-rich RNA element is located immediately adjacent to a Kozak consensus sequence in the 5′ UTR of the mRNA.

In other aspects, the disclosure provides a modified mRNA comprising at least one modification, wherein at least one modification is a GC-rich RNA element comprising the sequence V1 [CCCCGGCGCC] (SEQ ID NO: 1383) as set forth in Table 4, or derivatives or analogs thereof, preceding a Kozak consensus sequence in the 5′ UTR of the mRNA. In some embodiments, the GC-rich element comprises the sequence V1 as set forth in Table 4 located immediately adjacent to and upstream of the Kozak consensus sequence in the 5′ UTR of the mRNA. In some embodiments, the GC-rich element comprises the sequence V1 as set forth in Table 4 located 1, 2, 3, 4, 5, 6, 7, 8, 9 or 10 bases upstream of the Kozak consensus sequence in the 5′ UTR of the mRNA. In other embodiments, the GC-rich element comprises the sequence V1 as set forth in Table 4 located 1-3, 3-5, 5-7, 7-9, 9-12, or 12-15 bases upstream of the Kozak consensus sequence in the 5′ UTR of the mRNA.

In other aspects, the disclosure provides a modified mRNA comprising at least one modification, wherein at least one modification is a GC-rich RNA element comprising the sequence V2 [CCCCGGC] as set forth in Table 4, or derivatives or analogs thereof, preceding a Kozak consensus sequence in the 5′ UTR of the mRNA. In some embodiments, the GC-rich element comprises the sequence V2 as set forth in Table 4 located immediately adjacent to and upstream of the Kozak consensus sequence in the 5′ UTR of the mRNA. In some embodiments, the GC-rich element comprises the sequence V2 as set forth in Table 4 located 1, 2, 3, 4, 5, 6, 7, 8, 9 or 10 bases upstream of the Kozak consensus sequence in the 5′ UTR of the mRNA. In other embodiments, the GC-rich element comprises the sequence V2 as set forth in Table 4 located 1-3, 3-5, 5-7, 7-9, 9-12, or 12-15 bases upstream of the Kozak consensus sequence in the 5′ UTR of the mRNA.

In other aspects, the disclosure provides a modified mRNA comprising at least one modification, wherein at least one modification is a GC-rich RNA element comprising the sequence EK [GCCGCC] as set forth in Table 4, or derivatives or analogs thereof, preceding a Kozak consensus sequence in the 5′ UTR of the mRNA. In some embodiments, the GC-rich element comprises the sequence EK as set forth in Table 4 located immediately adjacent to and upstream of the Kozak consensus sequence in the 5′ UTR of the mRNA. In some embodiments, the GC-rich element comprises the sequence EK as set forth in Table 4 located 1, 2, 3, 4, 5, 6, 7, 8, 9 or 10 bases upstream of the Kozak consensus sequence in the 5′ UTR of the mRNA. In other embodiments, the GC-rich element comprises the sequence EK as set forth in Table 4 located 1-3, 3-5, 5-7, 7-9, 9-12, or 12-15 bases upstream of the Kozak consensus sequence in the 5′ UTR of the mRNA.

In yet other aspects, the disclosure provides a modified mRNA comprising at least one modification, wherein at least one modification is a GC-rich RNA element comprising the sequence V1 [CCCCGGCGCC] (SEQ ID NO: 1383) as set forth in Table 4, or derivatives or analogs thereof, preceding a Kozak consensus sequence in the 5′ UTR of the mRNA, wherein the 5′ UTR comprises the following sequence shown in Table 4:

(SEQ ID NO: 1384)
GGGAAATAAGAGAGAAAAGAAGAGTAAGAAGAAATATAAGA.

In some embodiments, the GC-rich element comprises the sequence V1 as set forth in Table 4 located immediately adjacent to and upstream of the Kozak consensus sequence in the 5′ UTR sequence shown in Table 4. In some embodiments, the GC-rich element comprises the sequence V1 as set forth in Table 4 located 1, 2, 3, 4, 5, 6, 7, 8, 9 or 10 bases upstream of the Kozak consensus sequence in the 5′ UTR of the mRNA, wherein the 5′ UTR comprises the following sequence shown in Table 4:

(SEQ ID NO: 1384)
GGGAAATAAGAGAGAAAAGAAGAGTAAGAAGAAATATAAGA.

In other embodiments, the GC-rich element comprises the sequence V1 as set forth in Table 4 located 1-3, 3-5, 5-7, 7-9, 9-12, or 12-15 bases upstream of the Kozak consensus sequence in the 5′ UTR of the mRNA, wherein the 5′ UTR comprises the following sequence shown in Table 4:

(SEQ ID NO: 1384)
GGGAAATAAGAGAGAAAAGAAGAGTAAGAAGAAATATAAGA.

In some embodiments, the 5′ UTR comprises the following sequence set forth in Table 4:

(SEQ ID NO: 1385)
GGGAAATAAGAGAGAAAAGAAGAGTAAGAAGAAATATAAGACCCCGGCGCC
GCCACC

In some embodiments, the 5′ UTR comprises the following sequence set forth in Table 4:

(SEQ ID NO: 1386)
GGGAAATAAGAGAGAAAAGAAGAGTAAGAAGAAATATAAGACCCCGGCGCC
ACC

TABLE 4

| SEQ | | |
|---|---|---|
| ID | | |
| NO: | 5′ UTRs | 5′UTR Sequence |
| 1380 | Standard | GGGAAATAAGAGAGAAAAGAAGAG |
| | | TAAGAAGAAATATAAGAGCCACC |
| 1384 | UTR | GGGAAATAAGAGAGAAAAGAAGAG |
| | | TAAGAAGAAATATAAGA |
| 1385 | V1-UTR | GGGAAATAAGAGAGAAAAGAAGAG |
| | | TAAGAAGAAATATAAGACCCCGGC |
| | | GCCGCCACC |
| 1386 | V2-UTR | GGGAAATAAGAGAGAAAAGAAGAG |
| | | TAAGAAGAAATATAAGACCCCGGC |
| | | GCCACC |
| | GC-Rich RNA Elements | Sequence |
| | K0 (Traditional | [GCCA/GCC] |
| | Kozak consensus) | |
| | EK | [GCCGCC] |
| 1383 | V1 | [CCCCGGCGCC] |
| | V2 | [CCCCGGC] |
| | (CCG)ₙ, | [CCG]ₙ |
| | where n = 1-10 | |
| | (GCC)ₙ, | [GCC]ₙ |
| | where n = 1-10 | |
| 1381 | (CCG)ₙ, | [CCGCCGCCGCCG] |
| | where n = 4 | |
| 1382 | (CCG)ₙ, | [CCGCCGCCGCCGCCG] |
| | where n = 5 | |

In another aspect, the disclosure provides a modified mRNA comprising at least one modification, wherein at least one modification is a GC-rich RNA element comprising a stable RNA secondary structure comprising a sequence of nucleotides, or derivatives or analogs thereof, linked in an order which forms a hairpin or a stem-loop. In one embodiment, the stable RNA secondary structure is upstream of the Kozak consensus sequence. In another embodiment, the stable RNA secondary structure is located about 30, about 25, about 20, about 15, about 10, or about 5 nucleotides upstream of the Kozak consensus sequence. In another embodiment, the stable RNA secondary structure is located about 20, about 15, about 10 or about 5 nucleotides upstream of the Kozak consensus sequence. In another embodiment, the stable RNA secondary structure is located about 5, about 4, about 3, about 2, about 1 nucleotides upstream of the Kozak consensus sequence. In another embodiment, the stable RNA secondary structure is located about 15-30, about 15-20, about 15-25, about 10-15, or about 5-10 nucleotides upstream of the Kozak consensus sequence. In another embodiment, the stable RNA secondary structure is located 12-15 nucleotides upstream of the Kozak consensus sequence. In another embodiment, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In another embodiment, the modification is operably linked to an open reading frame encoding a polypeptide and wherein the modification and the open reading frame are heterologous.

In another embodiment, the sequence of the GC-rich RNA element is comprised exclusively of guanine (G) and cytosine (C) nucleobases.

RNA elements that provide a desired translational regulatory activity as described herein can be identified and characterized using known techniques, such as ribosome profiling. Ribosome profiling is a technique that allows the determination of the positions of PICs and/or ribosomes bound to mRNAs (see e.g., Ingolia et al., (2009) Science 324(5924):218-23, incorporated herein by reference). The technique is based on protecting a region or segment of mRNA, by the PIC and/or ribosome, from nuclease digestion. Protection results in the generation of a 30-bp fragment of RNA termed a ‘footprint’. The sequence and frequency of RNA footprints can be analyzed by methods known in the art (e.g., RNA-seq). The footprint is roughly centered on the A-site of the ribosome. If the PIC or ribosome dwells at a particular position or location along an mRNA, footprints generated at these position would be relatively common. Studies have shown that more footprints are generated at positions where the PIC and/or ribosome exhibits decreased processivity and fewer footprints where the PIC and/or ribosome exhibits increased processivity (Gardin et al., (2014) eLife 3:e03735). In some embodiments, residence time or the time of occupancy of a the PIC or ribosome at a discrete position or location along an polynucleotide comprising any one or more of the RNA elements described herein is determined by ribosome profiling.

### Delivery Vehicles

### General

The mRNAs of the disclosure may be formulated in nanoparticles or other delivery vehicles, e.g., to protect them from degradation when delivered to a subject. Illustrative nanoparticles are described in Panyam, J. & Labhasetwar, V. Adv. Drug Deliv. Rev. 55, 329-347 (2003) and Peer, D. et al. Nature Nanotech. 2, 751-760 (2007). In certain embodiments, an mRNA of the disclosure is encapsulated within a nanoparticle. In particular embodiments, a nanoparticle is a particle having at least one dimension (e.g., a diameter) less than or equal to 1000 nM, less than or equal to 500 nM or less than or equal to 100 nM. In particular embodiments, a nanoparticle includes a lipid. Lipid nanoparticles include, but are not limited to, liposomes and micelles. Any of a number of lipids may be present, including cationic and/or ionizable lipids, anionic lipids, neutral lipids, amphipathic lipids, PEGylated lipids, and/or structural lipids. Such lipids can be used alone or in combination. In particular embodiments, a lipid nanoparticle comprises one or more mRNAs described herein.

In some embodiments, the lipid nanoparticle formulations of the mRNAs described herein may include one or more (e.g., 1, 2, 3, 4, 5, 6, 7, or 8) cationic and/or ionizable lipids. Such cationic and/or ionizable lipids include, but are not limited to, 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), (2S)-2-({8-[(3B)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)).N,N-dioleyl-N,N-dimethylammonium chloride (“DODAC”); N-(2,3-dioleyloxy) propyl-N,N-N-triethylammonium chloride (“DOTMA”); N,N-distearyl-N,N-dimethylammonium bromide (“DDAB”); N-(2,3-dioleoyloxy) propyl)-N,N,N-trimethylammonium chloride (“DOTAP”); 1,2-Dioleyloxy-3-trimethylaminopropane chloride salt (“DOTAP.CI”); 3-β-(N—(N′,N′-dimethylaminoethane)-carbamoyl) cholesterol (“DC-Chol”), N-(1-(2,3-dioleyloxy) propyl)-N-2-(sperminecarboxamido)ethyl)-N,N-dimethyl-ammonium trifluoracetate (“DOSPA”), dioctadecylamidoglycyl carboxyspermine (“DOGS”), 1,2-dioleoyl-3-dimethylammonium propane (“DODAP”), N,N-dimethyl-2,3-dioleyloxy) propylamine (“DODMA”), and N-(1,2-dimyristyloxyprop-3-yl)-N,N-dimethyl-N-hydroxyethyl ammonium bromide (“DMRIE”). Additionally, a number of commercial preparations of cationic and/or ionizable lipids can be used, such as, e.g., LIPOFECTIN® (including DOTMA and DOPE, available from GIBCO/BRL), and LIPOFECTAMINE® (including DOSPA and DOPE, available from GIBCO/BRL). KL10, KL22, and KL25 are described, for example, in U.S. Pat. No. 8,691,750, which is incorporated herein by reference in its entirety. In particular embodiments, the lipid is DLin-MC3-DMA or DLin-KC2-DMA.

Anionic lipids suitable for use in lipid nanoparticles of the disclosure include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids.

Neutral lipids suitable for use in lipid nanoparticles of the disclosure include, but are not limited to, diacylphosphatidylcholine, diacylphosphatidylethanolamine, ceramide, sphingomyelin, dihydrosphingomyelin, cephalin, and cerebrosides. Lipids having a variety of acyl chain groups of varying chain length and degree of saturation are available or may be isolated or synthesized by well-known techniques. Additionally, lipids having mixtures of saturated and unsaturated fatty acid chains can be used. In some embodiments, the neutral lipids used in the disclosure are DOPE, DSPC, DPPC, POPC, or any related phosphatidylcholine. In some embodiments, the neutral lipid may be composed of sphingomyelin, dihydrosphingomyeline, or phospholipids with other head groups, such as serine and inositol.

In some embodiments, amphipathic lipids are included in nanoparticles of the disclosure. Exemplary amphipathic lipids suitable for use in nanoparticles of the disclosure include, but are not limited to, sphingolipids, phospholipids, and aminolipids. In some embodiments, a phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine,1,2-dioleoyl-sn-glycero-3-phosphoetha nolamine (DOPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. Other phosphorus-lacking compounds, such as sphingolipids, glycosphingolipid families, diacylglycerols, and β-acyloxyacids, may also be used. Additionally, such amphipathic lipids can be readily mixed with other lipids, such as triglycerides and sterols.

In some embodiments, the lipid component of a nanoparticle of the disclosure may include one or more PEGylated lipids. A PEGylated lipid (also known as a PEG lipid or a PEG-modified lipid) is a lipid modified with polyethylene glycol. The lipid component may include one or more PEGylated lipids. A PEGylated lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, and PEG-modified dialkylglycerols. For example, a PEGylated lipid may be PEG-c-DOMG, PEG-DMG, PEG-DLPE, PEG-DMPE, PEG-DPPC, or a PEG-DSPE lipid.

A lipid nanoparticle of the disclosure may include one or more structural lipids. Exemplary, non-limiting structural lipids that may be present in the lipid nanoparticles of the disclosure include cholesterol, fecosterol, sitosterol, campesterol, stigmasterol, brassicasterol, ergosterol, tomatidine, tomatine, ursolic acid, or alpha-tocopherol.

In some embodiments, one or more mRNA of the disclosure may be formulated in a lipid nanoparticle having a diameter from about 1 nm to about 900 nm, e.g., about 1 nm to about 100 nm, about 1 nm to about 200 nm, about 1 nm to about 300 nm, about 1 nm to about 400 nm, about 1 nm to about 500 nm, about 1 nm to about 600 nm, about 1 nm to about 700 nm, about 1 nm to 800 nm, about 1 nm to about 900 nm. In some embodiments, the nanoparticle may have a diameter from about 10 nm to about 300 nm, about 20 nm to about 200 nm, about 30 nm to about 100 nm, or about 40 nm to about 80 nm. In some embodiments, the nanoparticle may have a diameter from about 30 nm to about 300 nm, about 40 nm to about 200 nm, about 50 nm to about 150 nm, about 70 to about 110 nm, or about 80 nm to about 120 nm. In one embodiment, an mRNA may be formulated in a lipid nanoparticle having a diameter from about 10 to about 100 nm including ranges in between such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm, and/or about 90 to about 100 nm. In one embodiment, an mRNA may be formulated in a lipid nanoparticle having a diameter from about 30 nm to about 300 nm, about 40 nm to about 200 nm, about 50 nm to about 150 nm, about 70 to about 110 nm, or about 80 nm to about 120 nm including ranges in between.

In some embodiments, a lipid nanoparticle may have a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, or greater than 950 nm.

In some embodiments, the particle size of the lipid nanoparticle may be increased and/or decreased. The change in particle size may be able to help counter a biological reaction such as, but not limited to, inflammation, or may increase the biological effect of the mRNA delivered to a patient or subject.

In certain embodiments, it is desirable to target a nanoparticle, e.g., a lipid nanoparticle, of the disclosure using a targeting moiety that is specific to a cell type and/or tissue type. In some embodiments, a nanoparticle may be targeted to a particular cell, tissue, and/or organ using a targeting moiety. In particular embodiments, a nanoparticle comprises one or more mRNA described herein and a targeting moiety. Exemplary non-limiting targeting moieties include ligands, cell surface receptors, glycoproteins, vitamins (e.g., riboflavin) and antibodies (e.g., full-length antibodies, antibody fragments (e.g., Fv fragments, single chain Fv (scFv) fragments, Fab′ fragments, or F(ab′)2 fragments), single domain antibodies, camelid antibodies and fragments thereof, human antibodies and fragments thereof, monoclonal antibodies, and multispecific antibodies (e.g. bispecific antibodies)). In some embodiments, the targeting moiety may be a polypeptide. The targeting moiety may include the entire polypeptide (e.g., peptide or protein) or fragments thereof. A targeting moiety is typically positioned on the outer surface of the nanoparticle in such a manner that the targeting moiety is available for interaction with the target, for example, a cell surface receptor. A variety of different targeting moieties and methods are known and available in the art, including those described, e.g., in Sapra et al., Prog. Lipid Res. 42(5):439-62, 2003 and Abra et al., J. Liposome Res. 12:1-3, 2002.

In some embodiments, a lipid nanoparticle (e.g., a liposome) may include a surface coating of hydrophilic polymer chains, such as polyethylene glycol (PEG) chains (see, e.g., Allen et al., Biochimica et Biophysica Acta 1237:99-108, 1995; DeFrees et al., Journal of the American Chemistry Society 118:6101-6104, 1996; Blume et al., Biochimica et Biophysica Acta 1149:180-184,1993; Klibanov et al., Journal of Liposome Research 2:321-334, 1992; U.S. Pat. No. 5,013,556; Zalipsky, Bioconjugate Chemistry 4:296-299, 1993; Zalipsky, FEBS Letters 353:71-74, 1994; Zalipsky, in Stealth Liposomes Chapter 9 (Lasic and Martin, Eds) CRC Press, Boca Raton Fla., 1995). In one approach, a targeting moiety for targeting the lipid nanoparticle is linked to the polar head group of lipids forming the nanoparticle. In another approach, the targeting moiety is attached to the distal ends of the PEG chains forming the hydrophilic polymer coating (see, e.g., Klibanov et al., Journal of Liposome Research 2:321-334, 1992; Kirpotin et al., FEBS Letters 388:115-118, 1996).

Standard methods for coupling the targeting moiety or moieties may be used. For example, phosphatidylethanolamine, which can be activated for attachment of targeting moieties, or derivatized lipophilic compounds, such as lipid-derivatized bleomycin, can be used. Antibody-targeted liposomes can be constructed using, for instance, liposomes that incorporate protein A (see, e.g., Renneisen et al., J. Bio. Chem., 265:16337-16342, 1990 and Leonetti et al., Proc. Natl. Acad. Sci. (USA), 87:2448-2451, 1990). Other examples of antibody conjugation are disclosed in U.S. Pat. No. 6,027,726. Examples of targeting moieties can also include other polypeptides that are specific to cellular components, including antigens associated with neoplasms or tumors. Polypeptides used as targeting moieties can be attached to the liposomes via covalent bonds (see, for example Heath, Covalent Attachment of Proteins to Liposomes, 149 Methods in Enzymology 111-119 (Academic Press, Inc. 1987)). Other targeting methods include the biotin-avidin system.

In some embodiments, a lipid nanoparticle of the disclosure includes a targeting moiety that targets the lipid nanoparticle to a cell including, but not limited to, hepatocytes, colon cells, epithelial cells, hematopoietic cells, epithelial cells, endothelial cells, lung cells, bone cells, stem cells, mesenchymal cells, neural cells, cardiac cells, adipocytes, vascular smooth muscle cells, cardiomyocytes, skeletal muscle cells, beta cells, pituitary cells, synovial lining cells, ovarian cells, testicular cells, fibroblasts, B cells, T cells, reticulocytes, leukocytes, granulocytes, and tumor cells (including primary tumor cells and metastatic tumor cells). In particular embodiments, the targeting moiety targets the lipid nanoparticle to a hepatocyte. In other embodiments, the targeting moiety targets the lipid nanoparticle to a colon cell. In some embodiments, the targeting moiety targets the lipid nanoparticle to a liver cancer cell (e.g., a hepatocellular carcinoma cell) or a colorectal cancer cell (e.g., a primary tumor or a metastasis).

### Lipid Nanoparticles

In one set of embodiments, lipid nanoparticles (LNPs) are provided. In one embodiment, a lipid nanoparticle comprises lipids including an ionizable lipid, a structural lipid, a phospholipid, and one or more mRNAs. Each of the LNPs described herein may be used as a formulation for the mRNA described herein. In one embodiment, a lipid nanoparticle comprises an ionizable lipid, a structural lipid, a phospholipid, a PEG-modified lipid and one or more mRNAs. In some embodiments, the LNP comprises an ionizable lipid, a PEG-modified lipid, a sterol and a phospholipid. In some embodiments, the LNP has a molar ratio of about 20-60% ionizable lipid: about 5-25% phospholipid: about 25-55% sterol; and about 0.5-15% PEG-modified lipid. In some embodiments, the LNP comprises a molar ratio of about 50% ionizable lipid, about 1.5% PEG-modified lipid, about 38.5% cholesterol and about 10% phospholipid. In some embodiments, the LNP comprises a molar ratio of about 55% ionizable lipid, about 2.5% PEG lipid, about 32.5% cholesterol and about 10% phospholipid. In some embodiments, the ionizable lipid is an ionizable amino or cationic lipid and the neutral lipid is a phospholipid, and the sterol is a cholesterol. In some embodiments, the LNP has a molar ratio of 50:38.5:10:1.5 of ionizable lipid: cholesterol: DSPC (1,2-dioctadecanoyl-sn-glycero-3-phosphocholine): PEG-DMG.

a. Ionizable Lipid

The present disclosure provides pharmaceutical compositions with advantageous properties. For example, the lipids described herein (e.g. those having any of Formula (I), (IA), (II), (IIa), (IIb), (IIc), (IId), (IIe), (III), (IV), (V), or (VI) may be advantageously used in lipid nanoparticle compositions for the delivery of therapeutic and/or prophylactic agents to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent has an increased therapeutic index as compared to a corresponding formulation which comprises a reference lipid (e.g., MC3, KC2, or DLinDMA) and the same therapeutic or prophylactic agent. In particular, the present application provides pharmaceutical compositions comprising:
(a) a polynucleotide comprising a nucleotide sequence encoding a polypeptide of interest; and
(b) a delivery agent.

In some embodiments, the delivery agent comprises a lipid compound having the Formula (I)

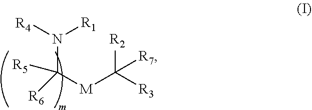

wherein
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁-14 alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, (CH₂)ₙCHQR, -CHQR, -CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, —N(R)R₈, —O(CH₂)ₙOR, —N(R)C(═NR₉) N(R)₂, —N(R)C(═CHR₉) N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉) N(R)₂, —N(OR)C(═CHR₉) N(R)₂, —C(═NR₉)N(R)₂, —C(═NR₉)R, —C(O)N(R)OR, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and m is selected from 5, 6, 7, 8, 9, 10, 11, 12, and 13,
or salts or stereoisomers thereof.

In some embodiments, a subset of compounds of Formula (I) includes those in which
R₁ is selected from the group consisting of C₅₋₂₀ alkyl, C₅₋₂₀ alkenyl, —R*YR″, —YR″, and —R″M′R′;
R₂ and R₃ are independently selected from the group consisting of H, C₁-14 alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, (CH₂)ₙCHQR, -CHQR, -CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and m is selected from 5, 6, 7, 8, 9, 10, 11, 12, and 13,
or salts or stereoisomers thereof, wherein alkyl and alkenyl groups may be linear or branched.

In some embodiments, a subset of compounds of Formula (I) includes those in which when R₄ is —(CH₂)ₙQ, —(CH₂)ₙCHQR, -CHQR, or -CQ(R)₂, then (i) Q is not —N(R)₂ when n is 1, 2, 3, 4 or 5, or (ii) Q is not 5, 6, or 7-membered heterocycloalkyl when n is 1 or 2.

In another embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁-14 alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, (CH₂)ₙCHQR, -CHQR, -CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉) N(R)₂, —N(R)C(═CHR₉) N(R)₂, —O C(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉) N(R)₂, —N(OR)C(═CHR₉) N(R)₂, —C(═NR₉) N(R)₂, —C (═NR₉)R, —C(O)N(R) OR, and a 5- to 14-membered heterocycloalkyl having one or more heteroatoms selected from N, O, and S which is substituted with one or more substituents selected from oxo (═O), OH, amino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I;

and
m is selected from 5, 6, 7, 8, 9, 10, 11, 12, and 13,
or salts or stereoisomers thereof.

In another embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁-14 alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, (CH₂)ₙCHQR, -CHQR, -CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof.

In yet another embodiments, another subset of compounds of Formula (I) includes those in which
R₁ is selected from the group consisting of C₅₋₂₀ alkyl, C₅₋₂₀ alkenyl, —R*YR″, —YR″, and —R″M′R′;
R₂ and R₃ are independently selected from the group consisting of H, C₁-14 alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, (CH₂)ₙCHQR, -CHQR, -CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂) nN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉) N(R)₂, —N(R)C(═CHR₉) N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉) N(R)₂, —N(OR)C(═CHR₉) N(R)₂, —C(═NR₉)R, —C(O)N(R) OR, and —C(═NR₉) N(R)₂, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is -CHQR, and -CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof.

In yet another embodiments, another subset of compounds of Formula (I) includes those in which
R₁ is selected from the group consisting of C₅₋₂₀ alkyl, C₅₋₂₀ alkenyl, —R*YR″, —YR″, and —R″M′R′;
R₂ and R₃ are independently selected from the group consisting of H, C₁-14 alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, (CH₂)ₙCHQR, -CHQR, -CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and each n is independently selected from 1, 2, 3, 4, and 5; and when Q is a 5- to 14-membered heterocycle and (i) R₄ is —(CH₂)ₙQ in which n is 1 or 2, or (ii) R₄ is (CH₂)ₙCHQR in which n is 1, or (iii) R₄ is -CHQR, and -CQ(R)₂, then Q is either a 5- to 14-membered heteroaryl or 8- to 14-membered heterocycloalkyl;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof.

In still another embodiments, another subset of compounds of Formula (I) includes those in which R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₁-14 alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, (CH₂)ₙCHQR, -CHQR, -CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, —N(R)R₈, —O(CH₂)ₙOR, —N(R)C(═NR₉) N(R)₂, —N(R)C(═CHR₉) N(R)₂, —OC(O)N(R)₂, —N(R)C(O)OR, —N(OR)C(O)R, —N(OR)S(O)₂R, —N(OR)C(O)OR, —N(OR)C(O)N(R)₂, —N(OR)C(S)N(R)₂, —N(OR)C(═NR₉) N(R)₂, —N(OR)C(═CHR₉) N(R)₂, —C(═NR₉)R, —C(O)N(R) OR, and —C(═NR₉) N(R)₂, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
R₈ is selected from the group consisting of C₃₋₆ carbocycle and heterocycle;
R₉ is selected from the group consisting of H, CN, NO₂, C₁₋₆ alkyl, —OR, —S(O)₂R, —S(O)₂N(R)₂, C₂₋₆ alkenyl, C₃₋₆ carbocycle and heterocycle;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof.

In still another embodiments, another subset of compounds of Formula (I) includes those in which
R₁ is selected from the group consisting of C₅₋₂₀ alkyl, C₅₋₂₀ alkenyl, —R*YR″, —YR″, and —R″M′R′;
R₂ and R₃ are independently selected from the group consisting of H, C₁-14 alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, (CH₂)ₙCHQR, -CHQR, -CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof.

In yet another embodiments, another subset of compounds of Formula (I) includes those in which
R₁ is selected from the group consisting of C₅₋₃₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof.

In yet another embodiments, another subset of compounds of Formula (I) includes those in which
R₁ is selected from the group consisting of C₅₋₂₀ alkyl, C₅₋₂₀ alkenyl, —R*YR″, —YR″, and —R″M′R′;
R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is —(CH₂)ₙQ or —(CH₂)ₙCHQR, where Q is —N(R)₂, and n is selected from 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof.

In still other embodiments, another subset of compounds of Formula (I) includes those in which R₁ is selected from the group consisting of C₅₋₃₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, -CHQR, and -CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, —S—S—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂-18 alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof.

In still other embodiments, another subset of compounds of Formula (I) includes those in which
R₁ is selected from the group consisting of C₅₋₂₀ alkyl, C₅₋₂₀ alkenyl, —R*YR″, —YR″, and —R″M′R′;
R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of —(CH₂)ₙQ, —(CH₂)ₙCHQR, -CHQR, and -CQ(R)₂, where Q is —N(R)₂, and n is selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂-18 alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₁₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof.

In certain embodiments, a subset of compounds of Formula (I) includes those of Formula (IA):

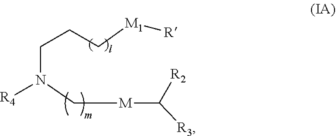

or a salt or stereoisomer thereof, wherein 1 is selected from 1, 2, 3, 4, and 5; m is selected from 5, 6, 7, 8, and 9; M1 is a bond or M′; R₄ is unsubstituted C₁₋₃ alkyl, or (CH₂)ₙQ, in which Q is OH, —NHC(S)N(R)₂, —NHC(O)N(R)₂, —N(R)C(O)R, —N(R)S(O)₂R, —N(R)R₈, —NHC(═NR₉) N(R)₂, —NHC(═CHR₉) N(R)₂, —OC(O)N(R)₂, —N(R)C(O)OR, heteroaryl, or heterocycloalkyl; M and M′ are independently selected from —C(O)O—, —OC(O)—, —C(O)N(R′)—, —P(O)(OR′)O—, —S—S—, an aryl group, and a heteroaryl group; and
R₂ and R₃ are independently selected from the group consisting of H, C₁-14 alkyl, and C₂₋₁₄ alkenyl.

In some embodiments, a subset of compounds of Formula (I) includes those of Formula (IA), or a salt or stereoisomer thereof,
wherein
1 is selected from 1, 2, 3, 4, and 5; m is selected from 5, 6, 7, 8, and 9;
M₁ is a bond or M′;
R₄ is unsubstituted C₁₋₃ alkyl, or —(CH₂)ₙQ, in which Q is OH, —NHC(S)N(R)₂, or —NHC(O)N(R)₂;
M and M′ are independently selected from —C(O)O—, —OC(O)—, —C(O)N(R′)—, —P(O)(OR′)O—, an aryl group, and a heteroaryl group; and
R₂ and R₃ are independently selected from the group consisting of H, C₁-14 alkyl, and C₂₋₁₄ alkenyl.

In certain embodiments, a subset of compounds of Formula (I) includes those of Formula (II):

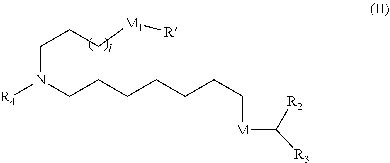

or a salt or stereoisomer thereof, wherein 1 is selected from 1, 2, 3, 4, and 5; M1 is a bond or M′; R₄ is unsubstituted C₁₋₃ alkyl, or —(CH₂)ₙQ, in which n is 2, 3, or 4, and Q is OH, —NHC(S)N(R)₂, —NHC(O)N(R)₂, —N(R)C(O)R, —N(R)S(O)₂R, —N(R)R₈, —NHC(═NR₉) N(R)₂, —NHC(═CHR₉) N(R)₂, —OC(O)N(R)₂, —N(R)C(O)OR, heteroaryl, or heterocycloalkyl; M and M′ are independently selected from —C(O)O—, —OC(O)—, —C(O)N(R′)—, —P(O)(OR′)O—, —S—S—, an aryl group, and a heteroaryl group; and
R₂ and R₃ are independently selected from the group consisting of H, C₁-14 alkyl, and C₂₋₁₄ alkenyl.

In some embodiments, a subset of compounds of Formula (I) includes those of Formula (II), or a salt or stereoisomer thereof, wherein
1 is selected from 1, 2, 3, 4, and 5;
M₁ is a bond or M′;
R₄ is unsubstituted C₁₋₃ alkyl, or —(CH₂)ₙQ, in which n is 2, 3, or 4, and Q is OH, —NHC(S)N(R)₂, or —NHC(O)N(R)₂;
M and M′ are independently selected from —C(O)O—, —OC(O)—, —C(O)N(R′)—, —P(O)(OR′)O—, an aryl group, and a heteroaryl group; and
R₂ and R₃ are independently selected from the group consisting of H, C₁-14 alkyl, and C₂₋₁₄ alkenyl.

In some embodiments, the compound of formula (I) is of the formula (IIa),

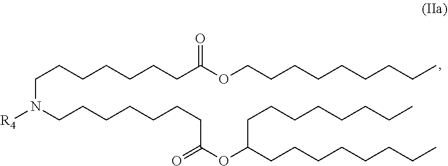

or a salt thereof, wherein R₄ is as described above.

In some embodiments, the compound of formula (I) is of the formula (IIb),

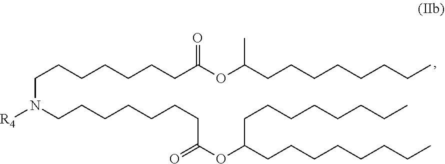

or a salt thereof, wherein R₄ is as described above.

In some embodiments, the compound of formula (I) is of the formula (IIc),

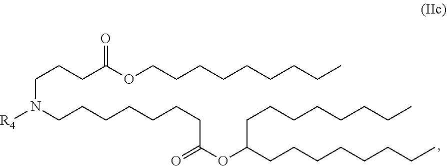

or a salt thereof, wherein R₄ is as described above.

In some embodiments, the compound of formula (I) is of the formula (IIe):

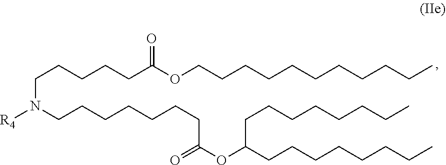

or a salt thereof, wherein R₄ is as described above.

In some embodiments, the compound of formula (IIa), (IIb), (IIc), or (IIe) comprises an R₄ which is selected from —(CH₂)ₙQ and —(CH₂)ₙCHQR, wherein Q, R and n are as defined above.

In some embodiments, Q is selected from the group consisting of —OR, —OH, —O(CH₂)_N(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle, wherein R is as defined above. In some aspects, n is 1 or 2. In some embodiments, Q is OH, —NHC(S)N(R)₂, or —NHC(O)N(R)₂.

In some embodiments, the compound of formula (I) is of the formula (IId),

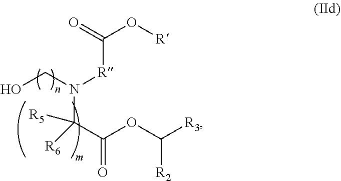

or a salt thereof, wherein R₂ and R₃ are independently selected from the group consisting of C₅₋₁₄ alkyl and C₅₋₁₄ alkenyl, n is selected from 2, 3, and 4, and R′, R″, R₅, R₆ and m are as defined above.

In some aspects of the compound of formula (IId), R₂ is C₈ alkyl. In some aspects of the compound of formula (IId), R₃ is C₅-C9 alkyl. In some aspects of the compound of formula (IId), m is 5, 7, or 9. In some aspects of the compound of formula (IId), each R₅ is H. In some aspects of the compound of formula (IId), each R₆ is H.

In another aspect, the present application provides a lipid composition (e.g., a lipid nanoparticle (LNP)) comprising: (1) a compound having the formula (I); (2) optionally a helper lipid (e.g. a phospholipid); (3) optionally a structural lipid (e.g. a sterol); and (4) optionally a lipid conjugate (e.g. a PEG-lipid). In exemplary embodiments, the lipid composition (e.g., LNP) further comprises a polynucleotide encoding a polypeptide of interest, e.g., a polynucleotide encapsulated therein.

As used herein, the term “alkyl” or “alkyl group” means a linear or branched, saturated hydrocarbon including one or more carbon atoms (e.g., one, two, three, four, five, six, seven, eight, nine, ten, eleven, twelve, thirteen, fourteen, fifteen, sixteen, seventeen, eighteen, nineteen, twenty, or more carbon atoms).

The notation “C₁₋₁₄ alkyl” means a linear or branched, saturated hydrocarbon including 1-14 carbon atoms. An alkyl group can be optionally substituted.

As used herein, the term “alkenyl” or “alkenyl group” means a linear or branched hydrocarbon including two or more carbon atoms (e.g., two, three, four, five, six, seven, eight, nine, ten, eleven, twelve, thirteen, fourteen, fifteen, sixteen, seventeen, eighteen, nineteen, twenty, or more carbon atoms) and at least one double bond.

The notation “C₂₋₁₄ alkenyl” means a linear or branched hydrocarbon including 2-14 carbon atoms and at least one double bond. An alkenyl group can include one, two, three, four, or more double bonds. For example, C₁₈ alkenyl can include one or more double bonds. A C18 alkenyl group including two double bonds can be a linoleyl group. An alkenyl group can be optionally substituted.

As used herein, the term “carbocycle” or “carbocyclic group” means a mono- or multi-cyclic system including one or more rings of carbon atoms. Rings can be three, four, five, six, seven, eight, nine, ten, eleven, twelve, thirteen, fourteen, or fifteen membered rings. The notation “C₃₋₆ carbocycle” means a carbocycle including a single ring having 3-6 carbon atoms. Carbocycles can include one or more double bonds and can be aromatic (e.g., aryl groups). Examples of carbocycles include cyclopropyl, cyclopentyl, cyclohexyl, phenyl, naphthyl, and 1,2-dihydronaphthyl groups. Carbocycles can be optionally substituted.

As used herein, the term “heterocycle” or “heterocyclic group” means a mono- or multi-cyclic system including one or more rings, where at least one ring includes at least one heteroatom. Heteroatoms can be, for example, nitrogen, oxygen, or sulfur atoms. Rings can be three, four, five, six, seven, eight, nine, ten, eleven, or twelve membered rings. Heterocycles can include one or more double bonds and can be aromatic (e.g., heteroaryl groups). Examples of heterocycles include imidazolyl, imidazolidinyl, oxazolyl, oxazolidinyl, thiazolyl, thiazolidinyl, pyrazolidinyl, pyrazolyl, isoxazolidinyl, isoxazolyl, isothiazolidinyl, isothiazolyl, morpholinyl, pyrrolyl, pyrrolidinyl, furyl, tetrahydrofuryl, thiophenyl, pyridinyl, piperidinyl, quinolyl, and isoquinolyl groups. Heterocycles can be optionally substituted.

As used herein, a “biodegradable group” is a group that can facilitate faster metabolism of a lipid in a subject. A biodegradable group can be, but is not limited to, —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group.

As used herein, an “aryl group” is a carbocyclic group including one or more aromatic rings. Examples of aryl groups include phenyl and naphthyl groups.

As used herein, a “heteroaryl group” is a heterocyclic group including one or more aromatic rings. Examples of heteroaryl groups include pyrrolyl, furyl, thiophenyl, imidazolyl, oxazolyl, and thiazolyl. Both aryl and heteroaryl groups can be optionally substituted. For example, M and M′ can be selected from the non-limiting group consisting of optionally substituted phenyl, oxazole, and thiazole. In the formulas herein, M and M′ can be independently selected from the list of biodegradable groups above.

Alkyl, alkenyl, and cyclyl (e.g., carbocyclyl and heterocyclyl) groups can be optionally substituted unless otherwise specified. Optional substituents can be selected from the group consisting of, but are not limited to, a halogen atom (e.g., a chloride, bromide, fluoride, or iodide group), a carboxylic acid (e.g., —C(O)OH), an alcohol (e.g., a hydroxyl, —OH), an ester (e.g., —C(O)OR or —OC(O)R), an aldehyde (e.g., —C(O)H), a carbonyl (e.g., —C(O)R, alternatively represented by C═O), an acyl halide (e.g., —C(O)X, in which X is a halide selected from bromide, fluoride, chloride, and iodide), a carbonate (e.g., —OC(O)OR), an alkoxy (e.g., —OR), an acetal (e.g., —C(OR)₂R″″, in which each OR are alkoxy groups that can be the same or different and R″″ is an alkyl or alkenyl group), a phosphate (e.g., P(O)₄³⁻), a thiol (e.g., —SH), a sulfoxide (e.g., —S(O) R), a sulfinic acid (e.g., —S(O)OH), a sulfonic acid (e.g., —S(O)₂OH), a thial (e.g., —C(S) H), a sulfate (e.g., S(O)₄₂—), a sulfonyl (e.g., —S(O)₂₋), an amide (e.g., —C(O)NR₂, or —N(R)C(O) R), an azido (e.g., —N3), a nitro (e.g., —NO₂), a cyano (e.g., —CN), an isocyano (e.g., —NC), an acyloxy (e.g., —OC(O) R), an amino (e.g., —NR₂, —NRH, or —NH₂), a carbamoyl (e.g., —OC(O)NR₂, —OC(O)NRH, or —OC(O)NH₂), a sulfonamide (e.g., —S(O)₂NR₂, —S(O)₂NRH, —S(O)₂NH₂, —N(R)S(O)₂R, —N(H)S(O)₂R, —N(R)S(O)₂H, or —N(H)S(O)₂H), an alkyl group, an alkenyl group, and a cyclyl (e.g., carbocyclyl or heterocyclyl) group.

In any of the preceding, R is an alkyl or alkenyl group, as defined herein. In some embodiments, the substituent groups themselves can be further substituted with, for example, one, two, three, four, five, or six substituents as defined herein. For example, a C₁₋₆ alkyl group can be further substituted with one, two, three, four, five, or six substituents as described herein.

The compounds of any one of formulae (I), (IA), (II), (IIa), (IIb), (IIc), (IId), and (Ile) include one or more of the following features when applicable.

In some embodiments, R₄ is selected from the group consisting of a C₃₋₆ carbocycle, —(CH₂)ₙQ, —(CH₂)ₙCHQR, -CHQR, and -CQ(R)₂, where Q is selected from a C₃₋₆ carbocycle, 5- to 14-membered aromatic or non-aromatic heterocycle having one or more heteroatoms selected from N, O, S, and P, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5.

In another embodiment, R₄ is selected from the group consisting of a C₃₋₆ carbocycle, —(CH₂)ₙQ, —(CH₂)ₙCHQR, -CHQR, and -CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —C(R)N(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, and each n is independently selected from 1, 2, 3, 4, and 5.

In another embodiment, R₄ is selected from the group consisting of a C₃₋₆ carbocycle, —(CH₂)ₙQ, —(CH₂)ₙCHQR, -CHQR, and -CQ(R)₂, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —C(R)N(R)₂C(O)OR, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is -CHQR, and -CQ(R)₂, then Q is either a 5- to 14-membered heteroaryl or 8- to 14-membered heterocycloalkyl.

In another embodiment, R₄ is selected from the group consisting of a C₃₋₆ carbocycle, —(CH₂)ₙQ, —(CH₂)ₙCHQR, -CHQR, and -CQ(R)₂, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —C(R)N(R)₂C(O)OR, and each n is independently selected from 1, 2, 3, 4, and 5. In another embodiment, R₄ is unsubstituted C₁₋₄ alkyl, e.g., unsubstituted methyl.

In certain embodiments, the disclosure provides a compound having the Formula (I), wherein R₄ is —(CH₂)ₙQ or —(CH₂)ₙCHQR, where Q is —N(R)₂, and n is selected from 3, 4, and 5.

In certain embodiments, the disclosure provides a compound having the Formula (I), wherein R₄ is selected from the group consisting of —(CH₂)ₙQ, —(CH₂)ₙCHQR, -CHQR, and -CQ(R)₂, where Q is —N(R)₂, and n is selected from 1, 2, 3, 4, and 5.

In certain embodiments, the disclosure provides a compound having the Formula (I), wherein R₂ and R₃ are independently selected from the group consisting of C₂₋₁₄ alkyl, C₂₋₁₄ alkenyl, —R*YR″, —YR″, and —R*OR″, or R₂ and R₃, together with the atom to which they are attached, form a heterocycle or carbocycle, and R₄ is —(CH₂)ₙQ or (CH₂)ₙCHQR, where Q is —N(R)₂, and n is selected from 3, 4, and 5.

In certain embodiments, R₂ and R₃ are independently selected from the group consisting of C₂₋₁₄ alkyl, C₂₋₁₄ alkenyl, —R*YR″, —YR″, and —R*OR″, or R₂ and R₃, together with the atom to which they are attached, form a heterocycle or carbocycle.

In some embodiments, R₁ is selected from the group consisting of C₅₋₂₀ alkyl and C₅₋₂₀ alkenyl.

In other embodiments, R₁ is selected from the group consisting of —R*YR″, —YR″, and —R″M′R′.

In certain embodiments, R₁ is selected from —R*YR″ and —YR″. In some embodiments, Y is a cyclopropyl group. In some embodiments, R* is C₈ alkyl or C₈ alkenyl. In certain embodiments, R″ is C₃₋₁₂ alkyl. For example, R″ can be C₃ alkyl. For example, R″ can be C₄₋₈ alkyl (e.g., C₄, C₅, C₆, C₇, or C₈ alkyl).

In some embodiments, R₁ is C₅₋₂₀ alkyl. In some embodiments, R₁ is C₆ alkyl. In some embodiments, R₁ is C₈ alkyl. In other embodiments, R₁ is C₉ alkyl. In certain embodiments, R₁ is C₁₄ alkyl. In other embodiments, R₁ is C₁₈ alkyl.

In some embodiments, R₁ is C₅₋₂₀ alkenyl. In certain embodiments, R₁ is C₁₈ alkenyl. In some embodiments, R₁ is linoleyl.

In certain embodiments, R₁ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl, or heptadeca-9-yl). In certain embodiments, R₁ is

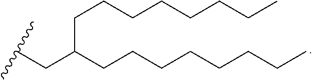 [SMILES: *CC(CCCCCCCC)CCCCCCCC]

In certain embodiments, R₁ is unsubstituted C₅₋₂₀ alkyl or C₅₋₂₀ alkenyl. In certain embodiments, R′ is substituted C₅₋₂₀ alkyl or C₅₋₂₀ alkenyl (e.g., substituted with a C₃₋₆ carbocycle such as 1-cyclopropylnonyl).

In other embodiments, R₁ is —R″M′R′.

In some embodiments, R′ is selected from —R*YR″ and —YR″. In some embodiments, Y is C₃₋₈ cycloalkyl. In some embodiments, Y is C₆₋₁₀ aryl. In some embodiments, Y is a cyclopropyl group. In some embodiments, Y is a cyclohexyl group. In certain embodiments, R* is C₁ alkyl.

In some embodiments, R″ is selected from the group consisting of C₃₋₁₂ alkyl and C₃₋₁₂ alkenyl. In some embodiments, R″ adjacent to Y is C₄ alkyl. In some embodiments, R″ adjacent to Y is C₄₋₉ alkyl (e.g., C₄, C₅, C₆, C₇ or C₈ or C₉ alkyl).

In some embodiments, R′ is selected from C₄ alkyl and C₄ alkenyl. In certain embodiments, R′ is selected from C₅ alkyl and C₅ alkenyl. In some embodiments, R′ is selected from C₆ alkyl and C₆ alkenyl. In some embodiments, R′ is selected from C₇ alkyl and C₇ alkenyl. In some embodiments, R′ is selected from C₉ alkyl and C₉ alkenyl.

In other embodiments, R′ is selected from C₁₁ alkyl and C₁₁ alkenyl. In other embodiments, R′ is selected from C₁₂ alkyl, C₁₂ alkenyl, C₁₃ alkyl, C₁₃ alkenyl, C₁₄ alkyl, C₁₄ alkenyl, C₁₅ alkyl, C₁₅ alkenyl, C₁₆ alkyl, C₁₆ alkenyl, C₁₇ alkyl, C₁₇ alkenyl, C₁₈ alkyl, and C₁₈ alkenyl. In certain embodiments, R′ is branched (e.g., decan-2-yl, undecan-3-yl, dodecan-4-yl, tridecan-5-yl, tetradecan-6-yl, 2-methylundecan-3-yl, 2-methyldecan-2-yl, 3-methylundecan-3-yl, 4-methyldodecan-4-yl or heptadeca-9-yl). In certain embodiments, R′ is

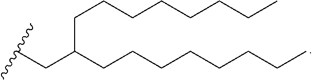 [SMILES: *CC(CCCCCCCC)CCCCCCCC]

In certain embodiments, R′ is unsubstituted C₁₋₁₈ alkyl. In certain embodiments, R′ is substituted C₁₋₁₈ alkyl (e.g., C₁₋₁₅ alkyl substituted with a C₃₋₆ carbocycle such as 1-cyclopropylnonyl).

In some embodiments, R″ is selected from the group consisting of C₃₋₁₄ alkyl and C₃₋₁₄ alkenyl. In some embodiments, R″ is C₃ alkyl, C₄ alkyl, C₅ alkyl, C₆ alkyl, C₇ alkyl, or C₈ alkyl. In some embodiments, R″ is C₉ alkyl, C₁₀ alkyl, C₁₁ alkyl, C₁₂ alkyl, C₁₃ alkyl, or C₁₄ alkyl.

In some embodiments, M′ is —C(O)O—. In some embodiments, M′ is —OC(O)—. In other embodiments, M′ is an aryl group or heteroaryl group. For example, M′ can be selected from the group consisting of phenyl, oxazole, and thiazole.

In some embodiments, M is —C(O)O—In some embodiments, M is —OC(O)—. In some embodiments, M is —C(O)N(R′)—. In some embodiments, M is —P(O)(OR′)O—.

In other embodiments, M is an aryl group or heteroaryl group. For example, M can be selected from the group consisting of phenyl, oxazole, and thiazole.

In some embodiments, M is the same as M′. In other embodiments, M is different from M′.

In some embodiments, each R₅ is H. In certain such embodiments, each R₆ is also H.

In some embodiments, R₇ is H. In other embodiments, R₇ is C₁₋₃ alkyl (e.g., methyl, ethyl, propyl, or i-propyl).

In some embodiments, R₂ and R₃ are independently C₅₋₁₄ alkyl or C₅₋₁₄ alkenyl.

In some embodiments, R₂ and R₃ are the same. In some embodiments, R₂ and R₃ are C₈ alkyl. In certain embodiments, R₂ and R₃ are C₂ alkyl. In other embodiments, R₂ and R₃ are C₃ alkyl. In some embodiments, R₂ and R₃ are C₄ alkyl. In certain embodiments, R₂ and R₃ are C₅ alkyl. In other embodiments, R₂ and R₃ are C₆ alkyl. In some embodiments, R₂ and R₃ are C₇ alkyl.

In other embodiments, R₂ and R₃ are different. In certain embodiments, R₂ is C₈ alkyl. In some embodiments, R₃ is C₁₋₇ (e.g., C₁, C₂, C₃, C₄, C₅, C₆, or C₇ alkyl) or C₉ alkyl.

In some embodiments, R₇ and R₃ are H.

In certain embodiments, R₂ is H.

In some embodiments, m is 5, 7, or 9.

In some embodiments, R₄ is selected from —(CH₂)ₙQ and —(CH₂)ₙCHQR.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), —C(R)N(R)₂C(O)OR, a carbocycle, and a heterocycle.

In certain embodiments, Q is —OH.

In certain embodiments, Q is a substituted or unsubstituted 5- to 10-membered heteroaryl, e.g., Q is an imidazole, a pyrimidine, a purine, 2-amino-1,9-dihydro-6H-purin-6-one-9-yl (or guanin-9-yl), adenin-9-yl, cytosin-1-yl, or uracil-1-yl. In certain embodiments, Q is a substituted 5- to 14-membered heterocycloalkyl, e.g., substituted with one or more substituents selected from oxo (═O), OH, amino, and C₁₋₃ alkyl. For example, Q is 4-methylpiperazinyl, 4-(4-methoxybenzyl) piperazinyl, or isoindolin-2-yl-1,3-dione.

In certain embodiments, Q is an unsubstituted or substituted C₆₋₁₀ aryl (such as phenyl) or C₃₋₆ cycloalkyl.

In some embodiments, n is 1. In other embodiments, n is 2. In further embodiments, n is 3. In certain other embodiments, n is 4. For example, R₄ can be (CH₂)₂OH. For example, R₄ can be —(CH₂)₃OH. For example, R₄ can be —(CH₂)₄OH. For example, R₄ can be benzyl. For example, R₄ can be 4-methoxybenzyl.

In some embodiments, R₄ is a C₃₋₆ carbocycle. In some embodiments, R₄ is a C₃₋₆ cycloalkyl. For example, R₄ can be cyclohexyl optionally substituted with e.g., OH, halo, C₁₋₆ alkyl, etc. For example, R₄ can be 2-hydroxycyclohexyl.

In some embodiments, R is H.

In some embodiments, R is unsubstituted C₁₋₃ alkyl or unsubstituted C₂₋₃ alkenyl. For example, R₄ can be —CH₂CH(OH)CH₃ or —CH₂CH(OH)CH₂CH₃.

In some embodiments, R is substituted C₁₋₃ alkyl, e.g., CH₂OH. For example, R₄ can be —CH₂CH(OH)CH₂OH.

In some embodiments, R₂ and R₃, together with the atom to which they are attached, form a heterocycle or carbocycle. In some embodiments, R₂ and R₃, together with the atom to which they are attached, form a 5- to 14-membered aromatic or non-aromatic heterocycle having one or more heteroatoms selected from N, O, S, and P. In some embodiments, R₂ and R₃, together with the atom to which they are attached, form an optionally substituted C₃₋₂₀ carbocycle (e.g., C₃₋₁₈ carbocycle, C₃₋₁₅ carbocycle, C₃₋₁₂ carbocycle, or C₃₋₁₀ carbocycle), either aromatic or non-aromatic. In some embodiments, R₂ and R₃, together with the atom to which they are attached, form a C₃₋₆ carbocycle. In other embodiments, R₂ and R₃, together with the atom to which they are attached, form a C₆ carbocycle, such as a cyclohexyl or phenyl group. In certain embodiments, the heterocycle or C₃₋₆ carbocycle is substituted with one or more alkyl groups (e.g., at the same ring atom or at adjacent or non-adjacent ring atoms). For example, R₂ and R₃, together with the atom to which they are attached, can form a cyclohexyl or phenyl group bearing one or more C₅ alkyl substitutions. In certain embodiments, the heterocycle or C₃₋₆ carbocycle formed by R₂ and R₃, is substituted with a carbocycle groups. For example, R₂ and R₃, together with the atom to which they are attached, can form a cyclohexyl or phenyl group that is substituted with cyclohexyl. In some embodiments, R₂ and R₃, together with the atom to which they are attached, form a C₇₋₁₅ carbocycle, such as a cycloheptyl, cyclopentadecanyl, or naphthyl group.

In some embodiments, R₄ is selected from —(CH₂)ₙQ and —(CH₂)ₙCHQR. In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle. In other embodiments, Q is selected from the group consisting of an imidazole, a pyrimidine, and a purine.

In some embodiments, R₂ and R₃, together with the atom to which they are attached, form a heterocycle or carbocycle. In some embodiments, R₂ and R₃, together with the atom to which they are attached, form a C₃₋₆ carbocycle, such as a phenyl group. In certain embodiments, the heterocycle or C₃₋₆ carbocycle is substituted with one or more alkyl groups (e.g., at the same ring atom or at adjacent or non-adjacent ring atoms). For example, R₂ and R₃, together with the atom to which they are attached, can form a phenyl group bearing one or more C₅ alkyl substitutions.

In some embodiments, the pharmaceutical compositions of the present disclosure, the compound of formula (I) is selected from the group consisting of:

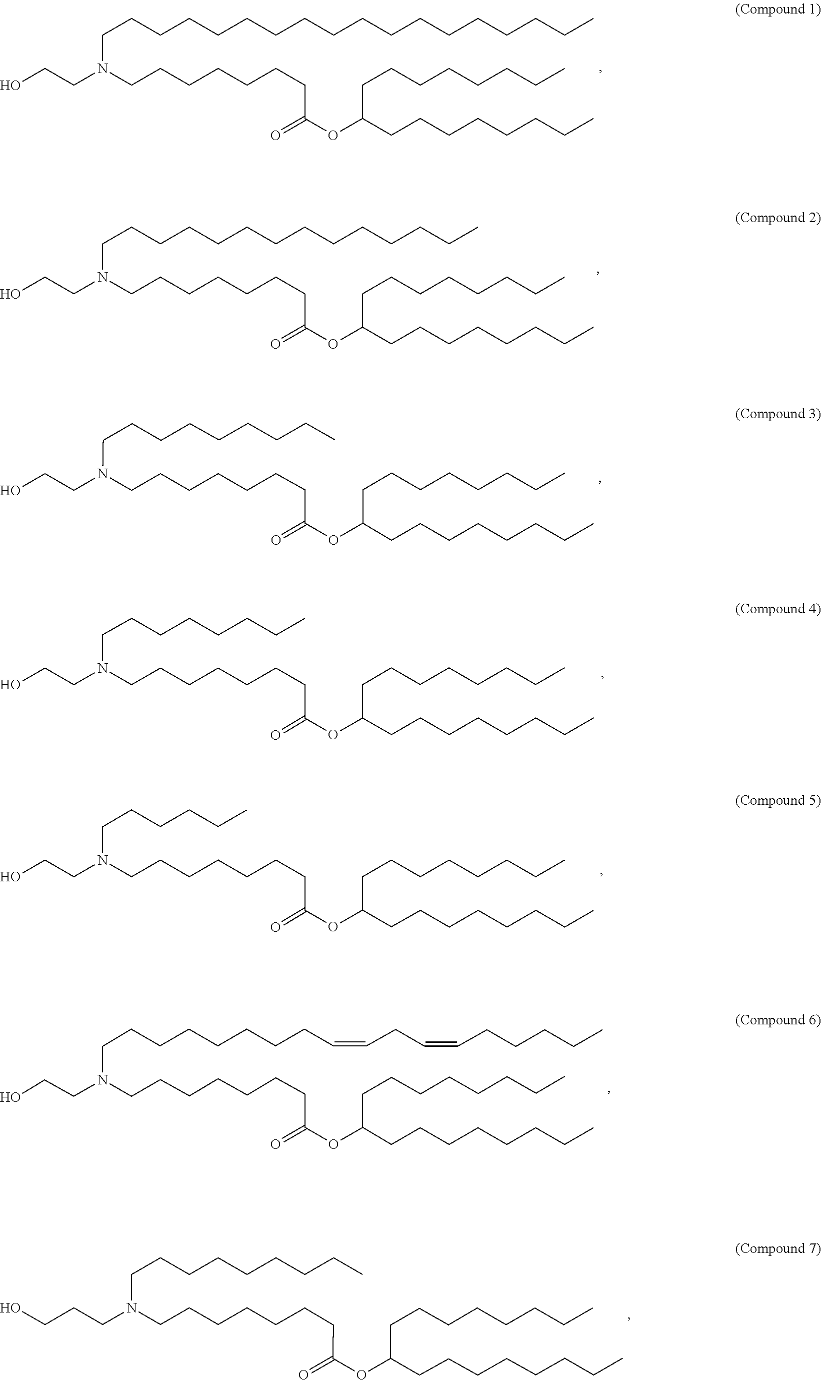

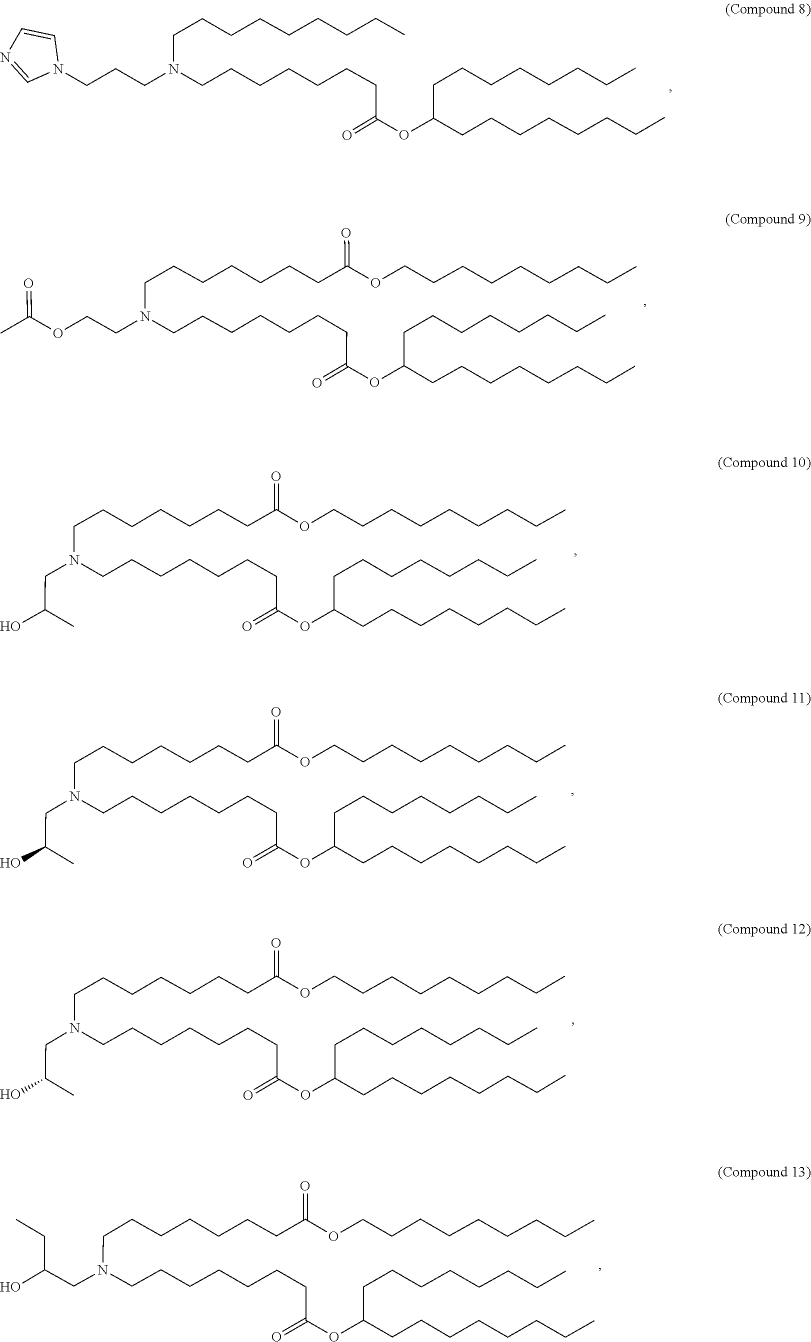

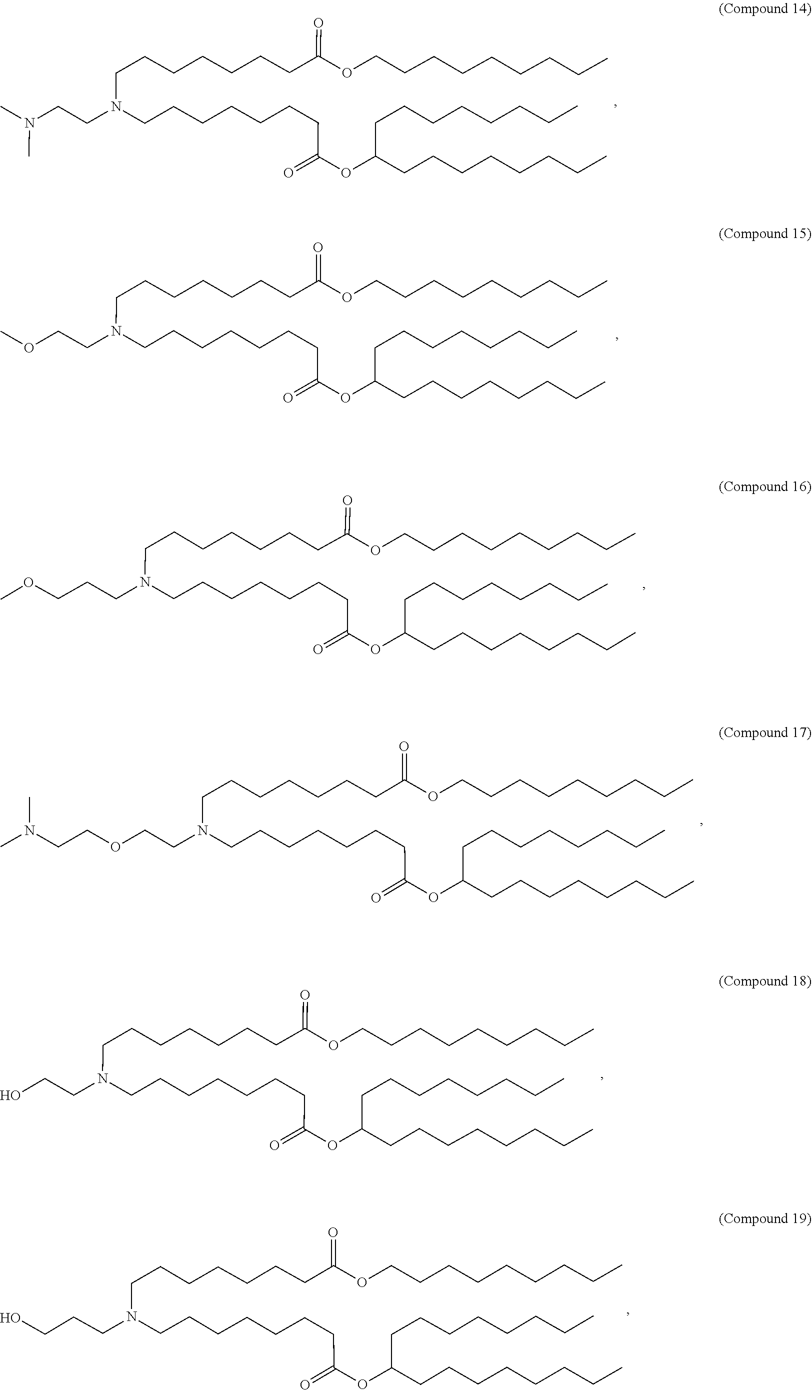

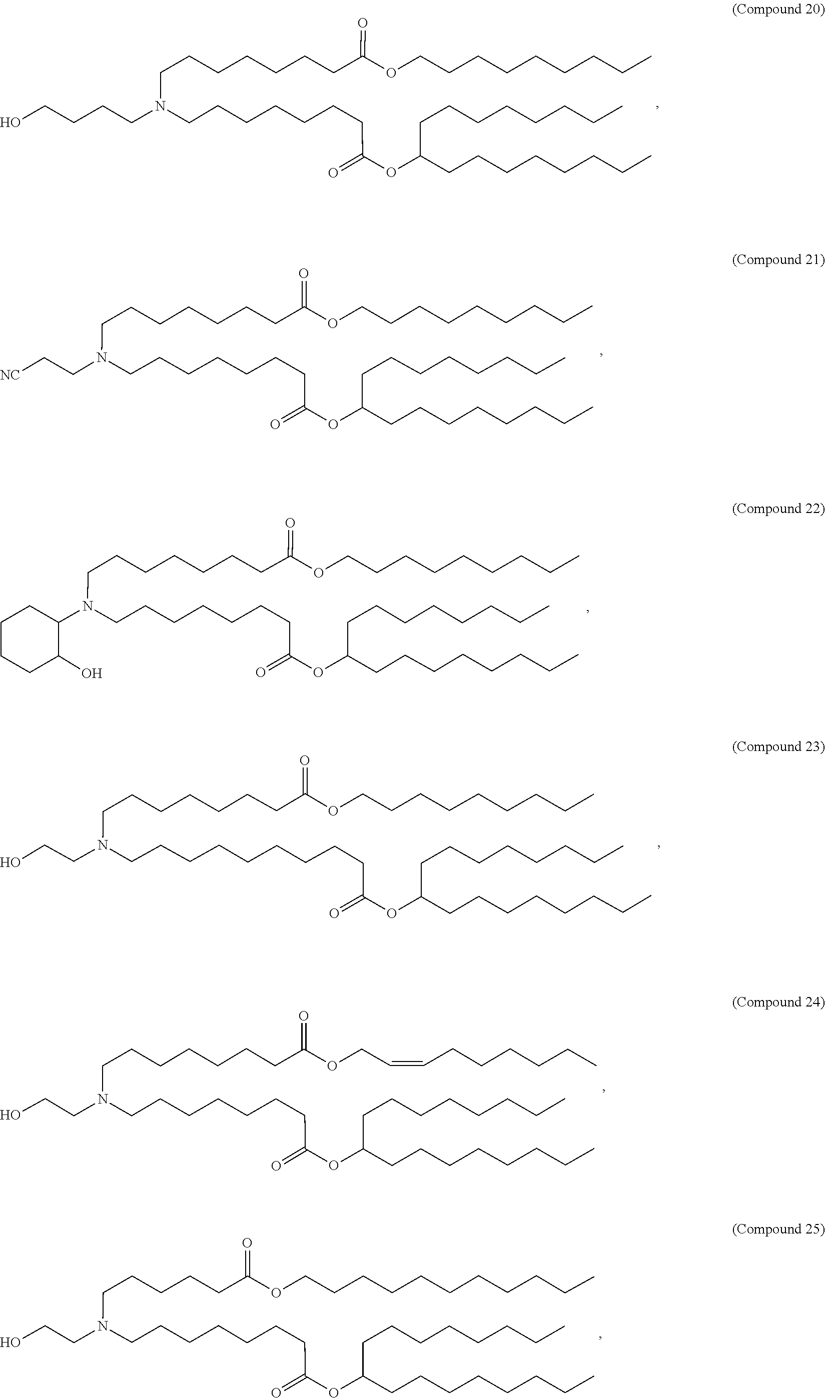 [SMILES: CCCCCCCCCOC(=O)CCCCCCCN(CCCCO)CCCCCCCC(=O)OC(CCCCCCCC)CCCCCCCC ; CCCCCCCCCOC(=O)CCCCCCCN(CCC#N)CCCCCCCC(=O)OC(CCCCCCCC)CCCCCCCC ; CCCCCCCCCOC(=O)CCCCCCCN(CCCCCCCC(=O)OC(CCCCCCCC)CCCCCCCC)C1CCCCC1O ; CCCCCCCCCOC(=O)CCCCCCCN(CCO)CCCCCCCCCC(=O)OC(CCCCCCCC)CCCCCCCC ; CCCCCC/C=C\COC(=O)CCCCCCCN(CCO)CCCCCCCC(=O)OC(CCCCCCCC)CCCCCCCC ; CCCCCCCCCCCOC(=O)CCCCCN(CCO)CCCCCCCC(=O)OC(CCCCCCCC)CCCCCCCC]

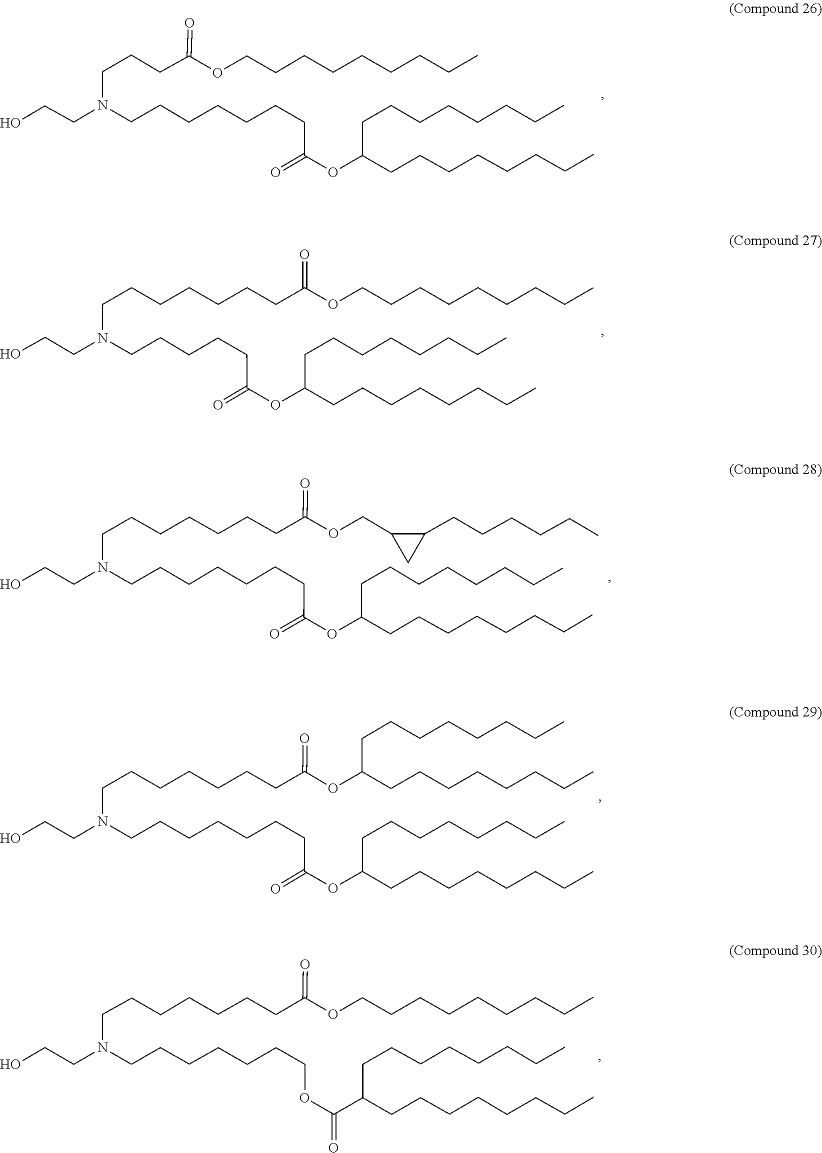 [SMILES: CCCCCCCCCOC(=O)CCCN(CCO)CCCCCCCC(=O)OC(CCCCCCCC)CCCCCCCC ; CCCCCCCCCOC(=O)CCCCCCCN(CCO)CCCCCC(=O)OC(CCCCCCCC)CCCCCCCC ; CCCCCCCCC(CCCCCCCC)OC(=O)CCCCCCCN(CCO)CCCCCCCC(=O)OCC1CC1CCCCCC ; CCCCCCCCC(CCCCCCCC)OC(=O)CCCCCCCN(CCO)CCCCCCCC(=O)OC(CCCCCCCC)CCCCCCCC ; CCCCCCCCCOC(=O)CCCCCCCN(CCO)CCCCCCCOC(=O)C(CCCCCCCC)CCCCCCCC]

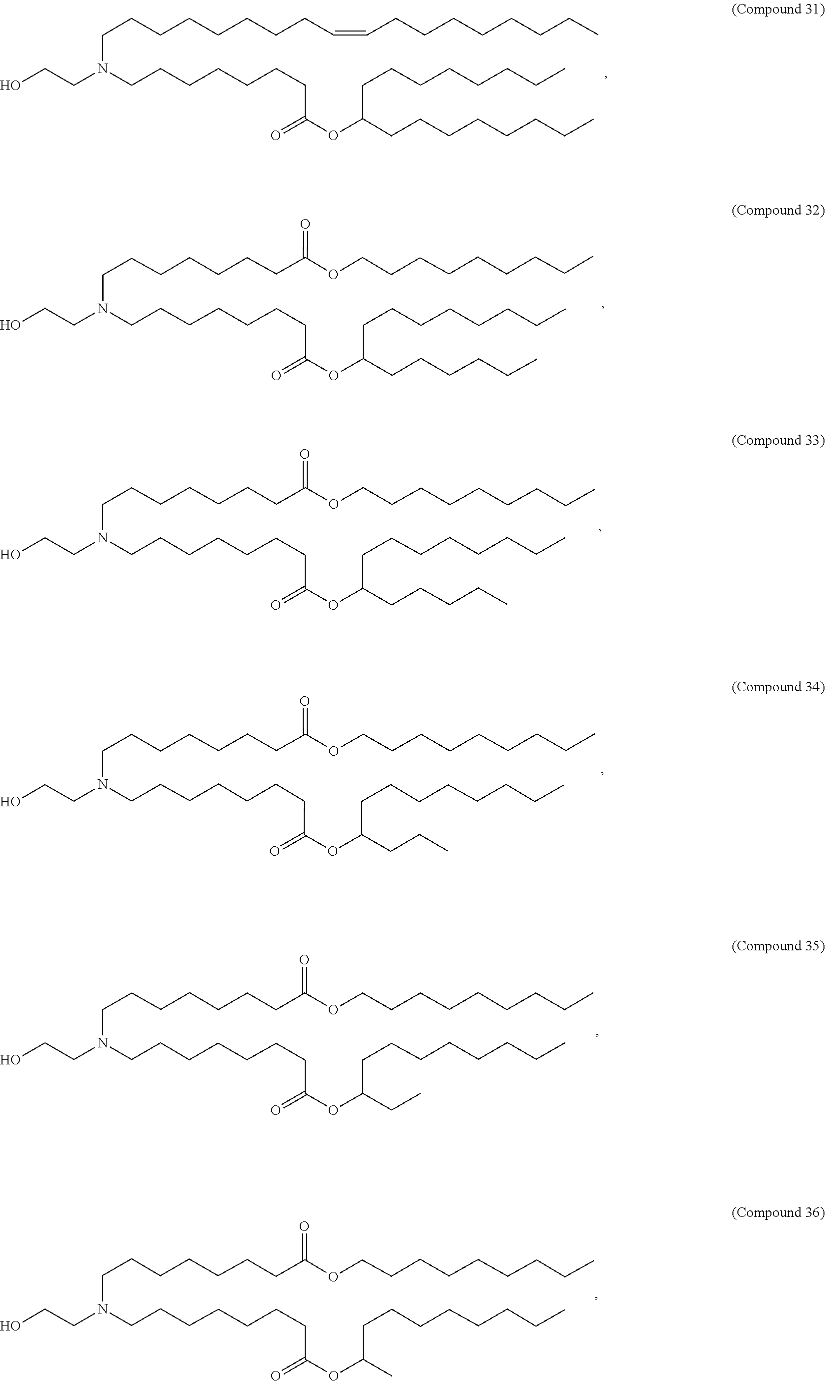 [SMILES: CCCCCCCC/C=C\CCCCCCCCN(CCO)CCCCCCCC(=O)OC(CCCCCCCC)CCCCCCCC ; CCCCCCCCCOC(=O)CCCCCCCN(CCO)CCCCCCCC(=O)OC(CCCCCC)CCCCCCCC ; CCCCCCCCCOC(=O)CCCCCCCN(CCO)CCCCCCCC(=O)OC(CCCCC)CCCCCCCC ; CCCCCCCCCOC(=O)CCCCCCCN(CCO)CCCCCCCC(=O)OC(CCC)CCCCCCCC ; CCCCCCCCCOC(=O)CCCCCCCN(CCO)CCCCCCCC(=O)OC(CC)CCCCCCCC ; CCCCCCCCCOC(=O)CCCCCCCN(CCO)CCCCCCCC(=O)OC(C)CCCCCCCC]

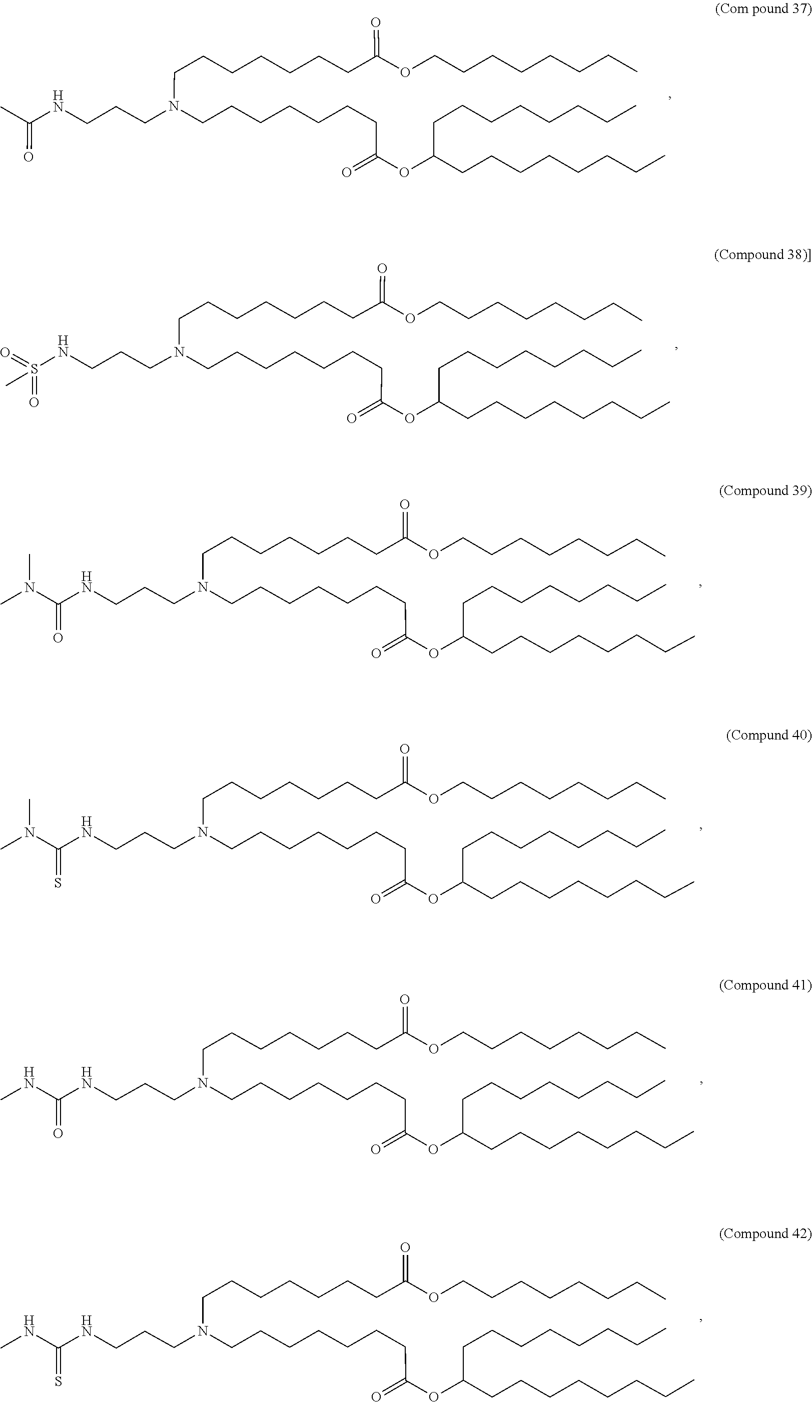

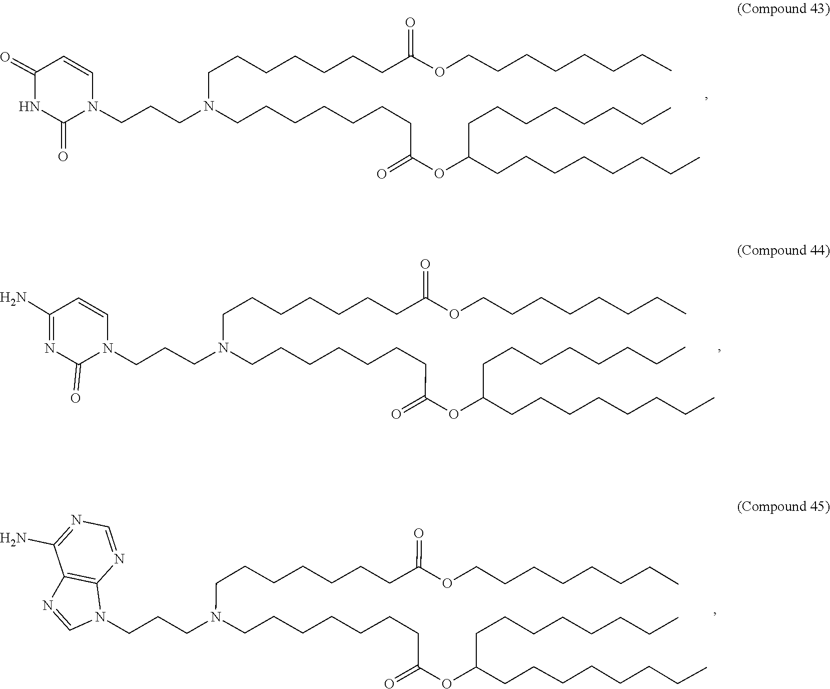 [SMILES: CCCCCCCCOC(=O)CCCCCCCN(CCCCCCCC(=O)OC(CCCCCCCC)CCCCCCCC)CCCn1ccc(=O)[nH]c1=O ; CCCCCCCCOC(=O)CCCCCCCN(CCCCCCCC(=O)OC(CCCCCCCC)CCCCCCCC)CCCn1ccc(N)nc1=O ; CCCCCCCCOC(=O)CCCCCCCN(CCCCCCCC(=O)OC(CCCCCCCC)CCCCCCCC)CCCn1cnc2c(N)ncnc21]

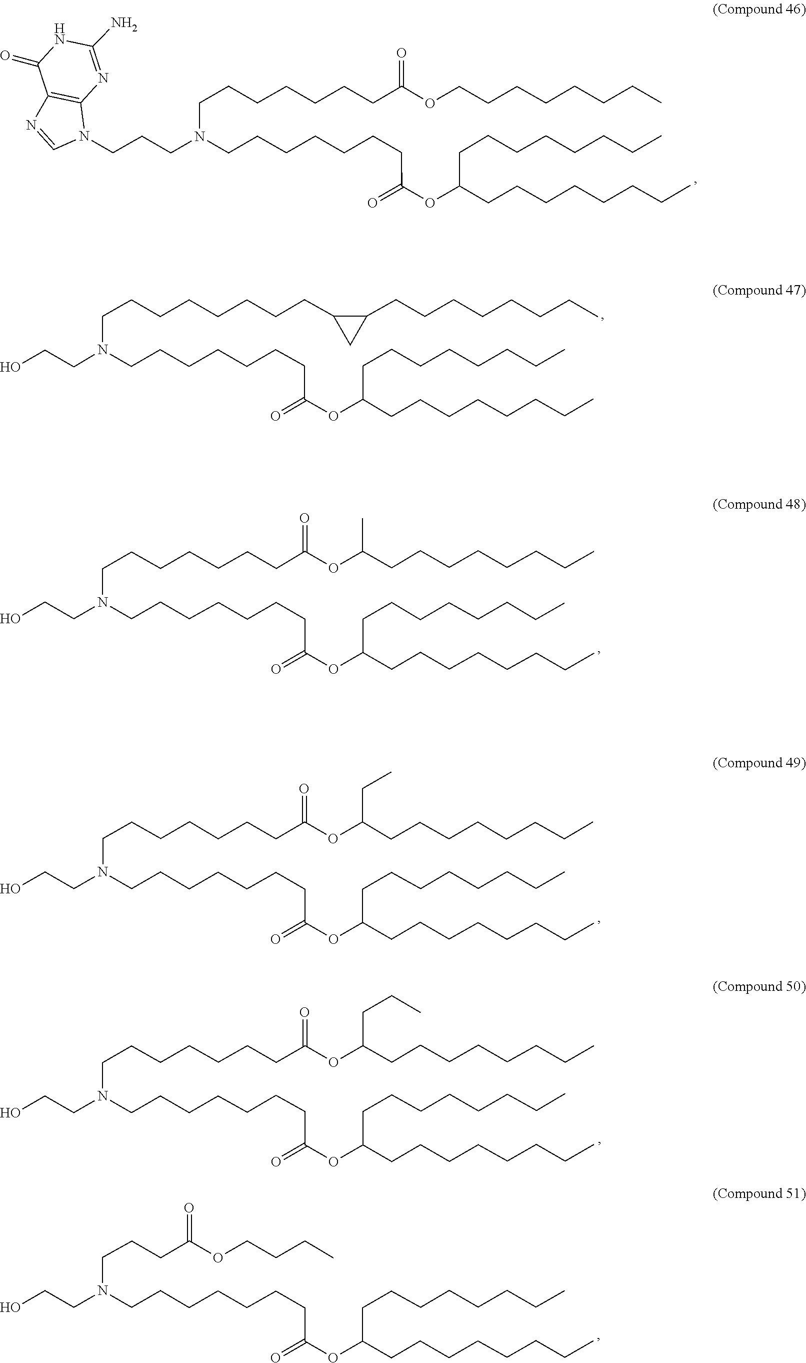 [SMILES: CCCCCCCCOC(=O)CCCCCCCN(CCCCCCCC(=O)OC(CCCCCCCC)CCCCCCCC)CCCn1cnc2c(=O)[nH]c(N)nc21 ; CCCCCCCCC(CCCCCCCC)OC(=O)CCCCCCCN(CCO)CCCCCCCCC1CC1CCCCCCCC ; CCCCCCCCC(C)OC(=O)CCCCCCCN(CCO)CCCCCCCC(=O)OC(CCCCCCCC)CCCCCCCC ; CCCCCCCCC(CC)OC(=O)CCCCCCCN(CCO)CCCCCCCC(=O)OC(CCCCCCCC)CCCCCCCC ; CCCCCCCCC(CCC)OC(=O)CCCCCCCN(CCO)CCCCCCCC(=O)OC(CCCCCCCC)CCCCCCCC ; CCCCCCCCC(CCCCCCCC)OC(=O)CCCCCCCN(CCO)CCCC(=O)OCCCC]

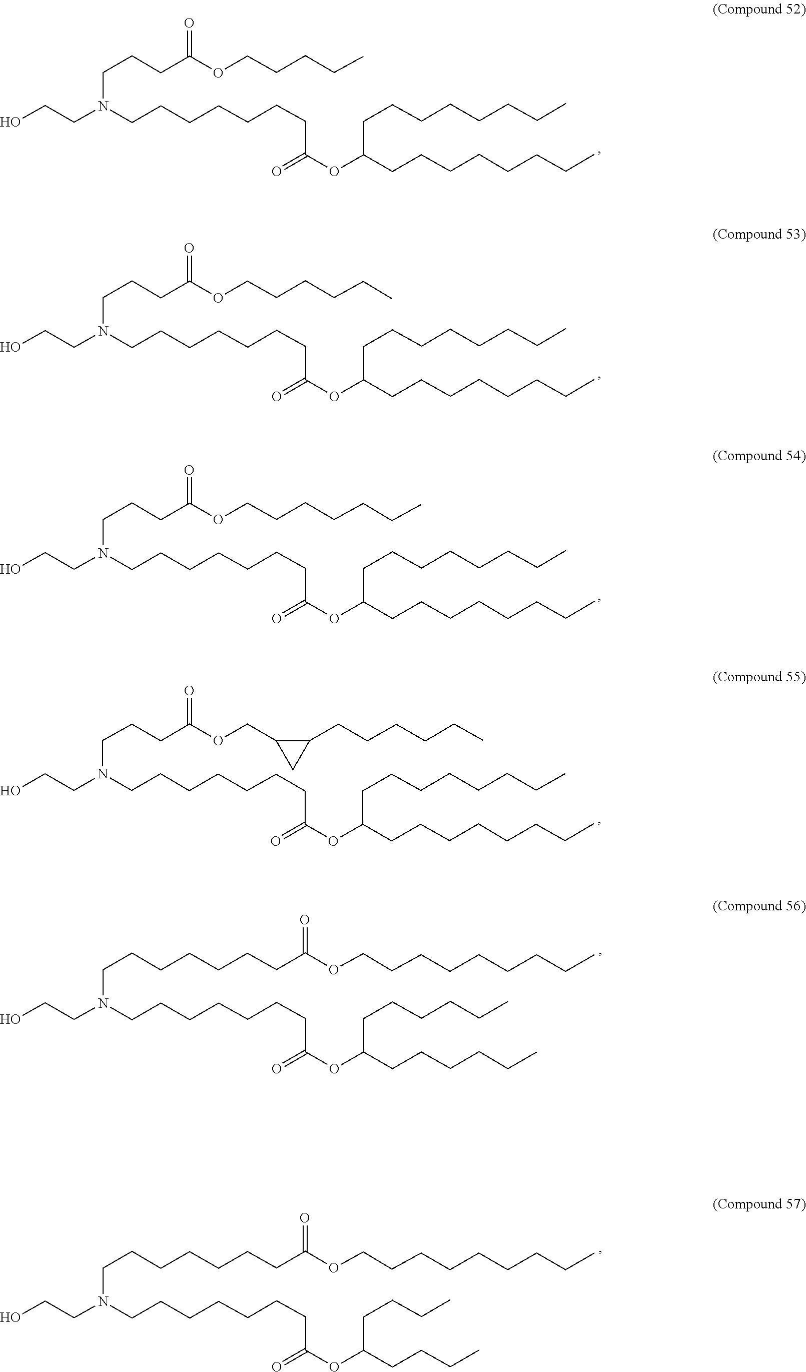 [SMILES: CCCCCCCCC(CCCCCCCC)OC(=O)CCCCCCCN(CCO)CCCC(=O)OCCCCC ; CCCCCCCCC(CCCCCCCC)OC(=O)CCCCCCCN(CCO)CCCC(=O)OCCCCCC ; CCCCCCCCC(CCCCCCCC)OC(=O)CCCCCCCN(CCO)CCCC(=O)OCCCCCCC ; CCCCCCCCC(CCCCCCCC)OC(=O)CCCCCCCN(CCO)CCCC(=O)OCC1CC1CCCCCC ; CCCCCCCCCOC(=O)CCCCCCCN(CCO)CCCCCCCC(=O)OC(CCCCCC)CCCCCC ; CCCCCCCCCOC(=O)CCCCCCCN(CCO)CCCCCCCC(=O)OC(CCCC)CCCC]

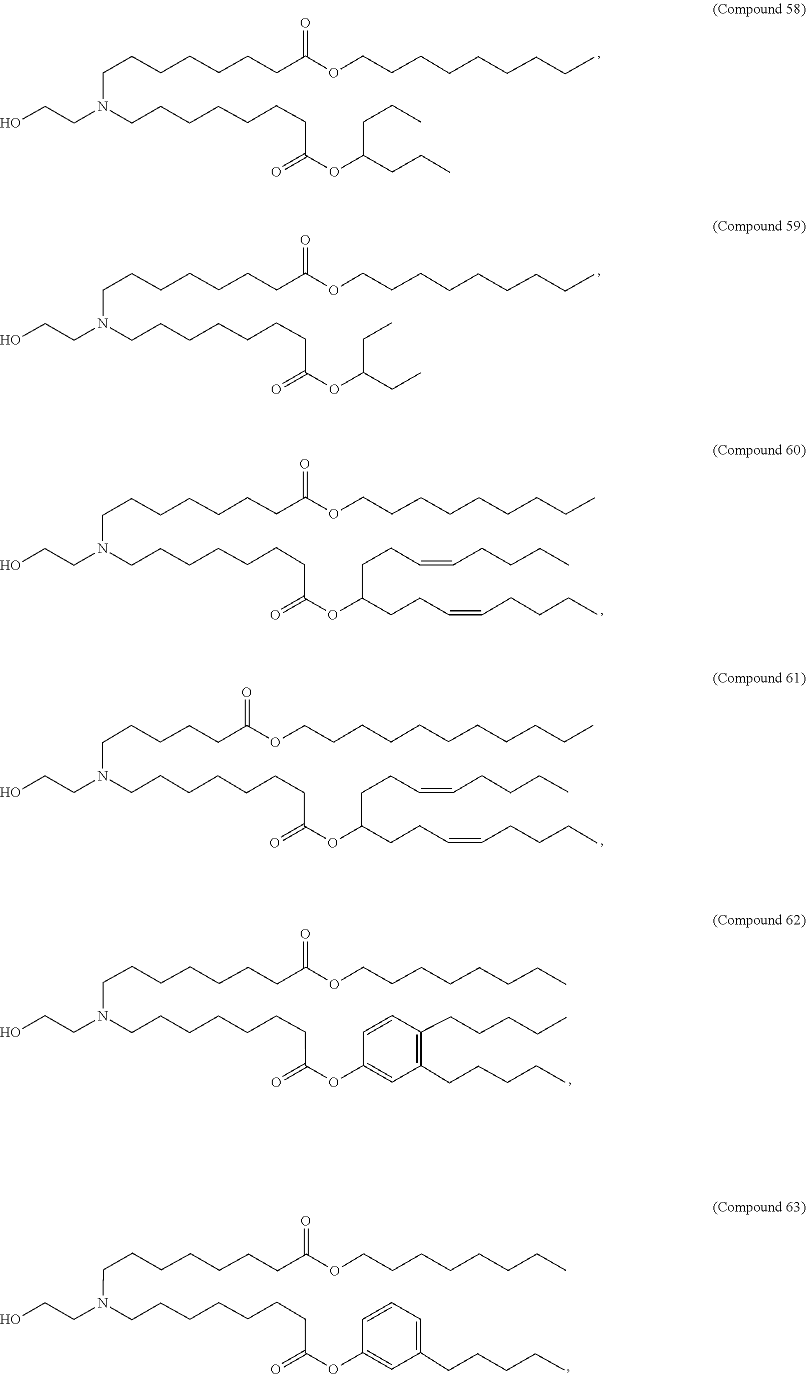 [SMILES: CCCCCCCCCOC(=O)CCCCCCCN(CCO)CCCCCCCC(=O)OC(CCC)CCC ; CCCCCCCCCOC(=O)CCCCCCCN(CCO)CCCCCCCC(=O)OC(CC)CC ; CCCC/C=C\CCC(CC/C=C\CCCC)OC(=O)CCCCCCCN(CCO)CCCCCCCC(=O)OCCCCCCCCC ; CCCC/C=C\CCC(CC/C=C\CCCC)OC(=O)CCCCCCCN(CCO)CCCCCC(=O)OCCCCCCCCCCC ; CCCCCCCCOC(=O)CCCCCCCN(CCO)CCCCCCCC(=O)Oc1ccc(CCCCC)c(CCCCC)c1 ; CCCCCCCCOC(=O)CCCCCCCN(CCO)CCCCCCCC(=O)Oc1cccc(CCCCC)c1]

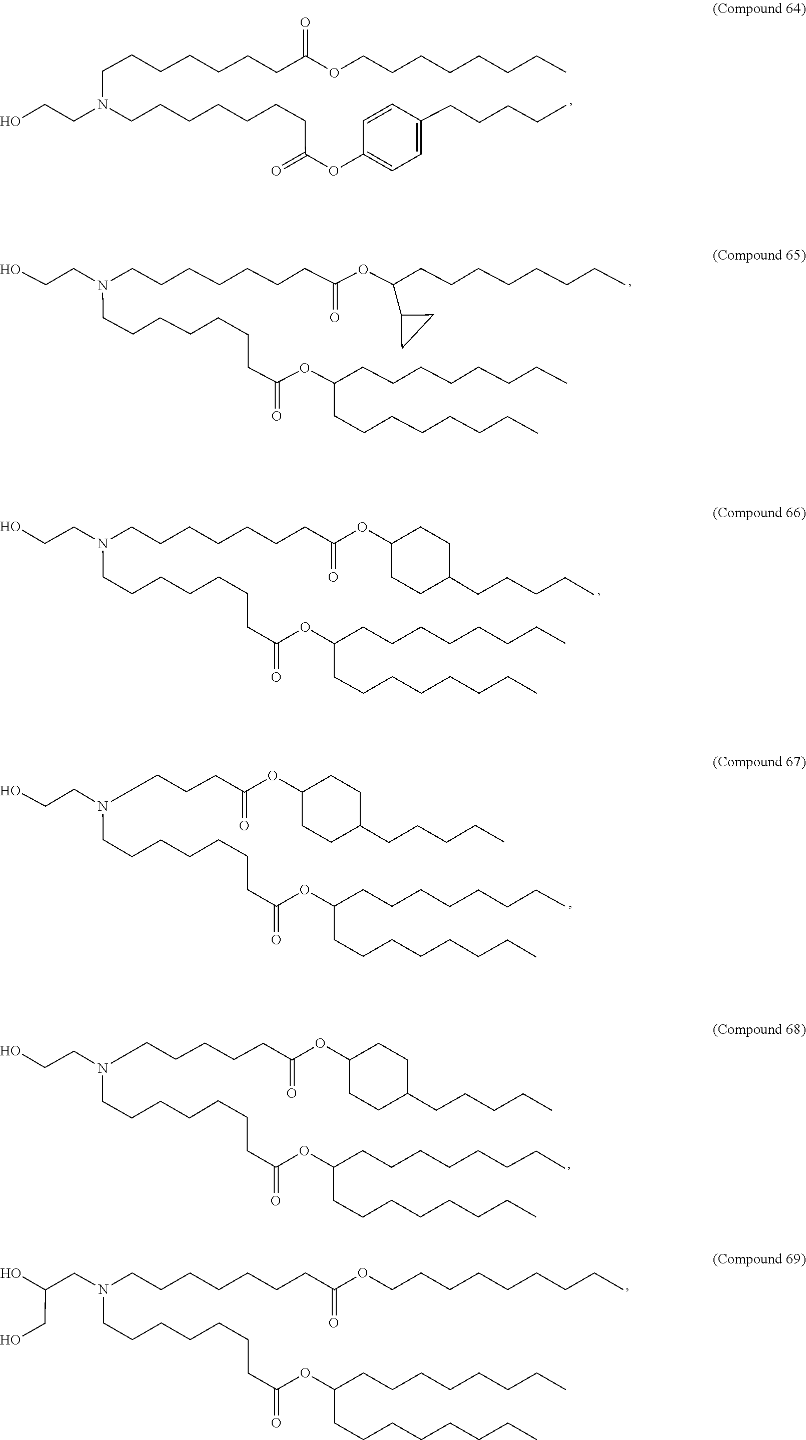 [SMILES: CCCCCCCCOC(=O)CCCCCCCN(CCO)CCCCCCCC(=O)Oc1ccc(CCCCC)cc1 ; CCCCCCCCC(CCCCCCCC)OC(=O)CCCCCCCN(CCO)CCCCCCCC(=O)OC(CCCCCCCC)C1CC1 ; CCCCCCCCC(CCCCCCCC)OC(=O)CCCCCCCN(CCO)CCCCCCCC(=O)OC1CCC(CCCCC)CC1 ; CCCCCCCCC(CCCCCCCC)OC(=O)CCCCCCCN(CCO)CCCC(=O)OC1CCC(CCCCC)CC1 ; CCCCCCCCC(CCCCCCCC)OC(=O)CCCCCCCN(CCO)CCCCCC(=O)OC1CCC(CCCCC)CC1 ; CCCCCCCCCOC(=O)CCCCCCCN(CCCCCCCC(=O)OC(CCCCCCCC)CCCCCCCC)CC(O)CO]

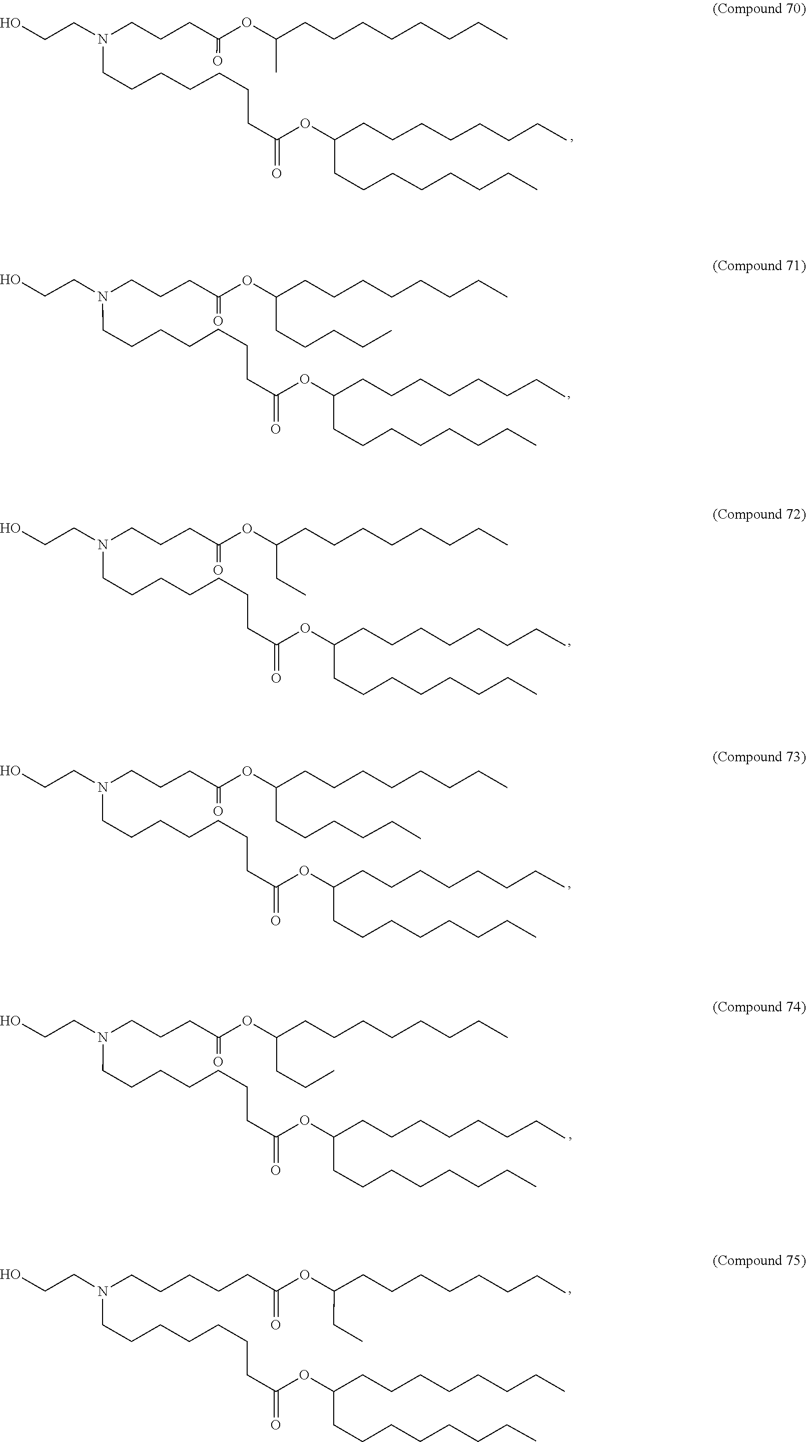 [SMILES: CCCCCCCCC(C)OC(=O)CCCN(CCO)CCCCCCCC(=O)OC(CCCCCCCC)CCCCCCCC ; CCCCCCCCC(CCCCCCCC)OC(=O)CCCCCCCN(CCO)CCCC(=O)OC(CCCCC)CCCCCCCC ; CCCCCCCCC(CC)OC(=O)CCCN(CCO)CCCCCCCC(=O)OC(CCCCCCCC)CCCCCCCC ; CCCCCCCCC(CCCCCCCC)OC(=O)CCCCCCCN(CCO)CCCC(=O)OC(CCCCCC)CCCCCCCC ; CCCCCCCCC(CCC)OC(=O)CCCN(CCO)CCCCCCCC(=O)OC(CCCCCCCC)CCCCCCCC ; CCCCCCCCC(CC)OC(=O)CCCCCN(CCO)CCCCCCCC(=O)OC(CCCCCCCC)CCCCCCCC]

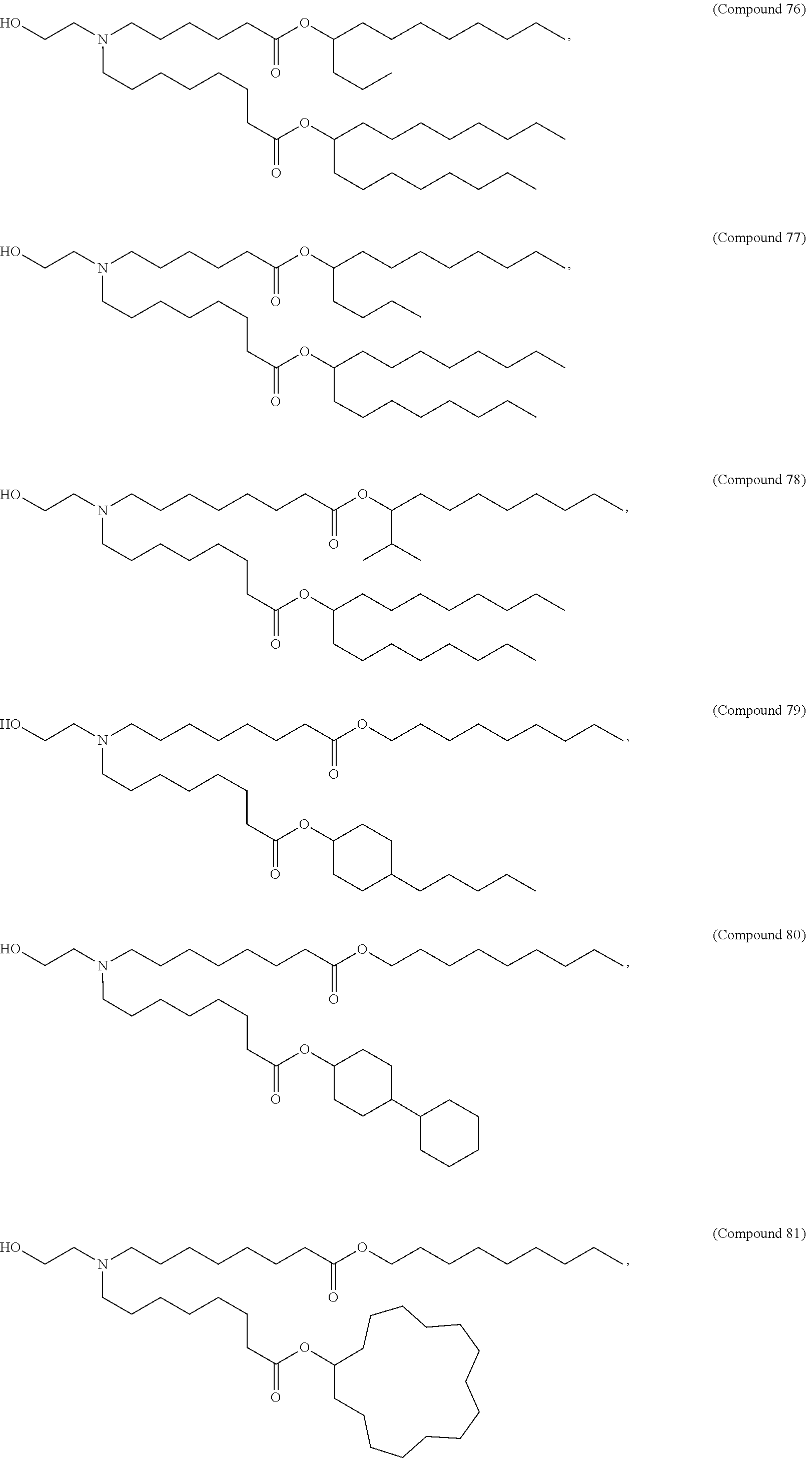 [SMILES: CCCCCCCCC(CCC)OC(=O)CCCCCN(CCO)CCCCCCCC(=O)OC(CCCCCCCC)CCCCCCCC ; CCCCCCCCC(CCCC)OC(=O)CCCCCN(CCO)CCCCCCCC(=O)OC(CCCCCCCC)CCCCCCCC ; CCCCCCCCC(CCCCCCCC)OC(=O)CCCCCCCN(CCO)CCCCCCCC(=O)OC(CCCCCCCC)C(C)C ; CCCCCCCCCOC(=O)CCCCCCCN(CCO)CCCCCCCC(=O)OC1CCC(CCCCC)CC1 ; CCCCCCCCCOC(=O)CCCCCCCN(CCO)CCCCCCCC(=O)OC1CCC(C2CCCCC2)CC1 ; CCCCCCCCCOC(=O)CCCCCCCN(CCO)CCCCCCCC(=O)OC1CCCCCCCCCCCCCC1]

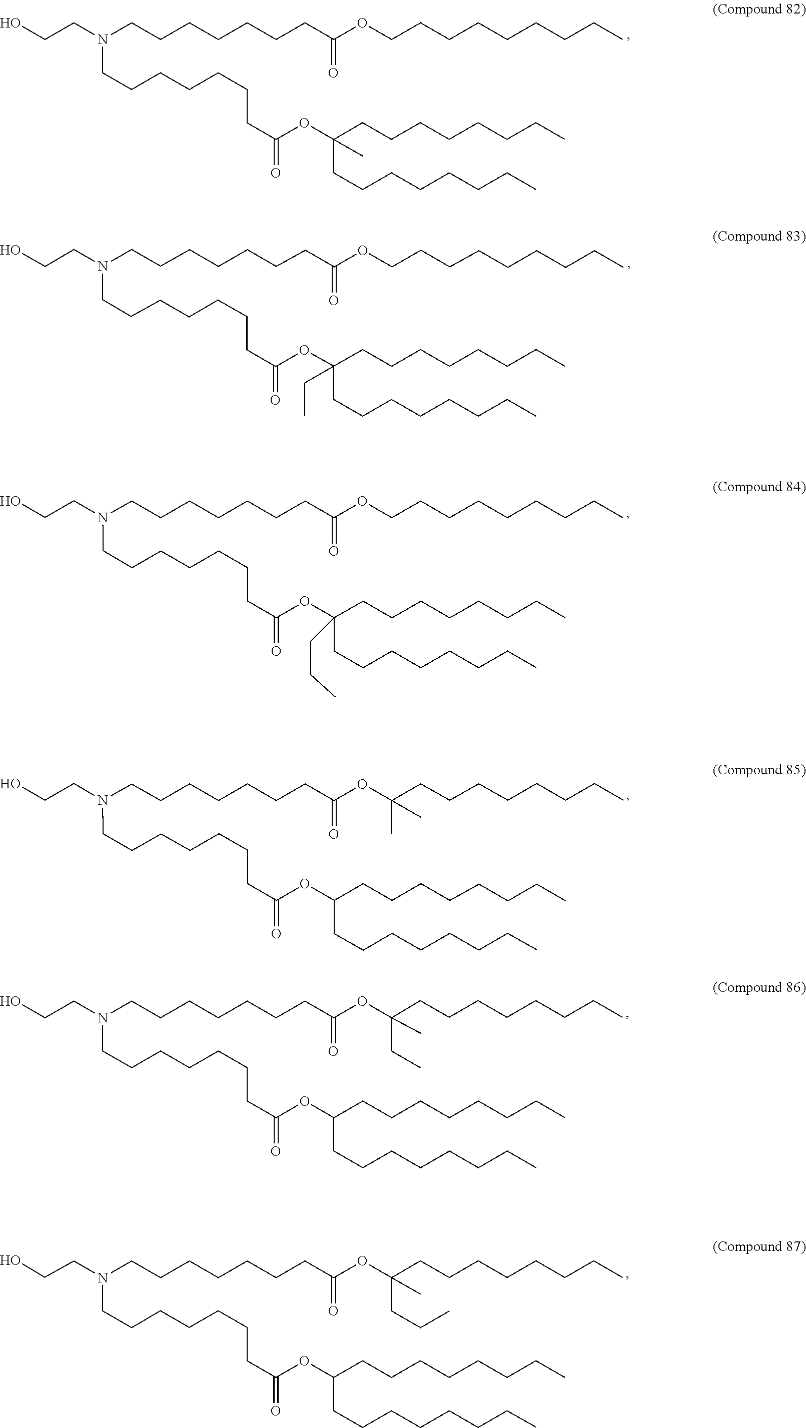 [SMILES: CCCCCCCCCOC(=O)CCCCCCCN(CCO)CCCCCCCC(=O)OC(C)(CCCCCCCC)CCCCCCCC ; CCCCCCCCCOC(=O)CCCCCCCN(CCO)CCCCCCCC(=O)OC(CC)(CCCCCCCC)CCCCCCCC ; CCCCCCCCCOC(=O)CCCCCCCN(CCO)CCCCCCCC(=O)OC(CCC)(CCCCCCCC)CCCCCCCC ; CCCCCCCCC(CCCCCCCC)OC(=O)CCCCCCCN(CCO)CCCCCCCC(=O)OC(C)(C)CCCCCCCC ; CCCCCCCCC(CCCCCCCC)OC(=O)CCCCCCCN(CCO)CCCCCCCC(=O)OC(C)(CC)CCCCCCCC ; CCCCCCCCC(CCCCCCCC)OC(=O)CCCCCCCN(CCO)CCCCCCCC(=O)OC(C)(CCC)CCCCCCCC]

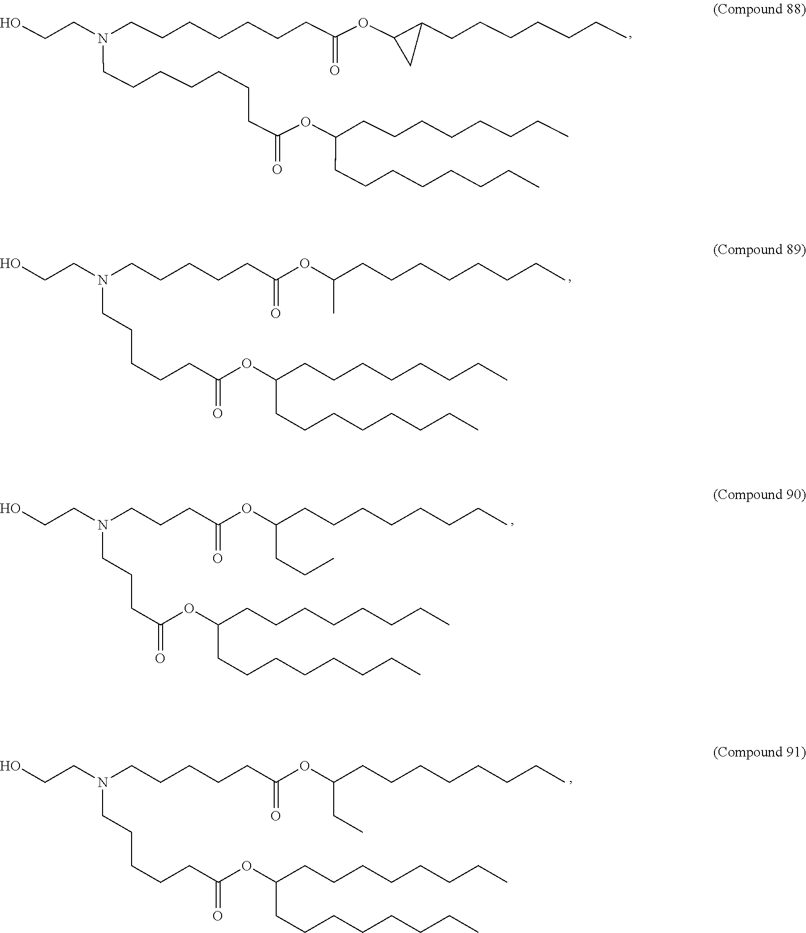 [SMILES: CCCCCCCCC(CCCCCCCC)OC(=O)CCCCCCCN(CCO)CCCCCCCC(=O)OC1CC1CCCCCCC ; CCCCCCCCC(C)OC(=O)CCCCCN(CCO)CCCCCC(=O)OC(CCCCCCCC)CCCCCCCC ; CCCCCCCCC(CCC)OC(=O)CCCN(CCO)CCCC(=O)OC(CCCCCCCC)CCCCCCCC ; CCCCCCCCC(CC)OC(=O)CCCCCN(CCO)CCCCCC(=O)OC(CCCCCCCC)CCCCCCCC]

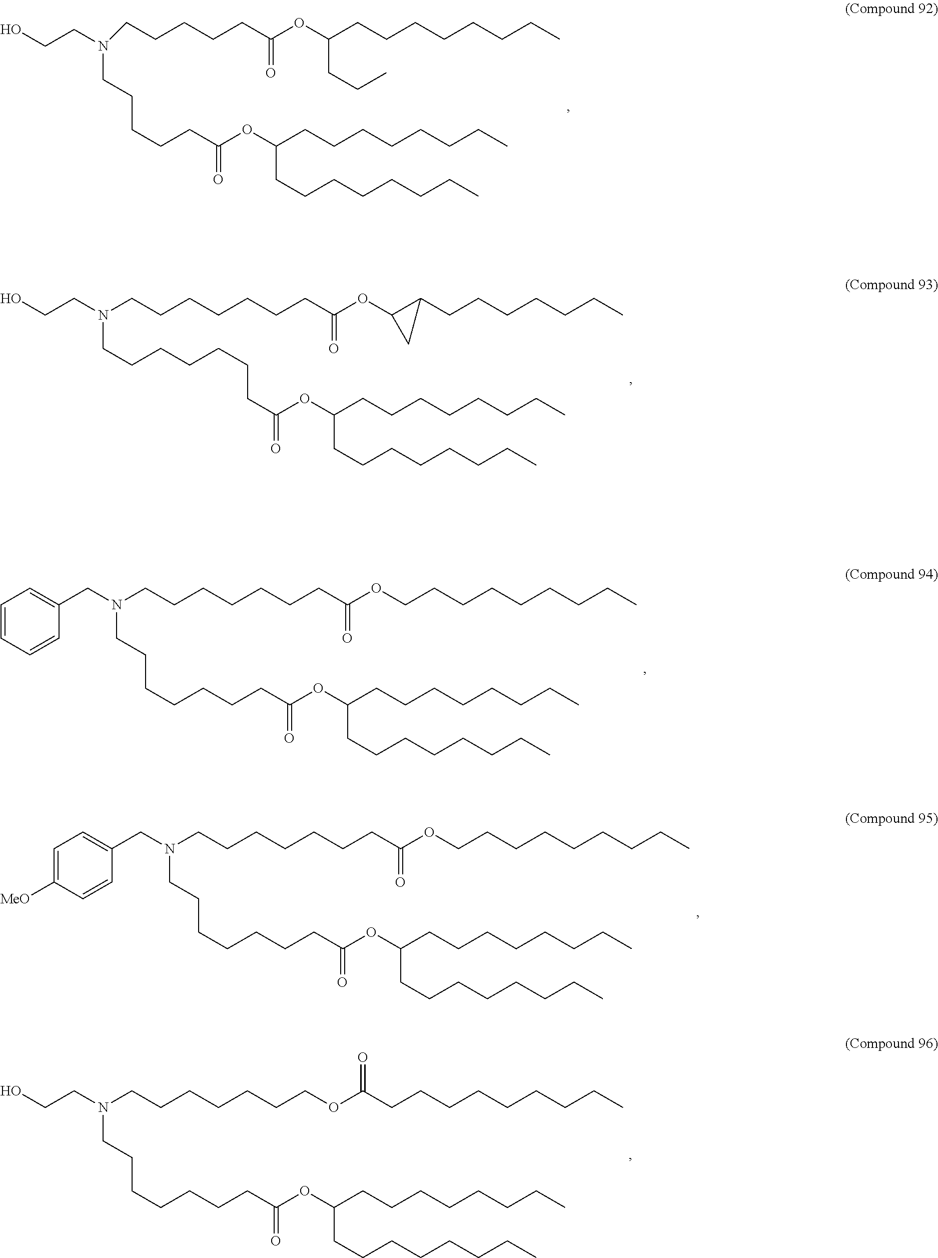 [SMILES: CCCCCCCCC(CCC)OC(=O)CCCCCN(CCO)CCCCCC(=O)OC(CCCCCCCC)CCCCCCCC ; CCCCCCCCC(CCCCCCCC)OC(=O)CCCCCCCN(CCO)CCCCCCCC(=O)OC1CC1CCCCCCC ; CCCCCCCCCOC(=O)CCCCCCCN(CCCCCCCC(=O)OC(CCCCCCCC)CCCCCCCC)Cc1ccccc1 ; CCCCCCCCCOC(=O)CCCCCCCN(CCCCCCCC(=O)OC(CCCCCCCC)CCCCCCCC)Cc1ccc(OC)cc1 ; CCCCCCCCCC(=O)OCCCCCCCN(CCO)CCCCCCCC(=O)OC(CCCCCCCC)CCCCCCCC]

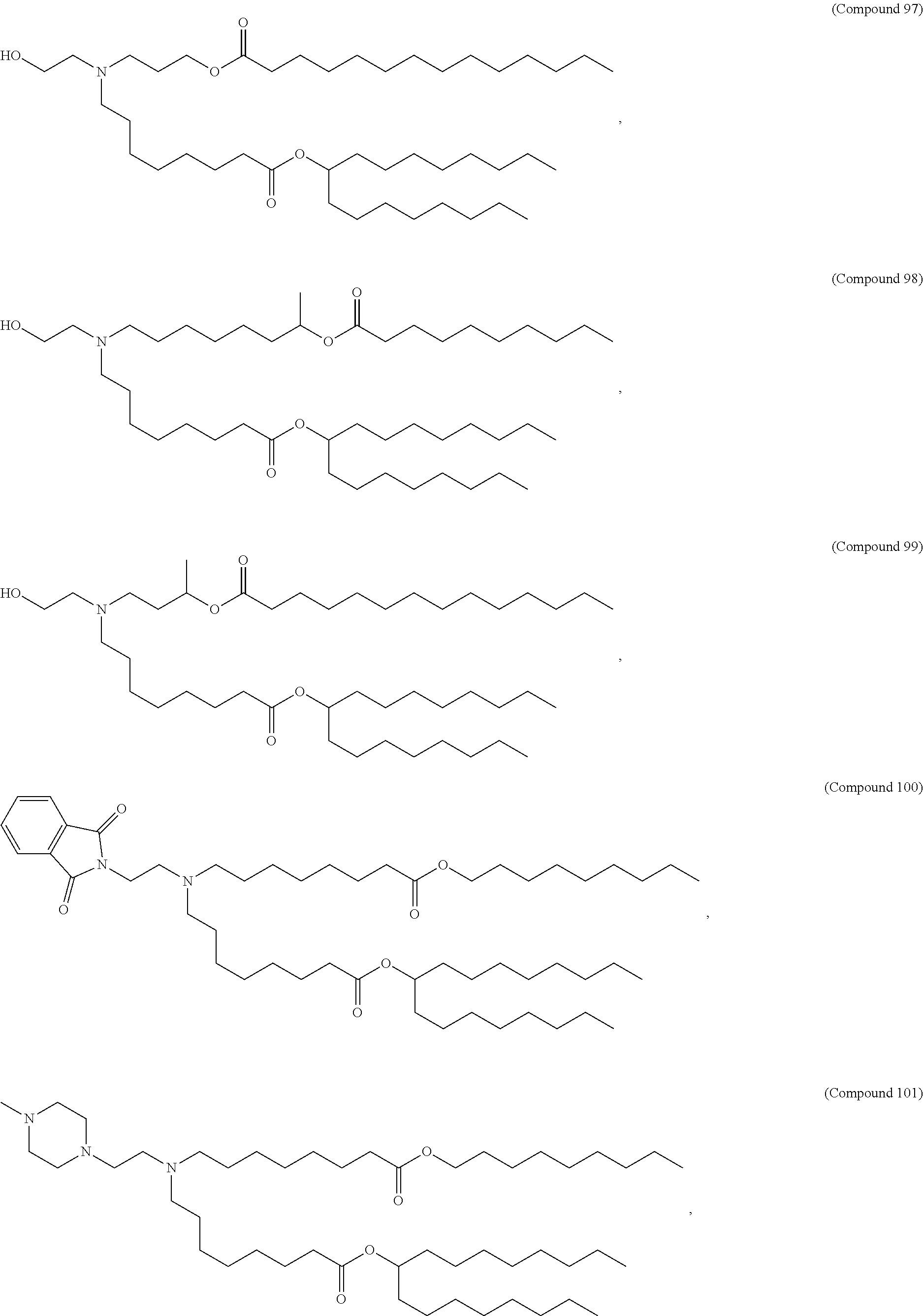

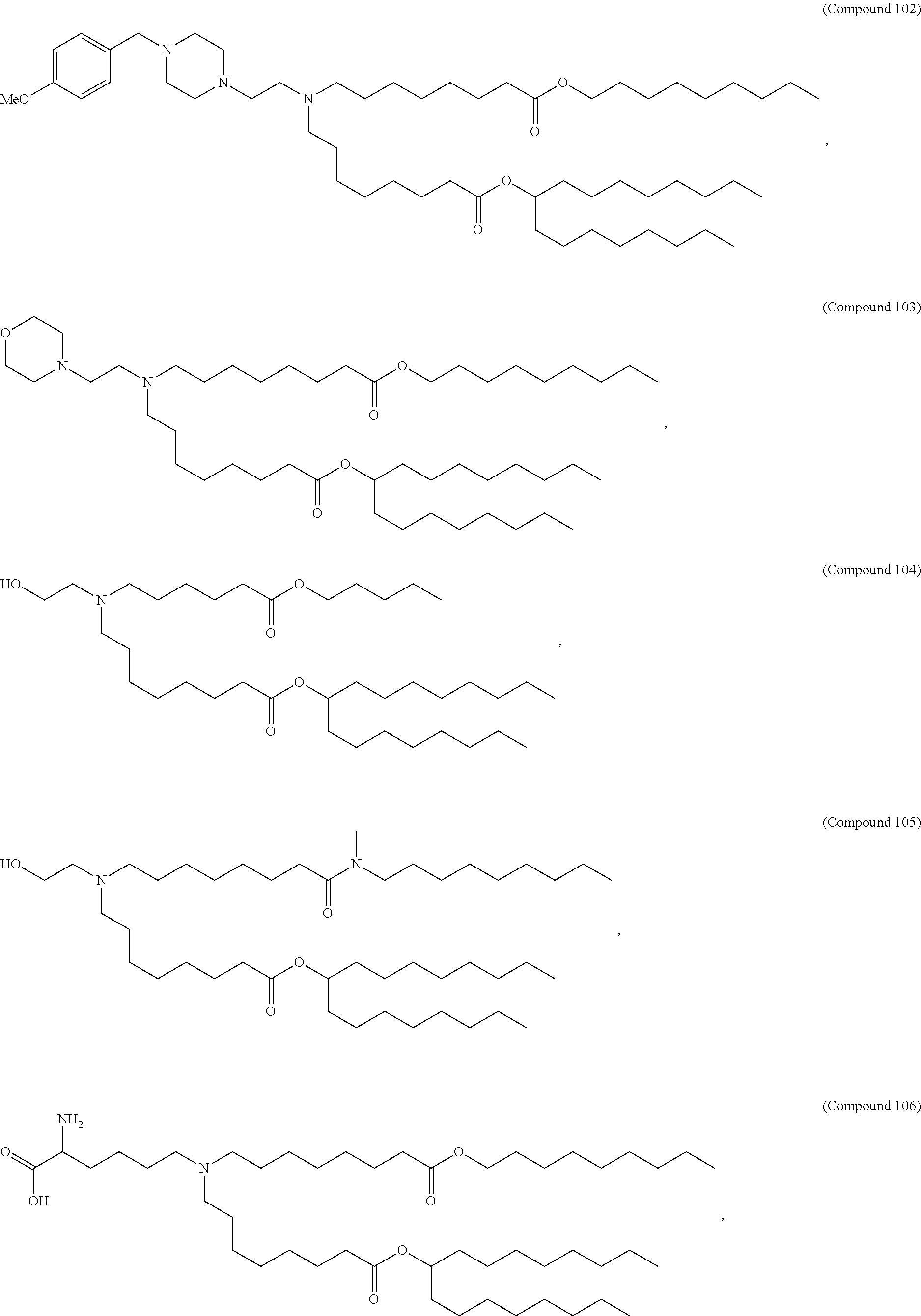 [SMILES: CCCCCCCCCOC(=O)CCCCCCCN(CCCCCCCC(=O)OC(CCCCCCCC)CCCCCCCC)CCN1CCN(Cc2ccc(OC)cc2)CC1 ; CCCCCCCCCOC(=O)CCCCCCCN(CCCCCCCC(=O)OC(CCCCCCCC)CCCCCCCC)CCN1CCOCC1 ; CCCCCCCCC(CCCCCCCC)OC(=O)CCCCCCCN(CCO)CCCCCC(=O)OCCCCC ; CCCCCCCCCN(C)C(=O)CCCCCCCN(CCO)CCCCCCCC(=O)OC(CCCCCCCC)CCCCCCCC ; CCCCCCCCCOC(=O)CCCCCCCN(CCCCCCCC(=O)OC(CCCCCCCC)CCCCCCCC)CCCCC(N)C(=O)O]

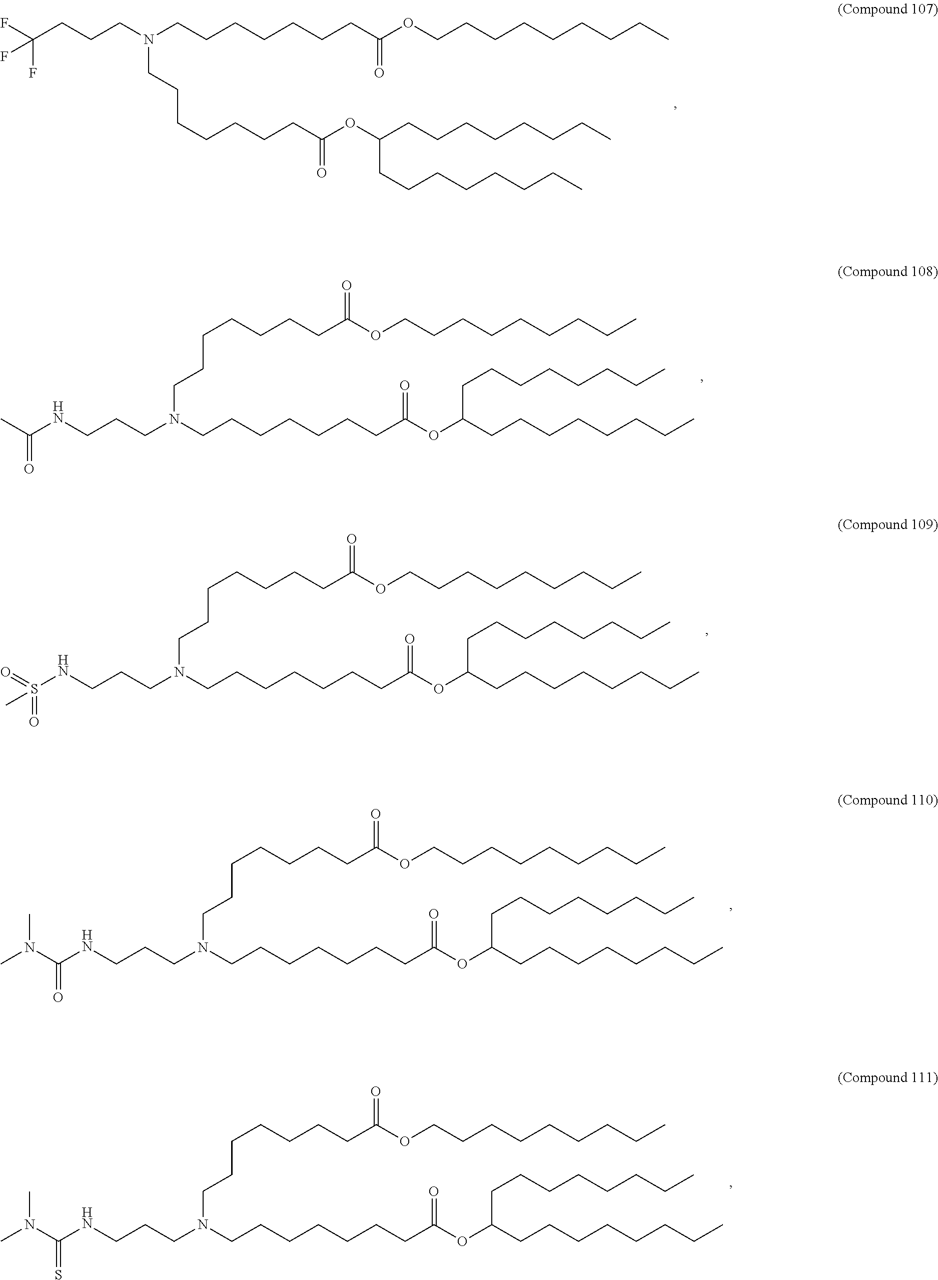

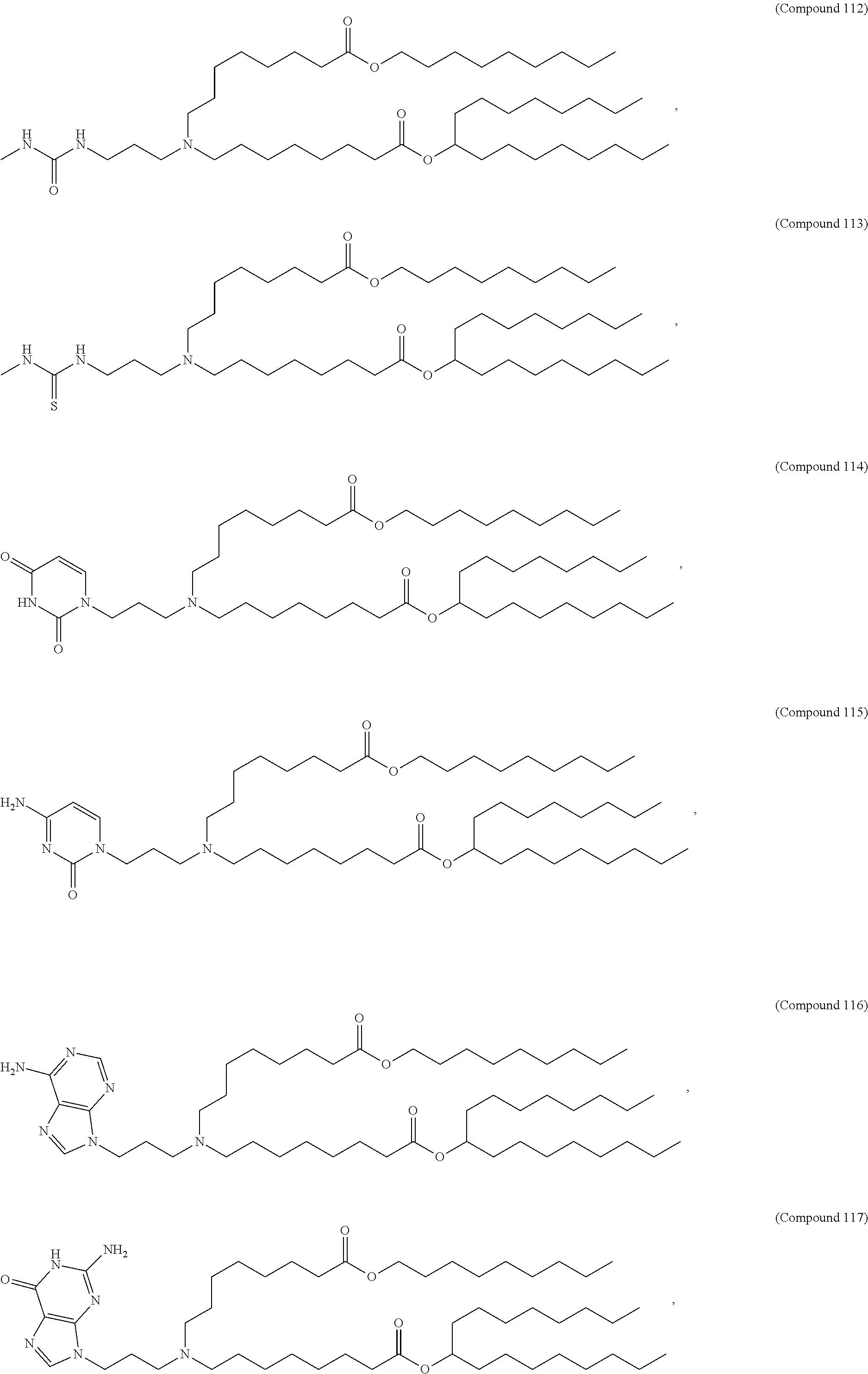

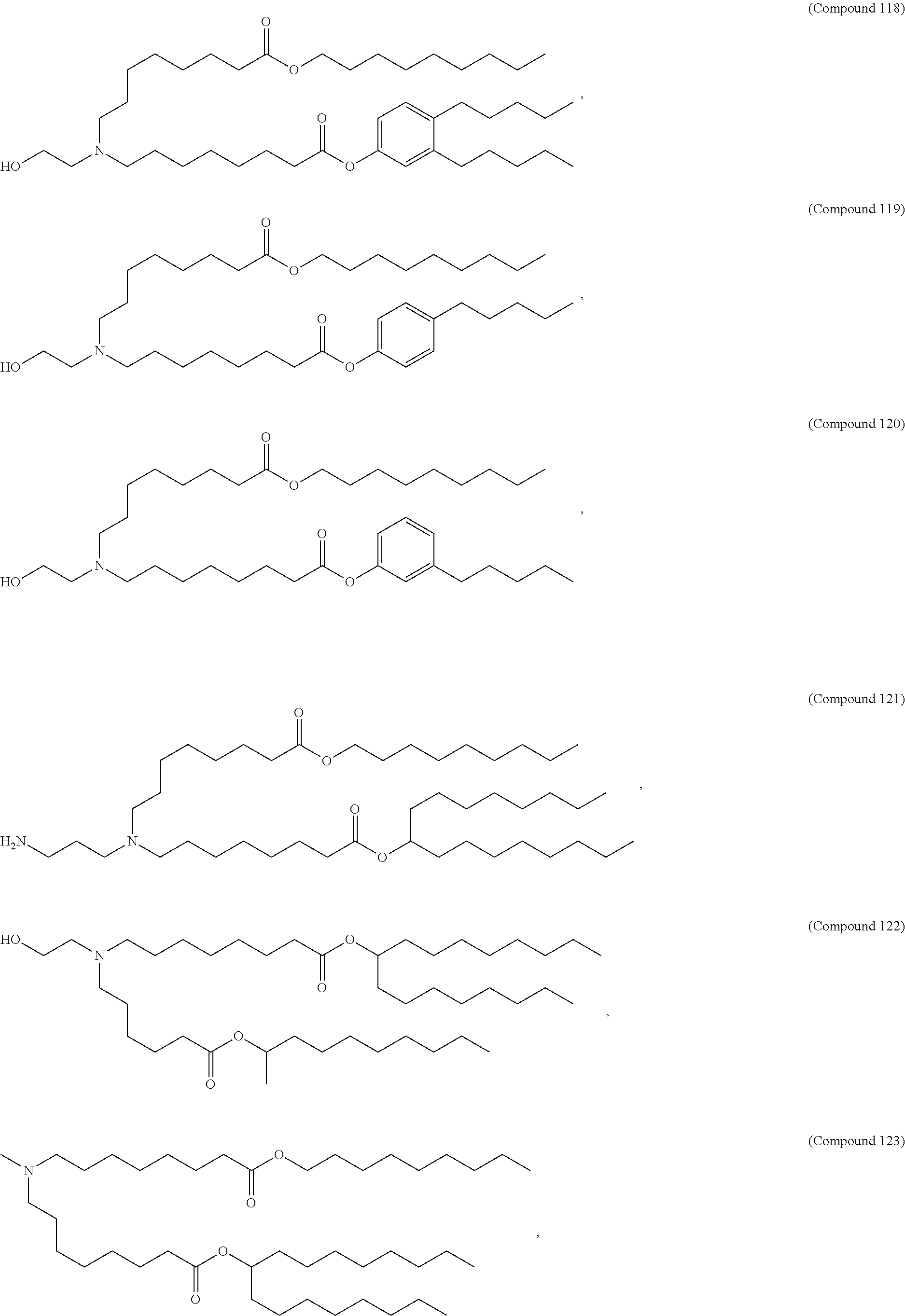

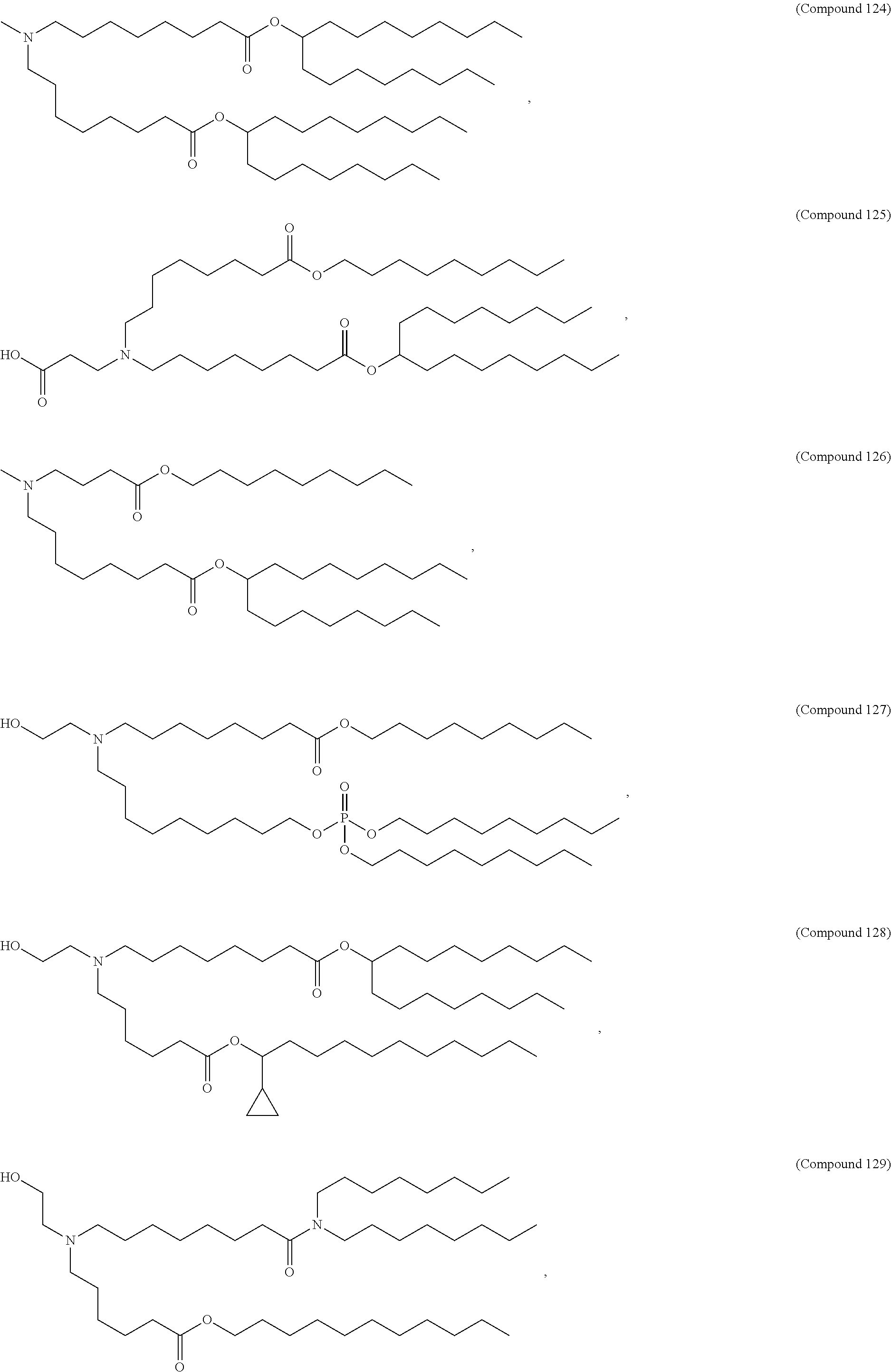

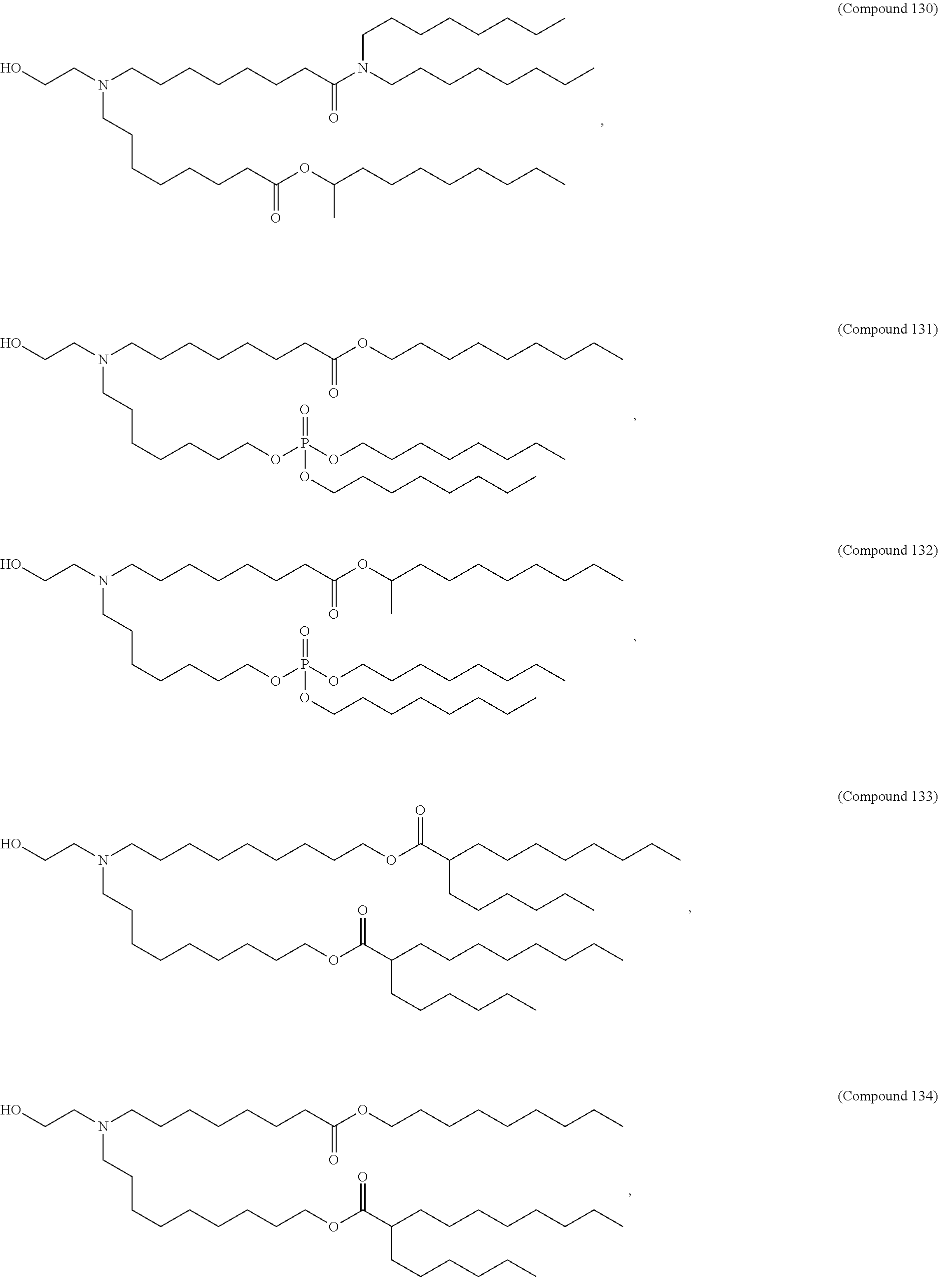 [SMILES: CCCCCCCCC(C)OC(=O)CCCCCCCN(CCO)CCCCCCCC(=O)N(CCCCCCCC)CCCCCCCC ; CCCCCCCCCOC(=O)CCCCCCCN(CCO)CCCCCCCOP(=O)(OCCCCCCCC)OCCCCCCCC ; CCCCCCCCOP(=O)(OCCCCCCCC)OCCCCCCCN(CCO)CCCCCCCC(=O)OC(C)CCCCCCCC ; CCCCCCCCC(CCCCCC)C(=O)OCCCCCCCCCN(CCO)CCCCCCCCCOC(=O)C(CCCCCC)CCCCCCCC ; CCCCCCCCCOC(=O)CCCCCCCN(CCO)CCCCCCCCCOC(=O)C(CCCCCC)CCCCCCCC]

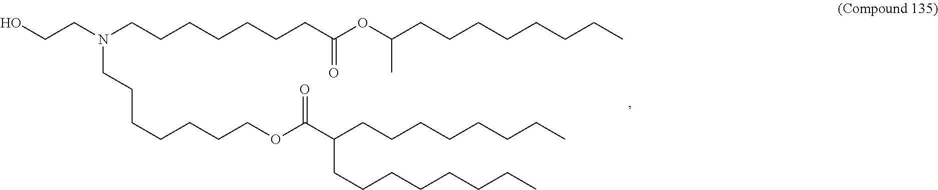 [SMILES: CCCCCCCCC(C)OC(=O)CCCCCCCN(CCO)CCCCCCCOC(=O)C(CCCCCCCC)CCCCCCCC]

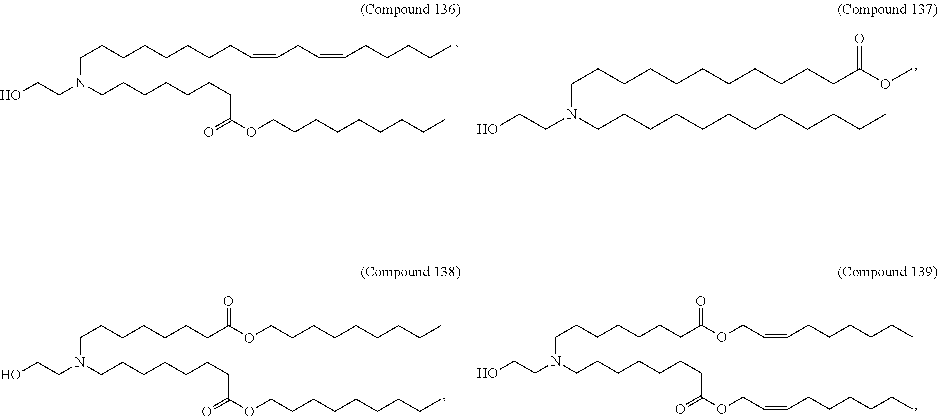 [SMILES: CCCCC/C=C\C/C=C\CCCCCCCCN(CCO)CCCCCCCC(=O)OCCCCCCCCC ; CCCCCCCCCCCCN(CCO)CCCCCCCCCCCC(=O)OC ; CCCCCCCCCOC(=O)CCCCCCCN(CCO)CCCCCCCC(=O)OCCCCCCCCC ; CCCCCC/C=C\COC(=O)CCCCCCCN(CCO)CCCCCCCC(=O)OC/C=C\CCCCCC]

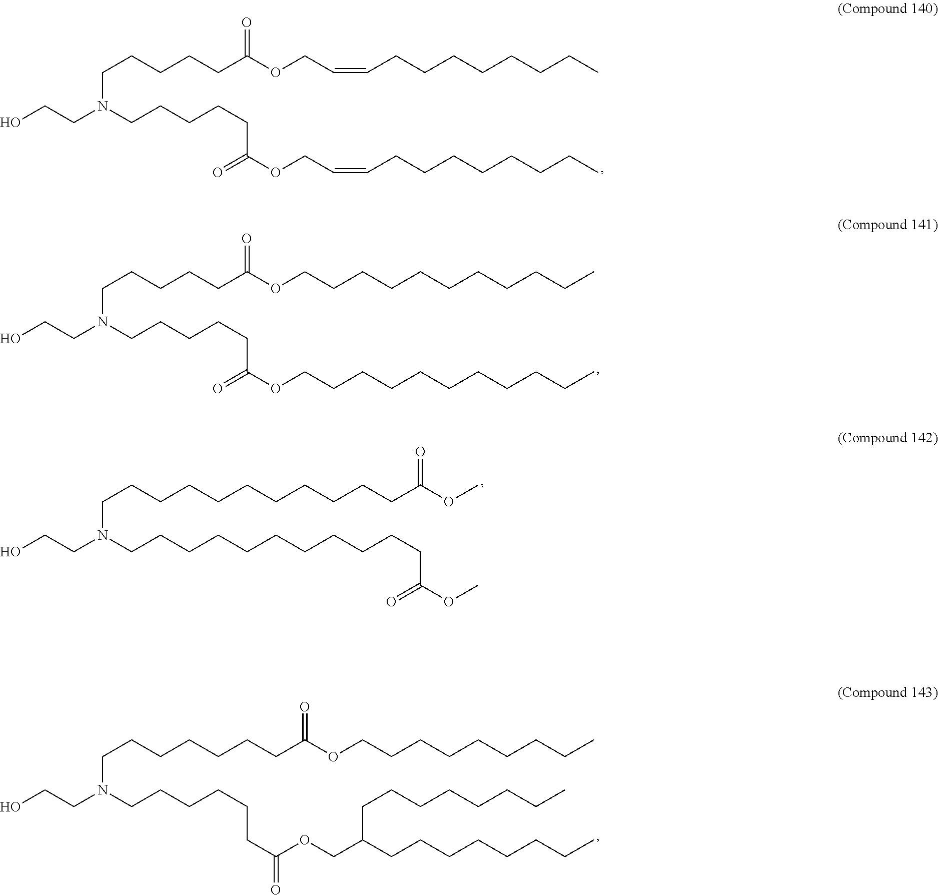 [SMILES: CCCCCCCC/C=C\COC(=O)CCCCCN(CCO)CCCCCC(=O)OC/C=C\CCCCCCCC ; CCCCCCCCCCCOC(=O)CCCCCN(CCO)CCCCCC(=O)OCCCCCCCCCCC ; COC(=O)CCCCCCCCCCCN(CCO)CCCCCCCCCCCC(=O)OC ; CCCCCCCCCOC(=O)CCCCCCCN(CCO)CCCCCCC(=O)OCC(CCCCCCCC)CCCCCCCC]

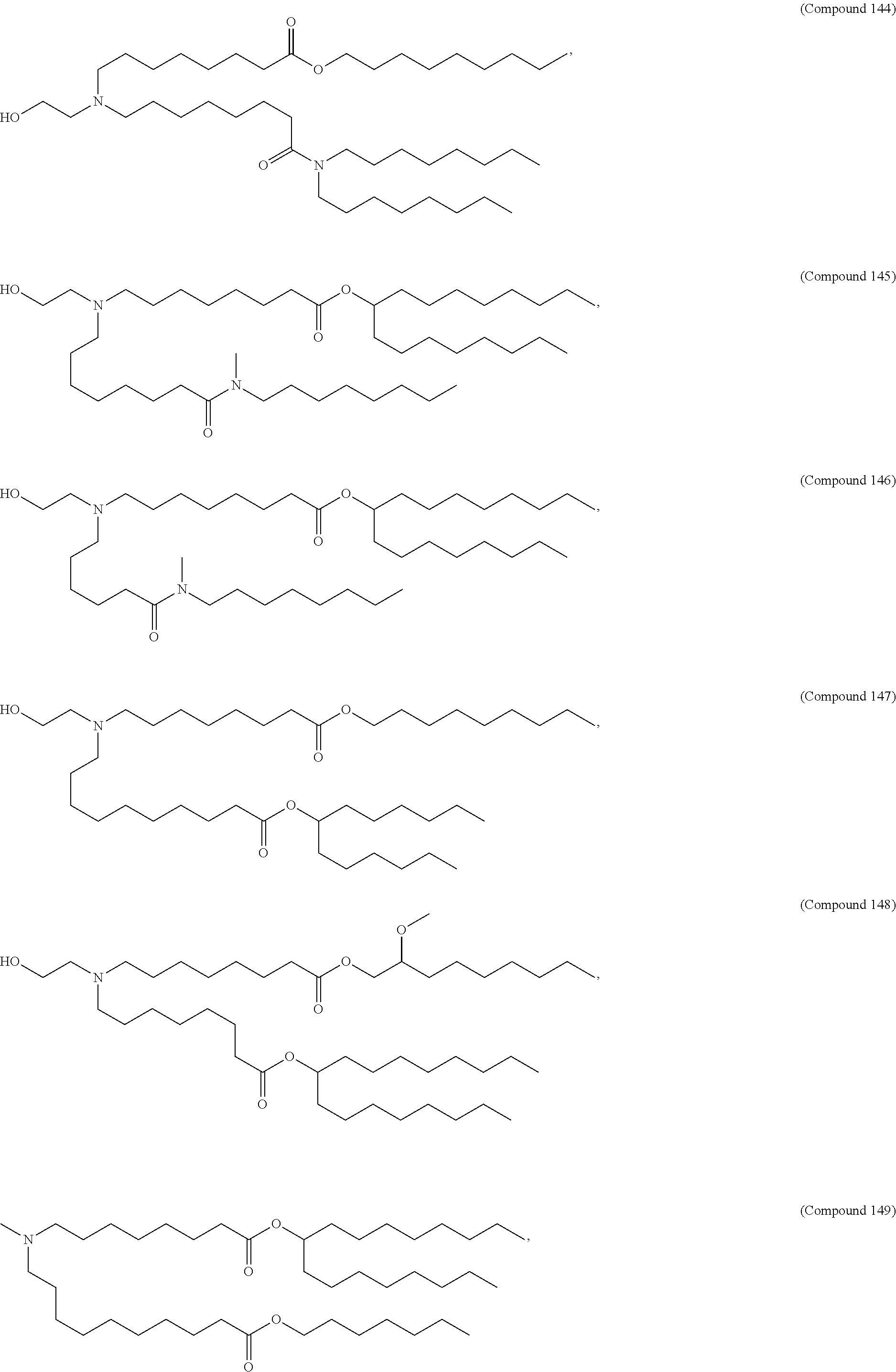

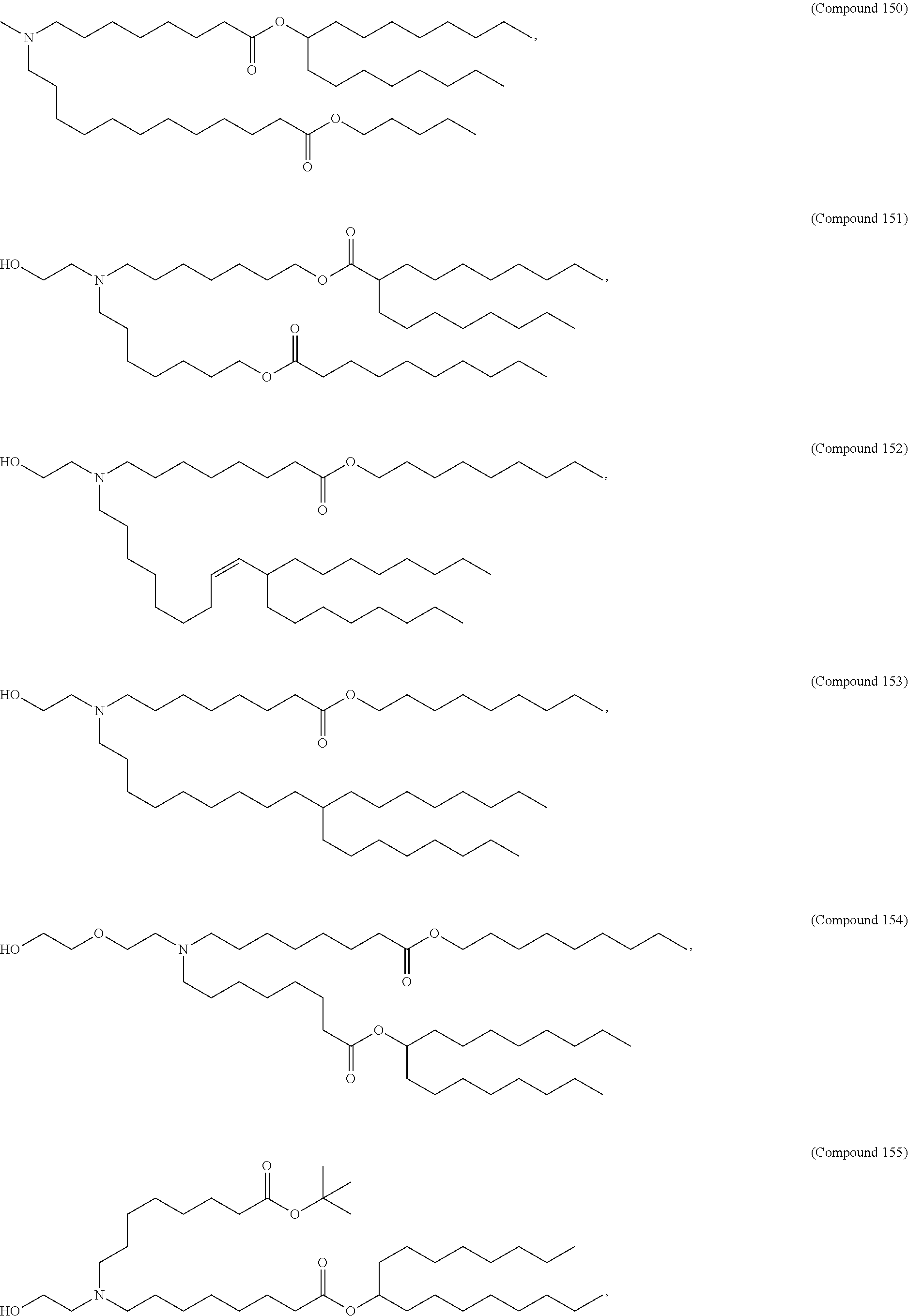 [SMILES: CCCCCCCCC(CCCCCCCC)OC(=O)CCCCCCCN(C)CCCCCCCCCCCC(=O)OCCCCC ; CCCCCCCCCC(=O)OCCCCCCCN(CCO)CCCCCCCOC(=O)C(CCCCCCCC)CCCCCCCC ; CCCCCCCCCOC(=O)CCCCCCCN(CCO)CCCCCCC/C=C\C(CCCCCCCC)CCCCCCCC ; CCCCCCCCCOC(=O)CCCCCCCN(CCO)CCCCCCCCCC(CCCCCCCC)CCCCCCCC ; CCCCCCCCCOC(=O)CCCCCCCN(CCCCCCCC(=O)OC(CCCCCCCC)CCCCCCCC)CCOCCO ; CCCCCCCCC(CCCCCCCC)OC(=O)CCCCCCCN(CCO)CCCCCCCC(=O)OC(C)(C)C]

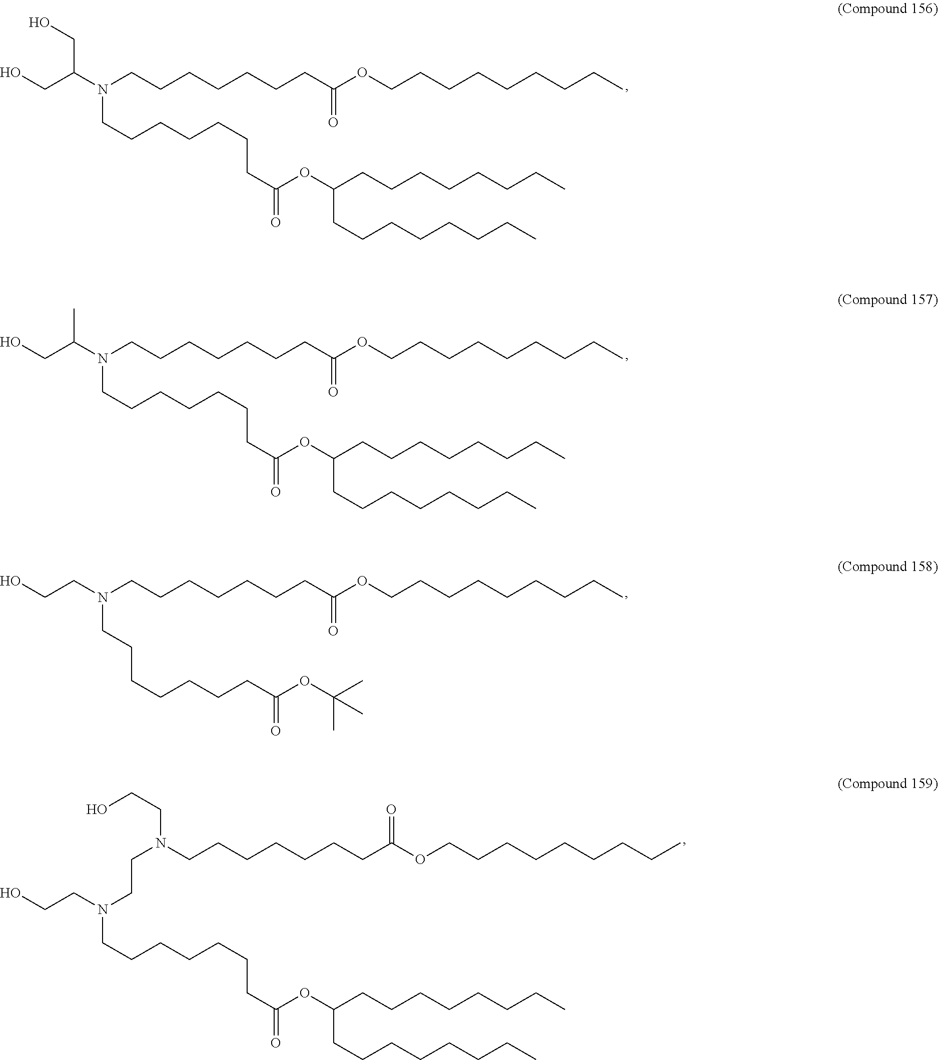 [SMILES: CCCCCCCCCOC(=O)CCCCCCCN(CCCCCCCC(=O)OC(CCCCCCCC)CCCCCCCC)C(CO)CO ; CCCCCCCCCOC(=O)CCCCCCCN(CCCCCCCC(=O)OC(CCCCCCCC)CCCCCCCC)C(C)CO ; CCCCCCCCCOC(=O)CCCCCCCN(CCO)CCCCCCCC(=O)OC(C)(C)C ; CCCCCCCCCOC(=O)CCCCCCCN(CCO)CCN(CCO)CCCCCCCC(=O)OC(CCCCCCCC)CCCCCCCC]

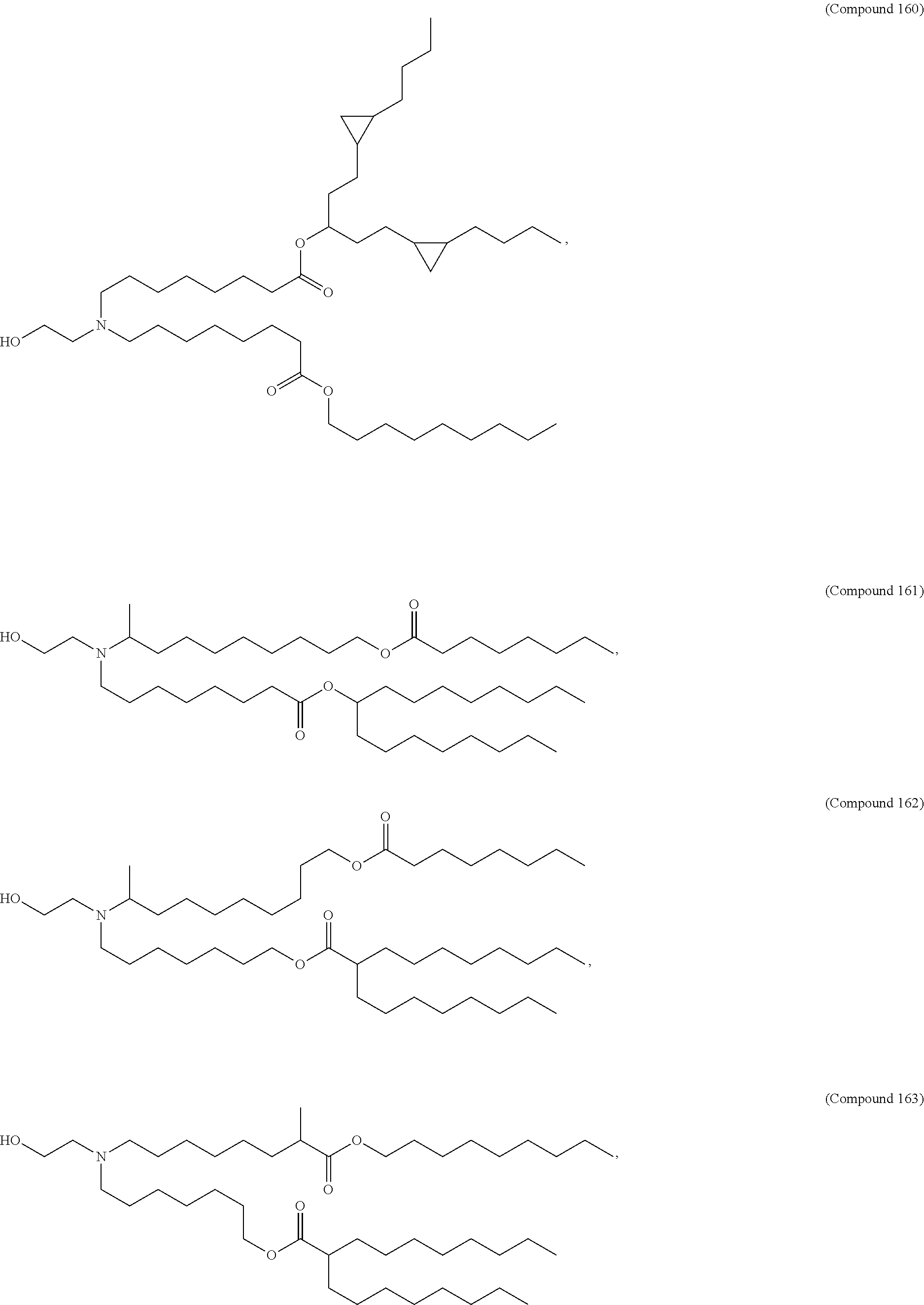 [SMILES: CCCCCCCCCOC(=O)CCCCCCCN(CCO)CCCCCCCC(=O)OC(CCC1CC1CCCC)CCC1CC1CCCC ; CCCCCCCCC(CCCCCCCC)OC(=O)CCCCCCCN(CCO)C(C)CCCCCCCCOC(=O)CCCCCCC ; CCCCCCCCC(CCCCCCCC)C(=O)OCCCCCCCN(CCO)C(C)CCCCCCCCOC(=O)CCCCCCC ; CCCCCCCCCOC(=O)C(C)CCCCCCN(CCO)CCCCCCCOC(=O)C(CCCCCCCC)CCCCCCCC]

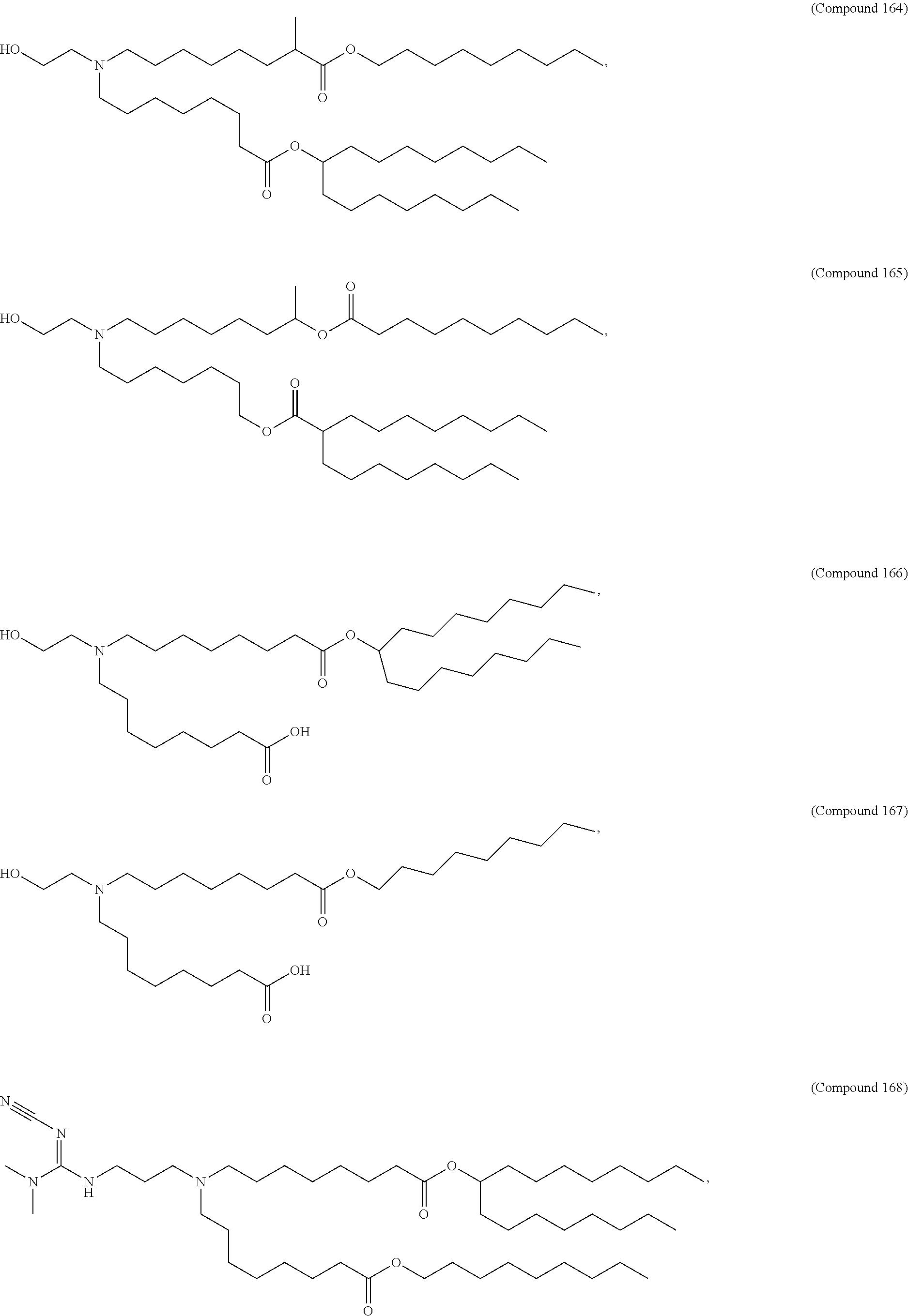 [SMILES: CCCCCCCCCOC(=O)C(C)CCCCCCN(CCO)CCCCCCCC(=O)OC(CCCCCCCC)CCCCCCCC ; CCCCCCCCCC(=O)OC(C)CCCCCCN(CCO)CCCCCCCOC(=O)C(CCCCCCCC)CCCCCCCC ; CCCCCCCCC(CCCCCCCC)OC(=O)CCCCCCCN(CCO)CCCCCCCC(=O)O ; CCCCCCCCCOC(=O)CCCCCCCN(CCO)CCCCCCCC(=O)O ; CCCCCCCCCOC(=O)CCCCCCCN(CCCCCCCC(=O)OC(CCCCCCCC)CCCCCCCC)CCCN/C(=N/C#N)N(C)C]

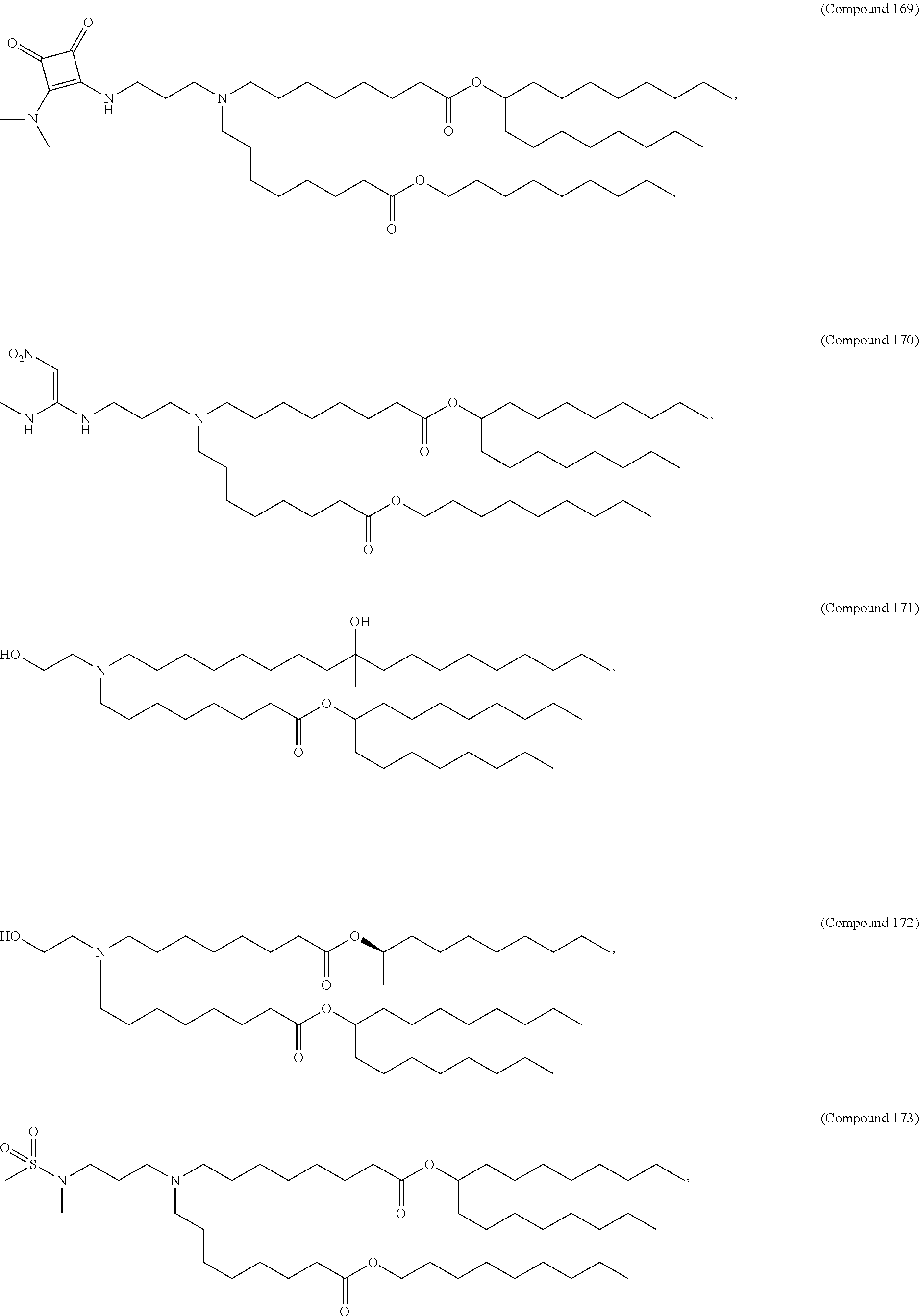

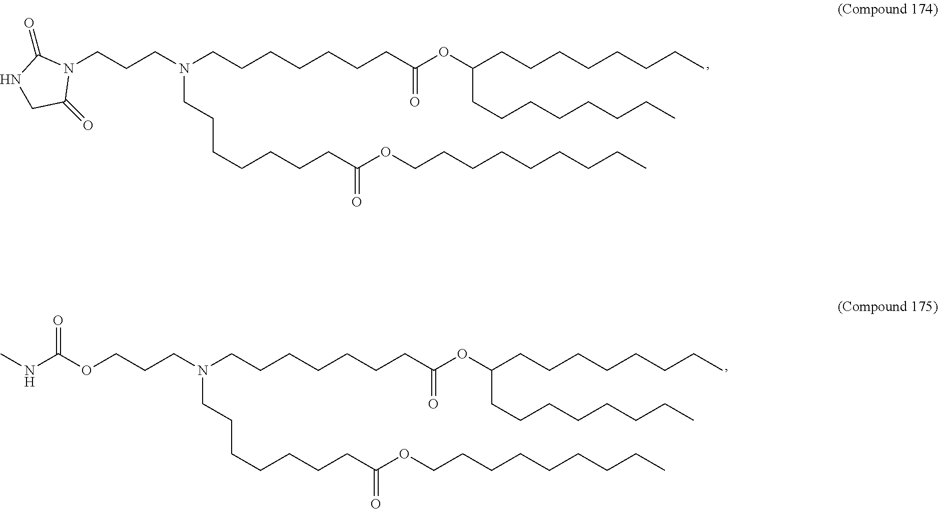

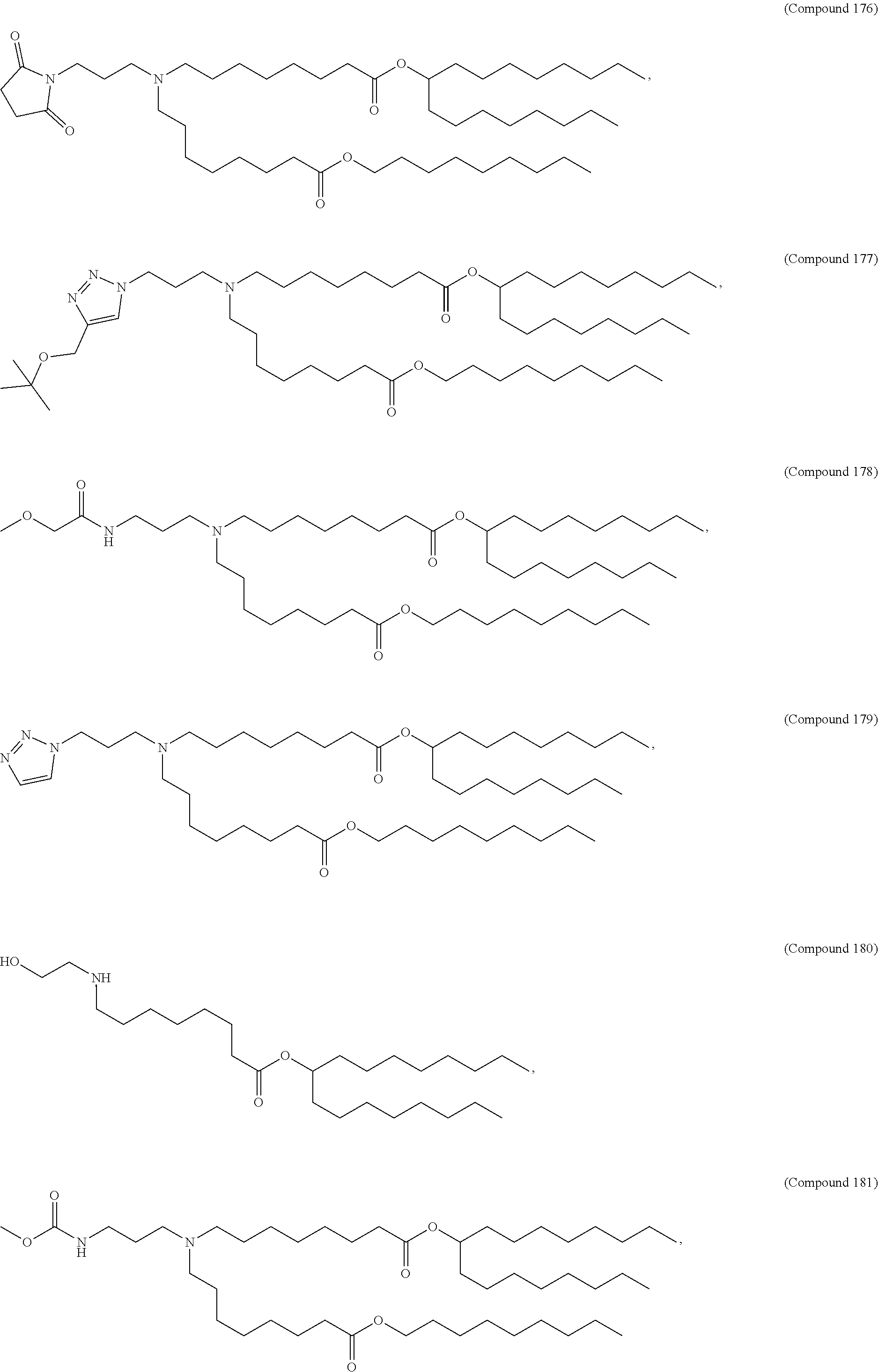

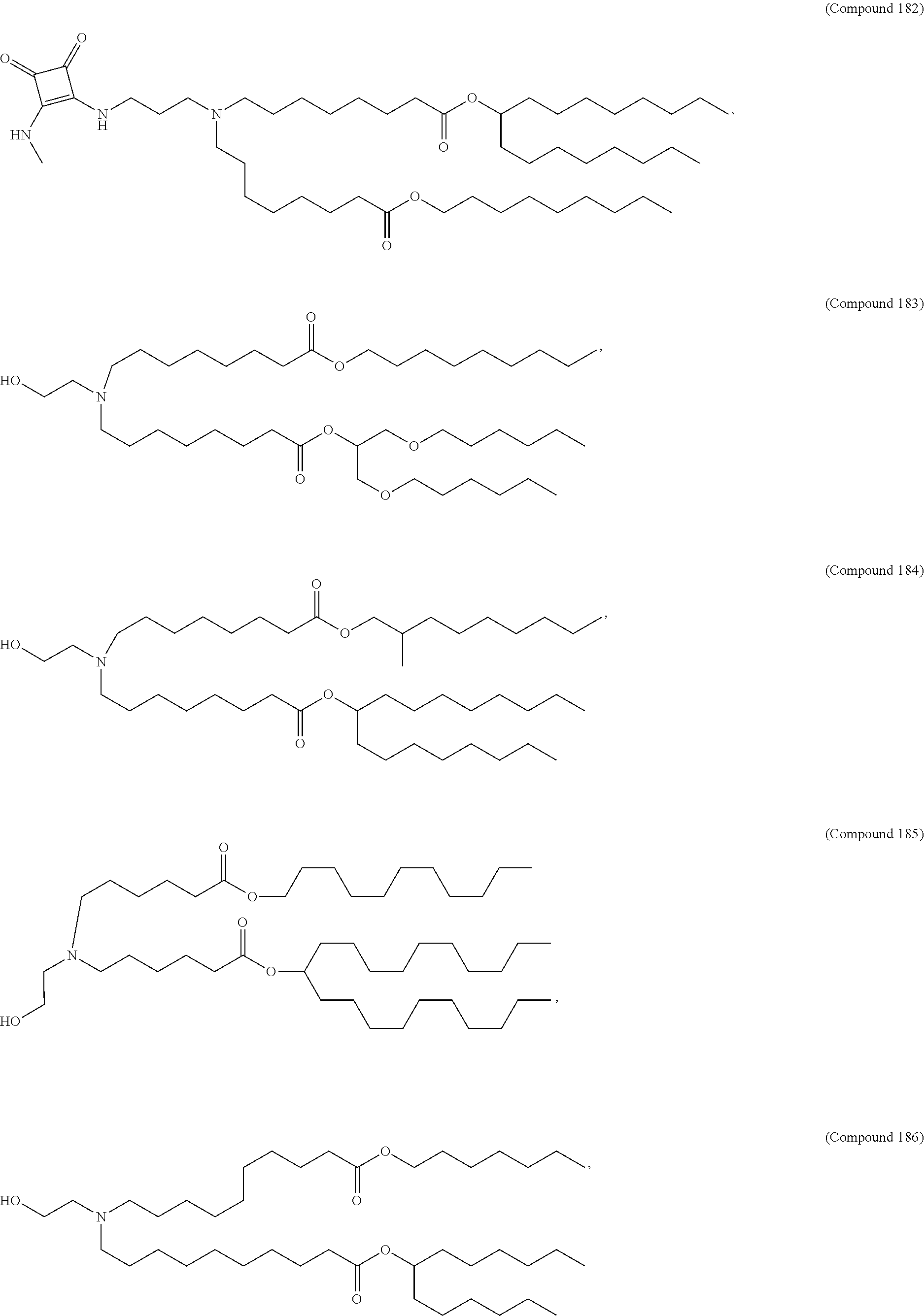 [SMILES: CCCCCCCCCOC(=O)CCCCCCCN(CCCCCCCC(=O)OC(CCCCCCCC)CCCCCCCC)CCCNc1c(NC)c(=O)c1=O ; CCCCCCCCCOC(=O)CCCCCCCN(CCO)CCCCCCCC(=O)OC(COCCCCCC)COCCCCCC ; CCCCCCCCC(CCCCCCCC)OC(=O)CCCCCCCN(CCO)CCCCCCCC(=O)OCC(C)CCCCCCC ; CCCCCCCCCCCOC(=O)CCCCCN(CCO)CCCCCC(=O)OC(CCCCCCCCCC)CCCCCCCCCC ; CCCCCCCOC(=O)CCCCCCCCCN(CCO)CCCCCCCCCC(=O)OC(CCCCCC)CCCCCC]

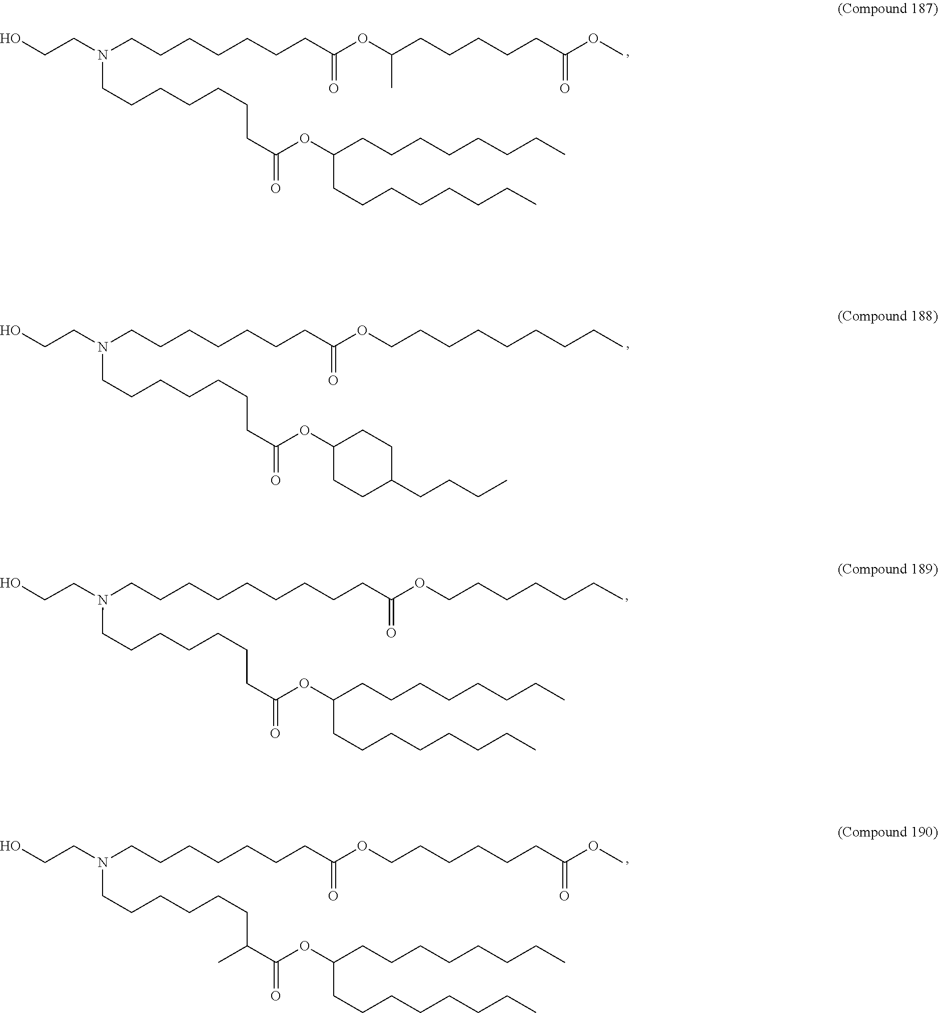 [SMILES: CCCCCCCCC(CCCCCCCC)OC(=O)CCCCCCCN(CCO)CCCCCCCC(=O)OC(C)CCCCCC(=O)OC ; CCCCCCCCCOC(=O)CCCCCCCN(CCO)CCCCCCCC(=O)OC1CCC(CCCC)CC1 ; CCCCCCCCC(CCCCCCCC)OC(=O)CCCCCCCN(CCO)CCCCCCCCCC(=O)OCCCCCCC ; CCCCCCCCC(CCCCCCCC)OC(=O)C(C)CCCCCCN(CCO)CCCCCCCC(=O)OCCCCCCC(=O)OC]

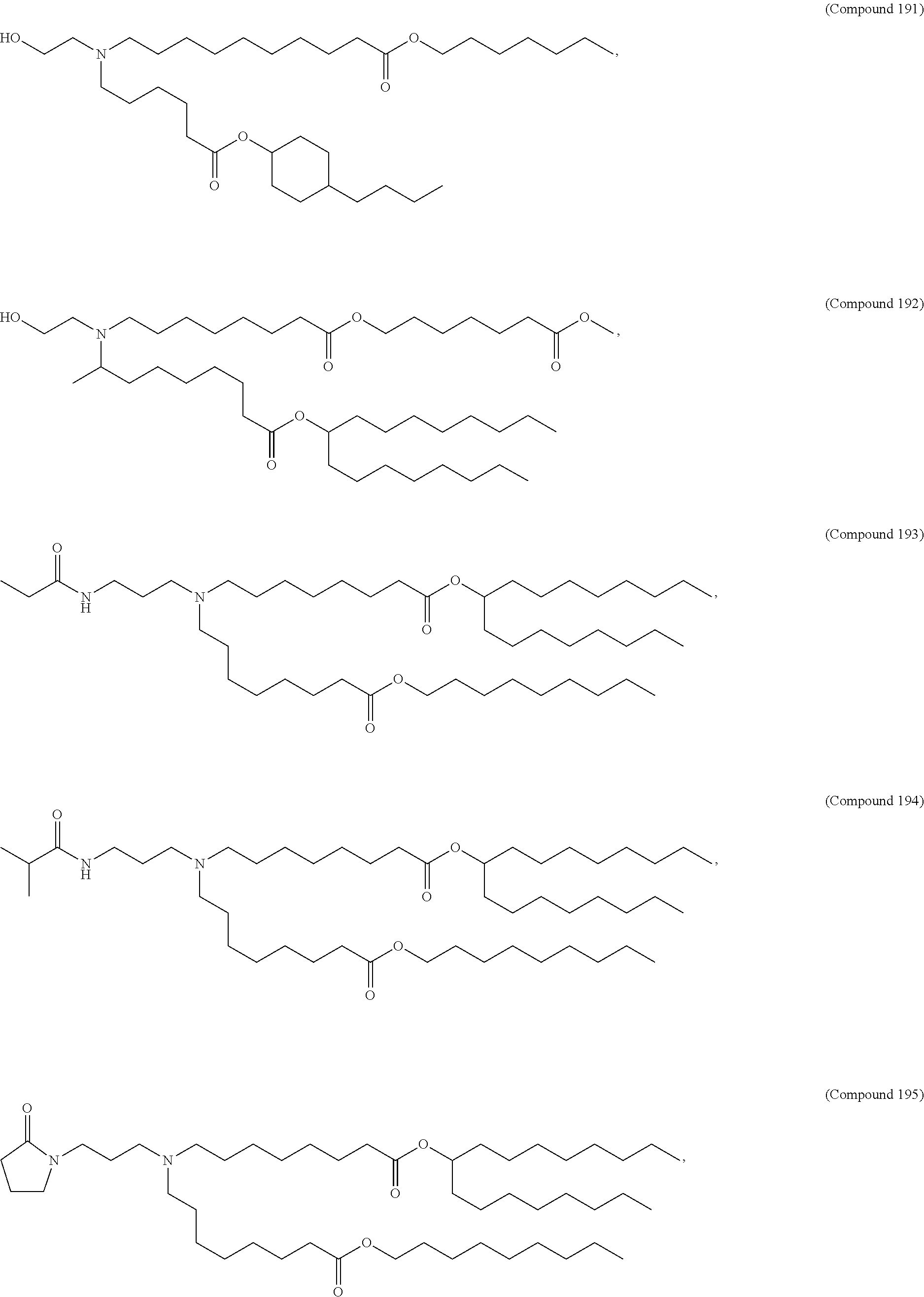

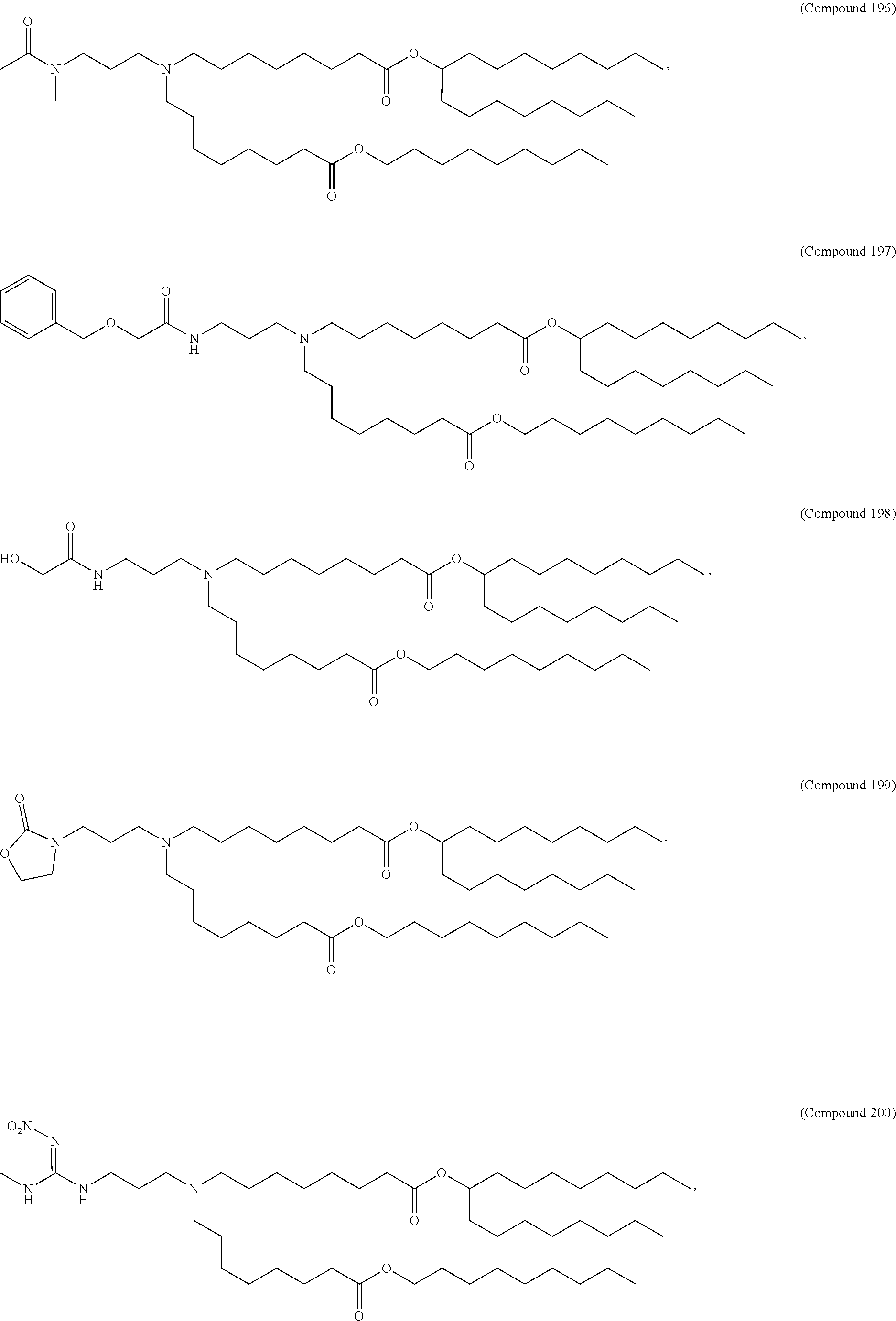

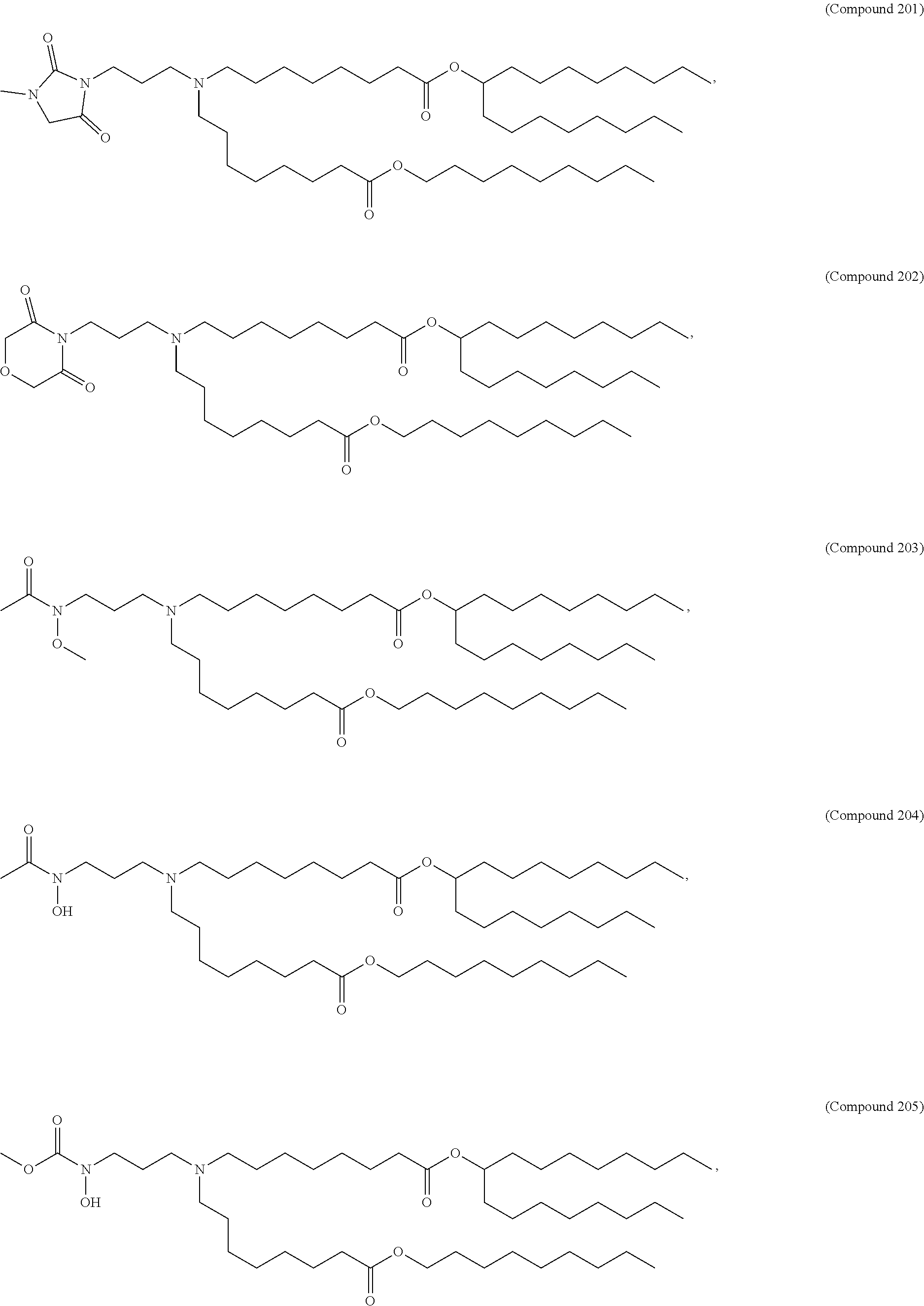

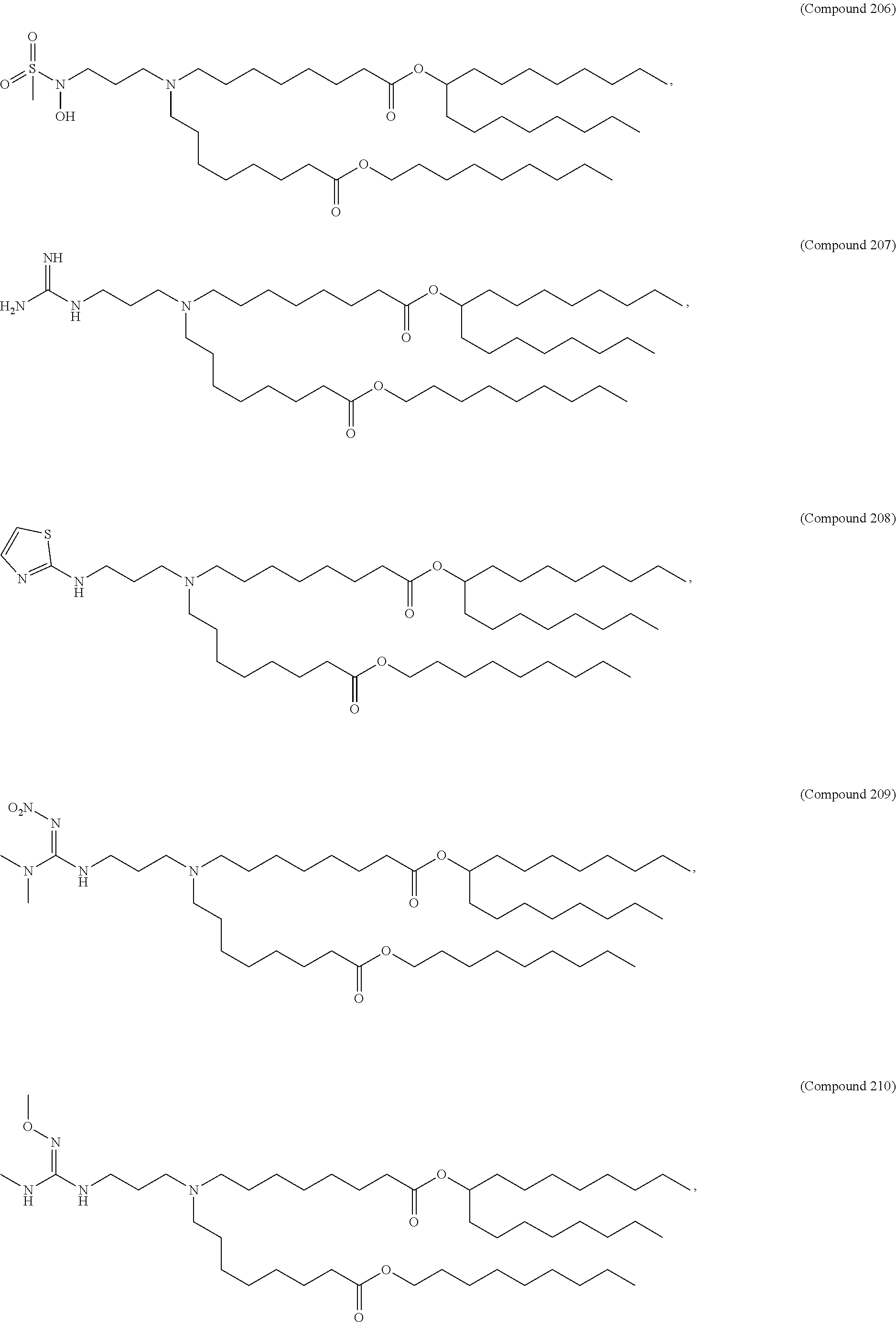

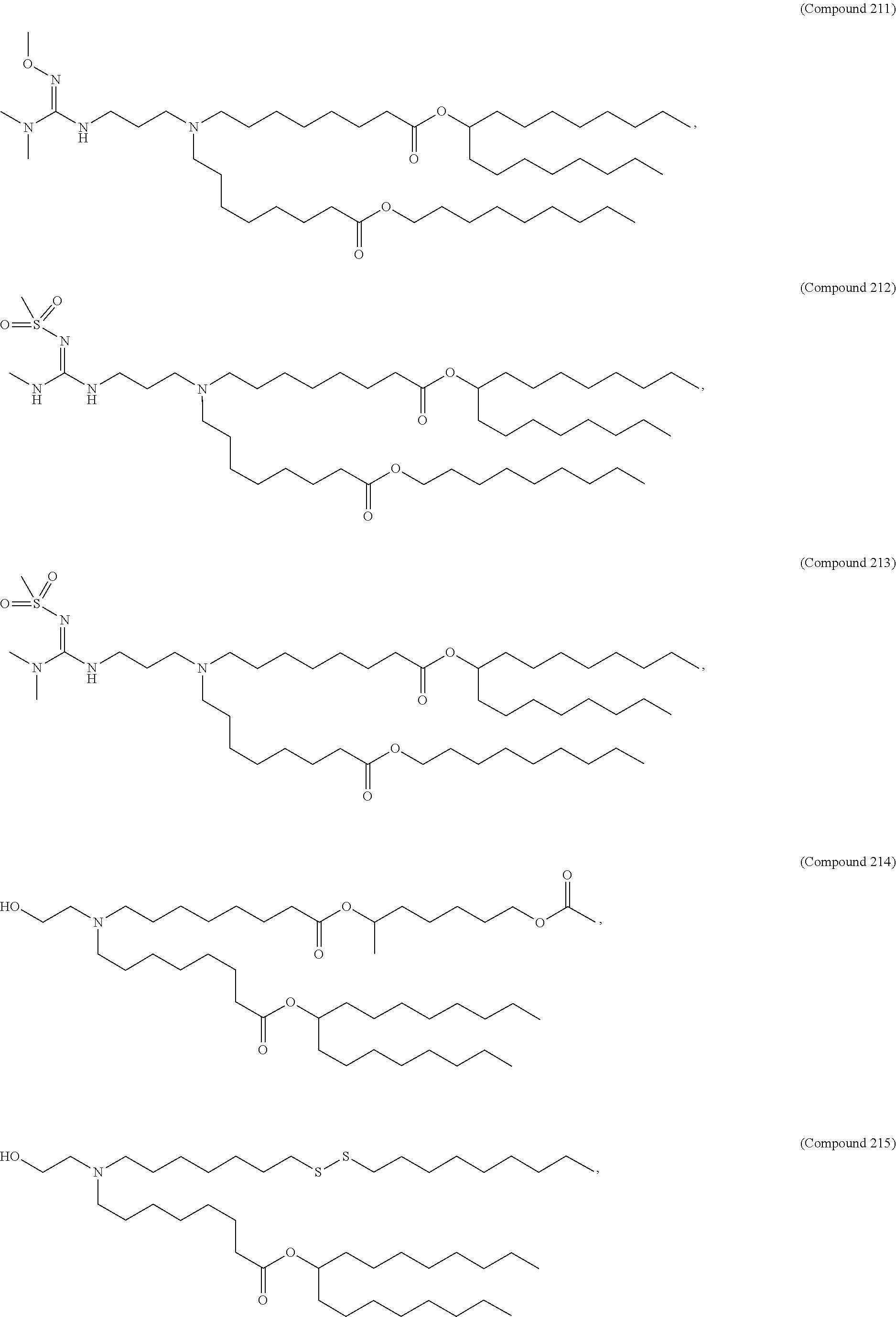 [SMILES: CCCCCCCCCOC(=O)CCCCCCCN(CCCCCCCC(=O)OC(CCCCCCCC)CCCCCCCC)CCCN/C(=N/OC)N(C)C ; CCCCCCCCCOC(=O)CCCCCCCN(CCCCCCCC(=O)OC(CCCCCCCC)CCCCCCCC)CCCN/C(=N/S(C)(=O)=O)NC ; CCCCCCCCCOC(=O)CCCCCCCN(CCCCCCCC(=O)OC(CCCCCCCC)CCCCCCCC)CCCN/C(=N/S(C)(=O)=O)N(C)C ; CCCCCCCCC(CCCCCCCC)OC(=O)CCCCCCCN(CCO)CCCCCCCC(=O)OC(C)CCCCCOC(C)=O ; CCCCCCCCCSSCCCCCCCN(CCO)CCCCCCCC(=O)OC(CCCCCCCC)CCCCCCCC]

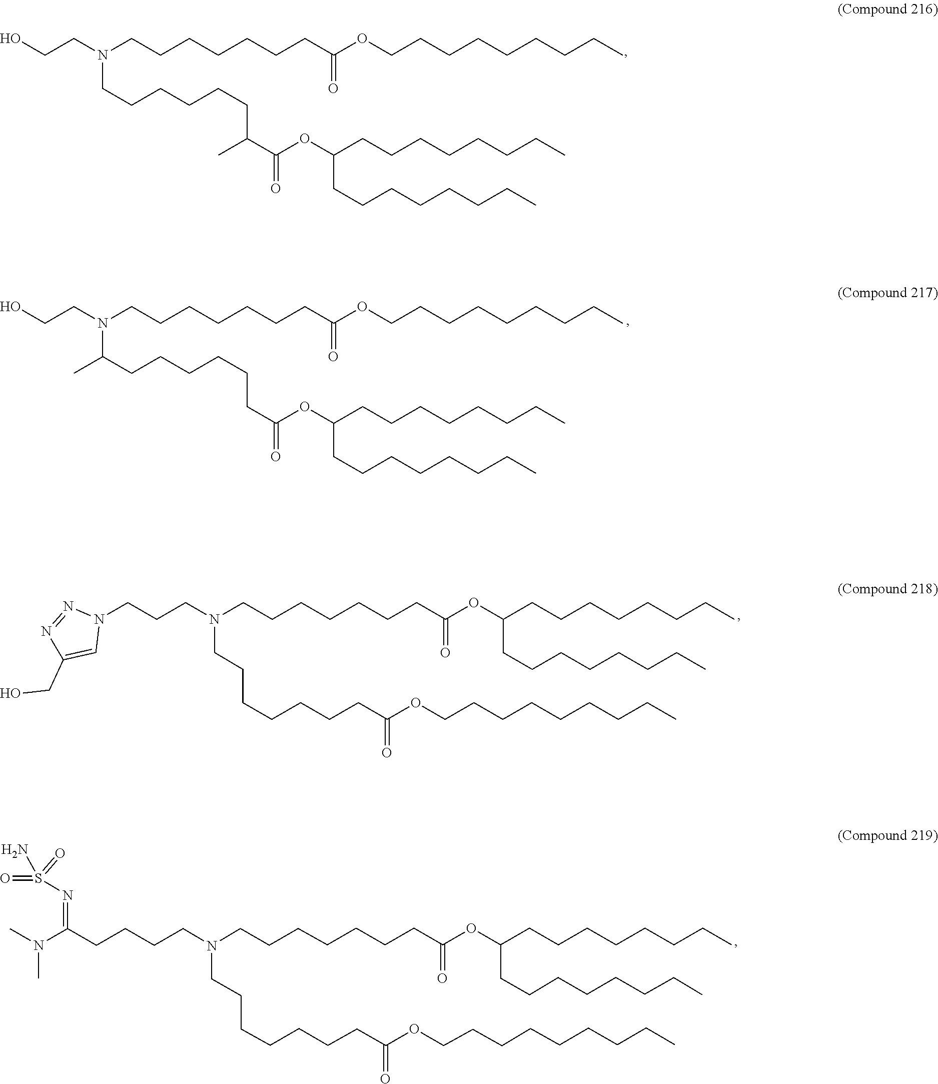 [SMILES: CCCCCCCCCOC(=O)CCCCCCCN(CCO)CCCCCCC(C)C(=O)OC(CCCCCCCC)CCCCCCCC ; CCCCCCCCCOC(=O)CCCCCCCN(CCO)C(C)CCCCCCC(=O)OC(CCCCCCCC)CCCCCCCC ; CCCCCCCCCOC(=O)CCCCCCCN(CCCCCCCC(=O)OC(CCCCCCCC)CCCCCCCC)CCCn1cc(CO)nn1 ; CCCCCCCCCOC(=O)CCCCCCCN(CCCCCCCC(=O)OC(CCCCCCCC)CCCCCCCC)CCCC/C(=N/S(N)(=O)=O)N(C)C]

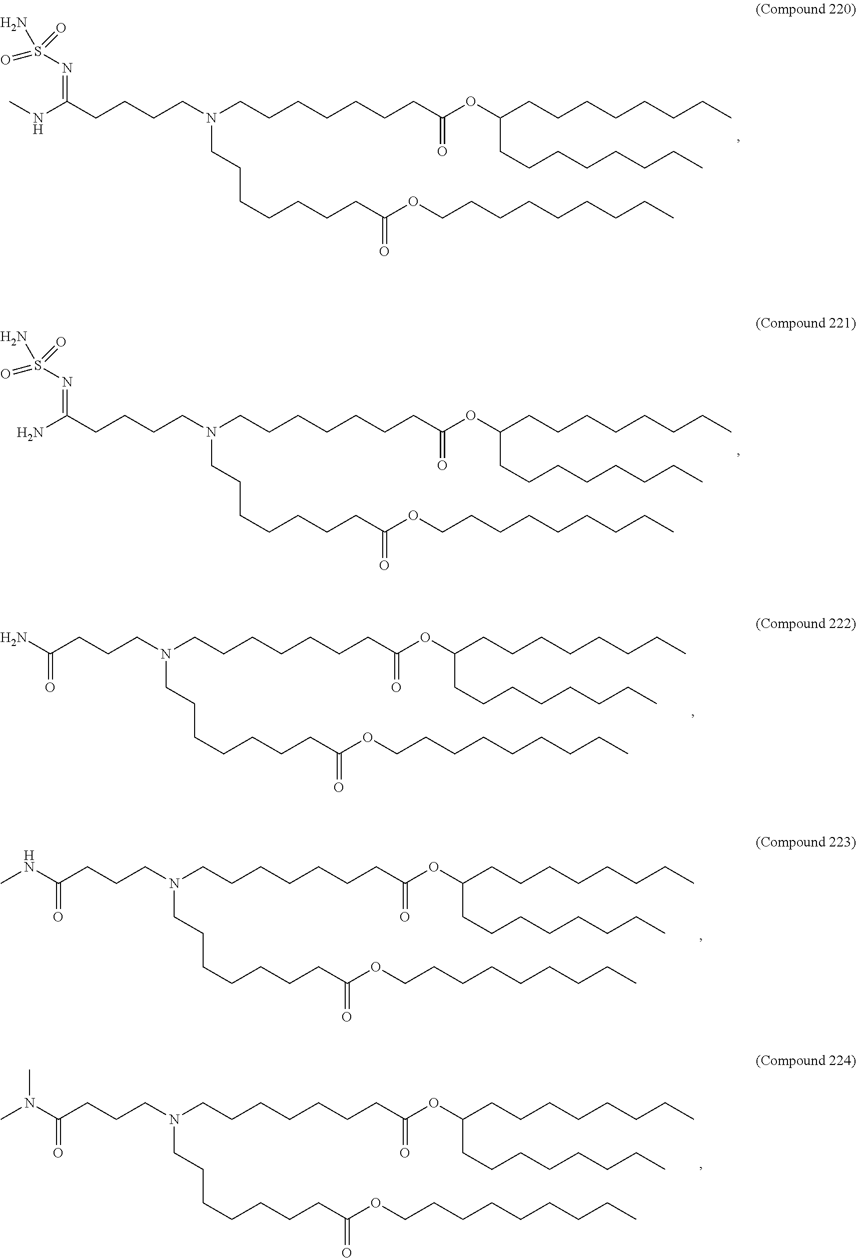

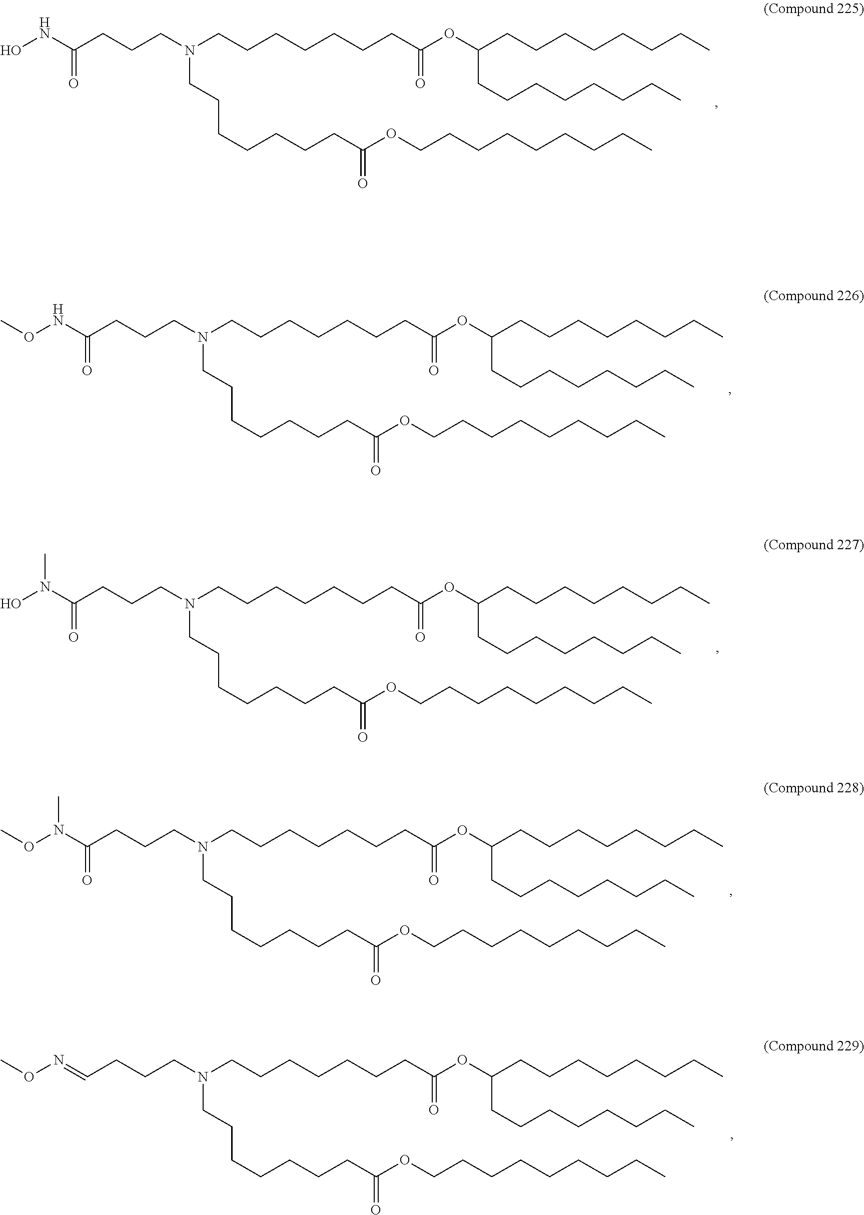

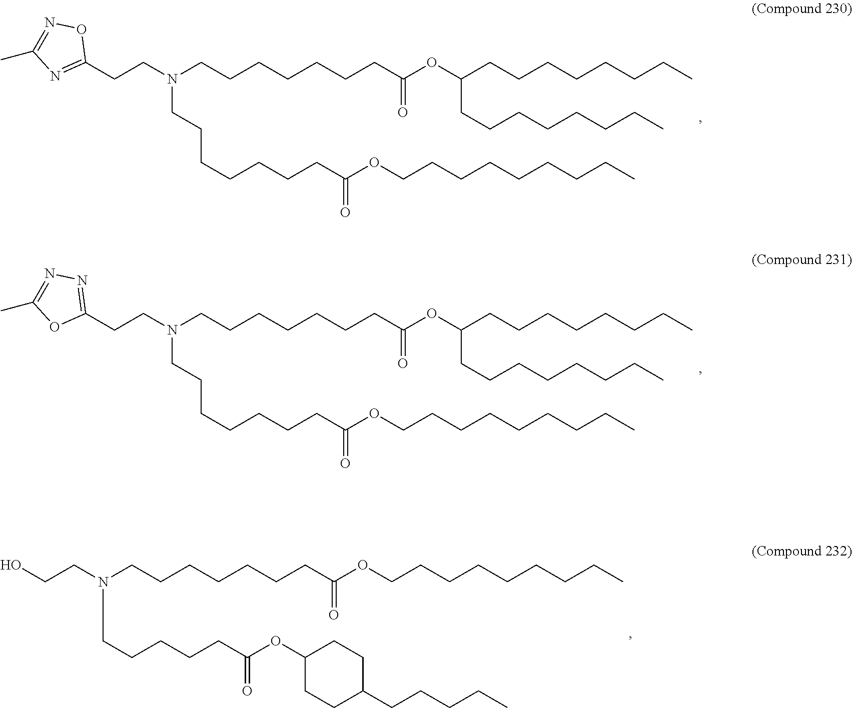

and salts and isomers thereof.

In other embodiments, the compound of Formula (I) is selected from the group consisting of Compound 1-Compound 147, or salt or stereoisomers thereof.

In some embodiments ionizable lipids including a central piperazine moiety are provided. The lipids described herein may be advantageously used in lipid nanoparticle compositions for the delivery of therapeutic and/or prophylactic agents to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent has an increased therapeutic index as compared to a corresponding formulation which comprises a reference lipid (e.g., MC3, KC2, or DLinDMA) and the same therapeutic or prophylactic agent.

In some embodiments, the delivery agent comprises a lipid compound having the formula (III)

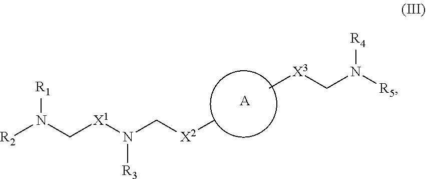

or salts or stereoisomers thereof, wherein
ring A is

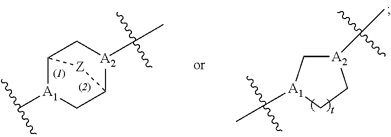

t is 1 or 2;
A₁ and A₂ are each independently selected from CH or N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₅₋₂₀ alkyl, C₅₋₂₀ alkenyl, —R″MR′, —R*YR″, —YR″, and —R*OR″;
each M is independently selected from the group consisting of —C(O)O—, —OC(O)—, —OC(O)O—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹, X², and X³ are independently selected from the group consisting of a bond, —CH₂—, —(CH₂) 2-, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH—; each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂-12 alkenyl, and H; and
each R″ is independently selected from the group consisting of C₃₋₁₂ alkyl and C₃₋₁₂ alkenyl,
wherein when ring A is then

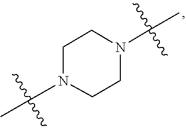 [SMILES: *N1CCN(*)CC1]

i) at least one of X¹, X², and X³ is not —CH₂—; and/or
ii) at least one of R₁, R₂, R₃, R₄, and R₅ is —R″MR′.

In some embodiments, the compound is of any of formulae (IIIa1)(IIIa6):

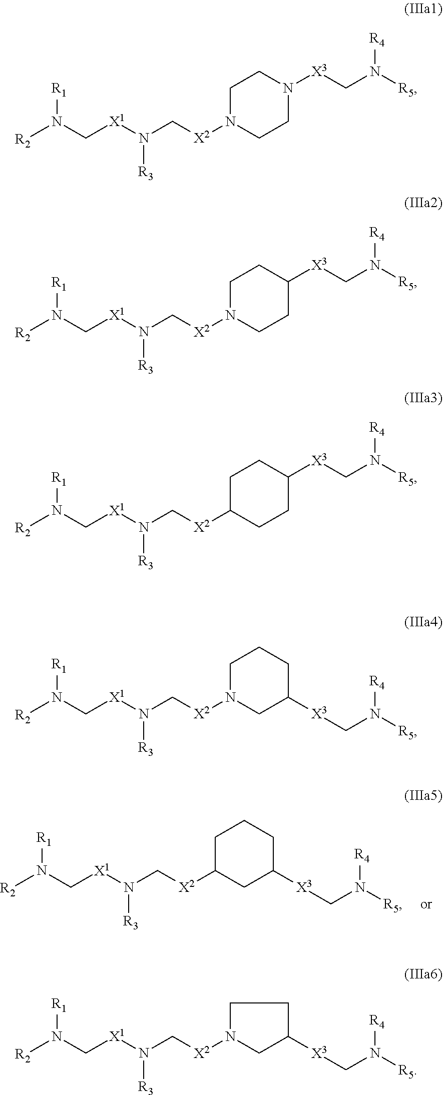

The compounds of Formula (III) or any of (IIIa1)-(IIIa6) include one or more of the following features when applicable.

In some embodiments, ring A is

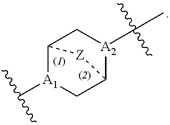

In some embodiments, ring A is

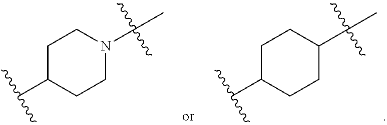 [SMILES: *C1CCN(*)CC1 ; *C1CCC(*)CC1]

In some embodiments, ring A is

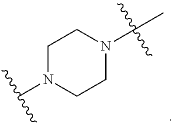 [SMILES: *N1CCN(*)CC1]

In some embodiments, ring A is

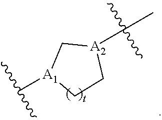

In some embodiments, ring A is

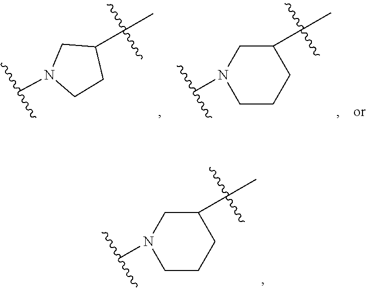 [SMILES: *C1CCN(*)C1 ; *C1CCCN(*)C1 ; *C1CCCN(*)C1]

In some embodiments, ring A is

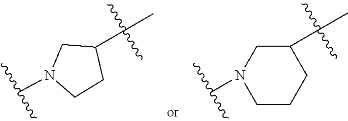 [SMILES: *C1CCN(*)C1 ; *C1CCCN(*)C1]

wherein ring, in which the N atom is connected with X².

In some embodiments, Z is CH₂.

In some embodiments, Z is absent.

In some embodiments, at least one of A₁ and A₂ is N.

In some embodiments, each of A₁ and A₂ is N.

In some embodiments, each of A₁ and A₂ is CH.

In some embodiments, A₁ is N and A₂ is CH.

In some embodiments, A₁ is CH and A₂ is N.

In some embodiments, at least one of X¹, X², and X³ is not —CH₂—. For example, in certain embodiments, X¹ is not —CH₂—. In some embodiments, at least one of X¹, x2, and X³ is —C(O)—.

In some embodiments, X² is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—.

In some embodiments, X³ is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—. In other embodiments, X³ is —CH₂—.

In some embodiments, X³ is a bond or —(CH₂)₂—.

In some embodiments, R₁ and R₂ are the same. In certain embodiments, R₁, R₂, and R₃ are the same. In some embodiments, R₄ and R₅ are the same. In certain embodiments, R₁, R₂, R₃, R₄, and R₅ are the same.

In some embodiments, at least one of R₁, R₂, R₃, R₄, and R₅ is —R″MR′. In some embodiments, at most one of R₁, R₂, R₃, R₄, and R₅ is —R″MR′. For example, at least one of R₁, R₂, and R₃ may be —R″MR′, and/or at least one of R₄ and R₅ is —R″MR′. In certain embodiments, at least one M is —C(O)O—. In some embodiments, each M is —C(O)O—. In some embodiments, at least one M is —OC(O)—. In some embodiments, each M is —OC(O)—. In some embodiments, at least one M is —OC(O)O—. In some embodiments, each M is —OC(O)O—. In some embodiments, at least one R″ is C₃ alkyl. In certain embodiments, each R″ is C₃ alkyl. In some embodiments, at least one R″ is C₅ alkyl. In certain embodiments, each R″ is C₅ alkyl. In some embodiments, at least one R″ is C₆ alkyl. In certain embodiments, each R″ is C₆ alkyl. In some embodiments, at least one R″ is C₇ alkyl. In certain embodiments, each R″ is C₇ alkyl. In some embodiments, at least one R′ is C₅ alkyl. In certain embodiments, each R′ is C₅ alkyl. In other embodiments, at least one R′ is C₁ alkyl. In certain embodiments, each R′ is C₁ alkyl. In some embodiments, at least one R′ is C₂ alkyl. In certain embodiments, each R′ is C₂ alkyl.

In some embodiments, at least one of R₁, R₂, R₃, R₄, and R₅ is C₁₂ alkyl. In certain embodiments, each of R₁, R₂, R₃, R₄, and R₅ are C₁₂ alkyl.

In certain embodiments, the compound is selected from the group consisting of:

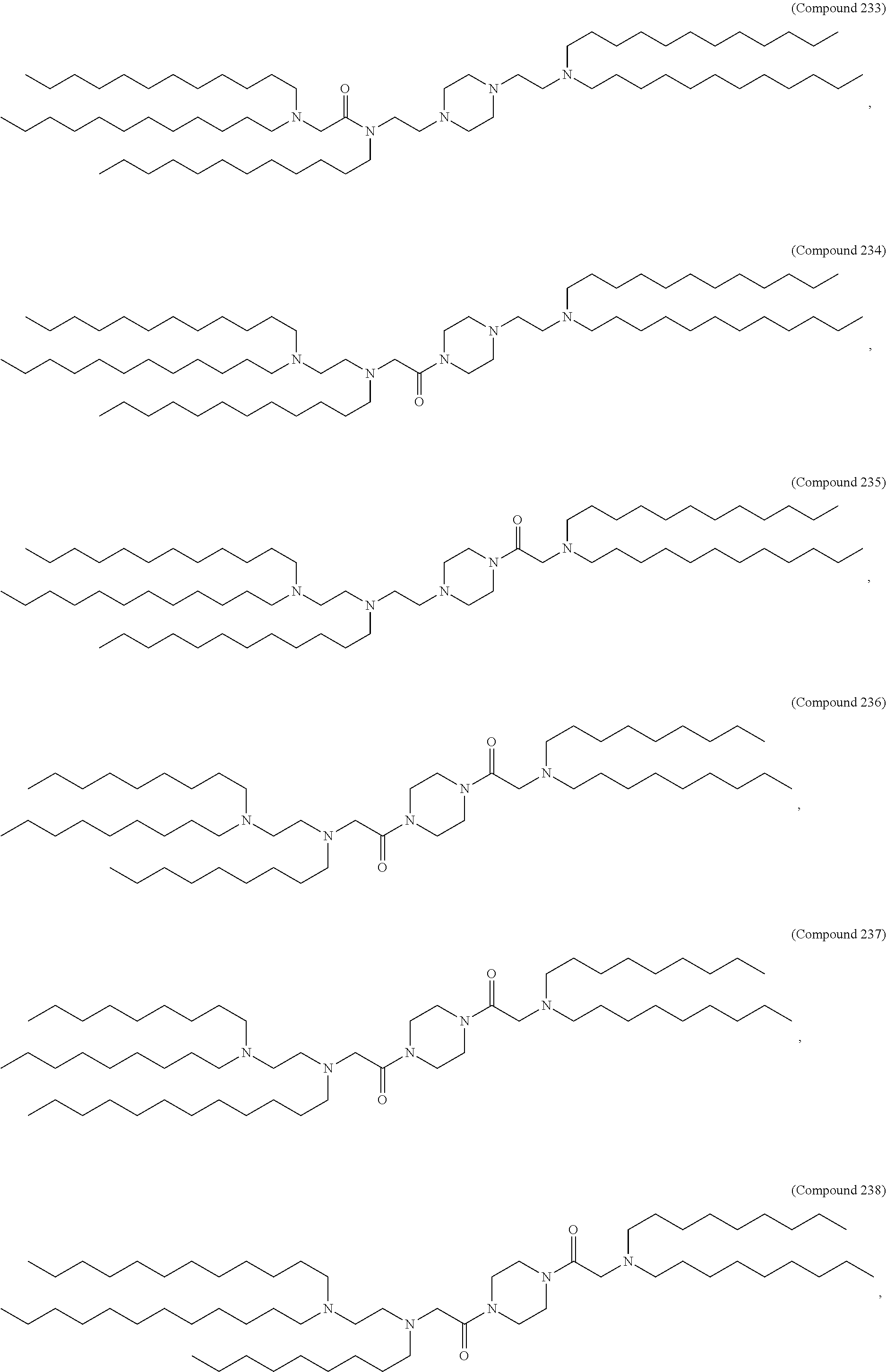

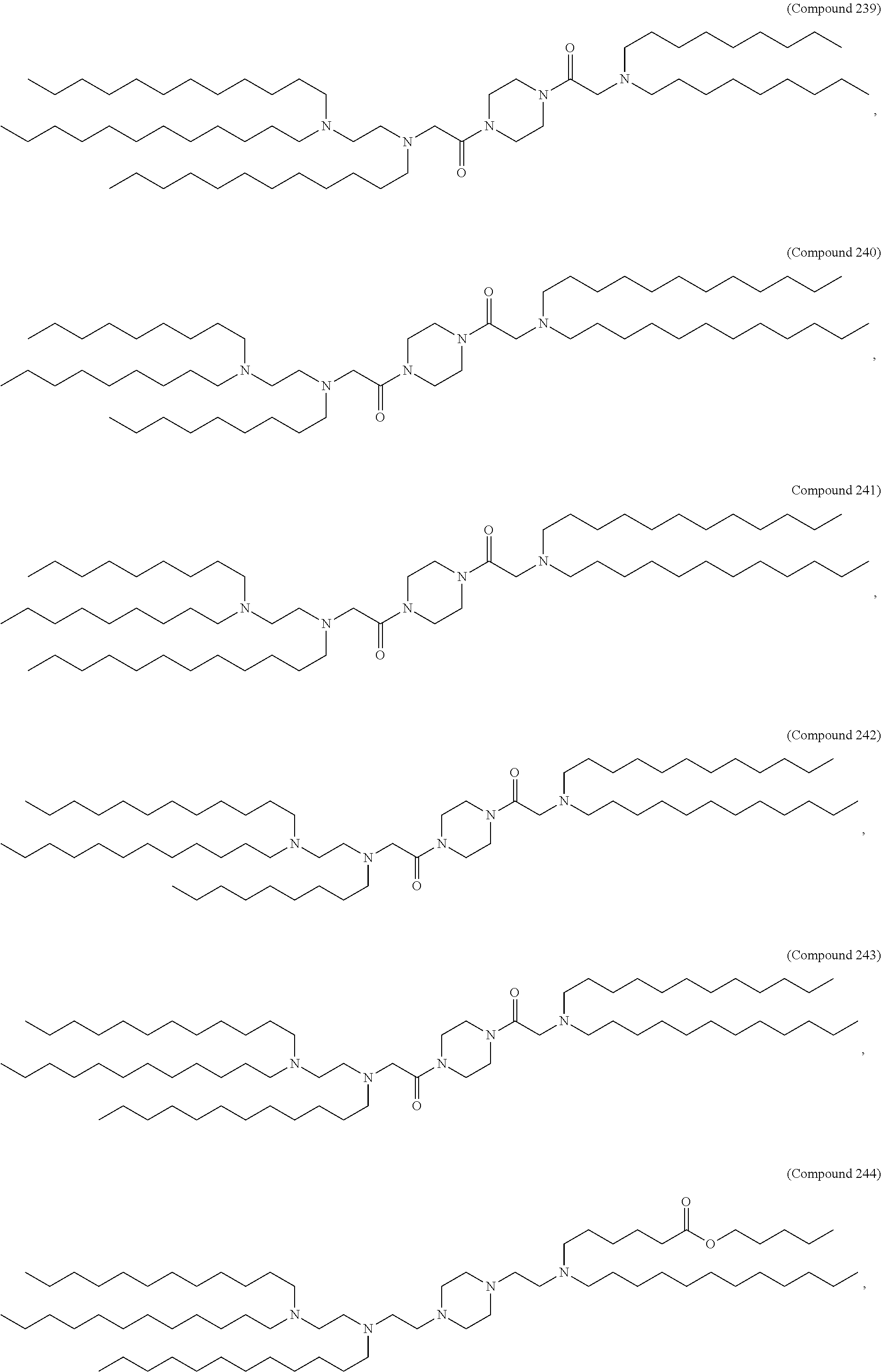

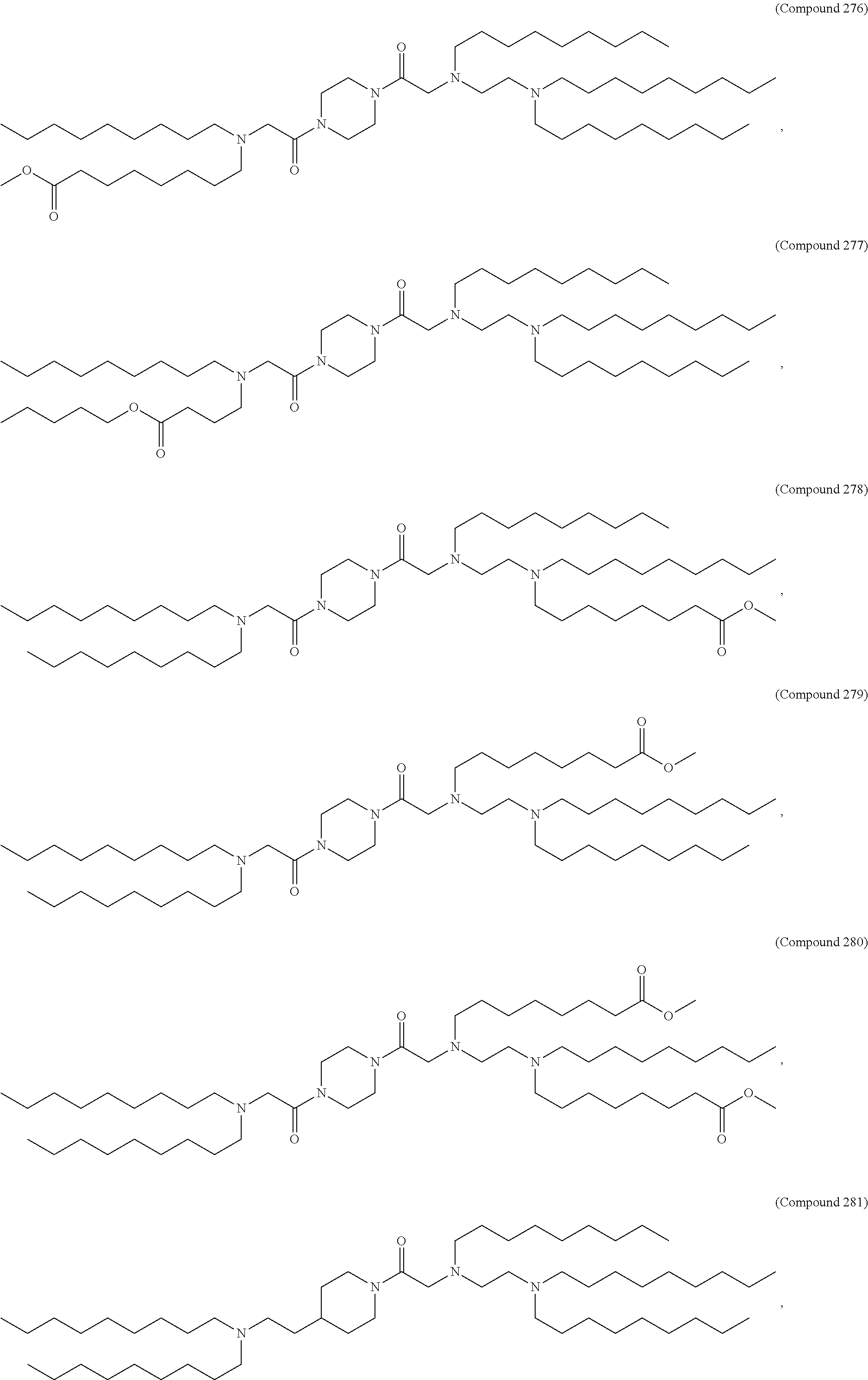

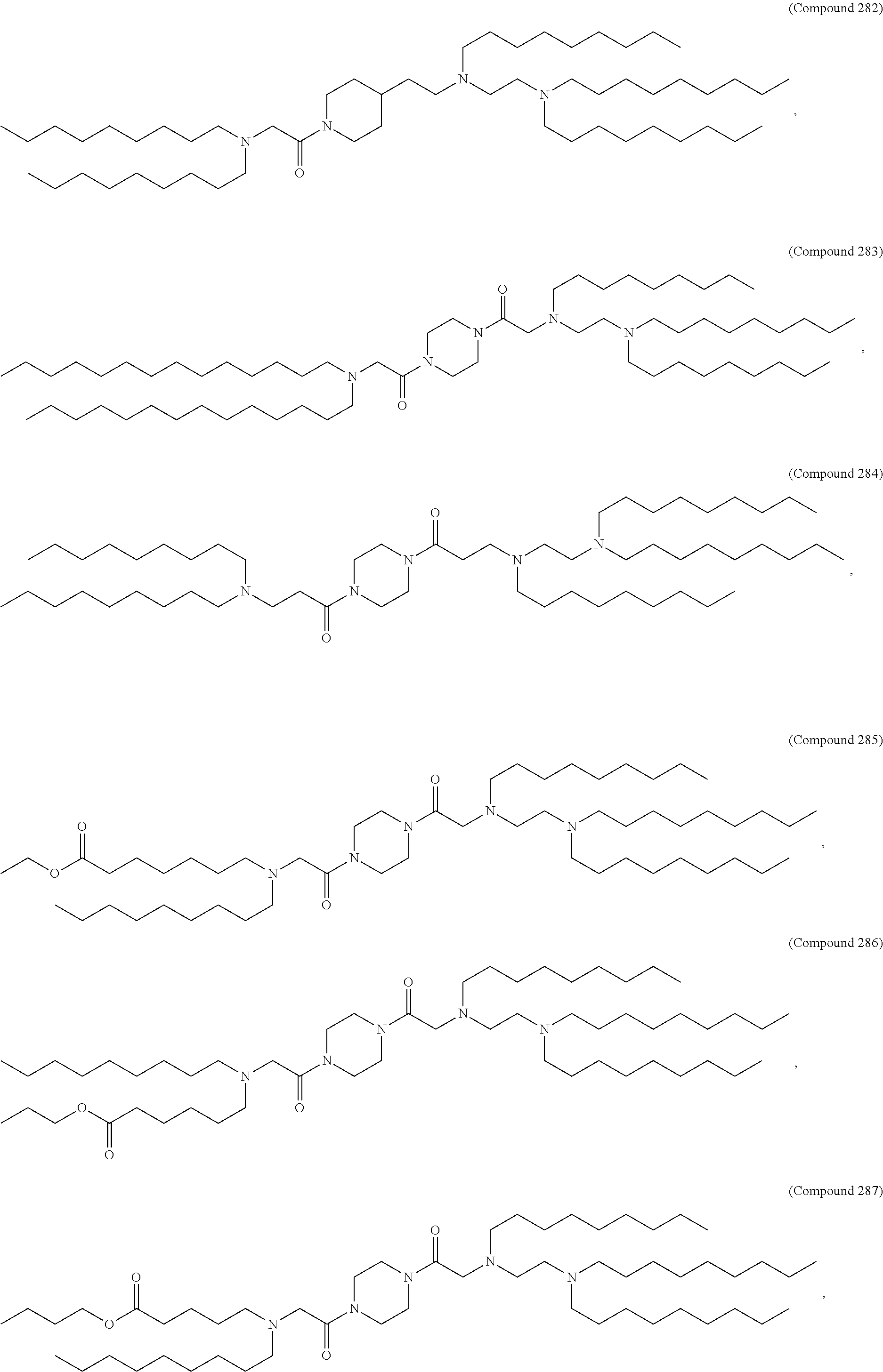

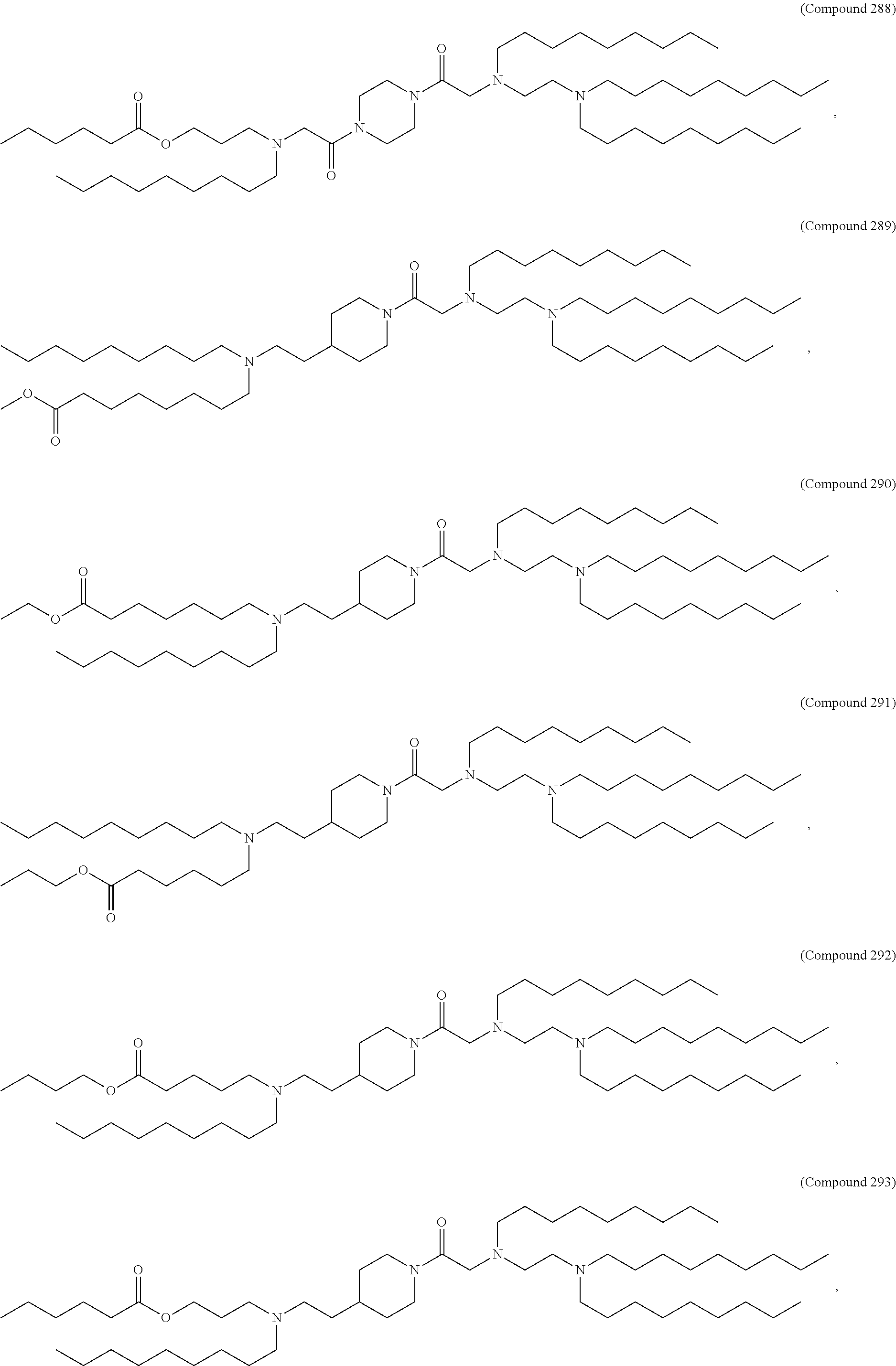

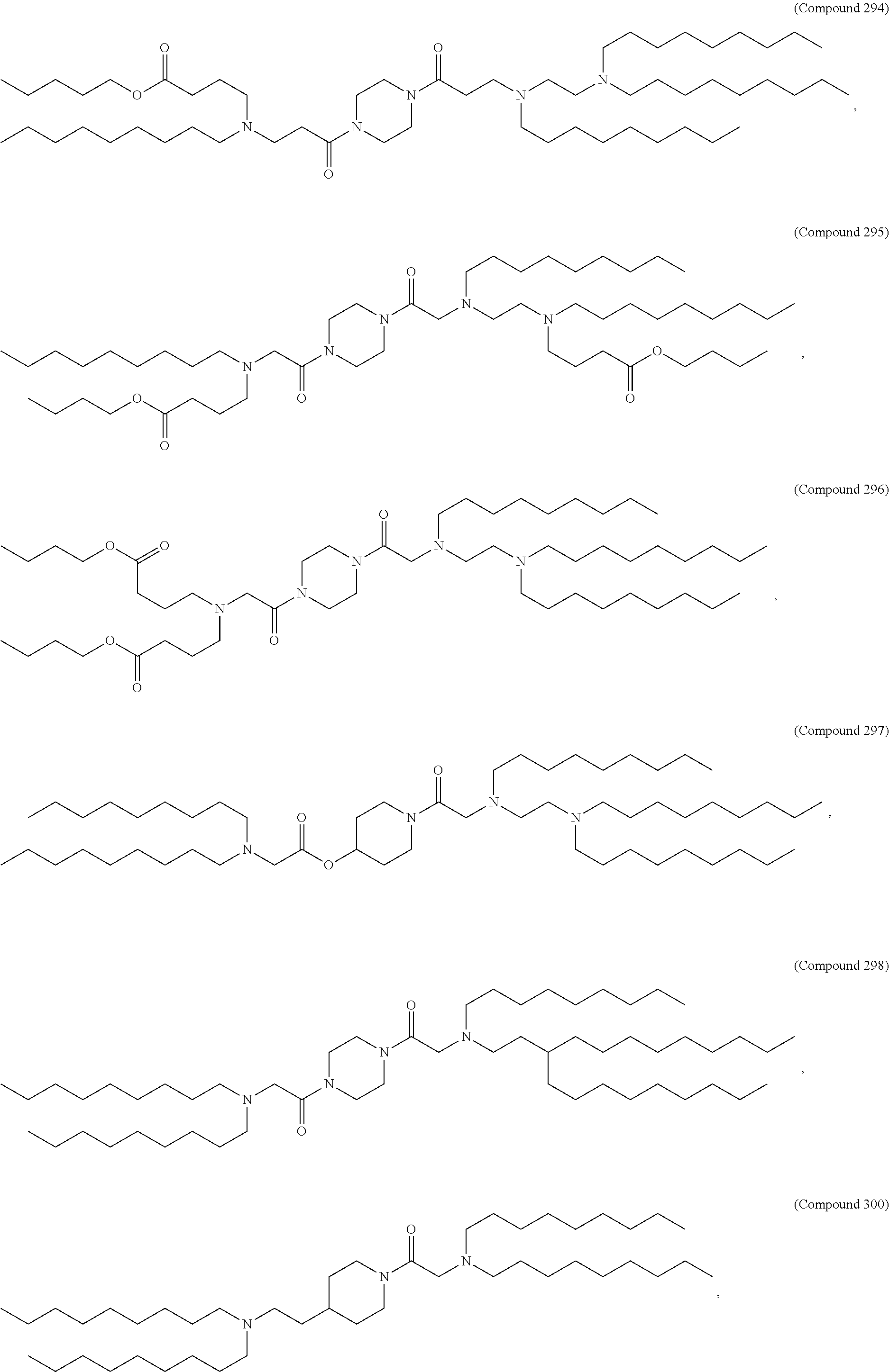

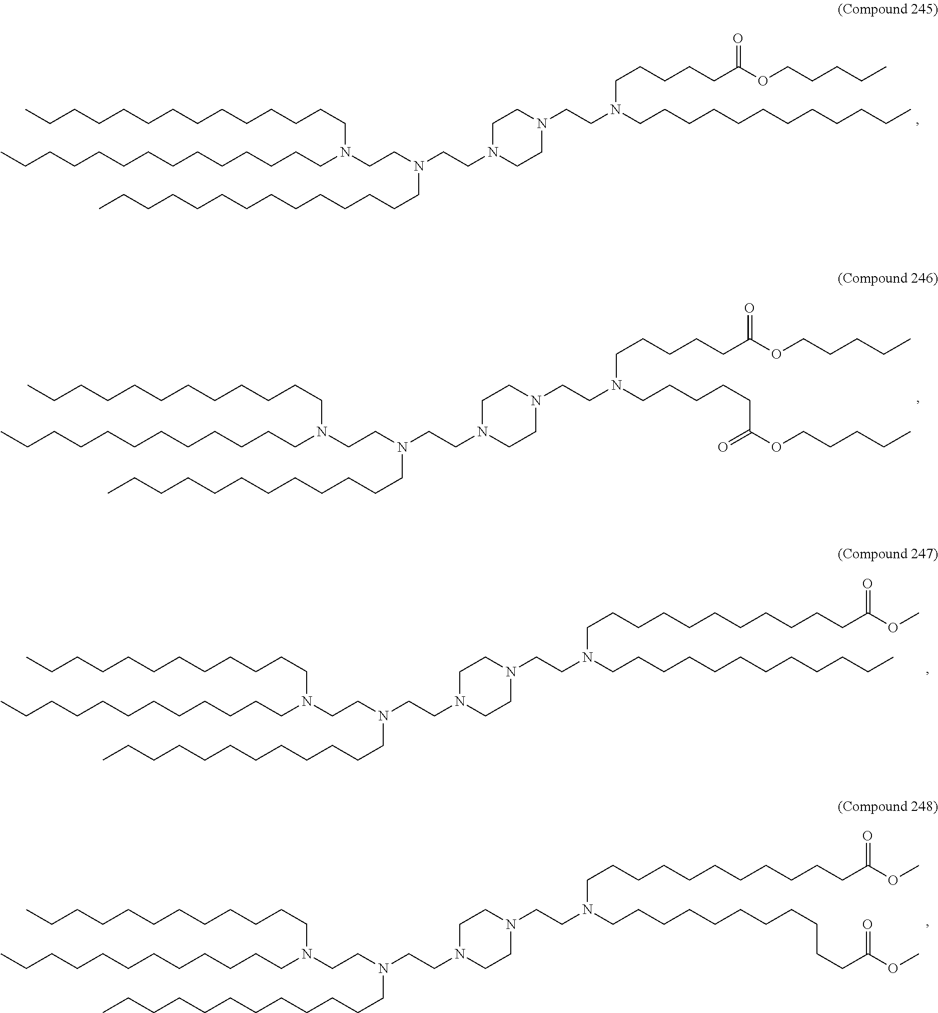

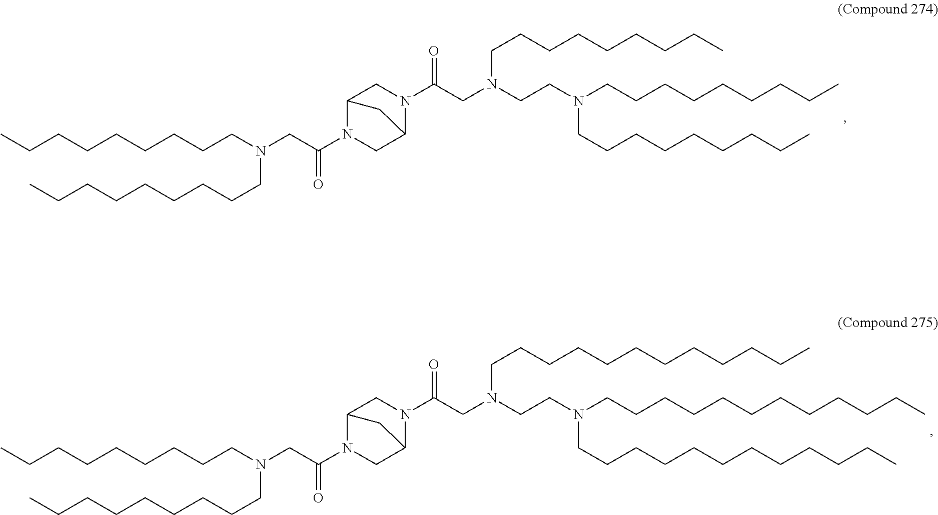

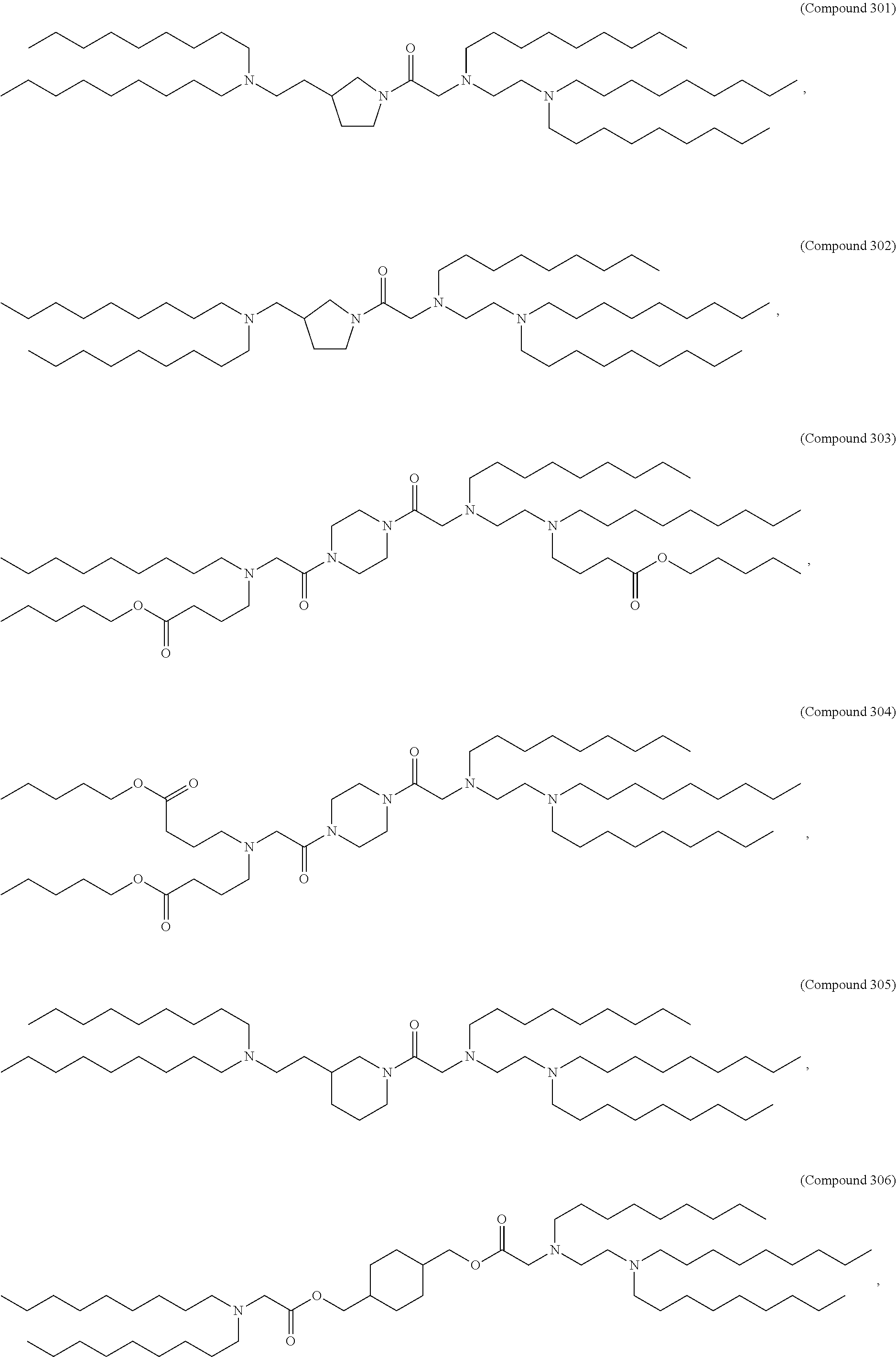

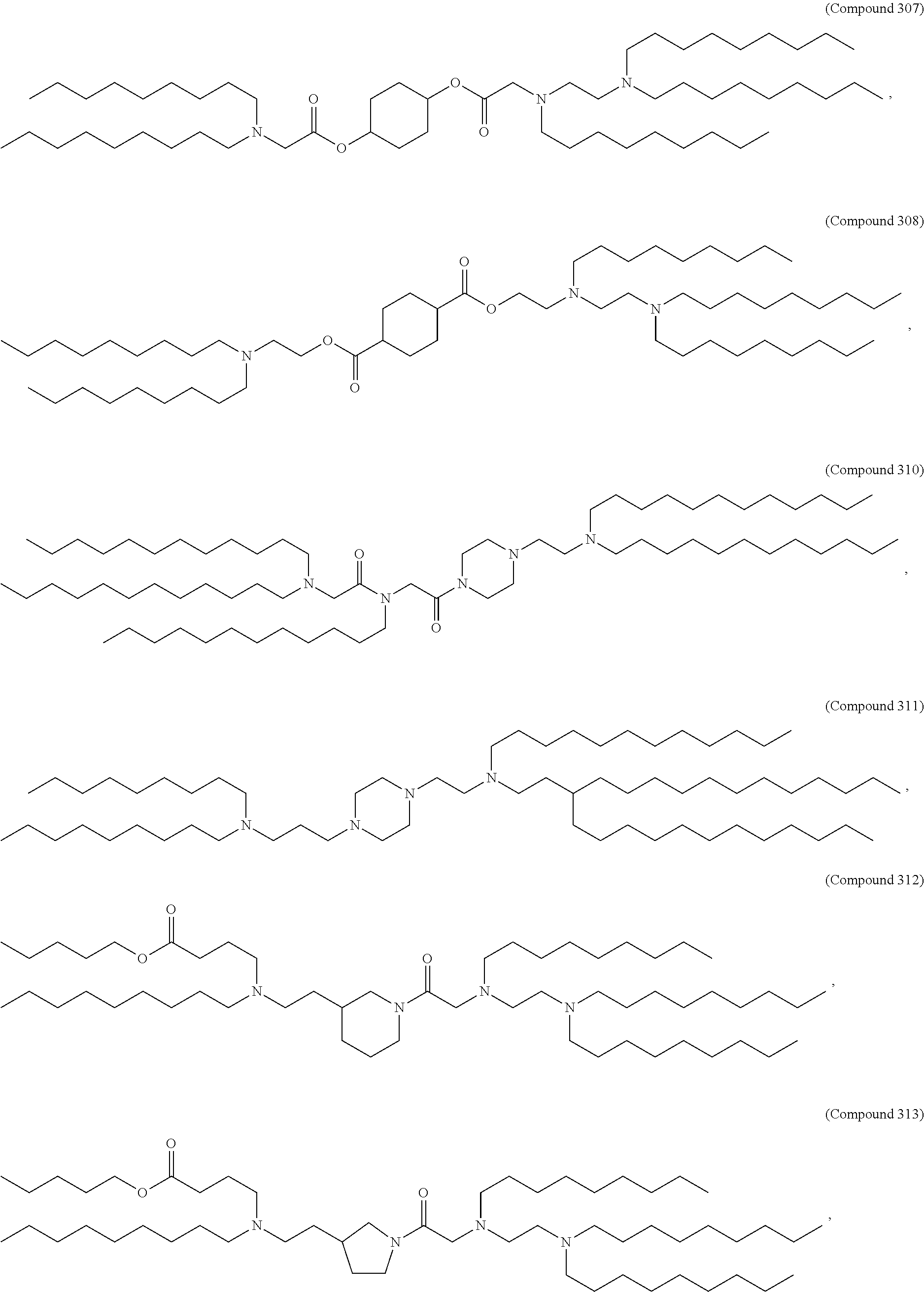

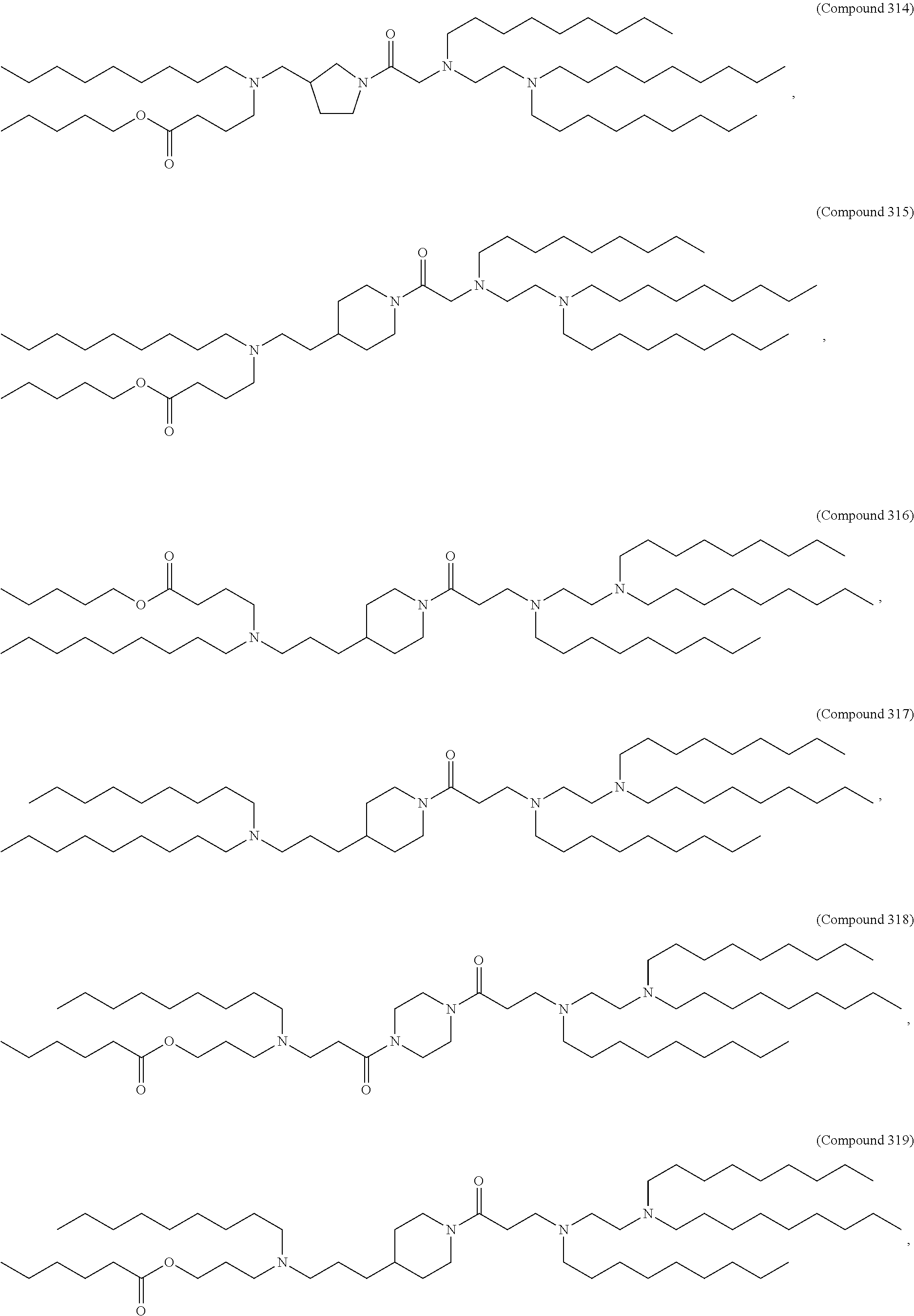

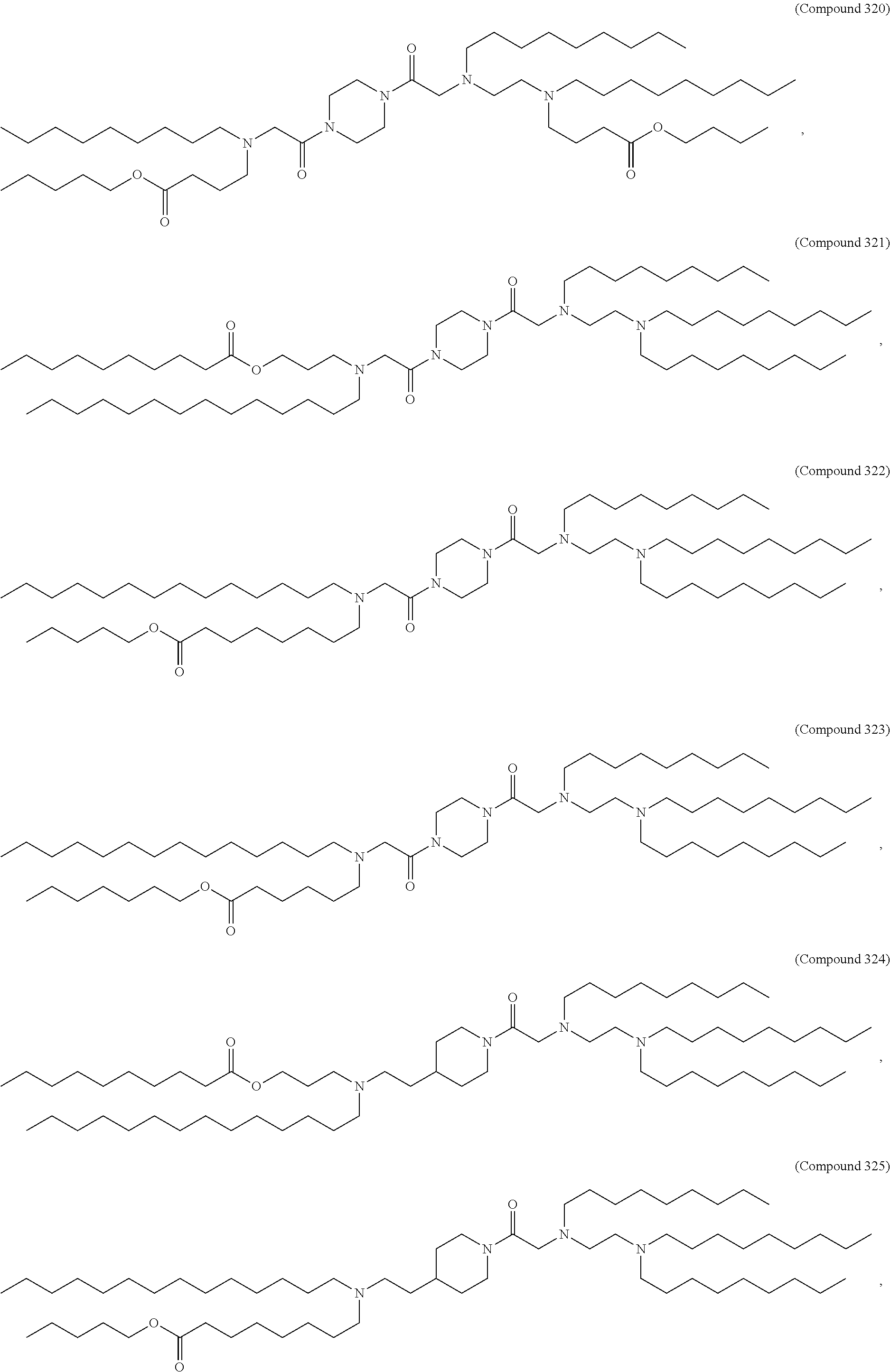

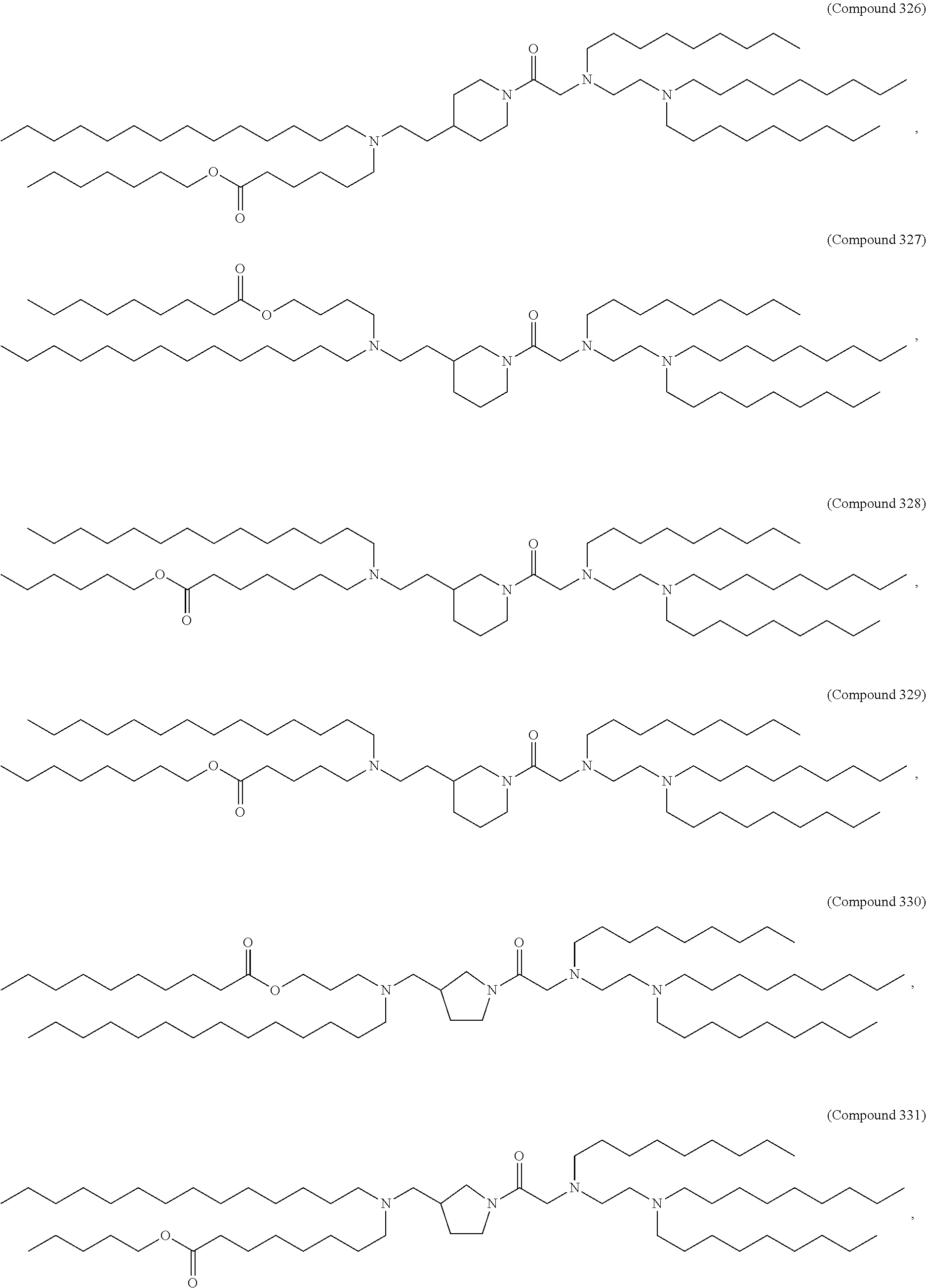

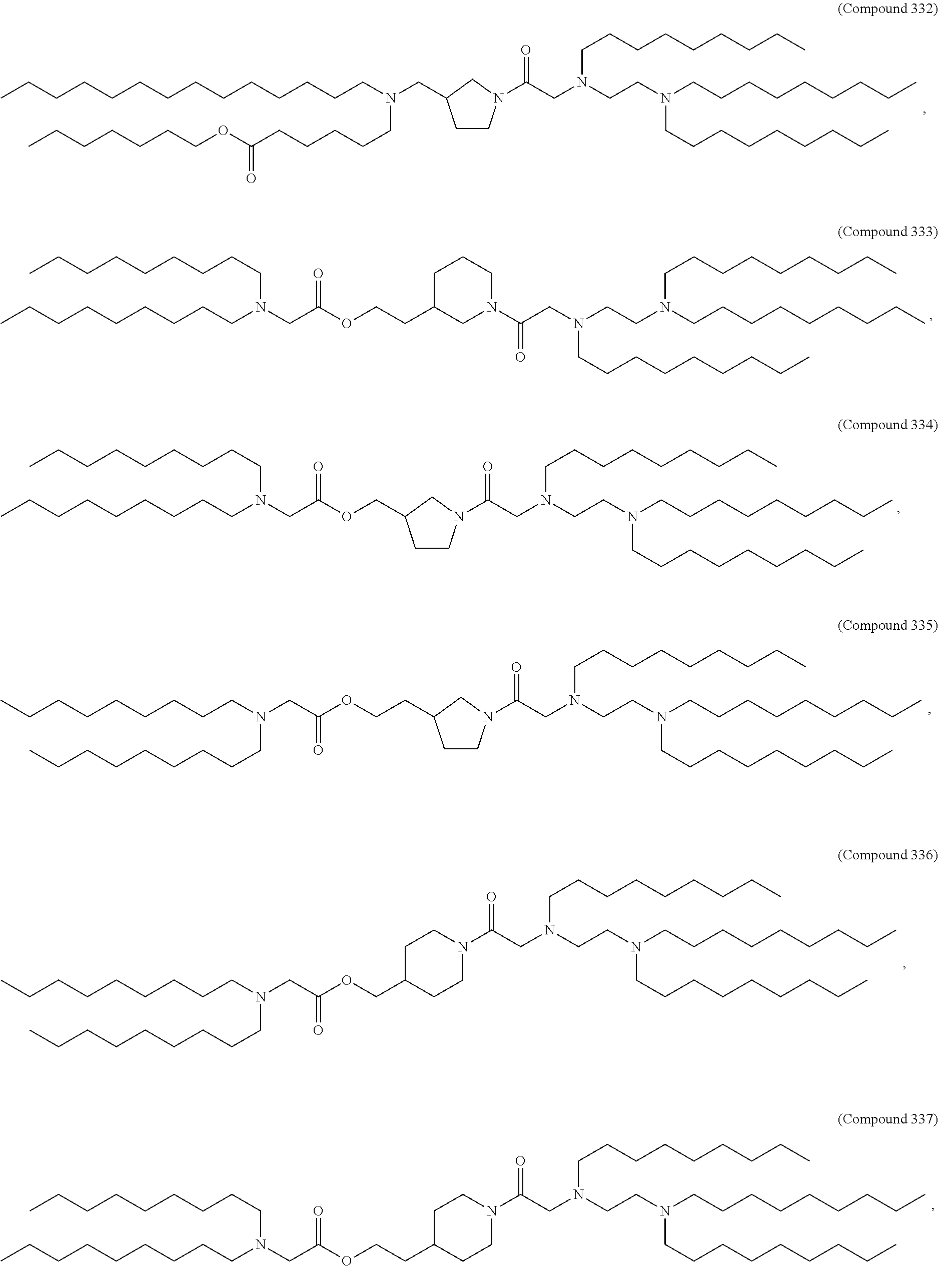

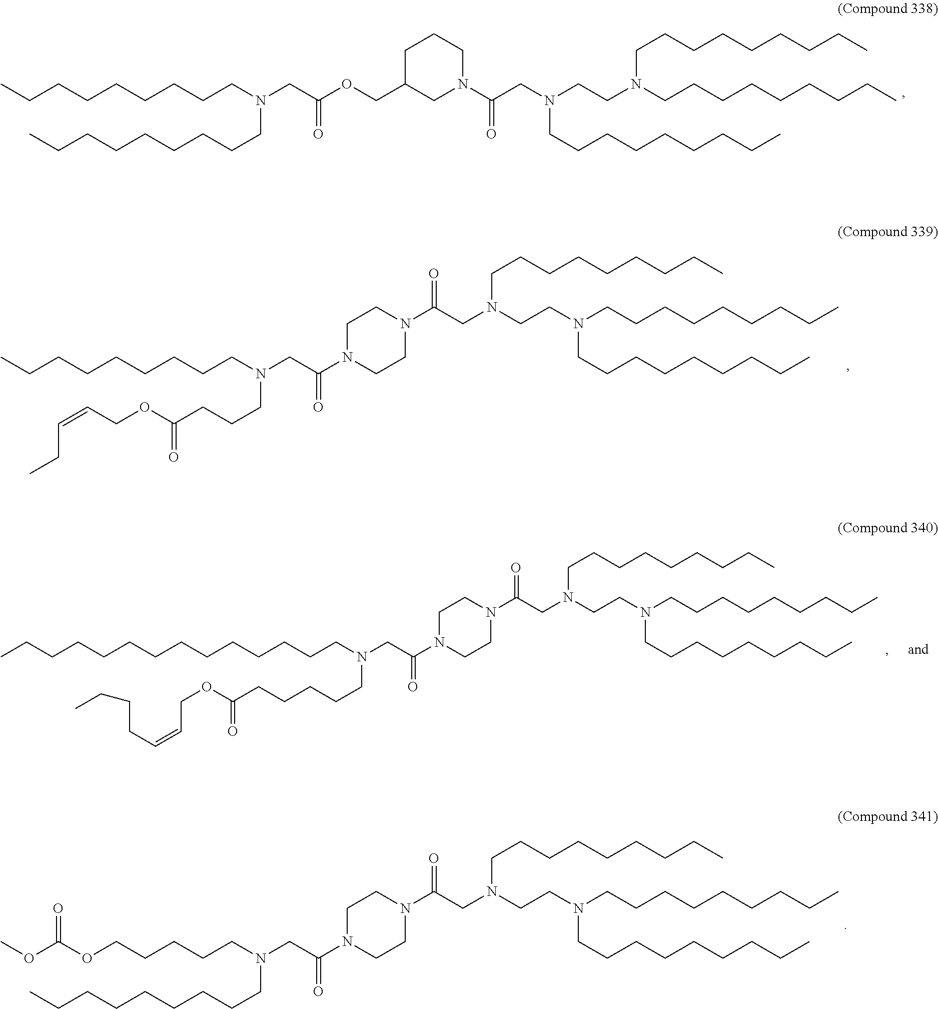

In some embodiments, the delivery agent comprises Compound 236.

In some embodiments, the delivery agent comprises a compound having the formula (IV)

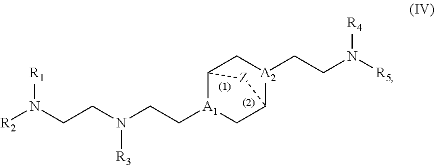

or salts or stereoisomer thereof, wherein
A₁ and A₂ are each independently selected from CH or N and at least one of A₁ and A₂ is N;
Z is CH₂ or absent wherein when Z is CH₂, the dashed lines (1) and (2) each represent a single bond; and when Z is absent, the dashed lines (1) and (2) are both absent;
R₁, R₂, R₃, R₄, and R₅ are independently selected from the group consisting of C₆₋₂₀ alkyl and C₆₋₂₀ alkenyl;
wherein when ring A is then

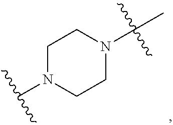 [SMILES: *N1CCN(*)CC1]

i) R₁, R₂, R₃, R₄, and R₅ are the same, wherein R₁ is not C₁₂ alkyl, C₁₈ alkyl, or C₁₈ alkenyl;
ii) only one of R₁, R₂, R₃, R₄, and R₅ is selected from C₆₋₂₀ alkenyl;
iii) at least one of R₁, R₂, R₃, R₄, and R₅ have a different number of carbon atoms than at least one other of R₁, R₂, R₃, R₄, and R₅;
iv) R₁, R₂, and R₃ are selected from C₆₋₂₀ alkenyl, and R₄ and R₅ are selected from C₆₋₂₀ alkyl; or
v) R₁, R₂, and R₃ are selected from C₆₋₂₀ alkyl, and R₄ and R₅ are selected from C₆₋₂₀ alkenyl.

In some embodiments, the compound is of formula (IVa):

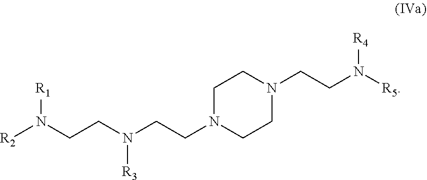

The compounds of Formula (IV) or (IVa) include one or more of the following features when applicable.

In some embodiments, Z is CH₂.

In some embodiments, Z is absent.

In some embodiments, at least one of A₁ and A₂ is N.

In some embodiments, each of A₁ and A₂ is N.

In some embodiments, each of A₁ and A₂ is CH.

In some embodiments, A₁ is N and A₂ is CH.

In some embodiments, A₁ is CH and A₂ is N.

In some embodiments, R₁, R₂, R₃, R₄, and R₅ are the same, and are not C₁₂ alkyl, C₁₈ alkyl, or C₁₈ alkenyl. In some embodiments, R₁, R₂, R₃, R₄, and R₅ are the same and are C₉ alkyl or C₁₄ alkyl.

In some embodiments, only one of R₁, R₂, R₃, R₄, and R₅ is selected from C₆₋₂₀ alkenyl. In certain such embodiments, R₁, R₂, R₃, R₄, and R₅ have the same number of carbon atoms. In some embodiments, R₄ is selected from C₅₋₂₀ alkenyl. For example, R₄ may be C₁₂ alkenyl or C₁₈ alkenyl.

In some embodiments, at least one of R₁, R₂, R₃, R₄, and R₅ have a different number of carbon atoms than at least one other of R₁, R₂, R₃, R₄, and R₅.

In certain embodiments, R₁, R₂, and R₃ are selected from C₆₋₂₀ alkenyl, and R₄ and R₅ are selected from C₆₋₂₀ alkyl. In other embodiments, R₁, R₂, and R₃ are selected from C₆₋₂₀ alkyl, and R₄ and R₅ are selected from C₆₋₂₀ alkenyl. In some embodiments, R₁, R₂, and R₃ have the same number of carbon atoms, and/or R₄ and R₅ have the same number of carbon atoms. For example, R₁, R₂, and R₃, or R₄ and R₅, may have 6, 8, 9, 12, 14, or 18 carbon atoms. In some embodiments, R₁, R₂, and R₃, or R₄ and R₅, are C₁₈ alkenyl (e.g., linoleyl). In some embodiments, R₁, R₂, and R₃, or R₄ and R₅, are alkyl groups including 6, 8, 9, 12, or 14 carbon atoms.

In some embodiments, R₁ has a different number of carbon atoms than R₂, R₃, R₄, and R₅. In other embodiments, R₃ has a different number of carbon atoms than R₁, R₂, R₄, and R₅. In further embodiments, R₄ has a different number of carbon atoms than R₁, R₂, R₃, and R₅.

In some embodiments, the compound is selected from the group consisting of:

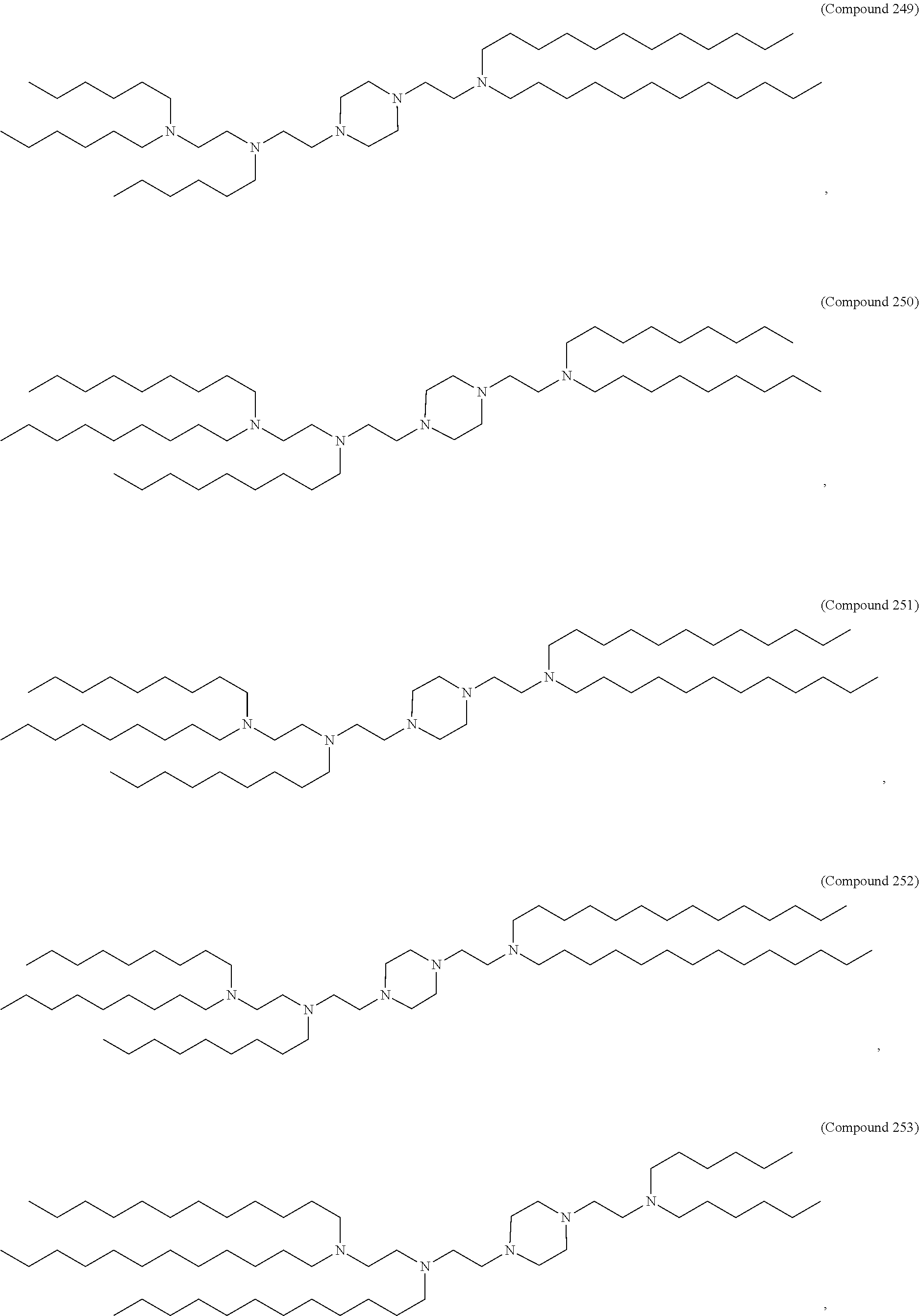

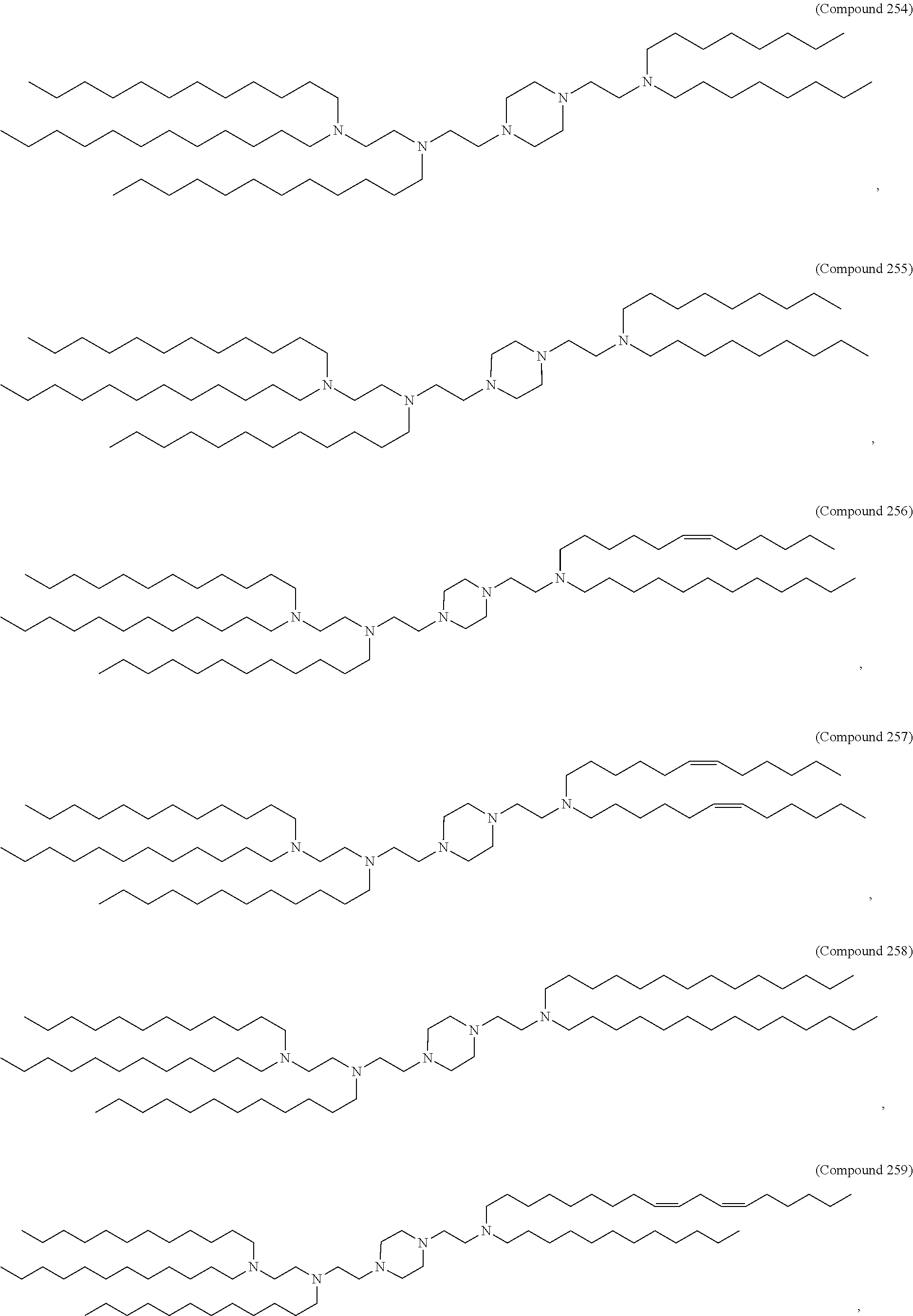

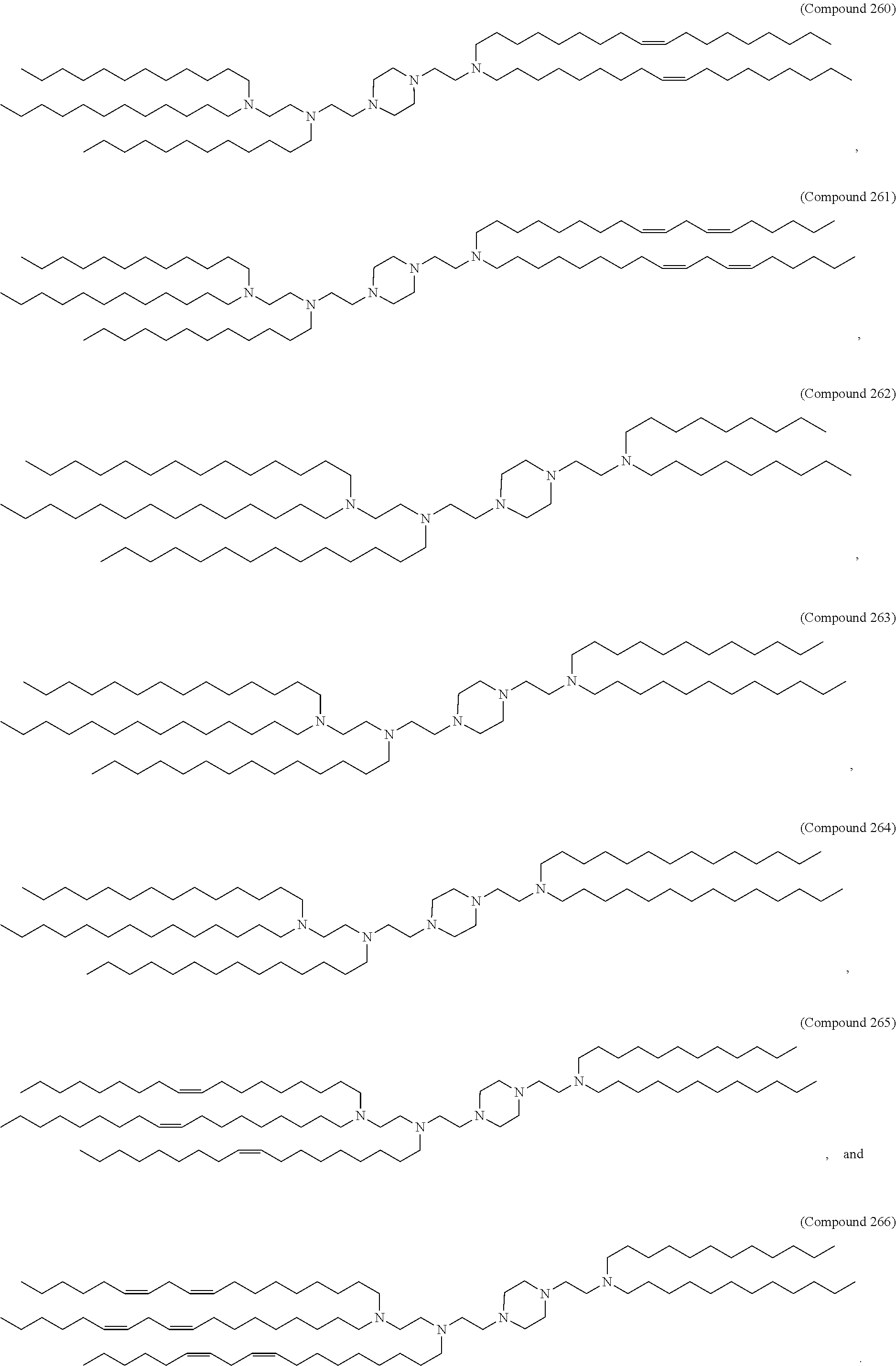

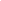

In other embodiments, the delivery agent comprises a compound having the formula (V)

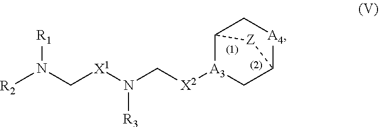

or salts or stereoisomers thereof, in which
A₃ is CH or N;
A₄ is CH₂ or NH; and at least one of A₃ and A₄ is N or NH;
Z is CH₂ or absent wherein when Z is CH₂, the dashed lines (1) and (2) each represent a single bond; and when Z is absent, the dashed lines (1) and (2) are both absent;
R₁, R₂, and R₃ are independently selected from the group consisting of C₅₋₂₀ alkyl, C₅₋₂₀ alkenyl, —R″MR′, —R*YR″, —YR″, and —R*OR″;
each M is independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
X¹ and X² are independently selected from the group consisting of —CH₂—, (CH₂) 2-, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂-12 alkenyl, and H; and
each R″ is independently selected from the group consisting of C₃₋₁₂ alkyl and C₃₋₁₂ alkenyl.

In some embodiments, the compound is of formula (Va):

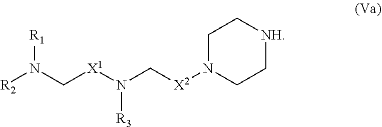

The compounds of Formula (V) or (Va) include one or more of the following features when applicable.

In some embodiments, Z is CH₂.

In some embodiments, Z is absent.

In some embodiments, at least one of A₃ and A₄ is N or NH.

In some embodiments, A₃ is N and A₄ is NH.

In some embodiments, A₃ is N and A₄ is CH₂.

In some embodiments, A₃ is CH and A₄ is NH.

In some embodiments, at least one of X¹ and X² is not —CH₂—. For example, in certain embodiments, X¹ is not —CH₂—. In some embodiments, at least one of X¹ and X² is —C(O)—.

In some embodiments, X² is —C(O)—, —C(O)O—, —OC(O)—, —C(O)—CH₂—, —CH₂—C(O)—, —C(O)O—CH₂—, —OC(O)—CH₂—, —CH₂—C(O)O—, or —CH₂—OC(O)—.

In some embodiments, R₁, R₂, and R₃ are independently selected from the group consisting of C₅₋₂₀ alkyl and C₅₋₂₀ alkenyl. In some embodiments, R₁, R₂, and R₃ are the same. In certain embodiments, R₁, R₂, and R₃ are C₆, C₉, C₁₂, or C₁₄ alkyl. In other embodiments, R₁, R₂, and R₃ are C₁₈ alkenyl. For example, R₁, R₂, and R₃ may be linoleyl.

In some embodiments, the compound is selected from the group consisting of:

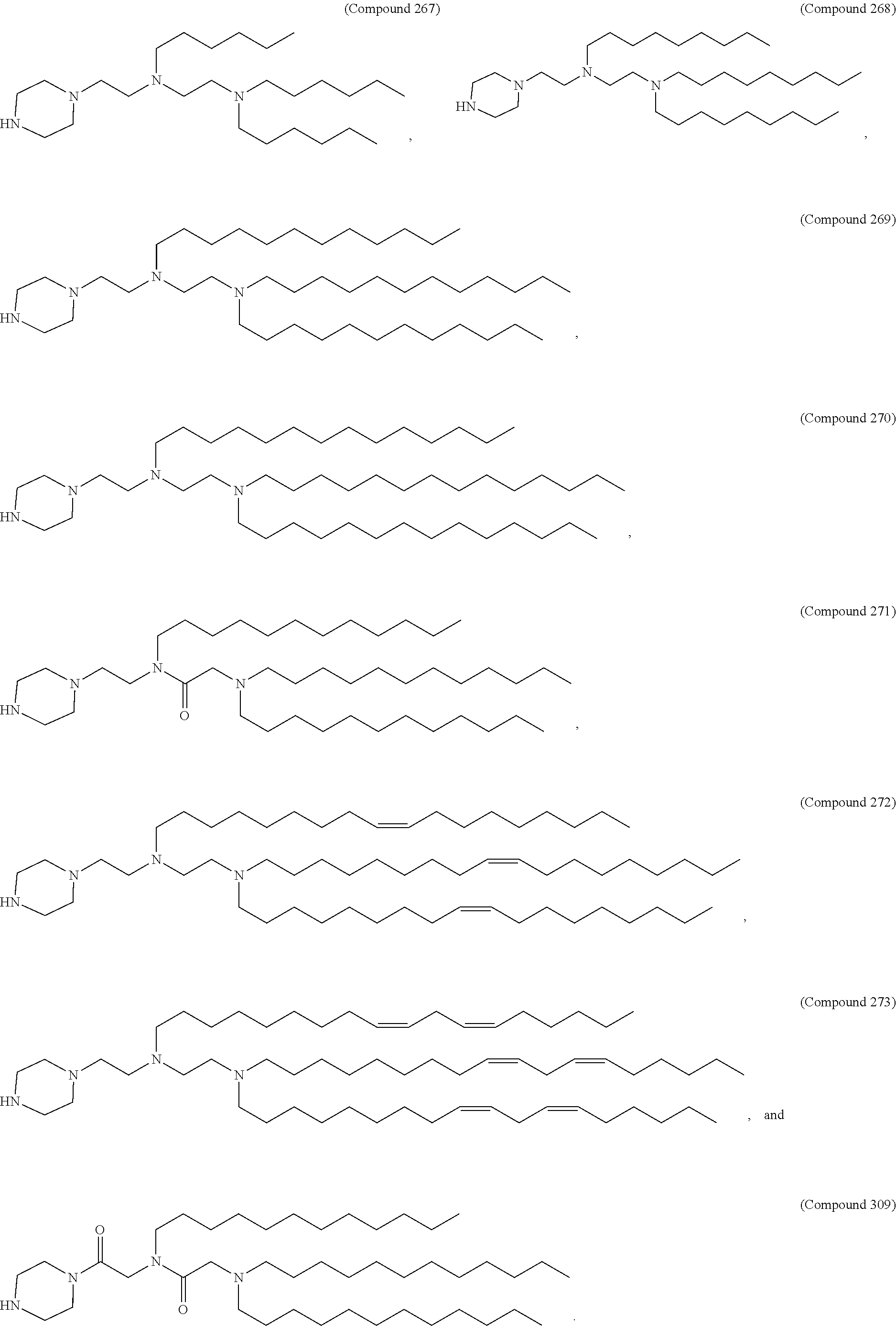

In other embodiments, the delivery agent comprises a compound having the formula (VI):

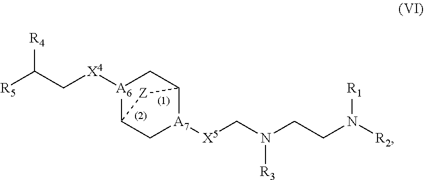

or salts or stereoisomers thereof, in which
A₆ and A₇ are each independently selected from CH or N, wherein at least one of A₆ and A₇ is N;
Z is CH₂ or absent wherein when Z is CH₂, the dashed lines (1) and (2) each represent a single bond; and when Z is absent, the dashed lines (1) and (2) are both absent;
X⁴ and X⁵ are independently selected from the group consisting of —CH₂—, —CH₂) 2-, —CHR—, —CHY—, —C(O)—, —C(O)O—, —OC(O)—, —C(O)—CH₂—, —CH₂—C(O)—, —C(O)O—CH₂—, —OC(O)—CH₂—, —CH₂—C(O)O—, —CH₂—OC(O)—, —CH(OH)—, —C(S)—, and —CH(SH)—;
R₁, R₂, R₃, R₄, and R₅ each are independently selected from the group consisting of C₅₋₂₀ alkyl, C₅₋₂₀ alkenyl, —R″MR′, —R*YR″, —YR″, and —R*OR″;
each M is independently selected from the group consisting of —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂— an aryl group, and a heteroaryl group;
each Y is independently a C₃₋₆ carbocycle;
each R* is independently selected from the group consisting of C₁₋₁₂ alkyl and C₂₋₁₂ alkenyl;
each R is independently selected from the group consisting of C₁₋₃ alkyl and a C₃₋₆ carbocycle;
each R′ is independently selected from the group consisting of C₁₋₁₂ alkyl, C₂-12 alkenyl, and H; and
each R″ is independently selected from the group consisting of C₃₋₁₂ alkyl and C₃₋₁₂ alkenyl.

In some embodiments, R₁, R₂, R₃, R₄, and R₅ each are independently selected from the group consisting of C₆₋₂₀ alkyl and C₆₋₂₀ alkenyl.

In some embodiments, R₁ and R₂ are the same. In certain embodiments, R₁, R₂, and R₃ are the same. In some embodiments, R₄ and R₅ are the same. In certain embodiments, R₁, R₂, R₃, R₄, and R₅ are the same.

In some embodiments, at least one of R₁, R₂, R₃, R₄, and R₅ is C₉₋₁₂ alkyl. In certain embodiments, each of R₁, R₂, R₃, R₄, and R₅ independently is C₉, C₁₂ or C₁₄ alkyl. In certain embodiments, each of R₁, R₂, R₃, R₄, and R₅ is C₉ alkyl.

In some embodiments, A₆ is N and A₇ is N. In some embodiments, A₆ is CH and A₇ is N.

In some embodiments, X⁴ is —CH₂— and X⁵ is —C(O)—. In some embodiments, X⁴ and X⁵ are —C(O)—.

In some embodiments, when A₆ is N and A₇ is N, at least one of X⁴ and X⁵ is not-CH₂—, e.g., at least one of X⁴ and X⁵ is —C(O)—. In some embodiments, when A₆ is N and A₇ is N, at least one of R₁, R₂, R₃, R₄, and R₅ is —R″MR′.

In some embodiments, at least one of R₁, R₂, R₃, R₄, and R₅ is not —R″MR′.

In some embodiments, the compound is

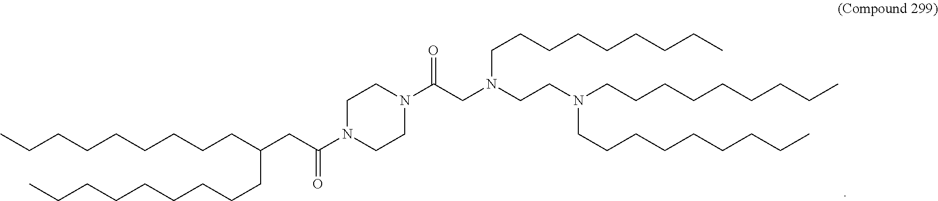 [SMILES: CCCCCCCCCC(CCCCCCCCC)CC(=O)N1CCN(C(=O)CN(CCCCCCCCC)CCN(CCCCCCCCC)CCCCCCCCC)CC1]

In other embodiments, the delivery agent comprises a compound having the formula:

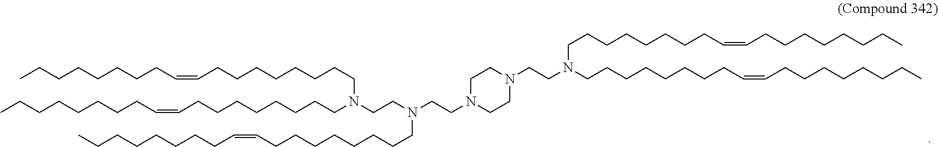 [SMILES: CCCCCCCC/C=C\CCCCCCCCN(CCCCCCCC/C=C\CCCCCCCC)CCN(CCCCCCCC/C=C\CCCCCCCC)CCN1CCN(CCN(CCCCCCCC/C=C\CCCCCCCC)CCCCCCCC/C=C\CCCCCCCC)CC1]

Amine moieties of the lipid compounds disclosed herein can be protonated under certain conditions. For example, the central amine moiety of a lipid according to formula (I) is typically protonated (i.e., positively charged) at a pH below the pKa of the amino moiety and is substantially not charged at a pH above the pKa. Such lipids can be referred to ionizable amino lipids.

In one specific embodiment, the ionizable amino lipid is Compound 18. In another embodiment, the ionizable amino lipid is Compound 236.

In some embodiments, the amount the ionizable amino lipid, e.g., compound of formula (I) ranges from about 1 mol % to 99 mol % in the lipid composition.

In one embodiment, the amount of the ionizable amino lipid, e.g., compound of formula (I) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the ionizable amino lipid, e.g., the compound of formula (I) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the ionizable amino lipid, e.g., compound of formula (I) is about 50 mol % in the lipid composition.

In addition to the ionizable amino lipid disclosed herein, e.g., compound of formula (I), the lipid composition of the pharmaceutical compositions disclosed herein can comprise additional components such as phospholipids, structural lipids, PEG-lipids, and any combination thereof.

b. Phospholipids

The lipid composition of the pharmaceutical composition disclosed herein can comprise one or more phospholipids, for example, one or more saturated or (poly) unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

Examples of phospholipids include, but are not limited to, the following:

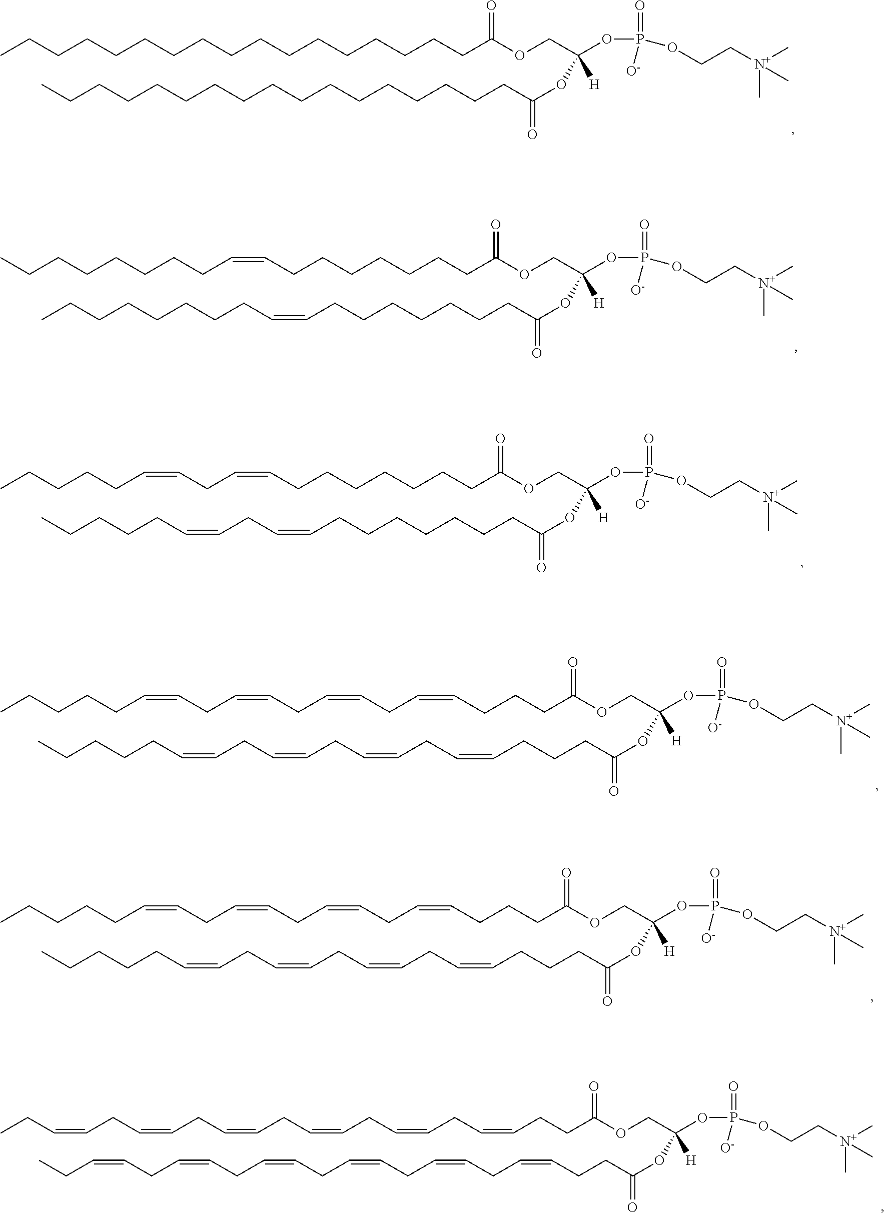

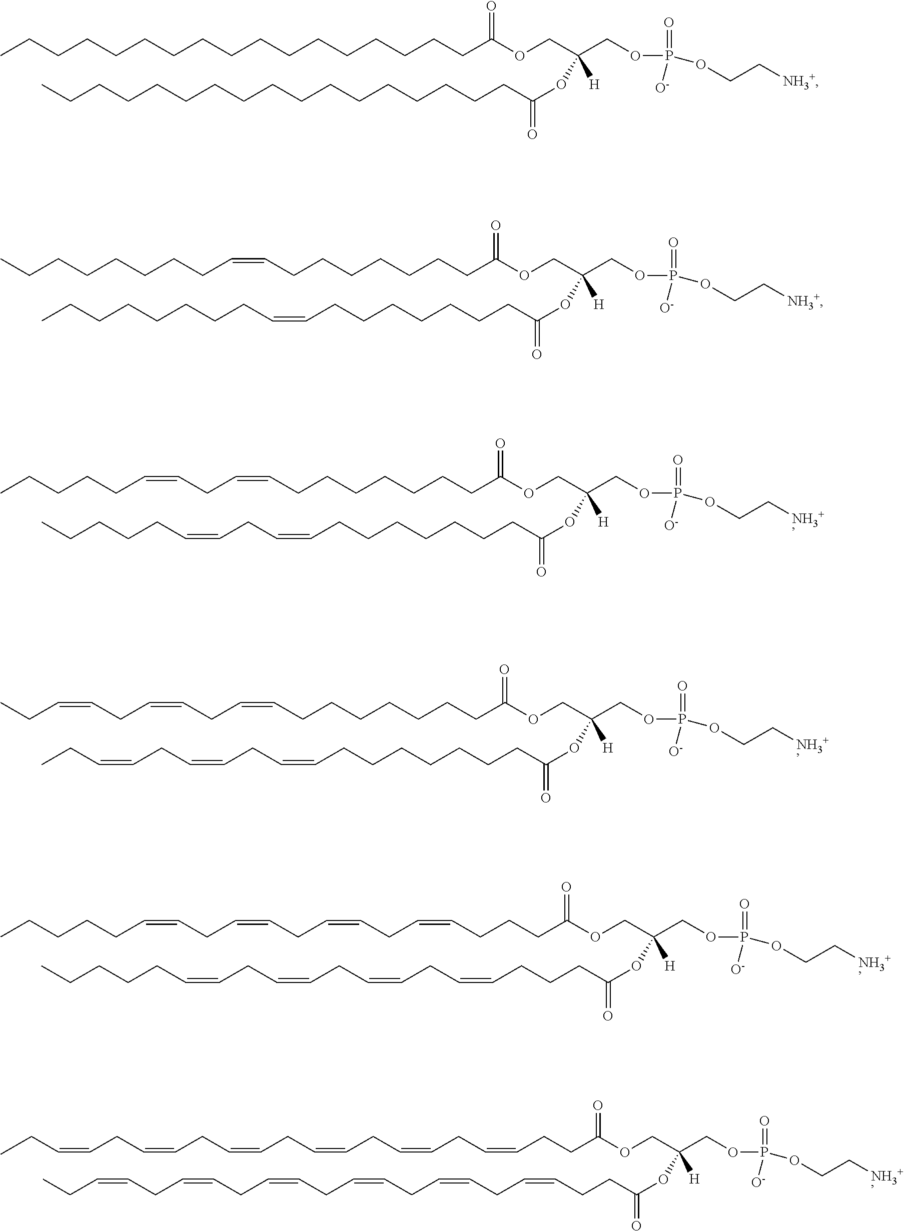

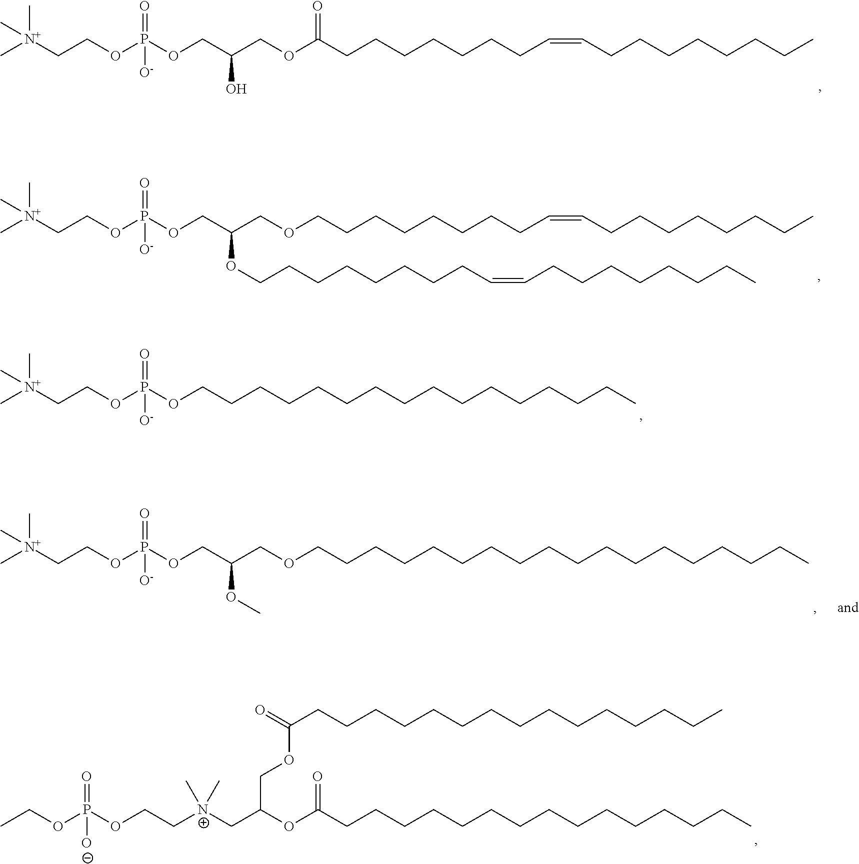

In certain embodiments, a phospholipid useful or potentially useful in the present invention is an analog or variant of DSPC (1,2-dioctadecanoyl-sn-glycero-3-phosphocholine). In certain embodiments, a phospholipid useful or potentially useful in the present invention is a compound of Formula (IX):

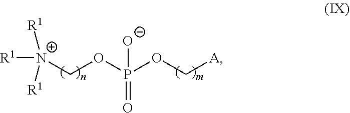

(or a salt thereof, wherein:
each R₁ is independently optionally substituted alkyl; or optionally two R₁ are joined together with the intervening atoms to form optionally substituted monocyclic carbocyclyl or optionally substituted monocyclic heterocyclyl; or optionally three R₁ are joined together with the intervening atoms to form optionally substituted bicyclic carbocyclyl or optionally substitute bicyclic heterocyclyl;
n is 1, 2, 3, 4, 5, 6, 7, 8, 9, or 10;
m is 0, 1, 2, 3, 4, 5, 6, 7, 8, 9, or 10;
A is of the formula:

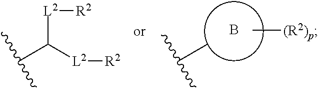

each instance of L2 is independently a bond or optionally substituted C₁₋₆ alkylene, wherein one methylene unit of the optionally substituted C₁₋₆ alkylene is optionally replaced with —O—, —N(RN)—, —S—, —C(O)—, —C(O)N(R^{N})—, —NR^{N}C(O)—, —C(O)O—, —OC(O)—, —OC(O)O—, —OC(O)N(R^{N})—, —NR^{N}C(O)O—, or —NR^{N}C(O)N(R^{N})—;
each instance of R₂ is independently optionally substituted C₁₋₃₀ alkyl, optionally substituted C₁₋₃₀ alkenyl, or optionally substituted C₁₋₃₀ alkynyl; optionally wherein one or more methylene units of R₂ are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═N^{R}N)—, —C(═N^{R}N) N(R^{N})—, —NR^{N}C(═N^{R}N)—, —NR^{N}C(═N^{R}N) N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N}) S(O)—, —S(O)N(R^{N})—, —N(R^{N}) S(O)N(R^{N})—, —OS(O)N(R^{N})—, —N(R^{N}) S(O)O—, —S(O)₂—, —N(R^{N}) S(O)₂—, —S(O)₂N(R^{N})—, —N(R^{N}) S(O)₂N(R^{N})—, —OS(O)₂N(R^{N})—, or —N(R^{N}) S(O)₂O—;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2;
provided that the compound is not of the formula:

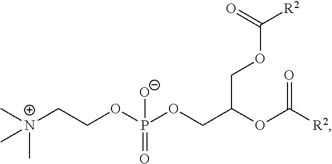

wherein each instance of R₂ is independently unsubstituted alkyl, unsubstituted alkenyl, or unsubstituted alkynyl.

### i) Phospholipid Head Modifications

In certain embodiments, a phospholipid useful or potentially useful in the present invention comprises a modified phospholipid head (e.g., a modified choline group). In certain embodiments, a phospholipid with a modified head is DSPC, or analog thereof, with a modified quaternary amine. For example, in embodiments of Formula (IX), at least one of R₁ is not methyl. In certain embodiments, at least one of R₁ is not hydrogen or methyl. In certain embodiments, the compound of Formula (IX) is of one of the following formulae:

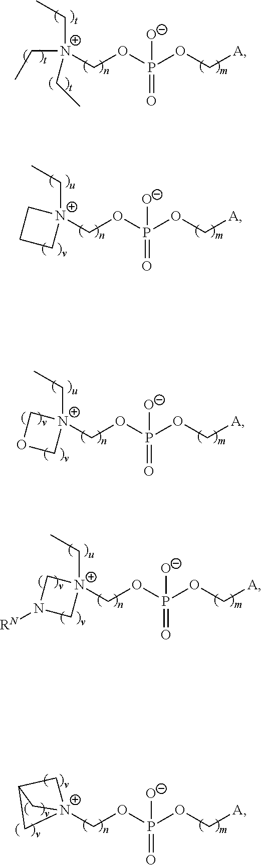

or a salt thereof, wherein:
each t is independently 1, 2, 3, 4, 5, 6, 7, 8, 9, or 10;
each u is independently 0, 1, 2, 3, 4, 5, 6, 7, 8, 9, or 10; and
each v is independently 1, 2, or 3.

In certain embodiments, the compound of Formula (IX) is of one of the following formulae:

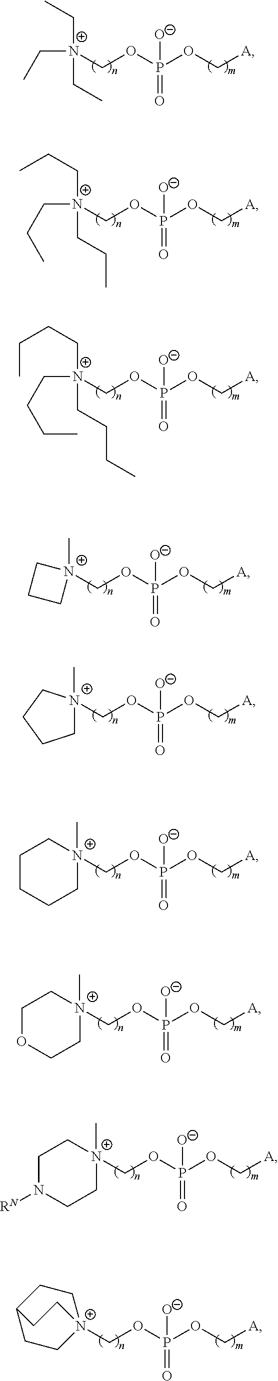

or a salt thereof.

In certain embodiments, a compound of Formula (IX) is one of the following:

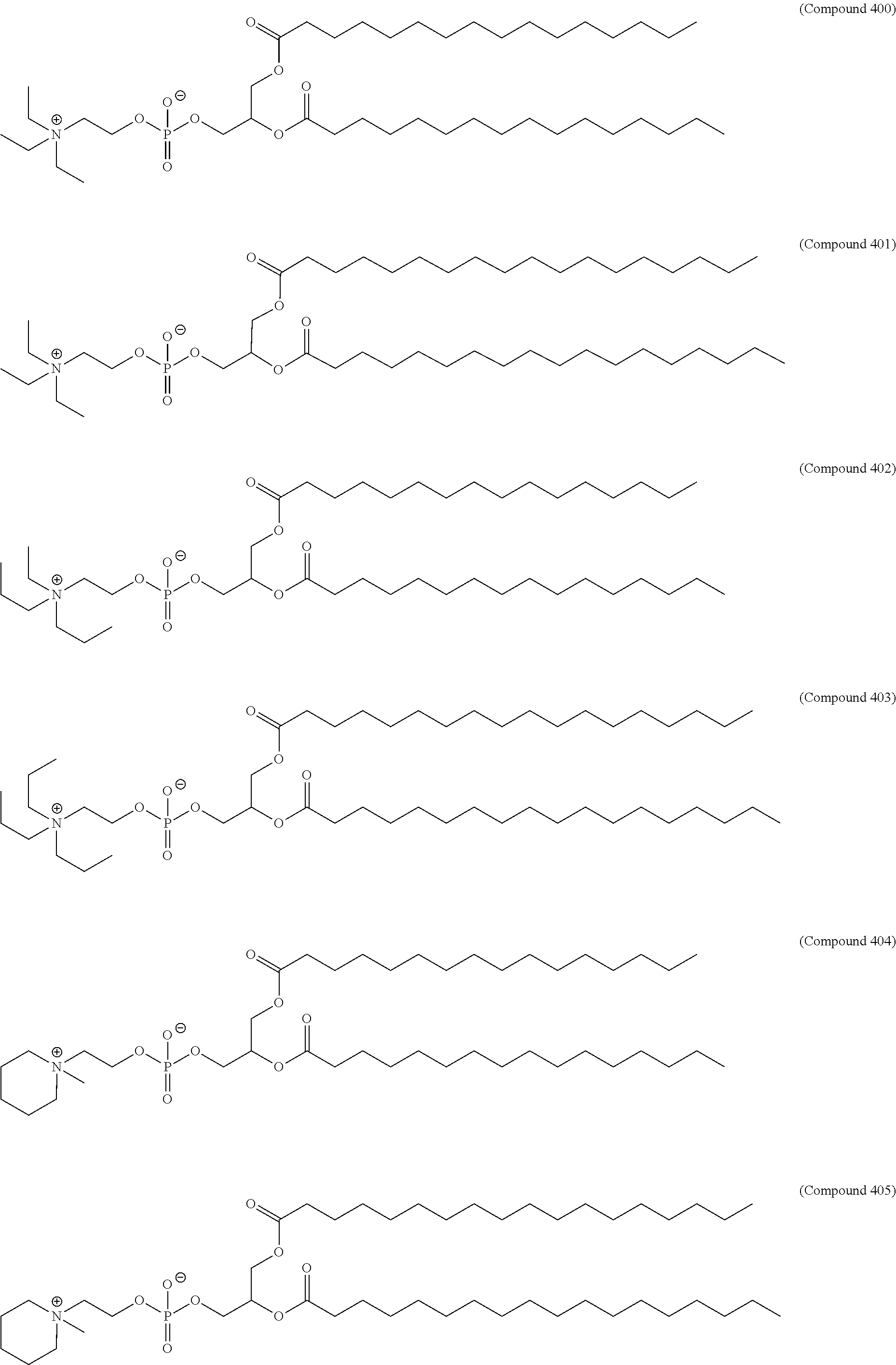

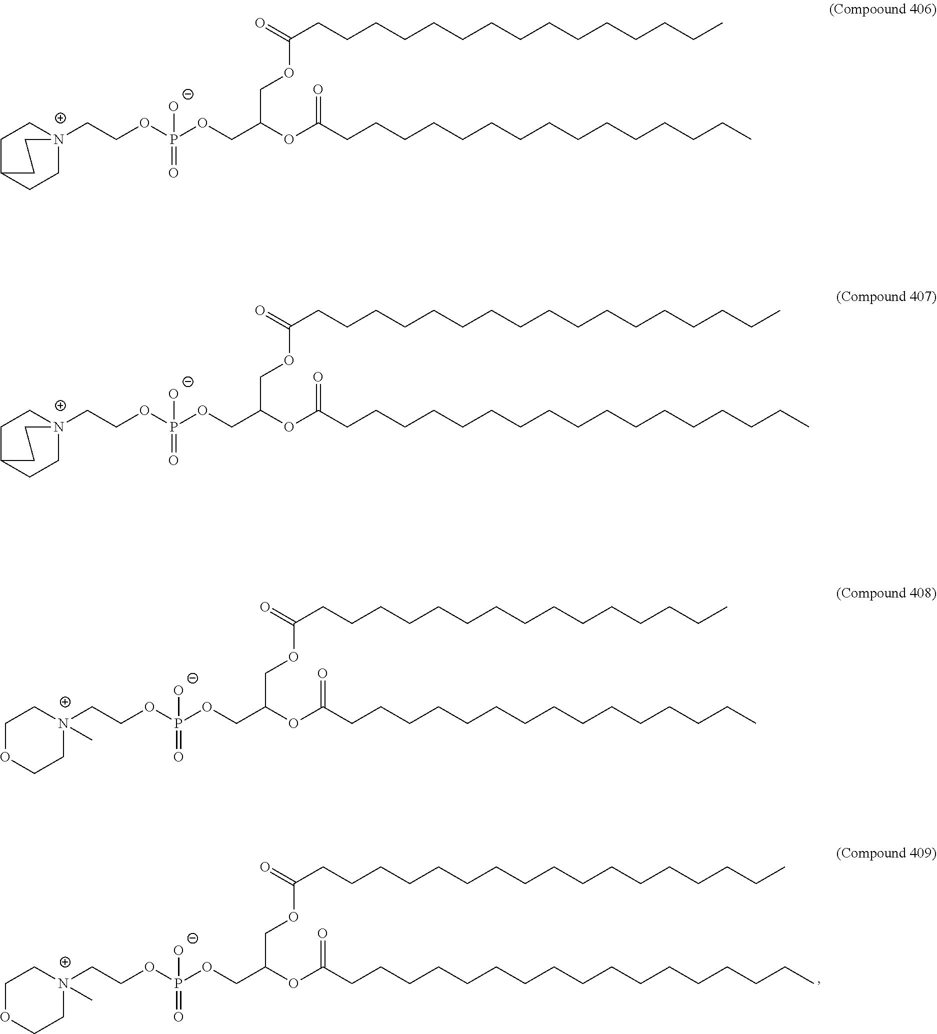 [SMILES: CCCCCCCCCCCCCCCC(=O)OCC(COP(=O)([O-])OCC[N+]12CCC(CC1)CC2)OC(=O)CCCCCCCCCCCCCCC ; CCCCCCCCCCCCCCCCCC(=O)OCC(COP(=O)([O-])OCC[N+]12CCC(CC1)CC2)OC(=O)CCCCCCCCCCCCCCCCC ; CCCCCCCCCCCCCCCC(=O)OCC(COP(=O)([O-])OCC[N+]1(C)CCOCC1)OC(=O)CCCCCCCCCCCCCCC ; CCCCCCCCCCCCCCCCCC(=O)OCC(COP(=O)([O-])OCC[N+]1(C)CCOCC1)OC(=O)CCCCCCCCCCCCCCCCC]

or a salt thereof.

In certain embodiments, a compound of Formula (IX) is of Formula (IX-a):

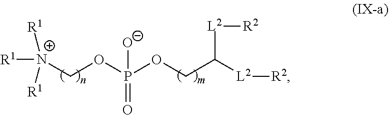

or a salt thereof.

In certain embodiments, phospholipids useful or potentially useful in the present invention comprise a modified core. In certain embodiments, a phospholipid with a modified core described herein is DSPC, or analog thereof, with a modified core structure. For example, in certain embodiments of Formula (IX-a), group A is not of the following formula:

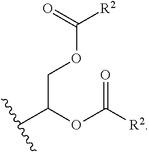

In certain embodiments, the compound of Formula (IX-a) is of one of the following formulae:

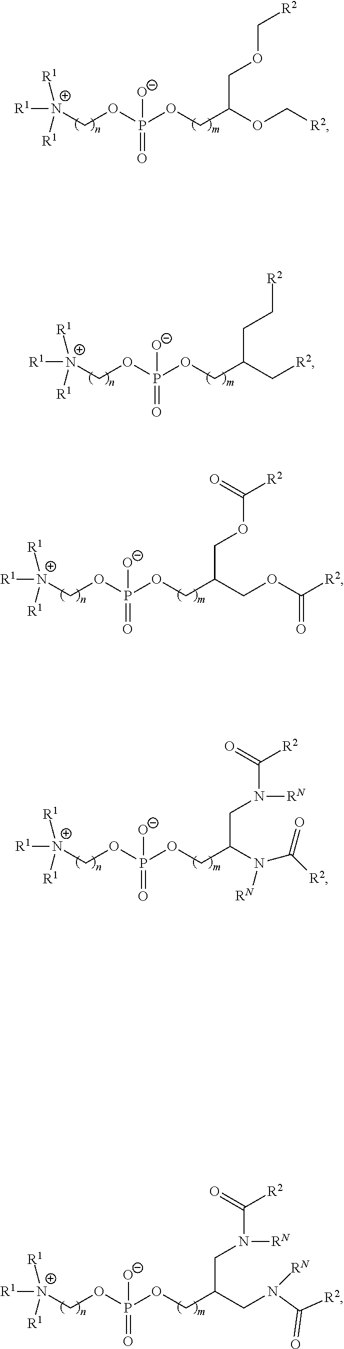

or a salt thereof.

In certain embodiments, a compound of Formula (IX) is one of the following:

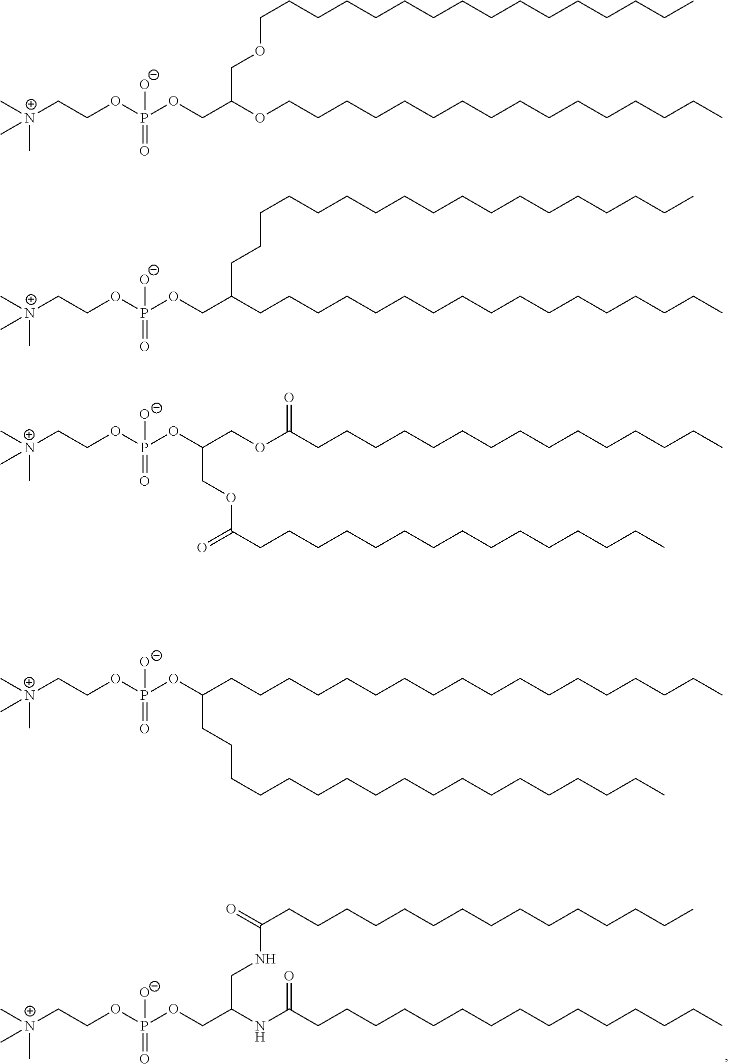

or salts thereof.

In certain embodiments, a phospholipid useful or potentially useful in the present invention comprises a cyclic moiety in place of the glyceride moiety. In certain embodiments, a phospholipid useful in the present invention is DSPC (1,2-dioctadecanoyl-sn-glycero-3-phosphocholine), or analog thereof, with a cyclic moiety in place of the glyceride moiety. In certain embodiments, the compound of Formula (IX) is of Formula (IX-b):

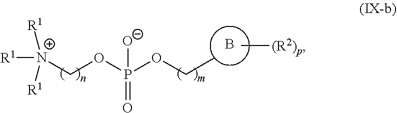

or a salt thereof.

In certain embodiments, the compound of Formula (IX-b) is of Formula (IX-b-1):

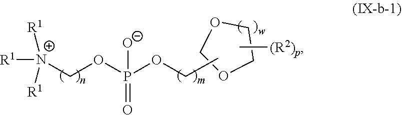

or a salt thereof, wherein:
w is 0, 1, 2, or 3.

In certain embodiments, the compound of Formula (IX-b) is of Formula (IX-b-2):

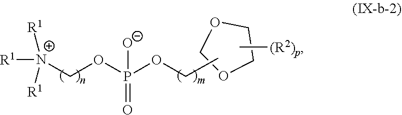

or a salt thereof.

In certain embodiments, the compound of Formula (IX-b) is of Formula (IX-b-3):

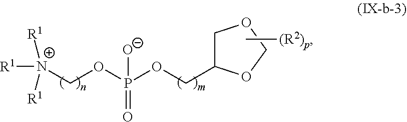

or a salt thereof.

In certain embodiments, the compound of Formula (IX-b) is of Formula (IX-b-4):

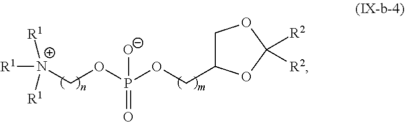

or a salt thereof.

In certain embodiments, the compound of Formula (IX-b) is one of the following:

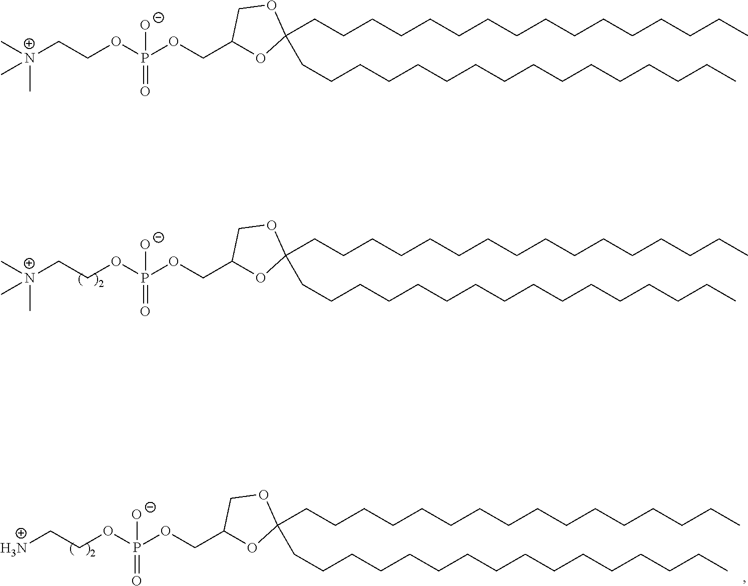

or salts thereof.

### (ii) Phospholipid Tail Modifications

In certain embodiments, a phospholipid useful or potentially useful in the present invention comprises a modified tail. In certain embodiments, a phospholipid useful or potentially useful in the present invention is DSPC (1,2-dioctadecanoyl-sn-glycero-3-phosphocholine), or analog thereof, with a modified tail. As described herein, a “modified tail” may be a tail with shorter or longer aliphatic chains, aliphatic chains with branching introduced, aliphatic chains with substituents introduced, aliphatic chains wherein one or more methylenes are replaced by cyclic or heteroatom groups, or any combination thereof. For example, in certain embodiments, the compound of (IX) is of Formula (IX-a), or a salt thereof, wherein at least one instance of R₂ is each instance of R₂ is optionally substituted C₁₋₃₀ alkyl, wherein one or more methylene units of R₂ are independently replaced with optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═N^{R}N)—, —C(═N^{R}N) N(R^{N})—, —NR^{N}C(═N^{R}N)—, —NR^{N}C(═N^{R}N) N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N}) S(O)—, —S(O)N(R^{N})—, —N(R^{N}) S(O)N(R^{N})—, —OS(O)N(R^{N})—, —N(R^{N}) S(O)O—, —S(O)₂—, —N(R^{N}) S(O)₂—, —S(O)₂N(R^{N})—, —N(R^{N}) S(O)₂N(R^{N})—, —OS(O)₂N(R^{N})—, or —N(R^{N}) S(O)₂O—.

In certain embodiments, the compound of Formula (IX) is of Formula (IX-c):

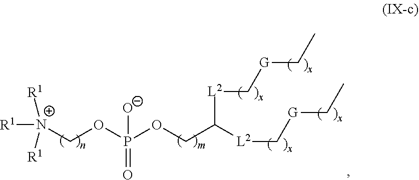

or a salt thereof, wherein:
each x is independently an integer between 0-30, inclusive; and
each instance is G is independently selected from the group consisting of optionally substituted carbocyclylene, optionally substituted heterocyclylene, optionally substituted arylene, optionally substituted heteroarylene, —N(R^{N})—, —O—, —S—, —C(O)—, —C(O)N(R^{N})—, —NR^{N}C(O)—, —NR^{N}C(O)N(R^{N})—, —C(O)O—, —OC(O)—, —OC(O)O—, —OC(O)N(R^{N})—, —NR^{N}C(O)O—, —C(O)S—, —SC(O)—, —C(═N^{R}N)—, —C(═N^{R}N) N(R^{N})—, —NR^{N}C(═N^{R}N)—, —NR^{N}C(═N^{R}N) N(R^{N})—, —C(S)—, —C(S)N(R^{N})—, —NR^{N}C(S)—, —NR^{N}C(S)N(R^{N})—, —S(O)—, —OS(O)—, —S(O)O—, —OS(O)O—, —OS(O)₂—, —S(O)₂O—, —OS(O)₂O—, —N(R^{N}) S(O)—, —S(O)N(R^{N})—, —N(R^{N}) S(O)N(R^{N})—, —OS(O)N(R^{N})—, —N(R^{N}) S(O)O—, —S(O)₂—, —N(R^{N}) S(O)₂—, —S(O)₂N(R^{N})—, —N(R^{N}) S(O)₂N(R^{N})—, —OS(O)₂N(R^{N})—, or —N(R^{N}) S(O)₂O—. Each possibility represents a separate embodiment of the present invention.

In certain embodiments, the compound of Formula (IX-c) is of Formula (IX-c-1):

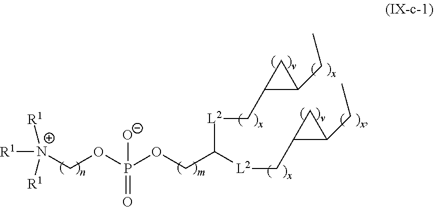

or salt thereof, wherein:
each instance of v is independently 1, 2, or 3.

In certain embodiments, the compound of Formula (IX-c) is of Formula (IX-c-2):

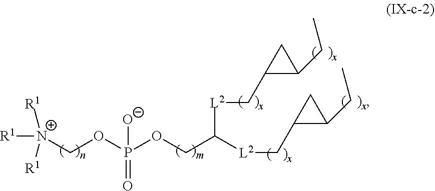

or a salt thereof.

In certain embodiments, the compound of Formula (IX-c) is of the following formula:

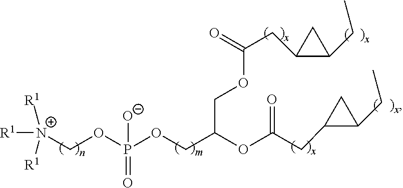

or a salt thereof.

In certain embodiments, the compound of Formula (IX-c) is the following:

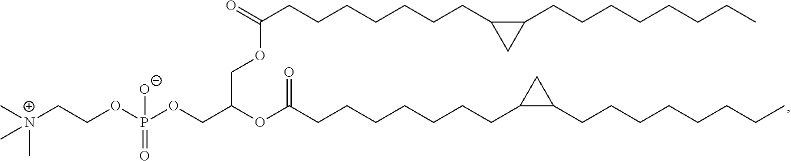 [SMILES: CCCCCCCCC1CC1CCCCCCCC(=O)OCC(COP(=O)([O-])OCC[N+](C)(C)C)OC(=O)CCCCCCCC1CC1CCCCCCCC]

or a salt thereof.

In certain embodiments, the compound of Formula (IX-c) is of Formula (IX-c-3):

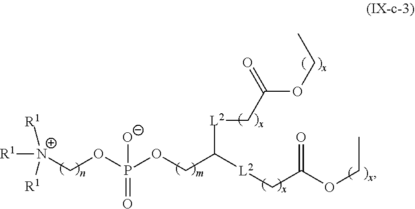

or a salt thereof.

In certain embodiments, the compound of Formula (IX-c) is of the following formulae:

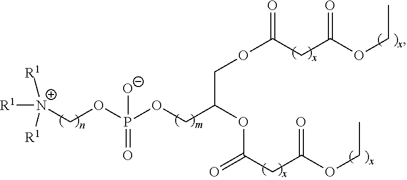

or a salt thereof.

In certain embodiments, the compound of Formula (IX-c) is the following:

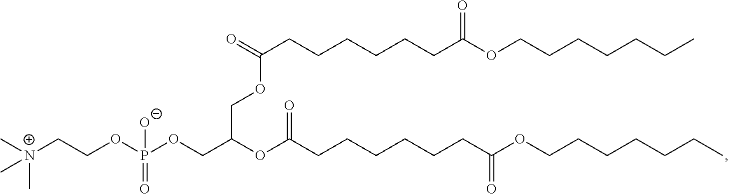 [SMILES: CCCCCCCOC(=O)CCCCCCC(=O)OCC(COP(=O)([O-])OCC[N+](C)(C)C)OC(=O)CCCCCCC(=O)OCCCCCCC]

or a salt thereof.

In certain embodiments, a phospholipid useful or potentially useful in the present invention comprises a modified phosphocholine moiety, wherein the alkyl chain linking the quaternary amine to the phosphoryl group is not ethylene (e.g., n is not 2). Therefore, in certain embodiments, a phospholipid useful or potentially useful in the present invention is a compound of Formula (IX), wherein n is 1, 3, 4, 5, 6, 7, 8, 9, or 10. For example, in certain embodiments, a compound of Formula (IX) is of one of the following formulae:

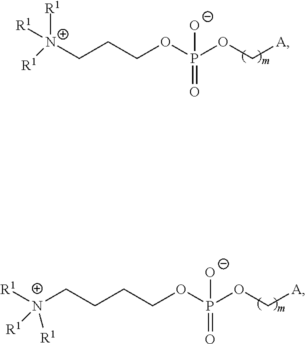

or a salt thereof.

In certain embodiments, a compound of Formula (IX) is one of the following:

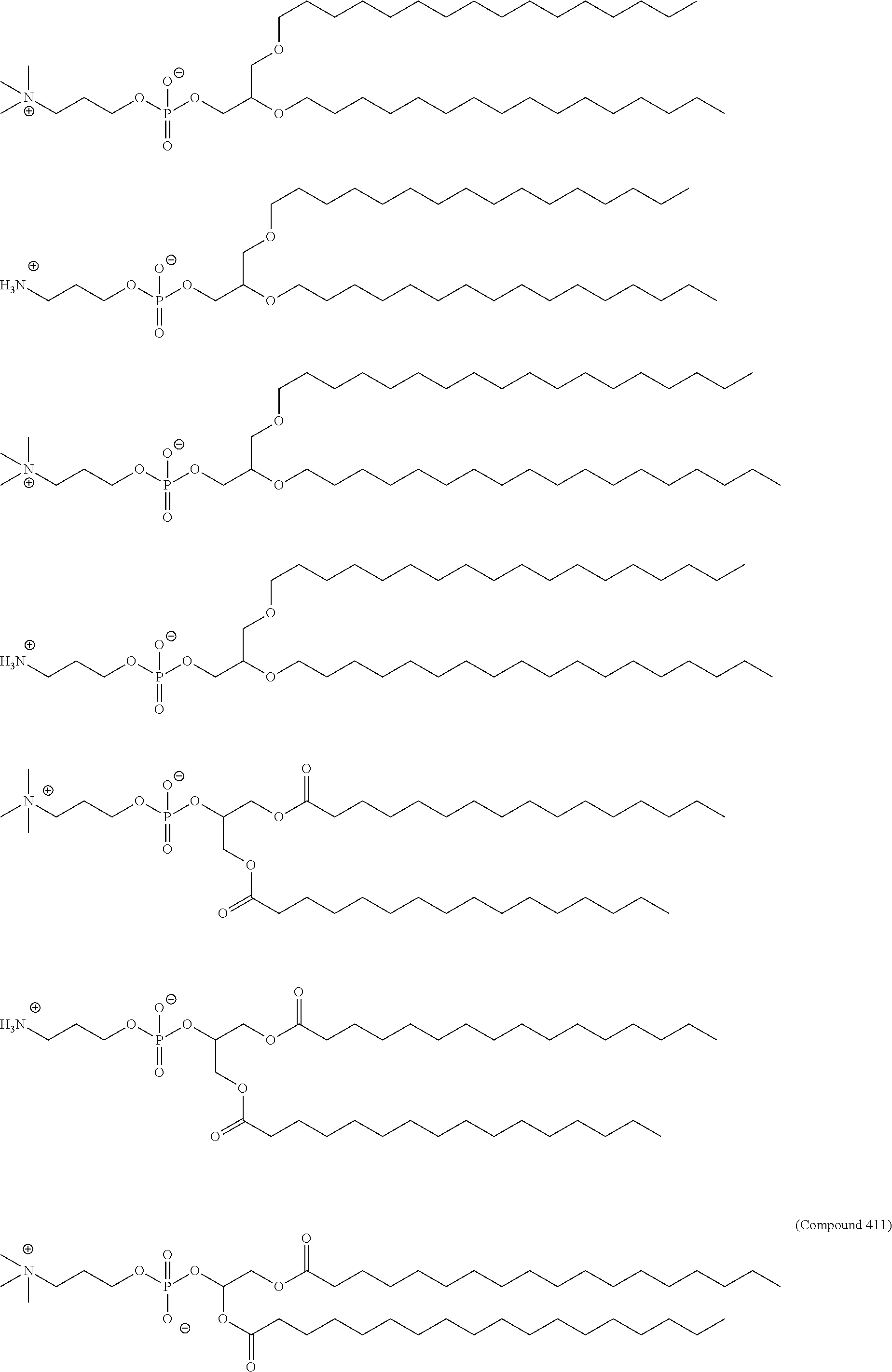

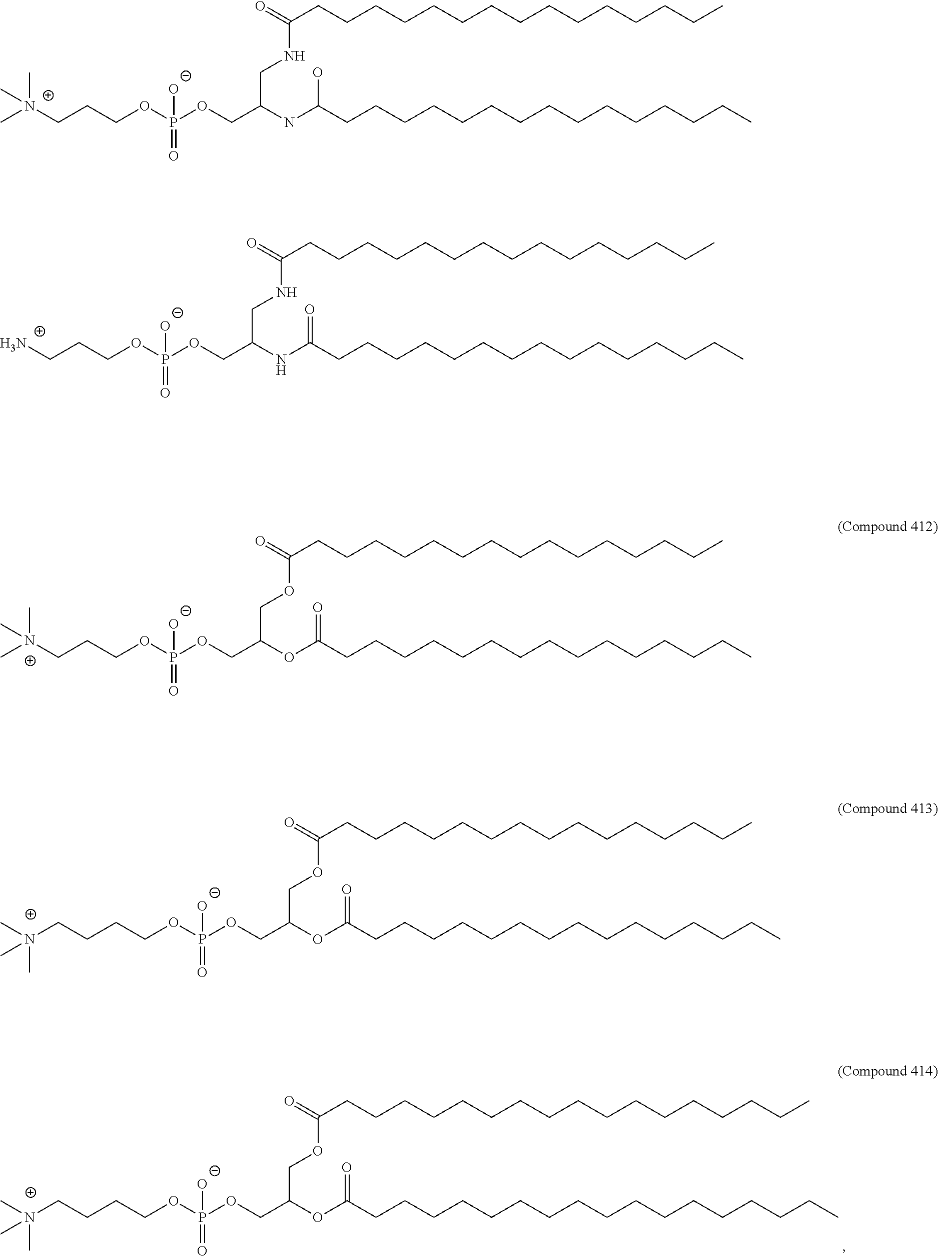

or salts thereof.
   c. Alternative lipids

In certain embodiments, an alternative lipid is used in place of a phospholipid of the invention. Non-limiting examples of such alternative lipids include the following:

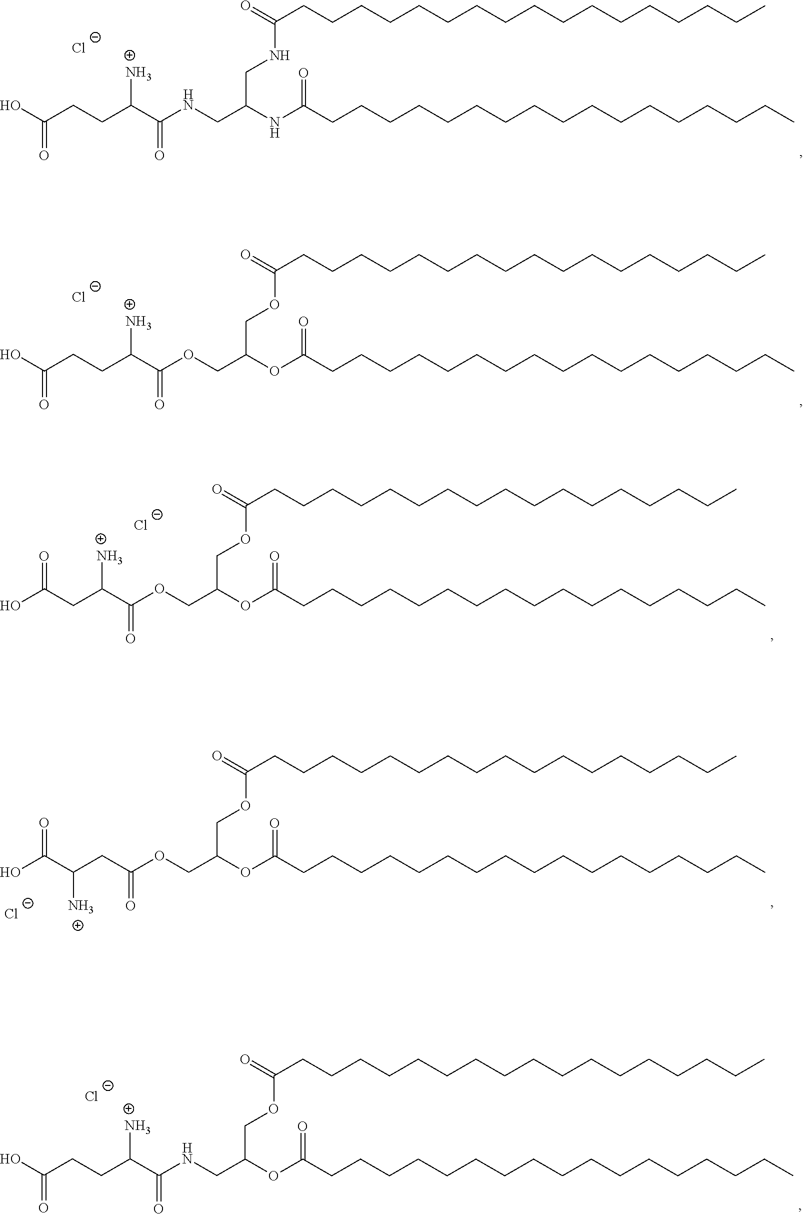

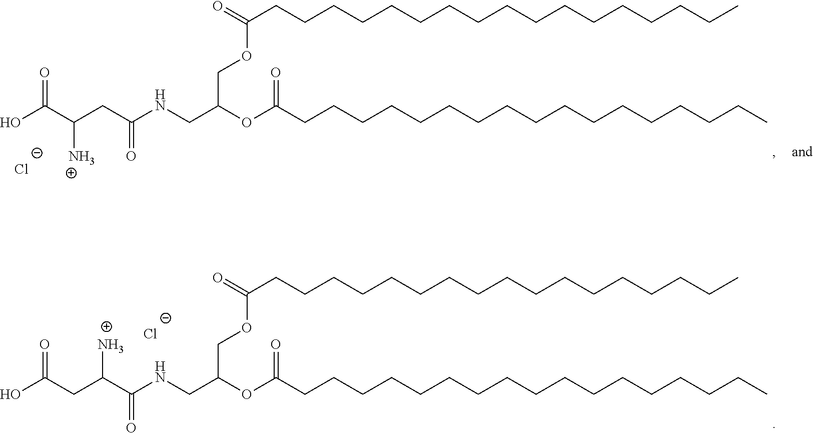 [SMILES: CCCCCCCCCCCCCCCCCC(=O)OCC(CNC(=O)CC([NH3+])C(=O)O)OC(=O)CCCCCCCCCCCCCCCCC ; [Cl-] ; CCCCCCCCCCCCCCCCCC(=O)OCC(CNC(=O)C([NH3+])CC(=O)O)OC(=O)CCCCCCCCCCCCCCCCC ; [Cl-]]

d. Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol. Examples of structural lipids include, but are not limited to, the following:

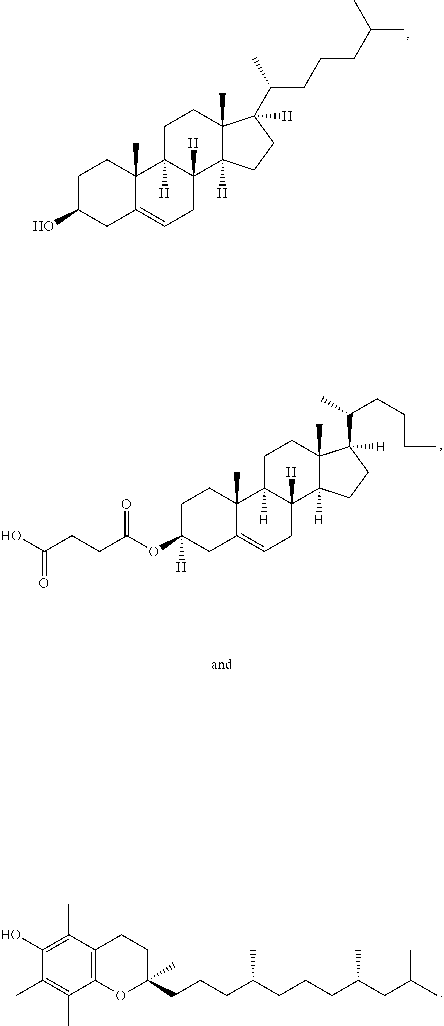

In one embodiment, the amount of the structural lipid (e.g., an sterol such as cholesterol) in the lipid composition of a pharmaceutical composition disclosed herein ranges from about 20 mol % to about 60 mol %, from about 25 mol % to about 55 mol %, from about 30 mol % to about 50 mol %, or from about 35 mol % to about 45 mol %.

In one embodiment, the amount of the structural lipid (e.g., an sterol such as cholesterol) in the lipid composition disclosed herein ranges from about 25 mol % to about 30 mol %, from about 30 mol % to about 35 mol %, or from about 35 mol % to about 40 mol %.

In one embodiment, the amount of the structural lipid (e.g., a sterol such as cholesterol) in the lipid composition disclosed herein is about 24 mol %, about 29 mol %, about 34 mol %, or about 39 mol %.

In some embodiments, the amount of the structural lipid (e.g., an sterol such as cholesterol) in the lipid composition disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 mol %.

e. Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids. For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino (polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A₂, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments the PEG-modified lipids are a modified form of PEG DMG. PEG-DMG has the following structure:

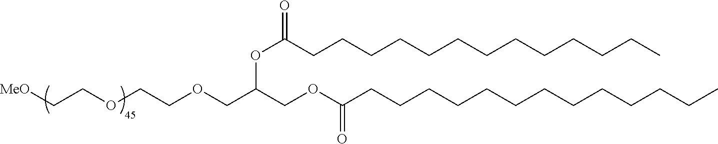

In one embodiment, PEG lipids useful in the present invention can be PEGylated lipids described in International Publication No. WO2012099755, the contents of which is herein incorporated by reference in its entirety. Any of these exemplary PEG lipids described herein may be modified to comprise a hydroxyl group on the PEG chain. In certain embodiments, the PEG lipid is a PEG-OH lipid. As generally defined herein, a “PEG-OH lipid” (also referred to herein as “hydroxy-PEGylated lipid”) is a PEGylated lipid having one or more hydroxyl (—OH) groups on the lipid. In certain embodiments, the PEG-OH lipid includes one or more hydroxyl groups on the PEG chain. In certain embodiments, a PEG-OH or hydroxy-PEGylated lipid comprises an —OH group at the terminus of the PEG chain. Each possibility represents a separate embodiment of the present invention.

In certain embodiments, a PEG lipid useful in the present invention is a compound of Formula (VII). Provided herein are compounds of Formula (VII):

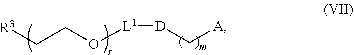

or salts thereof, wherein:
R₃ is —OR^{O};
R^{O} is hydrogen, optionally substituted alkyl, or an oxygen protecting group;
r is an integer between 1 and 100, inclusive;
L1 is optionally substituted C₁₋₁₀ alkylene, wherein at least one methylene of the optionally substituted C₁₋₁₀ alkylene is independently replaced with optionally substituted carbocyclylene, optionally substituted heterocyclylene, optionally substituted arylene, optionally substituted heteroarylene, O, N(R^{N}), S, C(O), C(O)N(R^{N}), NR^{N}C(O), C(O)O, —OC(0), OC(O)O, OC(O)N(R^{N}), NR^{N}C(O)O, or NR^{N}C(O)N(R^{N});
D is a moiety obtained by click chemistry or a moiety cleavable under physiological conditions;
m is 0, 1, 2, 3, 4, 5, 6, 7, 8, 9, or 10;
A is of the formula:

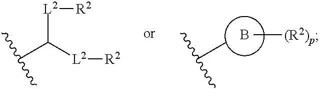

each instance of L2 is independently a bond or optionally substituted C₁₋₆ alkylene, wherein one methylene unit of the optionally substituted C₁₋₆ alkylene is optionally replaced with O, N(R^{N}), S, C(O), C(O)N(R^{N}), NR^{N}C(O), C(O)O, OC(O), OC(O)O, —OC(O)N(R^{N}), NR^{N}C(O)O, or NR^{N}C(O)N(R^{N});
each instance of R₂ is independently optionally substituted C₁₋₃₀ alkyl, optionally substituted C₁₋₃₀ alkenyl, or optionally substituted C₁₋₃₀ alkynyl; optionally wherein one or more methylene units of R₂ are independently replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), —NR^{N}C(O)N(R^{N}), C(O)O, OC(O), OC(O)O, OC(O)N(R^{N}), NR^{N}C(O)O, C(O) S, SC(O), —C(═N^{R}N), C(═N^{R}N) N(R^{N}), NR^{N}C(═N^{R}N), NR^{N}C(═N^{R}N) N(R^{N}), C(S), C(S)N(R^{N}), NR^{N}C(S), NR^{N}C(S)N(R^{N}), S(O), OS(O), S(O)O, OS(O)O, OS(O)₂, S(O)₂O, OS(O)₂O, N(R^{N}) S(O), —S(O)N(R^{N}), N(R^{N}) S(O)N(R^{N}), OS(O)N(R^{N}), N(R^{N}) S(O)O, S(O)₂, N(R^{N}) S(O)₂, S(O)₂N(R^{N}), N(R^{N}) S(O)₂N(R^{N}), OS(O)₂N(R^{N}), or N(R^{N}) S(O)₂O;
each instance of R^{N} is independently hydrogen, optionally substituted alkyl, or a nitrogen protecting group;
Ring B is optionally substituted carbocyclyl, optionally substituted heterocyclyl, optionally substituted aryl, or optionally substituted heteroaryl; and
p is 1 or 2.

In certain embodiments, the compound of Formula (VII) is a PEG-OH lipid (i.e., R₃ is —OR^{O}, and R^{O} is hydrogen). In certain embodiments, the compound of Formula (VII) is of Formula (VII—OH):

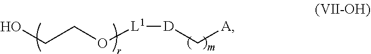

or a salt thereof.

In certain embodiments, D is a moiety obtained by click chemistry (e.g., triazole). In certain embodiments, the compound of Formula (VII) is of Formula (VII-a-1) or

(VII-a-2):

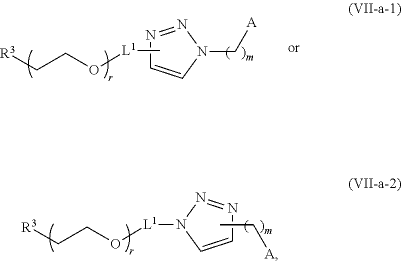

or a salt thereof.

In certain embodiments, the compound of Formula (VII) is of one of the following formulae:

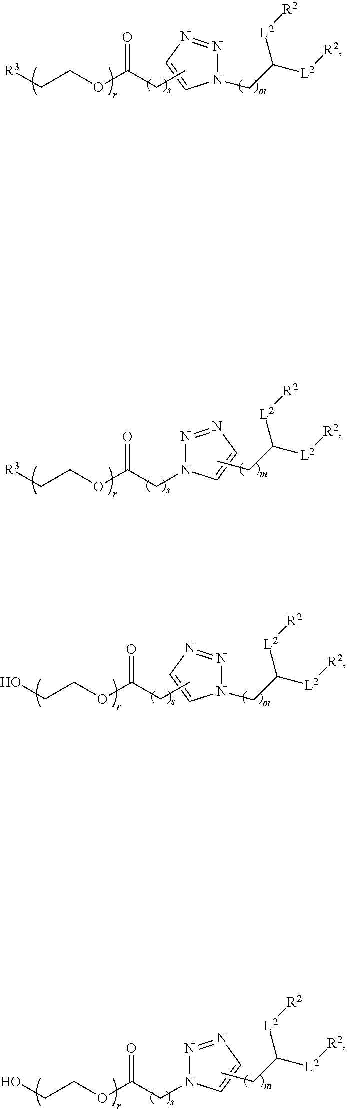

or a salt thereof, wherein
s is 0, 1, 2, 3, 4, 5, 6, 7, 8, 9, or 10.

In certain embodiments, the compound of Formula (VII) is of one of the following formulae:

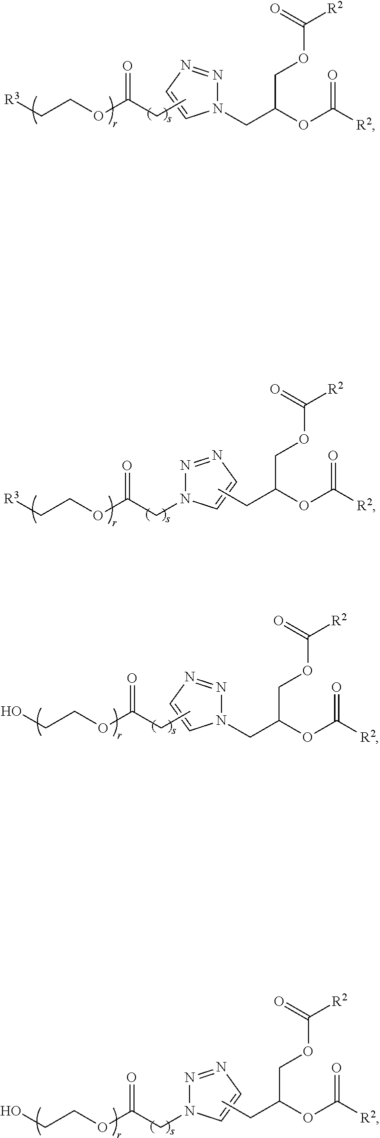

or a salt thereof.
In certain embodiments, a compound of Formula (VII) is of one of the following formulae:

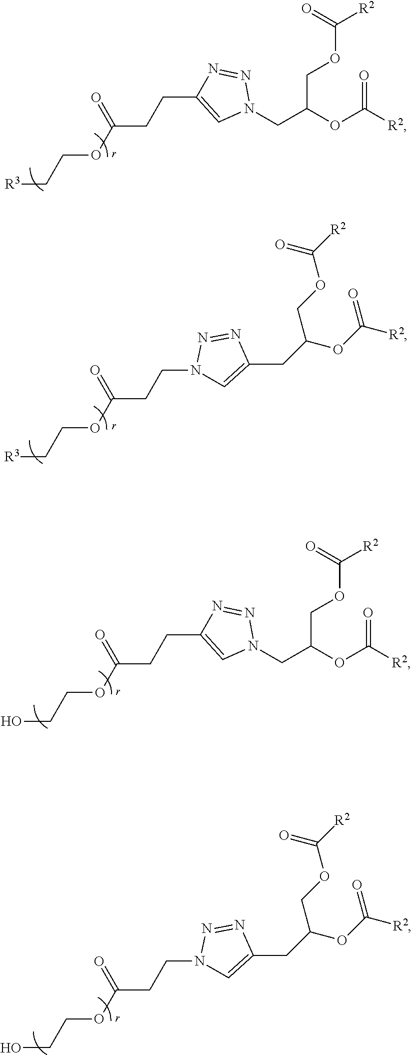

or a salt thereof.

In certain embodiments, a compound of Formula (VII) is of one of the following formulae:

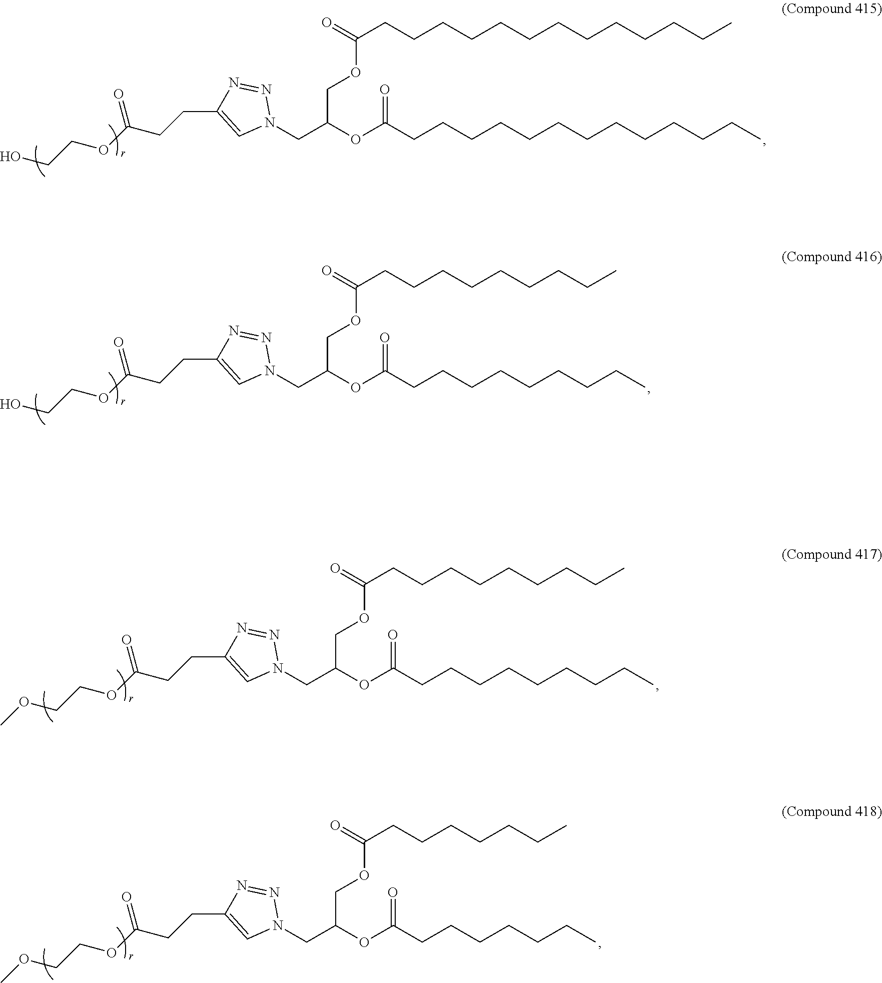

or a salt thereof.

In certain embodiments, D is a moiety cleavable under physiological conditions (e.g., ester, amide, carbonate, carbamate, urea). In certain embodiments, a compound of Formula (VII) is of Formula (VII-b-1) or (VII-b-2):

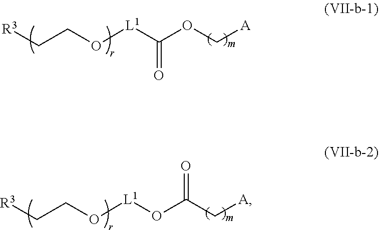

or a salt thereof.

In certain embodiments, a compound of Formula (VII) is of Formula (VII-b-1-OH) or (VII-b-2-OH):

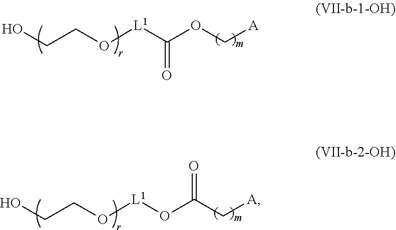

or a salt thereof.

In certain embodiments, the compound of Formula (VII) is of one of the following formulae:

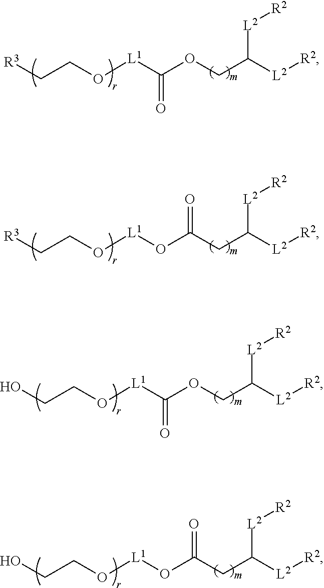

or a salt thereof.

In certain embodiments, a compound of Formula (VII) is of one of the following formulae:

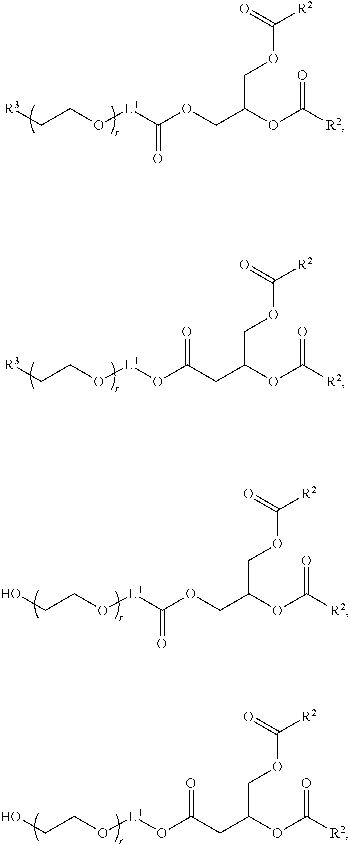

or a salt thereof.

In certain embodiments, a compound of Formula (VII) is of one of the following formulae:

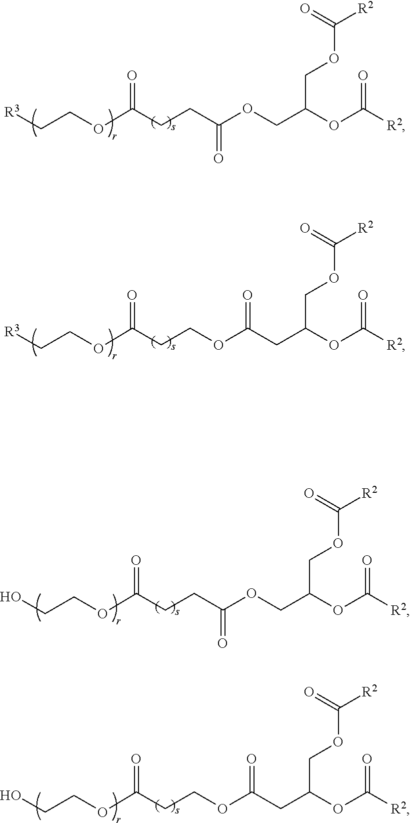

or a salt thereof.

In certain embodiments, a compound of Formula (VII) is of one of the following formulae:

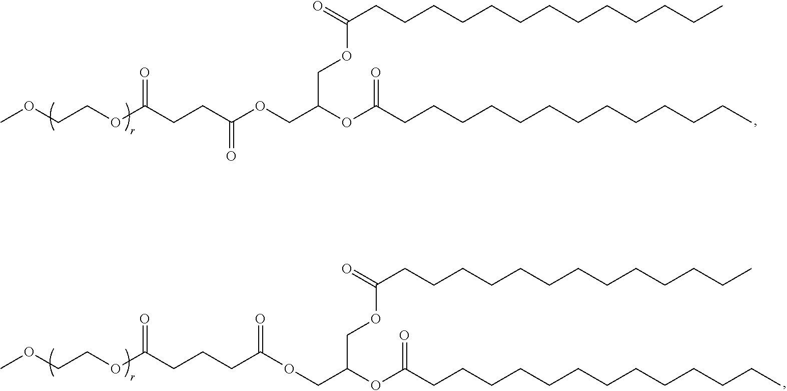

or salts thereof.

In certain embodiments, a PEG lipid useful in the present invention is a PEGylated fatty acid. In certain embodiments, a PEG lipid useful in the present invention is a compound of Formula (VIII). Provided herein are compounds of Formula (VIII):

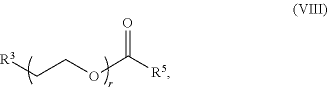

or a salts thereof, wherein:
R³ is —OR^{O};
R^{O} is hydrogen, optionally substituted alkyl or an oxygen protecting group; r is an integer between 1 and 100, inclusive;
R⁵ is optionally substituted C₁₀₋₄₀ alkyl, optionally substituted C₁₀₋₄₀ alkenyl, or optionally substituted C₁₀₋₄₀ alkynyl; and optionally one or more methylene groups of R⁵ are replaced with optionally substituted carbocyclylene, optionally substituted heterocyclylene, optionally substituted arylene, optionally substituted heteroarylene, N(R^{N}), O, S, C(O), C(O)N(R^{N}), NR^{N}C(O), NR^{N}C(O)N(R^{N}), C(O)O, OC(O), OC(O)O, OC(O)N(R^{N}), NR^{N}C(O)O, C(O) S, SC(O), C(═N^{R}N), C(═N^{R}N) N(R^{N}), NR^{N}C(═N^{R}N), NR^{N}C(═N^{R}N) N(R^{N}),
C(S), C(S)N(R^{N}), NR^{N}C(S), NR^{N}C(S)N(R^{N}), S(O), OS(O), S(O)O, OS(O)O, OS(O)₂, —S(O)₂O, OS(O)₂O, N(R^{N}) S(O), S(O)N(R^{N}), N(R^{N}) S(O)N(R^{N}), OS(O)N(R^{N}), N(R^{N}) S(O)O, S(O)₂, N(R^{N}) S(O)₂, S(O)₂N(R^{N}), N(R^{N}) S(O)₂N(R^{N}), OS(O)₂N(R^{N}), or N(R^{N}) S(O)₂O; and
each instance of R^{N} is independently hydrogen, optionally substituted alkyl, or a nitrogen protecting group.

In certain embodiments, the compound of Formula (VIII) is of Formula (VIII—OH):

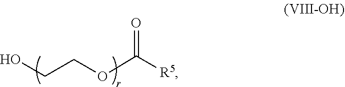

or a salt thereof. In some embodiments, r is 45.

In certain embodiments, a compound of Formula (VIII) is of one of the following formulae:

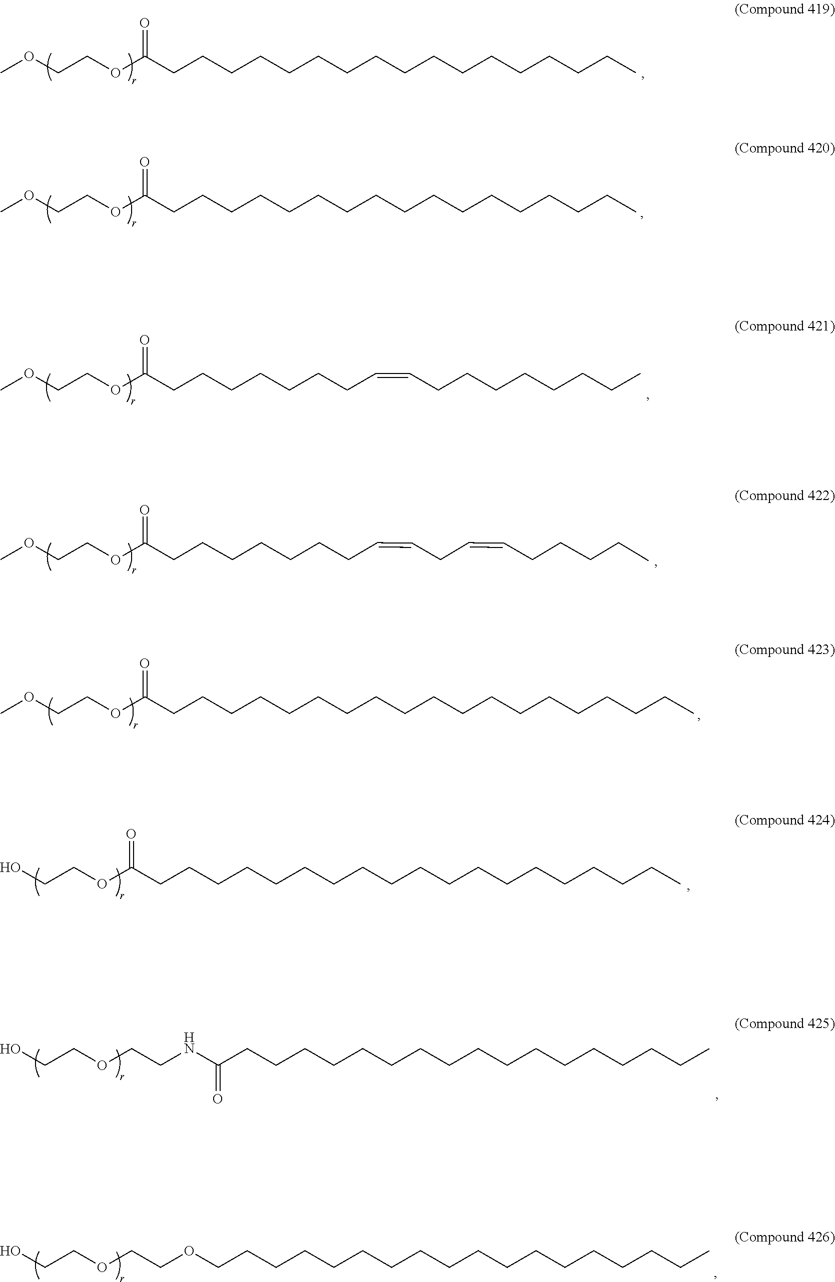

or a salt thereof. In some embodiments, r is 45.

In yet other embodiments the compound of Formula (VIII) is:

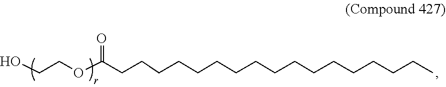

or a salt thereof.

In one embodiment, the compound of Formula (VIII) is

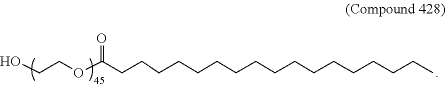

In one embodiment, the amount of PEG-lipid in the lipid composition of a pharmaceutical composition disclosed herein ranges from about 0.1 mol % to about 5 mol %, from about 0.5 mol % to about 5 mol %, from about 1 mol % to about 5 mol %, from about 1.5 mol % to about 5 mol %, from about 2 mol % to about 5 mol % mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition disclosed herein is about 2 mol %. In one embodiment, the amount of PEG-lipid in the lipid composition disclosed herein is about 1.5 mol %.

In one embodiment, the amount of PEG-lipid in the lipid composition disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

In some aspects, the lipid composition of the pharmaceutical compositions disclosed herein does not comprise a PEG-lipid.

f. Other Ionizable Amino Lipids

The lipid composition of the pharmaceutical composition disclosed herein can comprise one or more ionizable amino lipids in addition to or instead of a lipid according to Formula (I), (II), (III), (IV), (V), or (VI).

Ionizable lipids can be selected from the non-limiting group consisting of 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25-ditridecyl-15,18,21,24-tetraaza-octatriacontane (KL25), 1,2-dilinoleyloxy-N,N-dimethylaminopropane (DLin-DMA), 2,2-dilinoleyl-4-dimethylaminomethyl-[1,3]-dioxolane (DLin-K-DMA), heptatriaconta-6,9,28,31-tetraen-19-yl 4-(dimethylamino) butanoate (DLin-MC3-DMA), 2,2-dilinoleyl-4-(2-dimethylaminoethyl)-[1,3]-dioxolane (DLin-KC2-DMA), 1,2-dioleyloxy-N,N-dimethylaminopropane (DODMA), (13Z,165Z)—N,N-dimethyl-3-nonydocosa-13-16-dien-1-amine (L608), 2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA), (2R)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2R)), and (2S)-2-({8-[(3β)-cholest-5-en-3-yloxy]octyl}oxy)-N,N-dimethyl-3-[(9Z,12Z)-octadeca-9,12-dien-1-yloxy]propan-1-amine (Octyl-CLinDMA (2S)). In addition to these, an ionizable amino lipid can also be a lipid including a cyclic amine group.

Ionizable lipids can also be the compounds disclosed in International Publication No. WO 2017/075531 A1, hereby incorporated by reference in its entirety. For example, the ionizable amino lipids include, but not limited to:

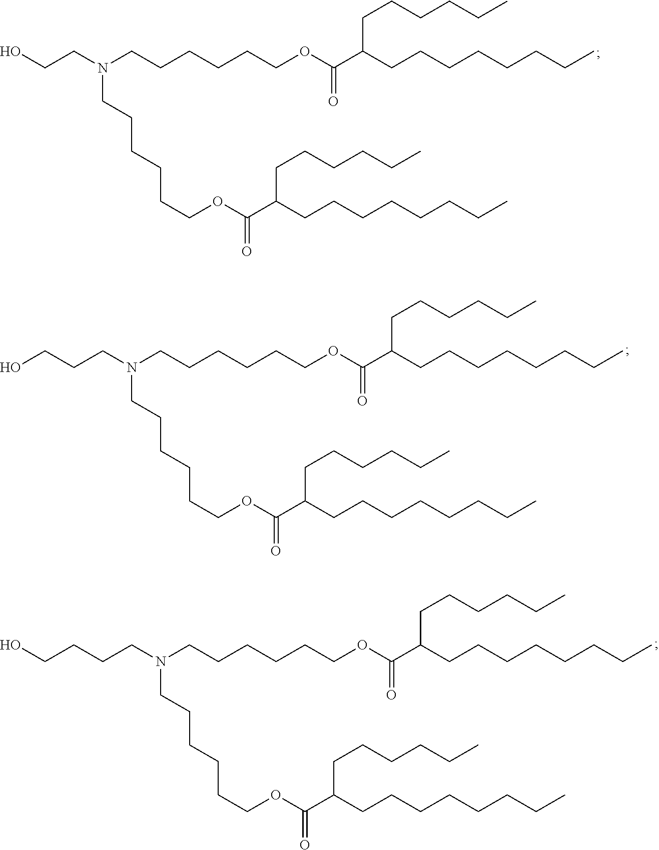 [SMILES: CCCCCCCCC(CCCCCC)C(=O)OCCCCCCN(CCO)CCCCCCOC(=O)C(CCCCCC)CCCCCCCC ; CCCCCCCCC(CCCCCC)C(=O)OCCCCCCN(CCCO)CCCCCCOC(=O)C(CCCCCC)CCCCCCCC ; CCCCCCCCC(CCCCCC)C(=O)OCCCCCCN(CCCCO)CCCCCCOC(=O)C(CCCCCC)CCCCCCCC]

and any combination thereof.

Ionizable lipids can also be the compounds disclosed in International Publication No. WO 2015/199952 A1, hereby incorporated by reference in its entirety. For example, the ionizable amino lipids include, but not limited to:

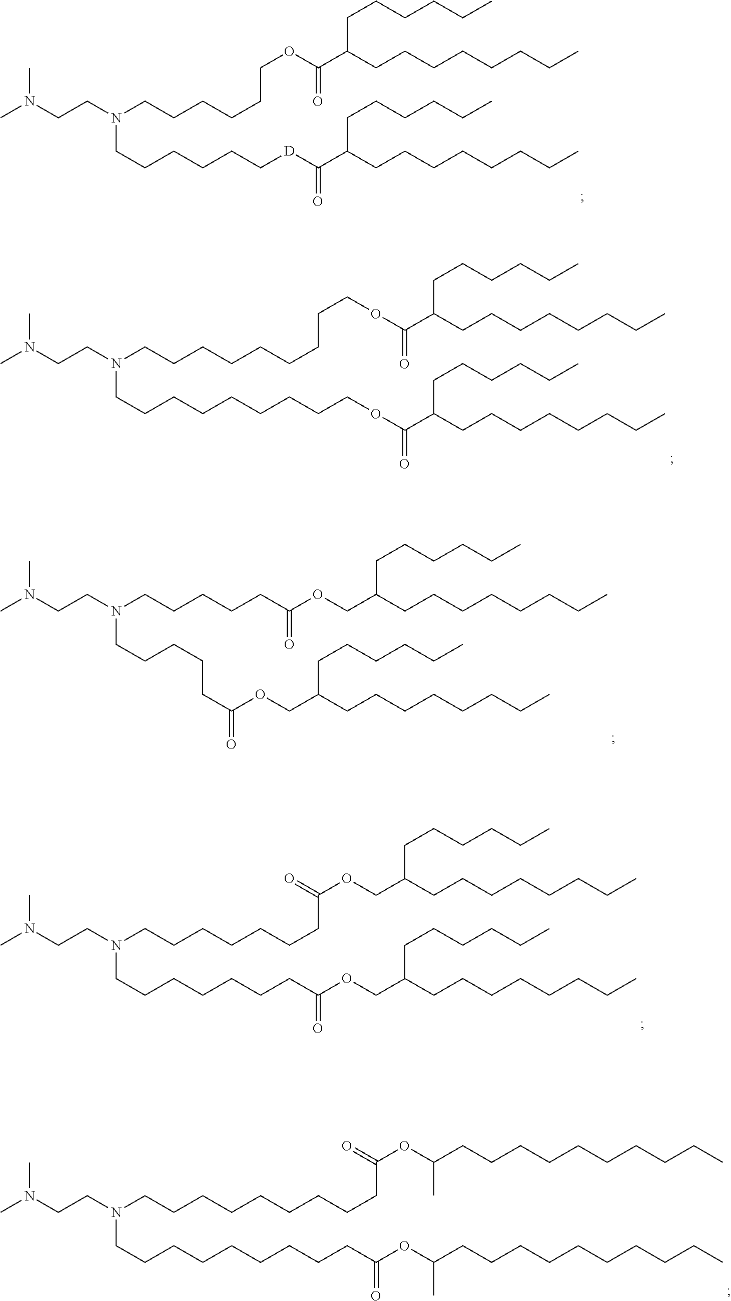

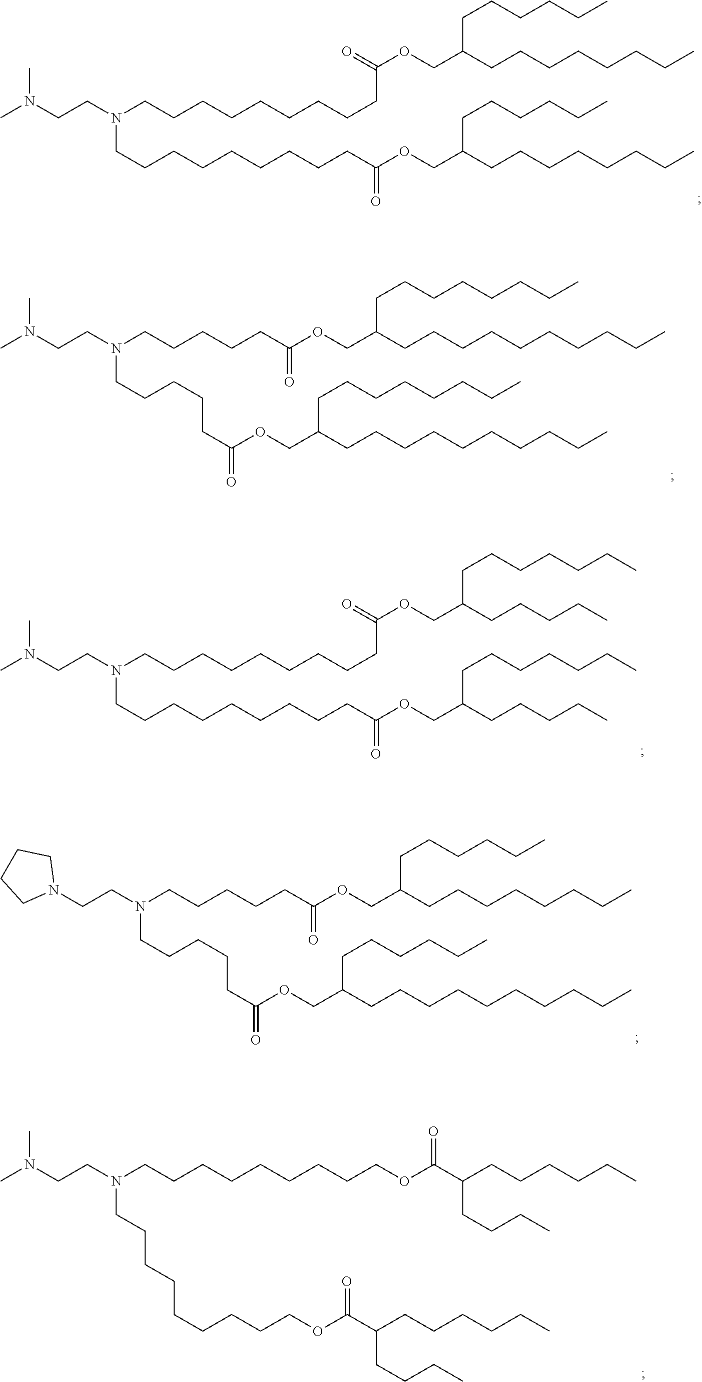

and any combination thereof.
   g. Nanoparticle Compositions

The lipid composition of a pharmaceutical composition disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described by U.S. Patent Application Publication No. 2005/0222064. Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates. The ratio between the lipid composition and the polynucleotide range can be from about 10:1 to about 60:1 (wt/wt).

In some embodiments, the ratio between the lipid composition and the polynucleotide can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the polynucleotide encoding a therapeutic agent is about 20:1 or about 15:1.

In one embodiment, the lipid nanoparticles described herein can comprise polynucleotides (e.g., mRNA) in a lipid: polynucleotide weight ratio of 5:1, 10:1, 15:1, 20:1, 25:1, 30:1, 35:1, 40:1, 45:1, 50:1, 55:1, 60:1 or 70:1, or a range or any of these ratios such as, but not limited to, 5:1 to about 10:1, from about 5:1 to about 15:1, from about 5:1 to about 20:1, from about 5:1 to about 25:1, from about 5:1 to about 30:1, from about 5:1 to about 35:1, from about 5:1 to about 40:1, from about 5:1 to about 45:1, from about 5:1 to about 50:1, from about 5:1 to about 55:1, from about 5:1 to about 60:1, from about 5:1 to about 70:1, from about 10:1 to about 15:1, from about 10:1 to about 20:1, from about 10:1 to about 25:1, from about 10:1 to about 30:1, from about 10:1 to about 35:1, from about 10:1 to about 40:1, from about 10:1 to about 45:1, from about 10:1 to about 50:1, from about 10:1 to about 55:1, from about 10:1 to about 60:1, from about 10:1 to about 70:1, from about 15:1 to about 20:1, from about 15:1 to about 25:1, from about 15:1 to about 30:1, from about 15:1 to about 35:1, from about 15:1 to about 40:1, from about 15:1 to about 45:1, from about 15:1 to about 50:1, from about 15:1 to about 55:1, from about 15:1 to about 60:1 or from about 15:1 to about 70:1.

In one embodiment, the lipid nanoparticles described herein can comprise the polynucleotide in a concentration from approximately 0.1 mg/ml to 2 mg/ml such as, but not limited to, 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2.0 mg/ml or greater than 2.0 mg/ml.

In some embodiments, the pharmaceutical compositions disclosed herein are formulated as lipid nanoparticles (LNP). Accordingly, the present disclosure also provides nanoparticle compositions comprising (i) a lipid composition comprising a delivery agent such as a compound of Formula (I) or (III) as described herein, and (ii) a polynucleotide encoding a polypeptide of interest. In such nanoparticle composition, the lipid composition disclosed herein can encapsulate the polynucleotide encoding a polypeptide of interest.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In some embodiments, the nanoparticle compositions of the present disclosure comprise at least one compound according to Formula (I), (III), (IV), (V), or (VI). For example, the nanoparticle composition can include one or more of Compounds 1-147, or one or more of Compounds 1-342. Nanoparticle compositions can also include a variety of other components. For example, the nanoparticle composition may include one or more other lipids in addition to a lipid according to Formula (I), (II), (III), (IV), (V), or (VI), such as (i) at least one phospholipid, (ii) at least one structural lipid, (iii) at least one PEG-lipid, or (iv) any combination thereof. Inclusion of structural lipid can be optional, for example when lipids according to formula III are used in the lipid nanoparticle compositions of the invention.

In some embodiments, the nanoparticle composition comprises a compound of Formula (I), (e.g., Compounds 18, 25, 26 or 48). In some embodiments, the nanoparticle composition comprises a compound of Formula (I) (e.g., Compounds 18, 25, 26 or 48) and a phospholipid (e.g., DSPC).

In some embodiments, the nanoparticle composition comprises a compound of Formula (III) (e.g., Compound 236). In some embodiments, the nanoparticle composition comprises a compound of Formula (III) (e.g., Compound 236) and a phospholipid (e.g., DOPE or DSPC).

In some embodiments, the nanoparticle composition comprises a lipid composition consisting or consisting essentially of compound of Formula (I) (e.g., Compounds 18, 25, 26 or 48). In some embodiments, the nanoparticle composition comprises a lipid composition consisting or consisting essentially of a compound of Formula (I) (e.g., Compounds 18, 25, 26 or 48) and a phospholipid (e.g., DSPC).

In some embodiments, the nanoparticle composition comprises a lipid composition consisting or consisting essentially of compound of Formula (III) (e.g., Compound 236). In some embodiments, the nanoparticle composition comprises a lipid composition consisting or consisting essentially of a compound of Formula (III) (e.g., Compound 236) and a phospholipid (e.g., DOPE or DSPC).

In one embodiment, a lipid nanoparticle comprises an ionizable lipid, a structural lipid, a phospholipid, a PEG-modified lipid, and mRNA. In some embodiments, the LNP comprises an ionizable lipid, a PEG-modified lipid, a sterol and a phospholipid. In some embodiments, the LNP has a molar ratio of about 20-60% ionizable lipid: about 5-25% phospholipid: about 25-55% sterol; and about 0.5-15% PEG-modified lipid. In some embodiments, the LNP comprises a molar ratio of about 50% ionizable lipid, about 1.5% PEG-modified lipid, about 38.5% cholesterol and about 10% phospholipid. In some embodiments, the LNP comprises a molar ratio of about 55% ionizable lipid, about 2.5% PEG lipid, about 32.5% cholesterol and about 10% phospholipid. In some embodiments, the ionizable lipid is an ionizable amino lipid, the neutral lipid is a phospholipid, and the sterol is a cholesterol. In some embodiments, the LNP has a molar ratio of 50:38.5:10:1.5 of ionizable lipid: cholesterol: DSPC: PEG lipid. In some embodiments, the ionizable lipid is Compound 18 or Compound 236, and the PEG lipid is Compound 428 or PEG-DMG.

In some embodiments, the LNP has a molar ratio of 50:38.5:10:1.5 of Compound 18: Cholesterol: Phospholipid: Compound 428. In some embodiments, the LNP has a molar ratio of 50:38.5:10:1.5 of Compound 18: Cholesterol: DSPC: Compound 428. In some embodiments, the LNP has a molar ratio of 50:38.5:10:1.5 of Compound 18: Cholesterol: Phospholipid: PEG-DMG. In some embodiments, the LNP has a molar ratio of 50:38.5:10:1.5 of Compound 18: Cholesterol: DSPC: PEG-DMG.

In some embodiments, the LNP has a molar ratio of 50:38.5:10:1.5 of Compound 236: Cholesterol: Phospholipid: Compound 428. In some embodiments, the LNP has a molar ratio of 50:38.5:10:1.5 of Compound 236: Cholesterol: DSPC: Compound 428.

In some embodiments, the LNP has a molar ratio of 40:38.5:20:1.5 of Compound 18: Cholesterol: Phospholipid: Compound 428. In some embodiments, the LNP has a molar ratio of 40:38.5:20:1.5 of Compound 18: Cholesterol: DSPC: Compound 428. In some embodiments, the LNP has a molar ratio of 40:38.5:20:1.5 of Compound 18: Cholesterol: Phospholipid: PEG-DMG. In some embodiments, the LNP has a molar ratio of 40:38.5:20:1.5 of Compound 18: Cholesterol: DSPC: PEG-DMG.

In some embodiments, a nanoparticle composition can have the formulation of Compound 18: Phospholipid: Chol: Compound 428 with a mole ratio of 50:10:38.5:1.5. In some embodiments, a nanoparticle composition can have the formulation of Compound 18:DSPC:Chol: Compound 428 with a mole ratio of 50:10:38.5:1.5. In some embodiments, a nanoparticle composition can have the formulation of Compound 18: Phospholipid: Chol:PEG-DMG with a mole ratio of 50:10:38.5:1.5. In some embodiments, a nanoparticle composition can have the formulation of Compound 18:DSPC:Chol:PEG-DMG with a mole ratio of 50:10:38.5:1.5.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids leads them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipids. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

It should be understood that the terms “charged” or “charged moiety” does not refer to a “partial negative charge” or “partial positive charge” on a molecule. The terms “partial negative charge” and “partial positive charge” are given its ordinary meaning in the art. A “partial negative charge” may result when a functional group comprises a bond that becomes polarized such that electron density is pulled toward one atom of the bond, creating a partial negative charge on the atom. Those of ordinary skill in the art will, in general, recognize bonds that can become polarized in this way.

In some embodiments, the ionizable lipid is an ionizable amino lipid, sometimes referred to in the art as an “ionizable cationic lipid”. In one embodiment, the ionizable amino lipid may have a positively charged hydrophilic head and a hydrophobic tail that are connected via a linker structure.

In addition to these, an ionizable lipid may also be a lipid including a cyclic amine group.

In one embodiment, the ionizable lipid may be selected from, but not limited to, a ionizable lipid described in International Publication Nos. WO2013086354 and WO2013116126; the contents of each of which are herein incorporated by reference in their entirety.

In yet another embodiment, the ionizable lipid may be selected from, but not limited to, formula CLI-CLXXXXII of U.S. Pat. No. 7,404,969; each of which is herein incorporated by reference in their entirety.

In one embodiment, the lipid may be a cleavable lipid such as those described in International Publication No. WO2012170889, herein incorporated by reference in its entirety. In one embodiment, the lipid may be synthesized by methods known in the art and/or as described in International Publication Nos. WO2013086354; the contents of each of which are herein incorporated by reference in their entirety.

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

In some embodiments, the nanoparticle composition comprises a lipid composition consisting or consisting essentially of compound of Formula (I) (e.g., Compounds 18, 25, 26 or 48). In some embodiments, the nanoparticle composition comprises a lipid composition consisting or consisting essentially of a compound of Formula (I) (e.g., Compounds 18, 25, 26 or 48) and a phospholipid (e.g., DSPC or MSPC).

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of the polynucleotide.

As used herein, “size” or “mean size” in the context of nanoparticle compositions refers to the mean diameter of a nanoparticle composition.

In one embodiment, the polynucleotide encoding a polypeptide of interest are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a polynucleotide describes the amount of the polynucleotide that is encapsulated by or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of the polynucleotide in a solution containing the nanoparticle composition before and after breaking up the nanoparticle composition with one or more organic solvents or detergents.

Fluorescence can be used to measure the amount of free polynucleotide in a solution. For the nanoparticle compositions described herein, the encapsulation efficiency of a polynucleotide can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a polynucleotide present in a pharmaceutical composition disclosed herein can depend on multiple factors such as the size of the polynucleotide, desired target and/or application, or other properties of the nanoparticle composition as well as on the properties of the polynucleotide.

For example, the amount of an mRNA useful in a nanoparticle composition can depend on the size (expressed as length, or molecular mass), sequence, and other characteristics of the mRNA. The relative amounts of a polynucleotide in a nanoparticle composition can also vary.

The relative amounts of the lipid composition and the polynucleotide present in a lipid nanoparticle composition of the present disclosure can be optimized according to considerations of efficacy and tolerability. For compositions including an mRNA as a polynucleotide, the N:P ratio can serve as a useful metric.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition. In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1.

In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles comprising encapsulating a polynucleotide. Such method comprises using any of the pharmaceutical compositions disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16:940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

### Pharmaceutical Compositions

The present disclosure includes pharmaceutical compositions comprising an mRNA or a nanoparticle (e.g., a lipid nanoparticle) described herein, in combination with one or more pharmaceutically acceptable excipient, carrier or diluent. In particular embodiments, the mRNA is present in a nanoparticle, e.g., a lipid nanoparticle. In particular embodiments, the mRNA or nanoparticle is present in a pharmaceutical composition. In various embodiments, the one or more mRNA present in the pharmaceutical composition is encapsulated in a nanoparticle, e.g., a lipid nanoparticle. In particular embodiments, the molar ratio of the first mRNA to the second mRNA is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In particular embodiments, the molar ratio of the first mRNA to the second mRNA is greater than 1:1.

In some embodiments, a composition described herein comprises an mRNA encoding an antigen of interest (Ag) and an mRNA encoding a polypeptide that enhances an immune response to the antigen of interest (e.g., immune potentiator (IP), e.g., STING polypeptide) wherein the mRNA encoding the antigen of interest (Ag) and the mRNA encoding the polypeptide that enhances an immune response to the antigen of interest (e.g., immune potentiator, e.g., STING polypeptide) (IP) are formulated at an Ag:IP mass ratio of 1:1, 2:1, 3:1, 4:1, 5:1, 6:1, 7:1, 8:1, 9:1, 10:1 or 20:1. Alternatively, the IP:Ag mass ratio can be, for example, 1:1, 1:2, 1:3, 1:4, 1:5, 1:6, 1:7, 1:8, 1:9, 1:10 or 1:20. In some embodiments, the composition is formulated at an Ag:IP mass ratio of 1:1. 1.25:1, 1.50:1, 1.75:1, 2.0:1, 2.25:1, 2.50:1, 2.75:1, 3.0:1, 3.25:1, 3.50:1, 3.75:1, 4.0:1, 4.25:1, 4.50:1, 4.75:1 or 5:1 of mRNA encoding the antigen of interest to the mRNA encoding the polypeptide that enhances an immune to the antigen of interest (e.g., immune potentiator, e.g., STING polypeptide). In some embodiments, the composition is formulated at a mass ratio of 5:1 of mRNA encoding the antigen of interest to the mRNA encoding the polypeptide that enhances an immune to the antigen of interest (e.g., immune potentiator, e.g., STING polypeptide) (Ag:IP ratio or 5:1; or alternatively, an IP:Ag ratio of 1:5). In some embodiments, the composition is formulated at a mass ratio of 10:1 of mRNA encoding the antigen of interest to the mRNA encoding the polypeptide that enhances an immune to the antigen of interest (e.g., immune potentiator, e.g., STING polypeptide) (Ag:IP ratio of 10:1, or alternatively, an IP:Ag ratio of 1:10).

Coformulations that contain both an mRNA construct encoding an immune protentiator and an mRNA construct encoding an antigen of interest may be particularly beneficial for priming of CD8+ T cells and inducing antigen-specific immune responses (e.g., anti-tumor immunity). It has been reported in that art that direct activation of antigen-presenting cells (APCs) by pathogen-associated molecular patterns (PAMPs) is required for CD8+ T cell priming, whereas APCs indirectly activated by proinflammatory mediators were not effective in priming CD8+ T cells (Kratky, W. et al. (2011) Proc. Natl. Acad. Sci. USA 108:17414-17419). Accordingly, coformulation of mRNA constructs encoding an immune potentiator and an antigen of interest may be particularly beneficial for directly activating APCs and priming CD8+ T cells.

Pharmaceutical compositions may optionally include one or more additional active substances, for example, therapeutically and/or prophylactically active substances. Pharmaceutical compositions of the present disclosure may be sterile and/or pyrogen-free. General considerations in the formulation and/or manufacture of pharmaceutical agents may be found, for example, in Remington: The Science and Practice of Pharmacy 21^{st} ed., Lippincott Williams & Wilkins, 2005 (incorporated herein by reference in its entirety). In particular embodiments, a pharmaceutical composition comprises an mRNA and a lipid nanoparticle, or complexes thereof.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100%, e.g., between 0.5% and 70%, between 1% and 30%, between 5% and 80%, or at least 80% (w/w) active ingredient.

The mRNAs of the disclosure can be formulated using one or more excipients to: (1) increase stability; (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the disclosure can include one or more excipients, each in an amount that together increases the stability of the mRNA, increases cell transfection by the mRNA, increases the expression of a polypeptide encoded by the mRNA, and/or alters the release profile of a mRNA-encoded polypeptide. Further, the mRNAs of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, the formulations described herein may include at least one pharmaceutically acceptable salt. Examples of pharmaceutically acceptable salts that may be included in a formulation of the disclosure include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In some embodiments, the formulations described herein may contain at least one type of polynucleotide. As a non-limiting example, the formulations may contain 1, 2, 3, 4, 5 or more than 5 mRNAs described herein. In some embodiments, the formulations described herein may contain at least one mRNA encoding a polypeptide and at least one nucleic acid sequence such as, but not limited to, an siRNA, an shRNA, a snoRNA, and an miRNA.

Liquid dosage forms for e.g., parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and/or suspending agents. In certain embodiments for parenteral administration, compositions are mixed with solubilizing agents such as CREMAPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, pharmaceutical compositions including at least one mRNA described herein are administered to mammals (e.g., humans). Although the descriptions of pharmaceutical compositions provided herein are principally directed to pharmaceutical compositions which are suitable for administration to humans, it will be understood by the skilled artisan that such compositions are generally suitable for administration to any other animal, e.g., to a non-human mammal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys. In particular embodiments, a subject is provided with two or more mRNAs described herein. In particular embodiments, the first and second mRNAs are provided to the subject at the same time or at different times, e.g., sequentially. In particular embodiments, the first and second mRNAs are provided to the subject in the same pharmaceutical composition or formulation, e.g., to facilitate uptake of both mRNAs by the same cells.

The present disclosure also includes kits comprising a container comprising a mRNA encoding a polypeptide that enhances an immune response. In another embodiment, the kit comprises a container comprising a mRNA encoding a polypeptide that enhances an immune response, as well as one or more additional mRNAs encoding one or more antigens or interest. In other embodiments, the kit comprises a first container comprising the mRNA encoding a polypeptide that enhances an immune response and a second container comprising one or more mRNAs encoding one or more antigens of interest. In particular embodiments, the mRNAs for enhancing an immune response and the mRNA(s) encoding an antigen(s) are present in the same or different nanoparticles and/or pharmaceutical compositions. In particular embodiments, the mRNAs are lyophilized, dried, or freeze-dried.

### Methods of Enhancing Immune Responses

The disclosure provides a method for enhancing an immune response to an antigen of interest in a subject, e.g., a human subject. In one embodiment, the method comprises administering to the subject a composition of the disclosure (or lipid nanoparticle thereof, or pharmaceutical composition thereof) comprising at least one mRNA construct encoding:(i) at least one antigen of interest and (ii) a polypeptide that enhances an immune response against the antigen(s) of interest, such that an immune response to the antigen(s) of interest is enhanced. In one embodiment, enhancing an immune response comprises stimulating cytokine production. In another embodiment, enhancing an immune response comprises enhancing cellular immunity (T cell responses), such as stimulating antigen-specific CD8⁺ T cell activity, stimulating antigen-specific CD4⁺ T cell activity or increasing the percentage of “effector memory” CD62L^{lo} T cells. In another embodiment, enhancing an immune response comprises enhancing humoral immunity (B cell responses), such as stimulating antigen-specific antibody production.

In one embodiment of the method, the immune potentiator mRNA encodes a polypeptide that stimulates Type I interferon pathway signaling (e.g., the immune potentiator encodes a polypeptide such as STING, IRF3, IRF7 or any of the additional immune potentiators described herein). In various other embodiment of the method, the immune potentiator encodes a polypeptide that stimulates NFκB pathway signaling, stimulates an inflammatory response or stimulates dendritic cell development, activity or mobilization. In one embodiment, the method comprises administering to the subject an mRNA composition that stimulates dendritic cell development, activity or mobilization prior to administering to the subject an mRNA composition that stimulates Type I interferon pathway signaling. For example, the mRNA composition that stimulates dendritic cell development or activity can be administered 1-30 days, e.g., 3 days, 5 days, 7 days, 10 days, 14 days, 21 days, 28 days, prior to administering the mRNA composition that stimulates Type I interferon pathway signaling. Enhancement of an immune response in a subject against an antigen(s) of interest by an immune potentiator of the disclosure can be evaluated by a variety of methods established in the art for assessing immune responses, including but not limited to the methods described in the Examples. For example, in various embodiments, enhancement is evaluated by levels of intracellular staining (ICS) of CD8+ cells for IFN-γ or TNF-α, percentage of splenic or peripheral CD8b⁺ cells, or percentage of splenic or peripheral “effector memory” CD62L^{lo} cells.

It has been reported that the outcome of STING-mediated signaling can vary between different cell types, with T cells in particular exhibiting a stronger STING response as compared to other cell types (e.g., macrophages and dendritic cells), along with T cells exhibiting increased expression levels of STING (Gulen, M. F. et al. (2017) Nature Comm. 8 (1): 427). Thus, the magnitude of STING signaling can result in distinct effector responses, thereby allowing for adjustment and fine-tuning of STING-mediated responses depending on dosage, cell-type expression and/or co-formulation with an antigen of interest (e.g., Ag:STING ratio). Data described in the Examples indicates that there is a wide therapeutic window in which STING exhibits effectiveness in enhancing antigen-specific immune responses.

Compositions of the disclosure are administered to the subject at an effective amount. In general, an effective amount of the composition will allow for efficient production of the encoded polypeptide in the cell. Metrics for efficiency may include polypeptide translation (indicated by polypeptide expression), level of mRNA degradation, and immune response indicators.

### Methods of Inducing Immunogenic Cell Death

The invention provides methods of inducing immunogenic cell death in a cell, e.g., a mammalian cell. In one embodiment, the cell is a human cell. In some embodiments, a method of inducing immunogenic cell death in a cell involves contacting a cell with an mRNA described herein, e.g., an mRNA encoding a polypeptide that induces immunogenic cell death, such as necroptosis or pyroptosis. In certain embodiments, such a method involves contacting a cell with an isolated mRNA encoding the polypeptide that induces immunogenic cell death. In particular embodiments, the cell is contacted with a lipid nanoparticle composition including an mRNA encoding a polypeptide that induces immunogenic cell death. Upon contacting the cell with the lipid nanoparticle composition or the isolated mRNA, the mRNA may be taken up and translated in the cell to produce the polypeptide that induces immunogenic cell death. In one embodiment, the immunogenic cell death is characterized by cell swelling, plasma membrane rupture and release of cytosolic contents of the cell. In one embodiment, the immunogenic cell death is characterized by release of ATP and HMGB1 from the cell.

The invention further provides methods of selectively inducing immunogenic cell death in a cancer cell as compared to a normal cell. In some embodiments, a method of selectively inducing immunogenic cell death in a cancer cell involves contacting a cell with an mRNA described herein, e.g., an mRNA encoding a polypeptide that induces immunogenic cell death, wherein the mRNA further comprises a regulatory element that reduces expression of the polypeptide in normal cells as compared to cancer cells. In particular embodiments, the regulatory element is a binding site for a microRNA that has greater expression in normal cells than cancer cells (e.g., a miR-122 binding site), wherein binding of the microRNA to the binding site inhibits expression of the polypeptide. In particular embodiments, the cell is contacted with a nanoparticle composition comprising an mRNA comprising a region encoding the polypeptide and a microRNA binding site. Upon contacting the cell with the nanoparticle composition or the isolated mRNA, the mRNA may be taken up and translated in the cell to produce the polypeptide. Expression of the polypeptide is greater in cancer cells than normal cells, resulting in greater induction of immunogenic cell death of cancer cells than normal cells.

In general, the step of contacting a mammalian cell with a composition (e.g., an isolated mRNA, nanoparticle, or pharmaceutical composition of the invention) may be performed in vivo, ex vivo, in culture, or in vitro. In exemplary embodiments of the invention, the step of contacting a mammalian cell with a composition (e.g., an isolated mRNA, nanoparticle, or pharmaceutical composition of the invention) is performed in vivo or ex vivo. The amount of the composition contacted with a cell, and/or the amount of mRNA therein, may depend on the type of cell or tissue being contacted, the means of administration, the physiochemical characteristics of the composition and the mRNA (e.g., size, charge, and chemical composition) therein, and other factors. In general, an effective amount of the composition will allow for efficient production of the encoded polypeptide in the cell. Metrics for efficiency may include polypeptide translation (indicated by polypeptide expression), level of mRNA degradation, and immune response indicators.

The step of contacting a composition including an mRNA, or an isolated mRNA, with a cell may involve or cause transfection. In some embodiments, a phospholipid included in a lipid nanoparticle may facilitate transfection and/or increase transfection efficiency, for example, by interacting and/or fusing with a cellular or intracellular membrane. Transfection may allow for the translation of the mRNA within the cell. The ability of a composition of the invention (e.g., a lipid nanoparticle or isolated mRNA) to induce immunogenic cell death may be readily determined, for example by comparing the ability of the composition to induce immunogenic cell death as compared to known agents or manipulations that may induce immunogenic cell death, including but not limited to: engagement of TNFR, TLR or TCR receptors, DNA damage or viral infection. A variety of methods of determining whether an agent can induce immunogenic cell death are known in the art, for example, stains and dyes (e.g., CELLTOX™, MITOTRACKER® Red, propidium iodide, and YOYO3), cell viability assays, and assays (e.g., ELISAs) detecting release of DAMPs (“damage associated molecular patterns”), including release of ATP, HMGB1, IL-1a, uric acid, DNA fragments and/or mitochondrial contents.

### Prophylactic and Therapeutic Methods

The methods of the disclosure for enhancing an immune response to an antigen(s) of interest in a subject can be used in a variety of clinical, prophylactic or therapeutic applications. For example, the methods can be used to stimulate anti-tumor immunity in a subject with a tumor or in a subject at risk of a tumor (e.g., potentially exposed to an oncogenic virus, such as HPV). Furthermore, the methods can be used to stimulate anti-pathogen immunity in a subject, such as to treat a subject suffering from a pathogenic infection or to provide protective immunity to the subject against the pathogen (e.g., vaccination against the pathogen) prior to exposure to the pathogen.

Accordingly, in one aspect, the disclosure pertains to a method of stimulating an immunogenic response to a tumor or tumor antigen in a subject in need thereof, the method comprising administering to the subject a composition of the disclosure (or lipid nanoparticle thereof, or pharmaceutical composition thereof) comprising at least one mRNA construct encoding:(i) at least one tumor antigen of interest and (ii) a polypeptide that enhances an immune response against the tumor antigen(s) of interest, such that an immune response to the tumor antigen(s) of interest is enhanced. Suitable tumor antigens of interest include those described herein (e.g. tumor neoantigens, including mutant KRAS antigens; oncogenic viral antigens, including HPV antigens). In one embodiment of the method, the subject is administered a mutant KRAS antigen-STING mRNA construct encoding a sequence shown in any of SEQ ID NOs: 107-130.

The disclosure also provides methods of treating or preventing a cancer in a subject in need thereof that involve providing or administering at least one mRNA composition described herein (i.e., an immune potentiator mRNA and an antigen-encoding mRNA, in the same or separate mRNA constructs) to the subject. In related embodiments, the subject is provided with or administered a nanoparticle (e.g., a lipid nanoparticle) comprising the mRNA(s). In further related embodiments, the subject is provided with or administered a pharmaceutical composition of the disclosure to the subject. In particular embodiments, the pharmaceutical composition comprises an mRNA(s) encoding an antigen and an immunostimulatory polypeptide as described herein, or it comprises a nanoparticle comprising the mRNA(s). In particular embodiments, the mRNA(s) is present in a nanoparticle, e.g., a lipid nanoparticle. In particular embodiments, the mRNA(s) or nanoparticle is present in a pharmaceutical composition.

In certain embodiments, the subject in need thereof has been diagnosed with a cancer, or is considered to be at risk of developing a cancer. In some embodiments, the cancer is liver cancer, colorectal cancer, a melanoma cancer, a pancreatic cancer, a NSCLC, a cervical cancer or a head or neck cancer. In particular embodiments, the liver cancer is hepatocellular carcinoma. In some embodiments, the colorectal cancer is a primary tumor or a metastasis. In some embodiments, the cancer is a hematopoetic cancer. In some embodiments, the cancer is an acute myeloid leukemia, a chronic myeloid leukemia, a chronic myelomonocytic leukemia, a myelodystrophic syndrome (including refractory anemias and refractory cytopenias) or a myeloproliferative neoplasm or disease (including polycythemia vera, essential thrombocytosis and primary myelofibrosis). In other embodiments, the cancer is a blood-based cancer or a hematopoetic cancer. In yet other embodiments, the cancer is an HPV-associated cancer, such as cervical, penile, vaginal, vulval, anal and/or oropharyngeal cancer.

Selectivity for a particular cancer type can be achieved through the combination of use of an appropriate LNP formulation (e.g., targeting specific cell types) in combination with appropriate regulatory site(s) (e.g., microRNAs) engineered into the mRNA constructs.

In some embodiments, the mRNA(s), nanoparticle, or pharmaceutical composition is administered to the patient parenterally. In particular embodiments, the subject is a mammal, e.g., a human. In various embodiments, the subject is provided with an effective amount of the mRNA(s).

The methods of treating cancer can further include treatment of the subject with additional agents that enhance an anti-tumor response in the subject and/or that are cytotoxic to the tumor (e.g., chemotherapeutic agents). Suitable therapeutic agents for use in combination therapy include small molecule chemotherapeutic agents, including protein tyrosine kinase inhibitors, as well as biological anti-cancer agents, such as anti-cancer antibodies, including but not limited to those discussed further below. Combination therapy can include administering to the subject an immune checkpoint inhibitor to enhance anti-tumor immunity, such as PD-1 inhibitors, PD-L1 inhibitors and CTLA-4 inhibitors. Other modulators of immune checkpoints may target OX-40, OX-40L or ICOS. In one embodiment, an agent that modulates an immune checkpoint is an antibody. In another embodiment, an agent that modulates an immune checkpoint is a protein or small molecule modulator. In another embodiment, the agent (such as an mRNA) encodes an antibody modulator of an immune checkpoint. Non-limiting examples of immune checkpoint inhibitors that can be used in combination therapy include pembrolizumab, alemtuzumab, nivolumab, pidilizumab, ofatumumab, rituximab, MEDI0680 and PDR001, AMP-224, PF-06801591, BGB-A317, REGN2810, SHR-1210, TSR-042, affimer, avelumab (MSB0010718C), atezolizumab (MPDL3280A), durvalumab (MEDI4736), BMS936559, ipilimumab, tremelimumab, AGEN1884, MEDI6469 and MOXR0916.

In one embodiment, the invention provides a method of preventing or treating an HPV-associated cancer in a subject in need thereof, the method comprising administering to the subject a composition of the disclosure (or lipid nanoparticle thereof, or pharmaceutical composition thereof) comprising at least one mRNA construct encoding:(i) at least one HPV antigen of interest and (ii) a polypeptide that enhances an immune response against the HPV antigen(s) of interest, such that an immune response to the HPV antigen(s) of interest is enhanced. In various embodiments, the HPV-associated cancer is cervical, penile, vaginal, vulval, anal and/or oropharyngeal cancer. In certain embodiments, the HPV antigen(s) encoded by the mRNA construct(s) is at least one E6 antigen, at least one E7 antigen or both at least one E6 antigen and at least one E7 antigen. In one embodiment, the E6 antigen(s) and/or the E7 antigen(s) are soluble. In another embodiment, the E6 antigen(s) and/or the E7 antigen(s) are intracellular. In one embodiment, the polypeptide that enhances an immune response against the HPV antigen(s) of interest is a STING polypeptide (e.g., a constitutively active STING polypeptide). In one embodiment, the HPV antigen(s) and the STING polypeptide are encoded on different mRNAs and are coformulated in a lipid nanoparticle prior to coadministration to the subject. In another embodiment, the HPV antigen(s) and the STING polypeptide are encoded on the same mRNA. In one embodiment, the composition encoding the HPV antigen(s) and the immune potentiator is administered to a subject at risk of exposure to HPV, to thereby provide prophylactic protection against HPV infection and development of an HPV-associated cancer(s). In another embodiment, the composition encoding the HPV antigen(s) and the immune potentiator is administered to a subject infected with HPV and/or having an HPV-associated cancer, to thereby provide therapeutic activity against HPV by enhancing an immune response against HPV in the subject. In certain embodiments, a subject with an HPV-associated cancer is also treated with an immune checkpoint inhibitor (e.g., anti-CTLA-4, anti-PD-1, anti-PD-L1 or the like), in combination with the treatment with the HPV+immune potentiator vaccine.

In another aspect, the disclosure pertains to a method of stimulating an immunogenic response to a pathogen in a subject in need thereof, the method comprising administering to the subject a composition of the disclosure (or lipid nanoparticle thereof, or pharmaceutical composition thereof) comprising at least one mRNA construct encoding:(i) at least one pathogen antigen of interest and (ii) a polypeptide that enhances an immune response against the pathogen antigen(s) of interest, such that an immune response to the pathogen antigen(s) of interest is enhanced. In one embodiment, the at least one pathogen antigen is from a pathogen selected from the group consisting of viruses, bacteria, protozoa, fungi and parasites.

Suitable pathogen antigens of interest include those described herein. In one embodiment, the pathogen antigen(s) is a viral antigen(s). In one embodiment, the pathogen antigen(s) is a human papillomavirus (HPV) antigen, such as an E6 antigen (e.g., comprising an amino acid sequence as shown in any of SEQ ID NOs: 36-72) or a E7 antigen (e.g. comprising an amino acid sequence as shown in any of SEQ ID NOs: 73-94). In one embodiment, the pathogen antigen(s) is a bacterial antigen(s), such as a multivalent bacterial antigen.

In one embodiment of the method of stimulating an immunogenic response to a pathogen antigen(s) in a subject in need thereof, the mRNA construct(s), lipid nanoparticle or pharmaceutical composition is administered to the subject parenterally. In one embodiment, the mRNA(s), lipid nanoparticle or pharmaceutical composition is administered by once weekly infusion.

A pharmaceutical composition including one or more mRNAs of the disclosure may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. In some embodiments, a composition is administered intramuscularly. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In particular embodiments, a dose of about 0.005 mg/kg to about 5 mg/kg of mRNA or nanoparticle of the disclosure may be administrated.

In some embodiments, a composition of the disclosure comprising both an immune potentiator mRNA construct (e.g., STING construct) and an antigen construct (e.g., vaccine construct) is formulated such that it is optimized as a function of a fixed dosage of the immune potentiator construct. Non-limiting examples of a fixed dosage of the immune potentiator construct include 0.001 mg/kg, 0.005 mg/kg, 0.01 mg/kg, 0.05 mg/kg, 0.1 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 0.0001 mg/kg to 10 mg/kg, 0.001 mg/kg to 10 mg/kg, 0.005 mg/kg to 10 mg/kg, 0.01 mg/kg to 10 mg/kg, 0.1 mg/kg to 10 mg/kg, 1 mg/kg to 10 mg/kg, 2 mg/kg to 10 mg/kg, 5 mg/kg to 10 mg/kg, 0.0001 mg/kg to 5 mg/kg, 0.001 mg/kg to 5 mg/kg, 0.005 mg/kg to 5 mg/kg, 0.01 mg/kg to 5 mg/kg, 0.1 mg/kg to 10 mg/kg, 1 mg/kg to 5 mg/kg, 2 mg/kg to 5 mg/kg, 0.0001 mg/kg to 1 mg/kg, 0.001 mg/kg to 1 mg/kg, 0.005 mg/kg to 1 mg/kg, 0.01 mg/kg to 1 mg/kg, or 0.1 mg/kg to 1 mg/kg in a given dose, where a dose of 1 mg/kg provides 1 mg of mRNA per 1 kg of subject body weight.

In another embodiment, a composition of the disclosure comprising both an immune potentiator mRNA construct (e.g., STING construct) and an antigen construct (e.g., vaccine construct) is formulated such that it is optimized as a function of a fixed dosage of the antigen construct. Non-limiting examples of a fixed dosage of the antigen construct include 0.001 mg/kg, 0.005 mg/kg, 0.01 mg/kg, 0.05 mg/kg, 0.1 mg/kg, 1 mg/kg, 2 mg/kg, 3 mg/kg, 4 mg/kg, 5 mg/kg, 6 mg/kg, 7 mg/kg, 8 mg/kg, 9 mg/kg, 10 mg/kg, 0.0001 mg/kg to 10 mg/kg, 0.001 mg/kg to 10 mg/kg, 0.005 mg/kg to 10 mg/kg, 0.01 mg/kg to 10 mg/kg, 0.1 mg/kg to 10 mg/kg, 1 mg/kg to 10 mg/kg, 2 mg/kg to 10 mg/kg, 5 mg/kg to 10 mg/kg, 0.0001 mg/kg to 5 mg/kg, 0.001 mg/kg to 5 mg/kg, 0.005 mg/kg to 5 mg/kg, 0.01 mg/kg to 5 mg/kg, 0.1 mg/kg to 10 mg/kg, 1 mg/kg to 5 mg/kg, 2 mg/kg to 5 mg/kg, 0.0001 mg/kg to 1 mg/kg, 0.001 mg/kg to 1 mg/kg, 0.005 mg/kg to 1 mg/kg, 0.01 mg/kg to 1 mg/kg, or 0.1 mg/kg to 1 mg/kg in a given dose, where a dose of 1 mg/kg provides 1 mg of mRNA per 1 kg of subject body weight.

In some embodiments the dosage of the RNA polynucleotide (immune potentiator RNA polynucleotide, antigen-encoding RNA polynucleotide, or both) in the immunomodulatory therapeutic composition is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 100-300 μg, 120-250 μg, 150-250 μg, 180-280 μg, 200-300 μg, 30-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the immunomodulatory therapeutic composition is administered to the subject by intradermal or intramuscular injection. In some embodiments, the immunomodulatory therapeutic composition is administered to the subject on day zero. In some embodiments, a second dose of the immunomodulatory therapeutic composition is administered to the subject on day seven, or day fourteen or day twenty one.

In some embodiments, a dosage of 25 micrograms of the RNA polynucleotide is included in the immunomodulatory therapeutic composition administered to the subject. In some embodiments, a dosage of 10 micrograms of the RNA polynucleotide is included in the immunomodulatory therapeutic composition administered to the subject. In some embodiments, a dosage of 30 micrograms of the RNA polynucleotide is included in the immunomodulatory therapeutic composition administered to the subject. In some embodiments, a dosage of 100 micrograms of the RNA polynucleotide is included in the immunomodulatory therapeutic composition administered to the subject. In some embodiments, a dosage of 50 micrograms of the RNA polynucleotide is included in the immunomodulatory therapeutic composition administered to the subject. In some embodiments, a dosage of 75 micrograms of the RNA polynucleotide is included in the immunomodulatory therapeutic composition administered to the subject. In some embodiments, a dosage of 150 micrograms of the RNA polynucleotide is included in the immunomodulatory therapeutic composition administered to the subject. In some embodiments, a dosage of 400 micrograms of the RNA polynucleotide is included in the immunomodulatory therapeutic composition administered to the subject. In some embodiments, a dosage of 300 micrograms of the RNA polynucleotide is included in the immunomodulatory therapeutic composition administered to the subject. In some embodiments, a dosage of 200 micrograms of the RNA polynucleotide is included in the immunomodulatory therapeutic composition administered to the subject. In some embodiments, the RNA polynucleotide accumulates at a 100 fold higher level in the local lymph node in comparison with the distal lymph node. In other embodiments the immunomodulatory therapeutic composition is chemically modified and in other embodiments the immunomodulatory therapeutic composition is not chemically modified.

In some embodiments, the effective amount is a total dose of 1-100 μg. In some embodiments, the effective amount is a total dose of 100 μg. In some embodiments, the effective amount is a dose of 25 μg administered to the subject a total of one or two times. In some embodiments, the effective amount is a dose of 100 μg administered to the subject a total of two times. In some embodiments, the effective amount is a dose of 1 μg-10 μg, 1 μg-20 μg, 1 μg-30 μg, 5 μg-10 μg, 5 μg-20 μg, 5 μg-30 μg, 5 μg-40 μg, 5 μg-50 μg, 10 μg-15 μg, 10 μg-20 μg, 10 μg-25 μg, 10 μg-30 μg, 10 μg-40 μg, 10 μg-50 μg, 10 μg-60 μg, 15 μg-20 μg, 15 μg-25 μg, 15 μg-30 μg, 15 μg-40 μg, 15 μg-50 μg, 20 μg-25 μg, 20 μg-30 μg, 20 μg-40 μg 20 μg-50 μg, 20 μg-60 μg, 20 μg-70 μg, 20 μg-75 μg, 30 μg-35 μg, 30 μg-40 μg, 30 μg-45 μg 30 μg-50 μg, 30 μg-60 μg, 30 μg-70 μg, 30 μg-75 μg which may be administered to the subject a total of one or two times or more.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). For example, in certain embodiments, a composition of the disclosure comprising both an immune potentiator mRNA construct (e.g., STING construct) and an antigen construct (e.g., vaccine construct) is administered at least two times wherein the second dose is administered at least one day, or at least 3 days, or least 7 days, or at least 10 days, or at least 14 days, or at least 21 days, or at least 28 days, or at least 35 days, or at least 42 days or at least 48 days after the first dose is administered. In certain embodiments, a first and second dose are administered on days 0 and 2, respectively, or on days 0 and 7 respectively, or on days 0 and 14, respectively, or on days 0 and 21, respectively, or on days 0 and 48, respectively. Additional doses (i.e., third doses, fourth doses, etc.) can be administered on the same or a different schedule on which the first two doses were administered. For example, in some embodiments, the first and second dosages are administered 7 days apart and then one or more additional doses are administered weekly thereafter. In another embodiment, the first and second dosages are administered 7 days apart and then one or more additional doses are administered every two weeks thereafter.

In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, a pharmaceutical composition of the disclosure may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the disclosure, or imaging, diagnostic, or prophylactic compositions thereof in combination with agents that improve their bioavailability, reduce and/or modify their metabolism, inhibit their excretion, and/or modify their distribution within the body.

Exemplary therapeutic agents that may be administered in combination with the compositions of the disclosure include, but are not limited to, cytotoxic, chemotherapeutic, and other therapeutic agents. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

Immune checkpoint inhibitors such as pembrolizumab or nivolumab, which target the interaction between programmed death receptor 1/programmed death ligand 1 (PD-1/PD-L1) and PD-L2, have been recently approved for the treatment of various malignancies and are currently being investigated in clinical trials for various cancers including melanoma, head and neck squamous cell carcinoma (HNSCC).

Accordingly, one aspect of the disclosure relates to combination therapy in which a subject is previously treated with a PD-1 antagonist prior to administration of a lipid nanoparticle or composition of the present disclosure. In another aspect, the subject has been treated with a monoclonal antibody that binds to PD-1 prior to administration of a lipid nanoparticle or composition of the present disclosure. In another aspect, the subject has been administered a lipid nanoparticle or composition of the disclosure prior to treatment with an anti-PD-1 monoclonal antibody therapy. In some aspects, the anti-PD-1 monoclonal antibody therapy comprises nivolumab, pembrolizumab, pidilizumab, or any combination thereof.

In another aspect, the subject has been treated with a monoclonal antibody that binds to PD-L1 prior to administration of a lipid nanoparticle or composition of the present disclosure. In another aspect, the subject is administered a lipid nanoparticle or composition prior to treatment with an anti-PD-L1 monoclonal antibody therapy. In some aspects, the anti-PD-L1 monoclonal antibody therapy comprises durvalumab, avelumab, MEDI473, BMS-936559, aezolizumab, or any combination thereof.

In some aspects, the subject has been treated with a CTLA-4 antagonist prior to treatment with the compositions of present disclosure. In another aspect, the subject has been previously treated with a monoclonal antibody that binds to CTLA-4 prior to administration of a lipid nanoparticle or composition of the present disclosure. In some aspects, the subject has been administered a lipid nanoparticle or composition prior to treatment with an anti-CTLA-4 monoclonal antibody. In some aspects, the anti-CTLA-4 antibody therapy comprises ipilimumab or tremelimumab.

In any of the foregoing or related aspects, the disclosure provides a lipid nanoparticle, and an optional pharmaceutically acceptable carrier, or a pharmaceutical composition for use in treating or delaying progression of cancer in an individual, wherein the treatment comprises administration of the composition in combination with a second composition, wherein the second composition comprises a checkpoint inhibitor polypeptide and an optional pharmaceutically acceptable carrier.

In any of the foregoing or related aspects, the disclosure provides use of a lipid nanoparticle, and an optional pharmaceutically acceptable carrier, in the manufacture of a medicament for treating or delaying progression of cancer in an individual, wherein the medicament comprises the lipid nanoparticle and an optional pharmaceutically acceptable carrier and wherein the treatment comprises administration of the medicament in combination with a composition comprising a checkpoint inhibitor polypeptide and an optional pharmaceutically acceptable carrier.

In any of the foregoing or related aspects, the disclosure provides a kit comprising a container comprising a lipid nanoparticle, and an optional pharmaceutically acceptable carrier, or a pharmaceutical composition, and a package insert comprising instructions for administration of the lipid nanoparticle or pharmaceutical composition for treating or delaying progression of cancer in an individual. In some aspects, the package insert further comprises instructions for administration of the lipid nanoparticle or pharmaceutical composition in combination with a composition comprising a checkpoint inhibitor polypeptide and an optional pharmaceutically acceptable carrier for treating or delaying progression of cancer in an individual.

In any of the foregoing or related aspects, the disclosure provides a kit comprising a medicament comprising a lipid nanoparticle, and an optional pharmaceutically acceptable carrier, or a pharmaceutical composition, and a package insert comprising instructions for administration of the medicament alone or in combination with a composition comprising a checkpoint inhibitor polypeptide and an optional pharmaceutically acceptable carrier for treating or delaying progression of cancer in an individual. In some aspects, the kit further comprises a package insert comprising instructions for administration of the first medicament prior to, current with, or subsequent to administration of the second medicament for treating or delaying progression of cancer in an individual.

In any of the foregoing or related aspects, the disclosure provides a lipid nanoparticle, a composition, or the use thereof, or a kit comprising a lipid nanoparticle or a composition as described herein, wherein the checkpoint inhibitor polypeptide inhibits PD1, PD-L1, CTLA4, or a combination thereof. In some aspects, the checkpoint inhibitor polypeptide is an antibody. In some aspects, the checkpoint inhibitor polypeptide is an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, and a combination thereof. In some aspects, the checkpoint inhibitor polypeptide is an anti-PD-L1 antibody selected from atezolizumab, avelumab, or durvalumab. In some aspects, the checkpoint inhibitor polypeptide is an anti-CTLA-4 antibody selected from tremelimumab or ipilimumab. In some aspects, the checkpoint inhibitor polypeptide is an anti-PD1 antibody selected from nivolumab or pembrolizumab.

In related aspects, the disclosure provides a method of reducing or decreasing a size of a tumor or inhibiting a tumor growth in a subject in need thereof comprising administering to the subject any of the foregoing or related lipid nanoparticles of the disclosure, or any of the foregoing or related compositions of the disclosure.

In related aspects, the disclosure provides a method inducing an anti-tumor response in a subject with cancer comprising administering to the subject any of the foregoing or related lipid nanoparticles of the disclosure, or any of the foregoing or related compositions of the disclosure. In some aspects, the anti-tumor response comprises a T-cell response. In some aspects, the T-cell response comprises CD8+ T cells.

In some aspects of the foregoing methods, the method further comprises administering a second composition comprising a checkpoint inhibitor polypeptide, and an optional pharmaceutically acceptable carrier. In some aspects, the checkpoint inhibitor polypeptide inhibits PD1, PD-L1, CTLA4, or a combination thereof. In some aspects, the checkpoint inhibitor polypeptide is an antibody. In some aspects, the checkpoint inhibitor polypeptide is an antibody selected from an anti-CTLA4 antibody or antigen-binding fragment thereof that specifically binds CTLA4, an anti-PD1 antibody or antigen-binding fragment thereof that specifically binds PD1, an anti-PD-L1 antibody or antigen-binding fragment thereof that specifically binds PD-L1, and a combination thereof. In some aspects, the checkpoint inhibitor polypeptide is an anti-PD-L1 antibody selected from atezolizumab, avelumab, or durvalumab. In some aspects, the checkpoint inhibitor polypeptide is an anti-CTLA-4 antibody selected from tremelimumab or ipilimumab. In some aspects, the checkpoint inhibitor polypeptide is an anti-PD1 antibody selected from nivolumab or pembrolizumab.

In some aspects of any of the foregoing or related methods, the composition comprising the checkpoint inhibitor polypeptide is administered by intravenous injection. In some aspects, the composition comprising the checkpoint inhibitor polypeptide is administered once every 2 to 3 weeks. In some aspects, the composition comprising the checkpoint inhibitor polypeptide is administered once every 2 weeks or once every 3 weeks. In some aspects, the composition comprising the checkpoint inhibitor polypeptide is administered prior to, concurrent with, or subsequent to administration of the lipid nanoparticle or pharmaceutical composition thereof.

In any of the foregoing or related aspects, the disclosure provides pharmaceutical composition comprising the lipid nanoparticle, and a pharmaceutically acceptable carrier. In some aspects, the pharmaceutical composition is formulated for intramuscular delivery.

### Therapeutic Methods for Inducing Immunogenic Cell Death

The invention provides a method of stimulating an immunogenic response to a tumor in a subject in need thereof, e.g., a human subject. In one embodiment, the method comprises administering to the subject an effective amount of an mRNA of the invention encoding a polypeptide that induces immunogenic cell death such that an immunogenic response to the tumor is stimulated in the subject. In another embodiment, the method comprises administering to the subject an effective amount of a lipid nanoparticle of the invention comprising an mRNA encoding a polypeptide that induces immunogenic cell death such that an immunogenic response to the tumor is stimulated in the subject. In yet another embodiment, the method comprises administering to the subject a pharmaceutical composition of the invention (e.g., comprising an mRNA or lipid nanoparticle of the invention) such that an immunogenic response to the tumor is stimulated in the subject.

In various embodiments, the method can comprise administering to the subject one or more additional agents that stimulate an inflammatory and/or immune reaction and/or regulate immunoresponsiveness to thereby further promote or enhance an immunogenic response to the tumor in the subject. Suitable types of agents for use as additional agents are described above. In one embodiment, the subject is administered one additional agent. In another embodiment, the subject is administered two additional agents, which additional agents differ from each other. In yet another embodiment, the subject is administered three additional agents, which additional agents differ from each other.

In one embodiment, the method further comprises administering to the subject at least one agent that potentiates an immune response, for example, induces adaptive immunity (e.g., by stimulating Type I interferon production), stimulates an inflammatory response, stimulates NFkB signaling and/or stimulates dendritic cell (DC) mobilization. In one embodiment, the method further comprises administering to the subject at least one agent that induces adaptive immunity. In one embodiment, the agent that induces adaptive immunity is Type I interferon (e.g., a pharmaceutical composition comprising Type I interferon). In another embodiment, the agent that induces adaptive immunity stimulates Type I interferon. Non-limiting examples of agents (e.g., mRNA constructs) that stimulate adaptive immunity include STING, IRF1, IRF3, IRF5, IRF6, IRF7 and IRF8. In another embodiment, the agent stimulates an inflammatory response. Non-limiting examples of agents (e.g., mRNA constructs) that stimulate an inflammatory response include STAT1, STAT2, STAT4, STAT6, NFAT and C/EBPb. In another embodiment, the agent stimulates NFκB signaling. Non-limiting examples of agents (e.g. mRNA constructs) that stimulate NFκB signaling include IKKβ, c-FLIP, RIPK1, IL-27, ApoF and PLP. In another embodiment, the agent stimulates DC mobilization. A non-limiting example of an agent that stimulates DC mobilization is FLT3. In yet another embodiment, the agent that potentiates immune responses is DIABLO (SMAC/DIABLO) (e.g, a DIABLO mRNA construct).

In another embodiment, the method further comprises administering to the subject at least one agent that induces T cell activation or priming. In one embodiment, the agent that induces T cell activation or priming is a cytokine or chemokine. Non-limiting examples of cytokines or chemokines that induce T cell activation or priming include IL-12, IL36g, CCL2, CCL4, CCL20 and CCL21. In one embodiment, the agent that induces T cell activation or priming is a pharmaceutical composition comprising IL-12, IL36g, CCL2, CCL4, CCL20 or CCL21. In another embodiment, the agent that induces T cell activation or priming is an agent (e.g., mRNA construct) that encodes IL-12, IL36g, CCL2, CCL4, CCL20 or CCL21. In yet another embodiment, the agent is an mRNA construct encoding a polypeptide that induces the chemokine or cytokine (e.g., induces IL-12, IL36g, CCL2, CCL4, CCL20 or CCL21).

In another embodiment, the method further comprises administering to the subject at least one agent that modulates an immune checkpoint. In one embodiment, the agent that modulates an immune checkpoint is an antibody. In another embodiment, the agent that modulates an immune checkpoint is an agent (e.g., mRNA construct) that encodes an antibody. In one embodiment, the agent that modulates an immune checkpoint is a CTLA-4 inhibitor, non-limiting examples of which include ipilimumab, tremelimumab and AGEN1884. In another embodiment, the agent that modulates an immune checkpoint is a PD-1 inhibitor, non-limiting examples of which include pembrolizumab, alemtuzumab, atezolizumab, nivolumab, ipilimumab, pidilizumab, ofatumumab, rituximab, MEDI0680 and PDR001, AMP-224, PF-06801591, BGB-A317, REGN2810, SHR-1210, TSR-042, avelumab, durvalumab and affimer. In another embodiment, the agent that modulates an immune checkpoint is a PD-L1 inhibitor, non-limiting examples of which include atezolizumab, avelumab, durvalumab and BMS936559. In yet another embodiment, the agent that modulates an immune checkpoint modulates the activity of OX-40 or OX-40L, non-limiting examples of which include Fc-OX-40L, MEDI6469 (agonist anti-OX40 antibody) and MOXR0916 (agonist anti-OX40 antibody). In yet another embodiment, the agent that modulates an immune checkpoint modulates the activity of ICOS (e.g., ICOS pathway agonists).

In one embodiment, in addition to administering the mRNA encoding a polypeptide that induces immunogenic cell death, the method further comprises administering: (i) at least one agent that potentiates an immune response (e.g., induces induces adaptive immunity, stimulates Type I interferon, stimulates an inflammatory response, stimulates NFκB signaling and/or stimulates DC mobilization); and (ii) at least one agent that induces T cell activation or priming. In another embodiment, the method further comprises administering: (i) at least one agent that potentiates an immune response (e.g., induces adaptive immunity, stimulates Type I interferon, stimulates an inflammatory response, stimulates NFκB signaling and/or stimulates DC mobilization); and (ii) at least one agent that modulates an immune checkpoint. In another embodiment, the method further comprises administering: (i) at least one agent that induces T cell activation or priming; and (ii) at least one agent that modulates an immune checkpoint. In yet another embodiment, the method further comprises administering to the subject: (i) at least one agent that potentiates an immune response (e.g., induces adaptive immunity, stimulates Type I interferon, stimulates an inflammatory response, stimulates NFκB signaling and/or stimulates DC mobilization); (ii) at least one agent that induces T cell activation or priming; and (iii) at least one agent that modulates an immune checkpoint.

In one embodiment of the method of stimulating an immunogenic response to a tumor in a subject in need thereof, the mRNA construct, lipid nanoparticle or pharmaceutical composition is administered to the subject parenterally. In one embodiment, the mRNA, lipid nanoparticle or pharmaceutical composition is administered by once weekly infusion. In one embodiment, the tumor is a liver cancer, a colorectal cancer or a melanoma cancer cell.

In another aspect, the invention provides a method for stimulating an immunogenic response to a tumor in a subject in need thereof, the method comprising administering to the subject an effective amount of:
(i) a first chemically modified messenger RNA (mmRNA) encoding a polypeptide that induces immunogenic cell death, wherein said first mmRNA comprises one or more modified nucleobases;
and at least one of:
(ii) a second mmRNA encoding a polypeptide that potentiates an immune response (e.g., induces adaptive immunity, stimulates Type I interferon, stimulates an inflammatory response, stimulates NFκB signaling and/or stimulates DC mobilization); wherein said second mmRNA comprises one or more modified nucleobases;
(iii) a third mmRNA encoding a polypeptide that induces T cell activation or priming, wherein said third mmRNA comprises one or more modified nucleobases; and/or
(iv) a fourth mmRNA encoding a polypeptide that modulates an immune checkpoint, wherein said fourth mmRNA comprises one or more modified nucleobases, such that an immunogenic response to the tumor is generated in the subject.

The first mmRNA, second mmRNA, third mmRNA and/or fourth mmRNA may be present in the same pharmaceutical composition or lipid nanoparticle that is administered to the subject. Alternatively, the first mmRNA, second mmRNA, third mmRNA and/or fourth mmRNA may be present in different pharmaceutical compositions or lipid nanoparticles that are administered to the subject.

In one embodiment, the first mmRNA and second mmRNA are administered to the subject. In another embodiment, the first mmRNA and third mmRNA are administered to the subject. In another embodiment, the first mmRNA and fourth mmRNA are administered to the subject. In another embodiment, the first mmRNA, second mmRNA and third mmRNA are administered to the subject. In another embodiment, the first mmRNA, second mmRNA and fourth mmRNA are administered to the subject. In another embodiment, the first mmRNA, third mmRNA and fourth mmRNA are administered to the subject. In another embodiment, the first mmRNA, second, third mmRNA and fourth mmRNA are administered to the subject.

In one embodiment, the polypeptide encoded by the first mmRNA is selected from the group consisting of MLKL, RIPK3, RIPK1, DIABLO, FADD, GSDMD, caspase-4, caspase-5, caspase-11, NLRP3, ASC/CARD and Pyrin. In one embodiment, the polypeptide encoded by the second mmRNA is selected from the group consisting of DIABLO, STING, IRF1, IRF3, IRF5, IRF6, IRF7, IRF8, STAT1, STAT2, STAT4, STAT6, NFAT, C/EBPb, IKKβ, c-FLIP, RIPK1, IL-27, ApoF, PLP and FLT3. In one embodiment, the polypeptide encoded by the second mmRNA is selected from the group consisting of DIABLO, STING, IRF3, IRF7, STAT6, IKKβ, c-FLIP and RIPK1. In one embodiment, the polypeptide encoded by the third mmRNA is selected from the group consisting of IL-12, IL36g, CCL2, CCL4, CCL20 and CCL21. In one embodiment, the polypeptide encoded by the fourth mmRNA is selected from the group consisting of PD-1 inhibitors, PD-L1 inhibitors, CTLA-4 inhibitors, OX-40 agonists, OX-40L and ICOS pathway agonists.

The invention also provides methods of treating or preventing a cancer in a subject in need thereof that involve providing or administering an mRNA encoding a polypeptide described herein to the subject. In related embodiments, the subject is provided with or administered a nanoparticle (e.g., a lipid nanoparticle) comprising the mRNA. In further related embodiments, the subject is provided with or administered a pharmaceutical composition of the invention to the subject. In particular embodiments, the pharmaceutical composition comprises an mRNA encoding a polypeptide described herein, or it comprises a nanoparticle comprising the mRNA. In particular embodiments, the mRNA is present in a nanoparticle, e.g., a lipid nanoparticle. In particular embodiments, the mRNA or nanoparticle is present in a pharmaceutical composition. In certain embodiments, the subject in need thereof has been diagnosed with a cancer, or is considered to be at risk of developing a cancer. In some embodiments, the cancer is liver cancer, colorectal cancer or a melanoma cancer. In particular embodiments, the liver cancer is hepatocellular carcinoma. In some embodiments, the colorectal cancer is a primary tumor or a metastasis. In some embodiments, the cancer is a hematopoetic cancer. In some embodiments, the cancer is an acute myeloid leukemia, a chronic myeloid leukemia, a chronic myelomonocytic leukemia, a myelodystrophic syndrome (including refractory anemias and refractory cytopenias) or a myeloproliferative neoplasm or disease (including polycythemia vera, essential thrombocytosis and primary myelofibrosis). In other embodiments, the cancer is a blood-based cancer or a hematopoetic cancer. Selectivity for a particular cancer type can be achieved through the combination of use of an appropriate LNP formulation (e.g., targeting specific cell types) in combination with appropriate regulatory site(s) (e.g., microRNAs) engineered into the mRNA constructs.

In some embodiments, the mRNA, nanoparticle, or pharmaceutical composition is administered to the patient parenterally. In particular embodiments, the subject is a mammal, e.g., a human. In various embodiments, the subject is provided with an effective amount of the mRNA.

The invention further provides methods of treating or preventing cancer in a subject in need thereof, comprising providing the subject with an effective amount of an mRNA described herein, e.g., an mRNA encoding a polypeptide that induces immunogenic cell death, wherein the mRNA further comprises a regulatory element that enhances expression of the polypeptide in cancer cells as compared to normal cells. In particular embodiments, the regulatory element is a binding site for a microRNA that has greater expression in normal cells than cancer cells (e.g., a miR-122 binding site), wherein binding of the microRNA to the binding site inhibits expression of the polypeptide. In particular embodiments, the mRNA is present in a nanoparticle, e.g., a lipid nanoparticle. In particular embodiments, the mRNA or nanoparticle is present in a pharmaceutical composition. The nanoparticle or the isolated mRNA may be taken up and translated in the subject's cells to produce the polypeptide inducing immunogenic cell death. In particular embodiments, expression of the polypeptide is greater in cancer cells than normal cells, resulting in greater immunogenic cell death of cancer cells than normal cells.

In certain embodiments, the present invention includes a method of treating or preventing cancer in a subject in need thereof, comprising providing to the subject a first mRNA described herein, e.g., an mRNA encoding a polypeptide that induces immunogenic cell death, in combination with a therapeutic agent, such as a chemotherapeutic drug or other anti-cancer agent. Suitable therapeutic agents for use in combination therapy include small molecule chemotherapeutic agents, including protein tyrosine kinase inhibitors, as well as biological anti-cancer agents, such as anti-cancer antibodies. Other suitable therapeutic agents for use in combination therapy are described further below.

A pharmaceutical composition including one or more mRNAs of the invention may be administered to a subject by any suitable route. In some embodiments, compositions of the invention are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. However, the present disclosure encompasses the delivery of compositions of the invention by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the invention may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg, from about 2 mg/kg to about 5 mg/kg, from about 0.0001 mg/kg to about 1 mg/kg, from about 0.001 mg/kg to about 1 mg/kg, from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In particular embodiments, a dose of about 0.005 mg/kg to about 5 mg/kg of mRNA or nanoparticle of the invention may be administrated.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations). In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

In some embodiments, a pharmaceutical composition of the invention may be administered in combination with another agent, for example, another therapeutic agent, a prophylactic agent, and/or a diagnostic agent. By “in combination with,” it is not intended to imply that the agents must be administered at the same time and/or formulated for delivery together, although these methods of delivery are within the scope of the present disclosure. For example, one or more compositions including one or more different mRNAs may be administered in combination. Compositions can be administered concurrently with, prior to, or subsequent to, one or more other desired therapeutics or medical procedures. In general, each agent will be administered at a dose and/or on a time schedule determined for that agent. In some embodiments, the present disclosure encompasses the delivery of compositions of the invention, or imaging, diagnostic, or prophylactic compositions thereof in combination with agents that improve their bioavailability, reduce and/or modify their metabolism, inhibit their excretion, and/or modify their distribution within the body.

Exemplary therapeutic agents that may be administered in combination with the compositions of the invention include, but are not limited to, cytotoxic, chemotherapeutic, and other therapeutic agents. Cytotoxic agents may include, for example, taxol, cytochalasin B, gramicidin D, ethidium bromide, emetine, mitomycin, etoposide, teniposide, vincristine, vinblastine, colchicine, doxorubicin, daunorubicin, dihydroxyanthracinedione, mitoxantrone, mithramycin, actinomycin D, 1-dehydrotestosterone, glucocorticoids, procaine, tetracaine, lidocaine, propranolol, puromycin, maytansinoids, rachelmycin, and analogs thereof. Radioactive ions may also be used as therapeutic agents and may include, for example, radioactive iodine, strontium, phosphorous, palladium, cesium, iridium, cobalt, yttrium, samarium, and praseodymium. Other therapeutic agents may include, for example, antimetabolites (e.g., methotrexate, 6-mercaptopurine, 6-thioguanine, cytarabine, and 5-fluorouracil, and decarbazine), alkylating agents (e.g., mechlorethamine, thiotepa, chlorambucil, rachelmycin, melphalan, carmustine, lomustine, cyclophosphamide, busulfan, dibromomannitol, streptozotocin, mitomycin C, and cis-dichlorodiamine platinum (II) (DDP), and cisplatin), anthracyclines (e.g., daunorubicin and doxorubicin), antibiotics (e.g., dactinomycin, bleomycin, mithramycin, and anthramycin), and anti-mitotic agents (e.g., vincristine, vinblastine, taxol, and maytansinoids).

The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, a composition useful for treating cancer may be administered concurrently with a chemotherapeutic agent), or they may achieve different effects (e.g., control of any adverse effects).

### Other Embodiments of the Disclosure

E1. A chemically modified messenger RNA (mmRNA) encoding a polypeptide that induces immunogenic cell death, wherein said mmRNA comprises one or more modified nucleobases.

E2. The mmRNA of embodiment 1, wherein the polypeptide induces necroptosis.

E3. The mmRNA of embodiment 2, wherein the polypeptide is mixed lineage kinase domain-like protein (MLKL), or an immunogenic cell death-inducing fragment thereof.

E4. The mmRNA of embodiment 3, wherein the MLKL polypeptide comprises the amino acid sequence shown in SEQ ID NOs: 1 or 2.

E5. The mmRNA of embodiment 2, wherein the polypeptide is receptor-interacting protein kinase 3 (RIPK3), or an immunogenic cell death-inducing fragment thereof.

E6. The mmRNA of embodiment 5, wherein the RIPK3 polypeptide comprises any of the amino acid sequences shown in SEQ ID NOs: 3-19.

E7. The mmRNA of embodiment 2, wherein the polypeptide is receptor-interacting protein kinase 1 (RIPK1), or an immunogenic cell death-inducing fragment thereof.

E8. The mmRNA of embodiment 7, wherein the RIPK1 polypeptide comprises any of the amino acid sequences shown in SEQ ID NOs: 62-67.

E9. The mmRNA of embodiment 2, wherein the polypeptide is direct IAP binding protein with low pI (DIABLO), or an immunogenic cell death-inducing fragment thereof.

E10. The mmRNA of embodiment 9, wherein the DIABLO polypeptide comprises any of the amino acid sequences shown in SEQ ID NOs: 26-33.

E11. The mmRNA of embodiment 2, wherein the polypeptide is Fas-associated protein with death domain (FADD), or an immunogenic cell death-inducing fragment thereof.

E12. The mmRNA of embodiment 11, wherein the FADD polypeptide comprises any of the amino acid sequences shown in SEQ ID NOs: 56-61.

E13. The mmRNA of embodiment 1, wherein the polypeptide induces pyroptosis.

E14. The mmRNA of embodiment 13, wherein the polypeptide is gasdermin D (GSDMD), or an immunogenic cell death-inducing fragment thereof.

E15. The mmRNA of embodiment 14, wherein the GSDMD polypeptide comprises any of the amino acid sequences shown in SEQ ID NOs: 20-25.

E16. The mmRNA of embodiment 13, wherein the polypeptide is caspase-4, an immunogenic cell death-inducing fragment thereof.

E17. The mmRNA of embodiment 16, wherein the caspase-4 polypeptide comprises any of the amino acid sequences shown in SEQ ID NOs: 34-38.

E18. The mmRNA of embodiment 13, wherein the polypeptide is caspase-5, an immunogenic cell death-inducing fragment thereof.

E19. The mmRNA of embodiment 18, wherein the caspase-5 polypeptide comprises any of the amino acid sequences shown in SEQ ID NOs: 39-43.

E20. The mmRNA of embodiment 13, wherein the polypeptide is caspase-11, an immunogenic cell death-inducing fragment thereof.

E21. The mmRNA of embodiment 20, wherein the caspase-11 polypeptide comprises any of the amino acid sequences shown in SEQ ID NOs: 44-48.

E22. The mmRNA of embodiment 13, wherein the polypeptide is NLRP3, an immunogenic cell death-inducing fragment thereof.

E23. The mmRNA of embodiment 22, wherein the NLRP3 polypeptide comprises any of the amino acid sequences shown in SEQ ID NOs: 51-52.

E24. The mmRNA of embodiment 13, wherein the polypeptide is a Pyrin domain, an immunogenic cell death-inducing fragment thereof.

E25. The mmRNA of embodiment 24, wherein the Pyrin domain polypeptide comprises any of the amino acid sequences shown in SEQ ID NOs: 49-50.

E26. The mmRNA of embodiment 13, wherein the polypeptide is ASC/PYCARD, an immunogenic cell death-inducing fragment thereof.

E27. The mmRNA of embodiment 26, wherein the ASC/PYCARD polypeptide comprises any of the amino acid sequences shown in SEQ ID NOs: 53-54.

E28. The mmRNA of any one of the preceding embodiments wherein the mmRNA comprises a 5′ UTR, a codon optimized open reading frame encoding the polypeptide, a 3′ UTR and a 3′ tailing region of linked nucleosides.

E29. The mmRNA of embodiment 28, wherein the mmRNA further comprises one or more microRNA (miRNA) binding sites.

E30. The mmRNA of any one of the preceding embodiments wherein the mmRNA is fully modified.

E31. The mmRNA of any one of the preceding embodiments wherein the mmRNA comprises pseudouridine (v), pseudouridine (w) and 5-methyl-cytidine (m⁵C), 1-methyl-pseudouridine (m¹ψ), 1-methyl-pseudouridine (m¹ψ) and 5-methyl-cytidine (m⁵C), 2-thiouridine (s²U), 2-thiouridine and 5-methyl-cytidine (m⁵C), 5-methoxy-uridine (mo⁵U), 5-methoxy-uridine (mo⁵U) and 5-methyl-cytidine (m⁵C), 2′-O-methyl uridine, 2′-O-methyl uridine and 5-methyl-cytidine (m⁵C), N6-methyl-adenosine (m⁶A) or N6-methyl-adenosine (m⁶A) and 5-methyl-cytidine (m⁵C).

E32. The mmRNA of any one of the preceding embodiments wherein the mmRNA comprises pseudouridine (w), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine, or combinations thereof.

E33. The mmRNA of any one of the preceding embodiments wherein the mmRNA comprises 1-methyl-pseudouridine (m¹ψ), 5-methoxy-uridine (mo⁵U), 5-methyl-cytidine (m⁵C), pseudouridine (ψ), α-thio-guanosine, or α-thio-adenosine, or combinations thereof.

E34. A lipid nanoparticle comprising the mmRNA of any one of embodiments 1-33.

E35. The lipid nanoparticle of embodiment 34, which is a liposome.

E36. The lipid nanoparticle of embodiment 34, which comprises a cationic and/or ionizable lipid.

E37. The lipid nanoparticle of embodiment 36, wherein the cationic and/or ionizable lipid is 2,2-dilinoleyl-4-methylaminoethyl-[1,3]-dioxolane (DLin-KC2-DMA) or dilinoleyl-methyl-4-dimethylaminobutyrate (DLin-MC3-DMA).

E38. The lipid nanoparticle of any one of embodiments 34-37, wherein the lipid nanoparticle further comprises a targeting moiety conjugated to the outer surface of the lipid nanoparticle.

E39. A pharmaceutical composition comprising the mmRNA of any of embodiments 1-33 or the lipid nanoparticle of any one of embodiments 34-38, and a pharmaceutically acceptable carrier, diluent or excipient.

E40. A method for inducing immunogenic cell death of a cell, the method comprising contacting the cell with the mmRNA of any one of embodiments 1-33, the lipid nanoparticle of any one of embodiments 34-38 or the pharmaceutical composition of embodiment 39 such that immunogenic cell death of the cell occurs.

E41. The method of embodiment 40, wherein immunogenic cell death is characterized by plasma membrane rupture and release of cytosolic contents of the cell.

E42. The method of embodiment 41, wherein ATP and HMGB1 are released from the cell.

E43. The method of any one of embodiments 40-42, wherein the contacting occurs in vitro or in vivo.

E44. The method of any one of embodiments 40-43, wherein the cell is a cancer cell.

E45. The method of embodiment 44, wherein the cancer cell is a liver cancer cell, a colorectal cancer cell or a melanoma cancer cell.

E46. The method of any one of embodiments 40-45, wherein the cell is a human cell. E47. A method of stimulating an immunogenic response to a tumor in a subject in need thereof, the method comprising administering to the subject an effective amount of the mmRNA of any one of embodiments 1-33, the lipid nanoparticle of any one of embodiments 34-38, or the pharmaceutical composition of embodiment 39, such that an immunogenic response to the tumor is stimulated in the subject.

E48. The method of embodiment 47, which further comprises administering to the subject at least one agent that potentiates an immune response, wherein the at least one agent that potentiates an immune response induces adaptive immunity, stimulates Type 1 interferon, stimulates an inflammatory response, stimulates NF□B signaling or stimulates dendritic cell mobilization.

E49. The method of embodiment 48, wherein the at least one agent induces adaptive immunity by stimulating Type 1 interferon.

E50. The method of embodiment 47, which further comprises administering to the subject at least one agent that induces T cell activation or priming.

E51. The method of embodiment 50, wherein the at least one agent that induces T cell activation or priming is a cytokine or chemokine.

E52. The method of embodiment 47, which further comprises administering to the subject at least one agent that modulates an immune checkpoint.

E53. The method of embodiment 47, which further comprises administering to the subject: (i) at least one agent that potentiates an immune response; (ii) at least one agent that induces T cell activation or priming; and (iii) at least one agent that modulates an immune checkpoint.

E54. The method of any one of embodiments 47-53, wherein the mmRNA, lipid nanoparticle or pharmaceutical composition is administered to the subject parenterally.

E55. The method of embodiment 54, wherein the mmRNA, lipid nanoparticle or pharmaceutical composition is administered by once weekly infusion.

E56. The method of any one of embodiments 47-55, wherein the subject is a human.

E57. The method of any one of embodiments 47-56, wherein the tumor is a liver cancer or a colorectal cancer.

E58. A method for stimulating an immunogenic response to a tumor in a subject in need thereof, the method comprising administering to the subject an effective amount of:
(i) a first chemically modified messenger RNA (mmRNA) encoding a polypeptide that induces immunogenic cell death, wherein said first mmRNA comprises one or more modified nucleobases;
and at least one of:
(ii) a second mmRNA encoding a polypeptide that potentiates an immune response, wherein said second mmRNA comprises one or more modified nucleobases;
(iii) a third mmRNA encoding a polypeptide that induces T cell activation or priming, wherein said third mmRNA comprises one or more modified nucleobases; and/or
(iv) a fourth mmRNA encoding a polypeptide that modulates an immune checkpoint, wherein said fourth mmRNA comprises one or more modified nucleobases, such that an immunogenic response to the tumor is generated in the subject.

E59. The method of embodiment 58, wherein the second mmRNA encodes a polypeptide that induces adaptive immunity, stimulates Type 1 interferon, stimulates an inflammatory response, stimulates NF□B signaling or stimulates dendritic cell mobilization.

E60. The method of embodiment 58, wherein the first mmRNA and second mmRNA are administered to the subject.

E61. The method of embodiment 58, wherein the first mmRNA, the second mmRNA and the third mmRNA are administered to the subject.

E62. The method of embodiment 58, wherein the first mmRNA, the second mmRNA, the third mmRNA and the fourth mmRNA are administered to the subject.

E63. The method of any one of embodiments 58-62, wherein the first mmRNA, second mmRNA, third mmRNA and/or fourth mmRNA are present in the same pharmaceutical compositions or lipid nanoparticle which is administered to the subject.

E64. The method of any one of embodiments 58-63, wherein the polypeptide encoded by the first mmRNA is selected from the group consisting of MLKL, RIPK3, RIPK1, DIABLO, FADD, GSDMD, caspase-4, caspase-5, caspase-11, NLRP3, ASC/PYCARD and Pyrin.

E65. The method of any one of embodiments 58-64, wherein the polypeptide encoded by the second mmRNA is selected from the group consisting of DIABLO, STING, IRF1, IRF3, IRF5, IRF6, IRF7, IRF8, STAT1, STAT2, STAT4, STAT6, NFAT, C/EBPb, IKKβ, c-FLIP, RIPK1, IL-27, ApoF, PLP and FLT3.

E66. The method of any one of embodiments 58 and 60-64, wherein the polypeptide encoded by the third mmRNA is selected from the group consisting of IL-12, IL36g, CCL2, CCL4, CCL20 and CCL21.

E67. The method of any one of embodiments 58 and 61-65, wherein the polypeptide encoded by the fourth mmRNA is selected from the group consisting of PD-1 inhibitors, PD-L1 inhibitors, CTLA-4 inhibitors, OX-40 agonists, OX-40L and ICOS pathway agonists. E68. A method for stimulating an immunogenic response to a tumor in a subject in need thereof, the method comprising administering to the subject an effective amount of:
(i) at least one first chemically modified messenger RNA (mmRNA) encoding a polypeptide that induces immunogenic cell death, wherein said first mmRNA comprises one or more modified nucleobases;
and at least one of:
(ii) at least one second mmRNA encoding a polypeptide that potentiates an immune response, wherein said second mmRNA comprises one or more modified nucleobases; and/or
(iii) an immune checkpoint inhibitor, such that an immunogenic response to the tumor is generated in the subject.

E69. The method of embodiment 68, wherein the at least one first mmRNA encodes a polypeptide selected from the group consisting of MLKL, Diablo, RIPK3, and combinations thereof.

E70. The method of embodiment 69, wherein the first mmRNA encodes MLKL.

E71. The method of embodiment 69, wherein the first mmRNA encodes Diablo.

E72. The method of embodiment 69, wherein the first mmRNA encodes RIPK3.

E73. The method of embodiment 69, wherein the first mmRNA comprises two mmRNAs, one encoding MLKL and one encoding Diablo.

E74. The method of embodiment 69, wherein the first mmRNA comprises two mmRNAs, one encoding MLKL and one encoding RIPK3

E75. The method of embodiment 69, wherein the first mmRNA comprises two mmRNAs, one encoding RIPK3 and one encoding Diablo.

E76. The method of any one of embodiments 68-75, wherein the second mmRNA encodes STING.

E77. The method of any one of embodiments 68-76, wherein the immune checkpoint inhibitor is an anti-CTLA-4 antibody.

E78. The method of any one of embodiments 68-76, wherein the immune checkpoint inhibitor is an anti-PD-1 antibody.

E79. A chemically modified messenger RNA (mmRNA) encoding a polypeptide that enhances an immune response to an antigen of interest in a subject, wherein said mmRNA comprises one or more modified nucleobases, and wherein the immune response comprises a cellular or humoral immune response characterized by:
(i) stimulating Type I interferon pathway signaling;
(ii) stimulating NFkB pathway signaling;
(iii) stimulating an inflammatory response;
(iv) stimulating cytokine production; or
(v) stimulating dendritic cell development, activity or mobilization; and
(vi) a combination of any of (i)-(vi).

E80. The mmRNA of embodiment 79, wherein the antigen of interest is an endogenous antigen in the subject.

E81. The mmRNA of embodiment 79, wherein the antigen of interest is an exogenous antigen coadministered to the subject with the mmRNA.

E82. The mmRNA of embodiment 81 wherein the antigen of interest is encoded by an mmRNA.

E83. The mmRNA of any of embodiments 79-82, which encodes a constitutively active human STING polypeptide.

E84. The mmRNA of embodiment 83, wherein the constitutively active human STING polypeptide comprises one or more mutations selected from the group consisting of V147L, N154S, V155M, R284M, R284K, R284T, E315Q, R375A, and combinations thereof.

E85. The mmRNA of embodiment 84, wherein the constitutively active human STING polypeptide comprises a V155M mutation.

E86. The mmRNA of embodiment 84, wherein the constitutively active human STING polypeptide comprises mutations R284M/V147L/N154S/V155M.

E87. The mmRNA of embodiment 84, wherein the constitutively active human STING polypeptide comprises an amino acid sequence shown in any one of SEQ ID NOs: 1-10 or is encoded by a nucleotide sequence shown in any one of SEQ ID NOs: 199-208, 225, 1319 or 1320.

E88. The mmRNA of any one of embodiments 79-82, wherein the mmRNA encodes a constitutively active IRF3 polypeptide.

E89. The mmRNA of embodiment 88, wherein the constitutively active IRF3 polypeptide comprises an S396D mutation.

E90. The mmRNA of embodiment 89, wherein the constitutively active IRF3 polypeptide comprises an amino acid sequence shown in any one of SEQ ID NOs: 11-12.

E91. The mmRNA of any one of embodiments 79-82, wherein the mmRNA encodes a constitutively active human IRF7 polypeptide.

E92. The mmRNA of embodiment 91, wherein the constitutively active human IRF7 polypeptide comprises one or more mutations selected from the group consisting of S475D, S476D, S477D, S479D, L480D, S483D, S487D, deletion of amino acids 247-467, and combinations thereof.

E93. The mmRNA of embodiment 91, wherein the constitutively active human IRF7 polypeptide comprises an amino acid sequence shown in any one of SEQ ID NOs: 14-18.

E94. The mmRNA of any one of embodiments 79-82, wherein the polypeptide is selected from the group consisting of MyD88, TRAM, IRF1, IRF8, IRF9, TBK1, IKKi, STAT1, STAT2, STAT4, STAT6, c-FLIP, IKKβ, RIPK1, TAK-TAB1, DIABLO, Btk, self-activating caspase-1 and Flt3.

E95. The mmRNA of any one of embodiments 79-82, wherein the polypeptide stimulates Type I interferon pathway signaling.

E96. The mmRNA of any one of embodiments 79-82, wherein the polypeptide stimulates NFκB signaling.

E97. The mmRNA of any one of embodiments 79-82, wherein the polypeptide stimulates cytokine production.

E98. The mmRNA of any one of embodiments 79-82 wherein the immune response enhanced by the polypeptide is a cellular immune response.

E99. The mmRNA of any one of embodiments 79-82, wherein the immune response enhanced by the polypeptide is a humoral immune response.

E100. A composition comprising the mmRNA of any one of embodiments 79, 81-99 and a second mmRNA encoding at least one antigen of interest, wherein said second mmRNA comprises one or more modified nucleobases and wherein the polypeptide enhances an immune response to the at least one antigen of interest when the composition is administered to a subject.

E101. The composition of embodiment 100, which comprises a single mmRNA construct encoding both the at least one antigen of interest and the polypeptide that enhances an immune response to the at least one antigen of interest.

E102. The composition of embodiment 100, which comprises two mmRNA constructs, one encoding the at least one antigen of interest and the other encoding the polypeptide that enhances an immune response to the at least one antigen of interest.

E103. The composition of embodiment 102, wherein the two mmRNA constructs are coformulated in a lipid nanoparticle.

E104. The composition of any one of embodiments 100-103, wherein the at least one antigen of interest is at least one tumor antigen.

E105. The composition of embodiment 104, wherein the at least one tumor antigen is at least one mutant KRAS antigen.

E106. The composition of embodiment 105, wherein the at least one mutant KRAS antigen comprises at least one mutation selected from group consisting of G12D, G12V, G13D, G12C and combinations thereof.

E107. The composition of embodiment 105, wherein the at least one mutant KRAS antigen comprises an amino acid sequence shown in any one of SEQ ID NOs: 95-106 and 131-132 or is encoded by a nucleotide sequence shown in SEQ ID NO: 1321 or 1322.

E108. The composition of embodiment 105, which comprises an mmRNA encoding at least one mutant KRAS antigen and a constitutively active STING polypeptide, wherein the mmRNA encodes an amino acid sequence shown in any one of SEQ ID NOs: 107-130.

E109. The composition of any one of embodiment 100-103, wherein the at least one antigen of interest is at least one pathogen antigen.

E110. The composition of embodiment 109, wherein the at least one pathogen antigen is from a pathogen selected from the group consisting of viruses, bacteria, protozoa, fungi and parasites.

E111. The composition of embodiment 110, wherein the at least one pathogen antigen is at least one viral antigen.

E112. The composition of embodiment 111, wherein the at least one viral antigen is at least one human papillomavirus (HPV) antigen.

E113. The composition of embodiment 112, wherein the HPV antigen is an HPV16 E6 or HPV E7 antigen, or combination thereof.

E114. The composition of embodiment 113, wherein the HPV antigen comprises an amino acid sequence shown in any one of SEQ ID NOs: 36-94.

E115. The composition of embodiment 110, wherein the at least one pathogen antigen is at least one bacterial antigen.

E116. The mmRNA or composition of any one of embodiments 79-115 wherein the mmRNA(s) comprises a 5′ UTR, a codon optimized open reading frame encoding the polypeptide, a 3′ UTR and a 3′ tailing region of linked nucleosides.

E117. The mmRNA or composition of embodiment 116, wherein the mmRNA(s) further comprises one or more microRNA (miRNA) binding sites.

E118. The mmRNA or composition of any one of embodiments 79-117 wherein the mmRNA(s) is fully modified.

E119. The mmRNA or composition of any one of embodiments 79-118 wherein the mmRNA(s) comprises pseudouridine (w), pseudouridine (w) and 5-methyl-cytidine (m⁵C), 1-methyl-pseudouridine (m¹w), 1-methyl-pseudouridine (m¹w) and 5-methyl-cytidine (m⁵C), 2-thiouridine (s²U), 2-thiouridine and 5-methyl-cytidine (m⁵C), 5-methoxy-uridine (mo⁵U), 5-methoxy-uridine (mo⁵U) and 5-methyl-cytidine (m⁵C), 2′-O-methyl uridine, 2′-O-methyl uridine and 5-methyl-cytidine (m⁵C), N6-methyl-adenosine (m⁶A) or N6-methyl-adenosine (m⁶A) and 5-methyl-cytidine (m⁵C).

E120. The mmRNA or composition of any one of embodiments 79-119 wherein the mmRNA(s) comprises pseudouridine (w), N1-methylpseudouridine (m¹w), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine, or combinations thereof.

E121. The mmRNA or composition of any one of embodiments 79-120 wherein the mmRNA(s) comprises 1-methyl-pseudouridine (m¹w), 5-methoxy-uridine (mo⁵U), 5-methyl-cytidine (m⁵C), pseudouridine (w), α-thio-guanosine, or α-thio-adenosine, or combinations thereof.

E122. A lipid nanoparticle comprising the mmRNA or composition of any of embodiments 79-121.

E123. The lipid nanoparticle of embodiment 122, which is a liposome.

E124. The lipid nanoparticle of embodiment 122, which comprises a cationic and/or ionizable amino lipid.

E125. The lipid nanoparticle of embodiment 124, wherein the cationic and/or ionizable amino lipid is 2,2-dilinoleyl-4-methylaminoethyl-[1,3]-dioxolane (DLin-KC2-DMA) or dilinoleyl-methyl-4-dimethylaminobutyrate (DLin-MC3-DMA).

E126. The lipid nanoparticle of any one of embodiments 122-125, wherein the lipid nanoparticle further comprises a targeting moiety conjugated to the outer surface of the lipid nanoparticle.

E127. A pharmaceutical composition comprising the mmRNA or composition of any of embodiments 79-121 or the lipid nanoparticle of any one of embodiments 122-126, and a pharmaceutically acceptable carrier, diluent or excipient.

E128. A method for enhancing an immune response to an antigen of interest, the method comprising administering to a subject the mmRNA or composition of any one of embodiments 79-121, the lipid nanoparticle of any one of embodiments 122-126 or the pharmaceutical composition of embodiment 127 such that an immune response to the antigen of interest is enhanced in the subject.

E129. The method of embodiment 128, wherein enhancing an immune response comprises stimulating cytokine production.

E130. The method of embodiment 128, wherein enhancing an immune response comprises stimulating antigen-specific CD8+ T cell activity.

E131. The method of embodiment 128, wherein enhancing an immune response comprises stimulating antigen-specific antibody production.

E132. The method of embodiment 128, which comprises administering to the subject an mRNA composition that stimulates dendritic cell development or activity prior to administering to the subject an mRNA composition that stimulates Type I interferon pathway signaling.

E133. A method of stimulating an immunogenic response to a tumor in a subject in need thereof, the method comprising administering to the subject an effective amount of the mmRNA or the composition of any one of embodiments 79-121, or a lipid nanoparticle thereof, or a pharmaceutical composition thereof, such that an immunogenic response to the tumor is stimulated in the subject.

E134. The method of embodiment 133, wherein the tumor is a liver cancer, a colorectal cancer, a melanoma cancer, a pancreatic cancer, a non-small cell lung cancer (NSCLC), a cervical cancer or a head or neck cancer.

E135. The method of embodiment 133, wherein the subject is a human.

E136. A method of stimulating an immunogenic response to a pathogen in a subject in need thereof, the method comprising administering to the subject an effective amount of the mmRNA of any one of embodiments 79-99 and 116-121, or the composition of any one of embodiments 100-115, or a lipid nanoparticle thereof, or a pharmaceutical composition thereof, such that an immunogenic response to the pathogen is stimulated in the subject. JE137. The method of embodiment 136, wherein the pathogen is selected from the group consisting of viruses, bacteria, protozoa, fungi and parasites.

E138. The method of embodiment 137, wherein the pathogen is a virus.

E139. The method of embodiment 138, wherein the pathogen is human papillomavirus (HPV).

E140. The method of embodiment 137, wherein the pathogen is a bacteria.

E141. The method of embodiment 136, wherein the subject is a human.

E142. A method of preventing or treating an Human Papilloma Virus (HPV)-associated cancer in a subject in need thereof, the method comprising administering to the subject a composition comprising at least one mRNA construct encoding:(i) at least one HPV antigen of interest and (ii) a polypeptide that enhances an immune response against the at least one HPV antigen of interest, such that an immune response to the at least one HPV antigen of interest is enhanced.

E143. The method of embodiment 142, wherein the polypeptide that enhances an immune response against the at least one HPV antigen(s) of interest is a STING polypeptide.

E144. The method of embodiment 142, wherein the at least one HPV antigen is at least one E6 antigen, at least one E7 antigen or a combination of at least one E6 antigen and at least one E7 antigen.

E145. The method of embodiment 142, wherein the at least one HPV antigen and the polypeptide are encoded on separate mRNAs and are coformulated in a lipid nanoparticular prior to administration to the subject.

E146. The method embodiment 142, wherein the subject is at risk for exposure to HPV and the composition is administered prior to exposure to HPV.

E147. The method of embodiment 142, wherein the subject is infected with HPV or has an HPV-associated cancer.

E148. The method of embodiment 147, wherein the cancer is selected from the group consisting of cervical, penile, vaginal, vulval, anal and oropharyngeal cancers.

E149. The method of embodiment 148, wherein the subject is also treated with an immune checkpoint inhibitor.

E150. A composition comprising a first chemically modified messenger RNA (mmRNA) encoding a polypeptide that enhances an immune response to at least one oncogenic viral antigen of interest in a subject, and a second mmRNA encoding the at least one oncogenic viral antigen of interest, wherein each mmRNA comprises one or more modified nucleobases, and wherein the immune response comprises a cellular or humoral immune response characterized by:
(i) stimulating Type I interferon pathway signaling;
(ii) stimulating NFkB pathway signaling;
(iii) stimulating an inflammatory response;
(iv) stimulating cytokine production; or
(v) stimulating dendritic cell development, activity or mobilization; and
(vi) a combination of any of (i)-(vi).

E151. The composition of embodiment 150, which comprises a single mmRNA construct encoding both the at least one oncogenic viral antigen of interest and the polypeptide that enhances an immune response to the at least one oncogenic viral antigen of interest.

E152. The composition of embodiment 150 or 151, wherein the at least one oncogenic viral antigen of interest is derived from an oncogenic virus selected from the group consisting of: Human Papillomavirus (HPV), Hepatitis B virus (HBV), Hepatitis C virus (HCV), Epstein-barr virus (EBV), Human T-cell Lymphotropic virus type 1 (HTLV-1), Kaposi's sarcoma herpesvirus (KSHV) and Merkel cell polyomavirus (MCPyV).

E153. The composition of embodiment 150 or 151, wherein the at least one oncogenic viral antigen of interest is selected from the group of HPV antigens consisting of: E1, E2, E4, E5, E6, E7, L1, L2 and combinations thereof.

E154. The composition of embodiment 150 or 151, wherein the at least one oncogenic viral antigen of interest is selected from the group of HBV antigens consisting of: HBsAg, HBcAg, HBeAg, HBxAg, Pol, and combinations thereof.

E155. The composition of embodiment 150 or 151, wherein the at least one oncogenic viral antigen of interest is selected from the group of HCV antigens consisting of: Core (C, p22), E1 (gp35), E2 (gp70), NS1 (p7), NS2 (p23), NS3 (p70), NS4A (p8), NS4B (p27), NS5A (p56/58), NS5B (p68), and combinations thereof.

E156. The composition of embodiment 150 or 151, wherein the at least one oncogenic viral antigen of interest is an antigenic polypeptide from EBV-1 or EBV-2.

E157. The composition of embodiment 150 or 151, wherein the at least one oncogenic viral antigen of interest is selected from the group of HTLV-1 antigens consisting of: gag, pol, pro, env, tax, rex, p12, p21, p13, p30, HBZ, and combinations thereof.

E158. The composition of embodiment 150 or 151, wherein the at least one oncogenic viral antigen is an antigenic polypeptide from KSHV subtype A, KSHV subtype B, KSHV subtype C, KSHV subtype D, KSHV subtype E, or combinations thereof.

E159. The composition of embodiment 150 or 151, wherein the at least one oncogenic viral antigen of interest is selected from the group of MCPyV antigens consisting of: large T antigen (LT), small T antigen (sT), 57kT antigen (57kT), alternative T antigen (ALTO), major capsid protein viral protein 1 (VP1), the minor capsid viral proteins 2 or 3 (VP2 or VP3), and combinations thereof.

E160. The composition of any one of embodiments 150-159, wherein the at least one oncogenic viral antigen is a concatemeric oncogenic viral antigen comprised of 2-20 oncogenic viral antigens.

E161. The composition of embodiment 160, wherein the concatemeric oncogenic viral antigen comprises one or more of:
a) the 22-20 oncogenic viral antigens are interspersed by cleavage sensitive sites;
b) the mmRNA encoding each oncogenic viral antigen is linked directly to one another without a linker; and/or
c) the mmRNA encoding each oncogenic viral is linked to one or another with a single nucleotide linker.

E162. The composition of any one of embodiments 150-161, further comprising a ubiquitination signal.

E163. The composition of embodiment 162, wherein the ubiquitination signal is located at the C-terminus of the mmRNA.

E164. The composition of any one of embodiments 161-163, wherein at least one of the cleavage sites is an APC cleavage site.

E165. The composition of embodiment 164, wherein the cleavage site is a cleavage site for a serine protease, a threonine protease, a cysteine protease, an aspartate protease, a glutamic acid protease, or a metalloprotease.

E166. The composition of embodiment 165, wherein the cleavage site is for a cysteine protease.

E167. The composition of embodiment 166, wherein the cysteine protease is cathepsin B.

E168. The composition of embodiment 164, wherein the cleavage site comprises the amino acid sequence GFLG, Arg-↓-NHMec; Bz-Arg-↓-NhNap; Bz-Arg-↓NHMec; Bz-Phe-Cal-Arg-↓-NHMec; Pro-Gly-↓-Phe; Xaa-Xaa-Val-Val-Arg-Xaa-X or Arg-Arg, wherein Xaa is any amino acid residue.

E169. The composition of any one of embodiment 150-168, further comprising a recall antigen.

E170. The composition of embodiment 169, wherein the recall antigen is an mRNA having an open reading frame encoding the recall antigen.

E171. The composition of embodiment 169 or 170, wherein the recall antigen is included in the concatemeric antigen.

E172. The composition of any one of embodiment 150-171, further comprising an endosomal targeting sequence.

E173. The composition of embodiment 172, wherein the endosomal targeting sequence comprises at least a portion of the transmembrane domain of lysosome associated membrane protein (LAMP-1).

E174. The composition of embodiment 172, wherein the endosomal targeting sequence comprises at least a portion of the transmembrane domain of invariant chain (Ii).

E175. A composition comprising a first chemically modified messenger RNA (mmRNA) encoding a polypeptide that enhances an immune response to at least one antigen derived from HPV, and a second mmRNA encoding the at least one antigen derived from HPV, wherein each mmRNA comprises one or more modified nucleobases.

E176. The composition of embodiment 175, wherein the second mmRNA encodes HPV antigen E6 and/or HPV antigen E7.

E177. The composition of embodiment 175 or 176, wherein the first mmRNA encodes a constitutively active human STING polypeptide.

E178. The composition of any one of embodiment 150-177, wherein each mmRNA is formulated in the same or different lipid nanoparticle.

E179. The composition of embodiment 178, wherein each mmRNA encoding an oncogenic viral antigen is formulated in the same or different lipid nanoparticle.

E180. The composition of embodiment 179, wherein each mmRNA encoding a polypeptide that enhances an immune response to the oncogenic viral antigen is formulated in the same or different lipid nanoparticle.

E181. The composition of any one of embodiments 178-180, wherein each mmRNA encoding an oncogenic viral antigen is formulated in the same lipid nanoparticle, and each mmRNA encoding a polypeptide that enhances an immune response to the oncogenic viral antigen is formulated in a different lipid nanoparticle.

E182. The composition of any one of embodiments 178-180, wherein each mmRNA encoding an oncogenic viral antigen is formulated in the same lipid nanoparticle, and each mmRNA encoding a polypeptide that enhances an immune response to the oncogenic viral antigen is formulated in the same lipid nanoparticle as each mmRNA encoding an oncogenic viral antigen.

E183. The composition of any one of embodiments 178-180, wherein each mmRNA encoding an oncogenic viral antigen is formulated in a different lipid nanoparticle, and each mmRNA encoding a polypeptide that enhances an immune response to the oncogenic viral antigen is formulated in the same lipid nanoparticle as each mmRNA encoding each oncogenic viral antigen.

E184. A lipid nanoparticle carrier comprising a pharmaceutical composition, wherein the pharmaceutical composition comprises:
an mmRNA having an open reading frame encoding a concatemer of oncogenic viral antigens;
an mmRNA having an open reading frame encoding a polypeptide that enhances an immune response to the concatemer of oncogenic viral antigens; and
a pharmaceutically acceptable carrier or excipient.

E185. A lipid nanoparticle carrier comprising a pharmaceutical composition, wherein the pharmaceutical composition comprises:
at least one mmRNA having an open reading frame encoding an oncogenic viral antigen;
an mmRNA having an open reading frame encoding a polypeptide that enhances an immune response to the oncogenic viral antigen; and
a pharmaceutically acceptable carrier or excipient.

E186. A lipid nanoparticle carrier comprising a pharmaceutical composition, wherein the pharmaceutical composition comprises:
an mmRNA having an open reading frame encoding a concatemer of HPV antigens;
an mmRNA having an open reading frame encoding a constitutively active human STING polypeptide; and
a pharmaceutically acceptable carrier or excipient.

E187. The lipid nanoparticle carrier of embodiment 186, wherein the concatemer of HPV antigens comprises HPV antigens E6 and E7.

E188. A lipid nanoparticle carrier comprising a pharmaceutical composition, wherein the pharmaceutical composition comprises:
an mmRNA having an open reading frame encoding HPV viral antigen E6;
an mmRNA having an open reading frame encoding HPV viral antigen E7;
an mmRNA having an open reading frame encoding a constitutively active human STING polypeptide; and
a pharmaceutically acceptable carrier or excipient E189. A vaccine comprising:
a first nanoparticle comprising a pharmaceutical composition, wherein the pharmaceutical composition comprises an mmRNA having an open reading frame encoding a first oncogenic viral antigen of interest, an mmRNA having an open reading frame encoding a polypeptide that enhances an immune response to the first oncogenic viral antigen of interest, and a pharmaceutically acceptable carrier or excipient;
a second nanoparticle comprising a pharmaceutical composition, wherein the pharmaceutical composition comprises an mmRNA having an open reading frame encoding a second oncogenic viral antigen of interest, an mmRNA having an open reading frame encoding a polypeptide that enhances an immune response to the second oncogenic viral antigen of interest, and a pharmaceutically acceptable carrier or excipient;
a third nanoparticle comprising a pharmaceutical composition, wherein the pharmaceutical composition comprises an mmRNA having an open reading frame encoding a third oncogenic viral antigen of interest, an mmRNA having an open reading frame encoding a polypeptide that enhances an immune response to the third oncogenic viral antigen of interest, and a pharmaceutically acceptable carrier or excipient;
a fourth nanoparticle comprising a pharmaceutical composition, wherein the pharmaceutical composition comprises an mmRNA having an open reading frame encoding a fourth oncogenic viral antigen of interest, an mmRNA having an open reading frame encoding a polypeptide that enhances an immune response to the fourth oncogenic viral antigen of interest, and a pharmaceutically acceptable carrier or excipient; or
a combination thereof.

E190. A vaccine comprising:
a nanoparticle comprising a pharmaceutical composition, wherein the pharmaceutical composition comprises an mmRNA having an open reading frame encoding a concatemeric oncogenic viral antigen of interest, an mmRNA having an open reading frame encoding a polypeptide that enhances an immune response to the concatemeric oncogenic viral antigen of interest, and a pharmaceutically acceptable carrier or excipient.

E191. A vaccine comprising:
a first nanoparticle comprising a pharmaceutical composition, wherein the pharmaceutical composition comprises an mmRNA having an open reading frame encoding HPV antigen E6, an mmRNA having an open reading frame encoding a constitutively active human STING polypeptide, and a pharmaceutically acceptable carrier or excipient; and
a second nanoparticle comprising a pharmaceutical composition, wherein the pharmaceutical composition comprises an mmRNA having an open reading frame encoding HPV antigen E7, an mmRNA having an open reading frame encoding a constitutively active human STING polypeptide, and a pharmaceutically acceptable carrier or excipient.

E192. A method of preventing tumor growth in a subject infected with an oncogenic virus, comprising administering to the subject the composition, lipid nanoparticle carrier, or vaccine of any one of embodiments 150-191, such that tumor growth is prevented in the subject.

E193. The method of embodiment 192, wherein the subject has no detectable tumor prior to administration.

E194. A method of inhibiting tumor growth in a subject infected with an oncogenic virus, comprising administering to the subject the composition, lipid nanoparticle carrier, or vaccine of any one of embodiment 150-191, such that tumor growth is inhibited in the subject.

E195. The method of embodiment 194, wherein tumor formation prior to administration is a result of infection with the oncogenic virus.

E196. A method of treating cancer in a cancer subject infected with an oncogenic virus, comprising administering to the subject the composition, lipid nanoparticle carrier, or vaccine of any one of embodiments 150-191, such that cancer is treated in the subject.

E197. The method of embodiment 196, wherein the cancer is a result of infection with the oncogenic virus.

E198. A personalized cancer vaccine comprising a first chemically modified messenger RNA (mmRNA) encoding a polypeptide that enhances an immune response to at least one cancer antigen of interest in a subject, and a second mmRNA encoding the at least one cancer antigen of interest, wherein each mmRNA comprises one or more modified nucleobases, and wherein the immune response comprises a cellular or humoral immune response characterized by:
(i) stimulating Type I interferon pathway signaling;
(ii) stimulating NFκB pathway signaling;
(iii) stimulating an inflammatory response;
(iv) stimulating cytokine production; or
(v) stimulating dendritic cell development, activity or mobilization; and
(vi) a combination of any of (i)-(vi).

E199. The personalized cancer vaccine of embodiment 198, which comprises a single mmRNA construct encoding both the at least one cancer antigen of interest and the polypeptide that enhances an immune response to the at least one cancer antigen of interest.

E200. The personalized cancer vaccine of embodiment 198 or 199, wherein the at least one cancer antigen of interest is a concatemeric cancer antigen comprised of 2-100 peptide epitopes.

E201. The personalized cancer vaccine of embodiment 200, wherein the concatemeric cancer antigen comprises one or more of:
a) the 2-100 peptide epitopes are interspersed by cleavage sensitive sites;
b) the mRNA encoding each peptide epitope is linked directly to one another without a linker;
c) the mRNA encoding each peptide epitope is linked to one or another with a single nucleotide linker;
d) each peptide epitope comprises 25-35 amino acids and includes a centrally located SNP mutation;
e) at least 30% of the peptide epitopes have a highest affinity for class I MHC molecules from a subject;
f) at least 30% of the peptide epitopes have a highest affinity for class II MHC molecules from a subject;
g) at least 50% of the peptide epitopes have a predicated binding affinity of IC >500 nM for HLA-A, HLA-B and/or DRB1;
h) the mRNA encodes 20 peptide epitopes;
i) 50% of the peptide epitopes have a binding affinity for class I MHC and 50% of the peptide epitopes have a binding affinity for class II MHC; and/or
j) the mRNA encoding the peptide epitopes is arranged such that the peptide epitopes are ordered to minimize pseudo-epitopes.

E202. The personalized cancer vaccine of embodiment 201, wherein each peptide epitope comprises 31 amino acids and includes a centrally located SNP mutation with 15 flanking amino acids on each side of the SNP mutation.

E203. The personalized cancer vaccine of any one of embodiments 200-202, wherein the peptide epitopes are T cell epitopes and/or B cell epitopes.

E204. The personalized cancer vaccine of any one of embodiments 200-203, wherein the peptide epitopes comprise a combination of T cell epitopes and B cell epitopes.

E205. The personalized cancer vaccine of any one of embodiments 200-204, wherein at least 1 of the peptide epitopes is a T cell epitope.

E206. The personalized cancer vaccine of any one of embodiments 200-205, wherein at least 1 of the peptide epitopes is a B cell epitope.

E207. The personalized cancer vaccine of any one of embodiments 200-206 wherein the T cell epitope comprises between 8-11 amino acids.

E208. The personalized cancer vaccine of any one of embodiments 200-207, wherein the B cell epitope comprises between 13-17 amino acids.

E209. The personalized cancer vaccine of any one of embodiments 198-208, further comprising a ubiquitination signal.

E210. The personalized cancer vaccine of embodiment 209, wherein the ubiquitination signal is located at the C-terminus of the mmRNA.

E211. The personalized cancer vaccine of any one of embodiments 201-210, wherein at least one of the cleavage sensitive sites is an APC cleavage site.

E212. The personalized cancer vaccine of embodiment 211, wherein the cleavage site is a cleavage site for a serine protease, a threonine protease, a cysteine protease, an aspartate protease, a glutamic acid protease, or a metalloprotease.

E213. The personalized cancer vaccine of embodiment 212, wherein the cleavage site is for a cysteine protease.

E214. The personalized cancer vaccine of embodiment 213, wherein the cysteine protease is cathepsin B.

E215. The personalized cancer vaccine of embodiment 214, wherein the cleavage site comprises the amino acid sequence GFLG, Arg-θ-NHMec; Bz-Arg-↓-NhNap; Bz-Arg-↓NHMec; Bz-Phe-Cal-Arg-↓-NHMec; Pro-Gly-↓-Phe; Xaa-Xaa-Val-Val-Arg-Xaa-X or Arg-Arg, wherein Xaa is any amino acid residue.

E216. The personalized cancer vaccine of any one of embodiments 200-215, wherein each peptide epitope comprises an antigenic region and a MHC stabilizing region.

E217. The personalized cancer vaccine of embodiment 216, wherein the MHC stabilizing region is 5-10 amino acids in length.

E218. The personalized cancer vaccine of embodiment 216 or 217, wherein the antigenic region is 5-100 amino acids in length.

E219. The personalized cancer vaccine of any one of embodiments 200-218, wherein the peptide epitopes have been optimized for binding strength to a MHC of the subject.

E220. The personalized cancer vaccine of embodiment 219, wherein a TCR face for each epitope has a low similarity to endogenous proteins.

E221. The personalized cancer vaccine of any one of embodiments 198-220, further comprising a recall antigen.

E222. The personalized cancer vaccine of embodiment 221, wherein the recall antigen is an infectious disease antigen.

E223. The personalized cancer vaccine of embodiment 221 or 222, wherein the recall antigen is an mRNA having an open reading frame encoding the recall antigen.

E224. The personalized cancer vaccine of any one of embodiments 221-223, wherein the recall antigen is a peptide epitope in the concatemeric antigen.

E225. The personalized cancer vaccine of any one of embodiments 221 and 223-224, wherein the recall antigen is an influenza antigen.

E226. The personalized cancer vaccine of any one of embodiments 198-225, further comprising an endosomal targeting sequence.

E227. The personalized cancer vaccine of embodiment 226, wherein the endosomal targeting sequence comprises at least a portion of the transmembrane domain of lysosome associated membrane protein (LAMP-1).

E228. The personalized cancer vaccine of embodiment 226, wherein the endosomal targeting sequence comprises at least a portion of the transmembrane domain of invariant chain (Ii).

E229. The personalized cancer vaccine of embodiment 200, wherein the peptide epitopes comprise at least one MHC class I epitope and at least one MHC class II epitope.

E230. The personalized cancer vaccine of embodiment 229, wherein at least 30% of the epitopes are MHC class I epitopes.

E231. The personalized cancer vaccine of embodiment 229, wherein at least 30% of the epitopes are MHC class II epitopes.

E232. The personalized cancer vaccine of any one of embodiment 198-231, further comprising an ORF encoding one or more traditional cancer antigens.

E233. The personalized cancer vaccine of any one of embodiments 198-232, further comprising an mRNA having an open reading frame encoding one or more traditional cancer antigens.

E234. The personalized cancer vaccine of any one of embodiments 198-233, wherein the polypeptide that enhances an immune response to at least one cancer antigen of interest in a subject is a constitutively active human STING polypeptide.

E235. The personalized cancer vaccine of embodiment 234, wherein the constitutively active human STING polypeptide comprises one or more mutations selected from the group consisting of V147L, N154S, V155M, R284M, R284K, R284T, E315Q, R375A, and combinations thereof.

E236. The personalized cancer vaccine of embodiment 235, wherein the constitutively active human STING polypeptide comprises a V155M mutation.

E237. The personalized cancer vaccine of embodiment 235, wherein the constitutively active human STING polypeptide comprises mutations R284M/V147L/N154S/V155M.

E238. A composition comprising the personalized cancer vaccine of any one of embodiments 198-238.

E239. The composition of embodiment 238, wherein each mmRNA is formulated in the same or different lipid nanoparticle.

E240. The composition of embodiment 239, wherein each mmRNA encoding a cancer antigen of interest is formulated in the same or different lipid nanoparticle.

E241. The composition of embodiment 240, wherein each mmRNA encoding a polypeptide that enhances an immune response to the cancer antigen of interest is formulated in the same or different lipid nanoparticle.

E242. The composition of any one of embodiments 239-241, wherein each mmRNA encoding a cancer antigen of interest is formulated in the same lipid nanoparticle, and each mmRNA encoding a polypeptide that enhances an immune response to the cancer antigen of interest is formulated in a different lipid nanoparticle.

E243. The composition of any one of embodiments 239-241, wherein each mmRNA encoding a cancer antigen of interest is formulated in the same lipid nanoparticle, and each mmRNA encoding a polypeptide that enhances an immune response to the cancer antigen of interest is formulated in the same lipid nanoparticle as each mmRNA encoding a cancer antigen of interest.

E244. The composition of any one of embodiments 239-241, wherein each mmRNA encoding a cancer antigen of interest is formulated in a different lipid nanoparticle, and each mmRNA encoding a polypeptide that enhances an immune response to the cancer antigen of interest is formulated in the same lipid nanoparticle as each mmRNA encoding each cancer antigen of interest.

E245. A lipid nanoparticle carrier comprising a pharmaceutical composition, wherein the pharmaceutical composition comprises:
an mmRNA having an open reading frame encoding a concatemeric cancer antigen of interest;
an mmRNA having an open reading frame encoding a polypeptide that enhances an immune response to the concatemeric cancer antigen of interest;
and a pharmaceutically acceptable carrier or excipient.

E246. A lipid nanoparticle carrier comprising a pharmaceutical composition, wherein the pharmaceutical composition comprises:
at least one mmRNA having an open reading frame encoding a cancer antigen of interest;
an mmRNA having an open reading frame encoding a polypeptide that enhances an immune response to the cancer antigen of interest; and
a pharmaceutically acceptable carrier or excipient.

E247. A personalized cancer vaccine comprising:
a lipid nanoparticle comprising a pharmaceutical composition, wherein the pharmaceutical composition comprises at least one mmRNA having an open reading frame encoding a cancer antigen of interest in a subject, an mmRNA having an open reading frame encoding a polypeptide that enhances an immune response to the cancer antigen of interest, and a pharmaceutically acceptable carrier or excipient.

E248. A personalized cancer vaccine comprising:
a lipid nanoparticle comprising a pharmaceutical composition, wherein the pharmaceutical composition comprises at least one mmRNA having an open reading frame encoding a concatemeric cancer antigen of interest, an mmRNA having an open reading frame encoding a polypeptide that enhances an immune response to the cancer antigen of interest, and a pharmaceutically acceptable carrier or excipient.

E249. A method for vaccinating a subject, comprising:
administering to a subject having cancer a personalized cancer vaccine or composition of any one of embodiments 198-248 in order to vaccinate the subject.

E250. A method for treating a subject with a personalized cancer vaccine, comprising isolating a sample from the subject, identifying a set of neoepitopes by analyzing a patient transcriptome and/or a patient exome from the sample to produce a patient specific mutanome, selecting a set of neoepitopes for the vaccine from the mutanome based on MHC binding strength, MHC binding diversity, predicted degree of immunogenicity, low self reactivity, and/or T cell reactivity, preparing a mRNA to encode the set of neoepitopes and a polypeptide that enhances an immune response to the neoepitopes, and administering the personalized cancer vaccine to the subject within two months of isolating the sample from the subject.

E251. The method of embodiment 250, wherein the personalized cancer vaccine is administered to the subject within one month of isolating the sample from the subject.

E252. The method of embodiment 250 or 251, wherein the personalized cancer vaccine further encodes one or more traditional cancer antigens.

E253. The method of embodiment 252, wherein the one or more traditional cancer antigens are encoded by the same mRNA that encode the set of neoepitopes.

E254. The method of embodiment 252, wherein the one or more traditional cancer antigens are encoded by a different mRNA than the mRNA which encodes the set of neoeptiopes.

E255. The method of any one of embodiments 250-254, wherein the personalized cancer vaccine is administered in combination with a cancer therapeutic agent.

E256. The method of embodiment 255, wherein the cancer therapeutic agent is a traditional cancer vaccine.

E257. A bacterial vaccine comprising a first chemically modified messenger RNA (mmRNA) encoding a polypeptide that enhances an immune response to at least one bacterial antigen of interest, and a second mmRNA encoding the at least one bacterial antigen of interest, wherein each mmRNA comprises one or more modified nucleobases, and wherein the immune response comprises a cellular or humoral immune response characterized by:
(i) stimulating Type I interferon pathway signaling;
(ii) stimulating NFκB pathway signaling;
(iii) stimulating an inflammatory response;
(iv) stimulating cytokine production; or
(v) stimulating dendritic cell development, activity or mobilization; and
(vi) a combination of any of (i)-(vi).

E258. The bacterial vaccine of embodiment 257, which comprises a single mmRNA construct encoding both the at least one bacterial antigen of interest and the polypeptide that enhances an immune response to the at least one bacterial antigen of interest.

E259. The bacterial vaccine of embodiment 257 or 258, wherein the at least one bacterial antigen of interest is a concatemeric bacterial antigen comprised of 2-10 bacterial antigens.

E260. The bacterial vaccine of embodiment 259, wherein the concatemeric bacterial antigen comprises one or more of:
a) the 2-10 bacterial antigens are interspersed by cleavage sensitive sites;
b) the mmRNA encoding each bacterial antigen is linked directly to one another without a linker; and/or
c) the mmRNA encoding each bacterial antigen is linked to one or another with a single nucleotide linker.

E261. The bacterial vaccine of any one of embodiments 257-260, further comprising a ubiquitination signal.

E262. The bacterial vaccine of embodiment 261, wherein the ubiquitination signal is located at the C-terminus of the mmRNA.

E263. The bacterial vaccine of any one of embodiments 260-262, wherein at least one of the cleavage sites is an APC cleavage site.

E264. The bacterial vaccine of embodiment 263, wherein the cleavage site is a cleavage site for a serine protease, a threonine protease, a cysteine protease, an aspartate protease, a glutamic acid protease, or a metalloprotease.

E265. The bacterial vaccine of embodiment 264, wherein the cleavage site is for a cysteine protease.

E266. The bacterial vaccine of embodiment 265, wherein the cysteine protease is cathepsin B.

E267. The bacterial vaccine of embodiment 263, wherein the cleavage site comprises the amino acid sequence GFLG, Arg-↓-NHMec; Bz-Arg-↓-NhNap; Bz-Arg-INHMec; Bz-Phe-Cal-Arg-↓-NHMec; Pro-Gly-↓-Phe; Xaa-Xaa-Val-Val-Arg-Xaa-X or Arg-Arg, wherein Xaa is any amino acid residue.

E268. The bacterial vaccine of any one of embodiments 257-267, further comprising a recall antigen.

E269. The bacterial vaccine of embodiment 268, wherein the recall antigen is an infectious disease antigen.

E270. The bacterial vaccine of embodiment 268 or 269, wherein the recall antigen is an mRNA having an open reading frame encoding the recall antigen.

E271. The bacterial vaccine of any one of embodiments 268-270, wherein the recall antigen is included in the concatemeric antigen.

E272. The bacterial vaccine of anyone of embodiments 268-271, wherein the recall antigen is an influenza antigen.

E273. The bacterial vaccine of any one of embodiments 257-272, further comprising an endosomal targeting sequence.

E274. The bacterial vaccine of embodiment 273, wherein the endosomal targeting sequence comprises at least a portion of the transmembrane domain of lysosome associated membrane protein (LAMP-1).

E275. The bacterial vaccine of embodiment 273, wherein the endosomal targeting sequence comprises at least a portion of the transmembrane domain of invariant chain (Ii).

E276. The bacterial vaccine of any one of embodiments 257-275, wherein the vaccine induces a humoral immune response.

E277. The bacterial vaccine of any one of embodiments 257-275, wherein the vaccine induces an adaptive immune response.

E278. The bacterial vaccine of embodiment 277, wherein the adaptive immune response comprises induction of antigen-specific antibody production or antigen-specific induction/activation of T helper lymphocytes or cytotoxic lymphocytes.

E279. The bacterial vaccine of any one of embodiments 257-278, wherein the bacterial antigen of interested is derived from Staphylococcus aureus.

E280. A composition comprising the bacterial vaccine of any one of embodiments 257-279.

E281. The composition of embodiment 280, wherein each mmRNA is formulated in the same or different lipid nanoparticle.

E282. The composition of embodiment 281, wherein each mmRNA encoding a bacterial antigen of interest is formulated in the same or different lipid nanoparticle.

E283. The composition of embodiment 282, wherein each mmRNA encoding a polypeptide that enhances an immune response to the bacterial antigen of interest is formulated in the same or different lipid nanoparticle.

E284. The composition of any one of embodiments 281-283, wherein each mmRNA encoding a bacterial antigen of interest is formulated in the same lipid nanoparticle, and each mmRNA encoding a polypeptide that enhances an immune response to the bacterial antigen is formulated in a different lipid nanoparticle.

E285. The composition of any one of embodiments 281-283, wherein each mmRNA encoding a bacterial antigen of interest is formulated in the same lipid nanoparticle, and each mmRNA encoding a polypeptide that enhances an immune response to the bacterial antigen is formulated in the same lipid nanoparticle as each mmRNA encoding a bacterial antigen.

E286. The composition of any one of embodiments 281-283, wherein each mmRNA encoding a bacterial antigen is formulated in a different lipid nanoparticle, and each mmRNA encoding a polypeptide that enhances an immune response to the bacterial antigen is formulated in the same lipid nanoparticle as each mmRNA encoding each bacterial antigen.

E287. A lipid nanoparticle carrier comprising a pharmaceutical composition, wherein the pharmaceutical composition comprises:
an mmRNA having an open reading frame encoding a concatemer of bacterial antigens;
an mmRNA having an open reading frame encoding a polypeptide that enhances an immune response to the concatemer of bacterial antigens; and
a pharmaceutically acceptable carrier or excipient.

E288. A lipid nanoparticle carrier comprising a pharmaceutical composition, wherein the pharmaceutical composition comprises:
at least one mmRNA having an open reading frame encoding bacterial antigen;
an mmRNA having an open reading frame encoding a polypeptide that enhances an immune response to the bacterial antigen; and
a pharmaceutically acceptable carrier or excipient.

E289. A bacterial vaccine comprising:
a nanoparticle comprising a pharmaceutical composition, wherein the pharmaceutical composition comprises an mmRNA having an open reading frame encoding a bacterial antigen of interest, an mmRNA having an open reading frame encoding a polypeptide that enhances an immune response to the bacterial antigen of interest, and a pharmaceutically acceptable carrier or excipient.

E290. A bacterial vaccine comprising:
a nanoparticle comprising a pharmaceutical composition, wherein the pharmaceutical composition comprises an mmRNA having an open reading frame encoding a concatemeric bacterial antigen of interest, an mmRNA having an open reading frame encoding a polypeptide that enhances an immune response to the concatemeric bacterial antigen of interest, and a pharmaceutically acceptable carrier or excipient.

E291. A method for vaccinating a subject against infection by a bacterium of interest, comprising:
administering to the subject a bacterial vaccine, composition, or lipid nanoparticle carrier of any one of embodiments 257-290 in order to vaccinate the subject. E292. The method of embodiment 291, wherein the bacterium of interest is Staphylococcus aureus.

E293. The method of embodiment 291, wherein the bacterium of interest is Methicillin Resistant Staphylococcus aureus (MRSA).

E294. A method for treating a subject with a bacterial infection, comprising:
administering to the subject a bacterial vaccine, composition, or lipid nanoparticle carrier of any one of embodiments 257-290 in order to treat the subject.

E295. The method of embodiment 294, wherein the bacterial infection is caused by Staphylococcus aureus.

E296. The method of embodiment 294, wherein the bacterial infection is caused by Methicillin Resistant Staphylococcus aureus (MRSA).

### Definitions

Administering: As used herein, “administering” refers to a method of delivering a composition to a subject or patient. A method of administration may be selected to target delivery (e.g., to specifically deliver) to a specific region or system of a body. For example, an administration may be parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g., by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter.

Approximately, about: As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Cancer: As used herein, “cancer” is a condition involving abnormal and/or unregulated cell growth. The term cancer encompasses benign and malignant cancers. Exemplary non-limiting cancers include adrenal cortical cancer, advanced cancer, anal cancer, aplastic anemia, bileduct cancer, bladder cancer, bone cancer, bone metastasis, brain tumors, brain cancer, breast cancer, childhood cancer, cancer of unknown primary origin, Castleman disease, cervical cancer, colorectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, acute lymphocytic leukemia, acute myeloid leukemia, chronic lymphocytic leukemia, chronic myeloid leukemia, chronic myelomonocytic leukemia, myelodysplastic syndrome (including refractory anemias and refractory cytopenias), myeloproliferative neoplasms or diseases (including polycythemia vera, essential thrombocytosis and primary myelofibrosis), liver cancer (e.g., hepatocellular carcinoma), non-small cell lung cancer, small cell lung cancer, lung carcinoid tumor, lymphoma of the skin, malignant mesothelioma, multiple myeloma, myelodysplasia syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment. In particular embodiments, the cancer is liver cancer (e.g., hepatocellular carcinoma) or colorectal cancer. In other embodiments, the cancer is a blood-based cancer or a hematopoetic cancer.

Cleavable Linker: As used herein, the term “cleavable linker” refers to a linker, typically a peptide linker (e.g., about 5-30 amino acids in length, typically about 10-20 amino acids in length) that can be incorporated into multicistronic mRNA constructs such that equimolar levels of multiple genes can be produced from the same mRNA. Non-limiting examples of cleavable linkers include the 2A family of peptides, including F2A, P2A, T2A and E2A, first discovered in picornaviruses, that when incorporated into an mRNA construct (e.g., between two polypeptide domains) function by making the ribosome skip the synthesis of a peptide bond at C-terminus of the 2A element, thereby leading to separation between the end of the 2A sequence and the next peptide downstream.

Conjugated: As used herein, the term “conjugated,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, two or more moieties may be conjugated by direct covalent chemical bonding. In other embodiments, two or more moieties may be conjugated by ionic bonding or hydrogen bonding.

Contacting: As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., an isolated mRNA, nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

Encapsulate: As used herein, the term “encapsulate” means to enclose, surround, or encase. In some embodiments, a compound, polynucleotide (e.g., an mRNA), or other composition may be fully encapsulated, partially encapsulated, or substantially encapsulated. For example, in some embodiments, an mRNA of the disclosure may be encapsulated in a lipid nanoparticle, e.g., a liposome.

Effective amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent. In some embodiments, a therapeutically effective amount is an amount of an agent to be delivered (e.g., nucleic acid, drug, therapeutic agent, diagnostic agentor prophylactic agent) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux et al., Nucleic Acids Research, 12 (1): 387,1984, BLASTP, BLASTN, and FASTA, Altschul, S. F. et al., J. Molec. Biol., 215, 403, 1990.

Fragment: A “fragment,” as used herein, refers to a portion. For example, fragments of proteins may include polypeptides obtained by digesting full-length protein isolated from cultured cells or obtained through recombinant DNA techniques.

GC-rich: As used herein, the term “GC-rich” refers to the nucleobase composition of a polynucleotide (e.g., mRNA), or any portion thereof (e.g., an RNA element), comprising guanine (G) and/or cytosine (C) nucleobases, or derivatives or analogs thereof, wherein the GC-content is greater than about 50%. The term “GC-rich” refers to all, or to a portion, of a polynucleotide, including, but not limited to, a gene, a non-coding region, a 5′ UTR, a 3′ UTR, an open reading frame, an RNA element, a sequence motif, or any discrete sequence, fragment, or segment thereof which comprises about 50% GC-content. In some embodiments of the disclosure, GC-rich polynucleotides, or any portions thereof, are exclusively comprised of guanine (G) and/or cytosine (C) nucleobases.

GC-content: As used herein, the term “GC-content” refers to the percentage of nucleobases in a polynucleotide (e.g., mRNA), or a portion thereof (e.g., an RNA element), that are either guanine (G) and cytosine (C) nucleobases, or derivatives or analogs thereof, (from a total number of possible nucleobases, including adenine (A) and thymine (T) or uracil (U), and derivatives or analogs thereof, in DNA and in RNA). The term “GC-content” refers to all, or to a portion, of a polynucleotide, including, but not limited to, a gene, a non-coding region, a 5′ or 3′ UTR, an open reading frame, an RNA element, a sequence motif, or any discrete sequence, fragment, or segment thereof.

Genetic Adjuvant: A “genetic adjuvant”, as used herein, refers to an mRNA construct (e.g., an mmRNA construct) that enhances the immune response to a vaccine, for example by stimulating cytokine production and/or by stimulating the production of antigen-specific effector cells (e.g., CD8 T cells). A genetic adjuvant mRNA construct can, for example, encode a polypeptide that stimulates Type I interferon (e.g., activates Type I interferon pathway signaling) or that promotes dendritic cell development or activity.

Heterologous: As used herein, “heterologous” indicates that a sequence (e.g., an amino acid sequence or the polynucleotide that encodes an amino acid sequence) is not normally present in a given polypeptide or polynucleotide. For example, an amino acid sequence that corresponds to a domain or motif of one protein may be heterologous to a second protein.

Hydrophobic amino acid: As used herein, a “hydrophobic amino acid” is an amino acid having an uncharged, nonpolar side chain. Examples of naturally occurring hydrophobic amino acids are alanine (Ala), valine (Val), leucine (Leu), isoleucine (Ile), proline (Pro), phenylalanine (Phe), methionine (Met), and tryptophan (Trp).

Immune Potentiator: An “immune potentiator”, as used herein, refers to an mRNA construct (e.g., an mmRNA construct) that enhances an immune response, e.g., to an antigen of interest (either an endogenous antigen in a subject to which the immune potentiator is administered or to an exogenous antigen that is coadministered with the immune potentiator), for example by stimulating T cell, B cell or dendritic cell responses, including but not limited to cytokine production, stimulating antibody production or stimulating the production of antigen-specific immune cells (e.g., CD8⁺ T cells or CD4⁺ T cells).

Initiation Codon: As used herein, the term “initiation codon”, used interchangeably with the term “start codon”, refers to the first codon of an open reading frame that is translated by the ribosome and is comprised of a triplet of linked adenine-uracil-guanine nucleobases. The initiation codon is depicted by the first letter codes of adenine (A), uracil (U), and guanine (G) and is often written simply as “AUG”. Although natural mRNAs may use codons other than AUG as the initiation codon, which are referred to herein as “alternative initiation codons”, the initiation codons of polynucleotides described herein use the AUG codon. During the process of translation initiation, the sequence comprising the initiation codon is recognized via complementary base-pairing to the anticodon of an initiator tRNA (Met-tRNAᵢ^{Met}) bound by the ribosome. Open reading frames may contain more than one AUG initiation codon, which are referred to herein as “alternate initiation codons”.

The initiation codon plays a critical role in translation initiation. The initiation codon is the first codon of an open reading frame that is translated by the ribosome. Typically, the initiation codon comprises the nucleotide triplet AUG, however, in some instances translation initiation can occur at other codons comprised of distinct nucleotides. The initiation of translation in eukaryotes is a multistep biochemical process that involves numerous protein-protein, protein-RNA, and RNA-RNA interactions between messenger RNA molecules (mRNAs), the 40S ribosomal subunit, other components of the translation machinery (e.g., eukaryotic initiation factors; eIFs). The current model of mRNA translation initiation postulates that the pre-initiation complex (alternatively “43S pre-initiation complex”; abbreviated as “PIC”) translocates from the site of recruitment on the mRNA (typically the 5′ cap) to the initiation codon by scanning nucleotides in a 5′ to 3′ direction until the first AUG codon that resides within a specific translation-promotive nucleotide context (the Kozak sequence) is encountered (Kozak (1989) J Cell Biol 108:229-241). Scanning by the PIC ends upon complementary base-pairing between nucleotides comprising the anticodon of the initiator Met-tRNA Met transfer RNA and nucleotides comprising the initiation codon of the mRNA. Productive base-pairing between the AUG codon and the Met-tRNAᵢ^{Met} anticodon elicits a series of structural and biochemical events that culminate in the joining of the large 60S ribosomal subunit to the PIC to form an active ribosome that is competent for translation elongation.

Insertion: As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule. For example, an amino acid sequence of a heterologous polypeptide (e.g., a BH3 domain) may be inserted into a scaffold polypeptide (e.g. a SteA scaffold polypeptide) at a site that is amenable to insertion. In some embodiments, an insertion may be a replacement, for example, if an amino acid sequence that forms a loop of a scaffold polypeptide (e.g., loop 1 or loop 2 of SteA or a SteA derivative) is replaced by an amino acid sequence of a heterologous polypeptide.

Insertion Site: As used herein, an “insertion site” is a position or region of a scaffold polypeptide that is amenable to insertion of an amino acid sequence of a heterologous polypeptide. It is to be understood that an insertion site also may refer to the position or region of the polynucleotide that encodes the polypeptide (e.g., a codon of a polynucleotide that codes for a given amino acid in the scaffold polypeptide). In some embodiments, insertion of an amino acid sequence of a heterologous polypeptide into a scaffold polypeptide has little to no effect on the stability (e.g., conformational stability), expression level, or overall secondary structure of the scaffold polypeptide.

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Kozak Sequence: The term “Kozak sequence” (also referred to as “Kozak consensus sequence”) refers to a translation initiation enhancer element to enhance expression of a gene or open reading frame, and which in eukaryotes, is located in the 5′ UTR. The Kozak consensus sequence was originally defined as the sequence GCCRCC, where R=a purine, following an analysis of the effects of single mutations surrounding the initiation codon (AUG) on translation of the preproinsulin gene (Kozak (1986) Cell 44:283-292). Polynucleotides disclosed herein comprise a Kozak consensus sequence, or a derivative or modification thereof. (Examples of translational enhancer compositions and methods of use thereof, see U.S. Pat. No. 5,807,707 to Andrews et al., incorporated herein by reference in its entirety; U.S. Pat. No. 5,723,332 to Chernajovsky, incorporated herein by reference in its entirety; U.S. Pat. No. 5,891,665 to Wilson, incorporated herein by reference in its entirety.)

Leaky scanning: A phenomenon known as “leaky scanning” can occur whereby the PIC bypasses the initiation codon and instead continues scanning downstream until an alternate or alternative initiation codon is recognized. Depending on the frequency of occurrence, the bypass of the initiation codon by the PIC can result in a decrease in translation efficiency. Furthermore, translation from this downstream AUG codon can occur, which will result in the production of an undesired, aberrant translation product that may not be capable of eliciting the desired therapeutic response. In some cases, the aberrant translation product may in fact cause a deleterious response (Kracht et al., (2017) Nat Med 23 (4): 501-507).

Liposome: As used herein, by “liposome” is meant a structure including a lipid-containing membrane enclosing an aqueous interior. Liposomes may have one or more lipid membranes. Liposomes include single-layered liposomes (also known in the art as unilamellar liposomes) and multi-layered liposomes (also known in the art as multilamellar liposomes).

Metastasis: As used herein, the term “metastasis” means the process by which cancer spreads from the place at which it first arose as a primary tumor to distant locations in the body. A secondary tumor that arose as a result of this process may be referred to as “a metastasis.” mRNA: As used herein, an “mRNA” refers to a messenger ribonucleic acid. An mRNA may be naturally or non-naturally occurring. For example, an mRNA may include modified and/or non-naturally occurring components such as one or more nucleobases, nucleosides, nucleotides, or linkers. An mRNA may include a cap structure, a chain terminating nucleoside, a stem loop, a polyA sequence, and/or a polyadenylation signal. An mRNA may have a nucleotide sequence encoding a polypeptide. Translation of an mRNA, for example, in vivo translation of an mRNA inside a mammalian cell, may produce a polypeptide. Traditionally, the basic components of an mRNA molecule include at least a coding region, a 5′-untranslated region (5′-UTR), a 3′UTR, a 5′ cap and a polyA sequence.

microRNA (miRNA): As used herein, a “microRNA (miRNA)” is a small non-coding RNA molecule which may function in post-transcriptional regulation of gene expression (e.g., by RNA silencing, such as by cleavage of the mRNA, destabilization of the mRNA by shortening its polyA tail, and/or by interfering with the efficiency of translation of the mRNA into a polypeptide by a ribosome). A mature miRNA is typically about 22 nucleotides long.

microRNA-122 (miR-122): As used herein, “microRNA-122 (miR-122)” refers to any native miR-122 from any vertebrate source, including, for example, humans, unless otherwise indicated. miR-122 is typically highly expressed in the liver, where it may regulate fatty-acid metabolism. miR-122 levels are reduced in liver cancer, for example, hepatocellular carcinoma. miR-122 is one of the most highly-expressed miRNAs in the liver, where it regulates targets including but not limited to CAT-1, CD320, AldoA, Hjv, Hfe, ADAM10, IGFR1, CCNG1, and ADAM17. Mature human miR-122 may have a sequence of AACGCCAUUAUCACACUAAAUA (SEQ ID NO: 32, corresponding to hsa-miR-122-3p) or UGGAGUGUGACAAUGGUGUUUG (SEQ ID NO: 33, corresponding to hsa-miR-122-5p).

microRNA-21 (miR-21): As used herein, “microRNA-21 (miR-21)” refers to any native miR-21 from any vertebrate source, including, for example, humans, unless otherwise indicated. miR-21 levels are increased in liver cancer, for example, hepatocellular carcinoma, as compared to normal liver. Mature human miR-21 may have a sequence of UAGCUUAUCAGACUGAUGUUGA (SEQ ID NO: 34, corresponding to has-miR-21-5p) or 5′-CAACACCAGUCGAUGGGCUGU-3′ (SEQ ID NO: 35, corresponding to has-miR-21-3p).

microRNA-142 (miR-142): As used herein, “microRNA-142 (miR-142)” refers to any native miR-142 from any vertebrate source, including, for example, humans, unless otherwise indicated. miR-142 is typically highly expressed in myeloid cells. Mature human miR-142 may have a sequence of UGUAGUGUUUCCUACUUUAUGGA (SEQ ID NO: 28, corresponding to hsa-miR-142-3p) or CAUAAAGUAGAAAGCACUACU (SEQ ID NO: 30, corresponding to hsa-miR-142-5p).

microRNA (miRNA) binding site: As used herein, a “microRNA (miRNA) binding site” refers to a miRNA target site or a miRNA recognition site, or any nucleotide sequence to which a miRNA binds or associates. In some embodiments, a miRNA binding site represents a nucleotide location or region of a polynucleotide (e.g., an mRNA) to which at least the “seed” region of a miRNA binds. It should be understood that “binding” may follow traditional Watson-Crick hybridization rules or may reflect any stable association of the miRNA with the target sequence at or adjacent to the microRNA site.

miRNA seed: As used herein, a “seed” region of a miRNA refers to a sequence in the region of positions 2-8 of a mature miRNA, which typically has perfect Watson-Crick complementarity to the miRNA binding site. A miRNA seed may include positions 2-8 or 2-7 of a mature miRNA. In some embodiments, a miRNA seed may comprise 7 nucleotides (e.g., nucleotides 2-8 of a mature miRNA), wherein the seed-complementary site in the corresponding miRNA binding site is flanked by an adenine (A) opposed to miRNA position 1. In some embodiments, a miRNA seed may comprise 6 nucleotides (e.g., nucleotides 2-7 of a mature miRNA), wherein the seed-complementary site in the corresponding miRNA binding site is flanked by an adenine (A) opposed to miRNA position 1. When referring to a miRNA binding site, an miRNA seed sequence is to be understood as having complementarity (e.g., partial, substantial, or complete complementarity) with the seed sequence of the miRNA that binds to the miRNA binding site.

Modified: As used herein “modified” or “modification” refers to a changed state or a change in composition or structure of a polynucleotide (e.g., mRNA) or molecule provided herein. Polynucleotides and molecules may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). In some embodiments, the polynucleotides are modified by the introduction of non-natural nucleosides and/or nucleotides, e.g., as it relates to the natural ribonucleotides A, U, G and C. Noncanonical nucleotides such as the cap structures are not considered “modified” although they differ from the chemical structure of the A, C, G, U ribonucleotides. Accordingly, polynucleotides and molecules of the disclosure may be comprised of one or more modifications (e.g., may include one or more chemical, structural, or functional modifications, including any combination thereof).

Nanoparticle: As used herein, “nanoparticle” refers to a particle having any one structural feature on a scale of less than about 1000 nm that exhibits novel properties as compared to a bulk sample of the same material. Routinely, nanoparticles have any one structural feature on a scale of less than about 500 nm, less than about 200 nm, or about 100 nm. Also routinely, nanoparticles have any one structural feature on a scale of from about 50 nm to about 500 nm, from about 50 nm to about 200 nm or from about 70 to about 120 mn. In exemplary embodiments, a nanoparticle is a particle having one or more dimensions of the order of about 1-1000 nm. In other exemplary embodiments, a nanoparticle is a particle having one or more dimensions of the order of about 10-500 nm. In other exemplary embodiments, a nanoparticle is a particle having one or more dimensions of the order of about 50-200 nm. A spherical nanoparticle would have a diameter, for example, of between about 50-100 or 70-120 nanometers. A nanoparticle most often behaves as a unit in terms of its transport and properties. It is noted that novel properties that differentiate nanoparticles from the corresponding bulk material typically develop at a size scale of under 1000 nm, or at a size of about 100 nm, but nanoparticles can be of a larger size, for example, for particles that are oblong, tubular, and the like. Although the size of most molecules would fit into the above outline, individual molecules are usually not referred to as nanoparticles.

Nucleic acid: As used herein, the term “nucleic acid” is used in its broadest sense and encompasses any compound and/or substance that includes a polymer of nucleotides. These polymers are often referred to as polynucleotides. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof. Furthermore, a nucleic acid may be in the form of a nucleic acid construct, such as a plasmid or a vector (e.g., viral vector, expression vector).

Nucleobase: As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

Nucleoside/Nucleotide: As used herein, the term “nucleoside” refers to a compound containing a sugar molecule (e.g., a ribose in RNA or a deoxyribose in DNA), or derivative or analog thereof, covalently linked to a nucleobase (e.g., a purine or pyrimidine), or a derivative or analog thereof (also referred to herein as “nucleobase”), but lacking an internucleoside linking group (e.g., a phosphate group). As used herein, the term “nucleotide” refers to a nucleoside covalently bonded to an internucleoside linking group (e.g., a phosphate group), or any derivative, analog, or modification thereof that confers improved chemical and/or functional properties (e.g., binding affinity, nuclease resistance, chemical stability) to a nucleic acid or a portion or segment thereof.

Open Reading Frame: As used herein, the term “open reading frame”, abbreviated as “ORF”, refers to a segment or region of an mRNA molecule that encodes a polypeptide. The ORF comprises a continuous stretch of non-overlapping, in-frame codons, beginning with the initiation codon and ending with a stop codon, and is translated by the ribosome.

Patient: As used herein, “patient” refers to a subject who may seek or be in need of treatment, requires treatment, is receiving treatment, will receive treatment, or a subject who is under care by a trained professional for a particular disease or condition. In particular embodiments, a patient is a human patient. In some embodiments, a patient is a patient suffering from cancer (e.g., liver cancer or colorectal cancer).

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio

Pharmaceutically acceptable excipient: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Polypeptide: As used herein, the term “polypeptide” or “polypeptide of interest” refers to a polymer of amino acid residues typically joined by peptide bonds that can be produced naturally (e.g., isolated or purified) or synthetically.

Pre-Initiation Complex (PIC): As used herein, the term “pre-initiation complex” (alternatively “43S pre-initiation complex”; abbreviated as “PIC”) refers to a ribonucleoprotein complex comprising a 40S ribosomal subunit, eukaryotic initiation factors (eIF1, eIF1A, eIF3, eIF5), and the eIF2-GTP-Met-tRNAᵢ^{Met} ternary complex, that is intrinsically capable of attachment to the 5′ cap of an mRNA molecule and, after attachment, of performing ribosome scanning of the 5′ UTR.

RNA element: As used herein, the term “RNA element” refers to a portion, fragment, or segment of an RNA molecule that provides a biological function and/or has biological activity (e.g., translational regulatory activity). Modification of a polynucleotide by the incorporation of one or more RNA elements, such as those described herein, provides one or more desirable functional properties to the modified polynucleotide. RNA elements, as described herein, can be naturally-occurring, non-naturally occurring, synthetic, engineered, or any combination thereof. For example, naturally-occurring RNA elements that provide a regulatory activity include elements found throughout the transcriptomes of viruses, prokaryotic and eukaryotic organisms (e.g., humans). RNA elements in particular eukaryotic mRNAs and translated viral RNAs have been shown to be involved in mediating many functions in cells. Exemplary natural RNA elements include, but are not limited to, translation initiation elements (e.g., internal ribosome entry site (IRES), see Kieft et al., (2001) RNA 7 (2): 194-206), translation enhancer elements (e.g., the APP mRNA translation enhancer element, see Rogers et al., (1999) J Biol Chem 274 (10): 6421-6431), mRNA stability elements (e.g., AU-rich elements (AREs), see Garneau et al., (2007) Nat Rev Mol Cell Biol 8 (2): 113-126), translational repression element (see e.g., Blumer et al., (2002) Mech Dev 110 (1-2): 97-112), protein-binding RNA elements (e.g., iron-responsive element, see Selezneva et al., (2013) J Mol Biol 425 (18): 3301-3310), cytoplasmic polyadenylation elements (Villalba et al., (2011) Curr Opin Genet Dev 21 (4): 452-457), and catalytic RNA elements (e.g., ribozymes, see Scott et al., (2009) Biochim Biophys Acta 1789 (9-10): 634-641).

Residence time: As used herein, the term “residence time” refers to the time of occupancy of a pre-initiation complex (PIC) or a ribosome at a discrete position or location along an mRNA molecule.

Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Suffering from: An individual who is “suffering from” a disease, disorder, and/or condition has been diagnosed with or displays one or more symptoms of a disease, disorder, and/or condition.

Targeting moiety: As used herein, a “targeting moiety” is a compound or agent that may target a nanoparticle to a particular cell, tissue, and/or organ type.

Therapeutic Agent: The term “therapeutic agent” refers to any agent that, when administered to a subject, has a therapeutic, diagnostic, and/or prophylactic effect and/or elicits a desired biological and/or pharmacological effect.

Transfection: As used herein, the term “transfection” refers to methods to introduce a species (e.g., a polynucleotide, such as a mRNA) into a cell.

Translational Regulatory Activity: As used herein, the term “translational regulatory activity” (used interchangeably with “translational regulatory function”) refers to a biological function, mechanism, or process that modulates (e.g., regulates, influences, controls, varies) the activity of the translational apparatus, including the activity of the PIC and/or ribosome. In some aspects, the desired translation regulatory activity promotes and/or enhances the translational fidelity of mRNA translation. In some aspects, the desired translational regulatory activity reduces and/or inhibits leaky scanning. Subject: As used herein, the term “subject” refers to any organism to which a composition in accordance with the disclosure may be administered, e.g., for experimental, diagnostic, prophylactic, and/or therapeutic purposes. Typical subjects include animals (e.g., mammals such as mice, rats, rabbits, non-human primates, and humans) and/or plants. In some embodiments, a subject may be a patient.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Preventing: As used herein, the term “preventing” refers to partially or completely inhibiting the onset of one or more symptoms or features of a particular infection, disease, disorder, and/or condition.

Tumor: As used herein, a “tumor” is an abnormal growth of tissue, whether benign or malignant.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Uridine Content: The terms “uridine content” or “uracil content” are interchangeable and refer to the amount of uracil or uridine present in a certain nucleic acid sequence. Uridine content or uracil content can be expressed as an absolute value (total number of uridine or uracil in the sequence) or relative (uridine or uracil percentage respect to the total number of nucleobases in the nucleic acid sequence).

Uridine-Modified Sequence: The terms “uridine-modified sequence” refers to a sequence optimized nucleic acid (e.g., a synthetic mRNA sequence) with a different overall or local uridine content (higher or lower uridine content) or with different uridine patterns (e.g., gradient distribution or clustering) with respect to the uridine content and/or uridine patterns of a candidate nucleic acid sequence. In the content of the present disclosure, the terms “uridine-modified sequence” and “uracil-modified sequence” are considered equivalent and interchangeable.

A “high uridine codon” is defined as a codon comprising two or three uridines, a “low uridine codon” is defined as a codon comprising one uridine, and a “no uridine codon” is a codon without any uridines. In some embodiments, a uridine-modified sequence comprises substitutions of high uridine codons with low uridine codons, substitutions of high uridine codons with no uridine codons, substitutions of low uridine codons with high uridine codons, substitutions of low uridine codons with no uridine codons, substitution of no uridine codons with low uridine codons, substitutions of no uridine codons with high uridine codons, and combinations thereof. In some embodiments, a high uridine codon can be replaced with another high uridine codon. In some embodiments, a low uridine codon can be replaced with another low uridine codon. In some embodiments, a no uridine codon can be replaced with another no uridine codon. A uridine-modified sequence can be uridine enriched or uridine rarefied.

Uridine Enriched: As used herein, the terms “uridine enriched” and grammatical variants refer to the increase in uridine content (expressed in absolute value or as a percentage value) in a sequence optimized nucleic acid (e.g., a synthetic mRNA sequence) with respect to the uridine content of the corresponding candidate nucleic acid sequence. Uridine enrichment can be implemented by substituting codons in the candidate nucleic acid sequence with synonymous codons containing less uridine nucleobases. Uridine enrichment can be global (i.e., relative to the entire length of a candidate nucleic acid sequence) or local (i.e., relative to a subsequence or region of a candidate nucleic acid sequence).

Uridine Rarefied: As used herein, the terms “uridine rarefied” and grammatical variants refer to a decrease in uridine content (expressed in absolute value or as a percentage value) in an sequence optimized nucleic acid (e.g., a synthetic mRNA sequence) with respect to the uridine content of the corresponding candidate nucleic acid sequence. Uridine rarefication can be implemented by substituting codons in the candidate nucleic acid sequence with synonymous codons containing less uridine nucleobases. Uridine rarefication can be global (i.e., relative to the entire length of a candidate nucleic acid sequence) or local (i.e., relative to a subsequence or region of a candidate nucleic acid sequence).

### Equivalents and Scope

Those skilled in the art will recognize, or be able to ascertain using no more than routine experimentation, many equivalents to the specific embodiments in accordance with the disclosure described herein. The scope of the present disclosure is not intended to be limited to the Description below, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the disclosure, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

### EXAMPLES

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### Example 1: STING Immune Potentiator mRNA Constructs

In this example, a series of mmRNA constructs that encoded constitutively activated forms of human STING were made and tested for their ability to stimulate interferon-β (IFN-β) production. The human STING protein encoded by the constructs was constitutively activated through introduction of one or more point mutations. The following single or combination point mutations were tested: (i) V155M; (ii) R284T; (iii) V147L/N154S/V155M; and (iv) R284M/V147L/N154S/V155M. These constructs typically also encoded an epitope tag at either the N-terminus or C-terminus to facilitate detection. Different epitope tags were tested (FLAG, Myc, CT, HA, V5). Additionally, all constructs contained a Cap 15′ Cap (7mG(5′)ppp(5′)NlmpNp), 5′ UTR, 3′ UTR, a poly A tail of 100 nucleotides and were fully modified with 1-methyl-pseudouridine (m1ψ). The ORF amino acid sequences of representative constitutively active human STING constructs without any epitope tag are shown in SEQ ID NOs: 1-10. Exemplary nucleotide sequences encoding these amino acid sequences are shown in SEQ ID NOs: 199-208 and 1442-1450. Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

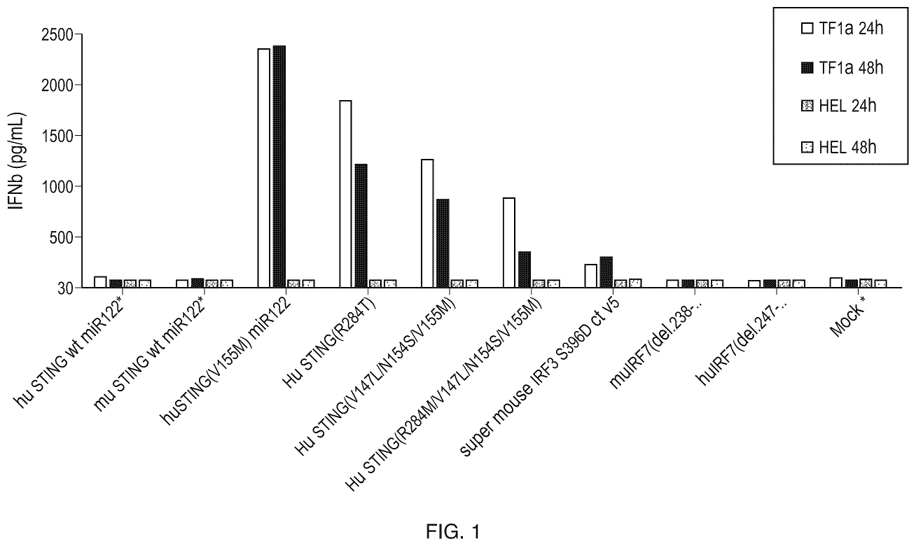

To determine whether constitutively active STING constructs could stimulate IFN-β production, the constructs were transfected into human TF1a cells. Wild-type (non-constitutively active) human and mouse STING constructs were used as negative controls. Twenty-five thousand cells/well were plated in 96 well plates and the mmRNA constructs (250 ng) were transfected into them using Lipofectamine 2000. After 24 and 48 hours, supernatants were harvested and IFN-β levels were determined by standard ELISA. The results are shown in FIG. 1, which demonstrate that the constitutively active STING constructs stimulated IFN-β production, as compared to the wild-type (non-constitutively active) human and mouse STING controls. While all four mutant STING constructs stimulated IFN-production, the V155M mutant and the R284T mutant showed the highest activity. These results demonstrate the ability of constitutively active STING mRNA constructs to enhance immune responses through stimulation of IFN-β production.

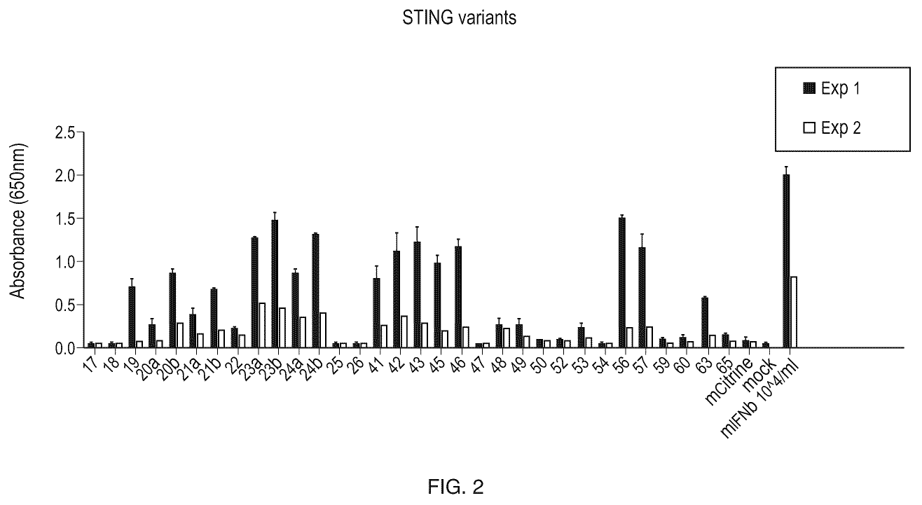

In a second set of experiments, a reporter gene whose transcription was driven by an interferon-sensitive response element (ISRE) was used to test the ability of a panel of constitutively active STING mRNA constructs to activate the ISRE in a STING KO reporter mouse cell line derived from B16 melanocytes. The results are shown in FIG. 2, which demonstrates that the constitutively active STING constructs stimulated reporter gene expression, thereby indicating that the constructs were capable of activating the interferon-sensitive response element (ISRE).

### Example 2: IRF3 and IRF7 Immune Potentiator mRNA Constructs

In this example, a series of mmRNA constructs that encoded constitutively activated forms of IRF3 or IRF7 were made and tested for their ability to activate an interferon-sensitive response element (ISRE). The ORF amino acid sequences of representative constitutively active mouse and human IRF3 constructs, comprising a S396D point mutation, without any epitope tag are shown in SEQ ID NOs: 11-12. Exemplary nucleotide sequences encoding these amino acid sequences are shown in SEQ ID NOs: 210-211. The ORF amino acid sequence of a wild-type human IRF7 construct without any epitope tag is shown in SEQ ID NO: 13 (encoded by the nucleotide sequence shown in SEQ ID NO: 212). The ORF amino acid sequences of representative constitutively active human IRF7 constructs without any epitope tag are shown in SEQ ID NOs: 14-18. Exemplary nucleotide sequences encoding these amino acid sequences are shown in SEQ ID NOs: 213-217 and 142-1459. The ORF amino acid sequences of representative truncated human IRF7 constructs (inactive “null” mutations) without any epitope tag are shown in SEQ ID NOs: 19-20. Exemplary nucleotide sequences encoding these amino acid sequences are shown in SEQ ID NOs: 218-219. These constructs typically also encoded an epitope tag at either the N-terminus or C-terminus to facilitate detection. Different epitope tags were tested (FLAG, Myc, CT, HA, V5). Additionally, all constructs contained a Cap 1 5′ Cap (7 mG (5′)ppp(5′) NlmpNp), 5′ UTR, 3′ UTR, a poly A tail of 100 nucleotides and were fully modified with 1-methyl-pseudouridine (m1ψ). Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

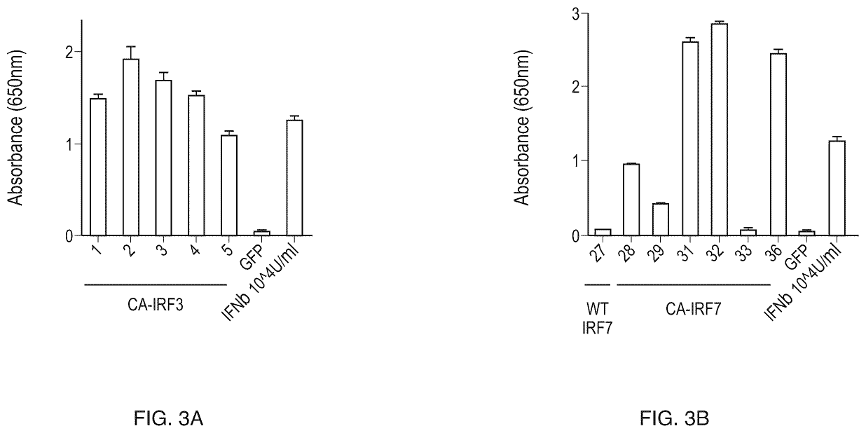

The reporter cell line used in Example 1, whose transcription was driven by an interferon-sensitive response element (ISRE), was used to test the ability of constitutively active IRF3 and IRF7 mRNA constructs to activate the ISRE. The results are shown in FIG. 3A-3B, which demonstrate that the constitutively active IRF3 constructs (FIG. 3A) and the constitutively active IRF7 constructs (FIG. 3B) stimulated reporter gene expression, thereby indicating that the constructs were capable of activating the interferon-sensitive response element (ISRE).

### Example 3: IKKβ, cFLIP and RIPK1 Immune Potentiator mRNA Constructs

In this example, a luciferase reporter gene whose transcription was driven by the NFκB signaling pathway was used to test the ability of constitutively active IKK, cFLIP and RIPK1 mRNA constructs to activate NFκB signaling.

Constitutively active IKKβ construct comprised the following two point mutations: S177E/S181E. Constitutively active IKKα or IKKβ constructs comprised PEST mutations. The ORF amino acid sequences of constitutively active IKKβ constructs without any epitope tag are shown in SEQ ID NOs: 146-149. Exemplary nucleotide sequences encoding the protein of SEQ ID NO: 146 are shown in SEQ ID NOs: 1414 and 1485. The ORF amino acid sequences of constitutively active IKKα or IKKβ constructs comprising a PEST mutation, without any epitope tag, are shown in SEQ ID NOs: 150, 152, 154 and 156 (encoded by the nucleotide sequences shown in SEQ ID NOs: 151, 153, 155 and 157, respectively, or SEQ ID NO NOs. 1428, 1397, 1429 and 1430, respectively). Constitutively active cFLIP constructs comprised cFLIP-L, cFLIP-S(aa 1-227), cFLIP p22 (aa 1-198), cFLIP p43 (aa 1-376) or cFLIP p12 (aa 377-480). The ORF amino acid sequences of the cFLIP constructs without any epitope tag are shown in SEQ ID NOs: 141-145. Exemplary nucleotide sequences encoding these cFLIP proteins are shown in SEQ ID NOs: 1398-1402 and 1469-1473. Structures of various constitutively active RIPK1 constructs are described further in, for example, Yatim, N. et al. (2015) Science 350:328-334 or Orozco, S. et al. (2014) Cell Death Differ. 21:1511-1521. The ORF amino acid sequences of the constitutively active RIPK1 constructs without any epitope tag are shown in SEQ ID NOs: 158-163. Exemplary nucleotide sequences encoding these RIPK1 proteins are shown in SEQ ID NOs: 1403-1408 and 1474-1479. In addition to the open reading frame, all constructs contained a Cap 15′ Cap (7 mG (5′)ppp(5′) NlmpNp), 5′ UTR, 3′ UTR, a poly A tail of 100 nucleotides and were fully modified with 1-methyl-pseudouridine (m1ψ). Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

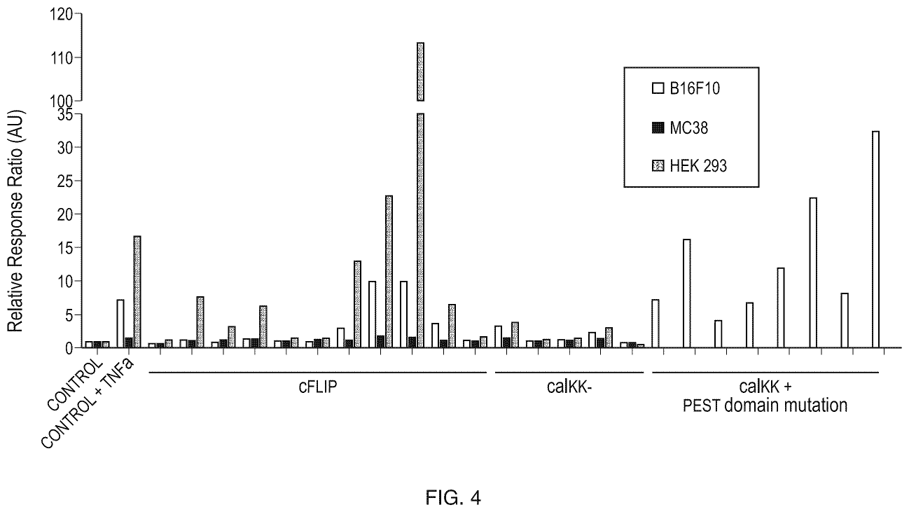

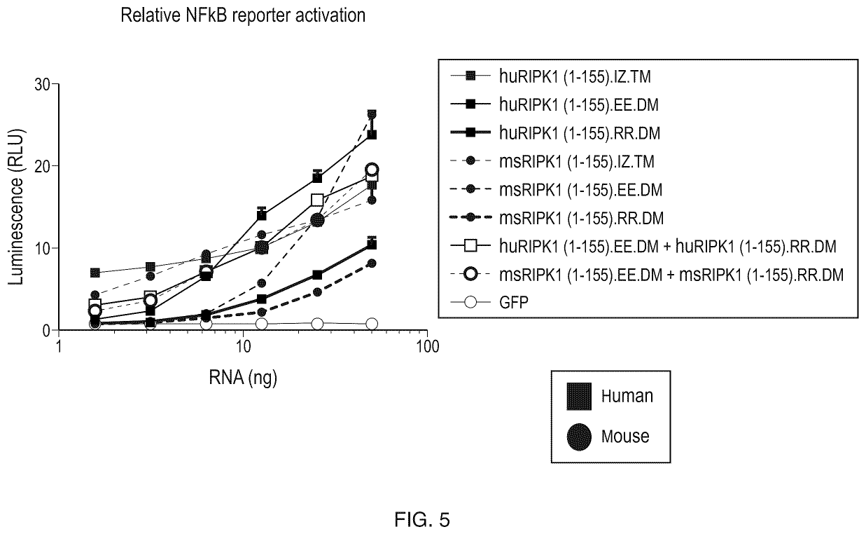

In a first series of experiments, either the cFLIP or IKKβ constructs (12.5 ng RNA) were transfected into B16F10, MC38 or HEK293 cells, together with the NFκB-luc reporter gene and the Dual Luc Assay was performed 24 hours post-transfection as an indicator of activation of NFκB signaling. The results are shown in FIG. 4, which demonstrates that the constitutively active cFLIP and IKKβ constructs stimulated reporter gene expression, thereby indicating that the constructs were capable of activating the NFκB signaling pathway. In a second series of experiments, the RIPK1 constructs were transfected into B16F10 cells, together with the NFκB-luc reporter gene and the Dual Luc Assay was performed 24 hours post-transfection as an indicator of activation of NFκB signaling. The results are shown in FIG. 5, which demonstrates that the constitutively active RIPK1 constructs stimulated reporter gene expression, thereby indicating that the constructs were capable of activating the NFκB signaling pathway.

### Example 4: DIABLO Immune Potentiator mRNA Constructs

In this example, a series of mmRNA constructs that encoded DIABLO were made and tested for their ability to induce cytokine production. These constructs typically also encoded an epitope tag at either the N-terminus or C-terminus to facilitate detection. Different epitope tags were tested (FLAG, Myc, CT, HA, V5). Additionally, all constructs contained a Cap 1 5′ Cap (7mG (5′)ppp(5′) NlmpNp), 5′ UTR, 3′ UTR, a poly A tail of 100 nucleotides and were fully modified with 1-methyl-pseudouridine (m1ψ). The ORF amino acid sequences of the DIABLO constructs without any epitope tag are shown in SEQ ID NOs: 165-172. Exemplary nucleotide sequences encoding the DIABLO protein of SEQ ID NO: 169 is shown in SEQ ID NOs: 1416 and 1487. Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

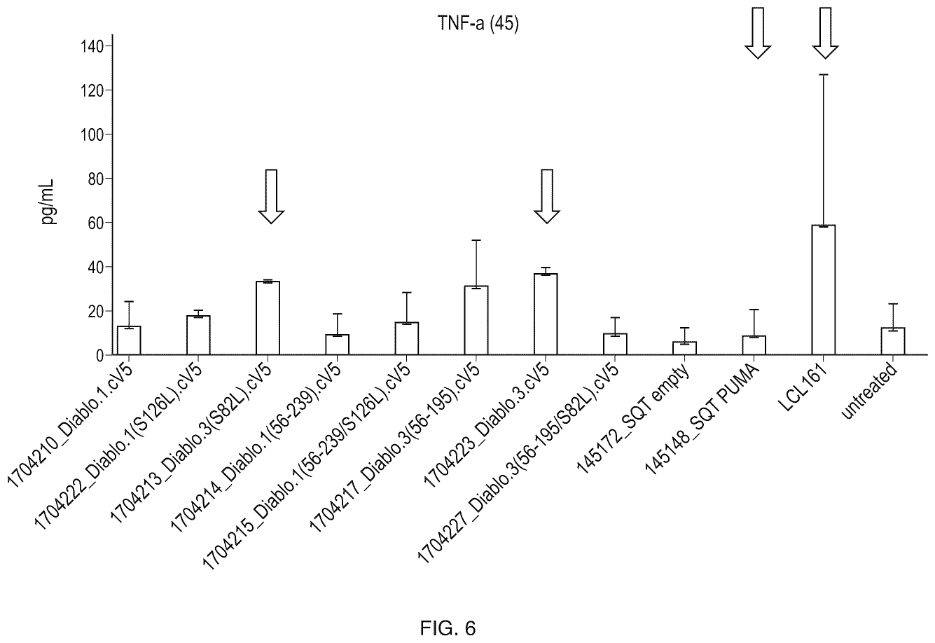

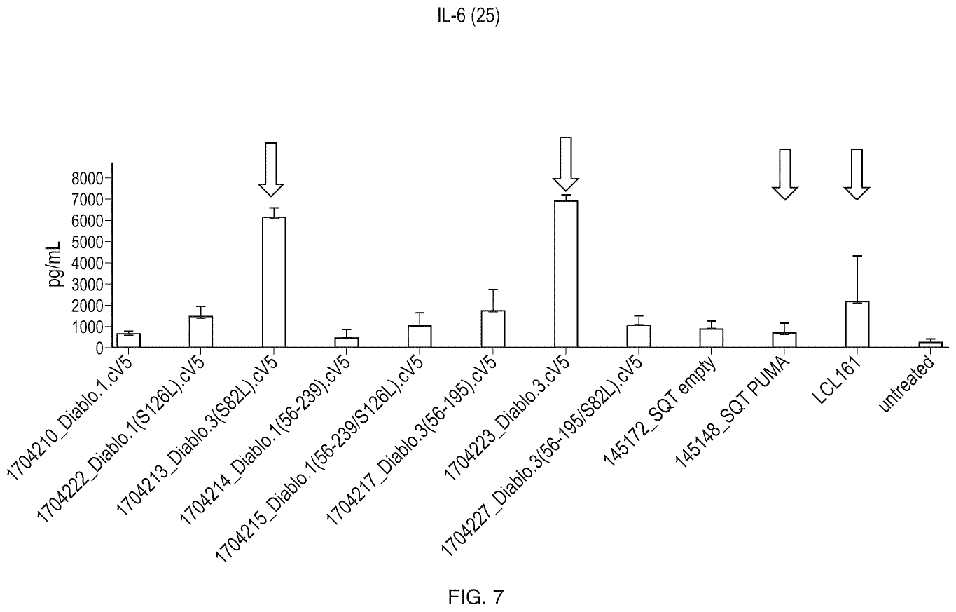

To determine whether the DIABLO constructs could induce cytokine production, the constructs were transfected into SKOV3 cells. Ten thousand cells/well were plated in 96 well plates and the mmRNA constructs were transfected into them using Lipofectamine 2000. Stimulation of cytokine production by the DIABLO mmRNA constructs in the SKOV3 cells was measured. The results, shown in FIG. 6 for TNF-α and in FIG. 7 for interleukin 6 (IL-6), demonstrate that a number of the DIABLO mmRNA constructs stimulate production of cytokines by the SKOV3 cells.

### Example 5: Immune Potentiator mRNAs Enhance HPV Vaccine Responses

In this example, the potency and durability of responses to a human papillomavirus (HPV) E6/E7 mRNA-based vaccine used in combination with STING, IRF3 or IRF7 immune potentiators were examined. A specific immune response to human papillomavirus (HPV) in the cervical microenvironment is known to play a key role in eradicating infection and eliminating mutated cells. However, high-risk HPVs are known to modulate immune cells to create an immunosuppressive microenvironment (see e.g., Prata, T. T. et al. (2015) Immunology 146:113-121). Thus, an HPV vaccination approach that leads to a robust and durable immune response is highly desirable.

The HPV vaccines used in this example were mRNA constructs encoding either intracellular or soluble forms of HPV 16 antigens E6 and E7, referred to herein as iE6/E7 and sE6/E7, respectively. To create the soluble format, a signal peptide required for secretion was fused to the N-terminal of the antigen. The sequence of the signal peptide was derived from the Ig kappa chain V-III region HAH. Mice were immunized intramuscularly with either the iE6/E7 or sE6/E7 mRNA vaccine (at a dose of 0.25 mg/kg) on days 0 and 14, in combination with either a control mRNA construct (NTFIX), or a STING, IRF3 or IRF7 immune potentiator mRNA construct (at a dose of 0.25 mg/kg). The constitutively active STING immune potentiator contained a V155M mutation (mouse version corresponding to SEQ ID NO: 1). The constitutively active IRF3 immune potentiator contained a S396D mutation (corresponding to SEQ ID NO: 12). The constitutively active IRF7 immune potentiator contained an internal deletion and six point mutations (mouse version corresponding to SEQ ID NO: 18). The HPV vaccine construct and the immune potentiator construct were coformulated in MC3 lipid nanoparticles.

At day 21 and 53, spleen cells and peripheral blood mononuclear cells (PBMC) from mice in each test group were restimulated ex vivo for 4 hours at 37 degrees C. in the presence of GolgiPlug™ (containing Brefeldin A; BD Biosciences) with either: an E6 peptide pool (containing 37 E6 peptides, the sequences of which are shown in SEQ ID NOs: 36-72), an E7 peptide pool (containing 22 E7 peptides, the sequences of which are shown in SEQ ID NOs: 73-94), E6 single peptides (8 individual peptides), E7 single peptides (7 individual peptides) or no peptides (control). Each peptide was provided at a dose of 0.2 μg/ml. CD8 vaccine responses were assessed by intracellular staining (ICS) for IFN-γ or TNF-α.

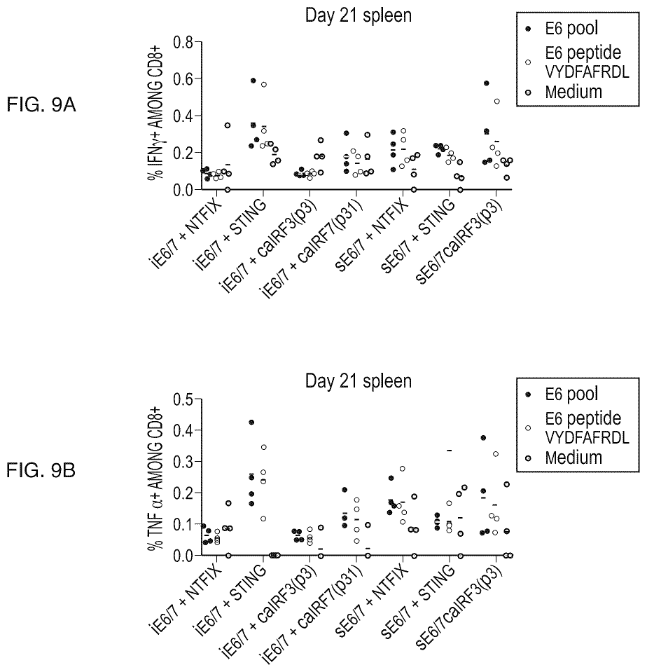

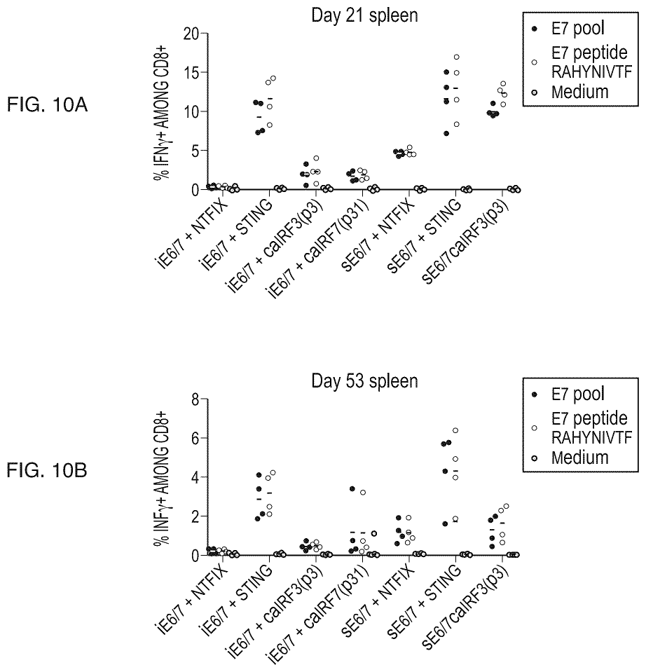

Representative ICS results for E7-specific responses by day 21 spleen cells for IFN-γ and TNF-α are shown in FIG. 8A (IFN-γ) and FIG. 8B (TNF-α). Representative ICS results for E6-specific responses by day 21 spleen cells for IFN-γ and TNF-α are shown in FIG. 9A (IFN-γ) and FIG. 9B (TNF-α). The results in FIGS. 8A-8B and 9A-9B demonstrate that CD8 vaccine responses (to both the intracellular and soluble antigen format) were greatly enhanced when the STING, IRF3 or IRF7 immune potentiators were co-formulated with the vaccine, with the E7 epitope being stronger and less variable than the E6 epitope and with the soluble form of antigen being stronger than the intracellular form of antigen. This enhanced CD8 vaccine responses by the immune potentiators was shown to be durable, as evidenced by the representative day 21 versus day 53 E7-specific spleen cell IFN-γ ICS data shown in FIGS. 10A and 10B, respectively. Similar results to the spleen cell data were observed for the PBMC experiments (data not shown). The ability of STING to improve the durability of antigen-specific CD8 responses is further demonstrated by the IFN-γ ICS data shown in FIG. 11A (E7-specific responses from mice immunized with intracellular E6/E7) and FIG. 11B (E7-specific responses from mice immunized with soluble E6/E7), in which over the course of the 7 week experiment it was demonstrated that a higher percentage of antigen-specific CD8 T cells were maintained in the STING-treated mice.

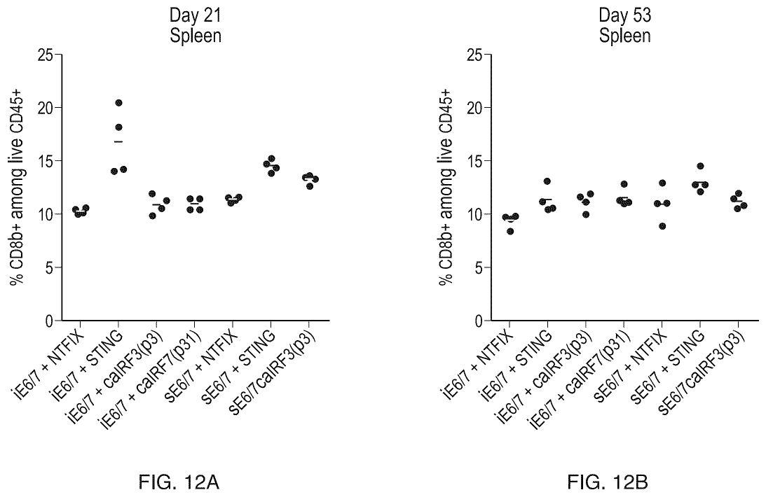

The percentage of CD8b⁺ cells among the live CD45⁺ cells was also examined. The results for day 21 versus day 53 spleen cells are shown in FIGS. 12A and 12B, respectively. The results demonstrate that the immune potentiators (in particular the STING construct) expand the total CD8b⁺ population on day 21 but not day 53.

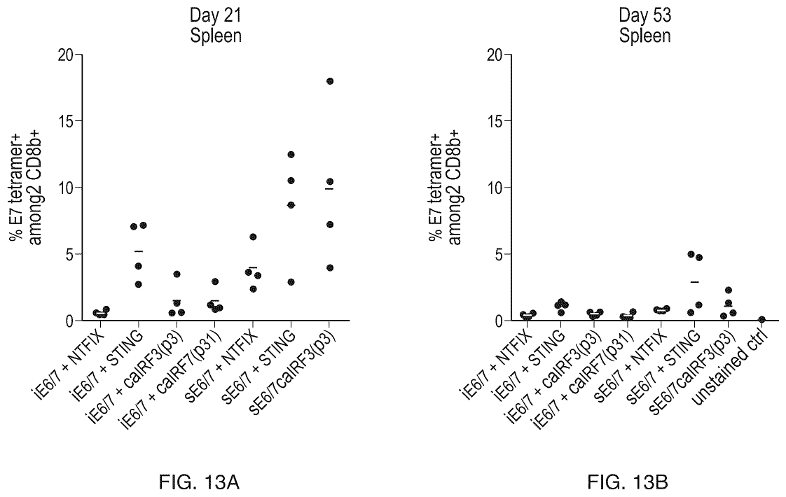

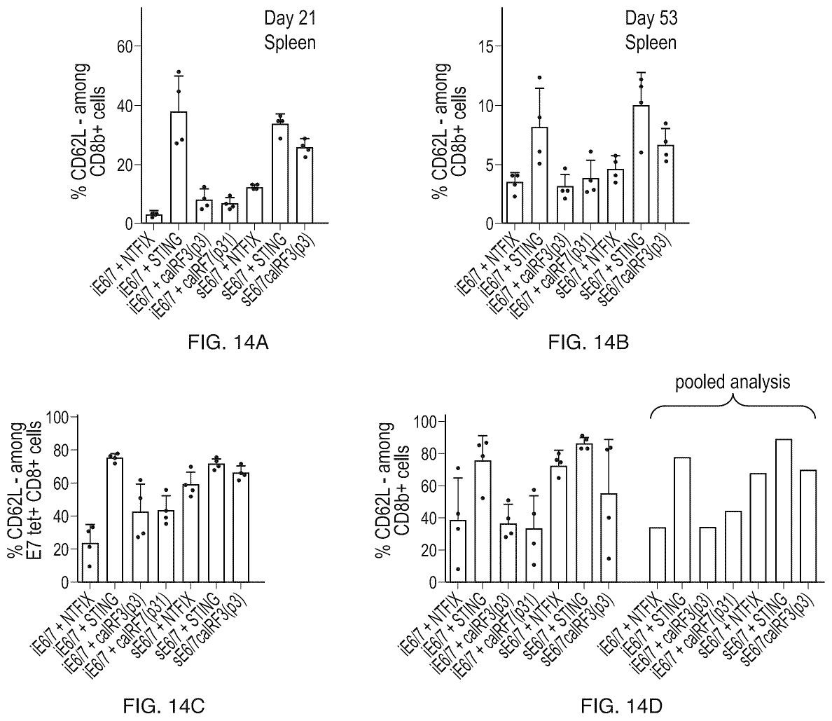

The ability of the immune potentiator constructs to enhance the CD8 vaccine response was further confirmed by E7-MHC1-tetramer staining. Representative results for day 21 versus day 53 spleen cells are shown in FIGS. 13A and 13B, respectively. The E7-MHC-1-tetramer staining results were consistent with the ICS results discussed above, although they were more variable. As demonstrated in FIGS. 14A-14D, the majority of the tetramer positive CD8 cells were found to have an “effector memory” CD62L^{lo} phenotype. Comparison of day 21 versus day 53 E7-tetramer+CD8 cells demonstrated that this “effector-memory” CD62L^{lo} phenotype was maintained throughout the study. Additional staining experiments demonstrated that the immune potentiators slightly reduced the % of total Foxp3⁺ Treg CD4 T cells (data not shown) and did not change the % of CD138⁺ plasmablasts (data not shown).

### Example 6: Immune Potentiator mRNAs Enhance MC38 Cancer Vaccine Responses

In this example, the potency and durability of responses to an MC38 mRNA-based cancer vaccine used in combination with STING, IRF3 or IRF7 immune potentiator mRNA constructs were examined. The MC38 murine tumor model has been used to identify immunogenic mutant peptides containing neoepitopes capable of stimulating anti-tumor T cell responses (see e.g., Yadav, M. et al. (2014) Nature 515:572-576). Thus, a cancer vaccination approach that leads to a robust and durable immune response against tumor neoepitopes is highly desirable.

The MC38 vaccine used in this example was an mRNA construct encoding an ADR concatemer of three 25mer mutant peptides containing tumor neoepitopes derived from Adpgk, Dpagt1, and Reps1 (this vaccine is also referred to herein as ADRvax). The mRNA construct encodes the open reading frame shown in SEQ ID NO: 179, which also includes an N-terminal His-tag for easy detection. Mice were immunized intramuscularly with the ADRvax mRNA vaccine (at a dose of 0.25 mg/kg) on days 0 and 14, in combination with either a control mRNA construct (NTFIX), or a STING, IRF3 or IRF7 immune potentiator mRNA construct (at a dose of 0.25 mg/kg). The constitutively active STING immune potentiator contained a V155M mutation (mouse version corresponding to SEQ ID NO: 1). The constitutively active IRF3 immune potentiator contained a S396D mutation (corresponding to SEQ ID NO: 12). The constitutively active IRF7 immune potentiator contained an internal deletion and six point mutations (mouse version corresponding to SEQ ID NO: 18). The MC38 vaccine construct and the immune potentiator construct were coformulated in MC3 lipid nanoparticles.

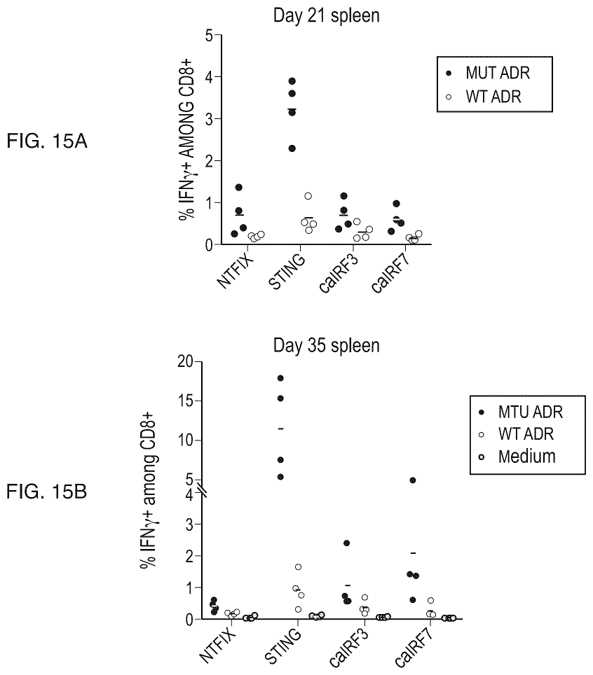

At day 21 and 35, CD8⁺ spleen cells from mice in each test group were restimulated ex vivo for 4 hours at 37 degrees C. in the presence of GolgiPlug™ (containing Brefeldin A; BD Biosciences) with either wild-type or mutant MC38 ADR peptides (1 μg/ml per peptide) and CD8 vaccine responses were assessed by intracellular staining (ICS) for IFN-γ. Representative ICS results for MC38 ADR-specific responses by day 21 and day 35 CD8⁺ spleen cells for IFN-γ are shown in FIG. 15A (day 21) and FIG. 15B (day 35). Similar results were observed for ICS for TNF-α and for CD8⁺PBMCs. The results demonstrate that CD8 vaccine responses were greatly enhanced by the STING immune potentiator construct, and moderately enhanced by the IRF3 and IRF7 immune potentiator constructs. An initial improvement in the antigen-specific CD8 response for mice treated with immune potentiators was observed at day 21 (approximately 5% versus 1% for STING treatment vs. control), which continued to improve by day 35 (up to 15% for STING treatment compared to control), thereby demonstrating the durability of the response.

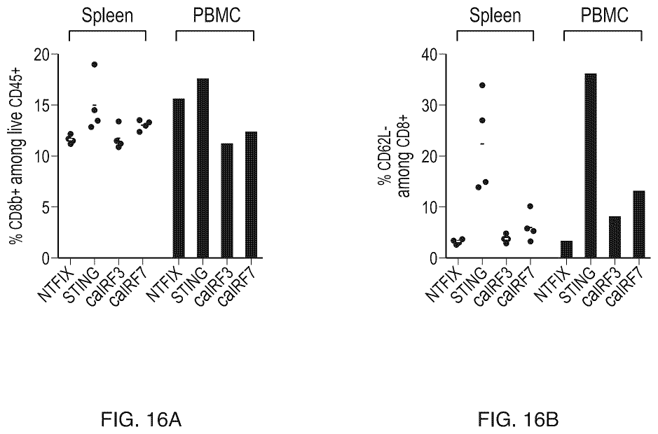

The percentage of CD8b⁺ cells among the live CD45⁺ cells was also examined. The results for day 35 spleen cells and PBMCs are shown in FIG. 16A, which demonstrates that the immune potentiator constructs expand the total CD8b⁺ population. As demonstrated in FIG. 16B, the majority of the CD8⁺ spleen cells and PBMCs were found to have an “effector memory” CD62L^{lo} phenotype. Additional staining experiments demonstrated that the STING and IRF7 immune potentiator construct slightly reduced the % of total Foxp3⁺ Treg CD4 T cells (data not shown). Additional staining experiments demonstrated that the immune potentiators did not change the % of CD138⁺ plasmablasts (data not shown).

### Example 7: Immune Potentiator mRNAs Enhance Bacterial Vaccine Responses

In this example, the potency of responses to a bacterial mRNA-based vaccine used in combination with a STING immune potentiator was examined, in particular the effect of the immune potentiator on the humoral immune response (antibody production) against the bacterial antigens.

The bacterial vaccine used in this example was a pool of mRNA constructs encoding a panel of bacterial antigenic peptides that had been established in the art to provide protective immunity against bacterial infection. Thus, the vaccine used in this example was a multivalent mRNA-based bacterial vaccine. The bacterial peptide antigen mRNA constructs encoded the ORF for the peptide antigens and also contained a Cap 1 5′ Cap (7 mG (5′)ppp(5′) NlmpNp), 5′ UTR, 3′ UTR, a poly A tail of 100 nucleotides and were fully modified with 1-methyl-pseudouridine (m1ψ). Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23. These constructs optionally also can encode an epitope tag at either the N-terminus or C-terminus to facilitate detection. Different epitope tags were tested (FLAG, Myc, CT, HA, V5).

The bacterial peptide antigen mRNA constructs were administered to mice at a dose of 0.2 μg or 0.8 μg per antigen on day 0, 14 and 28, either alone or in combination with a STING immune potentiator mRNA construct. The constitutively active STING immune potentiator contained a V155M mutation (mouse version corresponding to SEQ ID NO: 1). Serum was harvested pre-treatment and at days 14, 28 and 35. Antibody titers were compared between the mice treated with the bacterial peptide antigen mRNA constructs alone versus those treated with the bacterial peptide antigen mRNA constructs in combination with a STING mRNA construct. Mice treated with the higher dose (0.8 μg) of the bacterial antigen peptide mRNA constructs showed a modest effect on antigen-specific antibody titers by co-treatment with the STING construct (data not shown). However, as shown in FIG. 17, co-treatment with the STING construct showed a significant effect on antigen-specific antibody titers in mice treated with the lower dose (0.2 μg) of the bacterial peptide antigen mRNA constructs (referred to as RNAs 0298, 2753, 2723, 2635, 1507, 0992 and 0735 in FIG. 17). These results demonstrate that the STING immune potentiator enhanced the humoral immune response against the bacterial peptide antigens encoded by the bacterial mRNA vaccine constructs, particularly when the bacterial mRNA vaccine construct was used at lower doses.

### Example 8: KRAS-STING mRNA Constructs

A comprehensive survey of Ras mutations in various cancer types has been reported (Prior, I. A. et al. (2012) Cancer Res. 72:2457-2467). This survey demonstrated that the top three most frequent mutations of KRAS in colorectal cancer are G12D, G12V and G13D. A series of mutant KRAS mRNA constructs were prepared that encoded one or more KRAS peptides containing one of these three mutations, for use as KRAS anti-tumor mRNA-based vaccines. Furthermore, to examine the effect of mRNA-based immune potentiators on KRAS vaccine responses, a series of mRNA constructs were prepared that encoded one or more mutant KRAS peptides linked at the N-terminus or the C-terminus to sequence encoding STING as an immune potentiator. Thus, in these KRAS-STING mRNA constructs, the vaccine antigen(s) and the immune potentiator are encoded by the same mRNA construct.

Mutant KRAS peptide mRNA constructs were prepared that encoded: a 15mer peptide having the G12D, G12V or the G13D mutation (the amino acid sequence of which is shown in SEQ ID NOs: 95-97, respectively); a 25mer peptide having the G12D, G12V or the G13D mutation (SEQ ID NOs: 98-100, respectively); three copies of the 15mer peptide having the G12D, G12V or the G13D mutation (SEQ ID NOs: 101-103, respectively); or three copies of the 25mer peptide having the G12D, G12V or the G13D mutation (SEQ ID NOs: 104-106, respectively). Additional constructs encoded one copy or three copies of a 25mer peptide having a G12C mutation (SEQ ID NOs: 131-132, respectively) or a wild-type 25mer peptide (SEQ ID NO: 133). In certain embodiments, a G12C KRAS mutation may be used in combination with a G12D, G12V or G13D mutation, or combinations thereof. Nucleotide sequences encoding these mutant KRAS peptides are provided in Example 9.

Mutant KRAS peptide-STING mRNA constructs, having the STING coding sequence at the N-terminus, were prepared that encoded: a 15mer peptide having the G12D, G12V or the G13D mutation (the amino acid sequence of which is shown in SEQ ID NOs: 107-109, respectively); a 25mer peptide having the G12D, G12V or the G13D mutation (SEQ ID NOs: 110-112, respectively); three copies of the 15mer peptide having the G12D, G12V or the G13D mutation (SEQ ID NOs: 113-115, respectively); or three copies of the 25mer peptide having the G12D, G12V or the G13D mutation (SEQ ID NOs: 116-118, respectively). In certain embodiments, a G12C KRAS mutation may be used in combination with a G12D, G12V or G13D mutation, or combinations thereof. Representative nucleotide sequences encoding these KRAS peptide-STING constructs having the STING coding sequence at the N-terminus are shown in SEQ ID NOs: 220 and 222.

Mutant KRAS peptide-STING mRNA constructs, having the STING coding sequence at the C-terminus, were prepared that encoded: a 15mer peptide having the G12D, G12V or the G13D mutation (the amino acid sequence of which is shown in SEQ ID NOs: 119-121, respectively); a 25mer peptide having the G12D, G12V or the G13D mutation (SEQ ID NOs: 122-124, respectively); three copies of the 15mer peptide having the G12D, G12V or the G13D mutation (SEQ ID NOs: 125-127, respectively); or three copies of the 25mer peptide having the G12D, G12V or the G13D mutation (SEQ ID NOs: 128-130, respectively). In certain embodiments, a G12C KRAS mutation may be used in combination with a G12D, G12V or G13D mutation, or combinations thereof. Representative nucleotide sequences encoding these KRAS peptide-STING constructs having the STING coding sequence at the C-terminus are shown in SEQ ID NOs: 221 and 223.

These constructs can also encode an epitope tag at either the N-terminus or C-terminus to facilitate detection. Different epitope tags can be used (e.g., FLAG, Myc, CT, HA, V5). Additionally, all constructs contained a Cap 15′ Cap (7 mG (5′)ppp(5′) NlmpNp), 5′ UTR, 3′ UTR, a poly A tail and were fully modified with 1-methyl-pseudouridine (m1ψ). Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

To test vaccine responses in mice treated either with a KRAS mutant peptide(s) mRNA vaccine construct or with a KRAS mutant peptide(s) vaccine-STING immune potentiator mRNA construct, mice (HLA-A*11:01 or HLA-A*2:01; Taconic) are treated with a KRAS mutant peptide vaccine mRNA construct (e.g., encoding one of SEQ ID NOs: 95-106) or with a KRAS mutant peptide vaccine-STING immune potentiator mRNA construct (e.g., encoding one of SEQ ID NOs: 107-130). Mice are immunized intramuscularly on day 1 and day 15 (0.5 mg/kg) and sacrificed at day 22. To test CD8 vaccine responses, CD8⁺ spleen cells and PBMCs are restimulated ex vivo for 4 hours at 37 degrees C. in the presence of GolgiPlug™ (containing Brefeldin A; BD Biosciences) with either mutant KRAS peptides (G12D, G12V or G13D) or with wild type KRAS peptide (1 μg/ml per peptide). CD8 vaccine responses can then be assessed by intracellular staining (ICS) for IFN-γ and/or TNF-α. Enhanced ICS responses for IFN-γ and/or TNF-α in mice treated with the KRAS mutant peptide vaccine-STING immune potentiator mRNA construct, as compared to treatment with the KRAS mutant peptide vaccine mRNA construct, indicates that the STING immune potentiator enhances KRAS-specific CD8 vaccine responses.

### Example 9: Use of Immune Potentiator mRNA Construct in Combination with Activating Oncogene KRAS Mutant Peptide mRNA Constructs

In this example, mutant KRAS peptide mRNA constructs are used in combination with a separate constitutively active STING immune potentiator mRNA construct to enhance immune responses to the mutant KRAS peptides.

KRAS is the most frequently mutated oncogene in human cancer (˜15%). KRAS mutations occur mostly in a couple of “hotspots” and activate the oncogene. Prior research has shown limited ability to raise T cells specific to the oncogenic mutation. However, much of this was done in the context of the most common HLA allele (A2, which occurs in ˜50% of Caucasians). More recently, it has been demonstrated that (a) specific T cells can be generated against point mutations in the context of less common HLA alleles (A11, C₈), and (b) growing these cells ex-vivo and transferring them back to the patient has mediated a dramatic tumor response in a patient with lung cancer. (N Engl J Med 2016; 375:2255-2262 Dec. 8, 2016DOI: 10.1056/NEJMoa1609279).

As shown in Table 5 below, in CRC (colorectal cancer), only 3 mutations (G12V, G12D, and G13D) account for 96% of KRAS mutations in this malignancy. Furthermore, all CRC patients get typed for KRAS mutations as standard of care.

TABLE 5
COSMIC* case counts

| | All cancers | % | CRC | % |
|---|---|---|---|---|
| G12S | 1849 | 1% | | |
| G12V | 9213 | 4% | 5215 | 29% |
| G12C | 435 | 2% | | |
| G12D | 13634 | 7% | 8083 | 44% |
| G12A | 2179 | 1% | | |
| G12R | 1244 | 1% | | |
| G13D | 5084 | 2% | 4267 | 23% |
| | | 18% | | 96% |
| Tested | 208629 | | 18271 | |

*http://cancer.sanger.ac.uk/cosmic/gene/analysis?In=KRAS

In another COSMIC data set, 73.68% of KRAS mutations in colorectal cancer are accounted for by these 3 mutations (G12V, G12D, and G13D) (Table 6).

TABLE 6

| | colon | % | rectal | % | total | % |
|---|---|---|---|---|---|---|
| 12D | 635 | 35.04 | 178 | 33.46 | 813 | 34.68 |
| 12V | 364 | 20.09 | 124 | 23.31 | 488 | 20.82 |
| 13D | 338 | 18.65 | 88 | 16.54 | 426 | 18.17 |
| | | | | | | 73.68 |

Prior et al. investigated and summarized isoform-specific point mutation specificity for HRAS, KRAS, and NRAS, respectively (Prior et al. Cancer Res. 2012 May 15; 72 (10): 2457-2467). Data representing total number of tumors with each point mutation were collated from COSMIC v52 release. The most frequent mutations for each isoform for each cancer type are reported (see Table 2 of Prior et al.).

In addition, secondary KRAS mutations have been identified in EGFR blockade resistant patients. RAS is downstream of EGFR and it has been found to constitute a mechanism of resistance to EGFR blockade therapies. EGFR blockade resistant KRAS mutant tumors can be targeted using compositions and methods disclosed herein. In a few cases, more than one KRAS mutation was identified in the same patient (up to four different mutations co-occur). Diaz et al. reports these secondary KRAS mutations after acquisition of EGFR blockade (see Supplementary Table 2), and Misale et al. reports secondary KRAS mutations after EGFR blockade (see FIG. 3b) (Diaz et al. The molecular evolution of acquired resistance to targeted EGFR blockade in colorectal cancers, Nature 486:537 (2012); Misale et al. Emergence of KRAS mutations and acquired resistance to anti-EGFR therapy in colorectal cancer, Nature 486:532 (2012)). This mutational spectrum appears to be at least somewhat different than primary tumor missense mutants in colorectal cancer.

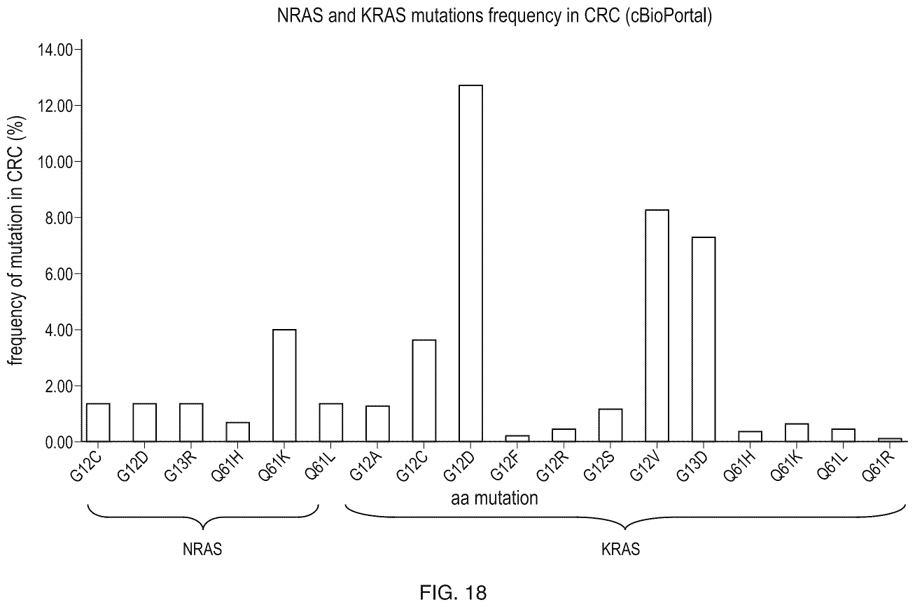

As shown in FIG. 18, NRAS is also mutated in colorectal cancer, but at a lower frequency than KRAS, based on analysis of data available in cBioPortal.

In this example, animals are administered an immunomodulatory therapeutic composition that includes an mRNA encoding at least one activating oncogene mutation peptide, e.g., at least one activating KRAS mutation, alone or in combination with an immune potentiator mRNA construct, e.g. a constitutively active STING mRNA construct, e.g., encoding a sequence as shown in any of SEQ ID NOs: 1-10, such as for example a mRNA construct encoding a constitutively active human STING protein comprising a V155M mutation, having the amino acid sequence shown in SEQ ID NO: 1 and encoded the nucleotide sequence shown in SEQ ID NO: 199.

Exemplary KRAS mutant peptide sequences and mRNA constructs are shown in Tables 7-9.

TABLE 7
KRAS mutant peptide sequences

| | 9 AA | | |
|---|---|---|---|
| | sequence | 15mer | 25mer |
| G12D | VVGADGVGK | MKLVVVGADGV | MTEYKLVVVGADGVGK |
| | (SEQ ID NO: | GKSAL | SALTIQLIQ |
| | 180) | (SEQ ID NO: | (SEQ ID NO: 98) |
| | | 95) | |
| G12V | VVGAVGVGK | MKLVVVGAVGV | MTEYKLVVVGAVGVGK |
| | (SEQ ID NO: | GKSAL | SALTIQLIQ |
| | 181) | (SEQ ID NO: | (SEQ ID NO: 99) |
| | | 96) | |
| G13D | VGAGDVGKS | MLVVVGAGDVG | MTEYKLVVVGAGDVGK |
| | (SEQ ID NO: | KSALT | SALTIQLIQ |
| | 182) | (SEQ ID NO: | (SEQ ID NO: 100) |
| | | 97) | |
| G12C | VVGACGVGK | MKLVVVGACGV | MTEYKLVVVGACGVGK |
| | (SEQ ID NO: | GKSA | SALTIQLIQ |
| | 183) | (SEQ ID NO: | (SEQ ID NO: 131) |
| | | 184) | |
| WT | | | MTEYKLVVVGAGGVGK |
| | | | SALTIQLIQ |
| | | | (SEQ ID NO: 133) |

TABLE 8
KRAS mutant amino acid sequences

| KRAS MUTANT | AMINO ACID SEQUENCE |
|---|---|
| KRAS(G12D)15mer | MKLVVVGADGVGKSAL (SEQ ID NO: |
| | 95) |
| KRAS(G12V)15mer | MKLVVVGAVGVGKSAL (SEQ ID NO: |
| | 96) |
| KRAS(G13D)15mer | MLVVVGAGDVGKSALT (SEQ ID NO: |
| | 97) |
| KRAS(G12D)25mer | MTEYKLVVVGADGVGKSALTIQLIQ |
| | (SEQ ID NO: 98) |
| KRAS(G12V)25mer | MTEYKLVVVGAVGVGKSALTIQLIQ |
| | (SEQ ID NO: 99) |
| KRAS(G13D)25mer | MTEYKLVVVGAGDVGKSALTIQLIQ |
| | (SEQ ID NO: 100) |
| KRAS(G12D)15mer\hat{ }3 | MKLVVVGADGVGKSALKLVVVGADGVGKSA |
| | LKLVVVGADGVGKSAL (SEQ ID NO: |
| | 101) |
| KRAS(G12V)15mer\hat{ }3 | MKLVVVGAVGVGKSALKLVVVGAVGVGKSA |
| | LKLVVVGAVGVGKSAL (SEQ ID NO: |
| | 102) |
| KRAS(G13D)15mer\hat{ }3 | MLVVVGAGDVGKSALTLVVVGAGDVGKSAL |
| | TLVVVGAGDVGKSALT (SEQ ID NO: |
| | 103) |
| KRAS(G12D)25mer\hat{ }3 | MTEYKLVVVGADGVGKSALTIQLIQMTEYK |
| | LVVVGADGVGKSALTIQLIQMTEYKLVVVG |
| | ADGVGKSALTIQLIQ (SEQ ID NO: |
| | 104) |
| KRAS(G12V)25mer\hat{ }3 | MTEYKLVVVGAVGVGKSALTIQLIQMTEYK |
| | LVVVGAVGVGKSALTIQLIQMTEYKLVVVG |
| | AVGVGKSALTIQLIQ (SEQ ID NO: |
| | 105) |
| KRAS(G13D)25mer\hat{ }3 | MTEYKLVVVGAGDVGKSALTIQLIQMTEYK |
| | LVVVGAGDVGKSALTIQLIQMTEYKLVVVG |
| | AGDVGKSALTIQLIQ (SEQ ID NO: |
| | 106) |
| KRAS(G12C)25mer | MTEYKLVVVGACGVGKSALTIQLIQ (SEQ |
| | ID NO: 131) |
| KRAS(G12C)25mer\hat{ }3 | MTEYKLVVVGACGVGKSALTIQLIQMTEYK |
| | LVVVGACGVGKSALTIQLIQMTEYKLVVVG |
| | ACGVGKSALTIQLIQ (SEQ ID NO: |
| | 132) |
| KRAS(WT)25mer | MTEYKLVVVGAGGVGKSALTIQLIQ (SEQ |
| | ID NO: 133) |

TABLE 9
KRAS mutant antigen mRNA sequences

| mRNA | Orf Sequence | |
|---|---|---|
| Name | (Amino Acid) | Orf Sequence (Nucleotide) |
| KRAS | MTEYKLVVVGADG | ATGACCGAGTACAAGCTGGTGGTGG |
| (G12D) | VGKSALTIQLIQ | TGGGCGCCGACGGCGTGGGCAAGAG |
| 25mer | (SEQ ID NO: 98) | CGCCCTGACCATCCAGCTGATCCAG |
| | | (SEQ ID NO: 185) |
| KRAS | MTEYKLVVVGAVG | ATGACCGAGTACAAGCTGGTGGTGG |
| (G12V) | VGKSALTIQLIQ | TGGGCGCCGTGGGCGTGGGCAAGAG |
| 25mer | (SEQ ID NO: 99) | CGCCCTGACCATCCAGCTGATCCAG |
| | | (SEQ ID NO: 186) |
| KRAS | MTEYKLVVVGAGD | ATGACCGAGTACAAGCTGGTGGTGG |
| (G13D) | VGKSALTIQLIQ | TGGGCGCCGGCGACGTGGGCAAGAG |
| 25mer | (SEQ ID NO: | CGCCCTGACCATCCAGCTGATCCAG |
| | 100) | (SEQ ID NO: 187) |
| KRAS | MTEYKLVVVGADG | ATGACCGAGTACAAGTTAGTGGTTG |
| (G12D) | VGKSALTIQLIQMTE | TGGGCGCCGACGGCGTGGGCAAGAG |
| 25mer\hat{ }3 | YKLVVVGADGVGK | CGCCCTCACCATCCAGCTTATCCAG |
| | SALTIQLIQMTEYKL | ATGACGGAATATAAGTTAGTAGTAG |
| | VVVGADGVGKSAL | TGGGAGCCGACGGTGTCGGCAAGTC |
| | TIQLIQ (SEQ ID | CGCTTTGACCATTCAACTTATTCAG |
| | NO: 104) | ATGACAGAGTATAAGCTGGTCGTTG |
| | | TAGGCGCAGACGGCGTTGGAAAGTC |
| | | GGCACTGACGATCCAGTTGATCCAG |
| | | (SEQ ID NO: 188) |
| KRAS | MTEYKLVVVGAVG | ATGACCGAGTACAAGCTCGTCGTGG |
| (G12V) | VGKSALTIQLIQMTE | TGGGCGCCGTGGGCGTGGGCAAGAG |
| 25mer\hat{ }3 | YKLVVVGAVGVGK | CGCCCTAACCATCCAGTTGATCCAG |
| | SALTIQLIQMTEYKL | ATGACCGAATATAAGCTCGTGGTAG |
| | VVVGAVGVGKSAL | TCGGAGCGGTGGGCGTTGGCAAGTC |
| | TIQLIQ (SEQ ID | AGCGCTAACAATACAACTAATCCAA |
| | NO: 105) | ATGACCGAATACAAGCTAGTTGTAG |
| | | TCGGTGCCGTCGGCGTTGGAAAGTC |
| | | AGCCCTTACAATTCAGCTCATTCAG |
| | | (SEQ ID NO: 189) |
| KRAS | MTEYKLVVVGAGD | ATGACCGAGTACAAGCTCGTAGTGG |
| (G13D) | VGKSALTIQLIQMTE | TTGGCGCCGGCGACGTGGGCAAGAG |
| 25mer\hat{ }3 | YKLVVVGAGDVGK | CGCCCTAACCATCCAGCTCATCCAG |
| | SALTIQLIQMTEYKL | ATGACAGAATATAAGCTTGTGGTTG |
| | VVVGAGDVGKSAL | TGGGAGCAGGAGACGTGGGAAAGAG |
| | TIQLIQ (SEQ ID | TGCGTTGACGATTCAACTCATACAG |
| | NO: 106) | ATGACCGAATACAAGTTGGTGGTGG |
| | | TCGGCGCAGGTGACGTTGGTAAGTC |
| | | TGCACTAACTATACAACTGATCCAG |
| | | (SEQ ID NO: 190) |
| KRAS | MTEYKLVVVGACG | ATGACCGAGTACAAGCTGGTGGTGG |
| (G12C) | VGKSALTIQLIQ | TGGGCGCCTGCGGCGTGGGCAAGAG |
| 25mer | (SEQ ID NO: | TCGCCCTGACCATCCAGCGATCCAG |
| | 131) | (SEQ ID NO: 191) |
| KRAS | MTEYKLVVVGACG | ATGACCGAGTACAAGCTCGTGGTTG |
| (G12C) | VGKSALTIQLIQMTE | TTGGCGCCTGCGGCGTGGGCAAGAG |
| 25mer\hat{ }3 | YKLVVVGACGVGK | CGCCCTCACCATCCAGCTCATCCAG |
| | SALTIQLIQMTEYKL | ATGACAGAGTATAAGTTAGTCGTTG |
| | VVVGACGVGKSAL | TCGGAGCTTGCGGAGTTGGAAAGTC |
| | TIQLIQ (SEQ ID | GGCGCTCACCATTCAACTCATACAA |
| | NO: 132) | ATGACAGAATATAAGTTAGTGGTGG |
| | | TGGGTGCGTGTGGCGTTGGCAAGAG |
| | | TGCGCTTACTATCCAGCTCATTCAG |
| | | (SEQ ID NO: 192) |
| KRAS | MTEYKLVVVGAGG | ATGACCGAGTACAAGCTGGTGGTGG |
| (WT) | VGKSALTIQLIQ | TGGGCGCCGGCGGCGTGGGCAAGAG |
| 25mer | (SEQ ID NO: | CGCCCTGACCATCCAGCTGATCCAG |
| | 133) | (SEQ ID NO: 193) |

Chemistry: uridines modified N1-methyl
pseudouridine (m1Ψ)
Cap: C1
Tail: T100
5′ UTR Sequence (standard 5′ Flank
(includes Production FP + T7 site + 5′UTR)):
(SEQ ID NO: 21)
TCAAGCTTTTGGACCCTCGTACAGAAGCTAATACGACTCACTATAG
GGAAATAAGAGAGAAAAGAAGAGTAAGAAGAAATATAAGAGCCACC
5′ UTR Sequence (No Promoter):
(SEQ ID NO: 194)
GGGAAATAAGAGAGAAAAGAAGAGTAAGAAGAAATATAAGAGCCAC
C
3′ UTR Sequence (Human 3′ UTR no XbaI):
(SEQ ID NO: 22)
TGATAATAGGCTGGAGCCTCGGTGGCCATGCTTCTTGCCCCTTGGG
CCTCCCCCCAGCCCCTCCTCCCCTTCCTGCACCCGTACCCCCGTGG
TCTTTGAATAAAGTCTGAGTGGGCGGC

In a first study to examine the effect of a STING immune potentiator mRNA construct on KRAS antigen responses in vivo, HLA-A*2:01 Tg mice (Taconic, strain 9659F, n=4) are administered mRNA encoding mutated KRAS as follows: mRNA encoding mutated KRAS (alone or in combination with STING) administered on day 1, bleed taken on day 8, mRNA encoding mutated KRAS (alone or in combination with STING) administered on day 15, animal sacrificed on day 22. The test groups are shown in Table 10 as follows:

TABLE 10

| | | Test/Control | Immune | | | Dosing |
|---|---|---|---|---|---|---|
| TEST group | Group | Material | Potentiator | Vehicle | Route | Regimen |
| KRAS-MUT | 1 | KRAS G12D | None (NTFIX) | Compound 25 | IM | Day 1, 15 |
| | 2 | KRAS G12V | None (NTFIX) | Compound 25 | IM | Day 1, 15 |
| | 3 | KRAS G13D | None (NTFIX) | Compound 25 | IM | Day 1, 15 |
| KRAS-MUT + | 4 | KRAS G12D | STING V155M) | Compound 25 | IM | Day 1, 15 |
| STING | 5 | KRAS G12V | STING V155M) | Compound 25 | IM | Day 1, 15 |
| | 6 | KRAS G13D | STING V155M) | Compound 25 | IM | Day 1, 15 |
| No Ag | 7 | NTFIX | NTFIX | Compound 25 | IM | Day 1, 15 |
| STING Only | 8 | NTFIX | STING V155M) | Compound 25 | IM | Day 1, 15 |

mRNA is administered to animals at a dose of 0.5 mg/kg (10 μg per 20-g animal). The KRAS and STING constructs are administered at a 1:1 ratio. Ex vivo restimulation (1 μg/ml per peptide) is tested for 4 hours at 37 degrees Celsius in the presence of GolgiPlug (Brefeldin A). Intracellular cytokine staining (ICS) is tested for KRAS G12D, KRAS G12V, KRAS G13D, KRAS G12WT, KRAS G13WT, and no peptide.

mRNA encoding KRAS mutations, alone or in combination with mRNA encoding constitutively active STING, is tested for the ability to generate T cells. Efficacy of mRNA encoding KRAS mutations is compared, for example, to peptide vaccination. The effect of the STING immune potentiator is determined by comparing treatment with the KRAS mutant peptides alone versus in combination with the STING immune potentiator. For example, CD8 vaccine responses can be assessed by intracellular staining (ICS) for IFN-γ and/or TNF-α as described herein. Enhanced ICS responses for IFN-γ and/or TNF-α in mice treated with the KRAS mutant peptide vaccine in combination with the STING immune potentiator mRNA construct, as compared to treatment with the KRAS mutant peptide vaccine mRNA construct alone, indicates that the STING immune potentiator enhances KRAS-specific CD8 vaccine responses.

In a second study to examine the effect of the STING immune potentiator mRNA construct on immune responses to various different forms of the mutant KRAS peptide antigen mRNA constructs, HLA*A*11:01 Tg mice (Taconic, strain 9660F, n=4) are administered mRNA encoding various different forms of mutated KRAS peptide antigen mRNA constructs in combination with a STING immune potentiator mRNA construct as follows: mRNA encoding mutated KRAS in combination with STING administered on day 1, bleed taken on day 8, mRNA encoding mutated KRAS in combination with STING administered on day 15, animal sacrificed on day 22.

The types of mutated KRAS constructs tested were as follows: (i) mRNA encoding a single mutant KRAS 25mer peptide antigen containing either the G12D, G12V, G13D or G12C mutation (“singlet”); (ii) mRNA encoding a concatemer of three 25mer peptide antigens (thus creating a 75mer), one of each containing the G12D, G12V and G13D mutations (“KRAS-3MUT”); (iii) mRNA encoding a concatemer of four 25mer peptide antigens (thus creating a 100mer), one of each containing the G12D, G12V, G13D and G12C mutations (“KRAS-4MUT”); or (iv) four separate mRNAs coadministered together, each encoding a single mutant KRAS 25mer peptide antigen containing either the G12D, G12V, G13D or G12C mutation (“Single x 4”).

The amino acid and nucleotide sequences of the G12D 25mer are shown in SEQ ID NOs: 98 and 185, respectively. The amino acid and nucleotide sequences of the G12V 25mer are shown in SEQ ID NOs: 99 and 186, respectively. The amino acid and nucleotide sequences of the G13D 25mer are shown in SEQ ID NOs: 100 and 187, respectively. The amino acid and nucleotide sequences of the G12C 25mer are shown in SEQ ID NOs: 131 and 191 respectively. The amino acid and nucleotide sequences of the KRAS-3MUT 75mer are shown in SEQ ID NOs: 195 and 196, respectively. The amino acid and nucleotide sequences of the KRAS-4MUT 100mer are shown in SEQ ID NOs: 197 and 198, respectively. Additional nucleotide sequences of KRAS-4MUT 100mer are shown in SEQ ID NOs: 1321 and 1322.

The test groups are shown in Table 11 as follows:

TABLE 11

| | | Test/Control | Immune | | | Dosing |
|---|---|---|---|---|---|---|
| TEST group | Group | Material | Potentiator | Vehicle | Route | Regimen |
| KRAS-MUT | 1 | KRAS G12D | STING (V155M) | Compound 25 | IM | Day 1, 15 |
| Singlet | 2 | KRAS G12V | STING (V155M) | Compound 25 | IM | Day 1, 15 |
| | 3 | KRAS G13D | STING (V155M) | Compound 25 | IM | Day 1, 15 |
| | 4 | KRAS G12C | STING (V155M) | Compound 25 | IM | Day 1, 15 |
| KRAS-MUT | 5 | KRAS-3MUT | STING (V155M) | Compound 25 | IM | Day 1, 15 |
| Concatemer | 6 | KRAS-4MUT | STING (V155M) | Compound 25 | IM | Day 1, 15 |
| Single X 4 | 7 | G12D + G12V + | STING (V155M) | Compound 25 | IM | Day 1, 15 |
| | | G12C + G13D | | | | |
| STING Only | 8 | NTFIX | STING (V155M) | Compound 25 | IM | Day 1, 15 |

mRNA is administered to animals at a dose of 0.5 mg/kg (10 μg per 20-g animal). The KRAS and STING constructs are administered at a 1:1 ratio. Ex vivo restimulation (lug/ml per peptide) is tested for 4 hours at 37 degrees Celsius in the presence of GolgiPlug (Brefeldin A). Intracellular cytokine staining (ICS) is tested for KRAS G12D, KRAS G12V, KRAS G13D, G12C, KRAS G12WT, KRAS G13WT, and no peptide.

The ability of the various mRNAs encoding KRAS mutations in combination with mRNA encoding constitutively active STING to generate T cell responses is tested to allow for comparison of the effect of the STING immune potentiator on the various different KRAS constructs. For example, CD8 vaccine responses can be assessed by intracellular staining (ICS) for IFN-γ and/or TNF-α as described herein.

### Example 10: Prophylactic or Therapeutic Vaccination with HPV Vaccine in Combination with STING Immune Potentiator Inhibits Tumor Growth

In this example, mice were treated with an HPV vaccine in combination with a STING immune potentiator either prior to, at the same time as, or after challenge with TC1 tumor cells. TC-1 is an HPV16 E7-expressing murine tumor model known in the art (see e.g., Bartkowiak et al. (2015) Proc. Natl. Acad. Sci. USA 112: E5290-5299). The HPV vaccines used in this example were mRNA constructs encoding either intracellular or soluble forms of HPV 16 antigens E6 and E7, referred to herein as iE6/E7 and sE6/E7, respectively, as described in Example 5. The constitutively active STING immune potentiator used in this example contained a V155M mutation, as described in Example 5. The HPV vaccine construct and the immune potentiator construct were coformulated in MC3 lipid nanoparticles. Certain mice were also treated with an immune checkpoint inhibitor (either anti-CTLA-4 or anti-PD-1).

In a first set of experiments examining the prophylactic activity of the HPV+STING vaccination, C57/B6 mice were treated by intramuscular injection with 0.5 mg/kg of the HPV+STING vaccine (encoding either sE6/E7 or iE6/E7) on either (i) days-7 and -14, or (ii) days 1 and 8, followed by subcutaneous injection of 2×10⁵ TC1 cells on day 1. Certain mice were also treated on days 6, 9 and 12 with either anti-CTLA-4 (clone 9H10) or anti-PD-1 (RMP1-14). Representative results, reported as tumor volume over time, are shown in the graphs of FIG. 19A-19C, wherein FIGS. 19A and 19B show data for mice treated on days-14 and -7 with either sE6/E7 (FIG. 19A) or iE6/E7 (FIG. 19B) and FIG. 19C shows data for mice treated on days 1 and 8 with sE6/E7. The results demonstrate that all of the mice treated with the HPV+STING vaccine (alone or in combination with immune checkpoint inhibitors) showed complete inhibition of tumor growth over several weeks, as compared to the control mice (treated with the control mRNA construct NTFIX, alone or in combination with an immune checkpoint inhibitor). Thus, these experiments demonstrate that prophylactic vaccination (i.e., prior to or at the same time as tumor challenge) with the HPV vaccine together with the STING immune potentiator is effective in preventing growth of HPV-expressing tumor cells in vivo.

In a second set of experiments examining the therapeutic activity of the HPV +STING vaccination, C57/B6 mice were administered 2×10⁵ TC1 cells subcutaneously on day 1, followed by treatment by intramuscular injection with 0.5 mg/kg of the HPV+STING vaccine (encoding sE6/E7) on days 8 and 15. Certain mice were also treated on days 13, 16 and 19 with either anti-CTLA-4 (clone 9H10) or anti-PD-1 (RMP1-14). Representative results, reported as tumor volume over time, are shown in the graphs of FIGS. 20A-20I. The results demonstrate that the mice treated with the HPV+STING vaccine (alone or in combination with immune checkpoint inhibitors) showed tumor regression (FIGS. 20A-20C), as compared to the control mice treated with the control mRNA construct NTFIX, alone or in combination with an immune checkpoint inhibitor (FIGS. 20D-20F) or the control mice treated with the sE6/E7 construct in combination with the control DMXAA compound (a chemical activator of STING), alone or in combination with an immune checkpoint inhibitor (FIGS. 20G-20I). Thus, these experiments demonstrate that therapeutic vaccination (i.e., subsequent to tumor challenge) with the HPV vaccine together with the STING immune potentiator is effective in inducing regression of HPV-expressing tumors in vivo.

In a third series of experiments, to examine the efficacy of the HPV-STING therapeutic vaccine in larger TC1 tumors, C₅₇/B6 mice were administered 2×105 TC1 cells subcutaneously and tumors were allowed to grow to a volume of either 200 mm³ or 300 mm³, which was then designated as day 1. Mice were then treated on days 1 and 8 by intramuscular injection with the HPV+STING vaccine (encoding sE6/E7). The treatment groups and corresponding dosages are provided in Table 12.

TABLE 12

| Tumor size | | Vax dose 1 | Vax dose 2 |
|---|---|---|---|
| (mm³) | Group | (day 1), μg | (day 8) μg |
| 200 | PBS | none | none |
| | sE6/7 + NFTIX (1:1) | 10 | 10 |
| | sE6/7 + STING (1:1) | 10 | 5 |
| | sE6/7 + NFTIX (1:1) + | 10 | 10 |
| | 200 μg DMXAA | | |
| 300 | PBS | none | none |
| | sE6/7 + NFTIX (1:1) | 10 | 10 |
| | sE6/7 + STING (1:1) | 10 | None |
| | sE6/7 + NFTIX (1:1) + | 10 | 10 |
| | 200 μg DMXAA | | |

The results are shown in FIG. 21, which show tumor volume over the course of 22 days (upper graphs are for 200 mm³ tumors and lower graphs are for 300 mm³ tumors). The results demonstrate that the HPV-STING therapeutic vaccine exhibits efficacy in inhibiting larger HPV-expressing tumors in vivo.

### Example 11: Determining Optimal Antigen: Immune Potentiator Mass Ratio in mRNA Vaccine Design

In this example, studies were performed in animals treated with an antigen of interest (Ag) in combination with an immune potentiator at different Ag: Immune Potentiator ratios, followed by examination of T cell responses to the antigen, to determine optimal Ag: Immune Potentiator ratios in enhancing the immune response to the antigen of interest.

In a first set of experiments, mice were treated with an MC38 vaccine encoding an ADR concatemer of three 25mer mutant peptides containing tumor neoepitopes derived from Adpgk, Dpagt1, and Reps1 (this vaccine is also referred to herein as ADRvax), as described in Example 6, in combination with a constitutively active STING immune potentiator construct. The constitutively active STING immune potentiator used in this example contained a V155M mutation, as described in Example 5. The ADRvax and STING constructs were coformulated in an SM102 cationic lipid nanoparticle (comprising Compound 25) at varying Ag:STING ratios, according to the study design summarized below in Table 13.

TABLE 13

| | | | STING | | Total | | | |
|---|---|---|---|---|---|---|---|---|
| | Ag:STING | Ag dose | dose | NTFIX | mRNA | | | Dosing |
| Group | ratio | (μg) | (μg) | (μg) | (μg) | Vehicle | Route | Regimen |
| 1 | No Ag | 0 | 3 | 3 | 6 | SM102 | IM | Day 1, 15 |
| | control | | | | | | | |
| 2 | 1:1 | 3 | 3 | 0 | | | | |
| 3 | 5:1 | | 0.6 | 2.4 | | | | |
| 4 | 10:1 | | 0.3 | 2.7 | | | | |
| 5 | 20:1 | | 0.15 | 2.85 | | | | |
| 6 | 1:0 (No | | 0 | 3 | | | | |
| | STING) | | | | | | | |
| 7 | 1:1 | 5 | 5 | 0 | 10 | | | |
| 8 | 1:0 (No | | 0 | 5 | | | | |
| | STING) | | | | | | | |

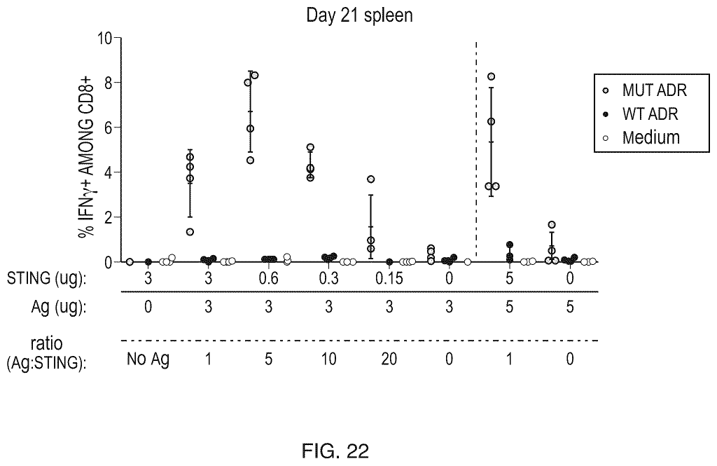

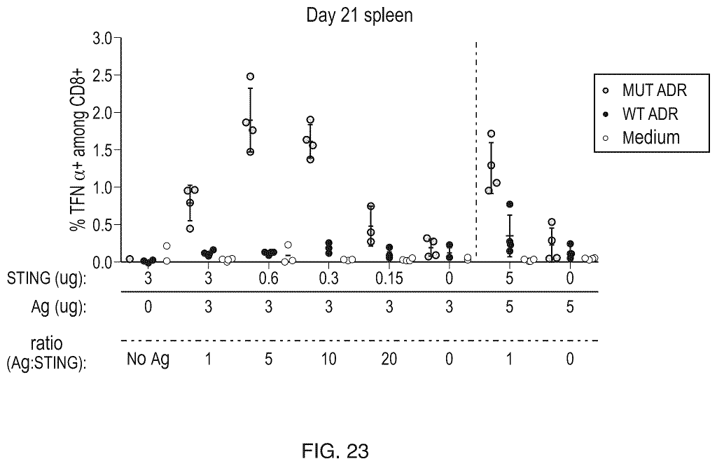

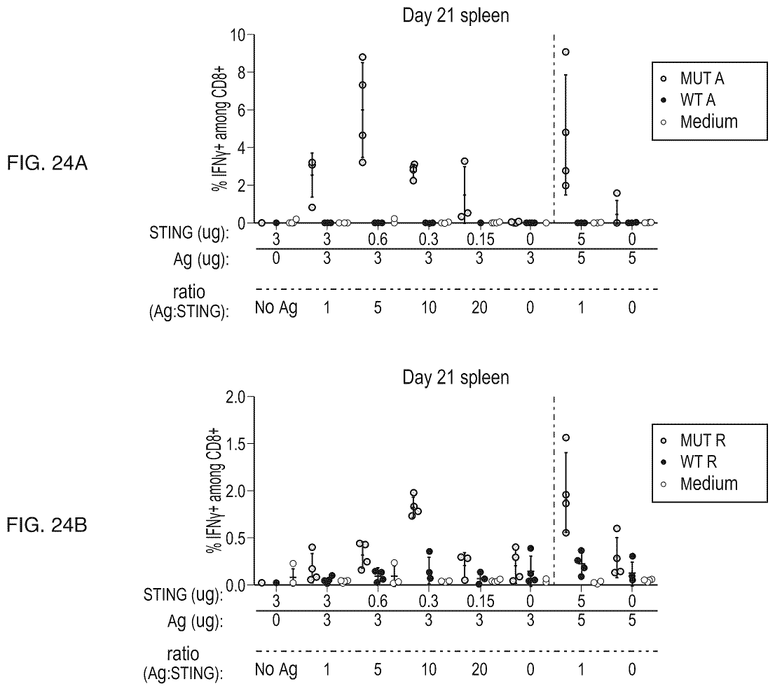

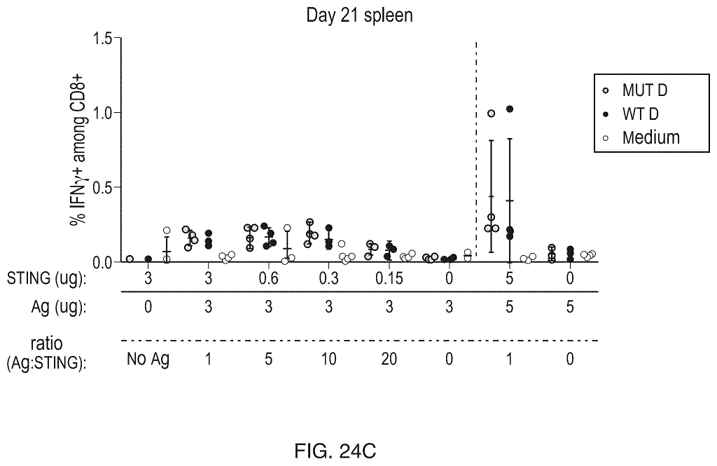

Mice were dosed intramuscularly on days 1 and 15. At day 21, CD8⁺ spleen cells from mice in each test group were restimulated ex vivo for 4 hours at 37 degrees C. in the presence of GolgiPlug™ (containing Brefeldin A; BD Biosciences) with either wild-type or mutant MC38 ADR peptides (1 μg/ml per peptide, pooled) and CD8 vaccine responses were assessed by intracellular staining (ICS) for IFN-γ or TNF-α. Representative ICS results for MC38 ADR-specific responses by day 21 CD8⁺ spleen cells for IFN-γ are shown in FIG. 22 and for TNF-α are shown in FIG. 23. Comparable results were observed with day 21 PBMCs. Furthermore, the experiment was carried out through 54 days, with the day 54 spleen cells results being comparable to the results observed for the day 21 spleen cells. Additionally, CD8 vaccine responses to each of the three individual epitopes within ADRvax (i.e., peptides Adpk1, Reps1 and Dpagt1) were also assessed by ICS for IFN-γ following stimulation with the individual epitopes. The results are shown in FIG. 24A (for peptide Adpk1), FIG. 24B (for peptide Reps1) and FIG. 24C (for peptide Dpagt1).

The results demonstrate that all Ag:STING ratios tested (ranging from 1:1 to 20:1) showed an adjuvant effect of STING as compared to control. For the ADRvax antigen as a whole, the optimal Ag:STING ratio was found to be 5:1. For the individual peptide epitopes within ADRvax, the optimal Ag:STING ratio for the Adpgk1 peptide was 5:1, whereas the optimal Ag:STING ratio for the Reps1 peptide was 10:1 (the responses to the third peptide, Dpagt1, were very low with or without STING, consistent with it being a non-dominant epitope as was known in the art). STING was also found to increase the total percentage of CD8+ cells among CD45+ T cells, with dose responses observed (data not shown) and was found to increase the total percentage of CD62L cells among CD44hi CD8+ cells (effector/memory subset), with dose responses observed (data not shown). Furthermore, results obtained from PBMC cells were consistent with the spleen cell results (data not shown). Thus, these experiments confirmed the ability of STING to act as an immune potentiator in enhancing immune responses against the ADRvax antigen and, moreover, demonstrated the determination of an optimal Ag: Immune Potentiator ratio for treatment, with ratios other than 1:1 being found to be most optimal (e.g., ratios of 5:1 or 10:1 being more effective than 1:1). The results further indicate that the optimal Ag: Immune Potentiator ratio may differ depending on the particular antigen of interest used.

In a second set of experiments, non-human primates were treated with an HPV vaccine encoding intracellular E6/E7 (iE6/E7), as described in Example 5, in combination with the constitutively active STING immune potentiator construct at varying Ag:STING ratios (coformulated in SM102 cationic lipid nanoparticles), according to the study design summarized below in Table 14.

TABLE 14

| | | Ag:STING | μg | μg | μg | | Total Ag |
|---|---|---|---|---|---|---|---|
| Group | Treatment | Ratio | Ag | STING | NTFIX | n | Dose |
| 1 | STING only | — | — | 100 | — | 3 | 100 μg |
| 2 | Ag:STING | 1:1 | 50 | 50 | — | | |
| 3 | Ag:STING | 5:1 | 83.33 | 16.67 | — | | |
| 4 | Ag:STING | 10:1 | 90.9 | 9.09 | | | |
| 5 | Ag only | — | 90 | — | 10 | | |

No clinical findings were observed 24 hours after the first dose (administered intramuscularly), indicating no injection site reactions and that the initial treatment was received safely. After an initial dosing on day 1, animals have a two week recovery period and then are given a second dose at day 14, followed by another two week recovery period. Further safety analysis is determined by clinical pathology (clinical chemistry, hematology and coagulation) at days 2, 16 and 30. Anti-antibody and ELISpot analysis or ICS for IFN-γ for CD4 and CD8 cells are performed to assess enhancement of immune responses to the HPV vaccine by STING at the varying ratios tested.

In a third set of experiments, a model concatemeric antigen using known murine epitopes was tested in mice in combination with the constitutively active STING immune potentiator at varying ratios. The concatemeric antigen, referred to herein as CA-132, comprises 20 known murine epitopes thought to be presented on MHC Class I and Class II antigens of the CB6 mouse. These epitopes were sourced from the IEDB.org website, a public database of epitopes sourced from the literature. Class I epitopes are expected to be presented on MHC Class I molecules and trigger a CD8+ response, while Class II epitopes are expected to be presented on MHC Class II molecules and trigger CD4+ T cell responses. The CA-132 antigen construct encodes both Class I and Class II epitopes, allowing for assessment of both CD4 and CD8 T cell responses. Moreover, it is believed that inclusion of Class II epitopes in the concatemeric antigen (thus triggering a CD4 response) helps induce a stronger CD8 T cell response. Thus, the approach to the design of the CA-132 antigen can also be used in the design of other concatemeric antigen constructs (e.g., for personalized cancer vaccines or for bacterial vaccines, as described herein).

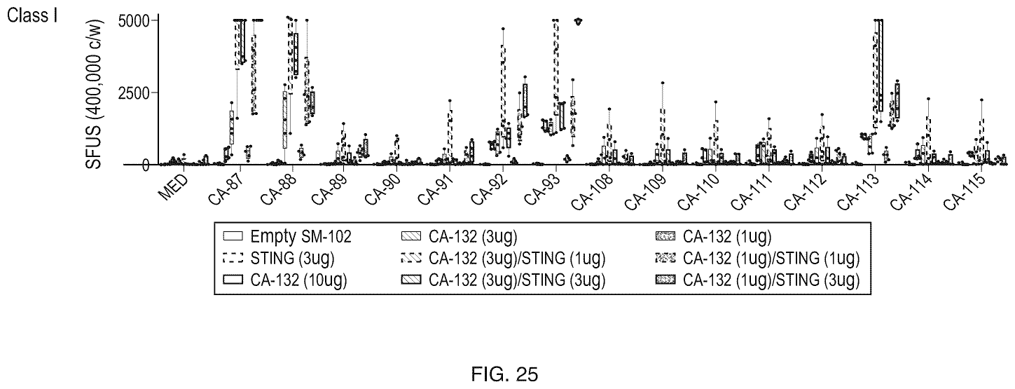

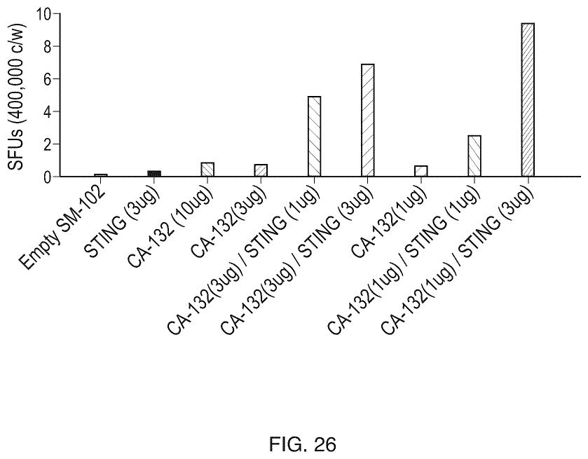

The CA-132 antigen construct and STING immune potentiator construct were coformulated in SM102 cationic lipid nanoparticles and administered intramuscularly to CB6 mice at the following dosages: CA-132 alone at 1 μg, 3 μg or 10 μg, STING alone at 3 μg, CA-132+STING at either 3 μg each or 1 μg each (1:1 ratio), CA-132 at 3 μg and STING at 1 μg (Ag:STING ratio of 3:1) or CA-132 at 1 μg and STING at 3 μg (Ag:STING ratio of 1:3). Antigen-specific T cell responses to the Class I epitopes within the CA-132 antigen construct were examined by ELISpot analysis for IFN-γ, the results of which are shown in FIG. 25. The results demonstrated an increase in IFN-γ responses to the Class I epitopes when formulated with STING. These results were confirmed by restimulation of CD8+ T cells with one of the Class I epitopes within the CA-132 antigen (epitope CA-87), followed by ELISpot analysis for IFN-γ, the results of which are shown in FIG. 26. These results demonstrated the ability of STING to immunopotentiate the antigen-specific CD8+ T cell responses at Ag:STING ratios of 1:1, 3:1 and 1:3.

In a fourth set of experiments, C₅₇/B16 mice were treated on days 1 and 14 with an HPV16 E7 vaccine (described in Example 5), in combination with the constitutively active STING immune potentiator construct at varying Ag:STING ratios. The mRNAs were coformulated in lipid nanoparticles comprising: Compound 25: Cholesterol: DSPC: PEG-DMG (at ratios of 50:38.5:10:1.5, respectively) according to the study design summarized below in Table 15.

TABLE 15

| | | Ag:STING | μg | μg |
|---|---|---|---|---|
| Group | Treatment | Ratio | Ag | STING |
| 1 | STING only | — | 0 | 3 |
| 2 | Ag:STING | 1:1 | 3 | 3 |
| 3 | Ag:STING | 5:1 | 3 | 0.6 |
| 4 | Ag:STING | 10:1 | 3 | 0.3 |
| 5 | Ag:STING | 20:1 | 3 | 0.15 |
| 6 | Ag only | 0 | 3 | 0 |
| 7 | Ag:STING | 1:1 | 5 | 5 |
| 8 | Ag only | 0 | 5 | 0 |

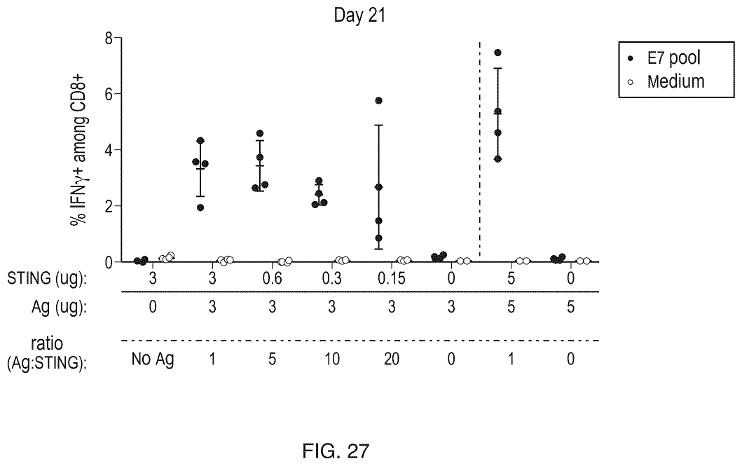

On day 21, mice were sacrificed and IFN-γ expression by CD8+ T cells was assessed by ICS as described herein. The results are shown in FIG. 27, which demonstrates a strong immunopotentiating effect of the STING mRNA construct at Ag:STING ratios of 1:1, 5:1 and 10:1.

In summary, these studies confirmed the ability of the STING immune potentiator construct to enhance immune responses to an antigen of interest and demonstrated the determination of optimal Ag:STING ratios for treatment.

### Example 12: Immune Potentiation by STING in Non-Human Primates

In this example, non-human primates (cynomolgus monkeys) were treated with mRNAs encoding an HPV vaccine in combination with a STING immune potentiator, followed by assessment of antigen-specific T cell and antibody responses. The HPV vaccine construct used in this example is described in Example 5. The constitutively active STING immune potentiator construct used in this example contained a V155M mutation, as described in Example 5. The HPV vaccine construct and the immune potentiator mRNA constructs were coformulated in lipid nanoparticles comprising: Compound 25: Cholesterol: DSPC: PEG-DMG, at ratios of 50:38.5:10:1.5, respectively. Different ratios of STING:Ag were tested. Control animals were treated with mRNAs encoding either the HPV antigens alone or the STING immune potentiator alone.

Fifteen male cynomolgus monkeys, 2-5 years old and weighing 2-5 kg, were treated according to the study design shown below in Table 16.

TABLE 16

| | | | Total mRNA | | STING | HPV Ag | |
|---|---|---|---|---|---|---|---|
| Group | Desc. | Ratio | (μg) | NTFIX | (μg) | (μg) | n |
| 1 | Ag only | | 100 | 10 | | 90 | 3 |
| 2 | STING only | | 100 | | 100 | 0 | 3 |
| 3 | STING:Ag | 1:1 | 100 | | 50 | 50 | 3 |
| 4 | STING:Ag | 1:5 | 100 | | 17 | 83 | 3 |
| 5 | STING:Ag | 1:10 | 100 | | 9 | 91 | 3 |

A pre-dose sample of PBMCs were collected on day −7, followed by treatment of the animals intramuscularly with the mRNA LNPs on day 1 and day 15. A post-dose sample of PBMCs was collected on day 29. No toxicity or other major clinical observations were noted during the study, indicating the mRNA LNPs were well-tolerated.

To examine the ability of the STING immune potentiator to enhance antigen-specific CD8+ T cell responses, intracellular cytokine staining (ICS) for TNFα and IL-2 was conducted. PBMCs were stimulated ex vivo with the HPV16 E6 peptide pool or the HPV16 E7 peptide pool for 6 hours at 37° C. Stimulation with PMA/ionomycin was used as a positive control and stimulation with medium alone was used as a negative control.

Representative results for ICS for TNFα are shown in FIGS. 28A-28C, wherein FIG. 28A shows results for ex vivo stimulation with the E6 peptide pool, FIG. 28B shows the results for ex vivo stimulation with the E7 peptide pool and FIG. 28C shows the results for ex vivo stimulation with the medium control. No increase in TNFα⁺ CD8 T cell frequency was observed between the pre- and post-dose group immunized with antigen alone (Group 1). Immunization with STING treatment alone (Group 2) had a marginal effect on TNFα+ CD8 T cell frequency. In contrast, groups immunized with STING+Ag (Groups 3, 4, 5) showed a significant increase in antigen-specific TNFα+ CD8 T cells. Furthermore, Group 5, which was immunized with a “matching” antigen dose of STING:Ag (1:10 ratio), showed a significant increase in antigen-specific TNFα+ CD8 T cells when compared to the Group 1 and Group 2 controls.

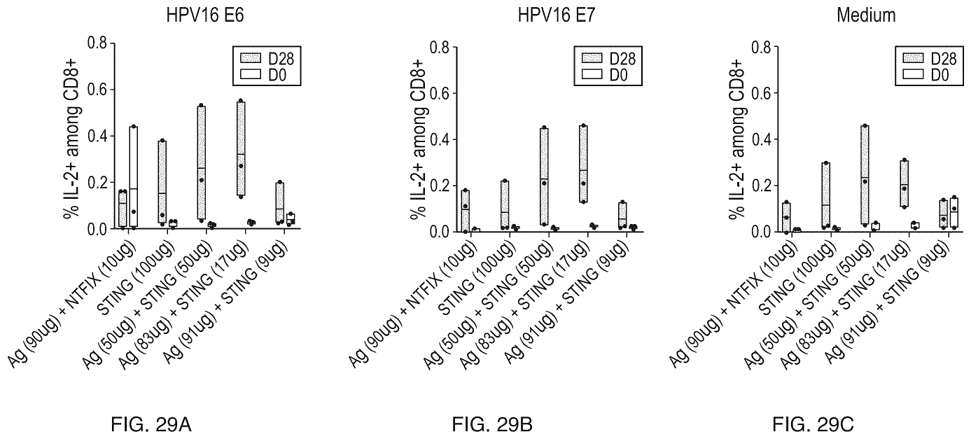

Representative results for ICS for IL-2 are shown in FIGS. 29A-29C, wherein FIG. 29A shows results for ex vivo stimulation with the E6 peptide pool, FIG. 29B shows the results for ex vivo stimulation with the E7 peptide pool and FIG. 29C shows the results for ex vivo stimulation with the medium control. A moderate increase in IL-2+ CD8 T cell frequency between the pre- and post-dose was observed in all immunized animals (Groups 1-5). However, the increase in IL-2+ CD8 T cells was most detectable in the groups treated with STING:Ag at ratios of 1:1 and 1:5 (Groups 3 and 4), whereas animals treated with STING:Ag at a 1:10 ratio did not exhibit increased IL-2+ CD8 T cells as compared to controls. The increase in IL-2 is consistent with the known ability of subsets of T cells to secrete IL-2 during active T cell responses.

To examine the effect of STING:Ag treatment in the NHPs on antigen-specific antibody responses, E6-specific and E7-specific ELISAs were performed. Plates were coated with either recombinant E6 (Prospec; #HPV-005 His HPV16 E6) or recombinant E7 (ProteinX; #2003207 His HPV16 E7). A mouse anti-E6 monoclonal antibody from Alpha Diagnostics International (#HPV16E6 1-M) was used as a positive control. A mouse anti-E7 monoclonal antibody from Fisher/Life Technologies (#280006-EA) was used as a positive control. An anti-mouse IgG-HRP antibody from Jackson ImmunoResearch (#715-035-150) was used as the secondary antibody for the positive controls. Anti-monkey IgG-HRP from Abcam (#ab112767) was used as the secondary antibody for the NHP serum.

Plates were coated with recombinant E6 or E7 (500 ng/well; 100 μl/well) at 4C overnight and then blocked with TBS SuperBlock for 1 hour at room temperature. Primary antibody was added (100 μl/well) and incubated for 1 hour at room temperature. Positive control antibodies were serially diluted. NHP serum was diluted 1:5000. After washing, secondary antibody was added (100 μl/well) and incubated for 1 hour at room temperature. Positive control anti-mouse IgG-HRP was diluted 1:5000. For the NHP serums, anti-monkey IgG-HRP was diluted 1:30,000. Color was developed for 5 minutes (anti-E6) or for 10 minutes (anti-E7), then stopped and read at 450 nm.

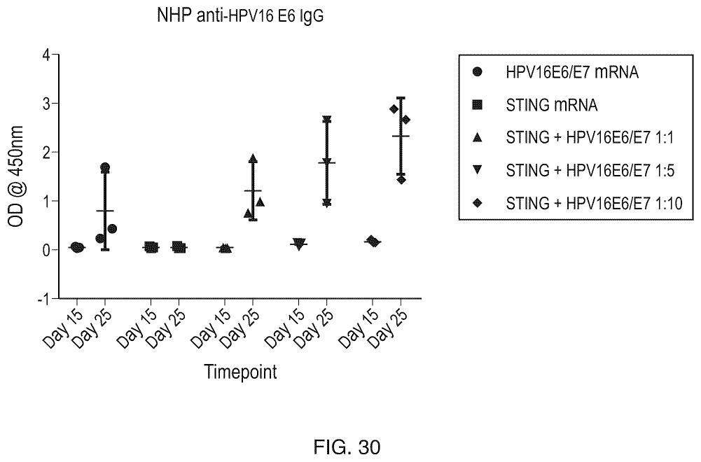

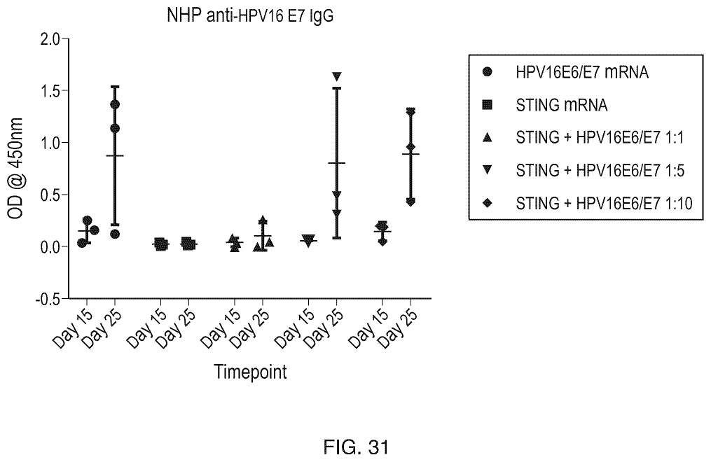

Representative results for anti-HPV16 E6 IgG are shown in FIG. 30. Representative results for anti-HPV16 E7 IgG are shown in FIG. 31. The results for both anti-E6 and anti-E7 demonstrate that treatment of the animals with STING:Ag, particularly at ratios of 1:5 and 1:10 led to increased antigen-specific antibody responses.

Accordingly, the results described herein for the non-human primate study confirm that STING immunopotentiates antigen-specific T cell and antibody responses against an mRNA vaccine antigen in vivo.

### Example 13: Immunogenicity of Various KRAS-STING Vaccines Formats in HLA*A11 Transgenic Mice

In this example, to examine the effect of the STING immune potentiator mRNA construct on immune responses to various different forms of the mutant KRAS peptide antigen mRNA constructs, HLA*A*11:01 Tg mice (Taconic, strain 9660F, n=3) were administered mRNA encoding various different forms of mutated KRAS peptide antigen mRNA constructs in combination with a STING immune potentiator mRNA construct as follows: mRNA encoding mutated KRAS in combination with STING administered on days 0 and 14, animals sacrificed on day 21. Mice were aged 6-9 weeks at day 0. mRNA was administered to the animals at a dose of 0.5 mg/kg (10 μg per 20-g animal). The KRAS and STING constructs are administered at a 5:1 ratio (Ag:STING). mRNA constructs were coformulated in an SM102 cationic lipid nanoparticle (comprising Compound 25).

The types of mutated KRAS constructs tested were as follows: (i) mRNA encoding a single mutant KRAS 25mer peptide antigen containing either the G12D, G12V, G13D or G12C mutation (“monomer”); (ii) mRNA encoding a concatemer of three 25mer peptide antigens (thus creating a 75mer), one of each containing the G12D, G12V and G13D mutations (“KRAS-3MUT concatemer”); (iii) mRNA encoding a concatemer of four 25mer peptide antigens (thus creating a 100mer), one of each containing the G12D, G12V, G13D and G12C mutations (“KRAS-4MUT concatemer”); or (iv) four separate mRNAs coadministered together, each encoding a single mutant KRAS 25mer peptide antigen containing either the G12D, G12V, G13D or G12C mutation (“pooled monomers”). The amino acid and nucleotide sequences of the constructs are as described in Example 9. An A11-viral epitope concatemer antigen was also tested in combination with STING or a control mRNA (NTFIX) (“validated A11 Ag”).

The test groups are shown in Table 17 as follows:

TABLE 17

| | | Test/Control | Immune | | | Dosing |
|---|---|---|---|---|---|---|
| TEST group | Group | Material | Potentiator | Vehicle | Route | Regimen |
| KRAS-MUT | 1 | KRAS G12D | STING (V155M) | Compound 25 | IM | Day 1, 14 |
| Monomer | 2 | KRAS G12V | STING (V155M) | Compound 25 | IM | Day 1, 14 |
| | 3 | KRAS G13D | STING (V155M) | Compound 25 | IM | Day 1, 14 |
| | 4 | KRAS G12C | STING (V155M) | Compound 25 | IM | Day 1, 14 |
| KRAS-MUT | 5 | KRAS-3MUT | STING (V155M) | Compound 25 | IM | Day 1, 14 |
| Concatemer | 6 | KRAS-4MUT | STING (V155M) | Compound 25 | IM | Day 1, 14 |
| | 7 | KRAS-4MUT.var1 | STING (V155M) | Compound 25 | IM | Day 1, 14 |
| Pooled | 8 | G12D + G12V + | STING (V155M) | Compound 25 | IM | Day 1, 14 |
| Monomers | | G12C + G13D | | | | |
| Validated | 9 | A11-Viral epitope | STING (V155M) | Compound 25 | IM | Day 1, 14 |
| A11 Ags | | concatemer | | | | |
| | 10 | A11-Viral epitope | NTFIX | Compound 25 | IM | Day 1, 14 |
| | | concatemer | | | | |

In a first set of experiments to evaluate antigen-specific CD8+ T cell responses to the KRAS antigens, day 21 spleen cells from the mice were restimulated ex vivo with KRAS monomer peptides (2 μg/ml per peptide) for 5 hours at 37 degrees Celsius in the presence of GolgiPlug (Brefeldin A). Intracellular cytokine staining (ICS) (IFN-γ) was performed for KRAS G12D (aa*7/8-16), KRAS G12V (aa*7/8-16), KRAS G13D (aa*7/8-16), G12C (aa*7/8-16), KRAS WT (aa*7/8-16) and no peptide.

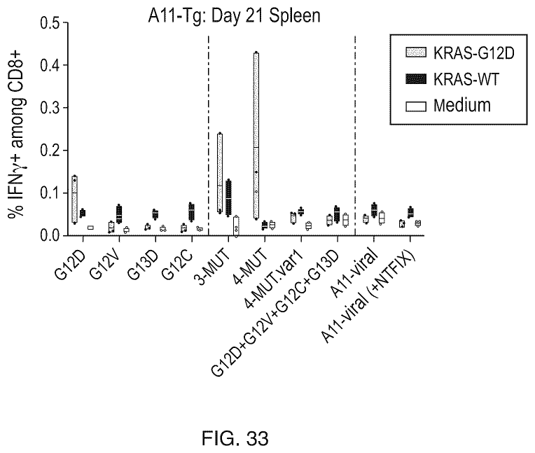

The ICS results for KRAS-G12V-specific responses are shown in FIG. 32. The ICS results for KRAS-G12D-specific responses are shown in FIG. 33. These results demonstrate that anti-KRAS-G12V and anti-KRAS-G12D specific CD8+ T cells were detected in mice immunized with the corresponding KRAS-STING vaccine (monomer or concatemer) and restimulated with the cognate peptide. Comparable % IFN-gamma positive CD8+ T cells were seen when the KRAS mutations were administered to the mice as a monomer or as concatemers. The responses observed with G12V were stronger than the responses observed with G12D. In this experiment, anti-KRAS G12C and anti-KRAS G13D responses were not observed (data not shown).

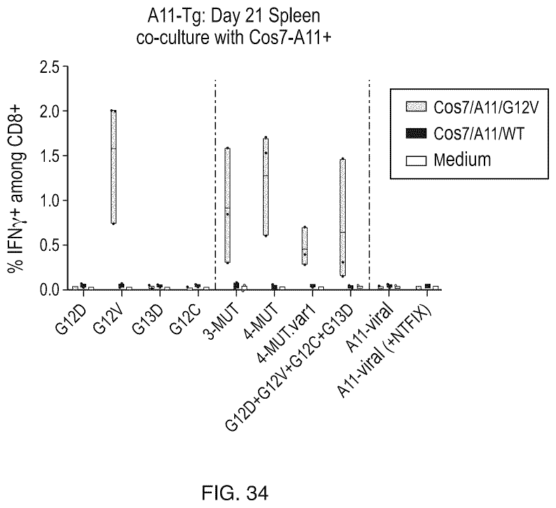

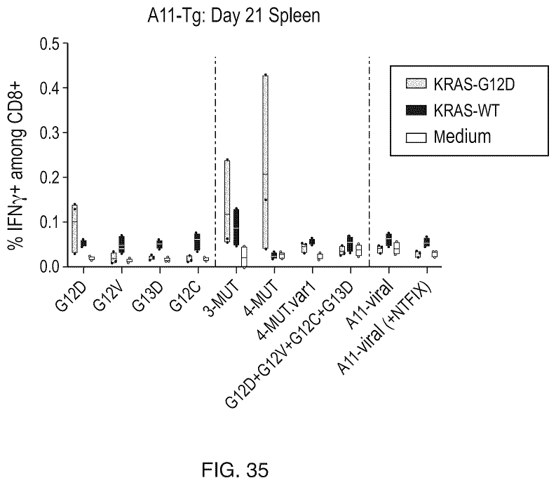

In a second set of experiments to evaluate antigen-specific CD8+ T cell responses to KRAS antigens, day 21 spleen cells from the mice were co-cultured with HLA*A11-expressing target cells (Cos7-A11 cells) that had been pulsed with the corresponding KRAS peptides (G12V, G12D or WT control), followed by ICS (IFN-γ). The Cos7-A11 co-culture results for KRAS-G12V-specific responses are shown in FIG. 34. The Cos7-A11 co-culture results for KRAS-G12D-specific responses are shown in FIG. 35. These results demonstrate that anti-KRAS-G12V and anti-KRAS-G12D specific CD8+ T cell responses were detected in mice immunized with the corresponding KRAS-STING vaccine (monomer or concatemer) and restimulated with the A11+ expressing cell line pulsed with G12V or G12D. Thus, the results in this second set of experiments with respect to detection of antigen-specific CD8+ T cell responses to the KRAS antigens were very similar to the results from the first set of experiments using restimulation with cognate peptides.

Finally, the ability of STING to potentiate antigen-specific response to known A*11-restricted viral epitopes was evaluated using day 21 spleen cells from the mice immunized with an A11-viral epitope concatemer. Eight viral epitopes (EBV BRLF1, FLU, HIV NEF, EBV, HBV core antigen, HCV, CMV and BCL-2L1) (25 amino acids each) were concatemerized and encoded by mRNA for use as an antigen in combination with STING in the A11-transgenic mice (treatment group 9 in Table 17). The A11-viral epitope concatemer was also co-administered with an NTFIX control mRNA (treatment group 10 in Table 17). Five of the eight epitopes (EBV BRLF1, FLU, HIV NEF, EBV, HBV core antigen) were validated A11 binders with relatively low predicted IC50s; the other three epitopes (HCV, CMV and BCL-2L1) had more moderate predicted affinities for A11 but have not been experimentally validated. The amino acid sequences for the viral epitopes, as well as their IC50s, are shown below in Table 18.

TABLE 18

| | | | | Liter- |
|---|---|---|---|---|
| | | | | ature |
| | | | | vali- |
| Gene | Peptide | ann_IC50 | % rank | dation |
| BV BRLF1 | ATIGTAMYK | 6.03 | 0.2 | Y |
| | (SEQ ID | | | |
| | NO: 1388) | | | |
| FLU | SIIPSGPLK | 5 | 0.25 | Y |
| | (SEQ ID | | | |
| | NO: 1389) | | | |
| HIV NEF | AVDLSHFLK | 20.31 | 0.25 | Y |
| | (SEQ ID | | | |
| | NO: 1390) | | | |
| EBV | AVFDRKSDAK | 55.63 | 0.5 | Y |
| | (SEQ ID | | | |
| | NO: 1391) | | | |
| HBV core | YVNVNMGLK | 69.82 | 0.5 | Y |
| antigen | (SEQ ID | | | |
| | NO: 1392) | | | |
| HCV | RVCEKMALY | 304.91 | 1.3 | |
| | (SEQ ID | | | |
| | NO: 1393) | | | |
| CMV | KLGGALQAK | 736.59 | 1.6 | |
| | (SEQ ID | | | |
| | NO: 1394) | | | |

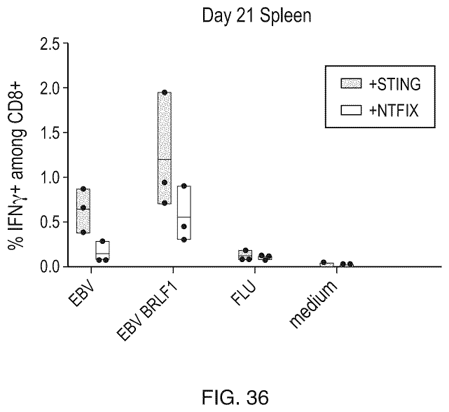

Day 21 spleen cells were restimulated ex vivo with the individual A*11 viral epitopes, followed by ICS (IFN-γ and TNF-α), to detect antigen-specific CD8+ T cell responses. Antigen-specific CD8+ T cell responses were observed for four out of the eight viral epitopes (EBV, EBV BRLF1, FLU and HIV NEF) and, as shown in FIG. 36, STING potentiated T cell responses for three of these viral epitopes (EBV, EBV BRLF1 and FLU).

Accordingly, the results described herein for HLA*A11 transgenic mice demonstrate that STING immunopotentiates antigen-specific T cell anti-KRAS responses, as well as anti-viral responses to other A11-restricted viral antigens, and is able to immunopotentiate responses to vaccine antigens in various formats (monomers and concatemers).

### Example 14: Immunopotentiation of STING is Reconstituted by Activation of Type 1 Interferon and NFκB

In this example, the HPV vaccine mouse model system was used to compare the immunopotentiation effect of STING to that of immune potentiators that either activate Type 1 interferon (constitutively active IRF3 and IRF7) or activate NFκB (constitutively active IKKβ). The STING mRNA construct (V155M mutation) is described in Example 1. The constitutively active IRF3 and IRF7 mRNA constructs are described in Example 2. The constitutively active IKKβ construct is described in Example 3. The HPV vaccine mouse model system is described in Example 5. Mice were immunized with the HPV vaccine in combination with either: (i) a control construct (NTFIX), (ii) the STING construct, (iii) the IRF3/IRF7 constructs, or (iv) the IRF3/IRF7/IKKβ constructs.

Day 21 spleen cells from mice in each test group were restimulated ex vivo for 4 hours at 37 degrees C. in the presence of GolgiPlug™ (containing Brefeldin A; BD Biosciences) with either E7 single peptides (3 individual peptides) or an E7 peptide pool, as described in Example 5. CD8 vaccine responses were assessed by intracellular staining (ICS) for IFN-γ or TNF-α. Representative ICS results for E7-specific responses by day 21 spleen cells for IFN-γ and TNF-α are shown in FIG. 37A (IFN-γ) and FIG. 37B (TNF-α). The experiment was carried out through 50 days, with the day 50 spleen cells results being comparable to the results observed at day 21. The results demonstrate that the combination of constitutively active IRF3+constitutively active IRF7+constitutively active IKKβ recapitulated the STING-mediated adjuvant phenotype. Thus, these results demonstrate that the immune potentiating potency of STING can be reconstituted by use of constructs that activate Type 1 Interferon and NFkB.

### Example 15: Immunopotentiation By Modulation of Intracellular Pathways

In this example, the immunopotentiation effect of STING was compared to that of immune potentiators that modulate intracellular pathways. Immune potentiator mRNA constructs encoding TAK1, TRAM or MyD88, each of which is an intracellular signaling protein that operates downstream of TLRs, were tested. The constitutively active STING construct (V155M) is described in Example 1. A representative amino acid sequence encoded by a TAK1 construct is shown in SEQ ID NO: 164 (encoded by the exemplary nucleotide sequences shown in SEQ ID NOs: 1411 and 1482). A representative amino acid sequence encoded by a TRAM construct is shown in SEQ ID NO: 136 (encoded by the exemplary nucleotide sequences shown in SEQ ID NOs: 1410 and 1481). Representative amino acid sequences encoded by MyD88 constructs are shown in SEQ ID NO: 134 (encoded by the exemplary nucleotide sequences shown in SEQ ID NOs: 1409 and 1480) and SEQ ID NO: 135. Mice were immunized with mRNA encoding ovalbumin as a test antigen in combination with an mRNA construct encoding either: (i) STING, (ii) TAK1, (iii) TRAM, or (iv) MyD88. The OVA antigen mRNA construct and the immune potentiator mRNA construct were coformulated in lipid nanoparticles comprising: Compound 25: Cholesterol: DSPC: PEG-DMG, at ratios of 50:38.5:10:1.5, respectively. Mice were immunized intramuscularly on days 1 and 15 at 0.5 mg/kg.

Day 25 spleen cells from mice in each test group were restimulated ex vivo for 4 hours at 37 degrees C. in the presence of GolgiPlug™ (containing Brefeldin A; BD Biosciences) with an OVA peptide (MHC Class I). CD8 vaccine responses were assessed by intracellular staining (ICS) for IFN-γ, TNF-α or IL-2. Representative ICS results for OVA-specific responses by day 25 spleen cells for IFN-γ, TNF-α and IL-2 are shown in FIG. 38A (IFN-γ), FIG. 38B (TNF-α) and FIG. 38C (IL-2). The experiment was carried out through 50 days, with the day 50 spleen cells results being comparable to the results observed at day 25. The results demonstrate that the TAK1, TRAM and MyD88 constructs showed immunopotentiating activity similar to STING. Thus, these results demonstrate that immune responses can be potentiated by modulating intracellular pathways using mRNA constructs encoding components of such intracellular pathways, in particular components that function downstream of TLRs.

### Example 16: Immunopotentiation By Adaptor Proteins and By Inflammasome or Necroptosome Induction

In this example, the immune potentiation ability of a panel of mRNA constructs was compared in mice using ovalbumin as a test antigen (as described in Example 15). The panel of mRNA constructs encoded either the adaptor proteins STING or MAVS (mitochondrial antiviral signaling protein), constitutively active IKKβ (which activates NFκB), caspases 1/4 (involved in inflammasome induction) or MLKL (involved in necroptosome induction). The constitutively active STING construct (V155M) is described in Example 1. The constitutively active IKKβ construct is described in Example 3 and encodes the amino acid sequence shown in SEQ ID NO: 152 (encoded by the exemplary nucleotide sequences shown in SEQ ID NOs: 153 and 1397). A representative amino acid sequence encoded by a MAVS construct is shown in SEQ ID NO: 1387 (encoded by the exemplary nucleotide sequences shown in SEQ ID NOs: 1413 and 1484). Representative amino acid sequence encoded by MLKL constructs are shown in SEQ ID NOs: 1327 (encoded by the exemplary nucleotide sequences shown in SEQ ID NOs: 1412 and 1483) and 1328. Representative amino acid sequences encoded by caspase-1 constructs are shown in SEQ ID NOs: 175-178 (encoded by the exemplary nucleotide sequences shown in SEQ ID NOs: 1395 and 1467). Representative amino acid sequences encoded by caspase-4 constructs are shown in SEQ ID NOs: 1352-1356 (encoded by the exemplary nucleotide sequences shown in SEQ ID NOs: 1396 and 1468). Mice were immunized with mRNA encoding ovalbumin as a test antigen in combination with an mRNA construct encoding either: (i) STING; (ii) MAVS; (iii) IKKβ; (iv) Caspase 1/4+IKKβ; (v) MLKL; or (vi) MLKL+STING. The NTFIX construct and DMXAA (a chemical activator of STING-dependent innate immunity pathways) were used as controls. The OVA antigen mRNA construct and the immune potentiator mRNA construct were coformulated in lipid nanoparticles comprising: Compound 25: Cholesterol: DSPC: PEG-DMG, at ratios of 50:38.5:10:1.5, respectively. Mice were immunized intramuscularly on days 1 and 15 at 0.5 mg/kg.

Spleen cells from mice in each test group were restimulated ex vivo for 4 hours at 37 degrees C. in the presence of GolgiPlug™ (containing Brefeldin A; BD Biosciences) with an OVA peptide (MHC Class I). Antigen-specific CD8 responses were assessed by intracellular staining (ICS) for IFN-γ. Representative ICS results for OVA-specific responses by day 21 spleen cells for IFN-γ are shown in FIG. 39. Representative ICS results for OVA-specific responses by day 50 spleen cells for IFN-γ are shown in FIG. 40. The results demonstrate that the adaptor compounds (STING and MAVS), induction of the inflammasome (by Caspase 1/4+IKKβ) or induction of the necroptosome (by MLKL) all resulted in immunopotentiation of antigen-specific CD8 responses. Furthermore, the combination of MLKL and STING exhibited enhanced activity as compared to MLKL alone. Moreover, the day 50 results demonstrate the immune potentiation effect was durable. These results demonstrate that immune responses can be potentiated by adaptor proteins, by induction of the inflammasome or by induction of the necroptosome.

### Example 17: Comparison of Constitutively Active STING Constructs

In this example, C₅₇/B16 mice were immunized with an OVA antigen-encoding mRNA construct co-formulated or co-administered with different constitutively active STING mutant mRNA constructs. The constitutively active STING constructs tested were: (i) p23 (V155M); (ii) p57 (R284M/V147L/N154S/V155M); (iii) p56 (V147L/N154S/V155M); and (iv) p19 (R284M). All constructs were tested co-formulated with the OVA antigen construct. The p23 construct also was tested co-administered with the OVA antigen construct but formulated separately. Mice were immunized on day 1 and day 14.

On day 21, mice were sacrificed and IFN-γ expression by CD8+ T cells was assessed by ICS as described herein. The results are shown in FIG. 41A, which demonstrates that all constitutively active STING mutant constructs tested exhibited the ability to immunopotentiate the antigen-specific CD8+ T cell response to the OVA antigen in vivo (as compared to the NTFIX control). Additionally, the p23 construct immunopotentiated the antigen-specific CD8+ T cell responses both when it was coformulated with the OVA antigen construct and when it was co-administered with the OVA antigen construct but formulated separately. Furthermore, on day 90, mice were sacrificed and IFN-γ expression by CD8+ T cells was assessed by ICS as described herein. The results are shown in FIG. 41B, which demonstrates that the immune potentiating effect of the constitutively active STING mutant constructs is durable.

### Example 18: Role of CD4 and CD8 T Cells in STING-Mediated Immunopotentiation

In this example, CD4-depleted or CD8-depleted mice were used to evaluate the role of CD4+ or CD8+ T cells in STING-mediated immunopotentiation. In a first series of experiments, to deplete CD4 cells, mice were injected intraperitoneally with the anti-CD4 mAb GK1.5 on days −3, −1, 11 and 13 of the experiment. Depletion efficiency was confirmed by flow cytometry. Mice were vaccinated on days 1 and 15 with the HPV16 E6/E7 antigen vaccine coformulated with the STING construct (V155M) intramuscularly at a dosage of 0.5 mg/kg. The vaccine and STING mRNA constructs were coformulated in lipid nanoparticles comprising: Compound 25: Cholesterol: DSPC: PEG-DMG, at ratios of 50:38.5:10:1.5, respectively, at a 1:1 ratio.

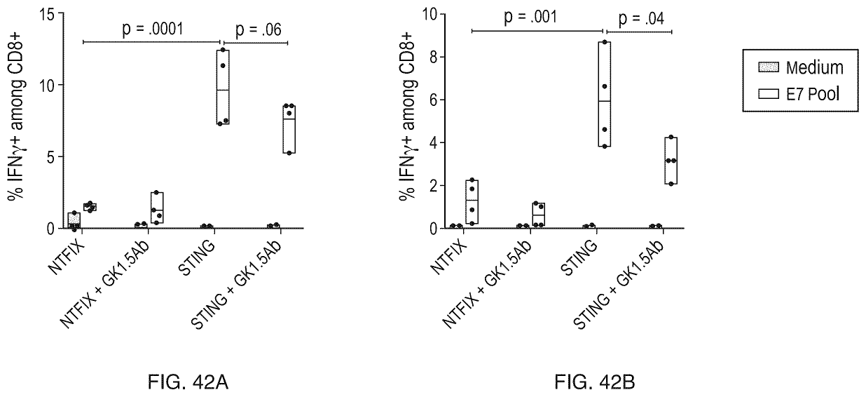

On days 21 and 50, mice were sacrificed and IFN-γ expression by CD8+ T cells was assessed by ICS as described herein. The results are shown in FIG. 42A (day 21) and FIG. 42B (day 50). Similar results were observed for TNF-α expression by CD8+ T cells as assessed by ICS (data not shown). The results demonstrate that the adjuvant effect mediated by STING is largely independent of CD4+ T cell help.

In a second series of experiments, the role of CD4 and CD8 T cells in the effect of the HPV-STING vaccine on tumor cell growth was examined using the TC1 model described in Example 10. TC1 HPV cells (2×10⁵ cells) were implanted subcutaneously into C₅₇/B6 mice and tumors were grown to a volume size of 100 mm³, which became day 1. On days 1 and 8, mice were administered the HPV16 E6/E7 soluble antigen vaccine coformulated with the STING construct (V155M) (1:1 ratio of sE6/E7 and STING) intramuscularly at a dosage of 10 μg. Control mice were treated with PBS only. Furthermore, on days 1, 4, 7, 10 and then biweekly until the end of the study, mice were treated with either anti-CD4 (GK1.5 mAb) or anti-CD8 (2.43 mAb) to deplete CD4 T cells or CD8 cells, respectively. Control mice were untreated with depleting antibody. The treatment groups and corresponding dosages are provided in Table 19.

TABLE 19

| Tumor size | | Vax dose 1 | Vax dose 2 | |
|---|---|---|---|---|
| (mm³) | Group | (day 1), μg | (day 8) μg | Treatment |
| 100 | PBS | none | none | none |
| | sE6/7 + STING | 10 | 10 | PBS |
| | (1:1) | | | |
| | sE6/7 + STING | 10 | 5 | anti-CD8 |
| | (1:1) | | | (2.43) |
| | sE6/7 + STING | 10 | 10 | anti-CD4 |
| | (1:1) | | | (GK1.5) |

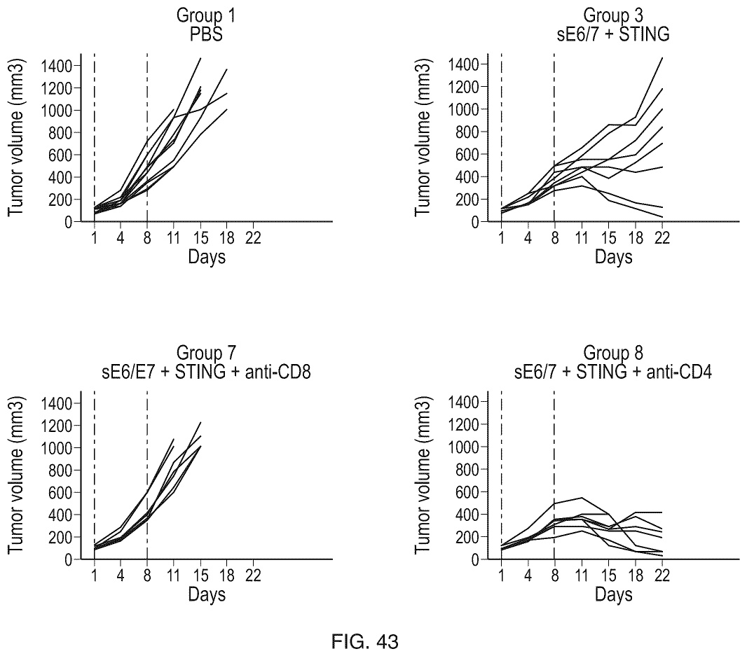

The results are shown in FIG. 43, which shows tumor volume over the course of 22 days. The results demonstrate that depletion of CD4+ T cells did not significantly affect the anti-tumor efficacy of the HPV-STING vaccine, whereas the depletion of CD8+ T cells did affect the anti-tumor efficacy of the HPV-STING vaccine, thereby demonstrating that the efficacy of the vaccine is dependent on CD8+ T cells but not on CD4+ T cells.

### Example 19: STING Skews CD8 Cells to Effector Memory Phenotype

In this example, to further confirm the results reported in Examples 5 and 6 regarding CD62L^{lo} effector memory cells, additional experiments were performed in which C₅₇/B16 mice were immunized with various concentrations of MC38 vaccine coformulated with various concentrations of STING immune potentiator mRNA construct. The amounts/ratios of Ag and STING used were the same as set forth in Table 15 of Example 11. Mice were immunized on days 1 and 14. On days 21 and 54, the percentage of CD62L10 effector memory cells among CD44^{hi}CD8+ cells was examined. The results are shown in FIG. 44A (day 21) and FIG. 44B (day 54). The results demonstrate that the STING immune potentiator mRNA construct skews the CD8 cell population to the effector memory phenotype (CD62L^{lo} cells).

### Example 20: STING Immunopotentiates Responses to a Concatemeric Vaccine at a Variety of Different Antigen: STING Ratios

In this example, whether an immune potentiator, such as constitutively active STING, can boost T-cell responses to a concatemeric vaccine was investigated. An mRNA construct encoding the CA-132 concatemer (described in Example 11), which encodes Class I and Class II epitopes, was used as the vaccine and the effect of the mRNA STING construct on T-cell responses to Class I and Class II epitopes was investigated. The CA-132 and STING mRNAs were either coformulated and delivered simultaneously, or were not coformulated, with a delayed delivery of STING mRNA. Animals were given a priming dose on day 1 and a boost on day 15. Splenocytes were harvested on day 21.

Different materials were tested in order to determine the immunogenicity when adding STING at various ratios to a concatemeric vaccine, to compare STING to top-ranked commercially available adjuvants, to determine whether the immunogenicity is dependent upon the timing of STING dosing, and to examine the immunogenicity of unformulated mRNA when dosed with STING. The following materials/conditions were tested: CA-132 (3 μg), CA-132 (3 μg) with Poly I:C (10 μg), CA-132 (3 μg) with MPLA (5 μg), STING (1 μg)/CA-132 (3 μg), STING (0.6 μg)/CA-132 (3 μg), STING (0.6 μg)/CA-57 (3 μg), STING (0.6 μg)/CA-132 (3 μg) (24 hours later), STING (0.6 μg)/CA-132 (3 μg) (48 hours later), STING (0.6 μg)/CA-132 (3 μg) (unformulated), and STING (6 μg)/CA-132 (30 μg) (unformulated). CA-57 is a concatemer of 5 Class II epitopes (all of which are contained within CA-132).

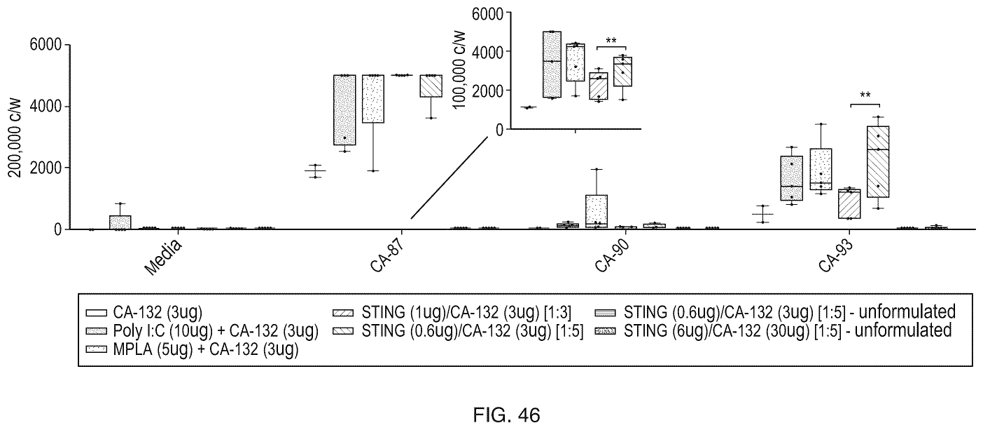

Results are shown in FIGS. 45-47. When the antigen-specific IFN-γ responses were examined with Class II epitopes, STING was found to boost the immune response to the MHC class II epitopes encoded by mRNA. STING behaved comparably to commercially available adjuvants (5-10 fold difference in dose). Although both ratios tested worked, the 1:5 STING:antigen ratio performed better than 1:3 combination (FIG. 45). Similar results were obtained using Class I epitopes as described above and shown in FIG. 46. Likewise, the 1:5 STING:antigen ratio was found to perform better than the 1:3 combination for class I epitopes.

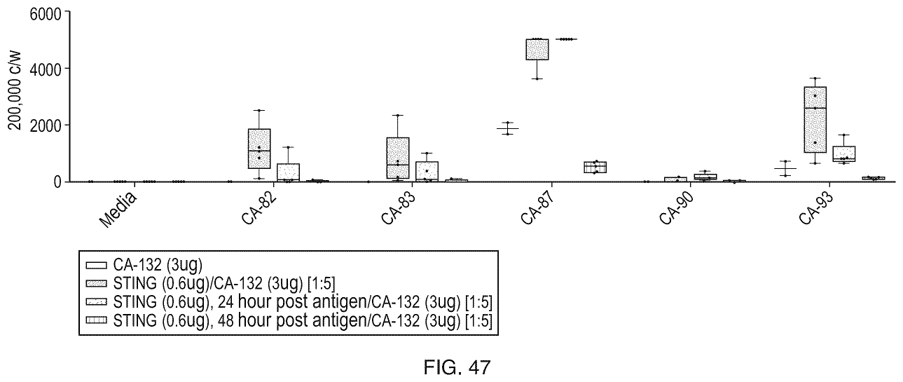

Further, it was found that dosing STING at a later time point (24 hours) produced similar increases in immunogenicity to codelivery (FIG. 47).

In a further experiment, the effect of different STING:antigen ratios was examined using a 52 murine epitopes. Mice received a prime dose on day 1, a boost dose on day 8, and splenocytes were harvested on day 15. T cell responses to re-stimulation were evaluated using ELISpot and FACS. Restimulation of T cells in vitro was with peptides sequences corresponding to epitopes encoded within the concatemer. T cell responses to two Class II epitope peptides (CA-82 and CA-83) and four Class I epitope peptides (CA-87, CA-93, CA-113 and CA-90) were examined.

Quite surprisingly, it was found that the addition of STING across the majority of ratios tested improved T cell responses compared to antigen alone and never performed worse than antigen alone. The breadth of responsiveness was unexpected. For four of the six antigens (epitopes) tested, the addition of STING to antigen at the 10-30 μg total dose consistently produced higher T cell responses than that of the 50 μg dose of antigen alone. Thus, there is a wide bell curve in the ratio of STING:antigen for improved immunogenicity.

The study groups were as shown below in Table 20.

TABLE 20

| | STING:Ag Ratio | | | | | | | | | | | |
|---|---|---|---|---|---|---|---|---|---|---|---|---|
| Total | 0:1 | | 20:1 | | 5:1 | | 1:1 | | 1:5 | | 1:20 | |
| mRNA | NTFIX | AG | STING | AG | STING | AG | STING | AG | STING | AG | STING | AG |
| (μg) | (μg) | (μg) | (μg) | (μg) | (μg) | (μg) | (μg) | (μg) | (μg) | (μg) | (μg) | (μg) |
| 0.15 | 2.85 | 0.15 | | | | | | | | | | |
| 0.5 | 9.5 | 0.5 | | | | | | | | | | |
| 1.5 | 28.6 | 1.5 | | | | | | | | | | |
| 3 | 27 | 3 | 2.85 | 0.15 | 2.4 | 0.6 | 1.5 | 1.5 | 0.6 | 2.4 | 0.15 | 2.85 |
| 10 | 20 | 10 | 9.5 | 0.5 | 8.3 | 1.4 | 5.0 | 5.0 | 1.4 | 8.3 | 0.5 | 9.5 |
| 30 | 0 | 30 | 28.6 | 1.4 | 25.0 | 4.2 | 15.0 | 15.0 | 4.2 | 25.0 | 1.4 | 28.6 |
| 50 | 0 | 20 | | | | | | | | | | |

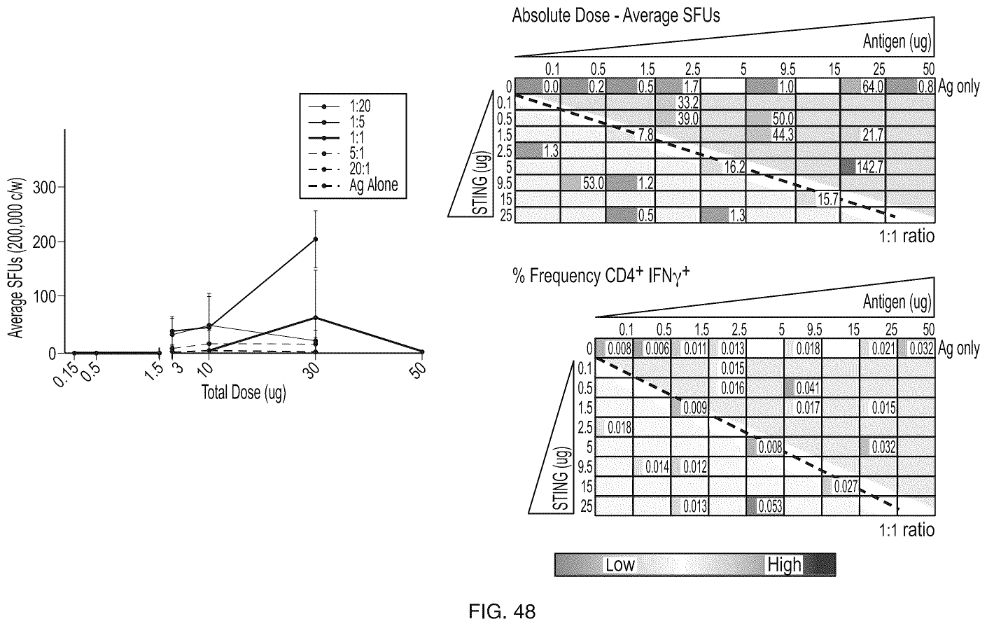

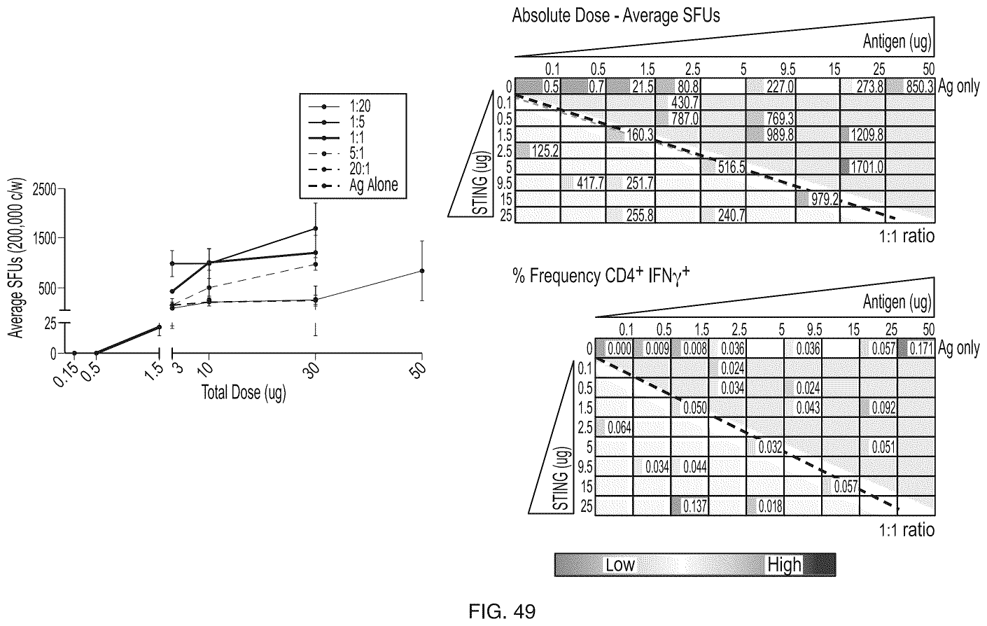

Among the Class II epitopes, CA-82 (results shown in FIG. 48) and CA-83 (results shown in FIG. 49) showed that adding STING increased T cell responses at ratios less than 1:1 (STING:antigen) relative to the antigen only group, including at doses up to 50 μg antigen alone. The left panel of FIG. 49 shows that adding STING increased T cell response at all ratios relative to the antigen only group.

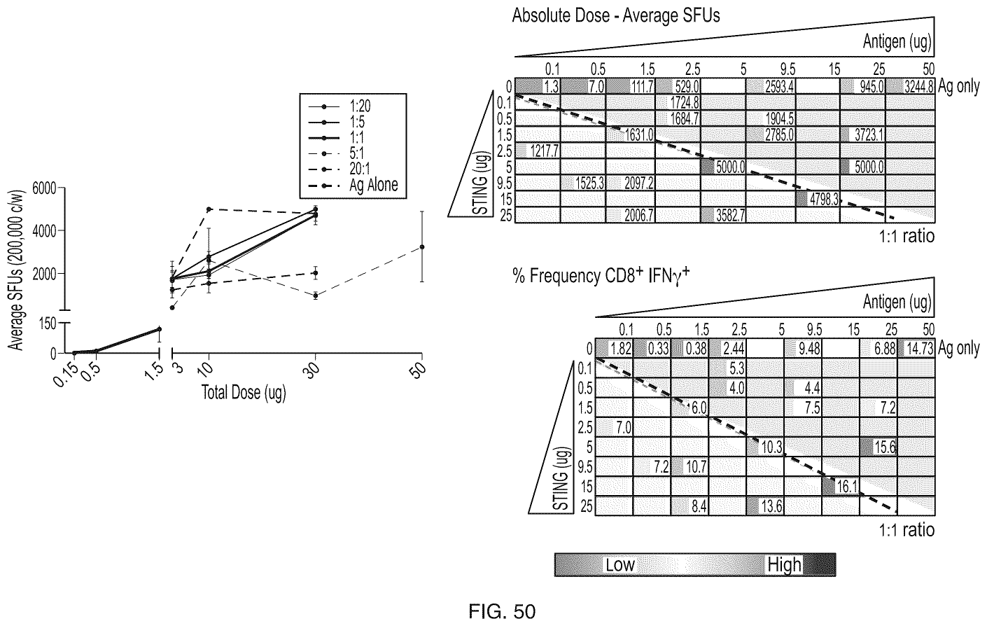

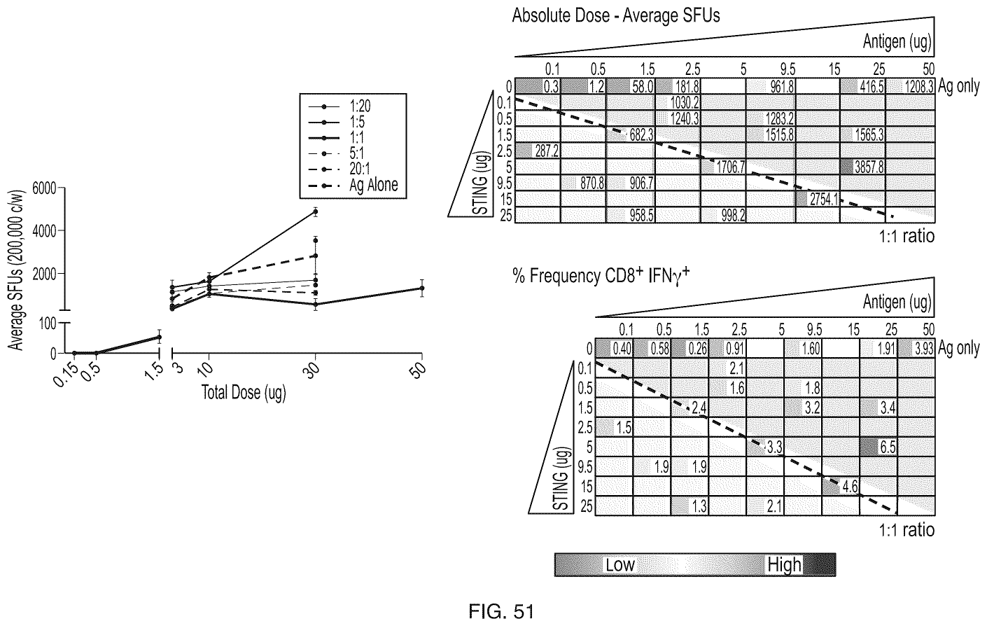

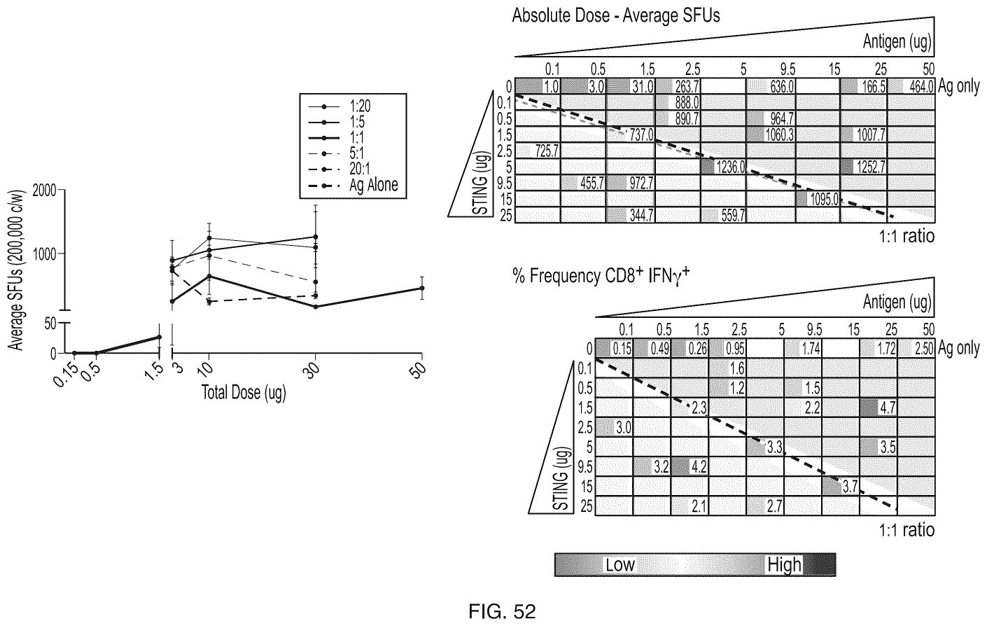

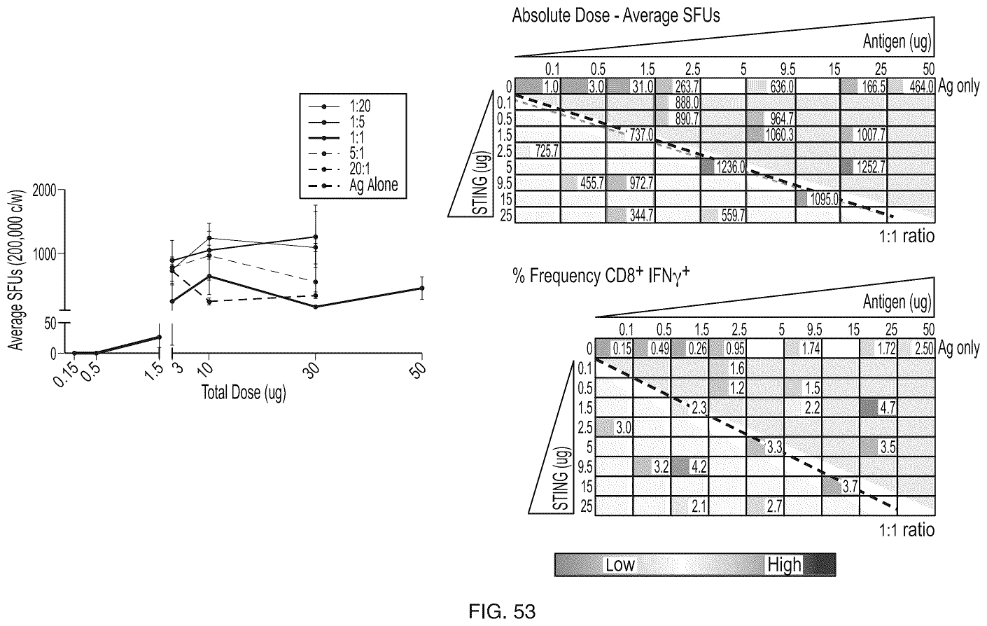

Similar results were seen with the Class I epitopes. CA-87 (results shown in FIG. 50), CA-93 (results shown in FIG. 51), CA-113 (results shown in FIG. 52), and CA-90 (results shown in FIG. 53) all showed that ratios of STING:antigen produced higher T cell responses relative to the antigen only group when compared to the total mRNA dose.

### Example 21: Fold Increase of STING-Mediated Immunopotentiation

In this example, to examine the magnitude of immune potentiation mediated by STING for a variety of antigens, mice were treated with STING in combination various antigens. In a first series of experiments, mice were treated with STING in combination with one of the following previously-described antigens: (i) HPV16 E7 (intracellular); (ii) HPV16 E7 (soluble); (iii) MC38 ADR neoantigen (intracellular); or (iv) OVA (soluble). 2.5 μg of the HPV16 E7 antigens or 5 μg of the MC38 ADR neoantigen was administered with 5 μg of STING. HPV16 E7 was co-formulated with E6, resulting in a 1:1 antigen:STING ratio for both the HPV and ADR antigens. On days 21 and 50+, spleen cells were harvested and T cells expressing IFN-γ were assessed by either intracellular staining (ICS) as described herein. The results were calculated as the fold-increase in immune responsive and are summarized below in Table 21 (day 21 results) and Table 22 (day 50+ results).

TABLE 21

| | | Fold increase (d 21) | | | |
|---|---|---|---|---|---|
| Antigen | Format | Exp 1 | Exp 2 | Exp 3 | average |
| HPV16 E7 | intracellular | 33.90 | 22.56 | | 28.23 |
| HPV16 E7 | soluble | 2.50 | 3.98 | 6.31 | 4.26 |
| MC38 ADR | intracellular | 4.57 | 11.67 | | 8.12 |
| neoantigen | | | | | |
| OVA | soluble | 18.12 | 3.67 | 7.57 | 9.79 |

TABLE 22

| | | Fold change (d 50+) | | | |
|---|---|---|---|---|---|
| Antigen | Format | Exp 1 | Exp 2 | Exp 3 | average |
| HPV16 E7 | intracellular | 13.33 | | | 13.33 |
| HPV16 E7 | soluble | 3.60 | 4.11 | 4.58 | 4.10 |
| MC38 ADR | intracellular | 27.80 | 86.00 | | 56.90* |
| neoantigen | | | | | |
| OVA | soluble | 29.85 | 4.93 | | 17.39 |

*= day 35

In a second series of experiments, mice were treated with STING in combination with the CA-132 concatemer vaccine described in Example 20 and antigen-specific T cells responses to various epitopes within the concatemer vaccine were assessed by ELISpot analysis for IFN-γ expression. The results were calculated as the fold-increase in immune responsive and are summarized below in Table 23

TABLE 23

| Epitope | Range of fold increase |
|---|---|
| CA-82 | 0.3-318 |
| CA-83 | 1.7-78 |
| CA-87 | 0.7-974 |
| CA-93 | 1.3-1148 |
| CA-113 | 1.5-725 |

The results demonstrate that while the fold-increase in immunoresponsiveness mediated by STING varied based on the antigen, for most antigens tested STING induced at least a 2-fold increase in immune responsiveness and for certain antigens exhibited even greater enhancement of immune responsiveness (e.g., more than 5-fold, more than 10-fold, more than 20-fold, more than 30-fold, more than 50-fold or more than 75-fold enhancement) relative to antigen alone (i.e., antigen+NTFIX mRNA).

### Example 22: MLKL mmRNA Constructs Induce Cell Death

In this example, a series of mmRNA constructs that encoded amino acid residues 1-180 of human or mouse MLKL were made and tested for their ability to induce cell death. These constructs typically also encoded an epitope tag at either the N-terminus or C-terminus to facilitate detection. Different epitope tags were tested (FLAG, Myc, CT, HA, V5). Additionally, all constructs contained a Cap 1 5′ Cap (7 mG (5′)ppp(5′) NlmpNp), a 5′ UTR, a 3′ UTR, a poly A tail of 100 nucleotides and were fully modified with 1-methyl-pseudouridine (m1ψ). In certain constructs, the 3′ UTR included miR-122 and miR-142-3p binding sites. The amino acid sequences of the open reading frame (ORF) of the human and mouse MLKL 1-180 constructs without any epitope tag are shown in SEQ ID NOs: 1327 and 1328, respectively. Exemplary nucleotide sequences encoding the MLKL protein of SEQ ID NO: 1327 are shown in SEQ ID NOs: 1412 and 1483. Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

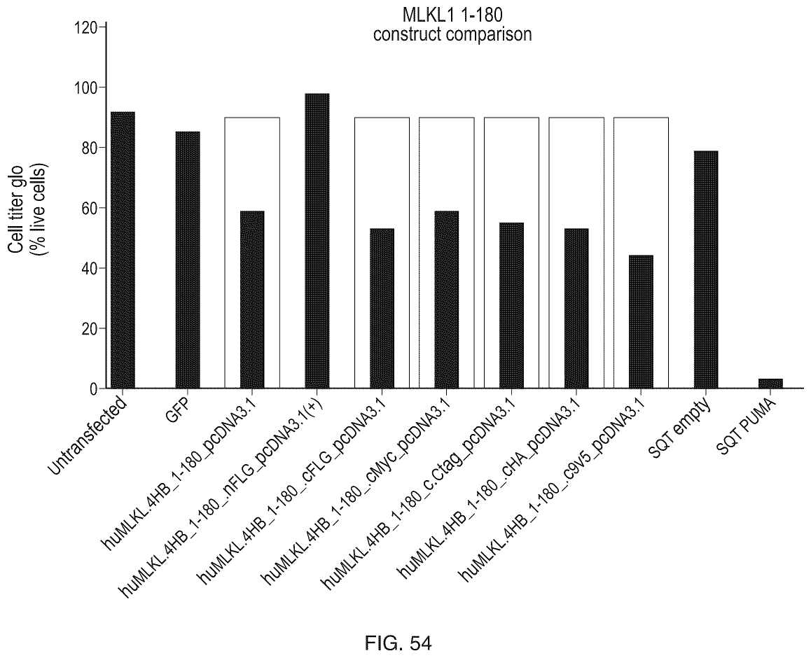

To determine wither the MLKL 1-180 constructs could induce cell death, the constructs were transfected into Hep3B human hepatoma cells. Twenty thousand HeLa cells/well were plated in 96 well plates and the mmRNA constructs were transfected into them using Lipofectamine 2000. After 24 hours, cell death was measured using the CellTiter-Glo® Luminescent Cell Viability Assay (Promega). The results are shown in FIG. 54, which demonstrates that the MLKL 1-180 mmRNA constructs were capable of inducing cell death of the HeLa cells.

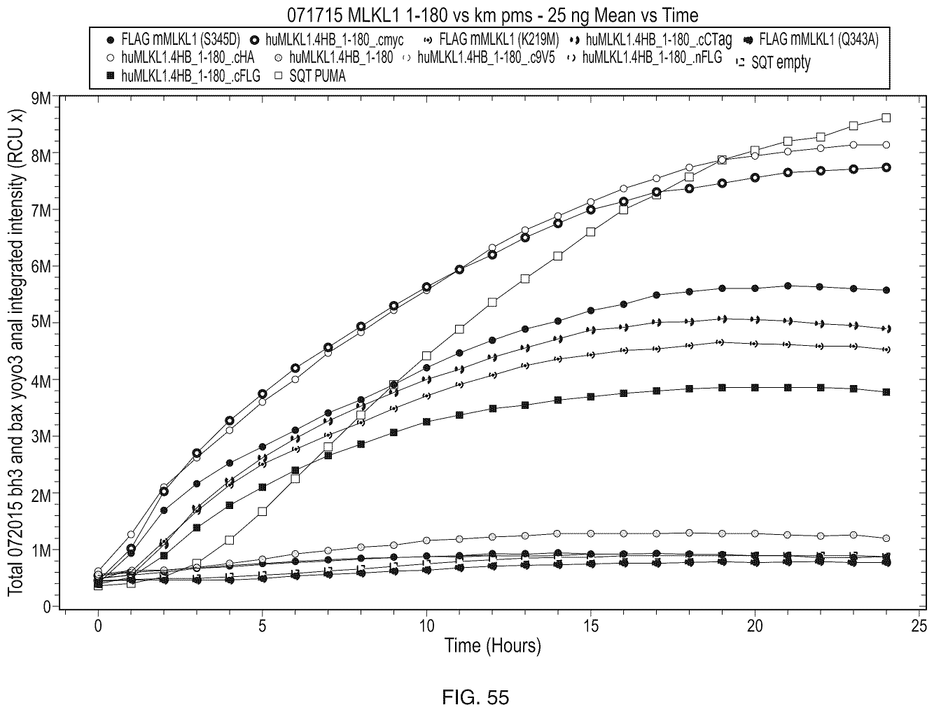

These results were confirmed by conducting similar experiments with the MLKL 1-180 mmRNA constructs and Hep3B cells in the presence of YOYO-3® (Life Technologies), a DNA dye that is taken up preferentially by dead cells that is used to measure the extent of cell death. The experiments conducted using the YOYO-3® read-out system for cell viability, the results of which are shown in FIG. 55, confirmed that the MLKL 1-180 mmRNA constructs were capable of inducing cell death of the Hep3B hepatoma cells.

### Example 23: MLKL mmRNA Constructs Cause Necroptosis

In the example, the ability of the MLKL 1-180 mmRNA constructs to cause necroptosis was examined. Necroptosis is characterized by rupture of the plasma membrane and leakage of the cytosolic contents into the surrounding area. This can be tested for in in vitro assay by detection of damage-associated molecular patterns (DAMPs) leaking into the culture medium.

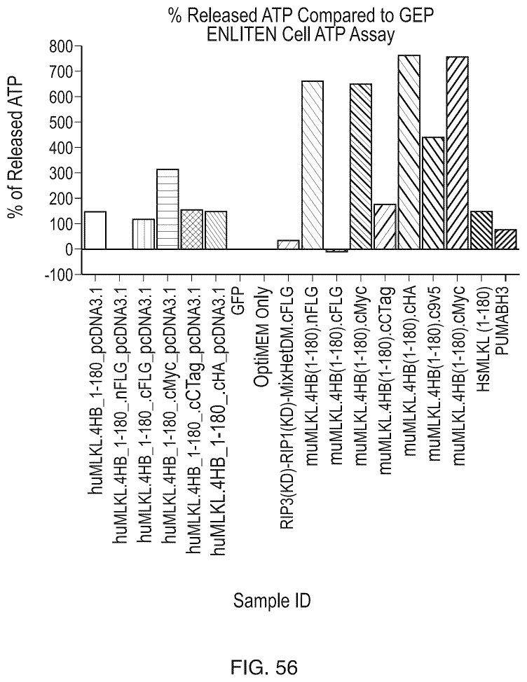

In a first series of experiments, the MLKL 1-180 mmRNA constructs were transfected into HeLa cells (as described in Example 22) and release of ATP, a DAMP, was measured as an indicator of necroptosis. Release of ATP was detected using the ENLITEN® ATP Assay (Promega). The results, which are shown in FIG. 56, demonstrate that the MLKL 1-180 mmRNA constructs induce the release of ATP from the cells, thereby indicating that the constructs are causing necroptosis.

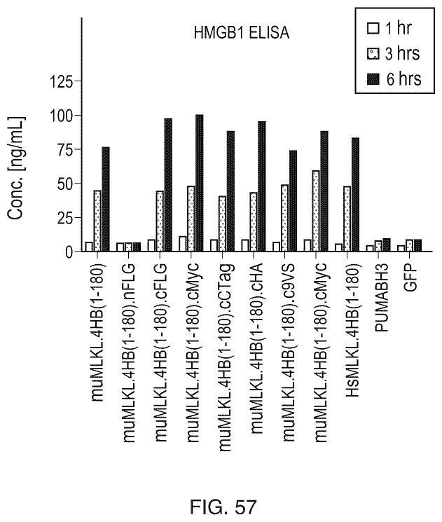

To confirm that necroptosis was occurring, a second series of experiments were performed in which the MLKL 1-180 mmRNA constructs were transfected into HeLa cells and release of HMGB1, another DAMP, was measured as an indicator of necroptosis. Release of HMGB1 was detected using an HMGB1 ELISA assay. For this set of experiments, HeLa cells (2×10⁴ cells/100 μl/well) were transfected with a transfection mixture (20 μl) containing mRNA construct (200 ng/well; 1 μl volume), Lipofectamine (0.2 μl/well volume) and Opti-MEM (18.8 μl/well volume). Prior to transfection of the cells, the transfection mixture was incubated for 20 minutes at room temperature and then the transfection mixture was added on top of the cells. The culture plates were tapped gently and then incubated at 37° C., 5% CO₂ for 0, 1, 3 and 6 hrs. At each of these time points, 110 μl supernatant was removed, pooled and spun down at 1000 rpm. 50 μl of supernatant per transfection was used in a standard HMGB1 ELISA. The results are shown in FIG. 57, which demonstrate that the MLKL 1-180 mmRNA constructs induce the release of HMGB1 from the cells, thereby indicating that the constructs are causing necroptosis.

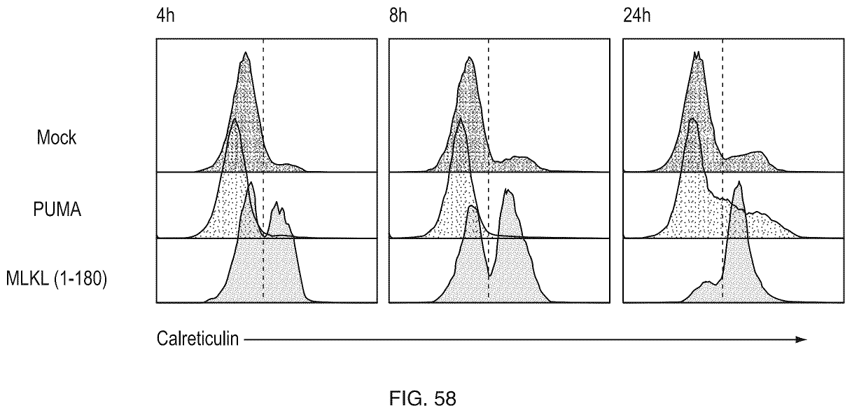

A third series of experiments examined the effect of treatment with an MLKL 1-180 mmRNA construct on cell surface expression of calreticulin (CRT), a DAMP molecule that is normally in the lumen of the endoplasmic reticulum but that translocates to the surface of dying cells after induction of necroptosis, where it mediates phagocytosis by macrophages and dendritic cells. Cells were either mock transfected, transfected with an apoptosis-inducing construct (“PUMA”) or transfected with an MLKL 1-180 mmRNA construct (huMLKL.4HB(1-180).cHA miR122/142-3p) and cell surface stained by standard methods for expression of calreticulin. The results are shown in FIG. 58, which demonstrates that the MLKL construct, but not the apoptosis-inducing construct, induced the translocation of CRT to the cell surface, thereby further confirming that the MLKL construct caused necroptosis.

A fourth series of experiments examined the effect of the inhibitor necrosulfonamide (NSA) on MLKL-induced cell death. NSA is an inhibitor that specifically targets MLKL. NSA was shown to inhibit cell death in a concentration dependent manner (measured using YOYO-3® as the read-out; data not shown) induced by the MLKL construct, thereby confirming that the observed cell death was necroptotic cell death induced by MLKL.

### Example 24: RIPK3 and GSDMD mmRNA Constructs Induce Cell Death

In this example, a series of mmRNA constructs that encoded RIP3K or GSDMD were made and tested for their ability to induce cell death. These constructs typically also encoded an epitope tag at either the N-terminus or C-terminus to facilitate detection. Different epitope tags were tested (FLAG, Myc, CT, HA, V5). Additionally, all constructs contained a Cap 1 5′ Cap (7 mG (5′)ppp(5′) NlmpNp), a 5′ UTR, a 3′ UTR, a poly A tail of 100 nucleotides and were fully modified with 1-methyl-pseudouridine (m1ψ). The ORF amino acid sequences of the RIP3K constructs without any epitope tag are shown in SEQ ID NOs: 1329-1344. Exemplary nucleotide sequences encoding the RIPK3 protein of SEQ ID NO: 1339 is shown in SEQ ID NOs: 1415 and 1486. The ORF amino acid sequences of the GSDMD constructs without any epitope tag are shown in SEQ ID NOs: 1367-1372. Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

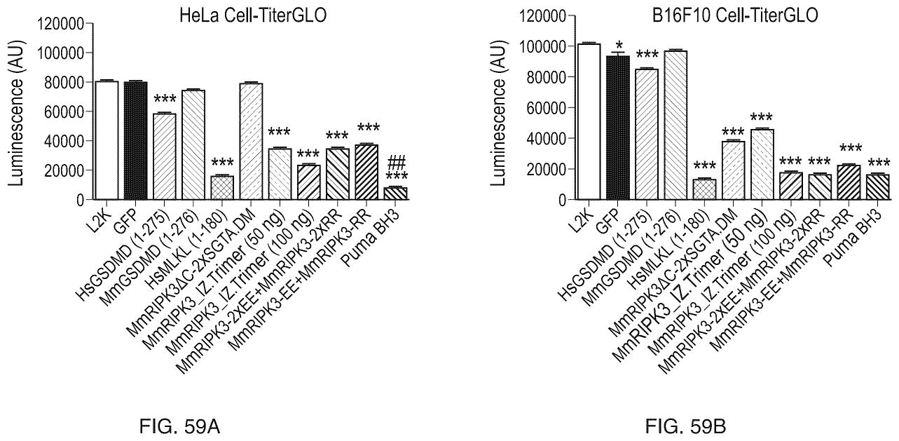

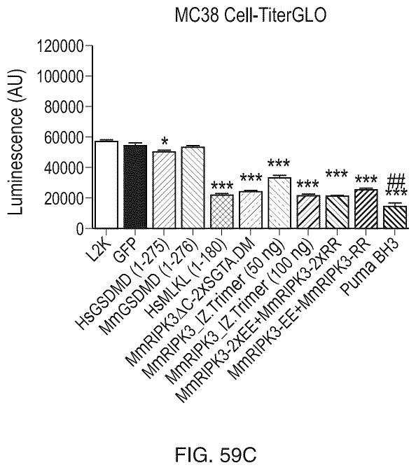

To determine wither the RIPK3 or GSDMD constructs could induce cell death, the constructs were transfected into three different cells types: HeLa cells, B16F10 cells and MC38 cells. Five thousand cells/well were plated in 96 well plates and the mmRNA constructs were transfected into them using Lipofectamine 2000. After 24 hours, cell death was measured using the CellTiter-Glo® Luminescent Cell Viability Assay (Promega). The results are shown in FIG. 59A (HeLa cells), FIG. 59B (B16F10 cells) and FIG. 59C (MC38 cells), with data for the MLKL (1-180) construct also shown for comparison purposes. The results demonstrate that the RIPK3 mmRNA constructs were capable of inducing cell death in all three cell types with a potency comparable to that observed for the MLKL (1-180) construct. The results further demonstrate that the GSDMD construct also was capable of inducing cell death in all three cell types, albeit with a lesser potency than that observed for the MLKL (1-180) construct. Similar results were observed for experiments conducted with the YOYO-3® read-out system.

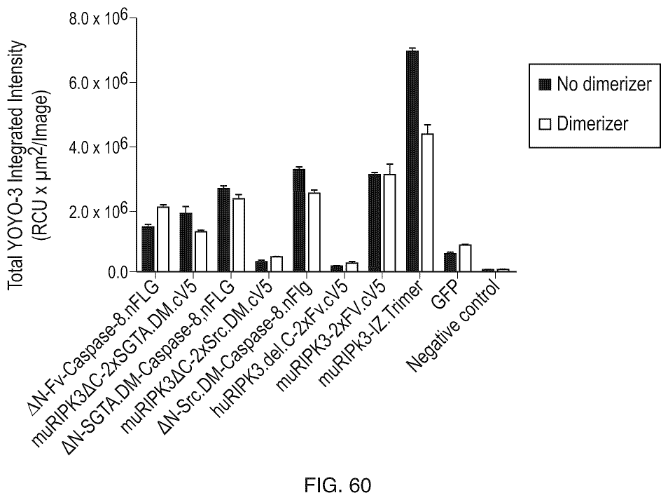

A series of additional RIPK3 constructs were made that were designed to oligomerize. These constructs contain protein domains (IZ trimer, or leucine zipper chiral domains (EE and RR)), which lead to trimerization and oligomerization of proteins. Induced dimer or trimer formation of RIPK3 leads to higher molecular weight oligomers and induction of necroptosis (see Yatim et al., Science, 2015 and Orozco et al, Cell Death Differ, 2014 for reference). These constructs were tested for their ability to induce cell death by transfection into NIH3T3 cells. Cell death was measured using the YOYO-3® read-out system at 15 hours post-transfection. The results are shown in FIG. 60 and Table 24, which demonstrates that the multimerizing RIPK3 constructs induce death of the NIH3T3 cells.

TABLE 24

| | Dimerize | |
|---|---|---|
| | with B/B | Binds to |
| Protein | homodimerizer | RIPK1 |
| ΔN-Fv-Caspase-8.nFLG | Yes | No |
| muRIPK3ΔC-2xSGTA.DM.cV5 | No | No |
| ΔN-SGTA.DM-Caspase-8.nFlg | No | No |
| muRIPK3ΔC-2xSrc.DM.cV5 | No | No |
| ΔN-Src.DM-Caspase-8.nFlg | No | No |
| huRIPK3.del.C-2xFv.cV5 | Yes | No |
| muRIPK3-2xFV.cV5 | Yes | Yes |
| muRIPK3-IZ Trimer | No | Yes |

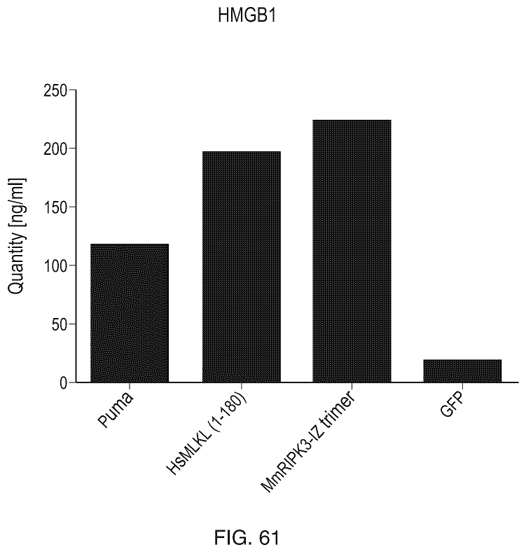

The ability of the multimerizing RIPK3 constructs to induce DAMP release was examined as an indicator of induction of necroptosis by the constructs. B16F10 cells were transfected with either a multimerizing RIPK3 construct (RIPK3-IZ trimer), an apoptosis-inducing construct (PUMA), an MLKL 1-180 construct (huMLKL.4HB (1-180).cHA miR122/142-3p) shown in Example 23 to induce DAMP release or a GFP control construct. Release of HMGB1 was detected using an HMGB1 ELISA assay. The results are shown in FIG. 61, which demonstrates that the multimerizing RIPK3 construct induced the release of HMGB1 at similar levels to the MLKL construct.

Another series of experiments examined the effect of the inhibitor GSK′872 on RIPK3-induced cell death. GSK′872 is an inhibitor that specifically targets RIPK3. GSK′872 was shown to inhibit cell death in a concentration dependent manner (measured using YOYO-3® as the read-out; data not shown) induced by RIPK3 constructs, thereby confirming that the observed cell death was necroptotic cell death induced by RIPK3.

### Example 25: DIABLO mmRNA Constructs Induce Cell Death

In this example, a series of mmRNA constructs that encoded DIABLO were made and tested for their ability to induce cell death. These constructs typically also encoded an epitope tag at either the N-terminus or C-terminus to facilitate detection. Different epitope tags were tested (FLAG, Myc, CT, HA, V5). Additionally, all constructs contained a Cap 1 5′ Cap (7 mG (5′)ppp(5′) NlmpNp), 5′ UTR, 3′ UTR, a poly A tail of 100 nucleotides and were fully modified with 1-methyl-pseudouridine (m1ψ). The ORF amino acid sequences of the DIABLO constructs without any epitope tag are shown in SEQ ID NOs: 165-172. Exemplary nucleotide sequences encoding the DIABLO protein of SEQ ID NO: 169 are shown in SEQ ID NOs: 1416 and 1487. Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

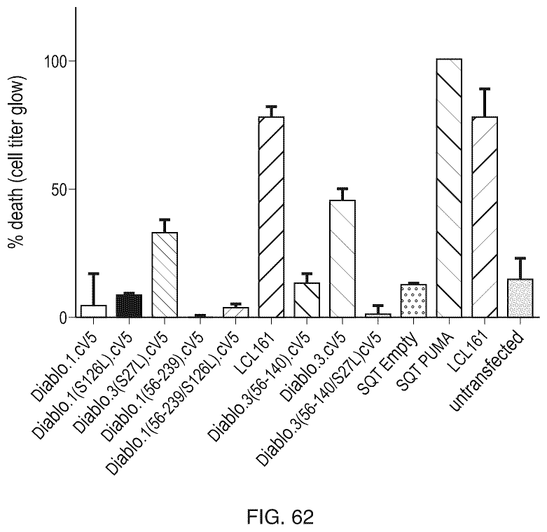

To determine wither the DIABLO constructs could induce cell death, the constructs were transfected into SKOV3 cells. Ten thousand cells/well were plated in 96 well plates and the mmRNA constructs were transfected into them using Lipofectamine 2000. After 41 hours, cell death was measured using the CellTiter-Glo® Luminescent Cell Viability Assay (Promega). The results are shown in FIG. 62, which demonstrate that a number of the DIABLO mmRNA constructs were capable of inducing cell death.

### Example 26: Caspase 4, Caspase-5, Caspase-11, Pyrin, NLRP3 and ASC mmRNA Constructs Induce Cell Death

In this example, mmRNA constructs encoding various forms of caspase-4, caspase-5, caspase-11, Pyrin, NLRP3 or ASC were prepared and transfected into cells to examine their ability to induce cell death using the YOYO-3® DNA dye (Life Technologies) to measure the extent of cell death.

In a first series of experiments, a panel of mmRNA constructs that encoded various caspase-4, -5 or -11 proteins were made and tested for their ability to induce cell death. Constructs tested encoded either (i) full-length wild-type caspase-4, caspase-5 or caspase-11; (ii) full-length caspase-4, -5 or -11 plus an IZ domain; (iii)N-terminally deleted caspase-4, -5 or -11 plus an IZ domain; (iv) full-length caspase-4, -5 or -11 plus a DM domain; or (v)N-terminally deleted caspase-4, -5 or -11 plus a DM domain. The N-terminally deleted forms of caspase-4 and caspase-11 contained amino acid residues 81-377, whereas the N-terminally deleted form of caspase-5 contained amino acid residues 137-434. These constructs typically also encoded an epitope tag (e.g., FLAG, Myc, CT, HA, V5) at either the N-terminus or C-terminus to facilitate detection. Additionally, all constructs contained a Cap 1 5′ Cap (7 mG (5′)ppp(5′) NlmpNp), a 5′ UTR, a 3′ UTR, a poly A tail of 100 nucleotides and were fully modified with 1-methyl-pseudouridine (m1ψ). The ORF amino acid sequences of the caspase-4 constructs without any epitope tag are shown in SEQ ID NOs: 1352-1356. The ORF amino acid sequences of the caspase-5 constructs without any epitope tag are shown in SEQ ID NOs: 1357-1361. The ORF amino acid sequences of the caspase-11 constructs without any epitope tag are shown in SEQ ID NOs: 1362-1366. Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

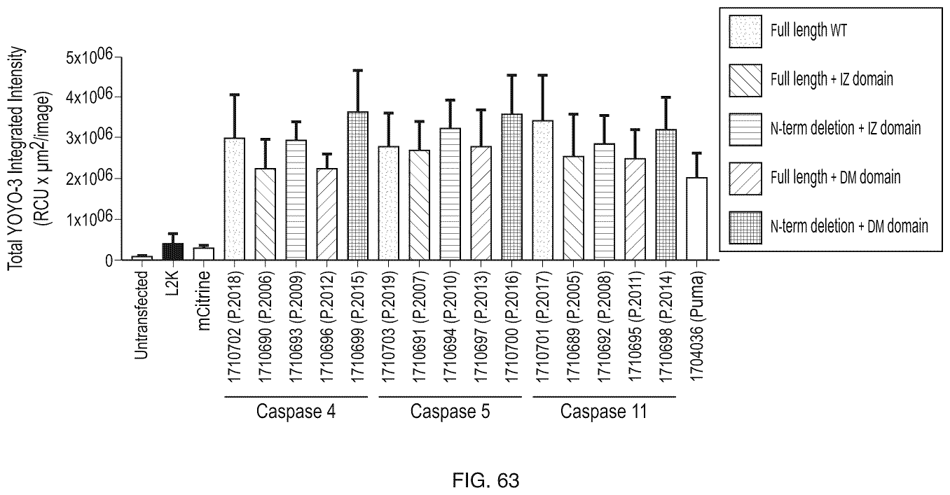

To determine whether the caspase-4, -5 and -11 constructs could induce cell death, the constructs were transfected into HeLa cells using Lipofectamine 2000. After 24 hours, cell death was measured using the YOYO-3® DNA dye. The results are shown in FIG. 63, which demonstrates that all five forms of the caspase-4, caspase-5 and caspase-11 mmRNA constructs were capable of inducing cell death of the HeLa cells.

In a second series of experiments, a panel of mmRNA constructs that encoded various Pyrin, NLRP3 or ASC proteins were made and tested for their ability to induce cell death. These constructs typically also encoded an epitope tag (e.g., FLAG, Myc, CT, HA, V5) at either the N-terminus or C-terminus to facilitate detection. Additionally, all constructs contained a Cap 15′ Cap (7 mG (5′)ppp(5′) NlmpNp), a 5′ UTR, a 3′ UTR, a poly A tail of 100 nucleotides and were fully modified with 1-methyl-pseudouridine (m1ψ). The ORF amino acid sequences of the Pyrin constructs without any epitope tag are shown in SEQ ID NOS: 1375 and 1376. The ORF amino acid sequences of the NLRP3 constructs without any epitope tag are shown in SEQ ID NOs: 1373 and 1374. The ORF amino acid sequences of the ASC constructs without any epitope tag are shown in SEQ ID NOs: 1377 and 1378. Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

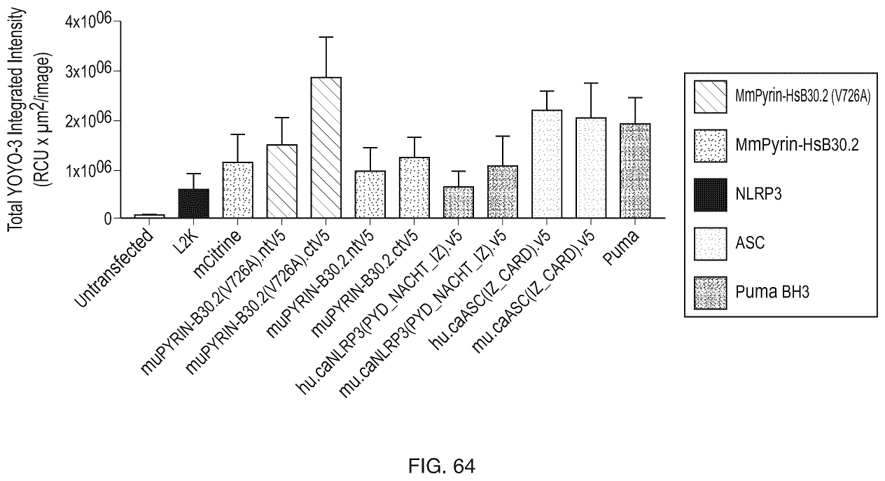

To determine whether the Pyrin, NLRP3 and ASC constructs could induce cell death, the constructs were transfected into HeLa cells using Lipofectamine 2000. After 24 hours, cell death was measured using the YOYO-3® DNA dye. The results are shown in FIG. 64, which demonstrates that the Pyring, NLRP3 and ASC mmRNA constructs were capable of inducing cell death of the HeLa cells.

### Example 27: Constitutively Active IRF3 and IRF7 mmRNA Constructs Activate an Interferon-Sensitive Response Element (ISRE)

In this example, a reporter gene whose transcription was driven by an interferon-sensitive response element (ISRE) was used to test the ability of constitutively active IRF3 and IRF7 mRNA constructs to activate the ISRE. Constitutively active IRF3 and IRF7 constructs were prepared and are described below. These constructs typically also encoded an epitope tag at either the N-terminus or C-terminus to facilitate detection. Different epitope tags were tested (FLAG, Myc, CT, HA, V5). Additionally, all constructs contained a Cap 15′ Cap (7 mG (5′)ppp(5′) NlmpNp), 5′ UTR, 3′ UTR, a poly A tail of 100 nucleotides and were fully modified with 1-methyl-pseudouridine (m1ψ). The ORF amino acid sequences of representative constitutively active mouse and human IRF3 constructs, comprising a S396D point mutation, without any epitope tag are shown in SEQ ID NOs: 11 and 12, respectively. Exemplary nucleotide sequences encoding these IRF3 proteins are shown in SEQ ID NOs: 210 and 211, respectively, and SEQ ID NOs: 1452 and 1453, respectively. The ORF amino acid sequences of representative constitutively active human IRF7 constructs without any epitope tag are shown in SEQ ID NOs: 13-20. Exemplary nucleotide sequences encoding these IRF7 proteins are shown in SEQ ID NOs: 212-219, respectively and SEQ ID NOs: 1454-1461. Exemplary 5′ UTRs for use in the constructs are shown in SEQ ID NOs: 21 and 1323. An exemplary 3′ UTR for use in the constructs is shown in SEQ ID NO: 22. An exemplary 3′ UTR comprising miR-122 and miR-142-3p binding sites for use in the constructs is shown in SEQ ID NO: 23.

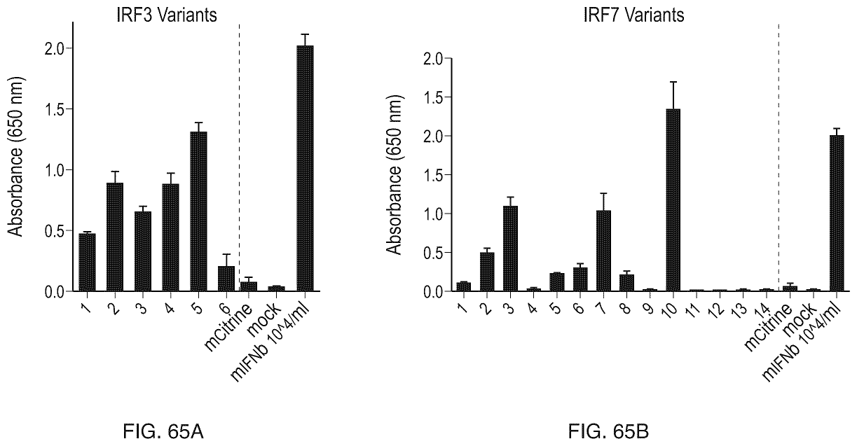

The results are shown in FIG. 65A-B, which demonstrate that the constitutively active IRF3 constructs (FIG. 65A) and the constitutively active IRF7 constructs (FIG. 65B) both stimulated reporter gene expression, thereby indicating that the constructs were capable of activating the interferon-sensitive response element (ISRE).

### Example 28: Effect of Priming on Release of Inflammatory Cytokines by Cells Treated with Pyroptotic Constructs

In this example, the effect of priming cells with an immune potentiator agent before transfection with a pyroptotic mRNA construct on release of proinflammatory cytokines by the cells was examined.

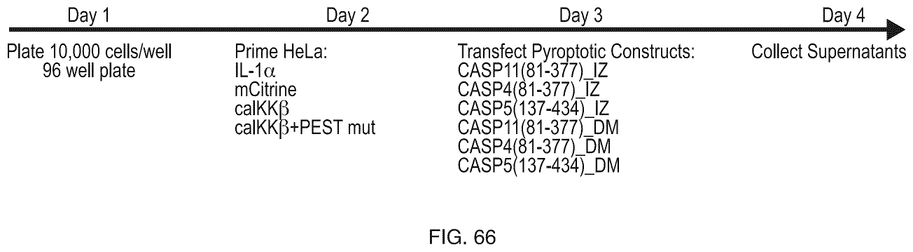

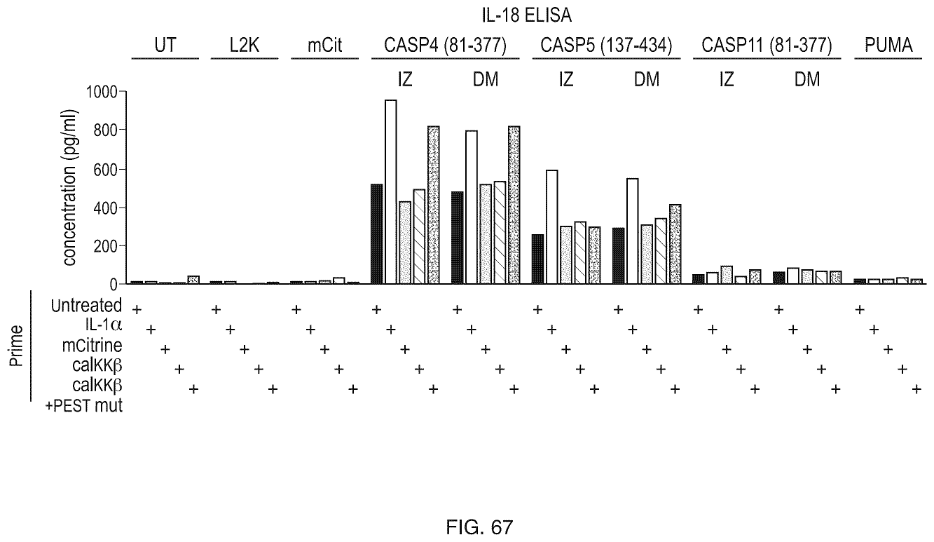

The design of the study is illustrated in FIG. 66. On Day 1, 10,000 HeLa cells/well were plated in 96 well plates. On Day 2, the cells were treated with one of the immune potentiator agents shown in FIG. 66 (the constitutively active IKKβ constructs are described further in Example 3). On Day 3, the cells were transfected with one of the caspase-4, caspase-5 or caspase-11 mRNA constructs shown in FIG. 66 (the caspase-4, -5 and -11 constructs are described further in Example 26). On Day 4, supernatants were collected and assayed for levels of the inflammatory cytokine IL-18 by standard ELISA. The results are shown in FIG. 67, which demonstrates that priming of the cells in particular with the immune potentiators IL-1α or the constitutively active IKKβ construct with PEST mutation stimulated release of proinflammatory cytokines by the HeLa cells, in particular those treated with the caspase-4 or caspase-5 constructs. These results demonstrate the benefit of combining an immune potentiator with an mRNA construct encoding a polypeptide that stimulates immunogenic cell death in order to enhance a proinflammatory response.

### Example 29: Anti-Tumor Effects of Executioner mRNAs, Alone or in Combination with an Immune Potentiator and/or Immune Checkpoint Inhibitor

In this example, the effect of executioner mRNAs on tumor growth in vivo in mice was examined. Executioner mRNA constructs encoding MLKL, RIPK3 or DIABLO were used alone or in combination, as well as in combination with an immune potentiator (STING mRNA construct) and/or an immune checkpoint inhibitor (anti-CTLA4 antibody or anti-PD-1 antibody).

In a first set of experiments, mice carrying MC38 colon carcinoma tumors (5×10⁵ cells implanted subcutaneously; tumors ˜ 100-120 mm³ in size at time of treatment) were divided into eleven treatment groups and treated intratumorally with the following mRNA constructs biweekly for 4 weeks (days 1, 4, 8, 11, 17, 20, 24 and 27), with certain groups also being treated with an immune checkpoint inhibitor(s) as indicated: (i) NT-MOD as a negative control; (ii) huMLKL.4HB (1-180).cHA miR122/142-3p (12.5 μg/animal); (iii) DIABLO (12.5 μg/animal); (iv) muRIPK3-IZ.Trimer (12.5 μg/animal); (v) huMLKL.4HB (1-180).cHA miR122/142-3p (12.5 μg/animal)+anti-CTLA4 9H10 (5 mg/kg, intraperitoneally on day 1, 2.5 mg/kg intraperitoneally on days 4 and 7); (vi) DIABLO (12.5 μg/animal)+anti-CTLA4 9H10 (5 mg/kg, intraperitoneally on day 1, 2.5 mg/kg intraperitoneally on days 4 and 7); (vii) muRIPK3-IZ.Trimer (12.5 μg/animal)+anti-CTLA4 9H10 (5 mg/kg, intraperitoneally on day 1, 2.5 mg/kg intraperitoneally on days 4 and 7); (viii) NT-MOD+anti-CTLA4 9H10 (5 mg/kg, intraperitoneally on day 1, 2.5 mg/kg intraperitoneally on days 4 and 7); (ix) huMLKL.4HB (1-180).cHA miR122/142-3p (12.5 μg/animal)+DIABLO (12.5 μg/animal); (x) huMLKL.4HB (1-180).cHA miR122/142-3p (12.5 μg/animal)+DIABLO (12.5 μg/animal)+anti-CTLA4 9H10 (5 mg/kg, intraperitoneally on day 1, 2.5 mg/kg intraperitoneally on days 4 and 7); and (xi) anti-CTLA4 9H10 (5 mg/kg, intraperitoneally on day 1, 2.5 mg/kg intraperitoneally on days 4 and 7)+anti-PD-1 RMP1-14 (5 mg/kg intraperitoneally biweekly for two weeks) as a positive control.

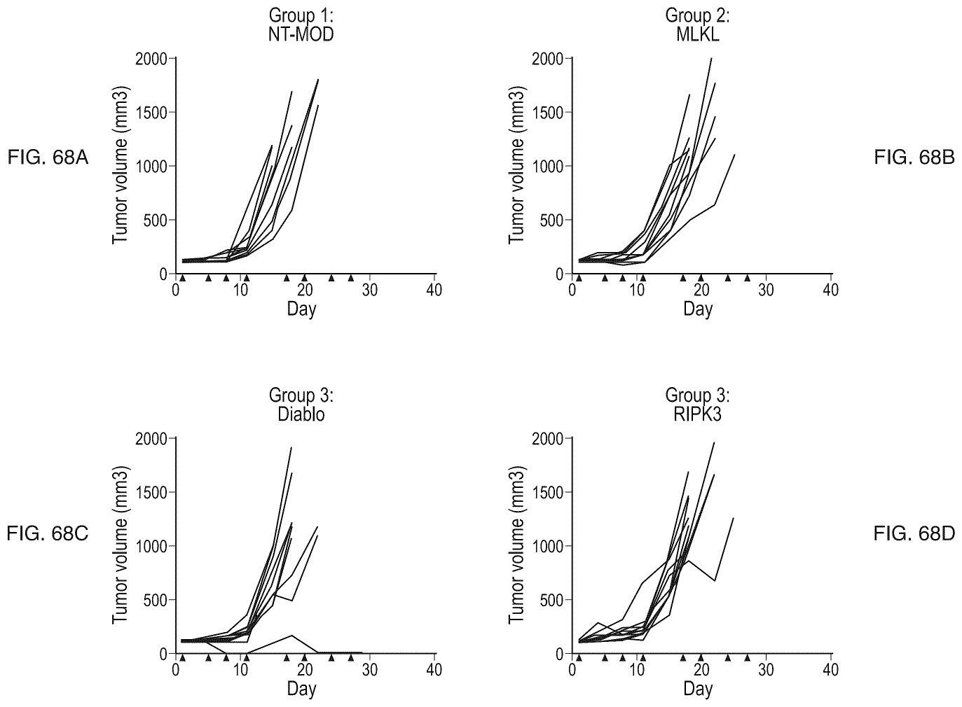

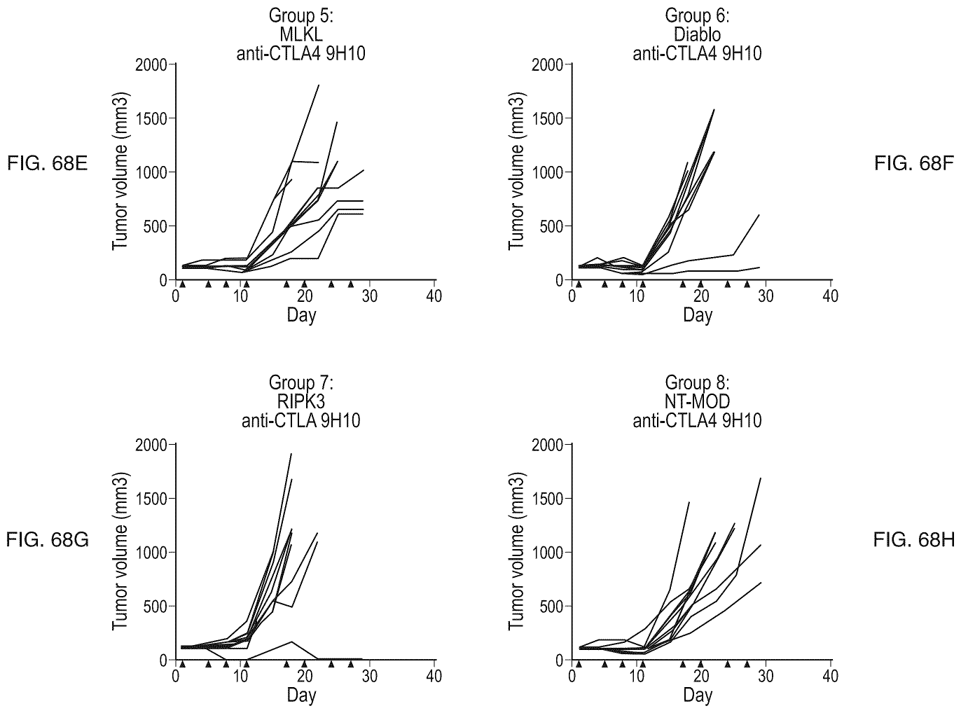

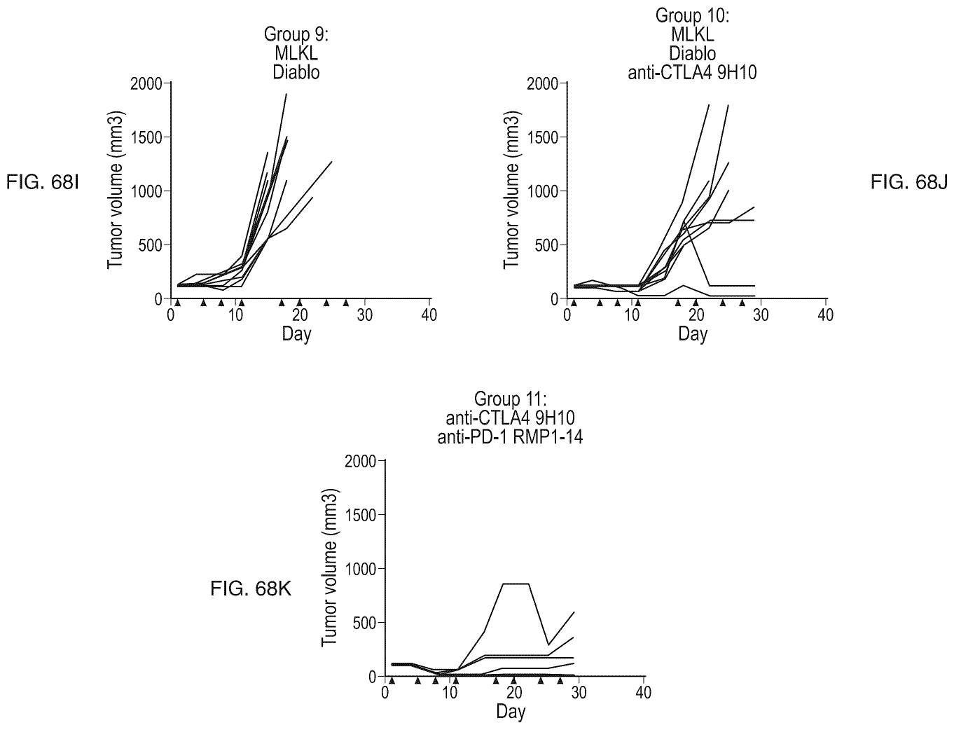

The results are shown in FIGS. 68A-68K, corresponding to the eleven treatment groups described above, showing tumor volume (mm³) in the mice over the time course of the experiment. Additionally, serum was collected at 10 hours and 24 hours after the first intratumoral injection and analyzed for expression of inflammatory cytokines using ProcataPlex (Affymetrix). The cytokine analysis revealed that levels of IFN-α, IL-6, TNF-α, GRO α (CXCL1), MIP-1 a (CCL3), MIP-1B (CCL4) and RANTES (CCL5) were elevated in the treatment groups as compared to the controls.

In a second set of experiments, mice carrying MC38 colon carcinoma tumors (5×10⁵ cells implanted subcutaneously; tumors ˜ 100-120 mm³ in size at time of treatment) were divided into seven treatment groups and treated intratumorally with the following mRNA constructs weekly for 4 weeks (days 1, 8, 15, 22): (i) NT-MOD as a negative control; (ii) NT-MOD+STING; (iii) MLKL+STING; (iv) Diablo+STING; (v) RIPK3+STING; (vi) MLKL+Diablo+STING; and (vii) RIPK3+Diablo+STING. All groups were treated with anti-CTLA4 (intraperitoneally) at 5 mg/kg on day 1, and at 2.5 mg/kg on day 4 and 7.

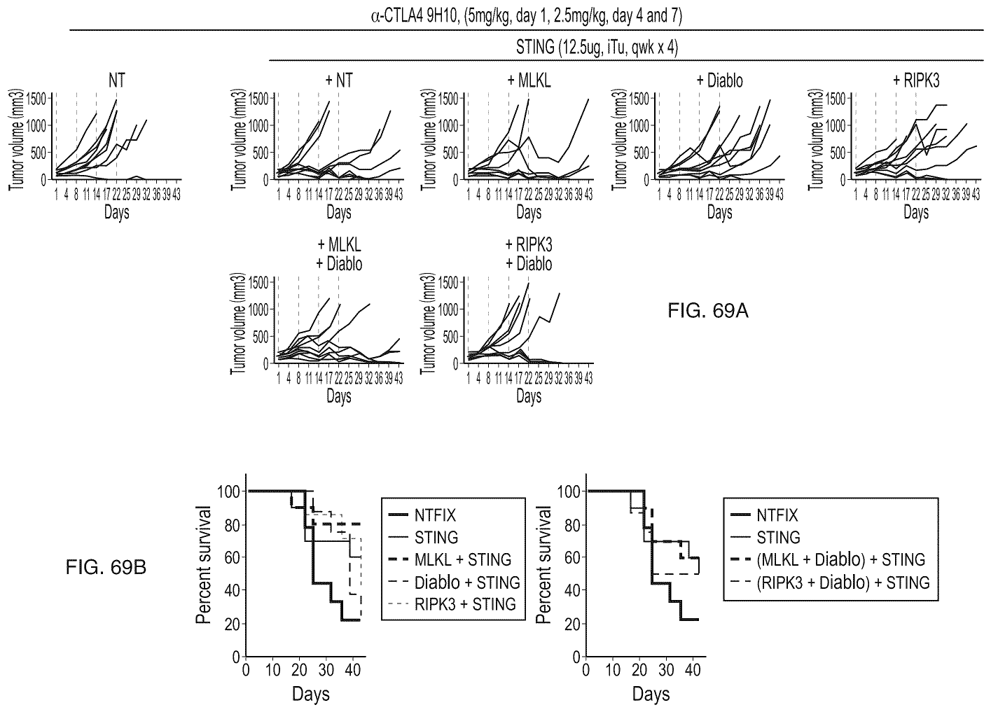

The results are shown in FIG. 69A, corresponding to the seven treatment groups described above, showing tumor volume (mm³) in the mice over the time course of the experiment, and in FIG. 69B, showing percent survival of the indicated treatment groups over the course of the experiment. Additionally, serum was collected at 10 hours and 24 hours after the first intratumoral injection and analyzed for expression of inflammatory cytokines using ProcataPlex (Affymetrix). The cytokine analysis revealed that levels of IFN-α, IL-6, TNF-α, GRO α (CXCL1), MIP-1β (CCL3), MIP-1B (CCL4) and RANTES (CCL5) were elevated in the treatment groups as compared to the controls.

In a third set of experiments, mice carrying MC38 colon carcinoma tumors (5×105 cells implanted subcutaneously) were divided into three treatment groups and treated intratumorally with the following mRNA constructs weekly for 4 weeks (days 1, 8, 15, 22): (i) vehicle control; (ii) NT-MOD+anti-PD-1; (iii) STING+anti-PD-1. Anti-PD1 was given intraperitoneally at 5 mg/kg, biweekly for 2 weeks.

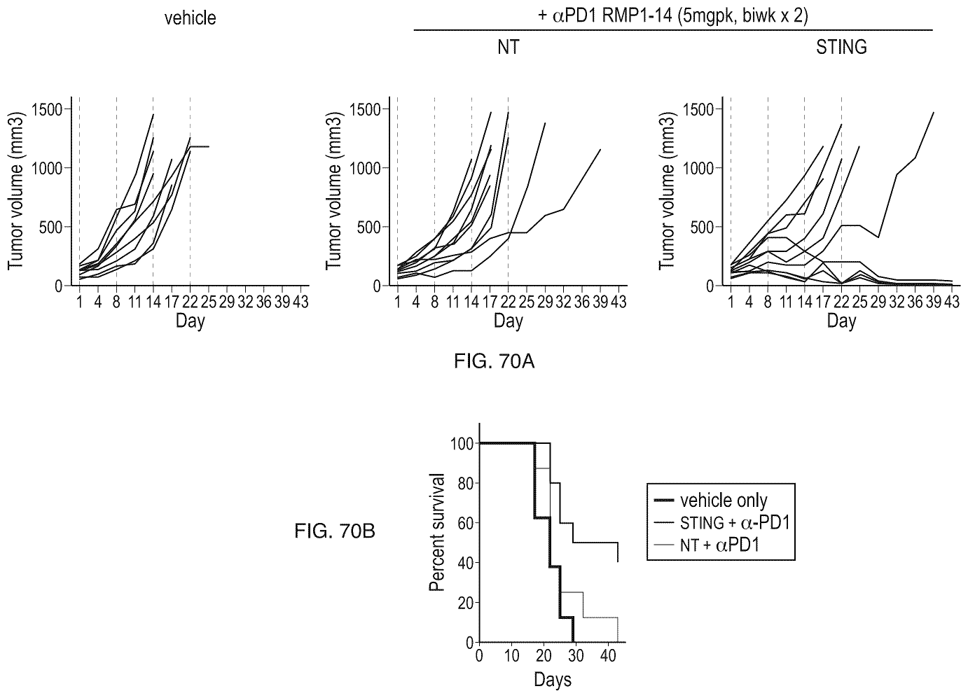

The results are shown in FIG. 70A, corresponding to the three treatment groups described above, showing tumor volume (mm³) in the mice over the time course of the experiment, and in FIG. 70B, showing percent survival of the treatment groups over the course of the experiment.

### Other Embodiments

It is to be understood that while the present disclosure has been described in conjunction with the detailed description thereof, the foregoing description is intended to illustrate and not limit the scope of the present disclosure, which is defined by the scope of the appended claims. Other aspects, advantages, and alterations are within the scope of the following claims.

All references described herein are incorporated by reference in their entireties.

SEQUENCE LISTING SUMMARY

| SEQ | |
|---|---|
| ID | |
| NO: | SEQUENCE |
| 1 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNMAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFS |
| | (huSTING(V155M); no epitope tag) |
| 2 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNVAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDtLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFS |
| | (Hu STING(R284T); no epitope tag) |
| 3 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNVAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDmLEQAKLFCRTLEDILADAPESQNNCRLIA |
| | YQEPADDSSFSLSQEVLRHLRQEEKEEVTVGSLKTSAVPSTSTMSQEPELLISGMEKPLPLRTDFS |
| | (hu STING (R284M); no epitope tag) |
| 4 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNVAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDKLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFS |
| | (Hu STING (R284K); no epitope tag) |
| 5 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFsVAHG |
| | LAWSYYIGYLRLILPELQARIRTYNQHYNNLLRGAVSQRLYILLPLDCGVPDNLSMADPNIRFLDKLPQQTGDHAGIK |
| | DRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFS |
| | (Hu STING(N154S); no epitope tag) |
| 6 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAICEKGNFNVAHG |
| | LAWSYYIGYLRLILPELQARIRTYNQHYNNLLRGAVSQRLYILLPLDCGVPDNLSMADPNIRFLDKLPQQTGDHAGIK |
| | DRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFS |
| | (Hu STING(V147L); no epitope tag) |
| 7 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNVAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIAY |
| | QqPADDSSFSLSQEVLRHLRQEEKEEVTVGSLKTSAVPSTSTMSQEPELLISGMEKPLPLRTDFS |
| | (Hu STING (E315Q); no epitope tag) |
| 8 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNVAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLaTDFS |
| | (Hu STING (R375A); no epitope tag) |
| 9 | MPHSSLHPSIPCPRGHGAQKAALVLLSACLVTLWGLGEPPEHTLRYLVLHLASLQLGLLLNGVCSLAEELR |
| | HIHSRYRGSYWRTVRACLGCPLRRGALLLLSIYFYYSLPNAVGPPFTWMLALLGLSQALNILLGLKGLAPAE |
| | ISALCEKGNFSMAHGLAWSYYIGYLRLILPELQARIRTYNQHYNNLLRGAVSQRLYILLPLDCGVPDNLSM |
| | ADPNIRFLDKLPQQTGDHAGIKDRVYSNSIYELLENGQRAGTCVLEYATPLQTLFAMSQYSQAGFSREDR |
| | LEQAKLFCRTLEDILADAPESQNNCRLIAYQEPADDSSFSLSQEVLRHLRQEEKEEVTVGSLKTSAVPSTST |
| | MSQEPELLISGMEKPLPLRTDFS |
| | (Hu STING(V147L/N154S/V155M); no epitope tag) |
| 10 | MPHSSLHPSIPCPRGHGAQKAALVLLSACLVTLWGLGEPPEHTLRYLVLHLASLQLGLLLNGVCSLAEELR |
| | HIHSRYRGSYWRTVRACLGCPLRRGALLLLSIYFYYSLPNAVGPPFTWMLALLGLSQALNILLGLKGLAPAE |
| | ISALCEKGNFSMAHGLAWSYYIGYLRLILPELQARIRTYNQHYNNLLRGAVSQRLYILLPLDCGVPDNLSM |
| | ADPNIRFLDKLPQQTGDHAGIKDRVYSNSIYELLENGQRAGTCVLEYATPLQTLFAMSQYSQAGFSRED |
| | MLEQAKLFCRTLEDILADAPESQNNCRLIAYQEPADDSSFSLSQEVLRHLRQEEKEEVTVGSLKTSAVPSTS |
| | TMSQEPELLISGMEKPLPLRTDFS |
| | (Hu STING(R284M/V147L/N154S/V155M); no epitope tag) |
| 11 | METPKPRILPWLVSQLDLGQLEGVAWLDESRTRFRIPWKHGLRQDAQMADFGIFQAWAEASGAYTPGKDKPDVS |
| | TWKRNFRSALNRKEVLRLAADNSKDPYDPHKVYEFVTPGARDFVHLGASPDTNGKSSLPHSQENLPKLFDGLILGPL |
| | KDEGSSDLAIVSDPSQQLPSPNVNNFLNPAPQENPLKQLLAEEQWEFEVTAFYRGRQVFQQTLFCPGGLRLVGSTA |
| | DMTLPWQPVTLPDPEGFLTDKLVKEYVGQVLKGLGNGLALWQAGQCLWAQRLGHSHAFWALGEELLPDSGRGP |
| | DGEVHKDKDGAVFDLRPFVADLIAFMEGSGHSPRYTLWFCMGEMWPQDQPWVKRLVMVKVVPTCLKELLEMA |
| | REGGASSLKTVDLHIDNSQPISLTSDQYKAYLQDLVEDMDFQATGNI |
| | (super mouse IRF3 S396D; no epitope tag) |
| 12 | MGTPKPRILPWLVSQLDLGQLEGVAWVNKSRTRFRIPWKHGLRQDAQQEDFGIFQAWAEATGAYVPGRDKPDLP |
| | TWKRNFRSALNRKEGLRLAEDRSKDPHDPHKIYEFVNSGVGDFSQPDTSPDTNGGGSTSDTQEDILDELLGNMVLA |
| | PLPDPGPPSLAVAPEPCPQPLRSPSLDNPTPFPNLGPSENPLKRLLVPGEEWEFEVTAFYRGRQVFQQTISCPEGLRL |
| | VGSEVGDRTLPGWPVTLPDPGMSLTDRGVMSYVRHVLSCLGGGLALWRAGQWLWAQRLGHCHTYWAVSEELL |
| | PNSGHGPDGEVPKDKEGGVFDLGPFIVDLITFTEGSGRSPRYALWFCVGESWPQDQPWTKRLVMVKVVPTCLRAL |
| | VEMARVGGASSLENTVDLHIDNSHPLSLTSDQYKAYLQDLVEGMDFQGPGET |
| | (super human IRF3 S396D; no epitope tag) |
| 13 | MALAPERAAPRVLFGEWLLGEISSGCYEGLQWLDEARTCFRVPWKHFARKDLSEADARIFKAWAVARGRWPPSSR |
| | GGGPPPEAETAERAGWKTNFRCALRSTRRFVMLRDNSGDPADPHKVYALSRELCWREGPGTDQTEAEAPAAVPP |
| | PQGGPPGPFLAHTHAGLQAPGPLPAPAGDKGDLLLQAVQQSCLADHLLTASWGADPVPTKAPGEGQEGLPLTGA |
| | CAGGPGLPAGELYGWAVETTPSPgpqpaalttgeaaapesphqaepylspspsactavqepspgaldvtimykgrtvlqkvvghps |
| | ctflygppdpavratdpqqvafpspaelpdqkqlryteellrhvapglhlelrgpqlwarrmgkckvywevggppgsaspstpacl |
| | lprncdtpifdfrvffqelvefrarqrrgsprytiylgfgqdlsagrpkekslvlvklepwlcrvhlegtqrEGVSSLDSSSLSLC |
| | LSSANSLYDDIECFLMELEQPA |
| | (Wild-type Hu IRF7 isoform A; P037 without epitope tag) |
| 14 | MALAPERAAPRVLFGEWLLGEISSGCYEGLQWLDEARTCFRVPWKHFARKDLSEADARIFKAWAVARGRWPPSSR |
| | GGGPPPEAETAERAGWKTNFRCALRSTRRFVMLRDNSGDPADPHKVYALSRELCWREGPGTDQTEAEAPAAVPP |
| | PQGGPPGPFLAHTHAGLQAPGPLPAPAGDKGDLLLQAVQQSCLADHLLTASWGADPVPTKAPGEGQEGLPLTGA |
| | CAGGPGLPAGELYGWAVETTPSPgpqpaalttgeaaapesphqaepylspspsactavqepspgaldvtimykgrtvlqkvvghps |
| | ctflygppdpavratdpqqvafpspaelpdqkqlryteellrhvapglhlelrgpqlwarrmgkckvywevggppgsaspstpacl |
| | lprncdtpifdfrvffqelvefrarqrrgsprytiylgfgqdlsagrpkekslvlvklepwlcrvhlegtqrEGVSSLDSSdLdLC |
| | LSSANSLYDDIECFLMELEQPA |
| | (constitutively active Hu IRF7 S477D/S479D; P033 without epitope tag) |
| 15 | MALAPERAAPRVLFGEWLLGEISSGCYEGLQWLDEARTCFRVPWKHFARKDLSEADARIFKAWAVARGRWPPSSR |
| | GGGPPPEAETAERAGWKTNFRCALRSTRRFVMLRDNSGDPADPHKVYALSRELCWREGPGTDQTEAEAPAAVPP |
| | PQGGPPGPFLAHTHAGLQAPGPLPAPAGDKGDLLLQAVQQSCLADHLLTASWGADPVPTKAPGEGQEGLPLTGA |
| | CAGGPGLPAGELYGWAVETTPSPgpqpaalttgeaaapesphqaepylspspsactavqepspgaldvtimykgrtvlqkvvghps |
| | ctflygppdpavratdpqqvafpspaelpdqkqlryteellrhvapglhlelrgpqlwarrmgkckvywevggppgsaspstpacl |
| | lprncdtpifdfrvffqelvefrarqrrgsprytiylgfgqdlsagrpkekslvlvklepwlcrvhlegtqrEGVSSLDdSdLSdC |
| | LSSANSLYDDIECFLMELEQPA |
| | (constitutively active Hu IRF7 S475D/S477D/L480D; P034 without epitope tag) |
| 16 | MALAPERAAPRVLFGEWLLGEISSGCYEGLQWLDEARTCFRVPWKHFARKDLSEADARIFKAWAVARGRWPPSSR |
| | GGGPPPEAETAERAGWKTNFRCALRSTRRFVMLRDNSGDPADPHKVYALSRELCWREGPGTDQTEAEAPAAVPP |
| | PQGGPPGPFLAHTHAGLQAPGPLPAPAGDKGDLLLQAVQQSCLADHLLTASWGADPVPTKAPGEGQEGLPLTGA |
| | CAGGPGLPAGELYGWAVETTPSPgpqpaalttgeaaapesphqaepylspspsactavqepspgaldvtimykgrtvlqkvvghps |
| | ctflygppdpavratdpqqvafpspaelpdqkqlryteellrhvapglhlelrgpqlwarrmgkckvywevggppgsaspstpacl |
| | lprncdtpifdfrvffqelvefrarqrrgsprytiylgfgqdlsagrpkekslvlvklepwlcrvhlegtqrEGVSSLDdddLdLC |
| | LdSANdLYDDIECFLMELEQPA |
| | (constitutively active Hu IRF7 S475D/S476D/S477D/S479D/S483D/S487D; P035 without |
| | epitope tag) |
| 17 | MALAPERAAPRVLFGEWLLGEISSGCYEGLQWLDEARTCFRVPWKHFARKDLSEADARIFKAWAVARGRWPPSSR |
| | GGGPPPEAETAERAGWKTNFRCALRSTRRFVMLRDNSGDPADPHKVYALSRELCWREGPGTDQTEAEAPAAVPP |
| | PQGGPPGPFLAHTHAGLQAPGPLPAPAGDKGDLLLQAVQQSCLADHLLTASWGADPVPTKAPGEGQEGLPLTGA |
| | CAGGPGLPAGELYGWAVETTPSPEGVSSLDSSSLSLCLSSANSLYDDIECFLMELEQPA |
| | (constitutively active truncated Hu IRF7 1-246 + 468-503; P032 without epitope tag) |
| 18 | MALAPERAAPRVLFGEWLLGEISSGCYEGLQWLDEARTCFRVPWKHFARKDLSEADARIFKAWAVARGRWPPSSR |
| | GGGPPPEAETAERAGWKTNFRCALRSTRRFVMLRDNSGDPADPHKVYALSRELCWREGPGTDQTEAEAPAAVPP |
| | PQGGPPGPFLAHTHAGLQAPGPLPAPAGDKGDLLLQAVQQSCLADHLLTASWGADPVPTKAPGEGQEGLPLTGA |
| | CAGGPGLPAGELYGWAVETTPSPEGVSSLDdddLdLCLdSANdLYDDIECFLMELEQPA |
| | (constitutively active truncated Hu IRF7 1-246 + 468-503 plus |
| | S475D/S476D/S477D/S479D/S483D/S487D; P036 without epitope tag) |
| 19 | MALAPERAAPRVLFGEWLLGEISSGCYEGLQWLDEARTCFRVPWKHFARKDLSEADARIFKAWAVARGRWPPSSR |
| | GGGPPPEAETAERAGWKTNFRCALRSTRRFVMLRDNSGDPADPHKVYALSRELCWREGPGTDQTEAEAPAAVPP |
| | PQgpqpaalttgeaaapesphqaepylspspsactavqepspgaldvtimykgrtvlqkvvghpsctflygppdpavratdpqqva |
| | fpspaelpdqkqlryteellrhvapglhlelrgpqlwarrmgkckvywevggppgsaspstpacllprncdtpifdfrvffqelve |
| | frarqrrgsprytiylgfgqdlsagrpkekslvlvklepwlcrvhlegtqrEGVSSLDSSSLSLCLSSANSLYDDIECFLMELEQPA |
| | (truncated Hu IRF7 1-151 + 247-503; P038 without epitope tag; null mutation) |
| 20 | MGGPPGPFLAHTHAGLQAPGPLPAPAGDKGDLLLQAVQQSCLADHLLTASWGADPVPTKAPGEGQEGLPLTGAC |
| | AGGPGLPAGELYGWAVETTPSPgpqpaalttgeaaapesphqaepylspspsactavqepspgaldvtimykgrtvlqkvvghpsct |
| | flygppdpavratdpqqvafpspaelpdqkqlryteellrhvapglhlelrgpqlwarrmgkckvywevggppgsaspstpacllp |
| | rncdtpifdfrvffqelvefrarqrrgsprytiylgfgqdlsagrpkekslvlvklepwlcrvhlegtqrEGVSSLDSSSLSLCLS |
| | SANSLYDDIECFLMELEQPA |
| | (truncated Hu IRF7 152-503; P039 without epitope tag; null mutation) |
| 21 | TCAAGCTTTTGGACCCTCGTACAGAAGCTAATACGACTCACTATAGGGAAATAAGAGAGAAAAGAAGAGTAAG |
| | AAGAAATATAAGAGCCACC |
| | (5′ UTR) |
| 22 | TGATAATAGGCTGGAGCCTCGGTGGCCATGCTTCTTGCCCCTTGGGCCTCCCCCCAGCCCCTCCTCCCCTTCCTG |
| | CACCCGTACCCCCGTGGTCTTTGAATAAAGTCTGAGTGGGCGGC |
| | (3′ UTR) |
| 23 | TGATAATAGGCTGGAGCCTCGGTGGCCATGCTTCTTGCCCCTTGGGCCCAAACACCATTGTCACACTCCATCCCC |
| | CCAGCCCCTCCTCCCCTTCCTCCATAAAGTAGGAAACACTACATGCACCCGTACCCCCGTGGTCTTTGAATAAAG |
| | TCTGAGTGGGCGGC |
| | (3′ UTR with miR-122 and miR-142-3p sites) |
| 24 | GSGATNFSLLKQAGDVEENPGP |
| | (2A peptide amino acid sequence) |
| 25 | GGAAGCGGAGCTACTAACTTCAGCCTGCTGAAGCAGGCTGGAGACGTGGAGGAGAACCCTGGACCT |
| | (Nucleotide sequence encoding 2A peptide) |
| 26 | TCCGGACTCAGATCCGGGGATCTCAAAATTGTCGCTCCTGTCAAACAAACTCTTAACTTTGATTTACTCAAACTG |
| | GCTGGGGATGTAGAAAGCAATCCAGGTCCACTC |
| | (Nucleotide sequence encoding 2A peptide) |
| 27 | GACAGUGCAGUCACCCAUAAAGUAGAAAGCACUACUAACAGCACUGGAGGGUGUAGUGUUUCCUACUUU |
| | AUGGAUGAGUGUACUGUG |
| | (miR-142) |
| 28 | UGUAGUGUUUCCUACUUUAUGGA |
| | (miR-142-3p) |
| 29 | UCCAUAAAGUAGGAAACACUACA |
| | (miR-142-3p binding site) |
| 30 | CAUAAAGUAGAAAGCACUACU |
| | (miR-142-5p) |
| 31 | AGUAGUGCUUUCUACUUUAUG |
| | (miR-142-5p binding site) |
| 32 | AACGCCAUUAUCACACUAAAUA |
| | (miR-122-3p) |
| 33 | UGGAGUGUGACAAUGGUGUUUG |
| | (miR-122-5p) |
| 34 | UAGCUUAUCAGACUGAUGUUGA |
| | (miR-21-5p) |
| 35 | CAACACCAGUCGAUGGGCUGU |
| | (miR-21-3p) |
| 36 | MHQKRTAMFQDPQER |
| | (HPV E6 peptide) |
| 37 | RTAMFQDPQERPRKL |
| | (HPV E6 peptide) |
| 38 | FQDPQERPRKLPQLC |
| | (HPV E6 peptide) |
| 39 | QERPRKLPQLCTELQ |
| | (HPV E6 peptide) |
| 40 | RKLPQLCTELQTTIH |
| | (HPV E6 peptide) |
| 41 | QLCTELQTTIHDIIL |
| | (HPV E6 peptide) |
| 42 | ELQTTIHDIILECVY |
| | (HPV E6 peptide) |
| 43 | LECVYCKQQLLRRII |
| | (HPV E6 peptide) |
| 44 | IILECVYCKQQLLRR |
| | (HPV E6 peptide) |
| 45 | CVYCKQQLLRREVYD |
| 46 | KQQLLRREVYDFAFR |
| | (HPV E6 peptide) |
| 47 | LRREVYDFAFRDLCI |
| | (HPV E6 peptide) |
| 48 | VYDFAFRDLCIVYRD |
| | (HPV E6 peptide) |
| 49 | AFRDLCIVYRDGNPY |
| | (HPV E6 peptide) |
| 50 | LCIVYRDGNPYAVCD |
| | (HPV E6 peptide) |
| 51 | YRDGNPYAVCDKCLK |
| | (HPV E6 peptide) |
| 52 | NPYAVCDKCLKFYSK |
| | (HPV E6 peptide) |
| 53 | VCDKCLKFYSKISEY |
| | (HPV E6 peptide) |
| 54 | CLKFYSKISEYRHYC |
| | (HPV E6 peptide) |
| 55 | YSKISEYRHYCYSLY |
| | (HPV E6 peptide) |
| 56 | SEYRHYCYSLYGTTL |
| | (HPV E6 peptide) |
| 57 | HYCYSLYGTTLEQQY |
| | (HPV E6 peptide) |
| 58 | SLYGTTLEQQYNKPL |
| | (HPV E6 peptide) |
| 59 | TTLEQQYNKPLCDLL |
| | (HPV E6 peptide) |
| 60 | QQYNKPLCDLLIRCI |
| | (HPV E6 peptide) |
| 61 | KPLCDLLIRCINCQK |
| | (HPV E6 peptide) |
| 62 | DLLIRCINCQKPLCP |
| | (HPV E6 peptide) |
| 63 | RCINCQKPLCPEEKQ |
| | (HPV E6 peptide) |
| 64 | CQKPLCPEEKQRHLD |
| | (HPV E6 peptide) |
| 65 | LCPEEKQRHLDKKQR |
| | (HPV E6 peptide) |
| 66 | EKQRHLDKKQRFHNI |
| | (HPV E6 peptide) |
| 67 | HLDKKQRFHNIRGRW |
| | (HPV E6 peptide) |
| 68 | KQRFHNIRGRWTGRC |
| | (HPV E6 peptide) |
| 69 | HNIRGRWTGRCMSCC |
| | (HPV E6 peptide) |
| 70 | GRWTGRCMSCCRSSR |
| | (HPV E6 peptide) |
| 71 | GRCMSCCRSSRTRRE |
| | (HPV E6 peptide) |
| 72 | SCCRSSRTRRETQL |
| | (HPV E6 peptide) |
| 73 | MHGDTPTLHEYMLDL |
| | (HPV E7 peptide) |
| 74 | TPTLHEYMLDLQPET |
| | (HPV E7 peptide) |
| 75 | HEYMLDLQPETTDLY |
| | (HPV E7 peptide) |
| 76 | LDLQPETTDLYCYEQ |
| | (HPV E7 peptide) |
| 77 | PETTDLYCYEQLNDS |
| | (HPV E7 peptide) |
| 78 | DLYCYEQLNDSSEEE |
| | (HPV E7 peptide) |
| 79 | YEQLNDSSEEEDEID |
| | (HPV E7 peptide) |
| 80 | NDSSEEEDEIDGPAG |
| | (HPV E7 peptide) |
| 81 | EEEDEIDGPAGQAEP |
| | (HPV E7 peptide) |
| 82 | EIDGPAGQAEPDRAH |
| | (HPV E7 peptide) |
| 83 | PAGQAEPDRAHYNIV |
| | (HPV E7 peptide) |
| 84 | AEPDRAHYNIVTFCC |
| | (HPV E7 peptide) |
| 85 | RAHYNIVTFCCKCDS |
| | (HPV E7 peptide) |
| 86 | NIVTFCCKCDSTLRL |
| | (HPV E7 peptide) |
| 87 | FCCKCDSTLRLCVQS |
| | (HPV E7 peptide) |
| 88 | CDSTLRLCVQSTHVD |
| | (HPV E7 peptide) |
| 89 | LRLCVQSTHVDIRTL |
| | (HPV E7 peptide) |
| 90 | VQSTHVDIRTLEDLL |
| | (HPV E7 peptide) |
| 91 | HVDIRTLEDLLMGTL |
| | (HPV E7 peptide) |
| 92 | RTLEDLLMGTLGIVC |
| | (HPV E7 peptide) |
| 93 | DLLMGTLGIVCPICS |
| | (HPV E7 peptide) |
| 94 | GTLGIVCPICSQKP |
| | (HPV E7 peptide) |
| 95 | MKLVVVGADGVGKSAL |
| | (KRAS(G12D)15 mer) |
| 96 | MKLVVVGAVGVGKSAL |
| | (KRAS(G12V)15 mer) |
| 97 | MLVVVGAGDVGKSALT |
| | (KRAS(G13D)15 mer) |
| 98 | MTEYKLVVVGADGVGKSALTIQLIQ |
| | (KRAS(G12D)25 mer) |
| 99 | MTEYKLVVVGAVGVGKSALTIQLIQ |
| | (KRAS(G12V)25 mer) |
| 100 | MTEYKLVVVGAGDVGKSALTIQLIQ |
| | (KRAS(G13D)25 mer) |
| 101 | MKLVVVGADGVGKSALKLVVVGADGVGKSALKLVVVGADGVGKSAL |
| | (KRAS(G12D)15 mer^{∧}3) |
| 102 | MKLVVVGAVGVGKSALKLVVVGAVGVGKSALKLVVVGAVGVGKSAL |
| | (KRAS(G12V)15 mer^{∧}3) |
| 103 | MLVVVGAGDVGKSALTLVVVGAGDVGKSALTLVVVGAGDVGKSALT |
| | (KRAS(G13D)15 mer^{∧}3) |
| 104 | MTEYKLVVVGADGVGKSALTIQLIQMTEYKLVVVGADGVGKSALTIQLIQMTEYKLVVVGADGVGKSALTIQLIQ |
| | (KRAS(G12D)25 mer^{∧}3) |
| 105 | MTEYKLVVVGAVGVGKSALTIQLIQMTEYKLVVVGAVGVGKSALTIQLIQMTEYKLVVVGAVGVGKSALTIQLIQ |
| | (KRAS(G12V)25 mer^{∧}3) |
| 106 | MTEYKLVVVGAGDVGKSALTIQLIQMTEYKLVVVGAGDVGKSALTIQLIQMTEYKLVVVGAGDVGKSALTIQLIQ |
| | (KRAS(G13D)25 mer^{∧}3) |
| 107 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNMAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIA |
| | YQEPADDSSFSLSQEVLRHLRQEEKEEVTVGSLKTSAVPSTSTMSQEPELLISGMEKPLPLRTDFSATNFSLLKQAGDV |
| | EENPGPMKLVVVGADGVGKSAL |
| | (KRAS(G12D)15 mer_nt.STING(V155M)) |
| 108 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNMAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIA |
| | YQEPADDSSFSLSQEVLRHLRQEEKEEVTVGSLKTSAVPSTSTMSQEPELLISGMEKPLPLRTDFSATNFSLLKQAGDV |
| | EENPGPMKLVVVGAVGVGKSAL |
| | (KRAS(G12V)15 mer_nt.STING(V155M) |
| 109 | MPHSSLHPSIPCPRGHGAQKAALVLLSACLVTLWGLGEPPEHTLRYLVLHLASLQLGLLLNGVCSLAEELRHIHSRYR |
| | GSYWRTVRACLGCPLRRGALLLLSIYFYYSLPNAVGPPFTWMLALLGLSQALNILLGLKGLAPAEISAVCEKGNFNM |
| | AHGLAWSYYIGYLRLILPELQARIRTYNQHYNNLLRGAVSQRLYILLPLDCGVPDNLSMADPNIRFLDKLPQQTGDH |
| | AGIKDRVYSNSIYELLENGQRAGTCVLEYATPLQTLFAMSQYSQAGFSREDRLEQAKLFCRTLEDILADAPESQNNC |
| | RLIAYQEPADDSSFSLSQEVLRHLRQEEKEEVTVGSLKTSAVPSTSTMSQEPELLISGMEKPLPLRTDFSATNFSLLKQ |
| | AGDVEENPGPMLVVVGAGDVGKSALT |
| | (KRAS(G13D)15 mer_nt.STING(V155M) |
| 110 | MPHSSLHPSIPCPRGHGAQKAALVLLSACLVTLWGLGEPPEHTLRYLVLHLASLQLGLLLNGVCSLAEELRHIHSRYR |
| | GSYWRTVRACLGCPLRRGALLLLSIYFYYSLPNAVGPPFTWMLALLGLSQALNILLGLKGLAPAEISAVCEKGNFNMA |
| | HGLAWSYYIGYLRLILPELQARIRTYNQHYNNLLRGAVSQRLYILLPLDCGVPDNLSMADPNIRFLDKLPQQTGDHA |
| | GIKDRVYSNSIYELLENGQRAGTCVLEYATPLQTLFAMSQYSQAGFSREDRLEQAKLFCRTLEDILADAPESQNNCRLI |
| | AYQEPADDSSFSLSQEVLRHLRQEEKEEVTVGSLKTSAVPSTSTMSQEPELLISGMEKPLPLRTDFSATNFSLLKQAGD |
| | VEENPGPMTEYKLVVVGADGVGKSALTIQLIQ |
| | (KRAS(G12D)25 mer_nt.STING(V155M)) |
| 111 | MPHSSLHPSIPCPRGHGAQKAALVLLSACLVTLWGLGEPPEHTLRYLVLHLASLQLGLLLNGVCSLAEELRHIHSRYR |
| | GSYWRTVRACLGCPLRRGALLLLSIYFYYSLPNAVGPPFTWMLALLGLSQALNILLGLKGLAPAEISAVCEKGNFNMA |
| | HGLAWSYYIGYLRLILPELQARIRTYNQHYNNLLRGAVSQRLYILLPLDCGVPDNLSMADPNIRFLDKLPQQTGDHA |
| | GIKDRVYSNSIYELLENGQRAGTCVLEYATPLQTLFAMSQYSQAGFSREDRLEQAKLFCRTLEDILADAPESQNNCRLI |
| | AYQEPADDSSFSLSQEVLRHLRQEEKEEVTVGSLKTSAVPSTSTMSQEPELLISGMEKPLPLRTDFSATNFSLLKQAGD |
| | VEENPGPMTEYKLVVVGAVGVGKSALTIQLIQ |
| | (KRAS(G12V)25 mer_nt.STING(V155M) |
| 112 | MPHSSLHPSIPCPRGHGAQKAALVLLSACLVTLWGLGEPPEHTLRYLVLHLASLQLGLLLNGVCSLAEELRHIHSRYR |
| | GSYWRTVRACLGCPLRRGALLLLSIYFYYSLPNAVGPPFTWMLALLGLSQALNILLGLKGLAPAEISAVCEKGNFNMA |
| | HGLAWSYYIGYLRLILPELQARIRTYNQHYNNLLRGAVSQRLYILLPLDCGVPDNLSMADPNIRFLDKLPQQTGDHA |
| | GIKDRVYSNSIYELLENGQRAGTCVLEYATPLQTLFAMSQYSQAGFSREDRLEQAKLFCRTLEDILADAPESQNNCRLI |
| | AYQEPADDSSFSLSQEVLRHLRQEEKEEVTVGSLKTSAVPSTSTMSQEPELLISGMEKPLPLRTDFSATNFSLLKQAGD |
| | VEENPGPMTEYKLVVVGAGDVGKSALTIQLIQ |
| | (KRAS(G13D)25 mer_nt.STING(V155M) |
| 113 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNMAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFSATNFSLLKQAGDVE |
| | ENPGPMKLVVVGADGVGKSALKLVVVGADGVGKSALKLVVVGADGVGKSAL |
| | (KRAS(G12D)15 mer^{∧}3_nt.STING(V155M)) |
| 114 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNMAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFSATNFSLLKQAGDVE |
| | ENPGPMKLVVVGAVGVGKSALKLVVVGAVGVGKSALKLVVVGAVGVGKSAL |
| | (KRAS(G12V)15 mer^{∧}3_nt.STING(V155M) |
| 115 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNMAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFSATNFSLLKQAGDVE |
| | ENPGPMLVVVGAGDVGKSALTLVVVGAGDVGKSALTLVVVGAGDVGKSALT |
| | (KRAS(G13D)15 mer^{∧}3_nt.STING(V155M) |
| 116 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNMAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFSATNFSLLKQAGDVE |
| | ENPGPMTEYKLVVVGADGVGKSALTIQLIQMTEYKLVVVGADGVGKSALTIQLIQMTEYKLVVVGADGVGKSALTI |
| | QLIQ |
| | (KRAS(G12D)25 mer^{∧}3_nt.STING(V155M)) |
| 117 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNMAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFSATNFSLLKQAGDVE |
| | ENPGPMTEYKLVVVGAVGVGKSALTIQLIQMTEYKLVVVGAVGVGKSALTIQLIQMTEYKLVVVGAVGVGKSALTIQ |
| | LIQ |
| | (KRAS(G12V)25 mer^{∧}3_nt.STING(V155M) |
| 118 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNMAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDRLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFSATNFSLLKQAGDVE |
| | ENPGPMTEYKLVVVGAGDVGKSALTIQLIQMTEYKLVVVGAGDVGKSALTIQLIQMTEYKLVVVGAGDVGKSALTI |
| | QLIQ |
| | (KRAS(G13D)25 mer^{∧}3_nt.STING(V155M) |
| 119 | MKLVVVGADGVGKSALATNFSLLKQAGDVEENPGPMPHSSLHPSIPCPRGHGAQKAALVLLSACLVTLWGLGEPP |
| | EHTLRYLVLHLASLQLGLLLNGVCSLAEELRHIHSRYRGSYWRTVRACLGCPLRRGALLLLSIYFYYSLPNAVGPPFTW |
| | MLALLGLSQALNILLGLKGLAPAEISAVCEKGNFNMAHGLAWSYYIGYLRLILPELQARIRTYNQHYNNLLRGAVSQR |
| | LYILLPLDCGVPDNLSMADPNIRFLDKLPQQTGDHAGIKDRVYSNSIYELLENGQRAGTCVLEYATPLQTLFAMSQYS |
| | QAGFSREDRLEQAKLFCRTLEDILADAPESQNNCRLIAYQEPADDSSFSLSQEVLRHLRQEEKEEVTVGSLKTSAVPST |
| | STMSQEPELLISGMEKPLPLRTDFS |
| | (KRAS(G12D)15 mer_ct.STING(V155M)) |
| 120 | MKLVVVGAVGVGKSALATNFSLLKQAGDVEENPGPMPHSSLHPSIPCPRGHGAQKAALVLLSACLVTLWGLGEPP |
| | EHTLRYLVLHLASLQLGLLLNGVCSLAEELRHIHSRYRGSYWRTVRACLGCPLRRGALLLLSIYFYYSLPNAVGPPFTW |
| | MLALLGLSQALNILLGLKGLAPAEISAVCEKGNFNMAHGLAWSYYIGYLRLILPELQARIRTYNQHYNNLLRGAVSQR |
| | LYILLPLDCGVPDNLSMADPNIRFLDKLPQQTGDHAGIKDRVYSNSIYELLENGQRAGTCVLEYATPLQTLFAMSQYS |
| | QAGFSREDRLEQAKLFCRTLEDILADAPESQNNCRLIAYQEPADDSSFSLSQEVLRHLRQEEKEEVTVGSLKTSAVPST |
| | STMSQEPELLISGMEKPLPLRTDFS |
| | (KRAS(G12V)15 mer_ct.STING(V155M) |
| 121 | MLVVVGAGDVGKSALTATNFSLLKQAGDVEENPGPMPHSSLHPSIPCPRGHGAQKAALVLLSACLVTLWGLGEPP |
| | EHTLRYLVLHLASLQLGLLLNGVCSLAEELRHIHSRYRGSYWRTVRACLGCPLRRGALLLLSIYFYYSLPNAVGPPFTW |
| | MLALLGLSQALNILLGLKGLAPAEISAVCEKGNFNMAHGLAWSYYIGYLRLILPELQARIRTYNQHYNNLLRGAVSQR |
| | LYILLPLDCGVPDNLSMADPNIRFLDKLPQQTGDHAGIKDRVYSNSIYELLENGQRAGTCVLEYATPLQTLFAMSQYS |
| | QAGFSREDRLEQAKLFCRTLEDILADAPESQNNCRLIAYQEPADDSSFSLSQEVLRHLRQEEKEEVTVGSLKTSAVPST |
| | STMSQEPELLISGMEKPLPLRTDFS |
| | (KRAS(G13D)15 mer_ct.STING(V155M) |
| 122 | MTEYKLVVVGADGVGKSALTIQLIQATNFSLLKQAGDVEENPGPMPHSSLHPSIPCPRGHGAQKAALVLLSACLVTL |
| | WGLGEPPEHTLRYLVLHLASLQLGLLLNGVCSLAEELRHIHSRYRGSYWRTVRACLGCPLRRGALLLLSIYFYYSLPNAV |
| | GPPFTWMLALLGLSQALNILLGLKGLAPAEISAVCEKGNFNMAHGLAWSYYIGYLRLILPELQARIRTYNQHYNNLLR |
| | GAVSQRLYILLPLDCGVPDNLSMADPNIRFLDKLPQQTGDHAGIKDRVYSNSIYELLENGQRAGTCVLEYATPLQTLF |
| | AMSQYSQAGFSREDRLEQAKLFCRTLEDILADAPESQNNCRLIAYQEPADDSSFSLSQEVLRHLRQEEKEEVTVGSLK |
| | TSAVPSTSTMSQEPELLISGMEKPLPLRTDFS |
| | (KRAS(G12D)25 mer_ct.STING(V155M)) |
| 123 | MTEYKLVVVGAVGVGKSALTIQLIQATNFSLLKQAGDVEENPGPMPHSSLHPSIPCPRGHGAQKAALVLLSACLVTL |
| | WGLGEPPEHTLRYLVLHLASLQLGLLLNGVCSLAEELRHIHSRYRGSYWRTVRACLGCPLRRGALLLLSIYFYYSLPNAV |
| | GPPFTWMLALLGLSQALNILLGLKGLAPAEISAVCEKGNFNMAHGLAWSYYIGYLRLILPELQARIRTYNQHYNNLLR |
| | GAVSQRLYILLPLDCGVPDNLSMADPNIRFLDKLPQQTGDHAGIKDRVYSNSIYELLENGQRAGTCVLEYATPLQTLF |
| | AMSQYSQAGFSREDRLEQAKLFCRTLEDILADAPESQNNCRLIAYQEPADDSSFSLSQEVLRHLRQEEKEEVTVGSLK |
| | TSAVPSTSTMSQEPELLISGMEKPLPLRTDFS |
| | (KRAS(G12V)25 mer_ct.STING(V155M) |
| 124 | MTEYKLVVVGAGDVGKSALTIQLIQATNFSLLKQAGDVEENPGPMPHSSLHPSIPCPRGHGAQKAALVLLSACLVTL |
| | WGLGEPPEHTLRYLVLHLASLQLGLLLNGVCSLAEELRHIHSRYRGSYWRTVRACLGCPLRRGALLLLSIYFYYSLPNAV |
| | GPPFTWMLALLGLSQALNILLGLKGLAPAEISAVCEKGNFNMAHGLAWSYYIGYLRLILPELQARIRTYNQHYNNLLR |
| | GAVSQRLYILLPLDCGVPDNLSMADPNIRFLDKLPQQTGDHAGIKDRVYSNSIYELLENGQRAGTCVLEYATPLQTLF |
| | AMSQYSQAGFSREDRLEQAKLFCRTLEDILADAPESQNNCRLIAYQEPADDSSFSLSQEVLRHLRQEEKEEVTVGSLK |
| | TSAVPSTSTMSQEPELLISGMEKPLPLRTDFS |
| | (KRAS(G13D)25 mer_ct.STING(V155M) |
| 125 | MKLVVVGADGVGKSALKLVVVGADGVGKSALKLVVVGADGVGKSALATNFSLLKQAGDVEENPGPMPHSSLHPSI |
| | PCPRGHGAQKAALVLLSACLVTLWGLGEPPEHTLRYLVLHLASLQLGLLLNGVCSLAEELRHIHSRYRGSYWRTVRAC |
| | LGCPLRRGALLLLSIYFYYSLPNAVGPPFTWMLALLGLSQALNILLGLKGLAPAEISAVCEKGNFNMAHGLAWSYYIGY |
| | LRLILPELQARIRTYNQHYNNLLRGAVSQRLYILLPLDCGVPDNLSMADPNIRFLDKLPQQTGDHAGIKDRVYSNSIYE |
| | LLENGQRAGTCVLEYATPLQTLFAMSQYSQAGFSREDRLEQAKLFCRTLEDILADAPESQNNCRLIAYQEPADDSSFS |
| | LSQEVLRHLRQEEKEEVTVGSLKTSAVPSTSTMSQEPELLISGMEKPLPLRTDFS |
| | (KRAS(G12D)15 mer^{∧}3_ct.STING(V155M)) |
| 126 | MKLVVVGAVGVGKSALKLVVVGAVGVGKSALKLVVVGAVGVGKSALATNFSLLKQAGDVEENPGPMPHSSLHPSI |
| | PCPRGHGAQKAALVLLSACLVTLWGLGEPPEHTLRYLVLHLASLQLGLLLNGVCSLAEELRHIHSRYRGSYWRTVRAC |
| | LGCPLRRGALLLLSIYFYYSLPNAVGPPFTWMLALLGLSQALNILLGLKGLAPAEISAVCEKGNFNMAHGLAWSYYIGY |
| | LRLILPELQARIRTYNQHYNNLLRGAVSQRLYILLPLDCGVPDNLSMADPNIRFLDKLPQQTGDHAGIKDRVYSNSIYE |
| | LLENGQRAGTCVLEYATPLQTLFAMSQYSQAGFSREDRLEQAKLFCRTLEDILADAPESQNNCRLIAYQEPADDSSFS |
| | LSQEVLRHLRQEEKEEVTVGSLKTSAVPSTSTMSQEPELLISGMEKPLPLRTDFS |
| | (KRAS(G12V)15 mer^{∧}3_ct.STING(V155M) |
| 127 | MLVVVGAGDVGKSALTLVVVGAGDVGKSALTLVVVGAGDVGKSALTATNFSLLKQAGDVEENPGPMPHSSLHPSI |
| | PCPRGHGAQKAALVLLSACLVTLWGLGEPPEHTLRYLVLHLASLQLGLLLNGVCSLAEELRHIHSRYRGSYWRTVRAC |
| | LGCPLRRGALLLLSIYFYYSLPNAVGPPFTWMLALLGLSQALNILLGLKGLAPAEISAVCEKGNFNMAHGLAWSYYIGY |
| | LRLILPELQARIRTYNQHYNNLLRGAVSQRLYILLPLDCGVPDNLSMADPNIRFLDKLPQQTGDHAGIKDRVYSNSIYE |
| | LLENGQRAGTCVLEYATPLQTLFAMSQYSQAGFSREDRLEQAKLFCRTLEDILADAPESQNNCRLIAYQEPADDSSFS |
| | LSQEVLRHLRQEEKEEVTVGSLKTSAVPSTSTMSQEPELLISGMEKPLPLRTDFS |
| | (KRAS(G13D)15 mer^{∧}3_ct.STING(V155M) |
| 128 | MTEYKLVVVGADGVGKSALTIQLIQMTEYKLVVVGADGVGKSALTIQLIQMTEYKLVVVGADGVGKSALTIQLIQAT |
| | NFSLLKQAGDVEENPGPMPHSSLHPSIPCPRGHGAQKAALVLLSACLVTLWGLGEPPEHTLRYLVLHLASLQLGLLLN |
| | GVCSLAEELRHIHSRYRGSYWRTVRACLGCPLRRGALLLLSIYFYYSLPNAVGPPFTWMLALLGLSQALNILLGLKGLA |
| | PAEISAVCEKGNFNMAHGLAWSYYIGYLRLILPELQARIRTYNQHYNNLLRGAVSQRLYILLPLDCGVPDNLSMADP |
| | NIRFLDKLPQQTGDHAGIKDRVYSNSIYELLENGQRAGTCVLEYATPLQTLFAMSQYSQAGFSREDRLEQAKLFCRTL |
| | EDILADAPESQNNCRLIAYQEPADDSSFSLSQEVLRHLRQEEKEEVTVGSLKTSAVPSTSTMSQEPELLISGMEKPLPL |
| | RTDFS |
| | (KRAS(G12D)25 mer^{∧}3_ct.STING(V155M)) |
| 129 | MTEYKLVVVGAVGVGKSALTIQLIQMTEYKLVVVGAVGVGKSALTIQLIQMTEYKLVVVGAVGVGKSALTIQLIQAT |
| | NFSLLKQAGDVEENPGPMPHSSLHPSIPCPRGHGAQKAALVLLSACLVTLWGLGEPPEHTLRYLVLHLASLQLGLLLN |
| | GVCSLAEELRHIHSRYRGSYWRTVRACLGCPLRRGALLLLSIYFYYSLPNAVGPPFTWMLALLGLSQALNILLGLKGLA |
| | PAEISAVCEKGNFNMAHGLAWSYYIGYLRLILPELQARIRTYNQHYNNLLRGAVSQRLYILLPLDCGVPDNLSMADP |
| | NIRFLDKLPQQTGDHAGIKDRVYSNSIYELLENGQRAGTCVLEYATPLQTLFAMSQYSQAGFSREDRLEQAKLFCRTL |
| | EDILADAPESQNNCRLIAYQEPADDSSFSLSQEVLRHLRQEEKEEVTVGSLKTSAVPSTSTMSQEPELLISGMEKPLPL |
| | RTDFS |
| | (KRAS(G12V)25 mer^{∧}3_ct.STING(V155M) |
| 130 | MTEYKLVVVGAGDVGKSALTIQLIQMTEYKLVVVGAGDVGKSALTIQLIQMTEYKLVVVGAGDVGKSALTIQLIQAT |
| | NFSLLKQAGDVEENPGPMPHSSLHPSIPCPRGHGAQKAALVLLSACLVTLWGLGEPPEHTLRYLVLHLASLQLGLLLN |
| | GVCSLAEELRHIHSRYRGSYWRTVRACLGCPLRRGALLLLSIYFYYSLPNAVGPPFTWMLALLGLSQALNILLGLKGLA |
| | PAEISAVCEKGNFNMAHGLAWSYYIGYLRLILPELQARIRTYNQHYNNLLRGAVSQRLYILLPLDCGVPDNLSMADP |
| | NIRFLDKLPQQTGDHAGIKDRVYSNSIYELLENGQRAGTCVLEYATPLQTLFAMSQYSQAGFSREDRLEQAKLFCRTL |
| | EDILADAPESQNNCRLIAYQEPADDSSFSLSQEVLRHLRQEEKEEVTVGSLKTSAVPSTSTMSQEPELLISGMEKPLPL |
| | RTDFS |
| | (KRAS(G13D)25 mer^{∧}3_ct.STING(V155M) |
| 131 | MTEYKLVVVGACGVGKSALTIQLIQ |
| | (KRAS(G12C)25 mer) |
| 132 | MTEYKLVVVGACGVGKSALTIQLIQMTEYKLVVVGACGVGKSALTIQLIQMTEYKLVVVGACGVGKSALTIQLIQ |
| | (KRAS(G12C)25 mer^{∧}3) |
| 133 | MTEYKLVVVGAGGVGKSALTIQLIQ |
| | (KRAS(WT)25 mer) |
| 134 | MSAGDPRVGSGSLDSFMFSIPLVALNVGVRRRLSLFLNPRTPVAADWTLLAEEMGFEYLEIRELETRPDPTRSLLDA |
| | WQGRSGASVGRLLELLALLDREDILKELKSRIEEDCQKYLGKQQNQESEKPLQVARVESSVPQTKELGGITTLDDPLG |
| | QTPELFDAFICYCPNDIEFVQEMIRQLEQTDYRLKLCVSDRDVLPGTCVWSIASELIEKRCRRMVVVVSDDYLQSKEC |
| | DFQTKFALSLSPGVQQKRPIPIKYKAMKKDFPSILRFITICDYTNPCTKSWFWTRLAKALSLP |
| | (human myd88(L265P); P4027 without epitope tag) |
| 135 | MAAGGPGAGSAAPVSSTSSLPLAALNMRVRRRLSLFLNVRTQVAADWTALAEEMDFEYLEIRQLETQADPTGRLLD |
| | AWQGRPGASVGRLLELLTKLGRDDVLLELGPSIEEDCQKYILKQQQEEAEKPLQVAAVDSSVPRTAELAGITTLDDPL |
| | GHMPERFDAFICYCPSDIQFVQEMIRQLEQTNYRLKLCVSDRDVLPGTCVWSIASELIEKRCRRMVVVVSDDYLQSK |
| | ECDFQTKFALSLSPGAHQKRPIPIKYKAMKKEFPSILRFITVCDYTNPCTKSWFWTRLAKALSLP |
| | (mouse myd88(L265P); P4028 without epitope tag) |
| 136 | MGVGKSKLDKCPLSWHKKDSVDADQDGHESDSKNSEEACLRGFVEQSSGSEPPTGEQDQPEAKGAGPEEQDEEE |
| | FLKFVILHAEDDTDEALRVQDLLQNDFGIRPGIVFAEMPCGRLHLQNLDDAVNGSAWTILLLTENFLRDTWCNFQFY |
| | TSLMNSVSRQHKYNSVIPMRPLNSPLPRERTPLALQTINALEEESQGFSTQVERIFRESVFERQQSIWKETRSVSQKQ |
| | FIA |
| | (Mouse TRAM (TICAM2); P4033 without epitope tag) |
| 137 | MSLWGLVSKMPPEKVQRLYVDFPQHLRHLLGDWLESQPWEFLVGSDAFCCNLASALLSDTVQHLQASVGEQGEG |
| | STILQHISTLESIYQRDPLKLVATFRQILQGEKKAVMEQFRHLPMPFHWKQEELKFKTGLRRLQHRVGEIHLLREALQK |
| | GAEAGQVSLHSLIETPANGTGPSEALAMLLQETTGELEAAKALVLKRIQIWKRQQQLAGNGAPFEESLAPLQERCES |
| | LVDIYSQLQQEVGAAGGELEPKTRASLTGRLDEVLRTLVTSCFLVEKQPPQVLKTQTKFQAGVRFLLGLRFLGAPAKP |
| | PLVRADMVTEKQARELSVPQGPGAGAESTGEIINNTVPLENSIPGNCCSALFKNLLLKKIKRCERKGTESVTEEKCAVL |
| | FSASFTLGPGKLPIQLQALSLPLVVIVHGNQDNNAKATILWDNAFSEMDRVPFVVAERVPWEKMCETLNLKFMAEV |
| | GTNRGLLPEHFLFLAQKIFNDNSLSMEAFQHRSVSWSQFNKEILLGRGFTFWQWFDGVLDLTKRCLRSYWSDRLIIG |
| | FISKQYAASLLLNEPDGTFLLRFSDSEIGGITIAHVIRGQDGSPQIENIQPFSAKDLSIRSLGDRIRDLAQLKNLYPKKPKD |
| | EAFRSHYKPEQMGKDGRGYVPATIKMTVERDQPLPTPELQMPTMVPSYDLGMAPDSSMSMQLGPDMVPQVYP |
| | PHSHSIPPYQGLSPEESVNVLSAFQEPHLQMPPSLGQMSLPFDQPHPQGLLPCQPQEHAVSSPDPLLCSDVTMVED |
| | SCLSQPVTAFPQGTWIGEDIFPPLLPPTEQDLTKLLLEGQGESGGGSLGAQPLLQPSHYGQSGISMSHMDLRANPS |
| | W |
| | (STAT6 V547A/T548A); P008 with no epitope tag) |
| 138 | MSLWGLVSKMPPEKVQRLYVDFPQHLRHLLGDWLESQPWEFLVGSDAFCCNLASALLSDTVQHLQASVGEQGEG |
| | STILQHISTLESIYQRDPLKLVATFRQILQGEKKAVMEQFRHLPMPFHWKQEELKFKTGLRRLQHRVGEIHLLREALQK |
| | GAEAGQVSLHSLIETPANGTGPSEALAMLLQETTGELEAAKALVLKRIQIWKRQQQLAGNGAPFEESLAPLQERCES |
| | LVDIYSQLQQEVGAAGGELEPKTRASLTGRLDEVLRTLVTSCFLVEKQPPQVLKTQTKFQAGVRFLLGLRFLGAPAKP |
| | PLVRADMVTEKQARELSVPQGPGAGAESTGEIINNTVPLENSIPGNCCSALFKNLLLKKIKRCERKGTESVTEEKCAVL |
| | FSASFTLGPGKLPIQLQALDLPLVVIVHGNQDNNAKATILWDNAFSEMDRVPFVVAERVPWEKMCETLNLKFMAE |
| | VGTNRGLLPEHFLFLAQKIFNDNSLSMEAFQHRSVSWSQFNKEILLGRGFTFWQWFDGVLDLTKRCLRSYWSDRLII |
| | GFISKQYVTSLLLNEPDGTFLLRFSDSEIGGITIAHVIRGQDGSPQIENIQPFSAKDLSIRSLGDRIRDLAQLKNLYPKKPK |
| | DEAFRSHYKPEQMGKDGRGYVPATIKMTVERDQPLPTPELQMPTMVPSYDLGMAPDSSMSMQLGPDMVPQVY |
| | PPHSHSIPPYQGLSPEESVNVLSAFQEPHLQMPPSLGQMSLPFDQPHPQGLLPCQPQEHAVSSPDPLLCSDVTMVE |
| | DSCLSQPVTAFPQGTWIGEDIFPPLLPPTEQDLTKLLLEGQGESGGGSLGAQPLLQPSHYGQSGISMSHMDLRANPS |
| | W |
| | (STAT6 (S407D); P009 with no epitope tag) |
| 139 | MSLWGLVSKMPPEKVQRLYVDFPQHLRHLLGDWLESQPWEFLVGSDAFCCNLASALLSDTVQHLQASVGEQGEG |
| | STILQHISTLESIYQRDPLKLVATFRQILQGEKKAVMEQFRHLPMPFHWKQEELKFKTGLRRLQHRVGEIHLLREALQK |
| | GAEAGQVSLHSLIETPANGTGPSEALAMLLQETTGELEAAKALVLKRIQIWKRQQQLAGNGAPFEESLAPLQERCES |
| | LVDIYSQLQQEVGAAGGELEPKTRASLTGRLDEVLRTLVTSCFLVEKQPPQVLKTQTKFQAGVRFLLGLRFLGAPAKP |
| | PLVRADMVTEKQARELSVPQGPGAGAESTGEIINNTVPLENSIPGNCCSALFKNLLLKKIKRCERKGTESVTEEKCAVL |
| | FSASFTLGPGKLPIQLQALDLPLVVIVHGNQDNNAKATILWDNAFSEMDRVPFVVAERVPWEKMCETLNLKFMAE |
| | VGTNRGLLPEHFLFLAQKIFNDNSLSMEAFQHRSVSWSQFNKEILLGRGFTFWQWFDGVLDLTKRCLRSYWSDRLII |
| | GFISKQYAASLLLNEPDGTFLLRFSDSEIGGITIAHVIRGQDGSPQIENIQPFSAKDLSIRSLGDRIRDLAQLKNLYPKKPK |
| | DEAFRSHYKPEQMGKDGRGYVPATIKMTVERDQPLPTPELQMPTMVPSYDLGMAPDSSMSMQLGPDMVPQVY |
| | PPHSHSIPPYQGLSPEESVNVLSAFQEPHLQMPPSLGQMSLPFDQPHPQGLLPCQPQEHAVSSPDPLLCSDVTMVE |
| | DSCLSQPVTAFPQGTWIGEDIFPPLLPPTEQDLTKLLLEGQGESGGGSLGAQPLLQPSHYGQSGISMSHMDLRANPS |
| | W |
| | (STAT6 (S407D/V547A/T548A); P010 with no epitope tag) |
| 140 | MSLWGLVSKMPPEKVQRLYVDFPQHLRHLLGDWLESQPWEFLVGSDAFCCNLASALLSDTVQHLQASVGEQGEG |
| | STILQHISTLESIYQRDPLKLVATFRQILQGEKKAVMEQFRHLPMPFHWKQEELKFKTGLRRLQHRVGEIHLLREALQK |
| | GAEAGQVSLHSLIETPANGTGPSEALAMLLQETTGELEAAKALVLKRIQIWKRQQQLAGNGAPFEESLAPLQERCES |
| | LVDIYSQLQQEVGAAGGELEPKTRASLTGRLDEVLRTLVTSCFLVEKQPPQVLKTQTKFQAGVRFLLGLRFLGAPAKP |
| | PLVRADMVTEKQARELSVPQGPGAGAESTGEIINNTVPLENSIPGNCCSALFKNLLLKKIKRCERKGTESVTEEKCAVL |
| | FSASFTLGPGKLPIQLQALSLPLVVIVHGNQDNNAKATILWDNAFSEMDRVPFVVAERVPWEKMCETLNLKFMAEV |
| | GTNRGLLPEHFLFLAQKIFNDNSLSMEAFQHRSVSWSQFNKEILLGRGFTFWQWFDGVLDLTKRCLRSYWSDRLIIG |
| | FISKQYAASLLLNEPDGTFLLRFSDSEIGGITIAHVIRGQDGSPQIENIQPFSAKDLSIRSLGDRIRDLAQLKNLYPKKPKD |
| | EAFRSHYKPEQMGKDGRGFVPATIKMTVERDQPLPTPELQMPTMVPSYDLGMAPDSSMSMQLGPDMVPQVYP |
| | PHSHSIPPYQGLSPEESVNVLSAFQEPHLQMPPSLGQMSLPFDQPHPQGLLPCQPQEHAVSSPDPLLCSDVTMVED |
| | SCLSQPVTAFPQGTWIGEDIFPPLLPPTEQDLTKLLLEGQGESGGGSLGAQPLLQPSHYGQSGISMSHMDLRANPS |
| | W |
| | (STAT6 (V547A/T548A/Y641F); P011 with no epitope tag) |
| 141 | SAEVIHQVEEALDTDEKEMLLFLCRDVAIDVVPPNVRDLLDILRERGKLSVGDLAELLYRVRRFDLLKRILKMDRKAVE |
| | THLLRNPHLVSDYRVLMAEIGEDLDKSDVSSLIFLMKDYMGRGKISKEKSFLDLVVELEKLNLVAPDQLDLLEKCLKNI |
| | HRIDLKTKIQKYKQSVQGAGTSYRNVLQAAIQKSLKDPSNNFRLHNGRSKEQRLKEQLGAQQEPVKKSIQESEAFLP |
| | QSIPEERYKMKSKPLGICLIIDCIGNETELLRDTFTSLGYEVQKFLHLSMHGISQILGQFACMPEHRDYDSFVCVLVSRG |
| | GSQSVYGVDQTHSGLPLHHIRRMFMGDSCPYLAGKPKMFFIQNYVVSEGQLEDSSLLEVDGPAMKNVEFKAQKRG |
| | LCTVHREADFFWSLCTADMSLLEQSHSSPSLYLQCLSQKLRQERKRPLLDLHIELNGYMYDWNSRVSAKEKYYVWL |
| | QHTLRKKLILSYT |
| | (hu-cFLIP-L; P1006 without epitope tag) |
| 142 | SAEVIHQVEEALDTDEKEMLLFLCRDVAIDVVPPNVRDLLDILRERGKLSVGDLAELLYRVRRFDLLKRILKMDRKAVE |
| | THLLRNPHLVSDYRVLMAEIGEDLDKSDVSSLIFLMKDYMGRGKISKEKSFLDLVVELEKLNLVAPDQLDLLEKCLKNI |
| | HRIDLKTKIQKYKQSVQGAGTSYRNVLQAAIQKSLKDPSNNFRLHNGRSKEQRLKEQLGAQQEPVKKS |
| | (hu-cFLIP-S(1-227); P1007 without epitope tag) |
| 143 | SAEVIHQVEEALDTDEKEMLLFLCRDVAIDVVPPNVRDLLDILRERGKLSVGDLAELLYRVRRFDLLKRILKMDRKAVE |
| | THLLRNPHLVSDYRVLMAEIGEDLDKSDVSSLIFLMKDYMGRGKISKEKSFLDLVVELEKLNLVAPDQLDLLEKCLKNI |
| | HRIDLKTKIQKYKQSVQGAGTSYRNVLQAAIQKSLKD |
| | (hu-cFLIP-p22(1-198); P1008 without epitope tag) |
| 144 | SAEVIHQVEEALDTDEKEMLLFLCRDVAIDVVPPNVRDLLDILRERGKLSVGDLAELLYRVRRFDLLKRILKMDRKAVE |
| | THLLRNPHLVSDYRVLMAEIGEDLDKSDVSSLIFLMKDYMGRGKISKEKSFLDLVVELEKLNLVAPDQLDLLEKCLKNI |
| | HRIDLKTKIQKYKQSVQGAGTSYRNVLQAAIQKSLKDPSNNFRLHNGRSKEQRLKEQLGAQQEPVKKSIQESEAFLP |
| | QSIPEERYKMKSKPLGICLIIDCIGNETELLRDTFTSLGYEVQKFLHLSMHGISQILGQFACMPEHRDYDSFVCVLVSRG |
| | GSQSVYGVDQTHSGLPLHHIRRMFMGDSCPYLAGKPKMFFIQNYVVSEGQLEDSSLLEVD |
| | (hu-cFLIP-p43(1-376); P1009 without epitope tag) |
| 145 | GPAMKNVEFKAQKRGLCTVHREADFFWSLCTADMSLLEQSHSSPSLYLQCLSQKLRQERKRPLLDLHIELNGYMYD |
| | WNSRVSAKEKYYVWLQHTLRKKLILSYT |
| | (hu-cFLIP-p12(377-480); P1010 without epitope tag) |
| 146 | MSWSPSLTTQTCGAWEMKERLGTGGFGNVIRWHNQETGEQIAIKQCRQELSPRNRERWCLEIQIMRRLTH |
| | PNVVAARDVPEGMQNLAPNDLPLLAMEYCQGGDLRKYLNQFENCCGLREGAILTLLSDIASALRYLHEN |
| | RIIHRDLKPENIVLQQGEQRLIHKIIDLGYAKELDQGELCTEFVGTLQYLAPELLEQQKYTVTVDYWSFGTLAF |
| | ECITGFRPFLPNWQPVQWHSKVRQKSEVDIVVSEDLNGTVKFSSSLPYPNNLNSVLAERLEKWLQLMLM |
| | WHPRQRGTDPTYGPNGCFKALDDILNLKLVHILNMVTGTIHTYPVTEDESLQSLKARIQQDTGIPEEDQELL |
| | QEAGLALIPDKPATQCISDGKLNEGHTLDMDLVFLFDNSKITYETQISPRPQPESVSCILQEPKRNLAFFQLRK |
| | VWGQVWHSIQTLKEDCNRLQQGQRAAMMNLLRNNSCLSKMKNSMASMSQQLKAKLDFFKTSIQIDLEK |
| | YSEQTEFGITSDKLLLAWREMEQAVELCGRENEVKLLVERMMALQTDIVDLQRSPMGRKQGGTLDDLEEQ |
| | ARELYRRLREKPRDQRTEGDSQEMVRLLLQAIQSFEKKVRVIYTQLSKTVVCKQKALELLPKVEEVVSLMN |
| | EDEKTVVRLQEKRQKELWNLLKIACSKVRGPVSGSPDSMNASRLSQPGQLMSQPSTASNSLPEPAKKSEEL |
| | VAEAHNLCTLLENAIQDTVREQDQSFTALDWSWLQTEEEEHSCLEQAS |
| | (huIKK2ca(S177E/S181E); P4005 without epitope tag) |
| 147 | MSWSPSLTTQTCGAWEMKERLGTGGFGNVIRWHNQETGEQIAIKQCRQELSPRNRERWCLEIQIMRRLTH |
| | PNVVAARDVPEGMQNLAPNDLPLLAMEYCQGGDLRKYLNQFENCCGLREGAILTLLSDIASALRYLHEN |
| | RIIHRDLKPENIVLQQGEQRLIHKIIDLGYAKELDQGALCTAFVGTLQYLAPELLEQQKYTVTVDYWSFGTLAF |
| | ECITGFRPFLPNWQPVQWHSKVRQKSEVDIVVSEDLNGTVKFSSSLPYPNNLNSVLAERLEKWLQLMLM |
| | WHPRQRGTDPTYGPNGCFKALDDILNLKLVHILNMVTGTIHTYPVTEDESLQSLKARIQQDTGIPEEDQELL |
| | QEAGLALIPDKPATQCISDGKLNEGHTLDMDLVFLFDNSKITYETQISPRPQPESVSCILQEPKRNLAFFQLRK |
| | VWGQVWHSIQTLKEDCNRLQQGQRAAMMNLLRNNSCLSKMKNSMASMSQQLKAKLDFFKTSIQIDLEK |
| | YSEQTEFGITSDKLLLAWREMEQAVELCGRENEVKLLVERMMALQTDIVDLQRSPMGRKQGGTLDDLEEQ |
| | ARELYRRLREKPRDQRTEGDSQEMVRLLLQAIQSFEKKVRVIYTQLSKTVVCKQKALELLPKVEEVVSLMN |
| | EDEKTVVRLQEKRQKELWNLLKIACSKVRGPVSGSPDSMNASRLSQPGQLMSQPSTASNSLPEPAKKSEEL |
| | VAEAHNLCTLLENAIQDTVREQDQSFTALDWSWLQTEEEEHSCLEQAS |
| | (huIKK2null(S177A/S181A); P4006 without epitope tag) |
| 148 | MSWSPSLPTQTCGAWEMKERLGTGGFGNVIRWHNQATGEQIAIKQCRQELSPKNRNRWCLEIQIMRRLNHPN |
| | VVAARDVPEGMQNLAPNDLPLLAMEYCQGGDLRRYLNQFENCCGLREGAVLTLLSDIASALRYLHENRIIHRDLK |
| | PENIVLQQGEKRLIHKIIDLGYAKELDQGELCTEFVGTLQYLAPELLEQQKYTVTVDYWSFGTLAFECITGFRPFLPN |
| | WQPVQWHSKVRQKSEVDIVVSEDLNGAVKFSSSLPFPNNLNSVLAERLEKWLQLMLMWHPRQRGTDPQYGP |
| | NGCFRALDDILNLKLVHVLNMVTGTVHTYPVTEDESLQSLKTRIQENTGILETDQELLQKAGLVLLPDKPATQCISD |
| | SKTNEGLTLDMDLVFLLDNSKINYETQITPRPPPESVSCILQEPKRNLSFFQLRKVWGQVWHSIQTLKEDCNRLQQ |
| | GQRAAMMSLLRNNSCLSKMKNAMASTAQQLKAKLDFFKTSIQIDLEKYKEQTEFGITSDKLLLAWREMEQAVEQ |
| | CGRENDVKHLVERMMALQTDIVDLQRSPMGRKQGGTLDDLEEQARELYRKLREKPRDQRTEGDSQEMVRLLLQ |
| | AIQSFEKKVRVIYTQLSKTVVCKQKALELLPKVEEVVSLMNEDERTVVRLQEKRQKELWNLLKIACSKVRGPVSGSP |
| | DSMNVSRLSHPGQLMSQPSSACDSLPESDKKSEELVAEAHALCSRLESALQDTVKEQDRSFTTLDWSWLQMEDEE |
| | RCSLEQACD |
| | (muIKK2ca(S177E/S181E); P4002 without epitope tag) |
| 149 | MSWSPSLPTQTCGAWEMKERLGTGGFGNVIRWHNQATGEQIAIKQCRQELSPKNRNRWCLEIQIMRRLNHPN |
| | VVAARDVPEGMQNLAPNDLPLLAMEYCQGGDLRRYLNQFENCCGLREGAVLTLLSDIASALRYLHENRIIHRDLK |
| | PENIVLQQGEKRLIHKIIDLGYAKELDQGALCTAFVGTLQYLAPELLEQQKYTVTVDYWSFGTLAFECITGFRPFLPN |
| | WQPVQWHSKVRQKSEVDIVVSEDLNGAVKFSSSLPFPNNLNSVLAERLEKWLQLMLMWHPRQRGTDPQYGP |
| | NGCFRALDDILNLKLVHVLNMVTGTVHTYPVTEDESLQSLKTRIQENTGILETDQELLQKAGLVLLPDKPATQCISD |
| | SKTNEGLTLDMDLVFLLDNSKINYETQITPRPPPESVSCILQEPKRNLSFFQLRKVWGQVWHSIQTLKEDCNRLQQ |
| | GQRAAMMSLLRNNSCLSKMKNAMASTAQQLKAKLDFFKTSIQIDLEKYKEQTEFGITSDKLLLAWREMEQAVEQ |
| | CGRENDVKHLVERMMALQTDIVDLQRSPMGRKQGGTLDDLEEQARELYRKLREKPRDQRTEGDSQEMVRLLLQ |
| | AIQSFEKKVRVIYTQLSKTVVCKQKALELLPKVEEVVSLMNEDERTVVRLQEKRQKELWNLLKIACSKVRGPVSGSP |
| | DSMNVSRLSHPGQLMSQPSSACDSLPESDKKSEELVAEAHALCSRLESALQDTVKEQDRSFTTLDWSWLQMEDEE |
| | RCSLEQACD |
| | muIKK2null(S177A/S181A); P4003 without epitope tag) |
| 150 | MERPPGLRPGAGGPWEMRERLGTGGFGNVCLYQHRELDLKIAIKSCRLELSTKNRERWCHEIQIMKKLNHANVVK |
| | ACDVPEELNILIHDVPLLAMEYCSGGDLRKLINKPENCCGLKESQILSLLSDIGSGIRYLHENKIIHRDLKPENIVLQDVG |
| | GKIIHKIIDLGYAKDVDQGELCTEFVGTLQYLAPELFENKPYTATVDYWSFGTMVFECIAGYRPFLHHLQPFTWHEKIK |
| | KKDPKCIFACEEMSGEVRFSSHLPQPNSLCSLVVEPMENWLQLMLNWDPQQRGGPVDLTLKQPRCFVLMDHILNL |
| | KIVHILNMTSAKIISFLLPPDESLHSLQSRIERETGINTGSQELLSETGISLDPRKPASQCVLDGVRGCDSYMVYLFDKSK |
| | TVYEGPFASRSLSDCVNYIVQDSKIQLPIIQLRKVWAEAVHYVSGLKEDYSRLFQGQRAAMLSLLRYNANLTKMKNTL |
| | ISASQQLKAKLEFFHKSIQLDLERYSEQMTYGISSEKMLKAWKEMEEKAIHYAEVGVIGYLEDQIMSLHAEIMELQKS |
| | PYRRQGDLMESLEQRAIDLYKQLKHRPSDHSYSDSTEMVKIIVHTVQSQDRVLKELFGHLSKLLGCKQKHIDLLPKVEV |
| | ALSNIKEADNTVMFMQGKRQKEIWHLLKIACTQAAARALVGAALEGAVAPQAAAWLPPAAAEHDHALACVVAPQ |
| | DGEAAAQMIEENINCLGHLAAIIHEANEEQGNSMMNLDWSWLTE |
| | Human constitutively active IKK alpha (PEST mutation) P.4013/4014 without epitope |
| | tag-amino acid |
| 151 | ATGGAACGCCCCCCTGGACTGAGGCCTGGAGCAGGAGGACCCTGGGAAATGCGCGAACGGCTGGGTACTGGT |
| | GGTTTCGGCAACGTGTGCCTCTACCAGCATCGGGAGTTGGACCTGAAGATCGCCATCAAGTCCTGCCGCCTGG |
| | AGCTGTCGACCAAGAACCGGGAACGCTGGTGTCATGAAATCCAGATTATGAAAAAGCTGAACCACGCTAACGT |
| | GGTCAAAGCTTGCGACGTGCCCGAAGAACTGAATATCCTGATCCACGATGTGCCCCTCCTCGCAATGGAGTACT |
| | GCAGCGGAGGCGATCTCCGGAAGCTGCTCAACAAGCCGGAGAACTGCTGTGGCCTTAAAGAGAGCCAGATTC |
| | TGAGCCTTCTGTCGGACATCGGCTCGGGTATCCGATATCTTCACGAGAACAAGATTATTCACAGAGATCTGAAG |
| | CCAGAGAACATCGTGCTGCAAGATGTCGGAGGAAAGATCATTCATAAGATCATCGACCTGGGATACGCCAAGG |
| | ACGTGGATCAAGGCGAACTGTGCACCGAATTCGTGGGAACCCTCCAGTACCTGGCCCCGGAACTGTTCGAAAA |
| | CAAACCCTACACCGCCACCGTGGACTACTGGTCCTTTGGAACTATGGTGTTCGAGTGTATAGCTGGCTACCGGC |
| | CATTTCTCCATCACTTGCAGCCCTTCACCTGGCACGAAAAGATCAAGAAGAAGGACCCCAAGTGCATTTTCGCG |
| | TGCGAAGAGATGTCGGGGGAAGTGCGCTTCTCGTCCCACTTGCCCCAGCCCAACTCCCTGTGCTCCCTGGTGGT |
| | CGAACCGATGGAAAACTGGCTGCAACTGATGCTGAACTGGGATCCTCAACAGCGCGGTGGACCAGTGGATCTG |
| | ACTCTGAAGCAGCCCAGATGCTTCGTGCTGATGGACCATATCCTGAACCTCAAGATCGTCCACATCCTGAACAT |
| | GACCTCCGCCAAGATCATTTCCTTCCTCCTCCCGCCCGATGAGAGCCTGCACTCACTGCAGTCCAGAATCGAGA |
| | GGGAAACCGGTATTAACACTGGGTCACAGGAACTCCTGTCCGAAACCGGAATCTCTCTGGACCCTCGCAAGCC |
| | AGCATCCCAGTGCGTCCTGGATGGGGTCAGGGGATGCGACTCGTACATGGTCTACCTCTTCGATAAGTCAAAG |
| | ACCGTCTACGAGGGACCCTTTGCCAGCCGGAGCCTGTCAGACTGCGTGAACTACATCGTGCAGGACTCTAAGA |
| | TTCAGCTGCCAATTATCCAGCTCCGGAAAGTCTGGGCAGAAGCGGTGCACTACGTGTCCGGACTGAAAGAGGA |
| | CTACTCCCGGCTGTTCCAGGGCCAGAGGGCAGCCATGCTGTCCCTGCTCCGCTACAACGCCAACCTCACGAAGA |
| | TGAAGAACACCCTGATCTCCGCGTCACAACAACTGAAGGCCAAGCTGGAATTCTTCCACAAGTCCATTCAATTG |
| | GATCTGGAGCGGTACTCCGAGCAGATGACTTACGGCATTAGCTCCGAAAAGATGCTCAAGGCCTGGAAGGAG |
| | ATGGAGGAGAAGGCCATTCATTATGCCGAAGTGGGGGTGATCGGATACCTGGAGGATCAGATCATGTCCCTTC |
| | ATGCCGAGATTATGGAACTCCAGAAGTCCCCGTACCGGAGGCAGGGCGATTTGATGGAGAGCTTGGAACAAC |
| | GCGCCATCGACCTGTACAAGCAGCTCAAGCACAGACCGAGCGACCACTCGTACTCCGACTCGACTGAGATGGT |
| | GAAAATTATCGTGCACACCGTGCAGTCCCAAGACCGGGTCCTGAAGGAGCTGTTCGGACACCTGAGCAAGCTG |
| | CTGGGGTGCAAGCAAAAGATCATTGACCTTCTGCCAAAAGTGGAGGTGGCCCTGAGCAACATTAAGGAAGCC |
| | GACAACACCGTGATGTTCATGCAGGGCAAGCGGCAGAAGGAGATCTGGCATCTTCTCAAGATCGCGTGTACCC |
| | AGGCTGCAGCGAGAGCCTTGGTGGGCGCTGCCCTGGAAGGTGCCGTGGCACCACAGGCCGCTGCTTGGCTGC |
| | CTCCTGCTGCTGCTGAGCACGATCACGCACTGGCCTGCGTGGTGGCACCGCAGGACGGAGAGGCTGCCGCGC |
| | AGATGATCGAGGAAAACCTGAACTGCCTGGGTCACCTGGCTGCCATCATCCACGAAGCCAACGAGGAGCAAG |
| | GAAACAGCATGATGAATCTCGACTGGAGCTGGCTGACTGAG |
| | Human constitutively active IKK alpha (PEST mutation) P.4013/4014 without epitope |
| | tag-nucleotide |
| 152 | MSWSPSLTTQTCGAWEMKERLGTGGFGNVIRWHNQETGEQIAIKQCRQELSPRNRERWCLEIQIMRRLTHPNVV |
| | AARDVPEGMQNLAPNDLPLLAMEYCQGGDLRKYLNQFENCCGLREGAILTLLSDIASALRYLHENRIIHRDLKPENIV |
| | LQQGEQRLIHKIIDLGYAKELDQGELCTEFVGTLQYLAPELLEQQKYTVTVDYWSFGTLAFECITGFRPFLPNWQPVQ |
| | WHSKVRQKSEVDIVVSEDLNGTVKFSSSLPYPNNLNSVLAERLEKWLQLMLMWHPRQRGTDPTYGPNGCFKALD |
| | DILNLKLVHILNMVTGTIHTYPVTEDESLQSLKARIQQDTGIPEEDQELLQEAGLALIPDKPATQCISDGKLNEGHTLD |
| | MDLVFLFDNSKITYETQISPRPQPESVSCILQEPKRNLAFFQLRKVWGQVWHSIQTLKEDCNRLQQGQRAAMMNL |
| | LRNNSCLSKMKNSMASMSQQLKAKLDFFKTSIQIDLEKYSEQTEFGITSDKLLLAWREMEQAVELCGRENEVKLLVE |
| | RMMALQTDIVDLQRSPMGRKQGGTLDDLEEQARELYRRLREKPRDQRTEGDSQEMVRLLLQAIQSFEKKVRVIYT |
| | QLSKTVVCKQKALELLPKVEEVVSLMNEDEKTVVRLQEKRQKELWNLLKIACSKVRGPVAGAPDAMNAARLAQPG |
| | QLMAQPATAANALPEPAKKAEELVAEAHNLCTLLENAIQDTVREQDQSFTALDWSWLQTEEEEHSCLEQAS |
| | Human constitutively active IKK beta (PEST mutation) P.4015/4016 without epitope |
| | tag-amino acid |
| 153 | ATGTCGTGGTCCCCCTCACTTACTACTCAAACTTGCGGCGCCTGGGAAATGAAGGAAAGACTCGGTACCGGGG |
| | GATTTGGAAACGTGATCCGGTGGCACAACCAAGAAACCGGAGAGCAAATTGCGATCAAGCAGTGTAGACAGG |
| | AACTGAGCCCTCGGAACAGAGAGCGGTGGTGCCTGGAGATTCAGATTATGCGCCGGCTGACCCATCCGAACGT |
| | GGTGGCTGCCAGGGATGTCCCGGAGGGCATGCAGAACCTGGCCCCTAACGACCTCCCACTCCTGGCCATGGAA |
| | TACTGCCAGGGTGGCGATCTGCGGAAGTACCTTAACCAATTCGAAAACTGCTGTGGACTCAGGGAAGGGGCCA |
| | TTCTGACTCTCTTGTCGGACATCGCCAGCGCCCTGAGATACCTCCACGAGAACAGAATCATCCATCGCGATCTG |
| | AAGCCGGAGAACATTGTGCTGCAACAGGGCGAACAGCGGCTGATCCACAAAATCATTGATCTCGGATATGCCA |
| | AGGAACTGGACCAGGGCGAACTCTGCACCGAATTCGTGGGCACTCTCCAGTACCTGGCACCCGAGTTGCTGGA |
| | GCAGCAGAAGTACACCGTCACCGTCGACTACTGGTCCTTCGGAACCCTCGCATTCGAATGTATCACTGGCTTCC |
| | GCCCTTTCCTGCCTAACTGGCAGCCTGTGCAGTGGCATTCGAAGGTCCGGCAGAAATCGGAGGTGGACATCGT |
| | GGTGTCCGAGGATCTGAACGGCACAGTGAAGTTCTCCTCCTCACTGCCTTACCCCAACAACCTCAACTCCGTGCT |
| | GGCCGAACGGCTGGAAAAGTGGCTCCAGCTTATGCTGATGTGGCATCCACGCCAGCGGGGTACTGATCCGACC |
| | TACGGTCCGAACGGGTGCTTCAAGGCCCTGGACGACATACTGAACCTCAAGCTCGTGCACATCCTCAATATGGT |
| | GACCGGCACGATCCATACTTACCCCGTCACCGAGGACGAATCGTTGCAGTCACTGAAGGCTCGGATCCAGCAG |
| | GACACCGGGATTCCCGAAGAGGACCAGGAACTTCTGCAGGAAGCGGGACTGGCGTTGATCCCCGACAAGCCT |
| | GCCACCCAGTGCATCTCTGACGGGAAGCTGAATGAAGGTCACACCCTGGATATGGACCTTGTGTTCCTGTTCGA |
| | CAATTCCAAGATCACCTACGAGACTCAGATTAGCCCTAGGCCTCAGCCGGAATCCGTGTCGTGCATCCTGCAAG |
| | AACCGAAGCGGAATCTGGCGTTCTTTCAACTGCGGAAAGTGTGGGGCCAAGTCTGGCACAGCATTCAGACACT |
| | GAAGGAGGATTGCAACCGGCTGCAGCAAGGACAGCGCGCCGCTATGATGAATCTGCTGCGCAACAATTCCTGC |
| | CTCTCAAAAATGAAGAACTCCATGGCCTCGATGTCCCAGCAATTGAAGGCCAAGCTGGATTTCTTCAAGACCTC |
| | GATCCAGATCGACCTGGAAAAGTACAGCGAGCAGACCGAGTTCGGAATCACCTCCGACAAGCTGCTGTTGGCA |
| | TGGCGGGAGATGGAACAAGCGGTGGAGCTGTGCGGACGCGAAAACGAGGTCAAACTGTTGGTGGAAAGAAT |
| | GATGGCCCTGCAGACCGACATCGTGGACCTCCAGCGATCCCCTATGGGCCGGAAGCAGGGTGGCACCCTCGAT |
| | GACCTGGAAGAACAGGCTCGGGAGCTGTACAGGCGCCTGCGGGAAAAGCCGCGGGACCAGAGAACTGAAGG |
| | GGATTCCCAGGAGATGGTGCGCCTGCTGCTTCAAGCCATCCAGTCATTCGAAAAGAAGGTCCGCGTGATCTAC |
| | ACCCAACTGAGCAAGACTGTGGTGTGCAAGCAGAAGGCCCTCGAACTGCTGCCGAAGGTGGAGGAGGTCGTG |
| | TCCCTGATGAACGAGGACGAAAAGACGGTCGTGAGACTCCAGGAAAAGAGACAGAAGGAACTGTGGAACCTT |
| | CTCAAGATTGCCTGCTCCAAAGTGCGCGGACCTGTGGCTGGAGCTCCCGACGCCATGAACGCCGCTAGACTCG |
| | CGCAGCCTGGACAGCTCATGGCCCAGCCCGCAACTGCAGCTAACGCCCTGCCCGAACCAGCGAAGAAGGCGG |
| | AGGAGCTTGTGGCGGAAGCCCACAACCTGTGCACCCTGCTCGAAAACGCCATCCAGGACACTGTGCGGGAACA |
| | AGACCAATCCTTCACCGCCCTGGATTGGTCATGGCTGCAGACTGAGGAAGAGGAGCACTCCTGTCTGGAGCAA |
| | GCCTCC |
| | Human constitutively active IKK beta (PEST mutation) P.4015/4016 without epitope |
| | tag-nucleotide |
| 154 | MERPPGLRPGAGGPWEMRERLGTGGFGNVSLYQHRELDLKIAIKSCRLELSSKNRERWCHEIQIMKKLDHANVVKA |
| | CDVPEELNFLINDVPLLAMEYCSGGDLRKLLNKPENCCGLKESQILSLLSDIGSGIRYLHENKIIHRDLKPENIVLQDVG |
| | GKTIHKIIDLGYAKDVDQGELCTEFVGTLQYLAPELFENKPYTATVDYWSFGTMVFECIAGYRPFLHHLQPFTWHEKI |
| | KKKDPKCIFACEEMTGEVRFSSHLPQPNSLCSLIVEPMESWLQLMLNWDPQQRGGPIDLTLKQPRCFALMDHILNL |
| | KIVHILNMTSAKIISFLLPCDESLHSLQSRIERETGINTGSQELLSETGISLDPRKPASQCVLDGVRGCDSYMVYLFDKSK |
| | TVYEGPFASRSLSDCVNYIVQDSKIQLPIIQLRKVWAEAVHYVSGLKEDYSRLFQGQRAAMLSLLRYNANLTKMKNTL |
| | ISASQQLKAKLEFFRKSIQLDLERYSEQMTYGISSEKMLKAWKEMEEKAIHYSEVGVIGYLEDQIMSLHTEIMELQKSP |
| | YGRRQGDLMESLEQRAIDLYKQLKHRPPDHLYSDSTEMVKIIVHTVQSQDRVLKELFGHLSKLLGCKQKIIDLLPKVEV |
| | ALSNIKEADNTVMFMQGKRQKEIWHLLKIACTQAAARALVGAALEGAVAPPVAAWLPPALADREHPLTCVVAPQD |
| | GEALAQMIEENLNCLGHLAAHIREANEDQSSSLMSLDWSWLAE |
| | Mouse constitutively active IKK alpha (PEST mutation) P.4017/4018 without epitope |
| | tag-amino acid |
| 155 | ATGGAAAGACCGCCTGGATTGCGACCTGGAGCCGGAGGACCCTGGGAAATGAGAGAGAGATTGGGTACTGG |
| | AGGCTTCGGAAATGTCTCGCTGTACCAGCACCGCGAGCTCGACCTGAAGATCGCGATCAAGTCCTGTCGCCTG |
| | GAGCTGTCCAGCAAGAACAGAGAGCGGTGGTGCCACGAGATCCAGATTATGAAGAAGCTGGACCATGCCAAC |
| | GTCGTGAAGGCTTGCGATGTCCCGGAGGAACTCAATTTCCTTATTAACGACGTGCCGCTTCTCGCGATGGAGTA |
| | CTGCTCAGGCGGCGACTTGCGCAAGCTGCTTAACAAGCCCGAAAACTGCTGCGGTCTGAAGGAATCCCAAATT |
| | CTGTCACTCCTGTCCGATATTGGCTCAGGAATCCGCTACCTTCATGAGAATAAGATCATCCACCGCGACCTGAA |
| | GCCTGAGAACATTGTGCTGCAGGATGTCGGGGGAAAGACTATCCACAAGATAATCGACCTGGGATACGCCAA |
| | GGACGTCGATCAAGGGGAACTGTGCACCGAATTCGTGGGGACTCTCCAGTACTTGGCCCCCGAACTGTTTGAA |
| | AACAAGCCCTACACCGCCACCGTGGATTACTGGTCCTTCGGGACTATGGTGTTCGAGTGTATTGCCGGCTATCG |
| | CCCCTTTCTGCACCACCTCCAGCCCTTTACTTGGCACGAAAAGATCAAGAAGAAGGATCCGAAGTGCATCTTCG |
| | CTTGCGAAGAGATGACCGGAGAAGTCCGGTTTTCCAGCCATCTGCCTCAGCCGAACTCCCTGTGTTCCCTGATT |
| | GTGGAACCCATGGAGAGCTGGTTGCAGCTCATGCTCAACTGGGATCCGCAGCAACGCGGTGGCCCAATCGATC |
| | TTACCCTTAAGCAGCCTCGGTGCTTCGCGCTGATGGACCACATCCTCAATCTGAAGATCGTGCACATCCTGAAC |
| | ATGACTTCCGCCAAGATCATCTCCTTCCTGCTGCCGTGCGACGAAAGCCTGCACTCACTGCAGAGCCGGATCGA |
| | ACGGGAGACAGGCATAAACACGGGATCGCAAGAACTGCTGTCCGAAACCGGCATCTCCCTGGACCCACGGAA |
| | GCCTGCCTCCCAATGCGTCCTGGACGGAGTGCGGGGTTGCGACTCATACATGGTGTACCTCTTCGATAAGTCAA |
| | AGACCGTGTATGAAGGACCCTTCGCCTCCCGCTCCCTGAGCGACTGCGTGAACTACATCGTGCAGGACTCGAA |
| | GATCCAGCTGCCGATTATCCAGCTTCGGAAGGTCTGGGCGGAGGCTGTGCACTACGTGTCCGGTTTGAAAGAG |
| | GATTATAGCCGCCTGTTCCAGGGACAGAGAGCCGCCATGCTGTCCCTCCTCCGGTACAACGCCAACCTGACCAA |
| | GATGAAGAACACCCTGATCAGCGCCTCGCAGCAGCTGAAGGCCAAGCTGGAGTTCTTCCGGAAGTCGATCCAG |
| | CTCGACCTCGAAAGGTACTCAGAACAGATGACCTACGGAATTTCCTCCGAGAAGATGCTGAAAGCCTGGAAGG |
| | AAATGGAGGAGAAGGCCATTCACTACTCCGAAGTGGGCGTCATTGGCTACTTGGAGGACCAAATCATGTCTCT |
| | GCACACCGAAATCATGGAACTCCAGAAGTCGCCTTACGGACGACGCCAAGGGGACCTGATGGAGAGCCTGGA |
| | ACAGCGGGCCATCGATCTGTACAAGCAACTGAAGCATAGGCCGCCCGACCATCTCTACTCCGACTCGACTGAAA |
| | TGGTGAAGATTATTGTGCATACAGTGCAGAGCCAGGACAGAGTGCTGAAGGAGCTGTTCGGCCACCTGTCCAA |
| | GCTCCTGGGTTGCAAGCAGAAGATTATCGATCTGTTGCCCAAGGTGGAAGTGGCCCTGTCTAACATCAAAGAA |
| | GCCGACAACACTGTGATGTTTATGCAAGGAAAGCGGCAGAAAGAAATCTGGCACCTTCTGAAAATCGCGTGCA |
| | CCCAGGCTGCAGCTAGGGCACTCGTGGGTGCAGCGCTTGAAGGCGCCGTGGCACCTCCTGTCGCTGCCTGGTT |
| | GCCACCCGCGCTTGCTGACAGAGAGCACCCACTGACTTGTGTGGTGGCCCCACAGGACGGAGAAGCACTGGCC |
| | CAGATGATTGAGGAGAACCTGAACTGTCTGGGACACCTTGCCGCCATTATCCGGGAGGCCAACGAGGACCAGT |
| | CCTCGTCCCTGATGTCCCTGGATTGGTCATGGCTCGCTGAA |
| | Mouse constitutively active IKK alpha (PEST mutation) P.4017/4018 without epitope |
| | tag-nucleotide |
| 156 | MSWSPSLPTQTCGAWEMKERLGTGGFGNVIRWHNQATGEQIAIKQCRQELSPKNRNRWCLEIQIMRRLNHPNV |
| | VAARDVPEGMQNLAPNDLPLLAMEYCQGGDLRRYLNQFENCCGLREGAVLTLLSDIASALRYLHENRIIHRDLKPEN |
| | IVLQQGEKRLIHKIIDLGYAKELDQGELCTEFVGTLQYLAPELLEQQKYTVTVDYWSFGTLAFECITGFRPFLPNWQPV |
| | QWHSKVRQKSEVDIVVSEDLNGAVKFSSSLPFPNNLNSVLAERLEKWLQLMLMWHPRQRGTDPQYGPNGCFRAL |
| | DDILNLKLVHVLNMVTGTVHTYPVTEDESLQSLKTRIQENTGILETDQELLQKAGLVLLPDKPATQCISDSKTNEGLTL |
| | DMDLVFLLDNSKINYETQITPRPPPESVSCILQEPKRNLSFFQLRKVWGQVWHSIQTLKEDCNRLQQGQRAAMMSL |
| | LRNNSCLSKMKNAMASTAQQLKAKLDFFKTSIQIDLEKYKEQTEFGITSDKLLLAWREMEQAVEQCGRENDVKHLV |
| | ERMMALQTDIVDLQRSPMGRKQGGTLDDLEEQARELYRKLREKPRDQRTEGDSQEMVRLLLQAIQSFEKKVRVIYT |
| | QLSKTVVCKQKALELLPKVEEVVSLMNEDERTVVRLQEKRQKELWNLLKIACSKVRGPVAGAPDAMNVARLAHPG |
| | QLMAQPASACDALPESDKKAEELVAEAHALCSRLESALQDTVKEQDRSFTTLDWSWLQMEDEERCSLEQACD |
| | Mouse constitutively active IKK beta (PEST mutation) P.4019/4020 without epitope |
| | tag-amino acid |
| 157 | ATGAGCTGGAGCCCTTCACTGCCAACCCAAACCTGTGGAGCCTGGGAAATGAAAGAAAGACTGGGAACCGGA |
| | GGTTTCGGCAACGTGATCCGCTGGCATAACCAGGCCACTGGGGAGCAGATTGCCATCAAGCAGTGCCGGCAG |
| | GAGCTGTCCCCGAAGAACCGCAACCGGTGGTGCCTGGAAATCCAGATCATGCGGCGGCTTAACCACCCCAACG |
| | TGGTCGCCGCGAGAGATGTGCCGGAGGGCATGCAAAACCTGGCCCCCAACGATCTCCCGCTGTTGGCGATGG |
| | AGTATTGCCAGGGTGGCGATCTGCGGCGCTACCTGAATCAATTCGAGAACTGCTGCGGTCTGCGCGAAGGAGC |
| | TGTGCTTACGCTGCTCTCGGACATCGCCTCGGCGCTGAGATACCTCCACGAAAATCGGATCATCCACCGAGATC |
| | TCAAGCCGGAAAACATTGTGCTTCAGCAAGGGGAAAAGCGCCTCATCCATAAGATCATCGATCTCGGCTACGC |
| | CAAGGAGTTGGACCAGGGGGAGCTCTGCACTGAATTCGTGGGAACTCTGCAGTACTTGGCGCCCGAACTGCTG |
| | GAGCAACAGAAGTACACTGTGACCGTGGACTACTGGTCCTTTGGAACCCTGGCCTTCGAGTGCATTACTGGCTT |
| | CCGGCCTTTCCTTCCAAACTGGCAGCCGGTGCAGTGGCACTCAAAGGTCCGCCAGAAGTCCGAAGTGGACATC |
| | GTGGTGTCCGAGGACTTGAACGGCGCCGTGAAGTTCTCGTCCTCCCTGCCCTTCCCGAACAACCTCAACTCCGT |
| | GCTGGCCGAGAGGCTGGAAAAGTGGCTGCAGCTTATGCTGATGTGGCACCCTAGACAGCGCGGAACTGATCC |
| | GCAGTACGGCCCGAACGGCTGTTTTAGGGCCCTGGACGACATTCTGAACCTGAAACTCGTCCACGTGCTTAACA |
| | TGGTCACCGGTACCGTCCATACCTATCCGGTCACCGAGGACGAATCCCTGCAGTCCCTCAAGACTCGGATTCAG |
| | GAGAATACCGGCATTCTGGAAACCGACCAGGAGCTGCTGCAGAAGGCCGGACTGGTGCTGCTCCCCGATAAG |
| | CCCGCAACCCAGTGCATCTCAGACTCCAAGACCAACGAGGGCCTGACTCTCGACATGGACCTGGTGTTCCTGCT |
| | CGACAACAGCAAGATCAACTACGAAACCCAAATTACCCCTAGACCACCACCTGAATCCGTGAGCTGCATACTGC |
| | AGGAGCCCAAGCGCAACCTCTCCTTCTTCCAACTCCGGAAGGTCTGGGGCCAAGTGTGGCACTCCATTCAGACT |
| | CTGAAGGAAGATTGTAACAGGCTGCAGCAGGGACAGAGAGCCGCCATGATGAGCCTTCTGAGGAACAACTCT |
| | TGCCTGTCAAAGATGAAGAACGCCATGGCTTCCACCGCGCAGCAGTTGAAGGCGAAGCTGGACTTCTTTAAGA |
| | CCTCCATCCAAATCGACCTGGAGAAGTACAAGGAACAGACTGAGTTCGGGATTACGAGCGATAAACTCCTGCT |
| | CGCTTGGCGGGAAATGGAGCAAGCAGTGGAGCAGTGCGGACGGGAGAACGACGTCAAGCATCTCGTGGAGC |
| | GGATGATGGCGCTGCAGACCGACATTGTCGACTTGCAGCGCTCTCCAATGGGACGGAAGCAGGGAGGGACTC |
| | TGGACGATCTGGAGGAACAGGCCCGGGAACTGTACAGAAAGCTGAGGGAGAAGCCCCGGGATCAAAGAACC |
| | GAAGGAGACTCGCAAGAGATGGTGCGCCTGCTGCTGCAGGCGATCCAGTCCTTCGAGAAGAAGGTCCGCGTG |
| | ATCTACACTCAGCTGTCCAAGACCGTGGTCTGTAAACAGAAGGCCCTGGAACTGCTCCCGAAAGTGGAAGAAG |
| | TGGTGTCGCTCATGAATGAGGACGAGAGAACCGTGGTGCGCCTCCAAGAAAAGCGGCAGAAGGAACTCTGGA |
| | ACCTCCTCAAGATTGCCTGCTCGAAAGTGCGGGGACCTGTGGCTGGTGCTCCTGACGCCATGAACGTGGCCAG |
| | GCTTGCTCACCCTGGCCAACTTATGGCCCAGCCTGCATCCGCCTGTGACGCACTGCCCGAGTCGGACAAGAAG |
| | GCCGAAGAACTGGTCGCCGAAGCCCACGCACTGTGCAGCCGCCTGGAAAGCGCGCTGCAGGACACCGTGAAG |
| | GAGCAGGACCGCAGCTTTACCACTCTTGATTGGTCCTGGCTGCAAATGGAGGACGAAGAACGGTGCTCCCTGG |
| | AACAGGCCTGCGAC |
| | Mouse constitutively active IKK beta (PEST mutation) P.4019/4020 without epitope |
| | tag-nucleotide |
| 158 | MQPDMSLNVIKMKSSDFLESAELDSGGFGKVSLCFHRTQGLMIMKTVYKGPNCIEHNEALLEEAKMMNRLRHSRV |
| | VKLLGVIIEEGKYSLVMEYMEKGNLMHVLKAEMSTPLSVKGRIILEIIEGMCYLHGKGVIHKDLKPENILVDNDFHIKIA |
| | DLGLASFKMWSKLNNEEHNELREVDGTAKKNGGTLYYMAPEHLNDVNAKPTEKSDVYSFAVVLWAIFANKEPYEN |
| | AICEQQLIMCIKSGNRPDVDDITEYCPREIISLMKLCWEANPEARPTFPGIEEKFRPFYLSQLEESVEEDVKSLKKEYSNE |
| | NAVVKRMQSLQLDCVAVPSSRSNSATEQPGSLHSSQGLGMGPVEESWFAPSLEHPQEENEPSLQSKLQDEANYHL |
| | YGSRMDRQTKQQPRQNVAYNREEERRRRVSHDPFAQQRPYENFQNTEGKGTAYSSAASHGNAVHQPSGLTSQP |
| | QVLYQNNGLYSSHGFGTRPLDPGTAGPRVWYRPIPSHMPSLHNIPVPETNYLGNTPTMPFSSLPPTDESIKYTIYNST |
| | GIQIGAYNYMEIGGTSSSGGIKKEIEAIKKEQEAIKKKIEAIEKEIEA |
| | (huRIPK1(1-555).IZ.TM; TH1021 without epitope tag) |
| 159 | MQPDMSLNVIKMKSSDFLESAELDSGGFGKVSLCFHRTQGLMIMKTVYKGPNCIEHNEALLEEAKMMNRLRHSRV |
| | VKLLGVIIEEGKYSLVMEYMEKGNLMHVLKAEMSTPLSVKGRIILEIIEGMCYLHGKGVIHKDLKPENILVDNDFHIKIA |
| | DLGLASFKMWSKLNNEEHNELREVDGTAKKNGGTLYYMAPEHLNDVNAKPTEKSDVYSFAVVLWAIFANKEPYEN |
| | AICEQQLIMCIKSGNRPDVDDITEYCPREIISLMKLCWEANPEARPTFPGIEEKFRPFYLSQLEESVEEDVKSLKKEYSNE |
| | NAVVKRMQSLQLDCVAVPSSRSNSATEQPGSLHSSQGLGMGPVEESWFAPSLEHPQEENEPSLQSKLQDEANYHL |
| | YGSRMDRQTKQQPRQNVAYNREEERRRRVSHDPFAQQRPYENFQNTEGKGTAYSSAASHGNAVHQPSGLTSQP |
| | QVLYQNNGLYSSHGFGTRPLDPGTAGPRVWYRPIPSHMPSLHNIPVPETNYLGNTPTMPFSSLPPTDESIKYTIYNST |
| | GIQIGAYNYMEIGGTSSSGSDGSGSGSGSITIRAAFLEKENTALRTEIAELEKEVGRCENIVSKYETRYGPL |
| | (huRIPK1(1-555).EE.DM; TH1022 without epitope tag) |
| 160 | MQPDMSLNVIKMKSSDFLESAELDSGGFGKVSLCFHRTQGLMIMKTVYKGPNCIEHNEALLEEAKMMNRLRHSRV |
| | VKLLGVIIEEGKYSLVMEYMEKGNLMHVLKAEMSTPLSVKGRIILEIIEGMCYLHGKGVIHKDLKPENILVDNDFHIKIA |
| | DLGLASFKMWSKLNNEEHNELREVDGTAKKNGGTLYYMAPEHLNDVNAKPTEKSDVYSFAVVLWAIFANKEPYEN |
| | AICEQQLIMCIKSGNRPDVDDITEYCPREIISLMKLCWEANPEARPTFPGIEEKFRPFYLSQLEESVEEDVKSLKKEYSNE |
| | NAVVKRMQSLQLDCVAVPSSRSNSATEQPGSLHSSQGLGMGPVEESWFAPSLEHPQEENEPSLQSKLQDEANYHL |
| | YGSRMDRQTKQQPRQNVAYNREEERRRRVSHDPFAQQRPYENFQNTEGKGTAYSSAASHGNAVHQPSGLTSQP |
| | QVLYQNNGLYSSHGFGTRPLDPGTAGPRVWYRPIPSHMPSLHNIPVPETNYLGNTPTMPFSSLPPTDESIKYTIYNST |
| | GIQIGAYNYMEIGGTSSSGSDGSGSGSGSLEIRAAFLEKENTALRTRAAELRKRVGRCRNIVSKYETRYGPL |
| | (huRIPK1(1-555).RR.DM; TH1023 without epitope tag) |
| 161 | MQPDMSLDNIKMASSDLLEKTDLDSGGFGKVSLCYHRSHGFVILKKVYTGPNRAEYNEVLLEEGKMMHRLRHSRV |
| | VKLLGIIIEEGNYSLVMEYMEKGNLMHVLKTQIDVPLSLKGRIIVEAIEGMCYLHDKGVIHKDLKPENILVDRDFHIKIA |
| | DLGVASFKTWSKLTKEKDNKQKEVSSTTKKNNGGTLYYMAPEHLNDINAKPTEKSDVYSFGIVLWAIFAKKEPYENVI |
| | CTEQFVICIKSGNRPNVEEILEYCPREIISLMERCWQAIPEDRPTFLGIEEEFRPFYLSHFEEYVEEDVASLKKEYPDQSP |
| | VLQRMFSLQHDCVPLPPSRSNSEQPGSLHSSQGLQMGPVEESWFSSSPEYPQDENDRSVQAKLQEEASYHAFGIFA |
| | EKQTKPQPRQNEAYNREEERKRRVSHDPFAQQRARENIKSAGARGHSDPSTTSRGIAVQQLSWPATQTVWNNGL |
| | YNQHGFGTTGTGVWYPPNLSQMYSTYKTPVPETNIPGSTPTMPYFSGPVADDLIKYTIFNSSGIQIGNHNYMDVGL |
| | NSQPPNNTCKEESTSGGIKKEIEAIKKEQEAIKKKIEAIEKEIEA |
| | (msRIPK1(1-555).IZ.TM; TH1024 without epitope tag) |
| 162 | MQPDMSLDNIKMASSDLLEKTDLDSGGFGKVSLCYHRSHGFVILKKVYTGPNRAEYNEVLLEEGKMMHRLRHSRV |
| | VKLLGIIIEEGNYSLVMEYMEKGNLMHVLKTQIDVPLSLKGRIIVEAIEGMCYLHDKGVIHKDLKPENILVDRDFHIKIA |
| | DLGVASFKTWSKLTKEKDNKQKEVSSTTKKNNGGTLYYMAPEHLNDINAKPTEKSDVYSFGIVLWAIFAKKEPYENVI |
| | CTEQFVICIKSGNRPNVEEILEYCPREIISLMERCWQAIPEDRPTFLGIEEEFRPFYLSHFEEYVEEDVASLKKEYPDQSP |
| | VLQRMFSLQHDCVPLPPSRSNSEQPGSLHSSQGLQMGPVEESWFSSSPEYPQDENDRSVQAKLQEEASYHAFGIFA |
| | EKQTKPQPRQNEAYNREEERKRRVSHDPFAQQRARENIKSAGARGHSDPSTTSRGIAVQQLSWPATQTVWNNGL |
| | YNQHGFGTTGTGVWYPPNLSQMYSTYKTPVPETNIPGSTPTMPYFSGPVADDLIKYTIFNSSGIQIGNHNYMDVGL |
| | NSQPPNNTCKEESTSGSDGSGSGSGSITIRAAFLEKENTALRTEIAELEKEVGRCENIVSKYETRYGPL |
| | (msRIPK1(1-555).EE.DM; TH1025 without epitope tag) |
| 163 | MQPDMSLDNIKMASSDLLEKTDLDSGGFGKVSLCYHRSHGFVILKKVYTGPNRAEYNEVLLEEGKMMHRLRHSRV |
| | VKLLGIIIEEGNYSLVMEYMEKGNLMHVLKTQIDVPLSLKGRIIVEAIEGMCYLHDKGVIHKDLKPENILVDRDFHIKIA |
| | DLGVASFKTWSKLTKEKDNKQKEVSSTTKKNNGGTLYYMAPEHLNDINAKPTEKSDVYSFGIVLWAIFAKKEPYENVI |
| | CTEQFVICIKSGNRPNVEEILEYCPREIISLMERCWQAIPEDRPTFLGIEEEFRPFYLSHFEEYVEEDVASLKKEYPDQSP |
| | VLQRMFSLQHDCVPLPPSRSNSEQPGSLHSSQGLQMGPVEESWFSSSPEYPQDENDRSVQAKLQEEASYHAFGIFA |
| | EKQTKPQPRQNEAYNREEERKRRVSHDPFAQQRARENIKSAGARGHSDPSTTSRGIAVQQLSWPATQTVWNNGL |
| | YNQHGFGTTGTGVWYPPNLSQMYSTYKTPVPETNIPGSTPTMPYFSGPVADDLIKYTIFNSSGIQIGNHNYMDVGL |
| | NSQPPNNTCKEESTSGSDGSGSGSGSLEIRAAFLEKENTALRTRAAELRKRVGRCRNIVSKYETRYGPL |
| | (msRIPK1(1-555). RR.DM; TH1026 without epitope tag) |
| 164 | MSTASAASSSSSSSAGEMIEAPSQVLNFEEIDYKEIEVEEVVGRGAFGVVCKAKWRAKDVAIKQIESESERKAFIVELR |
| | QLSRVNHPNIVKLYGACLNPVCLVMEYAEGGSLYNVLHGAEPLPYYTAAHAMSWCLQCSQGVAYLHSMQPKALIH |
| | RDLKPPNLLLVAGGTVLKICDFGTACDIQTHMTNNKGSAAWMAPEVFEGSNYSEKCDVFSWGIILWEVITRRKPFD |
| | EIGGPAFRIMWAVHNGTRPPLIKNLPKPIESLMTRCWSKDPSQRPSMEEIVKIMTHLMRYFPGADEPLQYPCQEFG |
| | GGGGQSPTLTLQSTNTHTQSSSSSSDGGLFRSRPAHSLPPGEDGRVEPYVDFAEFYRLWSVDHGEQSVVTAP |
| | (human TAK1-TAB1; P4031 without epitope tag) |
| 165 | MAALKSWLSRSVTSFFRYRQCLCVPVVANFKKRCFSELIRPWHKTVTIGFGVTLCAVPIAQKSEPHSLSSEALMRRAV |
| | SLVTDSTSTFLSQTTYALIEAITEYTKAVYTLTSLYRQYTSLLGKMNSEEEDEVWQVIIGARAEMTSKHQEYLKLETTW |
| | MTAVGLSEMAAEAAYQTGADQASITARNHIQLVKLQVEEVHQLSRKAETKLAEAQIEELRQKTQEEGEERAESEQE |
| | AYLRED |
| | (Diablo.1; without epitope tag) |
| 166 | MAALKSWLSRSVTSFFRYRQCLCVPVVANFKKRCFSELIRPWHKTVTIGFGVTLCAVPIAQKSEPHSLSSEALMRRAV |
| | SLVTDSTSTFLSQTTYALIEAITEYTKAVYTLTSLYRQYTSLLGKMNLEEEDEVWQVIIGARAEMTSKHQEYLKLETTW |
| | MTAVGLSEMAAEAAYQTGADQASITARNHIQLVKLQVEEVHQLSRKAETKLAEAQIEELRQKTQEEGEERAESEQE |
| | AYLRED |
| | (Diablo.1(S126L); without epitope tag) |
| 167 | MAVPIAQKSEPHSLSSEALMRRAVSLVTDSTSTFLSQTTYALIEAITEYTKAVYTLTSLYRQYTSLLGKMNSEEEDEVW |
| | QVIIGARAEMTSKHQEYLKLETTWMTAVGLSEMAAEAAYQTGADQASITARNHIQLVKLQVEEVHQLSRKAETKLA |
| | EAQIEELRQKTQEEGEERAESEQEAYLRED |
| | (Diablo.1(56-239); without epitope tag) |
| 168 | MAVPIAQKSEPHSLSSEALMRRAVSLVTDSTSTFLSQTTYALIEAITEYTKAVYTLTSLYRQYTSLLGKMNLEEEDEVW |
| | QVIIGARAEMTSKHQEYLKLETTWMTAVGLSEMAAEAAYQTGADQASITARNHIQLVKLQVEEVHQLSRKAETKLA |
| | EAQIEELRQKTQEEGEERAESEQEAYLRED |
| | (Diablo.1(56-239/S126L); without epitope tag) |
| 169 | MAALKSWLSRSVTSFFRYRQCLCVPVVANFKKRCFSELIRPWHKTVTIGFGVTLCAVPIAQAVYTLTSLYRQYTSLLGK |
| | MNSEEEDEVWQVIIGARAEMTSKHQEYLKLETTWMTAVGLSEMAAEAAYQTGADQASITARNHIQLVKLQVEEV |
| | HQLSRKAETKLAEAQIEELRQKTQEEGEERAESEQEAYLRED |
| | (Diablo.3; TH2003 without epitope tag) |
| 170 | MAALKSWLSRSVTSFFRYRQCLCVPVVANFKKRCFSELIRPWHKTVTIGFGVTLCAVPIAQAVYTLTSLYRQYTSLLGK |
| | MNLEEEDEVWQVIIGARAEMTSKHQEYLKLETTWMTAVGLSEMAAEAAYQTGADQASITARNHIQLVKLQVEEV |
| | HQLSRKAETKLAEAQIEELRQKTQEEGEERAESEQEAYLRED |
| | (Diablo.3(S82L); TH2001 without epitope tag) |
| 171 | MAVPIAQAVYTLTSLYRQYTSLLGKMNSEEEDEVWQVIIGARAEMTSKHQEYLKLETTWMTAVGLSEMAAEAAYQ |
| | TGADQASITARNHIQLVKLQVEEVHQLSRKAETKLAEAQIEELRQKTQEEGEERAESEQEAYLRED |
| | (Diablo.3(56-195); TH2002 without epitope tag) |
| 172 | MAVPIAQAVYTLTSLYRQYTSLLGKMNLEEEDEVWQVIIGARAEMTSKHQEYLKLETTWMTAVGLSEMAAEAAYQ |
| | TGADQASITARNHIQLVKLQVEEVHQLSRKAETKLAEAQIEELRQKTQEEGEERAESEQEAYLRED |
| | (Diablo.3(56-195/S82L); without epitope tag) |
| 173 | MAAVILESIFLKRSQQKKKTSPLNFKKRLFLLTVHKLSYYKYDFERGRRGSKKGSIDVEKITCVETVVPEKNPPPERQIPR |
| | RGEESSEMEQISIIERFPYPFQVVYDEGPLYVFSPTEELRKRWIHQLKNVIRYNSDLVQKYHPCFWIDGQYLCCSQTAK |
| | NAMGCQILENRNGSLKPGSSHRKTKKPLPPTPEEDQILKKPLPPEPAAAPVSTSELKKVVALYDYMPMNANDLQLRK |
| | GDEYFILEESNLPWWRARDKNGQEGYIPSNYVTEAEDSIEMYEWYSKHMTRSQAEQLLKQEGKEGGFIVRDSSKAG |
| | KYTVSVFAKSTGDPQGVIRHYVVCSTPQSQYYLAEKHLFSTIPELINYHQHNSAGLISRLKYPVSQQNKNAPSTAGLGY |
| | GSWEIDPKDLTFLKELGTGQFGVVKYGKWRGQYDVAIKMIKEGSMSEDEFIEEAKVMMNLSHEKLVQLYGVCTKQ |
| | RPIFIITEYMANGCLLNYLREMRHRFQTQQLLEMCKDVCEAMEYLESKQFLHRDLAARNCLVNDQGVVKVSDFGLS |
| | RYVLDDEYTSSVGSKFPVRWSPPEVLMYSKFSSKSDIWAFGVLMWEIYSLGKMPYERFTNSETAEHIAQGLRLYRPH |
| | LASEKVYTIMYSCWHEKADERPTFKILLSNILDVMDEES |
| | (Btk(E41K); P4029 without epitope tag) |
| 174 | MVTHSKFPAAGMSRPLDTSLRLKTFSSKSEYQLVVNAVRKLQESGFYWSAVTGGEANLLLSAEPAGTFLIRDSSDQR |
| | HFFTLSVKTQSGTKNLRIQCEGGSFSLQSDPRSTQPVPRFDCVLKLVHHYMPPPGAPSFPSPPTEPSSEVPEQPSAQP |
| | LPGSPPRRAYYIYSGGEKIPLVLSRPLSSNVATLQHLCRKTVNGHLDSYEKVTQLPGPIREFLDQYDAPL |
| | (SQCS3; P4030 without epitope tag) |
| 175 | MRMKQIEDKIEEILSKIYHIENEIARIKKLIGEADQTSGNYLNMQDSQGVLSSFPAPQAVQDNPAMPTSSGSEGNVKL |
| | CSLEEAQRIWKQKSAEIYPIMDKSSRTRLALIICNEEFDSIPRRTGAEVDITGMTMLLQNLGYSVDVKKNLTASDMTTE |
| | LEAFAHRPEHKTSDSTFLVFMSHGIREGICGKKHSEQVPDILQLNAIFNMLNTKNCPSLKDKPKVIIIQACRGDSPGVV |
| | WFKDSVGVSGNLSLPTTEEFEDDAIKKAHIEKDFIAFCSSTPDNVSWRHPTMGSVFIGRLIEHMQEYACSCDVEEIFR |
| | KVRFSFEQPDGRAQMPTTERVTLTRCFYLFPGH |
| | (IZ_hsCASP1 (self-activating human Caspase 1); P2024 without epitope tag) |
| 176 | MRMKQLEDKIEELLSKIYHLENEIARLKKLIGEADQTSGNYLNMQDSQGVLSSFPAPQAVQDNPAMPTSSGSEGNV |
| | KLCSLEEAQRIWKQKSAEIYPIMDKSSRTRLALIICNEEFDSIPRRTGAEVDITGMTMLLQNLGYSVDVKKNLTASDMT |
| | TELEAFAHRPEHKTSDSTFLVFMSHGIREGICGKKHSEQVPDILQLNAIFNMLNTKNCPSLKDKPKVIIIQACRGDSPG |
| | VVWFKDSVGVSGNLSLPTTEEFEDDAIKKAHIEKDFIAFCSSTPDNVSWRHPTMGSVFIGRLIEHMQEYACSCDVEEI |
| | FRKVRFSFEQPDGRAQMPTTERVTLTRCFYLFPGH |
| | (DM_hsCASP1 (self-activating human Caspase 1); P2025 without epitope tag) |
| 177 | MRMKQIEDKIEEILSKIYHIENEIARIKKLIGERSAPSAETFVATEDSKGGHPSSSETKEEQNKEDGTFPGLTGTLKFCPL |
| | EKAQKLWKENPSEIYPIMNTTTRTRLALIICNTEFQHLSPRVGAQVDLREMKLLLEDLGYTVKVKENLTALEMVKEVK |
| | EFAACPEHKTSDSTFLVFMSHGIQEGICGTTYSNEVSDILKVDTIFQMMNTLKCPSLKDKPKVIIIQACRGEKQGVVLL |
| | KDSVRDSEEDFLTDAIFEDDGIKKAHIEKDFIAFCSSTPDNVSWRHPVRGSLFIESLIKHMKEYAWSCDLEDIFRKVRFS |
| | FEQPEFRLQMPTADRVTLTKRFYLFPGH |
| | (IZ_mmCASP1 (self-activating mouse Caspase 1); P2026 without epitope tag) |
| 178 | MRMKQLEDKIEELLSKIYHLENEIARLKKLIGERSAPSAETFVATEDSKGGHPSSSETKEEQNKEDGTFPGLTGTLKFCP |
| | LEKAQKLWKENPSEIYPIMNTTTRTRLALIICNTEFQHLSPRVGAQVDLREMKLLLEDLGYTVKVKENLTALEMVKEV |
| | KEFAACPEHKTSDSTFLVFMSHGIQEGICGTTYSNEVSDILKVDTIFQMMNTLKCPSLKDKPKVIIIQACRGEKQGVVL |
| | LKDSVRDSEEDFLTDAIFEDDGIKKAHIEKDFIAFCSSTPDNVSWRHPVRGSLFIESLIKHMKEYAWSCDLEDIFRKVRF |
| | SFEQPEFRLQMPTADRVTLTKRFYLFPGH |
| | (DM_mmCASP1 (self-activating mouse Caspase 1); P2027 without epitope tag) |
| 179 | MHHHHHHHHHHGKPIPNPLLGLDSTGIPVHLELASMTNMELMSSIVHQQVFPTEAGQSLVISASIIVFNLLELEGDY |
| | RGRVLELFRAAQLANDVVLQIMELCGATR |
| | (ADR concate mer with HIS tag) |
| 180 | VVGADGVGK |
| | (KRAS G12D 9 mer) |
| 181 | VVGAVGVGK |
| | (KRAS G12V 9 mer) |
| 182 | VGAGDVGKS |
| | (KRAS G13D 9 mer) |
| 183 | VVGACGVGK |
| | (KRAS G12C 9 mer) |
| 184 | MKLVVVGACGVGKSA |
| | (KRAS G12C 15 mer) |
| 185 | ATGACCGAGTACAAGCTGGTGGTGGTGGGCGCCGACGGCGTGGGCAAGAGCGCCCTGACCATCCAGCTGATC |
| | CAG |
| | (KRAS G12D 25 mer nucleotide sequence) |
| 186 | ATGACCGAGTACAAGCTGGTGGTGGTGGGCGCCGTGGGCGTGGGCAAGAGCGCCCTGACCATCCAGCTGATC |
| | CAG |
| | (KRAS G12V 25 mer nucleotide sequence) |
| 187 | ATGACCGAGTACAAGCTGGTGGTGGTGGGCGCCGGCGACGTGGGCAAGAGCGCCCTGACCATCCAGCTGATC |
| | CAG |
| | (KRAS G13D 25 mer nucleotide sequence) |
| 188 | ATGACCGAGTACAAGTTAGTGGTTGTGGGCGCCGACGGCGTGGGCAAGAGCGCCCTCACCATCCAGCTTATCC |
| | AGATGACGGAATATAAGTTAGTAGTAGTGGGAGCCGACGGTGTCGGCAAGTCCGCTTTGACCATTCAACTTAT |
| | TCAGATGACAGAGTATAAGCTGGTCGTTGTAGGCGCAGACGGCGTTGGAAAGTCGGCACTGACGATCCAGTT |
| | GATCCAG |
| | (KRAS G12D 25 mer?3 nucleotide sequence) |
| 189 | ATGACCGAGTACAAGCTCGTCGTGGTGGGCGCCGTGGGCGTGGGCAAGAGCGCCCTAACCATCCAGTTGATCC |
| | AGATGACCGAATATAAGCTCGTGGTAGTCGGAGCGGTGGGCGTTGGCAAGTCAGCGCTAACAATACAACTAAT |
| | CCAAATGACCGAATACAAGCTAGTTGTAGTCGGTGCCGTCGGCGTTGGAAAGTCAGCCCTTACAATTCAGCTCA |
| | TTCAG |
| | (KRAS G12V 25 mer?3 nucleotide sequence) |
| 190 | ATGACCGAGTACAAGCTCGTAGTGGTTGGCGCCGGCGACGTGGGCAAGAGCGCCCTAACCATCCAGCTCATCC |
| | AGATGACAGAATATAAGCTTGTGGTTGTGGGAGCAGGAGACGTGGGAAAGAGTGCGTTGACGATTCAACTCA |
| | TACAGATGACCGAATACAAGTTGGTGGTGGTCGGCGCAGGTGACGTTGGTAAGTCTGCACTAACTATACAACT |
| | GATCCAG |
| | (KRAS G13D 25 mer?3 nucleotide sequence) |
| 191 | ATGACCGAGTACAAGCTGGTGGTGGTGGGCGCCTGCGGCGTGGGCAAGAGCGCCCTGACCATCCAGCTGATC |
| | CAG |
| | (KRAS G12C 25 mer nucleotide sequence) |
| 192 | ATGACCGAGTACAAGCTCGTGGTTGTTGGCGCCTGCGGCGTGGGCAAGAGCGCCCTCACCATCCAGCTCATCC |
| | AGATGACAGAGTATAAGTTAGTCGTTGTCGGAGCTTGCGGAGTTGGAAAGTCGGCGCTCACCATTCAACTCAT |
| | ACAAATGACAGAATATAAGTTAGTGGTGGTGGGTGCGTGTGGCGTTGGCAAGAGTGCGCTTACTATCCAGCTC |
| | ATTCAG |
| | (KRAS G12C 25 mer?3 nucleotide sequence) |
| 193 | ATGACCGAGTACAAGCTGGTGGTGGTGGGCGCCGGCGGCGTGGGCAAGAGCGCCCTGACCATCCAGCTGATC |
| | CAG |
| | (KRAS WT 25 mer nucleotide sequence) |
| 194 | GGGAAATAAGAGAGAAAAGAAGAGTAAGAAGAAATATAAGAGCCACC |
| | (5′ UTR sequence; no promoter) |
| 195 | MUEYKLVVVGADGVGKSALUIQLIQMUEYKLVVVGAVGVGKSALUIQLIQMUEYKLVVVGAGDVGKSALUIQLIQ |
| | (KRAS(G12D G12V G13D) 75 mer “3MUT” aa. seq) |
| 196 | ATGACCGAGTACAAGCTCGTTGTAGTCGGCGCCGACGGCGTGGGCAAGAGCGCCTTGACCATCCAGTTGATCC |
| | AGATGACCGAATATAAGTTGGTGGTGGTAGGCGCAGTGGGAGTTGGCAAGTCAGCACTCACAATTCAGCTCAT |
| | TCAAATGACAGAATACAAGTTAGTCGTTGTAGGAGCAGGCGACGTCGGCAAGAGTGCCTTAACCATTCAACTA |
| | ATCCAG |
| | (KRAS(G12D G12V G13D) 75 mer “3MUT” nt. seq) |
| 197 | MTEYKLVVVGADGVGKSALTIQLIQMTEYKLVVVGAVGVGKSALTIQLIQMTEYKLVVVGAGDVGKSALTIQLIQMT |
| | EYKLVVVGACGVGKSALTIQLIQ |
| | (KRAS(G12D G12V G13D G12C) 100 mer “4MUT” aa. seq) |
| 198 | ATGACCGAGTACAAGCTCGTGGTCGTCGGCGCCGACGGGGTAGGCAAGTCCGCTCTGACCATTCAGCTCATCC |
| | AGATGACGGAGTACAAACTCGTGGTAGTGGGAGCCGTGGGTGTGGGCAAGAGCGCGCTCACCATCCAACTCA |
| | TCCAAATGACCGAATATAAACTCGTCGTGGTGGGAGCCGGCGACGTGGGAAAGAGCGCCCTTACCATCCAGTT |
| | AATCCAGATGACAGAATACAAGCTGGTGGTGGTCGGTGCCTGCGGCGTGGGTAAGTCCGCCCTGACAATCCAG |
| | CTGATCCAG |
| | (KRAS(G12D G12V G13D G12C) 100 mer “4MUT” nt. seq) |
| 199 | ATGCCCCACAGTAGCCTCCACCCCAGCATCCCCTGCCCCAGAGGCCACGGCGCACAGAAGGCCGCCCTGGTGC |
| | TGCTGAGCGCCTGTCTGGTGACCCTGTGGGGTCTGGGCGAGCCCCCCGAGCACACCCTGCGGTACCTCGTGCT |
| | GCATCTGGCCAGCCTGCAGCTGGGCCTGCTGCTGAACGGCGTGTGCAGCCTGGCCGAAGAGCTGAGACACATC |
| | CACAGCAGATACAGAGGCTCCTACTGGAGAACCGTCAGAGCCTGCCTCGGCTGTCCCCTGAGAAGAGGCGCCC |
| | TGCTGCTCCTGAGCATCTACTTCTACTACAGCCTGCCCAACGCCGTGGGCCCCCCCTTCACCTGGATGCTGGCCC |
| | TGCTGGGCCTGAGCCAGGCCCTGAACATCCTGCTGGGCCTGAAGGGCTTGGCCCCCGCCGAGATCTCCGCCGT |
| | GTGCGAGAAGGGCAACTTCAACATGGCCCATGGCCTTGCCTGGTCCTACTACATCGGCTACCTGAGACTGATCC |
| | TGCCCGAGCTGCAGGCCAGAATCAGAACCTACAACCAGCACTACAACAACCTGCTGAGAGGCGCCGTGAGCCA |
| | AAGACTGTACATCCTGCTGCCCCTGGACTGCGGCGTGCCCGACAACCTTAGCATGGCCGACCCCAACATCAGAT |
| | TCCTGGACAAGCTGCCCCAGCAGACCGGCGACCACGCCGGCATCAAGGACAGAGTGTACAGCAACAGCATCTA |
| | CGAGCTGCTGGAGAACGGCCAGAGAGCCGGCACCTGCGTGCTGGAGTACGCCACCCCCCTGCAGACCCTGTTC |
| | GCCATGAGCCAGTACAGCCAGGCCGGCTTCAGCAGAGAGGACAGACTGGAGCAAGCCAAGCTGTTCTGCAGA |
| | ACCCTGGAGGACATCCTGGCGGACGCCCCCGAGAGCCAAAACAACTGCAGACTGATCGCCTACCAGGAGCCCG |
| | CCGACGACAGCAGCTTCAGCCTGAGCCAGGAAGTGCTGAGACACCTGAGACAGGAAGAGAAGGAGGAGGTG |
| | ACCGTGGGAAGCCTGAAGACCAGCGCCGTGCCCAGCACCAGCACCATGAGCCAGGAGCCCGAGCTGCTGATC |
| | AGCGGCATGGAGAAGCCCCTGCCCCTGAGAACCGACTTCAGC |
| | (huSTING(V155M); no epitope tag; nucleotide sequence) |
| 200 | ATGCCTCACAGCAGCCTGCACCCTAGCATCCCTTGCCCTAGAGGCCACGGCGCCCAGAAGGCCGCCCTGGTGCT |
| | GCTGAGCGCCTGCCTGGTGACCCTGTGGGGCCTGGGCGAGCCTCCTGAGCACACCCTGAGATACCTGGTGCTG |
| | CACCTGGCCAGCCTGCAGCTGGGCCTGCTGCTGAACGGCGTGTGCAGCCTGGCCGAGGAGCTGAGACACATCC |
| | ACAGCAGATACAGAGGCAGCTACTGGAGAACCGTGAGAGCCTGCCTGGGCTGCCCTCTGAGAAGAGGCGCCC |
| | TGCTGCTGCTGAGCATCTACTTCTACTACAGCCTGCCTAACGCCGTGGGCCCTCCTTTCACCTGGATGCTGGCCC |
| | TGCTGGGCCTGAGCCAGGCCCTGAACATCCTGCTGGGCCTGAAGGGCCTGGCCCCTGCCGAGATCAGCGCCGT |
| | GTGCGAGAAGGGCAACTTCAACGTGGCCCACGGCCTGGCCTGGAGCTACTACATCGGCTACCTGAGACTGATC |
| | CTGCCTGAGCTGCAGGCCAGAATCAGAACCTACAACCAGCACTACAACAACCTGCTGAGAGGCGCCGTGAGCC |
| | AGAGACTGTACATCCTGCTGCCTCTGGACTGCGGCGTGCCTGACAACCTGAGCATGGCCGACCCTAACATCAGA |
| | TTCCTGGACAAGCTGCCTCAGCAGACCGGCGACCACGCCGGCATCAAGGACAGAGTGTACAGCAACAGCATCT |
| | ACGAGCTGCTGGAGAACGGCCAGAGAGCCGGCACCTGCGTGCTGGAGTACGCCACCCCTCTGCAGACCCTGTT |
| | CGCCATGAGCCAGTACAGCCAGGCCGGCTTCAGCAGAGAGGACACCCTGGAGCAGGCCAAGCTGTTCTGCAG |
| | AACCCTGGAGGACATCCTGGCCGACGCCCCTGAGAGCCAGAACAACTGCAGACTGATCGCCTACCAGGAGCCT |
| | GCCGACGACAGCAGCTTCAGCCTGAGCCAGGAGGTGCTGAGACACCTGAGACAGGAGGAGAAGGAGGAGGT |
| | GACCGTGGGCAGCCTGAAGACCAGCGCCGTGCCTAGCACCAGCACCATGAGCCAGGAGCCTGAGCTGCTGAT |
| | CAGCGGCATGGAGAAGCCTCTGCCTCTGAGAACCGACTTCAGC |
| | (Hu STING(R284T); no epitope tag; nucleotide sequence) |
| 201 | ATGCCCCACAGCAGCCTGCACCCCTCCATCCCCTGTCCCAGAGGCCACGGCGCCCAGAAGGCCGCCCTGGTGCT |
| | GCTGAGCGCCTGCCTGGTGACCTTATGGGGCCTGGGCGAGCCCCCCGAGCACACCCTGAGATACCTGGTCCTG |
| | CACCTGGCCAGCCTCCAGCTGGGCCTGCTGCTCAACGGCGTGTGTAGCCTGGCCGAGGAGCTGAGACACATCC |
| | ACAGCAGATACAGAGGCAGCTACTGGAGAACCGTGAGAGCCTGCCTGGGTTGCCCACTGAGAAGAGGAGCTC |
| | TGCTGCTGCTGAGCATCTACTTCTACTACTCGCTGCCCAACGCTGTGGGCCCCCCCTTCACCTGGATGCTGGCCC |
| | TGCTGGGTCTGAGCCAGGCCCTGAACATCCTCCTGGGCCTGAAGGGCCTGGCCCCCGCCGAGATAAGCGCCGT |
| | TTGCGAGAAGGGCAACTTCAACGTGGCCCATGGCCTGGCCTGGAGCTACTACATCGGCTACTTACGCCTGATCC |
| | TGCCCGAGCTGCAGGCCAGAATCAGAACCTACAACCAGCATTACAACAACCTGCTGAGAGGCGCCGTGAGCCA |
| | GAGACTGTATATCCTGCTGCCCCTGGACTGCGGCGTGCCCGACAACCTGAGCATGGCCGACCCCAACATCAGAT |
| | TCCTGGACAAGCTCCCCCAGCAGACCGGCGACCACGCCGGAATCAAAGACAGAGTGTATAGCAACAGCATCTA |
| | CGAGCTGCTGGAGAACGGCCAGAGAGCCGGCACCTGCGTACTGGAGTACGCCACCCCCTTGCAGACCCTGTTT |
| | GCCATGAGCCAGTACAGCCAGGCCGGCTTCAGCAGAGAGGACATGCTGGAGCAGGCCAAGCTGTTCTGCAGA |
| | ACCCTGGAGGACATCCTGGCCGACGCCCCCGAGAGCCAGAACAACTGCAGACTGATCGCCTACCAAGAGCCCG |
| | CCGACGACAGCAGCTTCAGCTTAAGCCAGGAGGTGCTGAGACATCTGAGACAGGAGGAGAAGGAGGAGGTG |
| | ACCGTGGGCAGCCTCAAGACCAGCGCTGTGCCCTCTACCAGCACCATGAGCCAGGAGCCCGAGCTGCTGATCA |
| | GCGGCATGGAGAAGCCCCTGCCCCTGAGAACAGACTTCAGC |
| | (hu STING (R284M); no epitope tag; nucleotide sequence) |
| 202 | ATGCCCCATAGCAGCCTGCACCCCAGCATCCCCTGCCCCAGAGGCCACGGCGCCCAGAAGGCCGCCCTGGTCCT |
| | GCTGAGCGCATGCCTGGTCACCCTGTGGGGCCTGGGCGAGCCCCCCGAGCACACCCTGAGATACCTGGTGCTG |
| | CACCTCGCCAGCCTGCAGCTGGGCCTGCTGCTGAACGGCGTGTGCAGCCTGGCCGAGGAGCTGAGACACATCC |
| | ACAGCAGATATAGAGGCAGCTACTGGAGAACCGTGAGAGCTTGCCTCGGCTGCCCCCTGAGAAGAGGCGCCC |
| | TGCTGCTGCTGAGCATCTACTTTTACTACAGCCTGCCCAACGCTGTGGGCCCCCCTTTCACGTGGATGCTCGCCC |
| | TGCTGGGACTGAGCCAGGCCCTGAACATCCTGCTGGGCCTTAAGGGCCTAGCCCCCGCCGAGATCAGCGCCGT |
| | GTGCGAGAAGGGCAACTTCAATGTGGCCCACGGCCTGGCCTGGAGCTACTACATCGGCTACCTGAGACTGATC |
| | CTGCCCGAGCTGCAGGCCAGAATCAGAACCTACAATCAGCACTACAACAACCTGCTGAGAGGCGCCGTGAGCC |
| | AGAGACTGTACATCCTGCTGCCCCTGGACTGCGGCGTGCCCGACAACCTCAGCATGGCCGACCCCAACATCAG |
| | ATTCCTGGACAAGCTGCCCCAGCAGACCGGCGACCACGCCGGCATCAAGGATCGCGTGTACAGCAACAGCATC |
| | TACGAGCTGCTGGAAAACGGCCAGAGAGCCGGAACCTGCGTGCTGGAGTACGCCACACCCCTGCAGACCCTGT |
| | TCGCCATGAGCCAGTACAGCCAGGCCGGCTTCAGCAGAGAGGACAAGCTGGAGCAGGCCAAGCTGTTCTGCA |
| | GAACCCTGGAGGATATCCTCGCCGACGCCCCCGAGAGCCAGAACAACTGCAGGCTGATCGCGTACCAGGAGCC |
| | CGCTGACGACAGCAGCTTTAGCCTGAGCCAGGAGGTGCTGAGACATCTGCGTCAAGAGGAAAAGGAGGAGGT |
| | GACCGTGGGCTCCCTGAAGACCAGCGCCGTGCCCAGCACCAGCACCATGAGCCAGGAGCCCGAGCTGCTGATC |
| | AGCGGCATGGAGAAGCCACTGCCCCTCAGAACCGACTTCAGC |
| | (Hu STING (R284K); no epitope tag; nucleotide sequence) |
| 203 | ATGCCTCACAGCAGCCTGCACCCTAGCATCCCTTGCCCTAGAGGCCACGGCGCCCAGAAGGCCGCCCTGGTGCT |
| | GCTGAGCGCCTGCCTGGTGACCCTGTGGGGCCTGGGCGAGCCTCCTGAGCACACCCTGAGATACCTGGTGCTG |
| | CACCTGGCCAGCCTGCAGCTGGGCCTGCTGCTGAACGGCGTGTGCAGCCTGGCCGAGGAGCTGAGACACATCC |
| | ACAGCAGATACAGAGGCAGCTACTGGAGAACCGTGAGAGCCTGCCTGGGCTGCCCTCTGAGAAGAGGCGCCC |
| | TGCTGCTGCTGAGCATCTACTTCTACTACAGCCTGCCTAACGCCGTGGGCCCTCCTTTCACCTGGATGCTGGCCC |
| | TGCTGGGCCTGAGCCAGGCCCTGAACATCCTGCTGGGCCTGAAGGGCCTGGCCCCTGCCGAGATCAGCGCCGT |
| | GTGCGAGAAGGGCAACTTCAGCGTGGCCCACGGCCTGGCCTGGAGCTACTACATCGGCTACCTGAGACTGATC |
| | CTGCCTGAGCTGCAGGCCAGAATCAGAACCTACAACCAGCACTACAACAACCTGCTGAGAGGCGCCGTGAGCC |
| | AGAGACTGTACATCCTGCTGCCTCTGGACTGCGGCGTGCCTGACAACCTGAGCATGGCCGACCCTAACATCAGA |
| | TTCCTGGACAAGCTGCCTCAGCAGACCGGCGACCACGCCGGCATCAAGGACAGAGTGTACAGCAACAGCATCT |
| | ACGAGCTGCTGGAGAACGGCCAGAGAGCCGGCACCTGCGTGCTGGAGTACGCCACCCCTCTGCAGACCCTGTT |
| | CGCCATGAGCCAGTACAGCCAGGCCGGCTTCAGCAGAGAGGACAGACTGGAGCAGGCCAAGCTGTTCTGCAG |
| | AACCCTGGAGGACATCCTGGCCGACGCCCCTGAGAGCCAGAACAACTGCAGACTGATCGCCTACCAGGAGCCT |
| | GCCGACGACAGCAGCTTCAGCCTGAGCCAGGAGGTGCTGAGACACCTGAGACAGGAGGAGAAGGAGGAGGT |
| | GACCGTGGGCAGCCTGAAGACCAGCGCCGTGCCTAGCACCAGCACCATGAGCCAGGAGCCTGAGCTGCTGAT |
| | CAGCGGCATGGAGAAGCCTCTGCCTCTGAGAACCGACTTCAGC |
| | (Hu STING(N154S); no epitope tag; nucleotide sequence) |
| 204 | ATGCCTCACAGCAGCCTGCACCCTAGCATCCCTTGCCCTAGAGGCCACGGCGCCCAGAAGGCCGCCCTGGTGCT |
| | GCTGAGCGCCTGCCTGGTGACCCTGTGGGGCCTGGGCGAGCCTCCTGAGCACACCCTGAGATACCTGGTGCTG |
| | CACCTGGCCAGCCTGCAGCTGGGCCTGCTGCTGAACGGCGTGTGCAGCCTGGCCGAGGAGCTGAGACACATCC |
| | ACAGCAGATACAGAGGCAGCTACTGGAGAACCGTGAGAGCCTGCCTGGGCTGCCCTCTGAGAAGAGGCGCCC |
| | TGCTGCTGCTGAGCATCTACTTCTACTACAGCCTGCCTAACGCCGTGGGCCCTCCTTTCACCTGGATGCTGGCCC |
| | TGCTGGGCCTGAGCCAGGCCCTGAACATCCTGCTGGGCCTGAAGGGCCTGGCCCCTGCCGAGATCAGCGCCCT |
| | GTGCGAGAAGGGCAACTTCAACGTGGCCCACGGCCTGGCCTGGAGCTACTACATCGGCTACCTGAGACTGATC |
| | CTGCCTGAGCTGCAGGCCAGAATCAGAACCTACAACCAGCACTACAACAACCTGCTGAGAGGCGCCGTGAGCC |
| | AGAGACTGTACATCCTGCTGCCTCTGGACTGCGGCGTGCCTGACAACCTGAGCATGGCCGACCCTAACATCAGA |
| | TTCCTGGACAAGCTGCCTCAGCAGACCGGCGACCACGCCGGCATCAAGGACAGAGTGTACAGCAACAGCATCT |
| | ACGAGCTGCTGGAGAACGGCCAGAGAGCCGGCACCTGCGTGCTGGAGTACGCCACCCCTCTGCAGACCCTGTT |
| | CGCCATGAGCCAGTACAGCCAGGCCGGCTTCAGCAGAGAGGACAGACTGGAGCAGGCCAAGCTGTTCTGCAG |
| | AACCCTGGAGGACATCCTGGCCGACGCCCCTGAGAGCCAGAACAACTGCAGACTGATCGCCTACCAGGAGCCT |
| | GCCGACGACAGCAGCTTCAGCCTGAGCCAGGAGGTGCTGAGACACCTGAGACAGGAGGAGAAGGAGGAGGT |
| | GACCGTGGGCAGCCTGAAGACCAGCGCCGTGCCTAGCACCAGCACCATGAGCCAGGAGCCTGAGCTGCTGAT |
| | CAGCGGCATGGAGAAGCCTCTGCCTCTGAGAACCGACTTCAGC |
| | (Hu STING(V147L); no epitope tag; nucleotide sequence) |
| 205 | ATGCCTCACAGCAGCCTGCACCCTAGCATCCCTTGCCCTAGAGGCCACGGCGCCCAGAAGGCCGCCCTGGTGCT |
| | GCTGAGCGCCTGCCTGGTGACCCTGTGGGGCCTGGGCGAGCCTCCTGAGCACACCCTGAGATACCTGGTGCTG |
| | CACCTGGCCAGCCTGCAGCTGGGCCTGCTGCTGAACGGCGTGTGCAGCCTGGCCGAGGAGCTGAGACACATCC |
| | ACAGCAGATACAGAGGCAGCTACTGGAGAACCGTGAGAGCCTGCCTGGGCTGCCCTCTGAGAAGAGGCGCCC |
| | TGCTGCTGCTGAGCATCTACTTCTACTACAGCCTGCCTAACGCCGTGGGCCCTCCTTTCACCTGGATGCTGGCCC |
| | TGCTGGGCCTGAGCCAGGCCCTGAACATCCTGCTGGGCCTGAAGGGCCTGGCCCCTGCCGAGATCAGCGCCGT |
| | GTGCGAGAAGGGCAACTTCAACGTGGCCCACGGCCTGGCCTGGAGCTACTACATCGGCTACCTGAGACTGATC |
| | CTGCCTGAGCTGCAGGCCAGAATCAGAACCTACAACCAGCACTACAACAACCTGCTGAGAGGCGCCGTGAGCC |
| | AGAGACTGTACATCCTGCTGCCTCTGGACTGCGGCGTGCCTGACAACCTGAGCATGGCCGACCCTAACATCAGA |
| | TTCCTGGACAAGCTGCCTCAGCAGACCGGCGACCACGCCGGCATCAAGGACAGAGTGTACAGCAACAGCATCT |
| | ACGAGCTGCTGGAGAACGGCCAGAGAGCCGGCACCTGCGTGCTGGAGTACGCCACCCCTCTGCAGACCCTGTT |
| | CGCCATGAGCCAGTACAGCCAGGCCGGCTTCAGCAGAGAGGACAGACTGGAGCAGGCCAAGCTGTTCTGCAG |
| | AACCCTGGAGGACATCCTGGCCGACGCCCCTGAGAGCCAGAACAACTGCAGACTGATCGCCTACCAGCAGCCT |
| | GCCGACGACAGCAGCTTCAGCCTGAGCCAGGAGGTGCTGAGACACCTGAGACAGGAGGAGAAGGAGGAGGT |
| | GACCGTGGGCAGCCTGAAGACCAGCGCCGTGCCTAGCACCAGCACCATGAGCCAGGAGCCTGAGCTGCTGAT |
| | CAGCGGCATGGAGAAGCCTCTGCCTCTGAGAACCGACTTCAGC |
| | (Hu STING (E315Q); no epitope tag; nucleotide sequence) |
| 206 | ATGCCTCACAGCAGCCTGCACCCTAGCATCCCTTGCCCTAGAGGCCACGGCGCCCAGAAGGCCGCCCTGGTGCT |
| | GCTGAGCGCCTGCCTGGTGACCCTGTGGGGCCTGGGCGAGCCTCCTGAGCACACCCTGAGATACCTGGTGCTG |
| | CACCTGGCCAGCCTGCAGCTGGGCCTGCTGCTGAACGGCGTGTGCAGCCTGGCCGAGGAGCTGAGACACATCC |
| | ACAGCAGATACAGAGGCAGCTACTGGAGAACCGTGAGAGCCTGCCTGGGCTGCCCTCTGAGAAGAGGCGCCC |
| | TGCTGCTGCTGAGCATCTACTTCTACTACAGCCTGCCTAACGCCGTGGGCCCTCCTTTCACCTGGATGCTGGCCC |
| | TGCTGGGCCTGAGCCAGGCCCTGAACATCCTGCTGGGCCTGAAGGGCCTGGCCCCTGCCGAGATCAGCGCCGT |
| | GTGCGAGAAGGGCAACTTCAACGTGGCCCACGGCCTGGCCTGGAGCTACTACATCGGCTACCTGAGACTGATC |
| | CTGCCTGAGCTGCAGGCCAGAATCAGAACCTACAACCAGCACTACAACAACCTGCTGAGAGGCGCCGTGAGCC |
| | AGAGACTGTACATCCTGCTGCCTCTGGACTGCGGCGTGCCTGACAACCTGAGCATGGCCGACCCTAACATCAGA |
| | TTCCTGGACAAGCTGCCTCAGCAGACCGGCGACCACGCCGGCATCAAGGACAGAGTGTACAGCAACAGCATCT |
| | ACGAGCTGCTGGAGAACGGCCAGAGAGCCGGCACCTGCGTGCTGGAGTACGCCACCCCTCTGCAGACCCTGTT |
| | CGCCATGAGCCAGTACAGCCAGGCCGGCTTCAGCAGAGAGGACAGACTGGAGCAGGCCAAGCTGTTCTGCAG |
| | AACCCTGGAGGACATCCTGGCCGACGCCCCTGAGAGCCAGAACAACTGCAGACTGATCGCCTACCAGGAGCCT |
| | GCCGACGACAGCAGCTTCAGCCTGAGCCAGGAGGTGCTGAGACACCTGAGACAGGAGGAGAAGGAGGAGGT |
| | GACCGTGGGCAGCCTGAAGACCAGCGCCGTGCCTAGCACCAGCACCATGAGCCAGGAGCCTGAGCTGCTGAT |
| | CAGCGGCATGGAGAAGCCTCTGCCTCTGGCCACCGACTTCAGC |
| | (Hu STING (R375A); no epitope tag; nucleotide sequence) |
| 207 | ATGCCTCACAGCAGCCTGCACCCTAGCATCCCTTGCCCTAGAGGCCACGGCGCCCAGAAGGCCGCCCTGGTGCT |
| | GCTGAGCGCCTGCCTGGTGACCCTGTGGGGCCTGGGCGAGCCTCCTGAGCACACCCTGAGATACCTGGTGCTG |
| | CACCTGGCCAGCCTGCAGCTGGGCCTGCTGCTGAACGGCGTGTGCAGCCTGGCCGAGGAGCTGAGACACATCC |
| | ACAGCAGATACAGAGGCAGCTACTGGAGAACCGTGAGAGCCTGCCTGGGCTGCCCTCTGAGAAGAGGCGCCC |
| | TGCTGCTGCTGAGCATCTACTTCTACTACAGCCTGCCTAACGCCGTGGGCCCTCCTTTCACCTGGATGCTGGCCC |
| | TGCTGGGCCTGAGCCAGGCCCTGAACATCCTGCTGGGCCTGAAGGGCCTGGCCCCTGCCGAGATCAGCGCCCT |
| | GTGCGAGAAGGGCAACTTCAGCATGGCCCACGGCCTGGCCTGGAGCTACTACATCGGCTACCTGAGACTGATC |
| | CTGCCTGAGCTGCAGGCCAGAATCAGAACCTACAACCAGCACTACAACAACCTGCTGAGAGGCGCCGTGAGCC |
| | AGAGACTGTACATCCTGCTGCCTCTGGACTGCGGCGTGCCTGACAACCTGAGCATGGCCGACCCTAACATCAGA |
| | TTCCTGGACAAGCTGCCTCAGCAGACCGGCGACCACGCCGGCATCAAGGACAGAGTGTACAGCAACAGCATCT |
| | ACGAGCTGCTGGAGAACGGCCAGAGAGCCGGCACCTGCGTGCTGGAGTACGCCACCCCTCTGCAGACCCTGTT |
| | CGCCATGAGCCAGTACAGCCAGGCCGGCTTCAGCAGAGAGGACAGACTGGAGCAGGCCAAGCTGTTCTGCAG |
| | AACCCTGGAGGACATCCTGGCCGACGCCCCTGAGAGCCAGAACAACTGCAGACTGATCGCCTACCAGGAGCCT |
| | GCCGACGACAGCAGCTTCAGCCTGAGCCAGGAGGTGCTGAGACACCTGAGACAGGAGGAGAAGGAGGAGGT |
| | GACCGTGGGCAGCCTGAAGACCAGCGCCGTGCCTAGCACCAGCACCATGAGCCAGGAGCCTGAGCTGCTGAT |
| | CAGCGGCATGGAGAAGCCTCTGCCTCTGAGAACCGACTTCAGC |
| | (Hu STING(V147L/N154S/V155M); no epitope tag; nucleotide sequence) |
| 208 | ATGCCTCACAGCAGCCTGCACCCTAGCATCCCTTGCCCTAGAGGCCACGGCGCCCAGAAGGCCGCCCTGGTGCT |
| | GCTGAGCGCCTGCCTGGTGACCCTGTGGGGCCTGGGCGAGCCTCCTGAGCACACCCTGAGATACCTGGTGCTG |
| | CACCTGGCCAGCCTGCAGCTGGGCCTGCTGCTGAACGGCGTGTGCAGCCTGGCCGAGGAGCTGAGACACATCC |
| | ACAGCAGATACAGAGGCAGCTACTGGAGAACCGTGAGAGCCTGCCTGGGCTGCCCTCTGAGAAGAGGCGCCC |
| | TGCTGCTGCTGAGCATCTACTTCTACTACAGCCTGCCTAACGCCGTGGGCCCTCCTTTCACCTGGATGCTGGCCC |
| | TGCTGGGCCTGAGCCAGGCCCTGAACATCCTGCTGGGCCTGAAGGGCCTGGCCCCTGCCGAGATCAGCGCCCT |
| | GTGCGAGAAGGGCAACTTCAGCATGGCCCACGGCCTGGCCTGGAGCTACTACATCGGCTACCTGAGACTGATC |
| | CTGCCTGAGCTGCAGGCCAGAATCAGAACCTACAACCAGCACTACAACAACCTGCTGAGAGGCGCCGTGAGCC |
| | AGAGACTGTACATCCTGCTGCCTCTGGACTGCGGCGTGCCTGACAACCTGAGCATGGCCGACCCTAACATCAGA |
| | TTCCTGGACAAGCTGCCTCAGCAGACCGGCGACCACGCCGGCATCAAGGACAGAGTGTACAGCAACAGCATCT |
| | ACGAGCTGCTGGAGAACGGCCAGAGAGCCGGCACCTGCGTGCTGGAGTACGCCACCCCTCTGCAGACCCTGTT |
| | CGCCATGAGCCAGTACAGCCAGGCCGGCTTCAGCAGAGAGGACATGCTGGAGCAGGCCAAGCTGTTCTGCAG |
| | AACCCTGGAGGACATCCTGGCCGACGCCCCTGAGAGCCAGAACAACTGCAGACTGATCGCCTACCAGGAGCCT |
| | GCCGACGACAGCAGCTTCAGCCTGAGCCAGGAGGTGCTGAGACACCTGAGACAGGAGGAGAAGGAGGAGGT |
| | GACCGTGGGCAGCCTGAAGACCAGCGCCGTGCCTAGCACCAGCACCATGAGCCAGGAGCCTGAGCTGCTGAT |
| | CAGCGGCATGGAGAAGCCTCTGCCTCTGAGAACCGACTTCAGC |
| | (Hu STING(R284M/V147L/N154S/V155M); no epitope tag; nucleotide sequence) |
| 209 | TGATAATAGGCTGGAGCCTCGGTGGCCTAGCTTCTTGCCCCTTGGGCCTCCCCCCAGCCCCTCCTCCCCTTCCTG |
| | CACCCGTACCCCCCAAACACCATTGTCACACTCCAGTGGTCTTTGAATAAAGTCTGAGTGGGCGGC |
| | (3′ UTR used in STING V155M construct, containing miR122 binding site) |
| 210 | ATGGAGACCCCCAAGCCTAGAATCCTGCCCTGGCTGGTGAGCCAGCTGGACCTGGGCCAGCTGGAGGGCGTA |
| | GCCTGGCTGGACGAGAGCAGAACCAGATTCAGAATCCCCTGGAAGCACGGCCTGAGACAAGACGCCCAGATG |
| | GCCGACTTCGGCATCTTCCAGGCCTGGGCCGAGGCCAGCGGCGCCTACACCCCTGGCAAGGATAAGCCCGATG |
| | TGAGCACCTGGAAGAGAAACTTCAGAAGCGCCCTGAACAGAAAGGAGGTGCTGAGACTGGCCGCCGACAATA |
| | GCAAGGACCCCTACGACCCCCACAAGGTGTACGAGTTCGTTACCCCCGGCGCCAGGGACTTCGTGCACCTGGG |
| | CGCCAGCCCCGACACCAACGGCAAGAGCAGCCTGCCCCACAGCCAGGAGAACCTGCCCAAGCTGTTCGATGGC |
| | CTGATCCTGGGCCCCCTGAAGGACGAGGGCAGCAGCGACCTGGCCATCGTGAGCGACCCTAGCCAGCAGCTG |
| | CCCTCCCCCAACGTGAACAACTTCCTGAACCCCGCCCCCCAGGAGAACCCCCTGAAGCAACTGCTGGCCGAGGA |
| | GCAGTGGGAGTTCGAGGTGACCGCCTTCTACAGAGGCAGACAGGTGTTCCAGCAGACCCTGTTCTGCCCCGGC |
| | GGCCTGAGACTGGTAGGCAGCACCGCTGACATGACCCTGCCCTGGCAGCCCGTGACCCTGCCCGACCCCGAAG |
| | GCTTTCTGACCGACAAGCTGGTGAAGGAGTACGTCGGCCAAGTGCTGAAGGGCCTGGGCAACGGCCTGGCCC |
| | TGTGGCAGGCCGGCCAGTGCCTGTGGGCCCAGAGACTCGGCCACAGCCACGCCTTCTGGGCCCTGGGCGAGG |
| | AACTCCTGCCCGATAGCGGCAGAGGCCCCGACGGCGAGGTGCACAAGGACAAGGACGGCGCCGTGTTCGACC |
| | TGCGCCCCTTCGTGGCCGACCTGATCGCCTTCATGGAGGGCAGCGGCCACAGCCCCAGATATACCCTGTGGTTC |
| | TGCATGGGCGAGATGTGGCCCCAGGACCAGCCCTGGGTGAAGAGACTGGTGATGGTGAAGGTGGTGCCCACC |
| | TGCCTGAAAGAGCTGCTGGAGATGGCCAGAGAGGGCGGCGCCAGCTCCCTGAAAACCGTGGACCTGCACATT |
| | GACAACAGCCAGCCCATCAGCCTGACCAGCGACCAGTACAAGGCCTACCTGCAGGACCTGGTGGAGGACATG |
| | GACTTCCAGGCCACCGGCAACATC |
| | (super mouse IRF3 S396D; no epitope tag) |
| 211 | ATGGGCACCCCCAAGCCCAGAATCCTGCCCTGGCTGGTGAGCCAGCTGGACCTGGGCCAGCTGGAGGGAGTG |
| | GCCTGGGTGAACAAGAGCAGAACCAGATTCAGAATCCCCTGGAAGCACGGCCTCAGACAGGACGCCCAGCAG |
| | GAGGACTTCGGCATTTTTCAGGCTTGGGCCGAGGCCACCGGCGCCTACGTGCCCGGCAGAGACAAGCCCGACC |
| | TGCCCACCTGGAAAAGAAACTTCAGAAGCGCCTTGAATAGAAAGGAGGGCCTGAGACTGGCCGAGGACAGAA |
| | GCAAGGACCCCCACGACCCTCACAAGATCTACGAGTTCGTGAATAGCGGCGTGGGCGACTTTAGCCAGCCCGA |
| | CACCAGCCCCGACACCAACGGCGGCGGCAGCACCAGCGACACGCAGGAGGACATCCTGGATGAACTGCTGGG |
| | CAACATGGTGCTGGCCCCCCTGCCCGATCCCGGCCCCCCTTCGCTTGCCGTGGCCCCCGAGCCCTGCCCCCAGC |
| | CCCTGAGAAGCCCCTCTCTGGATAACCCCACCCCCTTCCCCAACCTGGGCCCCAGCGAGAATCCACTGAAGAGA |
| | CTTCTGGTCCCCGGCGAGGAGTGGGAGTTCGAGGTGACCGCCTTCTACAGAGGCAGACAGGTGTTCCAGCAG |
| | ACCATCAGCTGCCCCGAAGGCCTGAGATTAGTGGGCAGCGAAGTGGGCGACAGGACCCTGCCCGGGTGGCCC |
| | GTGACCCTGCCCGATCCCGGCATGAGCCTGACCGACAGAGGTGTGATGAGCTACGTGAGACACGTGCTGAGCT |
| | GCCTGGGCGGCGGCCTGGCACTGTGGAGAGCCGGCCAGTGGCTGTGGGCCCAGAGACTGGGCCACTGCCACA |
| | CCTACTGGGCCGTGAGCGAGGAGCTGCTGCCCAACAGCGGCCACGGCCCCGACGGCGAGGTGCCCAAGGACA |
| | AGGAAGGGGGCGTGTTCGACCTGGGCCCCTTCATCGTAGACCTGATCACCTTTACCGAGGGCAGCGGCAGGA |
| | GCCCCAGATACGCCCTGTGGTTCTGCGTGGGCGAAAGCTGGCCCCAGGACCAGCCCTGGACCAAGAGACTGGT |
| | GATGGTGAAGGTAGTGCCCACCTGCCTGAGAGCCTTAGTGGAGATGGCCAGAGTGGGCGGGGCCAGCAGCCT |
| | GGAGAACACCGTGGATCTTCACATCGACAACAGCCACCCCCTGAGCCTGACCAGCGACCAGTACAAGGCCTAC |
| | CTGCAGGACCTGGTGGAGGGCATGGACTTCCAGGGCCCCGGCGAGACC |
| | (super human IRF3 S396D; no epitope tag) |
| 212 | ATGGCGCTGGCCCCCGAAAGAGCCGCCCCCAGAGTCCTCTTCGGCGAATGGCTCCTTGGCGAAATTTCGTCGG |
| | GCTGCTACGAGGGCTTACAATGGCTGGATGAGGCGAGAACCTGTTTCAGGGTGCCCTGGAAACACTTCGCCAG |
| | AAAGGATCTAAGCGAAGCAGATGCTAGAATTTTTAAGGCTTGGGCCGTGGCCAGGGGAAGATGGCCCCCCTC |
| | GAGCAGAGGCGGCGGCCCTCCCCCCGAGGCAGAAACGGCCGAGAGAGCCGGATGGAAAACCAATTTCAGAT |
| | GCGCCCTGAGATCTACAAGAAGATTCGTGATGCTTAGAGACAACAGCGGAGATCCCGCCGATCCCCATAAGGT |
| | GTATGCCCTGTCCCGGGAGCTGTGCTGGAGGGAAGGGCCTGGCACTGACCAGACCGAAGCCGAAGCCCCCGC |
| | GGCCGTGCCGCCGCCCCAAGGAGGCCCACCAGGCCCTTTCCTCGCTCACACCCACGCCGGTCTGCAAGCCCCG |
| | GGACCTCTACCTGCCCCTGCCGGCGATAAAGGCGACCTGTTGCTGCAGGCCGTCCAACAGAGCTGCCTGGCCG |
| | ATCATCTGCTCACAGCCAGCTGGGGCGCTGACCCCGTCCCAACAAAGGCCCCCGGTGAGGGCCAAGAAGGCCT |
| | GCCTCTGACCGGCGCCTGTGCCGGCGGCCCTGGCCTGCCTGCTGGCGAGCTGTACGGATGGGCTGTCGAAACC |
| | ACTCCCTCCCCCGGCCCCCAACCTGCGGCCCTGACAACCGGCGAGGCAGCCGCACCCGAAAGCCCCCACCAGG |
| | CCGAACCCTACCTCAGTCCCAGCCCCTCCGCCTGCACCGCTGTGCAGGAGCCCAGCCCCGGTGCTCTGGACGTA |
| | ACAATCATGTACAAAGGCAGAACCGTGCTTCAGAAGGTGGTTGGACACCCCTCCTGTACTTTTCTCTACGGCCC |
| | CCCCGACCCTGCCGTGAGAGCTACCGACCCGCAACAGGTGGCCTTTCCCTCGCCCGCCGAACTGCCCGATCAAA |
| | AACAGCTGAGATACACCGAGGAGCTGCTGAGACACGTGGCGCCGGGCTTACACCTAGAGTTGAGAGGCCCCC |
| | AACTCTGGGCCAGACGCATGGGCAAGTGTAAGGTGTACTGGGAGGTCGGGGGCCCTCCCGGCTCTGCCAGCC |
| | CCAGCACCCCTGCTTGTCTCTTGCCCAGAAACTGTGATACCCCCATCTTCGACTTCCGTGTATTTTTCCAGGAACT |
| | GGTCGAGTTTAGAGCCAGACAGAGACGAGGCAGCCCCAGATATACAATCTACCTCGGCTTCGGCCAGGACCTG |
| | AGTGCCGGCAGACCTAAGGAGAAGTCGCTGGTCCTAGTGAAGTTAGAGCCCTGGCTATGTAGAGTGCACCTG |
| | GAGGGCACCCAGAGAGAAGGAGTGAGCAGCCTGGACAGCAGCAGCCTGAGTCTGTGCCTGAGCTCCGCCAAC |
| | TCGCTGTATGATGACATCGAGTGTTTCCTCATGGAGCTGGAGCAGCCCGCC |
| | (Wild-type Hu IRF7 isoform A; P037 without epitope tag) |
| 213 | ATGGCCCTTGCCCCTGAGCGGGCCGCCCCCAGAGTGTTATTCGGCGAGTGGCTGCTGGGCGAGATCAGCAGCG |
| | GCTGCTACGAGGGACTGCAGTGGCTGGACGAGGCTAGAACCTGCTTCAGAGTGCCCTGGAAGCATTTCGCCAG |
| | AAAAGACCTGAGCGAGGCTGATGCTAGAATCTTCAAAGCCTGGGCTGTGGCCCGAGGAAGATGGCCCCCCAG |
| | CAGCAGAGGAGGCGGCCCTCCTCCCGAGGCCGAAACCGCAGAGCGTGCTGGCTGGAAAACCAACTTTAGGTG |
| | TGCCCTGAGGAGCACCAGAAGATTCGTTATGCTCAGAGACAACAGCGGGGACCCCGCCGACCCGCACAAGGT |
| | GTACGCCTTAAGTAGGGAGCTGTGCTGGAGAGAGGGACCGGGGACCGACCAAACCGAGGCTGAGGCGCCCG |
| | CCGCCGTTCCACCTCCCCAGGGTGGTCCCCCAGGGCCCTTTCTGGCACACACCCACGCCGGATTACAGGCGCCA |
| | GGGCCCTTACCCGCCCCCGCCGGAGACAAAGGCGACCTCCTGCTGCAAGCCGTGCAACAAAGCTGCCTGGCCG |
| | ATCACTTACTAACCGCTAGCTGGGGCGCCGATCCTGTTCCCACCAAGGCCCCCGGTGAAGGGCAAGAAGGACT |
| | GCCCTTAACCGGCGCCTGTGCCGGAGGCCCTGGTCTGCCAGCCGGCGAGCTGTACGGTTGGGCTGTCGAAACA |
| | ACACCCAGTCCGGGCCCACAGCCTGCCGCTCTGACCACCGGCGAAGCCGCCGCCCCCGAGAGCCCACACCAGG |
| | CTGAACCCTACCTGAGCCCCAGCCCCAGCGCCTGCACCGCTGTGCAGGAGCCTAGCCCCGGCGCTCTTGATGTG |
| | ACAATAATGTACAAGGGCAGGACCGTGCTGCAAAAGGTCGTGGGCCATCCGTCGTGTACCTTTCTGTACGGCC |
| | CTCCAGACCCCGCGGTTAGAGCCACCGACCCCCAGCAAGTCGCCTTCCCCTCCCCCGCCGAACTGCCCGACCAA |
| | AAGCAGCTGCGGTACACAGAAGAACTACTTAGACACGTGGCCCCCGGTCTGCACTTGGAGCTGAGAGGCCCCC |
| | AGCTCTGGGCCAGAAGAATGGGCAAGTGCAAAGTGTACTGGGAGGTGGGCGGCCCACCCGGCTCAGCTTCGC |
| | CCTCCACACCCGCATGCCTGCTGCCCAGAAATTGCGACACGCCCATCTTCGATTTTAGAGTGTTCTTTCAGGAGT |
| | TGGTGGAGTTCAGAGCCAGACAAAGACGCGGCAGCCCCAGATACACCATTTACCTCGGCTTCGGCCAGGACCT |
| | CAGCGCTGGCAGACCCAAGGAGAAGAGTCTGGTCCTCGTGAAGCTGGAGCCCTGGCTGTGCAGAGTGCACCT |
| | GGAGGGCACCCAGCGTGAAGGCGTGAGCAGCCTGGATTCAAGCGACCTGGACCTATGCCTAAGCAGCGCTAA |
| | CTCACTGTACGACGATATCGAATGCTTCCTGATGGAACTGGAGCAGCCTGCC |
| | (constitutively active Hu IRF7 S477D/S479D; P033 without epitope tag) |
| 214 | ATGGCCCTGGCACCCGAGAGGGCCGCCCCCAGGGTGCTCTTCGGCGAGTGGTTACTAGGCGAAATTAGCAGC |
| | GGCTGCTATGAAGGCCTTCAGTGGCTGGACGAGGCCAGAACCTGCTTTAGAGTTCCCTGGAAGCACTTCGCCC |
| | GGAAAGATCTCTCTGAAGCCGACGCCAGAATATTCAAGGCCTGGGCTGTCGCCAGGGGCAGGTGGCCACCCTC |
| | CAGCCGAGGTGGCGGCCCTCCCCCTGAGGCTGAGACTGCGGAAAGGGCGGGCTGGAAGACCAATTTCAGATG |
| | CGCTCTGAGAAGCACCAGACGTTTTGTGATGCTAAGAGACAATAGCGGCGATCCCGCCGACCCCCATAAGGTA |
| | TACGCACTGAGCCGAGAGCTCTGTTGGAGAGAAGGCCCCGGCACCGACCAGACCGAGGCTGAAGCCCCTGCA |
| | GCCGTGCCCCCCCCTCAAGGCGGGCCCCCCGGCCCCTTCCTGGCCCATACCCATGCAGGGTTACAAGCACCCGG |
| | GCCCTTGCCCGCCCCAGCGGGAGACAAGGGCGACCTCTTACTGCAGGCCGTGCAACAAAGTTGTCTGGCGGAC |
| | CACCTGCTGACCGCATCATGGGGCGCGGATCCTGTGCCCACCAAGGCACCCGGCGAAGGCCAGGAGGGCCTG |
| | CCCTTGACCGGCGCCTGCGCTGGCGGACCCGGCCTACCTGCTGGCGAACTGTATGGCTGGGCCGTAGAGACGA |
| | CTCCCAGCCCTGGCCCACAACCCGCGGCTTTGACCACCGGCGAAGCCGCCGCCCCCGAGTCTCCGCACCAGGCC |
| | GAGCCTTACCTCAGCCCAAGCCCTAGCGCCTGCACCGCCGTGCAAGAACCTAGCCCCGGAGCCCTGGATGTGA |
| | CAATCATGTACAAGGGTAGAACCGTACTGCAAAAGGTGGTGGGTCATCCCAGCTGCACCTTTCTTTACGGCCCA |
| | CCCGACCCTGCCGTGCGAGCCACAGACCCACAACAGGTCGCCTTCCCAAGCCCCGCCGAACTGCCCGATCAGA |
| | AACAGCTGAGATATACAGAGGAGCTTCTGCGGCACGTAGCTCCCGGCCTACATCTCGAGCTGAGGGGCCCACA |
| | ACTGTGGGCCAGACGCATGGGCAAATGCAAGGTCTACTGGGAAGTGGGAGGCCCCCCCGGCAGCGCATCTCC |
| | CAGCACGCCCGCGTGCCTGCTGCCTAGAAATTGCGACACCCCCATCTTTGACTTCCGGGTATTCTTTCAGGAGCT |
| | GGTAGAGTTCAGAGCCAGGCAGCGGAGGGGCTCCCCCAGATACACAATCTACCTGGGCTTCGGACAGGACCT |
| | GTCCGCCGGCCGCCCCAAGGAAAAGAGCCTGGTGCTGGTGAAGCTGGAGCCCTGGCTGTGTAGGGTACACCT |
| | CGAAGGCACCCAGAGAGAAGGAGTGAGCTCGCTTGATGACAGCGATCTGTCGGATTGCCTTAGCAGCGCCAA |
| | CAGCCTGTATGATGATATCGAGTGCTTCCTTATGGAACTGGAGCAGCCCGCC |
| | (constitutively active Hu IRF7 S475D/S477D/L480D; P034 without epitope tag) |
| 215 | ATGGCCCTAGCCCCCGAAAGAGCAGCTCCCAGAGTGCTGTTCGGCGAATGGCTGCTTGGCGAGATCAGCAGCG |
| | GCTGCTACGAAGGCCTGCAGTGGCTGGACGAAGCCCGCACCTGTTTCAGAGTGCCCTGGAAGCACTTCGCTAG |
| | AAAGGATTTGAGCGAGGCTGATGCTAGAATCTTTAAGGCTTGGGCTGTGGCAAGAGGCAGATGGCCGCCTAG |
| | TAGCAGAGGGGGCGGACCTCCCCCCGAGGCTGAGACCGCTGAGAGAGCAGGGTGGAAAACCAACTTCAGATG |
| | CGCGCTGAGAAGCACCCGAAGATTCGTGATGCTACGTGACAATAGCGGCGACCCCGCCGACCCCCACAAAGTG |
| | TACGCCCTGTCCCGAGAACTTTGCTGGAGAGAGGGACCCGGCACCGATCAAACAGAGGCTGAGGCCCCGGCC |
| | GCTGTACCCCCGCCCCAAGGAGGCCCCCCAGGCCCCTTTCTGGCTCATACACATGCCGGCCTGCAGGCACCCGG |
| | GCCCCTCCCGGCTCCTGCCGGCGACAAGGGCGATCTCCTTCTCCAGGCCGTGCAGCAGAGCTGCCTGGCCGAT |
| | CACCTGCTGACCGCCTCGTGGGGCGCCGACCCCGTGCCCACCAAAGCCCCGGGTGAAGGCCAAGAGGGGCTC |
| | CCTTTAACCGGAGCATGCGCCGGAGGCCCCGGCCTGCCAGCCGGCGAGTTATATGGCTGGGCTGTGGAGACC |
| | ACACCCTCCCCCGGCCCTCAACCCGCTGCCCTGACCACCGGTGAGGCCGCCGCCCCCGAGAGCCCACACCAGGC |
| | CGAACCCTACCTGAGCCCTAGCCCTAGCGCCTGCACCGCCGTGCAAGAACCCAGCCCCGGAGCCCTGGATGTG |
| | ACCATTATGTACAAGGGCCGGACAGTGCTGCAAAAGGTTGTGGGACACCCGAGCTGCACCTTTCTGTACGGTC |
| | CGCCTGACCCCGCCGTGAGAGCCACGGACCCGCAGCAGGTGGCCTTCCCCTCACCCGCGGAGCTGCCCGACCA |
| | AAAGCAACTCAGATACACAGAAGAACTATTGCGTCACGTCGCGCCCGGCCTGCATCTGGAGCTGAGAGGCCCC |
| | CAGCTCTGGGCCAGAAGGATGGGCAAATGCAAGGTGTACTGGGAGGTGGGAGGCCCCCCCGGCAGCGCCAG |
| | CCCCAGCACTCCCGCGTGCCTGCTGCCCAGAAATTGCGACACTCCCATCTTCGATTTCAGGGTGTTCTTCCAGGA |
| | GCTGGTGGAGTTCAGAGCCAGGCAGAGAAGGGGTAGCCCCAGATACACAATCTATCTAGGCTTTGGACAAGA |
| | TCTGAGCGCCGGCCGGCCTAAGGAAAAAAGCCTGGTGCTGGTAAAGCTGGAGCCGTGGCTTTGTAGAGTGCA |
| | CCTGGAGGGGACGCAGCGAGAGGGCGTGAGCAGCTTAGACGACGATGACTTGGATCTGTGTCTCGACAGCGC |
| | CAACGACTTGTACGACGACATCGAGTGCTTCCTGATGGAACTGGAGCAGCCCGCC |
| | (constitutively active Hu IRF7 S475D/S476D/S477D/S479D/S483D/S487D; P035 without |
| | epitope tag) |
| 216 | ATGGCCCTGGCCCCCGAGAGAGCCGCCCCCAGAGTGCTCTTCGGCGAGTGGCTGCTGGGCGAGATAAGCAGC |
| | GGCTGCTACGAAGGTCTGCAGTGGCTAGACGAGGCCAGAACCTGCTTTAGAGTGCCCTGGAAGCACTTCGCTC |
| | GAAAGGACCTGTCCGAGGCCGATGCTAGAATTTTTAAGGCTTGGGCCGTCGCTAGGGGAAGATGGCCCCCTAG |
| | CAGTAGAGGCGGCGGCCCCCCTCCCGAAGCCGAGACGGCCGAGAGGGCCGGCTGGAAAACCAATTTCAGATG |
| | CGCCCTGAGGAGCACCCGCAGGTTCGTAATGCTGCGAGACAATAGCGGCGATCCTGCGGATCCTCACAAGGTT |
| | TACGCCTTGAGTAGAGAACTGTGCTGGCGGGAGGGCCCCGGAACCGACCAGACGGAGGCAGAGGCACCCGCT |
| | GCCGTGCCCCCCCCTCAAGGAGGACCCCCTGGACCCTTTCTGGCCCACACCCACGCTGGTCTGCAGGCCCCAGG |
| | CCCACTGCCCGCCCCAGCGGGCGATAAGGGTGACCTGCTCCTACAGGCGGTGCAACAGAGCTGTCTGGCCGAC |
| | CACCTGTTGACCGCCAGCTGGGGGGCCGACCCGGTGCCCACCAAAGCTCCCGGAGAGGGCCAAGAAGGCCTC |
| | CCACTAACTGGCGCCTGCGCCGGGGGCCCGGGATTACCCGCCGGCGAGCTGTATGGCTGGGCCGTGGAGACC |
| | ACGCCCAGCCCCGAGGGCGTGTCGTCCCTGGACAGCAGCAGCCTGAGCCTGTGCCTGAGCTCCGCCAACAGCC |
| | TGTATGACGACATCGAGTGCTTCCTGATGGAGCTGGAACAACCCGCC |
| | (constitutively active truncated Hu IRF7 1-246 + 468-503; P032 without epitope tag) |
| 217 | ATGGCACTGGCGCCTGAAAGAGCCGCTCCGCGTGTGCTCTTCGGCGAGTGGCTGCTGGGCGAGATCAGCTCCG |
| | GCTGCTACGAGGGTCTACAGTGGCTGGACGAGGCCAGAACCTGTTTTAGAGTGCCCTGGAAGCACTTCGCGAG |
| | AAAGGACCTGAGCGAGGCCGACGCCAGAATCTTCAAAGCCTGGGCAGTGGCTAGGGGCAGATGGCCTCCCAG |
| | CAGCCGGGGCGGCGGCCCACCCCCCGAGGCCGAAACCGCCGAAAGAGCTGGCTGGAAGACCAACTTCAGATG |
| | CGCCCTGAGAAGCACCAGAAGATTTGTCATGCTGAGAGATAATTCAGGAGACCCCGCCGACCCTCACAAGGTG |
| | TACGCCCTGTCCAGAGAGCTGTGTTGGAGAGAGGGCCCCGGAACCGACCAGACCGAGGCCGAGGCTCCAGCT |
| | GCCGTGCCACCCCCCCAAGGCGGACCACCCGGCCCCTTCTTGGCACATACGCACGCCGGCCTCCAGGCTCCCGG |
| | CCCTCTGCCCGCCCCTGCTGGTGACAAAGGCGATCTGCTGCTGCAAGCCGTCCAGCAATCCTGCTTGGCTGACC |
| | ACCTGCTGACCGCTAGCTGGGGAGCCGACCCCGTTCCCACCAAGGCTCCCGGAGAAGGACAGGAGGGCCTGC |
| | CCCTTACCGGCGCTTGCGCGGGGGGCCCTGGCTTGCCTGCCGGCGAACTGTACGGCTGGGCCGTGGAGACCAC |
| | GCCTTCCCCCGAGGGCGTGTCCAGCCTGGACGATGATGACCTGGATCTGTGCCTGGACAGCGCCAACGACCTG |
| | TACGATGACATCGAGTGCTTTTTGATGGAGCTGGAGCAGCCCGCC |
| | (constitutively active truncated Hu IRF7 1-246 + 468-503 plus |
| | S475D/S476D/S477D/S479D/S483D/S487D; P036 without epitope tag) |
| 218 | ATGGCCCTGGCCCCCGAGAGAGCCGCGCCCAGAGTGCTGTTCGGCGAATGGCTGCTGGGCGAGATCAGCAGC |
| | GGCTGCTATGAGGGCCTGCAGTGGCTCGACGAAGCCAGGACGTGCTTCAGAGTCCCCTGGAAGCACTTCGCCA |
| | GAAAGGATCTGAGCGAGGCTGACGCCAGAATCTTCAAGGCCTGGGCAGTTGCGCGTGGGAGATGGCCCCCCA |
| | GCTCGCGGGGCGGCGGTCCCCCCCCTGAGGCCGAGACCGCCGAAAGAGCCGGATGGAAAACCAACTTTCGAT |
| | GCGCCCTCAGAAGCACCAGACGGTTTGTGATGCTGAGAGATAACAGCGGCGACCCTGCAGACCCCCATAAAGT |
| | GTATGCCCTGAGCAGAGAGCTGTGTTGGCGAGAGGGCCCCGGAACCGACCAAACCGAGGCCGAGGCCCCCGC |
| | CGCCGTACCCCCCCCTCAAGGCCCCCAGCCTGCTGCTCTGACCACGGGAGAAGCCGCCGCTCCTGAGAGCCCCC |
| | ACCAAGCCGAGCCCTATCTGAGCCCTAGCCCCAGCGCCTGCACCGCCGTGCAGGAGCCCTCACCGGGCGCCCT |
| | AGACGTGACCATCATGTACAAGGGGCGCACGGTGCTGCAAAAGGTGGTGGGCCACCCCAGCTGCACCTTCCTG |
| | TACGGCCCCCCCGACCCTGCCGTGAGAGCCACCGACCCCCAGCAAGTCGCCTTCCCCAGCCCCGCCGAGCTGCC |
| | CGACCAGAAGCAGCTGAGGTACACCGAGGAGTTGCTGAGACATGTGGCCCCCGGCTTGCACCTCGAGCTGAG |
| | AGGCCCGCAGCTCTGGGCCAGAAGAATGGGCAAGTGCAAGGTGTACTGGGAGGTGGGCGGCCCCCCCGGCA |
| | GCGCGAGCCCAAGCACCCCGGCCTGCCTGCTGCCTAGAAACTGCGACACCCCTATCTTCGACTTCAGAGTATTT |
| | TTCCAGGAGCTGGTCGAGTTCAGGGCCAGACAGCGTAGAGGCAGCCCCAGATACACCATCTACCTTGGATTCG |
| | GCCAGGACCTGAGCGCCGGCAGACCCAAAGAGAAGTCCCTGGTACTGGTGAAGCTAGAGCCCTGGCTGTGTA |
| | GGGTGCATCTGGAAGGCACCCAAAGAGAGGGCGTAAGCTCGCTTGACAGCAGCAGCCTCAGCCTGTGCCTGA |
| | GCAGCGCTAACAGCTTATACGACGACATCGAGTGCTTCCTGATGGAGCTGGAACAACCCGCC |
| | (truncated Hu IRF7 1-151 + 247-503; P038 without epitope tag; null mutation) |
| 219 | ATGGGCGGCCCTCCCGGGCCTTTCCTGGCCCATACACACGCCGGCCTACAGGCTCCTGGCCCTCTGCCCGCCCC |
| | GGCCGGCGACAAGGGCGACCTCCTGCTGCAGGCCGTGCAGCAGTCCTGTCTGGCCGACCACCTGCTGACTGCT |
| | AGCTGGGGCGCCGATCCCGTGCCCACCAAGGCCCCAGGAGAGGGGCAAGAGGGCCTGCCTCTAACCGGCGCA |
| | TGCGCAGGTGGACCAGGCCTCCCCGCCGGCGAGCTGTATGGTTGGGCCGTGGAGACAACCCCCAGCCCCGGC |
| | CCGCAGCCTGCTGCGCTGACCACAGGCGAGGCCGCTGCCCCTGAGAGCCCCCACCAAGCTGAACCCTACCTGA |
| | GCCCCAGCCCCTCTGCCTGCACAGCGGTGCAGGAGCCCAGTCCCGGCGCCTTGGACGTGACCATCATGTATAA |
| | GGGCAGGACTGTGTTACAAAAGGTAGTGGGCCACCCAAGTTGTACCTTTCTGTACGGGCCCCCCGACCCAGCC |
| | GTGCGCGCCACCGACCCCCAGCAGGTGGCCTTCCCCAGCCCCGCTGAGTTGCCCGATCAGAAACAACTCCGGT |
| | ACACCGAGGAATTACTTAGACATGTGGCTCCCGGCCTGCATCTGGAGCTTAGAGGTCCACAGTTGTGGGCCAG |
| | AAGAATGGGCAAGTGCAAGGTTTATTGGGAGGTCGGAGGCCCCCCGGGCAGCGCCAGCCCCAGCACCCCCGC |
| | CTGTCTTCTGCCCAGAAACTGCGACACCCCAATCTTCGATTTCAGAGTGTTTTTCCAGGAACTGGTGGAGTTCAG |
| | AGCAAGGCAAAGAAGAGGCAGCCCTAGATACACCATCTACCTGGGCTTTGGCCAAGACCTGAGCGCCGGCAG |
| | ACCCAAGGAAAAATCCCTGGTCCTGGTGAAACTGGAGCCCTGGCTGTGCAGAGTCCACCTGGAGGGCACCCAG |
| | AGAGAGGGCGTGAGCAGCCTGGACTCGAGCAGCCTGTCCCTGTGTCTGAGCAGCGCGAATTCGCTATATGACG |
| | ACATCGAATGCTTTCTGATGGAGCTGGAACAGCCCGCC |
| | (truncated Hu IRF7 152-503; P039 without epitope tag; null mutation) |
| 220 | ATGCCTCACAGCAGCCTCCACCCTAGCATCCCTTGCCCTAGAGGCCACGGCGCCCAGAAGGCCGCCCTCGTGCT |
| | TTTAAGCGCCTGCTTGGTGACCCTTTGGGGCTTGGGCGAGCCTCCAGAGCACACCTTGAGATATTTGGTGCTCC |
| | ACCTGGCCAGCCTTCAGCTGGGCTTGTTACTCAACGGCGTGTGCAGCCTGGCCGAGGAGCTGAGACACATCCA |
| | CAGCAGATACAGAGGCAGCTACTGGAGAACCGTGAGAGCGTGTCTGGGCTGCCCTCTGAGAAGAGGCGCCTT |
| | GCTTCTTCTCAGTATCTACTTCTACTACTCCCTGCCTAACGCCGTGGGCCCTCCTTTCACCTGGATGCTGGCACTG |
| | CTCGGCCTCAGCCAGGCCCTGAACATCTTGTTGGGCTTGAAGGGCCTGGCCCCTGCCGAGATCAGCGCCGTGT |
| | GCGAGAAGGGCAACTTCAACATGGCCCACGGATTGGCTTGGAGCTACTACATCGGCTACCTGAGACTGATCCT |
| | GCCTGAGCTGCAGGCCAGAATCAGAACCTACAACCAGCACTACAACAACCTGCTGCGCGGCGCAGTGAGCCAG |
| | AGACTGTATATTCTGCTGCCTCTGGACTGCGGCGTGCCTGACAACCTGAGCATGGCCGACCCTAACATCAGATT |
| | CCTGGACAAGCTGCCTCAGCAGACCGGCGACCACGCCGGCATCAAGGACAGAGTGTACAGCAACAGCATCTAT |
| | GAGCTGCTCGAGAATGGCCAGAGAGCCGGCACCTGCGTGCTGGAGTACGCCACCCCTCTGCAGACCCTGTTCG |
| | CCATGAGCCAGTATAGTCAAGCTGGCTTCAGCAGAGAGGACAGACTGGAGCAGGCCAAGCTGTTCTGCAGAA |
| | CCCTGGAGGACATTCTGGCTGACGCCCCTGAGAGCCAGAACAACTGCCGACTGATCGCCTACCAGGAACCAGC |
| | CGACGACAGCAGCTTCAGTCTTTCTCAGGAGGTTCTTCGCCACTTGCGCCAGGAGGAGAAGGAGGAGGTGACC |
| | GTGGGCAGCCTGAAGACCTCCGCAGTCCCTAGCACCAGCACCATGAGTCAGGAGCCGGAGCTATTAATCAGCG |
| | GCATGGAGAAGCCTCTTCCACTCCGAACCGACTTCAGCGCCACCAACTTCAGCCTGCTGAAGCAGGCAGGTGA |
| | CGTTGAGGAGAATCCGGGACCTATGACCGAGTACAAGCTGGTGGTTGTGGGCGCCGACGGCGTGGGCAAGA |
| | GCGCCCTGACCATCCAGCTGATCCAG |
| | (KRAS(G12D)25 mer_nt.STING(V155M)) |
| 221 | ATGACCGAGTACAAGCTAGTAGTCGTGGGCGCCGACGGCGTGGGCAAGAGCGCCCTCACCATCCAGCTAATCC |
| | AGGCCACCAACTTCAGCTTGCTCAAGCAGGCCGGCGACGTGGAGGAGAACCCAGGCCCTATGCCTCACAGCAG |
| | CCTTCACCCTAGCATCCCTTGCCCTAGAGGCCACGGCGCCCAGAAGGCCGCCCTGGTGCTGCTGAGCGCCTGCC |
| | TGGTGACCCTGTGGGGCCTGGGCGAGCCTCCTGAGCACACCCTGAGATATCTGGTGCTTCACCTGGCCAGTTTA |
| | CAGCTGGGCCTGCTTCTTAACGGCGTGTGCAGCCTGGCCGAGGAGCTGAGACACATCCACAGCAGATACAGAG |
| | GCAGCTACTGGAGAACCGTGAGAGCCTGCCTAGGCTGCCCTCTGAGAAGAGGCGCTCTGTTGCTACTTTCCATC |
| | TACTTCTACTACTCCCTGCCTAACGCCGTGGGCCCTCCTTTCACTTGGATGCTGGCGTTGCTGGGTCTGAGCCAG |
| | GCCCTGAACATCCTTCTCGGTCTGAAGGGCCTGGCCCCTGCCGAGATCAGCGCCGTGTGCGAGAAGGGCAACT |
| | TCAACATGGCCCACGGACTCGCCTGGAGCTACTACATCGGCTACCTGAGACTGATCCTGCCTGAGCTGCAGGCC |
| | AGAATCAGAACCTACAACCAGCACTACAACAACCTGCTGCGGGGCGCCGTGAGCCAGAGACTGTATATACTTC |
| | TTCCTCTGGACTGCGGCGTGCCTGACAACCTGAGCATGGCCGACCCTAACATCAGATTCCTGGACAAGCTGCCT |
| | CAGCAGACCGGCGACCACGCCGGCATCAAGGACAGAGTGTACAGCAACTCCATTTATGAGCTGCTCGAGAATG |
| | GCCAGAGAGCCGGCACCTGCGTGCTGGAGTACGCCACCCCTCTGCAGACCCTGTTCGCCATGAGCCAGTACAG |
| | TCAGGCTGGATTCAGCAGAGAGGACAGACTGGAGCAGGCCAAGCTGTTCTGCAGGACACTGGAGGACATACT |
| | AGCAGACGCCCCTGAGAGCCAGAACAACTGCAGACTGATTGCCTACCAGGAGCCTGCGGACGACAGCTCCTTC |
| | AGTCTGAGTCAGGAGGTGTTGCGGCACTTACGCCAAGAAGAGAAGGAGGAGGTGACCGTGGGCAGCCTGAA |
| | GACTAGCGCTGTGCCTAGCACCAGCACAATGTCACAGGAGCCGGAATTGCTAATCAGCGGCATGGAGAAGCCT |
| | CTCCCATTACGTACCGACTTCAGC |
| | (KRAS(G12D)25 mer_ct.STING(V155M)) |
| 222 | ATGCCTCACAGCAGCCTTCACCCTAGCATCCCTTGCCCTAGAGGCCACGGCGCCCAGAAGGCCGCCCTAGTGCT |
| | CCTTAGCGCCTGCCTCGTGACCCTATGGGGCTTAGGCGAGCCTCCAGAGCACACCTTGAGATACCTCGTCCTCC |
| | ACCTGGCTAGTCTACAGCTGGGCCTTCTCCTCAACGGCGTGTGCAGCCTGGCCGAGGAGCTGAGACACATCCA |
| | CAGCAGATACAGAGGCAGCTACTGGAGAACCGTGAGAGCGTGCCTGGGCTGCCCTCTGAGAAGAGGCGCACT |
| | GCTGTTACTCAGCATCTACTTCTACTACTCACTGCCAAACGCCGTGGGCCCTCCTTTCACCTGGATGCTGGCCTT |
| | GCTCGGATTGAGCCAGGCCCTGAACATTTTACTGGGATTGAAGGGCCTGGCCCCTGCCGAGATCAGCGCCGTG |
| | TGCGAGAAGGGCAACTTCAACATGGCCCACGGCCTAGCTTGGAGCTACTACATCGGCTACCTGAGACTGATCCT |
| | GCCTGAGCTGCAGGCCAGAATCAGAACCTACAACCAGCACTACAACAACCTGCTGCGTGGAGCGGTGAGCCAG |
| | AGACTGTATATCCTCCTGCCTCTGGACTGCGGAGTGCCTGACAACCTGAGCATGGCCGACCCTAACATCAGATT |
| | CCTGGACAAGCTGCCTCAGCAGACCGGCGACCACGCCGGCATCAAGGACAGAGTGTACAGCAACTCAATCTAC |
| | GAGCTGTTGGAGAATGGCCAGAGAGCCGGCACCTGCGTGCTGGAGTACGCCACCCCTCTGCAGACCCTGTTCG |
| | CCATGAGCCAGTACTCTCAGGCAGGCTTCAGCAGAGAGGACAGACTGGAGCAGGCCAAGCTGTTCTGCAGAA |
| | CCCTGGAGGACATCCTGGCGGACGCCCCTGAGAGCCAGAACAACTGCCGGCTTATCGCCTACCAGGAGCCAGC |
| | AGACGACAGCAGCTTCTCTCTCTCACAAGAGGTACTGCGCCATCTTCGCCAGGAGGAGAAGGAGGAGGTGACC |
| | GTGGGCAGCCTGAAGACATCCGCCGTACCTAGCACCAGCACCATGTCTCAGGAACCGGAACTGTTGATCAGCG |
| | GCATGGAGAAGCCTCTGCCACTGCGCACCGACTTCAGCGCCACCAACTTCTCCCTACTGAAGCAAGCCGGTGAC |
| | GTTGAAGAGAACCCTGGCCCTATGACCGAGTACAAGCTGGTAGTAGTAGGCGCCGACGGCGTGGGCAAGAGC |
| | GCCCTGACCATCCAGCTGATCCAGATGACTGAATATAAGCTTGTCGTCGTGGGCGCAGATGGCGTTGGTAAGA |
| | GCGCACTTACAATTCAACTCATTCAGATGACGGAGTATAAGCTGGTGGTGGTCGGAGCTGACGGCGTAGGCAA |
| | GAGTGCCCTTACTATTCAGCTAATTCAG |
| | (KRAS(G12D)25 mer^{∧}3_nt.STING(V155M)) |
| 223 | ATGACCGAGTACAAGCTTGTGGTGGTTGGCGCCGACGGCGTGGGCAAGAGCGCCTTAACCATCCAGCTTATCC |
| | AGATGACAGAGTATAAGCTAGTGGTGGTCGGCGCAGACGGAGTGGGAAAGAGTGCATTAACTATTCAACTCA |
| | TCCAAATGACCGAATACAAGCTAGTAGTTGTGGGTGCAGATGGCGTCGGCAAGTCTGCACTGACAATTCAGCT |
| | CATCCAGGCCACCAACTTCAGCCTGCTGAAGCAGGCCGGCGACGTGGAGGAGAACCCTGGCCCTATGCCTCAC |
| | AGCAGCCTGCACCCTAGCATCCCTTGCCCTAGAGGCCACGGCGCCCAGAAGGCCGCCCTGGTGCTGCTGAGCG |
| | CCTGCCTGGTGACCCTGTGGGGCCTGGGCGAGCCTCCTGAGCACACCCTGAGATACCTAGTTTTGCACCTGGCT |
| | TCTCTGCAGCTGGGCCTACTGCTCAACGGCGTGTGCAGCCTGGCCGAGGAGCTGAGACACATCCACAGCAGAT |
| | ACAGAGGCAGCTACTGGAGAACCGTGAGAGCATGCTTAGGCTGCCCTCTGAGAAGAGGCGCTCTGCTCCTCTT |
| | GTCCATCTACTTCTACTACTCGCTACCTAACGCCGTGGGCCCTCCTTTCACCTGGATGCTGGCCCTCTTGGGATTA |
| | AGCCAGGCCCTGAACATCTTGCTGGGACTGAAGGGCCTGGCCCCTGCCGAGATCAGCGCCGTGTGCGAGAAG |
| | GGCAACTTCAACATGGCCCACGGACTCGCTTGGAGCTACTACATCGGCTACCTGAGACTGATCCTGCCTGAGCT |
| | GCAGGCCAGAATCAGAACCTACAACCAGCACTACAACAACCTGCTGCGGGGAGCAGTGAGCCAGAGACTGTA |
| | TATTCTGCTCCCTCTGGACTGCGGCGTGCCTGACAACCTGAGCATGGCCGACCCTAACATCAGATTCCTGGACA |
| | AGCTGCCTCAGCAGACCGGCGACCACGCCGGCATCAAGGACAGAGTGTACAGCAACAGCATTTACGAGCTGCT |
| | GGAGAACGGCCAGAGAGCCGGCACCTGCGTGCTGGAGTACGCCACCCCTCTGCAGACCCTGTTCGCCATGAGC |
| | CAGTACTCCCAGGCAGGATTCAGCAGAGAGGACAGACTGGAGCAGGCCAAGCTGTTCTGCCGTACTCTTGAGG |
| | ACATCCTTGCAGACGCCCCTGAGAGCCAGAACAACTGCCGGTTGATTGCCTACCAGGAACCGGCAGACGACAG |
| | CTCATTCTCCTTGTCTCAGGAGGTCCTTAGACACCTGCGGCAGGAGGAGAAGGAGGAGGTGACCGTGGGCAG |
| | CCTGAAGACATCCGCCGTGCCTAGCACGTCTACCATGTCCCAGGAGCCGGAACTGCTAATCAGCGGCATGGAG |
| | AAGCCTCTGCCTCTCAGGACCGACTTCAGC |
| | (KRAS(G12D)25 mer^{∧}3_ct.STING(V155M)) |
| 224 | MPHSSLHPSIPCPRGHGAQKAALVLLSACLVTLWGLGEPPEHTLRYLVLHLASLQLGLLLNGVCSLAEELRHIHSRYRG |
| | SYWRTVRACLGCPLRRGALLLLSIYFYYSLPNAVGPPFTWMLALLGLSQALNILLGLKGLAPAEISAVCEKGNFNVAH |
| | GLAWSYYIGYLRLILPELQARIRTYNQHYNNLLRGAVSQRLYILLPLDCGVPDNLSMADPNIRFLDKLPQQTGDHAGI |
| | KDRVYSNSIYELLENGQRAGTCVLEYATPLQTLFAMSQYSQAGFSREDKLEQAKLFCRTLEDILADAPESQNNCRLIAY |
| | QEPADDSSFSLSQEVLRHLRQEEKEEVTVGSLKTSAVPSTSTMSQEPELLISGMEKPLPLRTDFST |
| | (Hu STING (R284K) var; no epitope tag) |
| 225 | ATGCCCCATAGCAGCCTGCACCCCAGCATCCCCTGCCCCAGAGGCCACGGCGCCCAGAAGGCCGCCCTGGTCCT |
| | GCTGAGCGCATGCCTGGTCACCCTGTGGGGCCTGGGCGAGCCCCCCGAGCACACCCTGAGATACCTGGTGCTG |
| | CACCTCGCCAGCCTGCAGCTGGGCCTGCTGCTGAACGGCGTGTGCAGCCTGGCCGAGGAGCTGAGACACATCC |
| | ACAGCAGATATAGAGGCAGCTACTGGAGAACCGTGAGAGCTTGCCTCGGCTGCCCCCTGAGAAGAGGCGCCC |
| | TGCTGCTGCTGAGCATCTACTTTTACTACAGCCTGCCCAACGCTGTGGGCCCCCCTTTCACGTGGATGCTCGCCC |
| | TGCTGGGACTGAGCCAGGCCCTGAACATCCTGCTGGGCCTTAAGGGCCTAGCCCCCGCCGAGATCAGCGCCGT |
| | GTGCGAGAAGGGCAACTTCAATGTGGCCCACGGCCTGGCCTGGAGCTACTACATCGGCTACCTGAGACTGATC |
| | CTGCCCGAGCTGCAGGCCAGAATCAGAACCTACAATCAGCACTACAACAACCTGCTGAGAGGCGCCGTGAGCC |
| | AGAGACTGTACATCCTGCTGCCCCTGGACTGCGGCGTGCCCGACAACCTCAGCATGGCCGACCCCAACATCAG |
| | ATTCCTGGACAAGCTGCCCCAGCAGACCGGCGACCACGCCGGCATCAAGGATCGCGTGTACAGCAACAGCATC |
| | TACGAGCTGCTGGAAAACGGCCAGAGAGCCGGAACCTGCGTGCTGGAGTACGCCACACCCCTGCAGACCCTGT |
| | TCGCCATGAGCCAGTACAGCCAGGCCGGCTTCAGCAGAGAGGACAAGCTGGAGCAGGCCAAGCTGTTCTGCA |
| | GAACCCTGGAGGATATCCTCGCCGACGCCCCCGAGAGCCAGAACAACTGCAGGCTGATCGCGTACCAGGAGCC |
| | CGCTGACGACAGCAGCTTTAGCCTGAGCCAGGAGGTGCTGAGACATCTGCGTCAAGAGGAAAAGGAGGAGGT |
| | GACCGTGGGCTCCCTGAAGACCAGCGCCGTGCCCAGCACCAGCACCATGAGCCAGGAGCCCGAGCTGCTGATC |
| | AGCGGCATGGAGAAGCCACTGCCCCTCAGAACCGACTTCAGCACC |
| | (Hu STING (R284K) var; no epitope tag) |
| 226 | EGAMVAATQGAAAAAGSGAGTGGGTASGGTEGGSAESEGAK |
| | Cathepsin B sensitive site |
| 227 | AMVAATQGAAAAAGSGAGTGGGTASGGTEGGSAESEGAK |
| | Cathepsin B sensitive site |
| 228 | GGGGGGGGAGAAGGGGGGENYDDPHK |
| | Cathepsin B sensitive site |
| 229 | MVAATQGAAAAAGSGAGTGGGTASGGTEGGSAESEGAK |
| | Cathepsin B sensitive site |
| 230 | QLLCGAAIGTHEDDKYR |
| | Cathepsin B sensitive site |
| 231 | FSHHFEDADNIYIFLELCSRKS |
| | Cathepsin B sensitive site |
| 232 | YXLVGAGAIGCELLK |
| | Cathepsin B sensitive site |
| 233 | IPESCSFGYHAGGWGKPPVDETGKPL |
| | Cathepsin B sensitive site |
| 234 | VAATQGAAAAAGSGAGTGGGTASGGTEGGSASEGAK |
| | Cathepsin B sensitive site |
| 235 | SEADIEGPLPAKDIHLDLPSNN |
| | Cathepsin B sensitive site |
| 236 | HFNALGGWGELQNSVK |
| | Cathepsin B sensitive site |
| 237 | FAQALGLTEAVK |
| | Cathepsin B sensitive site |
| 238 | TSVLAAANPIESQWNPK |
| | Cathepsin B sensitive site |
| 239 | QLLQANPILESFGNAK |
| | Cathepsin B sensitive site |
| 240 | TSILAAANPISGHYDR |
| | Cathepsin B sensitive site |
| 241 | IXXANPLLEAFGNAK |
| | Cathepsin B sensitive site |
| 242 | LYGAQFHPEVGLTENGK |
| | Cathepsin B sensitive site |
| 243 | PQGQAPPLSQAQGHPGIQTPQR |
| | Cathepsin B sensitive site |
| 244 | AAASAAAASAASGSPGPGEGSAGGEKR |
| | Cathepsin B sensitive site |
| 245 | IXXXFLGASLKDEVLK |
| | Cathepsin B sensitive site |
| 246 | LTISPDYAYGATGHPGIIPPH |
| | Cathepsin B sensitive site |
| 247 | LTISPDYAYGATGHPGIIPPHA |
| | Cathepsin B sensitive site |
| 248 | ILISLATGHREEGGENLDQ |
| | Cathepsin B sensitive site |
| 249 | LSELTQQLAQATGKPPQYIAVHVVPDQ |
| | Cathepsin B sensitive site |
| 250 | LSELTQQLAQATGKPPQYIAVHVVPDQL |
| | Cathepsin B sensitive site |
| 251 | DATNVGDEGGFAPNILENK |
| | Cathepsin B sensitive site |
| 252 | ILAQATSDLVNAIK |
| | Cathepsin B sensitive site |
| 253 | VXXVXQHAVGIVVNK |
| | Cathepsin B sensitive site |
| 254 | GSLAEAVGSPPPAATPTPTPPTR |
| | Cathepsin B sensitive site |
| 255 | SXGLPVGAVINCADNTGAK |
| | Cathepsin B sensitive site |
| 256 | YCFSEMAPVCAVVGGILAQEIVK |
| | Cathepsin B sensitive site |
| 257 | HVYGYSMAYGPAQHAISTEK |
| | Cathepsin B sensitive site |
| 258 | LWQLSKPRPGCSVLGPLPLL |
| | Cathepsin B sensitive site |
| 259 | MILIQDGSQNTNVDKPLR |
| | Cathepsin B sensitive site |
| 260 | TYSMVVVPLYDTLGPGAIRYII |
| | Cathepsin B sensitive site |
| 261 | HFAMMHGGTGFAGIDSSSPEVK |
| | Cathepsin B sensitive site |
| 262 | GXLKPGMVVTFAPVNVTTEVK |
| | Cathepsin B sensitive site |
| 263 | FNALFAQGNYSEAAK |
| | Cathepsin B sensitive site |
| 264 | GPIHIGGPPGFASSSGKPGPTVIK |
| | Cathepsin B sensitive site |
| 265 | GFGFVTFDDHDPVDK |
| | Cathepsin B sensitive site |
| 266 | DQGSCGSCWAFGAVEAISDR |
| | Cathepsin B sensitive site |
| 267 | GXNFGFGDSRGGGGNFGPGPG |
| | Cathepsin B sensitive site |
| 268 | HDLFDSGFGGGAGVETGGK |
| | Cathepsin B sensitive site |
| 269 | CYLFGGLANDSEDPK |
| | Cathepsin B sensitive site |
| 270 | TTEDSVMLNGFGTVVNALGK |
| | Cathepsin B sensitive site |
| 271 | LTEGLHGFHVHEFGDNTAGC |
| | Cathepsin B sensitive site |
| 272 | GYAFIEYEHER |
| | Cathepsin B sensitive site |
| 273 | MFIGGLSWDTSKK |
| | Cathepsin B sensitive site |
| 274 | MFIGGLSWDTTKK |
| | Cathepsin B sensitive site |
| 275 | SMGFIGHYLDQK |
| | Cathepsin B sensitive site |
| 276 | SMGFIGHYLDQK |
| | Cathepsin B sensitive site |
| 277 | ALXGGIGFIHHNCTPEFQANE |
| | Cathepsin B sensitive site |
| 278 | NLQSTFSGFGFINSENVFK |
| | Cathepsin B sensitive site |
| 279 | GFCFITYTDEEPVKK |
| | Cathepsin B sensitive site |
| 280 | MPMFIVNTNVPR |
| | Cathepsin B sensitive site |
| 281 | VSEIFVELQGFLAAEQDIR |
| | Cathepsin B sensitive site |
| 282 | GFCFLEYEDHK |
| | Cathepsin B sensitive site |
| 283 | QAVSMFLGAVEEAKK |
| | Cathepsin B sensitive site |
| 284 | KPXKPMQFLGDEETVRK |
| | Cathepsin B sensitive site |
| 285 | GAAEPHTIAAFLGGAAAQEVIK |
| | Cathepsin B sensitive site |
| 286 | MIPCDFLIPVQTQHPIR |
| | Cathepsin B sensitive site |
| 287 | QGAPTSFLPPEASQLKPDR |
| | Cathepsin B sensitive site |
| 288 | STGGAPTFNVTVTK |
| | Cathepsin B sensitive site |
| 289 | MVYMFQYDSTHGK |
| | Cathepsin B sensitive site |
| 290 | HFPMTHGNTGFSGIESSSPEVK |
| | Cathepsin B sensitive site |
| 291 | AVAFSPVTELKK |
| | Cathepsin B sensitive site |
| 292 | GFGFVTFSSMAEVDAAMAARPH |
| | Cathepsin B sensitive site |
| 293 | TCGFDFTGAVEDISK |
| | Cathepsin B sensitive site |
| 294 | EYSGLSDGYGFTTDLFGR |
| | Cathepsin B sensitive site |
| 295 | GQHVXGSPFQFTVGPLGEGGAHK |
| | Cathepsin B sensitive site |
| 296 | GFGFVDFNSEEDAK |
| | Cathepsin B sensitive site |
| 297 | FXFVEFEDPR |
| | Cathepsin B sensitive site |
| 298 | FXFVEFEDPR |
| | Cathepsin B sensitive site |
| 299 | IELFVGGELIDPADDRK |
| | Cathepsin B sensitive site |
| 300 | MFVGGLSWDTSKK |
| | Cathepsin B sensitive site |
| 301 | AFSAFVGQMHQQGILK |
| | Cathepsin B sensitive site |
| 302 | GILFVGSGVSGGEEGAR |
| | Cathepsin B sensitive site |
| 303 | IIAFVGSPVEDNEKDLVK |
| | Cathepsin B sensitive site |
| 304 | DYAFVHFEDR |
| | Cathepsin B sensitive site |
| 305 | GYAFVHFETQEAADK |
| | Cathepsin B sensitive site |
| 306 | GYGFVHFETQEAAER |
| | Cathepsin B sensitive site |
| 307 | NYGFVHIEDK |
| | Cathepsin B sensitive site |
| 308 | ITLPVDFVTADKFDENAK |
| | Cathepsin B sensitive site |
| 309 | GFGFVTFDDHDPVDK |
| | Cathepsin B sensitive site |
| 310 | LPNFGFVVFDDSEPVQK |
| | Cathepsin B sensitive site |
| 311 | GFGFVYFQNHDAADK |
| | Cathepsin B sensitive site |
| 312 | YQFWDTQPVPK |
| | Cathepsin B sensitive site |
| 313 | QLLCGAAIGTHEDDKYR |
| | Cathepsin B sensitive site |
| 314 | QLLCGAAIGTHEDDKYR |
| | Cathepsin B sensitive site |
| 315 | PPAGGGGGAGGAGGGPPPGPPGAGDR |
| | Cathepsin B sensitive site |
| 316 | FGGSFAGSFGGAGGHAPGVAR |
| | Cathepsin B sensitive site |
| 317 | CNPIISGLYQGAGGPGPGGFGAQGPK |
| | Cathepsin B sensitive site |
| 318 | PGLNLPPPIGGAGPPLGLPKPK |
| | Cathepsin B sensitive site |
| 319 | QPXVDGFLVGGASLKPEFVDIINAK |
| | Cathepsin B sensitive site |
| 320 | VTGDHIPTPQDLPQR |
| | Cathepsin B sensitive site |
| 321 | YGGELVPHFPAR |
| | Cathepsin B sensitive site |
| 322 | YQGAGGPGPGGFGAQGPK |
| | Cathepsin B sensitive site |
| 323 | EYFGGFGEVESIELPMDNK |
| | Cathepsin B sensitive site |
| 324 | ALVLGGFAHMDTETK |
| | Cathepsin B sensitive site |
| 325 | VSHVSTGGGASLELLEGK |
| | Cathepsin B sensitive site |
| 326 | AEGGGGGGRPGAPAAGDGK |
| | Cathepsin B sensitive site |
| 327 | RGGGGGGSGGIGYPYPR |
| | Cathepsin B sensitive site |
| 328 | NMGGPYGGGNYGPGGSGGSGGYG |
| | Cathepsin B sensitive site |
| 329 | GTGGVDTAATGGVFDISNLDR |
| | Cathepsin B sensitive site |
| 330 | HFNALGGWGELQNSVK |
| | Cathepsin B sensitive site |
| 331 | PESCSFGYHAGGWGKPPVDETGKPL |
| | Cathepsin B sensitive site |
| 332 | SSLPNFCGIFNHLER |
| | Cathepsin B sensitive site |
| 333 | AMALXGGIGFIHHNCTPEF |
| | Cathepsin B sensitive site |
| 334 | AMALXGGIGFIHHNCTPEFQANE |
| | Cathepsin B sensitive site |
| 335 | EWIKPIMFSGGIGSMEADHISK |
| | Cathepsin B sensitive site |
| 336 | GDGPVQGIINFEQK |
| | Cathepsin B sensitive site |
| 337 | EMAPVCAVVGGILAQEIVK |
| | Cathepsin B sensitive site |
| 338 | LAFHGILLHGLEDR |
| | Cathepsin B sensitive site |
| 339 | MGVVAGILVQNVLK |
| | Cathepsin B sensitive site |
| 340 | FTASAGIQVVGDDLTVTNPK |
| | Cathepsin B sensitive site |
| 341 | TPYQIACGISQGLADNTVIAK |
| | Cathepsin B sensitive site |
| 342 | YPIEHGIVTNWDDMEK |
| | Cathepsin B sensitive site |
| 343 | VASGIPAGWXGLDCGPESSKK |
| | Cathepsin B sensitive site |
| 344 | LFVGGLDWSTTQETLR |
| | Cathepsin B sensitive site |
| 345 | HGGSLGLGLAAMGTAR |
| | Cathepsin B sensitive site |
| 346 | IFVGGLSANTVVEDVK |
| | Cathepsin B sensitive site |
| 347 | LFIGGLSFETTDDSLR |
| | Cathepsin B sensitive site |
| 348 | LFIGGLSFETTDESLR |
| | Cathepsin B sensitive site |
| 349 | LFIGGLSFETTEESLR |
| | Cathepsin B sensitive site |
| 350 | MFXGGLSWDTSKK |
| | Cathepsin B sensitive site |
| 351 | DAVSGMGVIVHIIEK |
| | Cathepsin B sensitive site |
| 352 | GGNFGFGDSR |
| | Cathepsin B sensitive site |
| 353 | GTTGSGAGSGGPGGLTSAAPAGGDKK |
| | Cathepsin B sensitive site |
| 354 | IISGLYQGAGGPGPGGFGAQGPK |
| | Cathepsin B sensitive site |
| 355 | IISGLYQGAGGPGPGGFGAQGPK |
| | Cathepsin B sensitive site |
| 356 | GGGLLIGGQAWDWANQGEDERV |
| | Cathepsin B sensitive site |
| 357 | GNFGGSFAGSFGGAGGHAPGVAR |
| | Cathepsin B sensitive site |
| 358 | NFGGSFAGSFGGAGGHAPGVAR |
| | Cathepsin B sensitive site |
| 359 | SAADTKPGTTGSGAGSGGPGGLTSAAPAGGDKK |
| | Cathepsin B sensitive site |
| 360 | AATQGAAAAAGSGAGTGGGTASGGTEGGSAESEGAK |
| | Cathepsin B sensitive site |
| 361 | GSSGGSGAKPSDAASEAAR |
| | Cathepsin B sensitive site |
| 362 | IQFHFHWGSLDGQGSEHTVDK |
| | Cathepsin B sensitive site |
| 363 | MILIQDGSQNTNVDKPLR |
| | Cathepsin B sensitive site |
| 364 | KGTFTDDLHK |
| | Cathepsin B sensitive site |
| 365 | QQSHFAMMHGGTGFAGIDSSSPEVK |
| | Cathepsin B sensitive site |
| 366 | VAVLISGTGSNLQALIDSTR |
| | Cathepsin B sensitive site |
| 367 | FLAAGTHLGGTNLDFQ |
| | Cathepsin B sensitive site |
| 368 | LVLGTHTSDEQNHL |
| | Cathepsin B sensitive site |
| 369 | TGGVDTAATGGVFDISNLDR |
| | Cathepsin B sensitive site |
| 370 | TGGVDTAAVGGVFDVSNADR |
| | Cathepsin B sensitive site |
| 371 | AVXIVAAGVGEFEAGISK |
| | Cathepsin B sensitive site |
| 372 | EILTLLQGVHQGAGFQDIPK |
| | Cathepsin B sensitive site |
| 373 | MKPLMGVIYVPLTDKEK |
| | Cathepsin B sensitive site |
| 374 | ECISXHVGQAGVQIGNACWE |
| | Cathepsin B sensitive site |
| 375 | HFNALGGWGELQNSVK |
| | Cathepsin B sensitive site |
| 376 | ESCSFGYHAGGWGKPPVDETGKPL |
| | Cathepsin B sensitive site |
| 377 | AGYVTHLMK |
| | Cathepsin B sensitive site |
| 378 | TMFSSEVQFGHAGACANQASETAVAK |
| | Cathepsin B sensitive site |
| 379 | MPFPVNHGASSEDTLLK |
| | Cathepsin B sensitive site |
| 380 | FFLHHLIAEIHTAEIRAT |
| | Cathepsin B sensitive site |
| 381 | NXSAXQVLIEHIGNLDR |
| | Cathepsin B sensitive site |
| 382 | GGYVLHIGTIYGDLK |
| | Cathepsin B sensitive site |
| 383 | DXHLGGEDFDNR |
| | Cathepsin B sensitive site |
| 384 | GILGPPPPSFHLGGPAVGPR |
| | Cathepsin B sensitive site |
| 385 | PTPPPTLHLVPEPAAPPPP |
| | Cathepsin B sensitive site |
| 386 | YGPQYGHPPPPPPPPEYGPHADSPV |
| | Cathepsin B sensitive site |
| 387 | KHSGPNSADSANDGFVR |
| | Cathepsin B sensitive site |
| 388 | RPELLTHSTTEVTQPR |
| | Cathepsin B sensitive site |
| 389 | LXGHVGFDSLPDQLVNK |
| | Cathepsin B sensitive site |
| 390 | AASATQTIAAAQHAASTPK |
| | Cathepsin B sensitive site |
| 391 | CLTQSGIAGGYKPF |
| | Cathepsin B sensitive site |
| 392 | ELAQIAGRPTEDEDEKEK |
| | Cathepsin B sensitive site |
| 393 | AITIAGVPQSVTECVK |
| | Cathepsin B sensitive site |
| 394 | GLCAIAQAESLR |
| | Cathepsin B sensitive site |
| 395 | KPTALIGVAAIGGAFSEQILK |
| | Cathepsin B sensitive site |
| 396 | DYMNVQCHACIGGTNVGEDIR |
| | Cathepsin B sensitive site |
| 397 | NTQNFQSLHNIGSVVQHSEGKPL |
| | Cathepsin B sensitive site |
| 398 | LKPPTLIHGQAPSAGLPSQKPK |
| | Cathepsin B sensitive site |
| 399 | VLIIGGGDGGVLR |
| | Cathepsin B sensitive site |
| 400 | GCITIIGGGDTATCCAK |
| | Cathepsin B sensitive site |
| 401 | GRPSETGIIGIIDPECR |
| | Cathepsin B sensitive site |
| 402 | EAFGWHAIIVDGHSVEELCK |
| | Cathepsin B sensitive site |
| 403 | LAAAILGGVDQIHIKPG |
| | Cathepsin B sensitive site |
| 404 | LYSILGTTLKDEGK |
| | Cathepsin B sensitive site |
| 405 | MILIQDGSQNTNVDKPLR |
| | Cathepsin B sensitive site |
| 406 | LAMQEFMILPVGAANFR |
| | Cathepsin B sensitive site |
| 407 | VPYLIAGIQHSCQDIGAK |
| | Cathepsin B sensitive site |
| 408 | TVAGGVHISGLHTESAPR |
| | Cathepsin B sensitive site |
| 409 | VAVLISGTGSNLQALIDSTR |
| | Cathepsin B sensitive site |
| 410 | GITAIGGTSTISSEGTQHSYSEEEK |
| | Cathepsin B sensitive site |
| 411 | AGVSISVVHGNLSEEAAK |
| | Cathepsin B sensitive site |
| 412 | HVTQAHVQTGITAAPPPHPGAPHPPQ |
| | Cathepsin B sensitive site |
| 413 | AGLFLPGSVGITDPCESGNFR |
| | Cathepsin B sensitive site |
| 414 | AFAHITGGGLLENIPR |
| | Cathepsin B sensitive site |
| 415 | ILAQITGTEHLK |
| | Cathepsin B sensitive site |
| 416 | TFXNITPAEVGVLVGK |
| | Cathepsin B sensitive site |
| 417 | HSSGIVADLSEQSLK |
| | Cathepsin B sensitive site |
| 418 | EDGNEEDKENQGDETQGQQPPQR |
| | Cathepsin B sensitive site |
| 419 | PGPSGITIPGKPGAQGVPGPPG |
| | Cathepsin B sensitive site |
| 420 | GLTKPAALAAAPAKPGGAGGSK |
| | Cathepsin B sensitive site |
| 421 | LGAQLADLHLDNK |
| | Cathepsin B sensitive site |
| 422 | SLVASLAEPDFVVTDFAK |
| | Cathepsin B sensitive site |
| 423 | MSLPLLAGGVADDINTNKK |
| | Cathepsin B sensitive site |
| 424 | QPYAVSELAGHQTSAESWGTGR |
| | Cathepsin B sensitive site |
| 425 | VTVAGLAGKDPVQC |
| | Cathepsin B sensitive site |
| 426 | IITLAGPTNAIFK |
| | Cathepsin B sensitive site |
| 427 | STHGLAILGPENPK |
| | Cathepsin B sensitive site |
| 428 | ASAELALGENSEVLK |
| | Cathepsin B sensitive site |
| 429 | ILISLATGHREEGGENLDQ |
| | Cathepsin B sensitive site |
| 430 | AMSRPFGVALLFGGVDEK |
| | Cathepsin B sensitive site |
| 431 | LQATAHAQAQLGCPVIIHPGR |
| | Cathepsin B sensitive site |
| 432 | ILAGLGFDPEMQNRPT |
| | Cathepsin B sensitive site |
| 433 | PERPQQLPHGLGGIGMGLGPGGQPIDANHLNK |
| | Cathepsin B sensitive site |
| 434 | QLMQLIGPAGLGGLGGLGALTGPG |
| | Cathepsin B sensitive site |
| 435 | HFNALGGWGELQNSVK |
| | Cathepsin B sensitive site |
| 436 | MGAGLGHGMDR |
| | Cathepsin B sensitive site |
| 437 | THMTAIVGMALGHRPIPNQPPT |
| | Cathepsin B sensitive site |
| 438 | PHGLGGIGMGLGPGGQPIDANHLNK |
| | Cathepsin B sensitive site |
| 439 | ASQGDSISSQLGPIHPPPR |
| | Cathepsin B sensitive site |
| 440 | VWQLGSSSPNFTLEGHEK |
| | Cathepsin B sensitive site |
| 441 | YVATLGVEVHPL |
| | Cathepsin B sensitive site |
| 442 | KLIADYSPDDIFN |
| | Cathepsin B sensitive site |
| 443 | TXGLIFVVDSNDR |
| | Cathepsin B sensitive site |
| 444 | VPEFQFLIGDEAATHLK |
| | Cathepsin B sensitive site |
| 445 | CNINLLPLPDPIPSGLME |
| | Cathepsin B sensitive site |
| 446 | LITEMVALNPDFKPPADYKPPA |
| | Cathepsin B sensitive site |
| 447 | NQVALNPQNTVFDAK |
| | Cathepsin B sensitive site |
| 448 | GLLKPGLNVVLEGPK |
| | Cathepsin B sensitive site |
| 449 | GVNLPGAAVDLPAVSEK |
| | Cathepsin B sensitive site |
| 450 | ISXGLPVGAVINCADNTGAK |
| | Cathepsin B sensitive site |
| 451 | GQVCLPVISAENWK |
| | Cathepsin B sensitive site |
| 452 | EILTLLQGVHQGAGFQDIPK |
| | Cathepsin B sensitive site |
| 453 | NNQFQALLQYADPVSAQHAK |
| | Cathepsin B sensitive site |
| 454 | LFIGGLSFETTDDSLR |
| | Cathepsin B sensitive site |
| 455 | AIQLSGAEQLEALK |
| | Cathepsin B sensitive site |
| 456 | DVSIEDSVISLSGDHCIIGR |
| | Cathepsin B sensitive site |
| 457 | EYLLSGDISEAEHCLK |
| | Cathepsin B sensitive site |
| 458 | VVISSDGQFALSGSWDGTLR |
| | Cathepsin B sensitive site |
| 459 | VHEQLAALSQGPISKPK |
| | Cathepsin B sensitive site |
| 460 | LVXLXXETALLSSGFSLEDPQTH |
| | Cathepsin B sensitive site |
| 461 | GPDGLTAFEATDNQAIK |
| | Cathepsin B sensitive site |
| 462 | ALYWLSGLTCTEQNFISK |
| | Cathepsin B sensitive site |
| 463 | IITLTGPTNAIFK |
| | Cathepsin B sensitive site |
| 464 | LATQLTGPVMPVR |
| | Cathepsin B sensitive site |
| 465 | FPSLLTHNENMVAK |
| | Cathepsin B sensitive site |
| 466 | LEXLXTINXGLTSIANLPK |
| | Cathepsin B sensitive site |
| 467 | ALLLLLVGGVDQSPR |
| | Cathepsin B sensitive site |
| 468 | GKPVGLVGVTELSDAQKK |
| | Cathepsin B sensitive site |
| 469 | VNVAGLVLAGSADFK |
| | Cathepsin B sensitive site |
| 470 | QGYIGAALVLGGVDVTGPH |
| | Cathepsin B sensitive site |
| 471 | LYTLVLTDPDAPSR |
| | Cathepsin B sensitive site |
| 472 | AQIHDLVLVGGSTR |
| | Cathepsin B sensitive site |
| 473 | LNHVAAGLVSPSLKSDTSSK |
| | Cathepsin B sensitive site |
| 474 | IEVGLVVGNSQVAFEK |
| | Cathepsin B sensitive site |
| 475 | GYHQSASEHGLVVIAPDTSPR |
| | Cathepsin B sensitive site |
| 476 | GYHQSASEHGLVVIAPDTSPR |
| | Cathepsin B sensitive site |
| 477 | QDHPWLLSQNLVVKPDQLIK |
| | Cathepsin B sensitive site |
| 478 | MGLAMGGGGGASFDR |
| | Cathepsin B sensitive site |
| 479 | QLPHGLGGIGMGLGPGGQPIDANHLNK |
| | Cathepsin B sensitive site |
| 480 | VVVLMGSTSDLGHCEK |
| | Cathepsin B sensitive site |
| 481 | MALIQMGSVEEAVQA |
| | Cathepsin B sensitive site |
| 482 | TTGFGMIYDSLDYAK |
| | Cathepsin B sensitive site |
| 483 | WLLAEMLGDLSDSQLK |
| | Cathepsin B sensitive site |
| 484 | QAQYLGMSCDGPFKPDH |
| | Cathepsin B sensitive site |
| 485 | AHSSMVGVNLPQK |
| | Cathepsin B sensitive site |
| 486 | SGPVVAMVWEGLNVVK |
| | Cathepsin B sensitive site |
| 487 | VNTQNFQSLHNIGSVVQHSEGKPL |
| | Cathepsin B sensitive site |
| 488 | LYVSNLGIGHTR |
| | Cathepsin B sensitive site |
| 489 | VYVGNLGNNGNKTELER |
| | Cathepsin B sensitive site |
| 490 | IVDLLQMLEMNMAIAFPA |
| | Cathepsin B sensitive site |
| 491 | VLAQNSGFDLQETLVK |
| | Cathepsin B sensitive site |
| 492 | QQSHFPMTHGNTGFSGIESSSPEVK |
| | Cathepsin B sensitive site |
| 493 | ILIANTGMDTDKIK |
| | Cathepsin B sensitive site |
| 494 | NNTVTPGGKPNK |
| | Cathepsin B sensitive site |
| 495 | VVNVANVGAVPSGQDNIHR |
| | Cathepsin B sensitive site |
| 496 | MPFPVNHGASSEDTLLK |
| | Cathepsin B sensitive site |
| 497 | RPKDPGHPY |
| | Cathepsin B sensitive site |
| 498 | ELDIMEPKVPDDIYK |
| | Cathepsin B sensitive site |
| 499 | AETSQQEASEGGDPASPALSLS |
| | Cathepsin B sensitive site |
| 500 | LLAAQNPLSQADRPHQ |
| | Cathepsin B sensitive site |
| 501 | PDNFXFGQSGAGNNWAK |
| | Cathepsin B sensitive site |
| 502 | MIAGQVLDINLAAEPK |
| | Cathepsin B sensitive site |
| 503 | IILNSHSPAGSAAISQQDFHPK |
| | Cathepsin B sensitive site |
| 504 | GAVAVSAAPGSAAPAAGSAPAAAEEK |
| | Cathepsin B sensitive site |
| 505 | SAAGAAGSAGGSSGAAGAAGGGAGAGTRPGDGGTASAGAAGPGAATK |
| | Cathepsin B sensitive site |
| 506 | FTASAGIQVVGDDLTVTNPK |
| | Cathepsin B sensitive site |
| 507 | FGIVTSSAGTGTTEDTEAKK |
| | Cathepsin B sensitive site |
| 508 | SLYQSAGVAPESFEYIEAHGTGTK |
| | Cathepsin B sensitive site |
| 509 | VSEIDEMFEARKM |
| | Cathepsin B sensitive site |
| 510 | FGGSFAGSFGGAGGHAPG |
| | Cathepsin B sensitive site |
| 511 | FGGSFAGSFGGAGGHAPGVAR |
| | Cathepsin B sensitive site |
| 512 | AADTKPGTTGSGAGSGGPGGLTSAAPAGGDKK |
| | Cathepsin B sensitive site |
| 513 | ATQGAAAAAGSGAGTGGGTASGGTEGGSAESEGAK |
| | Cathepsin B sensitive site |
| 514 | EIELIGSGGFGQVFK |
| | Cathepsin B sensitive site |
| 515 | KPGTTGSGAGSGGPGGLTSAAPAGGDKK |
| | Cathepsin B sensitive site |
| 516 | LYANXVXSGGTTMYPGIADR |
| | Cathepsin B sensitive site |
| 517 | RSGKYDLDFK |
| | Cathepsin B sensitive site |
| 518 | HDGYGSHGPLLPLPSR |
| | Cathepsin B sensitive site |
| 519 | SLFSSIGEVESAK |
| | Cathepsin B sensitive site |
| 520 | LQSIGTENTEENR |
| | Cathepsin B sensitive site |
| 521 | SLVASLAEPDFVVTDFAK |
| | Cathepsin B sensitive site |
| 522 | AEPMGEKPVGSLAGIGEVLGK |
| | Cathepsin B sensitive site |
| 523 | VQEAINSLGGSVFPK |
| | Cathepsin B sensitive site |
| 524 | SAAAASAASGSPGPGEGSAGGEKR |
| | Cathepsin B sensitive site |
| 525 | QTIDNSQGAYQEAFDISKK |
| | Cathepsin B sensitive site |
| 526 | FGIVTSSAGTGTTEDTEAK |
| | Cathepsin B sensitive site |
| 527 | FGIVTSSAGTGTTEDTEAKK |
| | Cathepsin B sensitive site |
| 528 | XSSFDLDYDFQR |
| | Cathepsin B sensitive site |
| 529 | FQAGTSKPLHSSGINVNAAPF |
| | Cathepsin B sensitive site |
| 530 | HIGGPPGFASSSGKPGPTVIK |
| | Cathepsin B sensitive site |
| 531 | XSSGPGASSGTSGDHGELVVR |
| | Cathepsin B sensitive site |
| 532 | ELVSSSSSGSDSDSEVDKK |
| | Cathepsin B sensitive site |
| 533 | MDSTEPPYSQKR |
| | Cathepsin B sensitive site |
| 534 | VVVLMGSTSDLGHCEK |
| | Cathepsin B sensitive site |
| 535 | ILDSVGIEADDDRINK |
| | Cathepsin B sensitive site |
| 536 | STQPISSVGKPASVIK |
| | Cathepsin B sensitive site |
| 537 | ALQSVGQIVGEVLK |
| | Cathepsin B sensitive site |
| 538 | VSSLAEGSVTSVGSVNPAENFR |
| | Cathepsin B sensitive site |
| 539 | TGSISSSVSVPAKPER |
| | Cathepsin B sensitive site |
| 540 | YXXXXXXYSQSYGGYENQK |
| | Cathepsin B sensitive site |
| 541 | IYWGTATTGKPHV |
| | Cathepsin B sensitive site |
| 542 | MVQTAVVPVKK |
| | Cathepsin B sensitive site |
| 543 | MMLGTEGGEGFVVK |
| | Cathepsin B sensitive site |
| 544 | XTFIAIKPDGVQR |
| | Cathepsin B sensitive site |
| 545 | VSHVSTGGGASLELLEGK |
| | Cathepsin B sensitive site |
| 546 | AAAAAGSGAGTGGGTASGGTEGGSAESEGAK |
| | Cathepsin B sensitive site |
| 547 | TIGNSCGTIGLIHAVANNQDK |
| | Cathepsin B sensitive site |
| 548 | TGEEIFGTIGMRPNAK |
| | Cathepsin B sensitive site |
| 549 | TTQFSCTLGEKFEETTADGR |
| | Cathepsin B sensitive site |
| 550 | GCTATLGNFAK |
| | Cathepsin B sensitive site |
| 551 | YVATLGVEVHPL |
| | Cathepsin B sensitive site |
| 552 | LAATNALLNSLEFTK |
| | Cathepsin B sensitive site |
| 553 | GPGASSGTSGDHGELVVR |
| | Cathepsin B sensitive site |
| 554 | STTTGHLIYK |
| | Cathepsin B sensitive site |
| 555 | ALSAADTKPGTTGSGAGSGGPGGLTSAAPAGGDKK |
| | Cathepsin B sensitive site |
| 556 | STTTGHLIYK |
| | Cathepsin B sensitive site |
| 557 | VTIIGPATVGGIKPGCFK |
| | Cathepsin B sensitive site |
| 558 | TVVFSHPPIGTVGLTEDEAIHK |
| | Cathepsin B sensitive site |
| 559 | GSPTSLGTWGSWIGPDHDK |
| | Cathepsin B sensitive site |
| 560 | GSPTSLGTWGSWIGPDHDKF |
| | Cathepsin B sensitive site |
| 561 | IHFPLATYAPVISAEK |
| | Cathepsin B sensitive site |
| 562 | ANPQVGVAFPHIK |
| | Cathepsin B sensitive site |
| 563 | TCTTVAFTQVNSEDK |
| | Cathepsin B sensitive site |
| 564 | VLTGVAGEDAECHAAK |
| | Cathepsin B sensitive site |
| 565 | NIPPYFVALVPQEEELDDQK |
| | Cathepsin B sensitive site |
| 566 | GQETAVAPSLVAPALNKPK |
| | Cathepsin B sensitive site |
| 567 | QGQETAVAPSLVAPALNKPK |
| | Cathepsin B sensitive site |
| 568 | GFVTFSSMAEVDAAMAARPH |
| | Cathepsin B sensitive site |
| 569 | VDYYTTTPALVFGKPVR |
| | Cathepsin B sensitive site |
| 570 | VDYYTTTPALVFGKPVR |
| | Cathepsin B sensitive site |
| 571 | ASQPXVDGFLVGGASLKPEFVDIINAK |
| | Cathepsin B sensitive site |
| 572 | TIIGPATVGGIKPGCFK |
| | Cathepsin B sensitive site |
| 573 | GGVDTAAVGGVFDVSNADR |
| | Cathepsin B sensitive site |
| 574 | TTVHAITATQK |
| | Cathepsin B sensitive site |
| 575 | EEVRPQDTVSVIGGVAGGSK |
| | Cathepsin B sensitive site |
| 576 | QVIGTGSFFPK |
| | Cathepsin B sensitive site |
| 577 | ASGNYATVISHNPETK |
| | Cathepsin B sensitive site |
| 578 | MKPLMGVIYVPLTDKEK |
| | Cathepsin B sensitive site |
| 579 | FSVCVLGDQQHCDEAK |
| | Cathepsin B sensitive site |
| 580 | ENAFCNLAAIVPDSVGRHSPA |
| | Cathepsin B sensitive site |
| 581 | AYVGNLPFNTVQGDIDAIFK |
| | Cathepsin B sensitive site |
| 582 | TLTTVQGIADDYDKK |
| | Cathepsin B sensitive site |
| 583 | CISXHVGQAGVQIGNACWE |
| | Cathepsin B sensitive site |
| 584 | THALQWPSLTVQWLPEVTKPEGK |
| | Cathepsin B sensitive site |
| 585 | ASVPAGGAVAVSAAPGSAAPAAGSAPAAAEEK |
| | Cathepsin B sensitive site |
| 586 | YEEVSVSGFEEFHR |
| | Cathepsin B sensitive site |
| 587 | CMTTVSWDGDKLQCVQK |
| | Cathepsin B sensitive site |
| 588 | MHGGGPTVTAGLPLPK |
| | Cathepsin B sensitive site |
| 589 | LALVTGGEIASTFDHPELVK |
| | Cathepsin B sensitive site |
| 590 | LEGTLLKPNMVTPGHACTQK |
| | Cathepsin B sensitive site |
| 591 | XVVESAYEVIK |
| | Cathepsin B sensitive site |
| 592 | ILAQVVGDVDTSLPR |
| | Cathepsin B sensitive site |
| 593 | CFSEMAPVCAVVGGILAQEIVK |
| | Cathepsin B sensitive site |
| 594 | ETEDTFXADLVVGLCTGQIK |
| | Cathepsin B sensitive site |
| 595 | EGPAVVGQFIQDVK |
| | Cathepsin B sensitive site |
| 596 | MLISGYALNCVVGSQGMPK |
| | Cathepsin B sensitive site |
| 597 | HWPFMVVNDAGRPK |
| | Cathepsin B sensitive site |
| 598 | SGPVVAMVWEGLNVVK |
| | Cathepsin B sensitive site |
| 599 | ALQDEWDAVMLHSFTLRQ |
| | Cathepsin B sensitive site |
| 600 | EYFSWEGAFQHVGK |
| | Cathepsin B sensitive site |
| 601 | ATVASGIPAGWMGLDCGPESSK |
| | Cathepsin B sensitive site |
| 602 | ATVASGIPAGWMGLDCGPESSKK |
| | Cathepsin B sensitive site |
| 603 | DCAFYDPTHAWSGGLDHQLK |
| | Cathepsin B sensitive site |
| 604 | QFQALLQYADPVSAQHAK |
| | Cathepsin B sensitive site |
| 605 | EQPQHPLHVTYAGAAVDELGK |
| | Cathepsin B sensitive site |
| 606 | TFSYAGFEMQPK |
| | Cathepsin B sensitive site |
| 607 | GYIWNYGAIPQTWEDPGHNDK |
| | Cathepsin B sensitive site |
| 608 | DYTGYNNYYGYGDYSNQQSGYGK |
| | Cathepsin B sensitive site |
| 609 | QSGYGGQTKPIFR |
| | Cathepsin B sensitive site |
| 610 | VPLIESGTAGYLGQVTTIKK |
| | Cathepsin B sensitive site |
| 611 | GILGYTEHQVVSSDFNSDTH |
| | Cathepsin B sensitive site |
| 612 | GILGYTEHQVVSSDFNSDTHSS |
| | Cathepsin B sensitive site |
| 613 | QTCVXHYTGMLEDGKK |
| | Cathepsin B sensitive site |
| 614 | QTCVXHYTGMLEDGKKFDS |
| | Cathepsin B sensitive site |
| 615 | AXYVTHLMK |
| | Cathepsin B sensitive site |
| 616 | KVSVR |
| | Cathepsin S sensitive site |
| 617 | TVGLR |
| | Cathepsin S sensitive site |
| 618 | PMGLP |
| | Cathepsin S sensitive site |
| 619 | PMGAP |
| | Cathepsin S sensitive site |
| 620 | MDLAAAAEPGAGSQHLEVR |
| | Cathepsin S sensitive site |
| 621 | EGAMVAATQGAAAAAGSGAGTGGGTASGGTEGGSAESEGAK |
| | Cathepsin S sensitive site |
| 622 | GTSFDAAATSGGSASSEK |
| | Cathepsin S sensitive site |
| 623 | AMVAATQGAAAAAGSGAGTGGGTASGGTEGGSAESEGAK |
| | Cathepsin S sensitive site |
| 624 | GILAADESTGSIAK |
| | Cathepsin S sensitive site |
| 625 | PAAPALSAADTKPGTTGSGAGSGGPGGLT |
| | Cathepsin S sensitive site |
| 626 | MVAATQGAAAAAGSGAGTGGGTASGGTEGGSAESEGAK |
| | Cathepsin S sensitive site |
| 627 | SSIQATTAAGSGHPTSCC |
| | Cathepsin S sensitive site |
| 628 | NEAIQAAHDAVAQEGQCR |
| | Cathepsin S sensitive site |
| 629 | QFGLPAEAVEAANKGDVEAFAK |
| | Cathepsin S sensitive site |
| 630 | LVIPNTLAVNAAQDSTDLVAK |
| | Cathepsin S sensitive site |
| 631 | EALAAMNAAQVKPLGK |
| | Cathepsin S sensitive site |
| 632 | APRPPVSAASGRPQDDTDSSR |
| | Cathepsin S sensitive site |
| 633 | GDPQEAKPQEAAVAPEKPPASDETK |
| | Cathepsin S sensitive site |
| 634 | EGDMIVCAAYAHELPK |
| | Cathepsin S sensitive site |
| 635 | GILAADESTGSIAK |
| | Cathepsin S sensitive site |
| 636 | PEEACSFILSADFPALVVK |
| | Cathepsin S sensitive site |
| 637 | GWNAYIDNLMADGTCQDAAIVGYK |
| | Cathepsin S sensitive site |
| 638 | YLAADKDGNVTCER |
| | Cathepsin S sensitive site |
| 639 | LPVDFVTADKFDENAK |
| | Cathepsin S sensitive site |
| 640 | TXEAEAAHGTVTR |
| | Cathepsin S sensitive site |
| 641 | TVFAEHISDECK |
| | Cathepsin S sensitive site |
| 642 | TVFAEHISDECKR |
| | Cathepsin S sensitive site |
| 643 | DLEAEHVEVEDTTLNR |
| | Cathepsin S sensitive site |
| 644 | CAEIAHNVSSK |
| | Cathepsin S sensitive site |
| 645 | EAAAAGGGVGAGAGGGCGPGGADSSKPR |
| | Cathepsin S sensitive site |
| 646 | YXLVGAGAIGCELLK |
| | Cathepsin S sensitive site |
| 647 | LIYAGKILNDDTALK |
| | Cathepsin S sensitive site |
| 648 | FGDNTAGCTSAGPHFNPLSR |
| | Cathepsin S sensitive site |
| 649 | IITLAGPTNAIFK |
| | Cathepsin S sensitive site |
| 650 | EIVHXQAGQCGNQIGAK |
| | Cathepsin S sensitive site |
| 651 | AICAGPTALLAHEIGFGSK |
| | Cathepsin S sensitive site |
| 652 | SHEHSPSDLEAHFVPLVK |
| | Cathepsin S sensitive site |
| 653 | ELQAHGADELLK |
| | Cathepsin S sensitive site |
| 654 | SWADLVNAHVVPGSGVVK |
| | Cathepsin S sensitive site |
| 655 | TFIAIKPDGVQR |
| | Cathepsin S sensitive site |
| 656 | GYIWNYGAIPQTWEDPGHNDK |
| | Cathepsin S sensitive site |
| 657 | ATATXXAKPQITNPK |
| | Cathepsin S sensitive site |
| 658 | TTETAQHAQGAKPQVQPQK |
| | Cathepsin S sensitive site |
| 659 | VASYLLAALGGNSSPSAK |
| | Cathepsin S sensitive site |
| 660 | IALPAPRGSGTASD |
| | Cathepsin S sensitive site |
| 661 | QIGNVAALPGIVHR |
| | Cathepsin S sensitive site |
| 662 | DGTVLCELINALYPEGQAPVK |
| | Cathepsin S sensitive site |
| 663 | DGTVLCELINALYPEGQAPVKK |
| | Cathepsin S sensitive site |
| 664 | DFTVSAMHGDMDQK |
| | Cathepsin S sensitive site |
| 665 | LVTDCVAAMNPDAVLR |
| | Cathepsin S sensitive site |
| 666 | HELQANCYEEVKDR |
| | Cathepsin S sensitive site |
| 667 | CSLQAAAILDANDAHQTETSSSQVK |
| | Cathepsin S sensitive site |
| 668 | SGLGRPQLQGAPAAEPMAVP |
| | Cathepsin S sensitive site |
| 669 | QETAVAPSLVAPALNKPK |
| | Cathepsin S sensitive site |
| 670 | AQXAAPASVPAQAPK |
| | Cathepsin S sensitive site |
| 671 | GETIFVTAPHEATAGIIGVNR |
| | Cathepsin S sensitive site |
| 672 | EYSSELNAPSQESDSHPR |
| | Cathepsin S sensitive site |
| 673 | DQVTAQEIFQDNHEDGPTAK |
| | Cathepsin S sensitive site |
| 674 | LHEEEIQELQAQIQEQHVQ |
| | Cathepsin S sensitive site |
| 675 | QQQRPLEAQPSAPGHSVK |
| | Cathepsin S sensitive site |
| 676 | AAHTANFLLNASGSTSTPAPSR |
| | Cathepsin S sensitive site |
| 677 | IXXXFLGASLKDEVLK |
| | Cathepsin S sensitive site |
| 678 | NVEEADAAMAASPHAVDGNTVELK |
| | Cathepsin S sensitive site |
| 679 | ALLVTASQCQQPAENK |
| | Cathepsin S sensitive site |
| 680 | QSSWGMMGMLASQQNQSGPSGNNQNQGNMQR |
| | Cathepsin S sensitive site |
| 681 | LPPGFSASSTVEKPSK |
| | Cathepsin S sensitive site |
| 682 | AAVPSGASTGIYEALE |
| | Cathepsin S sensitive site |
| 683 | LNCQVIGASVDSHFCH |
| | Cathepsin S sensitive site |
| 684 | GANQYTFHLEATENPGALIK |
| | Cathepsin S sensitive site |
| 685 | LSELTQQLAQATGKPPQYIAVH |
| | Cathepsin S sensitive site |
| 686 | AGEQEGAMVAATQGAAAAAGSGAGTGGGTASGGTEGGSAESEGAK |
| | Cathepsin S sensitive site |
| 687 | ISSIQATTAAGSGHPTSCC |
| | Cathepsin S sensitive site |
| 688 | GLGATTHPTAAVK |
| | Cathepsin S sensitive site |
| 689 | IEPPPLDAVIEAEHTLR |
| | Cathepsin S sensitive site |
| 690 | SXGLPVGAVINCADNTGAK |
| | Cathepsin S sensitive site |
| 691 | VNVANVGAVPSGQDNIHR |
| | Cathepsin S sensitive site |
| 692 | QFLECAQNQGDIK |
| | Cathepsin S sensitive site |
| 693 | GGLTDEAALSCCSDADPSTK |
| | Cathepsin S sensitive site |
| 694 | ILSCGEVIHVK |
| | Cathepsin S sensitive site |
| 695 | AIVDCGFEHPSEVQ |
| | Cathepsin S sensitive site |
| 696 | HYYEVSCHDQGLCR |
| | Cathepsin S sensitive site |
| 697 | MLVQCMQDQEHPSIR |
| | Cathepsin S sensitive site |
| 698 | DVVICPDASLEDAKK |
| | Cathepsin S sensitive site |
| 699 | SGYAFVDCPDEHWAMK |
| | Cathepsin S sensitive site |
| 700 | FVLCPECENPETDLHVNPK |
| | Cathepsin S sensitive site |
| 701 | IAILTCPFEPPKPK |
| | Cathepsin S sensitive site |
| 702 | IAILTCPFEPPKPK |
| | Cathepsin S sensitive site |
| 703 | AATEQYHQVLCPGPSQDDPLHPLNK |
| | Cathepsin S sensitive site |
| 704 | AIVICPTDEDLKDR |
| | Cathepsin S sensitive site |
| 705 | ATAHAQAQLGCPVIIHPGR |
| | Cathepsin S sensitive site |
| 706 | IIPGXMCQGGDFTR |
| | Cathepsin S sensitive site |
| 707 | IIPGXMCQGGDFTR |
| | Cathepsin S sensitive site |
| 708 | PALYWLSGLTCTEQNFISK |
| | Cathepsin S sensitive site |
| 709 | MTVGCVAGDEESYEVFK |
| | Cathepsin S sensitive site |
| 710 | ATVAFCDAQSTQEIHEK |
| | Cathepsin S sensitive site |
| 711 | NYGILADATEQVGQHK |
| | Cathepsin S sensitive site |
| 712 | LQDCEGLIVR |
| | Cathepsin S sensitive site |
| 713 | QISAGYXPVXDCHTAHIACK |
| | Cathepsin S sensitive site |
| 714 | QISAGYXPVXDCHTAHIACK |
| | Cathepsin S sensitive site |
| 715 | TGEPCCDWVGDEGAGHFVK |
| | Cathepsin S sensitive site |
| 716 | DATNVGDEGGFAPNILENK |
| | Cathepsin S sensitive site |
| 717 | NILDFPQHVSPSK |
| | Cathepsin S sensitive site |
| 718 | DHVVSDFSEHGSLK |
| | Cathepsin S sensitive site |
| 719 | ADNELSPECLDGAQHFLK |
| | Cathepsin S sensitive site |
| 720 | IQTLGYFPVGDGDFPHQK |
| | Cathepsin S sensitive site |
| 721 | DXQEXXXFLLDGLHEDLNR |
| | Cathepsin S sensitive site |
| 722 | MILIQDGSQNTNVDKPLR |
| | Cathepsin S sensitive site |
| 723 | FTISDHPQPIDPLLK |
| | Cathepsin S sensitive site |
| 724 | VNPTXFFDIAVDGEPLGR |
| | Cathepsin S sensitive site |
| 725 | YEDICPSTHNMDVPNIK |
| | Cathepsin S sensitive site |
| 726 | NGSIYNPEVLDITEETLHSR |
| | Cathepsin S sensitive site |
| 727 | QHIVNDMNPGNLH |
| | Cathepsin S sensitive site |
| 728 | MLLDSEQHPCQLK |
| | Cathepsin S sensitive site |
| 729 | EGLMLDSHEELYK |
| | Cathepsin S sensitive site |
| 730 | TAGDTHLGGEDFDNR |
| | Cathepsin S sensitive site |
| 731 | PGGLLLGDVAPNFEANTTVGR |
| | Cathepsin S sensitive site |
| 732 | NDGATILSMMDVDHQIAK |
| | Cathepsin S sensitive site |
| 733 | GEEGLTLNLEDVQPHDLGK |
| | Cathepsin S sensitive site |
| 734 | TIDNSQGAYQEAFDISKK |
| | Cathepsin S sensitive site |
| 735 | IVGFFDDSFSEAHSEFLK |
| | Cathepsin S sensitive site |
| 736 | QEEASGVALGEAPDHSYESLR |
| | Cathepsin S sensitive site |
| 737 | DPVQEAWAEDVDLR |
| | Cathepsin S sensitive site |
| 738 | PMIYICGECHTENEIK |
| | Cathepsin S sensitive site |
| 739 | HMSEFMECNLNELVK |
| | Cathepsin S sensitive site |
| 740 | MGYAEEAPYDAIHVG |
| | Cathepsin S sensitive site |
| 741 | MADQLTEEQIAEFK |
| | Cathepsin S sensitive site |
| 742 | YLAEFATGNDRK |
| | Cathepsin S sensitive site |
| 743 | LAELEEFINGPNNAHIQ |
| | Cathepsin S sensitive site |
| 744 | XEFTDHLVK |
| | Cathepsin S sensitive site |
| 745 | FCVGFLEGGKDSCQGDSGGPVVC |
| | Cathepsin S sensitive site |
| 746 | LLAEGHPDPDAELQR |
| | Cathepsin S sensitive site |
| 747 | GLTEGLHGFH |
| | Cathepsin S sensitive site |
| 748 | FVHWYVGEGMEEGEFSEAR |
| | Cathepsin S sensitive site |
| 749 | MLLHEGQHPAQLR |
| | Cathepsin S sensitive site |
| 750 | YHGYTFANLGEHEFVEEK |
| | Cathepsin S sensitive site |
| 751 | TVFAEHISDECK |
| | Cathepsin S sensitive site |
| 752 | VILEEHSTCENEVSK |
| | Cathepsin S sensitive site |
| 753 | LLTEIHGGAGGPSGR |
| | Cathepsin S sensitive site |
| 754 | AHLMEIQVNGGTVAEK |
| | Cathepsin S sensitive site |
| 755 | ISWLDANTLAEKDEFEHK |
| | Cathepsin S sensitive site |
| 756 | XEKFEDENFILK |
| | Cathepsin S sensitive site |
| 757 | SAVEAGSEVSEKPGQEAPVLPK |
| | Cathepsin S sensitive site |
| 758 | ILNEKPTTDEPEK |
| | Cathepsin S sensitive site |
| 759 | YLAEKYEWDVAEAR |
| | Cathepsin S sensitive site |
| 760 | CLELFXELAEDKENY |
| | Cathepsin S sensitive site |
| 761 | CLELFXELAEDKENYK |
| | Cathepsin S sensitive site |
| 762 | MEELHNQEVQK |
| | Cathepsin S sensitive site |
| 763 | GVNVAGVSLQELNPEMGTDNDSENWK |
| | Cathepsin S sensitive site |
| 764 | ASDIAMTELPPTHPIR |
| | Cathepsin S sensitive site |
| 765 | VVVAENFDEIVNNENK |
| | Cathepsin S sensitive site |
| 766 | IXEGCEEPATHNALAK |
| | Cathepsin S sensitive site |
| 767 | VTEQGAELSNEER |
| | Cathepsin S sensitive site |
| 768 | AVTEQGHELSNEER |
| | Cathepsin S sensitive site |
| 769 | QVDQEEPHVEEQQQQTPAENK |
| | Cathepsin S sensitive site |
| 770 | VVFEQTKVIADNVK |
| | Cathepsin S sensitive site |
| 771 | NIFVGENILEESENLHNADQPLR |
| | Cathepsin S sensitive site |
| 772 | LFIHESIHDEVVNR |
| | Cathepsin S sensitive site |
| 773 | VTNGIEEPLEESSHEPEPEPESETK |
| | Cathepsin S sensitive site |
| 774 | GIVEESVTGVHR |
| | Cathepsin S sensitive site |
| 775 | QCPSVVSLLSESYNPHVR |
| | Cathepsin S sensitive site |
| 776 | ASLQETHFDSTQTK |
| | Cathepsin S sensitive site |
| 777 | TFGETHPFTK |
| | Cathepsin S sensitive site |
| 778 | VMLGETNPADSKPGTIR |
| | Cathepsin S sensitive site |
| 779 | GADFLVTEVENGGSLGSK |
| | Cathepsin S sensitive site |
| 780 | LPTEAYISVEEVHDDGTPTSK |
| | Cathepsin S sensitive site |
| 781 | MEEVPHDCPGADSAQAGR |
| | Cathepsin S sensitive site |
| 782 | VDENCVGFDHTVKPV |
| | Cathepsin S sensitive site |
| 783 | VHVVPDQLMAFGGSSEPCALC |
| | Cathepsin S sensitive site |
| 784 | IWCFGPDGTGPNILT |
| | Cathepsin S sensitive site |
| 785 | YVXFGPHAGK |
| | Cathepsin S sensitive site |
| 786 | EFAGFQCQIQFGPHNEQK |
| | Cathepsin S sensitive site |
| 787 | KPXKPMQFLGDEETVRK |
| | Cathepsin S sensitive site |
| 788 | MVYMFQYDSTHGK |
| | Cathepsin S sensitive site |
| 789 | EELGFRPEYSASQLK |
| | Cathepsin S sensitive site |
| 790 | HLEFSHDQYR |
| | Cathepsin S sensitive site |
| 791 | TCGFDFTGAVEDISK |
| | Cathepsin S sensitive site |
| 792 | GFGFVDFNSEEDAK |
| | Cathepsin S sensitive site |
| 793 | NYGFVHIEDK |
| | Cathepsin S sensitive site |
| 794 | GFGFVTFDDHDPVDK |
| | Cathepsin S sensitive site |
| 795 | LPNFGFVVFDDSEPVQK |
| | Cathepsin S sensitive site |
| 796 | QLLCGAAIGTHEDDK |
| | Cathepsin S sensitive site |
| 797 | QLLCGAAIGTHEDDKYR |
| | Cathepsin S sensitive site |
| 798 | MTNGFSGADLTEICQR |
| | Cathepsin S sensitive site |
| 799 | VQGEVMEGADNQGAGEQGRPVR |
| | Cathepsin S sensitive site |
| 800 | MGGHGYGGAGDASSGFHGGHF |
| | Cathepsin S sensitive site |
| 801 | LGNVLGGLISGAGGGGGGGGGGGGGGGGGGGGTAMR |
| | Cathepsin S sensitive site |
| 802 | FGGSFAGSFGGAGGHAPGVAR |
| | Cathepsin S sensitive site |
| 803 | VLVVGAGGIGCELLK |
| | Cathepsin S sensitive site |
| 804 | VTADHGPAVSGAHNTIICAR |
| | Cathepsin S sensitive site |
| 805 | CEALAGAPLDNAPK |
| | Cathepsin S sensitive site |
| 806 | STGGAPTFNVTVTK |
| | Cathepsin S sensitive site |
| 807 | KGCDVVVIPAGVPR |
| | Cathepsin S sensitive site |
| 808 | FSPAGVEGCPALPHK |
| | Cathepsin S sensitive site |
| 809 | HSSLAGCQIINYR |
| | Cathepsin S sensitive site |
| 810 | SSEVGYDAMAGDFVNMVEK |
| | Cathepsin S sensitive site |
| 811 | SIEDSVISLSGDHCIIGR |
| | Cathepsin S sensitive site |
| 812 | VTGDHIPTPQDLPQR |
| | Cathepsin S sensitive site |
| 813 | NGDTFLGGEDFDQALLR |
| | Cathepsin S sensitive site |
| 814 | IVYICCGEDHTAALTK |
| | Cathepsin S sensitive site |
| 815 | MVDGNVSGEFTDLVPEK |
| | Cathepsin S sensitive site |
| 816 | MAAQGEPQVQFK |
| | Cathepsin S sensitive site |
| 817 | QALAVHLALQGESSSEHFLK |
| | Cathepsin S sensitive site |
| 818 | AFYNNVLGEYEEYITK |
| | Cathepsin S sensitive site |
| 819 | LLNQMDGFDTLHR |
| | Cathepsin S sensitive site |
| 820 | GLTEGLHGFHVHEFG |
| | Cathepsin S sensitive site |
| 821 | GLTEGLHGFHVHEFGDNTAGCT |
| | Cathepsin S sensitive site |
| 822 | AADSYFSLLQGFINSLDESTQESK |
| | Cathepsin S sensitive site |
| 823 | INPYLLGTMAGGAADCSFWER |
| | Cathepsin S sensitive site |
| 824 | QHDLFDSGFGGGAGVETGGK |
| | Cathepsin S sensitive site |
| 825 | TTHFVEGGDAGNREDQINR |
| | Cathepsin S sensitive site |
| 826 | SQPIAQQPLQGGDHSGNYGYK |
| | Cathepsin S sensitive site |
| 827 | GTDGTDNPLSGGDQYQNITVHR |
| | Cathepsin S sensitive site |
| 828 | GCITXIGGGDTATCCAK |
| | Cathepsin S sensitive site |
| 829 | WGSGGGGGGGGGGGGGGGGGGGGGGGGGGGRKSSSAAA |
| | Cathepsin S sensitive site |
| 830 | LAAGSLAAPGGGGGSAGGARP |
| | Cathepsin S sensitive site |
| 831 | GSXXXGGGSYNDFGNY |
| | Cathepsin S sensitive site |
| 832 | VNAANXSLLGGGGVDGCIHR |
| | Cathepsin S sensitive site |
| 833 | FCVGFLEGGKDSCQGDSGGPVVC |
| | Cathepsin S sensitive site |
| 834 | LVDGQIFCLHGGLSPSIDTLDHIR |
| | Cathepsin S sensitive site |
| 835 | MFXGGLSWDTSKK |
| | Cathepsin S sensitive site |
| 836 | DPQELLEGGNQGEGDPQAEGR |
| | Cathepsin S sensitive site |
| 837 | NMGGPYGGGNYGPGGSGGSGGYGGR |
| | Cathepsin S sensitive site |
| 838 | RGGPGGPGGPGGPMGR |
| | Cathepsin S sensitive site |
| 839 | SVLDDWFPLQGGQGQVHLR |
| | Cathepsin S sensitive site |
| 840 | IIMEYLGGGSALDLLR |
| | Cathepsin S sensitive site |
| 841 | SHFAMMHGGTGFAGIDSSSPEVK |
| | Cathepsin S sensitive site |
| 842 | QGFQLTHSLGGGTGSGMGTLLI |
| | Cathepsin S sensitive site |
| 843 | MADYLISGGTSYVPDDGLT |
| | Cathepsin S sensitive site |
| 844 | VTVAGGVHISGLH |
| | Cathepsin S sensitive site |
| 845 | VTVAGGVHISGLHT |
| | Cathepsin S sensitive site |
| 846 | VTVAGGVHISGLHTE |
| | Cathepsin S sensitive site |
| 847 | YAVSELAGHQTSAESWGTGR |
| | Cathepsin S sensitive site |
| 848 | TFQGHTNEVNAIK |
| | Cathepsin S sensitive site |
| 849 | GDGPVQGIINFEQK |
| | Cathepsin S sensitive site |
| 850 | VTIIGPATVGGIKPGCFK |
| | Cathepsin S sensitive site |
| 851 | FSLPGMEHVYGIPEHADNLR |
| | Cathepsin S sensitive site |
| 852 | LPPSGAVPVTGIPPHVVK |
| | Cathepsin S sensitive site |
| 853 | MDGIVPDIAVGTK |
| | Cathepsin S sensitive site |
| 854 | RGIWHNDNK |
| | Cathepsin S sensitive site |
| 855 | GKPEIEGKPESEGEPGSETR |
| | Cathepsin S sensitive site |
| 856 | YDINAHACVTGKPISQGGIHGR |
| | Cathepsin S sensitive site |
| 857 | ELTQQLAQATGKPPQYIAVH |
| | Cathepsin S sensitive site |
| 858 | NPKPFLNGLTGKPVMVK |
| | Cathepsin S sensitive site |
| 859 | CPSILGGLAPEKDQPK |
| | Cathepsin S sensitive site |
| 860 | VASGIPAGWXGLDCGPESSKK |
| | Cathepsin S sensitive site |
| 861 | QVLQGLDYLHSK |
| | Cathepsin S sensitive site |
| 862 | GALEGLPRPPPPVK |
| | Cathepsin S sensitive site |
| 863 | LFIGGLSFETTDESLR |
| | Cathepsin S sensitive site |
| 864 | VFVGGLSPDTSEEQIK |
| | Cathepsin S sensitive site |
| 865 | MFXGGLSWDTSKK |
| | Cathepsin S sensitive site |
| 866 | NVIIWGNHSSTQYPDVNHAK |
| | Cathepsin S sensitive site |
| 867 | LLSGLAEGLGGNIEQLVAR |
| | Cathepsin S sensitive site |
| 868 | LVINGNPITIFQER |
| | Cathepsin S sensitive site |
| 869 | SAAMLGNSEDHTALSR |
| | Cathepsin S sensitive site |
| 870 | IFQGNVHNFEK |
| | Cathepsin S sensitive site |
| 871 | NNPPTLEGNYSKPLK |
| | Cathepsin S sensitive site |
| 872 | MVGPAVIVDKK |
| | Cathepsin S sensitive site |
| 873 | MMLGPEGGEGFVVK |
| | Cathepsin S sensitive site |
| 874 | SIYEALGGPHDPNVAK |
| | Cathepsin S sensitive site |
| 875 | TFQGPNCPATCGR |
| | Cathepsin S sensitive site |
| 876 | IMGPNYTPGKK |
| | Cathepsin S sensitive site |
| 877 | MVIITGPPEAQFK |
| | Cathepsin S sensitive site |
| 878 | AFGLTDDQVSGPPSAPAEDR |
| | Cathepsin S sensitive site |
| 879 | TVQGPPTSDDIFER |
| | Cathepsin S sensitive site |
| 880 | FVIGGPQGDAGLTGR |
| | Cathepsin S sensitive site |
| 881 | IITLXGPTNAIFK |
| | Cathepsin S sensitive site |
| 882 | KPPTLIHGQAPSAGLPSQKPK |
| | Cathepsin S sensitive site |
| 883 | RGQGGYPGKPR |
| | Cathepsin S sensitive site |
| 884 | RPDNFXFGQSGAGNNWAK |
| | Cathepsin S sensitive site |
| 885 | GLLALSSALSGQSHLAIK |
| | Cathepsin S sensitive site |
| 886 | ALPPVLTTVNGQSPPEHSAPAK |
| | Cathepsin S sensitive site |
| 887 | QSGYGGQTKPIFR |
| | Cathepsin S sensitive site |
| 888 | LSGQTNIHLSK |
| | Cathepsin S sensitive site |
| 889 | VVLMSHLGRPDGVPMPDK |
| | Cathepsin S sensitive site |
| 890 | VVLMSHLGRPDGVPMPDKY |
| | Cathepsin S sensitive site |
| 891 | QQSIAGSADSKPIDVSR |
| | Cathepsin S sensitive site |
| 892 | VTLGPVPEIGGSEAPAPQNK |
| | Cathepsin S sensitive site |
| 893 | NFGGSFAGSFGGAGGHAPGVAR |
| | Cathepsin S sensitive site |
| 894 | MMDYLQGSGETPQTDVR |
| | Cathepsin S sensitive site |
| 895 | DSVWGSGGGQQSVNHLVK |
| | Cathepsin S sensitive site |
| 896 | PQVAIICGSGLGGLTDK |
| | Cathepsin S sensitive site |
| 897 | PTSSEQGGLEGSGSAAGEGKPALSEEER |
| | Cathepsin S sensitive site |
| 898 | TVEQLLTGSPTSPTVEPEKPTR |
| | Cathepsin S sensitive site |
| 899 | GCLEGSQGTQALHK |
| | Cathepsin S sensitive site |
| 900 | LLAVSAPALQGSRPGETEENVR |
| | Cathepsin S sensitive site |
| 901 | IXXGSSGAQGSGGGSTSAHY |
| | Cathepsin S sensitive site |
| 902 | VAFTGSTEVGHLIQK |
| | Cathepsin S sensitive site |
| 903 | VVVLMGSTSDLGHCEK |
| | Cathepsin S sensitive site |
| 904 | MVELLGSYTEDNASQAR |
| | Cathepsin S sensitive site |
| 905 | IYWGTATTGKPHVA |
| | Cathepsin S sensitive site |
| 906 | IVGFCWGGTAVHHLM |
| | Cathepsin S sensitive site |
| 907 | GVVPLAGTDGETTTQGLDGLSER |
| | Cathepsin S sensitive site |
| 908 | GXVXFXGTDHIDQWNK |
| | Cathepsin S sensitive site |
| 909 | SVSGTDVQEECR |
| | Cathepsin S sensitive site |
| 910 | MMLGTEGGEGFVVK |
| | Cathepsin S sensitive site |
| 911 | IAFHQDGSLAGTGGLDAFGR |
| | Cathepsin S sensitive site |
| 912 | LNFSHGTHEYHAETIK |
| | Cathepsin S sensitive site |
| 913 | LVLGTHTSDEQNHLV |
| | Cathepsin S sensitive site |
| 914 | ALHWLVLGTHTSDEQNHLVVAR |
| | Cathepsin S sensitive site |
| 915 | VLSGTIHAGQPVK |
| | Cathepsin S sensitive site |
| 916 | IITITGTQDQIQNAQY |
| | Cathepsin S sensitive site |
| 917 | GGTSDVEVNEK |
| | Cathepsin S sensitive site |
| 918 | VLTGVAGEDAECHAAK |
| | Cathepsin S sensitive site |
| 919 | TGGVDTAAVGGVFDVSNADR |
| | Cathepsin S sensitive site |
| 920 | FIVDGWHEMDAENPLH |
| | Cathepsin S sensitive site |
| 921 | TMFSSEVQFGHAGACANQASETAVAK |
| | Cathepsin S sensitive site |
| 922 | PIYDVLQMVGHANRPLQDDEGR |
| | Cathepsin S sensitive site |
| 923 | EWAHATIIPK |
| | Cathepsin S sensitive site |
| 924 | KHEANNPQLK |
| | Cathepsin S sensitive site |
| 925 | MVNHFIAEFK |
| | Cathepsin S sensitive site |
| 926 | LVXHFVEEFK |
| | Cathepsin S sensitive site |
| 927 | MPFPVNHGASSEDTLLK |
| | Cathepsin S sensitive site |
| 928 | NXCWELYCLEHGIQPDGQMPSDK |
| | Cathepsin S sensitive site |
| 929 | NXCWELYCLEHGIQPDGQMPSDK |
| | Cathepsin S sensitive site |
| 930 | VHAGPFANIAHGNSSIIADR |
| | Cathepsin S sensitive site |
| 931 | INQVFHGSCITEGNELTK |
| | Cathepsin S sensitive site |
| 932 | FELQHGTEEQQEEVR |
| | Cathepsin S sensitive site |
| 933 | EQQEAIEHIDEVQNEIDR |
| | Cathepsin S sensitive site |
| 934 | AVEALAAALAHISGATSVDQR |
| | Cathepsin S sensitive site |
| 935 | RHLAPTGNAPASR |
| | Cathepsin S sensitive site |
| 936 | LLTDFCTHLPNLPDSTAK |
| | Cathepsin S sensitive site |
| 937 | VDEFVTHNLSFDEINK |
| | Cathepsin S sensitive site |
| 938 | ATLELTHNWGTEDDETQSY |
| | Cathepsin S sensitive site |
| 939 | EEFTAFLHPEEYDYMK |
| | Cathepsin S sensitive site |
| 940 | QXFHPEQLITGK |
| | Cathepsin S sensitive site |
| 941 | PVTHNLPTVAHPSQAPSPNQPTK |
| | Cathepsin S sensitive site |
| 942 | AXXXXXQHQAGQAPHLG |
| | Cathepsin S sensitive site |
| 943 | CNFTDGALVQHQEWDGK |
| | Cathepsin S sensitive site |
| 944 | GVLHQFSGTETNK |
| | Cathepsin S sensitive site |
| 945 | QIGAVVSHQSSVIPDR |
| | Cathepsin S sensitive site |
| 946 | IEPNEVTHSGDTGVETDGR |
| | Cathepsin S sensitive site |
| 947 | HYAHTDCPGHADYVK |
| | Cathepsin S sensitive site |
| 948 | TICSHVQNMIK |
| | Cathepsin S sensitive site |
| 949 | LLGHWEEAAHDLA |
| | Cathepsin S sensitive site |
| 950 | TYTIANQFPLNK |
| | Cathepsin S sensitive site |
| 951 | NPTXFFDIAVDGEPLGR |
| | Cathepsin S sensitive site |
| 952 | LVSIGAEEIVDGNAK |
| | Cathepsin S sensitive site |
| 953 | TTDGVYEGVAIGGDRYPGSTF |
| | Cathepsin S sensitive site |
| 954 | THINIVVIGHVDSGK |
| | Cathepsin S sensitive site |
| 955 | DNDFCGTDMTIGTDSALHR |
| | Cathepsin S sensitive site |
| 956 | VLXNMEIGTSLFDEEGAK |
| | Cathepsin S sensitive site |
| 957 | VCTLAIIDPGDSDIIR |
| | Cathepsin S sensitive site |
| 958 | GCITIIGGGDTATCCAK |
| | Cathepsin S sensitive site |
| 959 | TFNQVEIKPEMIGH |
| | Cathepsin S sensitive site |
| 960 | CQLEINFNTLQTK |
| | Cathepsin S sensitive site |
| 961 | HLEINPDHPIVE |
| | Cathepsin S sensitive site |
| 962 | HLEINPDHSIIETLR |
| | Cathepsin S sensitive site |
| 963 | VPYLIAGIQHSCQDIGAK |
| | Cathepsin S sensitive site |
| 964 | VLSIQSHVIR |
| | Cathepsin S sensitive site |
| 965 | ELGITALHIK |
| | Cathepsin S sensitive site |
| 966 | LVAIVDPHIK |
| | Cathepsin S sensitive site |
| 967 | TLTIVDTGIGMTK |
| | Cathepsin S sensitive site |
| 968 | LVAIVDVIDQNR |
| | Cathepsin S sensitive site |
| 969 | QIILEKEETEELKR |
| | Cathepsin S sensitive site |
| 970 | XKHPDADSLY |
| | Cathepsin S sensitive site |
| 971 | CIGKPGGSLDNSEQK |
| | Cathepsin S sensitive site |
| 972 | HHIYLEGTLLKPNMVTPGHACTQK |
| | Cathepsin S sensitive site |
| 973 | LTQQLAQATGKPPQYIAVH |
| | Cathepsin S sensitive site |
| 974 | SSPPELPDVMKPQDSGSSANEQAVQ |
| | Cathepsin S sensitive site |
| 975 | LQELEKYPGIQTR |
| | Cathepsin S sensitive site |
| 976 | WIGLDLSNGKPR |
| | Cathepsin S sensitive site |
| 977 | MPFLELDTNLPANR |
| | Cathepsin S sensitive site |
| 978 | ETALLSSGFSLEDPQTHANR |
| | Cathepsin S sensitive site |
| 979 | EAFSLFDKDGDGTITTK |
| | Cathepsin S sensitive site |
| 980 | YELGRPAANTK |
| | Cathepsin S sensitive site |
| 981 | GNPICSLHDQGAGGNGNVLK |
| | Cathepsin S sensitive site |
| 982 | VILHLKEDQTEYLEER |
| | Cathepsin S sensitive site |
| 983 | IQQLCEDIIQLKPDVVITEK |
| | Cathepsin S sensitive site |
| 984 | IQQLCEDIIQLKPDVVITEK |
| | Cathepsin S sensitive site |
| 985 | TLNNDIMLIK |
| | Cathepsin S sensitive site |
| 986 | NQVALNPQNTVFDAK |
| | Cathepsin S sensitive site |
| 987 | NQVALNPQNTVFDAK |
| | Cathepsin S sensitive site |
| 988 | STATLAWGVNLPAHTVIIK |
| | Cathepsin S sensitive site |
| 989 | EXLELPEDEEEKK |
| | Cathepsin S sensitive site |
| 990 | GVNLPGAAVDLPAVSEK |
| | Cathepsin S sensitive site |
| 991 | RLPPAAGDEP |
| | Cathepsin S sensitive site |
| 992 | LDLPPYETF |
| | Cathepsin S sensitive site |
| 993 | DGDSVMVLPTIPEEEAKK |
| | Cathepsin S sensitive site |
| 994 | EIVHLQAGQCGNQIGAK |
| | Cathepsin S sensitive site |
| 995 | DVSIEDSVISLSGDHCIIGR |
| | Cathepsin S sensitive site |
| 996 | SSAPGPLELDLTGDLESFKK |
| | Cathepsin S sensitive site |
| 997 | FLEMCNDLLAR |
| | Cathepsin S sensitive site |
| 998 | TTGFGMIYDSLDYAK |
| | Cathepsin S sensitive site |
| 999 | XMNPTNTVFDAK |
| | Cathepsin S sensitive site |
| 1000 | EDAMAMVDHCLK |
| | Cathepsin S sensitive site |
| 1001 | ANXVXSGGXTMYPGIADR |
| | Cathepsin S sensitive site |
| 1002 | ALQDLENAASGDAAVHQR |
| | Cathepsin S sensitive site |
| 1003 | DPVTNLNNAFEVAEK |
| | Cathepsin S sensitive site |
| 1004 | XNAGPNINGSQFF |
| | Cathepsin S sensitive site |
| 1005 | NYSVFYYEIQNAPEQACH |
| | Cathepsin S sensitive site |
| 1006 | ELISNASDALDKIR |
| | Cathepsin S sensitive site |
| 1007 | YYFNHITNASQWERPSGNSSSGGK |
| | Cathepsin S sensitive site |
| 1008 | TNDWEDHLAVK |
| | Cathepsin S sensitive site |
| 1009 | AFHNEAQVNPERK |
| | Cathepsin S sensitive site |
| 1010 | NCLTNFHGMDLTR |
| | Cathepsin S sensitive site |
| 1011 | TNVANFPGHSGPIT |
| | Cathepsin S sensitive site |
| 1012 | ILNNGHAFNVEFDDSQDK |
| | Cathepsin S sensitive site |
| 1013 | IEQLQNHENEDIYK |
| | Cathepsin S sensitive site |
| 1014 | PVFVHAGPFANIAHGNSSIIADR |
| | Cathepsin S sensitive site |
| 1015 | VWYVSNIDGTHIAK |
| | Cathepsin S sensitive site |
| 1016 | CDEVMQLLLENLGNENVHR |
| | Cathepsin S sensitive site |
| 1017 | QDQRPLHPVANPHAEISTK |
| | Cathepsin S sensitive site |
| 1018 | XNPLDAGAAEPI |
| | Cathepsin S sensitive site |
| 1019 | LIPQLVANVTNPNSTEHMK |
| | Cathepsin S sensitive site |
| 1020 | SAAMLGNSEDHTALSR |
| | Cathepsin S sensitive site |
| 1021 | NYQQNYQNSESGEKNEGSESAPEGQAQQR |
| | Cathepsin S sensitive site |
| 1022 | LGEMWNNTAADDKQPYEK |
| | Cathepsin S sensitive site |
| 1023 | IMQNTDPHSQEYVEHLK |
| | Cathepsin S sensitive site |
| 1024 | ILIANTGMDTDKIK |
| | Cathepsin S sensitive site |
| 1025 | AWVWNTHADFADECPKPELL |
| | Cathepsin S sensitive site |
| 1026 | DHASIQMNVAEVDKVTGR |
| | Cathepsin S sensitive site |
| 1027 | ALANVNIGSLIC |
| | Cathepsin S sensitive site |
| 1028 | EHGXXTNWDDMEK |
| | Cathepsin S sensitive site |
| 1029 | SAAQAAAQTNSNAAGK |
| | Cathepsin S sensitive site |
| 1030 | EETFEAAMLGQAEEVVQER |
| | Cathepsin S sensitive site |
| 1031 | PPYDEQTQAFIDAAQEAR |
| | Cathepsin S sensitive site |
| 1032 | LEQGQAIDDLMPAQK |
| | Cathepsin S sensitive site |
| 1033 | SLHQAIEGDTSGDFLK |
| | Cathepsin S sensitive site |
| 1034 | QLQQAQAAGAEQEVEK |
| | Cathepsin S sensitive site |
| 1035 | YLEVVLNTLQQASQAQVDK |
| | Cathepsin S sensitive site |
| 1036 | YLEVVLNTLQQASQAQVDK |
| | Cathepsin S sensitive site |
| 1037 | FLSELTQQLAQATGKPPQYI |
| | Cathepsin S sensitive site |
| 1038 | FLSELTQQLAQATGKPPQYIA |
| | Cathepsin S sensitive site |
| 1039 | FLSELTQQLAQATGKPPQYIAVH |
| | Cathepsin S sensitive site |
| 1040 | MTSMGQATWSDPHK |
| | Cathepsin S sensitive site |
| 1041 | EELGLIEQAYDNPHEALSR |
| | Cathepsin S sensitive site |
| 1042 | SLGTIQQCCDAIDHLCR |
| | Cathepsin S sensitive site |
| 1043 | AAAAAAQQQQQCGGGGATKPAVSGK |
| | Cathepsin S sensitive site |
| 1044 | NSCNQCNEPRPEDSR |
| | Cathepsin S sensitive site |
| 1045 | VLIAFAQYLQQCPFEDHVK |
| | Cathepsin S sensitive site |
| 1046 | DSLLQDGEFSMDLR |
| | Cathepsin S sensitive site |
| 1047 | YFLGSIVNFSQDPDVHFK |
| | Cathepsin S sensitive site |
| 1048 | VFSWLQQEGHLSEEEMAR |
| | Cathepsin S sensitive site |
| 1049 | VMSQEIQEQLHK |
| | Cathepsin S sensitive site |
| 1050 | KQEPVKPEEGR |
| | Cathepsin S sensitive site |
| 1051 | LWYCDLQQESSGIAGILK |
| | Cathepsin S sensitive site |
| 1052 | KQEYDESGPSIVHR |
| | Cathepsin S sensitive site |
| 1053 | ETEAICFFVQQFTDMEHNR |
| | Cathepsin S sensitive site |
| 1054 | VTEQGAELSNEER |
| | Cathepsin S sensitive site |
| 1055 | AYMGNVLQGGEGQAPTR |
| | Cathepsin S sensitive site |
| 1056 | AVTEQGHELSNEER |
| | Cathepsin S sensitive site |
| 1057 | VAHTFVVDVAQGTQVTGR |
| | Cathepsin S sensitive site |
| 1058 | VGQGYPHDPPK |
| | Cathepsin S sensitive site |
| 1059 | IYAVEASTMAQHAEVLVK |
| | Cathepsin S sensitive site |
| 1060 | TLAIYFEVVNQHNAPIPQGGR |
| | Cathepsin S sensitive site |
| 1061 | ELAQIAGRPTEDEDEKEK |
| | Cathepsin S sensitive site |
| 1062 | MDEMATTQISKDELDELK |
| | Cathepsin S sensitive site |
| 1063 | YPHLGQKPGGSDFLR |
| | Cathepsin S sensitive site |
| 1064 | TMLELLNQLDGFQPNTQVK |
| | Cathepsin S sensitive site |
| 1065 | ILLELLNQMDGFDQNVNVK |
| | Cathepsin S sensitive site |
| 1066 | LLNQMDGFDTLHR |
| | Cathepsin S sensitive site |
| 1067 | FQESAEAILGQNAAYLGELK |
| | Cathepsin S sensitive site |
| 1068 | HPCFIIAEIGQNHQGDLDVAK |
| | Cathepsin S sensitive site |
| 1069 | LLQDHPWLLSQNLVVKPDQLIK |
| | Cathepsin S sensitive site |
| 1070 | ALPAVQQNNLDEDLIRK |
| | Cathepsin S sensitive site |
| 1071 | ALGQNPTNAEVLK |
| | Cathepsin S sensitive site |
| 1072 | NYQQNYQNSESGEK |
| | Cathepsin S sensitive site |
| 1073 | NYQQNYQNSESGEKNEGSESAPEGQAQQR |
| | Cathepsin S sensitive site |
| 1074 | CGAPSATQPATAETQHIADQVR |
| | Cathepsin S sensitive site |
| 1075 | QAAAAAAQQQQQCGGGGATKPAVSGK |
| | Cathepsin S sensitive site |
| 1076 | IDVTDFLSMTQQDSHAPLR |
| | Cathepsin S sensitive site |
| 1077 | IGSCTQQDVELHVQK |
| | Cathepsin S sensitive site |
| 1078 | LFPLNQQDVPDKFK |
| | Cathepsin S sensitive site |
| 1079 | IGQQPQQPGAPPQQDYTK |
| | Cathepsin S sensitive site |
| 1080 | HQAAAAAAQQQQQCGGGGATKPAVSGK |
| | Cathepsin S sensitive site |
| 1081 | MFTQQQPQELAR |
| | Cathepsin S sensitive site |
| 1082 | LQQQQRPEDAEDGAEGGGK |
| | Cathepsin S sensitive site |
| 1083 | LQQQQRPEDAEDGAEGGGKR |
| | Cathepsin S sensitive site |
| 1084 | SSEADMECLNQRPPENPDTDK |
| | Cathepsin S sensitive site |
| 1085 | SSEADMECLNQRPPENPDTDKNVQ |
| | Cathepsin S sensitive site |
| 1086 | NVNPESQLIQQSEQSESETAGSTK |
| | Cathepsin S sensitive site |
| 1087 | PDNFXFGQSGAGNNWAK |
| | Cathepsin S sensitive site |
| 1088 | SQTCEFNMIEQSGPPHEPR |
| | Cathepsin S sensitive site |
| 1089 | SAVLPPEDMSQSGPSGSHPQGPR |
| | Cathepsin S sensitive site |
| 1090 | IEFLQSHENQEIYQK |
| | Cathepsin S sensitive site |
| 1091 | NTVSQSISGDPEIDKK |
| | Cathepsin S sensitive site |
| 1092 | LLIHQSLAGGIIGVK |
| | Cathepsin S sensitive site |
| 1093 | MVXYLANLTQSQIALNEK |
| | Cathepsin S sensitive site |
| 1094 | PPKPEPFQFGQSSQKPPVAGGK |
| | Cathepsin S sensitive site |
| 1095 | NGNYCVLQMDQSYKPDENEVR |
| | Cathepsin S sensitive site |
| 1096 | ILVGDVGQTVDDPYATFVK |
| | Cathepsin S sensitive site |
| 1097 | ADDVDLEQVANETHGHVG |
| | Cathepsin S sensitive site |
| 1098 | ADDVDLEQVANETHGHVGA |
| | Cathepsin S sensitive site |
| 1099 | SINFLHQVCHDQTPTTK |
| | Cathepsin S sensitive site |
| 1100 | CTTVAFTQVNSEDKGALAK |
| | Cathepsin S sensitive site |
| 1101 | QQLQQVPGLLHR |
| | Cathepsin S sensitive site |
| 1102 | SQQYPAARPAEP |
| | Cathepsin S sensitive site |
| 1103 | DFCIQVGRNIIHGSDSVK |
| | Cathepsin S sensitive site |
| 1104 | VLMSHLGRPDGVPMPDKY |
| | Cathepsin S sensitive site |
| 1105 | VLMSHLGRPDGVPMPDKYS |
| | Cathepsin S sensitive site |
| 1106 | AQVARPGGDTIFGK |
| | Cathepsin S sensitive site |
| 1107 | FMSVQRPGPYDRPGTAR |
| | Cathepsin S sensitive site |
| 1108 | VLVERSAAETVTK |
| | Cathepsin S sensitive site |
| 1109 | FLPSARSSPASSPE |
| | Cathepsin S sensitive site |
| 1110 | RPELGSEGLGSAAHGSQPDLR |
| | Cathepsin S sensitive site |
| 1111 | MPDQGMTSADDFFQGTK |
| | Cathepsin S sensitive site |
| 1112 | DVPAPSTSADKVESLDVDSEAK |
| | Cathepsin S sensitive site |
| 1113 | QVCLPVISAENWKPATK |
| | Cathepsin S sensitive site |
| 1114 | GFGSGDDPYSSAEPHVSGVK |
| | Cathepsin S sensitive site |
| 1115 | EFGDNTAGCTSAGPHFNPLSR |
| | Cathepsin S sensitive site |
| 1116 | TYFSCTSAHTSTGDGTAMITR |
| | Cathepsin S sensitive site |
| 1117 | TYSLGSALRPSTSR |
| | Cathepsin S sensitive site |
| 1118 | VSDQELQSANASVDDSR |
| | Cathepsin S sensitive site |
| 1119 | APGSAAPAAGSAPAAAEEK |
| | Cathepsin S sensitive site |
| 1120 | APGSAAPAAGSAPAAAEEK |
| | Cathepsin S sensitive site |
| 1121 | APGSAAPAAGSAPAAAEEK |
| | Cathepsin S sensitive site |
| 1122 | APGSAAPAAGSAPAAAEEKK |
| | Cathepsin S sensitive site |
| 1123 | NEGSESAPEGQAQQR |
| | Cathepsin S sensitive site |
| 1124 | QVEPLDPPAGSAPGEHVFVK |
| | Cathepsin S sensitive site |
| 1125 | PTGEAGPSCSSASDKLPR |
| | Cathepsin S sensitive site |
| 1126 | YYTSASGDEMVSLK |
| | Cathepsin S sensitive site |
| 1127 | NQQGAHSALSSASTSSHNLQ |
| | Cathepsin S sensitive site |
| 1128 | EALLSSAVDHGSDEVK |
| | Cathepsin S sensitive site |
| 1129 | DYMVEIDILASCDHPNIVK |
| | Cathepsin S sensitive site |
| 1130 | MESCGIHETTF |
| | Cathepsin S sensitive site |
| 1131 | QLSSCLPNIVPK |
| | Cathepsin S sensitive site |
| 1132 | LIXSDGHEFIVK |
| | Cathepsin S sensitive site |
| 1133 | EIVDGGVILESDPQQVVHR |
| | Cathepsin S sensitive site |
| 1134 | SLEDALSSDTSGHFR |
| | Cathepsin S sensitive site |
| 1135 | VGVEAHVDIHSDVPKGANSF |
| | Cathepsin S sensitive site |
| 1136 | VILGSEAAQQHPEEVR |
| | Cathepsin S sensitive site |
| 1137 | XSEDKGALAK |
| | Cathepsin S sensitive site |
| 1138 | GGTSXXSSEGTQHSYSEEEK |
| | Cathepsin S sensitive site |
| 1139 | CALGGTSELSSEGTQHSYSEEEKY |
| | Cathepsin S sensitive site |
| 1140 | MDPNIVGSEHYDVAR |
| | Cathepsin S sensitive site |
| 1141 | SPAPSSVPLGSEKPSNVSQDR |
| | Cathepsin S sensitive site |
| 1142 | MTQAGVEELESENKIPATQK |
| | Cathepsin S sensitive site |
| 1143 | MLLDSEQHPCQLK |
| | Cathepsin S sensitive site |
| 1144 | GLGNVLGGLISGAGGGGGGGGGGGGGGGGGGGGTAMR |
| | Cathepsin S sensitive site |
| 1145 | IMDDLTEVLCSGAGGVHSGGSGDGAGSGGPGAQNHVK |
| | Cathepsin S sensitive site |
| 1146 | ATQGAAAAAGSGAGTGGGTASGGTEGGSAESEGAK |
| | Cathepsin S sensitive site |
| 1147 | LEPAPLDSLCSGASAEEPTSHR |
| | Cathepsin S sensitive site |
| 1148 | VIGSGCNLDSAR |
| | Cathepsin S sensitive site |
| 1149 | WXLNSGDGAFYGPK |
| | Cathepsin S sensitive site |
| 1150 | FFDMAYQGFASGDGDKDAWAVR |
| | Cathepsin S sensitive site |
| 1151 | VSIEDSVISLSGDHCIIGR |
| | Cathepsin S sensitive site |
| 1152 | EYLLSGDISEAEHCLK |
| | Cathepsin S sensitive site |
| 1153 | DDGLFSGDPNWFPK |
| | Cathepsin S sensitive site |
| 1154 | WQHDLFDSGFGGGAGVETGGK |
| | Cathepsin S sensitive site |
| 1155 | DSVWGSGGGQQSVNHLVK |
| | Cathepsin S sensitive site |
| 1156 | PEGPNEAEVTSGKPEQEVPDAEEEK |
| | Cathepsin S sensitive site |
| 1157 | VQSGNINAAK |
| | Cathepsin S sensitive site |
| 1158 | YQYGGLNSGRPVTPPR |
| | Cathepsin S sensitive site |
| 1159 | VLQATVVAVGSGSKGKGGEIQPVSVK |
| | Cathepsin S sensitive site |
| 1160 | GILFVGSGVSGGEEGAR |
| | Cathepsin S sensitive site |
| 1161 | IEFLQSHENQEIYQK |
| | Cathepsin S sensitive site |
| 1162 | LDEVITSHGAIEPDKDNVR |
| | Cathepsin S sensitive site |
| 1163 | EHPVIESHPDNALEDLR |
| | Cathepsin S sensitive site |
| 1164 | LIQSHPESAEDLQEK |
| | Cathepsin S sensitive site |
| 1165 | TIVITSHPGQIVK |
| | Cathepsin S sensitive site |
| 1166 | IEWLESHQDADIEDFK |
| | Cathepsin S sensitive site |
| 1167 | GYPHLCSICDLPVHSNK |
| | Cathepsin S sensitive site |
| 1168 | SEPCALCSLHSIGKIGGAQNR |
| | Cathepsin S sensitive site |
| 1169 | LQSIGTENTEENR |
| | Cathepsin S sensitive site |
| 1170 | LFIHESIHDEVVNR |
| | Cathepsin S sensitive site |
| 1171 | VTFNINNSIPPTFDGEEEPSQGQK |
| | Cathepsin S sensitive site |
| 1172 | NLNTLCWAIGSISGAMHEEDEKR |
| | Cathepsin S sensitive site |
| 1173 | EASATNSPCTSKPATPAPSEK |
| | Cathepsin S sensitive site |
| 1174 | PPNPNCYVCASKPEVTVR |
| | Cathepsin S sensitive site |
| 1175 | ICSKPVVLPK |
| | Cathepsin S sensitive site |
| 1176 | QFHFHWGSLDGQGSEHTVDK |
| | Cathepsin S sensitive site |
| 1177 | QFHFHWGSLDGQGSEHTVDKK |
| | Cathepsin S sensitive site |
| 1178 | GNPICSLHDQGAGGNGNVLK |
| | Cathepsin S sensitive site |
| 1179 | EANFTVSSMHGDMPQK |
| | Cathepsin S sensitive site |
| 1180 | NQLTSNPENTVFDAK |
| | Cathepsin S sensitive site |
| 1181 | QVLVGSYCVFSNQGGLVHPK |
| | Cathepsin S sensitive site |
| 1182 | DLQSNVEHLTEK |
| | Cathepsin S sensitive site |
| 1183 | EEMQSNVEVVHTYR |
| | Cathepsin S sensitive site |
| 1184 | APVQPQQSPAAAPGGTDEKPSGK |
| | Cathepsin S sensitive site |
| 1185 | APVQPQQSPAAAPGGTDEKPSGK |
| | Cathepsin S sensitive site |
| 1186 | NDGPVTIELESPAPGTATSDPK |
| | Cathepsin S sensitive site |
| 1187 | INSLFLTDLYSPEYPGPSHR |
| | Cathepsin S sensitive site |
| 1188 | NGSLDSPGKQDTEEDEEEDEKDK |
| | Cathepsin S sensitive site |
| 1189 | SAAAASAASGSPGPGEGSAGGEKR |
| | Cathepsin S sensitive site |
| 1190 | SAAAASAASGSPGPGEGSAGGEKR |
| | Cathepsin S sensitive site |
| 1191 | NADTDLVSWLSPHDPNSVVTK |
| | Cathepsin S sensitive site |
| 1192 | LSPPYSSPQEFAQDVGR |
| | Cathepsin S sensitive site |
| 1193 | IIAFVGSPVEDNEKDLVK |
| | Cathepsin S sensitive site |
| 1194 | MESQEPTESSQNGK |
| | Cathepsin S sensitive site |
| 1195 | AXASQLDCNFLK |
| | Cathepsin S sensitive site |
| 1196 | SQGDSISSQLGPIHPPPR |
| | Cathepsin S sensitive site |
| 1197 | LGGLLKPTVASQNQNLPVAK |
| | Cathepsin S sensitive site |
| 1198 | SSWGMMGMLASQQNQSGPSGNNQNQGNMQR |
| | Cathepsin S sensitive site |
| 1199 | DEYLINSQTTEHIVK |
| | Cathepsin S sensitive site |
| 1200 | YQLGLAYGYNSQYDEAVAQFSK |
| | Cathepsin S sensitive site |
| 1201 | GLLLLSVVVTSRPEAFQPH |
| | Cathepsin S sensitive site |
| 1202 | RPASVSSSAAVEHEQR |
| | Cathepsin S sensitive site |
| 1203 | FGIVTSSAGTGTTEDTEAK |
| | Cathepsin S sensitive site |
| 1204 | FGIVTSSAGTGTTEDTEAKK |
| | Cathepsin S sensitive site |
| 1205 | STASAPAAVNSSASADKPLSNMK |
| | Cathepsin S sensitive site |
| 1206 | EALLSSAVDHGSDEVK |
| | Cathepsin S sensitive site |
| 1207 | VSWLEYESSFSNEEAQK |
| | Cathepsin S sensitive site |
| 1208 | IXXGSSGAQGSGGGSTSAHY |
| | Cathepsin S sensitive site |
| 1209 | HIGGPPGFASSSGKPGPTVIK |
| | Cathepsin S sensitive site |
| 1210 | FEMYEPSELESSHLTDQDNEIR |
| | Cathepsin S sensitive site |
| 1211 | SPDDDLGSSNWEAADLGNEER |
| | Cathepsin S sensitive site |
| 1212 | GDSQVSSNPTSSPPGEAPAPVSVDSEPS |
| | Cathepsin S sensitive site |
| 1213 | FVNGQPRPLESSQVKYLR |
| | Cathepsin S sensitive site |
| 1214 | KPLTSSSAAPQRPISTQR |
| | Cathepsin S sensitive site |
| 1215 | IHIGGPPGFASSSGKPGPTVIK |
| | Cathepsin S sensitive site |
| 1216 | ELVSSSSSGSDSDSEVDKK |
| | Cathepsin S sensitive site |
| 1217 | LLDSSTVTHLFK |
| | Cathepsin S sensitive site |
| 1218 | PPPAAPPPSSSSVPEAGGPPIKK |
| | Cathepsin S sensitive site |
| 1219 | YVELFLNSTAGASGGAYEHR |
| | Cathepsin S sensitive site |
| 1220 | SHELSDFGLESTAGEIPVVAIR |
| | Cathepsin S sensitive site |
| 1221 | ECEEEAINIQSTAPEEEHESPR |
| | Cathepsin S sensitive site |
| 1222 | EGTGSTATSSSSTAGAAGK |
| | Cathepsin S sensitive site |
| 1223 | PLHSIISSTESVQGSTSK |
| | Cathepsin S sensitive site |
| 1224 | VAFTGSTEVGHLIQK |
| | Cathepsin S sensitive site |
| 1225 | LALVTGGEIASTFDHPELVK |
| | Cathepsin S sensitive site |
| 1226 | ATIELCSTHANDASALR |
| | Cathepsin S sensitive site |
| 1227 | VHITLSTHECAGLSER |
| | Cathepsin S sensitive site |
| 1228 | EEEEPQAPQESTPAPPKK |
| | Cathepsin S sensitive site |
| 1229 | SITILSTPEGTSAACK |
| | Cathepsin S sensitive site |
| 1230 | ETLASSDSFASTQPTHSWK |
| | Cathepsin S sensitive site |
| 1231 | VVVLMGSTSDLGHCEK |
| | Cathepsin S sensitive site |
| 1232 | VLLSNLSSTSHVPEVDPGSAELQK |
| | Cathepsin S sensitive site |
| 1233 | LFDSTTLEHQK |
| | Cathepsin S sensitive site |
| 1234 | TQLEGLQSTVTGHVER |
| | Cathepsin S sensitive site |
| 1235 | GSESGGSAVDSVAGEHSVSGR |
| | Cathepsin S sensitive site |
| 1236 | YEILQSVDDAAIVIK |
| | Cathepsin S sensitive site |
| 1237 | NDLSICGTLHSVDQYLNIK |
| | Cathepsin S sensitive site |
| 1238 | ILDSVGIEADDDR |
| | Cathepsin S sensitive site |
| 1239 | ILDSVGIEADDDRLNK |
| | Cathepsin S sensitive site |
| 1240 | IYVASVHQDLSDDDIK |
| | Cathepsin S sensitive site |
| 1241 | ELQSVKPQEAPK |
| | Cathepsin S sensitive site |
| 1242 | HYTEGAELVDSVLDVVRK |
| | Cathepsin S sensitive site |
| 1243 | LAEGSVTSVGSVNPAENFR |
| | Cathepsin S sensitive site |
| 1244 | GSPTSLGTWGSWIGPDHDK |
| | Cathepsin S sensitive site |
| 1245 | VLNSYWVGEDSTYK |
| | Cathepsin S sensitive site |
| 1246 | SLGTADVHFER |
| | Cathepsin S sensitive site |
| 1247 | MAGTAFDFENMK |
| | Cathepsin S sensitive site |
| 1248 | VLATAFDTTLGGR |
| | Cathepsin S sensitive site |
| 1249 | VELFLNSTAGASGGAYEHR |
| | Cathepsin S sensitive site |
| 1250 | APPPSGSAVSTAPQQKPIGK |
| | Cathepsin S sensitive site |
| 1251 | SQIFSTASDNQPTVTIK |
| | Cathepsin S sensitive site |
| 1252 | IYWGTATTGKPHVA |
| | Cathepsin S sensitive site |
| 1253 | MMLGTEGGEGFVVK |
| | Cathepsin S sensitive site |
| 1254 | FGAVWTGDNTAEWDHLK |
| | Cathepsin S sensitive site |
| 1255 | VSHVSTGGGASLELL |
| | Cathepsin S sensitive site |
| 1256 | VSHVSTGGGASLELLE |
| | Cathepsin S sensitive site |
| 1257 | VSHVSTGGGASLELLEGK |
| | Cathepsin S sensitive site |
| 1258 | ILISLATGHREEGGENLDQAR |
| | Cathepsin S sensitive site |
| 1259 | TLDQCIQTGVDNPGHPFIK |
| | Cathepsin S sensitive site |
| 1260 | SGFTLDDVIQTGVDNPGHPY |
| | Cathepsin S sensitive site |
| 1261 | DLTTGYDDSQPDKK |
| | Cathepsin S sensitive site |
| 1262 | FFFGTHETAFLGPK |
| | Cathepsin S sensitive site |
| 1263 | FPSLLTHNENMVAK |
| | Cathepsin S sensitive site |
| 1264 | YEDICPSTHNMDVPNIK |
| | Cathepsin S sensitive site |
| 1265 | DYALHWLVLGTHTSDEQNHLVVAR |
| | Cathepsin S sensitive site |
| 1266 | FGTINIVHPK |
| | Cathepsin S sensitive site |
| 1267 | SMVNTKPEKTEEDSEEVR |
| | Cathepsin S sensitive site |
| 1268 | VTLLTPAGATGSGGGTSGDSSKGEDKQDR |
| | Cathepsin S sensitive site |
| 1269 | PGETLTEILETPATSEQEAEHQR |
| | Cathepsin S sensitive site |
| 1270 | NSVQTPVENSTNSQHQVK |
| | Cathepsin S sensitive site |
| 1271 | AXXITPVPGGVGPMTV |
| | Cathepsin S sensitive site |
| 1272 | STVLTPMFVETQASQGTLQTR |
| | Cathepsin S sensitive site |
| 1273 | TFTTQETITNAETAK |
| | Cathepsin S sensitive site |
| 1274 | SPVSTRPLPSASQK |
| | Cathepsin S sensitive site |
| 1275 | TNEQWQMSLGTSEDHQHFT |
| | Cathepsin S sensitive site |
| 1276 | QEIIXQLDVTTSEYEKEK |
| | Cathepsin S sensitive site |
| 1277 | LLAFLLAELGTSGSIDGNNQLVIK |
| | Cathepsin S sensitive site |
| 1278 | LXNMEIGTSLFDEEGAK |
| | Cathepsin S sensitive site |
| 1279 | AEKPAETPVATSPTATDSTSGDSSR |
| | Cathepsin S sensitive site |
| 1280 | LLETTDRPDGHQNNLR |
| | Cathepsin S sensitive site |
| 1281 | AQTITSEXXSTTTTTHITK |
| | Cathepsin S sensitive site |
| 1282 | ADAVGMSTVPEVIVAR |
| | Cathepsin S sensitive site |
| 1283 | IHFPLATYAPVISAEK |
| | Cathepsin S sensitive site |
| 1284 | DTXVXXDTYNCDLHFK |
| | Cathepsin S sensitive site |
| 1285 | VVIGMDVAASEFFR |
| | Cathepsin S sensitive site |
| 1286 | GXXXXXIGLXVADLAESIMK |
| | Cathepsin S sensitive site |
| 1287 | ANPQVGVAFPHIK |
| | Cathepsin S sensitive site |
| 1288 | PQEAKPQEAAVAPEKPPASDETK |
| | Cathepsin S sensitive site |
| 1289 | HFSVEGQLEFR |
| | Cathepsin S sensitive site |
| 1290 | VATLGVEVHPLVFH |
| | Cathepsin S sensitive site |
| 1291 | HWPFQVINDGDKPK |
| | Cathepsin S sensitive site |
| 1292 | LPVPAFNVINGGSHAGNK |
| | Cathepsin S sensitive site |
| 1293 | EVANGIESLGVKPDLPPPPSK |
| | Cathepsin S sensitive site |
| 1294 | TYYDVLGVKPNATQEELKK |
| | Cathepsin S sensitive site |
| 1295 | ETVAVKPTENNEEEFTSK |
| | Cathepsin S sensitive site |
| 1296 | SLLVNPEGPTLMR |
| | Cathepsin S sensitive site |
| 1297 | NWMNSLGVNPHVNHLY |
| | Cathepsin S sensitive site |
| 1298 | HGLLVPNNTTDQELQHIR |
| | Cathepsin S sensitive site |
| 1299 | QELEFLEVQEEYIKDEQK |
| | Cathepsin S sensitive site |
| 1300 | LEGTLLKPNMVTPGHACTQK |
| | Cathepsin S sensitive site |
| 1301 | FVNVVPTFGKK |
| | Cathepsin S sensitive site |
| 1302 | EDLVFIFWAPESAPLK |
| | Cathepsin S sensitive site |
| 1303 | AIYIDASCLTWEGQQFQGK |
| | Cathepsin S sensitive site |
| 1304 | EQPQHPLHVTYAGAAVDELGK |
| | Cathepsin S sensitive site |
| 1305 | SPDGHLFQVEYAQEAVKK |
| | Cathepsin S sensitive site |
| 1306 | NYKPPAQK |
| | Cathepsin S sensitive site |
| 1307 | VYNYNHLMPTR |
| | Cathepsin S sensitive site |
| 1308 | LAEAELEYNPEHVSR |
| | Cathepsin S sensitive site |
| 1309 | MPYQYPALTPEQK |
| | Cathepsin S sensitive site |
| 1310 | TSSANNPNLMYQDECDRR |
| | Cathepsin S sensitive site |
| 1311 | VGINYQPPTVVPGGDLAK |
| | Cathepsin S sensitive site |
| 1312 | YMACCXLYRGDVVPK |
| | Cathepsin S sensitive site |
| 1313 | SYCYVSKEELK |
| | Cathepsin S sensitive site |
| 1314 | AAGCTTAGCGGCCGCACCATGCGGGTCACGGCGCCCCGAACC |
| | (sec sense pri mer) |
| 1315 | CTGCAGGGAGCCGGCCCAGGTCTCGGTCAG |
| | (sec antisense pri mer) |
| 1316 | GGATCCATCGTGGGCATTGTTGCTGGCCTGGCT |
| | (MITD sense pri mer) |
| 1317 | GAATTCAGTCTCGAGTCAAGCTGTGAGAGACACATCAGAGCC |
| | (MITD antisense pri mer) |
| 1318 | MQIFVKTLTGKTITLEVEPSDTIENVKAKIQDKEGIPPDQQRLIFAGKQLEDGRTLSDYNIQKESTLHLVLRLRGG |
| | (ubiquitin) |
| 1319 | 5′^{7Me}GₚₚₚG_{2′OMe}GGAAAUAAGAGAGAAAAGAAGAGUAAGAAGAAAUAUAAGAGCCACCAUGCCCCACAGUA |
| | GCCUCCACCCCAGCAUCCCCUGCCCCAGAGGCCACGGCGCACAGAAGGCCGCCCUGGUGCUGCUGAGCGCC |
| | UGUCUGGUGACCCUGUGGGGUCUGGGCGAGCCCCCCGAGCACACCCUGCGGUACCUCGUGCUGCAUCUG |
| | GCCAGCCUGCAGCUGGGCCUGCUGCUGAACGGCGUGUGCAGCCUGGCCGAAGAGCUGAGACACAUCCACA |
| | GCAGAUACAGAGGCUCCUACUGGAGAACCGUCAGAGCCUGCCUCGGCUGUCCCCUGAGAAGAGGCGCCCU |
| | GCUGCUCCUGAGCAUCUACUUCUACUACAGCCUGCCCAACGCCGUGGGCCCCCCCUUCACCUGGAUGCUG |
| | GCCCUGCUGGGCCUGAGCCAGGCCCUGAACAUCCUGCUGGGCCUGAAGGGCUUGGCCCCCGCCGAGAUCU |
| | CCGCCGUGUGCGAGAAGGGCAACUUCAACAUGGCCCAUGGCCUUGCCUGGUCCUACUACAUCGGCUACCU |
| | GAGACUGAUCCUGCCCGAGCUGCAGGCCAGAAUCAGAACCUACAACCAGCACUACAACAACCUGCUGAGAG |
| | GCGCCGUGAGCCAAAGACUGUACAUCCUGCUGCCCCUGGACUGCGGCGUGCCCGACAACCUUAGCAUGGC |
| | CGACCCCAACAUCAGAUUCCUGGACAAGCUGCCCCAGCAGACCGGCGACCACGCCGGCAUCAAGGACAGAG |
| | UGUACAGCAACAGCAUCUACGAGCUGCUGGAGAACGGCCAGAGAGCCGGCACCUGCGUGCUGGAGUACGC |
| | CACCCCCCUGCAGACCCUGUUCGCCAUGAGCCAGUACAGCCAGGCCGGCUUCAGCAGAGAGGACAGACUG |
| | GAGCAAGCCAAGCUGUUCUGCAGAACCCUGGAGGACAUCCUGGCGGACGCCCCCGAGAGCCAAAACAACU |
| | GCAGACUGAUCGCCUACCAGGAGCCCGCCGACGACAGCAGCUUCAGCCUGAGCCAGGAAGUGCUGAGACA |
| | CCUGAGACAGGAAGAGAAGGAGGAGGUGACCGUGGGAAGCCUGAAGACCAGCGCCGUGCCCAGCACCAGC |
| | ACCAUGAGCCAGGAGCCCGAGCUGCUGAUCAGCGGCAUGGAGAAGCCCCUGCCCCUGAGAACCGACUUCA |
| | GCUGAUAAUAGGCUGGAGCCUCGGUGGCCUAGCUUCUUGCCCCUUGGGCCUCCCCCCAGCCCCUCCUCCC |
| | CUUCCUGCACCCGUACCCCCCAAACACCAUUGUCACACUCCAGUGGUCUUUGAAUAAAGUCUGAGUGGGC |
| | GGCAAAAAAAAAAAAAAAAAAAAAAAAAAAAAAAAAAAAAAAAAAAAAAAAAAAAAAAAAAAAAAAAAAAA |
| | AAAAAAAAAAAAAAAAAAAAAAAAAAAAAAAAUCUAG_{OH}3′ |
| | Where: A,C G & U = AMP, CMP, GMP & N1-ΨUMP, respectively; Me = methyl; p = inorganic |
| | phosphate; underline = miR-122 binding site |
| | (STING mRNA sequence; CX-012871) |
| 1320 | AUGCCCCACAGUAGCCUCCACCCCAGCAUCCCCUGCCCCAGAGGCCACGGCGCACAGAAGGCCGCCCUGGU |
| | GCUGCUGAGCGCCUGUCUGGUGACCCUGUGGGGUCUGGGCGAGCCCCCCGAGCACACCCUGCGGUACCUC |
| | GUGCUGCAUCUGGCCAGCCUGCAGCUGGGCCUGCUGCUGAACGGCGUGUGCAGCCUGGCCGAAGAGCUG |
| | AGACACAUCCACAGCAGAUACAGAGGCUCCUACUGGAGAACCGUCAGAGCCUGCCUCGGCUGUCCCCUGA |
| | GAAGAGGCGCCCUGCUGCUCCUGAGCAUCUACUUCUACUACAGCCUGCCCAACGCCGUGGGCCCCCCCUUC |
| | ACCUGGAUGCUGGCCCUGCUGGGCCUGAGCCAGGCCCUGAACAUCCUGCUGGGCCUGAAGGGCUUGGCC |
| | CCCGCCGAGAUCUCCGCCGUGUGCGAGAAGGGCAACUUCAACAUGGCCCAUGGCCUUGCCUGGUCCUACU |
| | ACAUCGGCUACCUGAGACUGAUCCUGCCCGAGCUGCAGGCCAGAAUCAGAACCUACAACCAGCACUACAAC |
| | AACCUGCUGAGAGGCGCCGUGAGCCAAAGACUGUACAUCCUGCUGCCCCUGGACUGCGGCGUGCCCGACA |
| | ACCUUAGCAUGGCCGACCCCAACAUCAGAUUCCUGGACAAGCUGCCCCAGCAGACCGGCGACCACGCCGGC |
| | AUCAAGGACAGAGUGUACAGCAACAGCAUCUACGAGCUGCUGGAGAACGGCCAGAGAGCCGGCACCUGCG |
| | UGCUGGAGUACGCCACCCCCCUGCAGACCCUGUUCGCCAUGAGCCAGUACAGCCAGGCCGGCUUCAGCAG |
| | AGAGGACAGACUGGAGCAAGCCAAGCUGUUCUGCAGAACCCUGGAGGACAUCCUGGCGGACGCCCCCGAG |
| | AGCCAAAACAACUGCAGACUGAUCGCCUACCAGGAGCCCGCCGACGACAGCAGCUUCAGCCUGAGCCAGGA |
| | AGUGCUGAGACACCUGAGACAGGAAGAGAAGGAGGAGGUGACCGUGGGAAGCCUGAAGACCAGCGCCGU |
| | GCCCAGCACCAGCACCAUGAGCCAGGAGCCCGAGCUGCUGAUCAGCGGCAUGGAGAAGCCCCUGCCCCUGA |
| | GAACCGACUUCAGC |
| | (huSTING(V155M); no epitope tag; nucleotide sequence) |
| 1321 | 5′^{7Me}GₚₚₚG_{2′OMe}GGAAAUAAGAGAGAAAAGAAGAGUAAGAAGAAAUAUAAGAGCCACCAUGACCGAGUACA |
| | AGCUCGUGGUCGUCGGCGCCGACGGGGUAGGCAAGUCCGCUCUGACCAUUCAGCUCAUCCAGAUGACGG |
| | AGUACAAACUCGUGGUAGUGGGAGCCGUGGGUGUGGGCAAGAGCGCGCUCACCAUCCAACUCAUCCAAA |
| | UGACCGAAUAUAAACUCGUCGUGGUGGGAGCCGGCGACGUGGGAAAGAGCGCCCUUACCAUCCAGUUAA |
| | UCCAGAUGACAGAAUACAAGCUGGUGGUGGUCGGUGCCUGCGGCGUGGGUAAGUCCGCCCUGACAAUCC |
| | AGCUGAUCCAGUGAUAAUAGGCUGGAGCCUCGGUGGCCAUGCUUCUUGCCCCUUGGGCCUCCCCCCAGCC |
| | CCUCCUCCCCUUCCUGCACCCGUACCCCCGUGGUCUUUGAAUAAAGUCUGAGUGGGCGGCAAAAAAAAAA |
| | AAAAAAAAAAAAAAAAAAAAAAAAAAAAAAAAAAAAAAAAAAAAAAAAAAAAAAAAAAAAAAAAAAAAAAA |
| | AAAAAAAAAAAAAAAAAAAUCUAG_{OH}3′ |
| | Where: A,C G & U = AMP, CMP, GMP & N1-ΨUMP, respectively; Me = methyl; p = inorganic |
| | phosphate |
| | (KRAS concate mer mRNA sequence; CX-012908) |
| 1322 | AUGACCGAGUACAAGCUCGUGGUCGUCGGCGCCGACGGGGUAGGCAAGUCCGCUCUGACCAUUCAGCUC |
| | AUCCAGAUGACGGAGUACAAACUCGUGGUAGUGGGAGCCGUGGGUGUGGGCAAGAGCGCGCUCACCAUC |
| | CAACUCAUCCAAAUGACCGAAUAUAAACUCGUCGUGGUGGGAGCCGGCGACGUGGGAAAGAGCGCCCUU |
| | ACCAUCCAGUUAAUCCAGAUGACAGAAUACAAGCUGGUGGUGGUCGGUGCCUGCGGCGUGGGUAAGUCC |
| | GCCCUGACAAUCCAGCUGAUCCAG |
| | (KRAS(G12D G12V G13D G12C) 100 mer “4MUT” nt. seq) |
| 1323 | GGAAAUAAGAGAGAAAAGAAGAGUAAGAAGAAAUAUAAGAGCCACC |
| | (5′ UTR) |
| 1324 | CCUUAGCAGAGCUGUGGAGUGUGACAAUGGUGUUUGUGUCUAAACUAUCAAACGCCAUUAUCACACUAA |
| | AUAGCUACUGCUAGGC |
| | (miR-122) |
| 1325 | UAUUUAGUGUGAUAAUGGCGUU |
| | (miR-122-3p binding site) |
| 1326 | CAAACACCAUUGUCACACUCCA |
| | (miR-122-5p binding site) |
| 1327 | MENLKHIITLGQVIHKRCEEMKYCKKQCRRLGHRVLGLIKPLEMLQDQGKRSVPSEKLTTAMNRFKAALEEANGEIE |
| | KFSNRSNICRFLTASQDKILFKDVNRKLSDVWKELSLLLQVEQRMPVSPISQGASWAQEDQQDADEDRRAFQMLR |
| | RDNEKIEASLRRLEINMKEIKETLRQY |
| | (human MLKL(1-180) ORF amino acid sequence; no epitope tag) |
| 1328 | MDKLGQIIKLGQLIYEQCEKMKYCRKQCQRLGNRVHGLLQPLQRLQAQGKKNLPDDITAALGRFDEVLKEANQQIE |
| | KFSKKSHIWKFVSVGNDKILFHEVNEKLRDVWEELLLLLQVYHWNTVSDVSQPASWQQEDRQDAEEDGNENMKV |
| | ILMQLQISVEEINKTLKQCSLKPTQEIPQD |
| | (mouse MLKL(1-180) ORF amino acid sequence; no epitope tag) |
| 1329 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNgvqvetispgdgrtf |
| | pkrgqtcvvhytgmledgkkVdssrdrnkpfkfmlgkqevirgweegvaqmsvgqrakltispdyaygatghpgiipphatlvfdv |
| | ellklegvqvetispgdgrtfpkrgqtcvvhytgmledgkkVdssrdrnkpfkfmlgkqevirgweegvaqmsvgqrakltispdy |
| | aygatghpgiipphatlvfdvellkle |
| | (muRIPK3AC-2xFV; TH1001 with no epitope tag) |
| 1330 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNggggsggggsKKRL |
| | AYAIIQFLHDQLRHGGLSSDAQESLEVAIQCLETAFGVTVEDSDLALKKRLAYAIIQFLHDQLRHGGLSSDAQESLEVAI |
| | QCLETAFGVTVEDSDLAL |
| | (muRIPK3AC-2xSGTA.DM; TH1003 with no epitope tag) |
| 1331 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNggggsggggsGA |
| | MEPENKYLPELMAEKDSLDPSFTHAMQLLTAEIEKIQKGGAMEPENKYLPELMAEKDSLDPSFTHAMQLLTAEIEKI |
| | QKG |
| | (muRIPK3AC-2xSrc.DM; TH1005 no epitope tag) |
| 1332 | MSCVKLWPSGAPAPLVSIEELENQELVGKGGFGTVFRAQHRKWGYDVAVKIVNSKAISREVKAMASLDNEFVLRLE |
| | GVIEKVNWDQDPKPALVTKFMENGSLSGLLQSQCPRPWPLLCRLLKEVVLGMFYLHDQNPVLLHRDLKPSNVLLDP |
| | ELHVKLADFGLSTFQGGSQSGTGSGEPGGTLGYLAPELFVNVNRKASTASDVYSFGILMWAVLAGREVELPTEPSLV |
| | YEAVCNRQNRPSLAELPQAGPETPGLEGLKELMQLCWSSEPKDRPSFQECLPKTDEVFQMVENNMNAAVSTVKDF |
| | LSQLRSSNRRFSIPESGQGGTEMDGFRRTIENQHSRNDVMVSEWLNKLNLEEPPSSVPKKCPSLTKRSRAQEEQVP |
| | QAWTAGTSSDSMAQPPQTPETSTFRNQMPSPTSTGTPSPGPRGNQGAERQGMNWSCRTPEPNPVTGRPLVNTF |
| | GVQVETISPGDGRTFPKRGQTCVVHYTGMLEDGKKVDSSRDRNKPFKFMLGKQEVIRGWEEGVAQMSVGQRAKL |
| | TISPDYAYGATGHPGIIPPHATLVFDVELLKLETRGVQVETISPGDGRTFPKRGQTCVVHYTGMLEDGKKVDSSRDRN |
| | KPFKFMLGKQEVIRGWEEGVAQMSVGQRAKLTISPDYAYGATGHPGIIPPHATLVFDVELLKLETS |
| | (huRIPK3.del.C-2xFv; TH1007 with no epitope tag) |
| 1333 | MSCVKLWPSGAPAPLVSIEELENQELVGKGGFGTVFRAQHRKWGYDVAVKIVNSKAISREVKAMASLDNEFVLRLE |
| | GVIEKVNWDQDPKPALVTKFMENGSLSGLLQSQCPRPWPLLCRLLKEVVLGMFYLHDQNPVLLHRDLKPSNVLLDP |
| | ELHVKLADFGLSTFQGGSQSGTGSGEPGGTLGYLAPELFVNVNRKASTASDVYSFGILMWAVLAGREVELPTEPSLV |
| | YEAVCNRQNRPSLAELPQAGPETPGLEGLKELMQLCWSSEPKDRPSFQECLPKTDEVFQMVENNMNAAVSTVKDF |
| | LSQLRSSNRRFSIPESGQGGTEMDGFRRTIENQHSRNDVMVSEWLNKLNLEEPPSSVPKKCPSLTKRSRAQEEQVP |
| | QAWTAGTSSDSMAQPPQTPETSTFRNQMPSPTSTGTPSPGPRGNQGAERQGMNWSCRTPEPNPVTGRPLVNTF |
| | (huRIPK3.del.C; TH1008 with no epitope tag) |
| 1334 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVENN |
| | (muRIPK3.del.C; TH1009 with no epitope tag) |
| 1335 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNCSEVQIGNYNSL |
| | VAPPRTTASSSAKYDQAQFGRGRGWQPFHKggggsggggsKKRLAYAIIQFLHDQLRHGGLSSDAQESLEVAIQCLET |
| | AFGVTVEDSDLALKKRLAYAIIQFLHDQLRHGGLSSDAQESLEVAIQCLETAFGVTVEDSDLAL |
| | (muRIPK3-2xSGTA.DM; TH1010 with no epitope tag) |
| 1336 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNCSEVQIGNYNSL |
| | VAPPRTTASSSAKYDQAQFGRGRGWQPFHKggggsggggsGAMEPENKYLPELMAEKDSLDPSFTHAMQLLTAEIEK |
| | IQKGGAMEPENKYLPELMAEKDSLDPSFTHAMQLLTAEIEKIQKG |
| | (muRIPK3-2xSrc.DM; TH1011 with no epitope tag) |
| 1337 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNCSEVQIGNYNSL |
| | VAPPRTTASSSAKYDQAQFGRGRGWQPFHKgvqvetispgdgrtfpkrgqtcvvhytgmledgkkVdssrdrnkpfkfmlgkqe |
| | virgweegvaqmsvgqrakltispdyaygatghpgiipphatlvfdvellklegvqvetispgdgrtfpkrgqtcvvhytgmledg |
| | kkVdssrdrnkpfkfmlgkqevirgweegvaqmsvgqrakltispdyaygatghpgiipphatlvfdvellkle |
| | (muRIPK3-2xFV; TH1012 with no epitope tag) |
| 1338 | MSCVKLWPSGAPAPLVSIEELENQELVGKGGFGTVFRAQHRKWGYDVAVKIVNSKAISREVKAMASLDNEFVLRLE |
| | GVIEKVNWDQDPKPALVTKFMENGSLSGLLQSQCPRPWPLLCRLLKEVVLGMFYLHDQNPVLLHRDLKPSNVLLDP |
| | ELHVKLADFGLSTFQGGSQSGTGSGEPGGTLGYLAPELFVNVNRKASTASDVYSFGILMWAVLAGREVELPTEPSLV |
| | YEAVCNRQNRPSLAELPQAGPETPGLEGLKELMQLCWSSEPKDRPSFQECLPKTDEVFQMVENNMNAAVSTVKDF |
| | LSQLRSSNRRFSIPESGQGGTEMDGFRRTIENQHSRNDVMVSEWLNKLNLEEPPSSVPKKCPSLTKRSRAQEEQVP |
| | QAWTAGTSSDSMAQPPQTPETSTFRNQMPSPTSTGTPSPGPRGNQGAERQGMNWSCRTPEPNPVTGRPLVNIY |
| | NCSGVQVGDNNYLTMQQTTALPTWGLAPSGKGRGLQHPPPVGSQEGPKDPEAWSRPQGWYNHSGKgvqvetisp |
| | gdgrtfpkrgqtcvvhytgmledgkkVdssrdrnkpfkfmlgkqevirgweegvaqmsvgqrakltispdyaygatghpgiippha |
| | tlvfdvellklegvqvetispgdgrtfpkrgqtcvvhytgmledgkkVdssrdrnkpfkfmlgkqevirgweegvaqmsvgqrakl |
| | tispdyaygatghpgiipphatlvfdvellkle |
| | (huRIPK3-2xFv; TH1013 with no epitope tag) |
| 1339 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNCSEVQIGNYNSL |
| | VAPPRTTASSSAKYDQAQFGRGRGWQPFHKggikkeieaikkeqeaikkkieaiekeiea |
| | (muRIPK3-IZ.Tri mer; TH1015 with no epitope tag) |
| 1340 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNCSEVQIGNYNSL |
| | VAPPRTTASSSAKYDQAQFGRGRGWQPFHKgggyipeaprdgqayvrkdgewvllstfl |
| | (muRIPK3-Foldon; TH1016 with no epitope tag) |
| 1341 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNCSEVQIGNYNSL |
| | VAPPRTTASSSAKYDQAQFGRGRGWQPFHKGSDGSGSGSGSITIRAAFLEKENTALRTEIAELEKEVGRCENIVSKYE |
| | TRYGPLGSDGSGSGSGSITIRAAFLEKENTALRTEIAELEKEVGRCENIVSKYETRYGPL |
| | (muRIPK3-2xEE; TH1017 with no epitope tag) |
| 1342 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNCSEVQIGNYNSL |
| | VAPPRTTASSSAKYDQAQFGRGRGWQPFHKGSDGSGSGSGSLEIRAAFLEKENTALRTRAAELRKRVGRCRNIVSKY |
| | ETRYGPLGSDGSGSGSGSLEIRAAFLEKENTALRTRAAELRKRVGRCRNIVSKYETRYGPL |
| | (muRIPK3-2xRR; TH1018 with no epitope tag) |
| 1343 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNCSEVQIGNYNSL |
| | VAPPRTTASSSAKYDQAQFGRGRGWQPFHKGSDGSGSGSGSITIRAAFLEKENTALRTEIAELEKEVGRCENIVSKYE |
| | TRYGPL |
| | (muRIPK3-EE; TH1019 with no epitope tag) |
| 1344 | MSSVKLWPTGASAVPLVSREELKKLEFVGKGGFGVVFRAHHRTWNHDVAVKIVNSKKISWEVKAMVNLRNENVLL |
| | LLGVTEDLQWDFVSGQALVTRFMENGSLAGLLQPECPRPWPLLCRLLQEVVLGMCYLHSLNPPLLHRDLKPSNILLD |
| | PELHAKLADFGLSTFQGGSQSGSGSGSGSRDSGGTLAYLDPELLFDVNLKASKASDVYSFGILVWAVLAGREAELVD |
| | KTSLIRETVCDRQSRPPLTELPPGSPETPGLEKLKELMIHCWGSQSENRPSFQDCEPKTNEVYNLVKDKVDAAVSEVK |
| | HYLSQHRSSGRNLSAREPSQRGTEMDCPRETMVSKMLDRLHLEEPSGPVPGKCPERQAQDTSVGPATPARTSSDP |
| | VAGTPQIPHTLPFRGTTPGPVFTETPGPHPQRNQGDGRHGTPWYPWTPPNPMTGPPALVFNNCSEVQIGNYNSL |
| | VAPPRTTASSSAKYDQAQFGRGRGWQPFHKGSDGSGSGSGSLEIRAAFLEKENTALRTRAAELRKRVGRCRNIVSKY |
| | ETRYGPL |
| | (muRIPK3-RR; TH1020 with no epitope tag) |
| 1345 | MGCVCSSNPEDDWMENGGIKKEIEAIKKEQEAIKKKIEAIEKEIEAGSGGGSGSGGGSDPFLVLLHSLSGSLSGNDLM |
| | ELKFLCRERVSKRKLERVQSGLDLFTVLLEQNDLERGHTGLLRELLASLRRHDLLQRLDDFEAGTATAAPPGEADLQV |
| | AFDIVCDNVGRDWKRLARELKVSEAKMDGIEEKYPRSLSERVRESLKVWKNAEKKNASVAGLVKALRTCRLNLVADL |
| | VEEAQESVSKSENMSPVLRDSTVSSSETP |
| | (Myr(Lck)-IZ-L-msFADD; TH3002 without epitope tag) |
| 1346 | MGCVCSSNPEDDWMENGGIKKEIEAIKKEQEAIKKKIEAIEKEIEAGSGGGSGSGGGSPGEEDLCAAFNVICDNVGK |
| | DWRRLARQLKVSDTKIDSIEDRYPRNLTERVRESLRIWKNTEKENATVAHLVGALRSCQMNLVADLVQEVQQARDL |
| | QNRSGAMSPMSWNS |
| | (Myr(Lck)-IZ-L-huFADD-DD; TH3003 without epitope tag) |
| 1347 | MGCVCSSNPEDDWMENGGIKKEIEAIKKEQEAIKKKIEAIEKEIEAGSGGGSGSGGGSPPGEADLQVAFDIVCDNVG |
| | RDWKRLARELKVSEAKMDGIEEKYPRSLSERVRESLKVWKNAEKKNASVAGLVKALRTCRLNLVADLVEEAQESVSK |
| | SENMSPVLRDSTVS |
| | (Myr(Lck)-IZ-L-msFADD-DD; TH3004 without epitope tag) |
| 1348 | MGQTVTTPLSLTLDHWGGIKKEIEAIKKEQEAIKKKIEAIEKEIEAGSGGGSGSGGGSDPFLVLLHSVSSSLSSSELTELK |
| | FLCLGRVGKRKLERVQSGLDLFSMLLEQNDLEPGHTELLRELLASLRRHDLLRRVDDFEAGAAAGAAPGEEDLCAAF |
| | NVICDNVGKDWRRLARQLKVSDTKIDSIEDRYPRNLTERVRESLRIWKNTEKENATVAHLVGALRSCQMNLVADLV |
| | QEVQQARDLQNRSGAMSPMSWNSDASTSEAS |
| | (Myr(MMSV)-IZ-L-huFADD; TH3005 without epitope tag) |
| 1349 | MGQTVTTPLSLTLDHWGGIKKEIEAIKKEQEAIKKKIEAIEKEIEAGSGGGSGSGGGSDPFLVLLHSLSGSLSGNDLMEL |
| | KFLCRERVSKRKLERVQSGLDLFTVLLEQNDLERGHTGLLRELLASLRRHDLLQRLDDFEAGTATAAPPGEADLQVAF |
| | DIVCDNVGRDWKRLARELKVSEAKMDGIEEKYPRSLSERVRESLKVWKNAEKKNASVAGLVKALRTCRLNLVADLVE |
| | EAQESVSKSENMSPVLRDSTVSSSETP |
| | (Myr(MMSV)-IZ-L-msFADD; TH3006 without epitope tag) |
| 1350 | MGQTVTTPLSLTLDHWGGIKKEIEAIKKEQEAIKKKIEAIEKEIEAGSGGGSGSGGGSPGEEDLCAAFNVICDNVGKD |
| | WRRLARQLKVSDTKIDSIEDRYPRNLTERVRESLRIWKNTEKENATVAHLVGALRSCQMNLVADLVQEVQQARDLQ |
| | NRSGAMSPMSWNS |
| | (Myr(MMSV)-IZ-L-huFADD-DD; TH3007 without epitope tag) |
| 1351 | MGQTVTTPLSLTLDHWGGIKKEIEAIKKEQEAIKKKIEAIEKEIEAGSGGGSGSGGGSPPGEADLQVAFDIVCDNVGR |
| | DWKRLARELKVSEAKMDGIEEKYPRSLSERVRESLKVWKNAEKKNASVAGLVKALRTCRLNLVADLVEEAQESVSKS |
| | ENMSPVLRDSTVS |
| | (Myr(MMSV)-IZ-L-msFADD-DD; TH3008 without epitope tag) |
| 1352 | MRMKQIEDKIEEILSKIYHIENEIARIKKLIGERAEGNHRKKPLKVLESLGKDFLTGVLDNLVEQNVLNWKEEEKKKYYD |
| | AKTEDKVRVMADSMQEKQRMAGQMLLQTFFNIDQISPNKKAHPNMEAGPPESGESTDALKLCPHEEFLRLCKERA |
| | EEIYPIKERNNRTRLALIICNTEFDHLPPRNGADFDITGMKELLEGLDYSVDVEENLTARDMESALRAFATRPEHKSSD |
| | STFLVLMSHGILEGICGTVHDEKKPDVLLYDTIFQIFNNRNCLSLKDKPKVIIVQACRGANRGELWVRDSPASLEVASS |
| | QSSENLEEDAVYKTHVEKDFIAFCSSTPHNVSWRDSTMGSIFITQLITCFQKYSWCCHLEEVFRKVQQSFETPRAKAQ |
| | MPTIERLSMTRYFYLFPGN |
| | (Caspase-4, full-length + IZ domain; P2006 without epitope tag) |
| 1353 | MRMKQIEDKIEEILSKIYHIENEIARIKKLIGERQISPNKKAHPNMEAGPPESGESTDALKLCPHEEFLRLCKERAEEIYPI |
| | KERNNRTRLALIICNTEFDHLPPRNGADFDITGMKELLEGLDYSVDVEENLTARDMESALRAFATRPEHKSSDSTFLVL |
| | MSHGILEGICGTVHDEKKPDVLLYDTIFQIFNNRNCLSLKDKPKVIIVQACRGANRGELWVRDSPASLEVASSQSSEN |
| | LEEDAVYKTHVEKDFIAFCSSTPHNVSWRDSTMGSIFITQLITCFQKYSWCCHLEEVFRKVQQSFETPRAKAQMPTIE |
| | RLSMTRYFYLFPGN |
| | (Caspase-4, N.del + IZ domain; P2009 without epitope tag) |
| 1354 | MRMKQLEDKIEELLSKIYHLENEIARLKKLIGERAEGNHRKKPLKVLESLGKDFLTGVLDNLVEQNVLNWKEEEKKKYY |
| | DAKTEDKVRVMADSMQEKQRMAGQMLLQTFFNIDQISPNKKAHPNMEAGPPESGESTDALKLCPHEEFLRLCKE |
| | RAEEIYPIKERNNRTRLALIICNTEFDHLPPRNGADFDITGMKELLEGLDYSVDVEENLTARDMESALRAFATRPEHKS |
| | SDSTFLVLMSHGILEGICGTVHDEKKPDVLLYDTIFQIFNNRNCLSLKDKPKVIIVQACRGANRGELWVRDSPASLEVA |
| | SSQSSENLEEDAVYKTHVEKDFIAFCSSTPHNVSWRDSTMGSIFITQLITCFQKYSWCCHLEEVFRKVQQSFETPRAK |
| | AQMPTIERLSMTRYFYLFPGN |
| | (Caspase-4, full-length + DM domain; P2012 without epitope tag) |
| 1355 | MRMKQLEDKIEELLSKIYHLENEIARLKKLIGERQISPNKKAHPNMEAGPPESGESTDALKLCPHEEFLRLCKERAEEIY |
| | PIKERNNRTRLALIICNTEFDHLPPRNGADFDITGMKELLEGLDYSVDVEENLTARDMESALRAFATRPEHKSSDSTFL |
| | VLMSHGILEGICGTVHDEKKPDVLLYDTIFQIFNNRNCLSLKDKPKVIIVQACRGANRGELWVRDSPASLEVASSQSSE |
| | NLEEDAVYKTHVEKDFIAFCSSTPHNVSWRDSTMGSIFITQLITCFQKYSWCCHLEEVFRKVQQSFETPRAKAQMPTI |
| | ERLSMTRYFYLFPGN |
| | (Caspase-4, N.del + DM domain; P2015 without epitope tag) |
| 1356 | MAEGNHRKKPLKVLESLGKDFLTGVLDNLVEQNVLNWKEEEKKKYYDAKTEDKVRVMADSMQEKQRMAGQMLL |
| | QTFFNIDQISPNKKAHPNMEAGPPESGESTDALKLCPHEEFLRLCKERAEEIYPIKERNNRTRLALIICNTEFDHLPPRN |
| | GADFDITGMKELLEGLDYSVDVEENLTARDMESALRAFATRPEHKSSDSTFLVLMSHGILEGICGTVHDEKKPDVLLY |
| | DTIFQIFNNRNCLSLKDKPKVIIVQACRGANRGELWVRDSPASLEVASSQSSENLEEDAVYKTHVEKDFIAFCSSTPHN |
| | VSWRDSTMGSIFITQLITCFQKYSWCCHLEEVFRKVQQSFETPRAKAQMPTIERLSMTRYFYLFPGN |
| | (Caspase-4, full-length wild-type; P2018 without epitope tag) |
| 1357 | MRMKQIEDKIEEILSKIYHIENEIARIKKLIGERAEDSGKKKRRKNFEAMFKGILQSGLDNFVINHMLKNNVAGQTSIQ |
| | TLVPNTDQKSTSVKKDNHKKKTVKMLEYLGKDVLHGVFNYLAKHDVLTLKEEEKKKYYDTKIEDKALILVDSLRKNRV |
| | AHQMFTQTLLNMDQKITSVKPLLQIEAGPPESAESTNILKLCPREEFLRLCKKNHDEIYPIKKREDRRRLALIICNTKFD |
| | HLPARNGAHYDIVGMKRLLQGLGYTVVDEKNLTARDMESVLRAFAARPEHKSSDSTFLVLMSHGILEGICGTAHKKK |
| | KPDVLLYDTIFQIFNNRNCLSLKDKPKVIIVQACRGEKHGELWVRDSPASLALISSQSSENLEADSVCKIHEEKDFIAFCS |
| | STPHNVSWRDRTRGSIFITELITCFQKYSCCCHLMEIFRKVQKSFEVPQAKAQMPTIERATLTRDFYLFPGN |
| | (Caspase-5, full-length + IZ domain; P2007 without epitope tag) |
| 1358 | MRMKQIEDKIEEILSKIYHIENEIARIKKLIGERDQKITSVKPLLQIEAGPPESAESTNILKLCPREEFLRLCKKNHDEIYPIK |
| | KREDRRRLALIICNTKFDHLPARNGAHYDIVGMKRLLQGLGYTVVDEKNLTARDMESVLRAFAARPEHKSSDSTFLVL |
| | MSHGILEGICGTAHKKKKPDVLLYDTIFQIFNNRNCLSLKDKPKVIIVQACRGEKHGELWVRDSPASLALISSQSSENLE |
| | ADSVCKIHEEKDFIAFCSSTPHNVSWRDRTRGSIFITELITCFQKYSCCCHLMEIFRKVQKSFEVPQAKAQMPTIERATL |
| | TRDFYLFPGN |
| | (Caspase-5, N.del + IZ domain; P2010 without epitope tag) |
| 1359 | MRMKQLEDKIEELLSKIYHLENEIARLKKLIGERAEDSGKKKRRKNFEAMFKGILQSGLDNFVINHMLKNNVAGQTSI |
| | QTLVPNTDQKSTSVKKDNHKKKTVKMLEYLGKDVLHGVFNYLAKHDVLTLKEEEKKKYYDTKIEDKALILVDSLRKNR |
| | VAHQMFTQTLLNMDQKITSVKPLLQIEAGPPESAESTNILKLCPREEFLRLCKKNHDEIYPIKKREDRRRLALIICNTKFD |
| | HLPARNGAHYDIVGMKRLLQGLGYTVVDEKNLTARDMESVLRAFAARPEHKSSDSTFLVLMSHGILEGICGTAHKKK |
| | KPDVLLYDTIFQIFNNRNCLSLKDKPKVIIVQACRGEKHGELWVRDSPASLALISSQSSENLEADSVCKIHEEKDFIAFCS |
| | STPHNVSWRDRTRGSIFITELITCFQKYSCCCHLMEIFRKVQKSFEVPQAKAQMPTIERATLTRDFYLFPGN |
| | (Caspase-5, full-length + DM domain; P2013 without epitope tag) |
| 1360 | MRMKQLEDKIEELLSKIYHLENEIARLKKLIGERDQKITSVKPLLQIEAGPPESAESTNILKLCPREEFLRLCKKNHDEIYPI |
| | KKREDRRRLALIICNTKFDHLPARNGAHYDIVGMKRLLQGLGYTVVDEKNLTARDMESVLRAFAARPEHKSSDSTFL |
| | VLMSHGILEGICGTAHKKKKPDVLLYDTIFQIFNNRNCLSLKDKPKVIIVQACRGEKHGELWVRDSPASLALISSQSSE |
| | NLEADSVCKIHEEKDFIAFCSSTPHNVSWRDRTRGSIFITELITCFQKYSCCCHLMEIFRKVQKSFEVPQAKAQMPTIER |
| | ATLTRDFYLFPGN |
| | (Caspase-5, N.del + DM domain; P2016 without epitope tag) |
| 1361 | MRMKQLEDKIEELLSKIYHLENEIARLKKLIGERDQKITSVKPLLQIEAGPPESAESTNILKLCPREEFLRLCKKNHDEIYPI |
| | KKREDRRRLALIICNTKFDHLPARNGAHYDIVGMKRLLQGLGYTVVDEKNLTARDMESVLRAFAARPEHKSSDSTFL |
| | VLMSHGILEGICGTAHKKKKPDVLLYDTIFQIFNNRNCLSLKDKPKVIIVQACRGEKHGELWVRDSPASLALISSQSSE |
| | NLEADSVCKIHEEKDFIAFCSSTPHNVSWRDRTRGSIFITELITCFQKYSCCCHLMEIFRKVQKSFEVPQAKAQMPTIER |
| | ATLTRDFYLFPGN |
| | (Caspase-5, full-length wild-type; P2019 without epitope tag) |
| 1362 | MRMKQIEDKIEEILSKIYHIENEIARIKKLIGERAENKHPDKPLKVLEQLGKEVLTEYLEKLVQSNVLKLKEEDKQKFNNA |
| | ERSDKRWVFVDAMKKKHSKVGEMLLQTFFSVDPGSHHGEANLEMEEPEESLNTLKLCSPEEFTRLCREKTQEIYPIKE |
| | ANGRTRKALIICNTEFKHLSLRYGANFDIIGMKGLLEDLGYDVVVKEELTAEGMESEMKDFAALSEHQTSDSTFLVLM |
| | SHGTLHGICGTMHSEKTPDVLQYDTIYQIFNNCHCPGLRDKPKVIIVQACRGGNSGEMWIRESSKPQLCRGVDLPR |
| | NMEADAVKLSHVEKDFIAFYSTTPHHLSYRDKTGGSYFITRLISCFRKHACSCHLFDIFLKVQQSFEKASIHSQMPTIDR |
| | ATLTRYFYLFPGN |
| | (Caspase-11, full-length + IZ domain; P2005 without epitope tag) |
| 1363 | MRMKQIEDKIEEILSKIYHIENEIARIKKLIGERPGSHHGEANLEMEEPEESLNTLKLCSPEEFTRLCREKTQEIYPIKEAN |
| | GRTRKALIICNTEFKHLSLRYGANFDIIGMKGLLEDLGYDVVVKEELTAEGMESEMKDFAALSEHQTSDSTFLVLMSH |
| | GTLHGICGTMHSEKTPDVLQYDTIYQIFNNCHCPGLRDKPKVIIVQACRGGNSGEMWIRESSKPQLCRGVDLPRNM |
| | EADAVKLSHVEKDFIAFYSTTPHHLSYRDKTGGSYFITRLISCFRKHACSCHLFDIFLKVQQSFEKASIHSQMPTIDRATL |
| | TRYFYLFPGN |
| | (Caspase-11, N.del + IZ domain; P2008 without epitope tag) |
| 1364 | MRMKQIEDKIEEILSKIYHIENEIARIKKLIGERPGSHHGEANLEMEEPEESLNTLKLCSPEEFTRLCREKTQEIYPIKEAN |
| | GRTRKALIICNTEFKHLSLRYGANFDIIGMKGLLEDLGYDVVVKEELTAEGMESEMKDFAALSEHQTSDSTFLVLMSH |
| | GTLHGICGTMHSEKTPDVLQYDTIYQIFNNCHCPGLRDKPKVIIVQACRGGNSGEMWIRESSKPQLCRGVDLPRNM |
| | EADAVKLSHVEKDFIAFYSTTPHHLSYRDKTGGSYFITRLISCFRKHACSCHLFDIFLKVQQSFEKASIHSQMPTIDRATL |
| | TRYFYLFPGN |
| | (Caspase-11, full-length + DM domain; P2011 without epitope tag) |
| 1365 | MRMKQLEDKIEELLSKIYHLENEIARLKKLIGERAENKHPDKPLKVLEQLGKEVLTEYLEKLVQSNVLKLKEEDKQKENN |
| | AERSDKRWVFVDAMKKKHSKVGEMLLQTFFSVDPGSHHGEANLEMEEPEESLNTLKLCSPEEFTRLCREKTQEIYPI |
| | KEANGRTRKALIICNTEFKHLSLRYGANFDIIGMKGLLEDLGYDVVVKEELTAEGMESEMKDFAALSEHQTSDSTFLV |
| | LMSHGTLHGICGTMHSEKTPDVLQYDTIYQIFNNCHCPGLRDKPKVIIVQACRGGNSGEMWIRESSKPQLCRGVDL |
| | PRNMEADAVKLSHVEKDFIAFYSTTPHHLSYRDKTGGSYFITRLISCFRKHACSCHLFDIFLKVQQSFEKASIHSQMPTI |
| | DRATLTRYFYLFPGN |
| | (Caspase-11, N.del + DM domain; P2014 without epitope tag) |
| 1366 | MAENKHPDKPLKVLEQLGKEVLTEYLEKLVQSNVLKLKEEDKQKFNNAERSDKRWVFVDAMKKKHSKVGEMLLQT |
| | FFSVDPGSHHGEANLEMEEPEESLNTLKLCSPEEFTRLCREKTQEIYPIKEANGRTRKALIICNTEFKHLSLRYGANFDII |
| | GMKGLLEDLGYDVVVKEELTAEGMESEMKDFAALSEHQTSDSTFLVLMSHGTLHGICGTMHSEKTPDVLQYDTIYQ |
| | IFNNCHCPGLRDKPKVIIVQACRGGNSGEMWIRESSKPQLCRGVDLPRNMEADAVKLSHVEKDFIAFYSTTPHHLSY |
| | RDKTGGSYFITRLISCFRKHACSCHLFDIFLKVQQSFEKASIHSQMPTIDRATLTRYFYLFPGN |
| | (Caspase-11, full-length wild-type; P2017 without epitope tag) |
| 1367 | GSAFERVVRRVVQELDHGGEFIPVTSLQSSTGFQPYCLVVRKPSSSWFWKPRYKCVNLSIKDILEPDAAEPDVQRGR |
| | SFHFYDAMDGQIQGSVELAAPGQAKIAGGAAVSDSSSTSMNVYSLSVDPNTWQTLLHERHLRQPEHKVLQQLRSR |
| | GDNVYVVTEVLQTQKEVEVTRTHKREGSGRFSLPGATCLQGEGQGHLSQKKTVTIPSGSTLAFRVAQLVIDSDLDVL |
| | LFPDKKQRTFQPPATGHKRSTSEGAWPQLPSGLSMMRCLHNFLTDGVPAEGAFTEDFQGLRAEVETISKELELLDRE |
| | LCQLLLEGLEGVLRDQLALRALEEALEQGQSLGPVEPLDGPAGAVLECLVLSSGMLVPELAIPVVYLLGALTMLSETQ |
| | HKLLAEALESQTLLGPLELVGSLLEQSAPWQERSTMSLPPGLLGNSWGEGAPAWVLLDECGLELGEDTPHVCWEPQ |
| | AQGRMCALYASLALLSGLSQEPH |
| | (human GSDMD; SAW001 with no epitope tag) |
| 1368 | GSAFERVVRRVVQELDHGGEFIPVTSLQSSTGFQPYCLVVRKPSSSWFWKPRYKCVNLSIKDILEPDAAEPDVQRGR |
| | SFHFYDAMDGQIQGSVELAAPGQAKIAGGAAVSDSSSTSMNVYSLSVDPNTWQTLLHERHLRQPEHKVLQQLRSR |
| | GDNVYVVTEVLQTQKEVEVTRTHKREGSGRFSLPGATCLQGEGQGHLSQKKTVTIPSGSTLAFRVAQLVIDSDLDVL |
| | LFPDKKQRTFQPPATGHKRSTSEGAWPQLPSGLSMMRCLHNFLTD |
| | (human GSDMD(1-275); SAW002 with no epitope tag) |
| 1369 | GVPAEGAFTEDFQGLRAEVETISKELELLDRELCQLLLEGLEGVLRDQLALRALEEALEQGQSLGPVEPLDGPAGAVLE |
| | CLVLSSGMLVPELAIPVVYLLGALTMLSETQHKLLAEALESQTLLGPLELVGSLLEQSAPWQERSTMSLPPGLLGNSW |
| | GEGAPAWVLLDECGLELGEDTPHVCWEPQAQGRMCALYASLALLSGLSQEPH |
| | (human GSDMD(276-484); SAW003 with no epitope tag) |
| 1370 | PSAFEKVVKNVIKEVSGSRGDLIPVDSLRNSTSFRPYCLLNRKFSSSRFWKPRYSCVNLSIKDILEPSAPEPEPECFGSFK |
| | VSDVVDGNIQGRVMLSGMGEGKISGGAAVSDSSSASMNVCILRVTQKTWETMQHERHLQQPENKILQQLRSRGD |
| | DLFVVTEVLQTKEEVQITEVHSQEGSGQFTLPGALCLKGEGKGHQSRKKMVTIPAGSILAFRVAQLLIGSKWDILLVS |
| | DEKQRTFEPSSGDRKAVGQRHHGLNVLAALCSIGKQLSLLSDGIDEEELIEAADFQGLYAEVKACSSELESLEMELRQ |
| | QILVNIGKILQDQPSMEALEASLGQGLCSGGQVEPLDGPAGCILECLVLDSGELVPELAAPIFYLLGALAVLSETQQQL |
| | LAKALETTVLSKQLELVKHVLEQSTPWQEQSSVSLPTVLLGDCWDEKNPTWVLLEECGLRLQVESPQVHWEPTSLIP |
| | TSALYASLFLLSSLGQKPC |
| | (mouse GSDMD; SAW004 with no epitope tag) |
| 1371 | PSAFEKVVKNVIKEVSGSRGDLIPVDSLRNSTSFRPYCLLNRKFSSSRFWKPRYSCVNLSIKDILEPSAPEPEPECFGSFK |
| | VSDVVDGNIQGRVMLSGMGEGKISGGAAVSDSSSASMNVCILRVTQKTWETMQHERHLQQPENKILQQLRSRGD |
| | DLFVVTEVLQTKEEVQITEVHSQEGSGQFTLPGALCLKGEGKGHQSRKKMVTIPAGSILAFRVAQLLIGSKWDILLVS |
| | DEKQRTFEPSSGDRKAVGQRHHGLNVLAALCSIGKQLSLLSD |
| | (mouse GSDMD(1-276); SAW005 with no epitope tag) |
| 1372 | GIDEEELIEAADFQGLYAEVKACSSELESLEMELRQQILVNIGKILQDQPSMEALEASLGQGLCSGGQVEPLDGPAGCI |
| | LECLVLDSGELVPELAAPIFYLLGALAVLSETQQQLLAKALETTVLSKQLELVKHVLEQSTPWQEQSSVSLPTVLLGDC |
| | WDEKNPTWVLLEECGLRLQVESPQVHWEPTSLIPTSALYASLFLLSSLGQKPC |
| | (mouse GSDMD(277-487); SAW006 with no epitope tag) |
| 1373 | MKMASTRCKLARYLEDLEDVDLKKFKMHLEDYPPQKGCIPLPRGQTEKADHVDLATLMIDFNGEEKAWAMAVWI |
| | FAAINRRDLYEKAKRDEPKWGSDNARVSNPTVICQEDSIEEEWMGLLEYLSRISICKMKKDYRKKYRKYVRSRFQCIE |
| | DRNARLGESVSLNKRYTRLRLIKEHRSQQEREQELLAIGKTKTCESPVSPIKMELLFDPDDEHSEPVHTVVFQGAAGIG |
| | KTILARKMMLDWASGTLYQDRFDYLFYIHCREVSLVTQRSLGDLIMSCCPDPNPPIHKIVRKPSRILFLMDGFDELQG |
| | AFDEHIGPLCTDWQKAERGDILLSSLIRKKLLPEASLLITTRPVALEKLQHLLDHPRHVEILGFSEAKRKEYFFKYFSDEA |
| | QARAAFSLIQENEVLFTMCFIPLVCWIVCTGLKQQMESGKSLAQTSKTTTAVYVFFLSSLLQPRGGSQEHGLCAHLW |
| | GLCSLAADGIWNQKILFEESDLRNHGLQKADVSAFLRMNLFQKEVDCEKFYSFIHMTFQEFFAAMYYLLGGGSGGG |
| | SGGIKKEIEAIKKEQEAIKKKIEAIEKEIEA |
| | (hu.caNLRP3(PYD_NACHT_IZ); P3005 without epitope tag) |
| 1374 | MTSVRCKLAQYLEDLEDVDLKKFKMHLEDYPPEKGCIPVPRGQMEKADHLDLATLMIDFNGEEKAWAMAVWIFA |
| | AINRRDLWEKAKKDQPEWNDTCTSHSSMVCQEDSLEEEWMGLLGYLSRISICKKKKDYCKMYRRHVRSRFYSIKDR |
| | NARLGESVDLNSRYTQLQLVKEHPSKQEREHELLTIGRTKMRDSPMSSLKLELLFEPEDGHSEPVHTVVFQGAAGIGK |
| | TILARKIMLDWALGKLFKDKFDYLFFIHCREVSLRTPRSLADLIVSCWPDPNPPVCKILRKPSRILFLMDGFDELQGAFD |
| | EHIGEVCTDWQKAVRGDILLSSLIRKKLLPKASLLITTRPVALEKLQHLLDHPRHVEILGFSEAKRKEYFFKYFSNELQAR |
| | EAFRLIQENEVLFTMCFIPLVCWIVCTGLKQQMETGKSLAQTSKTTTAVYVFFLSSLLQSRGGIEEHLFSDYLQGLCSL |
| | AADGIWNQKILFEECDLRKHGLQKTDVSAFLRMNVFQKEVDCERFYSFSHMTFQEFFAAMYYLLGGGSGGGSGGI |
| | KKEIEAIKKEQEAIKKKIEAIEKEIEA |
| | (mu.caNLRP3(PYD_NACHT_IZ); P3007 without epitope tag) |
| 1375 | MAKTLGDHLLNTLEELLPYDFEKFKFKLQNTSLEKGHSKIPRGHMQMARPVKLASLLITYYGEEYAVRLTLQILRATNQ |
| | RQLAEELRKATGTEHLIEENRVGGSVQSSVENKAKSVKVPDVPEGDGTQQNNDESDTLPSSQAEVGKGPQKKSLTK |
| | RKDQRGPESLDSQTKPWTRSTAPLYRRTQGTQSPGDKESTASAQLRRNVSSAGRLQGLYNNAPGRRESKKAEVYVY |
| | LPSGKKRPRSLEITTYSREGEPPNSEVLPTQEETRNGSLIRMRTATLNGRTTGALEKGTGIPEHSMVLDEKTFRNMSSK |
| | TSLIGEERCPTSWTENGNGSPETTESSGETAGSILSDPEVPLSLCEKPAKTPEDPASLGQAACEGRSQDKAVCPLCHT |
| | QEGDLRGDTCVQSSCSCSIAPGDPKASGRCSICFQCQGLLARKSCEAQSPQSLPQCPRHMKQVLLLFCEDHREPICLI |
| | CRLSLEHQGHRVRPIEEAALEYKEQIREQLERLREMRGYVEEHRLQGDKKTDDFLKQTEIQKQKISCPLEKLYQLLEKQ |
| | EQLFVTWLQELSQTISKVRETYYTRVSLLDEMIEELEAKQDQPEWDLMQDIGITLHRAKMMSASELLDTPPGVKEKL |
| | HLLYQKSKSVEKNMQCFSEMLSSEMAFSASDVAKWEGRQPSATQVQGLVPTVHLKCDGAHTQDCDVVFYPEREA |
| | GGSEPKDYLHPQPAQDTPELHEIHSRNNKRKFKSFLKWKPSFSRTDWRLRTCCYRDLDQAAAHPNLIFSMI |
| | (muPYRIN-B30.2(V726A); P3002 without epitope tag) |
| 1376 | MAKTLGDHLLNTLEELLPYDFEKFKFKLQNTSLEKGHSKIPRGHMQMARPVKLASLLITYYGEEYAVRLTLQILRATNQ |
| | RQLAEELRKATGTEHLIEENRVGGSVQSSVENKAKSVKVPDVPEGDGTQQNNDESDTLPSSQAEVGKGPQKKSLTK |
| | RKDQRGPESLDSQTKPWTRSTAPLYRRTQGTQSPGDKESTASAQLRRNVSSAGRLQGLYNNAPGRRESKKAEVYVY |
| | LPSGKKRPRSLEITTYSREGEPPNSEVLPTQEETRNGSLIRMRTATLNGRTTGALEKGTGIPEHSMVLDEKTFRNMSSK |
| | TSLIGEERCPTSWTENGNGSPETTESSGETAGSILSDPEVPLSLCEKPAKTPEDPASLGQAACEGRSQDKAVCPLCHT |
| | QEGDLRGDTCVQSSCSCSIAPGDPKASGRCSICFQCQGLLARKSCEAQSPQSLPQCPRHMKQVLLLFCEDHREPICLI |
| | CRLSLEHQGHRVRPIEEAALEYKEQIREQLERLREMRGYVEEHRLQGDKKTDDFLKQTEIQKQKISCPLEKLYQLLEKQ |
| | EQLFVTWLQELSQTISKVRETYYTRVSLLDEMIEELEAKQDQPEWDLMQDIGITLHRAKMMSASELLDTPPGVKEKL |
| | HLLYQKSKSVEKNMQCFSEMLSSEMAFSASDVAKWEGRQPSATQVQGLVPTVHLKCDGAHTQDCDVVFYPEREA |
| | GGSEPKDYLHPQPAQDTPELHEIHSRNNKRKFKSFLKWKPSFSRTDWRLRTCCYRDLDQAAAHPNLIFSMISEMEM |
| | FNVPELIGAQAHAVNVILDAETAYPNLIFSDDLKSVRLGNKWERLPDGPQRFDSCIIVLGSPSFLSGRRYWEVEVGDK |
| | TAWILGACKTSISRKGNMTLSPENGYWVVIMMKENEYQASSVPPTRLLIKEPPKRVGIFVDYRVGSISFYNVTARSHI |
| | YTFASCSFSGPLQPIFSPGTRDGGKNTAPLTICPVGGQGPD |
| | (muPYRIN-B30.2; P3004 without epitope tag) |
| 1377 | MGGIKKEIEAIKKEQEAIKKKIEAIEKEIEAGSGAAPAGIQAPPQSAAKPGLHFIDQHRAALIARVTNVEWLLDALYGK |
| | VLTDEQYQAVRAEPTNPSKMRKLFSFTPAWNWTCKDLLLQALRESQSYLVEDLERS |
| | (hu.caASC(IZ_CARD); P3006 without epitope tag) |
| 1378 | MGGIKKEIEAIKKEQEAIKKKIEAIEKEIEAGSGAVAAAASVPAQSTARTGHFVDQHRQALIARVTEVDGVLDALHGS |
| | VLTEGQYQAVRAETTSQDKMRKLFSFVPSWNLTCKDSLLQALKEIHPYLVMDLEQS |
| | (mu.caASC(IZ_CARD); P3008 without epitope tag) |
| 1379 | MSCVKLWPSGAPAPLVSIEELENQELVGKGGFGTVFRAQHRKWGYDVAVKIVNSKAISREVKAMASLDNEFVLRLE |
| | GVIEKVNWDQDPKPALVTKFMENGSLSGLLQSQCPRPWPLLCRLLKEVVLGMFYLHDQNPVLLHRDLKPSNVLLDP |
| | ELHVKLADFGLSTFQGGSQSGTGSGEPGGTLGYLAPELFVNVNRKASTASDVYSFGILMWAVLAGREVELPTEPSLV |
| | YEAVCNRQNRPSLAELPQAGPETPGLEGLKELMQLCWSSEPKDRPSFQECLPKTDEVFQMVENNMNAAVSTVKDF |
| | LSQLRSSNRRFSIPESGQGGTEMDGFRRTIENQHSRNDVMVSEWLNKLNLEEPPSSVPKKCPSLTKRSRAQEEQVP |
| | QAWTAGTSSDSMAQPPQTPETSTFRNQMPSPTSTGTPSPGPRGNQGAERQGMNWSCRTPEPNPVTGRPLVNIY |
| | NCSGVQVGDNNYLTMQQTTALPTWGLAPSGKGRGLQHPPPVGSQEGPKDPEAWSRPQGWYNHSGK |
| | (huRIPK3; TH1014 with no epitope tag) |
| 1380 | GGGAAATAAGAGAGAAAAGAAGAGTAAGAAGAAATATAAGAGCCACC |
| | (5′UTR) |
| 1381 | CCGCCGCCGCCG |
| | (GC-Rich RNA Element) |
| 1382 | CCGCCGCCGCCGCCG |
| | (GC Rich RNA Element) |
| 1383 | CCCCGGCGCC |
| | (GC Rich RNA Element) |
| 1384 | GGGAAATAAGAGAGAAAAGAAGAGTAAGAAGAAATATAAGA |
| | (5′UTR) |
| 1385 | GGGAAATAAGAGAGAAAAGAAGAGTAAGAAGAAATATAAGACCCCGGCGCCGCCACC |
| | (5′UTR-V1) |
| 1386 | GGGAAATAAGAGAGAAAAGAAGAGTAAGAAGAAATATAAGACCCCGGCGCCACC |
| | (5′UTR-V2) |
| 1387 | MEHDLERGPPGPRRPPRGPPLSSSLGLALLLLLLALLFWLYIVMSDWTGGALLVLYSFALMLIIIILIIFIFRRDLLCPLGAL |
| | CILLLMITLLLIALWNLHGQALFLGIVLFIFGCLLVLGIWIYLLEMLWRLGATIWQLLAFFLAFFLDLILLIIALYLQQNWW |
| | TLLVDLLWLLLFLAILIWMYYHGQRPFAEDKTYKYICRNFSNFCNVDVVEILPYLPCLTARDQDRLRATCTLSGNRDTL |
| | WHLFNTLQRRPGWVEYFIAALRGCELVDLADEVASVYQSYQPRTSDRPPDPLEPPSLPAERPGPPTPAAAHSIPYNS |
| | CREKEPSYPMPVQETQAPESPGENSEQALQTLSPRAIPRNPDGGPLESSSDLAALSPLTSSGHQEQDTELGSTHTAG |
| | ATSSLTPSRGPVSPSVSFQPLARSTPRASRLPGPTGSVVSTGTSFSSSSPGLASAGAAEGKQGAESDQAEPIICSSGAE |
| | APANSLPSKVPTTLMPVNTVALKVPANPASVSTVPSKLPTSSKPPGAVPSNALTNPAPSKLPINSTRAGMVPSKVPTS |
| | MVLTKVSASTVPTDGSSRNEETPAAPTPAGATGGSSAWLDSSSENRGLGSELSKPGVLASQVDSPFSGCFEDLAISAS |
| | TSLGMGPCHGPEENEYKSEGTFGIHVAENPSIQLLEGNPGPPADPDGGPRPQADRKFQEREVPCHRPSPGALWLQ |
| | VAVTGVLVVTLLVVLYRRRLH |
| | (Human MAVS) |
| 1388 | ATIGTAMYK (EBV BRLF1) |
| 1389 | SIIPSGPLK (FLU) |
| 1390 | AVDLSHFLK (HIV NEF) |
| 1391 | AVFDRKSDAK (EBV) |
| 1392 | YVNVNMGLK (HBV CORE ANUIGEN) |
| 1393 | RVCEKMALY (HCV) |
| 1394 | KLGGALQAK (CMV) |
| 1395 | ATGAGAATGAAGCAGCTGGAGGACAAGATCGAGGAGCTGCTGAGCAAGATCTACCACCTGGAGAACGAGATC |
| | GCCAGACTGAAGAAGCTGATCGGCGAGGCCGACCAGACCAGCGGCAACTACCTGAACATGCAGGACAGCCAG |
| | GGCGTGCTGAGCAGCTTCCCCGCCCCCCAGGCCGTGCAGGACAACCCCGCCATGCCCACCAGCAGCGGCAGCG |
| | AGGGCAACGTGAAGCTGTGCAGCCTGGAGGAGGCCCAGAGAATCTGGAAGCAGAAGAGCGCCGAGATCTAC |
| | CCCATCATGGACAAGAGCAGCAGAACCAGACTGGCCCTGATCATCTGCAACGAGGAGTTCGACAGCATCCCCA |
| | GAAGAACCGGCGCCGAGGTGGACATCACCGGCATGACCATGCTGCTGCAGAACCTGGGCTACAGCGTGGACG |
| | TGAAGAAGAACCTGACCGCCAGCGACATGACCACCGAGCTGGAGGCCTTCGCCCACAGACCCGAGCACAAGA |
| | CCAGCGACAGCACCTTCCTGGTGTTCATGAGCCACGGCATCAGAGAGGGCATCTGCGGCAAGAAGCACAGCG |
| | AGCAGGTGCCCGACATCCTGCAGCTGAACGCCATCTTCAACATGCTGAACACCAAGAACTGCCCCAGCCTGAA |
| | GGACAAGCCCAAGGTGATCATCATCCAGGCCTGCAGAGGCGACAGCCCCGGCGTGGTGTGGTTCAAGGACAG |
| | CGTGGGCGTGAGCGGCAACCTGAGCCTGCCCACCACCGAGGAGTTCGAGGACGACGCCATCAAGAAGGCCCA |
| | CATCGAGAAGGACTTCATCGCCTTCTGCAGCAGCACCCCCGACAACGTGAGCTGGAGACACCCCACCATGGGC |
| | AGCGTGTTCATCGGCAGACTGATCGAGCACATGCAGGAGTACGCCTGCAGCTGCGACGTGGAGGAGATCTTCA |
| | GAAAGGTGAGATTCAGCTTCGAGCAGCCCGACGGCAGAGCCCAGATGCCCACCACCGAGAGAGTGACCCTGA |
| | CCAGATGCTTCTACCTGTTCCCCGGCCAC |
| | DM_hsCASP1 (self-activating human Caspase 1); P2025 without epitope tag) |
| 1396 | ATGAGAATGAAGCAGCTGGAGGACAAGATCGAGGAGCTGCTGAGCAAGATCTATCACCTGGAGAACGAGATC |
| | GCCAGACTGAAGAAGCTGATCGGCGAGAGACAGATCAGCCCCAACAAGAAGGCCCACCCCAACATGGAGGCC |
| | GGACCGCCTGAGAGCGGCGAGAGCACCGACGCCCTGAAGCTGTGCCCCCACGAGGAGTTCCTGAGACTGTGC |
| | AAGGAGAGAGCCGAGGAGATCTACCCCATCAAGGAGAGAAACAACAGAACCAGACTGGCCCTGATCATCTGC |
| | AACACCGAGTTCGACCACCTGCCCCCCAGAAACGGCGCCGACTTCGACATCACCGGCATGAAGGAGCTGCTGG |
| | AGGGCCTGGACTACAGCGTGGACGTGGAGGAGAACCTGACCGCCAGAGACATGGAGAGCGCCCTGAGAGCC |
| | TTCGCCACCAGACCCGAGCACAAGAGCAGCGACAGCACCTTCCTGGTGCTGATGAGCCACGGCATCCTGGAGG |
| | GCATCTGCGGCACCGTGCACGACGAGAAGAAGCCCGACGTGCTGCTGTACGACACCATCTTCCAGATCTTCAAC |
| | AACAGAAACTGCCTGAGCCTGAAGGACAAGCCCAAGGTGATCATCGTGCAGGCCTGCAGAGGCGCCAACAGA |
| | GGCGAGCTGTGGGTGAGAGACAGCCCCGCCAGCCTGGAGGTGGCCAGCAGCCAGAGCAGCGAGAACCTGGA |
| | GGAGGACGCCGTGTACAAGACCCACGTGGAGAAGGACTTCATCGCCTTCTGCAGCAGCACCCCCCACAACGTG |
| | AGCTGGAGAGACAGCACCATGGGCAGCATCTTCATCACCCAGCTGATAACCTGCTTCCAGAAGTACAGCTGGT |
| | GCTGCCACCTGGAGGAGGTGTTCAGAAAGGTGCAGCAGAGCTTCGAGACCCCCAGAGCCAAGGCCCAGATGC |
| | CCACCATCGAGAGACTGAGCATGACCAGATACTTCTACCTGTTCCCCGGCAAC |
| | (Caspase-4, N.del + DM domain; P2015 without epitope tag) |
| 1397 | ATGTCGTGGTCCCCCTCACTTACTACTCAAACTTGCGGCGCCTGGGAAATGAAGGAAAGACTCGGTACCGGGG |
| | GATTTGGAAACGTGATCCGGTGGCACAACCAAGAAACCGGAGAGCAAATTGCGATCAAGCAGTGTAGACAGG |
| | AACTGAGCCCTCGGAACAGAGAGCGGTGGTGCCTGGAGATTCAGATTATGCGCCGGCTGACCCATCCGAACGT |
| | GGTGGCTGCCAGGGATGTCCCGGAGGGCATGCAGAACCTGGCCCCTAACGACCTCCCACTCCTGGCCATGGAA |
| | TACTGCCAGGGTGGCGATCTGCGGAAGTACCTTAACCAATTCGAAAACTGCTGTGGACTCAGGGAAGGGGCCA |
| | TTCTGACTCTCTTGTCGGACATCGCCAGCGCCCTGAGATACCTCCACGAGAACAGAATCATCCATCGCGATCTG |
| | AAGCCGGAGAACATTGTGCTGCAACAGGGCGAACAGCGGCTGATCCACAAAATCATTGATCTCGGATATGCCA |
| | AGGAACTGGACCAGGGCGAACTCTGCACCGAATTCGTGGGCACTCTCCAGTACCTGGCACCCGAGTTGCTGGA |
| | GCAGCAGAAGTACACCGTCACCGTCGACTACTGGTCCTTCGGAACCCTCGCATTCGAATGTATCACTGGCTTCC |
| | GCCCTTTCCTGCCTAACTGGCAGCCTGTGCAGTGGCATTCGAAGGTCCGGCAGAAATCGGAGGTGGACATCGT |
| | GGTGTCCGAGGATCTGAACGGCACAGTGAAGTTCTCCTCCTCACTGCCTTACCCCAACAACCTCAACTCCGTGCT |
| | GGCCGAACGGCTGGAAAAGTGGCTCCAGCTTATGCTGATGTGGCATCCACGCCAGCGGGGTACTGATCCGACC |
| | TACGGTCCGAACGGGTGCTTCAAGGCCCTGGACGACATACTGAACCTCAAGCTCGTGCACATCCTCAATATGGT |
| | GACCGGCACGATCCATACTTACCCCGTCACCGAGGACGAATCGTTGCAGTCACTGAAGGCTCGGATCCAGCAG |
| | GACACCGGGATTCCCGAAGAGGACCAGGAACTTCTGCAGGAAGCGGGACTGGCGTTGATCCCCGACAAGCCT |
| | GCCACCCAGTGCATCTCTGACGGGAAGCTGAATGAAGGTCACACCCTGGATATGGACCTTGTGTTCCTGTTCGA |
| | CAATTCCAAGATCACCTACGAGACTCAGATTAGCCCTAGGCCTCAGCCGGAATCCGTGTCGTGCATCCTGCAAG |
| | AACCGAAGCGGAATCTGGCGTTCTTTCAACTGCGGAAAGTGTGGGGCCAAGTCTGGCACAGCATTCAGACACT |
| | GAAGGAGGATTGCAACCGGCTGCAGCAAGGACAGCGCGCCGCTATGATGAATCTGCTGCGCAACAATTCCTGC |
| | CTCTCAAAAATGAAGAACTCCATGGCCTCGATGTCCCAGCAATTGAAGGCCAAGCTGGATTTCTTCAAGACCTC |
| | GATCCAGATCGACCTGGAAAAGTACAGCGAGCAGACCGAGTTCGGAATCACCTCCGACAAGCTGCTGTTGGCA |
| | TGGCGGGAGATGGAACAAGCGGTGGAGCTGTGCGGACGCGAAAACGAGGTCAAACTGTTGGTGGAAAGAAT |
| | GATGGCCCTGCAGACCGACATCGTGGACCTCCAGCGATCCCCTATGGGCCGGAAGCAGGGTGGCACCCTCGAT |
| | GACCTGGAAGAACAGGCTCGGGAGCTGTACAGGCGCCTGCGGGAAAAGCCGCGGGACCAGAGAACTGAAGG |
| | GGATTCCCAGGAGATGGTGCGCCTGCTGCTTCAAGCCATCCAGTCATTCGAAAAGAAGGTCCGCGTGATCTAC |
| | ACCCAACTGAGCAAGACTGTGGTGTGCAAGCAGAAGGCCCTCGAACTGCTGCCGAAGGTGGAGGAGGTCGTG |
| | TCCCTGATGAACGAGGACGAAAAGACGGTCGTGAGACTCCAGGAAAAGAGACAGAAGGAACTGTGGAACCTT |
| | CTCAAGATTGCCTGCTCCAAAGTGCGCGGACCTGTGGCTGGAGCTCCCGACGCCATGAACGCCGCTAGACTCG |
| | CGCAGCCTGGACAGCTCATGGCCCAGCCCGCAACTGCAGCTAACGCCCTGCCCGAACCAGCGAAGAAGGCGG |
| | AGGAGCTTGTGGCGGAAGCCCACAACCTGTGCACCCTGCTCGAAAACGCCATCCAGGACACTGTGCGGGAACA |
| | AGACCAATCCTTCACCGCCCTGGATTGGTCATGGCTGCAGACTGAGGAAGAGGAGCACTCCTGTCTGGAGCAA |
| | GCCTCG |
| | Human constitutively active IKK beta (PEST mutation) P.4015 without epitope tag |
| 1398 | ATGAGCGCCGAGGTGATCCACCAGGTGGAGGAGGCCCTGGACACCGACGAGAAGGAGATGCTGCTGTTCCTG |
| | TGCAGAGACGTGGCCATCGACGTGGTGCCCCCCAACGTGAGAGACCTGCTGGACATCCTGAGAGAGAGAGGC |
| | AAGCTGAGCGTGGGCGACCTGGCCGAGCTGCTGTACAGAGTGAGAAGATTCGACCTGCTGAAGAGAATCCTG |
| | AAGATGGACAGAAAGGCCGTGGAGACCCACCTGCTGAGAAACCCCCACCTGGTGAGCGACTACAGAGTGCTG |
| | ATGGCCGAGATCGGCGAGGACCTGGACAAGAGCGACGTGAGCAGCCTGATCTTCCTGATGAAGGACTACATG |
| | GGCAGAGGCAAGATCAGCAAGGAGAAGAGCTTCCTGGACCTGGTGGTGGAGCTGGAGAAGCTGAACCTGGT |
| | GGCCCCCGACCAGCTGGACCTGCTGGAGAAGTGCCTGAAGAACATCCACAGAATCGACCTGAAGACCAAGATC |
| | CAGAAGTACAAGCAGAGCGTGCAGGGCGCCGGCACCAGCTACAGAAACGTGCTGCAGGCCGCCATCCAGAAG |
| | AGCCTGAAGGACCCCAGCAACAACTTCAGACTGCACAACGGCAGAAGCAAGGAGCAGAGACTGAAGGAGCAG |
| | CTGGGCGCCCAGCAGGAGCCCGTGAAGAAGAGCATCCAGGAGAGCGAGGCCTTCCTGCCCCAGAGCATCCCC |
| | GAGGAGAGATACAAGATGAAGAGCAAGCCCCTGGGCATCTGCCTGATCATCGACTGCATCGGCAACGAGACC |
| | GAGCTGCTGAGAGACACCTTCACCAGCCTGGGCTACGAGGTGCAGAAGTTCCTGCACCTGAGCATGCACGGCA |
| | TCAGCCAGATCCTGGGCCAGTTCGCCTGCATGCCCGAGCACAGAGACTACGACAGCTTCGTGTGCGTGCTGGT |
| | GAGCAGAGGCGGCAGCCAGAGCGTGTACGGCGTGGACCAGACCCACAGCGGCCTGCCCCTGCACCACATCAG |
| | AAGAATGTTCATGGGCGACAGCTGCCCCTACCTGGCCGGCAAGCCCAAGATGTTCTTCATCCAGAACTACGTGG |
| | TGAGCGAGGGCCAGCTGGAGGACAGCAGCCTGCTGGAGGTGGACGGCCCCGCCATGAAGAACGTGGAGTTC |
| | AAGGCCCAGAAGAGAGGCCTGTGCACCGTGCACAGAGAGGCCGACTTCTTCTGGAGCCTGTGCACCGCCGAC |
| | ATGAGCCTGCTGGAGCAGAGCCACAGCAGCCCCAGCCTGTACCTGCAGTGCCTGAGCCAGAAGCTGAGACAG |
| | GAGAGAAAGAGACCCCTGCTGGACCTGCACATCGAGCTGAACGGCTACATGTACGACTGGAACAGCAGAGTG |
| | AGCGCCAAGGAGAAGTACTACGTGTGGCTGCAGCACACCCTGAGAAAGAAGCTGATCCTGAGCTACACC |
| | (hu-cFLIP-L; P1006 without epitope tag) |
| 1399 | ATGAGCGCCGAGGTGATCCACCAGGTGGAGGAGGCCCTGGACACCGACGAGAAGGAGATGCTGCTGTTCCTG |
| | TGCAGAGACGTGGCCATCGACGTGGTGCCCCCCAACGTGAGAGACCTGCTGGACATCCTGAGAGAGAGAGGC |
| | AAGCTGAGCGTGGGCGACCTGGCCGAGCTGCTGTACAGAGTGAGAAGATTCGACCTGCTGAAGAGAATCCTG |
| | AAGATGGACAGAAAGGCCGTGGAGACCCACCTGCTGAGAAACCCCCACCTGGTGAGCGACTACAGAGTGCTG |
| | ATGGCCGAGATCGGCGAGGACCTGGACAAGAGCGACGTGAGCAGCCTGATCTTCCTGATGAAGGACTACATG |
| | GGCAGAGGCAAGATCAGCAAGGAGAAGAGCTTCCTGGACCTGGTGGTGGAGCTGGAGAAGCTGAACCTGGT |
| | GGCCCCCGACCAGCTGGACCTGCTGGAGAAGTGCCTGAAGAACATCCACAGAATCGACCTGAAGACCAAGATC |
| | CAGAAGTACAAGCAGAGCGTGCAGGGCGCCGGCACCAGCTACAGAAACGTGCTGCAGGCCGCCATCCAGAAG |
| | AGCCTGAAGGACCCCAGCAACAACTTCAGACTGCACAACGGCAGAAGCAAGGAGCAGAGACTGAAGGAGCAG |
| | CTGGGCGCCCAGCAGGAGCCCGTGAAGAAGAGC |
| | (hu-cFLIP-S(1-227); P1007 without epitope tag) |
| 1400 | ATGAGCGCCGAGGTGATCCACCAGGTGGAGGAGGCCCTGGACACCGACGAGAAGGAGATGCTGCTGTTCCTG |
| | TGCAGAGACGTGGCCATCGACGTGGTGCCCCCCAACGTGAGAGACCTGCTGGACATCCTGAGAGAGAGAGGC |
| | AAGCTGAGCGTGGGCGACCTGGCCGAGCTGCTGTACAGAGTGAGAAGATTCGACCTGCTGAAGAGAATCCTG |
| | AAGATGGACAGAAAGGCCGTGGAGACCCACCTGCTGAGAAACCCCCACCTGGTGAGCGACTACAGAGTGCTG |
| | ATGGCCGAGATCGGCGAGGACCTGGACAAGAGCGACGTGAGCAGCCTGATCTTCCTGATGAAGGACTACATG |
| | GGCAGAGGCAAGATCAGCAAGGAGAAGAGCTTCCTGGACCTGGTGGTGGAGCTGGAGAAGCTGAACCTGGT |
| | GGCCCCCGACCAGCTGGACCTGCTGGAGAAGTGCCTGAAGAACATCCACAGAATCGACCTGAAGACCAAGATC |
| | CAGAAGTACAAGCAGAGCGTGCAGGGCGCCGGCACCAGCTACAGAAACGTGCTGCAGGCCGCCATCCAGAAG |
| | AGCCTGAAGGAC |
| | (hu-cFLIP-p22(1-198); P1008 without epitope tag)-nucleotide |
| 1401 | ATGAGCGCCGAGGTGATCCACCAGGTGGAGGAGGCCCTGGACACCGACGAGAAGGAGATGCTGCTGTTCCTG |
| | TGCAGAGACGTGGCCATCGACGTGGTGCCCCCCAACGTGAGAGACCTGCTGGACATCCTGAGAGAGAGAGGC |
| | AAGCTGAGCGTGGGCGACCTGGCCGAGCTGCTGTACAGAGTGAGAAGATTCGACCTGCTGAAGAGAATCCTG |
| | AAGATGGACAGAAAGGCCGTGGAGACCCACCTGCTGAGAAACCCCCACCTGGTGAGCGACTACAGAGTGCTG |
| | ATGGCCGAGATCGGCGAGGACCTGGACAAGAGCGACGTGAGCAGCCTGATCTTCCTGATGAAGGACTACATG |
| | GGCAGAGGCAAGATCAGCAAGGAGAAGAGCTTCCTGGACCTGGTGGTGGAGCTGGAGAAGCTGAACCTGGT |
| | GGCCCCCGACCAGCTGGACCTGCTGGAGAAGTGCCTGAAGAACATCCACAGAATCGACCTGAAGACCAAGATC |
| | CAGAAGTACAAGCAGAGCGTGCAGGGCGCCGGCACCAGCTACAGAAACGTGCTGCAGGCCGCCATCCAGAAG |
| | AGCCTGAAGGACCCCAGCAACAACTTCAGACTGCACAACGGCAGAAGCAAGGAGCAGAGACTGAAGGAGCAG |
| | CTGGGCGCCCAGCAGGAGCCCGTGAAGAAGAGCATCCAGGAGAGCGAGGCCTTCCTGCCCCAGAGCATCCCC |
| | GAGGAGAGATACAAGATGAAGAGCAAGCCCCTGGGCATCTGCCTGATCATCGACTGCATCGGCAACGAGACC |
| | GAGCTGCTGAGAGACACCTTCACCAGCCTGGGCTACGAGGTGCAGAAGTTCCTGCACCTGAGCATGCACGGCA |
| | TCAGCCAGATCCTGGGCCAGTTCGCCTGCATGCCCGAGCACAGAGACTACGACAGCTTCGTGTGCGTGCTGGT |
| | GAGCAGAGGCGGCAGCCAGAGCGTGTACGGCGTGGACCAGACCCACAGCGGCCTGCCCCTGCACCACATCAG |
| | AAGAATGTTCATGGGCGACAGCTGCCCCTACCTGGCCGGCAAGCCCAAGATGTTCTTCATCCAGAACTACGTGG |
| | TGAGCGAGGGCCAGCTGGAGGACAGCAGCCTGCTGGAGGTGGAC |
| | (hu-cFLIP-p43(1-376); P1009 without epitope tag)-nucleotide |
| 1402 | ATGGGCCCCGCCATGAAGAACGTGGAGTTCAAGGCCCAGAAGAGAGGCCTGTGCACCGTGCACAGAGAGGCC |
| | GACTTCTTCTGGAGCCTGTGCACCGCCGACATGAGCCTGCTGGAGCAGAGCCACAGCAGCCCCAGCCTGTACC |
| | TGCAGTGCCTGAGCCAGAAGCTGAGACAGGAGAGAAAGAGACCCCTGCTGGACCTGCACATCGAGCTGAACG |
| | GCTACATGTACGACTGGAACAGCAGAGTGAGCGCCAAGGAGAAGTACTACGTGTGGCTGCAGCACACCCTGA |
| | GAAAGAAGCTGATCCTGAGCTACACC |
| | (hu-cFLIP-p12(377-480); P1010 without epitope tag)-nucleotide |
| 1403 | ATGCAGCCCGACATGAGCCTGAACGTGATCAAGATGAAGAGCAGCGACTTCCTGGAATCGGCCGAGCTGGAC |
| | AGCGGCGGCTTCGGCAAGGTGAGCCTGTGCTTCCACAGAACTCAGGGCCTGATGATCATGAAGACCGTGTACA |
| | AGGGCCCCAATTGCATCGAGCACAACGAGGCCTTACTGGAGGAGGCCAAGATGATGAACAGACTGAGACATT |
| | CGAGAGTGGTCAAGTTACTGGGCGTGATCATCGAGGAAGGCAAGTACAGCCTGGTGATGGAGTACATGGAAA |
| | AGGGCAACCTGATGCACGTGCTGAAGGCCGAGATGAGCACCCCCCTGAGCGTGAAGGGCAGAATCATCCTGG |
| | AGATTATCGAGGGGATGTGCTACCTGCACGGCAAGGGCGTGATCCACAAGGACCTGAAGCCGGAGAACATCC |
| | TGGTGGACAACGACTTCCACATCAAGATCGCCGACCTGGGCCTGGCCAGCTTTAAGATGTGGAGCAAGCTGAA |
| | CAACGAGGAGCACAACGAGTTAAGAGAGGTGGACGGCACCGCCAAGAAGAACGGCGGCACCTTATACTACAT |
| | GGCCCCCGAGCACCTGAACGATGTGAACGCCAAGCCCACCGAGAAGAGCGACGTGTACTCCTTTGCCGTGGTC |
| | CTGTGGGCCATCTTCGCCAACAAGGAGCCCTACGAGAACGCCATTTGCGAGCAGCAGCTGATCATGTGCATTA |
| | AGAGCGGCAACAGACCCGACGTGGACGACATCACCGAGTACTGCCCCAGAGAGATTATCAGCCTGATGAAGCT |
| | GTGCTGGGAGGCCAACCCCGAGGCTAGACCCACCTTCCCTGGGATCGAGGAGAAATTCAGACCCTTCTACCTG |
| | AGCCAGCTGGAGGAGAGCGTGGAAGAGGACGTGAAGAGCCTGAAGAAAGAGTACAGCAACGAGAACGCTGT |
| | GGTGAAGCGCATGCAGAGCCTGCAGCTGGACTGCGTGGCCGTCCCCAGCAGCAGAAGCAACAGTGCCACCGA |
| | GCAGCCGGGCTCGCTGCACTCCAGCCAGGGCCTGGGCATGGGCCCCGTGGAGGAGAGCTGGTTCGCCCCCTC |
| | GCTGGAGCACCCCCAGGAGGAGAACGAACCTAGCCTGCAGAGCAAGCTGCAGGACGAGGCCAACTACCACCT |
| | GTACGGCAGCAGAATGGACAGACAGACCAAGCAGCAACCAAGACAGAACGTGGCCTACAACAGAGAGGAGG |
| | AACGAAGAAGAAGAGTGAGCCACGACCCCTTCGCCCAGCAGAGACCCTACGAGAACTTCCAGAACACCGAGG |
| | GCAAGGGCACCGCCTATAGCAGCGCCGCCAGCCACGGCAACGCAGTGCACCAGCCCAGCGGCCTGACCTCTCA |
| | GCCCCAGGTGCTGTACCAGAATAATGGCCTGTATAGCAGCCACGGCTTCGGCACCAGACCCCTGGACCCAGGC |
| | ACCGCCGGCCCTAGAGTGTGGTACAGACCCATCCCAAGCCACATGCCCAGCCTGCACAACATACCGGTGCCCG |
| | AGACAAACTACTTGGGCAACACCCCCACCATGCCCTTCAGCAGCCTGCCCCCCACAGACGAGAGCATCAAGTAC |
| | ACCATCTATAACAGCACCGGCATCCAGATCGGCGCCTACAACTATATGGAGATCGGCGGTACCAGCAGCAGCG |
| | GCGGCATCAAGAAGGAGATAGAGGCAATCAAGAAGGAGCAGGAGGCCATCAAGAAGAAGATCGAAGCCATC |
| | GAGAAGGAGATTGAGGCC |
| | (huRIPK1(1-555).IZ.TM; TH1021 without epitope tag)-nucleotide |
| 1404 | ATGCAGCCCGACATGAGCCTGAATGTGATCAAGATGAAGAGCAGCGACTTCCTGGAGAGCGCCGAGCTGGAT |
| | AGCGGCGGATTCGGCAAGGTGAGCCTGTGCTTCCACAGAACCCAAGGCCTGATGATCATGAAGACCGTGTACA |
| | AGGGACCCAACTGCATCGAGCACAACGAAGCCCTGTTAGAGGAAGCCAAGATGATGAATAGACTGCGTCACTC |
| | TAGGGTGGTTAAACTGCTGGGCGTGATCATCGAGGAGGGCAAGTACAGCCTGGTGATGGAGTACATGGAGAA |
| | GGGCAACCTTATGCACGTGCTGAAGGCCGAGATGTCCACCCCCCTGAGCGTGAAGGGCAGAATCATCCTGGAG |
| | ATCATCGAGGGAATGTGTTATCTGCATGGCAAGGGCGTGATCCACAAAGACCTGAAGCCCGAGAACATCCTGG |
| | TGGACAACGATTTCCACATCAAGATCGCCGACCTGGGCCTGGCCAGCTTCAAGATGTGGAGCAAGCTGAACAA |
| | CGAGGAGCACAACGAACTGAGAGAGGTGGATGGCACCGCCAAGAAAAACGGCGGCACCCTGTATTACATGGC |
| | CCCCGAGCACCTGAACGACGTGAACGCCAAGCCCACCGAGAAGAGCGACGTTTACAGCTTTGCCGTGGTGCTG |
| | TGGGCCATCTTCGCCAACAAGGAGCCCTACGAGAACGCCATCTGCGAGCAGCAGCTGATCATGTGCATCAAGA |
| | GCGGCAACAGACCCGACGTGGACGACATCACCGAGTACTGCCCCCGTGAGATCATTAGCCTGATGAAGCTGTG |
| | CTGGGAGGCCAACCCCGAGGCCAGACCCACCTTCCCCGGCATTGAGGAGAAGTTCAGACCCTTCTACCTGAGC |
| | CAGTTAGAGGAAAGCGTGGAGGAGGACGTGAAAAGCCTGAAGAAAGAGTACTCTAACGAGAACGCCGTGGT |
| | GAAACGCATGCAGAGCCTGCAGCTGGATTGCGTGGCCGTGCCCAGCTCCAGAAGCAACAGCGCCACCGAACA |
| | ACCTGGCAGCCTGCACAGCTCCCAGGGCCTGGGCATGGGCCCCGTGGAGGAGAGCTGGTTCGCCCCCTCCCTG |
| | GAGCATCCGCAGGAGGAGAACGAGCCCTCTCTGCAGTCCAAGCTGCAAGACGAGGCCAACTACCACCTGTACG |
| | GCAGCAGAATGGACAGACAGACCAAGCAGCAACCCAGACAAAATGTGGCCTACAATAGAGAGGAGGAGAGA |
| | AGAAGAAGAGTGAGCCACGACCCTTTCGCCCAGCAGAGACCCTACGAGAACTTCCAGAATACCGAGGGCAAG |
| | GGTACCGCCTACAGCTCAGCGGCCTCGCACGGCAACGCCGTGCACCAGCCCAGCGGCCTGACCAGCCAGCCCC |
| | AGGTGCTGTACCAAAACAACGGCCTGTATAGCTCCCACGGCTTTGGCACCAGACCCCTGGACCCCGGCACCGCC |
| | GGCCCCAGAGTCTGGTATAGACCCATCCCCAGCCATATGCCTAGCCTGCACAACATCCCCGTGCCCGAGACCAA |
| | CTACCTGGGCAATACCCCCACCATGCCGTTCAGCAGCTTACCCCCCACCGACGAGAGCATCAAGTACACCATCT |
| | ACAACAGCACCGGCATCCAGATCGGCGCCTACAACTACATGGAAATCGGCGGAACCAGCAGCAGCGGCAGCG |
| | ACGGCAGCGGCTCCGGAAGCGGAAGCATAACCATCAGGGCCGCCTTCCTGGAGAAGGAAAATACCGCGCTGA |
| | GAACAGAGATTGCCGAGTTAGAAAAGGAGGTGGGCAGATGCGAGAACATAGTGAGCAAGTACGAGACCAGA |
| | TACGGCCCCCTG |
| | (huRIPK1(1-555).EE.DM; TH1022 without epitope tag)-nucleotide |
| 1405 | ATGCAACCCGACATGAGCTTGAACGTGATCAAGATGAAGAGCAGCGATTTCCTGGAGAGCGCCGAGCTGGAC |
| | AGCGGCGGCTTCGGCAAGGTGAGCCTGTGTTTCCACAGAACCCAGGGCCTGATGATCATGAAGACAGTGTACA |
| | AGGGCCCCAACTGCATCGAGCACAACGAGGCCCTGCTGGAGGAGGCTAAGATGATGAACAGACTGAGACACA |
| | GCAGAGTCGTGAAGCTGCTGGGCGTGATCATCGAAGAGGGCAAGTACAGCCTGGTGATGGAGTACATGGAGA |
| | AAGGCAACCTTATGCACGTGCTCAAGGCCGAGATGAGCACCCCTCTGAGCGTGAAGGGAAGAATCATCCTGGA |
| | GATCATCGAGGGCATGTGCTACCTGCACGGCAAGGGCGTCATCCATAAGGACCTGAAGCCCGAGAATATCCTT |
| | GTGGACAACGACTTCCATATCAAGATCGCCGACCTCGGCCTGGCCAGCTTCAAGATGTGGAGCAAGCTGAACA |
| | ACGAGGAGCACAACGAGCTGAGAGAGGTAGACGGCACCGCCAAGAAAAATGGCGGCACCCTGTACTACATGG |
| | CTCCCGAGCACCTGAATGACGTGAACGCCAAGCCTACCGAAAAGAGCGACGTGTATAGCTTCGCCGTGGTGCT |
| | CTGGGCCATCTTCGCCAACAAGGAGCCTTATGAGAATGCAATCTGCGAGCAGCAGCTGATCATGTGCATCAAG |
| | AGCGGCAACAGACCCGACGTGGACGACATCACCGAATACTGCCCCAGAGAGATCATCAGCCTGATGAAGCTGT |
| | GCTGGGAGGCCAACCCCGAGGCCAGACCCACCTTCCCCGGCATTGAGGAGAAGTTCAGACCCTTCTACCTGAG |
| | CCAGTTGGAAGAGAGCGTGGAGGAGGACGTCAAAAGCCTGAAGAAGGAGTACAGCAACGAGAACGCCGTCG |
| | TGAAGAGAATGCAGAGCCTGCAGCTGGACTGCGTGGCCGTGCCTAGCAGCAGAAGCAACAGCGCCACCGAGC |
| | AGCCCGGCAGCCTGCACAGCAGCCAGGGCCTTGGAATGGGCCCCGTGGAGGAAAGCTGGTTCGCCCCCAGCC |
| | TTGAGCATCCGCAGGAGGAGAACGAGCCCAGCCTGCAGAGCAAGCTGCAGGACGAAGCCAACTATCACCTGT |
| | ACGGCAGCAGAATGGACCGACAGACCAAGCAGCAGCCCAGACAGAACGTGGCCTATAACCGAGAGGAGGAG |
| | AGAAGAAGAAGGGTGAGCCACGACCCCTTCGCCCAACAGAGACCCTACGAGAACTTCCAGAACACCGAGGGC |
| | AAGGGCACCGCTTACAGTAGCGCCGCAAGCCACGGCAACGCCGTGCACCAACCTAGCGGACTGACCAGCCAG |
| | CCCCAGGTGCTGTACCAAAACAACGGTCTGTACAGCTCACACGGCTTCGGGACCAGACCCTTAGATCCCGGAAC |
| | CGCCGGCCCCAGAGTATGGTATAGACCCATCCCCAGCCACATGCCCAGCTTGCACAACATCCCCGTGCCCGAGA |
| | CCAACTACCTGGGCAACACCCCCACCATGCCCTTCAGCAGCCTGCCCCCCACCGACGAGAGCATCAAATATACC |
| | ATCTACAACAGCACCGGAATCCAGATCGGGGCCTACAATTACATGGAGATCGGAGGCACCAGCAGCAGCGGC |
| | AGCGACGGTAGCGGAAGCGGCAGCGGCAGCCTCGAGATCAGAGCCGCCTTCCTGGAGAAGGAGAACACCGC |
| | CCTGAGAACCAGAGCCGCCGAACTGAGAAAGAGAGTGGGCAGATGCAGAAACATCGTGAGCAAGTACGAGA |
| | CCAGATACGGCCCCCTG |
| | (huRIPK1(1-555).RR.DM; TH1023 without epitope tag)-nucleotide |
| 1406 | ATGCAGCCTGACATGAGCCTGGACAATATCAAGATGGCCAGCAGCGACCTGCTCGAGAAGACCGACCTGGACA |
| | GTGGCGGCTTCGGAAAAGTGAGCCTGTGCTACCACAGGTCTCACGGGTTCGTGATCCTGAAGAAGGTGTACAC |
| | CGGCCCCAACAGAGCCGAGTATAATGAGGTGCTGCTGGAGGAGGGCAAGATGATGCACAGACTGAGACATAG |
| | CAGAGTGGTGAAGCTGCTGGGCATCATCATCGAGGAGGGAAACTACAGCCTGGTTATGGAGTACATGGAGAA |
| | GGGCAACCTAATGCACGTGTTGAAGACCCAGATAGACGTGCCACTGAGCTTAAAGGGCAGAATCATCGTGGA |
| | GGCTATCGAGGGCATGTGCTACCTGCACGACAAGGGCGTGATCCACAAAGACCTGAAGCCCGAGAACATACTC |
| | GTGGATAGAGATTTCCACATCAAGATCGCCGACCTGGGCGTGGCCAGCTTCAAGACTTGGAGCAAGCTGACAA |
| | AGGAGAAGGACAACAAGCAGAAGGAGGTGAGCAGCACCACCAAGAAAAACAACGGCGGCACCCTGTACTAC |
| | ATGGCCCCTGAGCACCTGAACGACATCAACGCCAAGCCCACCGAGAAGAGCGACGTGTATAGCTTCGGCATCG |
| | TGCTGTGGGCCATCTTTGCTAAGAAAGAGCCCTACGAGAACGTGATCTGCACCGAGCAGTTCGTCATCTGCATC |
| | AAGAGCGGCAACAGACCCAATGTGGAGGAGATCCTGGAATACTGCCCCAGAGAGATCATCAGCCTCATGGAG |
| | AGATGCTGGCAGGCCATCCCTGAGGACAGACCCACCTTCCTGGGCATTGAGGAGGAGTTCAGACCCTTCTACC |
| | TGAGCCACTTCGAGGAGTACGTGGAGGAGGACGTGGCCAGTCTGAAAAAGGAGTATCCAGACCAGAGCCCCG |
| | TGCTGCAGAGAATGTTCAGCCTGCAGCACGACTGTGTGCCCCTGCCCCCCAGCAGAAGCAACAGCGAGCAGCC |
| | GGGCAGCCTGCACAGCAGCCAGGGCTTACAAATGGGACCCGTGGAGGAGAGCTGGTTCAGCAGTAGCCCCGA |
| | GTACCCCCAGGACGAGAACGACAGGTCGGTCCAGGCCAAGCTCCAGGAAGAGGCCAGCTACCACGCCTTCGG |
| | CATCTTCGCCGAGAAGCAAACCAAGCCCCAGCCCAGACAAAACGAAGCCTACAACAGAGAGGAAGAGAGAAA |
| | GAGACGCGTAAGCCACGACCCCTTTGCCCAACAGAGAGCCAGAGAAAACATCAAGAGCGCCGGCGCCCGGGG |
| | CCACTCGGATCCGAGCACCACTAGCAGAGGCATCGCTGTGCAGCAACTCAGCTGGCCCGCCACCCAGACCGTG |
| | TGGAACAACGGCCTGTACAACCAGCACGGCTTCGGCACCACCGGCACCGGCGTTTGGTACCCCCCCAACCTGTC |
| | GCAGATGTACAGCACCTACAAAACCCCCGTGCCCGAGACCAACATCCCCGGCAGCACCCCCACCATGCCCTATT |
| | TCAGCGGCCCCGTGGCCGACGACCTGATCAAGTACACCATCTTCAACAGCAGCGGCATCCAGATCGGCAACCA |
| | CAATTACATGGACGTGGGCCTGAACAGCCAGCCACCCAACAACACCTGCAAGGAAGAAAGCACCAGCGGCGG |
| | CATCAAGAAGGAAATCGAGGCCATCAAGAAGGAGCAGGAAGCCATAAAGAAGAAAATCGAGGCCATCGAGA |
| | AGGAGATCGAGGCC |
| | (msRIPK1(1-555).IZ.TM; TH1024 without epitope tag)-nucleotide |
| 1407 | ATGCAGCCCGACATGAGCCTGGACAACATTAAGATGGCCAGTAGCGACCTGCTGGAGAAGACCGACCTGGAT |
| | AGCGGGGGCTTCGGCAAGGTGAGCCTGTGCTACCACAGAAGCCACGGATTCGTGATCCTGAAGAAGGTGTAC |
| | ACCGGCCCCAACAGAGCCGAGTACAACGAGGTGCTGCTGGAGGAGGGCAAGATGATGCATAGACTGAGACAC |
| | AGCAGAGTGGTGAAACTGCTGGGGATCATCATCGAAGAGGGCAACTATAGCCTGGTGATGGAATACATGGAG |
| | AAGGGCAACCTGATGCACGTGCTGAAGACCCAGATCGACGTGCCCCTGAGCCTGAAGGGCAGAATCATCGTG |
| | GAGGCCATCGAGGGTATGTGCTACCTGCACGATAAGGGCGTGATCCACAAGGACCTGAAACCTGAAAACATCT |
| | TAGTGGACAGAGACTTCCACATCAAGATCGCCGACCTGGGAGTGGCTAGCTTCAAGACCTGGAGCAAACTGAC |
| | CAAGGAGAAGGATAACAAGCAGAAGGAAGTGAGCAGCACCACCAAGAAAAACAACGGAGGCACCCTGTACT |
| | ACATGGCCCCCGAGCATCTGAACGACATCAACGCCAAGCCCACCGAGAAGAGCGACGTGTACTCCTTCGGCAT |
| | CGTCTTATGGGCCATCTTCGCCAAGAAGGAGCCCTACGAGAACGTGATCTGCACCGAACAGTTTGTGATCTGCA |
| | TCAAGAGCGGCAATAGACCCAACGTGGAGGAGATCCTGGAGTACTGCCCCAGAGAGATCATCAGCCTGATGG |
| | AGAGGTGCTGGCAGGCTATCCCCGAGGACAGACCCACCTTTCTGGGCATCGAGGAAGAGTTCAGACCCTTCTA |
| | TCTGAGCCACTTCGAGGAGTATGTTGAGGAGGACGTGGCCAGCCTGAAGAAGGAGTACCCCGACCAGAGCCC |
| | CGTGCTGCAGAGAATGTTCAGCCTGCAACACGATTGCGTGCCGCTGCCCCCCAGCAGATCGAATAGCGAGCAG |
| | CCAGGCAGCCTACACAGCAGTCAGGGCCTGCAGATGGGCCCCGTGGAGGAAAGCTGGTTCAGCAGCAGCCCC |
| | GAGTACCCCCAGGACGAGAATGACAGAAGCGTGCAAGCAAAGCTGCAAGAGGAGGCCAGCTACCACGCCTTC |
| | GGCATCTTCGCCGAGAAACAGACTAAGCCCCAGCCCAGACAGAACGAGGCCTACAACAGAGAGGAGGAGAGA |
| | AAAAGACGAGTGAGCCACGACCCCTTCGCCCAGCAGAGAGCCAGAGAGAATATCAAGAGCGCCGGCGCCAGA |
| | GGCCACAGCGACCCCAGCACCACCAGCAGAGGAATCGCCGTGCAGCAGCTGAGCTGGCCCGCCACCCAGACC |
| | GTGTGGAACAACGGCCTGTACAACCAGCACGGCTTTGGCACCACCGGCACCGGCGTGTGGTATCCCCCCAACC |
| | TGAGCCAGATGTACAGCACCTATAAAACCCCTGTGCCGGAGACCAATATCCCCGGCAGCACCCCTACCATGCCC |
| | TACTTCAGCGGCCCCGTGGCCGACGACCTGATCAAGTACACGATCTTCAACAGCAGCGGCATCCAGATAGGCA |
| | ACCACAACTACATGGACGTGGGCCTGAACAGCCAACCCCCCAATAACACCTGCAAGGAGGAGTCCACCAGCGG |
| | CAGCGACGGCAGCGGCAGCGGCAGCGGCAGCATAACCATCAGAGCTGCTTTCCTGGAGAAGGAGAACACCGC |
| | TCTGAGAACCGAGATCGCCGAGCTGGAGAAGGAGGTCGGCAGATGCGAGAATATCGTGAGCAAGTACGAGA |
| | CCAGATACGGACCCCTG |
| | (msRIPK1(1-555).EE.DM; TH1025 without epitope tag)-nucleotide |
| 1408 | ATGCAGCCTGATATGAGCCTGGACAACATCAAGATGGCCAGCAGCGACTTGCTGGAGAAGACCGATCTGGACT |
| | CCGGCGGCTTTGGCAAGGTGAGCCTGTGTTACCACAGAAGCCACGGCTTCGTGATCCTGAAAAAGGTGTACAC |
| | CGGCCCCAATAGAGCAGAGTACAACGAGGTGCTGCTGGAGGAGGGCAAGATGATGCACAGACTGAGGCATA |
| | GCAGAGTGGTGAAACTGCTGGGCATCATCATTGAGGAGGGCAACTACAGCCTGGTGATGGAGTACATGGAGA |
| | AGGGCAACCTGATGCATGTGCTGAAGACCCAAATCGACGTGCCCCTGTCGCTGAAGGGCAGAATCATCGTGGA |
| | GGCCATCGAGGGGATGTGCTACCTGCACGACAAGGGCGTGATCCACAAGGACCTGAAGCCCGAGAACATCCT |
| | GGTGGATAGAGACTTCCACATCAAGATCGCCGACCTGGGCGTTGCCAGCTTCAAGACCTGGTCTAAACTGACC |
| | AAGGAGAAAGACAACAAGCAGAAGGAGGTGAGCAGCACCACCAAGAAGAACAACGGCGGAACACTGTACTA |
| | TATGGCCCCTGAGCACCTGAACGACATCAACGCCAAGCCCACCGAGAAAAGCGATGTTTACAGCTTCGGCATC |
| | GTGCTGTGGGCCATCTTCGCCAAGAAGGAGCCCTACGAGAACGTGATCTGCACCGAGCAGTTCGTGATCTGCA |
| | TCAAGAGCGGCAACAGACCCAACGTGGAGGAAATCCTGGAGTACTGCCCCAGAGAGATCATCAGCCTGATGG |
| | AGAGATGCTGGCAGGCCATCCCCGAGGACCGTCCCACGTTCCTGGGCATCGAAGAGGAGTTCCGGCCCTTCTA |
| | CCTGAGCCATTTCGAGGAGTATGTGGAGGAGGACGTGGCCAGCCTGAAGAAGGAGTACCCCGACCAGAGCCC |
| | AGTGCTGCAGAGAATGTTCAGCCTTCAACACGACTGCGTGCCCCTGCCTCCCTCAAGAAGCAACAGCGAGCAG |
| | CCCGGCAGCTTGCACAGCAGCCAGGGCCTGCAGATGGGCCCCGTGGAGGAGAGCTGGTTTAGCAGCAGCCCC |
| | GAGTACCCCCAGGACGAGAATGACAGAAGCGTGCAAGCCAAGTTACAGGAGGAGGCCAGCTACCACGCCTTT |
| | GGAATCTTCGCCGAGAAGCAGACCAAGCCCCAGCCCAGACAGAACGAGGCCTACAACAGAGAGGAGGAGAG |
| | AAAAAGAAGAGTGAGCCACGACCCCTTCGCCCAGCAGAGAGCCAGAGAGAACATTAAGAGCGCCGGCGCGAG |
| | AGGCCACAGCGACCCCAGCACCACAAGCAGAGGCATCGCCGTGCAGCAATTGAGCTGGCCCGCCACCCAGACC |
| | GTGTGGAACAACGGCCTGTATAACCAGCACGGCTTCGGAACCACCGGCACCGGCGTGTGGTACCCCCCCAATC |
| | TGAGCCAGATGTACAGCACTTACAAGACCCCCGTGCCCGAAACCAACATCCCCGGCAGCACCCCCACCATGCCC |
| | TACTTCAGCGGCCCCGTGGCCGACGACCTCATCAAGTACACAATATTTAACAGCAGCGGCATCCAGATCGGCAA |
| | CCACAACTACATGGACGTGGGCCTGAACAGCCAGCCCCCGAACAATACCTGCAAGGAGGAGAGCACAAGCGG |
| | CTCTGACGGCAGCGGCAGCGGCAGCGGCTCACTGGAGATCAGAGCTGCCTTCCTGGAAAAGGAGAACACCGC |
| | TCTGAGAACCAGAGCCGCCGAGCTGCGAAAGAGAGTAGGCAGATGCAGAAACATCGTGAGCAAGTACGAGAC |
| | CAGATACGGTCCCCTG |
| | (msRIPK1(1-555).RR.DM; TH1026 without epitope tag)-nucleotide |
| 1409 | ATGAGCGCCGGCGACCCCAGAGTGGGCAGCGGCAGCCTGGACAGCTTCATGTTCAGCATCCCCCTGGTGGCCC |
| | TGAACGTGGGCGTGAGAAGAAGACTGAGCCTGTTCCTGAACCCCAGAACCCCCGTGGCCGCCGACTGGACCCT |
| | GCTGGCCGAGGAGATGGGCTTCGAGTACCTGGAGATCAGAGAGCTGGAGACCAGACCCGACCCCACCAGAAG |
| | CCTGCTGGACGCCTGGCAGGGCAGAAGCGGCGCCAGCGTGGGCAGACTGCTGGAGCTGCTGGCCCTGCTGGA |
| | CAGAGAGGACATCCTGAAGGAGCTGAAGAGCAGAATCGAGGAGGACTGCCAGAAGTACCTGGGCAAGCAGC |
| | AGAACCAGGAGAGCGAGAAGCCCCTGCAGGTGGCCAGAGTGGAGAGCAGCGTGCCCCAGACCAAGGAGCTG |
| | GGCGGCATCACCACCCTGGACGACCCCCTGGGCCAGACCCCCGAGCTGTTCGACGCCTTCATCTGCTACTGCCC |
| | CAACGACATCGAGTTCGTGCAGGAGATGATCAGACAGCTGGAGCAGACCGACTACAGACTGAAGCTGTGCGT |
| | GAGCGACAGAGACGTGCTGCCCGGCACCTGCGTGTGGAGCATCGCCAGCGAGCTGATCGAGAAGAGATGCAG |
| | AAGAATGGTGGTGGTGGTGAGCGACGACTACCTGCAGAGCAAGGAGTGCGACTTCCAGACCAAGTTCGCCCT |
| | GAGCCTGAGCCCCGGCGTGCAGCAGAAGAGACCCATCCCCATCAAGTACAAGGCCATGAAGAAGGACTTCCCC |
| | AGCATCCTGAGATTCATCACCATCTGCGACTACACCAACCCCTGCACCAAGAGCTGGTTCTGGACCAGACTGGC |
| | CAAGGCCCTGAGCCTGCCC |
| | (human myd88(L265P); P4027 without epitope tag)-nucleotide |
| 1410 | ATGGGCGTGGGCAAGAGCAAGCTGGACAAGTGCCCCCTGAGCTGGCACAAGAAGGACAGCGTGGACGCCGA |
| | CCAGGACGGCCACGAGAGCGACAGCAAGAACAGCGAGGAGGCCTGCCTGAGAGGCTTCGTGGAGCAGAGCA |
| | GCGGCAGCGAGCCCCCCACCGGCGAGCAGGACCAGCCCGAGGCCAAGGGCGCCGGCCCCGAGGAGCAGGAC |
| | GAGGAGGAGTTCCTGAAGTTCGTGATCCTGCACGCCGAGGACGACACCGACGAGGCCCTGAGAGTGCAGGAC |
| | CTGCTGCAGAACGACTTCGGCATCAGACCCGGCATCGTGTTCGCCGAGATGCCCTGCGGCAGACTGCACCTGC |
| | AGAACCTGGACGACGCCGTGAACGGCAGCGCCTGGACCATCCTGCTGCTGACCGAGAACTTCCTGAGAGACAC |
| | CTGGTGCAACTTCCAGTTCTACACCAGCCTGATGAACAGCGTGAGCAGACAGCACAAGTACAACAGCGTGATC |
| | CCCATGAGACCCCTGAACAGCCCCCTGCCCAGAGAGAGAACCCCCCTGGCCCTGCAGACCATCAACGCCCTGG |
| | AGGAGGAGAGCCAGGGCTTCAGCACCCAGGTGGAGAGAATCTTCAGAGAGAGCGTGTTCGAGAGACAGCAG |
| | AGCATCTGGAAGGAGACCAGAAGCGTGAGCCAGAAGCAGTTCATCGCC |
| | (Mouse TRAM (TICAM2); P4033 without epitope tag)-nucleotide |
| 1411 | ATGAGCACCGCCAGCGCCGCCAGCTCAAGCTCCTCCTCTAGCGCCGGCGAGATGATCGAGGCCCCCAGCCAGG |
| | TGCTGAACTTCGAGGAGATCGACTACAAGGAAATCGAGGTGGAGGAGGTGGTGGGCAGAGGCGCCTTCGGC |
| | GTGGTGTGCAAGGCCAAGTGGAGAGCCAAGGACGTGGCCATCAAGCAGATCGAGAGCGAGTCCGAGAGAAA |
| | GGCCTTCATCGTGGAGCTGAGACAGCTGAGCAGAGTGAACCACCCCAACATCGTGAAGCTGTACGGCGCCTGC |
| | CTGAACCCCGTGTGCCTGGTGATGGAGTACGCCGAGGGCGGCAGCCTGTACAACGTGCTGCACGGCGCCGAG |
| | CCCCTGCCCTACTACACCGCCGCCCACGCCATGAGCTGGTGCCTGCAGTGCAGCCAGGGCGTGGCCTACCTGCA |
| | CAGCATGCAGCCCAAGGCCCTGATCCACCGCGATCTGAAGCCCCCCAACCTGCTGCTGGTGGCCGGCGGCACC |
| | GTGCTGAAGATCTGCGACTTCGGCACCGCCTGCGACATCCAGACCCACATGACCAACAACAAGGGATCAGCTG |
| | CGTGGATGGCCCCCGAGGTGTTCGAGGGCAGCAACTACAGCGAGAAGTGCGACGTGTTCAGCTGGGGCATCA |
| | TCCTGTGGGAGGTGATCACCAGAAGAAAGCCCTTCGACGAGATCGGCGGCCCCGCCTTCAGAATCATGTGGGC |
| | CGTGCACAACGGCACCAGACCGCCGCTGATCAAGAACCTGCCCAAGCCCATCGAGTCCCTGATGACCAGATGC |
| | TGGAGCAAGGACCCGAGCCAGAGGCCCAGCATGGAAGAGATCGTTAAGATCATGACCCACCTGATGAGATAC |
| | TTCCCGGGCGCCGATGAACCGCTGCAGTACCCCTGCCAGGAGTTCGGCGGAGGCGGCGGCCAGAGCCCCACC |
| | CTGACCCTGCAGAGCACCAACACCCACACCCAGAGCAGCAGCAGTAGCAGCGACGGCGGCCTGTTCAGAAGC |
| | AGACCCGCCCACAGCCTGCCCCCCGGCGAGGACGGCAGAGTGGAGCCCTACGTGGACTTCGCCGAGTTCTACA |
| | GACTGTGGAGCGTGGACCACGGCGAGCAGAGCGTGGTGACCGCCCCC |
| | (human TAK1-TAB1; P4031 without epitope tag)-nucleotide |
| 1412 | ATGGAGAACCTGAAGCACATCATCACCCTGGGCCAGGTGATCCACAAGAGATGCGAGGAGATGAAGTACTGC |
| | AAGAAGCAGTGCAGAAGACTGGGCCACAGAGTGCTGGGCCTGATCAAGCCCCTGGAGATGCTGCAGGACCAG |
| | GGCAAGAGAAGCGTGCCCAGCGAGAAGCTGACCACCGCCATGAACAGATTCAAGGCCGCCCTGGAGGAGGCC |
| | AACGGCGAGATCGAGAAGTTCAGCAACAGAAGCAACATCTGCAGATTCCTGACCGCCAGCCAGGACAAGATCC |
| | TGTTCAAGGACGTGAACAGAAAGCTGAGCGACGTGTGGAAGGAGCTGAGCCTGCTGCTGCAGGTGGAGCAGA |
| | GAATGCCCGTGAGCCCCATCAGCCAGGGCGCCAGCTGGGCCCAGGAGGACCAGCAGGACGCCGACGAGGAC |
| | AGAAGAGCCTTCCAGATGCTGAGAAGAGACAACGAGAAGATCGAGGCCAGCCTGAGAAGACTGGAGATCAAC |
| | ATGAAGGAGATCAAGGAGACCCTGAGACAGTAC |
| | (human MLKL(1-180) ORF nucleotide sequence; no epitope tag) |
| 1413 | ATGGAGCACGACCTTGAGAGAGGCCCTCCGGGCCCTAGAAGACCTCCTCGAGGTCCTCCACTTAGCAGCAGCT |
| | TGGGCCTCGCTCTCTTATTGTTGCTACTTGCCTTGTTGTTCTGGTTGTACATCGTGATGAGCGACTGGACCGGCG |
| | GCGCCCTTCTGGTGCTGTACAGCTTCGCCCTGATGCTGATCATTATCATACTGATTATCTTCATATTCAGAAGAG |
| | ATCTGCTGTGCCCTCTGGGCGCCTTATGCATTCTGCTTTTGATGATCACTCTGCTCCTCATCGCACTCTGGAACCT |
| | GCACGGCCAGGCCCTGTTCCTGGGCATCGTGCTGTTCATCTTCGGCTGCCTCCTCGTGCTTGGAATCTGGATCTA |
| | CCTGCTGGAGATGCTGTGGAGACTAGGTGCCACCATCTGGCAGCTGCTGGCCTTCTTCCTGGCATTCTTCTTAG |
| | ACCTGATTCTGCTCATTATTGCCCTATACCTGCAGCAGAACTGGTGGACCCTACTCGTTGATCTCCTGTGGCTAC |
| | TGCTGTTCCTTGCTATCCTGATTTGGATGTACTACCACGGACAAAGACCTTTCGCCGAGGACAAGACCTACAAG |
| | TACATCTGCAGAAACTTCAGCAACTTCTGCAACGTGGACGTGGTGGAGATCCTGCCTTACCTGCCTTGCCTGAC |
| | CGCCAGGGACCAGGACAGACTGAGAGCCACCTGCACCCTGAGCGGCAACAGAGACACCCTGTGGCACCTGTTC |
| | AACACCCTGCAGAGGCGCCCTGGCTGGGTGGAGTACTTCATCGCCGCCCTGAGAGGCTGCGAGTTGGTTGACC |
| | TCGCCGACGAGGTGGCCAGCGTGTACCAGAGCTACCAGCCTAGAACCAGCGACAGGCCGCCTGACCCTCTGGA |
| | GCCTCCTAGCCTGCCTGCCGAACGGCCTGGCCCACCTACCCCTGCCGCCGCCCACAGCATCCCTTACAACTCCTG |
| | TCGGGAGAAGGAGCCTAGCTACCCTATGCCTGTGCAGGAAACGCAGGCCCCAGAAAGTCCTGGCGAGAACAG |
| | CGAGCAGGCCTTGCAGACTCTGAGCCCTAGAGCCATCCCTAGAAACCCTGACGGCGGTCCTCTCGAGAGTTCCA |
| | GCGACCTGGCTGCACTCTCCCCACTGACCAGCAGCGGCCACCAGGAGCAGGACACCGAGCTGGGCAGCACCCA |
| | CACCGCCGGCGCTACCTCAAGCCTTACCCCTAGCCGGGGCCCAGTCAGCCCTAGCGTGAGCTTCCAGCCTCTGG |
| | CCAGAAGCACACCAAGAGCCAGCAGACTTCCAGGACCAACCGGCAGCGTGGTGAGCACCGGCACCAGCTTCA |
| | GTTCCTCTAGCCCAGGCTTAGCCAGCGCCGGAGCGGCCGAGGGCAAGCAGGGCGCCGAGAGCGACCAGGCCG |
| | AGCCTATCATCTGTTCCTCGGGTGCCGAGGCCCCTGCCAACAGCCTACCTAGCAAGGTGCCTACCACACTGATG |
| | CCAGTTAACACCGTGGCCCTGAAGGTTCCAGCCAACCCTGCTTCCGTTTCTACAGTGCCGTCCAAGCTGCCGAC |
| | GTCATCCAAGCCTCCGGGAGCCGTGCCATCTAACGCCCTGACCAATCCAGCTCCAAGCAAGCTCCCAATCAACA |
| | GCACCAGAGCCGGCATGGTGCCTTCAAAGGTGCCGACCTCCATGGTGCTGACCAAGGTGAGCGCCTCTACCGT |
| | GCCAACCGACGGATCTTCTCGGAACGAGGAGACACCTGCTGCTCCTACTCCAGCGGGCGCAACTGGAGGCTCC |
| | TCGGCTTGGCTGGACAGTTCTAGCGAGAATAGAGGCCTGGGTAGTGAGCTGAGTAAGCCGGGCGTGCTCGCA |
| | AGCCAGGTGGACAGCCCTTTCAGCGGCTGCTTCGAAGACCTTGCAATTTCCGCATCTACCAGTCTAGGCATGGG |
| | CCCTTGCCACGGCCCTGAGGAGAACGAGTACAAGAGCGAGGGCACCTTCGGCATCCACGTGGCCGAGAACCCT |
| | AGCATCCAGCTGCTTGAGGGCAATCCTGGACCACCAGCCGATCCTGATGGCGGACCTAGACCTCAGGCCGACA |
| | GAAAGTTCCAGGAGAGAGAGGTGCCTTGTCATAGACCTTCCCCAGGCGCTCTTTGGCTGCAGGTGGCCGTGAC |
| | CGGTGTCCTCGTCGTGACATTACTGGTGGTGCTCTACAGAAGAAGACTGCAC |
| | (CA-hMAVS ORF nucleotide sequence; no epitope tag) |
| 1414 | ATGAGCTGGTCCCCAAGCCTCACGACCCAGACCTGCGGCGCTTGGGAGATGAAGGAGAGACTGGGCACGGGG |
| | GGCTTTGGCAACGTGATCAGATGGCATAATCAGGAAACCGGAGAGCAGATTGCTATCAAGCAGTGTAGACAG |
| | GAGCTAAGCCCCCGCAATAGAGAGAGGTGGTGCCTGGAAATTCAGATTATGAGAAGACTGACCCATCCCAATG |
| | TGGTCGCCGCAAGAGACGTCCCCGAAGGCATGCAGAACCTGGCCCCCAATGACCTGCCTCTTCTGGCCATGGA |
| | ATACTGCCAGGGCGGCGACCTGCGGAAGTACCTGAATCAGTTTGAAAATTGCTGCGGCCTGAGAGAGGGCGC |
| | CATATTGACACTGCTGAGCGACATCGCCAGCGCCCTGAGATACCTGCACGAGAACAGAATAATTCACAGAGAC |
| | CTGAAGCCGGAGAATATTGTGCTGCAGCAGGGTGAACAGAGGCTCATCCATAAGATCATCGACCTGGGGTACG |
| | CCAAGGAGCTGGATCAGGGCGAGCTGTGTACCGAGTTTGTGGGGACTCTGCAATACCTGGCCCCCGAGCTCCT |
| | GGAACAGCAGAAGTACACCGTCACAGTGGATTATTGGAGCTTCGGCACGCTGGCCTTCGAGTGCATCACGGGC |
| | TTTAGGCCGTTTCTGCCCAATTGGCAGCCCGTGCAATGGCACAGCAAGGTCAGACAGAAAAGCGAGGTCGACA |
| | TCGTAGTGAGCGAAGACCTGAACGGCACTGTCAAGTTCAGTAGCTCCCTCCCCTACCCTAACAATCTGAACAGC |
| | GTGCTGGCAGAGCGGCTGGAGAAGTGGCTACAACTAATGCTGATGTGGCACCCCCGACAGCGTGGCACCGAC |
| | CCCACCTACGGGCCCAACGGATGCTTCAAGGCCCTGGACGACATTCTCAACCTGAAGCTGGTGCACATCTTGAA |
| | TATGGTGACCGGCACCATCCACACCTACCCCGTGACCGAAGACGAAAGCTTGCAGAGCCTGAAGGCCAGAATT |
| | CAACAGGACACAGGCATCCCCGAAGAGGATCAAGAGCTGCTGCAGGAAGCCGGCCTGGCTTTGATTCCCGACA |
| | AACCAGCCACCCAGTGCATTAGCGACGGCAAGCTGAACGAGGGCCACACCCTGGACATGGACCTGGTGTTCCT |
| | GTTCGACAACAGCAAGATTACCTACGAGACCCAAATCAGCCCAAGGCCCCAACCCGAGAGCGTGAGCTGCATC |
| | CTGCAAGAGCCCAAGAGGAATCTGGCCTTCTTCCAACTAAGAAAGGTGTGGGGCCAAGTGTGGCACAGCATCC |
| | AGACTCTGAAGGAAGACTGCAATAGACTGCAACAAGGACAGCGAGCCGCCATGATGAACCTGTTAAGAAACA |
| | ACAGCTGCTTATCTAAGATGAAGAACAGCATGGCCTCCATGAGCCAGCAGCTGAAAGCCAAACTGGATTTCTTC |
| | AAGACCAGCATCCAGATCGACCTGGAGAAGTACAGCGAGCAGACGGAGTTCGGGATCACCAGCGACAAGCTG |
| | CTGCTGGCTTGGAGGGAAATGGAACAGGCCGTGGAGCTGTGCGGCAGAGAGAACGAGGTTAAACTGCTGGTA |
| | GAGCGGATGATGGCCCTGCAGACCGACATTGTAGACCTCCAGAGAAGCCCTATGGGAAGAAAACAGGGCGGA |
| | ACACTGGACGACCTGGAGGAGCAGGCTAGAGAGCTGTACAGAAGACTTAGAGAGAAGCCCAGAGACCAAAG |
| | AACCGAGGGCGACAGCCAGGAGATGGTGAGACTGCTGCTACAGGCTATTCAAAGTTTCGAGAAGAAAGTGAG |
| | AGTGATCTACACCCAACTCAGCAAAACCGTGGTGTGTAAGCAGAAGGCCCTGGAGCTGCTGCCCAAGGTTGAG |
| | GAGGTTGTCAGCCTGATGAATGAGGATGAGAAGACCGTGGTGAGACTGCAAGAGAAAAGGCAGAAAGAACT |
| | GTGGAACCTTTTAAAGATTGCCTGCAGCAAGGTGAGGGGCCCTGTATCAGGATCCCCCGACTCTATGAACGCC |
| | AGCAGACTGAGCCAGCCCGGTCAACTGATGAGCCAGCCCTCTACCGCCAGCAACTCCCTGCCCGAGCCAGCCA |
| | AGAAGAGCGAGGAACTGGTGGCCGAGGCCCACAATCTGTGCACCCTACTGGAGAACGCCATTCAGGACACCG |
| | TTCGCGAGCAGGACCAGAGCTTCACCGCCCTGGACTGGAGCTGGCTGCAGACTGAGGAGGAAGAGCACAGCT |
| | GCCTGGAGCAGGCCAGC |
| | (huIKK2ca(S177E/S181E); P4005 without epitope tag)-nucleotide |
| 1415 | ATGAGCAGCGTGAAGCTCTGGCCCACCGGCGCCAGCGCCGTGCCCCTAGTGAGCCGGGAGGAGCTTAAGAAG |
| | CTCGAGTTCGTGGGCAAGGGCGGCTTCGGCGTGGTGTTCCGGGCCCACCACCGGACCTGGAACCACGACGTG |
| | GCCGTGAAGATCGTGAACAGCAAGAAGATCAGCTGGGAGGTGAAGGCCATGGTGAACCTGCGGAACGAGAA |
| | CGTGTTGCTGCTGCTGGGCGTGACCGAGGACCTGCAGTGGGACTTCGTGAGCGGCCAGGCCTTGGTTACCCGG |
| | TTCATGGAGAACGGCAGCCTGGCCGGCCTGCTGCAGCCCGAGTGCCCCCGGCCCTGGCCCCTGCTGTGCCGGC |
| | TACTGCAGGAGGTGGTGCTGGGCATGTGCTACCTGCACAGCCTGAACCCCCCACTTCTGCACCGGGACCTGAA |
| | GCCCAGCAACATCCTGCTGGACCCCGAGCTGCACGCCAAGCTGGCCGACTTCGGCCTGAGCACCTTCCAGGGC |
| | GGCAGCCAGAGCGGCTCCGGATCTGGCAGCGGAAGCCGGGACAGCGGCGGCACCCTGGCCTACCTGGACCCA |
| | GAGCTGCTGTTCGACGTGAACCTCAAGGCCAGCAAGGCCTCCGACGTGTACAGCTTCGGCATCCTGGTGTGGG |
| | CCGTGCTGGCTGGAAGGGAGGCCGAGCTGGTGGACAAGACCAGCCTGATCCGGGAGACAGTGTGCGACCGG |
| | CAGAGCCGGCCTCCTCTCACCGAACTGCCCCCCGGCAGCCCCGAGACTCCTGGCCTGGAGAAGCTGAAGGAGC |
| | TCATGATCCACTGCTGGGGCTCCCAGAGCGAGAACCGGCCCAGCTTCCAGGACTGCGAGCCCAAGACCAACGA |
| | GGTGTACAACCTGGTGAAGGACAAGGTGGACGCCGCCGTGAGCGAGGTCAAGCACTACCTGAGCCAGCACCG |
| | GAGCAGCGGCCGGAACCTGAGCGCCCGGGAGCCCAGCCAGCGGGGCACCGAGATGGACTGTCCTCGCGAGA |
| | CAATGGTGAGCAAGATGCTGGATCGGCTGCACCTGGAGGAGCCTTCAGGCCCCGTGCCCGGCAAGTGTCCTGA |
| | GAGACAGGCCCAGGACACCAGCGTGGGCCCTGCCACCCCTGCACGGACCAGCAGCGACCCCGTGGCCGGCAC |
| | CCCCCAGATCCCCCACACCCTGCCCTTCAGAGGCACCACTCCAGGCCCGGTGTTCACGGAGACACCTGGACCAC |
| | ACCCCCAGCGGAACCAGGGCGACGGTAGACACGGCACACCATGGTACCCATGGACACCTCCTAACCCCATGAC |
| | CGGTCCACCTGCCCTGGTGTTCAACAACTGCAGCGAGGTGCAGATCGGCAACTACAACAGCCTGGTGGCCCCT |
| | CCTAGGACCACCGCCAGCAGCAGCGCCAAGTACGATCAGGCACAGTTCGGCCGGGGCAGAGGTTGGCAGCCC |
| | TTCCACAAGGGAGGAATCAAGAAGGAGATCGAGGCCATTAAGAAGGAACAGGAAGCTATAAAGAAGAAGATT |
| | GAAGCTATCGAGAAGGAAATTGAGGCC |
| | (muRIPK3-IZ.Tri mer; TH1015 with no epitope tag)-nucleotide |
| 1416 | ATGGCCGCTCTGAAGTCATGGCTCTCAAGAAGTGTGACCAGCTTCTTCAGGTATAGGCAGTGCCTGTGCGTGCC |
| | GGTCGTTGCTAACTTTAAAAAACGCTGTTTCAGCGAGCTGATTCGCCCATGGCACAAAACCGTGACCATCGGGT |
| | TCGGAGTCACACTGTGCGCTGTCCCAATCGCACAAGCTGTGTATACGCTTACCTCACTTTACAGACAGTACACAT |
| | CTTTGCTGGGAAAGATGAATTCTGAGGAGGAAGACGAGGTGTGGCAAGTTATTATTGGCGCCAGAGCCGAAA |
| | TGACATCGAAGCATCAGGAATACCTGAAACTTGAGACCACATGGATGACGGCAGTCGGACTCTCCGAGATGGC |
| | AGCCGAAGCAGCCTACCAGACAGGTGCCGACCAGGCTAGCATCACAGCTCGGAACCATATCCAATTGGTAAAG |
| | CTGCAGGTCGAAGAGGTCCACCAACTAAGCCGAAAAGCCGAAACCAAACTGGCTGAAGCCCAGATTGAAGAA |
| | CTGCGGCAAAAAACCCAGGAAGAGGGCGAGGAGCGAGCCGAATCTGAGCAAGAAGCTTATCTGCGGGAAGA |
| | T |
| | (Diablo.3; TH2003 without epitope tag)-nucleotide |
| 1417 | ATGGGCTGCGTGTGCAGCAGCAACCCCGAGGACGACTGGATGGAGAACGGCGGCATCAAGAAGGAGATAGA |
| | AGCCATTAAGAAAGAGCAGGAGGCCATCAAGAAGAAGATCGAGGCCATCGAGAAGGAGATCGAAGCCGGCA |
| | GCGGCGGCGGCAGCGGCAGTGGCGGCGGCAGCGACCCCTTCCTGGTGCTGCTGCACAGCTTAAGCGGCAGCC |
| | TGAGCGGCAACGACCTGATGGAGCTGAAGTTCCTGTGTAGAGAGAGAGTGAGCAAGAGAAAGCTGGAGAGA |
| | GTGCAGAGCGGCCTGGACCTGTTCACCGTGCTGCTGGAGCAGAACGACCTGGAAAGAGGCCACACCGGCTTG |
| | CTGAGAGAGTTGCTGGCCTCACTGAGAAGACACGATCTGCTGCAGAGACTGGACGACTTCGAGGCCGGCACC |
| | GCCACCGCCGCCCCCCCCGGAGAAGCCGACCTGCAGGTGGCCTTCGACATCGTGTGCGACAACGTGGGCAGA |
| | GACTGGAAGAGATTGGCCAGAGAGCTGAAGGTGAGCGAGGCCAAGATGGACGGCATCGAGGAGAAGTACCC |
| | CAGAAGCCTGAGCGAGAGAGTGAGAGAGAGCCTGAAGGTGTGGAAGAACGCCGAGAAGAAGAACGCCAGC |
| | GTGGCTGGGCTGGTGAAGGCCCTGAGAACCTGCAGACTGAACCTGGTGGCCGATCTGGTGGAGGAGGCCCAG |
| | GAGAGCGTGAGCAAGAGCGAGAACATGAGCCCCGTGCTGAGAGACAGCACCGTGAGTAGCAGCGAGACCCC |
| | C |
| | (Myr(Lck)-IZ-L-msFADD; TH3002 without epitope tag)-nucleotide |
| 1418 | ATGGGCTGCGTGTGCAGCAGCAACCCCGAGGACGACTGGATGGAGAACGGCGGCATCAAAAAGGAGATCGA |
| | GGCCATCAAGAAGGAGCAGGAGGCCATCAAGAAGAAGATCGAGGCCATCGAGAAAGAGATAGAGGCCGGCA |
| | GCGGCGGCGGCAGCGGCAGCGGCGGCGGCAGCCCCGGCGAGGAGGACCTGTGCGCCGCCTTCAACGTGATC |
| | TGCGACAACGTGGGCAAGGACTGGAGAAGACTGGCCAGACAGCTGAAGGTGAGCGACACCAAGATCGACAG |
| | CATCGAGGACAGATACCCCAGAAACCTGACCGAGAGAGTGAGAGAGAGCCTGAGAATCTGGAAGAACACCGA |
| | GAAGGAGAACGCCACCGTGGCCCACCTGGTGGGCGCCCTGAGAAGCTGCCAGATGAACCTGGTGGCCGACCT |
| | GGTGCAGGAGGTGCAGCAGGCCAGAGACCTGCAGAACAGAAGCGGCGCCATGAGCCCCATGAGCTGGAACA |
| | GC |
| | (Myr(Lck)-IZ-L-huFADD-DD; TH3003 without epitope tag)-nucleotide |
| 1419 | ATGGGCTGCGTGTGCAGCAGCAACCCCGAGGACGACTGGATGGAGAACGGCGGCATCAAGAAAGAGATCGA |
| | GGCCATCAAAAAGGAGCAGGAGGCCATCAAGAAGAAGATCGAGGCCATCGAGAAGGAGATCGAGGCCGGCT |
| | CTGGCGGCGGCAGCGGCAGCGGCGGCGGCAGCCCCCCCGGCGAGGCCGACTTACAGGTGGCCTTCGACATCG |
| | TGTGCGACAACGTGGGCAGAGACTGGAAGAGACTGGCCAGAGAGCTGAAGGTGAGCGAGGCCAAGATGGAC |
| | GGCATCGAGGAGAAGTACCCCAGAAGCCTGAGCGAGAGAGTGAGAGAGAGCCTGAAGGTGTGGAAGAACGC |
| | CGAGAAGAAGAACGCCAGCGTGGCCGGCCTGGTGAAGGCCCTGAGAACCTGCAGACTGAACCTGGTGGCCGA |
| | CCTGGTGGAGGAGGCCCAGGAGAGCGTGAGCAAGAGCGAGAACATGAGCCCCGTGCTGAGAGACAGCACCG |
| | TGAGC |
| | (Myr(Lck)-IZ-L-msFADD-DD; TH3004 without epitope tag)-nucleotide |
| 1420 | ATGGGCCAGACCGTGACCACCCCCCTGAGCCTCACCCTGGATCACTGGGGCGGCATCAAGAAAGAGATCGAG |
| | GCCATCAAGAAGGAGCAGGAGGCCATCAAGAAGAAGATCGAAGCCATCGAGAAGGAGATCGAGGCCGGCAG |
| | CGGCGGCGGCAGCGGCAGCGGCGGCGGCAGCGACCCCTTCCTGGTGCTGCTGCACAGCGTGTCCAGCAGCCT |
| | GAGCAGCAGCGAGCTGACCGAGCTGAAGTTCCTGTGCCTGGGCAGAGTGGGCAAAAGAAAGCTGGAGAGAG |
| | TGCAGAGCGGCCTGGACCTCTTCAGCATGCTGCTGGAGCAGAACGACTTGGAGCCCGGCCACACCGAGCTGCT |
| | GAGAGAGCTGCTGGCCAGCCTGCGGAGACACGACCTGCTGAGAAGAGTGGATGACTTCGAGGCCGGCGCCGC |
| | CGCCGGCGCCGCCCCCGGCGAGGAGGACCTGTGCGCCGCCTTCAACGTGATCTGCGACAACGTGGGCAAGGA |
| | TTGGAGAAGATTAGCCAGACAGCTGAAGGTGAGTGACACCAAGATTGACAGCATCGAGGACAGATACCCCAG |
| | AAACCTGACCGAGAGAGTCAGAGAGAGCCTGAGAATCTGGAAGAATACCGAGAAGGAGAACGCCACCGTGG |
| | CCCACCTGGTGGGCGCCCTGAGAAGCTGCCAGATGAACCTGGTGGCCGACCTGGTGCAGGAGGTGCAGCAGG |
| | CCAGAGACCTGCAGAACAGAAGCGGCGCCATGAGCCCCATGAGCTGGAACAGCGACGCCAGCACCAGCGAGG |
| | CCAGC |
| | (Myr(MMSV)-IZ-L-huFADD; TH3005 without epitope tag)-nucleotide |
| 1421 | ATGGGCCAGACAGTGACCACCCCCCTGTCCCTGACCTTGGACCACTGGGGCGGCATCAAGAAGGAGATCGAG |
| | GCCATCAAGAAGGAGCAGGAGGCCATCAAAAAGAAGATCGAAGCCATTGAGAAGGAGATCGAGGCCGGAAG |
| | CGGGGGCGGCAGCGGCAGCGGCGGAGGAAGCGACCCCTTCCTGGTGCTGCTGCATAGCCTGTCAGGCAGCCT |
| | GAGCGGCAACGATCTGATGGAGCTGAAGTTCCTGTGCCGCGAGAGAGTGAGCAAGAGAAAGCTGGAGAGAG |
| | TACAGAGCGGCCTGGACCTGTTCACCGTGCTGCTGGAGCAGAATGACCTGGAGAGAGGCCACACCGGCTTGCT |
| | GAGAGAGTTGCTGGCCAGCCTGAGAAGGCACGACCTGCTGCAGAGACTGGACGACTTCGAGGCCGGCACCGC |
| | CACCGCCGCCCCCCCCGGCGAAGCGGACCTGCAGGTGGCCTTCGACATCGTGTGCGACAACGTGGGCAGAGA |
| | CTGGAAGAGACTGGCCAGAGAACTGAAGGTGAGCGAGGCCAAAATGGACGGCATCGAGGAGAAGTACCCCA |
| | GAAGCCTGAGCGAGAGAGTGAGAGAGAGCCTGAAGGTGTGGAAGAACGCCGAGAAGAAGAACGCCAGCGT |
| | GGCCGGCCTGGTGAAGGCCCTGAGAACATGCAGACTGAACCTGGTGGCCGATCTTGTGGAGGAGGCCCAGGA |
| | GAGCGTGAGCAAGAGCGAAAACATGAGCCCCGTGCTGAGAGACAGCACCGTGAGCAGCAGCGAGACCCCC |
| | (Myr(MMSV)-IZ-L-msFADD; TH3006 without epitope tag)-nucleotide |
| 1422 | ATGGGCCAGACCGTGACCACCCCCCTGAGCCTGACCCTGGACCACTGGGGCGGCATCAAGAAGGAGATCGAG |
| | GCCATCAAGAAGGAGCAGGAGGCCATCAAGAAGAAGATTGAGGCTATCGAGAAGGAGATCGAGGCCGGCAG |
| | CGGCGGCGGCAGCGGCAGCGGCGGCGGCAGCCCCGGCGAGGAGGACCTGTGCGCCGCCTTCAACGTGATCT |
| | GCGACAACGTGGGCAAGGACTGGAGAAGACTGGCCAGACAGCTGAAGGTGAGCGACACCAAGATCGACAGC |
| | ATCGAGGACAGATACCCCAGAAACCTGACCGAGAGAGTGAGAGAGAGCCTGAGAATCTGGAAGAACACCGAG |
| | AAGGAGAACGCCACCGTGGCCCACCTGGTGGGCGCCCTGAGAAGCTGCCAGATGAACCTGGTGGCCGACCTG |
| | GTGCAGGAGGTGCAGCAGGCCAGAGACCTGCAGAACAGAAGCGGCGCCATGAGCCCCATGAGCTGGAACAG |
| | C |
| | (Myr(MMSV)-IZ-L-huFADD-DD; TH3007 without epitope tag)-nucleotide |
| 1423 | UCAAGCUUUUGGACCCUCGUACAGAAGCUAAUACGACUCACUAUAGGGAAAUAAGAGAGAAAAGAAGAG |
| | UAAGAAGAAAUAUAAGAGCCACC |
| | (5′ UTR) |
| 1424 | UGAUAAUAGGCUGGAGCCUCGGUGGCCAUGCUUCUUGCCCCUUGGGCCUCCCCCCAGCCCCUCCUCCCCU |
| | UCCUGCACCCGUACCCCCGUGGUCUUUGAAUAAAGUCUGAGUGGGCGGC |
| | (3′ UTR) |
| 1425 | UGAUAAUAGGCUGGAGCCUCGGUGGCCAUGCUUCUUGCCCCUUGGGCCCAAACACCAUUGUCACACUCCA |
| | UCCCCCCAGCCCCUCCUCCCCUUCCUCCAUAAAGUAGGAAACACUACAUGCACCCGUACCCCCGUGGUCUU |
| | UGAAUAAAGUCUGAGUGGGCGGC |
| | (3′ UTR with mi-122 and mi-142-3p sites) |
| 1426 | GGAAGCGGAGCUACUAACUUCAGCCUGCUGAAGCAGGCUGGAGACGUGGAGGAGAACCCUGGACCU |
| | (Nucleotide sequence encoding 2A peptide) |
| 1427 | UCCGGACUCAGAUCCGGGGAUCUCAAAAUUGUCGCUCCUGUCAAACAAACUCUUAACUUUGAUUUACUC |
| | AAACUGGCUGGGGAUGUAGAAAGCAAUCCAGGUCCACUC |
| | (Nucleotide sequence encoding 2A peptide) |
| 1428 | AUGGAACGCCCCCCUGGACUGAGGCCUGGAGCAGGAGGACCCUGGGAAAUGCGCGAACGGCUGGGUACU |
| | GGUGGUUUCGGCAACGUGUGCCUCUACCAGCAUCGGGAGUUGGACCUGAAGAUCGCCAUCAAGUCCUGC |
| | CGCCUGGAGCUGUCGACCAAGAACCGGGAACGCUGGUGUCAUGAAAUCCAGAUUAUGAAAAAGCUGAAC |
| | CACGCUAACGUGGUCAAAGCUUGCGACGUGCCCGAAGAACUGAAUAUCCUGAUCCACGAUGUGCCCCUCC |
| | UCGCAAUGGAGUACUGCAGCGGAGGCGAUCUCCGGAAGCUGCUCAACAAGCCGGAGAACUGCUGUGGCC |
| | UUAAAGAGAGCCAGAUUCUGAGCCUUCUGUCGGACAUCGGCUCGGGUAUCCGAUAUCUUCACGAGAACA |
| | AGAUUAUUCACAGAGAUCUGAAGCCAGAGAACAUCGUGCUGCAAGAUGUCGGAGGAAAGAUCAUUCAUA |
| | AGAUCAUCGACCUGGGAUACGCCAAGGACGUGGAUCAAGGCGAACUGUGCACCGAAUUCGUGGGAACCC |
| | UCCAGUACCUGGCCCCGGAACUGUUCGAAAACAAACCCUACACCGCCACCGUGGACUACUGGUCCUUUGG |
| | AACUAUGGUGUUCGAGUGUAUAGCUGGCUACCGGCCAUUUCUCCAUCACUUGCAGCCCUUCACCUGGCA |
| | CGAAAAGAUCAAGAAGAAGGACCCCAAGUGCAUUUUCGCGUGCGAAGAGAUGUCGGGGGAAGUGCGCUU |
| | CUCGUCCCACUUGCCCCAGCCCAACUCCCUGUGCUCCCUGGUGGUCGAACCGAUGGAAAACUGGCUGCAA |
| | CUGAUGCUGAACUGGGAUCCUCAACAGCGCGGUGGACCAGUGGAUCUGACUCUGAAGCAGCCCAGAUGC |
| | UUCGUGCUGAUGGACCAUAUCCUGAACCUCAAGAUCGUCCACAUCCUGAACAUGACCUCCGCCAAGAUCA |
| | UUUCCUUCCUCCUCCCGCCCGAUGAGAGCCUGCACUCACUGCAGUCCAGAAUCGAGAGGGAAACCGGUAU |
| | UAACACUGGGUCACAGGAACUCCUGUCCGAAACCGGAAUCUCUCUGGACCCUCGCAAGCCAGCAUCCCAG |
| | UGCGUCCUGGAUGGGGUCAGGGGAUGCGACUCGUACAUGGUCUACCUCUUCGAUAAGUCAAAGACCGUC |
| | UACGAGGGACCCUUUGCCAGCCGGAGCCUGUCAGACUGCGUGAACUACAUCGUGCAGGACUCUAAGAUU |
| | CAGCUGCCAAUUAUCCAGCUCCGGAAAGUCUGGGCAGAAGCGGUGCACUACGUGUCCGGACUGAAAGAG |
| | GACUACUCCCGGCUGUUCCAGGGCCAGAGGGCAGCCAUGCUGUCCCUGCUCCGCUACAACGCCAACCUCAC |
| | GAAGAUGAAGAACACCCUGAUCUCCGCGUCACAACAACUGAAGGCCAAGCUGGAAUUCUUCCACAAGUCC |
| | AUUCAAUUGGAUCUGGAGCGGUACUCCGAGCAGAUGACUUACGGCAUUAGCUCCGAAAAGAUGCUCAAG |
| | GCCUGGAAGGAGAUGGAGGAGAAGGCCAUUCAUUAUGCCGAAGUGGGGGUGAUCGGAUACCUGGAGGA |
| | UCAGAUCAUGUCCCUUCAUGCCGAGAUUAUGGAACUCCAGAAGUCCCCGUACCGGAGGCAGGGCGAUUU |
| | GAUGGAGAGCUUGGAACAACGCGCCAUCGACCUGUACAAGCAGCUCAAGCACAGACCGAGCGACCACUCG |
| | UACUCCGACUCGACUGAGAUGGUGAAAAUUAUCGUGCACACCGUGCAGUCCCAAGACCGGGUCCUGAAG |
| | GAGCUGUUCGGACACCUGAGCAAGCUGCUGGGGUGCAAGCAAAAGAUCAUUGACCUUCUGCCAAAAGUG |
| | GAGGUGGCCCUGAGCAACAUUAAGGAAGCCGACAACACCGUGAUGUUCAUGCAGGGCAAGCGGCAGAAG |
| | GAGAUCUGGCAUCUUCUCAAGAUCGCGUGUACCCAGGCUGCAGCGAGAGCCUUGGUGGGCGCUGCCCUG |
| | GAAGGUGCCGUGGCACCACAGGCCGCUGCUUGGCUGCCUCCUGCUGCUGCUGAGCACGAUCACGCACUGG |
| | CCUGCGUGGUGGCACCGCAGGACGGAGAGGCUGCCGCGCAGAUGAUCGAGGAAAACCUGAACUGCCUGG |
| | GUCACCUGGCUGCCAUCAUCCACGAAGCCAACGAGGAGCAAGGAAACAGCAUGAUGAAUCUCGACUGGAG |
| | CUGGCUGACUGAG |
| | Human constitutively active IKK alpha (PEST mutation) P.4013/4014 without epitope tag- |
| | nucleotide |
| 1429 | AUGGAAAGACCGCCUGGAUUGCGACCUGGAGCCGGAGGACCCUGGGAAAUGAGAGAGAGAUUGGGUACU |
| | GGAGGCUUCGGAAAUGUCUCGCUGUACCAGCACCGCGAGCUCGACCUGAAGAUCGCGAUCAAGUCCUGU |
| | CGCCUGGAGCUGUCCAGCAAGAACAGAGAGCGGUGGUGCCACGAGAUCCAGAUUAUGAAGAAGCUGGAC |
| | CAUGCCAACGUCGUGAAGGCUUGCGAUGUCCCGGAGGAACUCAAUUUCCUUAUUAACGACGUGCCGCUU |
| | CUCGCGAUGGAGUACUGCUCAGGCGGCGACUUGCGCAAGCUGCUUAACAAGCCCGAAAACUGCUGCGGU |
| | CUGAAGGAAUCCCAAAUUCUGUCACUCCUGUCCGAUAUUGGCUCAGGAAUCCGCUACCUUCAUGAGAAU |
| | AAGAUCAUCCACCGCGACCUGAAGCCUGAGAACAUUGUGCUGCAGGAUGUCGGGGGAAAGACUAUCCACA |
| | AGAUAAUCGACCUGGGAUACGCCAAGGACGUCGAUCAAGGGGAACUGUGCACCGAAUUCGUGGGGACUC |
| | UCCAGUACUUGGCCCCCGAACUGUUUGAAAACAAGCCCUACACCGCCACCGUGGAUUACUGGUCCUUCGG |
| | GACUAUGGUGUUCGAGUGUAUUGCCGGCUAUCGCCCCUUUCUGCACCACCUCCAGCCCUUUACUUGGCA |
| | CGAAAAGAUCAAGAAGAAGGAUCCGAAGUGCAUCUUCGCUUGCGAAGAGAUGACCGGAGAAGUCCGGUU |
| | UUCCAGCCAUCUGCCUCAGCCGAACUCCCUGUGUUCCCUGAUUGUGGAACCCAUGGAGAGCUGGUUGCA |
| | GCUCAUGCUCAACUGGGAUCCGCAGCAACGCGGUGGCCCAAUCGAUCUUACCCUUAAGCAGCCUCGGUGC |
| | UUCGCGCUGAUGGACCACAUCCUCAAUCUGAAGAUCGUGCACAUCCUGAACAUGACUUCCGCCAAGAUCA |
| | UCUCCUUCCUGCUGCCGUGCGACGAAAGCCUGCACUCACUGCAGAGCCGGAUCGAACGGGAGACAGGCAU |
| | AAACACGGGAUCGCAAGAACUGCUGUCCGAAACCGGCAUCUCCCUGGACCCACGGAAGCCUGCCUCCCAAU |
| | GCGUCCUGGACGGAGUGCGGGGUUGCGACUCAUACAUGGUGUACCUCUUCGAUAAGUCAAAGACCGUGU |
| | AUGAAGGACCCUUCGCCUCCCGCUCCCUGAGCGACUGCGUGAACUACAUCGUGCAGGACUCGAAGAUCCA |
| | GCUGCCGAUUAUCCAGCUUCGGAAGGUCUGGGCGGAGGCUGUGCACUACGUGUCCGGUUUGAAAGAGG |
| | AUUAUAGCCGCCUGUUCCAGGGACAGAGAGCCGCCAUGCUGUCCCUCCUCCGGUACAACGCCAACCUGAC |
| | CAAGAUGAAGAACACCCUGAUCAGCGCCUCGCAGCAGCUGAAGGCCAAGCUGGAGUUCUUCCGGAAGUCG |
| | AUCCAGCUCGACCUCGAAAGGUACUCAGAACAGAUGACCUACGGAAUUUCCUCCGAGAAGAUGCUGAAAG |
| | CCUGGAAGGAAAUGGAGGAGAAGGCCAUUCACUACUCCGAAGUGGGCGUCAUUGGCUACUUGGAGGACC |
| | AAAUCAUGUCUCUGCACACCGAAAUCAUGGAACUCCAGAAGUCGCCUUACGGACGACGCCAAGGGGACCU |
| | GAUGGAGAGCCUGGAACAGCGGGCCAUCGAUCUGUACAAGCAACUGAAGCAUAGGCCGCCCGACCAUCUC |
| | UACUCCGACUCGACUGAAAUGGUGAAGAUUAUUGUGCAUACAGUGCAGAGCCAGGACAGAGUGCUGAAG |
| | GAGCUGUUCGGCCACCUGUCCAAGCUCCUGGGUUGCAAGCAGAAGAUUAUCGAUCUGUUGCCCAAGGUG |
| | GAAGUGGCCCUGUCUAACAUCAAAGAAGCCGACAACACUGUGAUGUUUAUGCAAGGAAAGCGGCAGAAA |
| | GAAAUCUGGCACCUUCUGAAAAUCGCGUGCACCCAGGCUGCAGCUAGGGCACUCGUGGGUGCAGCGCUU |
| | GAAGGCGCCGUGGCACCUCCUGUCGCUGCCUGGUUGCCACCCGCGCUUGCUGACAGAGAGCACCCACUGA |
| | CUUGUGUGGUGGCCCCACAGGACGGAGAAGCACUGGCCCAGAUGAUUGAGGAGAACCUGAACUGUCUGG |
| | GACACCUUGCCGCCAUUAUCCGGGAGGCCAACGAGGACCAGUCCUCGUCCCUGAUGUCCCUGGAUUGGUC |
| | AUGGCUCGCUGAA |
| | Mouse constitutively active IKK alpha (PEST mutation) P.4017/4018 without epitope tag- |
| | nucleotide |
| 1430 | AUGAGCUGGAGCCCUUCACUGCCAACCCAAACCUGUGGAGCCUGGGAAAUGAAAGAAAGACUGGGAACCG |
| | GAGGUUUCGGCAACGUGAUCCGCUGGCAUAACCAGGCCACUGGGGAGCAGAUUGCCAUCAAGCAGUGCC |
| | GGCAGGAGCUGUCCCCGAAGAACCGCAACCGGUGGUGCCUGGAAAUCCAGAUCAUGCGGCGGCUUAACCA |
| | CCCCAACGUGGUCGCCGCGAGAGAUGUGCCGGAGGGCAUGCAAAACCUGGCCCCCAACGAUCUCCCGCUG |
| | UUGGCGAUGGAGUAUUGCCAGGGUGGCGAUCUGCGGCGCUACCUGAAUCAAUUCGAGAACUGCUGCGG |
| | UCUGCGCGAAGGAGCUGUGCUUACGCUGCUCUCGGACAUCGCCUCGGCGCUGAGAUACCUCCACGAAAAU |
| | CGGAUCAUCCACCGAGAUCUCAAGCCGGAAAACAUUGUGCUUCAGCAAGGGGAAAAGCGCCUCAUCCAUA |
| | AGAUCAUCGAUCUCGGCUACGCCAAGGAGUUGGACCAGGGGGAGCUCUGCACUGAAUUCGUGGGAACUC |
| | UGCAGUACUUGGCGCCCGAACUGCUGGAGCAACAGAAGUACACUGUGACCGUGGACUACUGGUCCUUUG |
| | GAACCCUGGCCUUCGAGUGCAUUACUGGCUUCCGGCCUUUCCUUCCAAACUGGCAGCCGGUGCAGUGGC |
| | ACUCAAAGGUCCGCCAGAAGUCCGAAGUGGACAUCGUGGUGUCCGAGGACUUGAACGGCGCCGUGAAGU |
| | UCUCGUCCUCCCUGCCCUUCCCGAACAACCUCAACUCCGUGCUGGCCGAGAGGCUGGAAAAGUGGCUGCA |
| | GCUUAUGCUGAUGUGGCACCCUAGACAGCGCGGAACUGAUCCGCAGUACGGCCCGAACGGCUGUUUUAG |
| | GGCCCUGGACGACAUUCUGAACCUGAAACUCGUCCACGUGCUUAACAUGGUCACCGGUACCGUCCAUACC |
| | UAUCCGGUCACCGAGGACGAAUCCCUGCAGUCCCUCAAGACUCGGAUUCAGGAGAAUACCGGCAUUCUGG |
| | AAACCGACCAGGAGCUGCUGCAGAAGGCCGGACUGGUGCUGCUCCCCGAUAAGCCCGCAACCCAGUGCAU |
| | CUCAGACUCCAAGACCAACGAGGGCCUGACUCUCGACAUGGACCUGGUGUUCCUGCUCGACAACAGCAAG |
| | AUCAACUACGAAACCCAAAUUACCCCUAGACCACCACCUGAAUCCGUGAGCUGCAUACUGCAGGAGCCCAA |
| | GCGCAACCUCUCCUUCUUCCAACUCCGGAAGGUCUGGGGCCAAGUGUGGCACUCCAUUCAGACUCUGAAG |
| | GAAGAUUGUAACAGGCUGCAGCAGGGACAGAGAGCCGCCAUGAUGAGCCUUCUGAGGAACAACUCUUGC |
| | CUGUCAAAGAUGAAGAACGCCAUGGCUUCCACCGCGCAGCAGUUGAAGGCGAAGCUGGACUUCUUUAAG |
| | ACCUCCAUCCAAAUCGACCUGGAGAAGUACAAGGAACAGACUGAGUUCGGGAUUACGAGCGAUAAACUCC |
| | UGCUCGCUUGGCGGGAAAUGGAGCAAGCAGUGGAGCAGUGCGGACGGGAGAACGACGUCAAGCAUCUCG |
| | UGGAGCGGAUGAUGGCGCUGCAGACCGACAUUGUCGACUUGCAGCGCUCUCCAAUGGGACGGAAGCAGG |
| | GAGGGACUCUGGACGAUCUGGAGGAACAGGCCCGGGAACUGUACAGAAAGCUGAGGGAGAAGCCCCGGG |
| | AUCAAAGAACCGAAGGAGACUCGCAAGAGAUGGUGCGCCUGCUGCUGCAGGCGAUCCAGUCCUUCGAGA |
| | AGAAGGUCCGCGUGAUCUACACUCAGCUGUCCAAGACCGUGGUCUGUAAACAGAAGGCCCUGGAACUGC |
| | UCCCGAAAGUGGAAGAAGUGGUGUCGCUCAUGAAUGAGGACGAGAGAACCGUGGUGCGCCUCCAAGAAA |
| | AGCGGCAGAAGGAACUCUGGAACCUCCUCAAGAUUGCCUGCUCGAAAGUGCGGGGACCUGUGGCUGGUG |
| | CUCCUGACGCCAUGAACGUGGCCAGGCUUGCUCACCCUGGCCAACUUAUGGCCCAGCCUGCAUCCGCCUG |
| | UGACGCACUGCCCGAGUCGGACAAGAAGGCCGAAGAACUGGUCGCCGAAGCCCACGCACUGUGCAGCCGC |
| | CUGGAAAGCGCGCUGCAGGACACCGUGAAGGAGCAGGACCGCAGCUUUACCACUCUUGAUUGGUCCUGG |
| | CUGCAAAUGGAGGACGAAGAACGGUGCUCCCUGGAACAGGCCUGCGAC |
| | Mouse constitutively active IKK beta (PEST mutation) P.4019/4020 without epitope tag- |
| | nucleotide |
| 1431 | AUGACCGAGUACAAGCUGGUGGUGGUGGGCGCCGACGGCGUGGGCAAGAGCGCCCUGACCAUCCAGCUG |
| | AUCCAG |
| | (KRAS G12D 25 mer nucleotide sequence) |
| 1432 | AUGACCGAGUACAAGCUGGUGGUGGUGGGCGCCGUGGGCGUGGGCAAGAGCGCCCUGACCAUCCAGCUG |
| | AUCCAG |
| | (KRAS G12V 25 mer nucleotide sequence) |
| 1433 | AUGACCGAGUACAAGCUGGUGGUGGUGGGCGCCGGCGACGUGGGCAAGAGCGCCCUGACCAUCCAGCUG |
| | AUCCAG |
| | (KRAS G13D 25 mer nucleotide sequence) |
| 1434 | AUGACCGAGUACAAGUUAGUGGUUGUGGGCGCCGACGGCGUGGGCAAGAGCGCCCUCACCAUCCAGCUU |
| | AUCCAGAUGACGGAAUAUAAGUUAGUAGUAGUGGGAGCCGACGGUGUCGGCAAGUCCGCUUUGACCAU |
| | UCAACUUAUUCAGAUGACAGAGUAUAAGCUGGUCGUUGUAGGCGCAGACGGCGUUGGAAAGUCGGCAC |
| | UGACGAUCCAGUUGAUCCAG |
| | (KRAS G12D 25 mer^{∧}3 nucleotide sequence) |
| 1435 | AUGACCGAGUACAAGCUCGUCGUGGUGGGCGCCGUGGGCGUGGGCAAGAGCGCCCUAACCAUCCAGUUG |
| | AUCCAGAUGACCGAAUAUAAGCUCGUGGUAGUCGGAGCGGUGGGCGUUGGCAAGUCAGCGCUAACAAUA |
| | CAACUAAUCCAAAUGACCGAAUACAAGCUAGUUGUAGUCGGUGCCGUCGGCGUUGGAAAGUCAGCCCUU |
| | ACAAUUCAGCUCAUUCAG |
| | (KRAS G12V 25 mer^{∧}3 nucleotide sequence) |
| 1436 | AUGACCGAGUACAAGCUCGUAGUGGUUGGCGCCGGCGACGUGGGCAAGAGCGCCCUAACCAUCCAGCUCA |
| | UCCAGAUGACAGAAUAUAAGCUUGUGGUUGUGGGAGCAGGAGACGUGGGAAAGAGUGCGUUGACGAUU |
| | CAACUCAUACAGAUGACCGAAUACAAGUUGGUGGUGGUCGGCGCAGGUGACGUUGGUAAGUCUGCACUA |
| | ACUAUACAACUGAUCCAG |
| | (KRAS G13D 25 mer^{∧}3 nucleotide sequence) |
| 1437 | AUGACCGAGUACAAGCUGGUGGUGGUGGGCGCCUGCGGCGUGGGCAAGAGCGCCCUGACCAUCCAGCUG |
| | AUCCAG |
| | (KRAS G12C 25 mer nucleotide sequence) |
| 1438 | AUGACCGAGUACAAGCUCGUGGUUGUUGGCGCCUGCGGCGUGGGCAAGAGCGCCCUCACCAUCCAGCUC |
| | AUCCAGAUGACAGAGUAUAAGUUAGUCGUUGUCGGAGCUUGCGGAGUUGGAAAGUCGGCGCUCACCAU |
| | UCAACUCAUACAAAUGACAGAAUAUAAGUUAGUGGUGGUGGGUGCGUGUGGCGUUGGCAAGAGUGCGC |
| | UUACUAUCCAGCUCAUUCAG |
| | (KRAS G12C 25 mer^{∧}3 nucleotide sequence) |
| 1439 | AUGACCGAGUACAAGCUGGUGGUGGUGGGCGCCGGCGGCGUGGGCAAGAGCGCCCUGACCAUCCAGCUG |
| | AUCCAG |
| | (KRAS WT 25 mer nucleotide sequence) |
| 1440 | GGGAAAUAAGAGAGAAAAGAAGAGUAAGAAGAAAUAUAAGAGCCACC |
| | (5′ UTR sequence; no promoter) |
| 1441 | AUGACCGAGUACAAGCUCGUUGUAGUCGGCGCCGACGGCGUGGGCAAGAGCGCCUUGACCAUCCAGUUG |
| | AUCCAGAUGACCGAAUAUAAGUUGGUGGUGGUAGGCGCAGUGGGAGUUGGCAAGUCAGCACUCACAAU |
| | UCAGCUCAUUCAAAUGACAGAAUACAAGUUAGUCGUUGUAGGAGCAGGCGACGUCGGCAAGAGUGCCUU |
| | AACCAUUCAACUAAUCCAG |
| | (KRAS(G12D G12V G13D) 75 mer “3MUT” nt. seq) |
| 1442 | AUGCCUCACAGCAGCCUGCACCCUAGCAUCCCUUGCCCUAGAGGCCACGGCGCCCAGAAGGCCGCCCUGGU |
| | GCUGCUGAGCGCCUGCCUGGUGACCCUGUGGGGCCUGGGCGAGCCUCCUGAGCACACCCUGAGAUACCU |
| | GGUGCUGCACCUGGCCAGCCUGCAGCUGGGCCUGCUGCUGAACGGCGUGUGCAGCCUGGCCGAGGAGCU |
| | GAGACACAUCCACAGCAGAUACAGAGGCAGCUACUGGAGAACCGUGAGAGCCUGCCUGGGCUGCCCUCUG |
| | AGAAGAGGCGCCCUGCUGCUGCUGAGCAUCUACUUCUACUACAGCCUGCCUAACGCCGUGGGCCCUCCUU |
| | UCACCUGGAUGCUGGCCCUGCUGGGCCUGAGCCAGGCCCUGAACAUCCUGCUGGGCCUGAAGGGCCUGG |
| | CCCCUGCCGAGAUCAGCGCCGUGUGCGAGAAGGGCAACUUCAACGUGGCCCACGGCCUGGCCUGGAGCUA |
| | CUACAUCGGCUACCUGAGACUGAUCCUGCCUGAGCUGCAGGCCAGAAUCAGAACCUACAACCAGCACUAC |
| | AACAACCUGCUGAGAGGCGCCGUGAGCCAGAGACUGUACAUCCUGCUGCCUCUGGACUGCGGCGUGCCU |
| | GACAACCUGAGCAUGGCCGACCCUAACAUCAGAUUCCUGGACAAGCUGCCUCAGCAGACCGGCGACCACGC |
| | CGGCAUCAAGGACAGAGUGUACAGCAACAGCAUCUACGAGCUGCUGGAGAACGGCCAGAGAGCCGGCACC |
| | UGCGUGCUGGAGUACGCCACCCCUCUGCAGACCCUGUUCGCCAUGAGCCAGUACAGCCAGGCCGGCUUCA |
| | GCAGAGAGGACACCCUGGAGCAGGCCAAGCUGUUCUGCAGAACCCUGGAGGACAUCCUGGCCGACGCCCC |
| | UGAGAGCCAGAACAACUGCAGACUGAUCGCCUACCAGGAGCCUGCCGACGACAGCAGCUUCAGCCUGAGC |
| | CAGGAGGUGCUGAGACACCUGAGACAGGAGGAGAAGGAGGAGGUGACCGUGGGCAGCCUGAAGACCAGC |
| | GCCGUGCCUAGCACCAGCACCAUGAGCCAGGAGCCUGAGCUGCUGAUCAGCGGCAUGGAGAAGCCUCUGC |
| | CUCUGAGAACCGACUUCAGC |
| | (Hu STING(R284U); no epitope tag; nucleotide sequence) |
| 1443 | AUGCCCCACAGCAGCCUGCACCCCUCCAUCCCCUGUCCCAGAGGCCACGGCGCCCAGAAGGCCGCCCUGGU |
| | GCUGCUGAGCGCCUGCCUGGUGACCUUAUGGGGCCUGGGCGAGCCCCCCGAGCACACCCUGAGAUACCUG |
| | GUCCUGCACCUGGCCAGCCUCCAGCUGGGCCUGCUGCUCAACGGCGUGUGUAGCCUGGCCGAGGAGCUG |
| | AGACACAUCCACAGCAGAUACAGAGGCAGCUACUGGAGAACCGUGAGAGCCUGCCUGGGUUGCCCACUGA |
| | GAAGAGGAGCUCUGCUGCUGCUGAGCAUCUACUUCUACUACUCGCUGCCCAACGCUGUGGGCCCCCCCUU |
| | CACCUGGAUGCUGGCCCUGCUGGGUCUGAGCCAGGCCCUGAACAUCCUCCUGGGCCUGAAGGGCCUGGCC |
| | CCCGCCGAGAUAAGCGCCGUUUGCGAGAAGGGCAACUUCAACGUGGCCCAUGGCCUGGCCUGGAGCUACU |
| | ACAUCGGCUACUUACGCCUGAUCCUGCCCGAGCUGCAGGCCAGAAUCAGAACCUACAACCAGCAUUACAAC |
| | AACCUGCUGAGAGGCGCCGUGAGCCAGAGACUGUAUAUCCUGCUGCCCCUGGACUGCGGCGUGCCCGACA |
| | ACCUGAGCAUGGCCGACCCCAACAUCAGAUUCCUGGACAAGCUCCCCCAGCAGACCGGCGACCACGCCGGA |
| | AUCAAAGACAGAGUGUAUAGCAACAGCAUCUACGAGCUGCUGGAGAACGGCCAGAGAGCCGGCACCUGCG |
| | UACUGGAGUACGCCACCCCCUUGCAGACCCUGUUUGCCAUGAGCCAGUACAGCCAGGCCGGCUUCAGCAG |
| | AGAGGACAUGCUGGAGCAGGCCAAGCUGUUCUGCAGAACCCUGGAGGACAUCCUGGCCGACGCCCCCGAG |
| | AGCCAGAACAACUGCAGACUGAUCGCCUACCAAGAGCCCGCCGACGACAGCAGCUUCAGCUUAAGCCAGGA |
| | GGUGCUGAGACAUCUGAGACAGGAGGAGAAGGAGGAGGUGACCGUGGGCAGCCUCAAGACCAGCGCUGU |
| | GCCCUCUACCAGCACCAUGAGCCAGGAGCCCGAGCUGCUGAUCAGCGGCAUGGAGAAGCCCCUGCCCCUG |
| | AGAACAGACUUCAGC |
| | (hu STING (R284M); no epitope tag; nucleotide sequence) |
| 1444 | AUGCCCCAUAGCAGCCUGCACCCCAGCAUCCCCUGCCCCAGAGGCCACGGCGCCCAGAAGGCCGCCCUGGU |
| | CCUGCUGAGCGCAUGCCUGGUCACCCUGUGGGGCCUGGGCGAGCCCCCCGAGCACACCCUGAGAUACCUG |
| | GUGCUGCACCUCGCCAGCCUGCAGCUGGGCCUGCUGCUGAACGGCGUGUGCAGCCUGGCCGAGGAGCUG |
| | AGACACAUCCACAGCAGAUAUAGAGGCAGCUACUGGAGAACCGUGAGAGCUUGCCUCGGCUGCCCCCUGA |
| | GAAGAGGCGCCCUGCUGCUGCUGAGCAUCUACUUUUACUACAGCCUGCCCAACGCUGUGGGCCCCCCUUU |
| | CACGUGGAUGCUCGCCCUGCUGGGACUGAGCCAGGCCCUGAACAUCCUGCUGGGCCUUAAGGGCCUAGCC |
| | CCCGCCGAGAUCAGCGCCGUGUGCGAGAAGGGCAACUUCAAUGUGGCCCACGGCCUGGCCUGGAGCUACU |
| | ACAUCGGCUACCUGAGACUGAUCCUGCCCGAGCUGCAGGCCAGAAUCAGAACCUACAAUCAGCACUACAAC |
| | AACCUGCUGAGAGGCGCCGUGAGCCAGAGACUGUACAUCCUGCUGCCCCUGGACUGCGGCGUGCCCGACA |
| | ACCUCAGCAUGGCCGACCCCAACAUCAGAUUCCUGGACAAGCUGCCCCAGCAGACCGGCGACCACGCCGGC |
| | AUCAAGGAUCGCGUGUACAGCAACAGCAUCUACGAGCUGCUGGAAAACGGCCAGAGAGCCGGAACCUGCG |
| | UGCUGGAGUACGCCACACCCCUGCAGACCCUGUUCGCCAUGAGCCAGUACAGCCAGGCCGGCUUCAGCAG |
| | AGAGGACAAGCUGGAGCAGGCCAAGCUGUUCUGCAGAACCCUGGAGGAUAUCCUCGCCGACGCCCCCGAG |
| | AGCCAGAACAACUGCAGGCUGAUCGCGUACCAGGAGCCCGCUGACGACAGCAGCUUUAGCCUGAGCCAGG |
| | AGGUGCUGAGACAUCUGCGUCAAGAGGAAAAGGAGGAGGUGACCGUGGGCUCCCUGAAGACCAGCGCCG |
| | UGCCCAGCACCAGCACCAUGAGCCAGGAGCCCGAGCUGCUGAUCAGCGGCAUGGAGAAGCCACUGCCCCUC |
| | AGAACCGACUUCAGC |
| | (Hu STING (R284K); no epitope tag; nucleotide sequence) |
| 1445 | AUGCCUCACAGCAGCCUGCACCCUAGCAUCCCUUGCCCUAGAGGCCACGGCGCCCAGAAGGCCGCCCUGGU |
| | GCUGCUGAGCGCCUGCCUGGUGACCCUGUGGGGCCUGGGCGAGCCUCCUGAGCACACCCUGAGAUACCU |
| | GGUGCUGCACCUGGCCAGCCUGCAGCUGGGCCUGCUGCUGAACGGCGUGUGCAGCCUGGCCGAGGAGCU |
| | GAGACACAUCCACAGCAGAUACAGAGGCAGCUACUGGAGAACCGUGAGAGCCUGCCUGGGCUGCCCUCUG |
| | AGAAGAGGCGCCCUGCUGCUGCUGAGCAUCUACUUCUACUACAGCCUGCCUAACGCCGUGGGCCCUCCUU |
| | UCACCUGGAUGCUGGCCCUGCUGGGCCUGAGCCAGGCCCUGAACAUCCUGCUGGGCCUGAAGGGCCUGG |
| | CCCCUGCCGAGAUCAGCGCCGUGUGCGAGAAGGGCAACUUCAGCGUGGCCCACGGCCUGGCCUGGAGCUA |
| | CUACAUCGGCUACCUGAGACUGAUCCUGCCUGAGCUGCAGGCCAGAAUCAGAACCUACAACCAGCACUAC |
| | AACAACCUGCUGAGAGGCGCCGUGAGCCAGAGACUGUACAUCCUGCUGCCUCUGGACUGCGGCGUGCCU |
| | GACAACCUGAGCAUGGCCGACCCUAACAUCAGAUUCCUGGACAAGCUGCCUCAGCAGACCGGCGACCACGC |
| | CGGCAUCAAGGACAGAGUGUACAGCAACAGCAUCUACGAGCUGCUGGAGAACGGCCAGAGAGCCGGCACC |
| | UGCGUGCUGGAGUACGCCACCCCUCUGCAGACCCUGUUCGCCAUGAGCCAGUACAGCCAGGCCGGCUUCA |
| | GCAGAGAGGACAGACUGGAGCAGGCCAAGCUGUUCUGCAGAACCCUGGAGGACAUCCUGGCCGACGCCCC |
| | UGAGAGCCAGAACAACUGCAGACUGAUCGCCUACCAGGAGCCUGCCGACGACAGCAGCUUCAGCCUGAGC |
| | CAGGAGGUGCUGAGACACCUGAGACAGGAGGAGAAGGAGGAGGUGACCGUGGGCAGCCUGAAGACCAGC |
| | GCCGUGCCUAGCACCAGCACCAUGAGCCAGGAGCCUGAGCUGCUGAUCAGCGGCAUGGAGAAGCCUCUGC |
| | CUCUGAGAACCGACUUCAGC |
| | (Hu STING(N154S); no epitope tag; nucleotide sequence) |
| 1446 | AUGCCUCACAGCAGCCUGCACCCUAGCAUCCCUUGCCCUAGAGGCCACGGCGCCCAGAAGGCCGCCCUGGU |
| | GCUGCUGAGCGCCUGCCUGGUGACCCUGUGGGGCCUGGGCGAGCCUCCUGAGCACACCCUGAGAUACCU |
| | GGUGCUGCACCUGGCCAGCCUGCAGCUGGGCCUGCUGCUGAACGGCGUGUGCAGCCUGGCCGAGGAGCU |
| | GAGACACAUCCACAGCAGAUACAGAGGCAGCUACUGGAGAACCGUGAGAGCCUGCCUGGGCUGCCCUCUG |
| | AGAAGAGGCGCCCUGCUGCUGCUGAGCAUCUACUUCUACUACAGCCUGCCUAACGCCGUGGGCCCUCCUU |
| | UCACCUGGAUGCUGGCCCUGCUGGGCCUGAGCCAGGCCCUGAACAUCCUGCUGGGCCUGAAGGGCCUGG |
| | CCCCUGCCGAGAUCAGCGCCCUGUGCGAGAAGGGCAACUUCAACGUGGCCCACGGCCUGGCCUGGAGCUA |
| | CUACAUCGGCUACCUGAGACUGAUCCUGCCUGAGCUGCAGGCCAGAAUCAGAACCUACAACCAGCACUAC |
| | AACAACCUGCUGAGAGGCGCCGUGAGCCAGAGACUGUACAUCCUGCUGCCUCUGGACUGCGGCGUGCCU |
| | GACAACCUGAGCAUGGCCGACCCUAACAUCAGAUUCCUGGACAAGCUGCCUCAGCAGACCGGCGACCACGC |
| | CGGCAUCAAGGACAGAGUGUACAGCAACAGCAUCUACGAGCUGCUGGAGAACGGCCAGAGAGCCGGCACC |
| | UGCGUGCUGGAGUACGCCACCCCUCUGCAGACCCUGUUCGCCAUGAGCCAGUACAGCCAGGCCGGCUUCA |
| | GCAGAGAGGACAGACUGGAGCAGGCCAAGCUGUUCUGCAGAACCCUGGAGGACAUCCUGGCCGACGCCCC |
| | UGAGAGCCAGAACAACUGCAGACUGAUCGCCUACCAGGAGCCUGCCGACGACAGCAGCUUCAGCCUGAGC |
| | CAGGAGGUGCUGAGACACCUGAGACAGGAGGAGAAGGAGGAGGUGACCGUGGGCAGCCUGAAGACCAGC |
| | GCCGUGCCUAGCACCAGCACCAUGAGCCAGGAGCCUGAGCUGCUGAUCAGCGGCAUGGAGAAGCCUCUGC |
| | CUCUGAGAACCGACUUCAGC |
| | (Hu STING(V147L); no epitope tag; nucleotide sequence) |
| 1447 | AUGCCUCACAGCAGCCUGCACCCUAGCAUCCCUUGCCCUAGAGGCCACGGCGCCCAGAAGGCCGCCCUGGU |
| | GCUGCUGAGCGCCUGCCUGGUGACCCUGUGGGGCCUGGGCGAGCCUCCUGAGCACACCCUGAGAUACCU |
| | GGUGCUGCACCUGGCCAGCCUGCAGCUGGGCCUGCUGCUGAACGGCGUGUGCAGCCUGGCCGAGGAGCU |
| | GAGACACAUCCACAGCAGAUACAGAGGCAGCUACUGGAGAACCGUGAGAGCCUGCCUGGGCUGCCCUCUG |
| | AGAAGAGGCGCCCUGCUGCUGCUGAGCAUCUACUUCUACUACAGCCUGCCUAACGCCGUGGGCCCUCCUU |
| | UCACCUGGAUGCUGGCCCUGCUGGGCCUGAGCCAGGCCCUGAACAUCCUGCUGGGCCUGAAGGGCCUGG |
| | CCCCUGCCGAGAUCAGCGCCGUGUGCGAGAAGGGCAACUUCAACGUGGCCCACGGCCUGGCCUGGAGCUA |
| | CUACAUCGGCUACCUGAGACUGAUCCUGCCUGAGCUGCAGGCCAGAAUCAGAACCUACAACCAGCACUAC |
| | AACAACCUGCUGAGAGGCGCCGUGAGCCAGAGACUGUACAUCCUGCUGCCUCUGGACUGCGGCGUGCCU |
| | GACAACCUGAGCAUGGCCGACCCUAACAUCAGAUUCCUGGACAAGCUGCCUCAGCAGACCGGCGACCACGC |
| | CGGCAUCAAGGACAGAGUGUACAGCAACAGCAUCUACGAGCUGCUGGAGAACGGCCAGAGAGCCGGCACC |
| | UGCGUGCUGGAGUACGCCACCCCUCUGCAGACCCUGUUCGCCAUGAGCCAGUACAGCCAGGCCGGCUUCA |
| | GCAGAGAGGACAGACUGGAGCAGGCCAAGCUGUUCUGCAGAACCCUGGAGGACAUCCUGGCCGACGCCCC |
| | UGAGAGCCAGAACAACUGCAGACUGAUCGCCUACCAGCAGCCUGCCGACGACAGCAGCUUCAGCCUGAGC |
| | CAGGAGGUGCUGAGACACCUGAGACAGGAGGAGAAGGAGGAGGUGACCGUGGGCAGCCUGAAGACCAGC |
| | GCCGUGCCUAGCACCAGCACCAUGAGCCAGGAGCCUGAGCUGCUGAUCAGCGGCAUGGAGAAGCCUCUGC |
| | CUCUGAGAACCGACUUCAGC |
| | (Hu STING (E315Q); no epitope tag; nucleotide sequence) |
| 1448 | AUGCCUCACAGCAGCCUGCACCCUAGCAUCCCUUGCCCUAGAGGCCACGGCGCCCAGAAGGCCGCCCUGGU |
| | GCUGCUGAGCGCCUGCCUGGUGACCCUGUGGGGCCUGGGCGAGCCUCCUGAGCACACCCUGAGAUACCU |
| | GGUGCUGCACCUGGCCAGCCUGCAGCUGGGCCUGCUGCUGAACGGCGUGUGCAGCCUGGCCGAGGAGCU |
| | GAGACACAUCCACAGCAGAUACAGAGGCAGCUACUGGAGAACCGUGAGAGCCUGCCUGGGCUGCCCUCUG |
| | AGAAGAGGCGCCCUGCUGCUGCUGAGCAUCUACUUCUACUACAGCCUGCCUAACGCCGUGGGCCCUCCUU |
| | UCACCUGGAUGCUGGCCCUGCUGGGCCUGAGCCAGGCCCUGAACAUCCUGCUGGGCCUGAAGGGCCUGG |
| | CCCCUGCCGAGAUCAGCGCCGUGUGCGAGAAGGGCAACUUCAACGUGGCCCACGGCCUGGCCUGGAGCUA |
| | CUACAUCGGCUACCUGAGACUGAUCCUGCCUGAGCUGCAGGCCAGAAUCAGAACCUACAACCAGCACUAC |
| | AACAACCUGCUGAGAGGCGCCGUGAGCCAGAGACUGUACAUCCUGCUGCCUCUGGACUGCGGCGUGCCU |
| | GACAACCUGAGCAUGGCCGACCCUAACAUCAGAUUCCUGGACAAGCUGCCUCAGCAGACCGGCGACCACGC |
| | CGGCAUCAAGGACAGAGUGUACAGCAACAGCAUCUACGAGCUGCUGGAGAACGGCCAGAGAGCCGGCACC |
| | UGCGUGCUGGAGUACGCCACCCCUCUGCAGACCCUGUUCGCCAUGAGCCAGUACAGCCAGGCCGGCUUCA |
| | GCAGAGAGGACAGACUGGAGCAGGCCAAGCUGUUCUGCAGAACCCUGGAGGACAUCCUGGCCGACGCCCC |
| | UGAGAGCCAGAACAACUGCAGACUGAUCGCCUACCAGGAGCCUGCCGACGACAGCAGCUUCAGCCUGAGC |
| | CAGGAGGUGCUGAGACACCUGAGACAGGAGGAGAAGGAGGAGGUGACCGUGGGCAGCCUGAAGACCAGC |
| | GCCGUGCCUAGCACCAGCACCAUGAGCCAGGAGCCUGAGCUGCUGAUCAGCGGCAUGGAGAAGCCUCUGC |
| | CUCUGGCCACCGACUUCAGC |
| | (Hu STING (R375A); no epitope tag; nucleotide sequence) |
| 1449 | AUGCCUCACAGCAGCCUGCACCCUAGCAUCCCUUGCCCUAGAGGCCACGGCGCCCAGAAGGCCGCCCUGGU |
| | GCUGCUGAGCGCCUGCCUGGUGACCCUGUGGGGCCUGGGCGAGCCUCCUGAGCACACCCUGAGAUACCU |
| | GGUGCUGCACCUGGCCAGCCUGCAGCUGGGCCUGCUGCUGAACGGCGUGUGCAGCCUGGCCGAGGAGCU |
| | GAGACACAUCCACAGCAGAUACAGAGGCAGCUACUGGAGAACCGUGAGAGCCUGCCUGGGCUGCCCUCUG |
| | AGAAGAGGCGCCCUGCUGCUGCUGAGCAUCUACUUCUACUACAGCCUGCCUAACGCCGUGGGCCCUCCUU |
| | UCACCUGGAUGCUGGCCCUGCUGGGCCUGAGCCAGGCCCUGAACAUCCUGCUGGGCCUGAAGGGCCUGG |
| | CCCCUGCCGAGAUCAGCGCCCUGUGCGAGAAGGGCAACUUCAGCAUGGCCCACGGCCUGGCCUGGAGCUA |
| | CUACAUCGGCUACCUGAGACUGAUCCUGCCUGAGCUGCAGGCCAGAAUCAGAACCUACAACCAGCACUAC |
| | AACAACCUGCUGAGAGGCGCCGUGAGCCAGAGACUGUACAUCCUGCUGCCUCUGGACUGCGGCGUGCCU |
| | GACAACCUGAGCAUGGCCGACCCUAACAUCAGAUUCCUGGACAAGCUGCCUCAGCAGACCGGCGACCACGC |
| | CGGCAUCAAGGACAGAGUGUACAGCAACAGCAUCUACGAGCUGCUGGAGAACGGCCAGAGAGCCGGCACC |
| | UGCGUGCUGGAGUACGCCACCCCUCUGCAGACCCUGUUCGCCAUGAGCCAGUACAGCCAGGCCGGCUUCA |
| | GCAGAGAGGACAGACUGGAGCAGGCCAAGCUGUUCUGCAGAACCCUGGAGGACAUCCUGGCCGACGCCCC |
| | UGAGAGCCAGAACAACUGCAGACUGAUCGCCUACCAGGAGCCUGCCGACGACAGCAGCUUCAGCCUGAGC |
| | CAGGAGGUGCUGAGACACCUGAGACAGGAGGAGAAGGAGGAGGUGACCGUGGGCAGCCUGAAGACCAGC |
| | GCCGUGCCUAGCACCAGCACCAUGAGCCAGGAGCCUGAGCUGCUGAUCAGCGGCAUGGAGAAGCCUCUGC |
| | CUCUGAGAACCGACUUCAGC |
| | (Hu SUING(V147L/N154S/V155M); no epiUope Uag; nucleoUide sequence) |
| 1450 | AUGCCUCACAGCAGCCUGCACCCUAGCAUCCCUUGCCCUAGAGGCCACGGCGCCCAGAAGGCCGCCCUGGU |
| | GCUGCUGAGCGCCUGCCUGGUGACCCUGUGGGGCCUGGGCGAGCCUCCUGAGCACACCCUGAGAUACCU |
| | GGUGCUGCACCUGGCCAGCCUGCAGCUGGGCCUGCUGCUGAACGGCGUGUGCAGCCUGGCCGAGGAGCU |
| | GAGACACAUCCACAGCAGAUACAGAGGCAGCUACUGGAGAACCGUGAGAGCCUGCCUGGGCUGCCCUCUG |
| | AGAAGAGGCGCCCUGCUGCUGCUGAGCAUCUACUUCUACUACAGCCUGCCUAACGCCGUGGGCCCUCCUU |
| | UCACCUGGAUGCUGGCCCUGCUGGGCCUGAGCCAGGCCCUGAACAUCCUGCUGGGCCUGAAGGGCCUGG |
| | CCCCUGCCGAGAUCAGCGCCCUGUGCGAGAAGGGCAACUUCAGCAUGGCCCACGGCCUGGCCUGGAGCUA |
| | CUACAUCGGCUACCUGAGACUGAUCCUGCCUGAGCUGCAGGCCAGAAUCAGAACCUACAACCAGCACUAC |
| | AACAACCUGCUGAGAGGCGCCGUGAGCCAGAGACUGUACAUCCUGCUGCCUCUGGACUGCGGCGUGCCU |
| | GACAACCUGAGCAUGGCCGACCCUAACAUCAGAUUCCUGGACAAGCUGCCUCAGCAGACCGGCGACCACGC |
| | CGGCAUCAAGGACAGAGUGUACAGCAACAGCAUCUACGAGCUGCUGGAGAACGGCCAGAGAGCCGGCACC |
| | UGCGUGCUGGAGUACGCCACCCCUCUGCAGACCCUGUUCGCCAUGAGCCAGUACAGCCAGGCCGGCUUCA |
| | GCAGAGAGGACAUGCUGGAGCAGGCCAAGCUGUUCUGCAGAACCCUGGAGGACAUCCUGGCCGACGCCCC |
| | UGAGAGCCAGAACAACUGCAGACUGAUCGCCUACCAGGAGCCUGCCGACGACAGCAGCUUCAGCCUGAGC |
| | CAGGAGGUGCUGAGACACCUGAGACAGGAGGAGAAGGAGGAGGUGACCGUGGGCAGCCUGAAGACCAGC |
| | GCCGUGCCUAGCACCAGCACCAUGAGCCAGGAGCCUGAGCUGCUGAUCAGCGGCAUGGAGAAGCCUCUGC |
| | CUCUGAGAACCGACUUCAGC |
| | (Hu STING(R284M/V147L/N154S/V155M); no epitope tag; nucleotide sequence) |
| 1451 | UGAUAAUAGGCUGGAGCCUCGGUGGCCUAGCUUCUUGCCCCUUGGGCCUCCCCCCAGCCCCUCCUCCCCU |
| | UCCUGCACCCGUACCCCCCAAACACCAUUGUCACACUCCAGUGGUCUUUGAAUAAAGUCUGAGUGGGCGG |
| | C |
| | (3′ UTR used in STING V155M construct, containing miR122 binding site) |
| 1452 | AUGGAGACCCCCAAGCCUAGAAUCCUGCCCUGGCUGGUGAGCCAGCUGGACCUGGGCCAGCUGGAGGGCG |
| | UAGCCUGGCUGGACGAGAGCAGAACCAGAUUCAGAAUCCCCUGGAAGCACGGCCUGAGACAAGACGCCCA |
| | GAUGGCCGACUUCGGCAUCUUCCAGGCCUGGGCCGAGGCCAGCGGCGCCUACACCCCUGGCAAGGAUAAG |
| | CCCGAUGUGAGCACCUGGAAGAGAAACUUCAGAAGCGCCCUGAACAGAAAGGAGGUGCUGAGACUGGCC |
| | GCCGACAAUAGCAAGGACCCCUACGACCCCCACAAGGUGUACGAGUUCGUUACCCCCGGCGCCAGGGACU |
| | UCGUGCACCUGGGCGCCAGCCCCGACACCAACGGCAAGAGCAGCCUGCCCCACAGCCAGGAGAACCUGCCC |
| | AAGCUGUUCGAUGGCCUGAUCCUGGGCCCCCUGAAGGACGAGGGCAGCAGCGACCUGGCCAUCGUGAGC |
| | GACCCUAGCCAGCAGCUGCCCUCCCCCAACGUGAACAACUUCCUGAACCCCGCCCCCCAGGAGAACCCCCUG |
| | AAGCAACUGCUGGCCGAGGAGCAGUGGGAGUUCGAGGUGACCGCCUUCUACAGAGGCAGACAGGUGUUC |
| | CAGCAGACCCUGUUCUGCCCCGGCGGCCUGAGACUGGUAGGCAGCACCGCUGACAUGACCCUGCCCUGGC |
| | AGCCCGUGACCCUGCCCGACCCCGAAGGCUUUCUGACCGACAAGCUGGUGAAGGAGUACGUCGGCCAAGU |
| | GCUGAAGGGCCUGGGCAACGGCCUGGCCCUGUGGCAGGCCGGCCAGUGCCUGUGGGCCCAGAGACUCGG |
| | CCACAGCCACGCCUUCUGGGCCCUGGGCGAGGAACUCCUGCCCGAUAGCGGCAGAGGCCCCGACGGCGAG |
| | GUGCACAAGGACAAGGACGGCGCCGUGUUCGACCUGCGCCCCUUCGUGGCCGACCUGAUCGCCUUCAUGG |
| | AGGGCAGCGGCCACAGCCCCAGAUAUACCCUGUGGUUCUGCAUGGGCGAGAUGUGGCCCCAGGACCAGCC |
| | CUGGGUGAAGAGACUGGUGAUGGUGAAGGUGGUGCCCACCUGCCUGAAAGAGCUGCUGGAGAUGGCCA |
| | GAGAGGGCGGCGCCAGCUCCCUGAAAACCGUGGACCUGCACAUUGACAACAGCCAGCCCAUCAGCCUGACC |
| | AGCGACCAGUACAAGGCCUACCUGCAGGACCUGGUGGAGGACAUGGACUUCCAGGCCACCGGCAACAUC |
| | (super mouse IRF3 S396D; no epitope tag) |
| 1453 | AUGGGCACCCCCAAGCCCAGAAUCCUGCCCUGGCUGGUGAGCCAGCUGGACCUGGGCCAGCUGGAGGGAG |
| | UGGCCUGGGUGAACAAGAGCAGAACCAGAUUCAGAAUCCCCUGGAAGCACGGCCUCAGACAGGACGCCCA |
| | GCAGGAGGACUUCGGCAUUUUUCAGGCUUGGGCCGAGGCCACCGGCGCCUACGUGCCCGGCAGAGACAA |
| | GCCCGACCUGCCCACCUGGAAAAGAAACUUCAGAAGCGCCUUGAAUAGAAAGGAGGGCCUGAGACUGGCC |
| | GAGGACAGAAGCAAGGACCCCCACGACCCUCACAAGAUCUACGAGUUCGUGAAUAGCGGCGUGGGCGACU |
| | UUAGCCAGCCCGACACCAGCCCCGACACCAACGGCGGCGGCAGCACCAGCGACACGCAGGAGGACAUCCUG |
| | GAUGAACUGCUGGGCAACAUGGUGCUGGCCCCCCUGCCCGAUCCCGGCCCCCCUUCGCUUGCCGUGGCCC |
| | CCGAGCCCUGCCCCCAGCCCCUGAGAAGCCCCUCUCUGGAUAACCCCACCCCCUUCCCCAACCUGGGCCCCA |
| | GCGAGAAUCCACUGAAGAGACUUCUGGUCCCCGGCGAGGAGUGGGAGUUCGAGGUGACCGCCUUCUACA |
| | GAGGCAGACAGGUGUUCCAGCAGACCAUCAGCUGCCCCGAAGGCCUGAGAUUAGUGGGCAGCGAAGUGG |
| | GCGACAGGACCCUGCCCGGGUGGCCCGUGACCCUGCCCGAUCCCGGCAUGAGCCUGACCGACAGAGGUGU |
| | GAUGAGCUACGUGAGACACGUGCUGAGCUGCCUGGGCGGCGGCCUGGCACUGUGGAGAGCCGGCCAGUG |
| | GCUGUGGGCCCAGAGACUGGGCCACUGCCACACCUACUGGGCCGUGAGCGAGGAGCUGCUGCCCAACAGC |
| | GGCCACGGCCCCGACGGCGAGGUGCCCAAGGACAAGGAAGGGGGCGUGUUCGACCUGGGCCCCUUCAUCG |
| | UAGACCUGAUCACCUUUACCGAGGGCAGCGGCAGGAGCCCCAGAUACGCCCUGUGGUUCUGCGUGGGCG |
| | AAAGCUGGCCCCAGGACCAGCCCUGGACCAAGAGACUGGUGAUGGUGAAGGUAGUGCCCACCUGCCUGAG |
| | AGCCUUAGUGGAGAUGGCCAGAGUGGGCGGGGCCAGCAGCCUGGAGAACACCGUGGAUCUUCACAUCGA |
| | CAACAGCCACCCCCUGAGCCUGACCAGCGACCAGUACAAGGCCUACCUGCAGGACCUGGUGGAGGGCAUG |
| | GACUUCCAGGGCCCCGGCGAGACC |
| | (super human IRF3 S396D; no epitope tag) |
| 1454 | AUGGCGCUGGCCCCCGAAAGAGCCGCCCCCAGAGUCCUCUUCGGCGAAUGGCUCCUUGGCGAAAUUUCGU |
| | CGGGCUGCUACGAGGGCUUACAAUGGCUGGAUGAGGCGAGAACCUGUUUCAGGGUGCCCUGGAAACACU |
| | UCGCCAGAAAGGAUCUAAGCGAAGCAGAUGCUAGAAUUUUUAAGGCUUGGGCCGUGGCCAGGGGAAGA |
| | UGGCCCCCCUCGAGCAGAGGCGGCGGCCCUCCCCCCGAGGCAGAAACGGCCGAGAGAGCCGGAUGGAAAA |
| | CCAAUUUCAGAUGCGCCCUGAGAUCUACAAGAAGAUUCGUGAUGCUUAGAGACAACAGCGGAGAUCCCG |
| | CCGAUCCCCAUAAGGUGUAUGCCCUGUCCCGGGAGCUGUGCUGGAGGGAAGGGCCUGGCACUGACCAGA |
| | CCGAAGCCGAAGCCCCCGCGGCCGUGCCGCCGCCCCAAGGAGGCCCACCAGGCCCUUUCCUCGCUCACACCC |
| | ACGCCGGUCUGCAAGCCCCGGGACCUCUACCUGCCCCUGCCGGCGAUAAAGGCGACCUGUUGCUGCAGGC |
| | CGUCCAACAGAGCUGCCUGGCCGAUCAUCUGCUCACAGCCAGCUGGGGCGCUGACCCCGUCCCAACAAAG |
| | GCCCCCGGUGAGGGCCAAGAAGGCCUGCCUCUGACCGGCGCCUGUGCCGGCGGCCCUGGCCUGCCUGCUG |
| | GCGAGCUGUACGGAUGGGCUGUCGAAACCACUCCCUCCCCCGGCCCCCAACCUGCGGCCCUGACAACCGGC |
| | GAGGCAGCCGCACCCGAAAGCCCCCACCAGGCCGAACCCUACCUCAGUCCCAGCCCCUCCGCCUGCACCGCU |
| | GUGCAGGAGCCCAGCCCCGGUGCUCUGGACGUAACAAUCAUGUACAAAGGCAGAACCGUGCUUCAGAAGG |
| | UGGUUGGACACCCCUCCUGUACUUUUCUCUACGGCCCCCCCGACCCUGCCGUGAGAGCUACCGACCCGCA |
| | ACAGGUGGCCUUUCCCUCGCCCGCCGAACUGCCCGAUCAAAAACAGCUGAGAUACACCGAGGAGCUGCUG |
| | AGACACGUGGCGCCGGGCUUACACCUAGAGUUGAGAGGCCCCCAACUCUGGGCCAGACGCAUGGGCAAGU |
| | GUAAGGUGUACUGGGAGGUCGGGGGCCCUCCCGGCUCUGCCAGCCCCAGCACCCCUGCUUGUCUCUUGCC |
| | CAGAAACUGUGAUACCCCCAUCUUCGACUUCCGUGUAUUUUUCCAGGAACUGGUCGAGUUUAGAGCCAG |
| | ACAGAGACGAGGCAGCCCCAGAUAUACAAUCUACCUCGGCUUCGGCCAGGACCUGAGUGCCGGCAGACCU |
| | AAGGAGAAGUCGCUGGUCCUAGUGAAGUUAGAGCCCUGGCUAUGUAGAGUGCACCUGGAGGGCACCCAG |
| | AGAGAAGGAGUGAGCAGCCUGGACAGCAGCAGCCUGAGUCUGUGCCUGAGCUCCGCCAACUCGCUGUAU |
| | GAUGACAUCGAGUGUUUCCUCAUGGAGCUGGAGCAGCCCGCC |
| | (Wild-type Hu IRF7 isoform A; P037 without epitope tag) |
| 1455 | AUGGCCCUUGCCCCUGAGCGGGCCGCCCCCAGAGUGUUAUUCGGCGAGUGGCUGCUGGGCGAGAUCAGC |
| | AGCGGCUGCUACGAGGGACUGCAGUGGCUGGACGAGGCUAGAACCUGCUUCAGAGUGCCCUGGAAGCAU |
| | UUCGCCAGAAAAGACCUGAGCGAGGCUGAUGCUAGAAUCUUCAAAGCCUGGGCUGUGGCCCGAGGAAGA |
| | UGGCCCCCCAGCAGCAGAGGAGGCGGCCCUCCUCCCGAGGCCGAAACCGCAGAGCGUGCUGGCUGGAAAA |
| | CCAACUUUAGGUGUGCCCUGAGGAGCACCAGAAGAUUCGUUAUGCUCAGAGACAACAGCGGGGACCCCGC |
| | CGACCCGCACAAGGUGUACGCCUUAAGUAGGGAGCUGUGCUGGAGAGAGGGACCGGGGACCGACCAAAC |
| | CGAGGCUGAGGCGCCCGCCGCCGUUCCACCUCCCCAGGGUGGUCCCCCAGGGCCCUUUCUGGCACACACCC |
| | ACGCCGGAUUACAGGCGCCAGGGCCCUUACCCGCCCCCGCCGGAGACAAAGGCGACCUCCUGCUGCAAGCC |
| | GUGCAACAAAGCUGCCUGGCCGAUCACUUACUAACCGCUAGCUGGGGCGCCGAUCCUGUUCCCACCAAGG |
| | CCCCCGGUGAAGGGCAAGAAGGACUGCCCUUAACCGGCGCCUGUGCCGGAGGCCCUGGUCUGCCAGCCGG |
| | CGAGCUGUACGGUUGGGCUGUCGAAACAACACCCAGUCCGGGCCCACAGCCUGCCGCUCUGACCACCGGC |
| | GAAGCCGCCGCCCCCGAGAGCCCACACCAGGCUGAACCCUACCUGAGCCCCAGCCCCAGCGCCUGCACCGCU |
| | GUGCAGGAGCCUAGCCCCGGCGCUCUUGAUGUGACAAUAAUGUACAAGGGCAGGACCGUGCUGCAAAAG |
| | GUCGUGGGCCAUCCGUCGUGUACCUUUCUGUACGGCCCUCCAGACCCCGCGGUUAGAGCCACCGACCCCC |
| | AGCAAGUCGCCUUCCCCUCCCCCGCCGAACUGCCCGACCAAAAGCAGCUGCGGUACACAGAAGAACUACUU |
| | AGACACGUGGCCCCCGGUCUGCACUUGGAGCUGAGAGGCCCCCAGCUCUGGGCCAGAAGAAUGGGCAAGU |
| | GCAAAGUGUACUGGGAGGUGGGCGGCCCACCCGGCUCAGCUUCGCCCUCCACACCCGCAUGCCUGCUGCC |
| | CAGAAAUUGCGACACGCCCAUCUUCGAUUUUAGAGUGUUCUUUCAGGAGUUGGUGGAGUUCAGAGCCA |
| | GACAAAGACGCGGCAGCCCCAGAUACACCAUUUACCUCGGCUUCGGCCAGGACCUCAGCGCUGGCAGACCC |
| | AAGGAGAAGAGUCUGGUCCUCGUGAAGCUGGAGCCCUGGCUGUGCAGAGUGCACCUGGAGGGCACCCAG |
| | CGUGAAGGCGUGAGCAGCCUGGAUUCAAGCGACCUGGACCUAUGCCUAAGCAGCGCUAACUCACUGUAC |
| | GACGAUAUCGAAUGCUUCCUGAUGGAACUGGAGCAGCCUGCC |
| | (constitutively active Hu IRF7 S477D/S479D; P033 without epitope tag) |
| 1456 | AUGGCCCUGGCACCCGAGAGGGCCGCCCCCAGGGUGCUCUUCGGCGAGUGGUUACUAGGCGAAAUUAGC |
| | AGCGGCUGCUAUGAAGGCCUUCAGUGGCUGGACGAGGCCAGAACCUGCUUUAGAGUUCCCUGGAAGCAC |
| | UUCGCCCGGAAAGAUCUCUCUGAAGCCGACGCCAGAAUAUUCAAGGCCUGGGCUGUCGCCAGGGGCAGG |
| | UGGCCACCCUCCAGCCGAGGUGGCGGCCCUCCCCCUGAGGCUGAGACUGCGGAAAGGGCGGGCUGGAAGA |
| | CCAAUUUCAGAUGCGCUCUGAGAAGCACCAGACGUUUUGUGAUGCUAAGAGACAAUAGCGGCGAUCCCG |
| | CCGACCCCCAUAAGGUAUACGCACUGAGCCGAGAGCUCUGUUGGAGAGAAGGCCCCGGCACCGACCAGAC |
| | CGAGGCUGAAGCCCCUGCAGCCGUGCCCCCCCCUCAAGGCGGGCCCCCCGGCCCCUUCCUGGCCCAUACCCA |
| | UGCAGGGUUACAAGCACCCGGGCCCUUGCCCGCCCCAGCGGGAGACAAGGGCGACCUCUUACUGCAGGCC |
| | GUGCAACAAAGUUGUCUGGCGGACCACCUGCUGACCGCAUCAUGGGGCGCGGAUCCUGUGCCCACCAAGG |
| | CACCCGGCGAAGGCCAGGAGGGCCUGCCCUUGACCGGCGCCUGCGCUGGCGGACCCGGCCUACCUGCUGG |
| | CGAACUGUAUGGCUGGGCCGUAGAGACGACUCCCAGCCCUGGCCCACAACCCGCGGCUUUGACCACCGGC |
| | GAAGCCGCCGCCCCCGAGUCUCCGCACCAGGCCGAGCCUUACCUCAGCCCAAGCCCUAGCGCCUGCACCGCC |
| | GUGCAAGAACCUAGCCCCGGAGCCCUGGAUGUGACAAUCAUGUACAAGGGUAGAACCGUACUGCAAAAG |
| | GUGGUGGGUCAUCCCAGCUGCACCUUUCUUUACGGCCCACCCGACCCUGCCGUGCGAGCCACAGACCCAC |
| | AACAGGUCGCCUUCCCAAGCCCCGCCGAACUGCCCGAUCAGAAACAGCUGAGAUAUACAGAGGAGCUUCU |
| | GCGGCACGUAGCUCCCGGCCUACAUCUCGAGCUGAGGGGCCCACAACUGUGGGCCAGACGCAUGGGCAAA |
| | UGCAAGGUCUACUGGGAAGUGGGAGGCCCCCCCGGCAGCGCAUCUCCCAGCACGCCCGCGUGCCUGCUGC |
| | CUAGAAAUUGCGACACCCCCAUCUUUGACUUCCGGGUAUUCUUUCAGGAGCUGGUAGAGUUCAGAGCCA |
| | GGCAGCGGAGGGGCUCCCCCAGAUACACAAUCUACCUGGGCUUCGGACAGGACCUGUCCGCCGGCCGCCC |
| | CAAGGAAAAGAGCCUGGUGCUGGUGAAGCUGGAGCCCUGGCUGUGUAGGGUACACCUCGAAGGCACCCA |
| | GAGAGAAGGAGUGAGCUCGCUUGAUGACAGCGAUCUGUCGGAUUGCCUUAGCAGCGCCAACAGCCUGUA |
| | UGAUGAUAUCGAGUGCUUCCUUAUGGAACUGGAGCAGCCCGCC |
| | (constitutively active Hu IRF7 S475D/S477D/L480D; P034 without epitope tag) |
| 1457 | AUGGCCCUAGCCCCCGAAAGAGCAGCUCCCAGAGUGCUGUUCGGCGAAUGGCUGCUUGGCGAGAUCAGC |
| | AGCGGCUGCUACGAAGGCCUGCAGUGGCUGGACGAAGCCCGCACCUGUUUCAGAGUGCCCUGGAAGCAC |
| | UUCGCUAGAAAGGAUUUGAGCGAGGCUGAUGCUAGAAUCUUUAAGGCUUGGGCUGUGGCAAGAGGCAG |
| | AUGGCCGCCUAGUAGCAGAGGGGGCGGACCUCCCCCCGAGGCUGAGACCGCUGAGAGAGCAGGGUGGAA |
| | AACCAACUUCAGAUGCGCGCUGAGAAGCACCCGAAGAUUCGUGAUGCUACGUGACAAUAGCGGCGACCCC |
| | GCCGACCCCCACAAAGUGUACGCCCUGUCCCGAGAACUUUGCUGGAGAGAGGGACCCGGCACCGAUCAAA |
| | CAGAGGCUGAGGCCCCGGCCGCUGUACCCCCGCCCCAAGGAGGCCCCCCAGGCCCCUUUCUGGCUCAUACA |
| | CAUGCCGGCCUGCAGGCACCCGGGCCCCUCCCGGCUCCUGCCGGCGACAAGGGCGAUCUCCUUCUCCAGGC |
| | CGUGCAGCAGAGCUGCCUGGCCGAUCACCUGCUGACCGCCUCGUGGGGCGCCGACCCCGUGCCCACCAAA |
| | GCCCCGGGUGAAGGCCAAGAGGGGCUCCCUUUAACCGGAGCAUGCGCCGGAGGCCCCGGCCUGCCAGCCG |
| | GCGAGUUAUAUGGCUGGGCUGUGGAGACCACACCCUCCCCCGGCCCUCAACCCGCUGCCCUGACCACCGG |
| | UGAGGCCGCCGCCCCCGAGAGCCCACACCAGGCCGAACCCUACCUGAGCCCUAGCCCUAGCGCCUGCACCGC |
| | CGUGCAAGAACCCAGCCCCGGAGCCCUGGAUGUGACCAUUAUGUACAAGGGCCGGACAGUGCUGCAAAAG |
| | GUUGUGGGACACCCGAGCUGCACCUUUCUGUACGGUCCGCCUGACCCCGCCGUGAGAGCCACGGACCCGC |
| | AGCAGGUGGCCUUCCCCUCACCCGCGGAGCUGCCCGACCAAAAGCAACUCAGAUACACAGAAGAACUAUU |
| | GCGUCACGUCGCGCCCGGCCUGCAUCUGGAGCUGAGAGGCCCCCAGCUCUGGGCCAGAAGGAUGGGCAAA |
| | UGCAAGGUGUACUGGGAGGUGGGAGGCCCCCCCGGCAGCGCCAGCCCCAGCACUCCCGCGUGCCUGCUGC |
| | CCAGAAAUUGCGACACUCCCAUCUUCGAUUUCAGGGUGUUCUUCCAGGAGCUGGUGGAGUUCAGAGCCA |
| | GGCAGAGAAGGGGUAGCCCCAGAUACACAAUCUAUCUAGGCUUUGGACAAGAUCUGAGCGCCGGCCGGC |
| | CUAAGGAAAAAAGCCUGGUGCUGGUAAAGCUGGAGCCGUGGCUUUGUAGAGUGCACCUGGAGGGGACG |
| | CAGCGAGAGGGCGUGAGCAGCUUAGACGACGAUGACUUGGAUCUGUGUCUCGACAGCGCCAACGACUUG |
| | UACGACGACAUCGAGUGCUUCCUGAUGGAACUGGAGCAGCCCGCC |
| | (constitutively active Hu IRF7 S475D/S476D/S477D/S479D/S483D/S487D; P035 without |
| | epitope tag) |
| 1458 | AUGGCCCUGGCCCCCGAGAGAGCCGCCCCCAGAGUGCUCUUCGGCGAGUGGCUGCUGGGCGAGAUAAGCA |
| | GCGGCUGCUACGAAGGUCUGCAGUGGCUAGACGAGGCCAGAACCUGCUUUAGAGUGCCCUGGAAGCACU |
| | UCGCUCGAAAGGACCUGUCCGAGGCCGAUGCUAGAAUUUUUAAGGCUUGGGCCGUCGCUAGGGGAAGA |
| | UGGCCCCCUAGCAGUAGAGGCGGCGGCCCCCCUCCCGAAGCCGAGACGGCCGAGAGGGCCGGCUGGAAAA |
| | CCAAUUUCAGAUGCGCCCUGAGGAGCACCCGCAGGUUCGUAAUGCUGCGAGACAAUAGCGGCGAUCCUGC |
| | GGAUCCUCACAAGGUUUACGCCUUGAGUAGAGAACUGUGCUGGCGGGAGGGCCCCGGAACCGACCAGAC |
| | GGAGGCAGAGGCACCCGCUGCCGUGCCCCCCCCUCAAGGAGGACCCCCUGGACCCUUUCUGGCCCACACCC |
| | ACGCUGGUCUGCAGGCCCCAGGCCCACUGCCCGCCCCAGCGGGCGAUAAGGGUGACCUGCUCCUACAGGC |
| | GGUGCAACAGAGCUGUCUGGCCGACCACCUGUUGACCGCCAGCUGGGGGGCCGACCCGGUGCCCACCAAA |
| | GCUCCCGGAGAGGGCCAAGAAGGCCUCCCACUAACUGGCGCCUGCGCCGGGGGCCCGGGAUUACCCGCCG |
| | GCGAGCUGUAUGGCUGGGCCGUGGAGACCACGCCCAGCCCCGAGGGCGUGUCGUCCCUGGACAGCAGCAG |
| | CCUGAGCCUGUGCCUGAGCUCCGCCAACAGCCUGUAUGACGACAUCGAGUGCUUCCUGAUGGAGCUGGA |
| | ACAACCCGCC |
| | (constitutively active truncated Hu IRF7 1-246 + 468-503; P032 without epitope tag) |
| 1459 | AUGGCACUGGCGCCUGAAAGAGCCGCUCCGCGUGUGCUCUUCGGCGAGUGGCUGCUGGGCGAGAUCAGC |
| | UCCGGCUGCUACGAGGGUCUACAGUGGCUGGACGAGGCCAGAACCUGUUUUAGAGUGCCCUGGAAGCAC |
| | UUCGCGAGAAAGGACCUGAGCGAGGCCGACGCCAGAAUCUUCAAAGCCUGGGCAGUGGCUAGGGGCAGA |
| | UGGCCUCCCAGCAGCCGGGGCGGCGGCCCACCCCCCGAGGCCGAAACCGCCGAAAGAGCUGGCUGGAAGAC |
| | CAACUUCAGAUGCGCCCUGAGAAGCACCAGAAGAUUUGUCAUGCUGAGAGAUAAUUCAGGAGACCCCGCC |
| | GACCCUCACAAGGUGUACGCCCUGUCCAGAGAGCUGUGUUGGAGAGAGGGCCCCGGAACCGACCAGACCG |
| | AGGCCGAGGCUCCAGCUGCCGUGCCACCCCCCCAAGGCGGACCACCCGGCCCCUUCUUGGCACAUACGCAC |
| | GCCGGCCUCCAGGCUCCCGGCCCUCUGCCCGCCCCUGCUGGUGACAAAGGCGAUCUGCUGCUGCAAGCCG |
| | UCCAGCAAUCCUGCUUGGCUGACCACCUGCUGACCGCUAGCUGGGGAGCCGACCCCGUUCCCACCAAGGC |
| | UCCCGGAGAAGGACAGGAGGGCCUGCCCCUUACCGGCGCUUGCGCGGGGGGCCCUGGCUUGCCUGCCGG |
| | CGAACUGUACGGCUGGGCCGUGGAGACCACGCCUUCCCCCGAGGGCGUGUCCAGCCUGGACGAUGAUGAC |
| | CUGGAUCUGUGCCUGGACAGCGCCAACGACCUGUACGAUGACAUCGAGUGCUUUUUGAUGGAGCUGGAG |
| | CAGCCCGCC |
| | (constitutively active truncated Hu IRF7 1-246 + 468-503 plus |
| | S475D/S476D/S477D/S479D/S483D/S487D; P036 without epitope tag) |
| 1460 | AUGGCCCUGGCCCCCGAGAGAGCCGCGCCCAGAGUGCUGUUCGGCGAAUGGCUGCUGGGCGAGAUCAGC |
| | AGCGGCUGCUAUGAGGGCCUGCAGUGGCUCGACGAAGCCAGGACGUGCUUCAGAGUCCCCUGGAAGCAC |
| | UUCGCCAGAAAGGAUCUGAGCGAGGCUGACGCCAGAAUCUUCAAGGCCUGGGCAGUUGCGCGUGGGAGA |
| | UGGCCCCCCAGCUCGCGGGGCGGCGGUCCCCCCCCUGAGGCCGAGACCGCCGAAAGAGCCGGAUGGAAAAC |
| | CAACUUUCGAUGCGCCCUCAGAAGCACCAGACGGUUUGUGAUGCUGAGAGAUAACAGCGGCGACCCUGCA |
| | GACCCCCAUAAAGUGUAUGCCCUGAGCAGAGAGCUGUGUUGGCGAGAGGGCCCCGGAACCGACCAAACCG |
| | AGGCCGAGGCCCCCGCCGCCGUACCCCCCCCUCAAGGCCCCCAGCCUGCUGCUCUGACCACGGGAGAAGCC |
| | GCCGCUCCUGAGAGCCCCCACCAAGCCGAGCCCUAUCUGAGCCCUAGCCCCAGCGCCUGCACCGCCGUGCA |
| | GGAGCCCUCACCGGGCGCCCUAGACGUGACCAUCAUGUACAAGGGGCGCACGGUGCUGCAAAAGGUGGU |
| | GGGCCACCCCAGCUGCACCUUCCUGUACGGCCCCCCCGACCCUGCCGUGAGAGCCACCGACCCCCAGCAAG |
| | UCGCCUUCCCCAGCCCCGCCGAGCUGCCCGACCAGAAGCAGCUGAGGUACACCGAGGAGUUGCUGAGACA |
| | UGUGGCCCCCGGCUUGCACCUCGAGCUGAGAGGCCCGCAGCUCUGGGCCAGAAGAAUGGGCAAGUGCAA |
| | GGUGUACUGGGAGGUGGGCGGCCCCCCCGGCAGCGCGAGCCCAAGCACCCCGGCCUGCCUGCUGCCUAGA |
| | AACUGCGACACCCCUAUCUUCGACUUCAGAGUAUUUUUCCAGGAGCUGGUCGAGUUCAGGGCCAGACAG |
| | CGUAGAGGCAGCCCCAGAUACACCAUCUACCUUGGAUUCGGCCAGGACCUGAGCGCCGGCAGACCCAAAG |
| | AGAAGUCCCUGGUACUGGUGAAGCUAGAGCCCUGGCUGUGUAGGGUGCAUCUGGAAGGCACCCAAAGAG |
| | AGGGCGUAAGCUCGCUUGACAGCAGCAGCCUCAGCCUGUGCCUGAGCAGCGCUAACAGCUUAUACGACGA |
| | CAUCGAGUGCUUCCUGAUGGAGCUGGAACAACCCGCC |
| | (truncated Hu IRF7 1-151 + 247-503; P038 without epitope tag; null mutation) |
| 1461 | AUGGGCGGCCCUCCCGGGCCUUUCCUGGCCCAUACACACGCCGGCCUACAGGCUCCUGGCCCUCUGCCCGC |
| | CCCGGCCGGCGACAAGGGCGACCUCCUGCUGCAGGCCGUGCAGCAGUCCUGUCUGGCCGACCACCUGCUG |
| | ACUGCUAGCUGGGGCGCCGAUCCCGUGCCCACCAAGGCCCCAGGAGAGGGGCAAGAGGGCCUGCCUCUAA |
| | CCGGCGCAUGCGCAGGUGGACCAGGCCUCCCCGCCGGCGAGCUGUAUGGUUGGGCCGUGGAGACAACCCC |
| | CAGCCCCGGCCCGCAGCCUGCUGCGCUGACCACAGGCGAGGCCGCUGCCCCUGAGAGCCCCCACCAAGCUG |
| | AACCCUACCUGAGCCCCAGCCCCUCUGCCUGCACAGCGGUGCAGGAGCCCAGUCCCGGCGCCUUGGACGUG |
| | ACCAUCAUGUAUAAGGGCAGGACUGUGUUACAAAAGGUAGUGGGCCACCCAAGUUGUACCUUUCUGUAC |
| | GGGCCCCCCGACCCAGCCGUGCGCGCCACCGACCCCCAGCAGGUGGCCUUCCCCAGCCCCGCUGAGUUGCC |
| | CGAUCAGAAACAACUCCGGUACACCGAGGAAUUACUUAGACAUGUGGCUCCCGGCCUGCAUCUGGAGCUU |
| | AGAGGUCCACAGUUGUGGGCCAGAAGAAUGGGCAAGUGCAAGGUUUAUUGGGAGGUCGGAGGCCCCCC |
| | GGGCAGCGCCAGCCCCAGCACCCCCGCCUGUCUUCUGCCCAGAAACUGCGACACCCCAAUCUUCGAUUUCA |
| | GAGUGUUUUUCCAGGAACUGGUGGAGUUCAGAGCAAGGCAAAGAAGAGGCAGCCCUAGAUACACCAUCU |
| | ACCUGGGCUUUGGCCAAGACCUGAGCGCCGGCAGACCCAAGGAAAAAUCCCUGGUCCUGGUGAAACUGGA |
| | GCCCUGGCUGUGCAGAGUCCACCUGGAGGGCACCCAGAGAGAGGGCGUGAGCAGCCUGGACUCGAGCAG |
| | CCUGUCCCUGUGUCUGAGCAGCGCGAAUUCGCUAUAUGACGACAUCGAAUGCUUUCUGAUGGAGCUGGA |
| | ACAGCCCGCC |
| | (truncated Hu IRF7 152-503; P039 without epitope tag; null mutation) |
| 1462 | AUGCCUCACAGCAGCCUCCACCCUAGCAUCCCUUGCCCUAGAGGCCACGGCGCCCAGAAGGCCGCCCUCGU |
| | GCUUUUAAGCGCCUGCUUGGUGACCCUUUGGGGCUUGGGCGAGCCUCCAGAGCACACCUUGAGAUAUUU |
| | GGUGCUCCACCUGGCCAGCCUUCAGCUGGGCUUGUUACUCAACGGCGUGUGCAGCCUGGCCGAGGAGCU |
| | GAGACACAUCCACAGCAGAUACAGAGGCAGCUACUGGAGAACCGUGAGAGCGUGUCUGGGCUGCCCUCU |
| | GAGAAGAGGCGCCUUGCUUCUUCUCAGUAUCUACUUCUACUACUCCCUGCCUAACGCCGUGGGCCCUCCU |
| | UUCACCUGGAUGCUGGCACUGCUCGGCCUCAGCCAGGCCCUGAACAUCUUGUUGGGCUUGAAGGGCCUG |
| | GCCCCUGCCGAGAUCAGCGCCGUGUGCGAGAAGGGCAACUUCAACAUGGCCCACGGAUUGGCUUGGAGC |
| | UACUACAUCGGCUACCUGAGACUGAUCCUGCCUGAGCUGCAGGCCAGAAUCAGAACCUACAACCAGCACU |
| | ACAACAACCUGCUGCGCGGCGCAGUGAGCCAGAGACUGUAUAUUCUGCUGCCUCUGGACUGCGGCGUGC |
| | CUGACAACCUGAGCAUGGCCGACCCUAACAUCAGAUUCCUGGACAAGCUGCCUCAGCAGACCGGCGACCAC |
| | GCCGGCAUCAAGGACAGAGUGUACAGCAACAGCAUCUAUGAGCUGCUCGAGAAUGGCCAGAGAGCCGGCA |
| | CCUGCGUGCUGGAGUACGCCACCCCUCUGCAGACCCUGUUCGCCAUGAGCCAGUAUAGUCAAGCUGGCUU |
| | CAGCAGAGAGGACAGACUGGAGCAGGCCAAGCUGUUCUGCAGAACCCUGGAGGACAUUCUGGCUGACGC |
| | CCCUGAGAGCCAGAACAACUGCCGACUGAUCGCCUACCAGGAACCAGCCGACGACAGCAGCUUCAGUCUU |
| | UCUCAGGAGGUUCUUCGCCACUUGCGCCAGGAGGAGAAGGAGGAGGUGACCGUGGGCAGCCUGAAGACC |
| | UCCGCAGUCCCUAGCACCAGCACCAUGAGUCAGGAGCCGGAGCUAUUAAUCAGCGGCAUGGAGAAGCCUC |
| | UUCCACUCCGAACCGACUUCAGCGCCACCAACUUCAGCCUGCUGAAGCAGGCAGGUGACGUUGAGGAGAA |
| | UCCGGGACCUAUGACCGAGUACAAGCUGGUGGUUGUGGGCGCCGACGGCGUGGGCAAGAGCGCCCUGAC |
| | CAUCCAGCUGAUCCAG |
| | (KRAS(G12D)25 mer_nt.STING(V155M)) |
| 1463 | AUGACCGAGUACAAGCUAGUAGUCGUGGGCGCCGACGGCGUGGGCAAGAGCGCCCUCACCAUCCAGCUAA |
| | UCCAGGCCACCAACUUCAGCUUGCUCAAGCAGGCCGGCGACGUGGAGGAGAACCCAGGCCCUAUGCCUCA |
| | CAGCAGCCUUCACCCUAGCAUCCCUUGCCCUAGAGGCCACGGCGCCCAGAAGGCCGCCCUGGUGCUGCUG |
| | AGCGCCUGCCUGGUGACCCUGUGGGGCCUGGGCGAGCCUCCUGAGCACACCCUGAGAUAUCUGGUGCUU |
| | CACCUGGCCAGUUUACAGCUGGGCCUGCUUCUUAACGGCGUGUGCAGCCUGGCCGAGGAGCUGAGACAC |
| | AUCCACAGCAGAUACAGAGGCAGCUACUGGAGAACCGUGAGAGCCUGCCUAGGCUGCCCUCUGAGAAGAG |
| | GCGCUCUGUUGCUACUUUCCAUCUACUUCUACUACUCCCUGCCUAACGCCGUGGGCCCUCCUUUCACUUG |
| | GAUGCUGGCGUUGCUGGGUCUGAGCCAGGCCCUGAACAUCCUUCUCGGUCUGAAGGGCCUGGCCCCUGC |
| | CGAGAUCAGCGCCGUGUGCGAGAAGGGCAACUUCAACAUGGCCCACGGACUCGCCUGGAGCUACUACAUC |
| | GGCUACCUGAGACUGAUCCUGCCUGAGCUGCAGGCCAGAAUCAGAACCUACAACCAGCACUACAACAACCU |
| | GCUGCGGGGCGCCGUGAGCCAGAGACUGUAUAUACUUCUUCCUCUGGACUGCGGCGUGCCUGACAACCU |
| | GAGCAUGGCCGACCCUAACAUCAGAUUCCUGGACAAGCUGCCUCAGCAGACCGGCGACCACGCCGGCAUCA |
| | AGGACAGAGUGUACAGCAACUCCAUUUAUGAGCUGCUCGAGAAUGGCCAGAGAGCCGGCACCUGCGUGC |
| | UGGAGUACGCCACCCCUCUGCAGACCCUGUUCGCCAUGAGCCAGUACAGUCAGGCUGGAUUCAGCAGAGA |
| | GGACAGACUGGAGCAGGCCAAGCUGUUCUGCAGGACACUGGAGGACAUACUAGCAGACGCCCCUGAGAGC |
| | CAGAACAACUGCAGACUGAUUGCCUACCAGGAGCCUGCGGACGACAGCUCCUUCAGUCUGAGUCAGGAGG |
| | UGUUGCGGCACUUACGCCAAGAAGAGAAGGAGGAGGUGACCGUGGGCAGCCUGAAGACUAGCGCUGUGC |
| | CUAGCACCAGCACAAUGUCACAGGAGCCGGAAUUGCUAAUCAGCGGCAUGGAGAAGCCUCUCCCAUUACG |
| | UACCGACUUCAGC |
| | (KRAS(G12D)25 mer_ct.STING(V155M)) |
| 1464 | AUGCCUCACAGCAGCCUUCACCCUAGCAUCCCUUGCCCUAGAGGCCACGGCGCCCAGAAGGCCGCCCUAGU |
| | GCUCCUUAGCGCCUGCCUCGUGACCCUAUGGGGCUUAGGCGAGCCUCCAGAGCACACCUUGAGAUACCUC |
| | GUCCUCCACCUGGCUAGUCUACAGCUGGGCCUUCUCCUCAACGGCGUGUGCAGCCUGGCCGAGGAGCUG |
| | AGACACAUCCACAGCAGAUACAGAGGCAGCUACUGGAGAACCGUGAGAGCGUGCCUGGGCUGCCCUCUGA |
| | GAAGAGGCGCACUGCUGUUACUCAGCAUCUACUUCUACUACUCACUGCCAAACGCCGUGGGCCCUCCUUU |
| | CACCUGGAUGCUGGCCUUGCUCGGAUUGAGCCAGGCCCUGAACAUUUUACUGGGAUUGAAGGGCCUGGC |
| | CCCUGCCGAGAUCAGCGCCGUGUGCGAGAAGGGCAACUUCAACAUGGCCCACGGCCUAGCUUGGAGCUAC |
| | UACAUCGGCUACCUGAGACUGAUCCUGCCUGAGCUGCAGGCCAGAAUCAGAACCUACAACCAGCACUACA |
| | ACAACCUGCUGCGUGGAGCGGUGAGCCAGAGACUGUAUAUCCUCCUGCCUCUGGACUGCGGAGUGCCUG |
| | ACAACCUGAGCAUGGCCGACCCUAACAUCAGAUUCCUGGACAAGCUGCCUCAGCAGACCGGCGACCACGCC |
| | GGCAUCAAGGACAGAGUGUACAGCAACUCAAUCUACGAGCUGUUGGAGAAUGGCCAGAGAGCCGGCACC |
| | UGCGUGCUGGAGUACGCCACCCCUCUGCAGACCCUGUUCGCCAUGAGCCAGUACUCUCAGGCAGGCUUCA |
| | GCAGAGAGGACAGACUGGAGCAGGCCAAGCUGUUCUGCAGAACCCUGGAGGACAUCCUGGCGGACGCCCC |
| | UGAGAGCCAGAACAACUGCCGGCUUAUCGCCUACCAGGAGCCAGCAGACGACAGCAGCUUCUCUCUCUCA |
| | CAAGAGGUACUGCGCCAUCUUCGCCAGGAGGAGAAGGAGGAGGUGACCGUGGGCAGCCUGAAGACAUCC |
| | GCCGUACCUAGCACCAGCACCAUGUCUCAGGAACCGGAACUGUUGAUCAGCGGCAUGGAGAAGCCUCUGC |
| | CACUGCGCACCGACUUCAGCGCCACCAACUUCUCCCUACUGAAGCAAGCCGGUGACGUUGAAGAGAACCCU |
| | GGCCCUAUGACCGAGUACAAGCUGGUAGUAGUAGGCGCCGACGGCGUGGGCAAGAGCGCCCUGACCAUCC |
| | AGCUGAUCCAGAUGACUGAAUAUAAGCUUGUCGUCGUGGGCGCAGAUGGCGUUGGUAAGAGCGCACUU |
| | ACAAUUCAACUCAUUCAGAUGACGGAGUAUAAGCUGGUGGUGGUCGGAGCUGACGGCGUAGGCAAGAG |
| | UGCCCUUACUAUUCAGCUAAUUCAG |
| | (KRAS(G12D)25 mer^{∧}3_nt.STING(V155M)) |
| 1465 | AUGACCGAGUACAAGCUUGUGGUGGUUGGCGCCGACGGCGUGGGCAAGAGCGCCUUAACCAUCCAGCUU |
| | AUCCAGAUGACAGAGUAUAAGCUAGUGGUGGUCGGCGCAGACGGAGUGGGAAAGAGUGCAUUAACUAU |
| | UCAACUCAUCCAAAUGACCGAAUACAAGCUAGUAGUUGUGGGUGCAGAUGGCGUCGGCAAGUCUGCACU |
| | GACAAUUCAGCUCAUCCAGGCCACCAACUUCAGCCUGCUGAAGCAGGCCGGCGACGUGGAGGAGAACCCU |
| | GGCCCUAUGCCUCACAGCAGCCUGCACCCUAGCAUCCCUUGCCCUAGAGGCCACGGCGCCCAGAAGGCCGC |
| | CCUGGUGCUGCUGAGCGCCUGCCUGGUGACCCUGUGGGGCCUGGGCGAGCCUCCUGAGCACACCCUGAG |
| | AUACCUAGUUUUGCACCUGGCUUCUCUGCAGCUGGGCCUACUGCUCAACGGCGUGUGCAGCCUGGCCGA |
| | GGAGCUGAGACACAUCCACAGCAGAUACAGAGGCAGCUACUGGAGAACCGUGAGAGCAUGCUUAGGCUG |
| | CCCUCUGAGAAGAGGCGCUCUGCUCCUCUUGUCCAUCUACUUCUACUACUCGCUACCUAACGCCGUGGGC |
| | CCUCCUUUCACCUGGAUGCUGGCCCUCUUGGGAUUAAGCCAGGCCCUGAACAUCUUGCUGGGACUGAAG |
| | GGCCUGGCCCCUGCCGAGAUCAGCGCCGUGUGCGAGAAGGGCAACUUCAACAUGGCCCACGGACUCGCUU |
| | GGAGCUACUACAUCGGCUACCUGAGACUGAUCCUGCCUGAGCUGCAGGCCAGAAUCAGAACCUACAACCA |
| | GCACUACAACAACCUGCUGCGGGGAGCAGUGAGCCAGAGACUGUAUAUUCUGCUCCCUCUGGACUGCGG |
| | CGUGCCUGACAACCUGAGCAUGGCCGACCCUAACAUCAGAUUCCUGGACAAGCUGCCUCAGCAGACCGGC |
| | GACCACGCCGGCAUCAAGGACAGAGUGUACAGCAACAGCAUUUACGAGCUGCUGGAGAACGGCCAGAGAG |
| | CCGGCACCUGCGUGCUGGAGUACGCCACCCCUCUGCAGACCCUGUUCGCCAUGAGCCAGUACUCCCAGGC |
| | AGGAUUCAGCAGAGAGGACAGACUGGAGCAGGCCAAGCUGUUCUGCCGUACUCUUGAGGACAUCCUUGC |
| | AGACGCCCCUGAGAGCCAGAACAACUGCCGGUUGAUUGCCUACCAGGAACCGGCAGACGACAGCUCAUUC |
| | UCCUUGUCUCAGGAGGUCCUUAGACACCUGCGGCAGGAGGAGAAGGAGGAGGUGACCGUGGGCAGCCUG |
| | AAGACAUCCGCCGUGCCUAGCACGUCUACCAUGUCCCAGGAGCCGGAACUGCUAAUCAGCGGCAUGGAGA |
| | AGCCUCUGCCUCUCAGGACCGACUUCAGC |
| | (KRAS(G12D)25 mer^{∧}3_ct.STING(V155M)) |
| 1466 | AUGCCCCAUAGCAGCCUGCACCCCAGCAUCCCCUGCCCCAGAGGCCACGGCGCCCAGAAGGCCGCCCUGGU |
| | CCUGCUGAGCGCAUGCCUGGUCACCCUGUGGGGCCUGGGCGAGCCCCCCGAGCACACCCUGAGAUACCUG |
| | GUGCUGCACCUCGCCAGCCUGCAGCUGGGCCUGCUGCUGAACGGCGUGUGCAGCCUGGCCGAGGAGCUG |
| | AGACACAUCCACAGCAGAUAUAGAGGCAGCUACUGGAGAACCGUGAGAGCUUGCCUCGGCUGCCCCCUGA |
| | GAAGAGGCGCCCUGCUGCUGCUGAGCAUCUACUUUUACUACAGCCUGCCCAACGCUGUGGGCCCCCCUUU |
| | CACGUGGAUGCUCGCCCUGCUGGGACUGAGCCAGGCCCUGAACAUCCUGCUGGGCCUUAAGGGCCUAGCC |
| | CCCGCCGAGAUCAGCGCCGUGUGCGAGAAGGGCAACUUCAAUGUGGCCCACGGCCUGGCCUGGAGCUACU |
| | ACAUCGGCUACCUGAGACUGAUCCUGCCCGAGCUGCAGGCCAGAAUCAGAACCUACAAUCAGCACUACAAC |
| | AACCUGCUGAGAGGCGCCGUGAGCCAGAGACUGUACAUCCUGCUGCCCCUGGACUGCGGCGUGCCCGACA |
| | ACCUCAGCAUGGCCGACCCCAACAUCAGAUUCCUGGACAAGCUGCCCCAGCAGACCGGCGACCACGCCGGC |
| | AUCAAGGAUCGCGUGUACAGCAACAGCAUCUACGAGCUGCUGGAAAACGGCCAGAGAGCCGGAACCUGCG |
| | UGCUGGAGUACGCCACACCCCUGCAGACCCUGUUCGCCAUGAGCCAGUACAGCCAGGCCGGCUUCAGCAG |
| | AGAGGACAAGCUGGAGCAGGCCAAGCUGUUCUGCAGAACCCUGGAGGAUAUCCUCGCCGACGCCCCCGAG |
| | AGCCAGAACAACUGCAGGCUGAUCGCGUACCAGGAGCCCGCUGACGACAGCAGCUUUAGCCUGAGCCAGG |
| | AGGUGCUGAGACAUCUGCGUCAAGAGGAAAAGGAGGAGGUGACCGUGGGCUCCCUGAAGACCAGCGCCG |
| | UGCCCAGCACCAGCACCAUGAGCCAGGAGCCCGAGCUGCUGAUCAGCGGCAUGGAGAAGCCACUGCCCCUC |
| | AGAACCGACUUCAGCACC |
| | (Hu STING (R284K) var; no epitope tag) |
| 1467 | AUGAGAAUGAAGCAGCUGGAGGACAAGAUCGAGGAGCUGCUGAGCAAGAUCUACCACCUGGAGAACGAG |
| | AUCGCCAGACUGAAGAAGCUGAUCGGCGAGGCCGACCAGACCAGCGGCAACUACCUGAACAUGCAGGACA |
| | GCCAGGGCGUGCUGAGCAGCUUCCCCGCCCCCCAGGCCGUGCAGGACAACCCCGCCAUGCCCACCAGCAGC |
| | GGCAGCGAGGGCAACGUGAAGCUGUGCAGCCUGGAGGAGGCCCAGAGAAUCUGGAAGCAGAAGAGCGCC |
| | GAGAUCUACCCCAUCAUGGACAAGAGCAGCAGAACCAGACUGGCCCUGAUCAUCUGCAACGAGGAGUUCG |
| | ACAGCAUCCCCAGAAGAACCGGCGCCGAGGUGGACAUCACCGGCAUGACCAUGCUGCUGCAGAACCUGGG |
| | CUACAGCGUGGACGUGAAGAAGAACCUGACCGCCAGCGACAUGACCACCGAGCUGGAGGCCUUCGCCCAC |
| | AGACCCGAGCACAAGACCAGCGACAGCACCUUCCUGGUGUUCAUGAGCCACGGCAUCAGAGAGGGCAUCU |
| | GCGGCAAGAAGCACAGCGAGCAGGUGCCCGACAUCCUGCAGCUGAACGCCAUCUUCAACAUGCUGAACAC |
| | CAAGAACUGCCCCAGCCUGAAGGACAAGCCCAAGGUGAUCAUCAUCCAGGCCUGCAGAGGCGACAGCCCCG |
| | GCGUGGUGUGGUUCAAGGACAGCGUGGGCGUGAGCGGCAACCUGAGCCUGCCCACCACCGAGGAGUUCG |
| | AGGACGACGCCAUCAAGAAGGCCCACAUCGAGAAGGACUUCAUCGCCUUCUGCAGCAGCACCCCCGACAAC |
| | GUGAGCUGGAGACACCCCACCAUGGGCAGCGUGUUCAUCGGCAGACUGAUCGAGCACAUGCAGGAGUAC |
| | GCCUGCAGCUGCGACGUGGAGGAGAUCUUCAGAAAGGUGAGAUUCAGCUUCGAGCAGCCCGACGGCAGA |
| | GCCCAGAUGCCCACCACCGAGAGAGUGACCCUGACCAGAUGCUUCUACCUGUUCCCCGGCCAC |
| | DM_hsCASP1 (self-activating human Caspase 1); P2025 without epitope tag) |
| 1468 | AUGAGAAUGAAGCAGCUGGAGGACAAGAUCGAGGAGCUGCUGAGCAAGAUCUAUCACCUGGAGAACGAG |
| | AUCGCCAGACUGAAGAAGCUGAUCGGCGAGAGACAGAUCAGCCCCAACAAGAAGGCCCACCCCAACAUGGA |
| | GGCCGGACCGCCUGAGAGCGGCGAGAGCACCGACGCCCUGAAGCUGUGCCCCCACGAGGAGUUCCUGAGA |
| | CUGUGCAAGGAGAGAGCCGAGGAGAUCUACCCCAUCAAGGAGAGAAACAACAGAACCAGACUGGCCCUGA |
| | UCAUCUGCAACACCGAGUUCGACCACCUGCCCCCCAGAAACGGCGCCGACUUCGACAUCACCGGCAUGAAG |
| | GAGCUGCUGGAGGGCCUGGACUACAGCGUGGACGUGGAGGAGAACCUGACCGCCAGAGACAUGGAGAGC |
| | GCCCUGAGAGCCUUCGCCACCAGACCCGAGCACAAGAGCAGCGACAGCACCUUCCUGGUGCUGAUGAGCC |
| | ACGGCAUCCUGGAGGGCAUCUGCGGCACCGUGCACGACGAGAAGAAGCCCGACGUGCUGCUGUACGACAC |
| | CAUCUUCCAGAUCUUCAACAACAGAAACUGCCUGAGCCUGAAGGACAAGCCCAAGGUGAUCAUCGUGCAG |
| | GCCUGCAGAGGCGCCAACAGAGGCGAGCUGUGGGUGAGAGACAGCCCCGCCAGCCUGGAGGUGGCCAGCA |
| | GCCAGAGCAGCGAGAACCUGGAGGAGGACGCCGUGUACAAGACCCACGUGGAGAAGGACUUCAUCGCCUU |
| | CUGCAGCAGCACCCCCCACAACGUGAGCUGGAGAGACAGCACCAUGGGCAGCAUCUUCAUCACCCAGCUGA |
| | UAACCUGCUUCCAGAAGUACAGCUGGUGCUGCCACCUGGAGGAGGUGUUCAGAAAGGUGCAGCAGAGCU |
| | UCGAGACCCCCAGAGCCAAGGCCCAGAUGCCCACCAUCGAGAGACUGAGCAUGACCAGAUACUUCUACCUG |
| | UUCCCCGGCAAC |
| | (Caspase-4, N.del + DM domain; P2015 without epitope tag) |
| 1469 | AUGAGCGCCGAGGUGAUCCACCAGGUGGAGGAGGCCCUGGACACCGACGAGAAGGAGAUGCUGCUGUUC |
| | CUGUGCAGAGACGUGGCCAUCGACGUGGUGCCCCCCAACGUGAGAGACCUGCUGGACAUCCUGAGAGAG |
| | AGAGGCAAGCUGAGCGUGGGCGACCUGGCCGAGCUGCUGUACAGAGUGAGAAGAUUCGACCUGCUGAAG |
| | AGAAUCCUGAAGAUGGACAGAAAGGCCGUGGAGACCCACCUGCUGAGAAACCCCCACCUGGUGAGCGACU |
| | ACAGAGUGCUGAUGGCCGAGAUCGGCGAGGACCUGGACAAGAGCGACGUGAGCAGCCUGAUCUUCCUGA |
| | UGAAGGACUACAUGGGCAGAGGCAAGAUCAGCAAGGAGAAGAGCUUCCUGGACCUGGUGGUGGAGCUG |
| | GAGAAGCUGAACCUGGUGGCCCCCGACCAGCUGGACCUGCUGGAGAAGUGCCUGAAGAACAUCCACAGAA |
| | UCGACCUGAAGACCAAGAUCCAGAAGUACAAGCAGAGCGUGCAGGGCGCCGGCACCAGCUACAGAAACGU |
| | GCUGCAGGCCGCCAUCCAGAAGAGCCUGAAGGACCCCAGCAACAACUUCAGACUGCACAACGGCAGAAGCA |
| | AGGAGCAGAGACUGAAGGAGCAGCUGGGCGCCCAGCAGGAGCCCGUGAAGAAGAGCAUCCAGGAGAGCG |
| | AGGCCUUCCUGCCCCAGAGCAUCCCCGAGGAGAGAUACAAGAUGAAGAGCAAGCCCCUGGGCAUCUGCCU |
| | GAUCAUCGACUGCAUCGGCAACGAGACCGAGCUGCUGAGAGACACCUUCACCAGCCUGGGCUACGAGGUG |
| | CAGAAGUUCCUGCACCUGAGCAUGCACGGCAUCAGCCAGAUCCUGGGCCAGUUCGCCUGCAUGCCCGAGC |
| | ACAGAGACUACGACAGCUUCGUGUGCGUGCUGGUGAGCAGAGGCGGCAGCCAGAGCGUGUACGGCGUGG |
| | ACCAGACCCACAGCGGCCUGCCCCUGCACCACAUCAGAAGAAUGUUCAUGGGCGACAGCUGCCCCUACCUG |
| | GCCGGCAAGCCCAAGAUGUUCUUCAUCCAGAACUACGUGGUGAGCGAGGGCCAGCUGGAGGACAGCAGCC |
| | UGCUGGAGGUGGACGGCCCCGCCAUGAAGAACGUGGAGUUCAAGGCCCAGAAGAGAGGCCUGUGCACCG |
| | UGCACAGAGAGGCCGACUUCUUCUGGAGCCUGUGCACCGCCGACAUGAGCCUGCUGGAGCAGAGCCACAG |
| | CAGCCCCAGCCUGUACCUGCAGUGCCUGAGCCAGAAGCUGAGACAGGAGAGAAAGAGACCCCUGCUGGAC |
| | CUGCACAUCGAGCUGAACGGCUACAUGUACGACUGGAACAGCAGAGUGAGCGCCAAGGAGAAGUACUAC |
| | GUGUGGCUGCAGCACACCCUGAGAAAGAAGCUGAUCCUGAGCUACACC |
| | (hu-cFLIP-L; P1006 without epitope tag) |
| 1470 | AUGAGCGCCGAGGUGAUCCACCAGGUGGAGGAGGCCCUGGACACCGACGAGAAGGAGAUGCUGCUGUUC |
| | CUGUGCAGAGACGUGGCCAUCGACGUGGUGCCCCCCAACGUGAGAGACCUGCUGGACAUCCUGAGAGAG |
| | AGAGGCAAGCUGAGCGUGGGCGACCUGGCCGAGCUGCUGUACAGAGUGAGAAGAUUCGACCUGCUGAAG |
| | AGAAUCCUGAAGAUGGACAGAAAGGCCGUGGAGACCCACCUGCUGAGAAACCCCCACCUGGUGAGCGACU |
| | ACAGAGUGCUGAUGGCCGAGAUCGGCGAGGACCUGGACAAGAGCGACGUGAGCAGCCUGAUCUUCCUGA |
| | UGAAGGACUACAUGGGCAGAGGCAAGAUCAGCAAGGAGAAGAGCUUCCUGGACCUGGUGGUGGAGCUG |
| | GAGAAGCUGAACCUGGUGGCCCCCGACCAGCUGGACCUGCUGGAGAAGUGCCUGAAGAACAUCCACAGAA |
| | UCGACCUGAAGACCAAGAUCCAGAAGUACAAGCAGAGCGUGCAGGGCGCCGGCACCAGCUACAGAAACGU |
| | GCUGCAGGCCGCCAUCCAGAAGAGCCUGAAGGACCCCAGCAACAACUUCAGACUGCACAACGGCAGAAGCA |
| | AGGAGCAGAGACUGAAGGAGCAGCUGGGCGCCCAGCAGGAGCCCGUGAAGAAGAGC |
| | (hu-cFLIP-S(1-227); P1007 without epitope tag) |
| 1471 | AUGAGCGCCGAGGUGAUCCACCAGGUGGAGGAGGCCCUGGACACCGACGAGAAGGAGAUGCUGCUGUUC |
| | CUGUGCAGAGACGUGGCCAUCGACGUGGUGCCCCCCAACGUGAGAGACCUGCUGGACAUCCUGAGAGAG |
| | AGAGGCAAGCUGAGCGUGGGCGACCUGGCCGAGCUGCUGUACAGAGUGAGAAGAUUCGACCUGCUGAAG |
| | AGAAUCCUGAAGAUGGACAGAAAGGCCGUGGAGACCCACCUGCUGAGAAACCCCCACCUGGUGAGCGACU |
| | ACAGAGUGCUGAUGGCCGAGAUCGGCGAGGACCUGGACAAGAGCGACGUGAGCAGCCUGAUCUUCCUGA |
| | UGAAGGACUACAUGGGCAGAGGCAAGAUCAGCAAGGAGAAGAGCUUCCUGGACCUGGUGGUGGAGCUG |
| | GAGAAGCUGAACCUGGUGGCCCCCGACCAGCUGGACCUGCUGGAGAAGUGCCUGAAGAACAUCCACAGAA |
| | UCGACCUGAAGACCAAGAUCCAGAAGUACAAGCAGAGCGUGCAGGGCGCCGGCACCAGCUACAGAAACGU |
| | GCUGCAGGCCGCCAUCCAGAAGAGCCUGAAGGAC |
| | (hu-cFLIP-p22(1-198); P1008 without epitope tag)-nucleotide |
| 1472 | AUGAGCGCCGAGGUGAUCCACCAGGUGGAGGAGGCCCUGGACACCGACGAGAAGGAGAUGCUGCUGUUC |
| | CUGUGCAGAGACGUGGCCAUCGACGUGGUGCCCCCCAACGUGAGAGACCUGCUGGACAUCCUGAGAGAG |
| | AGAGGCAAGCUGAGCGUGGGCGACCUGGCCGAGCUGCUGUACAGAGUGAGAAGAUUCGACCUGCUGAAG |
| | AGAAUCCUGAAGAUGGACAGAAAGGCCGUGGAGACCCACCUGCUGAGAAACCCCCACCUGGUGAGCGACU |
| | ACAGAGUGCUGAUGGCCGAGAUCGGCGAGGACCUGGACAAGAGCGACGUGAGCAGCCUGAUCUUCCUGA |
| | UGAAGGACUACAUGGGCAGAGGCAAGAUCAGCAAGGAGAAGAGCUUCCUGGACCUGGUGGUGGAGCUG |
| | GAGAAGCUGAACCUGGUGGCCCCCGACCAGCUGGACCUGCUGGAGAAGUGCCUGAAGAACAUCCACAGAA |
| | UCGACCUGAAGACCAAGAUCCAGAAGUACAAGCAGAGCGUGCAGGGCGCCGGCACCAGCUACAGAAACGU |
| | GCUGCAGGCCGCCAUCCAGAAGAGCCUGAAGGACCCCAGCAACAACUUCAGACUGCACAACGGCAGAAGCA |
| | AGGAGCAGAGACUGAAGGAGCAGCUGGGCGCCCAGCAGGAGCCCGUGAAGAAGAGCAUCCAGGAGAGCG |
| | AGGCCUUCCUGCCCCAGAGCAUCCCCGAGGAGAGAUACAAGAUGAAGAGCAAGCCCCUGGGCAUCUGCCU |
| | GAUCAUCGACUGCAUCGGCAACGAGACCGAGCUGCUGAGAGACACCUUCACCAGCCUGGGCUACGAGGUG |
| | CAGAAGUUCCUGCACCUGAGCAUGCACGGCAUCAGCCAGAUCCUGGGCCAGUUCGCCUGCAUGCCCGAGC |
| | ACAGAGACUACGACAGCUUCGUGUGCGUGCUGGUGAGCAGAGGCGGCAGCCAGAGCGUGUACGGCGUGG |
| | ACCAGACCCACAGCGGCCUGCCCCUGCACCACAUCAGAAGAAUGUUCAUGGGCGACAGCUGCCCCUACCUG |
| | GCCGGCAAGCCCAAGAUGUUCUUCAUCCAGAACUACGUGGUGAGCGAGGGCCAGCUGGAGGACAGCAGCC |
| | UGCUGGAGGUGGAC |
| | (hu-cFLIP-p43(1-376); P1009 without epitope tag)-nucleotide |
| 1473 | AUGGGCCCCGCCAUGAAGAACGUGGAGUUCAAGGCCCAGAAGAGAGGCCUGUGCACCGUGCACAGAGAG |
| | GCCGACUUCUUCUGGAGCCUGUGCACCGCCGACAUGAGCCUGCUGGAGCAGAGCCACAGCAGCCCCAGCC |
| | UGUACCUGCAGUGCCUGAGCCAGAAGCUGAGACAGGAGAGAAAGAGACCCCUGCUGGACCUGCACAUCGA |
| | GCUGAACGGCUACAUGUACGACUGGAACAGCAGAGUGAGCGCCAAGGAGAAGUACUACGUGUGGCUGCA |
| | GCACACCCUGAGAAAGAAGCUGAUCCUGAGCUACACC |
| | (hu-cFLIP-p12(377-480); P1010 without epitope tag)-nucleotide |
| 1474 | AUGCAGCCCGACAUGAGCCUGAACGUGAUCAAGAUGAAGAGCAGCGACUUCCUGGAAUCGGCCGAGCUG |
| | GACAGCGGCGGCUUCGGCAAGGUGAGCCUGUGCUUCCACAGAACUCAGGGCCUGAUGAUCAUGAAGACC |
| | GUGUACAAGGGCCCCAAUUGCAUCGAGCACAACGAGGCCUUACUGGAGGAGGCCAAGAUGAUGAACAGA |
| | CUGAGACAUUCGAGAGUGGUCAAGUUACUGGGCGUGAUCAUCGAGGAAGGCAAGUACAGCCUGGUGAU |
| | GGAGUACAUGGAAAAGGGCAACCUGAUGCACGUGCUGAAGGCCGAGAUGAGCACCCCCCUGAGCGUGAA |
| | GGGCAGAAUCAUCCUGGAGAUUAUCGAGGGGAUGUGCUACCUGCACGGCAAGGGCGUGAUCCACAAGGA |
| | CCUGAAGCCGGAGAACAUCCUGGUGGACAACGACUUCCACAUCAAGAUCGCCGACCUGGGCCUGGCCAGC |
| | UUUAAGAUGUGGAGCAAGCUGAACAACGAGGAGCACAACGAGUUAAGAGAGGUGGACGGCACCGCCAAG |
| | AAGAACGGCGGCACCUUAUACUACAUGGCCCCCGAGCACCUGAACGAUGUGAACGCCAAGCCCACCGAGAA |
| | GAGCGACGUGUACUCCUUUGCCGUGGUCCUGUGGGCCAUCUUCGCCAACAAGGAGCCCUACGAGAACGCC |
| | AUUUGCGAGCAGCAGCUGAUCAUGUGCAUUAAGAGCGGCAACAGACCCGACGUGGACGACAUCACCGAG |
| | UACUGCCCCAGAGAGAUUAUCAGCCUGAUGAAGCUGUGCUGGGAGGCCAACCCCGAGGCUAGACCCACCU |
| | UCCCUGGGAUCGAGGAGAAAUUCAGACCCUUCUACCUGAGCCAGCUGGAGGAGAGCGUGGAAGAGGACG |
| | UGAAGAGCCUGAAGAAAGAGUACAGCAACGAGAACGCUGUGGUGAAGCGCAUGCAGAGCCUGCAGCUGG |
| | ACUGCGUGGCCGUCCCCAGCAGCAGAAGCAACAGUGCCACCGAGCAGCCGGGCUCGCUGCACUCCAGCCAG |
| | GGCCUGGGCAUGGGCCCCGUGGAGGAGAGCUGGUUCGCCCCCUCGCUGGAGCACCCCCAGGAGGAGAACG |
| | AACCUAGCCUGCAGAGCAAGCUGCAGGACGAGGCCAACUACCACCUGUACGGCAGCAGAAUGGACAGACA |
| | GACCAAGCAGCAACCAAGACAGAACGUGGCCUACAACAGAGAGGAGGAACGAAGAAGAAGAGUGAGCCAC |
| | GACCCCUUCGCCCAGCAGAGACCCUACGAGAACUUCCAGAACACCGAGGGCAAGGGCACCGCCUAUAGCAG |
| | CGCCGCCAGCCACGGCAACGCAGUGCACCAGCCCAGCGGCCUGACCUCUCAGCCCCAGGUGCUGUACCAGA |
| | AUAAUGGCCUGUAUAGCAGCCACGGCUUCGGCACCAGACCCCUGGACCCAGGCACCGCCGGCCCUAGAGU |
| | GUGGUACAGACCCAUCCCAAGCCACAUGCCCAGCCUGCACAACAUACCGGUGCCCGAGACAAACUACUUGG |
| | GCAACACCCCCACCAUGCCCUUCAGCAGCCUGCCCCCCACAGACGAGAGCAUCAAGUACACCAUCUAUAACA |
| | GCACCGGCAUCCAGAUCGGCGCCUACAACUAUAUGGAGAUCGGCGGUACCAGCAGCAGCGGCGGCAUCAA |
| | GAAGGAGAUAGAGGCAAUCAAGAAGGAGCAGGAGGCCAUCAAGAAGAAGAUCGAAGCCAUCGAGAAGGA |
| | GAUUGAGGCC |
| | (huRIPK1(1-555).IZ.TM; TH1021 without epitope tag)-nucleotide |
| 1475 | AUGCAGCCCGACAUGAGCCUGAAUGUGAUCAAGAUGAAGAGCAGCGACUUCCUGGAGAGCGCCGAGCUG |
| | GAUAGCGGCGGAUUCGGCAAGGUGAGCCUGUGCUUCCACAGAACCCAAGGCCUGAUGAUCAUGAAGACC |
| | GUGUACAAGGGACCCAACUGCAUCGAGCACAACGAAGCCCUGUUAGAGGAAGCCAAGAUGAUGAAUAGAC |
| | UGCGUCACUCUAGGGUGGUUAAACUGCUGGGCGUGAUCAUCGAGGAGGGCAAGUACAGCCUGGUGAUG |
| | GAGUACAUGGAGAAGGGCAACCUUAUGCACGUGCUGAAGGCCGAGAUGUCCACCCCCCUGAGCGUGAAG |
| | GGCAGAAUCAUCCUGGAGAUCAUCGAGGGAAUGUGUUAUCUGCAUGGCAAGGGCGUGAUCCACAAAGAC |
| | CUGAAGCCCGAGAACAUCCUGGUGGACAACGAUUUCCACAUCAAGAUCGCCGACCUGGGCCUGGCCAGCU |
| | UCAAGAUGUGGAGCAAGCUGAACAACGAGGAGCACAACGAACUGAGAGAGGUGGAUGGCACCGCCAAGA |
| | AAAACGGCGGCACCCUGUAUUACAUGGCCCCCGAGCACCUGAACGACGUGAACGCCAAGCCCACCGAGAAG |
| | AGCGACGUUUACAGCUUUGCCGUGGUGCUGUGGGCCAUCUUCGCCAACAAGGAGCCCUACGAGAACGCC |
| | AUCUGCGAGCAGCAGCUGAUCAUGUGCAUCAAGAGCGGCAACAGACCCGACGUGGACGACAUCACCGAGU |
| | ACUGCCCCCGUGAGAUCAUUAGCCUGAUGAAGCUGUGCUGGGAGGCCAACCCCGAGGCCAGACCCACCUU |
| | CCCCGGCAUUGAGGAGAAGUUCAGACCCUUCUACCUGAGCCAGUUAGAGGAAAGCGUGGAGGAGGACGU |
| | GAAAAGCCUGAAGAAAGAGUACUCUAACGAGAACGCCGUGGUGAAACGCAUGCAGAGCCUGCAGCUGGA |
| | UUGCGUGGCCGUGCCCAGCUCCAGAAGCAACAGCGCCACCGAACAACCUGGCAGCCUGCACAGCUCCCAGG |
| | GCCUGGGCAUGGGCCCCGUGGAGGAGAGCUGGUUCGCCCCCUCCCUGGAGCAUCCGCAGGAGGAGAACG |
| | AGCCCUCUCUGCAGUCCAAGCUGCAAGACGAGGCCAACUACCACCUGUACGGCAGCAGAAUGGACAGACA |
| | GACCAAGCAGCAACCCAGACAAAAUGUGGCCUACAAUAGAGAGGAGGAGAGAAGAAGAAGAGUGAGCCAC |
| | GACCCUUUCGCCCAGCAGAGACCCUACGAGAACUUCCAGAAUACCGAGGGCAAGGGUACCGCCUACAGCU |
| | CAGCGGCCUCGCACGGCAACGCCGUGCACCAGCCCAGCGGCCUGACCAGCCAGCCCCAGGUGCUGUACCAA |
| | AACAACGGCCUGUAUAGCUCCCACGGCUUUGGCACCAGACCCCUGGACCCCGGCACCGCCGGCCCCAGAGU |
| | CUGGUAUAGACCCAUCCCCAGCCAUAUGCCUAGCCUGCACAACAUCCCCGUGCCCGAGACCAACUACCUGG |
| | GCAAUACCCCCACCAUGCCGUUCAGCAGCUUACCCCCCACCGACGAGAGCAUCAAGUACACCAUCUACAAC |
| | AGCACCGGCAUCCAGAUCGGCGCCUACAACUACAUGGAAAUCGGCGGAACCAGCAGCAGCGGCAGCGACG |
| | GCAGCGGCUCCGGAAGCGGAAGCAUAACCAUCAGGGCCGCCUUCCUGGAGAAGGAAAAUACCGCGCUGAG |
| | AACAGAGAUUGCCGAGUUAGAAAAGGAGGUGGGCAGAUGCGAGAACAUAGUGAGCAAGUACGAGACCAG |
| | AUACGGCCCCCUG |
| | (huRIPK1(1-555).EE.DM; TH1022 without epitope tag)-nucleotide |
| 1476 | AUGCAACCCGACAUGAGCUUGAACGUGAUCAAGAUGAAGAGCAGCGAUUUCCUGGAGAGCGCCGAGCUG |
| | GACAGCGGCGGCUUCGGCAAGGUGAGCCUGUGUUUCCACAGAACCCAGGGCCUGAUGAUCAUGAAGACA |
| | GUGUACAAGGGCCCCAACUGCAUCGAGCACAACGAGGCCCUGCUGGAGGAGGCUAAGAUGAUGAACAGAC |
| | UGAGACACAGCAGAGUCGUGAAGCUGCUGGGCGUGAUCAUCGAAGAGGGCAAGUACAGCCUGGUGAUG |
| | GAGUACAUGGAGAAAGGCAACCUUAUGCACGUGCUCAAGGCCGAGAUGAGCACCCCUCUGAGCGUGAAG |
| | GGAAGAAUCAUCCUGGAGAUCAUCGAGGGCAUGUGCUACCUGCACGGCAAGGGCGUCAUCCAUAAGGAC |
| | CUGAAGCCCGAGAAUAUCCUUGUGGACAACGACUUCCAUAUCAAGAUCGCCGACCUCGGCCUGGCCAGCU |
| | UCAAGAUGUGGAGCAAGCUGAACAACGAGGAGCACAACGAGCUGAGAGAGGUAGACGGCACCGCCAAGAA |
| | AAAUGGCGGCACCCUGUACUACAUGGCUCCCGAGCACCUGAAUGACGUGAACGCCAAGCCUACCGAAAAG |
| | AGCGACGUGUAUAGCUUCGCCGUGGUGCUCUGGGCCAUCUUCGCCAACAAGGAGCCUUAUGAGAAUGCA |
| | AUCUGCGAGCAGCAGCUGAUCAUGUGCAUCAAGAGCGGCAACAGACCCGACGUGGACGACAUCACCGAAU |
| | ACUGCCCCAGAGAGAUCAUCAGCCUGAUGAAGCUGUGCUGGGAGGCCAACCCCGAGGCCAGACCCACCUU |
| | CCCCGGCAUUGAGGAGAAGUUCAGACCCUUCUACCUGAGCCAGUUGGAAGAGAGCGUGGAGGAGGACGU |
| | CAAAAGCCUGAAGAAGGAGUACAGCAACGAGAACGCCGUCGUGAAGAGAAUGCAGAGCCUGCAGCUGGAC |
| | UGCGUGGCCGUGCCUAGCAGCAGAAGCAACAGCGCCACCGAGCAGCCCGGCAGCCUGCACAGCAGCCAGG |
| | GCCUUGGAAUGGGCCCCGUGGAGGAAAGCUGGUUCGCCCCCAGCCUUGAGCAUCCGCAGGAGGAGAACG |
| | AGCCCAGCCUGCAGAGCAAGCUGCAGGACGAAGCCAACUAUCACCUGUACGGCAGCAGAAUGGACCGACA |
| | GACCAAGCAGCAGCCCAGACAGAACGUGGCCUAUAACCGAGAGGAGGAGAGAAGAAGAAGGGUGAGCCAC |
| | GACCCCUUCGCCCAACAGAGACCCUACGAGAACUUCCAGAACACCGAGGGCAAGGGCACCGCUUACAGUAG |
| | CGCCGCAAGCCACGGCAACGCCGUGCACCAACCUAGCGGACUGACCAGCCAGCCCCAGGUGCUGUACCAAA |
| | ACAACGGUCUGUACAGCUCACACGGCUUCGGGACCAGACCCUUAGAUCCCGGAACCGCCGGCCCCAGAGU |
| | AUGGUAUAGACCCAUCCCCAGCCACAUGCCCAGCUUGCACAACAUCCCCGUGCCCGAGACCAACUACCUGG |
| | GCAACACCCCCACCAUGCCCUUCAGCAGCCUGCCCCCCACCGACGAGAGCAUCAAAUAUACCAUCUACAACA |
| | GCACCGGAAUCCAGAUCGGGGCCUACAAUUACAUGGAGAUCGGAGGCACCAGCAGCAGCGGCAGCGACGG |
| | UAGCGGAAGCGGCAGCGGCAGCCUCGAGAUCAGAGCCGCCUUCCUGGAGAAGGAGAACACCGCCCUGAGA |
| | ACCAGAGCCGCCGAACUGAGAAAGAGAGUGGGCAGAUGCAGAAACAUCGUGAGCAAGUACGAGACCAGAU |
| | ACGGCCCCCUG |
| | (huRIPK1(1-555).RR.DM; TH1023 without epitope tag)-nucleotide |
| 1477 | AUGCAGCCUGACAUGAGCCUGGACAAUAUCAAGAUGGCCAGCAGCGACCUGCUCGAGAAGACCGACCUGG |
| | ACAGUGGCGGCUUCGGAAAAGUGAGCCUGUGCUACCACAGGUCUCACGGGUUCGUGAUCCUGAAGAAGG |
| | UGUACACCGGCCCCAACAGAGCCGAGUAUAAUGAGGUGCUGCUGGAGGAGGGCAAGAUGAUGCACAGAC |
| | UGAGACAUAGCAGAGUGGUGAAGCUGCUGGGCAUCAUCAUCGAGGAGGGAAACUACAGCCUGGUUAUG |
| | GAGUACAUGGAGAAGGGCAACCUAAUGCACGUGUUGAAGACCCAGAUAGACGUGCCACUGAGCUUAAAG |
| | GGCAGAAUCAUCGUGGAGGCUAUCGAGGGCAUGUGCUACCUGCACGACAAGGGCGUGAUCCACAAAGAC |
| | CUGAAGCCCGAGAACAUACUCGUGGAUAGAGAUUUCCACAUCAAGAUCGCCGACCUGGGCGUGGCCAGCU |
| | UCAAGACUUGGAGCAAGCUGACAAAGGAGAAGGACAACAAGCAGAAGGAGGUGAGCAGCACCACCAAGAA |
| | AAACAACGGCGGCACCCUGUACUACAUGGCCCCUGAGCACCUGAACGACAUCAACGCCAAGCCCACCGAGA |
| | AGAGCGACGUGUAUAGCUUCGGCAUCGUGCUGUGGGCCAUCUUUGCUAAGAAAGAGCCCUACGAGAACG |
| | UGAUCUGCACCGAGCAGUUCGUCAUCUGCAUCAAGAGCGGCAACAGACCCAAUGUGGAGGAGAUCCUGG |
| | AAUACUGCCCCAGAGAGAUCAUCAGCCUCAUGGAGAGAUGCUGGCAGGCCAUCCCUGAGGACAGACCCAC |
| | CUUCCUGGGCAUUGAGGAGGAGUUCAGACCCUUCUACCUGAGCCACUUCGAGGAGUACGUGGAGGAGGA |
| | CGUGGCCAGUCUGAAAAAGGAGUAUCCAGACCAGAGCCCCGUGCUGCAGAGAAUGUUCAGCCUGCAGCAC |
| | GACUGUGUGCCCCUGCCCCCCAGCAGAAGCAACAGCGAGCAGCCGGGCAGCCUGCACAGCAGCCAGGGCU |
| | UACAAAUGGGACCCGUGGAGGAGAGCUGGUUCAGCAGUAGCCCCGAGUACCCCCAGGACGAGAACGACAG |
| | GUCGGUCCAGGCCAAGCUCCAGGAAGAGGCCAGCUACCACGCCUUCGGCAUCUUCGCCGAGAAGCAAACC |
| | AAGCCCCAGCCCAGACAAAACGAAGCCUACAACAGAGAGGAAGAGAGAAAGAGACGCGUAAGCCACGACCC |
| | CUUUGCCCAACAGAGAGCCAGAGAAAACAUCAAGAGCGCCGGCGCCCGGGGCCACUCGGAUCCGAGCACCA |
| | CUAGCAGAGGCAUCGCUGUGCAGCAACUCAGCUGGCCCGCCACCCAGACCGUGUGGAACAACGGCCUGUA |
| | CAACCAGCACGGCUUCGGCACCACCGGCACCGGCGUUUGGUACCCCCCCAACCUGUCGCAGAUGUACAGCA |
| | CCUACAAAACCCCCGUGCCCGAGACCAACAUCCCCGGCAGCACCCCCACCAUGCCCUAUUUCAGCGGCCCCG |
| | UGGCCGACGACCUGAUCAAGUACACCAUCUUCAACAGCAGCGGCAUCCAGAUCGGCAACCACAAUUACAU |
| | GGACGUGGGCCUGAACAGCCAGCCACCCAACAACACCUGCAAGGAAGAAAGCACCAGCGGCGGCAUCAAGA |
| | AGGAAAUCGAGGCCAUCAAGAAGGAGCAGGAAGCCAUAAAGAAGAAAAUCGAGGCCAUCGAGAAGGAGA |
| | UCGAGGCC |
| | (msRIPK1(1-555).IZ.TM; TH1024 without epitope tag)-nucleotide |
| 1478 | AUGCAGCCCGACAUGAGCCUGGACAACAUUAAGAUGGCCAGUAGCGACCUGCUGGAGAAGACCGACCUGG |
| | AUAGCGGGGGCUUCGGCAAGGUGAGCCUGUGCUACCACAGAAGCCACGGAUUCGUGAUCCUGAAGAAGG |
| | UGUACACCGGCCCCAACAGAGCCGAGUACAACGAGGUGCUGCUGGAGGAGGGCAAGAUGAUGCAUAGAC |
| | UGAGACACAGCAGAGUGGUGAAACUGCUGGGGAUCAUCAUCGAAGAGGGCAACUAUAGCCUGGUGAUG |
| | GAAUACAUGGAGAAGGGCAACCUGAUGCACGUGCUGAAGACCCAGAUCGACGUGCCCCUGAGCCUGAAG |
| | GGCAGAAUCAUCGUGGAGGCCAUCGAGGGUAUGUGCUACCUGCACGAUAAGGGCGUGAUCCACAAGGAC |
| | CUGAAACCUGAAAACAUCUUAGUGGACAGAGACUUCCACAUCAAGAUCGCCGACCUGGGAGUGGCUAGC |
| | UUCAAGACCUGGAGCAAACUGACCAAGGAGAAGGAUAACAAGCAGAAGGAAGUGAGCAGCACCACCAAGA |
| | AAAACAACGGAGGCACCCUGUACUACAUGGCCCCCGAGCAUCUGAACGACAUCAACGCCAAGCCCACCGAG |
| | AAGAGCGACGUGUACUCCUUCGGCAUCGUCUUAUGGGCCAUCUUCGCCAAGAAGGAGCCCUACGAGAAC |
| | GUGAUCUGCACCGAACAGUUUGUGAUCUGCAUCAAGAGCGGCAAUAGACCCAACGUGGAGGAGAUCCUG |
| | GAGUACUGCCCCAGAGAGAUCAUCAGCCUGAUGGAGAGGUGCUGGCAGGCUAUCCCCGAGGACAGACCCA |
| | CCUUUCUGGGCAUCGAGGAAGAGUUCAGACCCUUCUAUCUGAGCCACUUCGAGGAGUAUGUUGAGGAG |
| | GACGUGGCCAGCCUGAAGAAGGAGUACCCCGACCAGAGCCCCGUGCUGCAGAGAAUGUUCAGCCUGCAAC |
| | ACGAUUGCGUGCCGCUGCCCCCCAGCAGAUCGAAUAGCGAGCAGCCAGGCAGCCUACACAGCAGUCAGGG |
| | CCUGCAGAUGGGCCCCGUGGAGGAAAGCUGGUUCAGCAGCAGCCCCGAGUACCCCCAGGACGAGAAUGAC |
| | AGAAGCGUGCAAGCAAAGCUGCAAGAGGAGGCCAGCUACCACGCCUUCGGCAUCUUCGCCGAGAAACAGA |
| | CUAAGCCCCAGCCCAGACAGAACGAGGCCUACAACAGAGAGGAGGAGAGAAAAAGACGAGUGAGCCACGA |
| | CCCCUUCGCCCAGCAGAGAGCCAGAGAGAAUAUCAAGAGCGCCGGCGCCAGAGGCCACAGCGACCCCAGCA |
| | CCACCAGCAGAGGAAUCGCCGUGCAGCAGCUGAGCUGGCCCGCCACCCAGACCGUGUGGAACAACGGCCU |
| | GUACAACCAGCACGGCUUUGGCACCACCGGCACCGGCGUGUGGUAUCCCCCCAACCUGAGCCAGAUGUAC |
| | AGCACCUAUAAAACCCCUGUGCCGGAGACCAAUAUCCCCGGCAGCACCCCUACCAUGCCCUACUUCAGCGG |
| | CCCCGUGGCCGACGACCUGAUCAAGUACACGAUCUUCAACAGCAGCGGCAUCCAGAUAGGCAACCACAACU |
| | ACAUGGACGUGGGCCUGAACAGCCAACCCCCCAAUAACACCUGCAAGGAGGAGUCCACCAGCGGCAGCGAC |
| | GGCAGCGGCAGCGGCAGCGGCAGCAUAACCAUCAGAGCUGCUUUCCUGGAGAAGGAGAACACCGCUCUGA |
| | GAACCGAGAUCGCCGAGCUGGAGAAGGAGGUCGGCAGAUGCGAGAAUAUCGUGAGCAAGUACGAGACCA |
| | GAUACGGACCCCUG |
| | (msRIPK1(1-555).EE.DM; TH1025 without epitope tag)-nucleotide |
| 1479 | AUGCAGCCUGAUAUGAGCCUGGACAACAUCAAGAUGGCCAGCAGCGACUUGCUGGAGAAGACCGAUCUG |
| | GACUCCGGCGGCUUUGGCAAGGUGAGCCUGUGUUACCACAGAAGCCACGGCUUCGUGAUCCUGAAAAAG |
| | GUGUACACCGGCCCCAAUAGAGCAGAGUACAACGAGGUGCUGCUGGAGGAGGGCAAGAUGAUGCACAGA |
| | CUGAGGCAUAGCAGAGUGGUGAAACUGCUGGGCAUCAUCAUUGAGGAGGGCAACUACAGCCUGGUGAU |
| | GGAGUACAUGGAGAAGGGCAACCUGAUGCAUGUGCUGAAGACCCAAAUCGACGUGCCCCUGUCGCUGAA |
| | GGGCAGAAUCAUCGUGGAGGCCAUCGAGGGGAUGUGCUACCUGCACGACAAGGGCGUGAUCCACAAGGA |
| | CCUGAAGCCCGAGAACAUCCUGGUGGAUAGAGACUUCCACAUCAAGAUCGCCGACCUGGGCGUUGCCAGC |
| | UUCAAGACCUGGUCUAAACUGACCAAGGAGAAAGACAACAAGCAGAAGGAGGUGAGCAGCACCACCAAGA |
| | AGAACAACGGCGGAACACUGUACUAUAUGGCCCCUGAGCACCUGAACGACAUCAACGCCAAGCCCACCGAG |
| | AAAAGCGAUGUUUACAGCUUCGGCAUCGUGCUGUGGGCCAUCUUCGCCAAGAAGGAGCCCUACGAGAAC |
| | GUGAUCUGCACCGAGCAGUUCGUGAUCUGCAUCAAGAGCGGCAACAGACCCAACGUGGAGGAAAUCCUG |
| | GAGUACUGCCCCAGAGAGAUCAUCAGCCUGAUGGAGAGAUGCUGGCAGGCCAUCCCCGAGGACCGUCCCA |
| | CGUUCCUGGGCAUCGAAGAGGAGUUCCGGCCCUUCUACCUGAGCCAUUUCGAGGAGUAUGUGGAGGAG |
| | GACGUGGCCAGCCUGAAGAAGGAGUACCCCGACCAGAGCCCAGUGCUGCAGAGAAUGUUCAGCCUUCAAC |
| | ACGACUGCGUGCCCCUGCCUCCCUCAAGAAGCAACAGCGAGCAGCCCGGCAGCUUGCACAGCAGCCAGGGC |
| | CUGCAGAUGGGCCCCGUGGAGGAGAGCUGGUUUAGCAGCAGCCCCGAGUACCCCCAGGACGAGAAUGACA |
| | GAAGCGUGCAAGCCAAGUUACAGGAGGAGGCCAGCUACCACGCCUUUGGAAUCUUCGCCGAGAAGCAGAC |
| | CAAGCCCCAGCCCAGACAGAACGAGGCCUACAACAGAGAGGAGGAGAGAAAAAGAAGAGUGAGCCACGACC |
| | CCUUCGCCCAGCAGAGAGCCAGAGAGAACAUUAAGAGCGCCGGCGCGAGAGGCCACAGCGACCCCAGCACC |
| | ACAAGCAGAGGCAUCGCCGUGCAGCAAUUGAGCUGGCCCGCCACCCAGACCGUGUGGAACAACGGCCUGU |
| | AUAACCAGCACGGCUUCGGAACCACCGGCACCGGCGUGUGGUACCCCCCCAAUCUGAGCCAGAUGUACAG |
| | CACUUACAAGACCCCCGUGCCCGAAACCAACAUCCCCGGCAGCACCCCCACCAUGCCCUACUUCAGCGGCCC |
| | CGUGGCCGACGACCUCAUCAAGUACACAAUAUUUAACAGCAGCGGCAUCCAGAUCGGCAACCACAACUACA |
| | UGGACGUGGGCCUGAACAGCCAGCCCCCGAACAAUACCUGCAAGGAGGAGAGCACAAGCGGCUCUGACGG |
| | CAGCGGCAGCGGCAGCGGCUCACUGGAGAUCAGAGCUGCCUUCCUGGAAAAGGAGAACACCGCUCUGAGA |
| | ACCAGAGCCGCCGAGCUGCGAAAGAGAGUAGGCAGAUGCAGAAACAUCGUGAGCAAGUACGAGACCAGAU |
| | ACGGUCCCCUG |
| | (msRIPK1(1-555).RR.DM; TH1026 without epitope tag)-nucleotide |
| 1480 | AUGAGCGCCGGCGACCCCAGAGUGGGCAGCGGCAGCCUGGACAGCUUCAUGUUCAGCAUCCCCCUGGUGG |
| | CCCUGAACGUGGGCGUGAGAAGAAGACUGAGCCUGUUCCUGAACCCCAGAACCCCCGUGGCCGCCGACUG |
| | GACCCUGCUGGCCGAGGAGAUGGGCUUCGAGUACCUGGAGAUCAGAGAGCUGGAGACCAGACCCGACCCC |
| | ACCAGAAGCCUGCUGGACGCCUGGCAGGGCAGAAGCGGCGCCAGCGUGGGCAGACUGCUGGAGCUGCUG |
| | GCCCUGCUGGACAGAGAGGACAUCCUGAAGGAGCUGAAGAGCAGAAUCGAGGAGGACUGCCAGAAGUAC |
| | CUGGGCAAGCAGCAGAACCAGGAGAGCGAGAAGCCCCUGCAGGUGGCCAGAGUGGAGAGCAGCGUGCCCC |
| | AGACCAAGGAGCUGGGCGGCAUCACCACCCUGGACGACCCCCUGGGCCAGACCCCCGAGCUGUUCGACGCC |
| | UUCAUCUGCUACUGCCCCAACGACAUCGAGUUCGUGCAGGAGAUGAUCAGACAGCUGGAGCAGACCGACU |
| | ACAGACUGAAGCUGUGCGUGAGCGACAGAGACGUGCUGCCCGGCACCUGCGUGUGGAGCAUCGCCAGCG |
| | AGCUGAUCGAGAAGAGAUGCAGAAGAAUGGUGGUGGUGGUGAGCGACGACUACCUGCAGAGCAAGGAG |
| | UGCGACUUCCAGACCAAGUUCGCCCUGAGCCUGAGCCCCGGCGUGCAGCAGAAGAGACCCAUCCCCAUCAA |
| | GUACAAGGCCAUGAAGAAGGACUUCCCCAGCAUCCUGAGAUUCAUCACCAUCUGCGACUACACCAACCCCU |
| | GCACCAAGAGCUGGUUCUGGACCAGACUGGCCAAGGCCCUGAGCCUGCCC |
| | (human myd88(L265P); P4027 without epitope tag)-nucleotide |
| 1481 | AUGGGCGUGGGCAAGAGCAAGCUGGACAAGUGCCCCCUGAGCUGGCACAAGAAGGACAGCGUGGACGCC |
| | GACCAGGACGGCCACGAGAGCGACAGCAAGAACAGCGAGGAGGCCUGCCUGAGAGGCUUCGUGGAGCAGA |
| | GCAGCGGCAGCGAGCCCCCCACCGGCGAGCAGGACCAGCCCGAGGCCAAGGGCGCCGGCCCCGAGGAGCAG |
| | GACGAGGAGGAGUUCCUGAAGUUCGUGAUCCUGCACGCCGAGGACGACACCGACGAGGCCCUGAGAGUG |
| | CAGGACCUGCUGCAGAACGACUUCGGCAUCAGACCCGGCAUCGUGUUCGCCGAGAUGCCCUGCGGCAGAC |
| | UGCACCUGCAGAACCUGGACGACGCCGUGAACGGCAGCGCCUGGACCAUCCUGCUGCUGACCGAGAACUU |
| | CCUGAGAGACACCUGGUGCAACUUCCAGUUCUACACCAGCCUGAUGAACAGCGUGAGCAGACAGCACAAG |
| | UACAACAGCGUGAUCCCCAUGAGACCCCUGAACAGCCCCCUGCCCAGAGAGAGAACCCCCCUGGCCCUGCA |
| | GACCAUCAACGCCCUGGAGGAGGAGAGCCAGGGCUUCAGCACCCAGGUGGAGAGAAUCUUCAGAGAGAGC |
| | GUGUUCGAGAGACAGCAGAGCAUCUGGAAGGAGACCAGAAGCGUGAGCCAGAAGCAGUUCAUCGCC |
| | (Mouse TRAM (TICAM2); P4033 without epitope tag)-nucleotide |
| 1482 | AUGAGCACCGCCAGCGCCGCCAGCUCAAGCUCCUCCUCUAGCGCCGGCGAGAUGAUCGAGGCCCCCAGCCA |
| | GGUGCUGAACUUCGAGGAGAUCGACUACAAGGAAAUCGAGGUGGAGGAGGUGGUGGGCAGAGGCGCCU |
| | UCGGCGUGGUGUGCAAGGCCAAGUGGAGAGCCAAGGACGUGGCCAUCAAGCAGAUCGAGAGCGAGUCCG |
| | AGAGAAAGGCCUUCAUCGUGGAGCUGAGACAGCUGAGCAGAGUGAACCACCCCAACAUCGUGAAGCUGU |
| | ACGGCGCCUGCCUGAACCCCGUGUGCCUGGUGAUGGAGUACGCCGAGGGCGGCAGCCUGUACAACGUGC |
| | UGCACGGCGCCGAGCCCCUGCCCUACUACACCGCCGCCCACGCCAUGAGCUGGUGCCUGCAGUGCAGCCAG |
| | GGCGUGGCCUACCUGCACAGCAUGCAGCCCAAGGCCCUGAUCCACCGCGAUCUGAAGCCCCCCAACCUGCU |
| | GCUGGUGGCCGGCGGCACCGUGCUGAAGAUCUGCGACUUCGGCACCGCCUGCGACAUCCAGACCCACAUG |
| | ACCAACAACAAGGGAUCAGCUGCGUGGAUGGCCCCCGAGGUGUUCGAGGGCAGCAACUACAGCGAGAAG |
| | UGCGACGUGUUCAGCUGGGGCAUCAUCCUGUGGGAGGUGAUCACCAGAAGAAAGCCCUUCGACGAGAUC |
| | GGCGGCCCCGCCUUCAGAAUCAUGUGGGCCGUGCACAACGGCACCAGACCGCCGCUGAUCAAGAACCUGC |
| | CCAAGCCCAUCGAGUCCCUGAUGACCAGAUGCUGGAGCAAGGACCCGAGCCAGAGGCCCAGCAUGGAAGA |
| | GAUCGUUAAGAUCAUGACCCACCUGAUGAGAUACUUCCCGGGCGCCGAUGAACCGCUGCAGUACCCCUGC |
| | CAGGAGUUCGGCGGAGGCGGCGGCCAGAGCCCCACCCUGACCCUGCAGAGCACCAACACCCACACCCAGAG |
| | CAGCAGCAGUAGCAGCGACGGCGGCCUGUUCAGAAGCAGACCCGCCCACAGCCUGCCCCCCGGCGAGGACG |
| | GCAGAGUGGAGCCCUACGUGGACUUCGCCGAGUUCUACAGACUGUGGAGCGUGGACCACGGCGAGCAGA |
| | GCGUGGUGACCGCCCCC |
| | (human TAK1-TAB1; P4031 without epitope tag)-nucleotide |
| 1483 | AUGGAGAACCUGAAGCACAUCAUCACCCUGGGCCAGGUGAUCCACAAGAGAUGCGAGGAGAUGAAGUAC |
| | UGCAAGAAGCAGUGCAGAAGACUGGGCCACAGAGUGCUGGGCCUGAUCAAGCCCCUGGAGAUGCUGCAG |
| | GACCAGGGCAAGAGAAGCGUGCCCAGCGAGAAGCUGACCACCGCCAUGAACAGAUUCAAGGCCGCCCUGG |
| | AGGAGGCCAACGGCGAGAUCGAGAAGUUCAGCAACAGAAGCAACAUCUGCAGAUUCCUGACCGCCAGCCA |
| | GGACAAGAUCCUGUUCAAGGACGUGAACAGAAAGCUGAGCGACGUGUGGAAGGAGCUGAGCCUGCUGCU |
| | GCAGGUGGAGCAGAGAAUGCCCGUGAGCCCCAUCAGCCAGGGCGCCAGCUGGGCCCAGGAGGACCAGCAG |
| | GACGCCGACGAGGACAGAAGAGCCUUCCAGAUGCUGAGAAGAGACAACGAGAAGAUCGAGGCCAGCCUGA |
| | GAAGACUGGAGAUCAACAUGAAGGAGAUCAAGGAGACCCUGAGACAGUAC |
| | (human MLKL(1-180) ORF nucleotide sequence; no epitope tag) |
| 1484 | AUGGAGCACGACCUUGAGAGAGGCCCUCCGGGCCCUAGAAGACCUCCUCGAGGUCCUCCACUUAGCAGCA |
| | GCUUGGGCCUCGCUCUCUUAUUGUUGCUACUUGCCUUGUUGUUCUGGUUGUACAUCGUGAUGAGCGAC |
| | UGGACCGGCGGCGCCCUUCUGGUGCUGUACAGCUUCGCCCUGAUGCUGAUCAUUAUCAUACUGAUUAUC |
| | UUCAUAUUCAGAAGAGAUCUGCUGUGCCCUCUGGGCGCCUUAUGCAUUCUGCUUUUGAUGAUCACUCU |
| | GCUCCUCAUCGCACUCUGGAACCUGCACGGCCAGGCCCUGUUCCUGGGCAUCGUGCUGUUCAUCUUCGGC |
| | UGCCUCCUCGUGCUUGGAAUCUGGAUCUACCUGCUGGAGAUGCUGUGGAGACUAGGUGCCACCAUCUGG |
| | CAGCUGCUGGCCUUCUUCCUGGCAUUCUUCUUAGACCUGAUUCUGCUCAUUAUUGCCCUAUACCUGCAG |
| | CAGAACUGGUGGACCCUACUCGUUGAUCUCCUGUGGCUACUGCUGUUCCUUGCUAUCCUGAUUUGGAU |
| | GUACUACCACGGACAAAGACCUUUCGCCGAGGACAAGACCUACAAGUACAUCUGCAGAAACUUCAGCAAC |
| | UUCUGCAACGUGGACGUGGUGGAGAUCCUGCCUUACCUGCCUUGCCUGACCGCCAGGGACCAGGACAGA |
| | CUGAGAGCCACCUGCACCCUGAGCGGCAACAGAGACACCCUGUGGCACCUGUUCAACACCCUGCAGAGGC |
| | GCCCUGGCUGGGUGGAGUACUUCAUCGCCGCCCUGAGAGGCUGCGAGUUGGUUGACCUCGCCGACGAGG |
| | UGGCCAGCGUGUACCAGAGCUACCAGCCUAGAACCAGCGACAGGCCGCCUGACCCUCUGGAGCCUCCUAG |
| | CCUGCCUGCCGAACGGCCUGGCCCACCUACCCCUGCCGCCGCCCACAGCAUCCCUUACAACUCCUGUCGGG |
| | AGAAGGAGCCUAGCUACCCUAUGCCUGUGCAGGAAACGCAGGCCCCAGAAAGUCCUGGCGAGAACAGCGA |
| | GCAGGCCUUGCAGACUCUGAGCCCUAGAGCCAUCCCUAGAAACCCUGACGGCGGUCCUCUCGAGAGUUCC |
| | AGCGACCUGGCUGCACUCUCCCCACUGACCAGCAGCGGCCACCAGGAGCAGGACACCGAGCUGGGCAGCAC |
| | CCACACCGCCGGCGCUACCUCAAGCCUUACCCCUAGCCGGGGCCCAGUCAGCCCUAGCGUGAGCUUCCAGC |
| | CUCUGGCCAGAAGCACACCAAGAGCCAGCAGACUUCCAGGACCAACCGGCAGCGUGGUGAGCACCGGCACC |
| | AGCUUCAGUUCCUCUAGCCCAGGCUUAGCCAGCGCCGGAGCGGCCGAGGGCAAGCAGGGCGCCGAGAGCG |
| | ACCAGGCCGAGCCUAUCAUCUGUUCCUCGGGUGCCGAGGCCCCUGCCAACAGCCUACCUAGCAAGGUGCC |
| | UACCACACUGAUGCCAGUUAACACCGUGGCCCUGAAGGUUCCAGCCAACCCUGCUUCCGUUUCUACAGUG |
| | CCGUCCAAGCUGCCGACGUCAUCCAAGCCUCCGGGAGCCGUGCCAUCUAACGCCCUGACCAAUCCAGCUCC |
| | AAGCAAGCUCCCAAUCAACAGCACCAGAGCCGGCAUGGUGCCUUCAAAGGUGCCGACCUCCAUGGUGCUG |
| | ACCAAGGUGAGCGCCUCUACCGUGCCAACCGACGGAUCUUCUCGGAACGAGGAGACACCUGCUGCUCCUA |
| | CUCCAGCGGGCGCAACUGGAGGCUCCUCGGCUUGGCUGGACAGUUCUAGCGAGAAUAGAGGCCUGGGUA |
| | GUGAGCUGAGUAAGCCGGGCGUGCUCGCAAGCCAGGUGGACAGCCCUUUCAGCGGCUGCUUCGAAGACC |
| | UUGCAAUUUCCGCAUCUACCAGUCUAGGCAUGGGCCCUUGCCACGGCCCUGAGGAGAACGAGUACAAGA |
| | GCGAGGGCACCUUCGGCAUCCACGUGGCCGAGAACCCUAGCAUCCAGCUGCUUGAGGGCAAUCCUGGACC |
| | ACCAGCCGAUCCUGAUGGCGGACCUAGACCUCAGGCCGACAGAAAGUUCCAGGAGAGAGAGGUGCCUUG |
| | UCAUAGACCUUCCCCAGGCGCUCUUUGGCUGCAGGUGGCCGUGACCGGUGUCCUCGUCGUGACAUUACU |
| | GGUGGUGCUCUACAGAAGAAGACUGCAC |
| | (CA-hMAVS ORF nucleotide sequence; no epitope tag) |
| 1485 | AUGAGCUGGUCCCCAAGCCUCACGACCCAGACCUGCGGCGCUUGGGAGAUGAAGGAGAGACUGGGCACG |
| | GGGGGCUUUGGCAACGUGAUCAGAUGGCAUAAUCAGGAAACCGGAGAGCAGAUUGCUAUCAAGCAGUG |
| | UAGACAGGAGCUAAGCCCCCGCAAUAGAGAGAGGUGGUGCCUGGAAAUUCAGAUUAUGAGAAGACUGAC |
| | CCAUCCCAAUGUGGUCGCCGCAAGAGACGUCCCCGAAGGCAUGCAGAACCUGGCCCCCAAUGACCUGCCUC |
| | UUCUGGCCAUGGAAUACUGCCAGGGCGGCGACCUGCGGAAGUACCUGAAUCAGUUUGAAAAUUGCUGCG |
| | GCCUGAGAGAGGGCGCCAUAUUGACACUGCUGAGCGACAUCGCCAGCGCCCUGAGAUACCUGCACGAGAA |
| | CAGAAUAAUUCACAGAGACCUGAAGCCGGAGAAUAUUGUGCUGCAGCAGGGUGAACAGAGGCUCAUCCA |
| | UAAGAUCAUCGACCUGGGGUACGCCAAGGAGCUGGAUCAGGGCGAGCUGUGUACCGAGUUUGUGGGGA |
| | CUCUGCAAUACCUGGCCCCCGAGCUCCUGGAACAGCAGAAGUACACCGUCACAGUGGAUUAUUGGAGCUU |
| | CGGCACGCUGGCCUUCGAGUGCAUCACGGGCUUUAGGCCGUUUCUGCCCAAUUGGCAGCCCGUGCAAUG |
| | GCACAGCAAGGUCAGACAGAAAAGCGAGGUCGACAUCGUAGUGAGCGAAGACCUGAACGGCACUGUCAAG |
| | UUCAGUAGCUCCCUCCCCUACCCUAACAAUCUGAACAGCGUGCUGGCAGAGCGGCUGGAGAAGUGGCUAC |
| | AACUAAUGCUGAUGUGGCACCCCCGACAGCGUGGCACCGACCCCACCUACGGGCCCAACGGAUGCUUCAA |
| | GGCCCUGGACGACAUUCUCAACCUGAAGCUGGUGCACAUCUUGAAUAUGGUGACCGGCACCAUCCACACC |
| | UACCCCGUGACCGAAGACGAAAGCUUGCAGAGCCUGAAGGCCAGAAUUCAACAGGACACAGGCAUCCCCG |
| | AAGAGGAUCAAGAGCUGCUGCAGGAAGCCGGCCUGGCUUUGAUUCCCGACAAACCAGCCACCCAGUGCAU |
| | UAGCGACGGCAAGCUGAACGAGGGCCACACCCUGGACAUGGACCUGGUGUUCCUGUUCGACAACAGCAAG |
| | AUUACCUACGAGACCCAAAUCAGCCCAAGGCCCCAACCCGAGAGCGUGAGCUGCAUCCUGCAAGAGCCCAA |
| | GAGGAAUCUGGCCUUCUUCCAACUAAGAAAGGUGUGGGGCCAAGUGUGGCACAGCAUCCAGACUCUGAA |
| | GGAAGACUGCAAUAGACUGCAACAAGGACAGCGAGCCGCCAUGAUGAACCUGUUAAGAAACAACAGCUGC |
| | UUAUCUAAGAUGAAGAACAGCAUGGCCUCCAUGAGCCAGCAGCUGAAAGCCAAACUGGAUUUCUUCAAG |
| | ACCAGCAUCCAGAUCGACCUGGAGAAGUACAGCGAGCAGACGGAGUUCGGGAUCACCAGCGACAAGCUGC |
| | UGCUGGCUUGGAGGGAAAUGGAACAGGCCGUGGAGCUGUGCGGCAGAGAGAACGAGGUUAAACUGCUG |
| | GUAGAGCGGAUGAUGGCCCUGCAGACCGACAUUGUAGACCUCCAGAGAAGCCCUAUGGGAAGAAAACAG |
| | GGCGGAACACUGGACGACCUGGAGGAGCAGGCUAGAGAGCUGUACAGAAGACUUAGAGAGAAGCCCAGA |
| | GACCAAAGAACCGAGGGCGACAGCCAGGAGAUGGUGAGACUGCUGCUACAGGCUAUUCAAAGUUUCGAG |
| | AAGAAAGUGAGAGUGAUCUACACCCAACUCAGCAAAACCGUGGUGUGUAAGCAGAAGGCCCUGGAGCUG |
| | CUGCCCAAGGUUGAGGAGGUUGUCAGCCUGAUGAAUGAGGAUGAGAAGACCGUGGUGAGACUGCAAGA |
| | GAAAAGGCAGAAAGAACUGUGGAACCUUUUAAAGAUUGCCUGCAGCAAGGUGAGGGGCCCUGUAUCAGG |
| | AUCCCCCGACUCUAUGAACGCCAGCAGACUGAGCCAGCCCGGUCAACUGAUGAGCCAGCCCUCUACCGCCA |
| | GCAACUCCCUGCCCGAGCCAGCCAAGAAGAGCGAGGAACUGGUGGCCGAGGCCCACAAUCUGUGCACCCU |
| | ACUGGAGAACGCCAUUCAGGACACCGUUCGCGAGCAGGACCAGAGCUUCACCGCCCUGGACUGGAGCUGG |
| | CUGCAGACUGAGGAGGAAGAGCACAGCUGCCUGGAGCAGGCCAGC |
| | (huIKK2ca(S177E/S181E); P4005 without epitope tag)-nucleotide |
| 1486 | AUGAGCAGCGUGAAGCUCUGGCCCACCGGCGCCAGCGCCGUGCCCCUAGUGAGCCGGGAGGAGCUUAAGA |
| | AGCUCGAGUUCGUGGGCAAGGGCGGCUUCGGCGUGGUGUUCCGGGCCCACCACCGGACCUGGAACCACG |
| | ACGUGGCCGUGAAGAUCGUGAACAGCAAGAAGAUCAGCUGGGAGGUGAAGGCCAUGGUGAACCUGCGGA |
| | ACGAGAACGUGUUGCUGCUGCUGGGCGUGACCGAGGACCUGCAGUGGGACUUCGUGAGCGGCCAGGCCU |
| | UGGUUACCCGGUUCAUGGAGAACGGCAGCCUGGCCGGCCUGCUGCAGCCCGAGUGCCCCCGGCCCUGGCC |
| | CCUGCUGUGCCGGCUACUGCAGGAGGUGGUGCUGGGCAUGUGCUACCUGCACAGCCUGAACCCCCCACU |
| | UCUGCACCGGGACCUGAAGCCCAGCAACAUCCUGCUGGACCCCGAGCUGCACGCCAAGCUGGCCGACUUCG |
| | GCCUGAGCACCUUCCAGGGCGGCAGCCAGAGCGGCUCCGGAUCUGGCAGCGGAAGCCGGGACAGCGGCGG |
| | CACCCUGGCCUACCUGGACCCAGAGCUGCUGUUCGACGUGAACCUCAAGGCCAGCAAGGCCUCCGACGUG |
| | UACAGCUUCGGCAUCCUGGUGUGGGCCGUGCUGGCUGGAAGGGAGGCCGAGCUGGUGGACAAGACCAGC |
| | CUGAUCCGGGAGACAGUGUGCGACCGGCAGAGCCGGCCUCCUCUCACCGAACUGCCCCCCGGCAGCCCCGA |
| | GACUCCUGGCCUGGAGAAGCUGAAGGAGCUCAUGAUCCACUGCUGGGGCUCCCAGAGCGAGAACCGGCCC |
| | AGCUUCCAGGACUGCGAGCCCAAGACCAACGAGGUGUACAACCUGGUGAAGGACAAGGUGGACGCCGCCG |
| | UGAGCGAGGUCAAGCACUACCUGAGCCAGCACCGGAGCAGCGGCCGGAACCUGAGCGCCCGGGAGCCCAG |
| | CCAGCGGGGCACCGAGAUGGACUGUCCUCGCGAGACAAUGGUGAGCAAGAUGCUGGAUCGGCUGCACCU |
| | GGAGGAGCCUUCAGGCCCCGUGCCCGGCAAGUGUCCUGAGAGACAGGCCCAGGACACCAGCGUGGGCCCU |
| | GCCACCCCUGCACGGACCAGCAGCGACCCCGUGGCCGGCACCCCCCAGAUCCCCCACACCCUGCCCUUCAGA |
| | GGCACCACUCCAGGCCCGGUGUUCACGGAGACACCUGGACCACACCCCCAGCGGAACCAGGGCGACGGUAG |
| | ACACGGCACACCAUGGUACCCAUGGACACCUCCUAACCCCAUGACCGGUCCACCUGCCCUGGUGUUCAACA |
| | ACUGCAGCGAGGUGCAGAUCGGCAACUACAACAGCCUGGUGGCCCCUCCUAGGACCACCGCCAGCAGCAG |
| | CGCCAAGUACGAUCAGGCACAGUUCGGCCGGGGCAGAGGUUGGCAGCCCUUCCACAAGGGAGGAAUCAA |
| | GAAGGAGAUCGAGGCCAUUAAGAAGGAACAGGAAGCUAUAAAGAAGAAGAUUGAAGCUAUCGAGAAGGA |
| | AAUUGAGGCC |
| | (muRIPK3-IZ.Tri mer; TH1015 with no epitope tag)-nucleotide |
| 1487 | AUGGCCGCUCUGAAGUCAUGGCUCUCAAGAAGUGUGACCAGCUUCUUCAGGUAUAGGCAGUGCCUGUGC |
| | GUGCCGGUCGUUGCUAACUUUAAAAAACGCUGUUUCAGCGAGCUGAUUCGCCCAUGGCACAAAACCGUG |
| | ACCAUCGGGUUCGGAGUCACACUGUGCGCUGUCCCAAUCGCACAAGCUGUGUAUACGCUUACCUCACUU |
| | UACAGACAGUACACAUCUUUGCUGGGAAAGAUGAAUUCUGAGGAGGAAGACGAGGUGUGGCAAGUUAU |
| | UAUUGGCGCCAGAGCCGAAAUGACAUCGAAGCAUCAGGAAUACCUGAAACUUGAGACCACAUGGAUGAC |
| | GGCAGUCGGACUCUCCGAGAUGGCAGCCGAAGCAGCCUACCAGACAGGUGCCGACCAGGCUAGCAUCACA |
| | GCUCGGAACCAUAUCCAAUUGGUAAAGCUGCAGGUCGAAGAGGUCCACCAACUAAGCCGAAAAGCCGAAA |
| | CCAAACUGGCUGAAGCCCAGAUUGAAGAACUGCGGCAAAAAACCCAGGAAGAGGGCGAGGAGCGAGCCGA |
| | AUCUGAGCAAGAAGCUUAUCUGCGGGAAGAU |
| | (Diablo.3; TH2003 without epitope tag)-nucleotide |
| 1488 | AUGGGCUGCGUGUGCAGCAGCAACCCCGAGGACGACUGGAUGGAGAACGGCGGCAUCAAGAAGGAGAUA |
| | GAAGCCAUUAAGAAAGAGCAGGAGGCCAUCAAGAAGAAGAUCGAGGCCAUCGAGAAGGAGAUCGAAGCC |
| | GGCAGCGGCGGCGGCAGCGGCAGUGGCGGCGGCAGCGACCCCUUCCUGGUGCUGCUGCACAGCUUAAGC |
| | GGCAGCCUGAGCGGCAACGACCUGAUGGAGCUGAAGUUCCUGUGUAGAGAGAGAGUGAGCAAGAGAAAG |
| | CUGGAGAGAGUGCAGAGCGGCCUGGACCUGUUCACCGUGCUGCUGGAGCAGAACGACCUGGAAAGAGGC |
| | CACACCGGCUUGCUGAGAGAGUUGCUGGCCUCACUGAGAAGACACGAUCUGCUGCAGAGACUGGACGAC |
| | UUCGAGGCCGGCACCGCCACCGCCGCCCCCCCCGGAGAAGCCGACCUGCAGGUGGCCUUCGACAUCGUGUG |
| | CGACAACGUGGGCAGAGACUGGAAGAGAUUGGCCAGAGAGCUGAAGGUGAGCGAGGCCAAGAUGGACGG |
| | CAUCGAGGAGAAGUACCCCAGAAGCCUGAGCGAGAGAGUGAGAGAGAGCCUGAAGGUGUGGAAGAACGC |
| | CGAGAAGAAGAACGCCAGCGUGGCUGGGCUGGUGAAGGCCCUGAGAACCUGCAGACUGAACCUGGUGGC |
| | CGAUCUGGUGGAGGAGGCCCAGGAGAGCGUGAGCAAGAGCGAGAACAUGAGCCCCGUGCUGAGAGACAG |
| | CACCGUGAGUAGCAGCGAGACCCCC |
| | (Myr(Lck)-IZ-L-msFADD; TH3002 without epitope tag)-nucleotide |
| 1489 | AUGGGCUGCGUGUGCAGCAGCAACCCCGAGGACGACUGGAUGGAGAACGGCGGCAUCAAAAAGGAGAUC |
| | GAGGCCAUCAAGAAGGAGCAGGAGGCCAUCAAGAAGAAGAUCGAGGCCAUCGAGAAAGAGAUAGAGGCC |
| | GGCAGCGGCGGCGGCAGCGGCAGCGGCGGCGGCAGCCCCGGCGAGGAGGACCUGUGCGCCGCCUUCAACG |
| | UGAUCUGCGACAACGUGGGCAAGGACUGGAGAAGACUGGCCAGACAGCUGAAGGUGAGCGACACCAAGA |
| | UCGACAGCAUCGAGGACAGAUACCCCAGAAACCUGACCGAGAGAGUGAGAGAGAGCCUGAGAAUCUGGAA |
| | GAACACCGAGAAGGAGAACGCCACCGUGGCCCACCUGGUGGGCGCCCUGAGAAGCUGCCAGAUGAACCUG |
| | GUGGCCGACCUGGUGCAGGAGGUGCAGCAGGCCAGAGACCUGCAGAACAGAAGCGGCGCCAUGAGCCCCA |
| | UGAGCUGGAACAGC |
| | (Myr(Lck)-IZ-L-huFADD-DD; TH3003 without epitope tag)-nucleotide |
| 1490 | AUGGGCUGCGUGUGCAGCAGCAACCCCGAGGACGACUGGAUGGAGAACGGCGGCAUCAAGAAAGAGAUC |
| | GAGGCCAUCAAAAAGGAGCAGGAGGCCAUCAAGAAGAAGAUCGAGGCCAUCGAGAAGGAGAUCGAGGCC |
| | GGCUCUGGCGGCGGCAGCGGCAGCGGCGGCGGCAGCCCCCCCGGCGAGGCCGACUUACAGGUGGCCUUCG |
| | ACAUCGUGUGCGACAACGUGGGCAGAGACUGGAAGAGACUGGCCAGAGAGCUGAAGGUGAGCGAGGCCA |
| | AGAUGGACGGCAUCGAGGAGAAGUACCCCAGAAGCCUGAGCGAGAGAGUGAGAGAGAGCCUGAAGGUGU |
| | GGAAGAACGCCGAGAAGAAGAACGCCAGCGUGGCCGGCCUGGUGAAGGCCCUGAGAACCUGCAGACUGAA |
| | CCUGGUGGCCGACCUGGUGGAGGAGGCCCAGGAGAGCGUGAGCAAGAGCGAGAACAUGAGCCCCGUGCU |
| | GAGAGACAGCACCGUGAGC |
| | (Myr(Lck)-IZ-L-msFADD-DD; TH3004 without epitope tag)-nucleotide |
| 1491 | AUGGGCCAGACCGUGACCACCCCCCUGAGCCUCACCCUGGAUCACUGGGGCGGCAUCAAGAAAGAGAUCG |
| | AGGCCAUCAAGAAGGAGCAGGAGGCCAUCAAGAAGAAGAUCGAAGCCAUCGAGAAGGAGAUCGAGGCCG |
| | GCAGCGGCGGCGGCAGCGGCAGCGGCGGCGGCAGCGACCCCUUCCUGGUGCUGCUGCACAGCGUGUCCAG |
| | CAGCCUGAGCAGCAGCGAGCUGACCGAGCUGAAGUUCCUGUGCCUGGGCAGAGUGGGCAAAAGAAAGCU |
| | GGAGAGAGUGCAGAGCGGCCUGGACCUCUUCAGCAUGCUGCUGGAGCAGAACGACUUGGAGCCCGGCCA |
| | CACCGAGCUGCUGAGAGAGCUGCUGGCCAGCCUGCGGAGACACGACCUGCUGAGAAGAGUGGAUGACUU |
| | CGAGGCCGGCGCCGCCGCCGGCGCCGCCCCCGGCGAGGAGGACCUGUGCGCCGCCUUCAACGUGAUCUGC |
| | GACAACGUGGGCAAGGAUUGGAGAAGAUUAGCCAGACAGCUGAAGGUGAGUGACACCAAGAUUGACAGC |
| | AUCGAGGACAGAUACCCCAGAAACCUGACCGAGAGAGUCAGAGAGAGCCUGAGAAUCUGGAAGAAUACCG |
| | AGAAGGAGAACGCCACCGUGGCCCACCUGGUGGGCGCCCUGAGAAGCUGCCAGAUGAACCUGGUGGCCGA |
| | CCUGGUGCAGGAGGUGCAGCAGGCCAGAGACCUGCAGAACAGAAGCGGCGCCAUGAGCCCCAUGAGCUGG |
| | AACAGCGACGCCAGCACCAGCGAGGCCAGC |
| | (Myr(MMSV)-IZ-L-huFADD; TH3005 without epitope tag)-nucleotide |
| 1492 | AUGGGCCAGACAGUGACCACCCCCCUGUCCCUGACCUUGGACCACUGGGGCGGCAUCAAGAAGGAGAUCG |
| | AGGCCAUCAAGAAGGAGCAGGAGGCCAUCAAAAAGAAGAUCGAAGCCAUUGAGAAGGAGAUCGAGGCCG |
| | GAAGCGGGGGCGGCAGCGGCAGCGGCGGAGGAAGCGACCCCUUCCUGGUGCUGCUGCAUAGCCUGUCAG |
| | GCAGCCUGAGCGGCAACGAUCUGAUGGAGCUGAAGUUCCUGUGCCGCGAGAGAGUGAGCAAGAGAAAGC |
| | UGGAGAGAGUACAGAGCGGCCUGGACCUGUUCACCGUGCUGCUGGAGCAGAAUGACCUGGAGAGAGGCC |
| | ACACCGGCUUGCUGAGAGAGUUGCUGGCCAGCCUGAGAAGGCACGACCUGCUGCAGAGACUGGACGACU |
| | UCGAGGCCGGCACCGCCACCGCCGCCCCCCCCGGCGAAGCGGACCUGCAGGUGGCCUUCGACAUCGUGUGC |
| | GACAACGUGGGCAGAGACUGGAAGAGACUGGCCAGAGAACUGAAGGUGAGCGAGGCCAAAAUGGACGGC |
| | AUCGAGGAGAAGUACCCCAGAAGCCUGAGCGAGAGAGUGAGAGAGAGCCUGAAGGUGUGGAAGAACGCC |
| | GAGAAGAAGAACGCCAGCGUGGCCGGCCUGGUGAAGGCCCUGAGAACAUGCAGACUGAACCUGGUGGCC |
| | GAUCUUGUGGAGGAGGCCCAGGAGAGCGUGAGCAAGAGCGAAAACAUGAGCCCCGUGCUGAGAGACAGC |
| | ACCGUGAGCAGCAGCGAGACCCCC |
| | (Myr(MMSV)-IZ-L-msFADD; TH3006 without epitope tag)-nucleotide |
| 1493 | AUGGGCCAGACCGUGACCACCCCCCUGAGCCUGACCCUGGACCACUGGGGCGGCAUCAAGAAGGAGAUCG |
| | AGGCCAUCAAGAAGGAGCAGGAGGCCAUCAAGAAGAAGAUUGAGGCUAUCGAGAAGGAGAUCGAGGCCG |
| | GCAGCGGCGGCGGCAGCGGCAGCGGCGGCGGCAGCCCCGGCGAGGAGGACCUGUGCGCCGCCUUCAACGU |
| | GAUCUGCGACAACGUGGGCAAGGACUGGAGAAGACUGGCCAGACAGCUGAAGGUGAGCGACACCAAGAU |
| | CGACAGCAUCGAGGACAGAUACCCCAGAAACCUGACCGAGAGAGUGAGAGAGAGCCUGAGAAUCUGGAAG |
| | AACACCGAGAAGGAGAACGCCACCGUGGCCCACCUGGUGGGCGCCCUGAGAAGCUGCCAGAUGAACCUGG |
| | UGGCCGACCUGGUGCAGGAGGUGCAGCAGGCCAGAGACCUGCAGAACAGAAGCGGCGCCAUGAGCCCCAU |
| | GAGCUGGAACAGC |
| | (Myr(MMSV)-IZ-L-huFADD-DD; TH3007 without epitope tag)-nucleotide |

1. A messenger RNA (mRNA) encoding a polypeptide that enhances an immune response to an antigen of interest in a subject, wherein the immune response comprises a cellular or humoral immune response characterized by:
(i) stimulating Type I interferon pathway signaling;
(ii) stimulating NFκB pathway signaling;
(iii) stimulating an inflammatory response;
(iv) stimulating cytokine production;
(v) stimulating dendritic cell development, activity or mobilization; or
(vi) a combination of any of (i)-(v).

2.-3. (canceled)

4. The mRNA of claim 1, wherein the polypeptide (i) stimulates a Type I IFN response, and wherein the polypeptide is selected from the group consisting of STING, MAVS, IRF1, IRF3, IRF5, IRF7, IRF8, IRF9, TBK1, IKKα, IKKi, MyD88, TRAM, TRAF3, TRAF6, IRAK1, IRAK4, TRIF, IPS-1, RIG-1, DAI and IFI16; (ii) stimulates an NFκB-mediated proinflammatory response and is selected from the group consisting of STING, c-FLIP, IKKβ, RIPK1, Btk, TAK1, TAK-TAB1, TBK1, MyD88, IRAK1, IRAK2, IRAK4, TAB2, TAB3, TRAF6, TRAM, MKK3, MKK4, MKK6 and MKK7U; (iii) is an intracellular adaptor protein selected from the group consisting of STING, MAVS and MyD88; (iv) is an intracellular signaling protein is selected from the group consisting of MyD88, IRAK 1, IRAK2, IRAK4, TRAF3, TRAF6, TAK1, TAB2, TAB3, TAK-TAB1, MKK3, MKK4, MKK6, MKK7, IKKα, IKKβ, TRAM, TRIF, RIPK1, and TBK1; (v) is a transcription factor, wherein the transcription factor is IRF3 or IRF7; (vi) is involved in necroptosis or necroptosome formation and selected from the group consisting of MLKL, RIPK1, RIPK3, DIABLO and FADD; or (vii) is involved in pyroptosis or inflammasome formation and selected from the group consisting of caspase 1, caspase 4, caspase 5, caspase 11, GSDMD, NLRP3, Pyrin domain and ASC/PYCARD.

5.-16. (canceled)

17. A lipid nanoparticle comprising the mRNA of claim 1.

18. (canceled)

19. A composition comprising a first mRNA encoding a first polypeptide that enhances an immune response to an antigen of interest in a subject, a second mRNA encoding a second polypeptide that enhances an immune response to an antigen of interest in a subject and, optionally, a third mRNA encoding a third polypeptide that enhances an immune response to an antigen of interest in a subject, wherein the first, second and third polypeptides function downstream of at least one Toll-like receptor (TLR) to thereby enhance an immune response, and wherein the immune response comprises a cellular or humoral immune response characterized by:
(i) stimulating Type I interferon pathway signaling;
(ii) stimulating NFκB pathway signaling;
(iii) stimulating an inflammatory response;
(iv) stimulating cytokine production;
(v) stimulating dendritic cell development, activity or mobilization; or
(vi) a combination of any of (i)-(v).

20. The composition of claim 19, wherein:
(i) the first polypeptide stimulates a Type I interferon (IFN) response and the second polypeptide stimulates an NFκB-mediated proinflammatory response;
(ii) the first polypeptide stimulates a Type I interferon (IFN) response and the second polypeptide is involved in necroptosis or necroptosome formation;
(iii) the first polypeptide stimulates a Type I interferon (IFN) response and the second polypeptide is involved in pyroptosis or inflammasome formation;
(iv) the first polypeptide stimulates an NFκB-mediated proinflammatory response and the second polypeptide is involved in necroptosis or necroptosome formation;
(v) the first polypeptide stimulates an NFκB-mediated proinflammatory response and the second polypeptide is involved in pyroptosis or inflammasome formation;
(vi) the first polypeptide stimulates a Type I interferon (IFN) response, the second polypeptide stimulates an NFκB-mediated proinflammatory response and the third polypeptide is involved in necroptosis or necroptosome formation; or
(vii) the first polypeptide stimulates a Type I interferon (IFN) response, the second polypeptide stimulates an NFκB-mediated proinflammatory response and the third polypeptide is involved in pyroptosis or inflammasome formation.

21. The composition of claim 20, wherein (i) the first polypeptide stimulates a Type I interferon (IFN) response and is selected from the group consisting of STING, MAVS, IRF1, IRF3, IRF5, IRF7, IRF8, IRF9, TBK1, IKKα, IKKi, MyD88, TRAM, TRAF3, TRAF6, IRAK1, IRAK4, TRIF, IPS-1, RIG-1, DAI and IFI16; and the second polypeptide stimulates an NFκB-mediated proinflammatory response and is selected from the group consisting of STING, c-FLIP, IKKβ, RIPK1, Btk, TAK1, TAK-TAB1, TBK1, MyD88, IRAK1, IRAK2, IRAK4, TAB2, TAB3, TRAF6, TRAM, MKK3, MKK4, MKK6 and MKK7;
(ii) the first polypeptide is a constitutively active IRF3 and the second polypeptide is a constitutively active IKKα;
(iii) the first polypeptide stimulates a Type I interferon (IFN) response and is selected from the group consisting of STING, MAVS, IRF1, IRF3, IRF5, IRF7, IRF8, IRF9, TBK1, IKKα, IKKi, MyD88, TRAM, TRAF3, TRAF6, IRAK1, IRAK4, TRIF, IPS-1, RIG-1, DAI and IFI16; and the second polypeptide is involved in necroptosis or necroptosome formation and is selected from the group consisting of MLKL, RIPK1, RIPK3, DIABLO and FADD;
(iv) the first polypeptide is a constitutively active STING and the second polypeptide is an MLKL polypeptide;
(v) the first polypeptide stimulates an NFκB-mediated proinflammatory response and is selected from the group consisting of STING, c-FLIP, IKKβ, RIPK1, Btk, TAK1, TAK-TAB1, TBK1, MyD88, IRAK1, IRAK2, IRAK4, TAB2, TAB3, TRAF6, TRAM, MKK3, MKK4, MKK6 and MKK7; and the second polypeptide is involved in pyroptosis or inflammasome formation and is selected from the group consisting of caspase 1, caspase 4, caspase 5, caspase 11, GSDMD, NLRP3, Pyrin domain and ASC/PYCARD; or
(vi) the first polypeptide is a constitutively active IKKβ and the second polypeptide is a caspase-1 polypeptide.

22.-29. (canceled)

30. A lipid nanoparticle comprising the composition of claim 19.

31.-33. (canceled)

34. A kit comprising a container comprising the lipid nanoparticle of claim 30, and an optional pharmaceutically acceptable carrier, and a package insert comprising instructions for administration of the lipid nanoparticle or composition for enhancing an immune response in an individual.

35.-43. (canceled)

44. A method of enhancing an immune response to an antigen of interest in a subject, the method comprising administering the lipid nanoparticle of claim 30 to the subject such that an immune response to the antigen of interest is enhanced.

45. A composition comprising at least one immune potentiator mRNA, and at least one mRNA encoding an antigen of interest, wherein the immune potentiator functions downstream of at least one Toll-like receptor (TLR) to thereby enhance an immune response, and wherein the immune response comprises a cellular or humoral immune response characterized by:
(i) stimulating Type I interferon pathway signaling;
(ii) stimulating NFκB pathway signaling;
(iii) stimulating an inflammatory response;
(iv) stimulating cytokine production;
(v) stimulating dendritic cell development, activity or mobilization; or
(vi) a combination of any of (i)-(v).

46.-47. (canceled)

48. The composition of claim 45, wherein
(i) the immune potentiator stimulates a Type I IFN response and is selected from the group consisting of STING, MAVS, IRF1, IRF3, IRF5, IRF7, IRF8, IRF9, TBK1, IKKα, IKKi, MyD88, TRAM, TRAF3, TRAF6, IRAK1, IRAK4, TRIF, IPS-1, RIG-1, DAI, IFI16, and a combination thereof;
(ii) the immune potentiator stimulates an NFκB-mediated proinflammatory response and is selected from the group consisting of STING, c-FLIP, IKKβ, RIPK1, Btk, TAK1, TAK-TAB1, TBK1, MyD88, IRAK1, IRAK2, IRAK4, TAB2, TAB3, TRAF6, TRAM, MKK3, MKK4, MKK6, MKK7, and a combination thereof;
(iii) the immune potentiator is an intracellular adaptor protein and is selected from the group consisting of STING, MAVS, MyD88, and a combination thereof;
(iv) the immune potentiator is an intracellular signaling protein of a TLR signaling pathway and is selected from the group consisting of MyD88, IRAK 1, IRAK2, IRAK4, TRAF3, TRAF6, TAK1, TAB2, TAB3, TAK-TAB1, MKK3, MKK4, MKK6, MKK7, IKKα, IKKβ, TRAM, TRIF, RIPK1, TBK1, and a combination thereof;
(v) the immune potentiator is IRF3, IRF7 or a combination thereof;
(vi) the immune potentiator is involved in necroptosis or necroptosome formation and is selected from the group consisting of MLKL, RIPK1, RIPK3, DIABLO, FADD, and a combination thereof;
(vii) the immune potentiator is involved in pyroptosis or inflammasome formation and is selected from the group consisting of caspase 1, caspase 4, caspase 5, caspase 11, GSDMD, NLRP3, Pyrin domain, ASC/PYCARD and a combination thereof;
(viii) the immune potentiator comprises a constitutively active human STING polypeptide;
(ix) the immune potentiator is a MAVS polypeptide; or
(x) the immune potentiator is a constitutively active IRF3 polypeptide.

49.-70. (canceled)

71. The composition of claim 45, wherein the antigen of interest is one or more tumor antigens and/or one or more personalized cancer antigens, wherein the personalized cancer antigen is a concatemeric cancer antigen comprised of 2-100 peptide epitopes.

72.-74. (canceled)

75. The composition of claim 71, wherein the concatemeric cancer antigen comprises one or more of:
a) the 2-100 peptide epitopes are interspersed by cleavage sensitive sites;
b) the mRNA encoding each peptide epitope is linked directly to one another without a linker;
c) the mRNA encoding each peptide epitope is linked to one or another with a single nucleotide linker;
d) each peptide epitope comprises 25-35 amino acids and includes a centrally located SNP mutation;
e) at least 30% of the peptide epitopes have a highest affinity for class I MHC molecules from a subject;
f) at least 30% of the peptide epitopes have a highest affinity for class II MHC molecules from a subject;
g) at least 50% of the peptide epitopes have a predicated binding affinity of IC >500 nM for HLA-A, HLA-B and/or DRB1;
h) the mRNA encodes 20 peptide epitopes;
i) 50% of the peptide epitopes have a binding affinity for class I MHC and 50% of the peptide epitopes have a binding affinity for class II MHC; and/or
j) the mRNA encoding the peptide epitopes is arranged such that the peptide epitopes are ordered to minimize pseudo-epitopes.

76.-79. (canceled)

80. The composition of claim 45, wherein (i) the enhanced immune response is a cellular immune response, humoral immune response or both;
(ii) the enhanced immune response stimulates cytokine production, stimulates antigen-specific CD8⁺ T cell responses, stimulates antigen-specific CD4⁺ helper cell responses, increases the effector memory CD62L^{lo} T cell population, stimulates B cell activity or stimulates antigen-specific antibody production, or any combination of the foregoing responses; or
(iii) the immune response to the antigen of interest is increased by a fold magnitude relative to the immune response to the antigen in the absence of the immune potentiator.

81.-97. (canceled)

98. A lipid nanoparticle comprising the composition of claim 45.

99.-106. (canceled)

107. A kit comprising a container comprising the lipid nanoparticle of claim 98, and an optional pharmaceutically acceptable carrier, and a package insert comprising instructions for administration of the lipid nanoparticle or composition for enhancing an immune response in an individual.

108.-116. (canceled)

117. A method for enhancing an immune response to an antigen of interest, the method comprising administering to a subject the lipid nanoparticle of claim 98 such that an immune response to the antigen of interest is enhanced in the subject.

118.-130. (canceled)
